SEQUENCE LISTING (163 sequences)

SEQ ID NO: 1 - protein, 305 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 2 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 3 - protein, 358 aa, Homo sapiens
CEEPPTFEAMELIGKPKPYYEIGERVDYKCKKGYFYIPPLATHTICDRNHTWLPVSDDACYRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKVLPPSSTKPPALSHSVSTSSTTKSPASSASGPRPTYKPPVSNYPGYPKPEEGILDSLDVWVIAVIVIAIVVGVAVICVVPYRYLQRRKKKGTYLTDETHREVKFTSL

SEQ ID NO: 4 - protein, 392 aa, Homo sapiens [sequence omitted]

SEQ ID NO: 5 - protein, 103 aa, Homo sapiens
LQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 6 - protein, 128 aa, Homo sapiens
MGIQGGSVLFGLLLVLAVFCHSGHSLQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 7 - protein, 77 aa, Homo sapiens
LQCYNCPNPTADCKTAVNCSSDFDACLITKAGLQVYNKCWKFEHCNFNDVTTRLRENELTYYCCKKDLCNFNEQLEN

SEQ ID NO: 8 - protein, 347 aa, Homo sapiens
DCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 9 - protein, 381 aa, Homo sapiens
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 10 - protein, 319 aa, Homo sapiens
DCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTS

SEQ ID NO: 11 - protein, 18 aa, Thosea asigna virus
EGRGSLLTCGDVEENPGP

SEQ ID NO: 12 - protein, 24 aa, Thosea asigna virus
LEGGGEGRGSLLTCGDVEENPGPR

SEQ ID NO: 13 - protein, 22 aa, Teschovirus A
GSGATNFSLLKQAGDVEENPGP

SEQ ID NO: 14 - protein, 19 aa, Teschovirus A
ATNFSLLKQAGDVEENPGP

SEQ ID NO: 15 - protein, 20 aa, Equine rhinitis A virus
QCTNYALLKLAGDVESNPGP

SEQ ID NO: 16 - protein, 22 aa, Foot-and-mouth disease virus
VKQTLNFDLLKLAGDVESNPGP

SEQ ID NO: 17 - protein, 21 aa, Thosea asigna virus
GSGEGRGSLLTCGDVEENPGP

SEQ ID NO: 18 - protein, 23 aa, Thosea asigna virus
AAGSGEGRGSLLTCGDVEENPGP

SEQ ID NO: 19 - RNA, 12 nt, synthetic construct
  source 1..12: mol_type = other RNA
gttttagagcta

SEQ ID NO: 20 - RNA, 63 nt, synthetic construct
  source 1..63: mol_type = other RNA
tagcaagttaaaataaggctagtccgttatcaacttgaaaaagtggcaccgagtcggtgcttt

SEQ ID NO: 21 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 23 - protein, 242 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 24 - protein, 12 aa, synthetic construct
ESKYGPPCPPCP

SEQ ID NO: 25 - protein, 25 aa, synthetic construct
TTTPAPRPPTPAPTIASQPLSLRPE

SEQ ID NO: 26 - protein, 39 aa, synthetic construct
IEVMYPPPYLDNEKSNGTIIHVKGKHLCPSPLFPGPSKP

SEQ ID NO: 27 - protein, 44 aa, synthetic construct
ACRPAAGGAVHTRGLDFACDIYIWAPLAGTCGVLLLSLVITLYC

SEQ ID NO: 28 - protein, 27 aa, synthetic construct
FWVLVVVGGVLACYSLLVTVAFIIFWV

SEQ ID NO: 29 - protein, 27 aa, synthetic construct
FWVLVVVGGVLACYSLLVTVAFIIFWV

SEQ ID NO: 30 - protein, 41 aa, synthetic construct
RSKRSRLLHSDYMNMTPRRPGPTRKHYQPYAPPRDFAAYRS

SEQ ID NO: 31 - protein, 42 aa, synthetic construct
KRGRKKLLYIFKQPFMRPVQTTQEEDGCSCRFPEEEEGGCEL

SEQ ID NO: 32 - protein, 242 aa, synthetic construct; identical to SEQ ID NO: 23

SEQ ID NO: 33 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 112 aa, synthetic construct; identical to SEQ ID NO: 21

SEQ ID NO: 35 - DNA, 20 bp, synthetic construct
  source 1..20: mol_type = other DNA
accccacagtggggccacta

SEQ ID NO: 36 - DNA, 20 bp, synthetic construct
  source 1..20: mol_type = other DNA
tgttggaaggatgaggaaat

SEQ ID NO: 37 - DNA, 20 bp, synthetic construct
  source 1..20: mol_type = other DNA
tcactatgctgccgcccagt

SEQ ID NO: 38 - RNA, 20 nt, synthetic construct
  source 1..20: mol_type = other RNA
tctctccatgtgcagtagga

SEQ ID NO: 39 - RNA, 20 nt, synthetic construct
  source 1..20: mol_type = other RNA
ctggatgtcggaggagtacg

SEQ ID NO: 40 - RNA, 20 nt, synthetic construct
  source 1..20: mol_type = other RNA
gtctccggaaactcgaggtg

SEQ ID NO: 41 - RNA, 20 nt, synthetic construct
  source 1..20: mol_type = other RNA
acagtgtagacttgattgac

SEQ ID NO: 42 - RNA, 20 nt, synthetic construct
  source 1..20: mol_type = other RNA
cgtgagtaaacctgaatctt

SEQ ID NO: 43 - RNA, 20 nt, synthetic construct
  source 1..20: mol_type = other RNA
gatattggcataagcctccc

SEQ ID NO: 44 - RNA, 20 nt, synthetic construct
  source 1..20: mol_type = other RNA
agagtctctcagctggtaca

SEQ ID NO: 45 - protein, 7 aa, synthetic construct
ATSNLAS

SEQ ID NO: 46 - protein, 9 aa, synthetic construct
QQWSFNPPT

SEQ ID NO: 47 - protein, 122 aa, synthetic construct
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 48 - protein, 5 aa, synthetic construct
SYNMH

SEQ ID NO: 49 - protein, 17 aa, synthetic construct
AIYPGNGDTSYNQKFKG

SEQ ID NO: 50 - protein, 246 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 51 - protein, 124 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 52 - protein, 10 aa, synthetic construct
GDSVSSNSAA

SEQ ID NO: 53 - protein, 9 aa, synthetic construct
TYYRSKWYN

SEQ ID NO: 54 - protein, 14 aa, synthetic construct
AREVTGDLEDAFDI

SEQ ID NO: 55 - protein, 107 aa, synthetic construct
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 56 - protein, 6 aa, synthetic construct
QTIWSY

SEQ ID NO: 57 - skipped

SEQ ID NO: 58 - protein, 9 aa, synthetic construct
QQSYSIPQT

SEQ ID NO: 59 - protein, 246 aa, synthetic construct
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 60 - protein, 124 aa, synthetic construct
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSS

SEQ ID NO: 61 - protein, 10 aa, synthetic construct
GDSVSSNSVA

SEQ ID NO: 62 - protein, 9 aa, synthetic construct
TYYRSTWYN

SEQ ID NO: 63 - protein, 14 aa, synthetic construct
AREVTGDLEDAFDI

SEQ ID NO: 64 - protein, 107 aa, synthetic construct
DIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 65 - protein, 6 aa, synthetic construct
QTIWSY

SEQ ID NO: 66 - skipped

SEQ ID NO: 67 - protein, 9 aa, synthetic construct
QQSYSIPQT

SEQ ID NO: 68 - protein, 246 aa, synthetic construct
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 69 - protein, 111 aa, synthetic construct
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 70 - protein, 15 aa, synthetic construct
RASESVSVIGAHLIH

SEQ ID NO: 71 - protein, 7 aa, synthetic construct
LASNLET

SEQ ID NO: 72 - protein, 9 aa, synthetic construct
LQSRIFPRT

SEQ ID NO: 73 - protein, 117 aa, synthetic construct
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 74 - protein, 5 aa, synthetic construct
DYSIN

SEQ ID NO: 75 - protein, 17 aa, synthetic construct
WINTETREPAYAYDFRG

SEQ ID NO: 76 - protein, 8 aa, synthetic construct
DYSYAMDY

SEQ ID NO: 77 - protein, 246 aa, synthetic construct
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 78 - protein, 111 aa, synthetic construct
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 79 - protein, 15 aa, synthetic construct
RASESVTILGSHLIY

SEQ ID NO: 80 - protein, 7 aa, synthetic construct
LASNVQT

SEQ ID NO: 81 - protein, 9 aa, synthetic construct
LQSRTIPRT

SEQ ID NO: 82 - protein, 117 aa, synthetic construct
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 83 - protein, 5 aa, synthetic construct
HYSMN

SEQ ID NO: 84 - protein, 17 aa, synthetic construct
RINTESGVPIYADDFKG

SEQ ID NO: 85 - protein, 8 aa, synthetic construct
DYLYSLDF

SEQ ID NO: 86 - protein, 121 aa, synthetic construct
EVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSS

SEQ ID NO: 87 - protein, 8 aa, synthetic construct
GFTFSSYA

SEQ ID NO: 88 - protein, 8 aa, synthetic construct
ISGSGDYI

SEQ ID NO: 89 - protein, 14 aa, synthetic construct
AKEGTGANSSLADY

SEQ ID NO: 90 - protein, 243 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 91 - protein, 106 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIK

SEQ ID NO: 92 - protein, 6 aa, synthetic construct
QSISSY

SEQ ID NO: 93 - skipped

SEQ ID NO: 94 - protein, 8 aa, synthetic construct
QQKYDLLT

SEQ ID NO: 95 - protein, 119 aa, synthetic construct
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 96 - protein, 10 aa, synthetic construct
GGSISSSSYY

SEQ ID NO: 97 - protein, 7 aa, synthetic construct
ISYSGST

SEQ ID NO: 98 - protein, 11 aa, synthetic construct
ARDRGDTILDV

SEQ ID NO: 99 - DNA, 1503 bp, synthetic construct
  source 1..1503: mol_type = other DNA
atggccttaccagtgaccgccttgctcctgccgctggccttgctgctccacgccgccaggccggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaaaatacgacctcctcacttttggcggagggaccaaggttgagatcaaaggcagcaccagcggctccggcaagcctggctctggcgagggcagcacaaagggacagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctcctatagtgggagcacctactacaacccgtccctcaagagtcgagtcaccatatccgtagacacgtccaagaaccagttctccctgaagctgagttctgtgaccgccgcagacacggcggtgtactactgcgccagagatcgtggagacaccatactagacgtatggggtcagggtacaatggtcaccgtcagctcattcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccaga

SEQ ID NO: 100 - protein, 501 aa, synthetic construct
MALPVTALLLPLALLLHAARPDIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 101 - protein, 21 aa, synthetic construct
  VARIANT 1..3: note = This region may be absent
GSGEGRGSLLTCGDVEENPGP

SEQ ID NO: 102 - protein, 22 aa, synthetic construct
  VARIANT 1..3: note = This region may be absent
GSGATNFSLLKQAGDVEENPGP

SEQ ID NO: 103 - protein, 23 aa, synthetic construct
  VARIANT 1..3: note = This region may be absent
GSGQCTNYALLKLAGDVESNPGP

SEQ ID NO: 104 - protein, 25 aa, synthetic construct
  VARIANT 1..3: note = This region may be absent
GSGVKQTLNFDLLKLAGDVESNPGP

SEQ ID NO: 105 - protein, 7 aa, synthetic construct
  VARIANT 5..7: note = This region may be absent
RRRRGSG

SEQ ID NO: 106 - protein, 7 aa, synthetic construct
  VARIANT 5..7: note = This region may be absent
RKRRGSG

SEQ ID NO: 107 - protein, 15 aa, synthetic construct
  VARIANT 5..7: note = This region may be absent
  VARIANT 13..15: note = This region may be absent
RKRRGSGTPDPWGSG

SEQ ID NO: 108 - skipped

SEQ ID NO: 109 - RNA, 12 nt, synthetic construct
  source 1..12: mol_type = other RNA
gttttagagcta

SEQ ID NO: 110 - skipped

SEQ ID NO: 111 - RNA, 66 nt, synthetic construct
  source 1..66: mol_type = other RNA
tagcaagttaaaataaggctagtccgttatcaacttgaaaaagtggcaccgagtcggtgctttttt

SEQ ID NO: 112 - skipped

SEQ ID NO: 113 - RNA, 17 nt, synthetic construct
  source 1..17: mol_type = other RNA
gtttsagagctatgctg

SEQ ID NO: 114 - skipped

SEQ ID NO: 115 - RNA, 71 nt, synthetic construct
  source 1..71: mol_type = other RNA
cagcatagcaagttsaaataaggctagtccgttatcaacttgaaaaagtggcaccgagtcggtgctttttt

SEQ ID NO: 116 - skipped

SEQ ID NO: 117 - RNA, 14 nt, synthetic construct
  source 1..14: mol_type = other RNA
gttttagtactctg

SEQ ID NO: 118 - skipped

SEQ ID NO: 119 - RNA, 64 nt, synthetic construct
  source 1..64: mol_type = other RNA
cagaatctactaaaacaaggcaaaatgccgtgtttatctcgtcaacttgttggcgagatttttt

SEQ ID NO: 120 - RNA, 88 nt, synthetic construct
  source 1..88: mol_type = other RNA
gtcgtctataggacggcgaggacaacgggaagtgccaatgtgctctttccaagagcaaacaccccgttggcttcaagatgaccgctcg

SEQ ID NO: 121 - skipped

SEQ ID NO: 122 - RNA, 23 nt, synthetic construct
  source 1..23: mol_type = other RNA
cgagcggtctgagaagtggcact

SEQ ID NO: 123 - skipped

SEQ ID NO: 124 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

SEQ ID NO: 125 - protein, 80 aa, synthetic construct
  VARIANT 1..80: note = This sequence may encompass 1-10 GGGSGGGS repeating units
GGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 126 - protein, 30 aa, synthetic construct
  VARIANT 1..30: note = This sequence may encompass 1-10 GSG repeating units
GSGGSGGSGGSGGSGGSGGSGGSGGSGGSG

SEQ ID NO: 127 - protein, 21 aa, synthetic construct
MALPVTALLLPLALLLHAARP

SEQ ID NO: 128 - protein, 20 aa, synthetic construct
METDTLLLWVLLLWVPGSTG

SEQ ID NO: 129 - protein, 22 aa, synthetic construct
MLLLVTSLLLCELPHPAFLLIP

SEQ ID NO: 130 - protein, 45 aa, synthetic construct
TTTPAPRPPTPAPTIASQPLSLRPEACRPAAGGAVHTRGLDFACD

SEQ ID NO: 131 - protein, 39 aa, synthetic construct; identical to SEQ ID NO: 26

SEQ ID NO: 132 - protein, 42 aa, synthetic construct
AAAIEVMYPPPYLDNEKSNGTIIHVKGKHLCPSPLFPGPSKP

SEQ ID NO: 133 - protein, 12 aa, synthetic construct
ESKYGPPCPPCP

SEQ ID NO: 134 - protein, 12 aa, synthetic construct
ESKYGPPCPSCP

SEQ ID NO: 135 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 136 - protein, 24 aa, synthetic construct
IYIWAPLAGTCGVLLLSLVITLYC

SEQ ID NO: 137 - protein, 27 aa, synthetic construct
FWVLVVVGGVLACYSLLVTVAFIIFWV

SEQ ID NO: 138 - protein, 28 aa, synthetic construct
MFWVLVVVGGVLACYSLLVTVAFIIFWV

SEQ ID NO: 139 - protein, 42 aa, synthetic construct; identical to SEQ ID NO: 31

SEQ ID NO: 140 - protein, 41 aa, synthetic construct; identical to SEQ ID NO: 30

SEQ ID NO: 141 - protein, 112 aa, synthetic construct; identical to SEQ ID NO: 33

SEQ ID NO: 142 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 143 - protein, 6 aa, synthetic construct
QDISKY

SEQ ID NO: 144 - protein, 9 aa, synthetic construct
QQGNTLPYT

SEQ ID NO: 145 - protein, 18 aa, synthetic construct
GSTSGSGKPGSGEGSTKG

SEQ ID NO: 146 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 147 - protein, 8 aa, synthetic construct
GVSLPDYG

SEQ ID NO: 148 - protein, 7 aa, synthetic construct
IWGSETT

SEQ ID NO: 149 - protein, 14 aa, synthetic construct
AKHYYYGGSYAMDY

SEQ ID NO: 150 - protein, 15 aa, synthetic construct
GGGGSGGGGSGGGGS

SEQ ID NO: 151 - DNA, 1467 bp, synthetic construct
  source 1..1467: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 152 - protein, 489 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 153 - DNA, 1458 bp, synthetic construct
  source 1..1458: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 154 - protein, 486 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 155 - DNA, 1383 bp, synthetic construct
  source 1..1383: mol_type = other DNA
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 156 - protein, 461 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 157 - DNA, 1467 bp, synthetic construct
  source 1..1467: mol_type = other DNA
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 158 - protein, 489 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 159 - protein, 246 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGSGKPGSGEGSTKGEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 160 - protein, 106 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 161 - protein, 10 aa, synthetic construct
RASSSVNYMD

SEQ ID NO: 162 - protein, 9 aa, unidentified
LAGLIDADG

SEQ ID NO: 163 - protein, 30 aa, synthetic construct
  VARIANT 1..30: note = This sequence may encompass 1-6 GGGGS repeating units
GGGGSGGGGSGGGGSGGGGSGGGGSGGGGS

(19) United States
(12) Patent Application Publication
(10) Pub. No.: US 2026/0159808 A1
(43) Pub. Date: Jun. 11, 2026

(54) HYPOIMMUNOGENIC MAIT CELLS, METHODS OF MAKING AND METHODS OF USING SAME

(71) Applicant: Sana Biotechnology, Inc., Seattle, WA (US)

(72) Inventors: Adam James JOHNSON, Seattle, WA (US); De-Kuan CHANG, Seattle, WA (US); Divy S. KANGEYAN, Seattle, WA (US); Pascal Remi Joseph BEAUCHESNE, Seattle, WA (US); Darin SALLOUM, Seattle, WA (US)

(21) Appl. No.: 19/126,591

(22) PCT Filed: Nov. 1, 2023
(86) PCT No.: PCT/US2023/036625

§ 371 Date: May 1, 2025

Related U.S. Application Data
(60) Provisional application No. 63/421,977, filed on Nov. 2, 2022.
(60) Provisional application No. 63/431,598, filed on Dec. 9, 2022.

(51) Int. Cl. C12N 5/0783; A61K 40/11; A61K 40/31; A61K 40/42; C12N 15/90

(52) U.S. Cl. CPC C12N 5/0636; A61K 40/11; A61K 40/31; A61K 40/4211; C12N 15/907; C12N 2510/00

(57) ABSTRACT

Methods of profiling cells for donor capability with a recipient subject are provided. In particular, methods relating to identifying cells, such as T cells, suitable for making a cell therapy product and administration to a patient (e.g., as CAR T cell therapy). Methods are also provided of profiling the donor capability of a cell or population of cells for cell therapy, such methods comprising evaluating the cell or the population of cells for predicted function. A predicted cell function may be evaluated by assaying one or more cell parameters. Further, cells and cell therapy products manufactured according to such methods are provided.

### CROSS REFERENCE TO RELATED APPLICATIONS

This application claims priority to U.S. Provisional Application No. 63/431,598, filed Dec. 9, 2022, and U.S. Provisional Application No. 63/421,977, filed Nov. 2, 2022, each of which is incorporated herein by reference.

### FIELD

The present disclosure relates to a method of manufacturing cell therapy products. In some embodiments, cells of a cell therapy product are obtained from a donor whose cells are suitable for use in a cell therapy product. In some embodiments, the present disclosure relates to identifying cells, such as T cells, suitable for making a cell therapy product and administration to a patient (e.g., as CAR T cell therapy). In some embodiments, the present disclosure relates to selecting cells, such as T cells, suitable for making a cell therapy product and administration to a patient (e.g., as CAR T cell therapy). Certain cells that are suitable for use in a cell therapy product are also provided.

### BACKGROUND

Healthy donor cells (e.g., T cells) contain a spectrum of biological activities, all of which contribute to final cell functionality. Identifying characteristics predictive for in vivo potency is essential in generating cells (e.g., CAR-T cells) for clinical application, whether as autologous or allogenic donor cells. However, the origins of improved donor cell capability are largely unknown. Currently, donors are chosen based on metrics associated with a donor patient, such as sufficient cell count for manufacturing, gender (e.g., female), blood group (e.g., O), age, body mass index and blood volume. However, this method for donor selection does not necessarily correlate with clinical outcome.

A need remains in the art for approaches that allow for more effective selection of donor cells for the manufacture of safe and efficacious cell therapy products.

Accordingly, an object of the present disclosure is to provide methods to more effectively predict clinical outcomes associated with donor cells. It is further an object of the present disclosure to provide methods for manufacturing cell therapy products having chacteristics associated with positive clinical outcomes for patients.

### SUMMARY

The present disclosure relates to the above-mentioned technical problem and provides embodiments as described herein, particularly by reference to the claims.

The present disclosure is based on the surprising and unexpected discovery that despite phenotypic similarities, cells have characteristics that lead to the selection and/or identification of cells with improved functionality (e.g., improved donor functionality). The present disclosure therefore provides methods comprising evaluating a predicted function of a cell or a population of cells, the predicted function being useful for profiling the functionality and/or suitability of a cell or population of cells (e.g., donor capability) for cell therapy. According to the surprising discovery of the disclosure, evaluating a predicted function of a cell or a population of cells can be based on assaying at least one cell parameter of the cell or the population of cells.

In addition to methods described herein, cells per se and populations of cells as described herein also are provided. Further, compositions, containers and kits as described herein are provided.

Inventive uses of assays for assaying at least one cell parameter of a cell or a population of cells also are provided.

In view of the advantages in applying the methods described herein to selecting a cell or population of cells for making a cell therapy, methods for making a cell therapy and methods of treating a disease or condition in a subject as described herein also are provided.

Related computer-implemented methods, systems, computer-readable mediums, data processing devices and computer programs also are provided as described herein.

### BRIEF DESCRIPTION OF THE DRAWINGS

Select embodiments of the disclosed technology will now be described in more detail with reference to the following figures:

FIG. 1 relates to an exemplary workflow for producing allogeneic hypoimmunogenic pluripotent (HIP) CAR-T cells. Allogeneic hypo-immune CAR T cells are generated from healthy donor T cells. As illustrated, T cells are engineered to overexpress CD47, a surface protein known as ‘don't eat me’ signal to macrophages and NK cells. In the embodiment shown, immune-identifiable proteins (TRAC, MHC class I and II) are removed to protect the patient from donor T cells and to ensure donor T cell protection from patient immune recognition.

FIG. 2 relates to certain T cell quality attributes assessed. Healthy donor T cells contain a spectrum of biological activities, many of which will contribute to the final T cell functionality. Identifying functional characteristics that are predictive for CAR T cell potency and safety will be essential in generating. HIP CAR T cells for clinical studies. The T cell assays that differentiate between donor performance and may be used to select high-quality T cells for clinical trials.

FIG. 3 relates to an exemplary Incucyte assay and readout for T cell quality assessment. HIP CAR-T cells generated from different donors show differential efficacy upon serial tumor challenge assay. (A) Assay set up: Serial tumor challenge assays may be performed using an Incucyte platform. As shown, in some embodiments, CAR-T cells undergo several rounds of rechallenge, and both T and target cell count is evaluated. The assay evaluates several aspects of T cell functionality including serial tumor killing ability, long term expansion capacity, and T cell exhaustion. (B) Representative assay readout: HIP CAR-Ts generated from different donors demonstrated differential efficacy upon serial tumor challenge assay. Donor differences were identified at the low Effector to Target ratio (1:8). Top panel- target cell growth, Bottom panel- T cell growth. (C) and (D) relate to 4 data points relevant to T cell functionality which can be summarized in index fashion. (C) Average growth rate (proliferation index) of target cell growth (top panel) or T cell growth (bottom panel), calculated as the geometric mean of fold change for each stimulation cycle. Interpretation of the proliferation index is the average growth rate (tumor or T cell) across ‘n’ stimulations. Success being measured as a low tumor cell proliferation index and a higher T cell proliferation index through the assay (assuming a reverse correlation between tumor cell and T cell growth in assay). (D) T cell durability of response (top panel)/growth (bottom panel) is a slope of the fold change of restimulation cycles for each target (top panel)/T cell (bottom panel). Slope values that reach above 0 indicate cell growth, values maintained at 0 indicate steady-state growth, and slopes below 0 indicate a reduction in cell proliferation across multiple tumor challenge cycles. (E) Summary of Incucyte assay data based on 6 donors.

FIG. 4 relates to a MesoScale Discovery assay with cytokine production as an exemplary readout for T cell quality. HIP CAR-Ts generated from different donors produce defined quantities of cytokines. (A) Assay set up. Cytokine measurement readout may be performed using a MesoScale Discovery assay. Production of 4 groups (e.g., effector, stimulatory, regulatory, inflammatory) of cytokines was evaluated. (B) Representative assay readout: Cytokine production assay demonstrated differences in functionality of HIP CAR-T cells generated from different donors. Large arrow indicates that donor 3 CAR-T cells produced the least amount of various cytokines. Data is represented as pg cytokine produced per mL of collected supernatant. (C) Representative assay readout: HIP CAR-Ts generated from different donors produce defined quantities across 4 groups of cytokines. Data is represented as pg cytokine produced per T cell in assay. (D) Summary based on characterization of 5 donors.

FIG. 5 relates to exemplary in vivo efficacy testing. HIP CAR-Ts generated from different donors show efficacy in vivo at high and low CAR-T doses. (A) Assay set up. In vivo evaluation of HIP CAR T cells was performed using NSG-Nalm-6 mouse model. (B) Representative assay readout: HIP CAR-Ts generated from 7 different donors showed efficacy based on tumor cell growth control (represented as area under the curve (AUC)) at high (top panel—5e6 cells/mouse) and low (bottom panel—0.5e6 cells/mouse) CAR-T dose. (C) Summary based on AUC values of FIG. 5B high CAR-T dose for 7 donors.

FIG. 6 relates to exemplary gene expression profiling of Allo-T donors/nCounter data. HIP CAR-Ts generated from different donors show distinct gene expression profiles that correlated with functional performance. (A) Assay set up. Gene expression was evaluated using nCounter CAR-T panel. 4 timepoints for both CD4 and CD8 T cells were evaluated: APH (unmodified, isolated CD4 and CD8 T cells); CAR-resting (Thawed, HIP edited CD4 and CD8 CAR T cells prior to use in IncuCyte assay). CAR-activated (HIP edited CD4 and CD8 CAR T cells isolated two days after incorporation into the IncuCyte assay); CAR-exhausted (HIP edited CD4 and CD8 CAR T cells isolated at the end of the four rechallenges of the IncuCyte assay). (B) Gene expression profiling comparing poor and excellent donors identified by the IncuCyte, MesoScale Discovery, and in vivo assays identified signature readouts that correlate with better functional performance.

FIG. 7 relates to exemplary T cell quality attributes that correlate with functional in vitro and in vivo efficacy.

FIG. 8 relates to exemplary single-cell cytokine production via IsoPlexis analysis. HIP CAR-Ts generated from different donors produced variable levels and concentrations of cytokine. (A) Two criteria were evaluated in the IsoPlexis assay; the polyfunctionality strength index (PSI), a measure of signal intensity of cytokine production by T cell populations that produce 2+ cytokines; the multifunctional index, a measure of cells that produce greater than 4 cytokines/cell. (B) Multifunctional heat map that identifies differences in HIP edited CD4 and CD8 CAR T cells to produce various combinations of cytokines.

FIG. 9 relates to an exemplary HIP CAR T persistence study in humanized mice. Compared to CAR T cells (without HIP modifications) and unmodified T cells, HIP CAR T cells show longer survival, greater cell persistence, and function even after re-injection (after 83 days) in humanized mice. (A) Study design. At day 0, Nalm6 tumor cells (expressing luciferase for imaging) were introduced into humanized mice. At day 3, one of the below groups of cells were introduced. At day 83, Nalm6 tumor cells (expressing luciferase for imaging) were reintroduced. (B) Table showing the 3 different types of cells that were analysed (HIP CAR T cells (CD19 CAR), CAR T cells (CAR-EGFRt transgene) with no HIP edits, and T cells (unmodified)), each in 5 mice. The HIP CAR T cells were those derived from donor 6. (C) Bioluminescent imaging data showing the presence of tumor cells (expressing luciferase) in injected mice at day 0, 15, 27, 55, 75, 83 and 87 (unless the mice had already been sacrificed due to tumor growth). (D) Bioluminescent data showing the light signal emitted (photons/sec) of the tumor cells. (E) Flow cell analysis data of CAR+ cells in bone marrow in CAR T-injected humanized mice and HIP CAR T-injected humanized mice, at the time point that the animal was sacrificed. (F) Flow cell analysis data of CAR+ cells in spleen in CAR T-injected humanized mice and HIP CAR T-injected humanized mice, at the time point that the animal was sacrificed. (G) Flow cell analysis data of CD19+ tumor cells in bone marrow in tumor only (Nalm only) injected humanized mice, unmodified T cell (Mock-T) injected humanized mice, CAR T injected humanized mice and HIP CAR T-injected humanized mice, at the time point that the animal was sacrificed. (H) Summary flow cell analysis data of CAR+ cells in bone marrow and in spleen in CAR T-injected humanized mice and HIP CAR T-injected humanized mice from the timepoints. HIP CAR T (from primary T) persistence study in humanized mice—3 months follow up with Nalm6 re-injection. HIP+ CAR T cells are more likely to survive in the bone marrow and spleen of humanized mice than HIP− CAR T cells (at day of sacrifice). (I) Summary flow cell analysis data of CD19+ tumor cells in bone marrow in tumor only (Nalm only)injected humanized mice, unmodified T cell (Mock-T) injected humanized mice, CAR T injected humanized mice and HIP CAR T-injected humanized mice from the timepoints. HIP CAR T (from primary T) persistence study in humanized mice—3 months follow up with Nalm6 re-injection. Mice treated with HIP+ CAR T cells show significantly reduced nalm6 tumor in bone marrow (at day of sacrifice).

FIG. 10A is a flow chart of an exemplary method of evaluating a cell or a population of cells for a predicted function.

FIG. 10B is a block diagram of an exemplary computing device that may be used to perform methods described herein.

FIG. 11 is a block diagram of an example network environment for use in the methods and systems described herein.

FIG. 12 is a block diagram of an example computing device and an example mobile computing device for use with methods and systems described herein.

FIG. 13 relates to the survival of exemplary bulk HIP CAR T cells in humanized mice. While injected bulk HIP showed <50% HIP cells, on D95 all surviving CAR+ T cells have the HIP phenotype.

FIG. 14 relates to analysis of the survival and function of exemplary HIP cells in compositions containing contamination with other cells. Specifically, FIG. 14 shows that human HIP islet cells in mixed p-islets with wt (unedited) or DKO (paritally edited) cells still survive in immunocompetent, allogeneic, diabetic humanized mice. a-f, Mixed human wt and HIP p-islets were transplanted into allogeneic, diabetic humanized mice. When the wt cells were Luc₊, BLI pictures (a) and signals (b) showed a vanishing of this cell population over time (all 5 animals are shown). When the HIP cells were Luc₊, BLI pictures (d) and signals (e) showed cell survival (all 5 animals are shown). In both cases, the transplanted mixed p-islets achieved glycemic control (c, f, 5 animals per group). g-l, Mixed human DKO and HIP p-islets were transplanted into allogeneic, diabetic humanized mice. When the DKO cells were Luc₊, BLI pictures (g) and signals (h) showed a vanishing of this cell population over time (all 6 animals are shown). When the HIP cells were Luc₊, BLI pictures (j) and signals (k) showed cell survival (all 5 animals are shown). In both cases, the transplanted mixed p-islets achieved glycemic control (1, 6 animals, 1, 5 animals).

FIG. 15 relates to an exemplary analysis of in vivo functionality, in vitro functionality, and donor cell characteristics (as described in Examples 1 to 6) in relation to CAR T anti-tumor activity. Specifically, FIG. 15a shows the relative strengths and limitations of exemplary cell parameters and readouts usefulness in predicting donor cell quality. Meanwhile, FIG. 15b shows the framework and data inputs for an exemplary multidimensional T cell quality analysis.

FIG. 16 relates to exemplary schematic of potential assays that can be performed to evaluate CAR T cell pre-clinical functionality.

FIGS. 17a and 17b relate to exemplary heat maps of various levels, signals, and scores to characterize donor samples having attributes correlating with pre-clinical performance of HIP CAR T cells.

FIG. 18 relates to an exemplary ranking of weighted performance values of thirteen (13) T cell donors.

FIGS. 19a and 19b relate to exemplary analyses of the proportion of CD4+ central memory (TCM) T cells and CD8+ TCM cells, respectively, in whole blood draw samples collected from donors.

FIGS. 20a and 20b relate to exemplary analyses of T cell proliferation (as measured by geometric mean) and the proportion of active caspase 3-positive CD4+ T cells, respectively, following leukapheresis of donor blood samples.

FIGS. 21a and 21b relate to exemplary analyses of the proportion of NK cells (CD56+) in the CD8+ cell population and effector memory cells (TEM) in the CD4+ cell population, respectively, following enrichment of donor blood draw samples for CD4+ and CD8+ T cells.

FIG. 22 relates to exemplary analyses of the proportion of CD3+ cells (left panel), CD4+ cells (center panel), and CD8+ cells (right panel) of the CD45+ cell population of peripheral blood mononuclear cells (PBMCs) isolated by apheresis of donor blood draw samples.

FIG. 23 relates to an exemplary expression profile analysis of donor gene profiles using a Nanostring nCounter panel of 794 genes relating to T cell and CAR T biology.

FIG. 24 relates to an exemplary clinical manufacturing process for a hypoimmune, CD-19 directed, allogeneic chimeric antigen receptor (CAR) T cell product. Specifically, FIG. 24a depicts a flow chart of an exemplary general manufacturing process. FIG. 24b is a summary of various cell attributes assayed after 3 complete manufacturing runs.

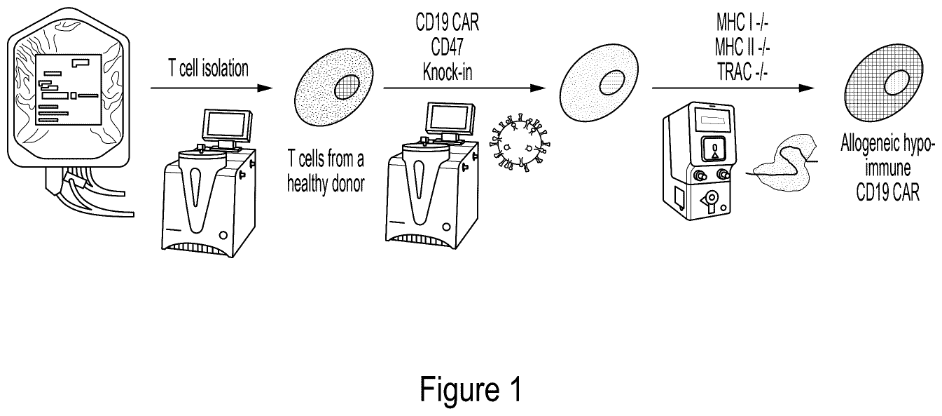

### DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The term “about” as used herein when referring to a measurable value, such as an amount or concentration and the like, is meant to encompass variations of 20%, 10%, 5%, 1%, 0.5%, or even 0.1% of the specified amount. As used herein, including in the appended claims, the singular forms “a.” “or.” and “the” include plural referents unless the context clearly dictates otherwise. For example, “a” or “an” means “at least one” or “one or more.” It is understood that aspects and variations described herein include embodiments “consisting of” and/or “consisting essentially of” such aspects and variations.

As used herein, the term “and/or” includes any and all combinations of one or more of the associated listed items.

As used herein, the term “exogenous” with reference to a polypeptide or a polynucleotide is intended to mean that the referenced molecule is introduced into the cell of interest. The exogenous molecule, such as exogenous polynucleotide, can be introduced, for example, by introduction of an exogenous encoding nucleic acid into the genetic material of the cells such as by integration into a chromosome or as non-chromosomal genetic material such as a plasmid or expression vector. Therefore, the term as it is used in reference to expression of an encoding nucleic acid refers to introduction of the encoding nucleic acid in an expressible form into the cell. In some cases, an “exogenous” molecule is a molecule, construct, factor and the like that is not normally present in a cell but can be introduced into a cell by one or more genetic, biochemical or other methods.

The term “endogenous” refers to a referenced molecule, such as a polynucleotide (e.g., gene), or polypeptide, that is present in a native or unmodified cell. For instance, the term when used in reference to expression of an endogenous gene refers to expression of a gene encoded by an endogenous nucleic acid contained within the cell and not exogenously introduced.

A “gene,” includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. The sequence of a gene is typically present at a fixed chromosomal position or locus on a chromosome in the cell.

“Genomic instability” refers to the presence of an increased frequency, number, or significance of nucleotide (e.g., DNA) alterations. In some embodiments, genomic instability is associated with alterations ranging from a single nucleotide to whole chromosome changes. Exemplary chromosome changes include nucleotide instability (NIN), microsatellite instability (MIN or MSI), and chromosomal instability (CIN). NIN is characterized by an increased frequency of base substitutions, deletions, and insertions of one or a few nucleotides. MIN or MSI is the result of defects in mismatch repair genes which leads to the expansion and contraction of short nucleotide repeats called microsatellites. CIN leads to changes in both chromosome number and structure.

Methods for detecting, assessing and/or measuring genomic instability are generally known by those of skill in the art. For example, those of skill in the art will appreciate that detection of genome instability can be achieved using a variety of technologies, ranging from single-cell approaches to high-throughput multicellular techniques, each capable of detecting different levels of genomic changes. Those of skill in the art will also appreciate that any method capable of detecting chromosomal, microsatellite, or nucleotide changes may be adequate to measure a component of genomic instability. Such methods include, but are not limited to, karyotyping, flow cytometry, single nucleotide polymorphism (SNP) arrays, genome sequencing (including for example NGS based sequencing), and polymerase chain reaction (PCR). See Pikor, L., et al., “The detection and implication of genome instability in cancer,” Cancer Metastasis Rev (2013) 32:341-352, which is incorporated herein by reference in its entirety.

The term “locus” refers to a fixed position on a chromosome where a particular gene or genetic marker is located. Reference to a “target locus” refers to a particular locus of a desired gene in which it is desired to target a genetic modification, such as a gene edit or integration of an exogenous polynucleotide.

The term “expression” with reference to a gene or “gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or can be a protein produced by translation of an mRNA. Gene products also include RNAs which are modified, by processes such as capping, polyadenylation, methylation, and editing, and proteins modified by, for example, methylation, acetylation, phosphorylation, ubiquitination. ADP-ribosylation, myristoylation, and glycosylation. Hence, reference to expression or gene expression includes protein (or polypeptide) expression or expression of a transcribable product of or a gene such as mRNA. The protein expression may include intracellular expression or surface expression of a protein. Typically, expression of a gene product, such as mRNA or protein, is at a level that is detectable in the cell.

As used herein, a “detectable” expression level, means a level that is detectable by standard techniques known to a skilled artisan, and include for example, differential display, RT (reverse transcriptase)-coupled polymerase chain reaction (PCR), Northern Blot, and/or RNase protection analyses as well as immunoaffinity-based methods for protein detection, such as flow cytometry, ELISA, or western blot. The degree of expression levels need only be large enough to be visualized or measured via standard characterization techniques.

As used herein, the term “increased expression”, “enhanced expression” or “overexpression” means any form of expression that is additional to the expression in an original or source cell that does not contain the modification for modulating a particular gene expression, for instance a wild-type expression level (which can be absence of expression or immeasurable expression as well). Reference herein to “increased expression,” “enhanced expression” or “overexpression” is taken to mean an increase in gene expression and/or, as far as referring to polypeptides, increased polypeptide levels and/or increased polypeptide activity, relative to the level in a cell that does not contain the modification, such as the original source cell prior to the engineering to introduce the modification, such as an unmodified cell or a wild-type cell. The increase in expression, polypeptide levels or polypeptide activity can be at least 5%, 10%, 20%, 30%, 40% or 50%, 60%, 70%, 80%, 85%, 90%, or 100% or even more. In some cases, the increase in expression, polypeptide levels or polypeptide activity can be at least 2-fold, 5-fold, 10-fold, 20-fold, 30-fold, 40-fold, 50-fold, 60-fold, 70-fold, 80-fold, 90-fold, 100-fold, 200-fold or more.

The term “hypoimmunogenic” refers to a cell that is less prone to immune rejection by a subject to which such cells are transplanted. For example, relative to a similar cell that does not contain modifications, such as an unaltered or unmodified wild-type cell, such a hypoimmunogenic cell may be about 2.5%,5%, 10%, 20%, 30%, 40%, 50%, 60%, 70%, 80%, 90%, 95%, 97.5%, 99% or more less prone to immune rejection by a subject into which such cells are transplanted. Typically, the hypoimmunogenic cells are allogenic to the subject and a hypoimmunogenic cell evades immune rejection in an MHC-mismatched allogeneic recipient. In some embodiments, a hypoimmunogenic cell is protected from T cell-mediated adaptive immune rejection and/or innate immune cell rejection.

Hypoimmunogenicity of a cell can be determined by evaluating the immunogenicity of the cell such as the cell's ability to elicit adaptive and innate immune responses. Such immune response can be measured using assays recognized by those skilled in the art.

The term “tolerogenic factor” as used herein include immunosuppressive factors or immune-regulatory factors that modulate or affect the ability of a cell to be recognized by the immune system of a host or recipient subject upon administration, transplantation, or engraftment. Typically, a tolerogenic factor is a factor that induces immunological tolerance to an engineered primary cell so that the engineered primary cell is not targeted, such as rejected, by the host immune system of a recipient. Hence, a tolerogenic factor may be a hypoimmunity factor. Examples of tolerogenic factors include immune cell inhibitory receptors (e.g., CD47), proteins that engage immune cell inhibitory receptors, checkpoint inhibitors and other molecules that reduce innate or adaptive immune recognition

The terms “decrease,” “reduced,” “reduction,” and “decrease” are all used herein generally to mean a decrease by a statistically significant amount. However, for avoidance of doubt, “decrease.” “reduced,” “reduction,” “decrease” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased,” “increase” or “enhance” or “activate” are all used herein to generally mean an increase by a statically significant amount; for the avoidance of any doubt, the terms “increased.” “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “modification” refers to any change or alteration in a cell that impacts gene expression in the cell. In some embodiments, the modification is a genetic modification that directly changes the gene or regulatory elements thereof encoding a protein product in a cell, such as by gene editing, mutagenesis or by genetic engineering of an exogenous polynucleotide or transgene.

As used herein, “indel” refers to a mutation resulting from an insertion, deletion, or a combination thereof, of nucleotide bases in the genome. Thus, an indel typically inserts or deletes nucleotides from a sequence. As will be appreciated by those skilled in the art, an indel in a coding region of a genomic sequence will result in a frameshift mutation, unless the length of the indel is a multiple of three. A CRISPR/Cas system of the present disclosure can be used to induce an indel of any length in a target polynucleotide sequence.

In some embodiments, the alteration is a point mutation. As used herein, “point mutation” refers to a substitution that replaces one of the nucleotides. A CRISPR/Cas system of the present disclosure can be used to induce an indel of any length or a point mutation in a target polynucleotide sequence.

As used herein, “knock out” includes deleting all or a portion of the target polynucleotide sequence in a way that interferes with the function of the target polynucleotide sequence. For example, a knock out can be achieved by altering a target polynucleotide sequence by inducing an indel in the target polynucleotide sequence in a functional domain of the target polynucleotide sequence (e.g., a DNA binding domain). Those skilled in the art will readily appreciate how to use the CRISPR/Cas systems of the present disclosure to knock out a target polynucleotide sequence or a portion thereof based upon the details described herein.

In some embodiments, the alteration results in a knock out of the target polynucleotide sequence or a portion thereof. Knocking out a target polynucleotide sequence or a portion thereof using a CRISPR/Cas system of the present disclosure can be useful for a variety of applications. For example, knocking out a target polynucleotide sequence in a cell can be performed in vitro for research purposes. For ex vivo purposes, knocking out a target polynucleotide sequence in a cell can be useful for treating or preventing a disorder associated with expression of the target polynucleotide sequence (e.g., by knocking out a mutant allele in a cell ex vivo and introducing those cells comprising the knocked out mutant allele into a subject).

By “knock in” herein is meant a process that adds a genetic function to a host cell. This causes increased levels of the knocked in gene product, e.g., an RNA or encoded protein. As will be appreciated by those in the art, this can be accomplished in several ways, including adding one or more additional copies of the gene to the host cell or altering a regulatory component of the endogenous gene increasing expression of the protein is made. This may be accomplished by modifying the promoter, adding a different promoter, adding an enhancer, or modifying other gene expression sequences.

In some embodiments, an alteration or modification described herein results in reduced expression of a target or selected polynucleotide sequence. In some embodiments, an alteration or modification described herein results in reduced expression of a target or selected polypeptide sequence.

In some embodiments, an alteration or modification described herein results in increased expression of a target or selected polynucleotide sequence. In some embodiments, an alteration or modification described herein results in increased expression of a target or selected polypeptide sequence.

“Modulation” of gene expression refers to a change in the expression level of a gene. Modulation of expression can include, but is not limited to, gene activation and gene repression. Modulation may also be complete, i.e., wherein gene expression is totally inactivated or is activated to wildtype levels or beyond; or it may be partial, wherein gene expression is partially reduced, or partially activated to some fraction of wildtype levels.

The term “operatively linked” or “operably linked” are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence but need not be directly adjacent to it. For example, an enhancer is a transcriptional regulatory sequence that is operatively linked to a coding sequence, even though they are not contiguous.

The terms “polypeptide” and “protein,” as used herein, may be used interchangeably to refer to a series of amino acid residues joined by peptide bonds (i.e., a polymer of amino acid residues), and are not limited to a minimum length. Such polymers may contain natural or non-natural amino acid residues, or combinations thereof, and include, but are not limited to, peptides, polypeptides, oligopeptides, dimers, trimers, and multimers of amino acid residues. Thus, a protein or polypeptide includes include those with modified amino acids (e.g., phosphorylated, glycated, glycosylated, etc.) and amino acid analogs. Full-length polypeptides or proteins, and fragments thereof, are encompassed by this definition. The terms also include modified species thereof, e.g., post-translational modifications of one or more residues, for example, methylation, phosphorylation glycosylation, sialylation, or acetylation.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For instance, where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure. In some embodiments, two opposing and open-ended ranges are provided for a feature, and in such description it is envisioned that combinations of those two ranges are provided herein. For example, in some embodiments, it is described that a feature is greater than about 10 units, and it is described (such as in another sentence) that the feature is less than about 20 units, and thus, the range of about 10 units to about 20 units is described herein.

As used herein, a “subject” or an “individual,” which are terms that are used interchangeably, is a mammal. In some embodiments, a “mammal” includes humans, non-human primates, domestic and farm animals, and zoo, sports, or pet animals, such as dogs, horses, rabbits, cattle, pigs, hamsters, gerbils, mice, ferrets, rats, cats, monkeys, etc. In some embodiments, the subject or individual is human. In some embodiments, the subject is a patient that is known or suspected of having a disease, disorder or condition.

As used herein, the term “treating” and “treatment” includes administering to a subject an effective amount of cells described herein so that the subject has a reduction in at least one symptom of the disease or an improvement in the disease, for example, beneficial or desired clinical results. For purposes of this technology, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition but may not be a complete cure for the disease. In some embodiments, one or more symptoms of a disease or disorder are alleviated by at least 5%, at least 10%, at least 20%, at least 30%, at least 40%, or at least 50% upon treatment of the disease.

For purposes of this technology, beneficial or desired clinical results of disease treatment include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable.

A “vector” or “construct” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors. Methods for the introduction of vectors or constructs into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

Within the context of the present disclosure, the term “average” (or similar) refers to either a predetermined value or is calculated on the basis of a set of, e.g., samples, data points, results, scores, or indices. For example, in some embodiments, an “average” can be a mean, median, or mode value.

Within the context of the present disclosure, the term “profiling” (or similar) refers to assessing the characteristics of a population of cells. For example, characteristics relating to cell quality. Profiling may include further categorisation.

Within the context of the present disclosure, the term “donor capability” (or similar) refers to the potential of one or more cells to have therapeutic effectiveness. Donor capability is assessed on the basis of cell parameters in accordance with the methods described herein.

Within the context of the present disclosure, the term “donor” (or similar) refers to a cell or a population of cells which are “self” or “non-self”. In this way, donor cells can be used for autologous or allogenic cell therapy.

Within the context of the present disclosure, the term “edited” (or similar) refers to modified or engineered cells i.e., cells that have been altered in some way.

Within the context of the present disclosure, the term “exceptional” is used interchangeably with the term “excellent”.

In the present disclosure, the term “and/or” when used in a list may be taken also to disclose the combination of any one item from the list with any one or more of the other items in the list. In other words, “A, B and/or C” may be taken also to disclose ‘A+B’, ‘A+C’, and ‘B+C’ (in addition to ‘A or B or C’ and ‘A+B+C’).

References to subject-matter disclosed or described “herein” relates to subject-matter disclosed or described anywhere in the present application.

### DETAILED DESCRIPTION

The present disclosure has identified variability between populations of donor cells and has further identified that profiling (e.g., multiparametric profiling) donor cells is an effective predictor for donor capability and therefore for identifying cells suitable for administration to a patient as a cell therapy.

According to the methods described herein, it will be understood that profiling donor cells therefore provides a hitherto unrecognised ability to identify a high-quality population of cells for making a cell therapy product based on the profiled donor capability of said cell population.

Within the wide-ranging applications of the concepts and methods described herein, application to the field of allogeneic cell therapy is of importance. Furthermore, application of the concepts and methods described herein to the field of CAR-T cells (e.g., allogeneic CAR T-cell therapy) is of importance. Furthermore, application of the concepts and methods described herein to the field of hypoimmune (HIP) T cells is of importance.

According to the methods described herein, cells are provided that are useful for allogenic donor selection, such as for HIP CAR-T manufacturing. These ‘universal’ donor cells can persist in patients and deliver therapeutic efficacy without the need for immunosuppression to limit graft rejection.

A number of methods are provided herein.

In one aspect, the present disclosure provides a method comprising assaying at least one cell parameter of the cell or the population of cells and evaluating a predicted function of the cell or the population of cells based on the at least one cell parameter.

In one aspect, the present disclosure provides a method of profiling the donor capability of a cell or a population of cells for cell therapy, the method comprising assaying at least one cell parameter of the cell or the population of cells and evaluating a predicted function of the cell or the population of cells based on the at least one cell parameter.

In one aspect, the present disclosure provides a method of identifying a cell or a population of cells for making a cell therapy product, the method comprising assaying at least one cell parameter of the cell or the population of cells and evaluating a predicted function of the cell or the population of cells based on the at least one cell parameter.

In one aspect, the present disclosure provides a method of identifying a cell or a population of cells suitable for administration to a subject as a cell therapy, the method comprising assaying at least one cell parameter of the cell or the population of cells and evaluating a predicted function of the cell or the population of cells based on the at least one cell parameter.

In one aspect, the present disclosure provides a method of selecting a cell or a population of cells for making a cell therapy product, the method comprising assaying at least one cell parameter of the cell or the population of cells and evaluating a predicted function of the cell or the population of cells based on the at least one cell parameter.

In one aspect, the present disclosure provides a method of selecting a cell or a population of cells suitable for administration to a subject as a cell therapy, the method comprising assaying at least one cell parameter of the cell or the population of cells and evaluating a predicted function of the cell or the population of cells based on the at least one cell parameter.

In one aspect the one or more of the cell parameters comprises:
a. cell activation;
b. cell polyfunctionality or cell multifunctionality;
c. cell cytotoxicity;
d. cell growth rate;
e. durability of cell growth;
f. durability of cell response;
g. the cell's ability to elicit adaptive and innate immune responses;
h. characteristics associated with a particular cell type (e.g., cell marker characterization, biomarker, intracellular markers, extracellular markers, cell cytokine production, antibody production);
i. cell cytokine production;
j. cell safety attributes;
k. cell viability;
l. cell impurity level(s);
m. immune cell identity;
n. immune cell subtyping;
o. cell subtype ratio;
p. cell proliferation;
q. HLA typing; and/or
r. transcriptome.

In some embodiments, the the safety attributes comprise mycoplasma contamination; sterility; endotoxin level; karyotype; RCL (replication competent lentivirus) detection; VCN (vector copy number); and/or virus screening.

In one aspect, the present disclosure provides a method of profiling the donor capability of a cell or population of cells for cell therapy, the method comprising evaluating the cell or the population of cells for predicted function.

In one aspect, the present disclosure provides a method of identifying a cell or a population of cells for making a cell therapy product, the method comprising evaluating the cell or the population of cells for predicted function.

In one aspect, the present disclosure provides a method of identifying a cell or a population of cells suitable for administration to a subject as a cell therapy, the method comprising evaluating the cell or the population of cells for predicted function.

In one aspect, the present disclosure provides a method of selecting a cell or a population of cells for making a cell therapy product, the method comprising evaluating the cell or the population of cells for predicted function.

In one aspect, the present disclosure provides a method of selecting a cell or a population of cells suitable for administration to a subject as a cell therapy, the method comprising evaluating the cell or the population of cells for predicted function.

In some embodiments, evaluating the cell or the population of cells predicted cell function comprises evaluating the cell or the population of cells for predicted in vivo cell functioning.

In some embodiments, the predicted cell function comprises at least one of the cell functions selected from the group consisting of: cell persistence, engraftment, durability of cell response, potency, cell safety attributes, cell viability, cell impurity level(s), and immunogenicity. In some embodiments, the predicted cell function comprises cell persistence. In some embodiments, the predicted cell function comprises engraftment. In some embodiments, the predicted cell function comprises durability of cell response. In some embodiments, the predicted cell function comprises potency. In some embodiments, the predicted cell function comprises cell safety attributes. In some embodiments, the predicted cell function comprises cell viability. In some embodiments, the predicted cell function comprises cell impurity level(s). In some embodiments, the predicted cell function comprises immunogenicity.

In a method described herein, predicted cell function may comprise at least one of the cell functions selected from the group consisting of: a level of cell persistence, a level of engraftment, a level of durability of cell response, a level of potency, a level of cell safety attributes, a level of cell viability, a level of cell impurity level(s) and a level of immunogenicity.

Predicted cell function may be evaluated in the methods described herein by assaying at least one cell parameter selected from the group consisting of: cell activation; cell polyfunctionality; cell multifunctionality; cell cytotoxicity; cell growth rate; durability of cell growth; durability of cell response; the cell's ability to elicit adaptive and innate immune responses; characteristics associated with the particular cell type (e.g., cell marker characterization, biomarker, intracellular markers, extracellular markers, cell cytokine production, antibody production); cell cytokine production; cell safety attributes; cell viability; cell impurity level(s), immune cell identity, immune cell subtyping, cell subtype ratio, cell proliferation, HLA typing, and transcriptome.

In some embodiments, predicted cell function is evaluated according to the methods described herein by assaying one or more cell parameters. Optionally, the assayed cell parameters comprise: cell activation; cell polyfunctionality or multifunctionality; cell cytotoxicity; cell growth rate; durability of cell growth; durability of cell response; the cell's ability to elicit adaptive and innate immune responses; characteristics associated with particular cell type (e.g., cell marker characterization, biomarker, intracellular markers, extracellular markers, cell cytokine production, antibody production); cell cytokine production; cell safety attributes; cell viability; and/or cell impurity level(s), immune cell identity, immune cell subtyping, cell subtype ratio, cell proliferation, HLA typing, and a transcriptome.

In some embodiments, predicted cell function is evaluated by assaying cell activation. In some embodiments, predicted cell function is evaluated by assaying cell polyfunctionality. In some embodiments, predicted cell function is evaluated by assaying cell multifunctionality. In some embodiments, predicted cell function is evaluated by assaying cell cytotoxicity. In some embodiments, predicted cell function is evaluated by assaying cell growth rate. In some embodiments, predicted cell function is evaluated by assaying durability of cell growth. In some embodiments, predicted cell function is evaluated by assaying durability of cell response. In some embodiments, predicted cell function is evaluated by assaying the cell's ability to elicit adaptive and innate immune responses. In some embodiments, predicted cell function is evaluated by assaying characteristics associated with particular cell type (e.g., cell marker characterization, biomarker, intracellular markers, extracellular markers, cell cytokine production, antibody production). In some embodiments, predicted cell function is evaluated by assaying cell cytokine production. In some embodiments, predicted cell function is evaluated by assaying antibody production. In some embodiments, predicted cell function is evaluated by assaying cell safety attribute. In some embodiments, predicted cell function is evaluated by assaying cell viability. In some embodiments, predicted cell function is evaluated by assaying cell impurity level(s). In some embodiments, predicted cell function is evaluated by assaying immune cell identity. In some embodiments, predicted cell function is evaluated by assaying immune cell subtyping. In some embodiments, predicted cell function is evaluated by assaying cell subtype ratio. In some embodiments, predicted cell function is evaluated by assaying cell proliferation. In some embodiments, predicted cell function is evaluated by assaying HLA typing. In some embodiments, predicted cell function is evaluated by assaying transcriptomic profile.

Predicted cell function may be evaluated in the methods described herein by assaying multiple cell parameters including cell activation.

Predicted cell function may be evaluated in the methods described herein by assaying multiple cell parameters including cell polyfunctionality.

Predicted cell function may be evaluated in the methods described herein by assaying multiple cell parameters including cell multifunctionality.

Predicted cell function may be evaluated in the methods described herein by assaying multiple cell parameters including cell cytotoxicity.

Predicted cell function may be evaluated in the methods described herein by assaying multiple cell parameters including cell growth rate.

Predicted cell function may be evaluated in the methods described herein by assaying multiple cell parameters including durability of cell growth.

Predicted cell function may be evaluated in the methods described herein by assaying multiple cell parameters including durability of cell response, i.e., the ability of the cell or the population of cells to continue to respond to challenge (e.g., serial challenge).

Predicted cell function may be evaluated in the methods described herein by assaying multiple cell parameters including the cell's ability to elicit adaptive and innate immune responses.

Predicted cell function may be evaluated in the methods described herein by assaying multiple cell parameters including characteristics associated with the particular cell type (e.g., cell marker characterization, biomarker, intracellular markers, extracellular markers, cell cytokine production, antibody production).

Predicted cell function may be evaluated in the methods described herein by assaying multiple cell parameters including cell cytokine production. Predicted cell function may be evaluated in the methods described herein by assaying multiple cell parameters including antibody production.

Predicted cell function may be evaluated in the methods described herein by assaying multiple cell parameters including cell safety attributes.

Predicted cell function may be evaluated in the methods described herein by assaying multiple cell parameters including cell viability.

Predicted cell function may be evaluated in the methods described herein by assaying multiple cell parameters including cell impurity level(s).

Predicted cell function may be evaluated in the methods described herein by assaying multiple cell parameters including immune cell identity.

Predicted cell function may be evaluated in the methods described herein by assaying multiple cell parameters including immune cell subtyping.

Predicted cell function may be evaluated in the methods described herein by assaying multiple cell parameters including cell subtype ratio.

Predicted cell function may be evaluated in the methods described herein by assaying multiple cell parameters including cell proliferation.

Predicted cell function may be evaluated in the methods described herein by assaying multiple cell parameters including HLA typing.

Predicted cell function may be evaluated in the methods described herein by assaying multiple cell parameters including transcriptomic profile.

In some embodiments, the safety attributes comprise mycoplasma contamination; sterility; endotoxin level; karyotype; RCL (replication competent lentivirus) detection; VCN (vector copy number); and/or virus screening.

In some embodiments, the method comprises assaying at least 2 cell parameters. In some embodiments, the method comprises assaying 2, 3, 4, 5.6, 7, 8, 9 or 10 cell parameters.

In some embodiments, assaying comprises an in vitro assay, an in vivo assay, an immune assay, a cell activity assay, a cell avidity assay, a cell proliferation assay, a cell cytotoxicity assay, a cellular stress assay, a tumor challenge assay, an expression assay, a cytokine production assay, transcriptomic profiling assay, a proteomic profiling assay, a genomic profiling assay, a genomic stability assay, an epigenetic profiling assay, a cell developmental potential profiling assay, a cell subtyping assay; a cell receptor profiling assay; a cell antibody production assay; a cell antibody profiling assay; a cell viability assay; a cell killing assay; a cytokine dependent growth assay; and/or a cytokine independent growth assay.

In some embodiments, the method comprises two or more of the above assays. In some embodiments, the method comprises 2, 3, 4, 5, 6, 7, 8, 9, 10, 11, 12, 13, 14, 15, 16, 17,18, 19, 20, 21, 22, 23.24 or 25 of the assays.

In some embodiments, the immune assay comprises a T cell proliferation assay, a T cell activation assay, a T cell killing assay, an NK cell proliferation assay, an NK cell activation assay, and/or a macrophage activity assay.

In some embodiments, assaying according to the methods described herein comprises calculating one or more assay readouts.

Optionally, the one or more assay readouts comprise a cell functionality score, a cell polyfunctionality index, a cell multifunctionality index, an in vivo efficacy score, an in vivo activity score, an in vivo response score, an in vitro efficacy score, an in vitro activity score, an in vitro response score, an immune efficacy score, an immune activity score, an immune response score, a cell activity score, a cell activity response score, a cell specificity score, a cell sensitivity score, a cell avidity score, a cell proliferation score, a cell proliferative index, a cell cytotoxicity score, a cell cytotoxicity response score, a cell stress score, a cell stress response score, a tumor challenge efficacy score, a tumor challenge activity score, a tumor challenge response score, a tumor challenge specificity score, a tumor challenge sensitivity score, an expression profile, an expression signature, an expression signal, an expression score, a bulk cytokine or chemokine production profile, a bulk cytokine or chemokine production signature, a bulk cytokine or chemokine production profile, a bulk cytokine or chemokine production signal, a bulk cytokine or chemokine production score, a single cell cytokine or chemokine production profile, a single cell cytokine or chemokine production signature, a single cell cytokine or chemokine production profile, a single cell cytokine or chemokine production signal, a single cell cytokine or chemokine production score, a transcriptomic profile, a transcriptomic signature, a transcriptomic signature, a transcriptomic score, a pathway profile, a pathway signature, a pathway signal, a pathway score, a proteomic profile, a proteomic signature, a proteomic signal, a proteomic score, a genomic profile, a genomic signature, a genomic signal, a genomic score, a genomic stability profile, a genomic stability signature, a genomic stability signal, a genomic stability score, an epigenetic profile, an epigenetic signature, an epigenetic signal, an epigenetic score, a cell developmental potential assessment, a cell developmental potential profile, a cell developmental potential signature, a cell developmental potential score, a cell subtyping profile, a cell subtyping signature, a cell subtyping score, a cell receptor profile, a cell receptor signature, a cell receptor signal; a cell receptor score; a cell antibody production score; a cell antibody profiling profile; a cell viability score; a cell viability profile; a cell killing score; a cytokine dependent growth score; a cytokine independent growth score; immune cell identity profile, immune cell subtyping profile, cell subtype ratio profile, cell proliferation profile, cell proliferation score. HLA typing profile, and transcriptomic profile.

In some embodiments, any or all or any combination of the scores/indexes are calculated.

In the methods described herein, the data regarding each of the one or more cell characteristics that has been assayed can be used to provide corresponding inputs to be processed (e.g., by a computer implemented algorithm) to evaluate predicted cell function for making a cell therapy.

For certain assays, the data from the assay may be a numerical value which can be used directly as an ‘assay readout’ input value for a (e.g., computer-implemented) method to evaluate predicted cell function.

For other assays, the data from the assay may not be a numerical value or a single value. Therefore, to the extent that a method as described herein requires an assay readout to be provided as a single value or a numerical value in order to be useful for evaluating predicted cell function, for example, in some embodiments relating to certain computer-implemented methods to evaluate predicted cell function, data from the assay may be converted to a single value or a numerical value. For example, data such as pixilation from a data image or flow plot data, etc. may be converted into a single value or a numerical value so that the single value or the numerical value can provide an input value for certain computer-implemented methods to evaluate predicted cell function.

The term ‘assay readout’ as used herein encompasses data output generated directly from an assay (e.g., one or more qualitative or quantitative values), as well as a single value or a numerical value obtained after conversion of data output generated directly from an assay.

In the methods described herein, an assay readout for a particular cell or population of cells may, in some instances, be compared to that of a reference cell or reference population of cells i.e., a reference value. The concept of reference cells and reference populations of cells, as well as reference values obtained therefrom, will be readily understood by a person skilled in the art and are discussed in further detail elsewhere herein.

In some embodiments, the method further comprises determining the cell type of the cell or the cell types in the population of cells. In some embodiments, the cell type or cell types are a cell subtype or cell subtypes.

In some embodiments, determining the cell type or the cell types comprise characterizing one or more expressed molecules. In some embodiments, the one or more expressed molecules are cell surface molecules.

In some embodiments, one or more of the assay readouts are one or more values suitable for use in a processor or a computer-implemented method to evaluate predicted cell function. In some embodiments, one or more of the assay readouts is a single value. In some embodiments, one or more of the assay readouts are one or more values which require conversion to numerical values. In some embodiments, one or more of the assay readouts are numerical values. In some embodiments, the assaying further comprises using assay readouts and a processor or a computer-implemented method to evaluate predicted cell function. In some embodiments, the evaluation of predicted cell function is provided as a single value. In some embodiments, evaluating predicted cell function comprises categorising the cell or the population of cells. In some embodiments, the evaluation of predicted cell function is provided as a single value and the cell or the population of cells is categorised based on the value.

In some embodiments, the evaluating predicted cell function comprises categorising the cell or the population of cells using a scale for donor capability. In some embodiments, the cells or the population of cells are categorised based on the number of cells in the population that meet the values in the scale. In some embodiments, the scale comprises 2, 3, 4, 5, 6, 7, 8, 9, 10 or more than 10 points.

The concept of a scale for profiling or evaluating data will be well understood by a person skilled in the art. In the methods disclosed herein, a scale may be used as a means to evaluate a cell or population of cells for predicted function and thus to identify a cell or a population of cells for making a cell therapy product. In this sense, a threshold value may be used in a scale whereby the scale threshold value distinguishes cells/cell populations above the threshold value that are useable for making a cell therapy product versus cells/cell populations below the cut-off point that are not useable. A threshold value may also be used in a scale whereby the scale threshold value distinguishes cells/cell populations above the threshold value that might be selected for making a cell therapy product versus cells/cell populations below the cut-off point that are not selected.

A scale may be used to rank and therefore compare the predicted cell function of a number of cells/cell populations. A scale may be employed such that cells/cell populations that are to be ranked are distributed evenly across the scale. Alternatively, a scale may be employed such that a position (point) on the scale corresponds to a particular score or score range of predicted cell function and any cell/cell population that is measured to have that predicted cell function score or a score within that range will be placed at that position on the scale.

For a 2-point scale, this may be used, for example, to denote cells/cell populations predicted to have the highest function versus the cells/cell populations predicted to have the lowest function. Alternatively, a 2-point scale may be used to denote cells/cell populations selected for making a cell therapy product versus cells/cell populations not selected. Similarly, a 2-point scale may be used to denote cells/cell populations useable for making a cell therapy product versus cells/cell populations not useable. Such 2-point scales may have a sub-scale further categorising the cells/cell populations within the 2-point scale.

For a 3-point scale, this may be used, for example, to denote cells/cell populations predicted to have the highest function versus the cells/cell populations predicted to have mid-level function and cells/cell populations predicted to have the lowest function. Alternatively, a scale with at least 3 points may be used to denote cells/cell populations having exceptional versus good versus poor predicted cell function, whereby cells/cell populations ranked as poor on the scale are not selected for making a cell therapy product. Exceptional predicted cell function may also be referred to herein as excellent predicted cell function. Cells/cell populations ranked as good on the scale may be selected for further assaying or further profiling in order to determine whether to select the cells/cell populations for making a cell therapy product. Such an ‘exceptional/good/poor’ 3-point scale may have a sub-scale further categorising the cells/cell populations within the 3-point scale.

It will be readily understood that a scale may be devised as appropriate with up to 10 points or even more than 10 points.

In the methods described herein, any further assaying or further profiling of the cells/cell populations may result in re-classifying the cells/cell populations in the appliable scale (e.g., based solely on the additional assay readouts from the further assays, based on assay readouts from the further and the original testing, etc.). The further profiling of the cells/cell populations may be based on characteristics of the donor e.g., sex or age of the donor. Females may be prioritised as donors, in some embodiments, post adolescent females, and in some embodiments, pre-menopausal females.

Further assaying or further profiling of a cell/cell population identified as ‘useable,’ ‘good,’ or ‘exceptional’ may be used as a means to determine whether to proceed with making a cell therapy product using that cell/cell population.

In the methods described herein, the value for predicted cell function for an assayed cell or population of cells may, in some instances, be compared to a that of a reference cell or reference population of cells i.e., a reference value. The skilled person will readily be able to select a technically appropriate reference cell or reference population of cells. However, it will be understood that the reference cell or reference population of cells should be of the same cell type, or composed of the same cell types, as the assayed cell or population of cells. Generally, the reference cell or reference population of cells should be of the same cell sub-type, or composed of the same cell subtypes, as the assayed cell or population of cells. Where the assayed cell or population of cells is genetically modified, comparison to either genetically modified reference cell or reference population of cells or unmodified reference cell or reference population of cells is permissible.

The reference value may be an average value calculated from a number of reference cells or reference populations of cells.

In some embodiments, the evaluating predicted cell function comprises categorising the cell or the population of cells as having poor, good or exceptional donor capability. In some embodiments, the cells or the population of cells are ranked and categorised as (i) poor if they fall below a reference value, (ii) good if they meet the reference value and (iii) exceptional if they exceed the reference value. In some embodiments, the cells or the population of cells are ranked and categorised as (i) non-useable if they fall below a reference value, and (ii) usable if they meet or exceed the reference value.

A variety of parameters may be used to categorise a cell or population of cells is as usable (or even exceptional) for making a cell therapy product.

In some embodiments, the cell or the population of cells is categorised as exceptional or usable if the cell or the population of cells shows a high amount of mucosal-associated invariant T (MAIT) relative to a cell or population of cells.

In some embodiments, the cell or the population of cells is categorised as exceptional or usable if the cell or the population of cells shows a high amount of T Cell Receptor Beta Variable 28 (TRBV28) relative to a cell or population of cells.

In some embodiments, the cell or the population of cells is categorised as exceptional or usable if the cell or population of cells shows a high amount of Interleukin-17A (IL17A) relative to a cell or population of cells.

In some embodiments, the cell or the population of cells is categorised as exceptional or usable if the cell or the population of cells is less activated and has a reduced NK-like signature relative to a reference cell or population of cells.

In some embodiments, the cell or the population of cells is categorised as exceptional or usable if the cell or population of cells has an activated phenotype and/or Th1/Tc1 and TH17/Tc17 states.

It will be understood that profiling the donor capability and evaluating the cell or the population of cells for predicted function may be carried out at any suitable stage of the overall process for making a cell therapy product. Specifically, evaluating a predicted function of the cell or the population of cells based on assaying at least one cell parameter may be carried out at any suitable stage of the overall process for making a cell therapy product.

In some embodiments, the evaluating predicted cell function is performed before cell modification, after cell modification, or both before and after cell modification. In some embodiments, the evaluating predicted cell function is performed before cell modification. In some embodiments, the evaluating predicted cell function is performed after cell modification. In some embodiments, the evaluating predicted cell function is performed both before and after cell modification. In some embodiments, further cell modification may be performed following the evaluating predicted cell function performed after cell modification; further optionally wherein evaluating predicted cell function also is performed after the further cell modification.

In some embodiments, the evaluating predicted cell function is performed on the cell or the population of cells prior to any genomic modifications.

In some embodiments the cell or the population of cells are cryopreserved prior to any genomic modifications and evaluating predicted cell function is performed before the cell or the population of cells is cryopreserved, and/or after the cell or the population of cells has been cryopreserved and thawed.

In some embodiments the cell or the population of cells are stored prior to any genomic modifications and evaluating predicted cell function is performed before the cell or the population of cells is stored, and/or after the cell or the population of cells has been stored.

In some embodiments the evaluating predicted cell function is performed on the cell or the population of cells prior to any hypoimmunogenic modifications.

In some embodiments, the cell or the population of cells are cryopreserved prior to any hypoimmunogenic modifications and the evaluating predicted cell function is performed: before the cell or the population of cells is cryopreserved, and/or after the cell or the population of cells has been cryopreserved and thawed.

In some embodiments, the cell or the population of cells are stored prior to any hypoimmunogenic modifications and evaluating predicted cell function is performed: before the cell or the population of cells is stored, and/or after the cell or the population of cells has been stored.

In some embodiments, the evaluating predicted cell function is performed after the cell or the population of cells have been modified.

In some embodiments, the cell or the population of cells are cryopreserved after any modification and the evaluating predicted cell function is performed: before the cell or the population of cells is cryopreserved, and/or after the cell or the population of cells has been cryopreserved and thawed.

In some embodiments, the cell or the population of cells are stored after any modification and evaluating predicted cell function is performed: before the cell or the population of cells is stored, and/or after the cell or the population of cells has been stored.

In some embodiments, the evaluating predicted cell function is performed after the cell or the population of cells have completed modifications.

In some embodiments, the cell or the population of cells are cryopreserved after completed modification and the evaluating predicted cell function is performed: before the cell or the population of cells is cryopreserved, and/or after the cell or the population of cells has been cryopreserved and thawed.

In some embodiments, the cell or the population of cells are stored after completed modification and evaluating predicted cell function is performed: before the cell or the population of cells is stored, and/or after the cell or the population of cells has been stored.

In some embodiments, the evaluating predicted cell function is performed before cell differentiation, after cell differentiation, or both before and after cell differentiation.

In some embodiments, the evaluating predicted cell function is performed on the cell or the population of cells prior to any differentiation.

In some embodiments, the cell or the population of cells is cryopreserved prior to any differentiation and the evaluating predicted cell function is performed: before the cell or the population of cells is cryopreserved, and/or after the cell or the population of cells has been cryopreserved and thawed.

In some embodiments, cell or the population of cells are stored prior to any differentiation and evaluating predicted cell function is performed: before the cell or the population of cells is stored, and/or after the cell or the population of cells has been stored.

In some embodiments, the evaluating predicted cell function is performed after the cell or the population of cells have been differentiated.

In some embodiments, the cell or the population of cells is cryopreserved after differentiation and the evaluating predicted cell function is performed: before the cell or the population of cells is cryopreserved, and/or after the cell or the population of cells has been cryopreserved and thawed.

In some embodiments, the cell or the population of cells are stored after differentiation and evaluating predicted cell function is performed: before the cell or the population of cells is stored, and/or after the cell or the population of cells has been stored.

In some embodiments, the evaluating predicted cell function is performed after the cell or the population of cells have completed differentiation.

In some embodiments, the cell or the population of cells are cryopreserved after completed differentiation and the evaluating predicted cell function is performed: before the cell or the population of cells is cryopreserved, and/or after the cell or the population of cells has been cryopreserved and thawed.

In some embodiments, the cell or the population of cells are stored after completed differentiation and evaluating predicted cell function is performed: before the cell or the population of cells is stored, and/or after the cell or the population of cells has been stored.

In this regard, it will be understood that evaluating predicted cell function may be carried out at any suitable point in a method of making a cell therapy product, from immediately after cell collection through to immediately prior to cell therapy administration. Furthermore, evaluating predicted cell function may be carried out only once or at multiple points in a method of making a cell therapy product.

Evaluating predicted cell function pre-editing is of particular interest in the present methods. Evaluating predicted cell function after partial cell editing also is of particular interest in the present methods. Evaluating predicted cell function following completion of cell editing also is of particular interest in the present methods.

In some embodiments, evaluating predicted cell function is performed pre-editing as well as after partial cell editing. In some embodiments, evaluating predicted cell function is performed pre-editing as well as following completion of cell editing. In some embodiments, evaluating predicted cell function is performed after partial cell editing as well as following completion of cell editing. In some embodiments, evaluating predicted cell function is performed pre-editing; after partial cell editing; and following completion of cell editing.

In some embodiments, the evaluating predicted cell function is performed before cell activation, after cell activation, or both before and after cell activation. In some embodiments, the evaluating predicted cell function is performed before cell activation. In some embodiments, the evaluating predicted cell function is performed after cell activation. In some embodiments, the evaluating predicted cell function is performed both before and after cell activation.

It will be known in the art that methods for manufacturing a cell therapy product may incorporate a step of measuring the cell or population of cells against ‘release criteria’ which enables the cell or population of cells to be approved for administration to a subject. Such ‘release criteria’ tests (sometimes referred to as a ‘release panel’) may relate to. e.g., cell safety-related attributes (e.g., mycoplasma contamination; sterility; endotoxin level; karyotype; RCL (replication competent lentivirus) detection; VCN (vector copy number); and/or virus screening) and/or cell impurity levels(s). Such ‘release criteria’ tests may readily be combined with assaying at least one cell parameter of the cell or the population of cells and evaluating a predicted function of the cell or the population of cells as described in the methods for profiling a population of cells for donor capability disclosed herein. In some instances, a categorising, ranking, single value cut-off, profiling scale, or single value (e.g., single number values) for assessing donor capability as described in the methods for profiling a population of cells for donor capability according to the present disclosure may take into account ‘release criteria’ tests such as safety-related attributes and/or cell impurity levels(s).

In one example, profiling according to a method as described herein may be carried out pre-cell modification (where formulating the cell therapy product then occurs post-cell modification). In one example, profiling as described herein may be carried out pre-cell modification and further profiling according to a method as described herein may be carried out post-cell modification (where formulating the cell therapy product then occurs post-cell modification). In one example, profiling as described herein may be carried out pre-cell modification and further profiling based on modification(s) may be carried out post-cell modification (where formulating the cell therapy product then occurs post-cell modification).

In one example, profiling according to a method as described herein may be carried out post-cell modification (where formulating the cell therapy product then occurs post-cell modification). In one example, profiling based on modification(s) as well as profiling according to a method as described herein may be carried out post-cell modification (where formulating the cell therapy product then occurs post-cell modification).

Suitable methods for selecting a cell or the population of cells based on based on modification(s), include, for example, selecting based on determining the presence of one or more modifications in the cell or the level of the one or more modifications in the population of cells.

In some embodiments, the method further comprises identifying the cell or the population of cells as suitable for administration to a subject as a cell therapy if the cell or the population of cells is determined as being ‘good’ or ‘exceptional’, or ‘usable’.

In some embodiments, the method further comprises identifying the cell or the population of cells as suitable for making a cell therapy product if the cell or the population of cells is determined as being ‘good’ or ‘exceptional’ or ‘usable’.

In some embodiments, further profiling is carried out if the cell or the population of cells is determined as being ‘good’ or ‘useable’

In some embodiments, the further profiling comprises assaying as described herein at least one of the cell parameters not previously assayed.

In some embodiments, the further profiling comprises assaying as described herein at least one of the same cell parameters as described herein previously assayed using an assay that was not previously used.

In some embodiments, the further profiling comprises assaying as described herein at least one of the same cell parameters previously assayed using an assay that was previously used.

In some embodiments, the further profiling comprises evaluating predicted cell function as described herein using at least one of the assays as described herein. In some embodiments, the further profiling comprises evaluating predicted cell function as described herein using at least 2, 3, 4, 5, 6, 7, 8, 9, 10 or more of the assays.

In some embodiments, the cell or the population of cells is identified as suitable for administration to a subject or suitable for making a cell therapy product if the further profiling determines the cell or the population of cells as having an exceptional, a good, or usable score.

In some embodiments, the cell or the population of cells is identified as suitable for administration to a subject or suitable for making a cell therapy product if addition of the score from the further profiling changes the overall categorisation to exceptional.

In some embodiments, the cell or the population of cells is identified as suitable for administration to a subject or suitable for making a cell therapy product if the cell or the population of cells is determined as having a further desirable characteristic.

In some embodiments, the cell or the population of cells is identified as suitable for administration to a subject or suitable for making a cell therapy product the individual from which the donor cells originated has a further desirable characteristic.

In some embodiments, the desirable characteristic is that the individual is female.

In some embodiments, the individual is female. In some embodiments the female is pre-menopausal.

In some embodiments the individual is between 18 and 35 years old.

In some embodiments, individual is between 18 and 40 years old, 18 and 30 years old, or 18 and 25 years old.

In some embodiments, the individual is age 18, 24, 28, 35, or 36.

In some embodiments, the individual has a BMI in the range 15-50, 15-45, 15-40, 15-35, 15-30, 15-25, or 15-20.

In some embodiments, the individual has a BMI of 22, 24, 34, 39, 41, or 42.

In some embodiments the desirable characteristic is based on the health history of the donor.

In some embodiments, the method further comprises identifying cells predicted to have in vivo functionality as described herein.

In some embodiments, the method further comprises selecting cells predicted to have in vivo functionality as described herein.

Further detail on assays that may be used in the methods described herein is provided below.

In some embodiments, the assaying comprises:
a) measuring (i) growth rate and/or (ii) durability of cell growth and/or (iii) durability of cell response in the cell or the population of cells, optionally using a real-time quantitative live-cell analysis platform, optionally measuring (i) growth rate and (ii) durability of cell growth and (iii) durability of cell response, optionally using a real-time quantitative live-cell analysis platform, optionally using one or more restimulation cycles;
b) measuring bulk cytokine production for a panel of cytokines in the cell or the population of cells, optionally using a multiplex cytokine detection technique, an electrochemiluminescence (ECL) detection technique and/or ELISA;
c) conducting single cell cytokine profiling on the cell or the population of cells, optionally using a multiplex proteomics assay;
d) conducting gene expression profiling on the cell or the population of cells to determine activation level (i) in a pre-modification resting state of the cell or the population of cells and/or (ii) in a post-modification resting state of the cell or the population of cells and/or (iii) in a post-modification activated state of the cell or the population of cells and/or (iv) in a post tumor challenge exhausted state, optionally in a post-modification resting state of the cell or the population of cells;
optionally using direct digital detection of mRNA molecules of interest; and/or optionally conducting gene expression profiling to determine activation level (i) in a pre-modification resting state of the cell or the population of cells and (ii) in a post-modification resting state of the cell or the population of cells and (iii) in a post-modification activated state of the cell or the population of cells and (iv)in a post tumor challenge exhausted state; and/or
e) measuring the response of the cell or the population of cells in vivo, optionally transplanting the cell or the population of cells into an allogeneic host and monitoring for cell activity;
optionally transplanting the cell or the population of cells into an allogeneic host and monitoring for cell escape from the host immune system;
optionally transplanting the cell or the population of cells into an allogeneic host and monitoring for cell growth;
optionally transplanting the cell or the population of cells into an allogeneic host and monitoring for cell rejection;
optionally transplanting the cell or the population of cells into an allogeneic host and monitoring for cell survival;
optionally transplanting the cell or the population of cells and a target cell or a population of target cells into an allogeneic host and monitoring for cell activity; optionally transplanting the cell or the population of cells and a target cell or a population of target cells into an allogeneic host and monitoring for cell escape from the host immune system;
optionally transplanting the cell or the population of cells and a target cell or a population of target cells into an allogeneic host and monitoring for cell growth;
optionally transplanting the cell or the population of cells and a target cell or a population of target cells into an allogeneic host and monitoring for cell rejection;
optionally transplanting the cell or the population of cells and a target cell or a population of target cells into an allogeneic host and monitoring for cell survival; and/or
optionally transplanting the cell or the population of cells into an allogeneic host and monitoring for teratoma formation; and/or
f) measuring the response of a host to the cell or the population of cells, optionally transplanting the cell or the population of cells into an allogeneic host, obtaining immune cells from the allogeneic host, and determining an activation state of the immune cells from the allogeneic host; and/or
optionally transplanting the cell or the population of cells into an allogeneic host, obtaining a blood sample from the allogeneic host, and determining a humoral response of the allogeneic host;
g) cell safety attributes; and/or cell impurity level(s).

In the methods described herein, any suitable real-time quantitative live-cell analysis platform may be employed, for example an Incucyte® assay.

In the methods described herein, any suitable means may be employed for measuring bulk cytokine production for a panel of cytokines. For example, a multiplex cytokine detection technique, an electrochemiluminescence (ECL) detection technique and/or ELISA may be employed. An exemplary electrochemiluminescence (ECL) detection technique is Meso Scale Discovery (MSD).

In the methods described herein, cytokine production may be measured, for example, using an E:T ratio of 1:1, 1:2, 1:3, 1:4, 1:5, 1:6, 1:7, 1:8, 1:9, 1:10, 1:12, 1:15, 1:16, or 1:20.

In the methods described herein, any suitable single cell cytokine profiling technique may be employed, for example the Isoplexis® Human Adaptive Immune assay.

In the methods described herein, any suitable gene expression profiling technique may be employed. Gene expression profiling may be carried out using direct digital detection of mRNA molecules of interest, for example using the nCounter® Sprint assay using target-specific, color-coded probe pairs.

In embodiments, the gene expression profile comprises at least 3 genes. In embodiments, the gene expression profile comprises at least 4 genes. In embodiments, the gene expression profile comprises at least 5 genes. In embodiments, the gene expression profile comprises at least 6 genes. In embodiments, the gene expression profile comprises at least 7 genes. In embodiments, the gene expression profile comprises at least 8 genes. In embodiments, the gene expression profile comprises at least 9 genes. In embodiments, the gene expression profile comprises at least 10 genes. In embodiments, the gene expression profile comprises more than 10 genes.

A reference gene expression profile as referred to herein typically is a pre-determined gene expression signature based on known desired features i.e., associated with acceptable or desired functional performance. The skilled person will be able to readily identify a desired gene expression profile for the particular cell type. Similarity of the gene expression profile for the assayed cell/cell population to the reference gene expression profile may be determined. This may then be used as a means to compare the assayed cell/cell population to that of one or more other assayed cells/cell populations. Similarity to a reference gene expression profile may be determined across all genes in the panel. In embodiments, similarity to a reference gene expression profile may be found when at least 2 genes of a 3 gene panel, at least 3 genes of a 5 gene panel, at least 4 genes of a 7 gene panel, at least 5 genes of a 9 gene panel, or at least 6 genes of a 10 gene panel are observed.

Similarity to a reference gene expression profile may be determined by any suitable means. In this regard, reference might be made to U.S. Pat. No. 7,873,481 B2 (the contents of which are incorporated herein by reference), As disclosed in U.S. Pat. No. 7,873,481 B2, evaluation includes all techniques which allow drawing conclusions based on the validated results with respect to the presence or absence of at least one specific compound or its chemical nature (qualitative analysis) (e.g., as a marker/biomarker) or the precise or relative amount of the at least one compound (quantitative analysis). Moreover, the conclusion can encompass a conclusion as to the degree of identity of the compounds or amounts thereof in different samples. In an aspect, evaluation, thus, also encompasses comparing validated results of different samples. In embodiments, the comparing comprises assessing whether the samples are different or identical to each other (i.e. the degree of similarity is determined). In principle, any statistical test which allows determining whether compounds or characteristic features thereof or amounts thereof will vary significantly between different samples is suitable for carrying out the aforementioned comparison. In some embodiments, suitable techniques include a pattern recognition algorithm and/or a statistical test algorithm and/or a multivariate algorithm eg. Principal Component Analysis (PCA), Simple Component Analysis (SCA), Independent Component Analysis (ICA), Principal Component Regression (PCR), Partial Least Squares (PLS), PLS Discriminant Analysis (PLS-DA), Support Vector Machines (SVM), Neural Networks, Bayesian Networks, Bayesian Learning Networks, Mutual Information, Backpropagation Networks, symmetrical Feed-Forward Networks, Self-Organizing Maps (SOMs), Genetic Algorithms, Hierarchical or K-Mean Clustering, Anova, Student's t-Test, Kruskal-Wallis Test, Mann-Whitney Test, Tukey-Kramer Test or Hsu's Best Test. The comparison of samples as described above can be applied to determine differences or similarities between samples with respect to their qualitative or quantitative composition. Determining of similarities may also encompass determining of mean or median values for the abundance of compounds. Comparison as used herein may, in the latter case, also comprise comparing the means or medians of two pluralities of samples suspected to differ in their compositions. Evaluation as used in accordance with the present disclosure can be assisted by automation, e.g., by a suitable computer program for at least one of the aforementioned algorithms on a computer. The following algorithms for evaluation can be, in whole or partially, carried out by a computer program containing instructions which allow for evaluation when implemented and carried out by a computer and/or a computer network or a similar data processing device. In embodiments, comparing as carried out in the context of the evaluation of the validated results comprises classifying the validated results in terms of similarity/dissimilarity to the reference set. Thus, an algorithm like “the MisMatch Match (MMM) approach” can be used. In this method, the step of analyzing comprises a step of correlating at least two vectors, wherein at least one of the at least two vectors is subjected to a shrinkage process for the components of the vector, taking into account the reliability of the component.

In the methods described herein, any suitable means may be employed for measuring the response of the cell or the population of cells in vivo.

In some embodiments, the assaying comprises measuring (i) growth rate and/or (ii) durability of cell growth and/or (iii) durability of cell response in the cell or population of cells. In some embodiments, the assaying further comprises (i) growth rate and (ii) durability of cell growth and (iii) durability of cell response. In some embodiments, the assaying further comprises using a real-time quantitative live-cell analysis platform. In some embodiments using one or more restimulation cycles.

In some embodiments, the assaying comprises measuring bulk cytokine production for a panel of cytokines in the cell or the population of cells. In some embodiments, the assaying further comprises using a multiplex cytokine detection technique, an electrochemiluminescence (ECL) detection technique and/or ELISA.

In some embodiments, the assaying comprises conducting single cell cytokine profiling on the cell or the population of cells. In some embodiments, the assaying further comprises using a multiplex proteomics assay.

In some embodiments, the assaying comprises conducting gene expression profiling on the cell or the population of cells to determine activation level (i) in a pre-modification resting state of the cell or the population of cells and/or (ii) in a post-modification resting state of the cell or the population of cells and/or (iii) in a post-modification activated state of the cell or the population of cells and/or (iv) in a post tumor challenge exhausted state. In some embodiments, the assaying further comprises conducting gene expression profiling on the cell or the population of cells to determine activation level in a post-modification resting state of the cell or the population of cells. In some embodiments, the assaying further comprises using direct digital detection of mRNA molecules of interest. In some embodiments, the assaying further comprises conducting gene expression profiling to determine activation level (i) in a pre-modification resting state of the cell or the population of cells and (ii) in a post-modification resting state of the cell or the population of cells and (iii) in a post-modification activated state of the cell or the population of cells and (iv) in a post tumor challenge exhausted state.

In some embodiments, the assaying comprises measuring the response of the cell or the population of cells in vivo. In some embodiments, the assaying further comprises transplanting the cell or the population of cells into an allogeneic host and monitoring for cell activity. In some embodiments, the assaying further comprises transplanting the cell or the population of cells into an allogeneic host and monitoring for cell escape from the host immune system. In some embodiments, the assaying further comprises transplanting the cell or the population of cells into an allogeneic host and monitoring for cell growth. In some embodiments, the assaying further comprises transplanting the cell or the population of cells into an allogeneic host and monitoring for cell rejection. In some embodiments, the assaying further comprises transplanting the cell or the population of cells into an allogeneic host and monitoring for cell survival. In some embodiments, the assaying further comprises transplanting the cell or the population of cells and a target cell or a population of target cells into an allogeneic host and monitoring for cell activity. In some embodiments, the assaying further comprises transplanting the cell or the population of cells and a target cell or a population of target cells into an allogeneic host and monitoring for cell escape from the host immune system. In some embodiments, the assaying further comprises transplanting the cell or the population of cells and a target cell or a population of target cells into an allogeneic host and monitoring for cell growth. In some embodiments, the assaying further comprises optionally transplanting the cell or the population of cells and a target cell or a population of target cells into an allogeneic host and monitoring for cell rejection. In some embodiments the assaying further comprises transplanting the cell or the population of cells and a target cell or a population of target cells into an allogeneic host and monitoring for cell survival.

In some embodiments, the assaying further comprises transplanting the cell or the population of cells into an allogeneic host and monitoring for teratoma formation.

In some embodiments, the assaying comprises measuring the response of a host to the cell or the population of cells. In some embodiments, the assaying further comprises transplanting the cell or the population of cells into an allogeneic host, obtaining immune cells from the allogeneic host, and determining an activation state of the immune cells from the allogeneic host. In some embodiments, the assaying further comprises transplanting the cell or the population of cells into an allogeneic host, obtaining a blood sample from the allogeneic host, and determining a humoral response of the allogeneic host.

In some embodiments, any or all or any combination of the assays are contemplated.

In some embodiments, step (a) of the assaying comprises providing a reference value for the measured (i) growth rate and/or (ii) durability of cell growth and/or (iii) durability of cell response and determining whether the measured value(s)is (i) above the reference value; (ii) at the reference value or (iii) below the reference value.

In some embodiments, step (b) of the assaying comprises providing a reference value for the bulk cytokine production and determining whether said measured value(s) is (i) above the reference value; (ii) at the reference value or (iii) below the reference value.

In some embodiments, step (c) of the assaying comprises calculating a polyfunctionality strength index (PSi) and/or a multifunctional strength index (MSi) from the single cell cytokine profiling, providing a reference value for the PSi and/or MSi and determining whether the calculated value(s) is (i) above the reference value; (ii) at the reference value or (iii) below the reference value, wherein PSi is defined as the signal intensity for cells that produce 2+ cytokines in the population of cells and MSi is defined as the percentage of cells in the population of cells that produce 4+ cytokines.

In some embodiments, the assaying comprises (i) identifying cells or the populations of cells determined as being at the reference value or above the reference value in any one of steps (a) to (c) and (ii) preparing a signature gene expression profile based on the gene expression profiles of the populations of cells identified in (i).

In some embodiments, step (d) of the assaying comprises providing a reference signature gene expression profile and comparing to the gene expression profile of the population of cells. In some embodiments, the reference signature gene expression profile is an average profile or a normal profile.

In some embodiments, step (e) of the assaying comprises (i) monitoring cell survival by bioluminescence imaging (BLI), (ii) determining successful engraftment by determining the surface expression of biomarkers; and/or determining the median area under the curve value (AUC) for the cell growth of the cell or the population of cells or the target cell or the population of target cells.

In some embodiments, the method further comprises identifying the cell or the population of cells or considering when identifying the cell or the population of cells as suitable for administration to a subject or suitable for making a cell therapy product if the cell or the population of cells is determined as being above the reference value or at the reference value in any of steps (a) to (c).

In some embodiments, the method further comprises identifying the cell or the population of cells or considering when identifying the cell or the population of cells as suitable for administration to a subject or suitable for making a cell therapy product if the gene expression profile of the cell or the population of cells is sufficiently similar to the reference signature gene expression profile. In some embodiments sufficiently similar is at least 60% similar, 70% similar, 80% similar, 90% similar or 99% similar.

In some embodiments, the method further comprises identifying the cell or the population of cells or considering when identifying the cell or the population of cells as suitable for administration to a subject or suitable for making a cell therapy product product if (i) the AUC is less than about 20 or between about 20 and 100 at high dose, or (ii) the AUC is less than about 11000 at low dose.

In some embodiments step (a) of the assaying comprises a serial challenge of the cell or the population of cells with a target cell or a population of target cells, optionally wherein the target cell or the population of target cells comprise a tumor cell or a population of tumor cells.

In some embodiments, an effector to target (E:T) ratio of about 1:8 or lower, such as 1:18, is used for the measuring using a real-time quantitative live-cell analysis platform in step (a). In some embodiments the E:T ratio is about 1:8, about 1:9, about 1:10, about 1:12, about 1:15, about 1:16, about 1:18 or about 1:20. In some embodiments the E:T ratio is about 1:8.

In some embodiments, the cell or the population of cells is identified or is considered when identifying the cell or the population of cells as suitable for administration to a subject or suitable for making a cell therapy product if the growth in each one or more restimulation cycles in step (a) is higher than a reference value of growth.

In some embodiments, the cell or the population of cells is identified or is considered when identifying the cell or the population of cells as suitable for administration to a subject or suitable for making a cell therapy product if the growth in each one or more restimulation cycles in step (a) is higher than about 0.2 fold more than a reference value of growth. In some embodiments, the growth in each one or more restimulation cycles in step (a) is higher than 4 fold.

In some embodiments, the cell or the population of cells is identified or is considered when identifying the cell or the population of cells as suitable for administration to a subject or suitable for making a cell therapy product if the durability of cell growth in step (a) is higher than a reference value of durability of cell growth.

In some embodiments, the cell or the population of cells is identified or is considered when identifying the cell or the population of cells as suitable for administration to a subject or suitable for making a cell therapy product if the durability of cell growth in step (a) is at least about 5% higher than a reference value of durability of cell growth In some embodiments the durability of cell growth in step (a) is at least 10% higher than a reference value of durability of cell growth.

In some embodiments, the cell or the population of cells is identified or is considered when identifying the cell or the population of cells as suitable for administration to a subject or suitable for making a cell therapy product if the durability of cell growth in step (a) is 0 or higher when durability of cell growth is measured as a slope of the fold change of the cell or the population of cells over a course of the serial challenge. In some embodiments, the assaying is performed using an IncuCyte assay.

In some embodiments, the cell or the population of cells is identified or is considered when identifying the cell or the population of cells as suitable for making a cell therapy product if the durability of cell response in step (a) is higher than a reference durability of cell response.

In some embodiments, the cell or the population of cells is identified or is considered when identifying the cell or the population of cells as suitable for administration to a subject or suitable for making a cell therapy product if the durability of cell response in step (a) is 0 or lower when durability of cell response is measured as a slope of the fold change of the target cell or the population of target cells over a course of the serial challenge, optionally wherein the assaying is performed using an IncuCyte assay.

In some embodiments, the panel of cytokines measured in step (b) comprises GM-CSF, GzmA, GzmB, IFN-g, TNF-a, IL-2, IL-6, IL-17A, IL-1b, IL-1RA, or any combinations thereof. In some embodiments, the panel of cytokines measured in step (b) comprises IL-17A. In some further embodiments, the panel of cytokines measured in step (b) further comprises at least one of GM-CSF, GzmA, GzmB, IFN-g, TNF-a, IL-2, IL-6, IL-1b, or IL-IRA.

In some embodiments, an E:T ratio of about 1:1, about 1:2, about 1:3, about 1:4, about 1:5, about 1:6, about 1:7, about 1:8, about 1:9, about 1:10, about 1:12, about 1:15, about 1:16, or about 1:20 is used for the measuring cytokine production in step (b). In some embodiments, an E:T ratio of 1:2 is used for the measuring of cytokine production in step (b).

In some embodiments, the cell or the population of cells is identified or is considered when identifying the cell or the population of cells as suitable for administration to a subject or suitable for making a cell therapy product if the measured cytokine production in step (b) is higher than the median of the average cytokine production values calculated for the more than one reference cell population, optionally about 5% or more.

In some embodiments, the cell or the population of cells is identified or is considered when identifying the cell or the population of cells as suitable for administration to a subject or suitable for making a cell therapy product if the measured cytokine production in step (b) is at least about 10% higher than the median of the average cytokine production values calculated for the more than one reference cell population.

In some embodiments, the bulk cytokine production in step (b) is measured at an E:T ratio of 1:2 and the cell or the population of cells is identified or is considered when identifying the cell or the population of cells as suitable for administration to a subject or suitable for making a cell therapy product if bulk cytokine production is measured as about 1.0 pg per cell or higher, such as about 1.5 pg per cell or higher, or about 1.9 pg per cell or higher.

In some embodiments, a polyfunctionality strength index (PSi) is defined from the single cell cytokine profiling in step (c) based on the signal intensity for cells that produce 2+ cytokines in the population of cells.

In some embodiments, the cell or the population of cells is identified or is considered when identifying the cell or the population of cells as suitable for administration to a subject or suitable for making a cell therapy product if the polyfunctionality strength index (PSi) in step (c) is higher than a reference value.

In some embodiments, the cell or the population of cells is identified or is considered when identifying the cell or the population of cells as suitable for administration to a subject or suitable for making a cell therapy product if the polyfunctionality strength index (PSi) in step (c) is 90 or above, such as 100 or above, or 150 or above, or 200 or above.

In some embodiments, the cell or the population of cells is identified or is considered when identifying the cell or the population of cells as suitable for administration to a subject or suitable for making a cell therapy product if the polyfunctionality strength index (PSi) in step (c) is higher than the median polyfunctionality strength index (PSi) of more than one reference cell population. In some embodiments, the polyfunctionality strength index (PSi) in step (c) is 5% or more higher than the median polyfunctionality strength index (PSi) of more than one reference cell population.

In some embodiments, the cell or the population of cells is identified or is considered when identifying the cell or the population of cells as suitable for administration to a subject or suitable for making a cell therapy product if the polyfunctionality strength index (PSi) in step (c) is at least 10% higher than the median polyfunctionality strength index (PSi) of more than one reference cell population.

In some embodiments, the cell or the population of cells is identified or is considered when identifying the cell or the population of cells as suitable for administration to a subject or suitable for making a cell therapy product if the polyfunctionality strength index (PSi) in step (c) is at least 15% higher than the median polyfunctionality strength index (PSi) of more than one reference cell population.

In some embodiments, a multi-functionality strength index (MSi) is defined from the single cell cytokine profiling in step (c) for the percentage of cells in the population of cells that produce 4+ cytokines.

In some embodiments, the cell or the population of cells is identified or is considered when identifying the cell or the population of cells as suitable for administration to a subject or suitable for making a cell therapy product if the multi-functionality strength index (MSi) in step (c) is higher than the reference multi-functionality strength index (MSi).

In some embodiments, the cell or the population of cells is identified or is considered when identifying the cell or population of cells as suitable for administration to a subject or suitable for making a cell therapy product if the multi-functionality strength index (MSi) in step (c) is 0.1% or higher, such as 0.2% or higher or 0.3% or higher.

In some embodiments, the cell or the population of cells is identified or is considered when identifying the cell or the population of cells as suitable for administration to a subject or suitable for making a cell therapy product if, in the pre-modification resting state, the cell or cell population
i. has a less NK-like signature than the reference signature gene expression profile. In some embodiments, the NK-like signature comprises expression of CD160, GNLY, GZMH, KLRK1, NKG7, CD56, KIR3RD and/or KLRD1;
ii. has a less activated T cell state signature than the reference signature gene expression profile. In some embodiments, the activated cell state signature comprises expression of FASLG, GZMB, Il2RB. Il12RB, TIGIT, Tim-3, GITR and/or CD38;
iii. has decreased expression of negative regulators of viral mRNA translation than the reference signature gene expression profile. In some embodiments, the negative regulators of viral mRNA translation comprise IFIT1, IFIT3, OASL, OAS1 and/or OAS3;
iv. has a greater naïve (TN) and/or central memory (CM) T cell phenotype signature than the reference signature gene expression profile. In some embodiments, the cell phenotype comprises expression of CD9 and/or PEACAM(CD31); and/or
v. has greater TCR clonality signature differences than the reference signature gene expression profile. In some embodiments, the clonality signature differences comprise higher levels of TRBV28-public canonical MR1-restricted cells/mucosal-associated invariant (MAIT) cells.

In some embodiments, the cell or the population of cells is identified or is considered when identifying the cell or the population of cells as suitable for administration to a subject or suitable for making a cell therapy product if, in the pre-modification resting state, the cell or cell population has a less NK-like signature than the reference gene expression profile. In some embodiments, the NK-like signature comprises expression of CD160, GNLY, GZMH, KLRK1, NKG7, CD56, KIR3RD and/or KLRD1.

In some embodiments, the cell or the population of cells is identified or is considered when identifying the cell or the population of cells as suitable for administration to a subject or suitable for making a cell therapy product if, in the pre-modification resting state, the cell or cell population has a less activated cell state than the reference gene expression profile. In some embodiments, the activated cell state comprises expression of FASLG, GZMB, Il2RB, Il12RB, TIGIT, Tim-3, GITR and/or CD38.

In some embodiments, the cell or the population of cells is identified or is considered when identifying the cell or the population of cells as suitable for administration to a subject subject or suitable for making a cell therapy product if, in the pre-modification resting state, the cell or cell population has decreased expression of negative regulators of viral mRNA translation than the reference gene expression profile. In some embodiments, the negative regulators of viral mRNA translation comprise IFIT1, IFIT3, OASL, OAS1 and/or OAS3.

In some embodiments, the cell or the population of cells is identified or is considered when identifying the cell or the population of cells as suitable for administration to a subject subject or suitable for making a cell therapy product if, in the pre-modification resting state, the cell or cell population has a greater naïve (TN) and/or central memory (CM) cell phenotype than the reference gene expression profile. In some embodiments, the cell phenotype comprises expression of CD9 and/or PEACAM(CD31).

In some embodiments, the cell or the population of cells is identified or is considered when identifying the cell or the population of cells as suitable for administration to a subject subject or suitable for making a cell therapy product if, in the pre-modification resting state, the cell or cell population has greater clonality differences than the reference gene expression profile. In some embodiments, the clonality differences comprise higher levels of TRBV28-public canonical MR1-restricted cells/mucosal-associated invariant (MAIT) cells.

In some embodiments, the cell or the population of cells is identified or is considered when identifying the cell or the population of cells as suitable for administration to a subject or suitable for making a cell therapy product if, in the post-modification resting state, the cell or cell population
i. has reduced expression of Tb2/rc2 signature genes than the reference signature gene expression profile. In some embodiments, the Th2/Tc2 signature comprises expression of IL4, IL5 and/or IL13;
ii. has a reduced naïve (TN) and/or central memory (CM) T cell phenotype signature than the reference signature gene expression profile. In some embodiments, the cell phenotype signature comprises expression of CD9, CD45RA and/or PEACAM(CD31);
iii. has greater expression of Th1/Tc1 signature genes than the reference signature gene expression profile. In some embodiments, the Th1/Tc1 signature comprises expression of IRF8, IRF1 and/or IFNG;
iv. has greater expression of Th17 signature genes than the reference signature gene expression profile. In some embodiments, the Th17 signature comprises expression of IL22 and/or IL26; and/or
v. has greater TCR diversity signature differences than the reference signature gene expression profile. In some embodiments, the diversity signature differences comprise higher levels of TRBV28-public canonical MR1-restricted cells/mucosal-associated invariant (MAIT) cells.

In some embodiments, the cell or the population of cells is identified or is considered when identifying the cell or the population of cells as suitable for administration to a subject or suitable for making a cell therapy product if, in the post-modification resting state, the cell or cell population has reduced expression of Th2/Tc2 signature genes than the reference signature gene expression profile. In some embodiments, the Th2/Tc2 signature comprises expression of IL4, IL5 and/or IL 13.

In some embodiments, the cell or the population of cells is identified or is considered when identifying the cell or the population of cells as suitable for administration to a subject or suitable for making a cell therapy product if, in the post-modification resting state, the cell or cell population has a reduced naïve (TN) and/or central memory (CM) T cell phenotype signature than the reference gene expression profile. In some embodiments, the cell phenotype signature comprises expression of CD9, CD45RA and/or PEACAM(CD31).

In some embodiments, the cell or the population of cells is identified or is considered when identifying the cell or the population of cells as suitable for administration to a subject or suitable for making a cell therapy product if, in the post-modification resting state, the cell or cell population has greater expression of Th1/Tc1 signature genes than the reference signature gene expression profile. In some embodiments, the Th1/Tc1 signature comprises expression of IRF8, IRF1 and/or IFNG.

In some embodiments, the cell or the population of cells is identified or is considered when identifying the cell or the population of cells as suitable for administration to a subject or suitable for making a cell therapy product if, in the post-modification resting state, the cell or cell population has greater expression of Th17 signature genes than the reference signature gene expression profile. In some embodiments, the Th17 signature comprises expression of IL22 and/or IL26.

In some embodiments, the cell or the population of cells is identified or is considered when identifying the cell or the population of cells as suitable for administration to a subject or suitable for making a cell therapy product if, in the post-modification resting state, the cell or the cell population has greater diversity signature differences than the reference gene expression profile. In some embodiments, the diversity differences comprise higher levels of TRBV28-public canonical MR1-restricted cells/mucosal-associated invariant (MAIT) cells.

In some embodiments, the cell or the population of cells is identified or is considered when identifying the cell or the population of the cells as suitable for administration to a subject or suitable for making a cell therapy product if, in the post-modification activated state, the cell or cell population
i. has reduced expression of Th2/Tc2 signature genes than the reference signature gene expression profile. In some embodiments, the Th2/Tc2 signature comprises expression of IL5.
ii. has a reduced naïve (TN) and/or central memory (CM) cell phenotype than the reference signature gene expression profile. In some embodiments, the cell phenotype comprises expression of PEACAM(CD31);
iii. has greater expression of Th1/Tc1 signature genes than the reference signature gene expression profile. In some embodiments, the Th1/Tc1 signature comprises expression of IRF1 and/or IFNG;
iv. has greater expression of Th17/Tc17 signature genes than the reference signature gene expression profile. In some embodiments, the Th17/Tc17 signature comprises expression of IL22, Il17F and/or IL26;
v. has greater diversity differences than the reference signature gene expression profile. In some embodiments, the diversity differences comprise higher levels of TRBV28, KLRB1, MAIT cells; and/or
vi. has greater cell activation/stimulation than the reference signature gene expression profile. In some embodiments, the cell activation comprises expression of ICOS, OX40, Lag3, GITR, VISTA, CD40L, CTLA4 and/or 4-1BB.

In some embodiments, the cell or the population of cells is identified or is considered when identifying the cell or population of cells as suitable for administration to a subject or suitable for making a cell therapy product if, in the post-modification activated state, the cell or cell population has reduced expression of Th2/Tc2 signature genes than the reference signature gene expression profile. In some embodiments, the Th2/Tc2 signature comprises expression of IL5.

In some embodiments, the cell or the population of cells is identified or is considered when identifying the cell or population of cells as suitable for administration to a subject or suitable for making a cell therapy product if, in the post-modification activated state, the cell or cell population has a reduced naïve (TN) and/or central memory (CM) cell phenotype signature than the reference gene expression profile. In some embodiments, the cell phenotype comprises expression of PEACAM(CD31).

In some embodiments, the cell or the population of cells is identified or is considered when identifying the cell or population of cells as suitable for administration to a subject or suitable for making a cell therapy product if, in the post-modification activated state, the cell or cell population has greater expression of Th1/Tc1 signature genes than the reference signature gene expression profile. In some embodiments, the Th1/Tc1 signature comprises expression of IRF1 and/or IFNG.

In some embodiments, the cell or the population of cells is identified or is considered when identifying the cell or population of cells as suitable for administration to a subject or suitable for making a cell therapy product if, in the post-modification activated state, the cell or cell population has greater expression of Th17/Tc17 signature genes than the reference signature gene expression profile. In some embodiments, the Th17/Tc17 signature comprises expression of IL22, Il17F and/or IL26.

In some embodiments, the cell or the population of cells is identified or is considered when identifying the cell or population of cells as suitable for administration to a subject or suitable for making a cell therapy product if, in the post-modification activated state, the cell or cell population has greater diversity differences than the reference signature gene expression profile. In some embodiments, the diversity differences comprise higher levels of TRBV28, KLRB1, MAIT cells.

In some embodiments, the cell or the population of cells is identified or is considered when identifying the cell or population of cells as suitable for administration to a subject or suitable for making a cell therapy product if, in the post-modification activated state, the cell or cell population has greater cell activation/stimulation than the reference signature gene expression profile. In some embodiments, the cell activation comprises expression of ICOS, OX40, Lag3, GITR, VISTA, CD40L, CTLA4 and/or 4-1BB.

In some embodiments, the cell or the population of cells is identified or is considered when identifying the cell or the population of cells as suitable for administration to a subject or suitable for making a cell therapy product if, in the post-tumor challenge exhausted state, the cell or cell population
i. has reduced expression of CREB1 and/or SOCS4 (inhibitor of NFAT TF and TCR signalling) than the reference signature gene expression profile; and/or
ii. has greater expression of FOS (part of NFAT TF) and/or MTCP1 (enhancer of AKT signalling) than the reference signature gene expression profile.

In some embodiments, the cell or the population of cells is identified or is considered when identifying the cell or the population of cells as suitable for administration to a subject or suitable for making a cell therapy product if, in the post-tumor challenge exhausted state, the cell or cell population has reduced expression of CREB1 and/or SOCS4 (inhibitor of NFAT TF and TCR signalling) than the reference signature gene expression profile.

In some embodiments the cell or the population of cells is identified or is considered when identifying the cell or the population of cells as suitable for administration to a subject or suitable for making a cell therapy product if, in the post-tumor challenge exhausted state, the cell or cell population has greater expression of FOS (part of NFAT TF) and/or MTCP1 (enhancer of AKT signalling) than the reference signature gene expression profile.

In some embodiments of the method described above comprising steps a to f, the method comprises at least step (a). In some embodiments, the method comprises at least step (b). In some embodiments, the method comprises at least step (c). In some embodiments, the method comprises at least step (d). In some embodiments, the method comprises at least step (e). In some embodiments, the method comprises at least step (f). In some embodiments, the method comprises at least step (g). In some embodiments, the method comprises at least step (a) and step (b). In some embodiments, the method comprises at least step (a) and step (c). In some embodiments, the method comprises at least step (a) and step (d). In some embodiments, the method comprises at least step (a) and step (e). In some embodiments, the method comprises at least step (a) and step (f). In some embodiments, the method comprises at least step (a) and step (g). In some embodiments, the method comprises at least step (b) and step (c). In some embodiments, the method comprises at least step (b) and step (d). In some embodiments, the method comprises at least step (b) and step (e). In some embodiments, the method comprises at least step (b) and step (f). In some embodiments, the method comprises at least step (b) and step (g). In some embodiments, the method comprises at least step (c) and step (d). In some embodiments, the method comprises at least step (c) and step (e). In some embodiments, the method comprises at least step (c) and step (f). ). In some embodiments, the method comprises at least step (c) and step (g). In some embodiments, the method comprises at least step (d) and step (e). In some embodiments, the method comprises at least step (d) and step (f). In some embodiments, the method comprises at least step (d) and step (g). In some embodiments, the method comprises at least step (e) and step (f). In some embodiments, the method comprises at least step (e) and step (g). In some embodiments, the method comprises at least step (f) and step (g). In some embodiments, the method comprises at least step (a), step (b) and step (c). In some embodiments, the method comprises at least step (a), step (b) and step (d). In some embodiments, the method comprises at least step (a), step (b) and step (e). In some embodiments, the method comprises at least step (a), step (b) and step (f). In some embodiments, the method comprises at least step (a), step (b) and step (g). In some embodiments, the method comprises at least step (a), step (c) and step (d). In some embodiments, the method comprises at least step (a), step (c) and step (e). In some embodiments, the method comprises at least step (a), step (c) and step (f). In some embodiments, the method comprises at least step (a), step (c) and step (g). In some embodiments, the method comprises at least step (a), step (d) and step (e). In some embodiments, the method comprises at least step (a), step (d) and step (f). In some embodiments, the method comprises at least step (a), step (d) and step (g). In some embodiments, the method comprises at least step (a), step (e) and step (f). In some embodiments, the method comprises at least step (a), step (e) and step (g). In some embodiments, the method comprises at least step (b), step (c) and step (d). In some embodiments, the method comprises at least step (b), step (c) and step (e). In some embodiments, the method comprises at least step (b), step (c) and step (f). In some embodiments, the method comprises at least step (b), step (c) and step (g). In some embodiments, the method comprises at least step (b), step (d) and step (e). In some embodiments, the method comprises at least step (b), step (d) and step (f). In some embodiments, the method comprises at least step (b), step (d) and step (g). In some embodiments, the method comprises at least step (b), step (e) and step (f). In some embodiments, the method comprises at least step (b), step (e) and step (g). In some embodiments, the method comprises at least step (c), step (d) and step (e). In some embodiments, the method comprises at least step (c), step (d) and step (f). In some embodiments, the method comprises at least step (c), step (d) and step (g). In some embodiments, the method comprises at least step (c), step (e) and step (f). In some embodiments, the method comprises at least step (c), step (e) and step (g). In some embodiments, the method comprises at least step (d), step (e) and step (f). In some embodiments, the method comprises at least step (d), step (e) and step (g). In some embodiments, the method comprises at least step (d), step (f) and step (g). In some embodiments, the method comprises at least step (e), step (f) and step (g). In some embodiments, the method comprises at least step (a), step (b), step (c) and step (d). In some embodiments, the method comprises at least step (a), step (b), step (c) and step (e). In some embodiments, the method comprises at least step (a), step (b), step (c) and step (f). In some embodiments, the method comprises at least step (a), step (b), step (c) and step (g). In some embodiments, the method comprises at least step (a), step (b), step (d) and step (e). In some embodiments, the method comprises at least step (a), step (b), step (d) and step (f). In some embodiments, the method comprises at least step (a), step (b), step (d) and step (g). In some embodiments, the method comprises at least step (a), step (b), step (e) and step (f). In some embodiments, the method comprises at least step (a), step (b), step (e) and step (g). In some embodiments, the method comprises at least step (a), step (b), step (f) and step (g). In some embodiments, the method comprises at least step (a), step (c), step (d) and step (e). In some embodiments, the method comprises at least step (a), step (c), step (d) and step (f). In some embodiments, the method comprises at least step (a), step (c), step (d) and step (g). In some embodiments, the method comprises at least step (a), step (c), step (e) and step (f). In some embodiments, the method comprises at least step (a), step (c), step (e) and step (g). In some embodiments, the method comprises at least step (a), step (c), step (f) and step (g). In some embodiments, the method comprises at least step (a), step (d), step (e) and step (f). In some embodiments, the method comprises at least step (a), step (d), step (e) and step (g). In some embodiments, the method comprises at least step (a), step (d), step (f) and step (g). In some embodiments, the method comprises at least step (a), step (e), step (f) and step (g). In some embodiments, the method comprises at least step (b), step (c), step (d) and step (e). In some embodiments, the method comprises at least step (b), step (c), step (d) and step (f). In some embodiments, the method comprises at least step (b), step (c), step (d) and step (g). In some embodiments, the method comprises at least step (b), step (c), step (e) and step (f). In some embodiments, the method comprises at least step (b), step (c), step (e) and step (g). In some embodiments, the method comprises at least step (b), step (c), step (f) and step (g). In some embodiments, the method comprises at least step (b), step (d), step (e) and step (f). In some embodiments, the method comprises at least step (b), step (d), step (e) and step (g). In some embodiments, the method comprises at least step (b), step (d), step (f) and step (g). In some embodiments, the method comprises at least step (b), step (e), step (f) and step (g). In some embodiments, the method comprises at least step (c), step (d), step (e) and step (f). In some embodiments, the method comprises at least step (c), step (d), step (e) and step (g). In some embodiments, the method comprises at least step (c), step (d), step (f) and step (g). In some embodiments, the method comprises at least step (c), step (e), step (f) and step (g). In some embodiments, the method comprises at least step (d), step (e), step (f) and step (g). In some embodiments, the method comprises step (a), step (b), step (c), step (d) and step (e). In some embodiments, the method comprises step (a), step (b), step (c), step (d) and step (f). In some embodiments, the method comprises step (a), step (b), step (c), step (d) and step (g). In some embodiments, the method comprises step (a), step (b), step (c), step (e) and step (f). In some embodiments, the method comprises step (a), step (b), step (c), step (e) and step (g). In some embodiments, the method comprises step (a), step (b), step (c), step (f) and step (g). In some embodiments, the method comprises step (a), step (b), step (d), step (e) and step (f). In some embodiments, the method comprises step (a), step (b), step (d), step (e) and step (g). In some embodiments, the method comprises step (a), step (b), step (d), step (f) and step (g). In some embodiments, the method comprises step (a), step (b), step (e), step (f) and step (g). In some embodiments, the method comprises step (a), step (c), step (d), step (e) and step (f). In some embodiments, the method comprises step (a), step (c), step (d), step (e) and step (g). In some embodiments, the method comprises step (a), step (c), step (d), step (f) and step (g). In some embodiments, the method comprises step (a), step (c), step (e), step (f) and step (g). In some embodiments, the method comprises step (a), step (d), step (e), step (f) and step (g). In some embodiments, the method comprises step (b), step (c), step (d), step (e) and step (f). In some embodiments, the method comprises step (b), step (c), step (d), step (e) and step (g). In some embodiments, the method comprises step (b), step (c), step (d), step (f) and step (g). In some embodiments, the method comprises step (b), step (c), step (e), step (f) and step (g). In some embodiments, the method comprises step (b), step (d), step (e), step (f) and step (g). In some embodiments, the method comprises step (c), step (d), step (e), step (f) and step (g). In some embodiments, the method comprises step (a), step (b), step (c), step (d), step (e) and step (f). In some embodiments, the method comprises step (a), step (b), step (c), step (d), step (e) and step (g). In some embodiments, the method comprises step (a), step (b), step (c), step (d), step (f) and step (g). In some embodiments, the method comprises step (a), step (b), step (c), step (e), step (f) and step (g). In some embodiments, the method comprises step (a), step (b), step (d), step (e), step (f) and step (g). In some embodiments, the method comprises step (a), step (c), step (d), step (e), step (f) and step (g). In some embodiments, the method comprises step (b), step (c), step (d), step (e), step (f) and step (g). In some embodiments, the method comprises step (a), step (b), step (c), step (d), step (e), step (f) and step (g).

In some embodiments, the host is a mammal. In some embodiments, the host is murine. In some embodiments, the host is a humanized model. In some embodiments, the host is an allogeneic humanized immunodeficient mouse model, optionally wherein the host is an allogeneic humanized NSG-SGM3 mouse.

In some embodiments, the method further comprises administering the cell or the population of cells to a subject.

In some embodiments, the population includes cells with hypoimmune gene modifications (HIP cells) that exhibit reduced expression of one or more molecules of the MHC class I and/or MHC class II molecules and optionally also exhibit increased expression of at least one tolerogenic factor; and at least 30%, at least 40%, at least 50%, at least 60%, at least 70%, at least 80%, or at least 90% of cells in the population exhibit reduced expression of one or more molecules of the MHC class I and/or MHC class II molecules and optionally also exhibit increased expression of at least one tolerogenic factor.

In some embodiments, at least 5%, at least 10%, at least 20%, at least 30%, at least 40%, at least 50%, at least 60%, at least 70% of cells in the population of cells do not exhibit reduced expression of one or more molecules of the MHC class I and/or MHC class II molecules and optionally also do not exhibit increased expression of at least one tolerogenic factor.

In some embodiments, 30-90%, 30-80%, 30-70%, 30-60%, 30-50% or 40-50% of cells in the population of cells are HIP cells that exhibit reduced expression of one or more molecules of the MHC class I and/or MHC class II molecules and optionally increased expression of at least one tolerogenic factor.

In some embodiments, 70-100%, 80-100%, or 90-100% of cells in the population of cells express a CAR.

In some embodiments, the subject is in need of therapy. In some embodiments, the subject is a patient (i.e., known or suspected of having a disease, disorder or condition).

In some embodiments, the cellular deficiency is diabetes, cancer, vascularization disorders, ocular disease, thyroid disease, skin diseases, and liver diseases), a condition or disease associated with a vascular condition or disease; a vascular condition or disease; a condition or disease associated with autoimmune thyroiditis; autoimmune thyroiditis; a condition or disease associated with a liver disease; liver disease, In some embodiments the condition is cirrhosis of the liver. In some embodiments the condition is a condition or disease associated with a corneal disease; corneal disease. In some embodiments the condition is Fuchs dystrophy or congenital hereditary endothelial dystrophy). In some embodiments the condition is a condition or disease associated with a kidney disease; kidney disease; a disease associated with cancer; cancer. In some embodiments the condition is a B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, or bladder cancer); a condition or disease associated with a hematopoietic disease or disorder; a hematopoietic disease or disorder. In some embodiments the condition is a myelodysplasia, aplastic anemia, Fanconi anemia, paroxysmal nocturnal hemoglobinuria, Sickle cell disease, Diamond Blackfan anemia, Schachman Diamond disorder, Kostmann's syndrome, chronic granulomatous disease, adrenoleukodystrophy, leukocyte adhesion deficiency, hemophilia, thalassemia, beta-thalassemia, leukaemia such as acute lymphocytic leukemia (ALL), acute myelogenous (myeloid) leukemia (AML), adult lymphoblastic leukaemia, chronic lymphocytic leukemia (CLL), B-cell chronic lymphocytic leukemia (B-CLL), chronic myeloid leukemia (CML), juvenile chronic myelogenous leukemia (CML), and juvenile myelomonocytic leukemia (JMML), severe combined immunodeficiency disease (SCID), X-linked severe combined immunodeficiency, Wiskott-Aldrich syndrome (WAS), adenosine-deaminase (ADA) deficiency, chronic granulomatous disease, Chediak-Higashi syndrome, Hodgkin's lymphoma, non-Hodgkin's lymphoma (NHL) or AIDS); a condition or disease associated with leukemia or myeloma; leukemia; myeloma; a condition or disease associated with an autoimmune disease or condition; an autoimmune disease or condition. In some embodiments the condition is acute disseminated encephalomyelitis, acute hemorrhagic leukoencephalitis, In some embodiments the condition is Addison's disease. Agammaglobulinemia, Alopecia areata, amyotrophic lateral sclerosis, ankylosing spondylitis, antiphospholipid syndrome, antisynthetase syndrome, atopic allergy, autoimmune aplastic anemia, autoimmune cardiomyopathy, autoimmune enteropathy, autoimmune hemolytic anemia, autoimmune hepatitis, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticaria, autoimmune uveitis, Balo disease, Balo concentric sclerosis, Bechets syndrome, Berger's disease, Bickerstaff's encephalitis, Blau syndrome, bullous pemphigoid, cancer, Castleman's disease, celiac disease, chronic inflammatory demyelinating polyneuropathy, chronic recurrent multifocal osteomyelitis, Churg-Strauss syndrome, cicatricial pemphigoid, Cogan syndrome, cold agglutinin disease, complement component 2 deficiency, cranial arteritis, CREST syndrome, Crohn's disease, Cushing's syndrome, cutaneous leukocytoclastic angiitis, Dego's disease, Dercum's disease, dermatitis herpetiformis, dermatomyositis, diabetes mellitus type 1, diffuse cutaneous systemic sclerosis, Dressler's syndrome, discoid lupus erythematosus, eczema, enthesitis-related arthritis, eosinophilic fasciitis, eosinophilic gastroenteritis, epidermolysis bullosa acquisita, erythema nodosum, essential mixed cryoglobulinemia, Evan's syndrome, firodysplasia ossificans progressiva, fibrosing aveolitis, gastritis, gastrointestinal pemphigoid, giant cell arteritis, glomerulonephritis, goodpasture's syndrome, Grave's disease, Guillain-Barre syndrome (GBS), Hashimoto's encephalitis, Hashimoto's thyroiditis, hemolytic anaemia, Henoch-Schonlein purpura, herpes gestationis, hypogammaglobulinemia, idiopathic inflammatory demyelinating disease, idiopathic pulmonary fibrosis, idiopathic thrombocytopenic purpura, IgA nephropathy, inclusion body myositis, inflammatory demyelinating polyneuropathy, interstitial cystitis, juvenile idiopathic arthritis, juvenile rheumatoid arthritis, Kawasaki's disease, Lambert-Eaton myasthenic syndrome, leukocytoclastic vasculitis, lichen planus, lichen sclerosus, linear IgA disease (LAD), Lou Gehrig's disease, lupoid hepatitis, lupus erythematosus, Majeed syndrome, Meniere's disease, microscopic polyangiitis, Miller-Fisher syndrome, mixed connective tissue disease, morphea, Mucha-Habermann disease, multiple sclerosis, myasthenia gravis, myositis, neuromyelitis optica, neuromyotonia, ocular cicatricial pemphigoid, opsoclonus myoclonus syndrome, ord thyroiditis, palindromic rheumatism, paraneoplastic cerebellar degeneration, paroxysmal nocturnal hemoglobinuria (PNH), Parry Romberg syndrome, Parsonnage-Turner syndrome, pars planitis, pemphigus, pemphigus vulgaris, permicious anemia, perivenous encephalomyelitis, POEMS syndrome, polyarteritis nodosa, polymyalgia rheumatica, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restless leg syndrome, retroperitoneal fibrosis, rheumatoid arthritis, rheumatoid fever, sarcoidosis, Schmidt syndrome, Schnitzler syndrome, scleritis, scleroderma, Sjogren's syndrome, spondylarthropathy, Still's disease, stiff person syndrome, subacute bacterial endocarditis, Susac's syndrome, Sweet's syndrome, Sydenham chorea, sympathetic ophthalmia, Takayasu's arteritis, temporal arteritis. Tolosa-Hunt syndrome, transverse myelitis, ulcerative colitis, undifferentiated connective tissue disease, undifferentiated spondylarthropathy, vasculitis, vitiligo or Wegener's granulomatosis); a condition or disease associated with Parkinson's disease, Huntington disease, multiple sclerosis, a neurodegenerative disease or condition, attention deficit hyperactivity disorder (ADHD), Tourette Syndrome (TS), schizophrenia, psychosis, depression, a neuropsychiatric disorder stroke, or amyotrophic lateral sclerosis (ALS); Parkinson's disease; Huntington disease; multiple sclerosis; a neurodegenerative disease or condition; attention deficit hyperactivity disorder (ADHD); Tourette Syndrome (TS); schizophrenia, psychosis, depression; a neuropsychiatric disorder stroke; or amyotrophic lateral sclerosis (ALS).

Optional features of the cell or the population of cells as used in the methods described herein are described herein in further detail below. Corresponding cells per se and population of cells per se are also provided according to the disclosure.

In some embodiments, the cell or the population of cells is cryopreserved. In some embodiments, the cell or the population of cells is cells are thawed. In some embodiments, the functional assays are performed before freezing. In some embodiments, the functional assays are performed after freezing. In some embodiments, the method further comprises thawing the cells. In some embodiments, the cell or the population of cells is not cryopreserved.

In some embodiments, after thawing the at least 30%, at least 40%, at least 50%, at least 60%, at least 70%, at least 80%, or at least 90% of cells in the population that exhibit reduced expression of one or more molecules of the MHC class I and/or MHC class II molecules and optionally also exhibit increased expression of at least one tolerogenic factor are viable cells.

In some embodiments, after thawing the at least 30%, at least 40%, at least 50%, at least 60%, at least 70%, at least 80%, or at least 90% of cells in the population that exhibit reduced expression of one or more molecules of the MHC class I and/or MHC class II molecules and optionally also exhibit increased expression of at least one tolerogenic factor comprise a mixture of viable and non-viable cells.

In some embodiments, the said cell or the population of cells are unmodified. In some embodiments, the cell or the population of cells are modified.

Engineered cells provided herein are modified cells.

Cells provided herein are suitable for use in the methods described herein. In other words, a method as described herein may be carried out using cells provide herein.

In some embodiments, the unmodified cell or unmodified population of cells used in a profiling method described herein is to be modified e.g., post-profiling. In such embodiments, any of the modifications described herein for a modified cell or modified population of cells may be the modifications that are to be introduced into the unmodified cell or unmodified population of cells.

In some embodiments, the cell or the population of cells comprise a chimeric antigen receptor (CAR).

In some embodiments, the CAR comprises a signal peptide, an extracellular binding domain, a hinge domain, a transmembrane domain, an intracellular costimulatory domain, and/or an intracellular signaling domain. In some embodiments, the CAR comprises a CD5-specific CAR, a CD19-specific CAR, a CD20-specific CAR, a CD22-specific CAR, a CD23-specific CAR, a CD30-specific CAR, a CD33-specific CAR, CD38-specific CAR, a CD70-specific CAR, a CD123-specific CAR, a CD138-specific CAR, a Kappa, Lambda, B cell maturation agent (BCMA)-specific CAR, a G-protein coupled receptor family C group 5 member D (GPRC5D)-specific CAR, a CD123-specific CAR, a LeY-specific CAR, a NKG2D ligand-specific CAR, a WT1-specific CAR, a GD2-specific CAR, a HER2-specific CAR, a EGFR-specific CAR, a EGFRvIII-specific CAR, a B7H3-specific CAR, a PSMA-specific CAR, a PSCA-specific CAR, a CAIX-specific CAR, a CD171-specific CAR, a CEA-specific CAR, a CSPG4-specific CAR, a EPHA2-specific CAR, a FAP-specific CAR, a FRα-specific CAR, a IL-13Rα-specific CAR, a Mesothelin-specific CAR, a MUC1-specific CAR, a MUC16-specific CAR, a ROR1-specific CAR, a C-Met-specific CAR, a CD133-specific CAR, a Ep-CAM-specific CAR, a GPC3-specific CAR, a HPV16-E6-specific CAR, a IL13Ra2-specific CAR, a MAGEA3-specific CAR, a MAGEA4-specific CAR, a MART1-specific CAR, a NY-ESO-1-specific CAR, a VEGFR2-specific CAR, a α-Folate receptor-specific CAR, a CD24-specific CAR, a CD44v7/8-specific CAR, a EGP-2-specific CAR, a EGP-40-specific CAR, a erb-B2-specific CAR, a erb-B 2,3,4-specific CAR, a FBP-specific CAR, a Fetal acethylcholine e receptor-specific CAR, a GD2-specific CAR, a GD3-specific CAR, a HMW-MAA-specific CAR, a IL-11 Rα-specific CAR, a KDR-specific CAR, a Lewis Y-specific CAR, a L1-cell adhesion molecule-specific CAR, a MAGE-A1-specific CAR, a Oncofetal antigen (h5T4)-specific CAR, a TAG-72-specific CAR, or a CD19/CD22-bispecific CAR. In some embodiments, the CAR comprises a CD19-specific CAR, a CD20-specific CAR, a CD22-specific CAR, a CD38-specific CAR, a CD123-specific CAR, a CD138-specific CAR, a BCMA-specific CAR, or a CD19/CD22-bispecific CAR. In some embodiments, the CAR is specific for CD19. In some embodiments, the CAR is specific for CD22. In some embodiments, the CAR is a CD19/CD22-bispecific CAR. In some embodiments, the cell or population of cells are CAR-T cells. In some embodiments, the method further comprises determining expression of the CAR in the cells.

In some embodiments, the cell or the population of cells are hypoimmunogenic. In some embodiments, the cell or the population of cells has been modified to be hypoimmunogenic. In some embodiments, the method further comprises modifying the cell or the population of cells to be hypoimmunogenic.

In some embodiments, the evaluating predicted cell function is performed on the cell or the population of cells prior to any hypoimmunogenic modifications.

In some embodiments, the cell or the population of cells are cryopreserved prior to any hypoimmunogenic modifications and the evaluating predicted cell function is performed: before the cell or the population of cells is cryopreserved, and/or after the cell or the population of cells has been cryopreserved and thawed.

In some embodiments, the cell or the population of cells are stored prior to any hypoimmunogenic modifications and evaluating predicted cell function is performed: before the cell or the population of cells is stored, and/or ii) after the cell or the population of cells has been stored.

In some embodiments, the evaluating predicted cell function is performed on the cell or the population of cells following introduction of one or more hypoimmune gene modifications.

In some embodiments, the cell or the population of cells are cryopreserved following introduction of one or more hypoimmune gene modifications and the evaluating predicted cell function is performed: before the cell or the population of cells is cryopreserved, and/or after the cell or the population of cells has been cryopreserved and thawed.

In some embodiments, the cell or the population of cells are stored following introduction of one or more hypoimmune gene modifications and evaluating predicted cell function is performed: before the cell or the population of cells is stored, and/or after the cell or the population of cells has been stored.

In some embodiments, the evaluating predicted cell function is performed on the cell or the population of cells following introduction of all hypoimmune gene modifications.

In some embodiments, the cell or the population of cells are cryopreserved following introduction of all hypoimmune gene modifications and the evaluating predicted cell function is performed: before the cell or the population of cells is cryopreserved, and/or after the cell or the population of cells has been cryopreserved and thawed.

In some embodiments, the cell or the population of cells are stored following introduction of all hypoimmune gene modifications and evaluating predicted cell function is performed: before the cell or the population of cells is stored, and/or after the cell or the population of cells has been stored.

In some embodiments, the evaluating predicted cell function is performed before cell differentiation, after cell differentiation, or both before and after cell differentiation.

In some embodiments, the evaluating predicted cell function is performed on the cell or the population of cells prior to any differentiation.

In some embodiments, the cell or the population of cells is cryopreserved prior to any differentiation and the evaluating predicted cell function is performed: before the cell or the population of cells is cryopreserved, and/or after the cell or the population of cells has been cryopreserved and thawed.

In some embodiments, the cell or the population of cells are stored prior to any differentiation and evaluating predicted cell function is performed: before the cell or the population of cells is stored, and/or after the cell or the population of cells has been stored.

In some embodiments, the evaluating predicted cell function is performed after the cell or the population of cells have been differentiated.

In some embodiments, the cell or the population of cells is cryopreserved after differentiation and the evaluating predicted cell function is performed: before the cell or the population of cells is cryopreserved, and/or after the cell or the population of cells has been cryopreserved and thawed.

In some embodiments, the cell or the population of cells are stored after differentiation and evaluating predicted cell function is performed: before the cell or the population of cells is stored, and/or after the cell or the population of cells has been stored.

In some embodiments, the evaluating predicted cell function is performed after the cell or the population of cells have completed differentiation.

In some embodiments, the cell or the population of cells are cryopreserved after completed differentiation and the evaluating predicted cell function is performed: before the cell or the population of cells is cryopreserved, and/or after the cell or the population of cells has been cryopreserved and thawed.

In some embodiments, the cell or the population of cells are stored after completed differentiation and evaluating predicted cell function is performed: before the cell or the population of cells is stored, and/or after the cell or the population of cells has been stored.

In some embodiments, the cell or population of cells that are cryopreserved or have been cryopreserved comprise decreased expression of one or more MHC class I molecules and/or one or more MHC class II molecules.

In some embodiments, the cell or population of cells that are cryopreserved or have been cryopreserved comprise increased expression of one or more tolerogenic factors.

In some embodiments, the cell or population of cells that are cryopreserved or have been cryopreserved comprise decreased expression of one or more MHC class I molecules and increased expression of one or more tolerogenic factors.

In some embodiments, the cell or population of cells that are cryopreserved or have been cryopreserved comprise decreased expression of one or more MHC class II molecules and increased expression of one or more tolerogenic factors.

In some embodiments, the cell or population of cells that are cryopreserved or have been cryopreserved comprise decreased expression of one or more MHC class I molecules, one or more MHC class II molecules, and increased expression of one or more tolerogenic factors.

In some embodiments, the cell or population of cells that are cryopreserved or have been cryopreserved comprise undifferentiated cells.

In some embodiments, the cell or population of cells that are cryopreserved or have been cryopreserved comprise cells that have been differentiated to an intermediate cell type.

In some embodiments, the cell or population of cells that are cryopreserved or have been cryopreserved comprise cells that have been differentiated to a fully differentiated cell type.

In some embodiments, the at least one cell parameter comprises determining hypoimmunity of the cell or the population of cells (e.g., using the XCelligence cell proliferation assay).

In some embodiments, the at least one cell parameter comprises determining CD47 expression (e.g., using flow cytometry) (e.g., at least about 1×, about 2×, about 3×, about 4×, about 5×, or more CD47 expression over baseline). In some embodiments, exogenous CD47 expression (e.g., at least about 1× CD47 expression) restores baseline levels of CD47 expression.

In some embodiments, the hypoimmunogenic cells have reduced expression of B2M, CIITA and TCRalpha and increased expression of CD47. In some embodiments, the modifying comprises reducing expression of B2M, CIITA and TCRalpha and increasing expression of CD47.

In some embodiments, the cell or the population of cells comprise one or more modifications. In some embodiments, the cell or population of cells comprise one or more modifications that (i) reduce expression of one or more MHC class I molecules and/or one or more MHC class II molecules, and/or (ii) increase expression of one or more tolerogenic factors, wherein the reduced expression of (i) and the increased expression of (ii) is relative to a cell of the same cell type that does not comprise the modifications.

In some embodiments, the one or more modifications in (i) reduce expression of:
a. one or more MHC class I molecules
b. one or more MHC class II molecules; or
c. one or more MHC class I molecules and one or more MHC class II molecules.

In some embodiments, the one or more modifications in (i) reduce expression of one or more molecules selected from the group consisting of B2M, TAP I, NLRC5, CIITA, HLA-A, HLA-B, HLA-C, HLA-DP, HLA-DQ, HLA-DR, HLA-DM, HLA-DO, RFX5, RFXANK. RFXAP, NFY-A, NFY-B, NFY-C, and any combination thereof.

In some embodiments, the cell or the population of cells do not express one or more molecules selected from the group consisting of B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, and combinations thereof.

In some embodiments, the one or more modifications that increase expression comprise increased cell surface expression, and/or the one or more modifications that reduce expression comprise reduced cell surface expression.

In some embodiments, the one or more modifications in (i) reduce expression of one or more MHC class I molecules. In some embodiments, the one or more modifications in (i) reduce expression of B2M. In some embodiments, the one or more modifications in (i) reduce expression of HLA-A, HLA-B, and/or HLA-C. In some embodiments, the one or more modifications in (i) reduce expression of one or more MHC class II molecules. In some embodiments, the one or more modifications in (i) reduce expression of CIITA. In some embodiments, the one or more modifications in (i) reduce expression of HLA-DM, HLA-DO, HLA-DP. HLA-DQ, HLA-DR, RFX5, RFXANK, and/or RFXAP.

In some embodiments, the one or more tolerogenic factors comprise one or more tolerogenic factors selected from the group consisting of A20/TNFAIP3, C1-Inhibitor, CCL21, CCL22, CD16, CD16 Fc receptor, CD24, CD27, CD35, CD39, CD46, CD47, CD52, CD55, CD59, CD200, CR1, CTLA4-Ig, DUX4, FasL, H2-M3, HLA-C, HLA-E, HLA-E heavy chain, HLA-F, HLA-G, IDO1, IL-10, IL15-RF, IL-35, MANF, Mfge8, PD-L1, Serpinb9, and any combination thereof.

In some embodiments, the one or more tolerogenic factors comprise CD47. In some embodiments, the one or more tolerogenic factors comprise CCL22. In some embodiments, the one or more tolerogenic factors comprise CD16 or CD16 Fc receptor. In some embodiments, the one or more tolerogenic factors comprise CD24. In some embodiments, the one or more tolerogenic factors comprise CD39. In some embodiments, the one or more tolerogenic factors comprise CR1. In some embodiments, the one or more tolerogenic factors comprise CD52. In some embodiments, the one or more tolerogenic factors comprise CD55. In some embodiments, the one or more tolerogenic factors comprise CD200. In some embodiments, the one or more tolerogenic factors comprise CD200. In some embodiments, the one or more tolerogenic factors comprise DUX4. In some embodiments, the one or more tolerogenic factors comprise HLA-E. In some embodiments, the one or more tolerogenic factors comprise HLA-G. In some embodiments, the one or more tolerogenic factors comprise IDO1. In some embodiments, the one or more tolerogenic factors comprise IL15-RF. In some embodiments, the one or more tolerogenic factors comprise IL35. In some embodiments, the one or more tolerogenic factors comprise PD-L1. In some embodiments, the one or more tolerogenic factors comprise MANF. In some embodiments, the one or more tolerogenic factors comprise A20/TNAIP3. In some embodiments, the one or more tolerogenic factors comprise HLA-E and CD47.

In some embodiments, the one or more tolerogenic factors comprise two or more tolerogenic factors selected from the group consisting of CD47, CD46, and CD59. In some embodiments, the one or more tolerogenic factors comprise CD47, CD46, and CD59.

In some embodiments, the one or more tolerogenic factors comprise two or more tolerogenic factors selected from the group consisting of CD47 and CD39. In some embodiments, the one or more tolerogenic factors comprise CD47 and CD39.

In some embodiments, the one or more tolerogenic factors comprise two or more tolerogenic factors selected from the group consisting of CD47 and CCL22. In some embodiments, the one or more tolerogenic factors comprise CD47 and CCL22.

In some embodiments, the one or more tolerogenic factors comprise two or more tolerogenic factors selected from the group consisting of CD47, HLA-G and PD-L1. In some embodiments, the one or more tolerogenic factors comprise CD47 and PD-L1. 212. In some embodiments, the one or more tolerogenic factors comprise CD47, HLA-G and PD-L1.

In some embodiments, the one or more tolerogenic factors comprise two or more tolerogenic factors selected from the group consisting of CD24, CD47, and PD-L1. In some embodiments, the one or more tolerogenic factors comprise CD24, CD47, and PD-L1.

In some embodiments, the one or more tolerogenic factors comprise two or more tolerogenic factors selected from the group consisting of HLA-E, CD24. CD47, and PD-L1. In some embodiments, the one or more tolerogenic factors comprise HLA-E, CD24, CD47, and PD-L1.

In some embodiments, the one or more tolerogenic factors comprise two or more tolerogenic factors selected from the group consisting of CD46, CD55, CD59, and CR1. In some embodiments, the one or more tolerogenic factors comprise CD46, CD55, CD59, and CR1.

In some embodiments, wherein the one or more tolerogenic factors comprise two or more tolerogenic factors selected from the group consisting of HLA-E, CD46, CD55, CD59, and CR1. In some embodiments, the one or more tolerogenic factors comprise HLA-E, CD46, CD55, CD59, and CR1.

In some embodiments, the one or more tolerogenic factors comprise two or more tolerogenic factors selected from the group consisting of HLA-E, CD24, CD47, PD-L1, CD46, CD55, CD59, and CR1. In some embodiments, the one or more tolerogenic factors comprise HLA-E, CD24, CD47, PD-L1, CD46, CD55, CD59, and CR1.

In some embodiments, the one or more tolerogenic factors comprise HLA-E and PD-L1.

In some embodiments, the one or more tolerogenic factors comprise two or more tolerogenic factors selected from the group consisting of HLA-E, PD-L1, and A20TNFAIP. In some embodiments, the one or more tolerogenic factors comprise HLA-E, PD-L1, and A20/TNFAIP.

In some embodiments, the one or more tolerogenic factors comprise two or more tolerogenic factors selected from the group consisting of HLA-E, PD-L1, and MANF. In some embodiments, the one or more tolerogenic factors comprise HLA-E, PD-L1, and MANF.

In some embodiments, the one or more tolerogenic factors comprise two or more tolerogenic factors selected from the group consisting of HLA-E, PD-L1, A20/TNFAIP, and MANF. In some embodiments, the one or more tolerogenic factors comprise HLA-E, PD-L1, A20/TNFAIP, and MANF.

In one aspect, the present disclosure provides an engineered cell comprising one or more modifications that (i) reduce expression of one or more MHC class I molecules and one or more MHC class II molecules, and (ii) increase expression of CD47, wherein the reduced expression of (i) and the increased expression of (ii) is relative to a cell of the same cell type that does not comprise the modifications.

In some embodiments, the one or more modifications in (i) reduce expression of one or more molecules selected from the group consisting of B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, and any combination thereof.

In some embodiments, the one or more modifications in (i) reduce expression of B2M.

In some embodiments, the one or more modifications in (i) reduce expression of HLA-A, HLA-B, and/or HLA-C.

In some embodiments, the one or more modifications in (i) reduce expression of CIITA.

In some embodiments, the one or more modifications in (i) reduce expression of HLA-DP, HLA-DR, and/or HLA-DQ.

In some embodiments, the one or more additional tolerogenic factors comprise one or more tolerogenic factors selected from the group consisting of A20/TNFAIP3, C1-Inhibitor, CCL21, CCL22, CD16, CD16 Fc receptor, CD24, CD27, CD35, CD39, CD46, CD47, CD52, CD55, CD59, CD200, CR1, CTLA4-Ig, DUX4, FasL, H2-M3, HLA-C, HLA-E, HLA-E heavy chain, HLA-F, HLA-G, IDO1, IL-10, IL15-RF, IL-35, MANF, Mfge8, PD-L1. Serpinb9, and any combination thereof.

In some embodiments, wherein the one or more additional tolerogenic factors comprise CD47.

In some embodiments, wherein the cell or the population of cells further comprises one or more modifications that reduce expression of one or more additional molecules.

In some embodiments, the one or more additional molecules comprises B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, ABO, CADM1, CD58, CD38, CD142, CD155, CEACAM1, CTLA-4, FUT1, ICAM1, IRF1, MIC-A, MIC-B, NLGN4Y, PCDH1IY, PD-1, a protein that is involved in oxidative or ER stress, RHD, TRAC, TRB. In some embodiments, the protein that is involved in oxidative or ER stress is selected from the group consisting of TXNIP, PERK, IRE1α, and DJ-1 (PARK7).

In some embodiments, the one or more additional molecules comprise one or more Y chromosome proteins. In some embodiments, the one or more Y chromosome proteins comprise Protocadherin-11 Y-linked (PCDH11Y) and/or Neuroligin-4 Y-linked (NLGN4Y).

In some embodiments, the one or more additional molecules comprise one or more NK cell ligands. In some embodiments, the one or more NK cell ligands comprise MIC-A and/or MIC-B.

In some embodiments, the one or more additional molecules comprise one or more proteins involved in oxidative or ER stress. In some embodiments, the one or more proteins involved in oxidative or ER stress comprise thioredoxin-interacting protein (TXNIP), PKR-like ER kinase (PERK), inositol-requiring enzyme 1α (IRE1α), and/or DJ-1 (PARK7).

In some embodiments, the one or more additional molecules comprise one or more blood antigen proteins. In some embodiments, the one or more blood antigen proteins comprise ABO, FUT1 and/or RHD.

In some embodiments, the cell or the population of cells further comprise one or more modifications that reduce expression of B2M, TAP I, NLRC5, CIITA, HLA-A, HLA-B, HLA-C, HLA-DP, HLA-DQ, HLA-DR, HLA-DM, HLA-DO. RFX5, RFXANK, RFXAP, NFY-A, NFY-B, NFY-C, ABO, CADM1, CD58, CD38, CD142, CD155, CEACAM1, CTLA-4, FUT1, ICAM1, IRF1, MIC-A, MIC-B, NLGN4Y, PCDH11Y, PD-1, a protein that is involved in oxidative or ER stress, RHD, TRAC, TRB. In some embodiments, the protein that is involved in oxidative or ER stress is selected from the group consisting of TXNIP, PERK, IRE1α, and DJ-1 (PARK7).

In some embodiments, TRB is TRBC1, TRBC2, or TRBC1 and TRBC2.

In some embodiments, reduced expression comprises no cell surface expression or no detectable cell surface expression.

In some embodiments, reduced expression comprises reduced mRNA expression. In some embodiments, reduced expression comprises no detectable mRNA expression.

In some embodiments, reduced expression comprises reduced protein expression or reduced protein activity. In some embodiments, reduced expression comprises no detectable protein expression or protein activity.

In some embodiments, reduced expression comprises eliminating activity of a gene encoding or regulating the expression of i) the one or more MHC class I molecules and/or the one or more MHC class II molecules, or ii) the one or more additional molecules.

In some embodiments, reduced expression comprises inactivation or disruption of an allele of a gene encoding or regulating the expression of i) the one or more MHC class I molecules and/or the one or more MHC class II molecules, or ii) the one or more additional molecules.

In some embodiments, reduced expression comprises inactivation or disruption of both alleles of a gene encoding or regulating the expression of i) the one or more MHC class I molecules and/or the one or more MHC class II molecules, or ii) the one or more additional molecules.

In some embodiments, the one or more modifications to reduce expression comprises an indel in a gene encoding or regulating the expression of i) the one or more MHC class I molecules and/or the one or more MHC class II molecules, or ii) the one or more additional molecules.

In some embodiments, the one or more modifications to reduce expression comprises a frameshift mutation or a deletion of a contiguous stretch of genomic DNA of a gene encoding or regulating the expression of i) the one or more MHC class I molecules and/or the one or more MHC class II molecules, or ii) the one or more additional molecules.

In some embodiments, the one or more modifications to reduce expression comprises inactivation or disruption of all coding sequences of a gene encoding or regulating the expression of i) the one or more MHC class I molecules and/or the one or more MHC class II molecules, or ii) the one or more additional molecules.

In some embodiments, the one or more modifications to reduce expression comprises knocking out a gene encoding or regulating the expression of i) the one or more MHC class I molecules and/or the one or more MHC class II molecules, or ii) the one or more additional molecules.

In some embodiments, the cell or the population of cells comprise one or more modifications that:
a. reduce expression of MHC class I and/or MHC class II molecules;
b. increase expression of CD47; and
c. increase expression of CCL22.

In some embodiments, the cell or the population of cells comprise one or more modifications that:
a. reduce expression of MHC class I and/or MHC class II molecules;
b. increase expression of CD47; and
c. increase expression of CD39.

In some embodiments, the cell or the population of cells comprise one or more modifications that:
a. reduce expression of MHC class I and/or MHC class II molecules;
b. increase expression of CD47; and
c. increase expression of CD46 and CD59.

In some embodiments, the cell or the population of cells comprise one or more modifications that:
a. reduce expression of MHC class I and/or MHC class II molecules;
b. increase expression of CD47; and
c. increase expression of PD-L1.

In some embodiments, the cell or the population of cells comprise one or more modifications that:
a. reduce expression of MHC class I and/or MHC class II molecules;
b. increase expression of CD47; and
c. increase expression of HLA-G and PD-L1.

In some embodiments, the cell or the population of cells comprise one or more modifications that:
a. reduce expression of MHC class I and/or MHC class II molecules;
b. increase expression of CD47; and
c. reduced expression of CD142 (TF).

In some embodiments, the cell or the population of cells comprise one or more modifications that:
a. reduce expression of MHC class I and/or MHC class II molecules;
b. increase expression of CD47; and
c. reduced expression of MIC-A and/or MIC-B.

In some embodiments, the cell or the population of cells comprise one or more modifications that:
a. reduce expression of MHC class I and/or MHC class II molecules; and
b. increase expression of CD24.

In some embodiments, the cell or the population of cells comprise one or more modifications that:
a. reduce expression of MHC class I and/or MHC class II molecules; and
b. increase expression of CD200.

In some embodiments, the cell or the population of cells comprise one or more modifications that:
a. reduce expression of MHC class I and/or MHC class II molecules; and
b. increase expression of CD52.

In some embodiments, the cell or the population of cells comprise one or more modifications that:
a. reduce expression of MHC class I and/or MHC class II molecules; and
b. increase expression of DUX4.

In some embodiments, the cell or the population of cells comprise one or more modifications that:
a. reduce expression of MHC class I and/or MHC class II molecules; and
b. increase expression of IDO1.

In some embodiments, the cell or the population of cells comprise one or more modifications that:
a. reduce expression of MHC class I and/or MHC class II molecules; and
b. increase expression of IL-35.

In some embodiments, the cell or the population of cells comprise one or more modifications that:
a. reduce expression of MHC class I and/or MHC class II molecules; and
b. increase expression of PD-L1.

In some embodiments, the cell or the population of cells comprise one or more modifications that:
a. reduce expression of MHC class I and/or MHC class II molecules; and
b. increase expression of HLA-E.

In some embodiments, the cell or the population of cells comprise one or more modifications that:
a. reduce expression of MHC class I and/or MHC class II molecules; and
b. increase expression of HLA-G.

In some embodiments, the cell or the population of cells comprise one or more modifications that:
a. reduce expression of MHC class I and/or MHC class II molecules;
b. reduce expression of CD155; and
c. increase expression of HLA-E.

In some embodiments, the cell or the population of cells comprise one or more modifications that:
a. reduce expression of MHC class I molecules;
b. reduce expression of RFXANK;
c. increase expression of HLA-E.

In some embodiments, the cell or the population of cells comprise one or more modifications that:
a. reduce expression of MHC class I and/or MHC class II molecules;
b. reduce expression of MIC-A and/or MIC-B;
c. increase expression of one or more of CD47, CD24 and PD-L1; and
d. increase expression of CD46, CD55, CD59 and CR1.

In some embodiments, the cell or the population of cells comprise one or more modifications that:
a. reduce expression of MHC class I molecules;
b. reduce expression of MIC-A and/or MIC-B;
c. reduce expression of TXNIP; and
d. increase expression of PD-L1 and HLA-E.

In some embodiments, the modifications further increase expression of A20/TNFAIP3 and MANF.

In some embodiments, the one or more modifications that reduce expression of MHC class I and/or MHC class II molecules consist of one or more modifications that reduce expression of MHC class I molecules.

In some embodiments, the one or more modifications that reduce expression of MHC class I and/or MHC class II molecules consist of one or more modifications that reduce expression of MHC class II molecules.

In some embodiments, the one or more modifications that reduce expression of MHC class I and/or MHC class II molecules consist of one or more modifications that reduce expression of MHC class I molecules and MHC class II molecules.

In some embodiments, the increased expression comprises increased mRNA expression.

In some embodiments, the increased expression comprises increased protein expression or protein activity.

In some embodiments, the increased expression comprises increasing activity of a gene encoding or regulating the expression of i) the one or more tolerogenic factors, or ii) the one or more additional tolerogenic factors.

In some embodiments, the gene is an endogenous gene and the one or more modifications comprise one or more modifications of an endogenous promoter.

In some embodiments, the gene is an endogenous gene and the one or more modifications comprise introduction of a heterologous promoter.

In some embodiments, the heterologous promoter is selected from the group consisting of a CAG promoter, cytomegalovirus (CMV) promoter, EF1α promoter, EF1α short promoter, PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein Barr virus (EBV) promoter, and Rous sarcoma virus (RSV) promoter, and UBC promoter.

In some embodiments, the ecell or the population of cells comprise one or more transgenes.

In some embodiments, the one or more transgenes encode at least one of the one or more tolerogenic factors or the one or more additional tolerogenic factors.

In some embodiments, the one or more transgenes encode at least one of the one or more additional tolerogenic factors.

In some embodiments, the one or more transgenes encode one or more additional molecules.

In some embodiments, the one or more transgenes comprise one or more regulatory elements.

In some embodiments, the one or more transgenes are operably linked to the one or more regulatory elements.

In some embodiments, the one or more regulatory elements comprise one or more promoters, enhancers, introns, terminators, translation initiation signals, polyadenylation signals, replication elements, RNA processing and export elements, transposons, transposases, insulators, internal ribosome entry sites (IRES), 5′UTRs, 3′UTRs, mRNA 3′ end processing sequences, boundary elements, locus control regions (LCR), matrix attachment regions (MAR), recombination or cassette exchange sequences, linker sequences, secretion signals, resistance markers, anchoring peptides, localization signals, fusion tags, affinity tags, chaperonins, and proteases.

In some embodiments, the promoter is selected from the group consisting of a CAG promoter, cytomegalovirus (CMV) promoter, EF1α promoter, EF1α short promoter, PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein Barr virus (EBV) promoter, and Rous sarcoma virus (RSV) promoter, and UBC promoter.

In some embodiments, the cell or the population of cells comprise one or more vectors encoding the one or more transgenes.

In some embodiments, at least one of the one or more vectors is a multicistronic vector.

In some embodiments, the multicistronic vector encodes at least one of the one or more tolerogenic factors or the one or more additional tolerogenic factors.

In some embodiments, the multicistronic vector further encodes at least one of the one or more tolerogenic factors or the one or more additional tolerogenic factors.

In some embodiments, the multicistronic vector further encodes at least one of the one or more additional molecules.

In some embodiments, the one or more transgenes are separated by one or more linker sequences.

In some embodiments, the one or more linker sequences comprise an IRES sequence or a cleavable peptide sequence.

In some embodiments, the cleavable peptide sequence comprises a self-cleavable peptide, optionally a 2A peptide.

In some embodiments, the 2A peptide is selected from the group consisting of a F2A sequence, an E2A sequence, a P2A sequence, and a T2A sequence.

In some embodiments, the cleavable peptide sequence comprises a protease cleavable sequence or a chemically cleavable sequence.

In some embodiments, the one or more tolerogenic factors, the one or more additional tolerogenic factors, and/or the one or more additional molecules are operably linked to the same promoter.

In some embodiments, the promoter is a constitutive promoter.

In some embodiments, the promoter is selected from the group consisting of a CAG promoter, cytomegalovirus (CMV) promoter, EF1α promoter, EF1α short promoter, PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein Barr virus (EBV) promoter, and Rous sarcoma virus (RSV) promoter, and UBC promoter.

In some embodiments, the one or more additional molecules comprise a chimeric antigen receptor (CAR).

In some embodiments, the cell or the population of cells comprise a chimeric antigen receptor (CAR).

In some embodiments, the evaluating predicted cell function is performed on the cells following introduction of a CAR transgene modifications.

In some embodiments, the at least one cell parameter comprises determining the presence of a CAR transgene modification in the cell or the population of cells.

In some embodiments, the at least one cell parameter comprises determining CAR expression in the cell or the population of cells.

In some embodiments, the CAR comprises a signal peptide, an extracellular binding domain specific to CD19, a hinge domain, a transmembrane domain, an intracellular costimulatory domain, and/or an intracellular signaling domain.

In some embodiments, the CAR comprises a CD5-specific CAR, a CD19-specific CAR, a CD20-specific CAR, a CD22-specific CAR, a CD23-specific CAR, a CD30-specific CAR, a CD33-specific CAR, CD38-specific CAR, a CD70-specific CAR, a CD123-specific CAR, a CD138-specific CAR, a Kappa, Lambda, B cell maturation agent (BCMA)-specific CAR, a G-protein coupled receptor family C group 5 member D (GPRC5D)-specific CAR, a CD123-specific CAR, a LeY-specific CAR, a NKG2D ligand-specific CAR, a WT1-specific CAR, a GD2-specific CAR, a HER2-specific CAR, a EGFR-specific CAR, a EGFRvIII-specific CAR, a B7H3-specific CAR, a PSMA-specific CAR, a PSCA-specific CAR, a CAIX-specific CAR, a CD171-specific CAR, a CEA-specific CAR, a CSPG4-specific CAR, a EPHA2-specific CAR, a FAP-specific CAR, a FRα-specific CAR, a IL-13Rα-specific CAR, a Mesothelin-specific CAR, a MUC1-specific CAR, a MUC16-specific CAR, a ROR1-specific CAR, a C-Met-specific CAR, a CD133-specific CAR, a Ep-CAM-specific CAR, a GPC3-specific CAR, a HPV16-E6-specific CAR, a IL13Ra2-specific CAR, a MAGEA3-specific CAR, a MAGEA4-specific CAR, a MART1-specific CAR, a NY-ESO-1-specific CAR, a VEGFR2-specific CAR, a α-Folate receptor-specific CAR, a CD24-specific CAR, a CD44v7/8-specific CAR, a EGP-2-specific CAR, a EGP-40-specific CAR, a erb-B2-specific CAR, a erb-B 2,3,4-specific CAR, a FBP-specific CAR, a Fetal acethylcholine e receptor-specific CAR, a GD2-specific CAR, a GD3-specific CAR, a HMW-MAA-specific CAR, a IL-11Rα-specific CAR, a KDR-specific CAR, a Lewis Y-specific CAR, a L1-cell adhesion molecule-specific CAR, a MAGE-A1-specific CAR, a Oncofetal antigen (h5T4)-specific CAR, a TAG-72-specific CAR, or a CD19/CD22-bispecific CAR.

In some embodiments, the CAR comprises a CD19-specific CAR, a CD20-specific CAR, a CD22-specific CAR, a CD38-specific CAR, a CD123-specific CAR, a CD138-specific CAR, a BCMA-specific CAR, or a CD19/CD22-bispecific CAR.

In some embodiments, the CAR is specific for CD19.

In some embodiments, the CAR is specific for CD22.

In some embodiments, the CAR is a CD19/CD22-bispecific CAR.

In some embodiments, the cell or the population of cells are CAR-T cells.

In some embodiments, the method further comprises determining expression of the CAR in the cells.

In some embodiments, the one or more additional molecules comprise one or more safety switches.

In some embodiments, the method further comprises determining the presence of one or more safety switches in the cell or the population of cells.

In some embodiments, the at least one cell parameter comprises assaying safety switch activity in the cell or the population of cells.

In some embodiments, the evaluating predicted cell function is performed on the cells following introduction of safety switch modifications.

In some embodiments, the one or more safety switches are capable of controlled killing of the cell or the population of cells.

In some embodiments, the one or more safety switches induce controlled cell death in the presence of a drug or prodrug, or upon activation by a selective exogenous compound.

In some embodiments, the one or more safety switches comprise is an inducible protein capable of inducing apoptosis of the cell or the population of cells

In some embodiments, the inducible protein capable of inducing apoptosis of the cell or the population of cells is a caspase protein.

In some embodiments, the caspase protein is caspase 9.

In some embodiments, the one or more safety switches comprise one or more suicide genes.

In some embodiments, the one or more suicide genes are selected from the group consisting of cytosine deaminase (CyD), herpesvirus thymidine kinase (HSV-Tk), an inducible caspase 9 (iCaspase9), and rapamycin-activated caspase 9 (rapaCasp9).

In some embodiments, the safety switch is an “uncloaking” system wherein upon activation, cells downregulate expression of immunosuppressive factors and/or upregulate expression of immune signaling molecules thereby marking the cell for elimination by the host immune system.

In some embodiments, the at least one of the one or more transgenes are integrated into the genome of the cell or the population of cells.

In some embodiments, one or more transgenes are integrated into the genome of the cell or the population of cells.

In some embodiments, the method further comprises determining integration of at least one of the one or more transgenes into the genome of the cell or the population of cells.

In some embodiments, the evaluating predicted cell function is performed following introduction of one or more transgenes into the genome of the cell or the population of cells.

In some embodiments, the at least one cell parameter comprises determining transgene expression in the cell or the population of cells.

In some embodiments, the integration is by non-targeted insertion into the genome of the cell or the population of cells.

In some embodiments, the integration is by non-targeted insertion into the genome of the cell or the population of cells using a lentiviral vector.

In some embodiments, the integration is by targeted insertion into a target genomic locus of the cell or the population of cells.

In some embodiments, the targeted insertion is with homology-directed repair.

In some embodiments, the target genomic locus is selected from the group consisting of an albumin gene locus, an ABO gene locus, a B2M gene locus, a CIITA gene locus, a CCR5 gene locus, a CD142 gene locus, a CLYBL gene locus, a CXCR4 gene locus, an F3 gene locus, a FUT1 gene locus, an HMGB1 gene locus, a KDM5D gene locus, an LRP1 gene locus, a MIC-A gene locus, a MIC-B gene locus, a PPPIR12C (also known as AAVS1) gene locus, an RHD gene locus, a ROSA26 gene locus, a safe harbor gene locus, a SHS231 locus, a TAP1 gene locus, a TRAC gene locus, and a TRBC gene locus.

In some embodiments, the genome of the cell or the population of cells comprises one or more gene edits in one or more genes encoding the one or more molecules described herein having reduced expression.

In some embodiments, the cell or the population of cells comprises a genome editing complex.

In some embodiments, the genome editing complex comprises a genome targeting entity and a genome modifying entity.

In some embodiments, the genome targeting entity localizes the genome editing complex to the target locus, optionally wherein the genome targeting entity is a nucleic acid-guided targeting entity.

In some embodiments, the genome targeting entity is selected from the group consisting of a sequence specific nuclease, a nucleic acid programmable DNA binding protein, an RNA guided nuclease. RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZF) nucleic acid binding entity, a transcription activator-like effector (TALE) nucleic acid binding entity, a meganuclease, a Cas nuclease, a core Cas protein, a nuclease, a homing endonuclease, an endonuclease-deficient-Cas protein, an enzymatically inactive Cas protein, a CRISPR-associated transposase (CAST), a Type II or Type V Cas protein, or a functional portion thereof.

In some embodiments, the genome targeting entity is selected from the group consisting of Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9. SpCas9-HF1, HypaSpCas9, HeFSpCas9, and evoSpCas9 high-fidelity variants of SpCas9, SaCas9, NmeCas9, CjCas9. StCas9. TdCas9. LbCas12a, AsCas12a, AacCas12b, BhCas12b v4, TnpB, dCas (D10A), dCas (H840A), dCas13a, dCas13b, or a functional portion thereof.

In some embodiments, the genome modifying entity cleaves, deaminates, nicks, polymerizes, interrogates, integrates, cuts, unwinds, breaks, alters, methylates, demethylates, or otherwise destabilizes the target locus.

In some embodiments, the genome modifying entity comprises a recombinase, integrase, transposase, endonuclease, exonuclease, nickase, helicase, DNA polymerase, RNA polymerase, reverse transcriptase, deaminase, flippase, methylase, demethylase, acetylase, a nucleic acid modifying protein, an RNA modifying protein, a DNA modifying protein, an Argonaute protein, an epigenetic modifying protein, a histone modifying protein, or a functional portion thereof.

In some embodiments, the genome modifying entity selected from the group consisting of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, base editing, prime editing, a Programmable Addition via Site-specific Targeting Elements (PASTE), or a functional portion thereof.

In some embodiments, the genome modifying entity is selected from the group consisting of Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10). Cas12g, Cas12h, Cas12i, Cas12k (C2c5), Cas13, Cas13a (C2c2). Cas13b. Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7. SpCas9, eSpCas9. SpCas9-HF1, HvpaSpCas9. HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, FokI, dCas (D10A), dCas (H840A), dCas13a, dCas13b, a base editor, a prime editor (e.g., a target-primed reverse transcription (TPRT) editor), APOBEC1, cytidine deaminase, adenosine deaminase, uracil glycosylase inhibitor (UGI), adenine base editors (ABE), cytosine base editors (CBE), reverse transcriptase, serine integrase, recombinase, transposase, polymerase, adenine-to-thymine or “ATBE” (or thymine-to-adenine or “TABE”) transversion base editor, ten-eleven translocation methylcytosine dioxygenases (TETs), TET1, TET3, TET1CD, histone acetyltransferase p300, histone methyltransferase SMYD3, histone methyltransferase PRDM9, H3K79 methyltransferase DOT1L, transcriptional repressor, or a functional portion thereof.

In some embodiments, the genome targeting entity and the genome modifying entity are different domains of a single polypeptide.

In some embodiments, the genome editing entity and genome modifying entity are two different polypeptides that are operably linked together.

In some embodiments, the genome editing entity and genome modifying entity are two different polypeptides that are not linked together.

In some embodiments, the genome editing complex comprises a guide nucleic acid having a targeting domain that is complementary to at least one target locus, optionally wherein the guide nucleic acid is a guide RNA (gRNA).

In some embodiments, the one or more modifications are made by the genome editing complex.

In some embodiments, the one or more modifications made by the genome editing complex are made by a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a TnpB nuclease, a homing endonuclease, an endonuclease-deficient-Cas protein, an enzymatically inactive Cas protein, a CRISPR-associated transposase (CAST), a Type II or Type V Cas protein, base editing, prime editing, or a Programmable Addition via Site-specific Targeting Elements (PASTE).

In some embodiments, the one or more modifications made by the genome editing complex are made by Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, a CRISPR-associated transposase, base editing, prime editing, or Programmable Addition via Site-specific Targeting Elements (PASTE).

In some embodiments, the modifications made by the genome editing complex are made using a guide RNA (gRNA) having a targeting domain that is complementary to at least one target site.

In some embodiments, the cell or the population of cells are human cells or animal cells.

In some embodiments, the animal cells are porcine cells, bovine cells, or ovine cells.

In some embodiments, the cell or the population of cells are human cells.

In some embodiments, the cell or the population of cells are stem cells or progenitor cells.

In some embodiments, the cell or the population of cells are differentiated cells derived from the stem cells or progenitor cells.

In some embodiments, the cell or the population of cells are progenitor cells.

In some embodiments, the stem cell or progenitor cell is selected from the group consisting of an induced pluripotent stem cell, an embryonic stem cell, a hematopoietic stem cell, a mesenchymal stem cell, an endothelial stem cell, an epithelial stem cell, an adipose stem cell, a germline stem cell, a lung stem cell, a cord blood stem cell, a pluripotent stem cell (PSC), and a multipotent stem cell.

In some embodiments, the cell or the population of cells are stem cell derived cells.

In some embodiments, the cell or the population of cells are differentiated cells derived from pluripotent stem cells or progenies thereof.

In some embodiments, the cell or the population of cells are pluripotent stem cells.

In some embodiments, the cell or the population of cells are hematopoietic stem cells (HSCs).

In some embodiments, the cell or the population of cells are multipotent cells.

In some embodiments, the pluripotent stem cell is an induced pluripotent stem cell.

In some embodiments, the cell or the population of cells are differentiated cells derived from induced pluripotent stem cells.

In some embodiments, the cell or the population of cells are differentiated cells derived from embryonic stem cells.

In some embodiments, the evaluating predicted cell function is performed on the cells pre-differentiation.

In some embodiments, the cells are stem cells and the evaluating predicted cell function is performed on the cells following differentiation.

In some embodiments, the evaluating predicted cell function is performed before cell differentiation, after cell differentiation, or both before and after cell differentiation.

In some embodiments, the evaluating predicted cell function is performed on the cell or the population of cells prior to any differentiation.

In some embodiments, the cell or the population of cells is cryopreserved prior to any differentiation and the evaluating predicted cell function is performed: before the cell or the population of cells is cryopreserved, and/or after the cell or the population of cells has been cryopreserved and thawed.

In some embodiments, the cell or the population of cells are stored prior to any differentiation and evaluating predicted cell function is performed: before the cell or the population of cells is stored, and/or after the cell or the population of cells has been stored.

In some embodiments, the evaluating predicted cell function is performed after the cell or the population of cells have been differentiated.

In some embodiments, the cell or the population of cells is cryopreserved after differentiation and the evaluating predicted cell function is performed: before the cell or the population of cells is cryopreserved, and/or after the cell or the population of cells has been cryopreserved and thawed.

In some embodiments, the cell or the population of cells are stored after differentiation and evaluating predicted cell function is performed: before the cell or the population of cells is stored, and/or after the cell or the population of cells has been stored.

In some embodiments, the evaluating predicted cell function is performed after the cell or the population of cells have completed differentiation.

In some embodiments, the cell or the population of cells are cryopreserved after completed differentiation and the evaluating predicted cell function is performed: before the cell or the population of cells is cryopreserved, and/or after the cell or the population of cells has been cryopreserved and thawed.

In some embodiments, the cell or the population of cells are stored after completed differentiation and evaluating predicted cell function is performed: before the cell or the population of cells is stored, and/or after the cell or the population of cells has been stored.

In some embodiments, the cell or the population of cells are autologous.

In some embodiments, the cell or the population of cells are allogeneic.

In some embodiments, the cell or the population of cells are primary cells isolated from a single donor subject.

In some embodiments, the cell or the population of cells are primary cells isolated from more than one donors.

In some embodiments, the cell or the population of cells are derived from a single donor.

In some embodiments, the cell or the population of cells are derived from pooled donor cells obtained from more than one donor.

The method as described herein, wherein each donor subject is healthy or is not suspected of having a disease or condition at the time the donor sample is obtained from the individual donor.

In some embodiments, the cell or population of cells are immune cells.

In some embodiments, the cell or population of cells are selected from the group consisting of islet cells, beta islet cells, pancreatic islet cells, immune cells, B cells, T cells, natural killer (NK) cells, natural killer T (NKT) cells, macrophages, endothelial cells, muscle cells, cardiac muscle cells, smooth muscle cells, skeletal muscle cells, dopaminergic neurons, retinal pigmented epithelium cells (e.g., retinal pigmented epithelium (RPE) cells and thyroid cells), optic cells, hepatocytes, thyroid cells, skin cells, glial progenitor cells, neural cells (e.g., cerebral endothelial cells, dopaminergic neurons, glial cells, and hematopoietic stem cells (HSCS) cells), cardiac cells, stem cells, hematopoietic stem cells, induced pluripotent stem cells (iPSCs), mesenchymal stem cells (MSCs), embryonic stem cells (ESCs), pluripotent stem cells (PSCs), and blood cells.

In some embodiments, the cell or the population of cells are T cells.

In some embodiments, the cell or the population of cells are natural killer (NK) cells.

In some embodiments, the differentiated cells are NK or T cells.

In some embodiments, the at least one cell parameter is cell stability e.g., measured using a humming bird assay (PCR based), aCGH arrays, G-banding, and/or X-chromosome inactivation.

In some embodiments, the at least one cell parameter comprises genome sequencing (e.g., whole genome sequencing or targeted genome sequencing) or exome sequencing (e.g., whole exome sequencing or targeted exome sequencing).

In some embodiments, at least one cell parameter comprises genome sequencing or exome sequencing for screening for safety compromising mutations (e.g., ABCA10, ABCA12, ABCC9, ABL1, ABL2, ACVR1, AKAP9, AKT1, AKT2, AKT3, ALK, ANGPTL1, ANKRD26, APC, AR, ARAF, ARID1A, ARID1B, ASPH, ASXL1, ASXL2, ATM, ATR, ATRX, AURKA, AURKB, AXIN2, AXL, BABAM1, BAK1, BAP1, BARD1, BCL2, BCL2L11, BCOR, BCORL1, BCR, BTRC3, BLM, BMPR1A, BRAF, BRCA1, BRCA1&2 Sequencing, BRCA2, BRIP1, BRWD3, BTK, CALR, CARD11, CBL, CBLB, CBLC, CCND1, CCND2, CCNE1, CD19, CD274, CD74, CDH1, CDK12, CDK4, CDK6, CDK8, CDK9, CDKN1A, CDKN1C, CDKN2A, CDKN2B, CEBPA, CHD1, CHD3, CHD8, CHEK1, CHEK2, COG5, CRADD, CREBBP, CRLF2, CRX, CSF1R, CSF3R, CTCF, CTNNA1, CTNNB1, CUX1, DAXX, DDR2, DDX41, DEPDC5, DICER1, DIS3L2, DNAJB1, DNMT3A, DOCK7, DPYD, EBF1, EED, EGFR, EGLN1, EIF3E, EML4, ENPP3, EP300, EPAS1, EPCAM, EPHA3, EPHA5, EPHB2, EPHB6, EPO, EPOR, ERBB2, ERBB3, ERBB4, ERCC2, ERG, ESR1, ESR2, ETV6, EZH2, FAM175A, FAM19A2, FANCA, FANCB, FANCC, FANCD2, FANCE, FANCF, FANCG, FANCI, FANCL, FANCM, FBXW7, FGFR1, FGFR2, FGFR3, FGFR4, FH, FKBPIA, FLT1, FLT3, FLT4, FOXA1, FOXR2, FUBP1, GAB2, GALNT12, GATA1, GATA2, GATA3. GEN1, GLI1, GLI3, GLTSCR1, GLTSCR2, GNA11, GNAQ, GNAS, GPC3, GREM1, GRIN2A, GRM3, GYNPTH, H3-3A/H3F3A, H3-3B/H3F3B, H3C2/HISTIH3B, HDAC4, HDAC9, HIF1A, HNF1A, HNRNPU, HOOK3, HRAS, HSPH1, ID3, IDH, IDH1, IDH2, IGF1R, IKZF1, IL7R, JAK1, JAK2, JAK3, KCNJ8, KDM2B, KDM6A, KDR, KIF5B, KIT, KLF4, KMT2A, KMT2C, KMT2D, KRAS, KTN1, LYST, MAP2K1 (MEK1), MAP2K2 (MEK2), MAP2K4, MAP7, MAPK1, MAX, MC1R, MCL1, MDM2, MDM4, MED12, MEGF6, MEN1, MET, microsatellite instability, MIOS, MITF, MLH1, MLH3, MN1, MPL, MRE11A, MSH2, MSH6, MTAP, MTOR, MUTYH, MYB, MYC, MYCL, MYCL1, MYCN, MYD88, MYOD1, NAB2, NAT2, NBN, NF1, NF2, NKX2-1, NOTCH1, NOTCH2, NOTCH3, NPM1, NPRL2, NPRL3, NR4A3, NRAS, NRP1, NSD1, NT5C2, NTHL1, NTRK1, NTRK2, NTRK3, NUDT15, OFD1, PAK1, PALB2, PAX5, PBRM1, PDCDILG2, PDGFRA, PDGFRB, PHF6, PHOX2B, PIGA, PIK3CA, PIK3CB, PIK3R1, PLCG2, PLK1, PLK2, PLK3, PLK4, PML, PMS2, POLD1, POLE, PPM1D, PPP1CB, PRKAR1A, PRPF40B, PRPS1, PTCH1, PTEN, PTPN11, PTPRD, QKI, RAC1, RAD21, RAD51B, RAD51C, RAD51D, RAF1, RARA, RASA1, RB1, RECQL, RELA, RET, RHEB, RICTOR, RINT1, RIT1, ROR1, ROS1, RPL10, RPL31, RPS15, RPS20, RPTOR, RRM1, RRM2, RSPO2, RSPO3, RUNX1, SAMD9L, SDHA, SDHB, SDHC, SDHD, SETBP1, SETD2, SF1, SF3B1, SH2B3, SHH, SIGLEC10, SLC25A13, SLX4, SMAD2, SMAD3, SMAD4, SMARCA4, SMARCB1, SMC1A, SMC3, SMO, SNAPC3, SPOP, SPRY4, SRC, SRP72, SRSF2, STAG2, STAT5B, STAT6, STK11, SUFU, SUZ12, TACC3, TACSTD2, TCF3, TERC, TERT, TET1, TET2, TET3, TFE3, TFG, TGFBR2, THOR, THORplex, TLX1, TMB, TMPRSS2, total mutation burden. TP53, TP73, TRAF7, TRRAP, TSC1, TSC2, TTYH1, U2AFR, U2AF2, UBR5, USP7, VHL, WRN, WT1, XRCC2, YAP1, ZBTB16, ZFTA/C11orf95, and ZRSR2).

In some embodiments, the at least one cell parameter comprises screening for disease causing mutations in genes shown in Table 1 for cardiac cells; Table 2 for beta cells; Table 3 for T cells and Table 4 for neuronal cells.

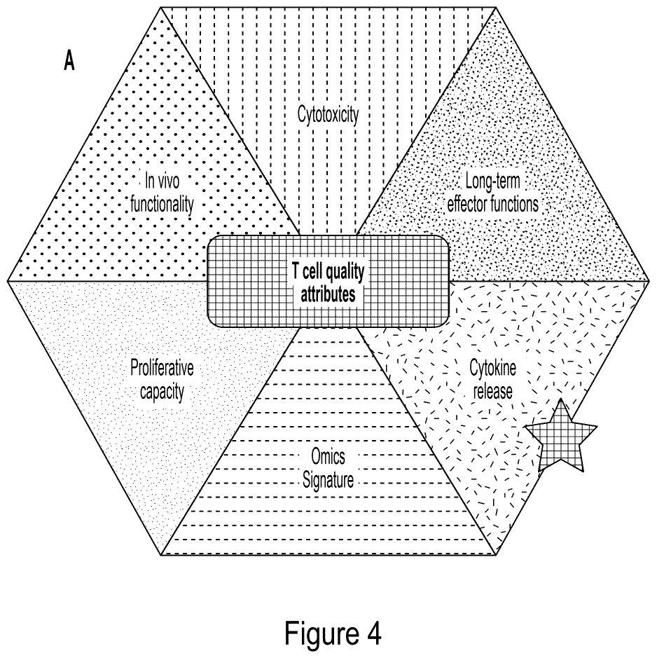

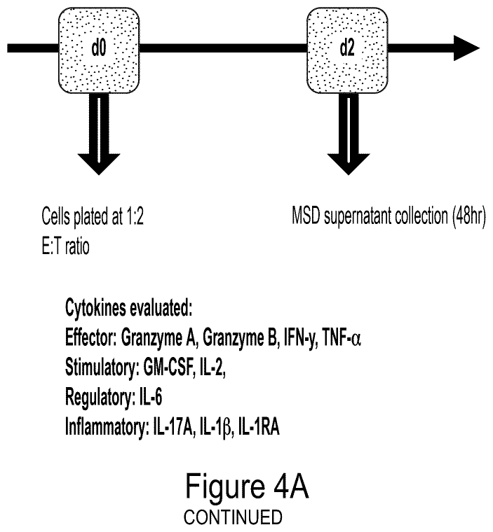

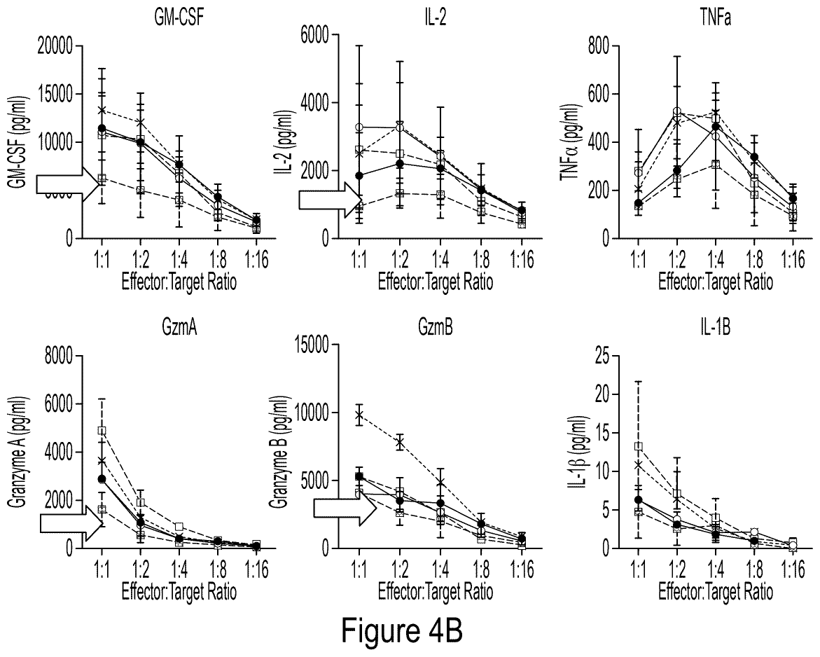

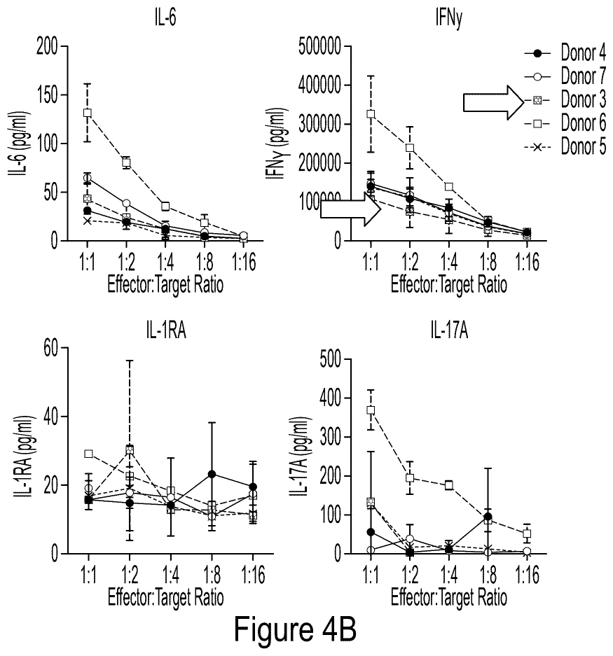

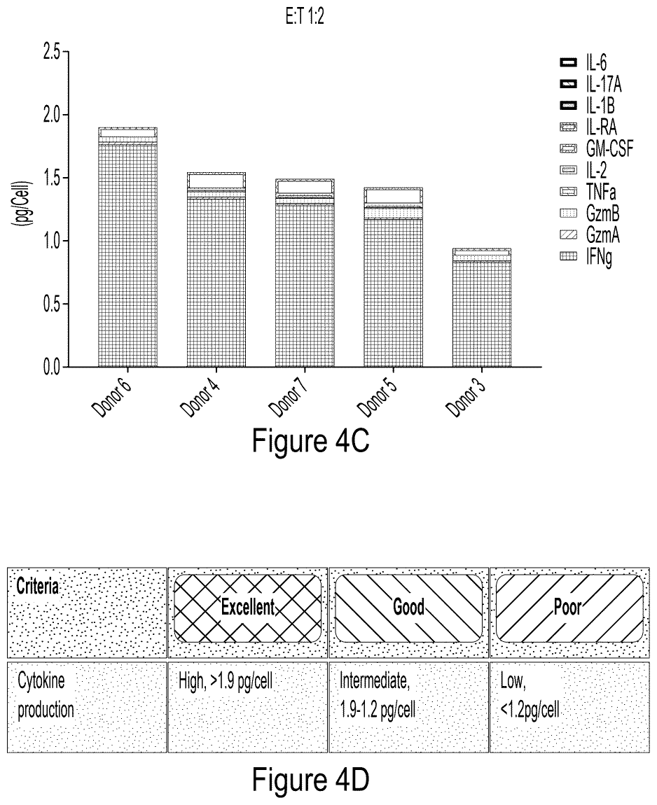

TABLE 1
Screening for disease causing mutations in genes
- genes of interest (GOI) for cardiac cells

| Gene | Disease |
|---|---|
| AAAS | Arrhythmia |
| AARS2 | Cardiomyopathy |
| ABCC6 | Cardiomyopathy |
| ABCC8 | Cardiomyopathy |
| ABCC9 | Cardiomyopathy |
| ACAD8 | Cardiomyopathy |
| ACAD9 | Cardiomyopathy |
| ACADL | Cardiomyopathy |
| ACADS | Cardiomyopathy |
| ACADVL | Cardiomyopathy |
| ACAT1 | Cardiomyopathy |
| ACE | Cardiomyopathy |
| ACN9 | Arrhythmia |
| ACTA1 | Cardiomyopathy |
| ACTC1 | Cardiomyopathy |
| ACTN2 | Cardiomyopathy |
| ADCY5 | Cardiomyopathy |
| ADH1B | Cardiomyopathy |
| ADH1C | Cardiomyopathy |
| ADLD | Arrhythmia |
| ADORA1 | |
| ADORA3 | |
| ADRA2C | |
| ADRB1 | Cardiomyopathy |
| ADRB2 | |
| ADRBK1 | |
| AGA | Cardiomyopathy |
| AGK | Cardiomyopathy |
| AGL | Cardiomyopathy |
| AGPAT2 | Cardiomyopathy |
| AGRN | Arrhythmia |
| AGT | |
| AGTR1 | Cardiomyopathy |
| AGTR2 | Cardiomyopathy |
| AIFM1 | Cardiomyopathy |
| AIP | Cardiomyopathy |
| AKAP10 | Arrhythmia |
| AKAP9 | Arrhythmia |
| ALG1 | Cardiomyopathy |
| ALG10 | Arrhythmia |
| ALG10B | Arrhythmia |
| ALG12 | Cardiomyopathy |
| ALMS1 | Cardiomyopathy |
| ALPK3 | Cardiomyopathy |
| AMER1 | Cardiomyopathy |
| ANK1 | Cardiomyopathy |
| ANK2 | Arrhythmia |
| ANK3 | Arrhythmia |
| ANKRD1 | Cardiomyopathy |
| ANKS6 | Cardiomyopathy |
| ANO5 | Cardiomyopathy |
| ANTXR1 | Cardiomyopathy |
| APOA1 | Cardiomyopathy |
| APOPT1 | Cardiomyopathy |
| ARNTL | Arrhythmia |
| ARSB | Cardiomyopathy |
| ARVD3 | Cardiomyopathy |
| ARVD4 | Cardiomyopathy |
| ASD1 | |
| ASXL2 | Arrhythmia |
| ATAD3A | Cardiomyopathy |
| ATP5E | Cardiomyopathy |
| ATP5F1D | Cardiomyopathy |
| ATP5MD | Cardiomyopathy |
| ATP6V1A | Cardiomyopathy |
| ATP7B | Cardiomyopathy |
| ATPAF2 | Cardiomyopathy |
| ATXN3 | Arrhythmia |
| AUH | Cardiomyopathy |
| B2M | Cardiomyopathy |
| BAG3 | Cardiomyopathy |
| BANF1 | Arrhythmia |
| BBC3 | Cardiomyopathy |
| BBS10 | Cardiomyopathy |
| BBS2 | Cardiomyopathy |
| BCS1L | Cardiomyopathy |
| BLM | |
| BMP10 | Cardiomyopathy |
| BMP2 | Cardiomyopathy |
| BOLA3 | Cardiomyopathy |
| BRAF | Cardiomyopathy |
| BRCC3 | Cardiomyopathy |
| BSCL2 | Cardiomyopathy |
| BVES | Cardiomyopathy |
| C10orf2 | |
| C1QBP | Cardiomyopathy |
| CACNA1C | Cardiomyopathy |
| CACNA1D | Arrhythmia |
| CACNA1S | Arrhythmia |
| CACNA2D1 | Arrhythmia |
| CACNB2 | Arrhythmia |
| CALM1 | Cardiomyopathy |
| CALM2 | Arrhythmia |
| CALM3 | Arrhythmia |
| CALR3 | Cardiomyopathy |
| CAMK2A | |
| CAMK2D | Cardiomyopathy |
| CAPN3 | Cardiomyopathy |
| CASP1 | Arrhythmia |
| CASQ2 | Arrhythmia |
| CASZ1 | Cardiomyopathy |
| CAV1 | Cardiomyopathy |
| CAV3 | Cardiomyopathy |
| CAVIN1 | Arrhythmia |
| CAVIN4 | Cardiomyopathy |
| CBL | Cardiomyopathy |
| CD36 | Cardiomyopathy |
| CD3D | Arrhythmia |
| CDH1 | Cardiomyopathy |
| CDH2 | Cardiomyopathy |
| CDK9 | Cardiomyopathy |
| CDKN1C | Cardiomyopathy |
| CELF1 | Cardiomyopathy |
| CENPE | Cardiomyopathy |
| CFH | Cardiomyopathy |
| CFHR1 | Cardiomyopathy |
| CFHR3 | Cardiomyopathy |
| CHKB | Cardiomyopathy |
| CHRM2 | Cardiomyopathy |
| CITED2 | |
| CLCNKB | Arrhythmia |
| CLDN16 | |
| CLN3 | Cardiomyopathy |
| CLPB | Cardiomyopathy |
| CMD1A | Arrhythmia |
| CMD1B | Cardiomyopathy |
| CMD1EE | Cardiomyopathy |
| CMD1H | Cardiomyopathy |
| CMD1K | Cardiomyopathy |
| CMD1NN | Arrhythmia |
| CMD1Q | Cardiomyopathy |
| CMYA1 | Cardiomyopathy |
| CMYA3 | Cardiomyopathy |
| CMYA5 | Cardiomyopathy |
| CNBP | Cardiomyopathy |
| CNOT3 | Cardiomyopathy |
| COA5 | Cardiomyopathy |
| COA6 | Cardiomyopathy |
| COL15A1 | Cardiomyopathy |
| COL4A1 | Cardiomyopathy |
| COL6A1 | Cardiomyopathy |
| COL6A2 | Cardiomyopathy |
| COL6A3 | Cardiomyopathy |
| COL7A1 | Cardiomyopathy |
| COQ2 | Cardiomyopathy |
| COQ4 | Cardiomyopathy |
| CORIN | Cardiomyopathy |
| COX10 | Cardiomyopathy |
| COX14 | Cardiomyopathy |
| COX15 | Cardiomyopathy |
| COX20 | Cardiomyopathy |
| COX6A2 | Cardiomyopathy |
| COX6A2 | Cardiomyopathy |
| COX6B1 | Cardiomyopathy |
| COX8A | Cardiomyopathy |
| CPOX | Arrhythmia |
| CPT1A | |
| CPT2 | Cardiomyopathy |
| CR1 | Cardiomyopathy |
| CR2 | Cardiomyopathy |
| CREB1 | Cardiomyopathy |
| CREBBP | Arrhythmia |
| CRPPA | Cardiomyopathy |
| CRYAB | Cardiomyopathy |
| CSF2RA | Arrhythmia |
| CSF3R | |
| CSRP3 | Cardiomyopathy |
| CTF1 | |
| CTLA4 | |
| CTNNA1 | Cardiomyopathy |
| CTNNA3 | Cardiomyopathy |
| CTSD | Cardiomyopathy |
| CTSL | Cardiomyopathy |
| CUL3 | Cardiomyopathy |
| CXADR | Cardiomyopathy |
| CXCL12 | Cardiomyopathy |
| CYB561 | Arrhythmia |
| CYBB | Cardiomyopathy |
| CYP11B1 | Cardiomyopathy |
| CYP11B2 | Cardiomyopathy |
| CYP1A1 | Cardiomyopathy |
| CYP2B6 | Cardiomyopathy |
| CYP2C18 | Cardiomyopathy |
| CYP2C19 | Cardiomyopathy |
| CYP2C8 | Cardiomyopathy |
| CYP2C9 | Cardiomyopathy |
| CYP2D6 | Cardiomyopathy |
| CYP4B1 | Cardiomyopathy |
| CYR61 | Cardiomyopathy |
| D2HGA2 | Cardiomyopathy |
| D2HGDH | Cardiomyopathy |
| DAAM1 | Cardiomyopathy |
| DCAF8 | Cardiomyopathy |
| DES | Cardiomyopathy |
| DICER1 | Cardiomyopathy |
| DLD | Cardiomyopathy |
| DMD | Cardiomyopathy |
| DMPK | Cardiomyopathy |
| DNAJA3 | Cardiomyopathy |
| DNAJB6 | Cardiomyopathy |
| DNAJC19 | Cardiomyopathy |
| DOK7 | |
| DOLK | Cardiomyopathy |
| DPM3 | Cardiomyopathy |
| DRD4 | Arrhythmia |
| DSC2 | Cardiomyopathy |
| DSC3 | |
| DSG1 | Cardiomyopathy |
| DSG2 | Cardiomyopathy |
| DSP | Cardiomyopathy |
| DST | Arrhythmia |
| DTNA | Cardiomyopathy |
| DUSP4 | Cardiomyopathy |
| DYSF | Cardiomyopathy |
| ECE1 | Arrhythmia |
| ECHS1 | Cardiomyopathy |
| ECSCR | Cardiomyopathy |
| EDN1 | Cardiomyopathy |
| EDNRB | Cardiomyopathy |
| EEF1A2 | Cardiomyopathy |
| EGFR | |
| ELAC2 | Cardiomyopathy |
| ELN | |
| ELP1 | Arrhythmia |
| EMD | Cardiomyopathy |
| ENPP1 | Coronary artery disease |
| EPG5 | Cardiomyopathy |
| EPHX2 | |
| ERBB2 | Cardiomyopathy |
| ERBB3 | Cardiomyopathy |
| ERCC6 | |
| ERCC8 | |
| ESRRG | |
| ETFA | Cardiomyopathy |
| ETFB | Cardiomyopathy |
| ETFDH | Cardiomyopathy |
| EYA2 | Arrhythmia |
| EYA3 | Arrhythmia |
| EYA4 | Cardiomyopathy |
| F5 | |
| FAH | Cardiomyopathy |
| FASTKD2 | Cardiomyopathy |
| FBN1 | Cardiomyopathy |
| FBP1D | Arrhythmia |
| FBXL4 | Cardiomyopathy |
| FBXO32 | Cardiomyopathy |
| FCGR2B | Cardiomyopathy |
| FER1L3 | Cardiomyopathy |
| FGA | Cardiomyopathy |
| FHL1 | Cardiomyopathy |
| FHL1TV1 | |
| FHL2 | |
| FHOD3 | Arrhythmia |
| FIG. 4 | Cardiomyopathy |
| FKBP14 | Cardiomyopathy |
| FKBP1A | Cardiomyopathy |
| FKBP1B | Arrhythmia |
| FKRP | Cardiomyopathy |
| FKTN | Cardiomyopathy |
| FLAD1 | Cardiomyopathy |
| FLNC | Cardiomyopathy |
| FLT1 | Cardiomyopathy |
| FMO3 | Arrhythmia |
| FMR1 | |
| FNDC1 | Arrhythmia |
| FNIP1 | Cardiomyopathy |
| FOXC2 | |
| FOXD4 | Cardiomyopathy |
| FOXRED1 | Cardiomyopathy |
| FTO | Cardiomyopathy |
| FXN | Cardiomyopathy |
| FXR1 | Arrhythmia |
| GAA | Cardiomyopathy |
| GABRA2 | Cardiomyopathy |
| GALR1 | Arrhythmia |
| GATA4 | Cardiomyopathy |
| GATA5 | Cardiomyopathy |
| GATA6 | Congenital defects |
| GATAD1 | Cardiomyopathy |
| GATB | Cardiomyopathy |
| GATC | Cardiomyopathy |
| GBE1 | Cardiomyopathy |
| GCDH | Cardiomyopathy |
| GCOM1 | |
| GDNF | Arrhythmia |
| GFM1 | Cardiomyopathy |
| GFPT1 | Cardiomyopathy |
| GJA1 | Arrhythmia |
| GJA5 | Cardiomyopathy |
| GJB2 | Cardiomyopathy |
| GLA | Cardiomyopathy |
| GLB1 | Cardiomyopathy |
| GLI3 | Arrhythmia |
| GMPPB | Cardiomyopathy |
| GNAI2 | Arrhythmia |
| GNAO1 | Arrhythmia |
| GNAQ | Cardiomyopathy |
| GNAS | |
| GNB5 | Arrhythmia |
| GNPTAB | Cardiomyopathy |
| GPC3 | Cardiomyopathy |
| GPD1L | Arrhythmia |
| GPX4 | Arrhythmia |
| GRK2 | Cardiomyopathy |
| GRK5 | Cardiomyopathy |
| GSK3B | Cardiomyopathy |
| GSN | Cardiomyopathy |
| GTPBP3 | Cardiomyopathy |
| GUCY2D | Cardiomyopathy |
| GUSB | Cardiomyopathy |
| GYG1 | Arrhythmia |
| GYS1 | Cardiomyopathy |
| H19 | Cardiomyopathy |
| HADH | |
| HADHA | Cardiomyopathy |
| HADHB | Cardiomyopathy |
| HADHSC | Cardiomyopathy |
| HAMP | Cardiomyopathy |
| HAND1 | Cardiomyopathy |
| HAND2 | Cardiomyopathy |
| HCCS | Cardiomyopathy |
| HCN4 | Cardiomyopathy |
| HDAC1 | Cardiomyopathy |
| HDAC2 | Cardiomyopathy |
| HDAC3 | Cardiomyopathy |
| HDAC4 | |
| HESX1 | Cardiomyopathy |
| HEY2 | Cardiomyopathy |
| HFE | Cardiomyopathy |
| HFE2 | Cardiomyopathy |
| HIF1A | |
| HJV | Cardiomyopathy |
| HK1 | |
| HLA-DRB1 | Cardiomyopathy |
| HMBS | Arrhythmia |
| HPS1 | Cardiomyopathy |
| HRAS | Cardiomyopathy |
| HRC | |
| HSD17B10 | Cardiomyopathy |
| HSPB7 | |
| HTR2A | Cardiomyopathy |
| ICMT | Arrhythmia |
| ICR1 | Cardiomyopathy |
| IDH2 | Cardiomyopathy |
| IDS | Cardiomyopathy |
| IDUA | Cardiomyopathy |
| IFNG | Cardiomyopathy |
| IGHMBP2 | Cardiomyopathy |
| IKBKAP | |
| IL10 | |
| IL17A | Cardiomyopathy |
| IL6ST | Cardiomyopathy |
| ILK | Cardiomyopathy |
| INS-IGF2 | |
| IRX5 | |
| ISCU | Cardiomyopathy |
| ISL1 | |
| ITGB1BP2 | Cardiomyopathy |
| ITPA | Cardiomyopathy |
| JPH2 | Cardiomyopathy |
| JUP | Cardiomyopathy |
| KANK4 | Cardiomyopathy |
| KARS1 | Cardiomyopathy |
| KAT6B | Cardiomyopathy |
| KCNA4 | Arrhythmia |
| KCNA5 | Arrhythmia |
| KCNC1 | Arrhythmia |
| KCND3 | Arrhythmia |
| KCNE1 | Arrhythmia |
| KCNE2 | Cardiomyopathy |
| KCNE3 | Arrhythmia |
| KCNE5 | Arrhythmia |
| KCNH1 | Cardiomyopathy |
| KCNH2 | Arrhythmia |
| KCNH2TV3 | |
| KCNIP2 | Arrhythmia |
| KCNJ1 | |
| KCNJ11 | Cardiomyopathy |
| KCNJ18 | Arrhythmia |
| KCNJ2 | Arrhythmia |
| KCNJ5 | Arrhythmia |
| KCNJ8 | Arrhythmia |
| KCNK17 | |
| KCNK3 | |
| KCNQ1 | Arrhythmia |
| KCNQ1OT1 | Cardiomyopathy |
| KCNQ2 | Arrhythmia |
| KCNQ3 | Arrhythmia |
| KDELR1 | Cardiomyopathy |
| KIF1B | Arrhythmia |
| KLF1 | Cardiomyopathy |
| KLF10 | |
| KLF15 | |
| KLH24 | Cardiomyopathy |
| KLHL3 | |
| KRAS | Cardiomyopathy |
| KYNU | Arrhythmia |
| LAMA2 | Cardiomyopathy |
| LAMA4 | Cardiomyopathy |
| LAMP2 | Cardiomyopathy |
| LARGE1 | Cardiomyopathy |
| LDB3 | Cardiomyopathy |
| LDB3Z4 | |
| LEMD2 | Cardiomyopathy |
| LIAS | Cardiomyopathy |
| LIMS2 | Cardiomyopathy |
| LMNA | Cardiomyopathy |
| LMNA2 | |
| LMNB1 | Cardiomyopathy |
| LMOD2 | Cardiomyopathy |
| LOX | Arrhythmia |
| LPA | |
| LPAR6 | Cardiomyopathy |
| LPL | Cardiomyopathy |
| LRPPRC | Cardiomyopathy |
| LRRC10 | Cardiomyopathy |
| LTBP4 | Cardiomyopathy |
| LYZ | Cardiomyopathy |
| LZTR1 | Cardiomyopathy |
| MACF1 | Arrhythmia |
| MADD | Cardiomyopathy |
| MAP2K1 | Cardiomyopathy |
| MAP2K2 | Cardiomyopathy |
| MAP3KB | Noonan |
| MAPK14 | Cardiomyopathy |
| MAX | Arrhythmia |
| MBNL1 | Arrhythmia |
| MC5DN3 | Cardiomyopathy |
| MECP2 | Arrhythmia |
| MEF2A | Cardiomyopathy |
| MEF2C | Cardiomyopathy |
| MFN2 | Cardiomyopathy |
| MGME1 | Cardiomyopathy |
| MHRT | Cardiomyopathy |
| MIB1 | Cardiomyopathy |
| MICOS13 | Cardiomyopathy |
| MIPEP | Cardiomyopathy |
| MIR133A1 | Cardiomyopathy |
| MIR133A2 | Cardiomyopathy |
| MIR196A2 | |
| MIR499A | |
| MKKS | Cardiomyopathy |
| MKL1 | |
| MLH3 | Cardiomyopathy |
| MLYCD | Cardiomyopathy |
| MMP1 | Cardiomyopathy |
| MMUT | Cardiomyopathy |
| MMVP1 | Cardiomyopathy |
| MPZ | Cardiomyopathy |
| MRAS | Cardiomyopathy |
| MRPL3 | Cardiomyopathy |
| MRPL44 | Cardiomyopathy |
| MRPS14 | Cardiomyopathy |
| MRPS22 | Cardiomyopathy |
| MRTFA | Cardiomyopathy |
| MSH3 | Cardiomyopathy |
| MSH6 | Cardiomyopathy |
| MT-TA | Cardiomyopathy |
| MT-TC | Cardiomyopathy |
| MT-TD | Cardiomyopathy |
| MT-TE | Cardiomyopathy |
| MT-TF | Cardiomyopathy |
| MT-TG | Cardiomyopathy |
| MT-TH | Cardiomyopathy |
| MT-TI | Cardiomyopathy |
| MT-TK | Cardiomyopathy |
| MT-TL1 | Cardiomyopathy |
| MT-TL2 | Cardiomyopathy |
| MT-TM | Cardiomyopathy |
| MT-TN | Cardiomyopathy |
| MT-TP | Cardiomyopathy |
| MT-TQ | Cardiomyopathy |
| MT-TR | Cardiomyopathy |
| MT-TS1 | Cardiomyopathy |
| MT-TS2 | Cardiomyopathy |
| MT-TT | Cardiomyopathy |
| MT-TV | Cardiomyopathy |
| MT-TW | Cardiomyopathy |
| MT-TY | Cardiomyopathy |
| MTATP6 | Cardiomyopathy |
| MTATP8 | Cardiomyopathy |
| MTCO1 | Cardiomyopathy |
| MTCO2 | Cardiomyopathy |
| MTCO3 | Cardiomyopathy |
| MTCP1 | Cardiomyopathy |
| MTCYB | Cardiomyopathy |
| MTND1 | Cardiomyopathy |
| MTND2 | Cardiomyopathy |
| MTND3 | Cardiomyopathy |
| MTND4 | Arrhythmia |
| MTND4L | Cardiomyopathy |
| MTND5 | Cardiomyopathy |
| MTND6 | Cardiomyopathy |
| MTO1 | Cardiomyopathy |
| MTRNR1 | Cardiomyopathy |
| MTRNR2 | Cardiomyopathy |
| MURC | |
| MUT | |
| MYBPC1 | Cardiomyopathy |
| MYBPC3 | Cardiomyopathy |
| MYBPHL | Cardiomyopathy |
| MYF6 | Cardiomyopathy |
| MYH10 | |
| MYH6 | Cardiomyopathy |
| MYH7 | Cardiomyopathy |
| MYH7B | |
| MYL2 | Cardiomyopathy |
| MYL3 | Cardiomyopathy |
| MYL4 | Arrhythmia |
| MYLCD | Cardiomyopathy |
| MYLK2 | Cardiomyopathy |
| MYO18B | Cardiomyopathy |
| MYO6 | Cardiomyopathy |
| MYOCD | Cardiomyopathy |
| MYOF | |
| MYOM1 | |
| MYOT | Cardiomyopathy |
| MYOZ2 | Cardiomyopathy |
| MYPN | Cardiomyopathy |
| MYRF | Congenital defects |
| MYSM1 | Cardiomyopathy |
| MYZAP | Cardiomyopathy |
| NAA10 | Arrhythmia |
| NAXD | Cardiomyopathy |
| NBAS | Cardiomyopathy |
| NCOA6 | |
| NDUFA1 | |
| NDUFA10 | Cardiomyopathy |
| NDUFA11 | Cardiomyopathy |
| NDUFA12 | |
| NDUFA2 | |
| NDUFA9 | |
| NDUFAF1 | Cardiomyopathy |
| NDUFAF2 | Cardiomyopathy |
| NDUFAF3 | |
| NDUFAF4 | Cardiomyopathy |
| NDUFAF5 | |
| NDUFAF6 | |
| NDUFB11 | Cardiomyopathy |
| NDUFB11 | Cardiomyopathy |
| NDUFB3 | |
| NDUFB9 | |
| NDUFS1 | |
| NDUFS2 | Cardiomyopathy |
| NDUFS3 | |
| NDUFS4 | Cardiomyopathy |
| NDUFS6 | |
| NDUFS7 | Arrhythmia |
| NDUFS8 | Cardiomyopathy |
| NDUFV1 | |
| NDUFV2 | Cardiomyopathy |
| NEBL | Cardiomyopathy |
| NEK8 | Cardiomyopathy |
| NEU1 | Cardiomyopathy |
| NEXN | Cardiomyopathy |
| NF1 | Cardiomyopathy |
| NFE2L2 | Cardiomyopathy |
| NFKB1 | |
| NHLH1 | Arrhythmia |
| NKX2-5 | Cardiomyopathy |
| NNT | Cardiomyopathy |
| NONO | Cardiomyopathy |
| NOS1 | Cardiomyopathy |
| NOS1AP | Cardiomyopathy |
| NOS3 | Cardiomyopathy |
| NOTCH1 | Cardiomyopathy |
| NPPA | Cardiomyopathy |
| NR3C2 | Cardiomyopathy |
| NRAP | Cardiomyopathy |
| NRAS | Cardiomyopathy |
| NRG1 | Cardiomyopathy |
| NSD1 | |
| NTRK1 | Arrhythmia |
| NUBPL | |
| NUP107 | Cardiomyopathy |
| NUP155 | Cardiomyopathy |
| NUTMB2-AS1 | Cardiomyopathy |
| OBSCN | |
| OCLN | Cardiomyopathy |
| OPA1 | Cardiomyopathy |
| OPA3 | Cardiomyopathy |
| PAM16 | Cardiomyopathy |
| PARK2 | Cardiomyopathy |
| PARS2 | Cardiomyopathy |
| PCCA | Cardiomyopathy |
| PCCB | Cardiomyopathy |
| PDCD1 | Cardiomyopathy |
| PDGFRA | Cardiomyopathy |
| PDGFRB | Cardiomyopathy |
| PDLIM3 | Cardiomyopathy |
| PDLIM5 | Cardiomyopathy |
| PDPK1 | Cardiomyopathy |
| PER1 | Arrhythmia |
| PER2 | Arrhythmia |
| PET100 | Cardiomyopathy |
| PEX5 | Arrhythmia |
| PEX7 | Cardiomyopathy |
| PFKM | Cardiomyopathy |
| PGM1 | Cardiomyopathy |
| PHYH | Cardiomyopathy |
| PHYHIP | Arrhythmia |
| PIGT | Cardiomyopathy |
| PIGU | Arrhythmia |
| PIK3CA | Arrhythmia |
| PINK1 | Cardiomyopathy |
| PKD2 | |
| PKP2 | Cardiomyopathy |
| PLEC | Cardiomyopathy |
| PLEC1 | Cardiomyopathy |
| PLEKHM2 | Arrhythmia |
| PLN | Cardiomyopathy |
| PMM2 | Cardiomyopathy |
| PNPLA2 | Cardiomyopathy |
| PNPLA8 | Cardiomyopathy |
| POLG | Cardiomyopathy |
| POMT1 | Cardiomyopathy |
| POU5F1 | Cardiomyopathy |
| PPA2 | Cardiomyopathy |
| PPARD | Cardiomyopathy |
| PPARG | Cardiomyopathy |
| PPARGC1A | Cardiomyopathy |
| PPARGC1B | Cardiomyopathy |
| PPCS | Cardiomyopathy |
| PPCS | Cardiomyopathy |
| PPIF | Cardiomyopathy |
| PPOX | Arrhythmia |
| PPP1CA | Cardiomyopathy |
| PPP1CB | Congenital defects |
| PPP1R13L | Cardiomyopathy |
| PPP3CA | Cardiomyopathy |
| PRDM16 | Cardiomyopathy |
| PRF1 | Cardiomyopathy |
| PRKAG2 | Cardiomyopathy |
| PRKCA | Cardiomyopathy |
| PRKCB | Arrhythmia |
| PROC | |
| PSEN1 | Cardiomyopathy |
| PSEN2 | Cardiomyopathy |
| PSMB8 | |
| PTEN | |
| PTPN11 | Cardiomyopathy |
| PTRF | |
| QRSL1 | Cardiomyopathy |
| RAB3GAP2 | Cardiomyopathy |
| RAF1 | Cardiomyopathy |
| RANGRF | Arrhythmia |
| RAPSN | |
| RASA2 | Cardiomyopathy |
| RBCK1 | Cardiomyopathy |
| RBM10 | Cardiomyopathy |
| RBM20 | Cardiomyopathy |
| RBMX | Cardiomyopathy |
| RCBTB1 | |
| RECQL2 | Cardiomyopathy |
| RECQL3 | Cardiomyopathy |
| REST | Cardiomyopathy |
| RET | Arrhythmia |
| RETN | |
| RICTOR | Cardiomyopathy |
| RIT1 | Cardiomyopathy |
| RMND1 | Cardiomyopathy |
| RNF207 | |
| RNF31 | Cardiomyopathy |
| RNU4ATAC | Arrhythmia |
| RPS6KA3 | Cardiomyopathy |
| RPSA | Cardiomyopathy |
| RRAS | Cardiomyopathy |
| RRAS2 | Cardiomyopathy |
| RYR1 | Cardiomyopathy |
| RYR2 | Cardiomyopathy |
| SALL4 | Congenital defects |
| SANDD | Arrhythmia |
| SARDH | Cardiomyopathy |
| SCN10A | Arrhythmia |
| SCN1B | Arrhythmia |
| SCN1BB | |
| SCN2B | Arrhythmia |
| SCN3B | Arrhythmia |
| SCN4A | Arrhythmia |
| SCN4B | Arrhythmia |
| SCN5A | Cardiomyopathy |
| SCN8A | Cardiomyopathy |
| SCN9A | Arrhythmia |
| SCNN1B | Arrhythmia |
| SCO1 | Cardiomyopathy |
| SCO2 | Cardiomyopathy |
| SCRIB | Cardiomyopathy |
| SDHA | Cardiomyopathy |
| SDHAF1 | Cardiomyopathy |
| SDHB | Arrhythmia |
| SDHC | Arrhythmia |
| SDHD | Cardiomyopathy |
| SELENON | Cardiomyopathy |
| SELENOP | Cardiomyopathy |
| SEMA3A | Arrhythmia |
| SEPN1 | |
| SEPP1 | |
| SGCA | Cardiomyopathy |
| SGCB | Cardiomyopathy |
| SGCD | Cardiomyopathy |
| SGCDP | |
| SGCG | Cardiomyopathy |
| SGOL1 | Arrhythmia |
| SHOC2 | Cardiomyopathy |
| SHOX | Arrhythmia |
| SIRT7 | Cardiomyopathy |
| SKI | Cardiomyopathy |
| SLC12A3 | Arrhythmia |
| SLC19A2 | Cardiomyopathy |
| SLC22A5 | Cardiomyopathy |
| SLC25A20 | Cardiomyopathy |
| SLC25A3 | Cardiomyopathy |
| SLC25A4 | Cardiomyopathy |
| SLC30A10 | Cardiomyopathy |
| SLC36A2 | |
| SLC40A1 | Cardiomyopathy |
| SLC6A19 | Arrhythmia |
| SLC6A2 | Arrhythmia |
| SLC6A20 | Arrhythmia |
| SLPI | |
| SMAD4 | Cardiomyopathy |
| SMARCA4 | Cardiomyopathy |
| SMC1A | Cardiomyopathy |
| SMC1A | Cardiomyopathy |
| SMCHD1 | Cardiomyopathy |
| SMPD4 | Cardiomyopathy |
| SNAP25 | Arrhythmia |
| SNTA1 | Arrhythmia |
| SOCS1 | Cardiomyopathy |
| SOD2 | Cardiomyopathy |
| SOS1 | Cardiomyopathy |
| SOS2 | Cardiomyopathy |
| SP4 | Arrhythmia |
| SPEG | Cardiomyopathy |
| SPP1 | Cardiomyopathy |
| SPRED1 | Cardiomyopathy |
| SPTLC2 | Cardiomyopathy |
| SREBF1 | Cardiomyopathy |
| SRFBP1 | Cardiomyopathy |
| SRI | |
| SSTR5 | |
| STAG2 | Congenital defects |
| STRN | Cardiomyopathy |
| SUCLA2 | Cardiomyopathy |
| SUCLG1 | Cardiomyopathy |
| SUFU | Cardiomyopathy |
| SURF1 | Cardiomyopathy |
| SYNE1 | Cardiomyopathy |
| SYNE2 | Cardiomyopathy |
| SYNM | |
| TAB2 | Congenital defects |
| TAC4 | Arrhythmia |
| TACO1 | Cardiomyopathy |
| TANGO2 | Cardiomyopathy |
| TAPT1 | Cardiomyopathy |
| TAS2R16 | |
| TAX1BP3 | Cardiomyopathy |
| TAZ | Cardiomyopathy |
| TBX20 | Cardiomyopathy |
| TBX3 | Arrhythmia |
| TBX5 | Arrhythmia |
| TCAP | Cardiomyopathy |
| TCF21 | Cardiomyopathy |
| TDP2 | Arrhythmia |
| TECRL | Arrhythmia |
| TERC | Cardiomyopathy |
| TERT | Cardiomyopathy |
| TFAM | Cardiomyopathy |
| TFB1M | Cardiomyopathy |
| TFR2 | Cardiomyopathy |
| TGFB3 | Cardiomyopathy |
| THRA | Cardiomyopathy |
| THRB | Arrhythmia |
| TIA1 | Cardiomyopathy |
| TICAM1 | Cardiomyopathy |
| TIRAP | |
| TKFC | Cardiomyopathy |
| TLL1 | |
| TLR4 | |
| TLR9 | Cardiomyopathy |
| TMEM126A | Cardiomyopathy |
| TMEM126B | Cardiomyopathy |
| TMEM127 | Arrhythmia |
| TMEM43 | Cardiomyopathy |
| TMEM70 | Cardiomyopathy |
| TMEM87B | Cardiomyopathy |
| TMPO | Cardiomyopathy |
| TNF | Arrhythmia |
| TNFSF12 | Cardiomyopathy |
| TNNC1 | Cardiomyopathy |
| TNNI2 | Cardiomyopathy |
| TNNI3 | Cardiomyopathy |
| TNNI3K | Cardiomyopathy |
| TNNT2 | Cardiomyopathy |
| TNNT2I1 | |
| TNNT3 | Cardiomyopathy |
| TOP3A | Arrhythmia |
| TOR1AIP1 | Cardiomyopathy |
| TP53 | Cardiomyopathy |
| TP63 | Cardiomyopathy |
| TPM1 | Cardiomyopathy |
| TPM17 | |
| TPM3 | Cardiomyopathy |
| TRA2B | Cardiomyopathy |
| TRDN | Arrhythmia |
| TRIM32 | Cardiomyopathy |
| TRIM37 | Cardiomyopathy |
| TRIM54 | |
| TRIM55 | |
| TRIM63 | |
| TRMT5 | Cardiomyopathy |
| TRNT1 | Cardiomyopathy |
| TRPA1 | Arrhythmia |
| TRPC6 | Cardiomyopathy |
| TRPM4 | Familal heart block |
| TSC1 | Cardiomyopathy |
| TSC2 | Arrhythmia |
| TSFM | Cardiomyopathy |
| TSHR | Arrhythmia |
| TTN | Cardiomyopathy |
| TTNLTV | |
| TTNTVN2B | |
| TTPA | Cardiomyopathy |
| TTR | Cardiomyopathy |
| TWNK | Cardiomyopathy |
| TXNRD2 | Cardiomyopathy |
| UBR1 | Cardiomyopathy |
| UQCRFS1 | Cardiomyopathy |
| VARS2 | Cardiomyopathy |
| VAV3 | Arrhythmia |
| VCL | Cardiomyopathy |
| VCP | Cardiomyopathy |
| VEGFA | |
| VHL | Arrhythmia |
| VPS13A | |
| VPS13B | Cardiomyopathy |
| VPS33A | Cardiomyopathy |
| WARS2 | Cardiomyopathy |
| WFS1 | Cardiomyopathy |
| WRN | |
| WT1 | Cardiomyopathy |
| XIRP1 | Cardiomyopathy |
| XIRP2 | |
| XK | Cardiomyopathy |
| XLD | Cardiomyopathy |
| XPNPEP3 | Cardiomyopathy |
| XRCC4 | Cardiomyopathy |
| XYLT1 | Cardiomyopathy |
| XYLT2 | Cardiomyopathy |
| YARS2 | Cardiomyopathy |
| YME1L1 | Cardiomyopathy |
| YWHAG | Cardiomyopathy |
| YY1AP1 | Cardiomyopathy |
| ZIC3 | |
| ZMPSTE24 | Cardiomyopathy |

TABLE 2
Screening for disease causing mutations in genes
- genes of interest (GOI) for beta cells

| Gene | Disease |
|---|---|
| ABCC80 | Monogenic diabetes |
| AGPAT2 | Monogenic diabetes |
| AIRE | Monogenic diabetes |
| AKT2 | Monogenic diabetes |
| APPL1 | Monogenic diabetes |
| BSCL2 | Monogenic diabetes |
| CD55 | Monogenic diabetes |
| CEL | Monogenic diabetes |
| CIDEC | Monogenic diabetes |
| CISD2 | Monogenic diabetes |
| CNOT1 | Monogenic diabetes |
| COQ2 | Monogenic diabetes |
| COQ9 | Monogenic diabetes |
| CTLA4 | Monogenic diabetes |
| DCAF17 | Monogenic diabetes |
| DNAJC3 | Monogenic diabetes |
| DOCK8 | Monogenic diabetes |
| DUT | Monogenic diabetes |
| DYRK1B | Monogenic diabetes |
| EIF2AK3 | Monogenic diabetes |
| EIF2S3 | Monogenic diabetes |
| FOXP3 | Monogenic diabetes |
| GATA4 | Monogenic diabetes |
| GATA6 | Monogenic diabetes |
| GCK | Monogenic diabetes |
| GLIS3 | Monogenic diabetes |
| HNF1A | Monogenic diabetes |
| HNF1B | Monogenic diabetes |
| HNF4A | Monogenic diabetes |
| IER3IP1 | Monogenic diabetes |
| IL2RA | Monogenic diabetes |
| INS | Monogenic diabetes |
| INSR | Monogenic diabetes |
| ITCH | Monogenic diabetes |
| JAK1 | Monogenic diabetes |
| KCNJ11 | Monogenic diabetes |
| LIPE | Monogenic diabetes |
| LMNA | Monogenic diabetes |
| LPL | Monogenic diabetes |
| LRBA | Monogenic diabetes |
| MANF | Monogenic diabetes |
| MNX1 | Monogenic diabetes |
| MTTL1 | Monogenic diabetes |
| m.3243A > G | |
| NEUROD1 | Monogenic diabetes |
| NEUROG3 | Monogenic diabetes |
| NFKB1 | Monogenic diabetes |
| NKX2-2 | Monogenic diabetes |
| PAX6 | Monogenic diabetes |
| PCBD1 | Monogenic diabetes |
| PDX1 | Monogenic diabetes |
| PIK3R1 | Monogenic diabetes |
| PLIN1 | Monogenic diabetes |
| POLD1 | Monogenic diabetes |
| PPARG | Monogenic diabetes |
| PPP1R15B | Monogenic diabetes |
| PTF1A | Monogenic diabetes |
| RFX6 | Monogenic diabetes |
| SIRT1 | Monogenic diabetes |
| SLC2A2 | Monogenic diabetes |
| SLC19A2 | Monogenic diabetes |
| SLC29A3 | Monogenic diabetes |
| STAT1 | Monogenic diabetes |
| STAT3 | Monogenic diabetes |
| STAT5B | Monogenic diabetes |
| TNFAIP3 | Monogenic diabetes |
| TRMT10A | Monogenic diabetes |
| WFS1 | Monogenic diabetes |
| ZBTB20 | Monogenic diabetes |
| ZFP57 | Monogenic diabetes |
| ZMPSTE24 | Monogenic diabetes |
| MEN1 | Endocrine neoplasia |
| RET | Endocrine neoplasia |
| CDKN1B | Endocrine neoplasia |
| VHL | Endocrine neoplasia |
| NF1 | Endocrine neoplasia |
| GNAS | Endocrine neoplasia |
| PRKAR1A | Endocrine neoplasia |
| TSC1 | Endocrine neoplasia |
| TSC2 | Endocrine neoplasia |
| DAXX | Endocrine neoplasia |
| ATRX | Endocrine neoplasia |
| PIK3CA | Endocrine neoplasia |
| PTEN | Endocrine neoplasia |
| TP53 | Endocrine neoplasia |

TABLE 3
Screening for disease causing mutations in
genes - genes of interest (GOI) for T cells

| Gene | Disease |
|---|---|
| T cell deficiency | |
| SEVERE COMBINED | SCID DUE TO ADA DEFICIENCY, |
| IMMUNODEFICIENCY, AUTOSOMAL | DELAYED ONSET, INCLUDED |
| RECESSIVE, T CELL-NEGATIVE, B CELL- | |
| NEGATIVE, NK CELL-NEGATIVE, DUE | |
| TO ADENOSINE DEAMINASE | |
| DEFICIENCY | |
| SEVERE COMBINED | SEVERE COMBINED |
| IMMUNODEFICIENCY WITH | IMMUNODEFICIENCY WITH |
| MICROCEPHALY, GROWTH | SENSITIVITY TO IONIZING |
| RETARDATION, AND SENSITIVITY TO | RADIATION DUE TO NHEJ1 |
| IONIZING RADIATION | DEFICIENCY, INCLUDED |
| IMMUNODEFICIENCY 19; IMD19 | |
| T-CELL IMMUNODEFICIENCY WITH | |
| THYMIC APLASIA; TIDTA | |
| IL2-INDUCIBLE T-CELL KINASE; ITK | |
| SEVERE COMBINED | |
| IMMUNODEFICIENCY, AUTOSOMAL | |
| RECESSIVE, T CELL-NEGATIVE, B CELL- | |
| POSITIVE, NK CELL-NEGATIVE | |
| SEVERE COMBINED | |
| IMMUNODEFICIENCY, AUTOSOMAL | |
| RECESSIVE, T CELL-NEGATIVE, B CELL- | |
| NEGATIVE, NK CELL-POSITIVE | |
| CD8 ANTIGEN, ALPHA POLYPEPTIDE; | T-CELL ANTIGEN LEU2, INCLUDED; |
| CD8A | LEU2, INCLUDED |
| IMMUNODEFICIENCY 17; IMD17 | |
| T-CELL RECEPTOR-ALPHA/BETA | |
| DEFICIENCY | |
| T-CELL IMMUNODEFICIENCY, | |
| RECURRENT INFECTIONS, AND | |
| AUTOIMMUNITY WITH OR WITHOUT | |
| CARDIAC MALFORMATIONS; TIIAC | |
| SEVERE COMBINED | |
| IMMUNODEFICIENCY, AUTOSOMAL | |
| RECESSIVE, T CELL-NEGATIVE, B CELL- | |
| POSITIVE, NK CELL-POSITIVE | |
| SEVERE COMBINED | |
| IMMUNODEFICIENCY, X-LINKED; | |
| SCIDX1 | |
| IMMUNODEFICIENCY 49; IMD49 | |
| IMMUNODEFICIENCY 10; IMD10 | |
| T-CELL ACUTE LYMPHOCYTIC | |
| LEUKEMIA 1; TAL1 | |
| SEVERE COMBINED | SEVERE COMBINED |
| IMMUNODEFICIENCY WITH | IMMUNODEFICIENCY, |
| SENSITIVITY TO IONIZING RADIATION | ATHABASKAN-TYPE, INCLUDED; |
| | SCIDA, INCLUDED |
| CHEMOKINE, CC MOTIF, LIGAND 5; | |
| CCL5 | |
| SLAM FAMILY, MEMBER 6; SLAMF6 | |
| LINKER FOR ACTIVATION OF T CELLS | |
| FAMILY, MEMBER 2; LAT2 | |
| CD247 ANTIGEN; CD247 | |
| T-BOX TRANSCRIPTION FACTOR 21; | |
| TBX21 | |
| ALPHA/BETA T-CELL LYMPHOPENIA | |
| WITH GAMMA/DELTA T-CELL | |
| EXPANSION, SEVERE | |
| CYTOMEGALOVIRUS INFECTION, AND | |
| AUTOIMMUNITY | |
| IMMUNODEFICIENCY WITH DEFECTIVE | |
| T-CELL RESPONSE TO INTERLEUKIN 1 | |
| GRANZYME A; GZMA | |
| T-CELL RECEPTOR-ASSOCIATED | |
| TRANSMEMBRANE ADAPTOR 1; TRAT1 | |
| CUTANEOUS T-CELL LYMPHOMA- | CUTANEOUS T-CELL LYMPHOMA- |
| ASSOCIATED ANTIGEN 1; CTAGE1 | ASSOCIATED ANTIGEN 2, |
| | INCLUDED; CTAGE2, INCLUDED |
| UBIQUITIN-ASSOCIATED AND SH3 | |
| DOMAIN-CONTAINING PROTEIN B; | |
| UBASH3B | |
| CYTOTOXIC AND REGULATORY T-CELL | |
| MOLECULE; CRTAM | |
| T-CELL LEUKEMIA, HOMEOBOX 1; TLX1 | |
| T-CELL LEUKEMIA/LYMPHOMA 1A; | |
| TCL1A | |
| TRANSCRIPTION FACTOR 7; TCF7 | |
| NUCLEAR FACTOR OF ACTIVATED T | |
| CELLS, CYTOPLASMIC, CALCINEURIN- | |
| DEPENDENT 2; NFATC2 | |
| GLYCOGEN STORAGE DISEASE XII; | |
| GSD12 | |
| CD3 ANTIGEN, DELTA SUBUNIT; CD3D | |
| IMMUNODEFICIENCY 48; IMD48 | |
| T-CELL RECEPTOR ALPHA CHAIN | |
| CONSTANT REGION; TRAC | |
| KRABBE DISEASE | |
| INTERLEUKIN 9; IL9 | |
| ANTIGEN CD28; CD28 | |
| IMMUNODEFICIENCY 9; IMD9 | |
| IMMUNODEFICIENCY 32B; IMD32B | |
| MYELIN AND LYMPHOCYTE PROTEIN; | |
| MAL | |
| T-CELL ANTIGEN CD7; CD7 | |
| SOLUTE CARRIER FAMILY 2 | |
| (FACILITATED GLUCOSE | |
| TRANSPORTER), MEMBER 1; SLC2A1 | |
| INTERLEUKIN 2; IL2 | |
| IMMUNODEFICIENCY 54; IMD54 | |
| T-CELL IMMUNODEFICIENCY, | |
| CONGENITAL ALOPECIA, AND NAIL | |
| DYSTROPHY; TIDAND | |
| IMMUNODEFICIENCY 14; IMD14 | |
| CD4 ANTIGEN; CD4 | |
| INDUCIBLE T-CELL COSTIMULATOR; | |
| ICOS | |
| B-CELL EXPANSION WITH NFKB AND T- | |
| CELL ANERGY; BENTA | |
| LEUKEMIA, ACUTE LYMPHOBLASTIC; | LEUKEMIA, ACUTE |
| ALL | LYMPHOBLASTIC, SUSCEPTIBILITY |
| | TO, 1, INCLUDED |
| SQUAMOUS CELL CARCINOMA | |
| ANTIGEN RECOGNIZED BY T CELLS 1; | |
| SART1 | |
| DERMATAN SULFATE EPIMERASE; DSE | |
| SQUAMOUS CELL CARCINOMA | |
| ANTIGEN RECOGNIZED BY T CELLS 3; | |
| SART3 | |
| T CELL-INTERACTING ACTIVATING | |
| RECEPTOR ON MYELOID CELLS 1; | |
| TARM1 | |
| IMMUNODEFICIENCY 21; IMD21 | |
| PYRUVATE KINASE DEFICIENCY OF | |
| RED CELLS | |
| ECTODERMAL DYSPLASIA AND | |
| IMMUNODEFICIENCY 2; EDAID2 | |
| T CELL IMMUNE REGULATOR 1; TCIRG1 | TIRC7, INCLUDED |
| NUCLEAR FACTOR OF ACTIVATED T | |
| CELLS, CYTOPLASMIC, CALCINEURIN- | |
| DEPENDENT 1; NFATC1 | |
| HEPATITIS A VIRUS CELLULAR | |
| RECEPTOR 1; HAVCR1 | |
| VERTEBRAL ANOMALIES AND | |
| VARIABLE ENDOCRINE AND T-CELL | |
| DYSFUNCTION; VETD | |
| INTERLEUKIN 22; IL22 | |
| DIPEPTIDYL PEPTIDASE IV; DPP4 | |
| T-CELL LEUKEMIA HOMEOBOX 3; TLX3 | |
| INTERLEUKIN 2 RECEPTOR, ALPHA; | |
| IL2RA | |
| T-CELL LYMPHOMA INVASION AND | |
| METASTASIS 1; TIAM1 | |
| BASIC LEUCINE ZIPPER | |
| TRANSCRIPTION FACTOR, ATF-LIKE 3; | |
| BATF3 | |
| THYMOCYTE SELECTION-ASSOCIATED | |
| PROTEIN; THEMIS | |
| INTERLEUKIN 27 RECEPTOR, ALPHA; | |
| IL27RA | |
| T-BOX TRANSCRIPTION FACTOR 19; | |
| TBX19 | |
| LINKER FOR ACTIVATION OF T CELLS; | |
| LAT | |
| SH2 DOMAIN PROTEIN 2A; SH2D2A | |
| NUCLEAR FACTOR OF ACTIVATED T | |
| CELLS, CYTOPLASMIC, CALCINEURIN- | |
| DEPENDENT 3; NFATC3 | |
| CD27 ANTIGEN; CD27 | |
| CD3 ANTIGEN, EPSILON SUBUNIT; CD3E | |
| NUCLEAR FACTOR OF ACTIVATED T | |
| CELLS, CYTOPLASMIC, CALCINEURIN- | |
| DEPENDENT 4; NFATC4 | |
| FK506-BINDING PROTEIN 4; FKBP4 | |
| IMMUNODEFICIENCY 32A; IMD32A | |
| T-BOX TRANSCRIPTION FACTOR T; | |
| TBXT | |
| LYMPHOID ENHANCER-BINDING | SEBACEOUS TUMORS, SOMATIC, |
| FACTOR 1; LEF1 | INCLUDED |
| INTELLECTUAL DEVELOPMENTAL | |
| DISORDER WITH SPEECH DELAY, | |
| DYSMORPHIC FACIES, AND T-CELL | |
| ABNORMALITIES; IDDSFTA | |
| T-CELL LYMPHOMA, SUBCUTANEOUS | |
| PANNICULITIS-LIKE; SPTCL | |
| T-CELL LYMPHOPENIA, INFANTILE, | |
| WITH OR WITHOUT NAIL DYSTROPHY, | |
| AUTOSOMAL DOMINANT; TLIND | |
| SPLENOMEGALY SYNDROME WITH | |
| SPLENIC GERMINAL CENTER | |
| HYPOPLASIA AND REDUCED | |
| CIRCULATING T HELPER CELLS | |
| T-CELL SUBGROUPS, NON-HLA-LINKED | |
| CD4/CD8 T-CELL RATIO | |
| CD69 ANTIGEN; CD69 | |
| HLA-G HISTOCOMPATIBILITY | |
| ANTIGEN, CLASS I; HLA-G | |
| HUMAN T-CELL LEUKEMIA VIRUS | |
| ENHANCER FACTOR; HTLF | |
| T-CELL TUMOR INVASION AND | |
| METASTASIS 1; TTIM1 | |
| FREQUENTLY REARRANGED IN | |
| ADVANCED T-CELL LYMPHOMAS 2; | |
| FRAT2 | |
| TAX1-BINDING PROTEIN 1; TAX1BP1 | |
| PLECKSTRIN HOMOLOGY-LIKE | |
| DOMAIN, FAMILY A, MEMBER 1; | |
| PHLDA1 | |
| MELAN A; MLANA | |
| INDUCIBLE T-CELL COSTIMULATOR | |
| LIGAND; ICOSLG | |
| UBIQUITIN-ASSOCIATED AND SH3 | |
| DOMAIN-CONTAINING PROTEIN A; | |
| UBASH3A | |
| HEDGEHOG ACYLTRANSFERASE; HHAT | |
| CD96 ANTIGEN; CD96 | |
| HEPATITIS A VIRUS CELLULAR | |
| RECEPTOR 2; HAVCR2 | |
| INTERLEUKIN ENHANCER-BINDING | |
| FACTOR 2; ILF2 | |
| INTERLEUKIN ENHANCER-BINDING | |
| FACTOR 3; ILF3 | |
| T-CELL LYMPHOMA/LEUKEMIA 1B; | |
| TCL1B | |
| T-CELL LEUKEMIA, HOMEOBOX 2; TLX2 | |
| T-CELL LEUKEMIA/LYMPHOMA 6; TCL6 | |
| G PROTEIN-COUPLED RECEPTOR 65; | |
| GPR65 | |
| NUCLEAR FACTOR OF ACTIVATED T | |
| CELLS 5; NFAT5 | |
| T-CELL LYMPHOMA INVASION AND | |
| METASTASIS 2; TIAM2 | |
| MATURE T-CELL PROLIFERATION 1; | |
| MTCP1 | |
| MALIGNANT T-CELL AMPLIFIED | |
| SEQUENCE 1; MCTS1 | |
| HYDROXYCARBOXYLIC ACID | |
| RECEPTOR 1; HCAR1 | |
| V-SET DOMAIN-CONTAINING T-CELL | |
| ACTIVATION INHIBITOR 1; VTCN1 | |
| CUTANEOUS T-CELL LYMPHOMA- | |
| ASSOCIATED ANTIGEN 3; CTAGE3 | |
| CUTANEOUS T-CELL LYMPHOMA- | |
| ASSOCIATED ANTIGEN 4; CTAGE4 | |
| TYROSINE 3- | |
| MONOOXYGENASE/TRYPTOPHAN 5- | |
| MONOOXYGENASE ACTIVATION | |
| PROTEIN, THETA ISOFORM; YWHAQ | |
| T-CELL ANTIGEN RECEPTOR, GAMMA | |
| SUBUNIT, ALTERNATE READING | |
| FRAME PROTEIN | |
| T-CELL ACTIVATION GTPase- | |
| ACTIVATING PROTEIN; TAGAP | |
| PTC7 PROTEIN PHOSPHATASE, S. | |
| CEREVISIAE, HOMOLOG OF; PPTC7 | |
| WD REPEAT-CONTAINING PROTEIN 36; | |
| WDR36 | |
| Na+/K+ TRANSPORTING ATPase- | TCBA1/SUSP1 FUSION GENE, |
| INTERACTING 2; NKAIN2 | INCLUDED |
| DEF6, MOUSE, HOMOLOG OF; DEF6 | |
| T-CELL IMMUNOGLOBULIN AND | |
| MUCIN DOMAINS-CONTAINING | |
| PROTEIN 4; TIMD4 | |
| RING FINGER PROTEIN 125; RNF125 | |
| PDZ-BINDING KINASE; PBK | |
| CORNICHON, DROSOPHILA, HOMOLOG | |
| OF, 1; CNIH1 | |
| INTEGRIN-ALPHA FG-GAP REPEAT- | |
| CONTAINING PROTEIN 1; ITFG1 | |
| T-CELL IMMUNORECEPTOR WITH | |
| IMMUNOGLOBULIN AND ITIM | |
| DOMAINS; TIGIT | |
| PRE-T-CELL RECEPTOR, ALPHA-CHAIN | |
| PRECURSOR; PTCRA | |
| CHEMOKINE, CC MOTIF, LIGAND 27; | |
| CCL27 | |
| CHEMOKINE, CXC MOTIF, LIGAND 11; | |
| CXCL11 | |
| T-CELL RECEPTOR ALPHA CHAIN | |
| VARIABLE GENE CLUSTER; TRAV@ | |
| T-CELL RECEPTOR ALPHA CHAIN | |
| JOINING GENE CLUSTER; TRAJ@ | |
| T-CELL RECEPTOR BETA CHAIN | |
| CONSTANT REGION 2; TRBC2 | |
| T-CELL RECEPTOR BETA CHAIN | |
| VARIABLE GENE CLUSTER; TRBV@ | |
| T-CELL RECEPTOR BETA CHAIN | |
| DIVERSITY GENE 1; TRBD1 | |
| T-CELL RECEPTOR BETA CHAIN | |
| DIVERSITY GENE 2; TRBD2 | |
| T-CELL RECEPTOR BETA CHAIN | |
| JOINING GENE CLUSTER; TRBJ@ | |
| T-CELL RECEPTOR GAMMA CHAIN | |
| CONSTANT REGION 2; TRGC2 | |
| T-CELL RECEPTOR GAMMA CHAIN | |
| VARIABLE GENE CLUSTER; TRGV@ | |
| T-CELL RECEPTOR GAMMA CHAIN | |
| JOINING GENE CLUSTER; TRGJ@ | |
| T-CELL RECEPTOR DELTA CHAIN | |
| VARIABLE GENE CLUSTER; TRDV@ | |
| T-CELL RECEPTOR DELTA CHAIN | |
| DIVERSITY GENE CLUSTER; TRDD@ | |
| T-CELL RECEPTOR DELTA CHAIN | |
| JOINING GENE CLUSTER; TRDJ@ | |
| V-SET IMMUNOREGULATORY | |
| RECEPTOR; VSIR | |
| KELCH REPEAT- AND BTB/POZ | |
| DOMAIN-CONTAINING PROTEIN 8; | |
| KBTBD8 | |
| ZINC FINGER PROTEIN 683; ZNF683 | |
| FAS-ASSOCIATED FACTOR FAMILY, | |
| MEMBER 2; FAF2 | |
| PROTEIN-TYROSINE PHOSPHATASE, | |
| NONRECEPTOR-TYPE, 2; PTPN2 | |
| LIM DOMAIN ONLY 2; LMO2 | |
| T-CELL ACTIVATION ANTIGEN p250; | |
| TP250 | |
| CD6 ANTIGEN; CD6 | |
| CD8 ANTIGEN, BETA POLYPEPTIDE; | |
| CD8B | |
| CD3 ANTIGEN, GAMMA SUBUNIT; CD3G | |
| T-CELL RECEPTOR DELTA CHAIN | |
| CONSTANT REGION; TRDC | |
| T-CELL ACUTE LYMPHOCYTIC | |
| LEUKEMIA 2; TAL2 | |
| T-CELL LEUKEMIA/LYMPHOMA 4; TCL4 | |
| LIM DOMAIN ONLY 1; LMO1 | |
| T-CELL RECEPTOR BETA CHAIN | |
| CONSTANT REGION 1; TRBC1 | |
| FK506-BINDING PROTEIN 1A; FKBP1A | |
| FK506-BINDING PROTEIN 2; FKBP2 | |
| FK506-BINDING PROTEIN 3; FKBP3 | |
| T-CELL RECEPTOR GAMMA CHAIN | |
| CONSTANT REGION 1; TRGC1 | |
| THY-1 T-CELL ANTIGEN; THY1 | |
| GTP-BINDING MITOGEN-INDUCED T- | |
| CELL PROTEIN; GEM | |
| T-CELL LEUKEMIA TRANSLOCATION- | |
| ASSOCIATED GENE; TCTA | |
| T-CELL TRANSCRIPTION FACTOR EB; | TFEB/ALPHA FUSION GENE, |
| TFEB | INCLUDED |
| NUCLEAR RECEPTOR SUBFAMILY 4, | |
| GROUP A, MEMBER 2; NR4A2 | |
| MIA SH3 DOMAIN ER EXPORT FACTOR | |
| 2; MIA2 | |
| NONCODING TRANSCRIPT IN T CELLS | |
| TRANSCRIPTION FACTOR 7-LIKE 2; | TCF7L2/VTI1A FUSION GENE, |
| TCF7L2 | INCLUDED |
| FREQUENTLY REARRANGED IN | |
| ADVANCED T-CELL LYMPHOMAS; | |
| FRAT1 | |
| LEUCINE-RICH REPEAT-CONTAINING | |
| PROTEIN 8B; LRRC8B | |
| CYTOTOXIC T LYMPHOCYTE- | CYTOTOXIC T LYMPHOCYTE- |
| ASSOCIATED 4; CTLA4 | ASSOCIATED 4, SOLUBLE |
| | ISOFORM, INCLUDED |
| INHIBITOR OF KAPPA LIGHT CHAIN | |
| GENE ENHANCER IN B CELLS, KINASE | |
| OF, BETA; IKBKB | |
| T-BOX TRANSCRIPTION FACTOR 5; | |
| TBX5 | |
| T-BOX TRANSCRIPTION FACTOR 3; | |
| TBX3 | |
| T-BOX TRANSCRIPTION FACTOR 1; | |
| TBX1 | |
| LEYDIG CELL HYPOPLASIA, TYPE I | LEYDIG CELL HYPOPLASIA, TYPE |
| | II, INCLUDED |
| HISTONE GENE CLUSTER 1, H1 HISTONE | |
| FAMILY, MEMBER T; HIST1H1T | |
| ECTODERMAL DYSPLASIA AND | |
| IMMUNODEFICIENCY 1; EDAID1 | |
| GRANULOMATOUS DISEASE, CHRONIC, | |
| AUTOSOMAL RECESSIVE, | |
| CYTOCHROME b-POSITIVE, TYPE I; | |
| CDG1 | |
| NEURAMINIDASE DEFICIENCY | SIALIDOSIS, TYPE I, INCLUDED |
| TESTICULAR GERM CELL TUMOR; | SEMINOMA, INCLUDED |
| TGCT | |
| ALPHA-METHYLACETOACETIC | |
| ACIDURIA | |
| GALACTOSIALIDOSIS; GSL | |
| T-BOX TRANSCRIPTION FACTOR 22; | |
| TBX22 | |
| NUCLEAR FACTOR KAPPA-B | |
| INHIBITOR, ALPHA; NFKBIA | |
| OROTIC ACIDURIA | OROTIC ACIDURIA WITHOUT |
| | MEGALOBLASTIC ANEMIA, |
| | INCLUDED; OAWA, INCLUDED |
| LEUKOCYTE ADHESION DEFICIENCY, | |
| TYPE III; LAD3 | |
| URIDINE 5-PRIME MONOPHOSPHATE | |
| HYDROLASE DEFICIENCY, HEMOLYTIC | |
| ANEMIA DUE TO | |
| RHIZOMELIC CHONDRODYSPLASIA | |
| PUNCTATA, TYPE 2; RCDP2 | |
| ACYL-CoA DEHYDROGENASE, SHORT- | |
| CHAIN, DEFICIENCY OF; ACADSD | |
| TRANSCOBALAMIN I DEFICIENCY | TRANSCOBALAMIN I DEFICIENCY |
| | WITH LACTOFERRIN DEFICIENCY, |
| | INCLUDED |
| PROPERDIN DEFICIENCY, X-LINKED; | PROPERDIN DEFICIENCY, TYPE II, |
| CFPD | INCLUDED |
| ACYL-CoA DEHYDROGENASE, | |
| MEDIUM-CHAIN, DEFICIENCY OF; | |
| ACADMD | |
| CORTICOSTERONE METHYLOXIDASE | |
| TYPE II DEFICIENCY | |
| CEREBRAL CREATINE DEFICIENCY | |
| SYNDROME 3; CCDS3 | |
| LEIGH SYNDROME; LS | LEIGH SYNDROME DUE TO |
| | MITOCHONDRIAL COMPLEX I |
| | DEFICIENCY, INCLUDED |
| NK cell Deficiency | |
| SEVERE COMBINED | SEVERE COMBINED |
| IMMUNODEFICIENCY WITH | IMMUNODEFICIENCY WITH |
| MICROCEPHALY, GROWTH | SENSITIVITY TO IONIZING |
| RETARDATION, AND SENSITIVITY TO | RADIATION DUE TO NHEJ1 |
| IONIZING RADIATION | DEFICIENCY, INCLUDED |
| SEVERE COMBINED | SCID DUE TO ADA DEFICIENCY, |
| IMMUNODEFICIENCY, AUTOSOMAL | DELAYED ONSET, INCLUDED |
| RECESSIVE, T CELL-NEGATIVE, B CELL- | |
| NEGATIVE, NK CELL-NEGATIVE, DUE | |
| TO ADENOSINE DEAMINASE | |
| DEFICIENCY | |
| IMMUNODEFICIENCY 19; IMD19 | |
| IMMUNODEFICIENCY 54; IMD54 | |
| SEVERE COMBINED | |
| IMMUNODEFICIENCY, AUTOSOMAL | |
| RECESSIVE, T CELL-NEGATIVE, B CELL- | |
| POSITIVE, NK CELL-NEGATIVE | |
| IMMUNODEFICIENCY 21; IMD21 | |
| SEVERE COMBINED | |
| IMMUNODEFICIENCY, AUTOSOMAL | |
| RECESSIVE, T CELL-NEGATIVE, B CELL- | |
| NEGATIVE, NK CELL-POSITIVE | |
| SEVERE COMBINED | |
| IMMUNODEFICIENCY, AUTOSOMAL | |
| RECESSIVE, T CELL-NEGATIVE, B CELL- | |
| POSITIVE, NK CELL-POSITIVE | |
| IMMUNODEFICIENCY 17; IMD17 | |
| IMMUNODEFICIENCY 49; IMD49 | |
| KILLER CELL IMMUNOGLOBULIN-LIKE | KILLER CELL IMMUNOGLOBULIN- |
| RECEPTOR, THREE DOMAINS, LONG | LIKE RECEPTOR, THREE DOMAINS, |
| CYTOPLASMIC TAIL, 1; KIR3DL1 | SHORT CYTOPLASMIC TAIL, 1, |
| | INCLUDED; KIR3DS1, INCLUDED |
| KILLER CELL IMMUNOGLOBULIN-LIKE | NK-ASSOCIATED TRANSCRIPT 2A, |
| RECEPTOR, TWO DOMAINS, LONG | INCLUDED; NKAT2A, INCLUDED |
| CYTOPLASMIC TAIL, 3; KIR2DL3 | |
| KILLER CELL IMMUNOGLOBULIN-LIKE | NK-ASSOCIATED TRANSCRIPT 4A, |
| RECEPTOR, THREE DOMAINS, LONG | INCLUDED; NKAT4A, INCLUDED |
| CYTOPLASMIC TAIL, 2; KIR3DL2 | |
| KILLER CELL IMMUNOGLOBULIN-LIKE | NK-ASSOCIATED TRANSCRIPT |
| RECEPTOR, TWO DOMAINS, SHORT | 5delIg1, INCLUDED; NKAT5delIg1, |
| CYTOPLASMIC TAIL, 2; KIR2DS2 | INCLUDED |
| IMMUNODEFICIENCY 32B; IMD32B | |
| SEVERE COMBINED | |
| IMMUNODEFICIENCY, X-LINKED; | |
| SCIDX1 | |
| SEVERE COMBINED | SEVERE COMBINED |
| IMMUNODEFICIENCY WITH | IMMUNODEFICIENCY, |
| SENSITIVITY TO IONIZING RADIATION | ATHABASKAN-TYPE, INCLUDED; |
| | SCIDA, INCLUDED |
| SLAM FAMILY, MEMBER 6; SLAMF6 | |
| IMMUNODEFICIENCY 55; IMD55 | |
| ADENOSINE DEAMINASE; ADA | |
| GLYCOGEN STORAGE DISEASE XII; | |
| GSD12 | |
| GATA-BINDING PROTEIN 2; GATA2 | |
| KILLER CELL IMMUNOGLOBULIN-LIKE | |
| RECEPTOR, TWO DOMAINS, LONG | |
| CYTOPLASMIC TAIL, 1; KIR2DL1 | |
| KILLER CELL IMMUNOGLOBULIN-LIKE | |
| RECEPTOR, TWO DOMAINS, LONG | |
| CYTOPLASMIC TAIL, 2; KIR2DL2 | |
| KILLER CELL IMMUNOGLOBULIN-LIKE | |
| RECEPTOR, TWO DOMAINS, SHORT | |
| CYTOPLASMIC TAIL, 3; KIR2DS3 | |
| KILLER CELL IMMUNOGLOBULIN-LIKE | |
| RECEPTOR, TWO DOMAINS, SHORT | |
| CYTOPLASMIC TAIL, 4; KIR2DS4 | |
| KILLER CELL IMMUNOGLOBULIN-LIKE | |
| RECEPTOR, TWO DOMAINS, SHORT | |
| CYTOPLASMIC TAIL, 5; KIR2DS5 | |
| KRABBE DISEASE | |
| IMMUNODEFICIENCY 10; IMD10 | |
| NK6, DROSOPHILA, HOMOLOG OF, 1; | |
| NKX6-1 | |
| NK6 HOMEOBOX 2; NKX6-2 | |
| T-CELL IMMUNODEFICIENCY WITH | |
| THYMIC APLASIA; TIDTA | |
| MINICHROMOSOME MAINTENANCE | |
| COMPLEX COMPONENT 4; MCM4 | |
| PYRUVATE KINASE DEFICIENCY OF | |
| RED CELLS | |
| IMMUNODEFICIENCY 63 WITH | |
| LYMPHOPROLIFERATION AND | |
| AUTOIMMUNITY; IMD63 | |
| IMMUNODEFICIENCY 20; IMD20 | |
| KILLER CELL LECTIN-LIKE RECEPTOR, | |
| SUBFAMILY K, MEMBER 1; KLRK1 | |
| IL2-INDUCIBLE T-CELL KINASE; ITK | |
| T-CELL RECEPTOR-ALPHA/BETA | |
| DEFICIENCY | |
| T-CELL IMMUNODEFICIENCY, | |
| RECURRENT INFECTIONS, AND | |
| AUTOIMMUNITY WITH OR WITHOUT | |
| CARDIAC MALFORMATIONS; TIIAC | |
| IMMUNODEFICIENCY 32A; IMD32A | |
| INHIBITOR OF KAPPA LIGHT | |
| POLYPEPTIDE GENE ENHANCER IN B | |
| CELLS, KINASE OF, GAMMA; IKBKG | |
| Fc FRAGMENT OF IgG, LOW AFFINITY | |
| IIIa, RECEPTOR FOR: FCGR3A | |
| ECTODERMAL DYSPLASIA AND | |
| IMMUNODEFICIENCY 1; EDAID1 | |
| INHIBITOR OF KAPPA LIGHT CHAIN | |
| GENE ENHANCER IN B CELLS, KINASE | |
| OF, BETA; IKBKB | |
| FABRY DISEASE | FABRY DISEASE, CARDIAC |
| | VARIANT, INCLUDED |
| CD244 ANTIGEN; CD244 | |
| KILLER CELL LECTIN-LIKE RECEPTOR, | NKG2A, INCLUDED |
| SUBFAMILY C, MEMBER 1; KLRC1 | |
| KILLER CELL LECTIN-LIKE RECEPTOR, | |
| SUBFAMILY G, MEMBER 1; KLRG1 | |
| KILLER CELL LECTIN-LIKE RECEPTOR | |
| F1; KLRF1 | |
| IMMUNODEFICIENCY 43; IMD43 | |
| SLAM FAMILY, MEMBER 7; SLAMF7 | |
| CD3 ANTIGEN, DELTA SUBUNIT; CD3D | |
| INTERLEUKIN 2; IL2 | |
| IMMUNODEFICIENCY 41 WITH | |
| LYMPHOPROLIFERATION AND | |
| AUTOIMMUNITY; IMD41 | |
| GINS COMPLEX SUBUNIT 1; GINS1 | GINS COMPLEX, INCLUDED |
| LEYDIG CELL HYPOPLASIA, TYPE I | LEYDIG CELL HYPOPLASIA, TYPE |
| | II, INCLUDED |
| INTERLEUKIN 6; IL6 | |
| INTERLEUKIN 12B; IL12B | |
| NATURAL CYTOTOXICITY TRIGGERING | |
| RECEPTOR 2; NCR2 | |
| NK3 HOMEOBOX 1; NKX3-1 | |
| AUTOINFLAMMATION, ANTIBODY | |
| DEFICIENCY, AND IMMUNE | |
| DYSREGULATION; APLAID | |
| RADIATION SENSITIVITY OF NATURAL | |
| KILLER ACTIVITY | |
| CD300 ANTIGEN-LIKE FAMILY, | |
| MEMBER B; CD300LB | |
| LYMPHOPROLIFERATIVE SYNDROME 2; | |
| LPFS2 | |
| NEURAMINIDASE DEFICIENCY | SIALIDOSIS, TYPE I, INCLUDED |
| TESTICULAR GERM CELL TUMOR; | SEMINOMA, INCLUDED |
| TGCT | |
| ALPHA-METHYLACETOACETIC | |
| ACIDURIA | |
| TRANSCOBALAMIN I DEFICIENCY | TRANSCOBALAMIN I DEFICIENCY |
| | WITH LACTOFERRIN DEFICIENCY, |
| | INCLUDED |
| GRANULOMATOUS DISEASE, CHRONIC, | |
| AUTOSOMAL RECESSIVE, | |
| CYTOCHROME b-POSITIVE, TYPE I; | |
| CDG1 | |
| GALACTOSIALIDOSIS; GSL | |
| ALPHA/BETA T-CELL LYMPHOPENIA | |
| WITH GAMMA/DELTA T-CELL | |
| EXPANSION, SEVERE | |
| CYTOMEGALOVIRUS INFECTION, AND | |
| AUTOIMMUNITY | |
| GRANZYME A; GZMA | |
| CYTOKINE-DEPENDENT | |
| HEMATOPOIETIC CELL LINKER; CLNK | |
| OROTIC ACIDURIA | OROTIC ACIDURIA WITHOUT |
| | MEGALOBLASTIC ANEMIA, |
| | INCLUDED; OAWA, INCLUDED |
| URIDINE 5-PRIME MONOPHOSPHATE | |
| HYDROLASE DEFICIENCY, HEMOLYTIC | |
| ANEMIA DUE TO | |
| RHIZOMELIC CHONDRODYSPLASIA | |
| PUNCTATA, TYPE 2; RCDP2 | |
| PROPERDIN DEFICIENCY, X-LINKED; | PROPERDIN DEFICIENCY, TYPE II, |
| CFPD | INCLUDED |
| CORTICOSTERONE METHYLOXIDASE | |
| TYPE II DEFICIENCY | |
| CEREBRAL CREATINE DEFICIENCY | |
| SYNDROME 3; CCDS3 | |
| ACYL-CoA DEHYDROGENASE, | |
| MEDIUM-CHAIN, DEFICIENCY OF; | |
| ACADMD | |
| IMMUNODEFICIENCY 64; IMD64 | |
| NUCLEAR FACTOR KAPPA-B | |
| INHIBITOR, ALPHA; NFKBIA | |
| LEIGH SYNDROME; LS | LEIGH SYNDROME DUE TO |
| | MITOCHONDRIAL COMPLEX I |
| | DEFICIENCY, INCLUDED |
| LESCH-NYHAN SYNDROME; LNS | HPRT DEFICIENCY, NEUROLOGIC |
| | VARIANT, INCLUDED |
| METHEMOGLOBINEMIA DUE TO | METHEMOGLOBINEMIA, TYPE I, |
| DEFICIENCY OF METHEMOGLOBIN | INCLUDED |
| REDUCTASE | |
| MITOCHONDRIAL COMPLEX I | |
| DEFICIENCY, NUCLEAR TYPE 1; | |
| MC1DN1 | |
| PYRUVATE DEHYDROGENASE E1- | LACTIC ACIDEMIA, THIAMINE- |
| ALPHA DEFICIENCY; PDHAD | RESPONSIVE, INCLUDED |
| COENZYME Q10 DEFICIENCY, | |
| PRIMARY, 1; COQ10D1 | |
| ERYTHROCYTOSIS, FAMILIAL, 8; ECYT8 | |
| HSD10 MITOCHONDRIAL DISEASE; | |
| HSD10MD | |
| CORTICOSTERONE METHYLOXIDASE | |
| TYPE I DEFICIENCY | |
| GYRATE ATROPHY OF CHOROID AND | |
| RETINA; GACR | |
| HYPERPHENYLALANINEMIA, BH4- | |
| DEFICIENT, C; HPABH4C | |
| MITOCHONDRIAL COMPLEX I | |
| DEFICIENCY, NUCLEAR TYPE 20; | |
| MC1DN20 | |
| COMPLEMENT COMPONENT 8 | |
| DEFICIENCY, TYPE II; C8D2 | |
| MANNOSE-BINDING LECTIN | |
| DEFICIENCY; MBLD | |
| ACYL-CoA DEHYDROGENASE, SHORT- | |
| CHAIN, DEFICIENCY OF; ACADSD | |
| HIGH MOLECULAR WEIGHT | KININOGEN DEFICIENCY, TOTAL, |
| KININOGEN DEFICIENCY | INCLUDED |
| CARNITINE PALMITOYLTRANSFERASE | |
| II DEFICIENCY, MYOPATHIC, STRESS- | |
| INDUCED | |
| CANAVAN DISEASE | |
| CEREBRAL CREATINE DEFICIENCY | |
| SYNDROME 2; CCDS2 | |
| CARNITINE DEFICIENCY, SYSTEMIC | |
| PRIMARY; CDSP | |
| B-CELL CLL/LYMPHOMA 2; BCL2 | LEUKEMIA/LYMPHOMA, B-CELL, 2, |
| | INCLUDED |
| LYMPHOPROLIFERATIVE SYNDROME 3; | |
| LPFS3 | |
| KILLER CELL IMMUNOGLOBULIN-LIKE | |
| RECEPTOR, TWO DOMAINS, LONG | |
| CYTOPLASMIC TAIL, 4; KIR2DL4 | |
| D-BIFUNCTIONAL PROTEIN | |
| DEFICIENCY | |
| 17-BETA HYDROXYSTEROID | POLYCYSTIC OVARY SYNDROME |
| DEHYDROGENASE III DEFICIENCY | DUE TO 17-KETOSTEROID |
| | REDUCTASE DEFICIENCY, |
| | INCLUDED |
| ADRENAL HYPERPLASIA, | 17,20-LYASE DEFICIENCY, |
| CONGENITAL, DUE TO 17-ALPHA- | ISOLATED, INCLUDED |
| HYDROXYLASE DEFICIENCY | |
| INTERLEUKIN 12A; IL12A | |
| T-CELL IMMUNODEFICIENCY, | |
| CONGENITAL ALOPECIA, AND NAIL | |
| DYSTROPHY; TIDAND | |
| KILLER CELL LECTIN-LIKE RECEPTOR, | |
| SUBFAMILY D, MEMBER 1; KLRD1 | |
| CD99 ANTIGEN, X CHROMOSOME; CD99 | |
| TAF1 RNA POLYMERASE II, TATA BOX- | TRANSCRIPTION FACTOR IID, |
| BINDING PROTEIN-ASSOCIATED | INCLUDED; TFIID, INCLUDED; |
| FACTOR, 250-KD; TAF1 | TF2D, INCLUDED |
| MYELOID CELL LEUKEMIA SEQUENCE | MYELOID CELL LEUKEMIA 1, |
| 1; MCL1 | LONG ISOFORM, INCLUDED; |
| | MCL1L, INCLUDED |
| CD8 ANTIGEN, ALPHA POLYPEPTIDE; | T-CELL ANTIGEN LEU2, INCLUDED; |
| CD8A | LEU2, INCLUDED |
| IMMUNODEFICIENCY 18; IMD18 | IMMUNODEFICIENCY 18, SEVERE |
| | COMBINED IMMUNODEFICIENCY |
| | VARIANT, INCLUDED |
| LINKER FOR ACTIVATION OF T CELLS; | |
| LAT | |
| INTERLEUKIN 22; IL22 | |
| KILLER CELL LECTIN-LIKE RECEPTOR, | |
| SUBFAMILY B, MEMBER 1; KLRB1 | |
| SUCCINYL-CoA:3-OXOACID-CoA | |
| TRANSFERASE DEFICIENCY; SCOTD | |
| LIPASE DEFICIENCY, COMBINED | |
| CARNITINE PALMITOYLTRANSFERASE | |
| II DEFICIENCY, LETHAL NEONATAL | |
| CARNITINE PALMITOYLTRANSFERASE | |
| II DEFICIENCY, INFANTILE | |
| MUCOPOLYSACCHARIDOSIS, TYPE II; | |
| MPS2 | |
| LEUKOCYTE ADHESION DEFICIENCY, | |
| TYPE III; LAD3 | |
| METACHROMATIC LEUKODYSTROPHY; | PSEUDOARYLSULFATASE A |
| MLD | DEFICIENCY, INCLUDED |
| LYSOSOMAL ACID LIPASE DEFICIENCY | WOLMAN DISEASE, INCLUDED |
| ADRENAL HYPERPLASIA, | HYPERANDROGENISM, |
| CONGENITAL, DUE TO 21- | NONCLASSIC TYPE, DUE TO 21- |
| HYDROXYLASE DEFICIENCY | HYDROXYLASE DEFICIENCY, |
| | INCLUDED |
| PORPHYRIA, ACUTE INTERMITTENT; | PORPHYRIA, ACUTE |
| AIP | INTERMITTENT, NONERYTHROID |
| | VARIANT, INCLUDED |
| IMMUNOGLOBULIN A DEFICIENCY 1; | |
| IGAD1 | |
| ANDROGEN INSENSITIVITY | |
| SYNDROME; AIS | |
| ICHTHYOSIS, X-LINKED; XLI | ICHTHYOSIS, X-LINKED, |
| | COMPLICATED, INCLUDED |
| ARGININOSUCCINIC ACIDURIA | |
| MULTIPLE ACYL-CoA | GLUTARIC ACIDEMIA IIA, |
| DEHYDROGENASE DEFICIENCY; MADD | INCLUDED; GA2A, INCLUDED |
| GLYCOGEN STORAGE DISEASE II; GSD2 | |
| C-TYPE LECTIN DOMAIN FAMILY 2, | |
| MEMBER D; CLEC2D | |
| KILLER CELL LECTIN-LIKE RECEPTOR, | |
| SUBFAMILY C, MEMBER 2; KLRC2 | |
| FAMILY WITH SEQUENCE SIMILARITY | |
| 26, MEMBER F; FAM26F | |
| SUSCEPTIBILITY TO LYSIS BY | |
| ALLOREACTIVE NATURAL KILLER | |
| CELLS; EC1 | |
| KILLER CELL LECTIN-LIKE RECEPTOR | |
| F2; KLRF2 | |
| GLANZMANN THROMBASTHENIA; GT | |
| ISOLATED GROWTH HORMONE | |
| DEFICIENCY, TYPE III, WITH | |
| AGAMMAGLOBULINEMIA; IGHD3 | |
| HISTIDINEMIA | |
| HYPERPHENYLALANINEMIA, BH4- | HYPERPHENYLALANINEMIA, BH4- |
| DEFICIENT, A; HPABH4A | DEFICIENT, DUE TO PARTIAL PTS |
| | DEFICIENCY, INCLUDED |
| HYPEROXALURIA, PRIMARY, TYPE I; | |
| HP1 | |
| VITAMIN D HYDROXYLATION- | |
| DEFICIENT RICKETS, TYPE 1A; VDDR1A | |
| PYRUVATE CARBOXYLASE | |
| DEFICIENCY | |
| MYOPATHY DUE TO MYOADENYLATE | |
| DEAMINASE DEFICIENCY; MMDD | |
| KILLER CELL IMMUNOGLOBULIN-LIKE | |
| RECEPTOR, TWO DOMAINS, LONG | |
| CYTOPLASMIC TAIL, 5A; KIR2DL5A | |
| INTERLEUKIN 32; IL32 | |
| FARBER LIPOGRANULOMATOSIS; | |
| FRBRL | |
| FRUCTOSE INTOLERANCE, | |
| HEREDITARY; HFI | |
| GLUTATHIONURIA | |
| DIHYDROLIPOAMIDE | |
| DEHYDROGENASE DEFICIENCY; DLDD | |
| HOLOCARBOXYLASE SYNTHETASE | |
| DEFICIENCY | |
| THROMBOPHILIA DUE TO PROTEIN C | PROTEIN C DEFICIENCY, |
| DEFICIENCY, AUTOSOMAL DOMINANT; | ACQUIRED, INCLUDED |
| THPH3 | |
| BLEEDING DISORDER, PLATELET-TYPE, | |
| 12; BDPLT12 | |
| T-BOX TRANSCRIPTION FACTOR 21; | |
| TBX21 | |
| T-CELL IMMUNORECEPTOR WITH | |
| IMMUNOGLOBULIN AND ITIM | |
| DOMAINS; TIGIT | |
| FACTOR XII DEFICIENCY | |
| HOMOCYSTINURIA DUE TO | MTHFR DEFICIENCY, |
| DEFICIENCY OF N(5,10)- | THERMOLABILE TYPE, INCLUDED |
| METHYLENETETRAHYDROFOLATE | |
| REDUCTASE ACTIVITY | |
| 3-HYDROXY-3-METHYLGLUTARYL-CoA | |
| LYASE DEFICIENCY; HMGCLD | |
| METHYLMALONYL-CoA EPIMERASE | METHYLMALONYL-CoA |
| DEFICIENCY | EPIMERASE DEFICIENCY WITH |
| | SEPIAPTERIN REDUCTASE |
| | DEFICIENCY, INCLUDED |
| AROMATIC L-AMINO ACID | |
| DECARBOXYLASE DEFICIENCY | |
| HEPARIN COFACTOR II DEFICIENCY | |
| GLUCOCORTICOID RESISTANCE, | |
| GENERALIZED; GCCR | |
| ADRENAL HYPERPLASIA, | |
| CONGENITAL, DUE TO STEROID 11- | |
| BETA-HYDROXYLASE DEFICIENCY | |
| PYRUVATE KINASE, LIVER AND RED | |
| BLOOD CELL; PKLR | |
| HEREDITARY LEIOMYOMATOSIS AND | |
| RENAL CELL CANCER; HLRCC | |
| T-CELL ACUTE LYMPHOCYTIC | |
| LEUKEMIA 1; TAL1 | |
| CD160 ANTIGEN; CD160 | |
| LYMPHOPROLIFERATIVE SYNDROME, | |
| X-LINKED, 2; XLP2 | |
| T-CELL ANTIGEN CD7; CD7 | |
| CD3 ANTIGEN, EPSILON SUBUNIT; CD3E | |
| NUCLEAR FACTOR KAPPA-B, SUBUNIT | NFKB p105, INCLUDED |
| 1; NFKB1 | |
| CD96 ANTIGEN; CD96 | |
| HEMATOPOIETIC CELL SIGNAL | |
| TRANSDUCER; HCST | |
| RETINOIC ACID EARLY TRANSCRIPT | |
| 1E; RAET1E | |
| KILLER CELL IMMUNOGLOBULIN-LIKE | |
| RECEPTOR, THREE DOMAINS, LONG | |
| CYTOPLASMIC TAIL, 3; KIR3DL3 | |
| KILLER CELL LECTIN-LIKE RECEPTOR, | |
| SUBFAMILY C, MEMBER 3; KLRC3 | |
| CASPASE 8 DEFICIENCY | |
| COPROPORPHYRIA, HEREDITARY; HCP | |
| GLYCOGEN STORAGE DISEASE III; | GLYCOGEN STORAGE DISEASE IIIa, |
| GSD3 | INCLUDED; GSD IIIa, INCLUDED |
| GRANULOMATOUS DISEASE, CHRONIC, | |
| AUTOSOMAL RECESSIVE, | |
| CYTOCHROME b-POSITIVE, TYPE II; | |
| CDG2 | |
| N-ACETYLGLUTAMATE SYNTHASE | |
| DEFICIENCY; NAGSD | |
| HYPERLIPOPROTEINEMIA, TYPE I | HIGH DENSITY LIPOPROTEIN |
| | CHOLESTEROL LEVEL |
| | QUANTITATIVE TRAIT LOCUS 11, |
| | INCLUDED; HDLCQ11, INCLUDED |
| BIOTINIDASE DEFICIENCY | |
| CARNITINE PALMITOYLTRANSFERASE | |
| I DEFICIENCY | |
| SCHINDLER DISEASE, TYPE I | ALPHA-N- |
| | ACETYLGALACTOSAMINIDASE |
| | DEFICIENCY, TYPE III, INCLUDED |
| ISOBUTYRYL-CoA DEHYDROGENASE | |
| DEFICIENCY; IBDD | |
| FACTOR XI DEFICIENCY | |
| COMPLEMENT COMPONENT 6 | COMPLEMENT COMPONENT 6 |
| DEFICIENCY; C6D | DEFICIENCY, SUBTOTAL, |
| | INCLUDED |
| COMPLEMENT COMPONENT 8 | |
| DEFICIENCY, TYPE I; C8D1 | |
| PANCREATIC LIPASE DEFICIENCY; | PANCREATIC COLIPASE |
| PNLIPD | DEFICIENCY, INCLUDED |
| VITAMIN K-DEPENDENT CLOTTING | |
| FACTORS, COMBINED DEFICIENCY OF, | |
| 1; VKCFD1 | |
| IMMUNODEFICIENCY, COMMON | |
| VARIABLE, 3; CVID3 | |
| COWDEN SYNDROME 1; CWS1 | LHERMITTE-DUCLOS DISEASE, |
| | INCLUDED; LDD, INCLUDED |
| CD226 ANTIGEN; CD226 | |
| SPERMATOGENIC FAILURE, Y-LINKED, | SERTOLI CELL-ONLY SYNDROME, |
| 1; SPGFY1 | TYPE II, INCLUDED |
| Treg cell Deficiency | |
| SEVERE COMBINED | SEVERE COMBINED |
| IMMUNODEFICIENCY WITH | IMMUNODEFICIENCY WITH |
| MICROCEPHALY, GROWTH | SENSITIVITY TO IONIZING |
| RETARDATION, AND SENSITIVITY TO | RADIATION DUE TO NHEJI |
| IONIZING RADIATION | DEFICIENCY, INCLUDED |
| SEVERE COMBINED | SCID DUE TO ADA DEFICIENCY, |
| IMMUNODEFICIENCY, AUTOSOMAL | DELAYED ONSET, INCLUDED |
| RECESSIVE, T CELL-NEGATIVE, B CELL- | |
| NEGATIVE, NK CELL-NEGATIVE, DUE | |
| TO ADENOSINE DEAMINASE | |
| DEFICIENCY | |
| IMMUNODEFICIENCY 19; IMD19 | |
| CD8 ANTIGEN, ALPHA POLYPEPTIDE; | T-CELL ANTIGEN LEU2, INCLUDED; |
| CD8A | LEU2, INCLUDED |
| SEVERE COMBINED | |
| IMMUNODEFICIENCY, AUTOSOMAL | |
| RECESSIVE, T CELL-NEGATIVE, B CELL- | |
| POSITIVE, NK CELL-NEGATIVE | |
| SEVERE COMBINED | |
| IMMUNODEFICIENCY, AUTOSOMAL | |
| RECESSIVE, T CELL-NEGATIVE, B CELL- | |
| NEGATIVE, NK CELL-POSITIVE | |
| IL2-INDUCIBLE T-CELL KINASE; ITK | |
| IMMUNODEFICIENCY 17; IMD17 | |
| T-CELL IMMUNODEFICIENCY WITH | |
| THYMIC APLASIA: TIDTA | |
| SEVERE COMBINED | |
| IMMUNODEFICIENCY, AUTOSOMAL | |
| RECESSIVE, T CELL-NEGATIVE, B CELL- | |
| POSITIVE, NK CELL-POSITIVE | |
| T-CELL RECEPTOR-ALPHA/BETA | |
| DEFICIENCY | |
| T-CELL IMMUNODEFICIENCY, | |
| RECURRENT INFECTIONS, AND | |
| AUTOIMMUNITY WITH OR WITHOUT | |
| CARDIAC MALFORMATIONS; TIIAC | |
| IMMUNODEFICIENCY 49; IMD49 | |
| T-CELL ACUTE LYMPHOCYTIC | |
| LEUKEMIA 1; TAL1 | |
| CHEMOKINE, CC MOTIF, LIGAND 5; | |
| CCL5 | |
| LINKER FOR ACTIVATION OF T CELLS | |
| FAMILY, MEMBER 2; LAT2 | |
| SLAM FAMILY, MEMBER 6; SLAMF6 | |
| IMMUNODEFICIENCY 10; IMD10 | |
| SEVERE COMBINED | |
| IMMUNODEFICIENCY, X-LINKED; | |
| SCIDX1 | |
| T-BOX TRANSCRIPTION FACTOR 21; | |
| TBX21 | |
| SEVERE COMBINED | SEVERE COMBINED |
| IMMUNODEFICIENCY WITH | IMMUNODEFICIENCY, |
| SENSITIVITY TO IONIZING RADIATION | ATHABASKAN-TYPE, INCLUDED; |
| | SCIDA, INCLUDED |
| ALPHA/BETA T-CELL LYMPHOPENIA | |
| WITH GAMMA/DELTA T-CELL | |
| EXPANSION, SEVERE | |
| CYTOMEGALOVIRUS INFECTION, AND | |
| AUTOIMMUNITY | |
| IMMUNODEFICIENCY WITH DEFECTIVE | |
| T-CELL RESPONSE TO INTERLEUKIN 1 | |
| GRANZYME A; GZMA | |
| T-CELL RECEPTOR-ASSOCIATED | |
| TRANSMEMBRANE ADAPTOR 1; TRAT1 | |
| CUTANEOUS T-CELL LYMPHOMA- | CUTANEOUS T-CELL LYMPHOMA- |
| ASSOCIATED ANTIGEN 1; CTAGE1 | ASSOCIATED ANTIGEN 2, |
| | INCLUDED; CTAGE2, INCLUDED |
| UBIQUITIN-ASSOCIATED AND SH3 | |
| DOMAIN-CONTAINING PROTEIN B; | |
| UBASH3B | |
| CYTOTOXIC AND REGULATORY T-CELL | |
| MOLECULE; CRTAM | |
| T-CELL LEUKEMIA, HOMEOBOX 1; TLX1 | |
| T-CELL LEUKEMIA/LYMPHOMA 1A; | |
| TCL1A | |
| TRANSCRIPTION FACTOR 7; TCF7 | |
| NUCLEAR FACTOR OF ACTIVATED T | |
| CELLS, CYTOPLASMIC, CALCINEURIN- | |
| DEPENDENT 2; NFATC2 | |
| INTERLEUKIN 22; IL22 | |
| GLYCOGEN STORAGE DISEASE XII; | |
| GSD12 | |
| KRABBE DISEASE | |
| INTERLEUKIN 9; IL9 | |
| MYELIN AND LYMPHOCYTE PROTEIN; | |
| MAL | |
| IMMUNODEFICIENCY 32B; IMD32B | |
| EXOSTOSIN-LIKE | |
| GLYCOSYLTRANSFERASE 3; EXTL3 | |
| IMMUNODEFICIENCY 54; IMD54 | |
| B-CELL EXPANSION WITH NFKB AND T- | |
| CELL ANERGY; BENTA | |
| LEUKEMIA, ACUTE LYMPHOBLASTIC; | LEUKEMIA, ACUTE |
| ALL | LYMPHOBLASTIC, SUSCEPTIBILITY |
| | TO, 1, INCLUDED |
| SQUAMOUS CELL CARCINOMA | |
| ANTIGEN RECOGNIZED BY T CELLS 1; | |
| SART1 | |
| DERMATAN SULFATE EPIMERASE; DSE | |
| SQUAMOUS CELL CARCINOMA | |
| ANTIGEN RECOGNIZED BY T CELLS 3; | |
| SART3 | |
| T CELL-INTERACTING ACTIVATING | |
| RECEPTOR ON MYELOID CELLS 1; | |
| TARM1 | |
| SOLUTE CARRIER FAMILY 2 | |
| (FACILITATED GLUCOSE | |
| TRANSPORTER), MEMBER 1; SLC2A1 | |
| PYRUVATE KINASE DEFICIENCY OF | |
| RED CELLS | |
| CD4 ANTIGEN; CD4 | |
| IMMUNODEFICIENCY 48; IMD48 | |
| INDUCIBLE T-CELL COSTIMULATOR; | |
| ICOS | |
| T CELL IMMUNE REGULATOR 1; TCIRG1 | TIRC7, INCLUDED |
| IMMUNODEFICIENCY 9; IMD9 | |
| CD3 ANTIGEN, DELTA SUBUNIT; CD3D | |
| T-CELL RECEPTOR ALPHA CHAIN | |
| CONSTANT REGION; TRAC | |
| ECTODERMAL DYSPLASIA AND | |
| IMMUNODEFICIENCY 2; EDAID2 | |
| CD247 ANTIGEN; CD247 | |
| T-CELL IMMUNODEFICIENCY, | |
| CONGENITAL ALOPECIA, AND NAIL | |
| DYSTROPHY; TIDAND | |
| VERTEBRAL ANOMALIES AND | |
| VARIABLE ENDOCRINE AND T-CELL | |
| DYSFUNCTION; VETD | |
| DIPEPTIDYL PEPTIDASE IV; DPP4 | |
| INTERLEUKIN 2; IL2 | |
| INTERLEUKIN 2 RECEPTOR, ALPHA; | |
| IL2RA | |
| HEPATITIS A VIRUS CELLULAR | |
| RECEPTOR 1; HAVCR1 | |
| T-CELL LEUKEMIA HOMEOBOX 3; TLX3 | |
| ANTIGEN CD28; CD28 | |
| T-CELL ANTIGEN CD7; CD7 | |
| NUCLEAR FACTOR OF ACTIVATED T | |
| CELLS, CYTOPLASMIC, CALCINEURIN- | |
| DEPENDENT 1; NFATC1 | |
| T-CELL LYMPHOMA INVASION AND | |
| METASTASIS 1; TIAM1 | |
| T-BOX TRANSCRIPTION FACTOR 19; | |
| TBX19 | |
| REGENERATING ISLET-DERIVED 1- | |
| ALPHA; REG1A | |
| IMMUNODEFICIENCY 14; IMD14 | |
| INTERLEUKIN 27 RECEPTOR, ALPHA; | |
| IL27RA | |
| SH2 DOMAIN PROTEIN 2A; SH2D2A | |
| BASIC LEUCINE ZIPPER | |
| TRANSCRIPTION FACTOR, ATF-LIKE 3; | |
| BATF3 | |
| THYMOCYTE SELECTION-ASSOCIATED | |
| PROTEIN; THEMIS | |
| CD27 ANTIGEN; CD27 | |
| CD3 ANTIGEN, EPSILON SUBUNIT; CD3E | |
| FK506-BINDING PROTEIN 4; FKBP4 | |
| LINKER FOR ACTIVATION OF T CELLS; | |
| LAT | |
| NUCLEAR FACTOR OF ACTIVATED T | |
| CELLS, CYTOPLASMIC, CALCINEURIN- | |
| DEPENDENT 3; NFATC3 | |
| NUCLEAR FACTOR OF ACTIVATED T | |
| CELLS, CYTOPLASMIC, CALCINEURIN- | |
| DEPENDENT 4; NFATC4 | |
| PROTEASOME ACTIVATOR SUBUNIT 3; | |
| PSME3 | |
| REGENERATING ISLET-DERIVED 3- | |
| GAMMA; REG3G | |
| REGENERATING ISLET-DERIVED | |
| FAMILY, MEMBER 4; REG4 | |
| IMMUNODEFICIENCY 21; IMD21 | |
| REGENERATING ISLET-DERIVED 1- | |
| BETA; REG1B | |
| IMMUNODEFICIENCY 32A; IMD32A | |
| T-BOX TRANSCRIPTION FACTOR T; | |
| TBXT | |
| INTERLEUKIN 17A; IL17A | |
| LYMPHOID ENHANCER-BINDING | SEBACEOUS TUMORS, SOMATIC, |
| FACTOR 1; LEF1 | INCLUDED |
| INTELLECTUAL DEVELOPMENTAL | |
| DISORDER WITH SPEECH DELAY, | |
| DYSMORPHIC FACIES, AND T-CELL | |
| ABNORMALITIES; IDDSFTA | |
| T-CELL LYMPHOMA, SUBCUTANEOUS | |
| PANNICULITIS-LIKE; SPTCL | |
| T-CELL LYMPHOPENIA, INFANTILE, | |
| WITH OR WITHOUT NAIL DYSTROPHY, | |
| AUTOSOMAL DOMINANT; TLIND | |
| SPLENOMEGALY SYNDROME WITH | |
| SPLENIC GERMINAL CENTER | |
| HYPOPLASIA AND REDUCED | |
| CIRCULATING T HELPER CELLS | |
| T-CELL SUBGROUPS, NON-HLA-LINKED | |
| CD4/CD8 T-CELL RATIO | |
| CD69 ANTIGEN; CD69 | |
| HLA-G HISTOCOMPATIBILITY | |
| ANTIGEN, CLASS I; HLA-G | |
| HUMAN T-CELL LEUKEMIA VIRUS | |
| ENHANCER FACTOR; HTLF | |
| T-CELL TUMOR INVASION AND | |
| METASTASIS 1; TTIM1 | |
| FREQUENTLY REARRANGED IN | |
| ADVANCED T-CELL LYMPHOMAS 2; | |
| FRAT2 | |
| TAX1-BINDING PROTEIN 1; TAX1BP1 | |
| PLECKSTRIN HOMOLOGY-LIKE | |
| DOMAIN, FAMILY A, MEMBER 1; | |
| PHLDA1 | |
| MELAN A; MLANA | |
| INDUCIBLE T-CELL COSTIMULATOR | |
| LIGAND; ICOSLG | |
| UBIQUITIN-ASSOCIATED AND SH3 | |
| DOMAIN-CONTAINING PROTEIN A; | |
| UBASH3A | |
| HEDGEHOG ACYLTRANSFERASE; HHAT | |
| CD96 ANTIGEN; CD96 | |
| HEPATITIS A VIRUS CELLULAR | |
| RECEPTOR 2; HAVCR2 | |
| INTERLEUKIN ENHANCER-BINDING | |
| FACTOR 2; ILF2 | |
| INTERLEUKIN ENHANCER-BINDING | |
| FACTOR 3; ILF3 | |
| T-CELL LYMPHOMA/LEUKEMIA 1B; | |
| TCL1B | |
| T-CELL LEUKEMIA, HOMEOBOX 2; TLX2 | |
| T-CELL LEUKEMIA/LYMPHOMA 6; TCL6 | |
| G PROTEIN-COUPLED RECEPTOR 65; | |
| GPR65 | |
| NUCLEAR FACTOR OF ACTIVATED T | |
| CELLS 5; NFAT5 | |
| T-CELL LYMPHOMA INVASION AND | |
| METASTASIS 2; TIAM2 | |
| MATURE T-CELL PROLIFERATION 1; | |
| MTCP1 | |
| MALIGNANT T-CELL AMPLIFIED | |
| SEQUENCE 1; MCTS1 | |
| HYDROXYCARBOXYLIC ACID | |
| RECEPTOR 1; HCAR1 | |
| V-SET DOMAIN-CONTAINING T-CELL | |
| ACTIVATION INHIBITOR 1; VTCN1 | |
| CUTANEOUS T-CELL LYMPHOMA- | |
| ASSOCIATED ANTIGEN 3; CTAGE3 | |
| CUTANEOUS T-CELL LYMPHOMA- | |
| ASSOCIATED ANTIGEN 4; CTAGE4 | |
| TYROSINE 3- | |
| MONOOXYGENASE/TRYPTOPHAN 5- | |
| MONOOXYGENASE ACTIVATION | |
| PROTEIN, THETA ISOFORM; YWHAQ | |
| T-CELL ANTIGEN RECEPTOR, GAMMA | |
| SUBUNIT, ALTERNATE READING | |
| FRAME PROTEIN | |
| T-CELL ACTIVATION GTPase- | |
| ACTIVATING PROTEIN; TAGAP | |
| PTC7 PROTEIN PHOSPHATASE, S. | |
| CEREVISIAE, HOMOLOG OF; PPTC7 | |
| WD REPEAT-CONTAINING PROTEIN 36; | |
| WDR36 | |
| Na+/K+ TRANSPORTING ATPase- | TCBA1/SUSP1 FUSION GENE, |
| INTERACTING 2; NKAIN2 | INCLUDED |
| DEF6, MOUSE, HOMOLOG OF; DEF6 | |
| T-CELL IMMUNOGLOBULIN AND | |
| MUCIN DOMAINS-CONTAINING | |
| PROTEIN 4; TIMD4 | |
| RING FINGER PROTEIN 125; RNF125 | |
| PDZ-BINDING KINASE; PBK | |
| CORNICHON, DROSOPHILA, HOMOLOG | |
| OF, 1; CNIH1 | |
| INTEGRIN-ALPHA FG-GAP REPEAT- | |
| CONTAINING PROTEIN 1; ITFG1 | |
| T-CELL IMMUNORECEPTOR WITH | |
| IMMUNOGLOBULIN AND ITIM | |
| DOMAINS; TIGIT | |
| PRE-T-CELL RECEPTOR, ALPHA-CHAIN | |
| PRECURSOR; PTCRA | |
| CHEMOKINE, CC MOTIF, LIGAND 27; | |
| CCL27 | |
| CHEMOKINE, CXC MOTIF, LIGAND 11; | |
| CXCL11 | |
| T-CELL RECEPTOR ALPHA CHAIN | |
| VARIABLE GENE CLUSTER; TRAV@ | |
| T-CELL RECEPTOR ALPHA CHAIN | |
| JOINING GENE CLUSTER; TRAJ@ | |
| T-CELL RECEPTOR BETA CHAIN | |
| CONSTANT REGION 2; TRBC2 | |
| T-CELL RECEPTOR BETA CHAIN | |
| VARIABLE GENE CLUSTER; TRBV@ | |
| T-CELL RECEPTOR BETA CHAIN | |
| DIVERSITY GENE 1; TRBD1 | |
| T-CELL RECEPTOR BETA CHAIN | |
| DIVERSITY GENE 2; TRBD2 | |
| T-CELL RECEPTOR BETA CHAIN | |
| JOINING GENE CLUSTER; TRBJ@ | |
| T-CELL RECEPTOR GAMMA CHAIN | |
| CONSTANT REGION 2: TRGC2 | |
| T-CELL RECEPTOR GAMMA CHAIN | |
| VARIABLE GENE CLUSTER; TRGV@ | |
| T-CELL RECEPTOR GAMMA CHAIN | |
| JOINING GENE CLUSTER; TRGJ@ | |
| T-CELL RECEPTOR DELTA CHAIN | |
| VARIABLE GENE CLUSTER; TRDV@ | |
| T-CELL RECEPTOR DELTA CHAIN | |
| DIVERSITY GENE CLUSTER; TRDD@ | |
| T-CELL RECEPTOR DELTA CHAIN | |
| JOINING GENE CLUSTER; TRDJ@ | |
| V-SET IMMUNOREGULATORY | |
| RECEPTOR; VSIR | |
| KELCH REPEAT- AND BTB/POZ | |
| DOMAIN-CONTAINING PROTEIN 8; | |
| KBTBD8 | |
| ZINC FINGER PROTEIN 683; ZNF683 | |
| FAS-ASSOCIATED FACTOR FAMILY, | |
| MEMBER 2; FAF2 | |
| PROTEIN-TYROSINE PHOSPHATASE, | |
| NONRECEPTOR-TYPE, 2; PTPN2 | |
| LIM DOMAIN ONLY 2; LMO2 | |
| T-CELL ACTIVATION ANTIGEN p250; | |
| TP250 | |
| CD6 ANTIGEN; CD6 | |
| CD8 ANTIGEN, BETA POLYPEPTIDE; | |
| CD8B | |
| CD3 ANTIGEN, GAMMA SUBUNIT; CD3G | |
| T-CELL RECEPTOR DELTA CHAIN | |
| CONSTANT REGION; TRDC | |
| T-CELL ACUTE LYMPHOCYTIC | |
| LEUKEMIA 2; TAL2 | |
| T-CELL LEUKEMIA/LYMPHOMA 4; TCL4 | |
| LIM DOMAIN ONLY 1; LMO1 | |
| T-CELL RECEPTOR BETA CHAIN | |
| CONSTANT REGION 1; TRBC1 | |
| FK506-BINDING PROTEIN 1A; FKBP1A | |
| FK506-BINDING PROTEIN 2; FKBP2 | |
| FK506-BINDING PROTEIN 3; FKBP3 | |
| T-CELL RECEPTOR GAMMA CHAIN | |
| CONSTANT REGION 1; TRGC1 | |
| THY-1 T-CELL ANTIGEN; THY1 | |
| GTP-BINDING MITOGEN-INDUCED T- | |
| CELL PROTEIN; GEM | |
| T-CELL LEUKEMIA TRANSLOCATION- | |
| ASSOCIATED GENE; TCTA | |
| T-CELL TRANSCRIPTION FACTOR EB; | TFEB/ALPHA FUSION GENE, |
| TFEB | INCLUDED |
| NUCLEAR RECEPTOR SUBFAMILY 4, | |
| GROUP A, MEMBER 2; NR4A2 | |
| MIA SH3 DOMAIN ER EXPORT FACTOR | |
| 2; MIA2 | |
| NONCODING TRANSCRIPT IN T CELLS | |
| TRANSCRIPTION FACTOR 7-LIKE 2; | TCF7L2/VTI1A FUSION GENE, |
| TCF7L2 | INCLUDED |
| FREQUENTLY REARRANGED IN | |
| ADVANCED T-CELL LYMPHOMAS; | |
| FRAT1 | |
| LEUCINE-RICH REPEAT-CONTAINING | |
| PROTEIN 8B; LRRC8B | |
| CYTOTOXIC T LYMPHOCYTE- | CYTOTOXIC T LYMPHOCYTE- |
| ASSOCIATED 4; CTLA4 | ASSOCIATED 4, SOLUBLE |
| | ISOFORM, INCLUDED |
| T-BOX TRANSCRIPTION FACTOR 3; | |
| TBX3 | |
| T-BOX TRANSCRIPTION FACTOR 1; | |
| TBX1 | |
| T-BOX TRANSCRIPTION FACTOR 5; | |
| TBX5 | |
| LEYDIG CELL HYPOPLASIA, TYPE I | LEYDIG CELL HYPOPLASIA, TYPE |
| | II, INCLUDED |
| HISTONE GENE CLUSTER 1, H1 HISTONE | |
| FAMILY, MEMBER T; HIST1H1T | |
| NEURAMINIDASE DEFICIENCY | SIALIDOSIS, TYPE I, INCLUDED |
| TESTICULAR GERM CELL TUMOR; | SEMINOMA, INCLUDED |
| TGCT | |
| ALPHA-METHYLACETOACETIC | |
| ACIDURIA | |
| GRANULOMATOUS DISEASE, CHRONIC, | |
| AUTOSOMAL RECESSIVE, | |
| CYTOCHROME b-POSITIVE, TYPE I; | |
| CDG1 | |
| GALACTOSIALIDOSIS; GSL | |
| T-BOX TRANSCRIPTION FACTOR 22; | |
| TBX22 | |
| OROTIC ACIDURIA | OROTIC ACIDURIA WITHOUT |
| | MEGALOBLASTIC ANEMIA, |
| | INCLUDED; OAWA, INCLUDED |
| URIDINE 5-PRIME MONOPHOSPHATE | |
| HYDROLASE DEFICIENCY, HEMOLYTIC | |
| ANEMIA DUE TO | |
| RHIZOMELIC CHONDRODYSPLASIA | |
| PUNCTATA, TYPE 2; RCDP2 | |
| TRANSCOBALAMIN I DEFICIENCY | TRANSCOBALAMIN I DEFICIENCY |
| | WITH LACTOFERRIN DEFICIENCY, |
| | INCLUDED |
| PROPERDIN DEFICIENCY, X-LINKED; | PROPERDIN DEFICIENCY, TYPE II, |
| CFPD | INCLUDED |
| ACYL-CoA DEHYDROGENASE, | |
| MEDIUM-CHAIN, DEFICIENCY OF; | |
| ACADMD | |
| NUCLEAR FACTOR KAPPA-B | |
| INHIBITOR, ALPHA; NFKBIA | |

TABLE 4
Screening for disease causing mutations in genes
- genes of interest (GOI) for neuronal cells

| Gene | Disease 1 | Disease 2 | Disease 3 |
|---|---|---|---|
| AARS | Spinal muscular | | |
| | atrophy | | |
| AARS2 | Mitochondrial | | |
| | disease | | |
| ABCB7 | Mitochondrial | | |
| | disease | | |
| ABCC8 | Lysosomal storage | | |
| | diseases | | |
| ABCD1 | Leukodystrophy | | |
| ACAD9 | Mitochondrial | | |
| | disease | | |
| ACO2 | Eye | | |
| ACTA1 | Neuromuscular | | |
| | disease | | |
| ACTB | Brain | Neuronal migration | |
| | Malformations | disorders | |
| ACTG1 | Brain | Neuronal migration | |
| | Malformations | disorders | |
| ACY1 | Lysosomal storage | | |
| | diseases | | |
| ADAMTSL2 | Lysosomal storage | | |
| | diseases | | |
| ADAR | Dystonia and | Leukodystrophy | |
| | Parkinsons | | |
| ADCY5 | Dystonia and | | |
| | Parkinsons | | |
| ADGRG1 | Brain | | |
| | Malformations | | |
| ADGRG1 P | Neuronal migration | | |
| | disorders | | |
| ADSL | Epilepsy | Lysosomal storage | |
| | | diseases | |
| AFG3L2 | Dystonia and | Eye | Mitochondrial |
| | Parkinsons | | disease |
| AGA | Lysosomal storage | | |
| | diseases | | |
| AHI1 | Brain | | |
| | Malformations | | |
| AIFM1 | Mitochondrial | | |
| | disease | | |
| AIMP1 | Leukodystrophy | | |
| AKT3 | Brain | Neuronal migration | |
| | Malformations | disorders | |
| ALDH5A1 | Epilepsy | Lysosomal storage | |
| | | diseases | |
| ALDH7A1 | Epilepsy | Lysosomal storage | |
| | | diseases | |
| ALG13 | Epilepsy | | |
| ALS2 | ALS or FTLD | | |
| AMPD2 | Brain | | |
| | Malformations | | |
| ANG | ALS or FTLD | | |
| ANO3 | Dystonia and | | |
| | Parkinsons | | |
| ANO5 | Neuromuscular | | |
| | disease | | |
| ANTXR1 | Eye | | |
| AP4B1 | Leukodystrophy | | |
| AP4E1 | Leukodystrophy | | |
| AP4M1 | Leukodystrophy | | |
| AP4S1 | Leukodystrophy | | |
| APOPT1 | Mitochondrial | Leukodystrophy | |
| | disease | | |
| APTX | Dystonia and | Eye | |
| | Parkinsons | | |
| ARFGEF2 | Brain | Neuronal migration | |
| | Malformations | disorders | |
| ARHGEF9 | Epilepsy | | |
| ARL13B | Brain | | |
| | Malformations | | |
| ARSA | Dystonia and | Lysosomal storage | |
| | Parkinsons | diseases | |
| ARSB | Lysosomal storage | | |
| | diseases | | |
| ARX | Brain | Epilepsy | Neuronal migration |
| | Malformations | | disorders |
| ASAH1 | Neuromuscular | Lysosomal storage | Spinal muscular |
| | disease | diseases | atrophy |
| ASNS | Epilepsy | | |
| ASPA | Leukodystrophy | | |
| ASPM | Brain | | |
| | Malformations | | |
| ATAD3A | Eye | | |
| ATM | Dystonia and | | |
| | Parkinsons | | |
| ATP13A2 | Dystonia and | | |
| | Parkinsons | | |
| ATP1A3 | Dystonia and | | |
| | Parkinsons | | |
| ATP5A1 | Mitochondrial | | |
| | disease | | |
| ATP5E | Mitochondrial | | |
| | disease | | |
| ATP6AP1 | Lysosomal storage | | |
| | diseases | | |
| ATP6AP2 | Dystonia and | Epilepsy | |
| | Parkinsons | | |
| ATP6V0A2 | Brain | Neuronal migration | |
| | Malformations | disorders | |
| ATP7A | Spinal muscular | | |
| | atrophy | | |
| ATP7B | Dystonia and | | |
| | Parkinsons | | |
| ATPAF2 | Mitochondrial | | |
| | disease | | |
| ATRX | Epilepsy | | |
| AUH | Mitochondrial | Eye | |
| | disease | | |
| B3GALNT2 | Brain | Neuronal migration | |
| | Malformations | disorders | |
| B4GAT1 | Brain | | |
| | Malformations | | |
| B9D1 | Brain | | |
| | Malformations | | |
| B9D2 | Brain | | |
| | Malformations | | |
| BAG3 | Neuromuscular | | |
| | disease | | |
| BBS (Bardet-biedl | Eye | | |
| syndrome) | | | |
| BCS1L | Mitochondrial | | |
| | disease | | |
| BICD2 | Neuromuscular | Spinal muscular | |
| | disease | atrophy | |
| BOLA3 | Mitochondrial | | |
| | disease | | |
| BRAT1 | Epilepsy | | |
| BSCL2 | Spinal muscular | | |
| | atrophy | | |
| C10ORF2 | Eye | | |
| C10orf2 (AKA | Dystonia and | | |
| TWNK) | Parkinsons | | |
| C12orf65 | Mitochondrial | Eye | |
| | disease | | |
| C19orf12 | Dystonia and | Mitochondrial | Eye |
| | Parkinsons | disease | |
| C5orf42 | Brain | | |
| | Malformations | | |
| CACNA1A | Dystonia and | Epilepsy | |
| | Parkinsons | | |
| CACNA1S | Neuromuscular | | |
| | disease | | |
| CAPN3 | Neuromuscular | | |
| | disease | | |
| CARS2 | Mitochondrial | | |
| | disease | | |
| CASK | Brain | Epilepsy | |
| | Malformations | | |
| CC2D2A | Brain | | |
| | Malformations | | |
| CCND2 | Brain | | |
| | Malformations | | |
| CDKL5 | Epilepsy | | |
| CEP104 | Brain | | |
| | Malformations | | |
| CEP120 | Brain | | |
| | Malformations | | |
| Cep290 | Eye | Brain | |
| | | Malformations | |
| CEP41 | Brain | | |
| | Malformations | | |
| CHCHD10 | ALS or FTLD | Spinal muscular | |
| | | atrophy | |
| CHD2 | Epilepsy | | |
| CHKB | Neuromuscular | | |
| | disease | | |
| CHMP1A | Brain | | |
| | Malformations | | |
| CHMP2B | ALS or FTLD | | |
| CHN1 | Eye | | |
| CHRNA2 | Epilepsy | | |
| CHRNA4 | Epilepsy | | |
| CHRNB2 | Epilepsy | | |
| CISD2 | Eye | | |
| CIT | Brain | | |
| | Malformations | | |
| CLCN1 | Neuromuscular | | |
| | disease | | |
| CLCN2 | Leukodystrophy | | |
| CLCN4 | Epilepsy | | |
| CLN3 | Epilepsy | Lysosomal storage | |
| | | diseases | |
| CLN5 | Epilepsy | Lysosomal storage | |
| | | diseases | |
| CLN6 | Epilepsy | Lysosomal storage | |
| | | diseases | |
| CLN8 | Epilepsy | Lysosomal storage | |
| | | diseases | |
| CLPB | Mitochondrial | | |
| | disease | | |
| CNL | Eye | | |
| CNTNAP2 | Epilepsy | | |
| COA5 | Mitochondrial | | |
| | disease | | |
| COA7 | Leukodystrophy | | |
| COASY | Dystonia and | Mitochondrial | |
| | Parkinsons | disease | |
| COL12A1 | Neuromuscular | | |
| | disease | | |
| COL4A1 | Neuronal migration | | |
| | disorders | | |
| COL4A2 | Neuronal migration | | |
| | disorders | | |
| COL6A1 | Neuromuscular | | |
| | disease | | |
| COL6A2 | Neuromuscular | | |
| | disease | | |
| COL6A3 | Neuromuscular | | |
| | disease | | |
| COQ2 | Mitochondrial | | |
| | disease | | |
| COQ4 | Mitochondrial | | |
| | disease | | |
| COQ6 | Mitochondrial | | |
| | disease | | |
| COQ7 | Mitochondrial | | |
| | disease | | |
| COQ8A | Mitochondrial | | |
| | disease | | |
| COQ9 | Mitochondrial | | |
| | disease | | |
| COX10 | Mitochondrial | | |
| | disease | | |
| COX14 | Mitochondrial | | |
| | disease | | |
| COX15 | Mitochondrial | | |
| | disease | | |
| COX20 | Mitochondrial | | |
| | disease | | |
| COX6B1 | Mitochondrial | | |
| | disease | | |
| COX8A | Mitochondrial | | |
| | disease | | |
| CP | Dystonia and | | |
| | Parkinsons | | |
| CRYAB | Neuromuscular | | |
| | disease | | |
| CSPP1 | Brain | | |
| | Malformations | | |
| CSTB | Epilepsy | | |
| CTC1 | Leukodystrophy | | |
| CTNNB1 | Epilepsy | | |
| CTNS | Lysosomal storage | | |
| | diseases | | |
| CTSA | Lysosomal storage | | |
| | diseases | | |
| CTSD | Epilepsy | Lysosomal storage | |
| | | diseases | |
| CTSF | Epilepsy | Lysosomal storage | |
| | | diseases | |
| CUL4B | Brain | | |
| | Malformations | | |
| CYC1 | Mitochondrial | | |
| | disease | | |
| CYP27A1 | Dystonia and | Leukodystrophy | |
| | Parkinsons | | |
| DAG1 | Neuromuscular | | |
| | disease | | |
| DARS2 | Mitochondrial | | |
| | disease | | |
| DCAF17 | Dystonia and | | |
| | Parkinsons | | |
| DCHS1 | Brain | | |
| | Malformations | | |
| DCTN1 | Spinal muscular | | |
| | atrophy | | |
| DCX | Brain | Neuronal migration | |
| | Malformations | disorders | |
| DDX3X | Epilepsy | | |
| DEGS1 | Leukodystrophy | | |
| DEPDC5 | Epilepsy | | |
| DES | Neuromuscular | | |
| | disease | | |
| DGUOK | Mitochondrial | | |
| | disease | | |
| DLAT | Dystonia and | Mitochondrial | |
| | Parkinsons | disease | |
| DMD | Neuromuscular | | |
| | disease | | |
| DNA2 | Mitochondrial | | |
| | disease | | |
| DNAJB2 | Neuromuscular | Spinal muscular | |
| | disease | atrophy | |
| DNAJB6 | Neuromuscular | | |
| | disease | | |
| DNAJC19 | Eye | | |
| DNAJC5 | Dystonia and | Epilepsy | Lysosomal storage |
| | Parkinsons | | diseases |
| DNAJC6 | Dystonia and | | |
| | Parkinsons | | |
| DNM1 | Epilepsy | | |
| DNM1L | Mitochondrial | Eye | |
| | disease | | |
| DYNC1H1 | Brain | Neuronal migration | Spinal muscular |
| | Malformations | disorders | atrophy |
| EARS2 | Mitochondrial | | |
| | disease | | |
| ECHS1 | Mitochondrial | | |
| | disease | | |
| EEF1A2 | Epilepsy | | |
| EIF2B1 | Leukodystrophy | | |
| EIF2B2 | Leukodystrophy | | |
| EIF2B3 | Leukodystrophy | | |
| EIF2B4 | Leukodystrophy | | |
| ELAC2 | Mitochondrial | | |
| | disease | | |
| EMX2 | Neuronal migration | | |
| | disorders | | |
| EPM2A | Epilepsy | | |
| EPRS | Leukodystrophy | | |
| ETHE1 | Mitochondrial | | |
| | disease | | |
| EXOSC3 | Brain | Spinal muscular | |
| | Malformations | atrophy | |
| EXOSC8 | Spinal muscular | | |
| | atrophy | | |
| FA2H | Dystonia and | Leukodystrophy | |
| | Parkinsons | | |
| FAM126A | Leukodystrophy | | |
| FARS2 | Mitochondrial | | |
| | disease | | |
| FASTKD2 | Mitochondrial | | |
| | disease | | |
| FAT4 | Brain | Neuronal migration | |
| | Malformations | disorders | |
| FBXL4 | Mitochondrial | | |
| | disease | | |
| FBXO38 | Spinal muscular | | |
| | atrophy | | |
| FBXO7 | Dystonia and | | |
| | Parkinsons | | |
| FDX1L | Mitochondrial | Leukodystrophy | |
| | disease | | |
| FDXR | Eye | | |
| FH | Neuronal migration | | |
| | disorders | | |
| FKRP | Neuromuscular | Brain | |
| | disease | Malformations | |
| FKTN | Brain | Neuronal migration | |
| | Malformations | disorders | |
| FKTN | Neuromuscular | | |
| | disease | | |
| FLNA | Brain | Epilepsy | Neuronal migration |
| | Malformations | | disorders |
| FLVCR2 | Neuronal migration | | |
| | disorders | | |
| FOLR1 | Epilepsy | Leukodystrophy | |
| FOXG1 | Epilepsy | | |
| FOXRED1 | Mitochondrial | | |
| | disease | | |
| FRMD7 | Eye | | |
| FRRS1L | Epilepsy | | |
| FTL | Dystonia and | | |
| | Parkinsons | | |
| FUCA1 | Lysosomal storage | | |
| | diseases | | |
| FUS | ALS or FTLD | | |
| GAA | Lysosomal storage | Neuromuscular | |
| | diseases | disease | |
| GABBR2 | Epilepsy | | |
| GABRA1 | Epilepsy | | |
| GABRB3 | Epilepsy | | |
| GABRG2 | Epilepsy | | |
| GALC | Lysosomal storage | | |
| | diseases | | |
| GALNS | Lysosomal storage | | |
| | diseases | | |
| GAMT | Epilepsy | | |
| GARS | Mitochondrial | Spinal muscular | |
| | disease | atrophy | |
| GATM | Epilepsy | | |
| GBA | Lysosomal storage | Dystonia and | |
| | diseases | Parkinsons | |
| GBE1 | Neuromuscular | | |
| | disease | | |
| GCDH | Dystonia and | | |
| | Parkinsons | | |
| GCH1 | Dystonia and | | |
| | Parkinsons | | |
| GFAP | Leukodystrophy | | |
| GFER | Mitochondrial | | |
| | disease | | |
| GFM1 | Mitochondrial | | |
| | disease | | |
| GFM2 | Mitochondrial | | |
| | disease | | |
| GLA | Lysosomal storage | | |
| | diseases | | |
| GLB1 | Lysosomal storage | | |
| | diseases | | |
| GLDC | Epilepsy | | |
| GLRA1 | Dystonia and | | |
| | Parkinsons | | |
| GMPPB | Brain | Neuromuscular | |
| | Malformations | disease | |
| GMPPB | Neuronal migration | | |
| | disorders | | |
| GNAL | Dystonia and | | |
| | Parkinsons | | |
| GNAO1 | Epilepsy | | |
| GNE | Lysosomal storage | Neuromuscular | |
| | diseases | disease | |
| GNPTAB | Lysosomal storage | | |
| | diseases | | |
| GNPTG | Lysosomal storage | | |
| | diseases | | |
| GNS | Lysosomal storage | | |
| | diseases | | |
| GOSR2 | Epilepsy | | |
| GPC3 | Lysosomal storage | | |
| | diseases | | |
| GPR143 | Eye | | |
| GPSM2 | Brain | Neuronal migration | |
| | Malformations | disorders | |
| GRIN1 | Epilepsy | | |
| GRIN2B | Epilepsy | | |
| GRN | ALS or FTLD | | |
| GTPBP3 | Mitochondrial | | |
| | disease | | |
| GUSB | Lysosomal storage | | |
| | diseases | | |
| GYG2 | Mitochondrial | | |
| | disease | | |
| HEPACAM | Leukodystrophy | | |
| HESX1 | Eye | | |
| HEXA | Lysosomal storage | Spinal muscular | |
| | diseases | atrophy | |
| HEXB | Lysosomal storage | | |
| | diseases | | |
| HGSNAT | Lysosomal storage | | |
| | diseases | | |
| HIBCH | Leukodystrophy | | |
| HMGCL | Mitochondrial | | |
| | disease | | |
| HNRNPA2B1 | ALS or FTLD | | |
| HSPB1 | Spinal muscular | | |
| | atrophy | | |
| HSPB3 | Spinal muscular | | |
| | atrophy | | |
| HSPB8 | Spinal muscular | | |
| | atrophy | | |
| HSPD1 | Leukodystrophy | | |
| HTRA1 | Leukodystrophy | | |
| HYAL1 | Lysosomal storage | | |
| | diseases | | |
| IARS2 | Mitochondrial | | |
| | disease | | |
| IBA57 | Mitochondrial | | |
| | disease | | |
| IDS | Lysosomal storage | | |
| | diseases | | |
| IDUA | Lysosomal storage | | |
| | diseases | | |
| IFT172 | Brain | | |
| | Malformations | | |
| IGHMBP2 | Neuromuscular | Spinal muscular | |
| | disease | atrophy | |
| INPP5E | Brain | | |
| | Malformations | | |
| IQSEC2 | Epilepsy | | |
| ISCA2 | Mitochondrial | | |
| | disease | | |
| ISPD | Brain | Neuromuscular | Neuronal migration |
| | Malformations | disease | disorders |
| KATNB1 | Brain | Neuronal migration | |
| | Malformations | disorders | |
| KCNA2 | Epilepsy | | |
| KCNB1 | Epilepsy | | |
| KCNC1 | Epilepsy | | |
| KCNMA1 | Dystonia and | Epilepsy | |
| | Parkinsons | | |
| KCNQ2 | Epilepsy | | |
| KCNQ3 | Epilepsy | | |
| KCNT1 | Epilepsy | | |
| KCTD7 | Epilepsy | | |
| KIAA0586 | Brain | | |
| | Malformations | | |
| KIF1BP | Brain | Neuronal migration | |
| | Malformations | disorders | |
| KIF21A | Eye | | |
| KIF2A | Brain | | |
| | Malformations | | |
| KIF5C | Brain | | |
| | Malformations | | |
| KIF7 | Brain | Neuronal migration | |
| | Malformations | disorders | |
| KMT2B | Dystonia and | | |
| | Parkinsons | | |
| L1CAM | Neuronal migration | | |
| | disorders | | |
| LAMA2 | Neuromuscular | Neuronal migration | |
| | disease | disorders | |
| LAMB1 | Brain | Neuronal migration | |
| | Malformations | disorders | |
| LAMC3 | Brain | Neuronal migration | |
| | Malformations | disorders | |
| LAMP2 | Lysosomal storage | | |
| | diseases | | |
| LARGE | Neuronal migration | | |
| | disorders | | |
| LARGE1 | Brain | | |
| | Malformations | | |
| LAS1L | Spinal muscular | | |
| | atrophy | | |
| LGI1 | Epilepsy | | |
| LIAS | Epilepsy | Mitochondrial | |
| | | disease | |
| LIPA | Lysosomal storage | | |
| | diseases | | |
| LIPT1 | Mitochondrial | | |
| | disease | | |
| LMNA | Neuromuscular | | |
| | disease | | |
| LMNB1 | Leukodystrophy | | |
| LMOD3 | Neuromuscular | | |
| | disease | | |
| LRPPRC | Mitochondrial | | |
| | disease | | |
| LRRK2 | Dystonia and | | |
| | Parkinsons | | |
| LYRM4 | Mitochondrial | | |
| | disease | | |
| LYRM7 | Mitochondrial | | |
| | disease | | |
| LYST | Lysosomal storage | | |
| | diseases | | |
| MACF1 | Neuronal migration | | |
| | disorders | | |
| MAGI2 | Epilepsy | | |
| MAN2B1 | Lysosomal storage | | |
| | diseases | | |
| MANBA | Lysosomal storage | | |
| | diseases | | |
| MAPT | ALS or FTLD | | |
| MARS2 | Dystonia and | Mitochondrial | |
| | Parkinsons | disease | |
| MATR3 | ALS or FTLD | | |
| MBD5 | Epilepsy | | |
| MCOLN1 | Dystonia and | Lysosomal storage | |
| | Parkinsons | diseases | |
| MECP2 | Epilepsy | | |
| MECR | Eye | | |
| MED12 | Neuronal migration | | |
| | disorders | | |
| MEF2C | Epilepsy | Neuronal migration | |
| | | disorders | |
| MEGF10 | Neuromuscular | | |
| | disease | | |
| MFF | Mitochondrial | | |
| | disease | | |
| MFN2 | Mitochondrial | Eye | |
| | disease | | |
| MFSD8 | Epilepsy | Lysosomal storage | |
| | | diseases | |
| MGME1 | Mitochondrial | | |
| | disease | | |
| MICU1 | Mitochondrial | Neuromuscular | |
| | disease | disease | |
| MKS1 | Brain | | |
| | Malformations | | |
| MPC1 | Mitochondrial | | |
| | disease | | |
| MPDZ | Neuronal migration | | |
| | disorders | | |
| MPV17 | Mitochondrial | | |
| | disease | | |
| MRE11A | Dystonia and | | |
| | Parkinsons | | |
| MRPL12 | Mitochondrial | | |
| | disease | | |
| MRPL3 | Mitochondrial | | |
| | disease | | |
| MRPL44 | Mitochondrial | | |
| | disease | | |
| MRPS16 | Mitochondrial | | |
| | disease | | |
| MRPS22 | Mitochondrial | | |
| | disease | | |
| MRPS7 | Mitochondrial | | |
| | disease | | |
| MT-ATP6 | Leukodystrophy | Eye | |
| MT-ATP8 | Leukodystrophy | Eye | |
| MT-CO1 | Leukodystrophy | Eye | |
| MT-CO2 | Leukodystrophy | Eye | |
| MT-CO3 | Leukodystrophy | Eye | |
| MT-CYB | Leukodystrophy | Eye | |
| MTFMT | Mitochondrial | | |
| | disease | | |
| MT-ND1 | Leukodystrophy | Eye | |
| MT-ND2 | Leukodystrophy | Eye | |
| MT-ND3 | Leukodystrophy | Eye | |
| MT-ND4 | Leukodystrophy | Eye | |
| MT-ND4L | Leukodystrophy | Eye | |
| MT-ND5 | Leukodystrophy | Eye | |
| MT-ND5 | Leukodystrophy | Eye | |
| MT-ND6 | Leukodystrophy | Eye | |
| MTO1 | Mitochondrial | | |
| | disease | | |
| MTPAP | Mitochondrial | Eye | |
| | disease | | |
| MT-RNR1 | Leukodystrophy | Eye | |
| MT-RNR2 | Leukodystrophy | Eye | |
| MT-TA | Leukodystrophy | Eye | |
| MT-TC | Leukodystrophy | Eye | |
| MT-TD | Leukodystrophy | Eye | |
| MT-TE | Leukodystrophy | Eye | |
| MT-TF | Leukodystrophy | Eye | |
| MT-TG | Leukodystrophy | Eye | |
| MT-TH | Leukodystrophy | Eye | |
| MT-TI | Leukodystrophy | Eye | |
| MT-TK | Leukodystrophy | Eye | |
| MT-TL1 | Leukodystrophy | Eye | |
| MT-TL2 | Leukodystrophy | Eye | |
| MT-TM | Leukodystrophy | Eye | |
| MT-TN | Leukodystrophy | Eye | |
| MT-TP | Leukodystrophy | Eye | |
| MT-TQ | Leukodystrophy | Eye | |
| MT-TR | Leukodystrophy | Eye | |
| MT-TS1 | Leukodystrophy | Eye | |
| MT-TS2 | Leukodystrophy | Eye | |
| MT-TT | Leukodystrophy | Eye | |
| MT-TV | Leukodystrophy | Eye | |
| MT-TW | Leukodystrophy | Eye | |
| MT-TY | Leukodystrophy | Eye | |
| MYH7 | Neuromuscular | | |
| | disease | | |
| Myo7a | Eye | | |
| MYOT | Neuromuscular | | |
| | disease | | |
| NAGA | Lysosomal storage | | |
| | diseases | | |
| NAGLU | Lysosomal storage | | |
| | diseases | | |
| NARS2 | Mitochondrial | | |
| | disease | | |
| NDE1 | Brain | Neuronal migration | |
| | Malformations | disorders | |
| NDUFA1 | Mitochondrial | | |
| | disease | | |
| NDUFA10 | Mitochondrial | | |
| | disease | | |
| NDUFA11 | Mitochondrial | | |
| | disease | | |
| NDUFA12 | Mitochondrial | | |
| | disease | | |
| NDUFA2 | Mitochondrial | | |
| | disease | | |
| NDUFA4 | Mitochondrial | | |
| | disease | | |
| NDUFA9 | Mitochondrial | | |
| | disease | | |
| NDUFAF1 | Mitochondrial | | |
| | disease | | |
| NDUFAF2 | Mitochondrial | | |
| | disease | | |
| NDUFAF3 | Mitochondrial | | |
| | disease | | |
| NDUFAF4 | Mitochondrial | | |
| | disease | | |
| NDUFAF5 | Mitochondrial | | |
| | disease | | |
| NDUFAF6 | Mitochondrial | | |
| | disease | | |
| NDUFAF7 | Mitochondrial | | |
| | disease | | |
| NDUFB11 | Mitochondrial | | |
| | disease | | |
| NDUFB3 | Mitochondrial | | |
| | disease | | |
| NDUFB9 | Mitochondrial | | |
| | disease | | |
| NDUFS1 | Mitochondrial | Eye | |
| | disease | | |
| NDUFS2 | Mitochondrial | | |
| | disease | | |
| NDUFS3 | Mitochondrial | | |
| | disease | | |
| NDUFS4 | Mitochondrial | | |
| | disease | | |
| NDUFS6 | Mitochondrial | | |
| | disease | | |
| NDUFS7 | Mitochondrial | | |
| | disease | | |
| NDUFS8 | Mitochondrial | | |
| | disease | | |
| NDUFV1 | Mitochondrial | | |
| | disease | | |
| NDUFV2 | Mitochondrial | | |
| | disease | | |
| NEB | Neuromuscular | | |
| | disease | | |
| NEDD4L | Brain | | |
| | Malformations | | |
| NEU1 | Lysosomal storage | | |
| | diseases | | |
| NFS1 | Mitochondrial | | |
| | disease | | |
| NFU1 | Mitochondrial | | |
| | disease | | |
| NGLY1 | Epilepsy | | |
| NHLRC1 | Epilepsy | | |
| NKX2-1 | Dystonia and | | |
| | Parkinsons | | |
| NKX6-2 | Leukodystrophy | | |
| NPC1 | Dystonia and | Lysosomal storage | |
| | Parkinsons | diseases | |
| NPC2 | Dystonia and | Lysosomal storage | |
| | Parkinsons | diseases | |
| NPHP1 | Brain | | |
| | Malformations | | |
| NPHP3 | Brain | | |
| | Malformations | | |
| NPRL3 | Epilepsy | | |
| NR2F1 | Epilepsy | Eye | |
| NRXN1 | Epilepsy | | |
| NSDHL | Neuronal migration | | |
| | disorders | | |
| NT5C2 | Leukodystrophy | | |
| NUBPL | Mitochondrial | | |
| | disease | | |
| OCLN | Brain | Neuronal migration | |
| | Malformations | disorders | |
| OFD1 | Brain | | |
| | Malformations | | |
| OPA1 | Eye | | |
| OPA3 | Eye | | |
| OPHN1 | Brain | | |
| | Malformations | | |
| OPTN | ALS or FTLD | | |
| OTX2 | Eye | | |
| P23H | Eye | | |
| PAFAH1B1 | Brain | Neuronal migration | |
| | Malformations | disorders | |
| PANK2 | Dystonia and | | |
| | Parkinsons | | |
| PARK2 | Dystonia and | | |
| | Parkinsons | | |
| PARK7 | Dystonia and | | |
| | Parkinsons | | |
| PARS2 | Mitochondrial | | |
| | disease | | |
| PAX6 A | Eye | | |
| PC | Mitochondrial | | |
| | disease | | |
| PCCA | Mitochondrial | | |
| | disease | | |
| PCCB | Mitochondrial | | |
| | disease | | |
| PCDH19 | Epilepsy | | |
| PDGFB | Dystonia and | | |
| | Parkinsons | | |
| PDHA1 | Mitochondrial | | |
| | disease | | |
| PDHB | Mitochondrial | | |
| | disease | | |
| PDHX | Mitochondrial | | |
| | disease | | |
| PDSS1 | Mitochondrial | | |
| | disease | | |
| PDSS2 | Mitochondrial | | |
| | disease | | |
| PET100 | Mitochondrial | | |
| | disease | | |
| PFN1 | ALS or FTLD | | |
| PHGDH | Neuronal migration | | |
| | disorders | | |
| PHKA1 | Neuromuscular | | |
| | disease | | |
| PHOX2A | Eye | | |
| PIGA | Epilepsy | | |
| PIGN | Epilepsy | | |
| PIGO | Epilepsy | | |
| PIGV | Epilepsy | | |
| PIK3CA | Brain | Neuronal migration | |
| | Malformations | disorders | |
| PIK3R2 | Brain | Neuronal migration | |
| | Malformations | disorders | |
| PINK1 | Dystonia and | | |
| | Parkinsons | | |
| PLA2G6 | Dystonia and | | |
| | Parkinsons | | |
| PLCB1 | Epilepsy | | |
| PLEC | Neuromuscular | | |
| | disease | | |
| PLEKHG5 | Neuromuscular | Spinal muscular | |
| | disease | atrophy | |
| PNKD | Dystonia and | | |
| | Parkinsons | | |
| PNKP | Dystonia and | | |
| | Parkinsons | | |
| PNPLA2 | Lysosomal storage | | |
| | diseases | | |
| PNPO | Epilepsy | | |
| PNPT1 | Mitochondrial | | |
| | disease | | |
| POLG | Dystonia and | Epilepsy | Mitochondrial |
| | Parkinsons | | disease |
| POLG2 | Mitochondrial | | |
| | disease | | |
| POLR3B | Dystonia and | | |
| | Parkinsons | | |
| POM1 | Brain | | |
| | Malformations | | |
| POMGNT1 | Neuromuscular | Brain | |
| | disease | Malformations | |
| POMGNT2 | Brain | Neuronal migration | |
| | Malformations | disorders | |
| POMK | Brain | | |
| | Malformations | | |
| POMT1 | Neuronal migration | | |
| | disorders | | |
| POMT2 | Brain | | |
| | Malformations | | |
| PPT1 | Epilepsy | | |
| PPT1 | Lysosomal storage | | |
| | diseases | | |
| PQBP1 | Brain | | |
| | Malformations | | |
| PRKRA | Dystonia and | | |
| | Parkinsons | | |
| PRPH | ALS or FTLD | | |
| PRPS1 | Eye | | |
| PRRT2 | Dystonia and | Epilepsy | |
| | Parkinsons | | |
| PRRT2 | Epilepsy | | |
| PSAP | Lysosomal storage | | |
| | diseases | | |
| PYCR2 | Leukodystrophy | | |
| QARS | Epilepsy | Mitochondrial | |
| | | disease | |
| RAB18 | Brain | Neuronal migration | |
| | Malformations | disorders | |
| RAB3GAP1 | Neuronal migration | | |
| | disorders | | |
| RAB3GAP2 | Brain | Neuronal migration | |
| | Malformations | disorders | |
| RARS | Mitochondrial | | |
| | disease | | |
| RARS2 | Brain | Mitochondrial | |
| | Malformations | disease | |
| REEP1 | Spinal muscular | | |
| | atrophy | | |
| RELN | Brain | Neuronal migration | |
| | Malformations | disorders | |
| RGRIP1L | Brain | | |
| | Malformations | | |
| RMND1 | Mitochondrial | | |
| | disease | | |
| RNASEH2A | Leukodystrophy | | |
| RNASEH2C | Leukodystrophy | | |
| RNASET2 | Leukodystrophy | | |
| RNF216 | Leukodystrophy | | |
| ROBO3 | Eye | | |
| RRM2B | Mitochondrial | Eye | |
| | disease | | |
| RTN4IP1 | Eye | | |
| RTTN | Brain | Neuronal migration | |
| | Malformations | disorders | |
| SALL4 | Eye | | |
| SAMHD1 | Leukodystrophy | | |
| SATB2 | Epilepsy | | |
| SCARB2 | Epilepsy | Lysosomal storage | |
| | | diseases | |
| SCN1A | Epilepsy | | |
| SCN1B | Epilepsy | | |
| SCN2A | Epilepsy | | |
| SCN8A | Epilepsy | | |
| SCO1 | Mitochondrial | | |
| | disease | | |
| SCO2 | Spinal muscular | | |
| | atrophy | | |
| SCP2 | Dystonia and | | |
| | Parkinsons | | |
| SDHA | Mitochondrial | | |
| | disease | | |
| SDHAF1 | Mitochondrial | | |
| | disease | | |
| SEPSECS | Brain | Neuronal migration | |
| | Malformations | disorders | |
| SERAC1 | Mitochondrial | | |
| | disease | | |
| SETX | ALS or FTLD | Eye | |
| SGCE | Dystonia and | | |
| | Parkinsons | | |
| SGSH | Lysosomal storage | | |
| | diseases | | |
| SLC13A5 | Epilepsy | | |
| SLC16A2 | Dystonia and | | |
| | Parkinsons | | |
| SLC17A5 | Lysosomal storage | | |
| | diseases | | |
| SLC19A3 | Epilepsy | | |
| SLC19A3 | Mitochondrial | | |
| | disease | | |
| SLC1A4 | Leukodystrophy | | |
| SLC20A2 | Dystonia and | | |
| | Parkinsons | | |
| SLC25A22 | Epilepsy | | |
| SLC25A4 | Mitochondrial | Eye | |
| | disease | | |
| SLC25A46 | Mitochondrial | Eye | |
| | disease | | |
| SLC2A1 | Dystonia and | Epilepsy | |
| | Parkinsons | | |
| SLC30A10 | Dystonia and | | |
| | Parkinsons | | |
| SLC38A8 | Eye | | |
| SLC52A3 | ALS or FTLD | | |
| SLC5A7 | Spinal muscular | | |
| | atrophy | | |
| SLC6A1 | Epilepsy | | |
| SLC6A3 | Dystonia and | | |
| | Parkinsons | | |
| SLC6A8 | Epilepsy | | |
| SLC9A6 | Epilepsy | | |
| SMC1A | Epilepsy | | |
| SMN1 | Spinal muscular | | |
| | atrophy | | |
| SMN2 | Spinal muscular | | |
| | atrophy | | |
| SMPD1 | Dystonia and | Lysosomal storage | |
| | Parkinsons | diseases | |
| SNCA | Dystonia and | | |
| | Parkinsons | | |
| SOX2 | Eye | | |
| SPAST | Mitochondrial | | |
| | disease | | |
| SPG7 | Mitochondrial | Eye | |
| | disease | | |
| SPR | Dystonia and | | |
| | Parkinsons | | |
| SPTAN1 | Epilepsy | | |
| SQSTM1 | ALS or FTLD | | |
| SRD5A3 | Brain | | |
| | Malformations | | |
| SRPX2 | Brain | Neuronal migration | |
| | Malformations | disorders | |
| STXBP1 | Epilepsy | | |
| SUCLA2 | Mitochondrial | | |
| | disease | | |
| SUCLG1 | Mitochondrial | | |
| | disease | | |
| SUMF1 | Lysosomal storage | Leukodystrophy | |
| | diseases | | |
| SURF1 | Mitochondrial | | |
| | disease | | |
| SYNGAP1 | Epilepsy | | |
| SYNJ1 | Dystonia and | | |
| | Parkinsons | | |
| SZT2 | Epilepsy | | |
| TACO1 | Mitochondrial | | |
| | disease | | |
| TAF15 | ALS or FTLD | | |
| TARDBP | ALS or FTLD | | |
| TARS2 | Mitochondrial | | |
| | disease | | |
| TBC1D20 | Brain | | |
| | Malformations | | |
| TBC1D24 | Epilepsy | | |
| TBCE | Spinal muscular | | |
| | atrophy | | |
| TBK1 | ALS or FTLD | | |
| TBL1XR1 | Epilepsy | | |
| TCF4 | Epilepsy | | |
| TCTN1 | Brain | | |
| | Malformations | | |
| TCTN2 | Brain | | |
| | Malformations | | |
| TCTN3 | Brain | | |
| | Malformations | | |
| TFAM | Mitochondrial | | |
| | disease | | |
| TH | Dystonia and | | |
| | Parkinsons | | |
| THAP1 | Dystonia and | | |
| | Parkinsons | | |
| TIMM8A | Dystonia and | Eye | Mitochondrial |
| | Parkinsons | | disease |
| TK2 | Mitochondrial | Eye | |
| | disease | | |
| TMEM126A | Eye | | |
| TMEM126B | Mitochondrial | | |
| | disease | | |
| TMEM138 | Brain | | |
| | Malformations | | |
| TMEM216 | Brain | | |
| | Malformations | | |
| TMEM231 | Brain | | |
| | Malformations | | |
| TMEM237 | Brain | | |
| | Malformations | | |
| TMEM5 | Brain | Neuronal migration | |
| | Malformations | disorders | |
| TMEM67 | Brain | | |
| | Malformations | | |
| TMEM70 | Mitochondrial | | |
| | disease | | |
| TOR1A | Dystonia and | | |
| | Parkinsons | | |
| TOR1AIP1 | Dystonia and | | |
| | Parkinsons | | |
| TPK1 | Dystonia and | Mitochondrial | |
| | Parkinsons | disease | |
| TPP1 | Epilepsy | Lysosomal storage | |
| | | diseases | |
| TRAPPC11 | Dystonia and | | |
| | Parkinsons | | |
| TREX1 | Leukodystrophy | | |
| TRIT1 | Mitochondrial | | |
| | disease | | |
| TRMT10C | Mitochondrial | | |
| | disease | | |
| TRNT1 | Mitochondrial | | |
| | disease | | |
| TRPV4 | Spinal muscular | | |
| | atrophy | | |
| TSC1 | Epilepsy | | |
| TSC2 | Epilepsy | | |
| TSEN15 | Brain | | |
| | Malformations | | |
| TSEN2 | Brain | | |
| | Malformations | | |
| TSEN34 | Brain | | |
| | Malformations | | |
| TSEN54 | Brain | | |
| | Malformations | | |
| TSFM | Mitochondrial | Eye | |
| | disease | | |
| TTC19 | Mitochondrial | | |
| | disease | | |
| TTC21B | Brain | | |
| | Malformations | | |
| TUBA1A | Brain | Neuronal migration | ALS or FTLD |
| | Malformations | disorders | |
| TUBA8 | Brain | Neuronal migration | |
| | Malformations | disorders | |
| TUBB | Brain | | |
| | Malformations | | |
| TUBB2A | Brain | Neuronal migration | |
| | Malformations | disorders | |
| TUBB2B | Brain | Neuronal migration | |
| | Malformations | disorders | |
| TUBB3 | Eye | Brain | Neuronal migration |
| | | Malformations | disorders |
| TUBB4A | Dystonia and | Leukodystrophy | Brain |
| | Parkinsons | | Malformations |
| TUBBG1 | Brain | | |
| | Malformations | | |
| TUBG1 | Neuronal migration | | |
| | disorders | | |
| TUFM | Mitochondrial | | |
| | disease | | |
| TWNK | Mitochondrial | | |
| | disease | | |
| TYMP | Mitochondrial | Eye | |
| | disease | | |
| UBA1 | Spinal muscular | | |
| | atrophy | | |
| UBE3A | Epilepsy | | |
| UBQLN2 | ALS or FTLD | | |
| UCHL1 | Eye | | |
| UQCC2 | Mitochondrial | | |
| | disease | | |
| UQCC3 | Mitochondrial | | |
| | disease | | |
| UQCRB | Mitochondrial | | |
| | disease | | |
| UQCRC2 | Mitochondrial | | |
| | disease | | |
| UQCRQ | Mitochondrial | | |
| | disease | | |
| Usherin | Eye | | |
| VAPB | ALS or FTLD | Spinal muscular | |
| | | atrophy | |
| VARS2 | Mitochondrial | | |
| | disease | | |
| VCP | ALS or FTLD | | |
| VLDLR | Brain | Neuronal migration | |
| | Malformations | disorders | |
| VPS13A | Dystonia and | | |
| | Parkinsons | | |
| VPS33A | Lysosomal storage | | |
| | diseases | | |
| VPS35 | Dystonia and | | |
| | Parkinsons | | |
| VPS53 | Brain | | |
| | Malformations | | |
| VRK1 | Brain | Spinal muscular | |
| | Malformations | atrophy | |
| WDR45 | Dystonia and | Epilepsy | Mitochondrial |
| | Parkinsons | | disease |
| WDR62 | Brain | Neuronal migration | |
| | Malformations | disorders | |
| WFS1 | Eye | | |
| WWOX | Epilepsy | | |
| XPR1 | Dystonia and | | |
| | Parkinsons | | |
| YARS2 | Mitochondrial | | |
| | disease | | |
| YME1L1 | Eye | | |
| YWHAE | Neuronal migration | | |
| | disorders | | |
| ZEB2 | Epilepsy | | |
| ZFYVE26 | Leukodystrophy | | |
| ZNHIT3 | Eye | | |

In some embodiments, the at least one cell parameter comprises target cell yield after cell differentiation.

In some embodiments, the cell or the population of cells are T cells.

In some embodiments, the T cells are stem cell derived T cells

In some embodiments, the T cells are primary T cells.

In some embodiments, the T cells are CAR-T cells, optionally as described herein.

In some embodiments, the T cells are are CD3+ T cells, CD4+ T cells, CDS+ T cells, naïve T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T cells, effector memory T cells, effector memory T cells expressing CD45RA (TEMRA cells), tissue-resident memory (Trm) cells, virtual memory T cells, innate memory T cells, memory stem cell (Tse), γδ T cells, or a combination thereof.

In some embodiments, the T cells are cytotoxic T-cells, helper T-cells, memory T-cells, regulatory T-cells, tumor infiltrating lymphocytes, or a combination thereof.

In some embodiments, the T cell or the population of cells is categorised as exceptional or usable if the cell or the population of T cells shows a high amount of mucosal-associated invariant T (MAIT) relative to a reference cell or population of cells. In some embodiments, the T cell or the population of T cells is categorised as exceptional or usable if the cell or the population of cells shows a high amount of T Cell Receptor Beta Variable 28 (TRBV28) relative to a reference cell or population of cells. In some embodiments, the T cell or the population of T cells is categorised as exceptional or usable if the cell or the population of cells shows a high amount of Interleukin-17A (IL17A) relative to a reference cell or population of cells. In some embodiments, the T cell or the population of T cells is categorised as exceptional or usable if the cell or the population of cells is less activated and has a reduced NK-like signature relative to a reference cell or population of cells. In some embodiments, the T cell or the population of T cells is categorised as exceptional or usable if the cell or population of cells has an activated phenotype and/or Th1/Tc1 and Th17/Tc17 states.

In some embodiments, the at least one cell parameter comprises expression of one or markers associated with CD3+ T cells. CD4+ T cells. CDS+ T cells, naïve T cells, regulatory T (Treg) cells, non-regulatory T cells, Th1 cells, Th2 cells. Th9 cells, Th17 cells, T-follicular helper (Tfh) cells, cytotoxic T lymphocytes (CTL), effector T (Teff) cells, central memory T cells, effector memory T cells, effector memory T cells expressing CD45RA (TEMRA cells), tissue-resident memory (Trm) cells, virtual memory T cells, innate memory T cells, memory stem cell (Tse), γδ T cells. In some embodiments, the at least one cell parameter comprises expression of one or markers associated with memory stem cells. In some embodiments, the at least one cell parameter comprises expression of one or markers associated with central memory T cells.

In some embodiments, the subtype of cells are T cell subtypes. In some embodiments, the T cell subtypes comprise at least one of the T cell subtypes comprise at least one of the T cell subtypes selected from CD4 TN (naïve T), CD4 TSCM (stem memory), CD4 TCM (central memory), CD4 TEM (effector memory), CD4 TEFF (effector), CD4+CD27+, CD8 TN (naïve T), CD8 TSCM (stem memory), CD8 TCN (central memory), CD8 TEM (effector memory). CD8 TEFF (effector), CD8+CD27+.

In some embodiments, the at least one cell parameter is determination of a T cell subtype associated with good in vivo efficacy. In some embodiments, the at least one cell parameter is T cell subtype, such as CD4 TN (naïve T), CD4 TSCM (stem memory), CD4 TCM (central memory), CD4 TEM (effector memory), CD4 TEFF (effector), CD4+CD27+, CD8 TN (naïve T), CD8 TSCM (stem memory), CD8 TCN (central memory), CD8 TEM (effector memory). CD8 TEFF (effector), CD8+CD27+.

In some embodiments, the at least one cell parameter is determination of T cell subtype CD8+.

In some embodiments, the at least one cell parameter is determination of T cell subtype CD27+.

In some embodiments, the at least one cell parameter is determination of T cell subtype CD69−.

In some embodiments, the at least one cell parameter is determination of T cell subtype CD25−.In some embodiments, the at least one cell parameter is determination of T cell subtypes CD8+ and CD27+.

In some embodiments, the at least one cell parameter is determination of T cell subtypes CD8+ and CD69−.

In some embodiments, the at least one cell parameter is determination of T cell subtypes CD8+ and CD25−.

In some embodiments, the at least one cell parameter is determination of T cell subtypes CD27+ and CD69−.

In some embodiments, the at least one cell parameter is determination of T cell subtypes CD27+ and CD25−.

In some embodiments, the at least one cell parameter is determination of T cell subtypes CD69− and CD25−.

In some embodiments, the at least one cell parameter is determination of T cell subtypes CD8+, CD27+ and CD69−.

In some embodiments, the at least one cell parameter is determination of T cell subtypes CD8+, CD27+ and CD25−.

In some embodiments, the at least one cell parameter is determination of T cell subtypes CD8+, CD69− and CD25−.

In some embodiments, the at least one cell parameter is determination of T cell subtypes CD27+, CD69− and CD25−.

In some embodiments, the at least one cell parameter is determination of T cell subtypes CD8+, CD27+, CD69− and CD25−.

In some embodiments, the at least one cell parameter comprises determining expression of any of the afore-mentioned markers CD8+, CD27+, CD69− and/or CD25, or combination of these markers as disclosed herein, in the population of cells.

In some embodiments, the cells are NK cells.

In some embodiments, the NK cells are stem cell derived NK cells.

In some embodiments, the NK cells are primary NK cells.

In some embodiments, the NK cells are CAR-NK cells, optionally as described herein.

### Cells—Exemplary Parameters for Assaying NK Cells

In some embodiments, the at least one cell parameter includes production of factors modulating the function of other immune cells (e.g., interferon-y (IFN-γ), granulocyte macrophage colony-stimulating factor (GM-CSF); and/or chemokines (e.g., CCL1, CCL2, CCL3, CCL4, CCL5, and CXCL8).

In some embodiments, the at least one cell parameter comprises determining if the cell is CD56+ and CD16−; CD56− and CD16+; CD561o and CD16+; or CD56+ and CD3−.

In some embodiments, the cell or the population of cells are islet cells.

In some embodiments, the islet cells comprise up to 5, 10, 15, 20, 25, 35, or 40% alpha cells.

In some embodiments, the islet cells comprise up to 10, 15, 20, 25, 35, 40, 45, 50, 55, 60, 65, 70, 75, or 80% beta cells.

In some embodiments, the islet cells comprise up to 1, 2, 3, 4, 5, 6, 7, 8, 9, 10, 15, 20, or 25% delta cells.

In some embodiments, the cell or the population of cells are beta cells.

In some embodiments, the beta cells are stem cell derived

In some embodiments, the beta cells are primary beta cells.

### Cells—Exemplary Parameters for Assaying Beta Cells

In some embodiments, the at least one cell parameter comprises insulin production from beta cells (e.g., using an ELISA assay).

In some embodiments, the at least one cell parameter comprises Amylin or C-peptide production from beta cells.

In some embodiments, the cell or the population of cells are B cells.

In some embodiments, the B cells comprise precursor or immature B cells, naïve mature B cells, memory B cells, plasmablasts, and/or plasma cells.

In some embodiments, the B cells are CAR-B cells, optionally as described herein.

In some embodiments, the B cells are stem cell derived B cells.

In some embodiments, the B cells are primary B cells.

### Cells—Exemplary Parameters for Assaying B Cells

In some embodiments, the at least one cell parameter comprises determining antibody production by the cell or the population of cells.

In some embodiments, the at least one cell parameter comprises determining:
i. expression and/or secretion of certain cytokines, such as IFNγ, IL-2, IL-4, IL-6, IL-12 and TNFα;
ii. production and/or secretion of exogenous protein;
iii. expression of one or more of (such as all of) PAX5, BACH2, BCL-2, OBF1, OCT2, PU.1. SPIB, ETS1, and IRF8;
iv. expression of one or more of (such as all of) IRF4, BLIMP1, and XBP1;
v. expression of one or more of (such as all of) CD19, CD20, CD21, CD22, CD23, and CD24;
vi. expression of one or more of (such as all of) CD10, CD27, and CD38;
vii. expression of one or more of (such as all of) IRF4, BLIMP1, and XBP1;
viii. expression of one or more of (such as all of) PAX5, BACH2, BCL-2, OBF1, OCT2, PU.1, SPIB, ETS1, and IRF8;
ix. expression of one or more of (such as all of) CD19, CD38. CD27, CD269, and MHCII;
x. expression of one or more of (such as all of) CD20 and/or CD138;
xi. expression of one or more of (such as all of) IRF4, BLIMP1, and XBP1;
xii. expression of one or more of (such as all of) CXCR4, CD27, CD38, CD138, and CD269;
xiii. expression of one or more of (such as all of) CD19, CD20, and MHCII;
xiv. expression of one or more of (such as all of) CD19, CD20, CD40, CXCR4, CXCR5, and CXCR7; and/or
xv. cell surface levels of CD23 and/or CD38

In some embodiments, the cell or the population of cells are macrophages.

In some embodiments, the macrophages are CAR-macrophage cells, optionally as described herein.

In some embodiments, the macrophages are stem cell derived macrophages.

In some embodiments, the macrophages are primary macrophages.

### Cells—Exemplary Parameters for Assaying Macrophage Cells

In some embodiments, the at least one cell parameter comprises determining expression of M1 markers, such as HLA DR, CD86, CD80, and PDL1, and/or M2 markers, such as CD206, CD163; and/or determining targeted effector activity.

In some embodiments, the cell or the population of cells are hepatocytes.

In some embodiments, the hepatocytes are stem cell derived hepatocytes.

In some embodiments, the hepatocytes are primary hepatocytes.

### Cells—Exemplary Parameters for Assaying Hepatocyte Cells

In some embodiments, the at least one cell parameter comprises determining:
i. plateability;
ii. P450 induction;
iii. glycogen synthesis capability and/or storage capability;
iv. expression of one or more urea cycle pathway enzymes (e.g., using a ureagenesis assay);
v. presence or absence of a genetic aberration associated with a liver- associated monogenic disease;
vi. albumin secretion;
vii. exhibits α-1 antitrypsin (A1AT) secretion;
viii. coagulation Factor V secretion;
ix. lipid (e.g., VLDL, LDL, and HDL) uptake and/or storage capability;
x. indocyanine green (ICG) uptake and/or clearance capability;
xi. cytochrome p450 activity;
xii. asialoglycoprotein receptor expression (e.g., ASGR1 and/or ASGR2 expression;
xiii. alpha-fetoprotein (AFP) expression;
xiv. gamma-glutamyl transpeptidase activity;
xv. SOX9 expression;
xvi. keratin, type I cytoskeletal 18 (KRT18) expression;
xvii. HNF4A (e.g., HNF4a and HNF4α) expression;
xviii. G6PC expression;
xix. expression of hepatocyte markers HNF4α, ALB, CYP2C9;
xx. expression of the terminal differentiation marker PEPCK1;
xxi. expression of functional CYP enzymes, including CYP3A4, CYP2C9, CYP2B6, CYP1A2, CYP1A1, CYP2D6, CYP3A7, and CYP2E1;
xxii. expresses the markers HNF4a and ALB;
xxiii. functional glucose metabolism;
xxiv. functional lipid metabolism; and/or
xxv. expression of the terminal differentiation markers PEPCK1 and/or TAT.

In some embodiments, the cell or the population of cells are neural cells (e.g., cerebral endothelial cells; dopaminergic neurons, glial cells, and hematopoietic stem cells (HSCS) cells).

In some embodiments, the neural cells are glial cells.

In some embodiments, the neural cells are stem cell derived neural cells.

In some embodiments, the neural cells are primary neural cells.

### Cells—Exemplary Arameters for Assaying Neural e.g., Glial Cells

In some embodiments, the at least one cell parameter comprises determining:
i. expression of one or more markers selected from NKX2.2, PAX6, SOX10, brain derived neurotrophic factor BDNF, neutrotrophin-3 NT-3, NT-4, epidermal growth factor EGF, ciliary neurotrophic factor CNTF, nerve growth factor NGF, FGF8, EGFR, OLIG1, OLIG2, myelin basic protein MBP, GAP-43, LNGFR, nestin, GFAP, CD11b, CD11c, CD105, CX3CR1, P2RY12, IBA-1, TMEM119, and CD45;
ii. expression of one or more markers selected from A2B5, CD9, CD133, CD140a, FOXG1, GalC, GD3, GFAP, nestin, NG2, MBP, Musashi, 04, Olig1, Olig2, PDGFaR, SlO0P, glutamine synthetase, connexin 43, vimentin, BLBP, and GLAST;
iii. expression of one or more markers selected from PSA-NCAM, CD9, CD11, CD32, CD36, CD105, CD140a, nestin, and PDGFaR; and/or
iv. assaying cell morphology.

In some embodiments, the cell or the population of cells are cardiac cells.

In some embodiments, the cardiac cells are cardiomyocytes.

In some embodiments, the cardiac cells are stem cell derived cardiac cells.

In some embodiments, the cardiac cells are primary cardiac cells.

### Cells—Exemplary Parameters for Assaying Cardiac Cells e.g., Cardiomyocytes Cells

In some embodiments, the at least one cell parameter comprises determining:
i. assaying electrophysical potential;
ii. expression of one or more markers selected from Table 1;
iii. intrinsic beat rate below 90 beats per minute;
iv. action potential duration between 150-300 milliseconds;
v. ratios of expression levels of MYL2:MYL7, MYH7:MYH6, and TNNI3:TNN1 of at least 0.5:1 pre transplantation;
vi. ratios of expression levels of MYL2:MYL7, MYH7:MYH6, and TNNI3:TNN1 of at least 1:1 6 weeks post transplantation;
vii. average cell area at least 100 microns squared; and/or
viii. sarcomere content of at least 30%.

In some embodiments, the cell or the population of cells are ABO blood group type O.

In some embodiments, the cell or the population of cells comprise a functional ABO A allele and/or a functional ABO B allele.

In some embodiments, the cell or the population of cells are Rhesus factor negative (Rh−).

In some embodiments, the cell or population of cells are Rhesus factor positive (Rh+).

In some embodiments, the cell or the population of cells are CD34+ cells.

In some embodiments, the cell or population of cells are differentiated cells derived from CD34+ cells.

In some embodiments, the population of cells comprise different subtypes of cells. In some embodiments, the at least one parameter that is assayed is cell subtype.

In some embodiments, the subtype of cells are T cell subtypes. In some embodiments, the T cell subtypes comprise at least one of the T cell subtypes comprise at least one of the T cell subtypes selected from CD4 TN (naïve T). CD4 TSCM (stem memory). CD4 TCM (central memory), CD4 TEM (effector memory), CD4 TEFF (effector), CD4+CD27+, CD8 TN (naïve T), CD8 TSCM (stem memory), CD8 TCN (central memory), CD8 TEM (effector memory). CD8 TEFF (effector), CD8+CD27+.

In some embodiments, the method further comprises determining reduction of the functions of the MHC Class I and/or MHC class II molecules of the cells.

In some embodiments, determining reduction of the functions comprises determining reduction of the expression of the MHC Class I and/or MHC class II molecules.

In some embodiments, at least about 30% of cells in the population comprise modified cells.

In some embodiments:
i. the population includes cells with hypoimmune gene modifications (HIP cells) that exhibit reduced expression of one or more molecules of the MHC class I and/or MHC class II molecules and optionally also exhibit increased expression of at least one tolerogenic factor; and
ii. at least 30%, at least 40%, at least 50%, at least 60%, at least 70%, at least 80%, or at least 90% of cells in the population exhibit reduced expression of one or more molecules of the MHC class I and/or MHC class II molecules and optionally also exhibit increased expression of at least one tolerogenic factor.

In some embodiments, at least 5%, at least 10%, at least 20%, at least 30%, at least 40%, at least 50%, at least 60%, at least 70% of cells in the population of cells do not exhibit reduced expression of one or more molecules of the MHC class I and/or MHC class II molecules and optionally also do not exhibit increased expression of at least one tolerogenic factor.

In some embodiments, 30-90%, 30-80%, 30-70%, 30-60%, 30-50% or 40-50% of cells in the population of cells are HIP cells that exhibit reduced expression of one or more molecules of the MHC class I and/or MHC class II molecules and optionally increased expression of at least one tolerogenic factor.

In some embodiments, 70-100%, 80-100%, or 90-100% of cells in the population of cells express a CAR

In some embodiments, the cell or the population of cells, or progeny or differentiated cells derived from the cell or the population of cells have increased capability to evade NK cell mediated cytotoxicity upon administration to a subject as compared to a cell of the same type that does not comprise the one or more modifications.

In some embodiments, the cell or the population of cells, or progeny or differentiated cells derived from the cell or the population of cells undergo reduced cell lysis by mature NK cells upon administration to a subject as compared to a cell of the same type that does not comprise the one or more modifications.

In some embodiments, the cell or the population of cells, or progeny or differentiated cells derived from the cell or the population of cells induce a reduced immune response upon administration to a subject as compared to a cell of the same type that does not comprise the one or more modifications.

In some embodiments, the cell or the population of cells, or progeny or differentiated cells derived from the cell or the population of cells induce a reduced systemic inflammatory response upon administration to a subject as compared to a cell of the same type that does not comprise the one or more modifications.

In some embodiments, the cell or the population of cells, or progeny or differentiated cells derived from the cell or the population of cells induce a reduced local inflammatory response upon administration to a subject as compared to a cell of the same type that does not comprise the one or more modifications.

In some embodiments, the cell or the population of cells, or progeny or differentiated cells derived from the cell or the population of cells induce reduced complement pathway activation upon administration to a subject as compared to a cell of the same type that does not comprise the one or more modifications.

In some embodiments, the cell or the population of cells, or progeny or differentiated cells derived from the cell or the population of cells retain the ability to engraft and function upon administration to a subject.

In some embodiments, the cell or the population of cells, or progeny or differentiated cells derived from the cell or the population of cells has increased ability to engraft and function upon administration to a subject as compared to a cell of the same type that does not comprise the one or more modifications.

In one aspect, the present disclosure provides a cell or a population of cells having the features of the cell or the population of cells evaluated, profiled, identified or selected as described herein.

In one aspect, the present disclosure provides a cell having the features of a cell selected as described herein.

In one aspect, the present disclosure provides a population of cells having the features of a cell selected as described herein.

In one aspect, the present disclosure provides a cell or population of cells identified as described herein.

In one aspect, the present disclosure provides a cell or population of cells selected as described herein.

In some embodiments:
i. the population includes cells with hypoimmune gene modifications (HIP cells) that exhibit reduced expression of one or more molecules of the MHC class I and/or MHC class II molecules and optionally also exhibit increased expression of at least one tolerogenic factor; and
ii. at least 30%, at least 40%, at least 50%, at least 60%, at least 70%, at least 80%, or at least 90% of cells in the population exhibit reduced expression of one or more molecules of the MHC class I and/or MHC class II molecules and optionally also exhibit increased expression of at least one tolerogenic factor.

In some embodiments, at least 5%, at least 10%, at least 20%, at least 30%, at least 40%, at least 50%, at least 60%, at least 70% of cells in the population of cells do not exhibit reduced expression of one or more molecules of the MHC class I and/or MHC class II molecules and optionally also do not exhibit increased expression of at least one tolerogenic factor.

In some embodiments, 30-90%, 30-80%, 30-70%, 30-60%, 30-50% or 40-50% of cells in the population of cells are HIP cells that exhibit reduced expression of one or more molecules of the MHC class I and/or MHC class II molecules and optionally increased expression of at least one tolerogenic factor.

In some embodiments, 70-100%, 80-100%, or 90-100% of cells in the population of cells express a CAR.

In one aspect, the present disclosure provides a method of generating the cell as described herein comprising
obtaining a cell as described herein; and
introducing one or more modifications as described herein into the cell.

In some embodiments, the method further comprises selecting the cell from a population of cells based on the presence or expression level of one or more of the modifications.

In some embodiments, the cell is a stem cell or a progenitor cell and the method further comprises differentiating the stem cell or the progenitor cell.

In some embodiments, the cell is a stem cell and the method further comprises differentiating the stem cell.

In some embodiments, the cell is a pluripotent stem cell or a progeny thereof and the method comprises differentiating the pluripotent stem cell or progeny thereof.

In some embodiments, the cell is a primary cell.

In some embodiments, the method comprises introducing one or more gene edits into the genome of the cell.

In some embodiments, the one or more gene edits are introduced into the genome of the cell by non-targeted insertion.

In some embodiments, the one or more gene edits are introduced into the genome of the cell by targeted insertion.

In some embodiments, the one or more gene edits are introduced into one or more genes encoding the one or more molecules as described herein.

In some embodiments, the engineered cell has increased expression of the one or more molecules encoded by the one or more edited genes.

In some embodiments, the engineered cell has reduced expression of the one or more molecules encoded by the one or more edited genes.

In some embodiments, the one or more gene edits are introduced into the genome of cell using at least one of the genome editing complexes as described herein.

In some embodiments, the one or more gene edits are introduced into the genome of cell at one or more target genomic loci selected from the group consisting of an albumin gene locus, an ABO gene locus, a B2M gene locus, a CIITA gene locus, a CCR5 gene locus, a CD142 gene locus, a CLYBL gene locus, a CXCR4 gene locus, an F3 gene locus, a FUT1 gene locus, an HMGB1 gene locus, a KDM5D gene locus, an LRP1 gene locus, a MIC-A gene locus, a MIC-B gene locus, a PPP1R12C (also known as AAVS1) gene locus, an RHD gene locus, a ROSA26 gene locus, a safe harbor gene locus, a SHS231 locus, a TAP1 gene locus, a TRAC gene locus, and a TRBC gene locus.

In one aspect, the present disclosure provides a modified cell produced by the method as described herein.

In one aspect, the present disclosure provides a method of producing a composition comprising a cell or a population of cells comprising
a. obtaining a cell or a population of cells as described herein; and
b. formulating the composition comprising the cell or the population of cells.

In one aspect, the present disclosure provides a method of producing a composition comprising a cell or a population of cells comprising
a obtaining a cell or the population of cells as described herein;
b. introducing the one or more modifications described herein into the cell or the population of cells;
c. optionally selecting the modified cell or selecting the population of modified cells
   i. based on a level of the one or more modifications; and/or
   ii. the evaluation/profiling/identifying/selection methods as described herein; and
d. formulating the composition comprising the modified cell or the population of modified cells.

In one aspect, the present disclosure provides a method of producing a composition comprising the cell or the population of cells as described herein, comprising:
a. obtaining a cell or a population of cells as described herein
b. introducing the one or more modifications as described herein into the cell or the population of cells;
c. selecting the modified cell or selecting the population of modified cells:
   i. based on a level of the one or more modifications; and/or
   ii. the evaluation/profiling/identifying/selection methods as described herein; and
d. formulating the composition comprising the selected modified cell or the selected population of modified cells.

In some embodiments, selecting based on a level of the one or more modifications comprises selecting based on one or more modified molecules having reduced expression in the modified cell or the population of modified cells.

In some embodiments, selecting based on a level of the one or more modifications comprises selecting based on one or more modified molecules having increased expression in the modified cell or the population of modified cells.

In some embodiments, selecting based on a level of the one or more modifications comprises selecting based on cell surface expression of the one or more modified molecules as described herein.

In some embodiments, the method comprises formulating the composition in a pharmaceutically acceptable additive, carrier, diluent, or excipient.

In some embodiments, the pharmaceutically acceptable additive, carrier, diluent, or excipient comprises a pharmaceutically acceptable buffer.

In some embodiments, the pharmaceutically acceptable buffer comprises neutral buffer saline or phosphate buffered saline.

In some embodiments, the method comprises formulating the composition with Plasma-Lyte A®, dextrose, dextran, sodium chloride, human serum albumin (HSA), dimethylsulfoxide (DMSO), or a combination thereof.

In some embodiments, the method comprises formulating the composition with a cryoprotectant

In some embodiments, the method comprises formulating the composition in a serum-free cryopreservation medium comprising a cryoprotectant.

In some embodiments, the cryoprotectant comprises DMSO.

In some embodiments, the serum-free cryopreservation medium comprises about 5% to about 10% DMSO (v/v).

In some embodiments, the serum-free cryopreservation medium comprises about 10% DMSO (v/v). In some embodiments, the method further comprises storing the composition in a container.

In some embodiments, the method further comprises thawing the cell before step (b).

In some embodiments, the method further comprises freezing the modified cell, the population of modified cells, or the composition.

In some embodiments, the modified cell or the population of modified cells are frozen after step (b).

In some embodiments, the modified cell or the population of modified cells are thawed before step (c).

In some embodiments, the modified cell or the population of modified cells are frozen after step (c).

In some embodiments, the modified cell or the population of modified cells are thawed before step (d).

In some embodiments, the modified cell or the population of modified cells are frozen after step (c).

In some embodiments, the composition is frozen after step (d).

In one aspect, the present disclosure provides a composition comprising the cell or the population of cells as described herein or the modified cell or the population of modified cells as described herein.

In some embodiments:
i. the population includes cells with hypoimmune gene modifications (HIP cells) that exhibit reduced expression of one or more molecules of the MHC class I and/or MHC class II molecules and optionally also exhibit increased expression of at least one tolerogenic factor; and
i. at least 30%, at least 40%, at least 50%, at least 60%, at least 70%, at least 80%, or at least 90% of cells in the population exhibit reduced expression of one or more molecules of the MHC class I and/or MHC class II molecules and optionally also exhibit increased expression of at least one tolerogenic factor.

In some embodiments, at least 5%, at least 10%, at least 20%, at least 30%, at least 40%, at least 50%, at least 60%, at least 70% of cells in the population of cells do not exhibit reduced expression of one or more molecules of the MHC class I and/or MHC class II molecules and optionally also do not exhibit increased expression of at least one tolerogenic factor.

In some embodiments, 30-90%, 30-80%, 30-70%, 30-60%, 30-50% or 40-50% of cells in the population of cells are HIP cells that exhibit reduced expression of one or more molecules of the MHC class I and/or MHC class II molecules and optionally increased expression of at least one tolerogenic factor.

In some embodiments, 70-100%, 80-100%, or 90-100% of cells in the population of cells express a CAR.

In one aspect, the present disclosure provides a composition produced by the method as described herein.

In some embodiments, the composition as described herein, wherein the composition comprises a pharmaceutically acceptable additive, carrier, diluent, or excipient.

In some embodiments, the composition as described herein, wherein the composition is sterile.

In one aspect, the present disclosure provides a container comprising the composition as described herein.

In some embodiments, the container is a sterile bag.

In some embodiments, the sterile bag is a cryopreservation-compatible bag.

In one aspect, the present disclosure provides a kit comprising the composition as described herein or the container as described herein.

In some embodiments, the kit further comprises instructions for using the cells or the population of cells.

In one aspect, the present disclosure provides a method of making a cell therapy product comprising providing a cell or the population of cells that have been evaluated, profiled, identified, or selected according to a method as described herein and manufacturing a cell therapy product therefrom.

In some embodiments:
i. the population includes cells with hypoimmune gene modifications (HIP cells) that exhibit reduced expression of one or more molecules of the MHC class I and/or MHC class II molecules and optionally also exhibit increased expression of at least one tolerogenic factor; and
ii. at least 30%, at least 40%, at least 50%, at least 60%, at least 70%, at least 80%, or at least 90% of cells in the population exhibit reduced expression of one or more molecules of the MHC class I and/or MHC class II molecules and optionally also exhibit increased expression of at least one tolerogenic factor.

In some embodiments, at least 5%, at least 10%, at least 20%, at least 30%, at least 40%, at least 50%, at least 60%, at least 70% of cells in the population of cells do not exhibit reduced expression of one or more molecules of the MHC class I and/or MHC class II molecules and optionally also do not exhibit increased expression of at least one tolerogenic factor.

In some embodiments, 30-90%, 30-80%, 30-70%, 30-60%, 30-50% or 40-50% of cells in the population of cells are HIP cells that exhibit reduced expression of one or more molecules of the MHC class I and/or MHC class II molecules and optionally increased expression of at least one tolerogenic factor.

In some embodiments, 70-100%, 80-100%, or 90-100% of cells in the population of cells express a CAR.

In some embodiments, the method is performed by a third party.

In some embodiments, the manufacturing is performed by a third party.

In one aspect the present disclosure provides a method of evaluating, profiling, identifying or selecting a cell or a population of cells as described herein and manufacturing a cell therapy product therefrom.

A variety of uses of a cell or a population of cells are provided herein.

In one aspect the present disclosure provides the use of a cell or a population of cells that have been evaluated, profiled, identified or selected according to a method as described herein for manufacturing a cell therapy product.

In one aspect the present disclosure provides, a method of enhancing cell function of a cell therapy comprising evaluating, profiling, identifying, or selecting a cell or a population of cells as described herein and administering the cell therapy to a subject.

In one aspect the present disclosure provides a method of enhancing cell function of a cell therapy comprising evaluating, profiling, identifying or selecting a cell or a population of cells according to a method as described herein and making a cell therapy product therefrom and administering the cell therapy product to a subject.

In some embodiments, enhancing cell function comprises enhancing durability of cell response.

In some embodiments, enhancing cell function comprises promoting cell persistence.

In some embodiments, enhancing cell function comprises promoting engraftment.

In some embodiments, enhancing cell function comprises enhancing potency.

In some embodiments, enhancing cell function comprises enhancing hypoimmunogenicity.

In one aspect, the present disclosure provides a method of improving the likelihood of therapeutic effect of a cell therapy comprising evaluating, profiling, identifying or selecting a cell or a population of cells as described herein and administering the cell therapy to a subject.

In one aspect, the present disclosure provides a method of improving the likelihood of therapeutic effect of a cell therapy comprising evaluating, profiling, identifying or selecting a cell or a population of cells as described herein and making a cell therapy product therefrom and administering the cell therapy product to a subject.

In some embodiments, improving the likelihood of therapeutic effect comprises enhancing durability of response.

In some embodiments, improving the likelihood of therapeutic effect comprises promoting cell persistence.

In some embodiments, improving the likelihood of therapeutic effect comprises promoting engraftment.

In some embodiments, improving the likelihood of therapeutic effect comprises enhancing potency.

In some embodiments, improving the likelihood of therapeutic effect comprises enhancing hypoimmunogenicity.

In some aspects the present disclosure provides a method to determine whether to administer autologous cell therapy to a subject in need of cell therapy, the method comprising a method of profiling an autologous sample of cells from the subject as described herein, wherein the determination is made to administer autologous cell therapy to the subject if
i) the autologous sample of cells from the subject is categorised as above a reference value or at a reference value, particularly when categorised as above a reference value;
ii) the gene expression profile of the autologous sample of cells is comparable to a reference signature gene expression profile; and/or
iii) the AUC is less than 20 or between 20 and 100 at high dose, or the AUC is less than 11000 at low dose.

In some aspects the present disclosure provides, a method to determine whether to administer allogeneic cell therapy to a subject in need of cell therapy, the method comprising a method of profiling an autologous sample of cells from the subject as described herein, wherein the determination is made to administer allogeneic cell therapy to the subject if
i. the autologous sample of cells from the subject is categorised as below a reference value or at a reference value, particularly when categorised as below a reference value;
ii. the gene expression profile of the autologous sample of cells is not comparable to a reference signature gene expression profile; and/or
iii. the AUC is more than 100 at high dose, or the AUC is more than 11000 at low dose.

In some aspects the present disclosure provides a method to determine whether to administer in vivo CAR T therapy using a T cell targeted viral vector to a subject in need thereof, the method comprising a method of profiling an autologous sample of cells from the subject as described herein, wherein the determination is made to administer in vivo CAR T therapy using a T cell targeted viral vector to the subject if
i. the autologous sample of cells from the subject is categorised as below a reference value or at a reference value, particularly when categorised as below a reference value;
ii. the gene expression profile of the autologous sample of cells is not comparable to a reference signature gene expression profile; and/or
iii. the AUC is more than 100 at high dose, or the AUC is more than 11000 at low dose.

In some embodiments, the reference value is selected from the group consisting of: a reference value for predicted cell function, a reference value for defining the usability as a donor, a reference value for growth, a reference value for growth rate, a reference value for durability of cell growth, a reference value for durability of cell response, a reference value for cytokine production, a reference value for bulk cytokine production, a reference value for PSi, and a reference value for MSi.

In some embodiments, the reference value is selected from the group consisting of: an average value, a median value, a mean value, a value range, a pre-set value, a pre-determined value, an experimentally determined value, a computed value, and a cell type specific value.

In some embodiments, the reference value is determined in a reference cell or population of cells of the same cell type or subtypes as the cell or the population of cells.

In some embodiments, the reference value is determined in a reference cell or population of cells of a different cell type or subtypes as the cell or the population of cells.

In some embodiments, the reference value is determined in a reference cell or population of cells do not comprise the one or more modifications.

In some embodiments, the reference value is determined in a reference cell or population of cells comprise the one or more modifications.

In one aspect, the present disclosure provides use of an assay for evaluating cells for predicted function to predict the in vivo function of a cell or a population of cells for administration to a subject as a cell therapy.

In one aspect, the present disclosure provides use of an assay for evaluating cells for predicted function to predict the in vivo function of a cell or a population of cells for making a cell therapy product.

In some embodiments, the assay comprises at least one of an in vitro assay, an in vivo assay, an immune assay, a cell activity assay, a cell avidity assay, a cell proliferation assay, a cell cytotoxicity assay, a cellular stress assay, a tumor challenge assay, an expression assay, a cytokine production assay, a transcriptomic profiling assay, a proteomic profiling assay, a genomic profiling assay, a genomic stability assay, an epigenetic profiling assay, a cell developmental potential profiling assay, a cell subtyping assay; and/or a cell receptor profiling assay.

In some embodiments, the predicted cell function is at least one of the cell functions selected from the group consisting of: cell persistence, engraftment, durability of cell response, potency and hypoimmunogenicity.

In some embodiments, the predicted cell function is evaluated by assaying at least one of the cell parameters comprising:
a) cell activation;
b) cell polyfunctionality or cell multifunctionality;
c) cell cytotoxicity;
d) cell growth rate;
e) durability of cell growth;
f) durability of cell response;
g) the cell's ability to elicit adaptive and innate immune responses
h) characteristics associated with particular cell type (e.g., cell marker characterization, biomarker, intracellular markers, extracellular markers, cell cytokine production, antibody production);
i) cell cytokine production;
j) cell viability;
k) cell safety attributes;
l) cell impurity level(s);
m) immune cell identity;
n) immune cell subtyping;
o) cell subtype ratio;
p) cell proliferation;
q) HLA typing; and/or
r) transcriptomic profile.

In some embodiments, the assay isas described herein, the predicted cell function is as described herein and/or the cells areas described herein.

In one aspect, the present disclosure provides use of profiling the donor capability of a cell or the population of cells for categorising the cell or the population of cells for cell therapy.

In some embodiments, the profiling comprises evaluating cells for predicted function, wherein the evaluating is as described herein

In some embodiments, the profiling comprises a scale as described herein.

In some embodiments, the predicted cell function as described herein and/or the cells areas described herein.

Use of real-time quantitative live-cell analysis for measuring (i) growth rate and/or (ii) durability of cell growth and/or (iii) durability of cell response of a cell or the population of cells to predict the in vivo functionality of the cell or the population of cells for administration to a subject as a cell therapy.

In one aspect, the present disclosure provides use of a multiplex cytokine detection technique for measuring cytokine production for a panel of cytokines in a cell or the population of cells to predict the in vivo functionality of the cell or the population of cells as a cell therapy, optionally wherein the panel of cytokines comprises at least one (optionally all) of GM-CSF, GzmA, GzmB, IFNg, TNFa, Il2, Il6, Il17A, Il1b, and Il1RA.

In one aspect, the present disclosure provides use of single cell cytokine profiling on a cell or the population of cells to predict the in vivo functionality of the cell or the population of cells as a cell therapy.

In one aspect, the present disclosure provides use of gene expression profiling on a cell or the population of cells to determine resting state activation level to predict the in vivo functionality of the cell or the population of cells as a cell therapy.

In one aspect, the present disclosure provides use of gene expression profiling on a cell or the population of cells to determine activated state activation level to predict the in vivo functionality of the cell or the population of cells as a cell therapy.

In one aspect, the present disclosure provides a cell or a population of cells as described herein for use in therapy.

In one aspect, the present disclosure provides a composition comprising a cell or a population of cells as described herein, for use in therapy.

In one aspect, the present disclosure provides a cell or a population of cells as described herein, for use in a method of treating a disease or condition.

In one aspect, the present disclosure provides a composition as described herein, for use in a method of treating a disease or condition.

In some embodiments:
i. the population includes cells with hypoimmune gene modifications (HIP cells) that exhibit reduced expression of one or more molecules of the MHC class I and/or MHC class II molecules and optionally also exhibit increased expression of at least one tolerogenic factor; and
ii. at least 30%, at least 40%, at least 50%, at least 60%, at least 70%, at least 80%, or at least 90% of cells in the population exhibit reduced expression of one or more molecules of the MHC class I and/or MHC class II molecules and optionally also exhibit increased expression of at least one tolerogenic factor.

In some embodiments, at least 5%, at least 10%, at least 20%, at least 30%, at least 40%, at least 50%, at least 60%, at least 70% of cells in the population of cells do not exhibit reduced expression of one or more molecules of the MHC class I and/or MHC class II molecules and optionally also do not exhibit increased expression of at least one tolerogenic factor.

In some embodiments, 30-90%, 30-80%, 30-70%, 30-60%, 30-50% or 40-50% of cells in the population of cells are HIP cells that exhibit reduced expression of one or more molecules of the MHC class I and/or MHC class II molecules and optionally increased expression of at least one tolerogenic factor.

In some embodiments, 70-100%, 80-100%, or 90-100% of cells in the population of cells express a CAR.

Related to the profiling methods described herein, methods of subsequently making a cell therapy product are provided herein.

Related to the methods of making a cell therapy product described herein, methods of subsequently treating a disease or condition with the cell therapy product also are provided herein.

In one aspect, the present disclosure provides a method of treating a disease or condition in a subject comprising administering to the subject a cell or a population of cells selected as described herein.

In one aspect, the present disclosure provides a method of treating a disease or condition in a subject comprising administering to the subject a cell or a population of cells as described herein.

In one aspect, the present disclosure provides a method of treating a disease or condition in a subject comprising administering to the subject a composition as described herein.

In some embodiments, the subject is in need of therapy.

In some embodiments, the disease or condition is a cellular deficiency.

In some embodiments, the condition or disease is selected from the group consisting of diabetes, cancer, vascularization disorders, ocular disease, thyroid disease, skin diseases, and liver diseases.

In some embodiments, the condition or disease is associated with diabetes or is diabetes, optionally wherein the diabetes is Type I diabetes.

In some embodiments, the population of cells is a population of islet cells, including beta islet cells.

In some embodiments, the islet cells are selected from the group consisting of an islet progenitor cell, an immature islet cell, and a mature islet cell.

In some embodiments, the condition or disease is associated with a vascular condition or disease or is a vascular condition or disease.

In some embodiments, the cell or the population of cells comprises an endothelial cell.

In some embodiments, the condition or disease is associated with autoimmune thyroiditis or is autoimmune thyroiditis.

In some embodiments, the cell or the population of cells comprise a thyroid progenitor cell.

In some embodiments, the condition or disease is associated with a liver disease or is liver disease.

In some embodiments, the liver disease comprises cirrhosis of the liver.

In some embodiments, the cell or the population of cells comprise a hepatocyte or a hepatic progenitor cell.

In some embodiments, the condition or disease is associated with a corneal disease or is corneal disease.

In some embodiments, the corneal disease is Fuchs dystrophy or congenital hereditary endothelial dystrophy.

In some embodiments, the cell or the population of cells comprise a corneal endothelial progenitor cell or a corneal endothelial cells.

In some embodiments, the condition or disease is associated with a kidney disease or is kidney disease.

In some embodiments, the cell or the population of cells comprise a renal precursor cell or a renal cell.

In some embodiments, the disease or condition is a disease associated with cancer or cancer.

In some embodiments, the cancer is selected from the group consisting of B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.

In some embodiments, the cell or the population of cells comprise a T cell, an NK cell, or an NKT cell.

In some embodiments, the CAR is a CD19 CAR and the disease is CD19+ B-cell leukaemia.

In some embodiments, the condition or disease is associated with a hematopoietic disease or disorder or is a hematopoietic disease or disorder.

In some embodiments, the hematopoietic disease or disorder is myelodysplasia, aplastic anemia, Fanconi anemia, paroxysmal nocturnal hemoglobinuria, Sickle cell disease, Diamond Blackfan anemia, Schachman Diamond disorder, Kostmann's syndrome, chronic granulomatous disease, adrenoleukodystrophy, leukocyte adhesion deficiency, hemophilia, thalassemia, beta-thalassemia, leukaemia such as acute lymphocytic leukemia (ALL), acute myelogenous (myeloid)leukemia (AML), adult lymphoblastic leukaemia, chronic lymphocytic leukemia (CLL), B-cell chronic lymphocytic leukemia (B-CLL), chronic myeloid leukemia (CML), juvenile chronic myelogenous leukemia (CML), and juvenile myelomonocytic leukemia (JMML), severe combined immunodeficiency disease (SCID), X-linked severe combined immunodeficiency, Wiskott-Aldrich syndrome (WAS), adenosine-deaminase (ADA) deficiency, chronic granulomatous disease, Chediak-Higashi syndrome. Hodgkin's lymphoma, non-Hodgkin's lymphoma (NHL) or AIDS.

In some embodiments, the condition or disease is associated with leukemia or myeloma or is leukemia or myeloma.

In some embodiments, the condition or disease is associated with an autoimmune disease or condition or is an autoimmune disease or condition.

In some embodiments, the autoimmune disease or condition is acute disseminated encephalomyelitis, acute hemorrhagic leukoencephalitis, Addison's disease, Agammaglobulinemia, Alopecia areata, amyotrophic lateral sclerosis, ankylosing spondylitis, antiphospholipid syndrome, antisynthetase syndrome, atopic allergy, autoimmune aplastic anemia, autoimmune cardiomyopathy, autoimmune enteropathy, autoimmune hemolytic anemia, autoimmune hepatitis, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticaria, autoimmune uveitis, Balo disease, Balo concentric sclerosis, Bechets syndrome, Berger's disease, Bickerstaffs encephalitis, Blau syndrome, bullous pemphigoid, cancer, Castleman's disease, celiac disease, chronic inflammatory demyelinating polyneuropathy, chronic recurrent multifocal osteomyelitis, Churg-Strauss syndrome, cicatricial pemphigoid, Cogan syndrome, cold agglutinin disease, complement component 2 deficiency, cranial arteritis, CREST syndrome, Crohn's disease, Cushing's syndrome, cutaneous leukocytoclastic angiitis, Dego's disease, Dercum's disease, dermatitis herpetiformis, dermatomyositis, diabetes mellitus type I, diffuse cutaneous systemic sclerosis, Dressler's syndrome, discoid lupus erythematosus, eczema, enthesitis-related arthritis, eosinophilic fasciitis, eosinophilic gastroenteritis, epidermolysis bullosa acquisita, erythema nodosum, essential mixed cryoglobulinemia, Evan's syndrome, firodysplasia ossificans progressiva, fibrosing aveolitis, gastritis, gastrointestinal pemphigoid, giant cell arteritis, glomerulonephritis, goodpasture's syndrome, Grave's disease, Guillain-Barre syndrome (GBS), Hashimoto's encephalitis, Hashimoto's thyroiditis, hemolytic anaemia, Henoch-Schonlein purpura, herpes gestationis, hypogammaglobulinemia, idiopathic inflammatory demyelinating disease, idiopathic pulmonary fibrosis, idiopathic thrombocytopenic purpura, IgA nephropathy, inclusion body myositis, inflammatory demyelinating polyneuropathy, interstitial cystitis, juvenile idiopathic arthritis, juvenile rheumatoid arthritis, Kawasaki's disease, Lambert-Eaton myasthenic syndrome, leukocytoclastic vasculitis, lichen planus, lichen sclerosus, linear IgA disease (LAD), Lou Gehrig's disease, lupoid hepatitis, lupus erythematosus, Majeed syndrome, Meniere's disease, microscopic polyangiitis, Miller-Fisher syndrome, mixed connective tissue disease, morphea, Mucha-Habermann disease, multiple sclerosis, myasthenia gravis, myositis, neuropyelitis optica, neuromyotonia, ocular cicatricial pemphigoid, opsoclonus myoclonus syndrome, ord thyroiditis, palindromic rheumatism, paraneoplastic cerebellar degeneration, paroxysmal nocturnal hemoglobinuria (PNH), Parry Romberg syndrome, Parsonnage-Turner syndrome, pars planitis, pemphigus, pemphigus vulgaris, permicious anemia, perivenous encephalomyelitis, POEMS syndrome, polyarteritis nodosa, polymyalgia rheumatica, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restless leg syndrome, retroperitoneal fibrosis, rheumatoid arthritis, rheumatoid fever, sarcoidosis, Schmidt syndrome, Schnitzler syndrome, scleritis, scleroderma, Sjogren's syndrome, spondylarthropathy, Still's disease, stiff person syndrome, subacute bacterial endocarditis, Susac's syndrome, Sweet's syndrome, Sydenham chorea, sympathetic ophthalmia, Takayasu's arteritis, temporal arteritis, Tolosa-Hunt syndrome, transverse myelitis, ulcerative colitis, undifferentiated connective tissue disease, undifferentiated spondylarthropathy, vasculitis, vitiligo or Wegener's granulomatosis.

In some embodiments, the cell or the population of cells comprises a hematopoietic stem cell (HSC) or a derivative thereof.

In some embodiments, the condition or disease is associated with Parkinson's disease, Huntington disease, multiple sclerosis, a neurodegenerative disease or condition, attention deficit hyperactivity disorder (ADHD), Tourette Syndrome (TS), schizophrenia, psychosis, depression, a neuropsychiatric disorder stroke, or amyotrophic lateral sclerosis (ALS), or wherein the disease or condition is Parkinson's disease, Huntington disease, multiple sclerosis, a neurodegenerative disease or condition, attention deficit hyperactivity disorder (ADHD), Tourette Syndrome (TS), schizophrenia, psychosis, depression, a neuropsychiatric disorder stroke, or amyotrophic lateral sclerosis (ALS).

In some embodiments, the cell or the population of cells comprise a neural cell or a glial cell.

In some embodiments, the cell or the population of cells are expanded and cryopreserved prior to administration.

In some embodiments, the method comprises intravenous injection, intramuscular injection, intravascular injection, or transplantation of the cell, the population of cells, or the composition.

In some embodiments, transplantation comprises intravascular injection or intramuscular injection.

In some embodiments, the method further comprises administering one or more immunosuppressive agents to the subject.

In some embodiments, the subject has been administered one or more immunosuppressive agents.

In some embodiments, the method does not comprise administering one or more immunosuppressive agents to the subject.

In some embodiments, the subject is not immunosuppressed i.e., has not been administered one or more immunosuppressive agents.

In some embodiments, the one or more immunosuppressive agents are a small molecule or an antibody.

In some embodiments, the one or more immunosuppressive agents are selected from the group consisting of cyclosporine, azathioprine, mycophenolic acid, mycophenolate mofetil, a corticosteroids, prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin (thymosin-α), an immunomodulatory agent, and an immunosuppressive antibody.

In some embodiments, the one or more immunosuppressive agents comprise cyclosporine.

In some embodiments, the one or more immunosuppressive agents comprise mycophenolate mofetil.

In some embodiments, the one or more immunosuppressive agents comprise a corticosteroid.

In some embodiments, the one or more immunosuppressive agents comprise cyclophosphamide.

In some embodiments, the one or more immunosuppressive agents comprise rapamycin.

In some embodiments, the one or more immunosuppressive agents comprise tacrolimus (FK-506).

In some embodiments, the one or more immunosuppressive agents comprise anti-thymocyte globulin.

In some embodiments, the one or more immunosuppressive agents are one or more immunomodulatory agents.

In some embodiments, the one or more immunomodulatory agents are a small molecule or an antibody.

In some embodiments, the antibody binds to one or more receptors or ligands selected from the group consisting of p75 of the IL-2 receptor, MHC, CD2, CD3, CD4, CD7, CD28, B7, CD40, CD45, IFN-gamma, TNF-alpha, IL-4, IL-5, IL-6R, IL-6, IGF, IGFR1, IL-7, IL-8, IL-10, CD11a, CD58, and antibodies binding to any of their ligands.

In some embodiments, the one or more immunosuppressive agents are or have been administered to the subject prior to administration of the cell, the population of cells, or the composition.

In some embodiments, the one or more immunosuppressive agents are or have been administered to the subject at least 1, 2, 3, 4, 5, 6, 7, 8, 9, 10, 11, 12, 13, or 14 days prior to administration of the cell, the population of cells, or the composition.

In some embodiments, the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks or more prior to administration of the cell, the population of cells, or the composition.

In some embodiments, the one or more immunosuppressive agents are or have been administered to the subject after administration of the cell, the population of cells, or the composition.

In some embodiments, the one or more immunosuppressive agents are or have been administered to the subject at least 1, 2, 3, 4, 5, 6, 7, 8, 9, 10, 11, 12, 13, or 14 days after administration of the cell, the population of cells, or the composition.

In some embodiments, the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks, or more, after administration of the cell, the population of cells, or the composition.

In some embodiments, the one or more immunosuppressive agents are or have been administered to the subject on the same day as the first administration of the cell, the population of cells, or the composition.

In some embodiments, the one or more immunosuppressive agents are or have been administered to the subject after administration of a first and/or second administration of the cell, the population of cells, or the composition.

In some embodiments, the one or more immunosuppressive agents are or have been administered to the subject prior to administration of a first and/or second administration of the cell, the population of cells, or the composition.

In some embodiments, the one or more immunosuppressive agents are or have been administered to the subject at least 1, 2, 3, 4, 5, 6, 7, 8, 9, 10, 11, 12, 13, or 14 days prior to administration of a first and/or second administration of the cell, the population of cells, or the composition.

In some embodiments, the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks or more prior to administration of a first and/or second administration of the cell, the population of cells, or the composition.

In some embodiments, the one or more immunosuppressive agents are or have been administered to the subject at least 1, 2, 3, 4, 5, 6, 7, 8, 9, 10, 11, 12, 13, or 14 days after administration of a first and/or second administration of the cell, the population of cells, or the composition.

In some embodiments, the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks, or more, after administration of a first and/or second administration of the cell, the population of cells, or the composition.

In some embodiments, the one or more immunosuppressive agents are administered at a lower dosage as compared to the dosage administered to reduce immune rejection of a cell that does not comprise the one or more modifications of the cell or the population of cells.

In some embodiments, the method further comprises activating the safety switch to induce controlled cell death after the administration of the cell, the population of cells, or the composition to the subject.

In some embodiments, the suicide gene or the suicide switch is activated to induce controlled cell death after the administration of the one or more immunosuppressive agents to the subject.

In some embodiments, the suicide gene or the suicide switch is activated to induce controlled cell death prior to the administration of the one or more immunosuppressive agents to the subject.

In some embodiments, the safety switch is activated to induce controlled cell death in the event of cytotoxicity or other negative consequences to the subject.

In some embodiments, the safety switch is an “uncloaking” system wherein upon activation, cells downregulate expression of immunosuppressive factors and/or upregulate expression of immune signaling molecules thereby marking the cell for elimination by the host immune system.

In some embodiments, the method comprises administering an agent that allows for depletion of the cell, the population of cells, or the composition.

In some embodiments, the agent that allows for depletion of the cell is an antibody that recognizes a protein expressed on the cell surface.

In some embodiments, the antibody is selected from the group consisting of an antibody that recognizes CCR4, CD16, CD19, CD20, CD30, EGFR, GD2, HER1, HER2, MUC1, PSMA, and RQR8.

In some embodiments, the antibody is selected from the group consisting of mogamulizumab, AFM13, MOR208, obinutuzumab, ublituximab, ocaratuzumab, rituximab, rituximab-Rllb, tomuzotuximab, RO5083945 (GA201), cetuximab, Hul4.18K322A, Hul4.18-IL2, Hu3F8, dinituximab, c.60C3-Rllc, and biosimilars thereof.

In some embodiments, the method comprises administering an agent that recognizes the one or more tolerogenic factors or the one or more additional tolerogenic factors on the cell surface.

In some embodiments, the method further comprises administering one or more additional therapeutic agents to the subject.

In some embodiments, the subject has been administered one or more additional therapeutic agents.

In some embodiments, the method further comprises monitoring the therapeutic efficacy of the method.

In some embodiments, the method further comprises monitoring the prophylactic efficacy of the method.

In some embodiments, the method is repeated until a desired suppression of one or more disease symptoms occurs.

Computer-implemented methods and related systems and devices are also provided herein.

In one aspect, the present disclosure provides a computer-implemented method for profiling the donor capability of a cell or population of cells for cell therapy, the method comprising:
receiving, by one or more processors, data corresponding to a cell or a population of cells evaluating, by the one or more processors, the received data for predicted function; and
profiling. by the one or more processors, a suitability of the cell or a suitability of the population of cells for donor capability as a population of cells for cell therapy.

In some embodiments, the evaluating is performed according to any evaluation method as described herein.

The profiling may be performed according to any identifying method as described anywhere herein. The cells or the population of cells may be as described anywhere herein.

In one aspect, the present disclosure provides a computer-implemented method wherein at least one processor performs the method as described anywhere herein.

In one aspect, the present disclosure provides a computer program comprising instructions which, when the program is executed by a computer comprising at least one processor, cause the computer to carry out the method as described herein.

In one aspect, the present disclosure provides a computer-readable medium comprising instructions which, when executed by a computer comprising at least one processor, cause the computer to carry out the method as described herein.

In one aspect, the present disclosure a provides a data processing device comprising at least one processor and memory, the memory comprising instructions for carrying out the method as described herein.

In some embodiments, the data processing device is a cloud service.

In one aspect, the present disclosure provides a system comprising a local data processing device comprising at least one processor and memory, and a remote data processing device comprising at least one processor and memory, wherein: the memory of the local data processing device comprising instructions to measure data corresponding to a cell or a population of cells and transmit the data to the remote data processing device. In some embodiments, the memory of the remote data processing device comprising instructions to perform the method as described herein.

In one aspect, the present disclosure provides a method comprising: classifying a donor capability for cell therapy of a cell or a population of cells of a sample, comprising: providing a cell or a population of cells obtained from the sample; assaying a cell function parameter of the cell or the population of cells from the sample; receiving, at one or more processors, test data for the sample, wherein the test data comprises an assay readout for the cell or the population of cells from the sample; inputting, using the one or more processors, the test data into a cell function model, wherein the cell function model is configured to classify the sample, based on the test data, as good donor capability-like, or not good donor capability-like; and classifying, by the one or more processors using the cell function model, the sample as good donor capability-like, or not good donor capability-like.

In one aspect, the present disclosure provides a method comprising: scoring a donor capability for cell therapy of a cell or a population of cells of a sample, comprising: providing a cell or a population of cells obtained from the sample; assaying a cell function parameter of the cell or the population of cells from the sample; receiving, at one or more processors, test data for the sample, wherein the test data comprises an assay readout for the cell or the population of cells from the sample; inputting, using the one or more processors, the test data into a cell function model, wherein the cell function model is configured to score the sample, based on the test data, with a number corresponding to donor capability; and scoring, by the one or more processors using the cell function model, the sample with a number corresponding to donor capability.

In some embodiments, the method as described herein, wherein the model is a mathematical model, a statistical model, a grey box model, a blockmodel, a predictive model, and a deterministic model, or a machine learning model. In some embodiments, the machine learning model comprising a module employing a regression-based model, a regularization-based model, an instance-based mode, a Bayesian-based model, a clustering-based model, an ensemble-based model, or a neural-network-based model.

In some embodiments, the method as described herein, wherein the cell function model is a machine-learning model trained using assay readout data comprising assay readouts from a plurality of cell samples and cell function data comprising cell function data from a plurality of cell samples.

In some embodiments, the method as described herein, wherein the assaying the cell function parameter is as described herein.

In one aspect, the present disclosure provides a method for classifying a donor capability of a cell or a population of cells for cell therapy, the method comprising: receiving, at one or more processors, test data comprising assay readouts for the cell or the population of cells; inputting, using the one or more processors, the test data into a cell function model, wherein the cell function model is configured to classify the cell or the population of cells based on the test data as good donor capability-like, or not good donor capability-like; and classifying, using the one or more processors and the cell function model, the cell or the population of cells as good donor capability-like, or not good donor capability-like.

In one aspect, the present disclosure provides a method for scoring a donor capability of a cell or a population of cells for cell therapy, the method comprising: receiving, at one or more processors, test data comprising assay readouts for the cell or the population of cells; inputting, using the one or more processors, the test data into a cell function model, wherein the cell function model is configured to score the cell or the population of cells based on the test data with a donor capability value; and scoring, using the one or more processors and the cell function model, the cell or the population of cells with a donor capability value.

In some embodiments, the method as described herein, wherein the model is a mathematical model, a statistical model, a grey box model, a blockmodel, a predictive model, and a deterministic model, or a machine learning model. In some embodiments, the machine learning model comprising a module employing a regression-based model, a regularization-based model, an instance-based mode, a Bayesian-based model, a clustering-based model, an ensemble-based model, or a neural-network-based model.

In some embodiments, the method as described herein, wherein the cell function model is a machine-learning model trained using assay readout data comprising assay readouts from a plurality of cell samples and cell function data comprising cell function data from a plurality of cell samples

In some embodiments, the method as described herein, further comprising training the cell function model using the assay readout data and the cell function data.

In one aspect, the present disclosure provides a system, comprising: one or more processors; a memory; and one or more programs, wherein the one or more programs are stored in the memory and configured to be executed by the one or more processors, the one or more programs including instructions for implementing a method, comprising: receiving, at the one or more processors, test data comprising assay readout data for a sample of a cell or a population of cells from a subject; inputting, using the one or more processors, the test data into a cell function model, wherein the cell function model is configured to classify the sample, based on the test data, as good donor capability-like, or not good donor capability-like; and classifying, using the one or more processors and the cell function model, the sample as good donor capability-like, or not good donor capability-like.

In one aspect, the present disclosure provides a system, comprising: one or more processors; a memory; and one or more programs, wherein the one or more programs are stored in the memory and configured to be executed by the one or more processors, the one or more programs including instructions for implementing a method, comprising: receiving, at the one or more processors, test data comprising assay readout data for a sample of a cell or a population of cells from a subject; inputting, using the one or more processors, the test data into a cell function model, wherein the cell function model is configured to score the sample, based on the test data, with a number value corresponding to donor capability; and scoring, using the one or more processors and the cell function model, the sample with a number corresponding to donor capability.

In some embodiments, the system as described herein, wherein the model is a mathematical model, a statistical model, a grey box model, a blockmodel, a predictive model, and a deterministic model, or a machine learning model. In some embodiments, the machine learning model comprising a module employing a regression-based model, a regularization-based model, an instance-based mode, a Bayesian-based model, a clustering-based model, an ensemble-based model, or a neural-network-based model.

In some embodiments, the system as described herein, wherein the cell function model is a machine-learning model trained using assay readout data comprising assay readouts from a plurality of cell samples and cell function data comprising cell function data from a plurality of cell samples.

In some embodiments, the system as described herein, wherein the one or more programs further include instructions for training the cell function model using the assay readout data and the cell function data.

In one aspect, the present disclosure provides a non-transitory computer-readable storage medium storing one or more programs, the one or more programs comprising instructions, which when executed by one or more processors of an electronic device, cause the electronic device to implement a method, comprising: receiving, at the one or more processors, test data comprising at least one assay readout for the cell or the population of cells; inputting, using the one or more processors, the test data into a cell function model, wherein the cell function model is configured to classify the sample, based on the test data, as good donor capability-like, or not good donor capability-like; and classifying, using the one or more processors and the cell function model, the sample as good donor capability-like, or not good donor capability-like.

In one aspect, the present disclosure provides a non-transitory computer-readable storage medium storing one or more programs, the one or more programs comprising instructions, which when executed by one or more processors of an electronic device, cause the electronic device to implement a method, comprising: receiving, at the one or more processors, test data comprising at least one assay readout for the cell or the population of cells; inputting, using the one or more processors, the test data into a cell function model, wherein the cell function model is configured to score the sample, based on the test data, with a number corresponding to donor capability; and scoring, using the one or more processors and the cell function model, the sample with a number corresponding to donor capability.

In some embodiments, the non-transitory computer-readable storage medium as described herein, wherein the model is a mathematical model, a statistical model, a grey box model, a blockmodel, a predictive model, and a deterministic model, or a machine learning model. In some embodiments, the machine learning model comprising a module employing a regression-based model, a regularization-based model, an instance-based mode, a Bayesian-based model, a clustering-based model, an ensemble-based model, or a neural-network-based model.

In some embodiments, the non-transitory computer-readable storage medium as described herein, wherein the cell function model is a machine-learning model trained using assay readout data comprising assay readouts from a plurality of cell samples and cell function data comprising cell function data from a plurality of cell samples.

In some embodiments, the non-transitory computer-readable storage medium as described herein, wherein the one or more programs further include instructions, which when executed by one or more processors of an electronic device, cause the electronic device to train the cell function model using the assay readout data and the cell function data.

In one aspect, the present disclosure provides a method comprising: classifying a donor capability for cell therapy for a sample from a subject, comprising: providing a cell or a population of cells obtained from the sample from a subject; assaying a cell function parameter, wherein the assaying provides an assay readout as test data; receiving, at one or more processors, test data for the sample, wherein the test data comprises the assay readout; inputting, using the one or more processors, the test data into a model, wherein the first cell samples are different from the second cell samples, and wherein the model is configured to classify the sample, based on the test data, as first donor capability-like, or second donor capability-like; and classifying, by the one or more processors using the model, the sample as first donor capability-like, or second donor capability-like.

In one aspect, the present disclosure provides a method comprising: scoring a donor capability for cell therapy for a sample from a subject, comprising: providing a cell or a population of cells obtained from the sample from a subject; assaying a cell function parameter, wherein the assaying provides an assay readout as test data; receiving, at one or more processors, test data for the sample, wherein the test data comprises the assay readout; inputting, using the one or more processors, the test data into a model, wherein the first cell samples are different from the second cell samples, and wherein the model is configured to score the sample, based on the test data, with a number corresponding donor capability; and scoring, by the one or more processors using the model, the sample with a number corresponding donor capability.

In some embodiments, the method as described herein, wherein the model is a mathematical model, a statistical model, a grey box model, a blockmodel, a predictive model, and a deterministic model, or a machine learning model. In some embodiments, the machine learning model comprising a module employing a regression-based model, a regularization-based model, an instance-based mode, a Bayesian-based model, a clustering-based model, an ensemble-based model, or a neural-network-based model.

In some embodiments, the method as described herein, wherein the model is a machine-learning model trained using a first assay readout data comprising a first assay readout data from a plurality of first cell samples and a second assay readout data comprising second assay readout data from a plurality of second cell samples.

In some embodiments, the method as described herein, wherein the assaying the cell function parameter is as described herein.

In one aspect, the present disclosure provides a method, comprising: receiving, at one or more processors, test data for a sample, wherein the test data comprises an assay readout for a cell or a population of cells of the sample; inputting, using the one or more processors, the test data into a model, wherein the first cell samples are different from the second cell samples, and wherein the model is configured to classify the sample, based on the test data, as first donor capability-like, or second donor capability-like; and classifying, by the one or more processors using the model, the sample as first donor capability-like, or a second donor capability-like.

In one aspect, the present disclosure provides a method, comprising: receiving, at one or more processors, test data for a sample, wherein the test data comprises an assay readout for a cell or a population of cells of the sample; inputting, using the one or more processors, the test data into a model, wherein the first cell samples are different from the second cell samples, and wherein the model is configured to score the sample, based on the test data, with a number corresponding to donor capability; and scoring, by the one or more processors using the model, the sample with a number corresponding to donor capability.

In some embodiments, the method as described herein, wherein the model is a mathematical model, a statistical model, a grey box model, a blockmodel, a predictive model, and a deterministic model, or a machine learning model. In some embodiments, the machine learning model comprising a module employing a regression-based model, a regularization-based model, an instance-based mode, a Bayesian-based model, a clustering-based model, an ensemble-based model, or a neural-network-based model.

In some embodiments, the method as described herein, wherein the model is a machine-learning model trained using a first assay readout data comprising a first assay readout data from a plurality of first cell samples and a second assay readout data comprising second assay readout data from a plurality of second cell samples.

In some embodiments, the method, system or non-transitory computer-readable storage medium as described herein, wherein: the model is trained to classify the cell or population of cells, or the sample, based on the test data, as ambiguous in addition to first donor capability-like or second donor capability-like; and/or the model is trained to classify the cell or population of cells, or the sample, based on the test data, as ambiguous in addition to as first donor capability-like or second donor capability-like.

In some embodiments, the method, system or non-transitory computer-readable storage medium as described herein, wherein the model or cell function model is trained to classify the cell or population of cells and/or the sample as one or more additional classification(s) or one or more alternative classification(s).

In one aspect, the present disclosure provides a method of evaluating a cell or a population of cells for predicted function, the method comprising: receiving, by one or more processors, input data corresponding to the cell or the population of cells; and determining (e.g., inferring), by the one or more processors, a predicted function of the cell or the population of cells using the input data and reference data corresponding to reference cells and/or populations of reference cells (e.g., by comparing the input data and the reference data).

In one aspect, the present disclosure provides a method of predicting in vivo function of a cell or a population of cells, the method comprising: receiving, by one or more processors, input data corresponding to the cell or the population of cells; and determining (e.g., inferring), by the one or more processors, a predicted in vivo function of the cell or the population of cells using the input data and reference data corresponding to reference cells and/or populations of reference cells [e.g., by comparing the input data and the reference data].

In one aspect, the present disclosure provides a method of training a model to predict function of a cell or a population of cells, the method comprising training, by one or more processors, an algorithm (e.g., in a machine learning module) using reference data corresponding to reference cells and/or populations of reference cells (e.g., obtained using a method as described herein).

In one aspect, the present disclosure provides a method of training a model to predict in vivo cell function of a cell or a population of cells, the method comprising training, by one or more processors, an algorithm (e.g., in a machine learning module) using reference data corresponding to reference cells and/or populations of reference cells.

In one aspect, the present disclosure provides a method of determining one or more cell characteristics (e.g., function(s) and/or parameter(s)) (e.g., cell type (or subtype) specific characteristics) indicative of cell(s) being suitable as donor cell(s) for a cell therapy, the method comprising comparing, by one or more processors, reference data generated from reference cells and/or populations of reference cells [e.g., using an algorithm (e.g., in a machine learning module)].

In some embodiments, (i) the reference data comprises one or more quantitative assay readouts from reference cells and/or the populations of reference cells (e.g., determined using a (e.g., computer-implemented) method as described herein), one or more qualitative assessments of one or more quantitative assay readouts from reference cells and/or the populations of reference cells (e.g., determined using a (e.g., computer-implemented) method as described herein), or a combination thereof, (ii) the input data comprises one or more quantitative assay readouts from the cell or the population of cells (e.g., determined using a (e.g., computer-implemented) method as described herein), one or more qualitative assessments of one or more quantitative assay readouts from the cell or the population of cells (e.g., determined using a (e.g., computer-implemented) method as described herein), or a combination thereof, or (iii) both (i) and (ii).

In some embodiments, the input data and/or the reference data comprises data corresponding to age of subject(s)(e.g., from which the cell or the population of cells is derived and/or from which the reference cells and/or the populations of reference cells are derived, respectively), health history [e.g., health status (e.g., current health status)] of the subject(s), sex of the subject(s), or a combination thereof.

In some embodiments, the reference data comprises (e.g., further comprises) clinical output data, in vitro experimental data, in vivo experimental data, or a combination thereof (e.g., determined using a (e.g., computer-implemented) method as described herein).

In some embodiments, the input data and/or the reference data comprises one or more quantitative assay readouts from one or more assays and the one or more assays comprise an in vitro assay, an in vivo assay, an immune assay, a cell activity assay, a cell avidity assay, a cell proliferation assay, a cell cytotoxicity assay, a cellular stress assay, a tumor challenge assay, an expression assay, a cytokine production assay, transcriptomic profiling assay, a proteomic profiling assay, a genomic profiling assay, a genomic stability assay, an epigenetic profiling assay, a cell developmental potential profiling assay, a cell subtyping assay, a cell receptor profiling assay or a combination thereof.

In some embodiments, the one or more quantitative assay readouts comprises a cell functionality score, a cell polyfunctionality index, a cell multifunctionality index, an in vivo efficacy score, an in vivo activity score, an in vivo response score, an in vitro efficacy score, an in vitro activity score, an in vitro response score, an immune efficacy score, an immune activity score, an immune response score, a cell activity score, a cell activity response score, a cell specificity score, a cell sensitivity score, a cell avidity score, a cell proliferation score, a cell proliferative index, a cell cytotoxicity score, a cell cytotoxicity response score, a cell stress score, a cell stress response score, a tumor challenge efficacy score, a tumor challenge activity score, a tumor challenge response score, a tumor challenge specificity score, a tumor challenge sensitivity score, an expression profile, an expression signature, an expression signal, an expression score, a bulk cytokine or chemokine production profile, a bulk cytokine or chemokine production signature, a bulk cytokine or chemokine production profile, a bulk cytokine or chemokine production signal, a bulk cytokine or chemokine production score, a single cell cytokine or chemokine production profile, a single cell cytokine or chemokine production signature, a single cell cytokine or chemokine production profile, a single cell cytokine or chemokine production signal, a single cell cytokine or chemokine production score, a transcriptomic profile, a transcriptomic signature, a transcriptomic signature, a transcriptomic score, a pathway profile, a pathway signature, a pathway signal, a pathway score, a proteomic profile, a proteomic signature, a proteomic signal, a proteomic score, a genomic profile, a genomic signature, a genomic signal, a genomic score, a genomic stability profile, a genomic stability signature, a genomic stability signal, a genomic stability score, an epigenetic profile, an epigenetic signature, an epigenetic signal, an epigenetic score, a cell developmental potential assessment, a cell developmental potential profile, a cell developmental potential signature, a cell developmental potential score, a cell subtyping profile, a cell subtyping signature, a cell subtyping score, a cell receptor profile, a cell receptor signature, a cell receptor signal, a cell receptor score, immune cell identity profile, immune cell subtyping profile, cell subtype ratio profile, cell proliferation profile, cell proliferation score, HLA typing profile, transcriptomic profile or a combination thereof.

In some embodiments, the input data and/or the reference data comprises data corresponding to (i) one or more assay readouts, optionally cell activation, cell polyfunctionality, cell multifunctionality, cell cytotoxicity, cell growth rate, one or more characteristics associated with particular cell type, cellular activity associated with the cell or the population of cells, cell cytokine production, immune cell identity, immune cell subtyping, cell subtype ratio, cell proliferation, HLA typing, a transcriptome, or a combination thereof, and/or (ii) data relating to function (e.g., predicted function), optionally durability of cell growth, durability of cell response, cellular ability to elicit adaptive and innate immune responses, or a combination thereof; and optionally cell safety attributes; and/or cell impurity level(s). In some embodiments, the input data and/or the reference data comprises one or more assay readouts and/or data relating to function (e.g., predicted function) from one or more assessments of the cells or the populations of cells or the reference cells or the reference populations of cells, optionally at one or more time points.

In some embodiments, the one or more qualitative assessments of one or more quantitative assay readouts are qualitative categorizations (e.g., poor, good, usable, or exceptional) (e.g., determined using a (e.g., computer-implemented) method as described herein).

In some embodiments, the reference data comprises persistence data, engraftment data, durability (e.g., of cell response) data, potency data, hypoimmunogenicity data, or a combination thereof (e.g., determined using a (e.g., computer-implemented) method as described herein).

In some embodiments, the reference cells and/or the populations of reference cells comprise suitable donor cells and/or unsuitable donor cells (e.g., determined using a (e.g., computer-implemented) method as described herein).

In some embodiments, cell or the population of cells comprises a cell as described herein.

In some embodiments, the reference cells and/or populations of reference cells do not have any modification.

In some embodiments, the reference cells and/or populations of reference cells have been modified (e.g., in accordance with any embodiments as described herein).

In some embodiments, (i) the input data comprise data corresponding to at least one cell parameter and/or at least one function of the cell or the population of cells, (ii) the reference data comprise data corresponding to at least one cell parameter and/or at least one function of the reference cells and/or the populations of reference cells, or (iii) both (i) and (ii).

In some embodiments: (i) the input data comprises one or more quantitative assay readouts from the cell or the population of cells and the one or more quantitative assay readouts for the cell or the population of cells comprises one or more single values (e.g., one or more numerical values), one or more complex values (e.g., comprising a plurality of values) (e.g., comprising a range of values) [e.g., comprising multiple separate numerical values (e.g., pixel data from one or more images and/or flow plot data (e.g., from a gel))], one or more qualitative or semi-quantitative values [e.g., comprising one or more non-numerical values (e.g., a qualitative scale)], or a combination thereof (e.g., determined using a (e.g., computer-implemented) method as described herein), (ii) the reference data comprises one or more quantitative assay readouts from the reference cells and/or the populations of reference cells and the one or more quantitative assay readouts for the reference cells and/or the populations of reference cells comprises one or more single values (e.g., one or more numerical values), one or more complex values (e.g., comprising a plurality of values) (e.g., comprising a range of values) [e.g., comprising multiple separate numerical values (e.g., pixel data from one or more images and/or flow plot data (e.g., from a gel))], one or more qualitative or semi-quantitative values [e.g., comprising one or more non-numerical values (e.g., a qualitative scale)], or a combination thereof (e.g., determined using a (e.g., computer-implemented) method as described herein), or (iii) both (i) and (ii).

In some embodiments, the one or more quantitative assay readouts comprises the complex values and/or the qualitative or semi-quantitative values and the complex values and/or the qualitative or semi-quantitative values comprises pixel information from one or more images (e.g., wherein the one or more quantitative assay readouts comprises one or more images from one or more assays), flow plot data (e.g., wherein the one or more quantitative assay readouts comprises one or more images from one or more assays), or a combination thereof (e.g., determined using a (e.g., computer-implemented) method as described herein).

In some embodiments, the input data and/or the reference data comprises one or more single values that have been converted from one or more complex values and/or qualitative or semi-quantitative values of one or more quantitative assay readouts (e.g., from the cell or the population of cells and/or from the reference cells and/or the populations of reference cells) (e.g., prior to determining the predicted function) [e.g., using a predetermined conversion scheme (e.g., scale) (e.g., by combining (e.g., concatenating) and/or statistically processing (e.g., averaging), by the one or more processors, the values)].

In some embodiments, the one or more quantitative assay readouts (e.g., from the cell or the population of cells and/or from the reference cells and/or the populations of reference cells) comprise the complex values and/or the qualitative or semi-quantitative values and the method comprises converting, by the one or more processors, the complex values and/or the qualitative or semi-quantitative values to a single value for each of the one or more quantitative assay readouts (e.g., prior to determining the predicted function) [e.g., using a predetermined conversion scheme (e.g., scale)(e.g., by combining (e.g., concatenating) and/or statistically processing (e.g., averaging), by the one or more processors, the values)].

In some embodiments, determining the predicted function comprises comparing, by the one or more processors, one or more single values for one or more quantitative assay readouts for the cell or the population of cells and one or more single values for one or more quantitative assay readouts for the reference cells and/or the populations of reference cells (e.g., after converting, by the one or more processors, to the single value(s) from one or more complex values and/or one or more qualitative and/or semi-quantitative values).

In some embodiments of a method described herein, the method further comprising converting, by the one or more processors, one or more quantitative assay readouts into a suitable format for performing the determining (e.g., for input into a machine learning module) [e.g., suitable data format and/or size (e.g., string and/or number format and/or size)].

In some embodiments, determining the predicted function comprises using one or more weights to weight the input data and/or the reference data (e.g., relative to each other and/or to differently weight different portions of the input data and/or to differently weight different portions of the reference data) (e.g., used in a loss function used in a machine learning module) [e.g., that have been determined by a trained algorithm (e.g., in a machine learning module)].

In some embodiments of a method described herein, the method further comprising determining, by the one or more processors, the one or more weights.

In some embodiments, the one or more weights are for weighting one or more quantitative assay readouts comprised in the input data and/or the reference data.

In some embodiments, determining the one or more weights comprises receiving, by the one or more processors, user input corresponding to the one or more weights (e.g., via one or more graphical user interfaces (GUIs)).

In some embodiments, the user input comprises one or more of the one or more weights and/or one or more cell characteristics (e.g., function(s) and/or parameter(s)) to weight.

In some embodiments, determining the one or more weights comprises determining the one or more weights using a trained algorithm or training an algorithm (e.g., in a machine learning module).

In some embodiments, the one or more weights correspond to (e.g., wherein the user input represents):
(i) one or more cell characteristics to consider or not consider (e.g., one or more quantitative assay readouts to consider or not consider),
(ii) a preference of one or more cell characteristics over one or more other cell characteristics [e.g., a ranked list of two or more cell characteristics (e.g., a ranked list of some but not all or all available cell characteristics to choose from)] (e.g., a preference of one or more quantitative assay readouts over one or more other assay readouts),
(iii) a selection of one or more subsets of the input data and/or the reference data to weight more heavily or less heavily (e.g., comprising one or more desired weights [e.g., that is/are then fixed or that serves as an initial value that is/are then modified (e.g., through iteration in a machine learning module)], or
(iv) a combination thereof.

In some embodiments of a method described herein, the method further comprising comparing, by the one or more processors, the input data and (e.g., to) the reference data (e.g., using a machine learning module).

In some embodiments, comparing the input data and the reference data comprises performing, by the one or more processors, a multiparametric comparison between the input data and the reference data.

In some embodiments of a method described herein, the method further comprising comparing (e.g., optimizing), by the one or more processors, two or more, three or more, four or more, five or more, six or more, seven or more, eight or more, nine or more, or ten or more parameters.

In some embodiments, the multiparametric comparison is a biomarker.

In some embodiments, the predicted function comprises one or more of functions selected from the group consisting of durability of cell growth, durability of cell response, and cellular ability to elicit adaptive and innate immune responses.

In some embodiments of a method described herein, the method further comprising determining (e.g., classifying and/or inferring), by the one or more processors, (e.g., wherein a machine learning module that determines the predicted function outputs) a categorization of the cell or the population of cells that is indicative of the predicted function of the cell or the population of cells.

In some embodiments, the predicted function comprises a categorization of the cell or the population of cells.

In some embodiments, the categorization is a qualitative categorization (e.g., that uses a qualitative scale comprising one or more of: ‘poor,’ ‘usable,’ ‘good,’ and ‘exceptional’).

In some embodiments, the categorization uses a quantitative scale (e.g., provides a numerical value on a predetermined scale).

In some embodiments, the categorization is indicative of a suitability of the cell or the population of cells for use as donor cell(s) in a cell therapy.

In some embodiments of a method described herein, the method further comprising: determining (e.g., classifying and/or inferring), by the one or more processors, suitability of the cell or the population of cells for use as donor cell(s) in a cell therapy (e.g., wherein the predicted function is indicative of the suitability) (e.g., wherein the suitability is determined using the predicted function) (e.g., using the input data and the reference data in a (e.g., computer-implemented) method to determine suitability as described herein).

In some embodiments, determining the suitability comprises determining (e.g., classifying and/or inferring), by the one or more processors, that the cell or the population of cells is/are suitable for use as donor cell(s) (e.g., by a (e.g., computer-implemented) method as described herein).

In some embodiments, the input data comprises data corresponding to one or more cell characteristics (e.g., function(s) and/or parameter(s)) and the method comprises determining (e.g., classifying and/or inferring), by the one or more processors that the cell or the population of cells is/are suitable for use as donor cell(s) due, in part, to presence and/or absence of one or more traits from the one or more cell characteristics (e.g., by a (e.g., computer-implemented) method as described herein).

In some embodiments, the one or more cell characteristics correspond to age of subject from which the cell or the population of cells is derived, health history [e.g., health status (e.g., current health status)] of the subject, sex of the subject, or a combination thereof [e.g., and the one or more traits correspond to an acceptable and/or desirable (e.g., preferred) age or age range, acceptable and/or desirable (e.g., preferred) sex, presence of one or more acceptable and/or desirable (e.g., preferred) health traits, absence of one or more unacceptable and/or undesirable (e.g., non-preferred) health traits, or a combination thereof].

In some embodiments of a method described herein, the method further comprising determining (e.g., classifying and/or inferring), by the one or more processors, that the cell or the population of cells is suitable due to the presence and/or absence of the one or more traits when the cell or the population of cells would otherwise be less suitable or unsuitable without and/or with having the one or more traits, respectively (e.g., by a (e.g., computer-implemented) method as described herein).

In some embodiments, determining the suitability comprises using one or more weights to weight the input data and/or the reference data (e.g., relative to each other and/or to differently weight different portions of the input data and/or to differently weight different portions of the reference data)(e.g., used in a loss function used in a machine learning module).

In some embodiments of a method described herein, the method further comprising determining, by the one or more processors, one or more weights for use in determining the suitability (e.g., used in a loss function used in a machine learning module).

In some embodiments, the one or more weights correspond to one or more desirable cell characteristics and/or the one or more weights have been selected (e.g., by user input) to more heavily weight one or more desirable cell characteristics than one or more other cell characteristics (e.g., wherein the one or more desirable cell characteristics are or have been determined, by the one or more processors, using reference data).

In some embodiments, the one or more weights are for weighting one or more quantitative assay readouts comprised in the input data and/or the reference data.

In some embodiments, determining the one or more weights comprises receiving, by the one or more processors, user input corresponding to the one or more weights (e.g., via one or more graphical user interfaces (GUIs)).

In some embodiments, the user input comprises one or more of the one or more weights and/or one or more cell characteristics to weight.

In some embodiments, determining the one or more weights comprises (i) determining the one or more weights using a trained algorithm or (ii) training an algorithm (e.g., in a machine learning module).

In some embodiments, the one or more weights correspond to (e.g., wherein the user input represents):
(i) one or more cell characteristics to consider or not consider (e.g., one or more quantitative assay readouts to consider or not consider),
(ii) a preference of one or more cell characteristics over one or more other cell characteristics [e.g., a ranked list of two or more cell characteristics (e.g., a ranked list of some but not all or all available cell characteristics to choose from)] (e.g., a preference of one or more quantitative assay readouts over one or more other assay readouts),
(iii) a selection of one or more subsets of the input data and/or the reference data to weight more heavily or less heavily (e.g., comprising one or more desired weights [e.g., that is/are then fixed or that serves as an initial value that is/are then modified (e.g., through iteration in a machine learning module)], or
(iv) a combination thereof.

In some embodiments, the method is performed using (e.g., wherein the predicted function of the cell or the population of cells is determined using) a machine learning module that (a) has been trained using the reference data and (b) receives the input data as an input [e.g., in addition to one or more other inputs (e.g., user-selected weight(s) to be applied (e.g., in a loss function))].

In some embodiments, the machine learning module employs a regression-based model (e.g., a logistic regression model), a regularization-based model (e.g., an elastic net model or a ridge regression model), an instance-based model (e.g., a support vector machine or a k-nearest neighbor model), a Bayesian-based model (e.g., a naïve-based model or a Gaussian naïve-based model), a clustering-based model (e.g., an expectation maximization model), an ensemble-based model (e.g., an adaptive boosting model, a random forest model, a bootstrap-aggregation model, or a gradient boosting machine model), or a neural-network-based model (e.g., a convolutional neural network, a recurrent neural network, autoencoder, a back propagation network, or a stochastic gradient descent network).

In one aspect, the present disclosure provides a system comprising one or more processors; a memory; one or more programs; and optionally a machine learning module (e.g., stored in the memory), wherein the one or more programs are stored in the memory and are executable by the one or more processors, the one or more programs including instructions for implementing a method as described anywhere herein.

In some embodiments of a system described herein, the method further comprising performing one or more assays and/or one or more qualitative assessments of one or more quantitative assay readouts (e.g., in a method as described herein) to generate the input data [e.g., that is then converted to a different size and/or format (e.g., a single value) (e.g., prior to being input into a machine learning module)].

In some embodiments, performing the one or more assays comprises obtaining the cell or the population of cells, assaying the cell or the population of cells for at least one cell parameter, and obtaining at least one assay readout.

In some embodiments, the one or more assays assay for 2 or more, 3 or more, four or more, five or more, six or more, seven or more, eight or more, nine or more, or at least ten cell parameters.

In one aspect, the present disclosure provides a method comprising making a cell therapy using a suitable cell or population of cells as donor cell(s) (e.g., in a method as described herein), wherein the cell or the population of cells have been determined to be suitable by a method (for example a computer-based method) as described herein. In one aspect, the present disclosure provides a method comprising administering a cell therapy to a subject (e.g., in a method as described herein), wherein the cell therapy comprises one or more donor cells that have been determined to be suitable by a method (for example a computer-based method) as described herein. In some embodiments of a method described herein, the method further comprising introducing into the cell or the population of cells one or more modifications (e.g., in accordance with any aspects and embodiments described herein).

In one aspect, the present disclosure provides a system for evaluating a cell or a population of cells for predicted function, the system comprising one or more processors; a memory; and one or more programs, wherein the one or more programs are stored in the memory and are executable by the one or more processors, the one or more programs comprising instructions for implementing a method comprising: receiving, by one or more processors, input data corresponding to the cell or the population of cells; and determining (e.g., inferring), by the one or more processors, a predicted function of the cell or the population of cells using the input data and reference data corresponding to reference cells and/or populations of reference cells (e.g., by comparing the input data and the reference data).

In one aspect, the present disclosure provides a system for predicting in vivo function of a cell or a population of cells, the system comprising one or more processors; a memory; and one or more programs, wherein the one or more programs are stored in the memory and are executable by the one or more processors, the one or more programs comprising instructions for implementing a method comprising: receiving, by one or more processors, input data corresponding to the cell or the population of cells; and determining (e.g., inferring), by the one or more processors, a predicted in vivo function of the cell or the population of cells using the input data and reference data corresponding to reference cells and/or populations of reference cells [e.g., by comparing the input data and the reference data].

In one aspect, the present disclosure provides a system for training a model to predict function of a cell or a population of cells, the system comprising one or more processors; a memory; and one or more programs, wherein the one or more programs are stored in the memory and are executable by the one or more processors, the one or more programs comprising instructions for implementing a method comprising training, by one or more processors, an algorithm (e.g., in a machine learning module) using reference data corresponding to reference cells and/or populations of reference cells (e.g., obtained using a method disclosed herein).

In one aspect, the present disclosure provides a system for training a model to predict in vivo cell function of a cell or a population of cells, the system comprising one or more processors; a memory; and one or more programs, wherein the one or more programs are stored in the memory and are executable by the one or more processors, the one or more programs comprising instructions for implementing a method comprising training, by one or more processors, an algorithm (e.g., in a machine learning module) using reference data corresponding to reference cells and/or populations of reference cells.

In one aspect, the present disclosure provides a system for determining one or more cell characteristics (e.g., function(s) and/or parameter(s)) (e.g., cell type (or subtype) specific characteristics) indicative of cell(s) being suitable as donor cell(s) for a cell therapy, the system comprising one or more processors; a memory; and one or more programs, wherein the one or more programs are stored in the memory and are executable by the one or more processors, the one or more programs comprising instructions for implementing a method comprising comparing, by one or more processors, reference data generated from reference cells and/or populations of reference cells [e.g., using an algorithm (e.g., in a machine learning module)].

In some embodiments, (i) the reference data comprises one or more quantitative assay readouts from reference cells and/or the populations of reference cells (e.g., determined using a (e.g., computer-implemented) method disclosed herein), one or more qualitative assessments of one or more quantitative assay readouts from reference cells and/or the populations of reference cells (e.g., determined using a (e.g., computer-implemented) method in accordance with any preceding embodiment), or a combination thereof, (ii) the input data comprises one or more quantitative assay readouts from the cell or the population of cells (e.g., determined using a (e.g., computer-implemented) method disclosed herein), one or more qualitative assessments of one or more quantitative assay readouts from the cell or the population of cells (e.g., determined using a (e.g., computer-implemented) method disclosed herein), or a combination thereof, or (iii) both (i) and (ii).

In some embodiments, the input data and/or the reference data comprises data corresponding to age of subject(s)(e.g., from which the cell or the population of cells is derived and/or from which the reference cells and/or the populations of reference cells are derived, respectively), health history [e.g., health status (e.g., current health status)] of the subject(s), sex of the subject(s), or a combination thereof.

In some embodiments, the reference data comprises (e.g., further comprises) clinical output data, in vitro experimental data, in vivo experimental data, or a combination thereof (e.g., determined using a (e.g., computer-implemented) method disclosed herein).

In some embodiments, the input data and/or the reference data comprises one or more quantitative assay readouts from one or more assays and the one or more assays comprise an in vitro assay, an in vivo assay, an immune assay, a cell activity assay, a cell avidity assay, a cell proliferation assay, a cell cytotoxicity assay, a cellular stress assay, a tumor challenge assay, an expression assay, a cytokine production assay, transcriptomic profiling assay, a proteomic profiling assay, a genomic profiling assay, a genomic stability assay, an epigenetic profiling assay, a cell developmental potential profiling assay, a cell subtyping assay, a cell receptor profiling assay or a combination thereof.

In some embodiments, the one or more quantitative assay readouts comprises a cell functionality score, a cell polyfunctionality index, a cell multifunctionality index, an in vivo efficacy score, an in vivo activity score, an in vivo response score, an in vitro efficacy score, an in vitro activity score, an in vitro response score, an immune efficacy score, an immune activity score, an immune response score, a cell activity score, a cell activity response score, a cell specificity score, a cell sensitivity score, a cell avidity score, a cell proliferation score, a cell proliferative index, a cell cytotoxicity score, a cell cytotoxicity response score, a cell stress score, a cell stress response score, a tumor challenge efficacy score, a tumor challenge activity score, a tumor challenge response score, a tumor challenge specificity score, a tumor challenge sensitivity score, an expression profile, an expression signature, an expression signal, an expression score, a bulk cytokine or chemokine production profile, a bulk cytokine or chemokine production signature, a bulk cytokine or chemokine production profile, a bulk cytokine or chemokine production signal, a bulk cytokine or chemokine production score, a single cell cytokine or chemokine production profile, a single cell cytokine or chemokine production signature, a single cell cytokine or chemokine production profile, a single cell cytokine or chemokine production signal, a single cell cytokine or chemokine production score, a transcriptomic profile, a transcriptomic signature, a transcriptomic signature, a transcriptomic score, a pathway profile, a pathway signature, a pathway signal, a pathway score, a proteomic profile, a proteomic signature, a proteomic signal, a proteomic score, a genomic profile, a genomic signature, a genomic signal, a genomic score, a genomic stability profile, a genomic stability signature, a genomic stability signal, a genomic stability score, an epigenetic profile, an epigenetic signature, an epigenetic signal, an epigenetic score, a cell developmental potential assessment, a cell developmental potential profile, a cell developmental potential signature, a cell developmental potential score, a cell subtyping profile, a cell subtyping signature, a cell subtyping score, a cell receptor profile, a cell receptor signature, a cell receptor signal, a cell receptor score, immune cell identity profile, immune cell subtyping profile, cell subtype ratio profile, cell proliferation profile, cell proliferation score, HLA typing profile, and transcriptomic profile, or a combination thereof.

In some embodiments, the input data and/or the reference data comprises data corresponding to (i) one or more assay readouts, optionally cell activation, cell polyfunctionality, cell multifunctionality, cell cytotoxicity, cell growth rate, one or more characteristics associated with particular cell type, cellular activity associated with the cell or the population of cells, cell cytokine production, immune cell identity, immune cell subtyping, cell subtype ratio, cell proliferation, HLA typing, a transcriptome, or a combination thereof, and/or (ii) data relating to function (e.g., predicted function), optionally durability of cell growth, durability of cell response, cellular ability to elicit adaptive and innate immune responses, or a combination thereof; and optionally cell safety attributes; and/or cell impurity level(s).

In some embodiments, the input data and/or the reference data comprises one or more assay readouts and/or data relating to function (e.g., predicted function) from one or more assessments of the cells or the populations of cells or the reference cells or the reference populations of cells, optionally at one or more time points.

In some embodiments, the one or more qualitative assessments of one or more quantitative assay readouts are qualitative categorizations (e.g., poor, good, usable, or exceptional) (e.g., determined using a (e.g., computer-implemented) method disclosed herein).

In some embodiments, the reference data comprises persistence data, engraftment data, durability (e.g., of cell response) data, potency data, hypoimmunogenicity data, or a combination thereof (e.g., determined using a (e.g., computer-implemented) method disclosed herein).

In some embodiments, the reference cells and/or the populations of reference cells comprise suitable donor cells and/or unsuitable donor cells (e.g., determined using a (e.g., computer-implemented) method disclosed herein).

In some embodiments, the cell or the population of cells comprises a cell as disclosed herein.

In some embodiments, the reference cells and/or populations of reference cells do not have any modification.

In some embodiments, the reference cells and/or populations of reference cells have been modified (e.g., by a method disclosed herein).

In some embodiments, (i) the input data comprise data corresponding to at least one cell parameter and/or at least one function of the cell or the population of cells, (ii) the reference data comprise data corresponding to at least one cell parameter and/or at least one function of the reference cells and/or the populations of reference cells, or (iii) both (i) and (ii).

In some embodiments, (i) the input data comprises one or more quantitative assay readouts from the cell or the population of cells and the one or more quantitative assay readouts for the cell or the population of cells comprises one or more single values (e.g., one or more numerical values), one or more complex values (e.g., comprising a plurality of values) (e.g., comprising a range of values) [e.g., comprising multiple separate numerical values (e.g., pixel data from one or more images and/or flow plot data (e.g., from a gel))], one or more qualitative or semi-quantitative values [e.g., comprising one or more non-numerical values (e.g., a qualitative scale)], or a combination thereof (e.g., determined using a (e.g., computer-implemented) method disclosed herein), (ii) the reference data comprises one or more quantitative assay readouts from the reference cells and/or the populations of reference cells and the one or more quantitative assay readouts for the reference cells and/or the populations of reference cells comprises one or more single values (e.g., one or more numerical values), one or more complex values (e.g., comprising a plurality of values) (e.g., comprising a range of values) [e.g., comprising multiple separate numerical values (e.g., pixel data from one or more images and/or flow plot data (e.g., from a gel))], one or more qualitative or semi-quantitative values [e.g., comprising one or more non-numerical values (e.g., a qualitative scale)], or a combination thereof (e.g., determined using a (e.g., computer-implemented) method disclosed herein), or (iii) both (i) and (ii).

In some embodiments, the one or more quantitative assay readouts comprises the complex values and/or the qualitative or semi-quantitative values and the complex values and/or the qualitative or semi-quantitative values comprises pixel information from one or more images (e.g., wherein the one or more quantitative assay readouts comprises one or more images from one or more assays), flow plot data (e.g., wherein the one or more quantitative assay readouts comprises one or more images from one or more assays), or a combination thereof (e.g., determined using a (e.g., computer-implemented) method disclosed herein).

In some embodiments, the input data and/or the reference data comprises one or more single values that have been converted from one or more complex values and/or qualitative or semi-quantitative values of one or more quantitative assay readouts (e.g., from the cell or the population of cells and/or from the reference cells and/or the populations of reference cells) (e.g., prior to determining the predicted function) [e.g., using a predetermined conversion scheme (e.g., scale) (e.g., by combining (e.g., concatenating) and/or statistically processing (e.g., averaging), by the one or more processors, the values)].

In some embodiments, the one or more quantitative assay readouts (e.g., from the cell or the population of cells and/or from the reference cells and/or the populations of reference cells) comprise the complex values and/or the qualitative or semi-quantitative values and the method comprises converting, by the one or more processors, the complex values and/or the qualitative or semi-quantitative values to a single value for each of the one or more quantitative assay readouts (e.g., prior to determining the predicted function) [e.g., using a predetermined conversion scheme (e.g., scale)(e.g., by combining (e.g., concatenating) and/or statistically processing (e.g., averaging), by the one or more processors, the values)].

In some embodiments, determining the predicted function comprises comparing, by the one or more processors, one or more single values for one or more quantitative assay readouts for the cell or the population of cells and one or more single values for one or more quantitative assay readouts for the reference cells and/or the populations of reference cells (e.g., after converting, by the one or more processors, to the single value(s) from one or more complex values and/or one or more qualitative and/or semi-quantitative values).

In some embodiments, the method comprises converting, by the one or more processors, one or more quantitative assay readouts into a suitable format for performing the determining (e.g., for input into a machine learning module) [e.g., suitable data format and/or size (e.g., string and/or number format and/or size)].

In some embodiments, determining the predicted function comprises using one or more weights to weight the input data and/or the reference data (e.g., relative to each other and/or to differently weight different portions of the input data and/or to differently weight different portions of the reference data) (e.g., used in a loss function used in a machine learning module) [e.g., that have been determined by a trained algorithm (e.g., in a machine learning module)].

In some embodiments, the method comprises determining, by the one or more processors, the one or more weights.

In some embodiments, the one or more weights are for weighting one or more quantitative assay readouts comprised in the input data and/or the reference data.

In some embodiments, determining the one or more weights comprises receiving, by the one or more processors, user input corresponding to the one or more weights (e.g., via one or more graphical user interfaces (GUIs)).

In some embodiments, the user input comprises one or more of the one or more weights and/or one or more cell characteristics (e.g., function(s) and/or parameter(s)) to weight.

In some embodiments, determining the one or more weights comprises determining the one or more weights using a trained algorithm or training an algorithm (e.g., in a machine learning module).

In some embodiments, the one or more weights correspond to (e.g., wherein the user input represents): (i) one or more cell characteristics to consider or not consider (e.g., one or more quantitative assay readouts to consider or not consider), (ii) a preference of one or more cell characteristics over one or more other cell characteristics [e.g., a ranked list of two or more cell characteristics (e.g., a ranked list of some but not all or all available cell characteristics to choose from)] (e.g., a preference of one or more quantitative assay readouts over one or more other assay readouts), (iii) a selection of one or more subsets of the input data and/or the reference data to weight more heavily or less heavily (e.g., comprising one or more desired weights [e.g., that is/are then fixed or that serves as an initial value that is/are then modified (e.g., through iteration in a machine learning module)], or (iv) a combination thereof.

In some embodiments, the method comprises comparing, by the one or more processors, the input data and (e.g., to) the reference data (e.g., using a machine learning module).

In some embodiments, comparing the input data and the reference data comprises performing, by the one or more processors, a multiparametric comparison between the input data and the reference data.

In some embodiments, the method comprises comparing (e.g., optimizing), by the one or more processors, two or more, three or more, four or more, five or more, six or more, seven or more, eight or more, nine or more, or ten or more parameters.

In some embodiments, the multiparametric comparison is a biomarker.

In some embodiments, the predicted function comprises one or more of functions selected from the group consisting of durability of cell growth, durability of cell response, and cellular ability to elicit adaptive and innate immune responses.

In some embodiments, the method comprises determining (e.g., classifying and/or inferring), by the one or more processors, (e.g., wherein a machine learning module that determines the predicted function outputs) a categorization of the cell or the population of cells that is indicative of the predicted function of the cell or the population of cells.

In some embodiments, the predicted function comprises a categorization of the cell or the population of cells.

In some embodiments, the categorization is a qualitative categorization (e.g., that uses a qualitative scale comprising one or more of: ‘poor,’ ‘usable,’ ‘good,’ and ‘exceptional’).

In some embodiments, the categorization uses a quantitative scale (e.g., provides a numerical value on a predetermined scale).

In some embodiments, the categorization is indicative of a suitability of the cell or the population of cells for use as donor cell(s) in a cell therapy.

In some embodiments, the method comprises determining (e.g., classifying and/or inferring), by the one or more processors, suitability of the cell or the population of cells for use as donor cell(s) in a cell therapy (e.g., wherein the predicted function is indicative of the suitability) (e.g., wherein the suitability is determined using the predicted function) (e.g., using the input data and the reference data in a (e.g., computer-implemented) method to determine suitability as disclosed herein).

In some embodiments, determining the suitability comprises determining (e.g., classifying and/or inferring), by the one or more processors, that the cell or the population of cells is/are suitable for use as donor cell(s) (e.g., by a (e.g., computer-implemented) method disclosed herein).

In some embodiments, the input data comprises data corresponding to one or more cell characteristics (e.g., function(s) and/or parameter(s)) and the method comprises determining (e.g., classifying and/or inferring), by the one or more processors that the cell or the population of cells is/are suitable for use as donor cell(s) due, in part, to presence and/or absence of one or more traits from the one or more cell characteristics (e.g., by a (e.g., computer-implemented) method disclosed herein).

In some embodiments, wherein the one or more cell characteristics correspond to age of subject from which the cell or the population of cells is derived, health history [e.g., health status (e.g., current health status)] of the subject, sex of the subject, or a combination thereof [e.g., and the one or more traits correspond to an acceptable and/or desirable (e.g., preferred) age or age range, acceptable and/or desirable (e.g., preferred) sex, presence of one or more acceptable and/or desirable (e.g., preferred) health traits, absence of one or more unacceptable and/or undesirable (e.g., non-preferred) health traits, or a combination thereof].

In some embodiments, the method comprises determining (e.g., classifying and/or inferring), by the one or more processors, that the cell or the population of cells is suitable due to the presence and/or absence of the one or more traits when the cell or the population of cells would otherwise be less suitable or unsuitable without and/or with having the one or more traits, respectively (e.g., by a (e.g., computer-implemented) method disclosed herein).

In some embodiments, wherein determining the suitability comprises using one or more weights to weight the input data and/or the reference data (e.g., relative to each other and/or to differently weight different portions of the input data and/or to differently weight different portions of the reference data) (e.g., used in a loss function used in a machine learning module).

In some embodiments, the method comprises determining, by the one or more processors, one or more weights for use in determining the suitability (e.g., used in a loss function used in a machine learning module) [e.g., wherein determining the one or more weights comprises (i) determining the one or more weights using a trained algorithm or (ii) training an algorithm (e.g., in a machine learning module)].

In some embodiments, the one or more weights correspond to one or more desirable cell characteristics and/or the one or more weights have been selected (e.g., by user input) to more heavily weight one or more desirable cell characteristics than one or more other cell characteristics (e.g., wherein the one or more desirable cell characteristics are or have been determined, by the one or more processors, using reference data that comprises cell performance data).

In some embodiments, the one or more weights are for weighting one or more quantitative assay readouts comprised in the input data and/or the reference data.

In some embodiments, determining the one or more weights comprises receiving, by the one or more processors, user input corresponding to the one or more weights (e.g., via one or more graphical user interfaces (GUIs)).

In some embodiments, the user input comprises one or more of the one or more weights and/or one or more cell characteristics to weight.

In some embodiments, the one or more weights correspond to (e.g., wherein the user input represents): (i) one or more cell characteristics to consider or not consider (e.g., one or more quantitative assay readouts to consider or not consider), (ii) a preference of one or more cell characteristics over one or more other cell characteristics [e.g., a ranked list of two or more cell characteristics (e.g., a ranked list of some but not all or all available cell characteristics to choose from)] (e.g., a preference of one or more quantitative assay readouts over one or more other assay readouts), (iii) a selection of one or more subsets of the input data and/or the reference data to weight more heavily or less heavily (e.g., comprising one or more desired weights [e.g., that is/are then fixed or that serves as an initial value that is/are then modified (e.g., through iteration in a machine learning module)], or (iv) a combination thereof.

In some embodiments, the system comprises a machine learning module (e.g., stored in the memory), wherein the method is performed using (e.g., wherein the predicted function of the cell or the population of cells is determined using) the machine learning module that (a) has been trained using the reference data and (b) receives the input data as an input [e.g., in addition to one or more other inputs (e.g., user-selected weight(s) to be applied (e.g., in a loss function))].

In some embodiments, the machine learning module employs a regression-based model (e.g., a logistic regression model), a regularization-based model (e.g., an elastic net model or a ridge regression model), an instance-based model (e.g., a support vector machine or a k-nearest neighbor model), a Bayesian-based model (e.g., a naïve-based model or a Gaussian naive-based model), a clustering-based model (e.g., an expectation maximization model), an ensemble-based model (e.g., an adaptive boosting model, a random forest model, a bootstrap-aggregation model, or a gradient boosting machine model), or a neural-network-based model (e.g., a convolutional neural network, a recurrent neural network, autoencoder, a back propagation network, or a stochastic gradient descent network).

In one aspect, the present disclosure provides a non-transitory computer-readable storage medium storing one or more programs, the one or more programs comprising instructions, which when executed by one or more processors of a computing device, cause the device to implement a method comprising: receiving, by one or more processors, input data corresponding to the cell or the population of cells; and determining (e.g., inferring), by the one or more processors, a predicted function of the cell or the population of cells using the input data and reference data corresponding to reference cells and/or populations of reference cells (e.g., by comparing the input data and the reference data).

In one aspect, the present disclosure provides a non-transitory computer-readable storage medium storing one or more programs, the one or more programs comprising instructions, which when executed by one or more processors of a computing device, cause the device to implement a method comprising: receiving, by one or more processors, input data corresponding to the cell or the population of cells; and determining (e.g., inferring), by the one or more processors, a predicted in vivo function of the cell or the population of cells using the input data and reference data corresponding to reference cells and/or populations of reference cells [e.g., by comparing the input data and the reference data].

In one aspect, the present disclosure provides a non-transitory computer-readable storage medium storing one or more programs, the one or more programs comprising instructions, which when executed by one or more processors of a computing device, cause the device to implement a method comprising training, by one or more processors, an algorithm (e.g., in a machine learning module) using reference data corresponding to reference cells and/or populations of reference cells (e.g., obtained using a method disclosed herein).

In one aspect, the present disclosure provides a non-transitory computer-readable storage medium storing one or more programs, the one or more programs comprising instructions, which when executed by one or more processors of a computing device, cause the device to implement a method comprising training, by one or more processors, an algorithm (e.g., in a machine learning module) using reference data corresponding to reference cells and/or populations of reference cells.

In one aspect, the present disclosure provides a non-transitory computer-readable storage medium storing one or more programs, the one or more programs comprising instructions, which when executed by one or more processors of a computing device, cause the device to implement a method comprising comparing, by one or more processors, reference data generated from reference cells and/or populations of reference cells [e.g., using an algorithm (e.g., in a machine learning module)].

In some embodiments, (i) the reference data comprises one or more quantitative assay readouts from reference cells and/or the populations of reference cells (e.g., determined using a (e.g., computer-implemented) method disclosed herein), one or more qualitative assessments of one or more quantitative assay readouts from reference cells and/or the populations of reference cells (e.g., determined using a (e.g., computer-implemented) method in accordance with any preceding embodiment), or a combination thereof, (ii) the input data comprises one or more quantitative assay readouts from the cell or the population of cells (e.g., determined using a (e.g., computer-implemented) method disclosed herein), one or more qualitative assessments of one or more quantitative assay readouts from the cell or the population of cells (e.g., determined using a (e.g., computer-implemented) method disclosed herein), or a combination thereof, or (iii) both (i) and (ii).

In some embodiments, the input data and/or the reference data comprises data corresponding to age of subject(s)(e.g., from which the cell or the population of cells is derived and/or from which the reference cells and/or the populations of reference cells are derived, respectively), health history [e.g., health status (e.g., current health status)] of the subject(s), sex of the subject(s), or a combination thereof.

In some embodiments, the reference data comprises (e.g., further comprises) clinical output data, in vitro experimental data, in vivo experimental data, or a combination thereof (e.g., determined using a (e.g., computer-implemented) method disclosed herein).

In some embodiments, the input data and/or the reference data comprises one or more quantitative assay readouts from one or more assays and the one or more assays comprise an in vitro assay, an in vivo assay, an immune assay, a cell activity assay, a cell avidity assay, a cell proliferation assay, a cell cytotoxicity assay, a cellular stress assay, a tumor challenge assay, an expression assay, a cytokine production assay, transcriptomic profiling assay, a proteomic profiling assay, a genomic profiling assay, a genomic stability assay, an epigenetic profiling assay, a cell developmental potential profiling assay, a cell subtyping assay, a cell receptor profiling assay or a combination thereof.

In some embodiments, the one or more quantitative assay readouts comprises a cell functionality score, a cell polyfunctionality index, a cell multifunctionality index, an in vivo efficacy score, an in vivo activity score, an in vivo response score, an in vitro efficacy score, an in vitro activity score, an in vitro response score, an immune efficacy score, an immune activity score, an immune response score, a cell activity score, a cell activity response score, a cell specificity score, a cell sensitivity score, a cell avidity score, a cell proliferation score, a cell proliferative index, a cell cytotoxicity score, a cell cytotoxicity response score, a cell stress score, a cell stress response score, a tumor challenge efficacy score, a tumor challenge activity score, a tumor challenge response score, a tumor challenge specificity score, a tumor challenge sensitivity score, an expression profile, an expression signature, an expression signal, an expression score, a bulk cytokine or chemokine production profile, a bulk cytokine or chemokine production signature, a bulk cytokine or chemokine production profile, a bulk cytokine or chemokine production signal, a bulk cytokine or chemokine production score, a single cell cytokine or chemokine production profile, a single cell cytokine or chemokine production signature, a single cell cytokine or chemokine production profile, a single cell cytokine or chemokine production signal, a single cell cytokine or chemokine production score, a transcriptomic profile, a transcriptomic signature, a transcriptomic signature, a transcriptomic score, a pathway profile, a pathway signature, a pathway signal, a pathway score, a proteomic profile, a proteomic signature, a proteomic signal, a proteomic score, a genomic profile, a genomic signature, a genomic signal, a genomic score, a genomic stability profile, a genomic stability signature, a genomic stability signal, a genomic stability score, an epigenetic profile, an epigenetic signature, an epigenetic signal, an epigenetic score, a cell developmental potential assessment, a cell developmental potential profile, a cell developmental potential signature, a cell developmental potential score, a cell subtyping profile, a cell subtyping signature, a cell subtyping score, a cell receptor profile, a cell receptor signature, a cell receptor signal, a cell receptor score, immune cell identity profile, immune cell subtyping profile, cell subtype ratio profile, cell proliferation profile, cell proliferation score, HLA typing profile, and transcriptomic profile, or a combination thereof.

In some embodiments, the input data and/or the reference data comprises data corresponding to (i) one or more assay readouts, optionally cell activation, cell polyfunctionality, cell multifunctionality, cell cytotoxicity, cell growth rate, one or more characteristics associated with particular cell type, cellular activity associated with the cell or the population of cells, cell cytokine production, immune cell identity, immune cell subtyping, cell subtype ratio, cell proliferation, HLA typing, a transcriptome, or a combination thereof, and/or (ii) data relating to function (e.g., predicted function), optionally durability of cell growth, durability of cell response, cellular ability to elicit adaptive and innate immune responses, or a combination thereof; and optionally cell safety attributes; and/or cell impurity level(s).

In some embodiments, the input data and/or the reference data comprises one or more assay readouts and/or data relating to function (e.g., predicted function) from one or more assessments of the cells or the populations of cells or the reference cells or the reference populations of cells, optionally at one or more time points.

In some embodiments, the one or more qualitative assessments of one or more quantitative assay readouts are qualitative categorizations (e.g., poor, good, usable, or exceptional) (e.g., determined using a (e.g., computer-implemented) method disclosed herein).

In some embodiments, the reference data comprises persistence data, engraftment data, durability (e.g., of cell response) data, potency data, hypoimmunogenicity data, or a combination thereof (e.g., determined using a (e.g., computer-implemented) method disclosed herein).

In some embodiments, the reference cells and/or the populations of reference cells comprise suitable donor cells and/or unsuitable donor cells (e.g., determined using a (e.g., computer-implemented) method disclosed herein).

In some embodiments, the cell or the population of cells comprises a cell as disclosed herein.

In some embodiments, the reference cells and/or populations of reference cells do not have any modification.

In some embodiments, the reference cells and/or populations of reference cells have been modified (e.g., by a method as disclosed herein).

In some embodiments, (i) the input data comprise data corresponding to at least one cell parameter and/or at least one function of the cell or the population of cells, (ii) the reference data comprise data corresponding to at least one cell parameter and/or at least one function of the reference cells and/or the populations of reference cells, or (iii) both (i) and (ii).

In some embodiments, (i) the input data comprises one or more quantitative assay readouts from the cell or the population of cells and the one or more quantitative assay readouts for the cell or the population of cells comprises one or more single values (e.g., one or more numerical values), one or more complex values (e.g., comprising a plurality of values) (e.g., comprising a range of values) [e.g., comprising multiple separate numerical values (e.g., pixel data from one or more images and/or flow plot data (e.g., from a gel))], one or more qualitative or semi-quantitative values [e.g., comprising one or more non-numerical values (e.g., a qualitative scale)], or a combination thereof (e.g., determined using a (e.g., computer-implemented) method disclosed herein), (ii) the reference data comprises one or more quantitative assay readouts from the reference cells and/or the populations of reference cells and the one or more quantitative assay readouts for the reference cells and/or the populations of reference cells comprises one or more single values (e.g., one or more numerical values), one or more complex values (e.g., comprising a plurality of values) (e.g., comprising a range of values) [e.g., comprising multiple separate numerical values (e.g., pixel data from one or more images and/or flow plot data (e.g., from a gel))], one or more qualitative or semi-quantitative values [e.g., comprising one or more non-numerical values (e.g., a qualitative scale)], or a combination thereof (e.g., determined using a (e.g., computer-implemented) method disclosed herein), or (iii) both (i) and (ii).

In some embodiments, the one or more quantitative assay readouts comprises the complex values and/or the qualitative or semi-quantitative values and the complex values and/or the qualitative or semi-quantitative values comprises pixel information from one or more images (e.g., wherein the one or more quantitative assay readouts comprises one or more images from one or more assays), flow plot data (e.g., wherein the one or more quantitative assay readouts comprises one or more images from one or more assays), or a combination thereof (e.g., determined using a (e.g., computer-implemented) method disclosed herein).

In some embodiments, the input data and/or the reference data comprises one or more single values that have been converted from one or more complex values and/or qualitative or semi-quantitative values of one or more quantitative assay readouts (e.g., from the cell or the population of cells and/or from the reference cells and/or the populations of reference cells) (e.g., prior to determining the predicted function) [e.g., using a predetermined conversion scheme (e.g., scale) (e.g., by combining (e.g., concatenating) and/or statistically processing (e.g., averaging), by the one or more processors, the values)].

In some embodiments, the one or more quantitative assay readouts (e.g., from the cell or the population of cells and/or from the reference cells and/or the populations of reference cells) comprise the complex values and/or the qualitative or semi-quantitative values and the method comprises converting, by the one or more processors, the complex values and/or the qualitative or semi-quantitative values to a single value for each of the one or more quantitative assay readouts (e.g., prior to determining the predicted function) [e.g., using a predetermined conversion scheme (e.g., scale)(e.g., by combining (e.g., concatenating) and/or statistically processing (e.g., averaging), by the one or more processors, the values)].

In some embodiments, determining the predicted function comprises comparing, by the one or more processors, one or more single values for one or more quantitative assay readouts for the cell or the population of cells and one or more single values for one or more quantitative assay readouts for the reference cells and/or the populations of reference cells (e.g., after converting, by the one or more processors, to the single value(s) from one or more complex values and/or one or more qualitative and/or semi-quantitative values).

In some embodiments, the method comprises converting, by the one or more processors, one or more quantitative assay readouts into a suitable format for performing the determining (e.g., for input into a machine learning module) [e.g., suitable data format and/or size (e.g., string and/or number format and/or size)].

In some embodiments, determining the predicted function comprises using one or more weights to weight the input data and/or the reference data (e.g., relative to each other and/or to differently weight different portions of the input data and/or to differently weight different portions of the reference data) (e.g., used in a loss function used in a machine learning module) [e.g., that have been determined by a trained algorithm (e.g., in a machine learning module)].

In some embodiments, the method comprises determining, by the one or more processors, the one or more weights.

In some embodiments, the one or more weights are for weighting one or more quantitative assay readouts comprised in the input data and/or the reference data.

In some embodiments, determining the one or more weights comprises receiving, by the one or more processors, user input corresponding to the one or more weights (e.g., via one or more graphical user interfaces (GUIs)).

In some embodiments, the user input comprises one or more of the one or more weights and/or one or more cell characteristics (e.g., function(s) and/or parameter(s)) to weight.

In some embodiments, determining the one or more weights comprises determining the one or more weights using a trained algorithm or training an algorithm (e.g., in a machine learning module).

In some embodiments, the one or more weights correspond to (e.g., wherein the user input represents): (i) one or more cell characteristics to consider or not consider (e.g., one or more quantitative assay readouts to consider or not consider), (ii) a preference of one or more cell characteristics over one or more other cell characteristics [e.g., a ranked list of two or more cell characteristics (e.g., a ranked list of some but not all or all available cell characteristics to choose from)] (e.g., a preference of one or more quantitative assay readouts over one or more other assay readouts), (iii) a selection of one or more subsets of the input data and/or the reference data to weight more heavily or less heavily (e.g., comprising one or more desired weights [e.g., that is/are then fixed or that serves as an initial value that is/are then modified (e.g., through iteration in a machine learning module)], or (iv) a combination thereof.

In some embodiments, the method comprises comparing, by the one or more processors, the input data and (e.g., to) the reference data (e.g., using a machine learning module).

In some embodiments, comparing the input data and the reference data comprises performing, by the one or more processors, a multiparametric comparison between the input data and the reference data.

In some embodiments, the method comprises comparing (e.g., optimizing), by the one or more processors, two or more, three or more, four or more, five or more, six or more, seven or more, eight or more, nine or more, or ten or more parameters.

In some embodiments, the multiparametric comparison is a biomarker.

In some embodiments, the predicted function comprises one or more of functions selected from the group consisting of durability of cell growth, durability of cell response, and cellular ability to elicit adaptive and innate immune responses.

In some embodiments, the method comprises determining (e.g., classifying and/or inferring), by the one or more processors, (e.g., wherein a machine learning module that determines the predicted function outputs) a categorization of the cell or the population of cells that is indicative of the predicted function of the cell or the population of cells.

In some embodiments, the predicted function comprises a categorization of the cell or the population of cells.

In some embodiments, the categorization is a qualitative categorization (e.g., that uses a qualitative scale comprising one or more of: ‘poor,’ ‘usable,’ ‘good,’ and ‘exceptional’).

In some embodiments, the categorization uses a quantitative scale (e.g., provides a numerical value on a predetermined scale).

In some embodiments, the categorization is indicative of a suitability of the cell or the population of cells for use as donor cell(s) in a cell therapy.

In some embodiments, determining (e.g., classifying and/or inferring), by the one or more processors, suitability of the cell or the population of cells for use as donor cell(s) in a cell therapy (e.g., wherein the predicted function is indicative of the suitability) (e.g., wherein the suitability is determined using the predicted function) (e.g., using the input data and the reference data in a (e.g., computer-implemented) method to determine suitability as disclosed herein).

In some embodiments, determining the suitability comprises determining (e.g., classifying and/or inferring), by the one or more processors, that the cell or the population of cells is/are suitable for use as donor cell(s) (e.g., by a (e.g., computer-implemented) method disclosed herein).

In some embodiments, the input data comprises data corresponding to one or more cell characteristics (e.g., function(s) and/or parameter(s)) and the method comprises determining (e.g., classifying and/or inferring), by the one or more processors that the cell or the population of cells is/are suitable for use as donor cell(s) due, in part, to presence and/or absence of one or more traits from the one or more cell characteristics (e.g., by a (e.g., computer-implemented) method disclosed herein).

In some embodiments, wherein the one or more cell characteristics correspond to age of subject from which the cell or the population of cells is derived, health history [e.g., health status (e.g., current health status)] of the subject, sex of the subject, or a combination thereof [e.g., and the one or more traits correspond to an acceptable and/or desirable (e.g., preferred) age or age range, acceptable and/or desirable (e.g., preferred) sex, presence of one or more acceptable and/or desirable (e.g., preferred) health traits, absence of one or more unacceptable and/or undesirable (e.g., non-preferred) health traits, or a combination thereof].

In some embodiments, wherein the method comprises determining (e.g., classifying and/or inferring), by the one or more processors, that the cell or the population of cells is suitable due to the presence and/or absence of the one or more traits when the cell or the population of cells would otherwise be less suitable or unsuitable without and/or with having the one or more traits, respectively (e.g., by a (e.g., computer-implemented) method disclosed herein).

In some embodiments, determining the suitability comprises using one or more weights to weight the input data and/or the reference data (e.g., relative to each other and/or to differently weight different portions of the input data and/or to differently weight different portions of the reference data)(e.g., used in a loss function used in a machine learning module).

In some embodiments, the method comprises determining, by the one or more processors, one or more weights for use in determining the suitability (e.g., used in a loss function used in a machine learning module).

In some embodiments, the one or more weights correspond to one or more desirable cell characteristics and/or the one or more weights have been selected (e.g., by user input) to more heavily weight one or more desirable cell characteristics than one or more other cell characteristics (e.g., wherein the one or more desirable cell characteristics are or have been determined, by the one or more processors, using reference data that comprises cell performance data).

In some embodiments, the one or more weights are for weighting one or more quantitative assay readouts comprised in the input data and/or the reference data.

In some embodiments, determining the one or more weights comprises receiving, by the one or more processors, user input corresponding to the one or more weights (e.g., via one or more graphical user interfaces (GUIs)).

In some embodiments, the user input comprises one or more of the one or more weights and/or one or more cell characteristics to weight.

In some embodiments, the one or more weights correspond to (e.g., wherein the user input represents): (i) one or more cell characteristics to consider or not consider (e.g., one or more quantitative assay readouts to consider or not consider). (ii) a preference of one or more cell characteristics over one or more other cell characteristics [e.g., a ranked list of two or more cell characteristics (e.g., a ranked list of some but not all or all available cell characteristics to choose from)] (e.g., a preference of one or more quantitative assay readouts over one or more other assay readouts), (iii) a selection of one or more subsets of the input data and/or the reference data to weight more heavily or less heavily (e.g., comprising one or more desired weights [e.g., that is/are then fixed or that serves as an initial value that is/are then modified (e.g., through iteration in a machine learning module)], or (iv) a combination thereof.

In some embodiments, method is performed using (e.g., wherein the predicted function of the cell or the population of cells is determined using) a machine learning module (e.g., stored in the medium) that (a) has been trained using the reference data and (b) receives the input data as an input [e.g., in addition to one or more other inputs (e.g., user-selected weight(s) to be applied (e.g., in a loss function))].

In some embodiments, the machine learning module employs a regression-based model (e.g., a logistic regression model), a regularization-based model (e.g., an elastic net model or a ridge regression model), an instance-based model (e.g., a support vector machine or a k-nearest neighbor model), a Bayesian-based model (e.g., a naive-based model or a Gaussian naive-based model), a clustering-based model (e.g., an expectation maximization model), an ensemble-based model (e.g., an adaptive boosting model, a random forest model, a bootstrap-aggregation model, or a gradient boosting machine model), or a neural-network-based model (e.g., a convolutional neural network, a recurrent neural network, autoencoder, a back propagation network, or a stochastic gradient descent network).

In one aspect, the present disclosure provides a cell therapy product comprising a population of cells, wherein
i. the population includes cells with hypoimmune gene modifications (HIP cells) that exhibit reduced expression of one or more molecules of the MHC class I and/or MHC class II molecules and optionally also exhibit increased expression of at least one tolerogenic factor; and
ii. at least 30%, at least 40%, at least 50%, at least 60%, at least 70%, at least 80%, or at least 90% of cells in the population exhibit reduced expression of one or more molecules of the MHC class I and/or MHC class II molecules and optionally also exhibit increased expression of at least one tolerogenic factor.

In one aspect, the present disclosure provides a pharmaceutical composition comprising a population of cells, wherein:
i. the population includes cells with hypoimmune gene modifications (HIP cells) that exhibit reduced expression of one or more molecules of the MHC class I and/or MHC class II molecules and optionally also exhibit increased expression of at least one tolerogenic factor; and
ii. at least 30%, at least 40%, at least 50%, at least 60%, at least 70%, at least 80%, or at least 90% of cells in the population exhibit reduced expression of one or more molecules of the MHC class I and/or MHC class II molecules and optionally also exhibit increased expression of at least one tolerogenic factor.

In some embodiments of these aspects, at least 5%, at least 10%, at least 20%, at least 30%, at least 40%, at least 50%, at least 60%, at least 70% of cells in the population of cells do not exhibit reduced expression of one or more molecules of the MHC class I and/or MHC class II molecules and optionally also do not exhibit increased expression of at least one tolerogenic factor. In some embodiments, 30-90%, 30-80%, 30-70%, 30-60%, 30-50% or 40-50% of cells in the population of cells are HIP cells that exhibit reduced expression of one or more molecules of the MHC class I and/or MHC class II molecules and optionally increased expression of at least one tolerogenic factor.

In some embodiments of these aspects, the HIP cells are as described herein. In some embodiments, the HIP cells further comprise a suicide gene or suicide switch as described herein.

In some embodiments of these aspects, the cells are neural cells as described herein.

In some embodiments of these aspects, the cells are beta cells as described herein.

In some embodiments of these aspects, the cells are hepatocytes as described herein.

In some embodiments of these aspects, the cells are endothelial cells as described herein.

In some embodiments of these aspects, the cells are epithelial cells as described herein.

In some embodiments of these aspects, the cells are cardiac cells (e.g., cardiomyocytes) as described herein.

In some embodiments of these aspects, 70-100%, 80-100%, or 90-100% of cells in the population of cells express a CAR. In some embodiments, the cells are T cells as described herein. In some embodiments, the cells are NK cells as described herein. In some embodiments, the cells are macrophages as described herein. In some embodiments, the cells are B cells as described herein.

In one aspect, the present disclosure provides a cell therapy product as described herein, or a pharmaceutical composition as described herein, for use in therapy. In one aspect, the present disclosure provides a cell therapy product as described herein, or a pharmaceutical composition as described herein, for use in a method of treating a disease or condition. In one aspect, the present disclosure provides a method of treating a disease or condition in a subject comprising administering to the subject a cell therapy product as described herein, or a pharmaceutical composition as described anywhere herein. In some embodiments of these aspects, the subject to be treated is not immunosuppressed i.e., has not been administered one or more immunosuppressive agents. In some embodiments of these aspects, the cell and the disease or condition are as described anywhere herein.

In one aspect, the present disclosure provides a cell therapy product as described herein below:

In this aspect, the disclosure provides a method wherein said method comprises:
a. isolating cells from a donor or population of donors; and
b. manipulating the isolated cells to produce a cell therapy product.

In some embodiments, the method further comprises cryopreserving the cell therapy product, and cell viability of the cell therapy product after cryopreservation is at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.

In some embodiments, the cell viability of the cell therapy product after cryopreservation is at least about 80%.

In some embodiments, the cell viability of the cell therapy product after cryopreservation is at least about 90%.

In some embodiments, the cell viability of the cell therapy product after cryopreservation is at least about 95%.

In some embodiments, the manipulation comprises transducing the isolated cells with a viral vector, and transduction efficiency of the isolated cells is at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.

In some embodiments, transduction efficiency of the isolated cells is at least about 40%.

In some embodiments, transduction efficiency of the isolated cells is at least about 45%.

In some embodiments, transduction efficiency of the isolated cells is at least about 50%.

In some embodiments, transduction efficiency of the isolated cells is at least about 55%.

In some embodiments, transduction efficiency of the isolated cells is at least about 60%.

In some embodiments, transduction efficiency of the isolated cells is at least about 65%.

In some embodiments, the manipulation comprises transducing the isolated cells with a viral vector, and viral copy number (VCN) in the transduced cells is no more than about 5.0, no more than about 4.7, no more than about 4.5, no more than about 4.2, no more than about 4.0, no more than about 3.7, no more than about 3.5, no more than about 3.2, no more than about 3.0, no more than about 2.7, no more than about 2.5, no more than about 2.2, no more than about 2, no more than about 1.7, no more than about 1.5, no more than about 1.2, no more than about 1.0, no more than about 0.7, no more than about 0.5, no more than about 0.2, or no more than about 0.1.

In some embodiments, the VCN in the transduced cells is no more than about 3.0.

In some embodiments, the VCN in the transduced cells is no more than about 2.7.

In some embodiments, the VCN in the transduced cells is no more than about 2.5.

In some embodiments, the VCN in the transduced cells is no more than about 2.2.

In some embodiments, the VCN in the transduced cells is no more than about 2.0.

In some embodiments, the manipulation comprises modifying the genome of the isolated cells by inactivating or disrupting the TRAC gene locus and/or the TRBC gene locus and selecting the genome modified cells.

In some embodiments, the inactivation or disruption comprises inactivation or disruption of:
a. one TRAC allele or both TRAC alleles, and/or
b. one TRBC allele or both TRBC alleles.

In some embodiments, the percentage of residual TCRαβ+ cells in the selected cells is no more than about 1.0%, no more than about 0.9%, no more than about 0.8%, no more than about 0.7%, no more than about 0.6%, no more than about 0.5%, no more than about 0.4%, no more than about 0.3%, no more than about 0.2%, or no more than about 0.1%.

In some embodiments, the percentage of residual TCRαβ+ cells in the selected cells is no more than about 0.5%.

In some embodiments, the percentage of residual TCRαβ+ cells in the selected cells is no more than about 0.4%.

In some embodiments, the percentage of residual TCRαβ+ cells in the selected cells is no more than about 0.3%.

In some embodiments, the percentage of residual TCRαβ+ cells in the selected cells is no more than about 0.2%.

In some embodiments, the percentage of residual TCRαβ+ cells in the selected cells is no more than about 0.1%.

In some embodiments, the manipulation comprises preparing the cell therapy product with CD4+ cells and/or CD8+ cells.

In some embodiments, the final percentage of CD4+ cells in the cell therapy product is at least about 20%, at least about 25%, at least about 30%, at least about 35%, at least about 40%, at least about 45%, at least about 50%, at least about 55%, at least about 60%, at least about 65%, at least about 70%, or at least about 75%.

In some embodiments, the final percentage of CD8+ cells in the cell therapy product is at least about 20%, at least about 25%, at least about 30%, at least about 35%, at least about 40%, at least about 45%, at least about 50%, at least about 55%, at least about 60%, at least about 65%, at least about 70%, or at least about 75%.

In some embodiments, the final percentage of CD4+ cells in the cell therapy product is at least about 30%.

In some embodiments, the final percentage of CD4+ cells in the cell therapy product is at least about 35%.

In some embodiments, the final percentage of CD4+ cells in the cell therapy product is at least about 40%.

In some embodiments, the final percentage of CD4+ cells in the cell therapy product is at least about 45%.

In some embodiments, the final percentage of CD4+ cells in the cell therapy product is at least about 50%.

In some embodiments, the final percentage of CD4+ cells in the cell therapy product is at least about 55%.

In some embodiments, the final percentage of CD4+ cells in the cell therapy product is at least about 60%.

In some embodiments, the final percentage of CD4+ cells in the cell therapy product is at least about 65%.

In some embodiments, the final percentage of CD8+ cells in the cell therapy product is at least about 30%.

In some embodiments, the final percentage of CD8+ cells in the cell therapy product is at least about 35%.

In some embodiments, the final percentage of CD8+ cells in the cell therapy product is at least about 40%.

In some embodiments, the final percentage of CD8+ cells in the cell therapy product is at least about 45%.

In some embodiments, the final percentage of CD8+ cells in the cell therapy product is at least about 50%.

In some embodiments, the final percentage of CD8 cells in the cell therapy product is at least about 55%.

In some embodiments, the final percentage of CD8 cells in the cell therapy product is at least about 60%.

In some embodiments, the final percentage of CD8 cells in the cell therapy product is at least about 65%.

In some embodiments, the final percentage of CD8 cells in the cell therapy product is at least about 70%.

In some embodiments, the final ratio of CD4+ cells to CD8+ cells in the cell therapy product is about 0.4:1, about 0.45:1, about 0.5:1, about 0.55:1, about 0.6:1, about 0.65:1, about 0.7:1, about 0.75:1, about 0.8:1, about 0.85:1, about 0.9:1, about 0.95:1, about 1:1, about 1:0.95, about 1:0.9, about 1:0.85, about 1:0.8, about 1:0.75, about 1:0.7, about 1:0.65, about 1:0.6, about 1:0.55, about 1:0.5, about 1:0.45, or about 1:0.4.

In some embodiments, the final ratio of CD4+ cells to CD8+ cells in the cell therapy product is about 0.45:1.

In some embodiments, the final ratio of CD4+ cells to CD8+ cells in the cell therapy product is about 0.50:1.

In some embodiments, the final ratio of CD4+ cells to CD8+ cells in the cell therapy product is about 0.55:1.

In some embodiments, the final ratio of CD4+ cells to CD8+ cells in the cell therapy product is about 0.60:1.

In some embodiments, the final ratio of CD4+ cells to CD8+ cells in the cell therapy product is about 0.65:1.

In some embodiments, the final ratio of CD4+ cells to CD8+ cells in the cell therapy product is about 1:0.60.

In some embodiments, the final ratio of CD4+ cells to CD8+ cells in the cell therapy product is about 1:0.65.

In some embodiments, the final ratio of CD4+ cells to CD8+ cells in the cell therapy product is about 1:0.70.

In some embodiments, the final ratio of CD4+ cells to CD8+ cells in the cell therapy product is about 1:0.75.

In some embodiments, the final ratio of CD4+ cells to CD8+ cells in the cell therapy product is about 1:0.80.

In some embodiments, the final ratio of CD4+ cells to CD8+ cells in the cell therapy product is about 1:0.85.

In some embodiments, the final ratio of CD4+ cells to CD8+ cells in the cell therapy product is about 1:0.90.

In some embodiments, the manipulation comprises preparing the cell therapy product with TCM cells (CD45RO+CCR7+CD95+) and/or TEM cells (CD45RO+CCR7−CD95+).

In some embodiments, the final percentage of TCM cells in the cell therapy product is at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%.

In some embodiments, the final percentage of TEM cells in the cell therapy product is at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%.

In some embodiments, the final percentage of TCM cells in the cell therapy product is at least about 30%.

In some embodiments, the final percentage of TCM cells in the cell therapy product is at least about 35%.

In some embodiments, the final percentage of TCM cells in the cell therapy product is at least about 40%.

In some embodiments, the final percentage of TCM cells in the cell therapy product is at least about 45%.

In some embodiments, the final percentage of TCM cells in the cell therapy product is at least about 50%.

In some embodiments, the final percentage of TEM cells in the cell therapy product is at least about 50%.

In some embodiments, the final percentage of TEM cells in the cell therapy product is at least about 55%.

In some embodiments, the final percentage of TEM cells in the cell therapy product is at least about 60%.

In some embodiments, the final percentage of TEM cells in the cell therapy product is at least about 65%.

In some embodiments, the final percentage of TEM cells in the cell therapy product is at least about 70%.

In some embodiments, the manipulation comprises modifying the genome of the isolated cells by inactivating or disrupting the TRAC gene locus.

In some embodiments, the inactivation or disruption comprises inactivation or disruption of one TRAC allele or both TRAC alleles.

In some embodiments, the TRAC inactivation or disruption efficiency is at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the TRAC inactivation or disruption efficiency is at least about 95%.

In some embodiments, the TRAC inactivation or disruption efficiency is at least about 96%.

In some embodiments, the TRAC inactivation or disruption efficiency is at least about 97%.

In some embodiments, the TRAC inactivation or disruption efficiency is at least about 98%.

In some embodiments, the TRAC inactivation or disruption efficiency is at least about 99%.

In some embodiments, the manipulation comprises modifying the genome of the isolated cells by inactivating or disrupting the B2M gene locus.

In some embodiments, the inactivation or disruption comprises inactivation or disruption of one B2M allele or both B2M alleles.

In some embodiments, the B2M inactivation or disruption efficiency is at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the B2M inactivation or disruption efficiency is at least about 87%.

In some embodiments, the B2M inactivation or disruption efficiency is at least about 88%.

In some embodiments, the B2M inactivation or disruption efficiency is at least about 89%.

In some embodiments, the B2M inactivation or disruption efficiency is at least about 90%.

In some embodiments, the B2M inactivation or disruption efficiency is at least about 91%.

In some embodiments, the manipulation comprises modifying the genome of the isolated cells by inactivating or disrupting the CIITA gene locus.

In some embodiments, the inactivation or disruption comprises inactivation or disruption of one CIITA allele or both CIITA alleles.

In some embodiments, the CIITA inactivation or disruption efficiency is at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the CIITA inactivation or disruption efficiency is at least about 90%.

In some embodiments, the CIITA inactivation or disruption efficiency is at least about 91%.

In some embodiments, the CIITA inactivation or disruption efficiency is at least about 92%.

In some embodiments, the CIITA inactivation or disruption efficiency is at least about 93%.

In some embodiments, the CIITA inactivation or disruption efficiency is at least about 94%.

In some embodiments, the CIITA inactivation or disruption efficiency is at least about 95%.

In some embodiments, the CIITA inactivation or disruption efficiency is at least about 96%.

In some embodiments, the CIITA inactivation or disruption efficiency is at least about 97%.

In some embodiments, inactivation or disruption of a gene locus is measured by cell surface expression.

In some embodiments, the method further comprises selecting the donor or population of donors according to a method as described herein.

In some embodiments, the isolated cells are selected from the group consisting of islet cells, beta islet cells, pancreatic islet cells, immune cells, B cells, T cells, natural killer (NK) cells, natural killer T (NKT) cells, macrophages, endothelial cells, muscle cells, cardiac muscle cells, smooth muscle cells, skeletal muscle cells, dopaminergic neurons, retinal pigmented epithelium cells (e.g., retinal pigmented epithelium (RPE) cells and thyroid cells), optic cells, hepatocytes, thyroid cells, skin cells, glial progenitor cells, neural cells (e.g., cerebral endothelial cells, dopaminergic neurons, glial cells, and hematopoietic stem cells (HSCS) cells), cardiac cells, stem cells, hematopoietic stem cells, induced pluripotent stem cells (iPSCs), mesenchymal stem cells (MSCs), embryonic stem cells (ESCs), pluripotent stem cells (PSCs), or blood cells

In some embodiments, the isolated cells are T cells.

In some embodiments, the T cells comprises a CAR.

In some embodiments, the T cells comprise CAR-T cells as described herein.

In one aspect, the present disclosure provides a cell or a population of cells as described herein.

In one aspect, the present disclosure provides a composition containing the cell or the population of cells as described herein.

In one aspect, the disclosure is directed to a CAR-T cell therapy product as described herein below:

In this aspect, a method of making a CAR-T cell therapy product is provided, said method comprising providing a T cell or a population of T cells that have been evaluated, profiled, identified or selected according to a method as described herein and manufacturing a CAR-T cell therapy product therefrom.

In some embodiments, the method comprises:
a. isolating cells from a donor or population of donors; and
b. manipulating the isolated cells to produce a CAR-T cell therapy product.

In some embodiments, the method further comprises cryopreserving the CAR-T cell therapy product, and cell viability of the CAR-T cell therapy product after cryopreservation is at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.

In some embodiments, the cell viability of the CAR-T cell therapy product after cryopreservation is at least about 80%.

In some embodiments, the cell viability of the CAR-T cell therapy product after cryopreservation is at least about 90%.

In some embodiments, the cell viability of the CAR-T cell therapy product after cryopreservation is at least about 95%.

In some embodiments, the manipulation comprises transducing the isolated cells with a viral vector, and transduction efficiency of the isolated cells is at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.

In some embodiments, transduction efficiency of the isolated cells is at least about 40%.

In some embodiments, transduction efficiency of the isolated cells is at least about 45%.

In some embodiments, transduction efficiency of the isolated cells is at least about 50%.

In some embodiments, transduction efficiency of the isolated cells is at least about 55%.

In some embodiments, transduction efficiency of the isolated cells is at least about 60%.

In some embodiments, transduction efficiency of the isolated cells is at least about 65%.

In some embodiments, the manipulation comprises transducing the isolated cells with a viral vector, and viral copy number (VCN) in the transduced cells is no more than about 5.0, no more than about 4.7, no more than about 4.5, no more than about 4.2, no more than about 4.0, no more than about 3.7, no more than about 3.5, no more than about 3.2, no more than about 3.0, no more than about 2.7, no more than about 2.5, no more than about 2.2, no more than about 2, no more than about 1.7, no more than about 1.5, no more than about 1.2, no more than about 1.0, no more than about 0.7, no more than about 0.5, no more than about 0.2, or no more than about 0.1.

In some embodiments, the VCN in the transduced cells is no more than about 3.0.

In some embodiments, the VCN in the transduced cells is no more than about 2.7.

In some embodiments, the VCN in the transduced cells is no more than about 2.5.

In some embodiments, the VCN in the transduced cells is no more than about 2.2.

In some embodiments, the VCN in the transduced cells is no more than about 2.0.

In some embodiments, the manipulation comprises modifying the genome of the isolated cells by inactivating or disrupting the TRAC gene locus and/or the TRBC gene locus and selecting the genome modified cells.

In some embodiments, the inactivation or disruption comprises inactivation or disruption of:
a. one TRAC allele or both TRAC alleles, and/or
b. one TRBC allele or both TRBC alleles.

In some embodiments, the percentage of residual TCRαβ+ cells in the selected cells is no more than about 1.0%, no more than about 0.9%, no more than about 0.8%, no more than about 0.7%, no more than about 0.6%, no more than about 0.5%, no more than about 0.4%, no more than about 0.3%, no more than about 0.2%, or no more than about 0.1%.

In some embodiments, the percentage of residual TCRαβ+ cells in the selected cells is no more than about 0.5%.

In some embodiments, the percentage of residual TCRαβ+ cells in the selected cells is no more than about 0.4%.

In some embodiments, the percentage of residual TCRαβ+ cells in the selected cells is no more than about 0.3%.

In some embodiments, the percentage of residual TCRαβ+ cells in the selected cells is no more than about 0.2%.

In some embodiments, the percentage of residual TCRαβ+ cells in the selected cells is no more than about 0.1%.

In some embodiments, the manipulation comprises preparing the CAR-T cell therapy product with CD4+ cells and/or CD8+ cells.

In some embodiments, the final percentage of CD4+ cells in the CAR-T cell therapy product is at least about 20%, at least about 25%, at least about 30%, at least about 35%, at least about 40%, at least about 45%, at least about 50%, at least about 55%, at least about 60%, at least about 65%, at least about 70%, or at least about 75%.

In some embodiments, the final percentage of CD8+ cells in the CAR-T cell therapy product is at least about 20%, at least about 25%, at least about 30%, at least about 35%, at least about 40%, at least about 45%, at least about 50%, at least about 55%, at least about 60%, at least about 65%, at least about 70%, or at least about 75%.

In some embodiments, the final percentage of CD4+ cells in the CAR-T cell therapy product is at least about 30%.

In some embodiments, the final percentage of CD4+ cells in the CAR-T cell therapy product is at least about 35%.

In some embodiments, the final percentage of CD4+ cells in the CAR-T cell therapy product is at least about 40%.

In some embodiments, the final percentage of CD4+ cells in the CAR-T cell therapy product is at least about 45%.

In some embodiments, the final percentage of CD4+ cells in the CAR-T cell therapy product is at least about 50%.

In some embodiments, the final percentage of CD4+ cells in the CAR-T cell therapy product is at least about 55%.

In some embodiments, the final percentage of CD4+ cells in the CAR-T cell therapy product is at least about 60%.

In some embodiments, the final percentage of CD4+ cells in the CAR-T cell therapy product is at least about 65%.

In some embodiments, the final percentage of CD8+ cells in the CAR-T cell therapy product is at least about 30%.

In some embodiments, the final percentage of CD8+ cells in the CAR-T cell therapy product is at least about 35%.

In some embodiments, the final percentage of CD8+ cells in the CAR-T cell therapy product is at least about 40%.

In some embodiments, the final percentage of CD8+ cells in the CAR-T cell therapy product is at least about 45%.

In some embodiments, the final percentage of CD8+ cells in the CAR-T cell therapy product is at least about 50%.

In some embodiments, the final percentage of CD8 cells in the CAR-T cell therapy product is at least about 55%.

In some embodiments, the final percentage of CD8 cells in the CAR-T cell therapy product is at least about 60%.

In some embodiments, the final percentage of CD8 cells in the CAR-T cell therapy product is at least about 65%.

In some embodiments, the final percentage of CD8 cells in the CAR-T cell therapy product is at least about 70%.

In some embodiments, the final ratio of CD4+ cells to CD8+ cells in the CAR-T cell therapy product is about 0.4:1, about 0.45:1, about 0.5:1, about 0.55:1, about 0.6:1, about 0.65:1, about 0.7:1, about 0.75:1, about 0.8:1, about 0.85:1, about 0.9:1, about 0.95:1, about 1:1, about 1:0.95, about 1:0.9, about 1:0.85, about 1:0.8, about 1:0.75, about 1:0.7, about 1:0.65, about 1:0.6, about 1:0.55, about 1:0.5, about 1:0.45, or about 1:0.4.

In some embodiments, the final ratio of CD4+ cells to CD8+ cells in the CAR-T cell therapy product is about 0.45:1.

In some embodiments, the final ratio of CD4+ cells to CD8+ cells in the CAR-T cell therapy product is about 0.50:1.

In some embodiments, the final ratio of CD4+ cells to CD8+ cells in the CAR-T cell therapy product is about 0.55:1.

In some embodiments, the final ratio of CD4+ cells to CD8+ cells in the CAR-T cell therapy product is about 0.60:1.

In some embodiments, the final ratio of CD4+ cells to CD8+ cells in the CAR-T cell therapy product is about 0.65:1.

In some embodiments, the final ratio of CD4+ cells to CD8+ cells in the CAR-T cell therapy product is about 1:0.60.

In some embodiments, the final ratio of CD4+ cells to CD8+ cells in the CAR-T cell therapy product is about 1:0.65.

In some embodiments, the final ratio of CD4+ cells to CD8+ cells in the CAR-T cell therapy product is about 1:0.70.

In some embodiments, the final ratio of CD4+ cells to CD8+ cells in the CAR-T cell therapy product is about 1:0.75.

In some embodiments, the final ratio of CD4+ cells to CD8+ cells in the CAR-T cell therapy product is about 1:0.80.

In some embodiments, the final ratio of CD4+ cells to CD8+ cells in the CAR-T cell therapy product is about 1:0.85.

In some embodiments, the final ratio of CD4+ cells to CD8+ cells in the CAR-T cell therapy product is about 1:0.90.

In some embodiments, the manipulation comprises preparing the CAR-T cell therapy product with TCM cells (CD45RO+CCR7+CD95+) and/or TEM cells (CD45RO+CCR7−CD95+).

In some embodiments, the final percentage of TCM cells in the CAR-T cell therapy product is at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%.

In some embodiments, the final percentage of TEM cells in the CAR-T cell therapy product is at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%.

In some embodiments, the final percentage of TCM cells in the CAR-T cell therapy product is at least about 30%.

In some embodiments, the final percentage of TCM cells in the CAR-T cell therapy product is at least about 35%.

In some embodiments, the final percentage of TCM cells in the CAR-T cell therapy product is at least about 40%.

In some embodiments, the final percentage of TCM cells in the CAR-T cell therapy product is at least about 45%.

In some embodiments, the final percentage of TCM cells in the CAR-T cell therapy product is at least about 50%.

In some embodiments, the final percentage of TEM cells in the CAR-T cell therapy product is at least about 50%.

In some embodiments, the final percentage of TEM cells in the CAR-T cell therapy product is at least about 55%.

In some embodiments, the final percentage of TEM cells in the CAR-T cell therapy product is at least about 60%.

In some embodiments, the final percentage of TEM cells in the CAR-T cell therapy product is at least about 65%.

In some embodiments, the final percentage of TEM cells in the CAR-T cell therapy product is at least about 70%.

In some embodiments, the manipulation comprises modifying the genome of the isolated cells by inactivating or disrupting the TRAC gene locus.

In some embodiments, the inactivation or disruption comprises inactivation or disruption of one TRAC allele or both TRAC alleles.

In some embodiments, the TRAC inactivation or disruption efficiency is at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the TRAC inactivation or disruption efficiency is at least about 95%.

In some embodiments, the TRAC inactivation or disruption efficiency is at least about 96%.

In some embodiments, the TRAC inactivation or disruption efficiency is at least about 97%.

In some embodiments, the TRAC inactivation or disruption efficiency is at least about 98%.

In some embodiments, the TRAC inactivation or disruption efficiency is at least about 99%.

In some embodiments, the manipulation comprises modifying the genome of the isolated cells by inactivating or disrupting the B2M gene locus.

In some embodiments, the inactivation or disruption comprises inactivation or disruption of one B2M allele or both B2M alleles.

In some embodiments, the B2M inactivation or disruption efficiency is at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the B2M inactivation or disruption efficiency is at least about 87%.

In some embodiments, the B2M inactivation or disruption efficiency is at least about 88%.

In some embodiments, the B2M inactivation or disruption efficiency is at least about 89%.

In some embodiments, the B2M inactivation or disruption efficiency is at least about 90%.

In some embodiments, the B2M inactivation or disruption efficiency is at least about 91%.

In some embodiments, the manipulation comprises modifying the genome of the isolated cells by inactivating or disrupting the CIITA gene locus.

In some embodiments, the inactivation or disruption comprises inactivation or disruption of one CIITA allele or both CIITA alleles.

In some embodiments, the CIITA inactivation or disruption efficiency is at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the CIITA inactivation or disruption efficiency is at least about 90%.

In some embodiments, the CIITA inactivation or disruption efficiency is at least about 91%.

In some embodiments, the CIITA inactivation or disruption efficiency is at least about 92%.

In some embodiments, the CIITA inactivation or disruption efficiency is at least about 93%.

In some embodiments, the CIITA inactivation or disruption efficiency is at least about 94%.

In some embodiments, the CIITA inactivation or disruption efficiency is at least about 95%.

In some embodiments, the CIITA inactivation or disruption efficiency is at least about 96%.

In some embodiments, the CIITA inactivation or disruption efficiency is at least about 97%.

In some embodiments, inactivation or disruption of a gene locus is measured by cell surface expression.

In some embodiments, the method further comprises selecting the donor or population of donors according to a method as described herein.

In some embodiments, the isolated cells are selected from the group consisting of islet cells, beta islet cells, pancreatic islet cells, immune cells, B cells, T cells, natural killer (NK) cells, natural killer T (NKT) cells, macrophages, endothelial cells, muscle cells, cardiac muscle cells, smooth muscle cells, skeletal muscle cells, dopaminergic neurons, retinal pigmented epithelium cells (e.g., retinal pigmented epithelium (RPE) cells and thyroid cells), optic cells, hepatocytes, thyroid cells, skin cells, glial progenitor cells, neural cells (e.g., cerebral endothelial cells, dopaminergic neurons, glial cells, and hematopoietic stem cells (HSCS) cells), cardiac cells, stem cells, hematopoietic stem cells, induced pluripotent stem cells (iPSCs), mesenchymal stem cells (MSCs), embryonic stem cells (ESCs), pluripotent stem cells (PSCs), or blood cells

In some embodiments, the isolated cells are T cells.

In some embodiments, the T cells comprises a CAR.

In some embodiments, the T cells comprise CAR-T cells as described herein.

In one aspect, the present disclosure provides a cell or a population of cells as described herein.

In one aspect, the present disclosure provides a composition containing the cell or the population of cells as described herein.

In the methods disclosed herein, for any cell feature described herein, the method may comprise using the presence or absence of said feature as a parameter for assessing donor capability of the cell. The method may further comprise determining the presence or absence of said feature and using that determination as a parameter for assessing donor capability of the cell.

### I. Assessing Donor Capability

The present disclosure is based, at least in part, on the surprising and unexpected discovery that despite phenotypic similarities, cells have characteristics that impact donor functionality. A group of assays have been identified that can inform on donor-to-donor variability, allowing profiling of cells or populations of cells. Assays assessing cell parameters provide a read out of donor cell quality and correlate with cell function. The present disclosure therefore relates to methods for profiling a population of cells for donor capability. Any of the assays, including transcriptional profiling described herein, either alone or in combination can be used for identifying cells suitable for administration to a patient as a cell therapy or for making a cell therapy product.

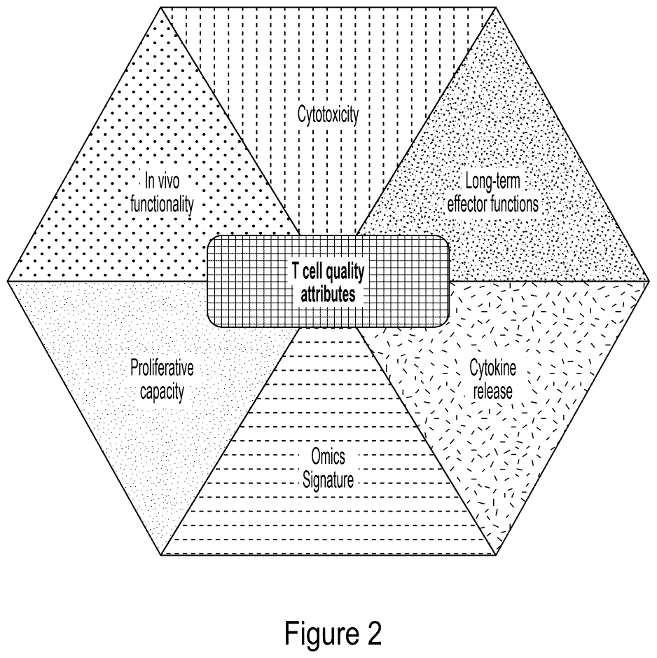

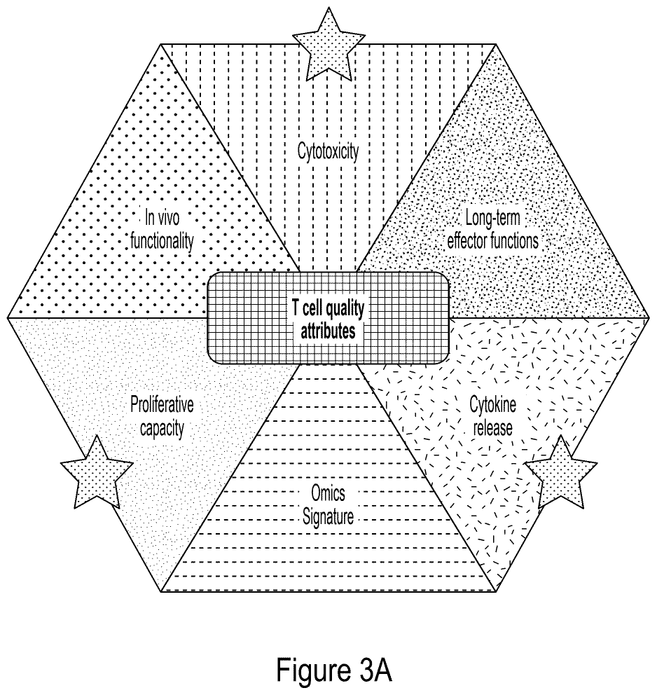

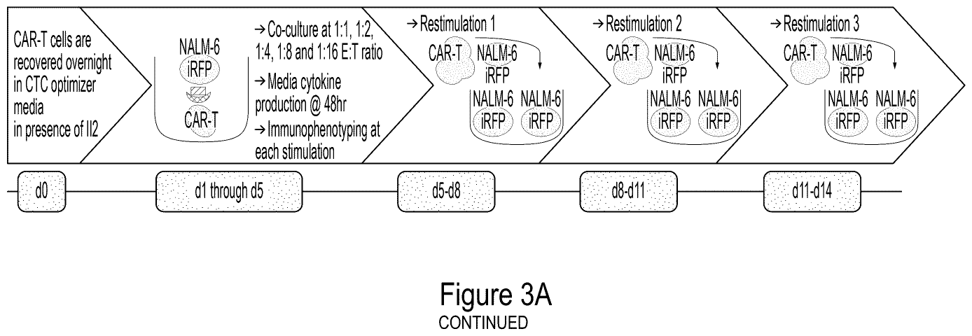

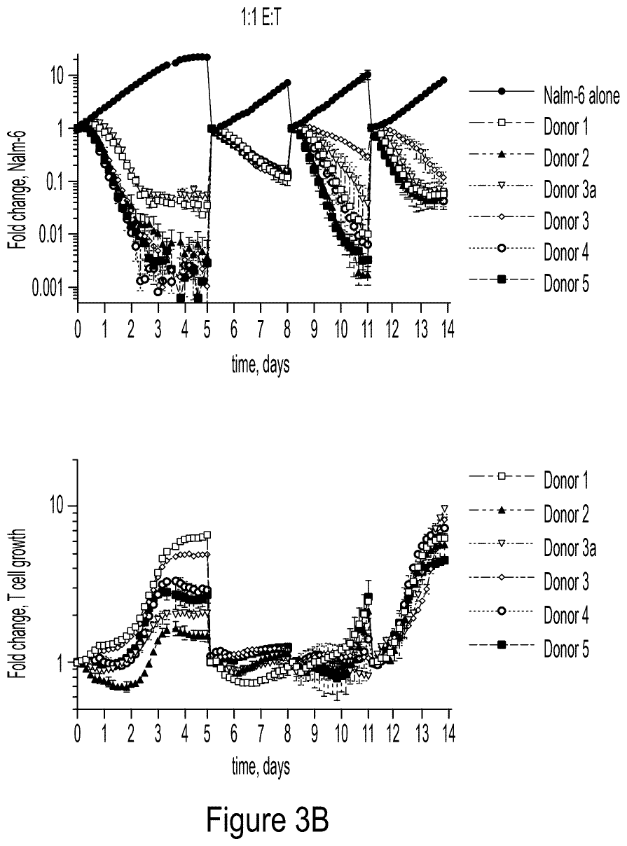

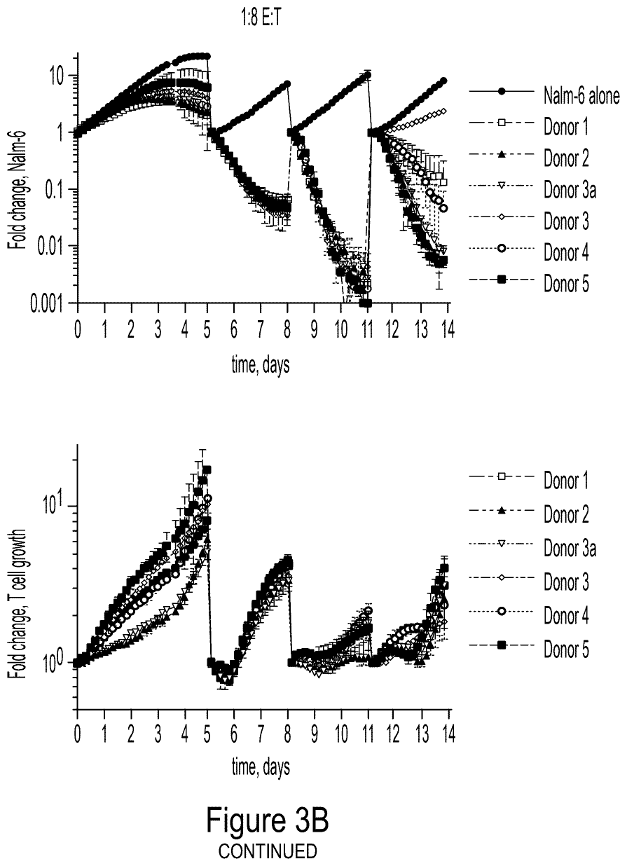

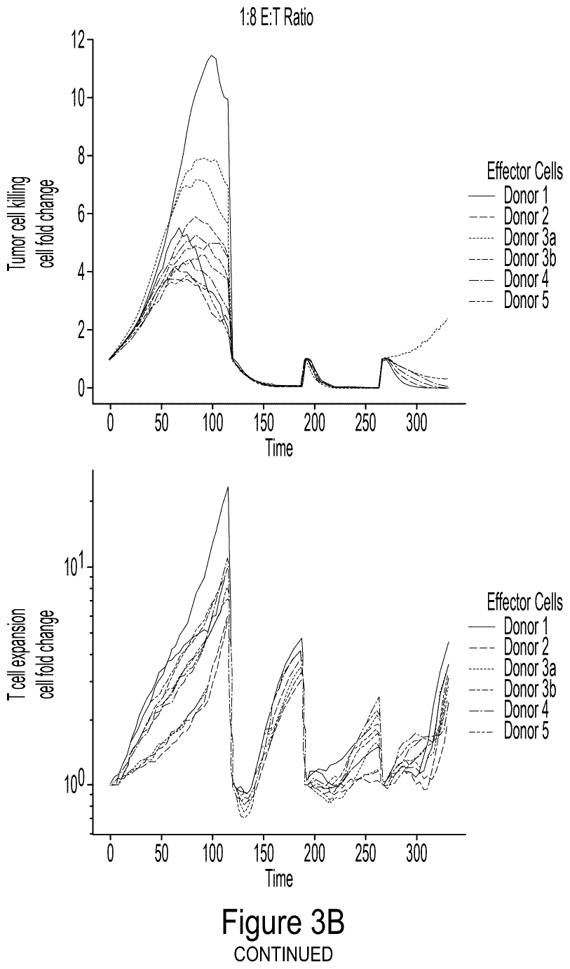

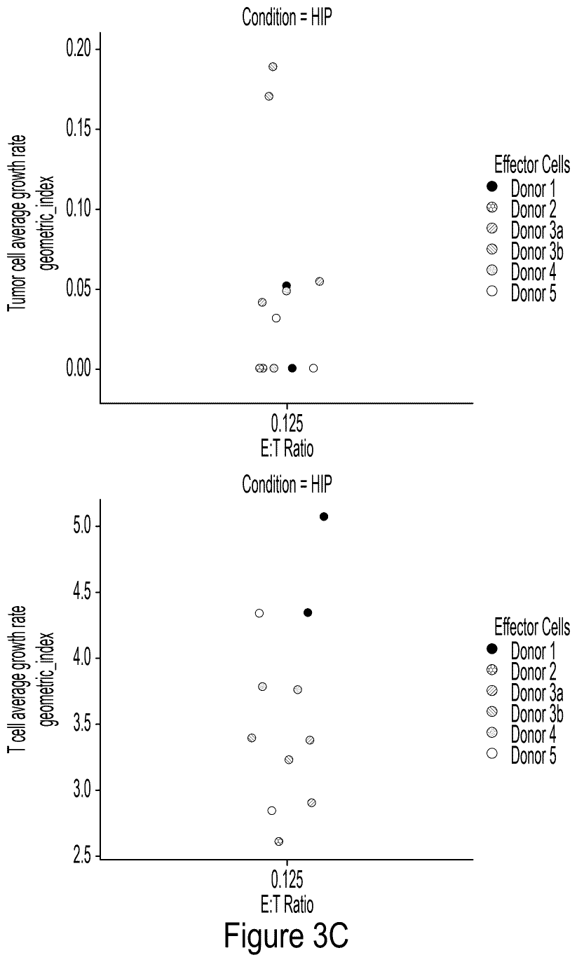

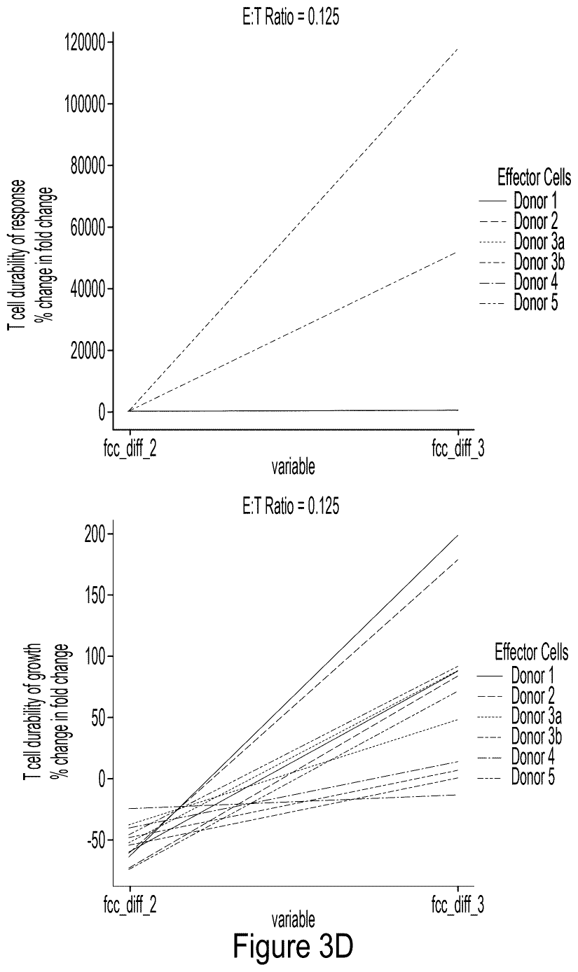

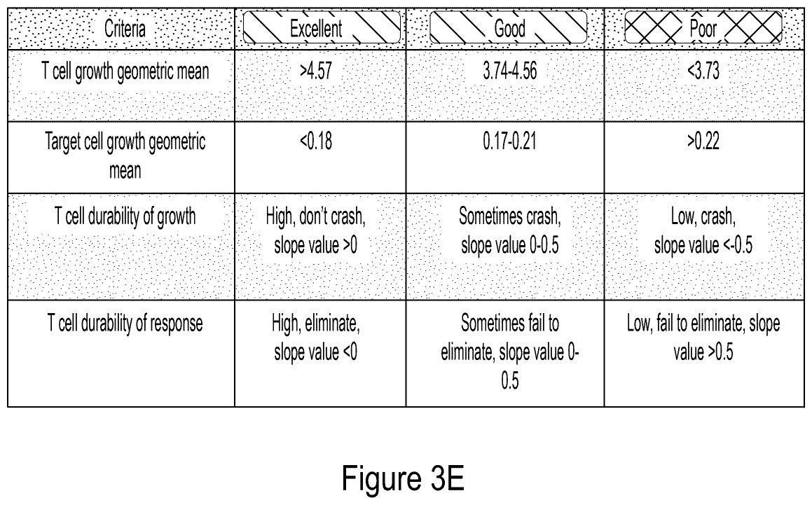

Donor capability is assessed on the basis of cell parameters. For example, the cell parameters assessed by the methods described herein are one or more of cytotoxicity, long-term effector functions, cytokine release, in vivo functionality, proliferative capacity and omnics signature (see FIG. 2).

Once read-outs have been obtained from the assays, including transcriptional profiling described herein, used either alone or in combination, any suitable means may be used to enable identification of cells suitable for administration to a patient as a cell therapy. In this regard, a number of exemplary approaches are described herein for assessing donor capability including single value cut-offs, as well as profiling scales. In some instances, assessing donor capability can be on the basis of categorising using a scale of single values (e.g., single number values).

Methods of profiling described herein allow categorization of cells e.g., into ‘exceptional’, ‘good’, or ‘poor’-performing donors. Methods of profiling described herein allow categorization of cells e.g., into ‘useable’ or ‘not useable’ donors. These assays provide single, quantitative readouts on cell quality when cells are placed under ‘stress’ and are used to predictively measure quality differences between healthy donors. These assays, either alone or in combination, can distinguish donors and thus be used for screening candidate populations of cells for high quality donor cells.

Assays that assess differences in cell performance when placed under stress include, but are not limited to, those with low effector to target (E:T) ratios or repetitive (serial) tumor challenge.

During repetitive tumor challenge (e.g., at least two, at least three, at least four or more than four tumor challenges), cell expansion may be measured in vitro during each restimulation cycle. Both cell and tumor cell (target cell) expansion may be measured. Cell expansion may be measure over a time period such as 7, 8, 9, 10, 11, 12, 13, 14, 15, 16, 17, 18, 19, 20, 21 days, and in embodiments, 14 days. The assay evaluates several aspects of cell functionality, including serial killing ability, long term cell expansion, and cell exhaustion. Assays for assessing cell expansion include but are not limited to the IncuCyte® platform. Target cells may be seeded in plate format, and cells may be seeded at varying E:T ratios (e.g., 1:1, 1:2, 1:4, 1:8, 1:16 E:T ratio). After initial stimulation (e.g., 3-10 days, such as a 5-day stimulation), a portion of the culture (e.g., 25%) may be transferred to the new plate containing fresh target cells for restimulation (e.g., 1-10 days, such as a 3-day restimulation). The skilled person will readily be able to determine a suitable period after stimulation based on the cell type. In some embodiments, the initial stimulation is a 5-day stimulation. In some embodiments, the initial stimulation is a 3-day stimulation. This step may be repeated at least two, at least three, at least four or more than four consecutive times. The cell and tumor cell average growth rate (proliferative index) may be quantified over each re-stimulation. The proliferative index can be calculated as the geometric mean of fold change for each stimulation cycle (e.g., at 1:8 E:T). Interpretation of the proliferation index is the average growth rate (tumor or T cell) across ‘n’ stimulations. Cell durability of growth and response may be quantified as slope of the fold change of restimulation for cell or target cell growth respectively. Success may be measured as a low tumor cell proliferation index and a higher cell proliferation index through the assay (assuming a reverse correlation between tumor cell and T cell growth in assay). Better performing cells correlate with inhibition of tumor cell growth and a positive expansion of cells. The skilled person will readily be able to determine a desired growth rate, and this may be predetermined (e.g., based on cell type). In embodiments, better performing cells may have an average growth rate of at least 4-fold during each restimulation cycle (e.g., compared to an average target cell growth rate of 0.2-fold). Exceptional donors may have an expansion index at least 10% above the median. Average durability of cell growth may be −1, while more durable donors may show increased durability at 0 or higher. Average durability of response may be 1, with better performing cells having value of 0 or less.

Cytokine production may also be used as a readout for cell quality (e.g., bulk cytokine analysis and/or single cell cytokine production).

Bulk cytokine production may be evaluated for polyfunctionality. Assays for assessing bulk cytokine measurements include, but are not limited to, ELISA and electrochemiluminescence (ECL) methods, for example, MesoScale Discovery (MSD) analysis. Bulk cytokine analysis may include the analysis of at least 5, 6, 7, 8, 9, 10, 11, 12, 13, 14, 15, 16, 17, 18, 19, or 20 cytokines, and in embodiments, 10 cytokines. The production of four groups of cytokines may be evaluated (e.g., effector, stimulatory, regulatory and inflammatory cytokines). Example cytokines to be measured include, but are not limited to, GM-CSF, GzmA, GzmB, IFNg, TNFa, Il2, Il6, Il17A, Il1b, Il1RA. Target cells may be seeded in plate format (e.g., % flat-well. e.g., at 10k cells per well), and cells may be seeded at varying E:T ratios (e.g., 1:1, 1:2, 1:4, 1:8, 1:16 E:T ratio). Co-culture supernatant may be collected after initial seeding (e.g., at 48 hours). Cytokine production may be quantified as production per cell(pg/cell) (e.g., at 1:2 E:T). Bulk cytokine measurement may be an average of 1.5 pg cytokine production per cell. Better performing cells may produce high levels of cytokines. Exceptional donors may produce 1.9 pg/cell or higher (25% or more above average).

Single cell cytokine production may be evaluated for polyfunctionality. Assays for single cell cytokine profiling include, but are not limited to, IsoPlexis Human Adaptive Immune assay. Cells may be co-cultured with target cells. Cells may be collected after co-culture (e.g., after 48 hours) and loaded on a chip for analysis (e.g., the IsoCode chips as suggested per manufacturer). Cells may be T cells and CD4 and CD8 T cells may be isolated via positive selection (e.g., staining for CD4 or CD8) before loading onto a chip for analysis. Two metrics may be assessed: a polyfunctionality strength index (PSI) and/or a multi-functionality index. Polyfunctionality strength index may be defined as signal intensity of the cytokine production by a cell population that produces 2+ cytokines. Multi-functionality index may be defined as the percentage of cells that produce 4+ cytokines. Better performing cells may have a diverse cytokine profile produced per cell. Exceptional donors may have a PSI of greater than 15% above the median. A median PSI value of 200 may be observed for a CD8 T cell population, and 90 for a CD4 T cell population. Multifunctionality of 0.3-3% may be observed in CD8 T cell population, and 0.1-2% in CD4 T cell population.

In vivo efficacy may be evaluated using an animal tumor model. Assays for assessing in vivo efficacy include, but are not limited to, NALM-6 challenge in immune-deficient NSG mice. Tumor cells may be injected first, followed by cell injection. Injection of tumor cells may be 1, 2, 3, 4, 5, 6 or 7 days, and in embodiments, 3 days, before cell injection. Efficacy of response may be evaluated at high (e.g., 5e+6) and/or low (e.g., 0.5e+6) cell doses. In vivo response maybe calculated as area under the curve (AUC) of the target cell bioluminescent flux. Better performing cells may have an AUC of less than 20 or between 20 and 100 at high dose, or less than 7911 or between 7911-13185 at low dose. Exceptional donors may have an AUC of less than 20 at high dose, or less than 7911 at low dose.

Gene expression profiling may be used to identify distinct gene expression profiles that correlate with functional performance. Gene expression profiling may be evaluated at different timepoints e.g., any or all of the following time points: pre-production (pre-modification), post-manufacturing (post-modification or resting cells), and post-serial tumor challenge (activated and exhausted cells). Assays for gene expression profiling include, but are not limited to, using the nCounter panel (e.g., the nCounter Sprint profiler (NanoString Technologies)). For the pre-production and post-manufacturing resting conditions, the cells may be thawed overnight in presence of 112. Total mRNA may be isolated in triplicates. To generate early activated and exhausted cell subsets, cells, and target cells (e.g., Naml-6) may be seeded in plate format at varying E:T ratios (e.g., 1:1. 1:2, 1:4, 1:8. 1:16 E:T ratio, and in embodiments 1:8). Serial restimulation may be performed at least two, at least three, at least four or more than four consecutive times. Merely by way of example, the cultures may be incubated for 5 day and three restimulations performed on d5, d8 and dl1. Co-cultured cells may be isolated 2 days after initial seeding for the early activation timepoint. Co-cultured cells at the end of the assay (e.g., 14 days) represent exhausted cell timepoint. Cells may be T cells and gene expression profiling may be carried out at any or all of the above timepoints for one or both of CD4 and CD8 T cells. CD4 and CD8 cells may be isolated at any time point via positive selection.

Better performing donors may be less activated and have a less NK-like signature at the pre-production state. Additionally, better performing donors have an activated phenotype at resting cell state. Better performing donors show higher amount of mucosal associated invariant T (MAIT) cells at the pre-production, resting, and early activation steps. Exceptional cells skew towards Th1/Tc1 and Th17/Tc17 states at the resting and activated stages.

Metrics from any or all of the assays described herein can be used to rank donors and correlate with in vivo success. As such, methods for profiling a population of cells for donor capability according to the present disclosure may advantageously form a part of an overall method for manufacturing a cell therapy product.

Any reference of a method or assay herein should not be interpreted as limited to a single method or assay. Any of the assays or methods described herein can be used individually or in combination within the methods described herein.

It will be understood that, in the process of manufacturing a cell therapy, certain modifications may be introduced to the cell that are considered desirable for the cell therapy product. Cells that are profiled for donor capability can be edited or unedited cells. Profiling cells can take place before or after cell editing. Edited cells include one or more modifications such as HIP modifications (hypoimmune gene modifications that enable immune evasion). When transplanted in vivo without immunosuppression. HIP-modified cells have reduced expression of one or more molecules of the MHC class I and/or MHC class II molecules and thus there may be no evidence of a systemic immune response, such as no T cell activation, antibody production, or NK cell activity. Disclosure relating to edited cells is provided herein. Such disclosure is applicable to the methods, uses and cells described herein. The methods for profiling a population of cells for donor capability disclosed herein are advantageous for many cell types as described herein. In some embodiments, the cells are T cells (e.g., CAR-T cells). Engineered Cells and Methods of Engineering Cells

As set out above, methods for profiling a population of cells for donor capability according to the present disclosure may advantageously form a part of an overall method for manufacturing a cell therapy product. It will be understood that, in the process of manufacturing a cell therapy, certain modifications may be introduced to the cell that are considered desirable for the cell therapy product. Cells that are profiled for donor capability can be edited or unedited cells depending on the stage of the manufacturing process at which they are profiled.

One modification considered desirable for the cell therapy product is that the engineered cells and populations thereof exhibit reduced expression of one or more molecules of the MHC class I and/or MHC class II molecules. A further modification considered desirable for the cell therapy product is that the engineered cells and populations thereof exhibit increased expression of at least one tolerogenic factor, such as tolerogenic factors described herein.

Provided herein are methods and compositions for alleviating and/or evading the effects of immune system reactions to allogenic transplants. To overcome the problem of immune rejection of cell-derived and/or tissue transplants, disclosed herein is an engineered immune-evasive cell (e.g., an engineered primary hypo-immunogenic cell), or population or pharmaceutical composition thereof, that represents a viable source for any transplantable cell type. The engineered cells disclosed herein provide for reduced recognition the recipient subject's immune system, regardless of the subject's genetic make-up, or any existing response within the subject to one or more previous allogeneic transplants, previous autologous chimeric antigen receptor (CAR) T rejection, and/or other autologous or allogenic therapies wherein a transgene is expressed. The engineered cells may include, but are not limited to, beta islet cells, B cells, T cells, NK cells, retinal pigmented epithelium cells, glial progenitor cells, endothelial cells, hepatocytes, thyroid cells, skin cells, and blood cells (e.g., plasma cells or platelets).

In some embodiments, the engineered cells described herein further comprise increased expression and/or overexpression of one or more complement inhibitors. In some embodiments, the one or more complement inhibitors are selected from CD46, CD59, and CD55. In some embodiments, the engineered cells comprise increased expression of two or more complement inhibitors in combination, such as increased expression of CD46 and CD59 or increased expression of CD46, CD59, and CD55.

The engineered cells provided herein utilize expression of tolerogenic factors and can also modulate (e.g., reduce or eliminate) one or more MHC class I molecules and/or one or more MHC class II molecules expression (e.g., surface expression). In some embodiments, genome editing technologies utilizing rare-cutting endonucleases (e.g., the CRISPR/Cas, TALEN, zinc finger nuclease, meganuclease, and homing endonuclease systems) are also used to reduce or eliminate expression of critical immune genes (e.g., by deleting genomic DNA of critical immune genes) in human cells. In certain embodiments, genome editing technologies or other gene modulation technologies are used to insert tolerance-inducing (tolerogenic) factors in human cells, (e.g., CD47), thus producing engineered cells that can evade immune recognition upon engrafting into a recipient subject. Therefore, the engineered cells provided herein exhibit modulated expression (e.g., reduced or eliminated expression) of one or more genes and factors that affect one or more MHC class I molecules and/or one or more MHC class II molecules, modulated expression (e.g., reduced or and modulated expression (e.g., overexpression) of tolerogenic factors, such as CD47, and provide for reduced recognition by the recipient subject's immune system. In some embodiments, the engineered cells provided herein exhibit modulated expression (e.g., reduced expression) of CD142. In some embodiments, the engineered cells provided herein exhibit modulated expression (e.g., increased expression) of one or more complement inhibitors selected from CD46. CD59, and CD55.

In some aspects, engineered cells provided herein exhibit reduced innate immune cell rejection and/or adaptive immune cell rejection (e.g., hypo-immunogenic cells). For example, in some embodiments, the engineered cells exhibit reduced susceptibility to NK cell-mediated lysis and/or macrophage engulfment. In some embodiments, the engineered cells are useful as a source of universally compatible cells or tissues (e.g., universal donor cells or tissues) that are transplanted into a recipient subject with little to no immunosuppressant agent needed. Such hypo-immunogenic cells retain cell-specific characteristics and features upon transplantation.

Also provided herein are methods for treating a disorder comprising administering the engineered cells (e.g., engineered primary cells) that evade immune rejection in an MHC-mismatched allogenic recipient. In some embodiments, the engineered cells produced from any one of the methods described herein evade immune rejection when repeatedly administered (e.g., transplanted or grafted) to MHC-mismatched allogenic recipient.

The practice of the embodiments described herein will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); Anand, Techniques for the Analysis of Complex Genomes, (Academic Press, New York. 1992); Transcription and Translation (B. Hames & S. Higgins, Eds., 1984); Perbal, A Practical Guide to Molecular Cloning (1984); Harlow and Lane, Antibodies, (Cold Spring Harbor Laboratory Press, Cold Spring Harbor, N.Y., 1998) Current Protocols in Immunology Q. E. Coligan, A. M. Kruisbeek, D. H. Margulies, E. M. Shevach and W. Strober, eds., 1991); Annual Review of Immunology; as well as monographs in journals such as Advances in Immunology.

All publications, including patent documents, scientific articles, and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described. Those skilled in the art will recognize that several embodiments are possible within the scope and spirit of the present disclosure. The following description illustrates the disclosure and, of course, should not be construed in any way as limiting the scope of contemplated embodiments described herein.

Described here are engineered cells that comprise one or more modifications. In some embodiments, the provided engineered cells also contain a modification of one or more target polynucleotide sequences that regulates the expression of one or more MHC class I molecules, one or more MHC class II molecules, or one or more MHC class I molecules and one or more MHC class II molecules.

In some embodiments, the provided engineered cells also include a modification to increase expression of one or more tolerogenic factor. In some embodiments, the tolerogenic factor is one or more of CD16, CD24, CD35, CD39. CD46. CD47, CD52, CD55, CD59, CD200, CCL22, CTLA4-Ig, C1 inhibitor, FASL, IDO1, HLA-C, HLA-E, HLA-E heavy chain. HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, MANF, or any combination thereof. In some embodiments, the modification to increase expression of one or more tolerogenic factor is or includes increased expression of CD47. In some embodiments, the modification to increase expression of one or more tolerogenic factor is or includes increased expression of PD-L1. In some embodiments, the modification to increase expression of one or more tolerogenic factor is or includes increased expression of HLA-E. In some embodiments, the modification to increase expression of one or more tolerogenic factor is or includes increased expression of HLA-G. In some embodiments, the modification to increase expression of one or more tolerogenic factor is or includes increased expression of CCL21, PD-L1, FasL, Serpinb9, H2-M3 (HLA-G), CD47, CD200, and Mfge8.

In some embodiments, the cells include one or more genomic modifications that reduce expression of one or more MHC class I molecules and a modification that increases expression of CD47. In other words, the engineered cells comprise exogenous CD47 proteins and exhibit reduced or silenced surface expression of one or more MHC class I molecules. In some embodiments, the cells include one or more genomic modifications that reduce expression of one or more MHC class II molecules and a modification that increases expression of CD47. In some instances, the engineered cells comprise exogenous CD47 nucleic acids and proteins, and exhibit reduced or silenced surface expression of one or more MHC class I molecules. In some embodiments, the cells include one or more genomic modifications that reduce or eliminate expression of one or more MHC class II molecules, one or more genomic modifications that reduce or eliminate expression of one or more MHC class II molecules, and a modification that increases expression of CD47. In some embodiments, the engineered cells comprise exogenous CD47 proteins, exhibit reduced or silenced surface expression of one or more MHC class I molecules and exhibit reduced or lack surface expression of one or more MHC class II molecules. In many embodiments, the cells are B2M^{indel/indel}, CIITA^{indel/indel}, CD47tg cells.

In some embodiments, any of gene editing technologies can be used to reduce expression of the one or more target polynucleotides or target proteins as described. In some embodiments, the gene editing technology can include systems involving nucleases, integrases, transposases, recombinases. In some embodiments, the gene editing technologies can be used for knock-out or knock-down of genes. In some embodiments, the gene-editing technologies can be used for knock-in or integration of DNA into a region of the genome. In some embodiments, the gene editing technology mediates single-strand breaks (SSB). In some embodiments, the gene editing technology mediates double-strand breaks (DSB), including in connection with non-homologous end-joining (NHEJ) or homology-directed repair (HDR). In some embodiments, the gene editing technology can include DNA-based editing or prime-editing. In some embodiments, the gene editing technology can include Programmable Addition via Site-specific Targeting Elements (PASTE).

In some embodiments, the gene editing technology is associated with base editing. Base editors (BEs) are typically fusions of a Cas (“CRISPR-associated”) domain and a nucleobase modification domain (e.g., a natural or evolved deaminase, such as a cytidine deaminase that include APOBEC1 (“apolipoprotein B mRNA editing enzyme, catalytic polypeptide 1”), CDA (“cytidine deaminase”), and AID (“activation-induced cytidine deaminase”)) domains. In some cases, base editors may also include proteins or domains that alter cellular DNA repair processes to increase the efficiency and/or stability of the resulting single-nucleotide change.

In some aspects, currently available base editors include cytidine base editors (e.g., BE4) that convert target C•G to T•A and adenine base editors (e.g., ABE7.10) that convert target A•T to G•C. In some aspects. Cas9-targeted deamination was first demonstrated in connection with a Base Editor (BE) system designed to induce base changes without introducing double-strand DNA breaks. Further Rat deaminase APOBEC1 (rAPOBEC1) fused to deactivated Cas9 (dCas9) was used to successfully convert cytidines to thymidines upstream of the PAM of the sgRNA. In some aspects, this first BE system was optimized by changing the dCas9 to a “nickase” Cas9 D10A, which nicks the strand opposite the deaminated cytidine. Without being bound by theory, this is expected to initiate long-patch base excision repair (BER), where the deaminated strand is preferentially used to template the repair to produce a U:A base pair, which is then converted to T:A during DNA replication.

In some embodiments, the base editor is a nucleobase editor containing a first DNA binding protein domain that is catalytically inactive, a domain having base editing activity, and a second DNA binding protein domain having nickase activity, where the DNA binding protein domains are expressed on a single fusion protein or are expressed separately (e.g., on separate expression vectors). In some embodiments, the base editor is a fusion protein comprising a domain having base editing activity (e.g., cytidine deaminase or adenosine deaminase), and two nucleic acid programmable DNA binding protein domains (napDNAbp), a first comprising nickase activity and a second napDNAbp that is catalytically inactive, wherein at least the two napDNAbp are joined by a linker. In some embodiments, the base editor is a fusion protein that comprises a DNA domain of a CRISPR-Cas (e.g., Cas9) having nickase activity (nCas; nCas9), a catalytically inactive domain of a CRISPR-Cas protein (e.g., Cas9) having nucleic acid programmable DNA binding activity (dCas; e.g., dCas9), and a deaminase domain, wherein the dCas is joined to the nCas by a linker, and the dCas is immediately adjacent to the deaminase domain. In some embodiments, the base editor is an adenine-to-thymine or “ATBE” (or thymine-to-adenine or “TABE”) transversion base editors. Exemplary base editor and base editor systems include any as described in patent publication Nos. US20220127622, US20210079366, US20200248169, US20210093667, US20210071163, WO2020181202, WO2021158921, WO2019126709, WO2020181178, WO2020181195, WO2020214842, WO2020181193, which are hereby incorporated in their entirety.

In some embodiments, the gene editing technology is target-primed reverse transcription (TPRT) or “prime editing”. In some embodiments, prime editing mediates targeted insertions, deletions, all 12 possible base-to-base conversions, and combinations thereof in human cells without requiring DSBs or donor DNA templates.

Prime editing is a genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand of the target site to be edited (with the exception that it includes the desired edit). Through DNA repair and/or replication machinery, the endogenous strand of the target site is replaced by the newly synthesized replacement strand containing the desired edit. In some cases, prime editing may be thought of as a “search-and- replace” genome editing technology since the prime editors search and locate the desired target site to be edited and encode a replacement strand containing a desired edit which is installed in place of the corresponding target site endogenous DNA strand at the same time. For example, prime editing can be adapted for conducting precision CRISPR/Cas-based genome editing in order to bypass double stranded breaks. In some embodiments, the homologous protein is or encodes for a Cas protein-reverse transcriptase fusions or related systems to target a specific DNA sequence with a guide RNA, generate a single strand nick at the target site, and use the nicked DNA as a primer for reverse transcription of an engineered reverse transcriptase template that is integrated with the guide RNA. In some embodiments, the prime editor protein is paired with two prime editing guide RNAs (pegRNAs) that template the synthesis of complementary DNA flaps on opposing strands of genomic DNA, resulting in the replacement of endogenous DNA sequence between the PE-induced nick sites with pegRNA-encoded sequences.

In some embodiments, the gene editing technology is associated with a prime editor that is a reverse transcriptase, or any DNA polymerase known in the art. Thus, in one aspect, the prime editor may comprise Cas9 (or an equivalent napDNAbp) which is programmed to target a DNA sequence by associating it with a specialized guide RNA (i.e., PEgRNA) containing a spacer sequence that anneals to a complementary protospacer in the target DNA. Such methods include any disclosed in Anzalone et al., (doi.org/10.1038/s41586-019-1711-4), or in PCT publication Nos. WO2020191248, WO2021226558, or WO2022067130, which are hereby incorporated in their entirety.

In some embodiments, the gene editing technology is Programmable Addition via Site-specific Targeting Elements (PASTE). In some aspects, PASTE is platform in which genomic insertion is directed via a CRISPR-Cas9 nickase fused to both a reverse transcriptase and serine integrase. As described in Ioannidi et al. (doi.org/10.1101/2021.11.01.466786), PASTE does not generate double stranded breaks, but allows for integration of sequences as large as ˜36 kb. In some embodiments, the serine integrase can be any known in the art. In some embodiments, the serine integrase has sufficient orthogonality such that PASTE can be used for multiplexed gene integration, simultaneously integrating at least two different genes at least two genomic loci. In some embodiments, PASTE has editing efficiencies comparable to or better than those of homology directed repair or non-homologous end joining based integration, with activity in nondividing cells and fewer detectable off-target events.

In some embodiments, CRISPR systems of the present disclosure comprise TnpB polypeptides. In some embodiments, TnpB polypeptides may comprise a Ruv-C-like domain. The RuvC domain may be a split RuvC domain comprising RuvC-I, RuvC-II, and RuvC-III subdomains. In some embodiments, a TnpB may further comprise one or more of a HTH domain, a bridge helix domain, and a zinc finger domain. TnpB polypeptides do not comprise an HNH domain. In some embodiments, a TnpB protein comprises, starting at the N-terminus: a HTH domain, a RuvC-I subdomain, a bridge helix domain, a RuvC-II sub-domain, a zinger finger domain, and a RuvC-III sub-domain. In some embodiments, a RuvC-III sub-domain forms the C-terminus of a TnpB polypeptide. In some embodiments, a TnpB polypeptide is from Epsilonproteobacteria bacterium, Actinoplanes lobatus strain DSM 43150. Actinomadura celluolosilytica strain DSM 45823, Actinomadura namibiensis strain DSM 44197, Alicyclobacillus macrosprangiidus strain DSM 17980, Lipingzhangella halophila strain DSM 102030, or Ktedonobacter racemifer. In some embodiments, a TnpB polypeptide is from Ktedonobacter racemifer, or comprises a conserved RNA region with similarity to the 5′ ITR of K. racemifer TnpB loci. In some embodiments, a TnpB may comprise a Fanzor protein, a TnpB homolog found in eukaryotic genomes. In some embodiments, a CRISPR system comprising a TnpB polypeptide binds a target adjacent motif (TAM) sequence 5′ of a target polynucleotide. In some embodiments, a TAM is a transposon-associated motif. In some embodiments, a TAM sequence comprises TCA. In some embodiments, a TAM sequence comprises TTCAN. In some embodiments, a TAM sequence comprises TTGAT. In some embodiments, a TAM sequence comprises ATAAA.

In some embodiments, the population of engineered cells described elicits a reduced level of immune activation or no immune activation upon administration to a recipient subject. In some embodiments, the cells elicit a reduced level of systemic TH1 activation or no systemic TH1 activation in a recipient subject. In some embodiments, the cells elicit a reduced level of immune activation of peripheral blood mononuclear cells (PBMCs) or no immune activation of PBMCs in a recipient subject. In some embodiments, the cells elicit a reduced level of donor-specific IgG antibodies or no donor specific IgG antibodies against the cells upon administration to a recipient subject. In some embodiments, the cells elicit a reduced level of IgM and IgG antibody production or no IgM and IgG antibody production against the cells in a recipient subject. In some embodiments, the cells elicit a reduced level of cytotoxic T cell killing of the cells upon administration to a recipient subject.

In some embodiments, the engineered cells provided herein comprise a “suicide gene” or “suicide switch”. A suicide gene or suicide switch can be incorporated to function as a “safety switch” that can cause the death of the engineered cell (e.g., primary engineered cell or cell differentiated from an engineered pluripotent stem cell), such as after the engineered cell is administered to a subject and if they cells should grow and divide in an undesired manner. The “suicide gene” ablation approach includes a suicide gene in a gene transfer vector encoding a protein that results in cell killing only when activated by a specific compound. A suicide gene may encode an enzyme that selectively converts a nontoxic compound into highly toxic metabolites. The result is specifically eliminating cells expressing the enzyme. In some embodiments, the suicide gene is the herpesvirus thymidine kinase (HSV-tk) gene, and the trigger is ganciclovir. In other embodiments, the suicide gene is the Escherichia coli cytosine deaminase (EC-CD) gene, and the trigger is 5-fluorocytosine (5-FC)(Barese et al. Mol. Therap. 20(10); 1932-1943 (2012). Xu et al, Cell Res. 8:73-8 (1998), both incorporated herein by reference in their entirety).

In other embodiments, the suicide gene is an inducible Caspase protein. An inducible Caspase protein comprises at least a portion of a Caspase protein capable of inducing apoptosis. In some embodiments, the inducible Caspase protein is iCasp9. It comprises the sequence of the human FK506-binding protein, FKBP12, with an F36V mutation, connected through a series of amino acids to the gene encoding human caspase 9. FKBP12-F36V binds with high affinity to a small-molecule dimerizing agent, API 903. Thus, the suicide function of iCasp9 is triggered by the administration of a chemical inducer of dimerization (CID). I n some embodiments, the CID is the small molecule drug API 903. Dimerization causes the rapid induction of apoptosis. (See WO2011146862; Stasi et al, N. Engl. J. Med 365; 18 (2011); Tey et al, Biol. Blood Marrow Transplant. 13:913-924 (2007), each of which are incorporated by reference herein in their entirety.)

Inclusion of a safety switch or suicide gene allows for controlled killing of the cells in the event of cytotoxicity or other negative consequences to the recipient, thus increasing the safety of cell-based therapies, including those using tolerogenic factors.

In some embodiments, a safety switch can be incorporated into, such as introduced, into the engineered cells provided herein to provide the ability to induce death or apoptosis of engineered cells containing the safety switch, for example if the cells grow and divide in an undesired manner or cause excessive toxicity to the host. Thus, the use of safety switches enables one to conditionally eliminate aberrant cells in vivo and can be a critical step for the application of cell therapies in the clinic. Safety switches and their uses thereof are described in, for example, Duzgune§, Origins of Suicide Gene Therapy (2019); Duzgune§ (eds), Suicide Gene Therapy. Methods in Molecular Biology, vol. 1895 (Humana Press, New York, NY) (for HSV-tk, cytosine deaminase, nitroreductase, purine nucleoside phosphorylase, and horseradish peroxidase): Zhou and Brenner, Exp Hematol 44(11):1013-1019 (2016) (for iCaspase9); Wang et al., Blood 18(5):1255-1263 (2001) (for huEGFR); U.S. Patent Application Publication No. 20180002397 (for HER1); and Philip et al., Blood 124(8):1277-1287 (2014) (for RQR8).

In some embodiments, the safety switch can cause cell death in a controlled manner, for example, in the presence of a drug or prodrug or upon activation by a selective exogenous compound. In some embodiments, the safety switch is selected from the group consisting of herpes simplex virus thymidine kinase (HSV-tk), cytosine deaminase (CyD), nitroreductase (NTR), purine nucleoside phosphorylase (PNP), horseradish peroxidase, inducible caspase 9 (iCasp9), rapamycin-activated caspase 9 (rapaCasp9), CCR4, CD16, CD19, CD20, CD30, EGFR, GD2, HER1, HER2, MUC1, PSMA, and RQR8.

In some embodiments, the safety switch may be a transgene encoding a product with cell killing capabilities when activated by a drug or prodrug, for example, by turning a non-toxic prodrug to a toxic metabolite inside the cell. In these embodiments, cell killing is activated by contacting an engineered cell with the drug or prodrug. In some cases, the safety switch is HSV-tk, which converts ganciclovir (GCV) to GCV-triphosphate, thereby interfering with DNA synthesis and killing dividing cells. In some cases, the safety switch is CyD or a variant thereof, which converts the antifungal drug 5-fluorocytosine (5-FC) to cytotoxic 5-fluorouracil (5-FU) by catalyzing the hydrolytic deamination of cytosine into uracil. 5-FU is further converted to potent anti-metabolites (5-FdUMP, 5-FdUTP, 5-FUTP) by cellular enzymes. These compounds inhibit thymidylate synthase and the production of RNA and DNA, resulting in cell death. In some cases, the safety switch is NTR or a variant thereof, which can act on the prodrug CB 1954 via reduction of the nitro groups to reactive N-hydroxylamine intermediates that are toxic in proliferating and nonproliferating cells. In some cases, the safety switch is PNP or a variant thereof, which can turn prodrug 6-methylpurine deoxyriboside or fludarabine into toxic metabolites to both proliferating and nonproliferating cells. In some cases, the safety switch is horseradish peroxidase or a variant thereof, which can catalyze indole-3-acetic acid (IAA) to a potent cytotoxin and thus achieve cell killing.

In some embodiments, the safety switch may be an iCasp9. Caspase 9 is a component of the intrinsic mitochondrial apoptotic pathway which, under physiological conditions, is activated by the release of cytochrome C from damaged mitochondria. Activated caspase 9 then activates caspase 3, which triggers terminal effector molecules leading to apoptosis. The iCasp9 may be generated by fusing a truncated caspase 9 (without its physiological dimerization domain or caspase activation domain) to a FK506 binding protein (FKBP), FKBP12-F36V, via a peptide linker. The iCasp9 has low dimer-independent basal activity and can be stably expressed in host cells (e.g., human T cells) without impairing their phenotype, function, or antigen specificity. However, in the presence of chemical inducer of dimerization (CID), such as rimiducid (AP1903), AP20187, and rapamycin, iCasp9 can undergo inducible dimerization and activate the downstream caspase molecules, resulting in apoptosis of cells expressing the iCasp9. See, e.g., PCT Application Publication No. WO2011/146862; Stasi et al., N. Engl. J. Med. 365;18 (2011); Tey et al., Biol. Blood Marrow Transplant 13:913-924 (2007). For example, the rapamycininducible caspase 9 variant is called rapaCasp9. See Stavrou et al., Mal. Ther. 26(5):1266-1276 (2018). Thus, iCasp9 can be used as a safety switch to achieve controlled killing of the host cells.

In some embodiments, the safety switch may be a membrane-expressed protein which allows for cell depletion after administration of a specific antibody to that protein. Safety switches of this category may include, for example, one or more transgene encoding CCR4, CD16, CD19, CD20, CD30, EGFR, GD2, HER1, HER2, MUC1, PSMA, or RQR8 for surface expression thereof. These proteins may have surface epitopes that can be targeted by specific antibodies. In some embodiments, the safety switch comprises CCR4, which can be recognized by an anti-CCR4 antibody. Non-limiting examples of suitable anti-CCR4 antibodies include mogamulizumab and biosimilars thereof. In some embodiments, the safety switch comprises CD16 or CD30, which can be recognized by an anti-CD16 or anti-CD30 antibody. Non-limiting examples of such antiCD16 or anti-CD30 antibody include AFM13 and biosimilars thereof. In some embodiments, the safety switch comprises CD19, which can be recognized by an anti-CD19 antibody. Non-limiting examples of such anti-CD19 antibody include MOR208 and biosimilars thereof. In some embodiments, the safety switch comprises CD20, which can be recognized by an anti-CD20 antibody. Non-limiting examples of such anti-CD20 antibody include obinutuzumab, ublituximab, ocaratuzumab, rituximab, rituximab-Rllb, and biosimilars thereof. Cells that express the safety switch are thus CD20-positive and can be targeted for killing through administration of an anti-CD20 antibody as described. In some embodiments, the safety switch comprises EGFR, which can be recognized by an anti-EGFR antibody. Non-limiting examples of such anti-EGFR antibody include tomuzotuximab, RO5083945 (GA201), cetuximab, and biosimilars thereof. In some embodiments, the safety switch comprises GD2, which can be recognized by an anti-GD2 antibody. Non-limiting examples of such anti-GD2 antibody include Hul4.18K322A, Hul4.18-IL2, Hu3F8, dinituximab, c.60C3-Rllc, and biosimilars thereof.

In some embodiments, the safety switch may be an exogenously administered agent that recognizes one or more tolerogenic factor on the surface of the engineered cell. In some embodiments, the exogenously administered agent is an antibody directed against or specific to a tolerogenic agent, e.g., an anti-CD47 antibody. By recognizing and blocking a tolerogenic factor on the engineered cell, an exogenously administered antibody may block the immune inhibitory functions of the tolerogenic factor thereby re-sensitizing the immune system to the engineered cells. For instance, for an engineered cell that overexpresses CD47 an exogenously administered anti-CD47 antibody may be administered to the subject, resulting in masking of CD47 on the engineered cell and triggering of an immune response to the engineered cell.

In some embodiments, provided herein is a method of generating an engineered cell, comprising: (a) reducing or eliminating the expression of one or more MHC class I molecules and/or one or more MHC class II molecules in the cell; (b) increasing the expression of a tolerogenic factor in the cell. In some embodiments, the one or more tolerogenic factor is selected from CD16, CD24, CD35, CD39, CD46, CD47, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, MANF. In some embodiments, the one or more tolerogenic factor is CD47. In some embodiments, the method comprises reducing or eliminating the expression of one or more MHC class I molecules and one or more MHC class II molecules. In some embodiments, the reducing or increasing expression comprise performing one or more modifications to the cell using a guided nuclease (e.g., a CRISPR/Cas system). In some embodiments, the method further comprises introducing an expression vector comprising an inducible suicide switch into the cell.

In some embodiments, the reducing or increasing expression comprise performing one or more modifications to the cell using a guided nuclease (e.g., a CRISPR/Cas system). In some embodiments, the method further comprises introducing an expression vector comprising an inducible suicide switch into the cell. In some embodiments, the method further comprises increasing the expression of CD55 in said cell.

In some embodiments, the tolerogenic factor is CD47 and the cell includes an exogenous polynucleotide encoding a CD47 protein. In some embodiments, the cell expresses an exogenous CD47 polypeptide.

In some embodiments, a method disclosed herein comprises administering to a subject in need thereof a CD47-SIRPα blockade agent, wherein the subject was previously administered a population of cells engineered to express an exogenous CD47 polypeptide. In some embodiments, the CD47-SIRPα blockade agent comprises a CD47-binding domain. In some embodiments, the CD47-binding domain comprises signal regulatory protein alpha (SIRPα) or a fragment thereof. In some embodiments, the CD47-SIRPα blockade agent comprises an immunoglobulin G (IgG) Fc domain. In some embodiments, the IgG Fc domain comprises an IgG1 Fc domain. In some embodiments, the IgG1 Fc domain comprises a fragment of a human antibody. In some embodiments, the CD47-SIRPα blockade agent is selected from the group consisting of TTI-621, TTI-622, and ALX148. In some embodiments, the CD47-SIRPα blockade agent is TTI-621, TTI-622, and ALX148. In some embodiments, the CD47-SIRPα blockade agent is T1I-622. In some embodiments, the CD47-SIRPα blockade agent is ALX148. In some embodiments, the IgG Fc domain comprises an IgG4 Fc domain. In some embodiments, the CD47-SIRPα blockade agent is an antibody. In some embodiments, the antibody is selected from the group consisting of MIAP410, B6H12, and Magrolimab. In some embodiments, the antibody is MIAP410. In some embodiments, the antibody is B6H12. In some embodiments, the antibody is Magrolimab. In some embodiments, the antibody is selected from the group consisting of AO-176, IBI188 (letaplimab), STI-6643, and ZL-1201. In some embodiments, the antibody is AO-176 (Arch). In some embodiments, the antibody is IBI188 (letaplimab) (Innovent). In some embodiments, the antibody is STI-6643 (Sorrento). In some embodiments, the antibody is ZL-1201 (Zai).

In some embodiments, useful antibodies or fragments thereof that bind CD47 can be selected from a group that includes magrolimab ((Hu5F9-G4)) (Forty Seven, Inc.; Gilead Sciences. Inc.), urabrelimab, CC-90002 (Celgene; Bristol-Myers Squibb), IBI-188 (Innovent Biologics), IBI-322 (Innovent Biologics), TG-1801 (TG Therapeutics: also known as NI-1701, Novimmune SA), ALX148 (ALX Oncology), TJ011133 (also known as TJC4, I-Mab Biopharma), FA3M3, ZL-1201 (Zai Lab Co., Ltd), AK117 (Akesbio Australia Pty, Ltd.), AO-176 (Arch Oncology), SRF231 (Surface Oncology), GenSci-059 (GeneScience), C47B157 (Janssen Research and Development), C47B161 (Janssen Research and Development), C47B167 (Janssen Research and Development), C47B222 (Janssen Research and Development), C47B227 (Janssen Research and Development), Vx-1004 (Corvus Pharmaceuticals), HMBDO04 (Hummingbird Bioscience Pte Ltd), SHR-1603 (Hengrui), AMMS4-G4 (Beijing Institute of Biotechnology). RTX-CD47 (University of Groningen), and IMC-002. (Samsung Biologics: ImmuneOncia Therapeutics). In some embodiments, the antibody or fragment thereof does not compete for CD47 binding with an antibody selected from a group that includes magrolimab, urabrelimab, CC-90002, IBI-188, IBI-322, TG-1801 (NI-1701), ALX148, TJ011133, FA3M3, ZL1201·AK117, AO-176, SRF231, GenSci-059, C47B157, C47B161, C47B167, C47B222, C47B227, Vx-1004, HMBD004, SHR-1603, AMMS4-G4, RTX-CD47, and IMC-002. In some embodiments, the antibody or fragment thereof competes for CD47 binding with an antibody selected from magrolimab, urabrelimab, CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002. In some embodiments, the antibody or fragment thereof that binds CD47 is selected from a group that includes a single-chain Fv fragment (scFv) against CD47, a Fab against CD47, a VHH nanobody against CD47, a DARPin against CD47, and variants thereof. In some embodiments, the scFv against CD47, a Fab against CD47, and variants thereof are based on the antigen binding domains of any of the antibodies selected from a group that includes magrolimab, urabrelimab, CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002.

In some embodiments, the CD47 antagonist provides CD47 blockade. Methods and agents for CD47 blockade are described in PCT/US2021/054326, which is herein incorporated by reference in its entirety.

Once altered, the presence of expression of any of the molecule described herein can be assayed using known techniques, such as Western blots, ELISA assays, FACS assays, and the like.

In some embodiments, characteristics associated with a particular cell type, for example, cell marker characterization, biomarker, intracellular markers, extracellular markers, cell cytokine production, antibody production may also be used as a readout for cell quality. In some embodiments, intracellular markers can be a change in intracellular protein level. In some embodiments, intracellular markers can be a change in intracellular RNA level. In some embodiments, intracellular markers can be a change in intracellular DNA level. In some embodiments, extracellular markers can be a change in extracellular peptide levels (e.g., one or more cytokines, one or more hormones, one or more antibodies, and the like). In some embodiments, extracellular markers can be a change in extracellular signaling molecule levels (e.g., one or more signaling peptides, one or more metabolites, one or more ligands, one or more organic compounds, one or more ions, and the like).

### A. Reduced Expression of Target Genes

### 1. Target Genes

### A. MHC Class I Molecules And/or MHC Class II Molecules

In some embodiments, the provided engineered cells comprises a modification (e.g., genetic modifications) of one or more target polynucleotide or protein sequences (also interchangeably referred to as a target gene) that regulate (e.g., reduce or eliminate) the expression of either one or more MHC class I molecules, one or more MHC class II molecules, or one or more MHC class I molecules and one or more MHC class II molecules. In some embodiments, the cell to be modified or engineered is an unmodified cell or non-engineered cell that has not previously been introduced with the one or more modifications. In some embodiments, a genetic editing system is used to modify one or more target polynucleotide sequences that regulate (e.g., reduce or eliminate) the expression of either one or more MHC class I molecules, one or more MHC class II molecules, or one or more MHC class I molecules and MHC class II molecules. In certain embodiments, the genome of the cell has been altered to reduce or delete components required or involved in facilitating HLA expression, such as expression of one or more MHC class I molecules and/or one or more MHC class II molecules on the surface of the cell. For instance, in some embodiments, expression of a beta-2-microgloublin (B2M), a component of MHC class I molecules, is reduced or eliminated in the cell, thereby reducing or elimination the protein expression (e.g., cell surface expression) of one or more MHC class I molecules by the engineered cell.

In some embodiments, any of the described modifications in the engineered cell that regulate (e.g., reduce or eliminate) expression of one or more target polynucleotide or protein in the engineered cell may be combined together with one or more modifications to overexpress a polynucleotide (e.g., tolerogenic factor, such as CD47) described in Section II.B.

In some embodiments, reduction of one or more MHC class I molecules and/or one or more MHC class II molecules expression can be accomplished, for example, by one or more of the following: (1) targeting the polymorphic HLA alleles (HLA-A, HLA-B, HLA-C) and MHC class II genes directly: (2) removal of B2M, which will reduce surface trafficking of all MHC class I molecules; and/or (3) deletion of one or more components of the MHC enhanceosomes, such as LRC5, RFX-5, RFXANK, RFXAP, IRF1, NF-Y (including NFY-A, NFY-B, NFY-C), and CIITA that are critical for HLA expression.

In certain embodiments, HLA expression is interfered with. In some embodiments, HLA expression is interfered with by targeting individual HLAs (e.g., knocking out expression of HLA-A, HLA-B and/or HLA-C), targeting transcriptional regulators of HLA expression (e.g., knocking out expression of NLRC5, CIITA, RFX5, RFXAP, RFXANK, NFY-A, NFY-B, NFY-C and/or IRF-1), blocking surface trafficking of MHC class I molecules (e.g., knocking out expression of B2M and/or TAP1), and/or targeting with HLA-Razor (see, e.g., WO2016183041).

The human leukocyte antigen (HLA) complex is synonymous with human MHC. In some embodiments, the engineered cells disclosed herein are human cells. In certain aspects, the engineered cells disclosed herein do not express one or more human leukocyte antigens (e.g., HLA-A, HLA-B and/or HLA-C) corresponding to one or more MHC class I molecules and/or one or more MHC class II molecules and are thus characterized as being hypoimmunogenic. For example, in certain aspects, the engineered cells disclosed herein have been modified such that the cells, including any stem cell or a differentiated stem cell prepared therefrom, do not express, or exhibit reduced expression of one or more of the following MHC class I molecules: HLA-A, HLA-B and HLA-C. In some embodiments, one or more of HLA-A, HLA-B and HLA-C may be “knocked-out” of a cell. A cell that has a knocked-out HLA-A gene, HLA-B gene, and/or HLA-C gene may exhibit reduced or eliminated expression of each knocked-out gene.

In certain embodiments, the expression of one or more MHC class I molecules and/or one or more MHC class II molecules is modulated by targeting and deleting a contiguous stretch of genomic DNA, thereby reducing, or eliminating expression of a target gene selected from the group consisting of B2M, CIITA, and NLRC5.

In some embodiments, the provided engineered cells comprise a modification of one or more target polynucleotide sequence that regulate one or more MHC class I molecules. Exemplary methods for reducing expression of one or more MHC class I molecules are described in sections below. In some embodiments, the targeted polynucleotide sequence is one or both of B2M and NLRC5. In some embodiments, the cell comprises a genetic editing modification (e.g., an indel) to the B2M gene. In some embodiments, the cell comprises a genetic editing modification (e.g., an indel) to the NLRC5 gene. In some embodiments, the cell comprises genetic editing modifications (e.g., indels) to the B2M and CIITA genes.

In some embodiments, a modification that reduces expression of one or more MHC class I molecules is a modification that reduces expression of B2M. In some embodiments, the modification that reduces B2M expression reduces B2M mRNA expression. In some embodiments, the reduced mRNA expression of B2M is relative to an unmodified or wild-type cell of the same cell type that does not comprise the modification. In some embodiments, the mRNA expression of B2M is reduced by more than about 5%, such as reduced by more than about any of 10%, 20%, 30%, 40%, 50%, 60%, 70%, 80%, 90%, or more. In some embodiments, the mRNA expression of B2M is reduced by up to about 100%, such as reduced by up to about any of 90%, 80%, 70%, 60%, 50%, 40%, 30%, 20%, 10%, 5%, or less. In some embodiments, the mRNA expression of B2M is reduced by any of about 5%, 10%, 20%, 30%, 40%, 50%, 60%, 70%, 80%, 90%, or 100%. In some embodiments, the mRNA expression of B2M is eliminated (e.g., 0% expression of B2M mRNA). In some embodiments, the modification that reduces B2M mRNA expression eliminates B2M gene activity.

In some embodiments, the modification that reduces B2M expression reduces B2M protein expression. In some embodiments, the reduced protein expression of B2M is relative to an unmodified or wild-type cell of the same cell type that does not comprise the modification. In some embodiments, the protein expression of B2M is reduced by more than about 5%, such as reduced by more than about any of 10%, 20%, 30%, 40%, 50%, 60%, 70%, 80%, 90%, or more. In some embodiments, the protein expression of B2M is reduced by up to about 100%, such as reduced by up to about any of 90%, 80%, 70%, 60%, 50%, 40%, 30%, 20%, 10%, 5%, or less. In some embodiments, the protein expression of B2M is reduced by any of about 5%, 10%, 20%, 30%, 40%, 50%, 60%, 70%, 80%, 90%, or 100%. In some embodiments, the protein expression of B2M is eliminated (e.g., 0% expression of B2M protein). In some embodiments, the modification that reduces B2M protein expression eliminates B2M gene activity.

In some embodiments, the modification that reduces B2M expression comprises inactivation or disruption of the B2M gene. In some embodiments, the modification that reduces B2M expression comprises inactivation or disruption of one allele of the B2M gene. In some embodiments, the modification that reduces B2M expression comprises inactivation or disruption comprises inactivation or disruption of both alleles of the B2M gene.

In some embodiments, the modification comprises inactivation or disruption of one or more B2M coding sequences in the cell. In some embodiments, the modification comprises inactivation or disruption of all B2M coding sequences in the cell. In some embodiments, the modification comprises inactivation or disruption comprises an indel in the B2M gene. In some embodiments, the modification is a frameshift mutation of genomic DNA of the B2M gene. In some embodiments, the modification is a deletion of genomic DNA of the B2M gene. In some embodiments, the modification is a deletion of a contiguous stretch of genomic DNA of the B2M gene. In some embodiments, the B2M gene is knocked out.

In some embodiments, the provided engineered cells comprise a modification of one or more target polynucleotide sequence that regulate one or more MHC class II molecules. Exemplary methods for reducing expression of one or more MHC class II molecules are described in sections below. In some embodiments, the cell comprises a genetic editing modification to the CIITA gene.

In some embodiments, a modification that reduces expression of one or more MHC class II molecules is a modification that reduces expression of CIITA. In some embodiments, the modification that reduces CIITA expression reduces CIITA mRNA expression. In some embodiments, the reduced mRNA expression of CIITA is relative to an unmodified or wild-type cell of the same cell type that does not comprise the modification. In some embodiments, the mRNA expression of CIITA is reduced by more than about 5%, such as reduced by more than about any of 10%, 20%, 30%, 40%, 50%, 60%, 70%, 80%, 90%, or more. In some embodiments, the mRNA expression of CIITA is reduced by up to about 100%, such as reduced by up to about any of 90%, 80%, 70%, 60%, 50%, 40%, 30%, 20%, 10%, 5%, or less. In some embodiments, the mRNA expression of CIITA is reduced by any of about 5%, 10%, 20%, 30%, 40%, 50%, 60%, 70%, 80%, 90%, or 100%. In some embodiments, the mRNA expression of CIITA is eliminated (e.g., 0% expression of CIITA mRNA). In some embodiments, the modification that reduces CIITA mRNA expression eliminates CIITA gene activity.

In some embodiments, the modification that reduces CIITA expression reduces CIITA protein expression. In some embodiments, the reduced protein expression of CIITA is relative to an unmodified or wild-type cell of the same cell type that does not comprise the modification. In some embodiments, the protein expression of CIITA is reduced by more than about 5%, such as reduced by more than about any of 10%, 20%, 30%, 40%, 50%, 60%, 70%, 80%, 90%, or more. In some embodiments, the protein expression of CIITA is reduced by up to about 100%, such as reduced by up to about any of 90%, 80%, 70%, 60%, 50%, 40%, 30%, 20%, 10%, 5%, or less. In some embodiments, the protein expression of CIITA is reduced by any of about 5%, 10%, 20%, 30%, 40%, 50%, 60%, 70%, 80%, 90%, or 100%. In some embodiments, the protein expression of CIITA is eliminated (e.g., 0% expression of CIITA protein). In some embodiments, the modification that reduces CIITA protein expression eliminates CIITA gene activity.

In some embodiments, the modification that reduces CIITA expression comprises inactivation or disruption of the CIITA gene. In some embodiments, the modification that reduces CIITA expression comprises inactivation or disruption of one allele of the CIITA gene. In some embodiments, the modification that reduces CIITA expression comprises inactivation or disruption comprises inactivation or disruption of both alleles of the CIITA gene.

In some embodiments, the modification comprises inactivation or disruption of one or more B2M coding sequences in the cell. In some embodiments, the modification comprises inactivation or disruption of all B2M coding sequences in the cell. In some embodiments, the modification comprises inactivation or disruption comprises an indel in the B2M gene. In some embodiments, the modification is a frameshift mutation of genomic DNA of the B2M gene. In some embodiments, the modification is a deletion of genomic DNA of the B2M gene. In some embodiments, the modification is a deletion of a contiguous stretch of genomic DNA of the B2M gene. In some embodiments, the CIITA gene is knocked out.

In some embodiments, the provided engineered cells comprise a modification of one or more target polynucleotide sequence that regulate one or more MHC class I molecules and/or one or more MHC class II molecules. Exemplary methods for reducing expression of one or more MHC class I molecules and/or one or more MHC class II molecules are described in sections below. In some embodiments, the cell comprises genetic editing modifications to the B2M and NLRC5 genes. In some embodiments, the cell comprises genetic editing modifications to the CIITA and NLRC5 genes. In embodiments, the cell comprises genetic editing modifications to the B2M, CIITA and NLRC5 genes.

### 2. Methods of Reducing Expression

In some embodiments, the cells provided herein are modified (e.g., genetically modified) to reduce expression of the one or more target polynucleotides or proteins as described. In some embodiments, the cell that is engineered with the one or more modification to reduce (e.g., eliminate) expression of a polynucleotide or protein is any source cell as described herein. In some embodiments, the source cell is any cell described in Section II.C. In certain embodiments, the cells (e.g., stem cells, induced pluripotent stem cells, differentiated cells such as beta islet cells or hepatocytes, or primary cells) disclosed herein comprise one or more modifications to reduce expression of one or more target polynucleotides. Non-limiting examples of the one or more target polynucleotides include any as described above, such as one or more of CIITA, B2M, NLRC5, HLA-A, HLA-B, HLA-C, LRC5, RFX-ANK, RFX5, RFX-AP, NFY-A, NFY-B, NFY-C, IRF1, and TAP1. In some embodiments, the modifications to reduce expression of the one or more target polynucleotides are combined with one or more modifications to increase expression of a desired transgene, such as any described in Section II.B. In some embodiments, the modifications create engineered cells that are immune-privileged or hypoimmunogenic cells. By modulating (e.g., reducing or deleting) expression of one or a plurality of the target polynucleotides, such cells exhibit decreased immune activation when engrafted into a recipient subject. In some embodiments, the cell is considered hypoimmunogenic, e.g., in a recipient subject or patient upon administration.

Any method for reducing expression of a target polynucleotide may be used. In some embodiments, the modifications result in permanent elimination or reduction in expression of the target polynucleotide. For instance, in some embodiments, the target polynucleotide or gene is disrupted by introducing a DNA break in the target polynucleotide, such as by using a targeting endonuclease. In other embodiments, the modifications result in transient reduction in expression of the target polynucleotide. For instance, in some embodiments gene repression is achieved using an inhibitory nucleic acid that is complementary to the target polynucleotide to selectively suppress or repress expression of the gene, for instance using antisense techniques, such as by RNA interference (RNAi), short interfering RNA (siRNA), short hairpin (shRNA), and/or ribozymes.

In some embodiments, the target polynucleotide sequence is a genomic sequence. In some embodiments, the target polynucleotide sequence is a human genomic sequence. In some embodiments, the target polynucleotide sequence is a mammalian genomic sequence. In some embodiments, the target polynucleotide sequence is a vertebrate genomic sequence.

In some embodiments, gene disruption is carried out by induction of one or more double-stranded breaks and/or one or more single-stranded breaks in the gene, typically in a targeted manner. In some embodiments, the double-stranded or single-stranded breaks are made by a nuclease, e.g., an endonuclease, such as a gene-targeted nuclease. In some embodiments, the targeted nuclease is selected from zinc finger nucleases (ZFN), transcription activator-like effector nucleases (TALENs), and RNA-guided nucleases such as a CRISPR-associated nuclease (Cas), specifically designed to be targeted to the sequence of a gene or a portion thereof. In some embodiments, the targeted nuclease generates double-stranded or single-stranded breaks that then undergo repair through error prone non-homologous end joining (NHEJ) or, in some cases, precise homology directed repair (HDR) in which a template is used. In some embodiments, the targeted nuclease generates DNA double strand breaks (DSBs). In some embodiments, the process of producing and repairing the breaks is typically error prone and results in insertions and deletions (indels) of DNA bases from NHEJ repair. In some embodiments, the modification may induce a deletion, insertion, or mutation of the nucleotide sequence of the target gene. In some cases, the modification may result in a frameshift mutation, which can result in a premature stop codon. In examples of nuclease-mediated gene editing the targeted edits occur on both alleles of the gene resulting in a biallelic disruption or edit of the gene. In some embodiments, all alleles of the gene are targeted by the gene editing. In some embodiments, modification with a targeted nuclease, such as using a CRISPR/Cas system, leads to complete knockout of the gene.

In some embodiments, the nuclease, such as a rare-cutting endonuclease, is introduced into a cell containing the target polynucleotide sequence. The nuclease may be introduced into the cell in the form of a nucleic acid encoding the nuclease. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid that is introduced into the cell is DNA. In some embodiments, the nuclease is introduced into the cell in the form of a protein. For instance, in the case of a CRISPR/Cas system a ribonucleoprotein (RNP) may be introduced into the cell.

In some embodiments, the modification occurs using a CRISPR/Cas system. Any CRISPR/Cas system that is capable of altering a target polynucleotide sequence in a cell can be used. Such CRISPR-Cas systems can employ a variety of Cas proteins (Haft et al. PLoS Comput Biol. 2005; 1(6)e60). The molecular machinery of such Cas proteins that allows the CRISPR/Cas system to alter target polynucleotide sequences in cells include RNA binding proteins, endo- and exo-nucleases, helicases, and polymerases. In some embodiments, the CRISPR/Cas system is a CRISPR type I system. In some embodiments, the CRISPR/Cas system is a CRISPR type II system. In some embodiments, the CRISPR/Cas system is a CRISPR type V system.

The CRISPR/Cas systems include targeted systems that can be used to alter any target polynucleotide sequence in a cell. In some embodiments, a CRISPR/Cas system provided herein includes a Cas protein and one or more, such as at least one to two, ribonucleic acids (e.g., guide RNA (gRNA)) that are capable of directing the Cas protein to and hybridizing to a target motif of a target polynucleotide sequence.

In some embodiments, a Cas protein comprises one or more amino acid substitutions or modifications. In some embodiments, the one or more amino acid substitutions comprises a conservative amino acid substitution. In some instances, substitutions and/or modifications can prevent or reduce proteolytic degradation and/or extend the half-life of the polypeptide in a cell. In some embodiments, the Cas protein can comprise a peptide bond replacement (e.g., urea, thiourea, carbamate, sulfonyl urea, etc.). In some embodiments, the Cas protein can comprise a naturally occurring amino acid. In some embodiments, the Cas protein can comprise an alternative amino acid (e.g., D-amino acids, beta-amino acids, homocysteine, phosphoserine, etc.). In some embodiments, a Cas protein can comprise a modification to include a moiety (e.g., PEGylation, glycosylation, lipidation, acetylation, end-capping, etc.).

In some embodiments, a Cas protein comprises a core Cas protein. Exemplary Cas core proteins include, but are not limited to Cas1, Cas2, Cas3, Cas4, Cas5, Cas6, Cas7, Cas8 and Cas9. In some embodiments, a Cas protein comprises a Cas protein of an E. coli subtype (also known as CASS2). Exemplary Cas proteins of the E. Coli subtype include, but are not limited to Cse1, Cse2, Cse3, Cse4, and Cas5e. In some embodiments, a Cas protein comprises a Cas protein of the Ypest subtype (also known as CASS3). Exemplary Cas proteins of the Ypest subtype include, but are not limited to Csy1, Csy2, Csy3, and Csy4. In some embodiments, a Cas protein comprises a Cas protein of the Nmeni subtype (also known as CASS4). Exemplary Cas proteins of the Nmeni subtype include but are not limited to Csn1 and Csn2. In some embodiments, a Cas protein comprises a Cas protein of the Dvulg subtype (also known as CASS1). Exemplary Cas proteins of the Dvulg subtype include Csd1, Csd2, and Cas5d. In some embodiments, a Cas protein comprises a Cas protein of the Tneap subtype (also known as CASS7). Exemplary Cas proteins of the Tneap subtype include, but are not limited to, Cst1, Cst2, Cas5t. In some embodiments, a Cas protein comprises a Cas protein of the Hmari subtype. Exemplary Cas proteins of the Hmari subtype include, but are not limited to Csh1, Csh2, and Cas5h. In some embodiments, a Cas protein comprises a Cas protein of the Apem subtype (also known as CASS5). Exemplary Cas proteins of the Apem subtype include, but are not limited to Csa1, Csa2, Csa3, Csa4, Csa5, and Cas5a. In some embodiments, a Cas protein comprises a Cas protein of the Mtube subtype (also known as CASS6). Exemplary Cas proteins of the Mtube subtype include, but are not limited to Csm1, Csm2, Csm3, Csm4, and Csm5. In some embodiments, a Cas protein comprises a RAMP module Cas protein. Exemplary RAMP module Cas proteins include, but are not limited to, Cmr1, Cmr2, Cmr3, Cmr4, Cmr5, and Cmr6. See, e.g., Klompe et al., Nature 571. 219-225 (2019); Strecker et al., Science 365, 48-53 (2019).

In some embodiments, CRISPR systems of the present disclosure comprise TnpB polypeptides. In some embodiments, TnpB polypeptides may comprise a Ruv-C-like domain. The RuvC domain may be a split RuvC domain comprising RuvC-I, RuvC-II, and RuvC-III subdomains. In some embodiments, a TnpB may further comprise one or more of a HTH domain, a bridge helix domain, and a zinc finger domain. TnpB polypeptides do not comprise an HNH domain. In some embodiments, a TnpB protein comprises, starting at the N-terminus: a HTH domain, a RuvC-I subdomain, a bridge helix domain, a RuvC-II sub-domain, a zinger finger domain, and a RuvC-III sub-domain. In some embodiments, a RuvC-III sub-domain forms the C-terminus of a TnpB polypeptide. In some embodiments, a TnpB polypeptide is from Epsilonproteobacteria bacterium, Actinoplanes lobatus strain DSM 43150, Actinomadura celluolosilytica strain DSM 45823, Actinomadura namibiensis strain DSM 44197, Alicyclobacillus macrosprangiidus strain DSM 17980, Lipingzhangella halophila strain DSM 102030, or Ktedonobacter racemifer. In some embodiments, a TnpB polypeptide is from Ktedonobacter racemifer, or comprises a conserved RNA region with similarity to the 5′ ITR of K. racemifer TnpB loci. In some embodiments, a TnpB may comprise a Fanzor protein, a TnpB homolog found in eukaryotic genomes. In some embodiments, a CRISPR system comprising a TnpB polypeptide binds a target adjacent motif (TAM) sequence 5′ of a target polynucleotide. In some embodiments, a TAM is a transposon-associated motif. In some embodiments, a TAM sequence comprises TCA. In some embodiments, a TAM sequence comprises TTCAN. In some embodiments, a TAM sequence comprises TTGAT. In some embodiments, a TAM sequence comprises ATAAA.

In some embodiments, the methods for genetically modifying cells to knock out, knock down, or otherwise modify one or more genes comprise using a site-directed nuclease, including, for example, zinc finger nucleases (ZFNs), transcription activator-like effector nucleases (TALENs), meganucleases, transposases, and clustered regularly interspaced short palindromic repeat (CRISPR)/Cas systems

ZFNs are fusion proteins comprising an array of site-specific DNA binding domains adapted from zinc finger-containing transcription factors attached to the endonuclease domain of the bacterial FokI restriction enzyme. A ZFN may have one or more (e.g., 1, 2, 3, 4, 5, 6, 7, 8, 9, 10, or more) of the DNA binding domains or zinc finger domains. See, e.g., Carroll et al., Genetics Society of America (2011) 188:773-782; Kim et al., Proc. Natl. Acad. Sci. USA (1996) 93:1156-1160. Each zinc finger domain is a small protein structural motif stabilized by one or more zinc ions and usually recognizes a 3- to 4-bp DNA sequence. Tandem domains can thus potentially bind to an extended nucleotide sequence that is unique within a cell's genome.

Various zinc fingers of known specificity can be combined to produce multi-finger polypeptides which recognize about 6, 9, 12, 15, or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Zinc fingers can be engineered to bind a predetermined nucleic acid sequence. Criteria to engineer a zinc finger to bind to a predetermined nucleic acid sequence are known in the art. See, e.g., Sera et al., Biochemistry (2002) 41:7074-7081; Liu et al., Bioinformatics (2008) 24:1850-1857.

ZFNs containing FokI nuclease domains or other dimeric nuclease domains function as a dimer. Thus, a pair of ZFNs are required to target non-palindromic DNA sites. The two individual ZFNs must bind opposite strands of the DNA with their nucleases properly spaced apart. See Bitinaite et al., Proc. Natl. Acad. Sci. USA (1998) 95:10570-10575. To cleave a specific site in the genome, a pair of ZFNs are designed to recognize two sequences flanking the site, one on the forward strand and the other on the reverse strand. Upon binding of the ZFNs on either side of the site, the nuclease domains dimerize and cleave the DNA at the site, generating a DSB with 5′ overhangs. HDR can then be utilized to introduce a specific mutation, with the help of a repair template containing the desired mutation flanked by homology arms. The repair template is usually an exogenous double-stranded DNA vector introduced to the cell. See Miller et al., Nat. Biotechnol. (2011) 29:143-148; Hockemeyer et al., Nat. Biotechnol. (2011) 29:731-734.

TALENs are another example of an artificial nuclease which can be used to edit a target gene. TALENs are derived from DNA binding domains termed TALE repeats, which usually comprise tandem arrays with 10 to 30 repeats that bind and recognize extended DNA sequences. Each repeat is 33 to 35 amino acids in length, with two adjacent amino acids (termed the repeat-variable di-residue, or RVD) conferring specificity for one of the four DNA base pairs. Thus, there is a one-to-one correspondence between the repeats and the base pairs in the target DNA sequences.

TALENs are produced artificially by fusing one or more TALE DNA binding domains (e.g., 1, 2, 3, 4, 5, 6, 7, 8, 9, 10 or more) to a nuclease domain, for example, a FokI endonuclease domain. See Zhang, Nature Biotech. (2011) 29:149-153. Several mutations to FokI have been made for its use in TALENs; these, for example, improve cleavage specificity or activity. See Cermak et al., Nucl. Acids Res. (2011) 39:e82: Miller et al., Nature Biotech. (2011) 29:143-148; Hockemeyer et al., Nature Biotech. (2011) 29:731-734; Wood et al., Science (2011) 333:307; Doyon et al., Nature Methods (2010) 8:74-79; Szczepek et al., Nature Biotech (2007)25:786-793; Guo et al., J Mol. Biol. (2010)200:96. The FokI domain functions as a dimer, requiring two constructs with unique DNA binding domains for sites in the target genome with proper orientation and spacing. Both the number of amino acid residues between the TALE DNA binding domain and the FokI nuclease domain and the number of bases between the two individual TALEN binding sites appear to be important parameters for achieving high levels of activity. Miller et al., Nature Biotech. (2011) 29:143-148.

By combining engineered TALE repeats with a nuclease domain, a site-specific nuclease can be produced specific to any desired DNA sequence. Similar to ZFNs, TALENs can be introduced into a cell to generate DSBs at a desired target site in the genome, and so can be used to knock out genes or knock in mutations in similar, HDR-mediated pathways. See Boch, Nature Biotech. (2011) 29:135-136; Boch et al., Science (2009) 326:1509-1512; Moscou et al., Science (2009) 326:3501.

Meganucleases are enzymes in the endonuclease family which are characterized by their capacity to recognize and cut large DNA sequences (from 14 to 40 base pairs). Meganucleases are grouped into families based on their structural motifs which affect nuclease activity and/or DNA recognition. The most widespread and best known meganucleases are the proteins in the LAGLIDADG family, which owe their name to a conserved amino acid sequence. See Chevalier et al., Nucleic Acids Res. (2001) 29(18); 3757-3774. On the other hand, the GIY-YIG family members have a GIY-YIG module, which is 70-100 residues long and includes four or five conserved sequence motifs with four invariant residues, two of which are required for activity. See Van Roey et al., Nature Struct. Biol. (2002) 9:806-811. The His-Cys family meganucleases are characterized by a highly conserved series of histidines and cysteines over a region encompassing several hundred amino acid residues. See Chevalier et al., Nucleic Acids Res. (2001) 29(18):3757-3774. Members of the NHN family are defined by motifs containing two pairs of conserved histidines surrounded by asparagine residues. See Chevalier et al., Nucleic Acids Res. (2001) 29(18):3757-3774.

Because the chance of identifying a natural meganuclease for a particular target DNA sequence is low due to the high specificity requirement, various methods including mutagenesis and high throughput screening methods have been used to create meganuclease variants that recognize unique sequences. Strategies for engineering a meganuclease with altered DNA-binding specificity, e.g., to bind to a predetermined nucleic acid sequence are known in the art. See, e.g., Chevalier et al., Mol. Cell. (2002) 10:895-905; Epinat et al., Nucleic Acids Res (2003) 31:2952-2962; Silva et al., J Mol. Biol. (2006) 361:744-754; Seligman et al., Nucleic Acids Res (2002) 30:3870-3879; Sussman et al., J Mol Biol (2004) 342:31-41; Doyon et al., J Am Chem Soc (2006) 128:2477-2484; Chen et al., Protein Eng Des Sel (2009) 22:249-256; Amould et al., J Mol Biol. (2006) 355:443-458: Smith et al., Nucleic Acids Res. (2006) 363(2):283-294.

Like ZFNs and TALENs, Meganucleases can create DSBs in the genomic DNA, which can create a frame-shift mutation if improperly repaired, e.g., via NHEJ, leading to a decrease in the expression of a target gene in a cell. Alternatively, foreign DNA can be introduced into the cell along with the meganuclease. Depending on the sequences of the foreign DNA and chromosomal sequence, this process can be used to modify the target gene. See Silva et al., Current Gene Therapy (2011) 11:11-27.

Transposases are enzymes that bind to the end of a transposon and catalyze its movement to another part of the genome by a cut and paste mechanism or a replicative transposition mechanism. By linking transposases to other systems such as the CRISPER/Cas system, new gene editing tools can be developed to enable site specific insertions or manipulations of the genomic DNA. There are two known DNA integration methods using transposons which use a catalytically inactive Cas effector protein and Tn7-like transposons. The transposase-dependent DNA integration does not provoke DSBs in the genome, which may guarantee safer and more specific DNA integration.

The CRISPR system was originally discovered in prokaryotic organisms (e.g., bacteria and archaea) as a system involved in defense against invading phages and plasmids that provides a form of acquired immunity. Now it has been adapted and used as a popular gene editing tool in research and clinical applications.

CRISPR/Cas systems generally comprise at least two components: one or more guide RNAs (gRNAs) and a Cas protein. The Cas protein is a nuclease that introduces a DSB into the target site. CRISPR-Cas systems fall into two major classes: class 1 systems use a complex of multiple Cas proteins to degrade nucleic acids; class 2 systems use a single large Cas protein for the same purpose. Class 1 is divided into types I, III, and IV: class 2 is divided into types II, V, and VI. Different Cas proteins adapted for gene editing applications include, but are not limited to, Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. The most widely used Cas9 is a type II Cas protein and is described herein as illustrative. These Cas proteins may be originated from different source species. For example, Cas9 can be derived from S. pyogenes or S. aureus.

In the original microbial genome, the type II CRISPR system incorporates sequences from invading DNA between CRISPR repeat sequences encoded as arrays within the host genome. Transcripts from the CRISPR repeat arrays are processed into CRISPR RNAs (crRNAs) each harboring a variable sequence transcribed from the invading DNA, known as the “protospacer” sequence, as well as part of the CRISPR repeat. Each crRNA hybridizes with a second transactivating CRISPR RNA (tracrRNA), and these two RNAs form a complex with the Cas9 nuclease. The protospacer-encoded portion of the crRNA directs the Cas9 complex to cleave complementary target DNA sequences, provided that they are adjacent to short sequences known as “protospacer adjacent motifs” (PAMs).

Since its discovery, the CRISPR system has been adapted for inducing sequence specific DSBs and targeted genome editing in a wide range of cells and organisms spanning from bacteria to eukaryotic cells including human cells. In its use in gene editing applications, artificially designed, synthetic gRNAs have replaced the original crRNA:tracrRNA complex. For example, the gRNAs can be single guide RNAs (sgRNAs) composed of a crRNA, a tetraloop, and a tracrRNA. The crRNA usually comprises a complementary region (also called a spacer, usually about 20 nucleotides in length) that is user-designed to recognize a target DNA of interest. The tracrRNA sequence comprises a scaffold region for Cas nuclease binding. The crRNA sequence and the tracrRNA sequence are linked by the tetraloop and each have a short repeat sequence for hybridization with each other, thus generating a chimeric sgRNA. One can change the genomic target of the Cas nuclease by simply changing the spacer or complementary region sequence present in the gRNA. The complementary region will direct the Cas nuclease to the target DNA site through standard RNA-DNA complementary base pairing rules.

In order for the Cas nuclease to function, there must be a PAM immediately downstream of the target sequence in the genomic DNA. Recognition of the PAM by the Cas protein is thought to destabilize the adjacent genomic sequence, allowing interrogation of the sequence by the gRNA, and resulting in gRNA-DNA pairing when a matching sequence is present. The specific sequence of PAM varies depending on the species of the Cas gene. For example, the most commonly used Cas9 nuclease derived from S. pyogenes recognizes a PAM sequence of 5′-NGG-3′ or, at less efficient rates, 5′-NAG-3′, where “N” can be any nucleotide. Other Cas nuclease variants with alternative PAMs have also been characterized and successfully used for genome editing, which are summarized in Table 1a below.

TABLE la
Exemplary Cas nuclease variants and their PAM sequences

| CRISPR Nuclease | Source Organism | PAM Sequence (5′→3′) |
|---|---|---|
| SpCas9 | Streptococcus pyogenes | NGG or NAG |
| SaCas9 | Staphylococcus aureus | NGRRT or NGRRN |
| NmeCas9 | Neisseria meningitidis | NNNNGATT |
| CjCas9 | Campylobacter jejuni | NNNNRYAC |
| StCas9 | Streptococcus thermophilus | NNAGAAW |
| TdCas9 | Treponema denticola | NAAAAC |
| LbCas12a (Cpf1) | Lachnospiraceae bacterium | TTTV |
| AsCas12a (Cpf1) | Acidaminococcus sp. | TTTV |
| AacCas12b | Alicyclobacillus acidiphilus | TTN |
| BhCas12b v4 | Bacillus hisashii | ATTN, TTTN, or GTTN |

R = A or G; Y = C or T; W = A or T; V = A or C or G; N = any base

In some embodiments, Cas nucleases may comprise one or more mutations to alter their activity, specificity, recognition, and/or other characteristics. For example, the Cas nuclease may have one or more mutations that alter its fidelity to mitigate off-target effects (e.g., eSpCas9, SpCas9-HF1, HypaSpCas9. HeFSpCas9, and evoSpCas9 high-fidelity variants of SpCas9). For another example, the Cas nuclease may have one or more mutations that alter its PAM specificity.

In some embodiments, a Cas protein comprises any one of the Cas proteins described herein or a functional portion thereof. As used herein, “functional portion” refers to a portion of a peptide which retains its ability to complex with at least one ribonucleic acid (e.g., guide RNA (gRNA)) and cleave a target polynucleotide sequence. In some embodiments, the functional portion comprises a combination of operably linked Cas9 protein functional domains selected from the group consisting of a DNA binding domain, at least one RNA binding domain, a helicase domain, and an endonuclease domain. In some embodiments, the functional portion comprises a combination of operably linked Cas12a (also known as Cpf1) protein functional domains selected from the group consisting of a DNA binding domain, at least one RNA binding domain, a helicase domain, and an endonuclease domain. In some embodiments, the functional domains form a complex. In some embodiments, a functional portion of the Cas9 protein comprises a functional portion of a RuvC-like domain. In some embodiments, a functional portion of the Cas9 protein comprises a functional portion of the HNH nuclease domain. In some embodiments, a functional portion of the Cas12a protein comprises a functional portion of a RuvC-like domain.

In some embodiments, suitable Cas proteins include, but are not limited to. Cas0, Cas12a (i.e., Cpf1), Cas12b, Cas12i, CasX, and Mad7.

In some embodiments, exogenous Cas protein can be introduced into the cell in polypeptide form. In certain embodiments, Cas proteins can be conjugated to or fused to a cell-penetrating polypeptide or cell-penetrating peptide. As used herein, “cell-penetrating polypeptide” and “cell-penetrating peptide” refers to a polypeptide or peptide, respectively, which facilitates the uptake of molecule into a cell. The cell-penetrating polypeptides can contain a detectable label.

In certain embodiments, Cas proteins can be conjugated to or fused to a charged protein (e.g., that carries a positive, negative, or overall neutral electric charge). Such linkage may be covalent. In some embodiments, the Cas protein can be fused to a superpositively charged GFP to significantly increase the ability of the Cas protein to penetrate a cell (Cronican et al. ACS Chem Biol. 2010; 5(8):747-52). In certain embodiments, the Cas protein can be fused to a protein transduction domain (PTD) to facilitate its entry into a cell. Exemplary PTDs include Tat, oligoarginine, and penetratin. In some embodiments, the Cas9 protein comprises a Cas9 polypeptide fused to a cell-penetrating peptide. In some embodiments, the Cas9 protein comprises a Cas9 polypeptide fused to a PTD. In some embodiments, the Cas9 protein comprises a Cas9 polypeptide fused to a tat domain. In some embodiments, the Cas9 protein comprises a Cas9 polypeptide fused to an oligoarginine domain. In some embodiments, the Cas9 protein comprises a Cas9 polypeptide fused to a penetratin domain. In some embodiments, the Cas9 protein comprises a Cas9 polypeptide fused to a superpositively charged GFP. In some embodiments, the Cas12a protein comprises a Cas12a polypeptide fused to a cell-penetrating peptide. In some embodiments, the Cas12a protein comprises a Cas12a polypeptide fused to a PTD. In some embodiments, the Cas12a protein comprises a Cas12a polypeptide fused to a tat domain. In some embodiments, the Cas12a protein comprises a Cas12a polypeptide fused to an oligoarginine domain. In some embodiments, the Cas12a protein comprises a Cas12a polypeptide fused to a penetratin domain. In some embodiments, the Cas12a protein comprises a Cas12a polypeptide fused to a superpositively charged GFP.

In some embodiments, the Cas protein can be introduced into a cell containing the target polynucleotide sequence in the form of a nucleic acid encoding the Cas protein. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as described herein. In some embodiments, the nucleic acid comprises mRNA. In some embodiments, the nucleic acid comprises a modified mRNA, as described herein (e.g., a synthetic, modified mRNA).

In provided embodiments, a CRISPR/Cas system generally includes two components: one or more guide RNA (gRNA) and a Cas protein. In some embodiments, the Cas protein is complexed with the one or more, such as one to two, ribonucleic acids (e.g., guide RNA (gRNA)). In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

In some embodiments, gRNAs are short synthetic RNAs composed of a scaffold sequence for Cas binding and a user-designed spacer or complementary portion designated crRNA. The cRNA is composed of a crRNA targeting sequence (herein after also called a gRNA targeting sequence; usually about 20 nucleotides in length) that defines the genomic target to be modified and a region of crRNA repeat (e.g., GUUUUAGAGCUA; SEQ ID NO:19). One can change the genomic target of the Cas protein by simply changing the complementary portion sequence (e.g., gRNA targeting sequence) present in the gRNA. In some embodiments the scaffold sequence for Cas binding is made up of a tracrRNA sequence (e.g., UAGCAAGUUAAAAUAAGGCUAGUCCGUUAUCAACUUGAAAAAGUGGCACCGA GUCGGUGCUUU; SEQ ID NO: 20) that hybridizes to the crRNA through its anti-repeat sequence. The complex between crRNA:tracrRNA recruits the Cas nuclease (e.g., Cas9) and cleaves upstream of a protospacer-adjacent motif (PAM). For the Cas protein to function, there must be a PAM immediately downstream of the target sequence in the genomic DNA. Recognition of the PAM by the Cas protein is thought to destabilize the adjacent genomic sequence, allowing interrogation of the sequence by the gRNA, and resulting in gRNA-DNA pairing when a matching sequence is present. The specific sequence of PAM varies depending on the species of the Cas gene. For example, the most commonly used Cas9 nuclease, derived from S. pyogenes, recognizes a PAM sequence of NGG. Other Cas9 variants and other nucleases with alternative PAMs have also been characterized and successfully used for genome editing. Thus, the CRISPR/Cas system can be used to create targeted DSBs at specified genomic loci that are complementary to the gRNA designed for the target loci. The crRNA and tracrRNA can be linked together with a loop sequence (e.g., a tetraloop; GAAA) for generation of a gRNA that is a chimeric single guide RNA (sgRNA; Hsu et al. 2013). sgRNA can be generated for DNA-based expression or by chemical synthesis.

In some embodiments, the complementary portion sequences (e.g., gRNA targeting sequence) of the gRNA will vary depending on the target site of interest. In some embodiments, the gRNAs comprise complementary portions specific to a sequence of a gene set forth in Table 1a. In some embodiments, the genomic locus targeted by the gRNAs is located within 4000 bp, within 3500 bp, within 3000 bp, within 2500 bp, within 2000 bp, within 1500 bp, within 1000 bp, or within 500 bp of any of the loci as described.

The methods disclosed herein contemplate the use of any ribonucleic acid that is capable of directing a Cas protein to and hybridizing to a target motif of a target polynucleotide sequence. In some embodiments, at least one of the ribonucleic acids comprises

In some embodiments, the Cas protein is complexed with one to two ribonucleic acids (e.g., guide RNA (gRNA)). In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

The methods disclosed herein contemplate the use of any ribonucleic acid that is capable of directing a Cas protein to and hybridizing to a target motif of a target polynucleotide sequence. In some embodiments, at least one of the ribonucleic acids comprises tracrRNA. In some embodiments, at least one of the ribonucleic acids comprises CRISPR RNA (crRNA). In some embodiments, a single ribonucleic acid comprises a guide RNA that directs the Cas protein to and hybridizes to a target motif of the target polynucleotide sequence in a cell. In some embodiments, at least one of the ribonucleic acids comprises a guide RNA that directs the Cas protein to and hybridizes to a target motif of the target polynucleotide sequence in a cell. In some embodiments, both of the one to two ribonucleic acids comprise a guide RNA that directs the Cas protein to and hybridizes to a target motif of the target polynucleotide sequence in a cell. The ribonucleic acids provided herein can be selected to hybridize to a variety of different target motifs, depending on the particular CRISPR/Cas system employed, and the sequence of the target polynucleotide, as will be appreciated by those skilled in the art. The one to two ribonucleic acids can also be selected to minimize hybridization with nucleic acid sequences other than the target polynucleotide sequence. In some embodiments, the one to two ribonucleic acids hybridize to a target motif that contains at least two mismatches when compared with all other genomic nucleotide sequences in the cell. In some embodiments, the one to two ribonucleic acids hybridize to a target motif that contains at least one mismatch when compared with all other genomic nucleotide sequences in the cell. In some embodiments, the one to two ribonucleic acids are designed to hybridize to a target motif immediately adjacent to a deoxyribonucleic acid motif recognized by the Cas protein. In some embodiments, each of the one to two ribonucleic acids are designed to hybridize to target motifs immediately adjacent to deoxyribonucleic acid motifs recognized by the Cas protein which flank a mutant allele located between the target motifs.

In some embodiments, each of the one to two ribonucleic acids comprises guide RNAs that directs the Cas protein to and hybridizes to a target motif of the target polynucleotide sequence in a cell.

In some embodiments, one or two ribonucleic acids (e.g., guide RNAs) are complementary to and/or hybridize to sequences on the same strand of a target polynucleotide sequence. In some embodiments, one or two ribonucleic acids (e.g., guide RNAs) are complementary to and/or hybridize to sequences on the opposite strands of a target polynucleotide sequence. In some embodiments, the one or two ribonucleic acids (e.g., guide RNAs) are not complementary to and/or do not hybridize to sequences on the opposite strands of a target polynucleotide sequence. In some embodiments, the one or two ribonucleic acids (e.g., guide RNAs) are complementary to and/or hybridize to overlapping target motifs of a target polynucleotide sequence. In some embodiments, the one or two ribonucleic acids (e.g., guide RNAs) are complementary to and/or hybridize to offset target motifs of a target polynucleotide sequence.

In some embodiments, nucleic acids encoding Cas protein and nucleic acids encoding the at least one to two ribonucleic acids are introduced into a cell via viral transduction (e.g., lentiviral transduction). In some embodiments, the Cas protein is complexed with 1-2 ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

Exemplary gRNA targeting sequences useful for CRISPR/Cas-based targeting of genes described herein are provided in Table 1. The sequences can be found in WO2016183041 filed May 9, 2016, the disclosure of which including the Tables, Appendices, and Sequence Listing is incorporated herein by reference in its entirety.

TABLE 1
Exemplary gRNA targeting sequences useful for targeting genes

| Gene Name | SEQ ID NO: | WO2016183041 |
|---|---|---|
| HLA-A | SEQ ID NOs: 2-1418 | Table 8, Appendix 1 |
| HLA-B | SEQ ID NOs: 1419-3277 | Table 9, Appendix 2 |
| HLA-C | SEQ ID NOS: 3278-5183 | Table 10, Appendix 3 |
| RFX-ANK | SEQ ID NOs: 95636-102318 | Table 11, Appendix 4 |
| NFY-A | SEQ ID NOs: 102319-121796 | Table 13, Appendix 6 |
| RFX5 | SEQ ID NOs: 85645-90115 | Table 16, Appendix 9 |
| RFX-AP | SEQ ID NOs: 90116-95635 | Table 17, Appendix 10 |
| NFY-B | SEQ ID NOs: 121797-135112 | Table 20, Appendix 13 |
| NFY-C | SEQ ID NOs: 135113-176601 | Table 22, Appendix 15 |
| IRF1 | SEQ ID NOs: 176602-182813 | Table 23, Appendix 16 |
| TAP1 | SEQ ID NOs: 182814-188371 | Table 24, Appendix 17 |
| CIITA | SEQ ID NOS: 5184-36352 | Table 12, Appendix 5 |
| B2M | SEQ ID NOS: 81240-85644 | Table 15, Appendix 8 |
| NLRC5 | SEQ ID NOS: 36353-81239 | Table 14, Appendix 7 |

Additional exemplary Cas9 guide RNA sequences useful for CRISPR/Cas-based targeting of genes described herein are provided in Table 2A.

TABLE 2A
Additional exemplary Cas9 guide RNA sequences useful for targeting genes

| | | | Target | | SEQ |
|---|---|---|---|---|---|
| Gene | guide sequence | PAM | site | gRNA cut location | ID NO |
| ABO | UCUCUCCAUGUGCAGUAGGA | AGG | Exon 7 | chr9: 133,257,541 | 38 |
| FUT1 | CUGGAUGUCGGAGGAGUACG | CGG | Exon 4 | chr19: 48,750,822 | 39 |
| RHD | GUCUCCGGAAACUCGAGGUG | AGG | Exon 2 | chr1: 25,284,622 | 40 |
| F3 | ACAGUGUAGACUUGAUUGAC | GGG | Exon 2 | chr1: 94,540,281 | 41 |
| (CD142) | | | | | |
| B2M | CGUGAGUAAACCUGAAUCUU | TGG | Exon 2 | chr15: 44,715,434 | 42 |
| CIITA | GAUAUUGGCAUAAGCCUCCC | TGG | Exon 3 | chr16: 10,895,747 | 43 |
| TRAC | AGAGUCUCUCAGCUGGUACA | CGG | Exon 1 | chr14: 22,5547,533 | 44 |

In some embodiments, it is within the level of a skilled artisan to identify new loci and/or gRNA targeting sequences for use in methods of genetic disruption to reduce or eliminate expression of a gene as described. For example, for CRISPR/Cas systems, when an existing gRNA targeting sequence for a particular locus (e.g., within a target gene, e.g., set forth in Table 1) is known, an “inch worming” approach can be used to identify additional loci for targeted insertion of transgenes by scanning the flanking regions on either side of the locus for PAM sequences, which usually occurs about every 100 base pairs (bp) across the genome. The PAM sequence will depend on the particular Cas nuclease used because different nucleases usually have different corresponding PAM sequences. The flanking regions on either side of the locus can be between about 500 to 4000 bp long, for example, about 500 bp, about 1000 bp, about 1500 bp, about 2000 bp, about 2500 bp, about 3000 bp, about 3500 bp, or about 4000 bp long. When a PAM sequence is identified within the search range, a new guide can be designed according to the sequence of that locus for use in genetic disruption methods. Although the CRISPR/Cas system is described as illustrative, any gene-editing approaches as described can be used in this method of identifying new loci, including those using ZFNs, TALENS, meganucleases and transposases.

In some embodiments, the cells described herein are made using Transcription Activator-Like Effector Nucleases (TALEN) methodologies. By a “TALE-nuclease” (TALEN) is intended a fusion protein consisting of a nucleic acid-binding domain typically derived from a Transcription Activator Like Effector (TALE) and one nuclease catalytic domain to cleave a nucleic acid target sequence. The catalytic domain can be a nuclease domain and more in embodiments, a domain having endonuclease activity, like for instance I-TevI, ColE7, NucA and Fok-I. In embodiments, the TALE domain can be fused to a meganuclease like for instance I-CreI and I-OnuI or functional variant thereof. In some embodiments, said nuclease is a monomeric TALE-Nuclease. A monomeric TALE-Nuclease is a TALE-Nuclease that does not require dimerization for specific recognition and cleavage, such as the fusions of engineered TAL repeats with the catalytic domain of I-TevI described in WO2012138927. Transcription Activator like Effector (TALE) are proteins from the bacterial species Xanthomonas comprise a plurality of repeated sequences, each repeat comprising di-residues in position 12 and 13 (RVD) that are specific to each nucleotide base of the nucleic acid targeted sequence. Binding domains with similar modular base-per-base nucleic acid binding properties (MBBBD) can also be derived from new modular proteins recently discovered by the applicant in a different bacterial species. The new modular proteins have the advantage of displaying more sequence variability than TAL repeats. In embodiments, RVDs associated with recognition of the different nucleotides are HD for recognizing C. NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In some embodiments, critical amino acids 12 and 13 can be mutated towards other amino acid residues in order to modulate their specificity towards nucleotides A, T, C and G and in to enhance this specificity. TALEN kits are sold commercially.

In some embodiments, the cells are manipulated using zinc finger nuclease (ZFN). A “zinc finger binding protein” is a protein or polypeptide that binds DNA, RNA and/or protein, for example in a sequence-specific manner, as a result of stabilization of protein structure through coordination of a zinc ion. The term zinc finger binding protein is often abbreviated as zinc finger protein or ZFP. The individual DNA binding domains are typically referred to as “fingers.” A ZFP has least one finger, typically two fingers, three fingers, or six fingers. Each finger binds from two to four base pairs of DNA, typically three or four base pairs of DNA. A ZFP binds to a nucleic acid sequence called a target site or target segment. Each finger typically comprises an approximately 30 amino acid, zinc-chelating, DNA-binding subdomain. Studies have demonstrated that a single zinc finger of this class consists of an alpha helix containing the two invariant histidine residues coordinated with zinc along with the two cysteine residues of a single beta turn (see, e.g., Berg & Shi, Science 271:1081-1085 (1996)).

In some embodiments, the cells described herein are made using a homing endonuclease. Such homing endonucleases are well-known to the art (Stoddard 2005). Homing endonucleases recognize a DNA target sequence and generate a single- or double-strand break. Homing endonucleases are highly specific, recognizing DNA target sites ranging from 12 to 45 base pairs (bp) in length, usually ranging from 14 to 40 bp in length. The homing endonuclease may for example correspond to a LAGLIDADG endonuclease, to an HNH endonuclease, or to a GIY-YIG endonuclease. In some embodiments, the homing endonuclease can be an I-CreI variant.

In some embodiments, the cells described herein are made using a meganuclease. Meganucleases are by definition sequence-specific endonucleases recognizing large sequences (Chevalier, B. S, and B. L. Stoddard, Nucleic Acids Res., 2001, 29, 3757-3774). They can cleave unique sites in living cells, thereby enhancing gene targeting by 1000-fold or more in the vicinity of the cleavage site (Puchta et al., Nucleic Acids Res., 1993, 21, 5034-5040: Rouet et al., Mol. Cell. Biol., 1994, 14, 8096-8106: Choulika et al., Mol. Cell. Biol., 1995, 15, 1968-1973; Puchta et al., Proc. Natl. Acad. Sci. USA, 1996, 93, 5055-5060; Sargent et al., Mol. Cell. Biol., 1997, 17, 267-77; Donoho et al., Mol. Cell. Biol, 1998, 18, 4070-4078; Elliott et al., Mol. Cell. Biol., 1998, 18, 93-101; Cohen-Tannoudji et al., Mol. Cell. Biol., 1998, 18, 1444-1448).

In some embodiments, the cells provided herein are made using RNA silencing or RNA interference (RNAi) to knockdown (e.g., decrease, eliminate, or inhibit) the expression of a polypeptide. Useful RNAi methods include those that utilize synthetic RNAi molecules, short interfering RNAs (siRNAs), PIWI-interacting NRAs (piRNAs), short hairpin RNAs (shRNAs), microRNAs (miRNAs), and other transient knockdown methods recognized by those skilled in the art. Reagents for RNAi including sequence specific shRNAs, siRNA, miRNAs and the like are commercially available. For instance, a target polynucleotide, such as any described above, e.g., CIITA. B2M, or NLRC5, can be knocked down in a cell by RNA interference by introducing an inhibitory nucleic acid complementary to a target motif of the target polynucleotide, such as an siRNA, into the cells. In some embodiments, a target polynucleotide, such as any described above, e.g., CIITA, B2M, or NLRC5, can be knocked down in a cell by transducing a shRNA-expressing virus into the cell. In some embodiments, RNA interference is employed to reduce or inhibit the expression of at least one selected from the group consisting of CIITA, B2M, and NLRC5.

### 3. Exemplary Target Polynucleotides and Methods for Reducing Expression

### A. MHC Class I Molecules

In certain embodiments, the modification reduces or eliminates, such as knocks out, the expression of one or more MHC class I molecules (e.g., one or more MHC class I genes encoding one or more MHC class I molecules) by targeting the accessory chain B2M. In some embodiments, the modification occurs using a CRISPR/Cas system. By reducing or eliminating, such as knocking out, expression of B2M, surface trafficking of one or more MHC class I molecules is blocked, and such cells exhibit immune tolerance when engrafted into a recipient subject. In some embodiments, the cell is considered hypoimmunogenic, e.g., in a recipient subject or patient upon administration.

In some embodiments, the target polynucleotide sequence provided herein is a variant of B2M. In some embodiments, the target polynucleotide sequence is a homolog of B2M. In some embodiments, the target polynucleotide sequence is an ortholog of B2M.

In some embodiments, decreased or eliminated expression of B2M reduces or eliminates expression of one or more of the following MHC class I molecules—HLA-A, HLA-B, and HLA-C.

In some embodiments, the engineered cell comprises a modification targeting the B2M gene. In some embodiments, the modification targeting the B2M gene is by using a targeted nuclease system that comprises a Cas protein or a polynucleotide encoding a Cas protein, and at least one guide ribonucleic acid sequence for specifically targeting the B2M gene. In some embodiments, the at least one guide ribonucleic acid sequence (e.g., gRNA targeting sequence) for specifically targeting the B2M gene is selected from the group consisting of SEQ ID NOS:81240-85644 of Appendix 2 or Table 15 of WO2016/183041, the disclosure of which is herein incorporated by reference in its entirety.

In some embodiments, an exogenous nucleic acid or transgene encoding a polypeptide as disclosed herein (e.g., a chimeric antigen receptor, CD47, or another tolerogenic factor disclosed herein) is inserted at the B2M gene. Exemplary transgenes for targeted insertion at the B2M locus include any as described in Section II.B.

Assays to test whether the B2M gene has been inactivated are known and described herein. In some embodiments, the resulting modification of the B2M gene by PCR and the reduction of HLA-I expression can be assays by flow cytometry, such as by FACS analysis. In some embodiments, B2M protein expression is detected using a Western blot of cells lysates probed with antibodies to the B2M protein. In some embodiments, reverse transcriptase polymerase chain reactions (RT-PCR) are used to confirm the presence of the inactivating modification.

In some embodiments, the reduction of the one or more MHC class I molecules expression or function (HLA I when the cells are derived from human cells) in the engineered cells can be measured using techniques known in the art; for example, FACS techniques using labeled antibodies that bind the HLA complex; for example, using commercially available HLA-A, B, C antibodies that bind to the alpha chain of the human major histocompatibility HLA Class I antigens. In addition, the cells can be tested to confirm that the HLA I complex is not expressed on the cell surface. This may be assayed by FACS analysis using antibodies to one or more HLA cell surface components as discussed above. In addition to the reduction of HLA I (or MHC class I), the engineered cells provided herein have a reduced susceptibility to macrophage phagocytosis and NK cell killing. Methods to assay for hypoimmunogenic phenotypes of the engineered cells are described further below.

### B. MHC Class II Molecules

In certain aspects, the modification reduces or eliminates, such as knocks out, the expression of one or more MHC class II molecules by targeting Class II transactivator (CIITA) expression. In some embodiments, the modification occurs using a CRISPR/Cas system. CIITA is a member of the LR or nucleotide binding domain (NBD)leucine-rich repeat (LRR) family of proteins and regulates the transcription of one or more MHC class II genes by associating with the MHC enhanceosome. By reducing or eliminating, such as knocking out, expression of CIITA, expression of one or more MHC class II molecules is reduced thereby also reducing surface expression. In some cases, such cells exhibit immune tolerance when engrafted into a recipient subject. In some embodiments, the cell is considered hypoimmunogenic, e.g., in a recipient subject or patient upon administration.

In some embodiments, the target polynucleotide sequence is a variant of CIITA. In some embodiments, the target polynucleotide sequence is a homolog of CIITA. In some embodiments, the target polynucleotide sequence is an ortholog of CIITA.

In some embodiments, reduced or eliminated expression of CIITA reduces or eliminates expression of one or more of the following MHC class II molecules: HLA-DP. HLA-DM, HLA-DOA, HLA-DOB, HLA-DQ, and HLA-DR.

In some embodiments, the engineered cell comprises a modification targeting the CIITA gene. In some embodiments, the modification targeting the CIITA gene is by a targeted nuclease system that comprises a Cas protein or a polynucleotide encoding a Cas protein, and at least one guide ribonucleic acid sequence for specifically targeting the CIITA gene. In some embodiments, the at least one guide ribonucleic acid sequence (e.g., gRNA targeting sequence) for specifically targeting the CIITA gene is selected from the group consisting of SEQ ID NOS:5184-36352 of Appendix 1 or Table 12 of WO2016183041, the disclosure of which is herein incorporated by reference in its entirety.

In some embodiments, an exogenous nucleic acid or transgene encoding a polypeptide as disclosed herein (e.g., a chimeric antigen receptor, CD47, or another tolerogenic factor disclosed herein) is inserted at the CIITA gene. Exemplary transgenes for targeted insertion at the B2M locus include any as described in Section II.B.

Assays to test whether the CIITA gene has been inactivated are known and described herein. In some embodiments, the resulting modification of the CIITA gene by PCR and the reduction of HLA-II expression can be assays by flow cytometry, such as by FACS analysis. In some embodiments, CIITA protein expression is detected using a Western blot of cells lysates probed with antibodies to the CIITA protein. In some embodiments, reverse transcriptase polymerase chain reactions (RT-PCR) are used to confirm the presence of the inactivating modification.

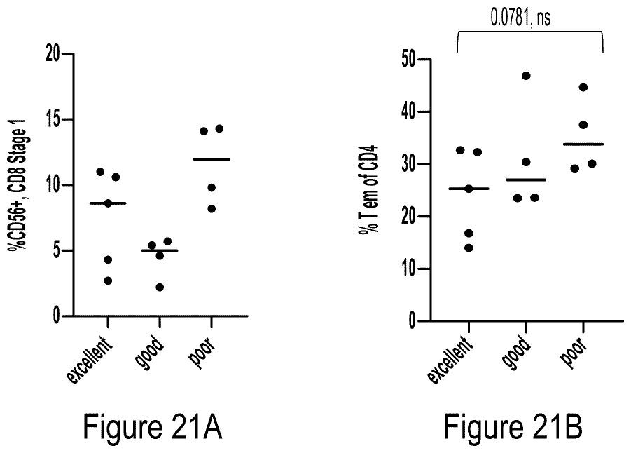

In some embodiments, the reduction of the one or more MHC class II molecules expression or function (HLA II when the cells are derived from human cells) in the engineered cells can be measured using techniques known in the art, such as Western blotting using antibodies to the protein, FACS techniques, RT-PCR techniques, etc. In some embodiments, the engineered cells can be tested to confirm that the HLA II complex is not expressed on the cell surface. Methods to assess surface expression include methods known in the art (See FIG. 21 of WO2018132783, for example) and generally is done using either Western Blots or FACS analysis based on commercial antibodies that bind to human HLA Class II HLA-DR, DP and most DQ antigens. In addition to the reduction of HLA II (or MHC class II), the engineered cells provided herein have a reduced susceptibility to macrophage phagocytosis and NK cell killing. Methods to assay for hypoimmunogenic phenotypes of the engineered cells are described further below.

### B. Overexpression of Polynucleotides

In some embodiments, the engineered cells provided herein are genetically modified or engineered, such as by introduction of one or more modifications into a cell to overexpress a desired polynucleotide in the cell. In some embodiments, the cell to be modified or engineered is an unmodified cell or non-engineered cell that has not previously been introduced with the one or more modifications. In some embodiments, the engineered cells provided herein are genetically modified to include one or more exogenous polynucleotides encoding an exogenous protein (also interchangeably used with the term “transgene”). As described, in some embodiments, the cells are modified to increase expression of certain genes that are tolerogenic (e.g., immune) factors that affect immune recognition and tolerance in a recipient. In some embodiments, the provided engineered cells, such as T cells or NK cells, also express a chimeric antigen receptor (CAR). The one or more polynucleotides, e.g., exogenous polynucleotides, may be expressed (e.g., overexpressed) in the engineered cell together with one or more genetic modifications to reduce expression of a target polynucleotide described in Section I.A above, such as an MHC class I and/or MHC class II molecule. In some embodiments, the provided engineered cells do not trigger or activate an immune response upon administration to a recipient subject.

In some embodiments, the engineered cell includes 1, 2, 3, 4, 5, 6, 7, 8, 9, 10 or more different overexpressed polynucleotides. In some embodiments, the engineered cell includes 1, 2, 3, 4, 5, 6, 7, 8, 9, 10 or more different overexpressed polynucleotides. In some embodiments, the overexpressed polynucleotide is an exogenous polynucleotide. In some embodiments, the engineered cell includes 1, 2, 3, 4, 5, 6, 7, 8, 9, 10 or more different exogenous polynucleotides. In some embodiments, the engineered cell includes 1, 2, 3, 4, 5, 6, 7, 8, 9, 10 or more different exogenous polynucleotides. In some embodiments, the overexpressed polynucleotide is an exogenous polynucleotide that is expressed episomally in the cells. In some embodiments, the overexpressed polynucleotide is an exogenous polynucleotide that is inserted or integrated into one or more genomic loci of the engineered cell.

In some embodiments, expression of a polynucleotide is increased, i.e., the polynucleotide is overexpressed, using a fusion protein containing a DNA-targeting domain and a transcriptional activator. Targeted methods of increasing expression using transactivator domains are known to a skilled artisan.

In some embodiments, the engineered cell contains one or more exogenous polynucleotides in which the one or more exogenous polynucleotides are inserted or integrated into a genomic locus of the cell by non-targeted insertion methods, such as by transduction with a lentiviral vector. In some embodiments, the one or more exogenous polynucleotides are inserted or integrated into the genome of the cell by targeted insertion methods, such as by using homology directed repair (HDR). Any suitable method can be used to insert the exogenous polynucleotide into the genomic locus of the engineered cell by HDR including the gene editing methods described herein (e.g., a CRISPR/Cas system). In some embodiments, the one or more exogenous polynucleotides are inserted into one or more genomic locus, such as any genomic locus described herein (e.g., Table 2). In some embodiments, the exogenous polynucleotides are inserted into the same genomic loci. In some embodiments, the exogenous polynucleotides are inserted into different genomic loci. In some embodiments, the two or more of the exogenous polynucleotides are inserted into the same genomic loci, such as any genomic locus described herein (e.g., Table 2). In some embodiments, two or more exogenous polynucleotides are inserted into a different genomic loci, such as two or more genomic loci as described herein (e.g., Table 2).

In some embodiments, any of gene editing technologies can be used to increase expression of the one or more target polynucleotides or target proteins as described. In some embodiments, the gene editing technology can include systems involving nucleases, integrases, transposases, recombinases. In some embodiments, the gene editing technologies can be used for modifications to increase endogenous gene activity (e.g., by modifying or activating a promoter or enhancer operably linked to a gene). In some embodiments, the gene-editing technologies can be used for knock-in or integration of DNA into a region of the genome (e.g., to introduce a construct encoding the target polynucleotide or target protein, such as a construct encoding any of the tolerogenic factors or any of the other molecules described herein for increased expression in engineered cells). In some embodiments, the gene editing technology mediates single-strand breaks (SSB). In some embodiments, the gene editing technology mediates double-strand breaks (DSB), including in connection with non-homologous end-joining (NHEJ) or homology-directed repair (HDR). In some embodiments, the gene editing technology can include DNA-based editing or prime-editing. In some embodiments, the gene editing technology can include Programmable Addition via Site-specific Targeting Elements (PASTE). Exemplary polynucleotides or overexpression, and methods for overexpressing the same, are described in the following subsections.

### 1. Tolerogenic Factors

In some embodiments, expression of a tolerogenic factor is overexpressed or increased in the cell. It will be understood that embodiments concerning cells modified with respect to expression of a tolerogenic factor may be readily applied to any cell type as described herein, as well as HIP cells. CAR cells, safety switches and other modified/gene edited cells as described herein.

In some embodiments, the engineered cell includes increased expression, i.e., overexpression, of at least one tolerogenic factor. In some embodiments, the tolerogenic factor is any factor that promotes or contributes to promoting or inducing tolerance to the engineered cell by the immune system (e.g., innate or adaptive immune system). In some embodiments, the tolerogenic factor is CD16, CD24, CD35, CD39, CD46, CD47, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, MANF. In some embodiments, the tolerogenic factor is CD47, PD-L1, HLA-E or HLA-G, CCL21, FasL, Serpinb9, CD200 or Mfge8, or any combination thereof. In some embodiments, the cell includes at least one exogenous polynucleotide that includes a polynucleotide that encodes for a tolerogenic factor. For instance, in some embodiments, at least one of the exogenous polynucleotides is a polynucleotide that encodes CD47. Provided herein are cells that do not trigger or activate an immune response upon administration to a recipient subject. As described above, in some embodiments, the cells are modified to increase expression of genes and tolerogenic (e.g., immune) factors that affect immune recognition and tolerance in a recipient.

In some embodiments, the present disclosure provides a cell or population thereof that has been modified to express the tolerogenic factor (e.g., immunomodulatory polypeptide), such as CD47. In some embodiments, the present disclosure provides a method for altering a cell genome to express the tolerogenic factor (e.g., immunomodulatory polypeptide), such as CD47. In some embodiments, the engineered cell expresses an exogenous tolerogenic factor (e.g., immunomodulatory polypeptide), such as an exogenous CD47. In some instances, overexpression or increasing expression of the exogenous polynucleotide is achieved by introducing into the cell (e.g., transducing the cell) with an expression vector comprising a nucleotide sequence encoding a human CD47 polypeptide. In some embodiments, the expression vector may be a viral vector, such as a lentiviral vector) or may be a non-viral vector. In some embodiments, the cell is engineered to contain one or more exogenous polynucleotides in which at least one of the exogenous polynucleotides includes a polynucleotide that encodes for a tolerogenic factor. In some of any embodiments, the tolerogenic factor is CD16, CD24, CD35, CD39, CD46, CD47, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, MANF. In some embodiments, the tolerogenic factor is selected from CD47, PD-L1, HLA-E or HLA-G, CCL21, FasL, Serpinb9, CD200 or Mfge8, or any combination thereof. For instance, in some embodiments, at least one of the exogenous polynucleotides is a polynucleotide that encodes CD47.

In some embodiments, the tolerogenic factor is CD47. In some embodiments, the engineered cell contains an exogenous polynucleotide that encodes CD47, such as human CD47. In some embodiments, CD47 is overexpressed in the cell. In some embodiments, the expression of CD47 is overexpressed or increased in the engineered cell compared to a similar cell of the same cell type that has not been engineered with the modification, such as a reference or unmodified cell, e.g., a cell not engineered with an exogenous polynucleotide encoding CD47. CD47 is a leukocyte surface antigen and has a role in cell adhesion and modulation of integrins. It is normally expressed on the surface of a cell and signals to circulating macrophages not to eat the cell. Useful genomic, polynucleotide and polypeptide information about human CD47 are provided in, for example, the NP_001768.1, NP_942088.1, NM_001777.3 and NM_198793.2.

In some embodiments, the engineered cell includes increased expression. i.e., overexpression, of at least one tolerogenic factor. In some embodiments, the cell includes at least one exogenous polynucleotide that includes a polynucleotide that encodes for a tolerogenic factor. In some embodiments, tolerogenic factors include CD16, CD24, CD35, CD39, CD46, CD47, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, MANF, or any combination thereof. For instance, in some embodiments, at least one of the overexpressed (e.g., exogenous) polynucleotides is a polynucleotide that encodes CD47.

In some embodiments, the present disclosure provides a cell or population thereof that has been modified to express the tolerogenic factor (e.g., immunomodulatory polypeptide), such as CD47. In some embodiments, the present disclosure provides a method for altering a cell genome to express the tolerogenic factor (e.g., immunomodulatory polypeptide), such as CD47. In some embodiments, the engineered cell expresses an exogenous tolerogenic factor (e.g., immunomodulatory polypeptide), such as an exogenous CD47. In some instances, the cell expresses an expression vector comprising a nucleotide sequence encoding a human CD47 polypeptide.

In some embodiments, the engineered cell contains an overexpressed polynucleotide that encodes CD47, such as human CD47. In some embodiments, the engineered cell contains an exogenous polynucleotide that encodes CD47, such as human CD47. In some embodiments, CD47 is overexpressed in the cell. In some embodiments, the expression of CD47 is increased in the engineered cell compared to a similar reference or unmodified cell (including with any other modifications) except that the reference or unmodified cell does not include the exogenous polynucleotide encoding CD47, CD47 is a leukocyte surface antigen and has a role in cell adhesion and modulation of integrins. It is normally expressed on the surface of a cell and signals to circulating macrophages not to eat the cell. Useful genomic, polynucleotide and polypeptide information about human CD47 are provided in, for example, the NP_001768.1, NP 942088.1, NM_001777.3 and NM_198793.2.

In some embodiments, the cell outlined herein comprises an exogenous nucleotide sequence encoding a CD47 polypeptide has at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the cell outlined herein comprises an exogenous nucleotide sequence encoding a CD47 polypeptide having an amino acid sequence as set forth in NCBI Ref. Sequence Nos. NP_001768.1 and NP_942088.1. In some embodiments, the cell comprises an exogenous nucleotide sequence for CD47 having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_001777.3 and NM_198793.2. In some embodiments, the cell comprises an exogenous nucleotide sequence for CD47 as set forth in NCBI Ref. Sequence Nos. NM_001777.3 and NM_198793.2.

In some embodiments, the cell outlined herein comprises an exogenous nucleotide sequence encoding a CD47 polypeptide has at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the cell outlined herein comprises an exogenous nucleotide sequence encoding a CD47 polypeptide having an amino acid sequence as set forth in NCBI Ref. Sequence Nos. NP_001768.1 and NP_942088.1. In some embodiments, the cell comprises an exogenous nucleotide sequence for CD47 having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_001777.3 and NM_198793.2. In some embodiments, the cell comprises an exogenous nucleotide sequence for CD47 as set forth in NCBI Ref. Sequence Nos. NM_001777.3 and NM_198793.2.

In some embodiments, the cell comprises an exogenous CD47 polypeptide having at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the cell outlined herein comprises an exogenous CD47 polypeptide having an amino acid sequence as set forth in NCBI Ref. Sequence Nos. NP_001768.1 and NP_942088.1.

In some embodiments, the cell comprises an overexpressed polynucleotide encoding a CD47 polypeptide having at least 95% sequence identity (e.g., 95%, 96%, 97%, 98%, 99%, or more) to an amino acid sequence as set forth in SEQ ID NO: 1. In some embodiments, the cell comprises an exogenous polynucleotide encoding a CD47 polypeptide having at least 95% sequence identity (e.g., 95%, 96%, 97%, 98%, 99%, or more) to an amino acid sequence as set forth in SEQ ID NO: 1. In some embodiments, the cell comprises an overexpressed polynucleotide encoding a CD47 polypeptide having the amino acid sequence as set forth in SEQ ID NO: 1. In some embodiments, the cell comprises an exogenous polynucleotide encoding a CD47 polypeptide having the amino acid sequence as set forth in SEQ ID NO: 1.

In some embodiments, the cell comprises an overexpressed CD47 polypeptide having at least 95% sequence identity (e.g., 95%, 96%, 97%, 98%, 99%, or more) to an amino acid sequence as set forth in SEQ ID NO: 2. In some embodiments, the cell comprises an exogenous CD47 polypeptide having at least 95% sequence identity (e.g., 95%, 96%, 97%, 98%, 99%, or more) to an amino acid sequence as set forth in SEQ ID NO: 2. In some embodiments, the cell comprises an overexpressed CD47 polypeptide having the amino acid sequence as set forth in SEQ ID NO: 2. In some embodiments, the cell comprises an exogenous CD47 polypeptide having the amino acid sequence as set forth in SEQ ID NO: 2. In some embodiments, an exogenous polynucleotide encoding CD47 is integrated into the genome of the cell by targeted or non-targeted methods of insertion, such as described further below. In some embodiments, targeted insertion is by homology-dependent insertion into a target loci, such as by insertion into any one of the gene loci depicted in Table 2, e.g., a B2M gene, a CIITA gene, a TRAC gene, a TRBC gene. In some embodiments, targeted insertion is by homology-independent insertion, such as by insertion into a safe harbor locus. In some cases, the polynucleotide encoding CD47 is inserted into a safe harbor locus, such as but not limited to, a gene locus selected from AAVS1, CCR5, CLYBL, ROSA26, and SHS231. In embodiments, the polynucleotide encoding CD47 is inserted into the CCR5 gene locus, the PPP1R12C (also known as AAVS1) gene locus or the CLYBL gene locus.

In some embodiments, all or a functional portion of CD47 can be linked to other components such as a signal peptide, a leader sequence, a secretory signal, a label (e.g., a reporter gene), or any combination thereof. In some embodiments, the nucleic acid sequence encoding a signal peptide of CD47 is replaced with a nucleic acid sequence encoding a signal peptide from a heterologous protein. The heterologous protein can be, for example, CD8u, CD28, tissue plasminogen activator (tPA), growth hormone, granulocyte-macrophage colony stimulating factor (GM-CSF), GM-CSF receptor (GM-CSFRa), or an immunoglobulin (e.g., IgE or IgK). In some embodiments, the signal peptide is a signal peptide from an immunoglobulin (such as IgG heavy chain or IgG-kappa light chain), a cytokine (such as interleukin-2 (IL-2), or CD33), a serum albumin protein (e.g., HSA or albumin), a human azurocidin preprotein signal sequence, a luciferase, a trypsinogen (e.g., chymotrypsinogen or trypsinogen) or other signal peptide able to efficiently express a protein by or on a cell.

In certain embodiments, the exogenous polynucleotide encoding CD47 is operably linked to a promoter.

In some embodiments, the exogenous polynucleotide encoding CD47 is inserted into any one of the gene loci depicted in Table 2. In some cases, the exogenous polynucleotide encoding CD47 is inserted into a safe harbor locus, such as but not limited to, a gene locus selected from AAVS1, CCR5, CLYBL, ROSA26, and SHS231. In embodiments, the exogenous polynucleotide encoding CD47 is inserted into the CCR5 gene locus, the PPP1R12C (also known as AAVS1) gene locus or the CLYBL gene locus. In some embodiments, the exogenous polynucleotide encoding CD47 is inserted into a B2M gene locus or a CIITA gene locus. In some embodiments, the engineered cell is a T cell and the exogenous polynucleotide encoding CD47 is inserted into a TRAC gene locus, or a TRBC gene locus. In some embodiments, a suitable gene editing system (e.g., CRISPR/Cas system or any of the gene editing systems described herein) is used to facilitate the insertion of a polynucleotide encoding CD47, into a genomic locus of the cell.

In some embodiments, CD47 protein expression is detected using a Western blot of cell lysates probed with antibodies against the CD47 protein. In some embodiments, reverse transcriptase polymerase chain reactions (RT-PCR) are used to confirm the presence of the exogenous CD47 mRNA.

In some embodiments, the engineered cell contains an exogenous polynucleotide that encodes CD200, such as human CD200. In some embodiments, CD200 is overexpressed in the cell. In some embodiments, the expression of CD200 is increased in the engineered cell compared to a similar reference or unmodified cell (including with any other modifications) except that the reference or unmodified cell does not include the exogenous polynucleotide encoding CD200. Useful genomic, polynucleotide and polypeptide information about human CD200 are provided in, for example, the GeneCard Identifier GC03P112332, HGNC No. 7203, NCBI Gene ID 4345, Uniprot No. P41217, and NCBI RefSeq Nos. NP_001004196.2, NM_001004196.3, NP_001305757.1, NM_001318828.1, NP 005935.4, NM_005944.6, XP_005247539.1, and XM_005247482.2. In certain embodiments, the polynucleotide encoding CD200 is operably linked to a promoter.

In some embodiments, the polynucleotide encoding CD200 is inserted into any one of the gene loci depicted in Table 2. In some cases, the polynucleotide encoding CD200 is inserted into a safe harbor locus, such as but not limited to, a gene locus selected from an AAVS1 (also known as PPP1R12C), ABO, CCR5, CLYBL, CXCR4, F3 (also known as CD142), FUT1, HMGB1, KDM5D, LRP1 (also known as CD91), MICA, MICB, RHD, ROSA26, and SHS231 gene locus. In embodiments, the polynucleotide encoding CD200 is inserted into the CCR5 gene locus, the PPP1R12C (also known as AAVS1) gene locus or the CLYBL gene locus. In some embodiments, the polynucleotide encoding CD200 is inserted into a B2M gene locus or a CIITA gene locus. In some embodiments, the engineered cell is a T cell and the polynucleotide encoding CD200 is inserted into a TRAC gene locus, or a TRBC gene locus. In some embodiments, a suitable gene editing system (e.g., CRISPR/Cas system or any of the gene editing systems described herein) is used to facilitate the insertion of a polynucleotide encoding CD200, into a genomic locus of the cell.

In some embodiments, CD200 protein expression is detected using a Western blot of cell lysates probed with antibodies against the CD200 protein. In some embodiments, reverse transcriptase polymerase chain reactions (RT-PCR) are used to confirm the presence of the exogenous CD200 mRNA.

In some embodiments, the engineered cell contains an exogenous polynucleotide that encodes HLA-E, such as human HLA-E. In some embodiments, HLA-E is overexpressed in the cell. In some embodiments, the expression of HLA-E is increased in the engineered cell compared to a similar reference or unmodified cell (including with any other modifications) except that the reference or unmodified cell does not include the exogenous polynucleotide encoding HLA-E. Useful genomic, polynucleotide and polypeptide information about human HLA-E are provided in, for example, the GeneCard Identifier GC06P047281, HGNC No. 4962, NCBI Gene ID 3133, Uniprot No. P13747, and NCBI RefSeq Nos. NP_005507.3 and NM_005516.5. In certain embodiments, the polynucleotide encoding HLA-E is operably linked to a promoter.

In some embodiments, the polynucleotide encoding HLA-E is inserted into any one of the gene loci depicted in Table 2. In some cases, the polynucleotide encoding HLA-E is inserted into a safe harbor locus, such as but not limited to, a gene locus selected from an AAVS1 (also known as PPP1R12C), ABO, CCR5, CLYBL, CXCR4, F3 (also known as CD142), FUT1, HMGB1, KDM5D, LRP1 (also known as CD91), MICA, MICB, RHD, ROSA26, and SHS231 gene locus. In embodiments, the polynucleotide encoding HLA-E is inserted into the CCR5 gene locus, the PPP1R12C (also known as AAVS1) gene locus or the CLYBL gene locus. In some embodiments, the polynucleotide encoding HLA-E is inserted into a B2M gene locus or a CIITA gene locus. In some embodiments, the engineered cell is a T cell and the polynucleotide encoding HLA-E is inserted into a TRAC gene locus, or a TRBC gene locus. In some embodiments, a suitable gene editing system (e.g., CRISPR/Cas system or any of the gene editing systems described herein) is used to facilitate the insertion of a polynucleotide encoding HLA-E, into a genomic locus of the cell.

In some embodiments, HLA-E protein expression is detected using a Western blot of cell lysates probed with antibodies against the HLA-E protein. In some embodiments, reverse transcriptase polymerase chain reactions (RT-PCR) are used to confirm the presence of the exogenous HLA-E mRNA.

In some embodiments, the engineered cell contains an exogenous polynucleotide that encodes HLA-G, such as human HLA-G. In some embodiments, HLA-G is overexpressed in the cell. In some embodiments, the expression of HLA-G is increased in the engineered cell compared to a similar reference or unmodified cell (including with any other modifications) except that the reference or unmodified cell does not include the exogenous polynucleotide encoding HLA-G. Useful genomic, polynucleotide and polypeptide information about human HLA-G are provided in, for example, the GeneCard Identifier GC06P047256, HGNC No. 4964, NCBI Gene ID 3135, Uniprot No. P17693, and NCBI RefSeq Nos. NP_002118.1 and NM_002127.5. In certain embodiments, the polynucleotide encoding HLA-G is operably linked to a promoter.

In some embodiments, the polynucleotide encoding HLA-G is inserted into any one of the gene loci depicted in Table 2. In some cases, the polynucleotide encoding HLA-G is inserted into a safe harbor locus, such as but not limited to, a gene locus selected from an AAVS1 (also known as PPP1R12C), ABO, CCR5, CLYBL, CXCR4, F3 (also known as CD142), FUT1, HMGB1, KDM5D, LRP1 (also known as CD91), MICA, MICB. RHD, ROSA26, and SHS231 gene locus. In embodiments, the polynucleotide encoding HLA-G is inserted into the CCR5 gene locus, the PPP1R12C (also known as AAVS1) gene locus or the CLYBL gene locus. In some embodiments, the polynucleotide encoding HLA-G is inserted into a B2M gene locus or a CIITA gene locus. In some embodiments, the engineered cell is a T cell and the polynucleotide encoding HLA-G is inserted into a TRAC gene locus, or a TRBC gene locus. In some embodiments, a suitable gene editing system (e.g., CRISPR/Cas system or any of the gene editing systems described herein) is used to facilitate the insertion of a polynucleotide encoding HLA-G, into a genomic locus of the cell.

In some embodiments, HLA-G protein expression is detected using a Western blot of cell lysates probed with antibodies against the HLA-G protein. In some embodiments, reverse transcriptase polymerase chain reactions (RT-PCR) are used to confirm the presence of the exogenous HLA-G mRNA.

In some embodiments, the engineered cell contains an exogenous polynucleotide that encodes PD-L1, such as human PD-L1. In some embodiments, PD-L1 is overexpressed in the cell. In some embodiments, the expression of PD-L1 is increased in the engineered cell compared to a similar reference or unmodified cell (including with any other modifications) except that the reference or unmodified cell does not include the exogenous polynucleotide encoding PD-L1. Useful genomic, polynucleotide and polypeptide information about human PD-L1 or CD274 are provided in, for example, the GeneCard Identifier GC09P005450, HGNC No. 17635, NCBI Gene ID 29126, Uniprot No. Q9NZQ7, and NCBI RefSeq Nos. NP_001254635.1. NM_001267706.1, NP_054862.1, and NM_014143.3. In certain embodiments, the polynucleotide encoding PD-L1 is operably linked to a promoter.

In some embodiments, the polynucleotide encoding PD-L1 is inserted into any one of the gene loci depicted in Table 2. In some cases, the polynucleotide encoding PD-L1 is inserted into a safe harbor locus, such as but not limited to, a gene locus selected from an AAVS1 (also known as PPP1R12C), ABO, CCR5, CLYBL, CXCR4, F3 (also known as CD142), FUT1, HMGB1, KDM5D, LRP1 (also known as CD91), MICA, MICB, RHD, ROSA26, and SHS231 gene locus. In embodiments, the polynucleotide encoding PD-L1 is inserted into the CCR5 gene locus, the PPP1R12C (also known as AAVS1) gene locus or the CLYBL gene locus. In some embodiments, the polynucleotide encoding PD-L1 is inserted into a B2M gene locus, or a CIITA gene locus. In some embodiments, the engineered cell is a T cell and the polynucleotide encoding PD-L1 is inserted into a TRAC gene locus, or a TRBC gene locus. In some embodiments, a suitable gene editing system (e.g., CRISPR/Cas system or any of the gene editing systems described herein) is used to facilitate the insertion of a polynucleotide encoding PD-L1, into a genomic locus of the cell.

In some embodiments, PD-L1 protein expression is detected using a Western blot of cell lysates probed with antibodies against the PD-L1 protein. In some embodiments, reverse transcriptase polymerase chain reactions (RT-PCR) are used to confirm the presence of the exogenous PD-L1 mRNA.

In some embodiments, the engineered cell contains an exogenous polynucleotide that encodes FasL, such as human FasL. In some embodiments, FasL is overexpressed in the cell. In some embodiments, the expression of FasL is increased in the engineered cell compared to a similar reference or unmodified cell (including with any other modifications) except that the reference or unmodified cell does not include the exogenous polynucleotide encoding FasL. Useful genomic, polynucleotide and polypeptide information about human Fas ligand (which is known as FasL, FASLG, CD178, TNFSF6, and the like) are provided in, for example, the GeneCard Identifier GC01P172628, HGNC No. 11936, NCBI Gene ID 356, Uniprot No. P48023, and NCBI RefSeq Nos. NP 000630.1, NM_000639.2, NP_001289675.1, and NM_001302746.1. In certain embodiments, the polynucleotide encoding Fas-L is operably linked to a promoter.

In some embodiments, the polynucleotide encoding Fas-L is inserted into any one of the gene loci depicted in Table 2. In some cases, the polynucleotide encoding Fas-L is inserted into a safe harbor locus, such as but not limited to, a gene locus selected from an AAVS1 (also known as PPP1R12C), ABO, CCR5, CLYBL, CXCR4, F3 (also known as CD142), FUT, HMGB1, KDM5D, LRP1 (also known as CD91), MICA, MICB, RHD, ROSA26, and SHS231 gene locus. In embodiments, the polynucleotide encoding Fas-L is inserted into the CCR5 gene locus, the PPP1R12C (also known as AAVS1) gene locus or the CLYBL gene locus. In some embodiments, the polynucleotide encoding Fas-L is inserted into a B2M gene locus or a CIITA gene locus. In some embodiments, the engineered cell is a T cell and the polynucleotide encoding Fas-L is inserted into a TRAC gene locus, or a TRBC gene locus. In some embodiments, a suitable gene editing system (e.g., CRISPR/Cas system or any of the gene editing systems described herein) is used to facilitate the insertion of a polynucleotide encoding Fas-L, into a genomic locus of the cell.

In some embodiments, Fas-L protein expression is detected using a Western blot of cell lysates probed with antibodies against the Fas-L protein. In some embodiments, reverse transcriptase polymerase chain reactions (RT-PCR) are used to confirm the presence of the exogenous Fas-L mRNA.

In some embodiments, the engineered cell contains an exogenous polynucleotide that encodes CCL21, such as human CCL21. In some embodiments, CCL21 is overexpressed in the cell. In some embodiments, the expression of CCL21 is increased in the engineered cell compared to a similar reference or unmodified cell (including with any other modifications) except that the reference or unmodified cell does not include the exogenous polynucleotide encoding CCL21. Useful genomic, polynucleotide and polypeptide information about human CCL21 are provided in, for example, the GeneCard Identifier GC09M034709, HGNC No. 10620, NCBI Gene ID 6366, Uniprot No. 000585, and NCBI RefSeq Nos. NP_002980.1 and NM_002989.3. In certain embodiments, the polynucleotide encoding CCL21 is operably linked to a promoter.

In some embodiments, the polynucleotide encoding CCL21 is inserted into any one of the gene loci depicted in Table 2. In some cases, the polynucleotide encoding CCL21 is inserted into a safe harbor locus, such as but not limited to, a gene locus selected from an AAVS1 (also known as PPP1R12C), ABO, CCR5, CLYBL, CXCR4, F3 (also known as CD142), FUT1, HMGB1, KDM5D, LRP1 (also known as CD91), MICA, MICB, RHD, ROSA26, and SHS231 gene locus. In embodiments, the polynucleotide encoding CCL21 is inserted into the CCR5 gene locus, the PPP1R12C (also known as AAVS1) gene locus or the CLYBL gene locus. In some embodiments, the polynucleotide encoding CCL21 is inserted into a B2M gene locus or a CIITA gene locus. In some embodiments, the engineered cell is a T cell and the polynucleotide encoding CCL21 is inserted into a TRAC gene locus, or a TRBC gene locus. In some embodiments, a suitable gene editing system (e.g., CRISPR/Cas system or any of the gene editing systems described herein) is used to facilitate the insertion of a polynucleotide encoding CCL21, into a genomic locus of the cell.

In some embodiments, CCL21 protein expression is detected using a Western blot of cell lysates probed with antibodies against the CCL21 protein. In some embodiments, reverse transcriptase polymerase chain reactions (RT-PCR) are used to confirm the presence of the exogenous CCL21 mRNA.

In some embodiments, the engineered cell contains an exogenous polynucleotide that encodes CCL22, such as human CCL22. In some embodiments, CCL22 is overexpressed in the cell. In some embodiments, the expression of CCL22 is increased in the engineered cell compared to a similar reference or unmodified cell (including with any other modifications) except that the reference or unmodified cell does not include the exogenous polynucleotide encoding CCL22. Useful genomic, polynucleotide and polypeptide information about human CCL22 are provided in, for example, the GeneCard Identifier GC16P057359, HGNC No. 10621, NCBI Gene ID 6367, Uniprot No. 000626, and NCBI RefSeq Nos. NP_002981.2, NM_002990.4, XP_016879020.1, and XM_017023531.1. In certain embodiments, the polynucleotide encoding CCL22 is operably linked to a promoter.

In some embodiments, the polynucleotide encoding CCL22 is inserted into any one of the gene loci depicted in Table 2. In some cases, the polynucleotide encoding CCL22 is inserted into a safe harbor locus, such as but not limited to, a gene locus selected from an AAVS1 (also known as PPP1R12C), ABO, CCR5, CLYBL, CXCR4, F3 (also known as CD142), FUT1, HMGB1, KDM5D, LRP1 (also known as CD91), MICA, MICB, RHD, ROSA26, and SHS231 gene locus. In embodiments, the polynucleotide encoding CCL22 is inserted into the CCR5 gene locus, the PPP1R12C (also known as AAVS) gene locus or the CLYBL gene locus. In some embodiments, the polynucleotide encoding CCL22 is inserted into a B2M gene locus or a CIITA gene locus. In some embodiments, the engineered cell is a T cell and the polynucleotide encoding CCL22 is inserted into a TRAC gene locus, or a TRBC gene locus. In some embodiments, a suitable gene editing system (e.g., CRISPR/Cas system or any of the gene editing systems described herein) is used to facilitate the insertion of a polynucleotide encoding CCL22, into a genomic locus of the cell.

In some embodiments, CCL22 protein expression is detected using a Western blot of cell lysates probed with antibodies against the CCL22 protein. In some embodiments, reverse transcriptase polymerase chain reactions (RT-PCR) are used to confirm the presence of the exogenous CCL22 mRNA.

In some embodiments, the engineered cell contains an exogenous polynucleotide that encodes Mfge8, such as human Mfge8. In some embodiments, Mfge8 is overexpressed in the cell. In some embodiments, the expression of Mfge8 is increased in the engineered cell compared to a similar reference or unmodified cell (including with any other modifications) except that the reference or unmodified cell does not include the exogenous polynucleotide encoding Mfge8. Useful genomic, polynucleotide and polypeptide information about human Mfge8 are provided in, for example, the GeneCard Identifier GC15M088898, HGNC No. 7036, NCBI Gene ID 4240, Uniprot No. Q08431, and NCBI RefSeq Nos. NP_001108086.1, NM_001114614.2, NP_001297248.1, NM_001310319.1, NP_001297249.1, NM_001310320.1. NP_001297250.1, NM_001310321.1, NP_005919.2, and NM_005928.3. In certain embodiments, the polynucleotide encoding Mfge8 is operably linked to a promoter.

In some embodiments, the polynucleotide encoding Mfge8 is inserted into any one of the gene loci depicted in Table 2. In some cases, the polynucleotide encoding Mfge8 is inserted into a safe harbor locus, such as but not limited to, a gene locus selected from an AAVS1 (also known as PPP1R12C), ABO, CCR5, CLYBL, CXCR4, F3 (also known as CD142), FUT1, HMGB1, KDM5D, LRP1 (also known as CD91), MICA, MICB, RHD, ROSA26, and SHS231 gene locus. In embodiments, the polynucleotide encoding Mfge8 is inserted into the CCR5 gene locus, the PPP1R12C (also known as AAVS1) gene locus or the CLYBL gene locus. In some embodiments, the polynucleotide encoding Mfge8 is inserted into a B2M gene locus or a CIITA gene locus. In some embodiments, the engineered cell is a T cell and the polynucleotide encoding Mfge8 is inserted into a TRAC gene locus, or a TRBC gene locus. In some embodiments, a suitable gene editing system (e.g., CRISPR/Cas system or any of the gene editing systems described herein) is used to facilitate the insertion of a polynucleotide encoding Mfge8, into a genomic locus of the cell.

In some embodiments, Mfge8 protein expression is detected using a Western blot of cell lysates probed with antibodies against the Mfge8 protein. In some embodiments, reverse transcriptase polymerase chain reactions (RT-PCR) are used to confirm the presence of the exogenous Mfge8 mRNA.

In some embodiments, the engineered cell contains an exogenous polynucleotide that encodes SerpinB9, such as human SerpinB9. In some embodiments, SerpinB9 is overexpressed in the cell. In some embodiments, the expression of SerpinB9 is increased in the engineered cell compared to a similar reference or unmodified cell (including with any other modifications) except that the reference or unmodified cell does not include the exogenous polynucleotide encoding SerpinB9. Useful genomic, polynucleotide and polypeptide information about human SerpinB9 are provided in, for example, the GeneCard Identifier GC06M002887, HGNC No. 8955, NCBI Gene ID 5272, Uniprot No. P50453, and NCBI RefSeq Nos. NP_004146.1. NM_004155.5, XP_005249241.1, and XM_005249184.4. In certain embodiments, the polynucleotide encoding SerpinB9 is operably linked to a promoter.

In some embodiments, the polynucleotide encoding SerpinB9 is inserted into any one of the gene loci depicted in Table 2. In some cases, the polynucleotide encoding SerpinB9 is inserted into a safe harbor locus, such as but not limited to, a gene locus selected from an AAVS1 (also known as PPP1R12C), ABO. CCR5, CLYBL, CXCR4, F3 (also known as CD142), FUT1, HMGB1, KDM5D, LRP1 (also known as CD91), MICA, MICB, RHD, ROSA26, and SHS231 gene locus. In embodiments, the polynucleotide encoding SerpinB9 is inserted into the CCR5 gene locus, the PPP1R12C (also known as AAVS1) gene locus or the CLYBL gene locus. In some embodiments, the polynucleotide encoding SerpinB9 is inserted into a B2M gene locus or a CIITA gene locus. In some embodiments, the engineered cell is a T cell and the polynucleotide encoding SerpinB9 is inserted into a TRAC gene locus, or a TRBC gene locus. In some embodiments, a suitable gene editing system (e.g., CRISPR/Cas system or any of the gene editing systems described herein) is used to facilitate the insertion of a polynucleotide encoding SerpinB9, into a genomic locus of the cell.

In some embodiments, SerpinB9 protein expression is detected using a Western blot of cell lysates probed with antibodies against the SerpinB9 protein. In some embodiments, reverse transcriptase polymerase chain reactions (RT-PCR) are used to confirm the presence of the exogenous SerpinB9 mRNA.

### 2. Chimeric Antigen Receptor

In some embodiments, a provided engineered cell is further modified to express a chimeric antigen receptor (CAR). It will be understood that embodiments concerning CAR modified cells may be readily applied to any suitable cell type as described herein, as well as HIP cells, safety switches and other modified/gene edited cells as described herein.

In some embodiments, a provided cell contains a genetic modification of one or more target polynucleotide sequences that regulates the expression of one or more MHC class I molecules, one or more MHC class II molecules, or one or more MHC class I molecules and one or more MHC class II molecules overexpresses a tolerogenic factor as described herein (e.g., CD47), and expresses a CAR. In some embodiments, the cell is one in which: B2M is reduced or eliminated (e.g., knocked out), CIITA is reduced or eliminated (e.g., knocked out), CD47 is overexpressed, and a CAR is expressed. In some embodiments, the cell is B2^{-/-}, CIITA^{-/-}, CD47tg, CAR+. In some embodiments, the cell (e.g., T cell) may additional be one in which TRAC is reduced or eliminated (e.g., knocked out). In some embodiments, the cell is B2^{-/-}, CIITA, CD47tg.TRAC^{-/-} CAR+.

In some embodiments, a polynucleotide encoding a CAR is introduced into the cell. In some embodiments, the cell is a T cell, such as a primary T cell or a T cell differentiated from a pluripotent cell (e.g., iPSC). In some embodiments, the cell is a Natural Killer (NK) cell, such as a primary NK cell or an NK cell differentiated from a pluripotent cell (e.g., iPSC).

In some embodiments, the CAR is selected from the group consisting of a first generation CAR, a second generation CAR, a third generation CAR, and a fourth generation CAR. In some embodiments, the CAR is or comprises a first generation CAR comprising an antigen binding domain, a transmembrane domain, and at least one signaling domain (e.g., one, two or three signaling domains). In some embodiments, the CAR comprises a second generation CAR comprising an antigen binding domain, a transmembrane domain, and at least two signaling domains. In some embodiments, the CAR comprises a third generation CAR comprising an antigen binding domain, a transmembrane domain, and at least three signaling domains. In some embodiments, a fourth generation CAR comprising an antigen binding domain, a transmembrane domain, three or four signaling domains, and a domain which upon successful signaling of the CAR induces expression of a cytokine gene. In some embodiments, the antigen binding domain is or comprises an antibody, an antibody fragment, an scFv or a Fab.

In some embodiments, any one of the cells described herein comprises a nucleic acid encoding a CAR or a first generation CAR. In some embodiments, a first generation CAR comprises an antigen binding domain, a transmembrane domain, and signaling domain. In some embodiments, a signaling domain mediates downstream signaling during T cell activation.

In some embodiments, any one of the cells described herein comprises a nucleic acid encoding a CAR or a second generation CAR. In some embodiments, a second generation CAR comprises an antigen binding domain, a transmembrane domain, and two signaling domains. In some embodiments, a signaling domain mediates downstream signaling during T cell activation. In some embodiments, a signaling domain is a costimulatory domain. In some embodiments, a costimulatory domain enhances cytokine production, CAR T cell proliferation, and/or CAR T cell persistence during T cell activation.

In some embodiments, any one of the cells described herein comprises a nucleic acid encoding a CAR or a third generation CAR. In some embodiments, a third generation CAR comprises an antigen binding domain, a transmembrane domain, and at least three signaling domains. In some embodiments, a signaling domain mediates downstream signaling during T cell activation. In some embodiments, a signaling domain is a costimulatory domain. In some embodiments, a costimulatory domain enhances cytokine production. CAR T cell proliferation, and or CAR T cell persistence during T cell activation. In some embodiments, a third generation CAR comprises at least two costimulatory domains. In some embodiments, the at least two costimulatory domains are not the same.

In some embodiments, any one of the cells described herein comprises a nucleic acid encoding a CAR or a fourth generation CAR. In some embodiments, a fourth generation CAR comprises an antigen binding domain, a transmembrane domain, and at least two, three, or four signaling domains. In some embodiments, a signaling domain mediates downstream signaling during T cell activation. In some embodiments, a signaling domain is a costimulatory domain. In some embodiments, a costimulatory domain enhances cytokine production. CAR T cell proliferation, and or CAR T cell persistence during T cell activation.

In some embodiments, an engineered cell provided herein (e.g., primary or iPSC-derived T cell or primary or iPSC-derived NK cell) includes a polynucleotide encoding a CAR, wherein the polynucleotide is inserted in a genomic locus. In some embodiments, the polynucleotide is inserted into a safe harbor locus, such as but not limited to, an AAVS1, CCR5, CLYBL, ROSA26, SHS231, F3 (also known as CD142), MICA, MICB, LRP1 (also known as CD91), HMGB1, ABO, RHD, FUT1, or KDM5D gene locus. In some embodiments, the polynucleotide is inserted in a B2M, CIITA, TRAC, TRB, PD1 or CTLA4 gene. Any suitable method can be used to insert the CAR into the genomic locus of the hypoimmunogenic cell including the gene editing methods described herein (e.g., a CRISPR/Cas system).

In some embodiments, a first, second, third, or fourth generation CAR further comprises a domain which upon successful signaling of the CAR induces expression of a cytokine gene. In some embodiments, a cytokine gene is endogenous or exogenous to a target cell comprising a CAR which comprises a domain which upon successful signaling of the CAR induces expression of a cytokine gene. In some embodiments, a cytokine gene encodes a pro-inflammatory cytokine. In some embodiments, a cytokine gene encodes IL-1, IL-2, IL-9, IL-12, IL-18, TNF, or IFN-gamma, or functional fragment thereof. In some embodiments, a domain which upon successful signaling of the CAR induces expression of a cytokine gene is or comprises a transcription factor or functional domain or fragment thereof. In some embodiments, a domain which upon successful signaling of the CAR induces expression of a cytokine gene is or comprises a transcription factor or functional domain or fragment thereof. In some embodiments, a transcription factor or functional domain or fragment thereof is or comprises a nuclear factor of activated T cells (NFAT), an NF-kB, or functional domain or fragment thereof. See, e.g., Zhang. C. et al., Engineering CAR-T cells. Biomarker Research. 5:22 (2017); WO 2016126608; Sha, H. et al. Chimaeric antigen receptor T-cell therapy for tumour immunotherapy. Bioscience Reports Jan. 27, 2017, 37 (1).

A skilled artisan is familiar with CARs and different components and configurations of CARs. Any known CAR can be employed in connection with the provided embodiments. In addition to the CARs described herein, various CARs and nucleotide sequences encoding the same are known in the art and would be suitable for engineering cells as described herein. See, e.g., WO2013040557: WO2012079000; WO2016030414; Smith T, et al., Nature Nanotechnology. 2017. DOI: 10.1038/NNANO.2017.57, the disclosures of which are herein incorporated by reference. Exemplary features and components of a CAR are described in the following subsections.

### A. Antigen Binding Domain

In some embodiments, a CAR antigen binding domain (ABD) is or comprises an antibody or antigen-binding portion thereof. In some embodiments, a CAR antigen binding domain is or comprises an scFv or Fab.

In some embodiments, an antigen binding domain binds to a cell surface antigen of a cell. In some embodiments, a cell surface antigen is characteristic of (e.g., expressed by) a particular or specific cell type. In some embodiments, a cell surface antigen is characteristic of more than one type of cell.

In some embodiments, the antigen may be an antigen that is exclusively or preferentially expressed on tumor cells, or an antigen that is characteristic of an autoimmune or inflammatory disease. In some embodiments, the antigen binding domain (ABD) targets an antigen characteristic of a neoplastic cell. For instance, the antigen binding domain targets an antigen expressed by a neoplastic or cancer cell. In some embodiments, the ABD binds a tumor associated antigen. In some embodiments, the antigen characteristic of a neoplastic cell (e.g., antigen associated with a neoplastic or cancer cell) or a tumor associated antigen is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor.

In some embodiments, the target antigen is an antigen that includes, but is not limited to, Epidermal Growth Factor Receptors (EGFR) (including ErbB1/EGFR, ErbB2/HER2, ErbB3/HER3, and ErbB4/HER4), Fibroblast Growth Factor Receptors (FGFR) (including FGF1, FGF2, FGF3, FGF4, FGF5, FGF6, FGF7, FGF18, and FGF21) Vascular Endothelial Growth Factor Receptors (VEGFR) (including VEGF-A, VEGF-B, VEGF-C, VEGF-D, and PIGF), RET Receptor and the Eph Receptor Family (including EphA1, EphA2, EphA3, EphA4, EphA5, EphA6, EphA7, EphA8, EphA9, EphA10, EphB1, EphB2, EphB3, EphB4, and EphB6), CXCR1, CXCR2, CXCR3, CXCR4, CXCR6, CCR1, CCR2, CCR3, CCR4, CCR5, CCR6, CCR8, CFTR, CIC-1, CIC-2, CIC-4, CIC-5, CIC-7, C1C-Ka, C1C-Kb, Bestrophins, TMEM16A, GABA receptor, glycin receptor, ABC transporters, NAV1.1, NAV1.2, NAV1.3, NAV1.4, NAV1.5, NAV1.6, NAV1.7, NAV1.8, NAV1.9, sphingosin-1-phosphate receptor (S1P1R), NMDA channel, transmembrane protein, multispan transmembrane protein, T-cell receptor motifs; T-cell alpha chains; T-cell β chains; T-cell γ chains: T-cell δ chains, CCR7, CD3, CD4, CD5, CD7, CD8, CD11b, CD11c, CD16, CD19, CD20, CD21, CD22, CD25, CD28, CD34, CD35, CD40, CD45RA, CD45RO, CD52, CD56, CD62L, CD68, CD80, CD95, CD117, CD127, CD133, CD137 (4-1 BB), CD163, F4/80, IL-4Ra, Sca-1, CTLA-4, GITR, GARP, LAP, granzyme B, LFA-1, transferrin receptor, NKp46, perforin, CD4+, Th1, Th2, Th7, Th40, Th22, Th9, Tfh, Canonical Treg, FoxP3+, Tr1, Th3, Treg17, T_{REG}, CDCP, NT5E, EpCAM, CEA, gpA33, Mucins, TAG-72, Carbonic anhydrase IX, PSMA, Folate binding protein, Gangliosides (e.g., CD2, CD3, GM2), Lewis-γ², VEGF, VEGFR 1/2/3, αVβ3, α5β1, ErbB1/EGFR, ErbB1/HER2, ErB3, c-MET, IGF1R, EphA3, TRAIL-R1, TRAIL-R2, RANKL, FAP, Tenascin, PDL-1, BAFF, HDAC, ABL, FLT3, KIT, MET, RET, IL-1β, ALK, RANKL, mTOR, CTLA-4, IL-6, IL-6R, JAK3, BRAF, PTCH, Smoothened, PIGF, ANPEP, TIMP1, PLAUR, PTPRJ, LTBR, or ANTXR1, Folate receptor alpha (FRa), ERBB2 (Her2/neu), EphA2, IL-13Ra2, epidermal growth factor receptor (EGFR), Mesothelin, TSHR, CD19, CD123, CD22, CD30, CD171, CS-1, CLL-1, CD33, EGFRvIII, GD2, GD3, BCMA, MUC16 (CA125), LiCAM, LeY, MSLN, IL13Rα1, L1-CAM, Tn Ag, prostate specific membrane antigen (PSMA), ROR1, FLT3, FAP, TAG72, CD38, CD44v6, CEA, EPCAM, B7H3, KIT, interleukin-11 receptor a (IL-11Ra), PSCA, PRSS21, VEGFR2, LewisY, CD24, platelet-derived growth factor receptor-beta (PDGFR-beta), SSEA-4, CD20, MUC1, NCAM, Prostase, PAP, ELF2M, Ephrin B2, IGF-1 receptor, CAIX, LMP2, gplOO, bcr-abl, tyrosinase, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, ETV6-AML, sperm protein 17, XAGE1, Tie 2, MAD-CT-1, MAD-CT-2, Major histocompatibility complex class I-related gene protein (MR1), urokinase-type plasminogen activator receptor (uPAR), Fos-related antigen 1, p53, p53 mutant, prostein, survivin, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYPIB I, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLECl2A, BST2, EMR2, LY75, GPC3, FCRL5, IGLL1, a neoantigen, CD133, CD15, CD184, CD24, CD56, CD26, CD29, CD44, HLA-A, HLA-B, HLA-C, (HLA-A,B,C) CD49f, CD151 CD340, CD200, tkrA, trkB, or trkC, or an antigenic fragment or antigenic portion thereof.

In some embodiments, exemplary target antigens include, but are not limited to, CDS, CD19, CD20, CD22, CD23, CD30, CD70, Kappa, Lambda, and B cell maturation agent (BCMA) (associated with leukemias); CS1/SLAMF7, CD38, CD138, GPRC5D, TACI, and BCMA (associated with myelomas); GD2, HER2, EGFR, EGFRvIII, B7H3, PSMA, PSCA, CAIX, CD171, CEA, CSPG4, EPHA2, FAP, FRa, IL-13Ra, Mesothelin, MUC1, MUC16, and ROR1 (associated with solid tumors).

In some embodiments, the CAR is a CD19 CAR. In some embodiments, the extracellular binding domain of the CD19 CAR comprises an antibody that specifically binds to CD19, for example, human CD19. In some embodiments, the extracellular binding domain of the CD19 CAR comprises an scFv antibody fragment derived from the FMC63 monoclonal antibody (FMC63), which comprises the heavy chain variable region (VH) and the light chain variable region (VL) of FMC63 connected by a linker peptide. In some embodiments, the linker peptide is a “Whitlow” linker peptide. FMC63 and the derived scFv have been described in Nicholson et al., Mal. immun. 34(16-17):1157-1165 (1997) and PCT Application Publication No. WO2018/213337 A 1, the entire content of each of which is incorporated by reference herein.

In some embodiments, the extracellular binding domain of the CD19 CAR comprises an antibody derived from one of the CD19-specific antibodies including, for example, SJ25C1 (Bejcek et al., Cancer Res. 55:2346-2351 (1995)), HD37 (Pezutto et al., J. Immunol. 138(9):2793-2799 (1987)), 4G7 (Meeker et al., Hybridoma 3:305-320 (1984)), B43 (Bejcek (1995)), BLY3 (Bejcek (1995)), B4 (Freedman et al., 70:418-427 (1987)), B4 HB12b (Kansas & Tedder, J. Immunol. 147:4094-4102 (1991); Yazawa et al., Proc. Natl. Acad. Sci. USA 102:15178-15183 (2005); Herbst et al., J. Pharmacol. Exp. Ther. 335:213-222 (2010)), BU12 (Gallard et al., J. Immunology, 148(10); 2983-2987 (1992)), and CLB-CD19 (De Rie Cell. Immunol. 118:368-381(1989)).

In some embodiments, the CAR is CD22 CAR. CD22, which is a transmembrane protein found mostly on the surface of mature B cells that functions as an inhibitory receptor for B cell receptor (BCR) signaling. CD22 is expressed in 60-70% of B cell lymphomas and leukemias (e.g., B-chronic lymphocytic leukemia, hairy cell leukemia, acute lymphocytic leukemia (ALL), and Burkitt's lymphoma) and is not present on the cell surface in early stages of B cell development or on stem cells. In some embodiments, the CD22 CAR comprises an extracellular binding domain that specifically binds CD22, a transmembrane domain, an intracellular signaling domain, and/or an intracellular costimulatory domain. In some embodiments, the extracellular binding domain of the CD22 CAR comprises an scFv antibody fragment derived from the m971 monoclonal antibody (m971), which comprises the heavy chain variable region (VH) and the light chain variable region (VL) of m971 connected by a linker. In some embodiments, the extracellular binding domain of the CD22 CAR comprises an scFv antibody fragment derived from m971-L7, which an affinity matured variant of m971 with significantly improved CD22 binding affinity compared to the parental antibody m971 (improved from about 2 nM to less than 50 pM). In some embodiments, the scFv antibody fragment derived from m971-L7 comprises the VH and the VL of m971-L7 connected by a 3×G4S linker. In some embodiments, the extracellular binding domain of the CD22 CAR comprises immunotoxins HA22 or BL22. Immunotoxins BL22 and HA22 are therapeutic agents that comprise an scFv specific for CD22 fused to a bacterial toxin, and thus can bind to the surface of the cancer cells that express CD22 and kill the cancer cells. BL22 comprises a dsFv of an anti-CD22 antibody, RFB4, fused to a 38-kDa truncated form of Pseudomonas exotoxin A (Bang et al., Clin. Cancer Res., 11:1545-50 (2005)). HA22 (CAT8015, moxetumomab pasudotox) is a mutated, higher affinity version of BL22 (Ho et al., J. Biol. Chem., 280(1); 607-17 (2005)). Suitable sequences of antigen binding domains of HA22 and BL22 specific to CD22 are disclosed in, for example, U.S. Pat. Nos. 7,541,034; 7,355.012; and 7,982,011, which are hereby incorporated by reference in their entirety.

In some embodiments, the CAR is BCMA CAR. BCMA is a tumor necrosis family receptor (TNFR) member expressed on cells of the B cell lineage, with the highest expression on terminally differentiated B cells or mature B lymphocytes. BCMA is involved in mediating the survival of plasma cells for maintaining long-term humoral immunity. The expression of BCMA has been recently linked to a number of cancers, such as multiple myeloma, Hodgkin's and non-Hodgkin's lymphoma, various leukemias, and glioblastoma. In some embodiments, the BCMA CAR comprises an extracellular binding domain that specifically binds BCMA, a transmembrane domain, an intracellular signaling domain, and/or an intracellular costimulatory domain. In some embodiments, the extracellular binding domain of the BCMA CAR comprises an antibody that specifically binds to BCMA, for example, human BCMA. CARs directed to BCMA have been described in PCT Application Publication Nos. WO2016/014789, WO2016/014565. WO2013/154760, and WO 2015/128653. BCMA-binding antibodies are also disclosed in PCT Application Publication Nos. WO2015/166073 and WO2014/068079. In some embodiments, the extracellular binding domain of the BCMA CAR comprises an scFv antibody fragment derived from a murine monoclonal antibody as described in Carpenter et al., Clin. Cancer Res. 19(8):2048-2060 (2013). In some embodiments, the scFv antibody fragment is a humanized version of the murine monoclonal antibody (Sommermeyer et al., Leukemia 31:2191-2199 (2017)). In some embodiments, the extracellular binding domain of the BCMA CAR comprises single variable fragments of two heavy chains (VHH) that can bind to two epitopes of BCMA as described in Zhao et al., J. Hematol. Oneal. 11(1):141 (2018). In some embodiments, the extracellular binding domain of the BCMA CAR comprises a fully human heavy-chain variable domain (FHVH) as described in Lam et al., Nat. Commun. 11(1):283 (2020).

In some embodiments, the antigen binding domain targets an antigen characteristic of an autoimmune or inflammatory disorder. In some embodiments, the ABD binds an antigen associated with an autoimmune or inflammatory disorder. In some instances, the antigen is expressed by a cell associated with an autoimmune or inflammatory disorder. In some embodiments, the autoimmune or inflammatory disorder is selected from chronic graft-vs-host disease (GVHD), lupus, arthritis, immune complex glomerulonephritis, goodpasture, uveitis, hepatitis, systemic sclerosis or scleroderma, type I diabetes, multiple sclerosis, cold agglutinin disease, Pemphigus vulgaris, Grave's disease, autoimmune hemolytic anemia, Hemophilia A, Primary Sjogren's Syndrome, thrombotic thrombocytopenia purrpura, neuromyelits optica, Evan's syndrome, IgM mediated neuropathy, cyroglobulinemia, dermatomyositis, idiopathic thrombocytopenia, ankylosing spondylitis, bullous pemphigoid, acquired angioedema, chronic urticarial, antiphospholipid demyelinating polyneuropathy, and autoimmune thrombocytopenia or neutropenia or pure red cell aplasias, while exemplary non-limiting examples of alloimmune diseases include allosensitization (see, for example, Blazar et al., 2015, Am. J. Transplant, 15(4):931-41) orxenosensitization from hematopoietic or solid organ transplantation, blood transfusions, pregnancy with fetal allosensitization, neonatal alloimmune thrombocytopenia, hemolytic disease of the newborn, sensitization to foreign antigens such as can occur with replacement of inherited or acquired deficiency disorders treated with enzyme or protein replacement therapy, blood products, and gene therapy. Allosensitization, in some instances, refers to the development of an immune response (such as circulating antibodies) against human leukocyte antigens that the immune system of the recipient subject or pregnant subject considers to be non-self antigens. In some embodiments, the antigen characteristic of an autoimmune or inflammatory disorder is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor.

In some embodiments, an antigen binding domain of a CAR binds to a ligand expressed on B cells, plasma cells, or plasmablasts. In some embodiments, an antigen binding domain of a CAR binds to CD10, CD19, CD20, CD22, CD24, CD27, CD38, CD45R, CD138, CD319, BCMA, CD28, TNF, interferon receptors, GM-CSF, ZAP-70, LFA-1, CD3 gamma, CD5 or CD2. See, US 2003/0077249: WO 2017/058753; WO 2017/058850, the contents of which are herein incorporated by reference. In some embodiments, the CAR is an anti-CD19 CAR. In some embodiments, the CAR is an anti-BCMA CAR.

In some embodiments, the antigen binding domain targets an antigen characteristic of senescent cells, e.g., urokinase-type plasminogen activator receptor (uPAR). In some embodiments, the ABD binds an antigen associated with a senescent cell. In some instances, the antigen is expressed by a senescent cell. In some embodiments, the CAR may be used for treatment or prophylaxis of disorders characterized by the aberrant accumulation of senescent cells, e.g., liver and lung fibrosis, atherosclerosis, diabetes and osteoarthritis.

In some embodiments, the antigen binding domain targets an antigen characteristic of an infectious disease. In some embodiments, the ABD binds an antigen associated with an infectious disease. In some instances, the antigen is expressed by a cell affected by an infectious disease. In some embodiments, wherein the infectious disease is selected from HIV, hepatitis B virus, hepatitis C virus, Human herpes virus, Human herpes virus 8 (HHV-8, Kaposi sarcoma-associated herpes virus (KSHV)), Human T-lymphotrophic virus-1 (HTLV-1). Merkel cell polyomavirus (MCV), Simian virus 40 (SV40), Epstein-Barr virus, CMV, human papillomavirus. In some embodiments, the antigen characteristic of an infectious disease is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, HIV Env, gpl20, or CD4-induced epitope on HIV-1 Env.

In any of these embodiments, the extracellular binding domain of the CAR can be codon-optimized for expression in a host cell or have variant sequences to increase functions of the extracellular binding domain.

In some embodiments, the CAR is bispecific to two target antigens. In some embodiments, the target antigens are different target antigens. In some of any such embodiments, the two different target antigens are any two different antigens described above. In some embodiments, the extracellular binding domains are different and bind two different antigens from (i) CD19 and CD20, (ii) CD20 and L1-CAM, (iii) L1-CAM and GD2, (iv) EGFR and L1-CAM, (v) CD19 and CD22, (vi) EGFR and C-MET, (vii) EGFR and HER2, (viii)C-MET and HER2, or (ix) EGFR and ROR1. In some embodiments, each of the two different antigen binding domains is an scFv. In some embodiments, the C-terminus of one variable domain (VH or VL) of a first scFv is tethered to the N-terminus of the second scFv (VL or VH, respectively) via a polypeptide linker. In some embodiments, the linker connects the N-terminus of the VH with the C-terminus of VL or the C-terminus of VH with the N-terminus of VL. These scFvs lack the constant regions (Fc) present in the heavy and light chains of the native antibody. The scFvs, specific for at least two different antigens, are arranged in tandem and linked to the co-stimulatory domain and the intracellular signaling domain via a transmembrane domain. In some embodiments, an extracelluar spacer domain may be linked between the antigen-specific binding region and the transmembrane domain.

In some embodiments, each antigen-specific targeting region of the CAR comprises a divalent (or bivalent) single-chain variable fragment (di-scFvs, bi-scFvs). In CARs comprising di-scFVs, two scFvs specific for each antigen are linked together by producing a single peptide chain with two VH and two VL regions, yielding tandem scFvs. (Xiong, Cheng-Yi; Natarajan, A; Shi. X B; Denardo, G L; Denardo. S J (2006). “Development of tumor targeting anti-MUC-1 multimer: effects of di-scFv unpaired cysteine location on PEGylation and tumor binding”. Protein Engineering Design and Selection 19 (8): 359-367; Kufer, Peter; Lutterbüse, Ralf; Baeuerle, Patrick A. (2004). “A revival of bispecific antibodies”. Trends in Biotechnology 22 (5); 238-244). CARs comprising at least two antigen-specific targeting regions would express two scFvs specific for each of the two antigens. The resulting antigen-specific targeting region, specific for at least two different antigens, is joined to the co-stimulatory domain and the intracellular signaling domain via a transmembrane domain. In some embodiments, an extracelluar spacer domain may be linked between the antigen-specific binding domain and the transmembrane domain.

In some embodiments, each antigen-specific targeting region of the CAR comprises a diabody. In a diabody, the scFvs are created with linker peptides that are too short for the two variable regions to fold together, driving the scFvs to dimerize. Still shorter linkers (one or two amino acids) lead to the formation of trimers, the so-called triabodies or tribodies. Tetrabodies may also be used.

In some embodiments, the cell is engineered to express more than one CAR, such as two different CARs, in which each CAR has an antigen-binding domain directed to a different target antigen. In some of any such embodiments, the two different target antigens are any two different antigens described above. In some embodiments, the extracellular binding domains are different and bind two different antigens from (i) CD19 and CD20, (ii) CD20 and L1-CAM, (iii) L1-CAM and GD2, (iv) EGFR and L1-CAM, (v) CD19 and CD22, (vi) EGFR and C-MET, (vii) EGFR and HER2, (viii)C-MET and HER2, or (ix) EGFR and ROR1.

In some embodiments, two different engineered cells are prepared that contain the provided modifications with each engineered with a different CAR In some embodiments, each of the two different CARs has an antigen-binding domain directed to a different target antigen. In some of any such embodiments, the two different target antigens are any two different antigens described above. In some embodiments, the extracellular binding domains are different and bind two different antigens from (i) CD19 and CD20, (ii) CD20 and L1-CAM, (iii) L1-CAM and GD2, (iv) EGFR and L1-CAM, (v) CD19 and CD22, (vi) EGFR and C-MET, (vii) EGFR and HER2. (viii)C-MET and HER2, or (ix) EGFR and ROR1. In some embodiments, a population of engineered cells (e.g., hypoimmunogenic) expressing a first CAR directed against a first target antigen and a population of engineered cells (e.g., hypoimmunogenic) expressing a second CAR directed against a second target antigen are separately administered to the subject. In some embodiments, the first and second population of cells are administered sequentially in any order. For instance, the population of cells expressing the second CAR is administered a after administration of the population of cells expressing the first CAR.

### B. Spacer

In some embodiments, the CAR further comprises one or more spacers, e.g., wherein the spacer is a first spacer between the antigen binding domain and the transmembrane domain. In some embodiments, the first spacer includes at least a portion of an immunoglobulin constant region or variant or modified version thereof. In some embodiments, the spacer is a second spacer between the transmembrane domain and a signaling domain. In some embodiments, the second spacer is an oligopeptide, e.g., wherein the oligopeptide comprises glycine and serine residues such as but not limited to glycine-serine doublets. In some embodiments, the CAR comprises two or more spacers, e.g., a spacer between the antigen binding domain and the transmembrane domain and a spacer between the transmembrane domain and a signaling domain.

### C. Transmembrane Domain

In some embodiments, the CAR transmembrane domain comprises at least a transmembrane region of the alpha, beta or zeta chain of a T cell receptor, CD28, CD3 epsilon, CD45, CD4, CD5, CD8, CD9, CD16, CD22, CD28, CD33, CD37, CD64, CD80, CD86, CD134, CD137, CD154, or functional variant thereof. In some embodiments, the transmembrane domain comprises at least a transmembrane region(s) of CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or functional variant thereof.

### D. Signaling Domain(S)

In some embodiments, a CAR described herein comprises one or at least one signaling domain selected from one or more of B7-1/CD80; B7-2/CD86: B7-H1/PD-L1: B7-H2; B7-H3; B7-H4; B7-H6: B7-H7; BTLA/CD272; CD28; CTLA-4: Gi24NISTA/B7-H5; ICOS/CD278; PD-1; PD-L2/B7-DC; PDCD6); 4-1BB/TNFSF9/CD137; 4-1BB Ligand/TNFSF9: BAFF/BLyS/TNFSF13B; BAFF R/TNFRSF13C; CD27/TNFRSF7; CD27 Ligand/TNFSF7: CD30/TNFRSF8; CD30 Ligand/TNFSF8; CD40/TNFRSF5; CD40/TNFSF5; CD40 Ligand/TNFSF5; DR3/TNFRSF25; GITR/TNFRSF18; GITR Ligand/TNFSF18; HVEM/TNFRSF14: LIGHT/TNFSF14; Lymphotoxin-alpha/TNF-beta; OX40/TNFRSF4; OX40 Ligand/TNFSF4; RELT/TNFRSF19L; TAC1/TNFRSF13B; TL1A/TNFSF15; TNF-alpha; TNF RII/TNFRSF1B); 2B4/CD244/SLAMF4; BLAME/SLAMF8; CD2; CD2F-10/SLAMF9; CD48/SLAMF2; CD58/LFA-3; CD84/SLAMF5; CD229/SLAMF3; CRACC/SLAMF7; NTB-A/SLAMF6; SLAM/CD150); CD2; CD7; CD53; CD82/Kai-1; CD90/Thy1; CD96; CD160; CD200; CD300a/LMIR1; HLA Class I; HLA-DR; Ikaros; Integrin alpha 4/CD49d; Integrin alpha 4 beta 1; Integrin alpha 4 beta 7/LPAM-1; LAG-3; TCL1A; TCL1B; CRTAM; DAP12; Dectin-1/CLEC7A; DPPIV/CD26; EphB6; TIM-1/KIM-1/HAVCR; TIM-4; TSLP; TSLP R; lymphocyte function associated antigen-1 (LFA-1); NKG2C, a CD3 zeta domain, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, or functional fragment thereof.

In some embodiments, the at least one signaling domain comprises a CD3 zeta domain or an immunoreceptor tyrosine-based activation motif (ITAM), or functional variant thereof.

In some embodiments, a CAR comprises a signaling domain which is a costimulatory domain. In some embodiments, a CAR comprises a second costimulatory domain. In some embodiments, a CAR comprises at least two costimulatory domains. In some embodiments, a CAR comprises at least three costimulatory domains. In some embodiments, a CAR comprises a costimulatory domain selected from one or more of CD27, CD28, 4-IBB. CD134/OX40, CD30, CD40, PD-1, ICOS, lymphocyte function-associated antigen-1 (LFA-1), CD2, CD7, LIGHT, NKG2C, B7-H3, a ligand that specifically binds with CD83. In some embodiments, if a CAR comprises two or more costimulatory domains, two costimulatory domains are different. In some embodiments, if a CAR comprises two or more costimulatory domains, two costimulatory domains are the same.

In other embodiments, the at least one signaling domain comprises (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the at least one signaling domain comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least one signaling domain comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the at least two signaling domains comprise a CD3 zeta domain or an immunoreceptor tyrosine-based activation motif (ITAM), or functional variant thereof. In other embodiments, the at least two signaling domains comprise (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the at least one signaling domain comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least two signaling domains comprise a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the at least three signaling domains comprise a CD3 zeta domain or an immunoreceptor tyrosine-based activation motif (ITAM), or functional variant thereof. In other embodiments, the at least three signaling domains comprise (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the least three signaling domains comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least three signaling domains comprise a (i) a CD3 zeta domain, or an immunoreceptor tyrosine-based activation motif (ITAM), or functional variant thereof: (ii) a CD28 domain or functional variant thereof; (iii) a 4-1BB domain, or a CD134 domain, or functional variant thereof; and (iv) a cytokine or costimulatory ligand transgene.

In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof.

In some embodiments, the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof.

In some embodiments, the CAR comprises (i) a CD3 zeta domain, or an immunoreceptor tyrosine-based activation motif (ITAM), or functional variant thereof; (ii) a CD28 domain, or a 4-1BB domain, or functional variant thereof, and/or (iii) a 4-1BB domain, or a CD134 domain, or functional variant thereof.

In some embodiments, the CAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

### E. Exemplary Cars

In some embodiments, the CAR comprises an extracellular antigen binding domain (e.g., antibody or antibody fragment, such as an scFv) that binds to an antigen (e.g., tumor antigen), a spacer (e.g., containing a hinge domain, such as any as described herein), a transmembrane domain (e.g., any as described herein), and an intracellular signaling domain (e.g., any intracellular signaling domain, such as a primary signaling domain or costimulatory signaling domain as described herein). In some embodiments, the intracellular signaling domain is or includes a primary cytoplasmic signaling domain. In some embodiments, the intracellular signaling domain additionally includes an intracellular signaling domain of a costimulatory molecule (e.g., a costimulatory domain). Any of such components can be any as described above.

Examples of exemplary components of a CAR are described in Table 3. In provided aspects, the sequences of each component in a CAR can include any combination listed in Table 3.

TABLE 3
CAR components and Exemplary Sequences

| Component | | |
|---|---|---|
| Extracellular binding domain | Sequence | SEQ ID NO |
| Anti-CD19 scFv (FMC63) | DIQMTQTTSSLSASLGDRVTISCRASQDISKY | 22 |
| | LNWYQQKPDGTVKLLIYHTSRLHSGVPSRFS | |
| | GSGSGTDYSLTISNLEQEDIATYFCQQGNTLP | |
| | YTFGGGTKLEITGSTSGSGKPGSGEGSTKGE | |
| | VKLQESGPGLVAPSQSLSVTCTVSGVSLPDY | |
| | GVSWIRQPPRKGLEWLGVIWGSETTYYNSA | |
| | LKSRLTIIKDNSKSQVFLKMNSLQTDDTAIYY | |
| | CAKHYYYGGSYAMDYWGQGTSVTVSS | |
| Anti-CD19 scFv (FMC63) | DIQMTQTTSSLSASLGDRVTISCRASQDISKYLN | 23 |
| | WYQQKPDGTVKLLIYHTSRLHSGVPSRFSGSGSG | |
| | TDYSLTISNLEQEDIATYFCQQGNTLPYTFGGGT | |
| | KLEITGGGGSGGGGSGGGGSEVKLQESGPGLVA | |
| | PSQSLSVTCTVSGVSLPDYGVSWIRQPPRKGLEW | |
| | LGVIWGSETTYYNSALKSRLTIIKDNSKSQVFLK | |
| | MNSLQTDDTAIYYCAKHYYYGGSYAMDYWGQ | |
| | GTSVTVSS | |

Spacer (e.g., hinge)

| | | |
|---|---|---|
| IgG4 Hinge | ESKYGPPCPPCP | 24 |
| CD8 Hinge | TTTPAPRPPTPAPTIASQPLSLRPE | 25 |
| CD28 | IEVMYPPPYLDNEKSNGTIIHVKGKHLCPSPLFPG | 26 |
| | PSKP | |

Transmembrane

| | | |
|---|---|---|
| CD8 | ACRPAAGGAVHTRGLDFACDIYIWAPLAGTCGV | 27 |
| | LLLSLVITLYC | |
| CD28 | FWVLVVVGGVLACYSLLVTVAFIIFWV | 28 |
| CD28 | FWVLVVVGGVLACYSLLVTVAFIIFWV | 29 |

Costimulatory domain

| | | |
|---|---|---|
| CD28 | RSKRSRLLHSDYMNMTPRRPGPTRKHYQPYAPP | 30 |
| | RDFAAYRS | |
| 4-1BB | KRGRKKLLYIFKQPFMRPVQTTQEEDGCSCRFPE | 31 |
| | EEEGGCEL | |

Primary Signaling Domain

| | | |
|---|---|---|
| CD3zeta | RVKFSRSADAPAYQQGQNQLYNELNLGRREEYD | 33 |
| | VLDKRRGRDPEMGGKPRRKNPQEGLYNELQKD | |
| | KMAEAYSEIGMKGERRRGKGHDGLYQGLSTAT | |
| | KDTYDALHMQALPPR | |
| CD3zeta | RVKFSRSADAPAYKQGQNQLYNELNLGRREEYD | 34 |
| | VLDKRRGRDPEMGGKPRRKNPQEGLYNELQKD | |
| | KMAEAYSEIGMKGERRRGKGHDGLYQGLSTAT | |
| | KDTYDALHMQALPPR | |

CARs (also known as chimeric immunoreceptors, chimeric T cell receptors, or artificial T cell receptors) are receptor proteins that have been engineered to give host cells (e.g., T cells) the new ability to target a specific protein. The receptors are chimeric because they combine both antigen-binding and T cell activating functions into a single receptor. A CAR may comprise an extracellular binding domain (also referred to as a “binder”) that specifically binds a target antigen, a transmembrane domain, and an intracellular signaling domain. In certain embodiments, the CAR may further comprise one or more additional elements, including one or more signal peptides, one or more extracellular hinge domains, and/or one or more intracellular costimulatory domains. Domains may be directly adjacent to one another, or there may be one or more amino acids linking the domains. The nucleotide sequence encoding a CAR may be derived from a mammalian sequence, for example, a mouse sequence, a primate sequence, a human sequence, or combinations thereof. In the cases where the nucleotide sequence encoding a CAR is non-human, the sequence of the CAR may be humanized. The nucleotide sequence encoding a CAR may also be codon-optimized for expression in a mammalian cell, for example, a human cell. In any of these embodiments, the nucleotide sequence encoding a CAR may be at least 80% identical (e.g., at least 80%, at least 85%, at least 90%, at least 95%, at least 96%, at least 97%, at least 98%, at least 99%, or 100% identical) to any of the nucleotide sequences disclosed herein. The sequence variations may be due to codon-optimalization, humanization, restriction enzyme-based cloning scars, and/or additional amino acid residues linking the functional domains, etc.

In certain embodiments, the CAR may comprise a signal peptide at the N-terminus. Non-limiting examples of signal peptides include CD8α signal peptide. IgK signal peptide, and granulocyte-macrophage colony-stimulating factor receptor subunit alpha (GMCSFR-α, also known as colony stimulating factor 2 receptor subunit alpha (CSF2RA)) signal peptide, and variants thereof, the amino acid sequences of which are provided in Table 4 below.

TABLE 4
Exemplary sequences of signal peptides

| SEQ ID NO: | Sequence | Description |
|---|---|---|
| 6A | MALPVTALLLPLALLLHAARP | CD8α signal peptide |
| 7A | METDTLLLWVLLLWVPGSTG | IgK signal peptide |
| 8A | MLLLVTSLLLCELPHPAFLLIP | GMCSFR-α (CSF2RA) |
| | | signal peptide |

In certain embodiments, the extracellular binding domain of the CAR may comprise one or more antibodies specific to one target antigen or multiple target antigens. The antibody may be an antibody fragment, for example, an scFv, or a single-domain antibody fragment, for example, a VHH. In certain embodiments, the scFv may comprise a heavy chain variable region (V_{H}) and a light chain variable region (V_{L}) of an antibody connected by a linker. The V_{H} and the V_{L} may be connected in either order, i.e., V_{H}-linker-V_{L} or V_{L}-linker-V_{H}. Non-limiting examples of linkers include Whitlow linker, (G4S). (n can be a positive integer, e.g., 1, 2, 3, 4, 5, 6, etc.) linker, and variants thereof. In certain embodiments, the antigen may be an antigen that is exclusively or preferentially expressed on tumor cells, or an antigen that is characteristic of an autoimmune or inflammatory disease. Exemplary target antigens include, but are not limited to, CD5, CD19, CD20, CD22, CD23, CD30, CD33, CD70, Kappa, Lambda, B cell maturation agent (BCMA), and G-protein coupled receptor family C group 5 member D (GPRC5D) (associated with leukemias): CS1/SLAMF7, CD38, CD138, GPRC5D, TACI, and BCMA (associated with myelomas); CD123, LeY, NKG2D ligand, and WT1 (associated with other hematological cancers); GD2, HER2, EGFR, EGFRvIII, B7H3, PSMA, PSCA, CAIX, CD171, CEA, CSPG4, EPHA2, FAP, FRα, IL-13Rα, Mesothelin, MUC1, MUC16, ROR1, C-Met, CD133, Ep-CAM, GPC3, HPV16-E6, IL13Ra2, MAGEA3, MAGEA4, MART1, NY-ESO-1, VEGFR2, α-Folate receptor, CD24, CD44v7/8, EGP-2, EGP-40, erb-B2, erb-B 2,3,4, FBP, Fetal acethylcholine e receptor, GD2, GD3, HMW-MAA, IL-11Rα, KDR, Lewis Y, L1-cell adhesion molecule, MAGE-A1, Oncofetal antigen (h5T4), and TAG-72 (associated with solid tumors): A*02 (associated with organ transplantation); fibroblast activation protein (FAP)(associated with fibrosis); urokinase-type plasminogen activator receptor (uPAR) (associated with senescence). In certain embodiments, the CAR can be re-engineered as a chimeric autoantibody receptor (CAAR) to selectively deplete autoreactive immune cells. In certain embodiments, CAARs are engineered to target autoantibodies present on immune cells. Exemplary target antigens for CAARs include, but are not limited to, DSG3 (associated with pemphigus volgaris); factor VIII (FVIII)(associated with haemophilia). In any of these embodiments, the extracellular binding domain of the CAR can be codon-optimized for expression in a host cell or have variant sequences to increase functions of the extracellular binding domain.

In certain embodiments, the CAR may comprise a hinge domain, also referred to as a spacer. The terms “hinge” and “spacer” may be used interchangeably in the present disclosure. Non-limiting examples of hinge domains include CD8α hinge domain, CD28 hinge domain, IgG4 hinge domain, IgG4 hinge-CH2-CH3 domain, and variants thereof, the amino acid sequences of which are provided in Table 5 below.

TABLE 5
Exemplary sequences of hinge domains

| SEQ ID NO: | Sequence | Description |
|---|---|---|
| 9A | TTTPAPRPPTPAPTIASQPLSLRPEACRPAA | CD8α hinge domain |
| | GGAVHTRGLDFACD | |
| 10A | IEVMYPPPYLDNEKSNGTIIHVKGKHLCPSP | CD28 hinge domain |
| | LFPGPSKP | |
| 11A | AAAIEVMYPPPYLDNEKSNGTIIHVKGKHL | CD28 hinge domain |
| | CPSPLFPGPSKP | |
| 12A | ESKYGPPCPPCP | IgG4 hinge domain |
| 13A | ESKYGPPCPSCP | IgG4 hinge domain |
| 14A | ESKYGPPCPPCPAPEFLGGPSVFLFPPKPKD | IgG4 hinge-CH2-CH3 |
| | TLMISRTPEVTCVVVDVSQEDPEVQFNWY | domain |
| | VDGVEVHNAKTKPREEQFNSTYRVVSVLT | |
| | VLHQDWLNGKEYKCKVSNKGLPSSIEKTIS | |
| | KAKGQPREPQVYTLPPSQEEMTKNQVSLT | |
| | CLVKGFYPSDIAVEWESNGQPENNYKTTPP | |
| | VLDSDGSFFLYSRLTVDKSRWQEGNVFSCS | |
| | VMHEALHNHYTQKSLSLSLGK | |

In certain embodiments, the transmembrane domain of the CAR may comprise a transmembrane region of the alpha, beta, or zeta chain of a T cell receptor, CD28, CD3e, CD45, CD4, CD5, CD8, CD9, CD16, CD22, CD33, CD37, CD64, CD80, CD86, CD134, CD137, CD154, or a functional variant thereof, including the human versions of each of these sequences. In other embodiments, the transmembrane domain may comprise a transmembrane region of CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40UL/CD154, VEGFR2, FAS, and FGFR2B, or a functional variant thereof, including the human versions of each of these sequences. Table 6 provides the amino acid sequences of a few exemplary transmembrane domains.

TABLE 6
Exemplary sequences of transmembrane domains

| SEQ ID NO: | Sequence | Description |
|---|---|---|
| 15A | IYIWAPLAGTCGVLLLSLVITLYC | CD8α transmembrane domain |
| 16A | FWVLVVVGGVLACYSLLVTVAFIIF | CD28 transmembrane domain |
| | WV | |
| 17A | MFWVLVVVGGVLACYSLLVTVAFII | CD28 transmembrane domain |
| | FWV | |

In certain embodiments, the intracellular signaling domain and/or intracellular costimulatory domain of the CAR may comprise one or more signaling domains selected from B7-1/CD80, B7-2/CD86, B7-H1/PD-L1, B7-H2, B7-H3, B7-H4, B7-H6, B7-H7, BTLA/CD272, CD28, CTLA-4, Gi24NISTA/B7-H5, ICOS/CD278, PD-1, PD-L2/B7-DC, PDCD6, 4-1BB/TNFSF9/CD137, 4-1BB Ligand/TNFSF9, BAFF/BLyS/TNFSF13B, BAFF R/TNFRSF13C, CD27TNFRSF7, CD27 LigandTNFSF7, CD30/TNFRSF8, CD30 Ligand/TNFSF8, CD40/NFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNFβ, OX40/TNFRSF4, OX40 Ligand/TNFSF4, RELT/TNFRSF19L, TACI/TNFRSF13B, TL1A/TNFSF15, TNFα, TNF RII/TNFRSF1B, 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7. NTB-A/SLAMF6, SLAM/CD150, CD2, CD7, CD53, CD82/Kai-1, CD90/Thy1, CD96, CD160, CD200, CD300a/LMIR1. HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), NKG2C, CD3ζ, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, and a functional variant thereof including the human versions of each of these sequences. In some embodiments, the intracellular signaling domain and/or intracellular costimulatory domain comprises one or more signaling domains selected from a CD3ζ domain, an ITAM, a CD28 domain, 4-1BB domain, or a functional variant thereof. Table 7 provides the amino acid sequences of a few exemplary intracellular costimulatory and/or signaling domains. In certain embodiments, as in the case of tisagenlecleucel as described below, the CD3ζ signaling domain of SEQ ID NO:20A may have a mutation, e.g., a glutamine (Q) to lysine (K) mutation, at amino acid position 14 (see SEQ ID NO:21A).

TABLE 7
Exemplary sequences of intracellular costimulatory and/or signaling domains

| SEQ ID NO: | Sequence | Description |
|---|---|---|
| 18A | KRGRKKLLYIFKQPFMRPVQTTQEEDG | 4-1BB costimulatory domain |
| | CSCRFPEEEEGGCEL | |
| 19A | RSKRSRLLHSDYMNMTPRRPGPTRKHY | CD28 costimulatory domain |
| | QPYAPPRDFAAYRS | |
| 20A | RVKFSRSADAPAYQQGQNQLYNELNL | CD3ζ signaling domain |
| | GRREEYDVLDKRRGRDPEMGGKPRRK | |
| | NPQEGLYNELQKDKMAEAYSEIGMKG | |
| | ERRRGKGHDGLYQGLSTATKDTYDAL | |
| | HMQALPPR | |
| 21A | RVKFSRSADAPAYKQGQNQLYNELNL | CD3ζ signaling domain (with Q |
| | GRREEYDVLDKRRGRDPEMGGKPRRK | to K mutation at position 14) |
| | NPQEGLYNELQKDKMAEAYSEIGMKG | |
| | ERRRGKGHDGLYQGLSTATKDTYDAL | |
| | HMQALPPR | |

### F. CD19 CAR

In some embodiments, the CAR is a CD19 CAR, and in these embodiments, the second transgene comprises a nucleotide sequence encoding a CD19 CAR. In some embodiments, the CD19 CAR may comprise a signal peptide, an extracellular binding domain that specifically binds CD19, a hinge domain, a transmembrane domain, an intracellular costimulatory domain, and/or an intracellular signaling domain in tandem.

In some embodiments, the signal peptide of the CD19 CAR comprises a CD8α signal peptide. In some embodiments, the CD8α signal peptide comprises or consists of an amino acid sequence set forth in SEQ ID NO:6A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6A. In some embodiments, the signal peptide comprises an IgK signal peptide. In some embodiments, the IgK signal peptide comprises or consists of an amino acid sequence set forth in SEQ ID NO:7A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7A. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8A.

In some embodiments, the extracellular binding domain of the CD19 CAR is specific to CD19, for example, human CD19. The extracellular binding domain of the CD19 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD19 CAR comprises an scFv derived from the FMC63 monoclonal antibody (FMC63), which comprises the heavy chain variable region (V_{H}) and the light chain variable region (V_{L}) of FMC63 connected by a linker. FMC63 and the derived scFv have been described in Nicholson et al., Mol. Immun. 34(16-17):1157-1165 (1997) and PCT Application Publication No.

WO2018/213337, the entire contents of each of which are incorporated by reference herein. In some embodiments, the amino acid sequences of the entire FMC63-derived scFv (also referred to as FMC63 scFv) and its different portions are provided in Table 8 below. In some embodiments, the CD19-specific scFv comprises or consists of an amino acid sequence set forth in SEQ ID NO: 22A, 23A, or 28A, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 22A, 23A, or 28A. In some embodiments, the CD19-specific scFv may comprise one or more CDRs having amino acid sequences set forth in SEQ ID NOs: 24A, 25A, 26A and 29A, 30A, 31A. In some embodiments, the CD19-specific scFv may comprise a light chain with one or more CDRs having amino acid sequences set forth in SEQ ID NOs: 24A, 25A, 26A. In some embodiments, the CD19-specific scFv may comprise a heavy chain with one or more CDRs having amino acid sequences set forth in SEQ ID NOs: 29A, 30A, 31A. In any of these embodiments, the CD19-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD19 CAR comprises or consists of the one or more CDRs as described herein.

In some embodiments, the linker linking the V_{II} and the V_{L} portions of the scFv is a Whitlow linker having an amino acid sequence set forth in SEQ ID NO:27A. In some embodiments, the Whitlow linker may be replaced by a different linker, for example, a 3×G₄S linker having an amino acid sequence set forth in SEQ ID NO:33A, which gives rise to a different FMC63-derived scFv having an amino acid sequence set forth in SEQ ID NO:32A. In certain of these embodiments, the CD19-specific scFv comprises or consists of an amino acid sequence set forth in SEQ ID NO:32A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:32A.

TABLE 8
Exemplary sequences of anti-CD19 scFv and components

| SEQ ID NO: | Amino Acid Sequence | Description |
|---|---|---|
| 22A | DIQMTQTTSSLSASLGDRVTISCRASQDI | Anti-CD19 FMC63 |
| | SKYLNWYQQKPDGTVKLLIYHTSRLHS | scFv entire sequence, |
| | GVPSRFSGSGSGTDYSLTISNLEQEDIAT | with Whitlow linker |
| | YFCQQGNTLPYTFGGGTKLEITGSTSGS | |
| | GKPGSGEGSTKGEVKLQESGPGLVAPSQ | |
| | SLSVTCTVSGVSLPDYGVSWIRQPPRKG | |
| | LEWLGVIWGSETTYYNSALKSRLTIIKD | |
| | NSKSQVFLKMNSLQTDDTAIYYCAKHY | |
| | YYGGSYAMDYWGQGTSVTVSS | |
| 23A | DIQMTQTTSSLSASLGDRVTISCRASQDI | Anti-CD19 FMC63 |
| | SKYLNWYQQKPDGTVKLLIYHTSRLHS | scFv light chain |
| | GVPSRFSGSGSGTDYSLTISNLEQEDIAT | variable region |
| | YFCQQGNTLPYTFGGGTKLEIT | |
| 24A | QDISKY | Anti-CD19 FMC63 |
| | | scFv light chain CDR1 |
| 25A | HTS | Anti-CD19 FMC63 |
| | | scFv light chain CDR2 |
| 26A | QQGNTLPYT | Anti-CD19 FMC63 |
| | | scFv light chain CDR3 |
| 27A | GSTSGSGKPGSGEGSTKG | Whitlow linker |
| 28A | EVKLQESGPGLVAPSQSLSVTCTVSGVS | Anti-CD19 FMC63 |
| | LPDYGVSWIRQPPRKGLEWLGVIWGSET | scFv heavy chain |
| | TYYNSALKSRLTIIKDNSKSQVFLKMNS | variable region |
| | LQTDDTAIYYCAKHYYYGGSYAMDYW | |
| | GQGTSVTVSS | |
| 29A | GVSLPDYG | Anti-CD19 FMC63 |
| | | scFv heavy chain |
| | | CDR1 |
| 30A | IWGSETT | Anti-CD19 FMC63 |
| | | scFv heavy chain |
| | | CDR2 |
| 31A | AKHYYYGGSYAMDY | Anti-CD19 FMC63 |
| | | scFv heavy chain |
| | | CDR3 |
| 32A | DIQMTQTTSSLSASLGDRVTISCRASQDI | Anti-CD19 FMC63 |
| | SKYLNWYQQKPDGTVKLLIYHTSRLHS | scFv entire sequence, |
| | GVPSRFSGSGSGTDYSLTISNLEQEDIAT | with 3xG4S linker |
| | YFCQQGNTLPYTFGGGTKLEITGGGGSG | |
| | GGGSGGGGSEVKLQESGPGLVAPSQSLS | |
| | VTCTVSGVSLPDYGVSWIRQPPRKGLE | |
| | WLGVIWGSETTYYNSALKSRLTIIKDNS | |
| | KSQVFLKMNSLQTDDTAIYYCAKHYYY | |
| | GGSYAMDYWGQGTSVTVSS | |
| 33A | GGGGSGGGGSGGGGS | 3xG4S linker |

In some embodiments, the extracellular binding domain of the CD19 CAR is derived from an antibody specific to CD19, including, for example, SJ25C1 (Bejcek et al., Cancer Res. 55:2346-2351 (1995)), HD37 (Pezutto et al., J. Immunol. 138(9):2793-2799 (1987)), 4G7 (Meeker et al., Hybridoma 3:305-320 (1984)), B43 (Bejcek (1995)), BLY3 (Bejcek (1995)). B4 (Freedman et al., 70:418-427 (1987)). B4 HB12b (Kansas & Tedder, J. Immunol. 147:4094-4102(1991); Yazawa et al., Proc. Natl. Acad. Sci. USA 102:15178-15183 (2005); Herbst et al., J. Pharmacol. Exp. Ther. 335:213-222 (2010)), BU12 (Callard et al., J. Immunology, 148(10); 2983-2987 (1992)), and CLB-CD19 (De Rie Cell. Immunol. 118:368-381(1989)). In any of these embodiments, the extracellular binding domain of the CD19 CAR can comprise or consist of the V_{H}, the V_{L}, and/or one or more CDRs of any of the antibodies.

In some embodiments, the hinge domain of the CD19 CAR comprises a CD8α hinge domain, for example, a human CD8α hinge domain. In some embodiments, the CD8α hinge domain comprises or consists of an amino acid sequence set forth in SEQ ID NO:9A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9A. In some embodiments, the hinge domain comprises a CD28 hinge domain, for example, a human CD28 hinge domain. In some embodiments, the CD28 hinge domain comprises or consists of an amino acid sequence set forth in SEQ ID NO:10A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:10A. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:12A or SEQ ID NO:13A, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:12A or SEQ ID NO:13A. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:14A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:14A.

In some embodiments, the transmembrane domain of the CD19 CAR comprises a CD8α transmembrane domain, for example, a human CD8α transmembrane domain. In some embodiments, the CD8α transmembrane domain comprises or consists of an amino acid sequence set forth in SEQ ID NO:15A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15A. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:16A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16A.

In some embodiments, the intracellular costimulatory domain of the CD19 CAR comprises a 4-1BB costimulatory domain. 4-1BB, also known as CD137, transmits a potent costimulatory signal to T cells, promoting differentiation and enhancing long-term survival of T lymphocytes. In some embodiments, the 4-1BB costimulatory domain is human. In some embodiments, the 4-1BB costimulatory domain comprises or consists of an amino acid sequence set forth in SEQ ID NO:18A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18A. In some embodiments, the intracellular costimulatory domain comprises a CD28 costimulatory domain. CD28 is another co-stimulatory molecule on T cells. In some embodiments, the CD28 costimulatory domain is human. In some embodiments, the CD28 costimulatory domain comprises or consists of an amino acid sequence set forth in SEQ ID NO:19A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:19A. In some embodiments, the intracellular costimulatory domain of the CD19 CAR comprises a 4-1BB costimulatory domain and a CD28 costimulatory domain as described.

In some embodiments, the intracellular signaling domain of the CD19 CAR comprises a CD3 zeta (ζ) signaling domain. CD3C associates with T cell receptors (TCRs) to produce a signal and contains immunoreceptor tyrosine-based activation motifs (ITAMs). The CD3ζ signaling domain refers to amino acid residues from the cytoplasmic domain of the zeta chain that are sufficient to functionally transmit an initial signal necessary for T cell activation. In some embodiments, the CD3ζ signaling domain is human. In some embodiments, the CD3ζ signaling domain comprises or consists of an amino acid sequence set forth in SEQ ID NO:20A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:20A.

In some embodiments, the second transgene comprises a nucleotide sequence encoding a CD19 CAR, including, for example, a CD19 CAR comprising the CD19-specific scFv having sequences set forth in SEQ ID NO:22A or SEQ ID NO:32A, the CD8α hinge domain of SEQ ID NO:9A, the CD8α transmembrane domain of SEQ ID NO:15A, the 4-1BB costimulatory domain of SEQ ID NO:18A, the CD3ζ signaling domain of SEQ ID NO:20A, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the second transgene comprises a nucleotide sequence encoding a CD19 CAR, including, for example, a CD19 CAR comprising the CD19-specific scFv having sequences set forth in SEQ ID NO:22A or SEQ ID NO:32A, the IgG4 hinge domain of SEQ ID NO:12A or SEQ ID NO:13A, the CD28 transmembrane domain of SEQ ID NO:16A, the 4-1BB costimulatory domain of SEQ ID NO:18A, the CD3ζ signaling domain of SEQ ID NO:20A, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the second transgene comprises a nucleotide sequence encoding a CD19 CAR, including, for example, a CD19 CAR comprising the CD19-specific scFv having sequences set forth in SEQ ID NO:22A or SEQ ID NO:32A, the CD28 hinge domain of SEQ ID NO:10A, the CD28 transmembrane domain of SEQ ID NO:16A, the CD28 costimulatory domain of SEQ ID NO:19A, the CD3ζ signaling domain of SEQ ID NO:20A, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the second transgene comprises a nucleotide sequence encoding a CD19 CAR as set forth in SEQ ID NO:34A or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:34A (see Table 9). The encoded CD19 CAR has a corresponding amino acid sequence set forth in SEQ ID NO:35A or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:35A, with the following components: CD8α signal peptide, FMC63 scFv (V_{L}-Whitlow linker-V_{II}), CD8α hinge domain, CD8α transmembrane domain, 4-1BB costimulatory domain, and CD3ζ signaling domain.

In some embodiments, the second transgene comprises a nucleotide sequence encoding a commercially available embodiment of CD19 CAR. Non-limiting examples of commercially available embodiments of CD19 CARs expressed and/or encoded by T cells include tisagenlecleucel, lisocabtagene maraleucel, axicabtagene ciloleucel, and brexucabtagene autoleucel.

In some embodiments, the second transgene comprises a nucleotide sequence encoding tisagenlecleucel or portions thereof. Tisagenlecleucel comprises a CD19 CAR with the following components: CD8α signal peptide, FMC63 scFv (V_{L}-3×G₄S linker-V_{H}), CD8α hinge domain, CD8α transmembrane domain, 4-1BB costimulatory domain, and CD3ζ signaling domain. The nucleotide and amino acid sequence of the CD19 CAR in tisagenlecleucel are provided in Table 9, with annotations of the sequences provided in Table 10.

In some embodiments, the second transgene comprises a nucleotide sequence encoding lisocabtagene maraleucel or portions thereof. Lisocabtagene maraleucel comprises a CD19 CAR with the following components: GMCSFR-α or CSF2RA signal peptide, FMC63 scFv (V_{L}-Whitlow linker-V_{H}), IgG4 hinge domain, CD28 transmembrane domain, 4-1BB costimulatory domain, and CD3ζ signaling domain. The nucleotide and amino acid sequence of the CD19 CAR in lisocabtagene maraleucel are provided in Table 9, with annotations of the sequences provided in Table 11.

In some embodiments, the second transgene comprises a nucleotide sequence encoding axicabtagene ciloleucel or portions thereof. Axicabtagene ciloleucel comprises a CD19 CAR with the following components: GMCSFR-α or CSF2RA signal peptide, FMC63 scFv (V_{L}-Whitlow linker-V_{H}), CD28 hinge domain, CD28 transmembrane domain, CD28 costimulatory domain, and CD3 signaling domain. The nucleotide and amino acid sequence of the CD19 CAR in axicabtagene ciloleucel are provided in Table 9, with annotations of the sequences provided in Table 12.

In some embodiments, the second transgene comprises a nucleotide sequence encoding brexucabtagene autoleucel or portions thereof. Brexucabtagene autoleucel comprises a CD19 CAR with the following components: GMCSFR-α signal peptide. FMC63 scFv, CD28 hinge domain. CD28 transmembrane domain, CD28 costimulatory domain, and CD3C signaling domain.

In some embodiments, the second transgene comprises a nucleotide sequence encoding a CD19 CAR as set forth in SEQ ID NO: 36A, 38A, or 40A, or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 36A, 38A, or 40A. The encoded CD19 CAR has a corresponding amino acid sequence set forth in SEQ ID NO: 37A, 39A, or 41A, respectively, or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 37A, 39A, or 41A, respectively.

TABLE 9
Exemplary sequences of CD19 CARs

| SEQ ID NO: | Sequence | Description |
|---|---|---|
| 34A | atggccttaccagtgaccgccttgctcctgccgctggccttgctgctccacg | Exemplary |
| | ccgccaggccggacatccagatgacacagactacatcctccctgtctgcct | CD19 CAR |
| | ctctgggagacagagtcaccatcagttgcagggcaagtcaggacattagta | nucleotide |
| | aatatttaaattggtatcagcagaaaccagatggaactgttaaactcctgatct | sequence |
| | accatacatcaagattacactcaggagtcccatcaaggttcagtggcagtg | |
| | ggtctggaacagattattctctcaccattagcaacctggagcaagaagatatt | |
| | gccacttacttttgccaacagggtaatacgcttccgtacacgttcggagggg | |
| | ggaccaagctggagatcacaggctccacctctggatccggcaagcccgg | |
| | atctggcgagggatccaccaagggcgaggtgaaactgcaggagtcagga | |
| | cctggcctggtggcgccctcacagagcctgtccgtcacatgcactgtctca | |
| | ggggtctcattacccgactatggtgtaagctggattcgccagcctccacgaa | |
| | agggtctggagtggctgggagtaatatggggtagtgaaaccacatactata | |
| | attcagctctcaaatccagactgaccatcatcaaggacaactccaagagcc | |
| | aagttttcttaaaaatgaacagtctgcaaactgatgacacagccatttactact | |
| | gtgccaaacattattactacggtggtagctatgctatggactactggggcca | |
| | aggaacctcagtcaccgtctcctcaaccacgacgccagcgccgcgacca | |
| | ccaacaccggcgcccaccatcgcgtcgcagcccctgtccctgcgcccag | |
| | aggcgtgccggccagcggcggggggcgcagtgcacacgagggggctg | |
| | gacttcgcctgtgatatctacatctgggcgcccttggccgggacttgtgggg | |
| | tccttctcctgtcactggttatcaccctttactgcaaacggggcagaaagaaa | |
| | ctcctgtatatattcaaacaaccatttatgagaccagtacaaactactcaaga | |
| | ggaagatggctgtagctgccgatttccagaagaagaagaaggaggatgtg | |
| | aactgagagtgaagttcagcaggagcgcagacgcccccgcgtaccagca | |
| | gggccagaaccagctctataacgagctcaatctaggacgaagagaggagt | |
| | acgatgttttggacaagagacgtggccgggaccctgagatggggggaaa | |
| | gccgagaaggaagaaccctcaggaaggcctgtacaatgaactgcagaaa | |
| | gataagatggcggaggcctacagtgagattgggatgaaaggcgagcgcc | |
| | ggaggggcaaggggcacgatggcctttaccagggtctcagtacagccac | |
| | caaggacacctacgacgcccttcacatgcaggccctgccccctcgc | |
| 35A | MALPVTALLLPLALLLHAARPDIQMTQTTSSLS | Exemplary |
| | ASLGDRVTISCRASQDISKYLNWYQQKPDGTVK | CD19 CAR |
| | LLIYHTSRLHSGVPSRFSGSGSGTDYSLTISNLEQ | amino acid |
| | EDIATYFCQQGNTLPYTFGGGTKLEITGSTSGSG | sequence |
| | KPGSGEGSTKGEVKLQESGPGLVAPSQSLSVTC | |
| | TVSGVSLPDYGVSWIRQPPRKGLEWLGVIWGSE | |
| | TTYYNSALKSRLTIIKDNSKSQVFLKMNSLQTD | |
| | DTAIYYCAKHYYYGGSYAMDYWGQGTSVTVS | |
| | STTTPAPRPPTPAPTIASQPLSLRPEACRPAAGGA | |
| | VHTRGLDFACDIYIWAPLAGTCGVLLLSLVITLY | |
| | CKRGRKKLLYIFKQPFMRPVQTTQEEDGCSCRF | |
| | PEEEEGGCELRVKFSRSADAPAYQQGQNQLYN | |
| | ELNLGRREEYDVLDKRRGRDPEMGGKPRRKNP | |
| | QEGLYNELQKDKMAEAYSEIGMKGERRRGKG | |
| | HDGLYQGLSTATKDTYDALHMQALPPR | |
| 36A | atggccttaccagtgaccgccttgctcctgccgctggccttgctgctccacg | Tisagenlecleucel |
| | ccgccaggccggacatccagatgacacagactacatcctccctgtctgcct | CD19 CAR |
| | ctctgggagacagagtcaccatcagttgcagggcaagtcaggacattagta | nucleotide |
| | aatatttaaattggtatcagcagaaaccagatggaactgttaaactcctgatct | sequence |
| | accatacatcaagattacactcaggagtcccatcaaggttcagtggcagtg | |
| | ggtctggaacagattattctctcaccattagcaacctggagcaagaagatatt | |
| | gccacttacttttgccaacagggtaatacgcttccgtacacgttcggagggg | |
| | ggaccaagctggagatcacaggtggcggtggctcgggcggtggtgggtc | |
| | gggtggcggcggatctgaggtgaaactgcaggagtcaggacctggcctg | |
| | gtggcgccctcacagagccigtccgtcacatgcactgtctcaggggtctcat | |
| | tacccgactatggtgtaagctggattcgccagcctccacgaaagggtctgg | |
| | agtggctgggagtaatatggggtagtgaaaccacatactataattcagctct | |
| | caaatccagactgaccatcatcaaggacaactccaagagccaagttttctta | |
| | aaaatgaacagtctgcaaactgatgacacagccatttactactgtgccaaac | |
| | attattactacggtggtagctatgctatggactactggggccaaggaacctc | |
| | agtcaccgtctcctcaaccacgacgccagcgccgcgaccaccaacaccg | |
| | gcgcccaccatcgcgtcgcagcccctgtccctgcgcccagaggcgtgcc | |
| | ggccagcggcggggggcgcagtgcacacgagggggctggacttcgcct | |
| | gtgatatctacatctgggcgcccttggccgggacttgtggggtccttctcctg | |
| | tcactggttatcaccctttactgcaaacggggcagaaagaaactcctgtatat | |
| | attcaaacaaccatttatgagaccagtacaaactactcaagaggaagatgg | |
| | ctgtagctgccgatttccagaagaagaagaaggaggatgtgaactgagag | |
| | tgaagttcagcaggagcgcagacgcccccgcgtacaagcagggccaga | |
| | accagctctataacgagctcaatctaggacgaagagaggagtacgatgtttt | |
| | ggacaagagacgtggccgggaccctgagatggggggaaagccgagaa | |
| | ggaagaaccctcaggaaggcctgtacaatgaactgcagaaagataagatg | |
| | gcggaggcctacagtgagattgggatgaaaggcgagcgccggaggggc | |
| | aaggggcacgatggcctttaccagggtctcagtacagccaccaaggacac | |
| | ctacgacgcccttcacatgcaggccctgccccctcgc | |
| 37A | MALPVTALLLPLALLLHAARPDIQMTQTTSSLS | Tisagenlecleucel |
| | ASLGDRVTISCRASQDISKYLNWYQQKPDGTVK | CD19 CAR |
| | LLIYHTSRLHSGVPSRFSGSGSGTDYSLTISNLEQ | amino acid |
| | EDIATYFCQQGNTLPYTFGGGTKLEITGGGGSG | sequence |
| | GGGSGGGGSEVKLQESGPGLVAPSQSLSVTCTV | |
| | SGVSLPDYGVSWIRQPPRKGLEWLGVIWGSETT | |
| | YYNSALKSRLTIIKDNSKSQVFLKMNSLQTDDT | |
| | AIYYCAKHYYYGGSYAMDYWGQGTSVTVSST | |
| | TTPAPRPPTPAPTIASQPLSLRPEACRPAAGGAV | |
| | HTRGLDFACDIYIWAPLAGTCGVLLLSLVITLYC | |
| | KRGRKKLLYIFKQPFMRPVQTTQEEDGCSCRFP | |
| | EEEEGGCELRVKFSRSADAPAYKQGQNQLYNE | |
| | LNLGRREEYDVLDKRRGRDPEMGGKPRRKNPQ | |
| | EGLYNELQKDKMAEAYSEIGMKGERRRGKGH | |
| | DGLYQGLSTATKDTYDALHMQALPPR | |
| 38A | atgctgctgctggtgaccagcctgctgctgtgcgagctgccccaccccgcc | Lisocabtagene |
| | tttctgctgatccccgacatccagatgacccagaccacctccagcctgagc | maraleucel |
| | gccagcctgggcgaccgggtgaccatcagctgccgggccagccaggac | CD19 CAR |
| | atcagcaagtacctgaactggtatcagcagaagcccgacggcaccgtcaa | nucleotide |
| | gctgctgatctaccacaccagccggctgcacagcggcgtgcccagccggt | sequence |
| | ttagcggcagcggctccggcaccgactacagcctgaccatctccaacctg | |
| | gaacaggaagatatcgccacctacttttgccagcagggcaacacactgcc | |
| | ctacacctttggcggcggaacaaagctggaaatcaccggcagcacctccg | |
| | gcagcggcaagcctggcagcggcgagggcagcaccaagggcgaggtg | |
| | aagctgcaggaaagcggccctggcctggtggcccccagccagagcctga | |
| | gcgtgacctgcaccgtgagcggcgtgagcctgcccgactacggcgtgag | |
| | ctggatccggcagccccccaggaagggcctggaatggctgggcgtgatc | |
| | tggggcagcgagaccacctactacaacagcgccctgaagagccggctga | |
| | ccatcatcaaggacaacagcaagagccaggtgttcctgaagatgaacagc | |
| | ctgcagaccgacgacaccgccatctactactgcgccaagcactactactac | |
| | ggcggcagctacgccatggactactggggccagggcaccagcgtgacc | |
| | gtgagcagcgaatctaagtacggaccgccctgccccccttgccctatgttct | |
| | gggtgctggtggtggtcggaggcgtgctggcctgctacagcctgctggtc | |
| | accgtggccttcatcatcttttgggtgaaacggggcagaaagaaactcctgt | |
| | atatattcaaacaaccatttatgagaccagtacaaactactcaagaggaaga | |
| | tggctgtagctgccgatttccagaagaagaagaaggaggatgtgaactgc | |
| | gggtgaagttcagcagaagcgccgacgcccctgcctaccagcagggcca | |
| | gaatcagctgtacaacgagctgaacctgggcagaagggaagagtacgac | |
| | gtcctggataagcggagaggccgggaccctgagatgggcggcaagcctc | |
| | ggcggaagaacccccaggaaggcctgtataacgaactgcagaaagacaa | |
| | gatggccgaggcctacagcgagatcggcatgaagggcgagcggaggcg | |
| | gggcaagggccacgacggcctgtatcagggcctgtccaccgccaccaag | |
| | gatacctacgacgccctgcacatgcaggccctgcccccaagg | |
| 39A | MLLLVTSLLLCELPHPAFLLIPDIQMTQTTSSLSA | Lisocabtagene |
| | SLGDRVTISCRASQDISKYLNWYQQKPDGTVKL | maraleucel |
| | LIYHTSRLHSGVPSRFSGSGSGTDYSLTISNLEQE | CD19 CAR |
| | DIATYFCQQGNTLPYTFGGGTKLEITGSTSGSGK | amino acid |
| | PGSGEGSTKGEVKLQESGPGLVAPSQSLSVTCT | sequence |
| | VSGVSLPDYGVSWIRQPPRKGLEWLGVIWGSET | |
| | TYYNSALKSRLTIIKDNSKSQVFLKMNSLQTDD | |
| | TAIYYCAKHYYYGGSYAMDYWGQGTSVTVSS | |
| | ESKYGPPCPPCPMFWVLVVVGGVLACYSLLVT | |
| | VAFIIFWVKRGRKKLLYIFKQPFMRPVQTTQEE | |
| | DGCSCRFPEEEEGGCELRVKFSRSADAPAYQQG | |
| | QNQLYNELNLGRREEYDVLDKRRGRDPEMGG | |
| | KPRRKNPQEGLYNELQKDKMAEAYSEIGMKGE | |
| | RRRGKGHDGLYQGLSTATKDTYDALHMQALPP | |
| | R | |
| 40A | atgcttctcctggtgacaagccttctgctctgtgagttaccacacccagcattc | Axicabtagene |
| | ctcctgatcccagacatccagatgacacagactacatcctccctgtctgcct | ciloleucel CD19 |
| | ctctgggagacagagtcaccatcagttgcagggcaagtcaggacattagta | CAR nucleotide |
| | aatatttaaattggtatcagcagaaaccagatggaactgttaaactcctgatct | sequence |
| | accatacatcaagattacactcaggagtcccatcaaggttcagtggcagtg | |
| | ggtctggaacagattattctctcaccattagcaacctggagcaagaagatatt | |
| | gccacttacttttgccaacagggtaatacgcttccgtacacgttcggagggg | |
| | ggactaagttggaaataacaggctccacctctggatccggcaagcccgga | |
| | tctggcgagggatccaccaagggcgaggtgaaactgcaggagtcaggac | |
| | ctggcctggtggcgccctcacagagcctgtccgtcacatgcactgtctcag | |
| | gggtctcattacccgactatggtgtaagctggattcgccagcctccacgaaa | |
| | gggtctggagtggctgggagtaatatggggtagtgaaaccacatactataa | |
| | ttcagctctcaaatccagactgaccatcatcaaggacaactccaagagcca | |
| | agttttcttaaaaatgaacagtctgcaaactgatgacacagccatttactactg | |
| | tgccaaacattattactacggtggtagctatgctatggactactggggtcaag | |
| | gaacctcagtcaccgtctcctcagcggccgcaattgaagttatgtatcctcct | |
| | ccttacctagacaatgagaagagcaatggaaccattatccatgtgaaaggg | |
| | aaacacctttgtccaagtcccctatttcccggaccttctaagcccttttgggtg | |
| | ctggtggtggttgggggagtcctggcttgctatagcttgctagtaacagtgg | |
| | cctttattattttctgggtgaggagtaagaggagcaggctcctgcacagtga | |
| | ctacatgaacatgactccccgccgccccgggcccacccgcaagcattacc | |
| | agccctatgccccaccacgcgacttcgcagcctatcgctccagagtgaagt | |
| | tcagcaggagcgcagacgcccccgcgtaccagcagggccagaaccagc | |
| | tctataacgagctcaatctaggacgaagagaggagtacgatgttttggacaa | |
| | gagacgtggccgggaccctgagatggggggaaagccgagaaggaaga | |
| | accctcaggaaggcctgtacaatgaactgcagaaagataagatggcggag | |
| | gcctacagtgagattgggatgaaaggcgagcgccggaggggcaagggg | |
| | cacgatggcctttaccagggtctcagtacagccaccaaggacacctacga | |
| | cgcccttcacatgcaggccctgccccctcgc | |
| 41A | MLLLVTSLLLCELPHPAFLLIPDIQMTQTTSSLSA | Axicabtagene |
| | SLGDRVTISCRASQDISKYLNWYQQKPDGTVKL | ciloleucel CD19 |
| | LIYHTSRLHSGVPSRFSGSGSGTDYSLTISNLEQE | CAR amino acid |
| | DIATYFCQQGNTLPYTFGGGTKLEITGSTSGSGK | sequence |
| | PGSGEGSTKGEVKLQESGPGLVAPSQSLSVTCT | |
| | VSGVSLPDYGVSWIRQPPRKGLEWLGVIWGSET | |
| | TYYNSALKSRLTIIKDNSKSQVFLKMNSLQTDD | |
| | TAIYYCAKHYYYGGSYAMDYWGQGTSVTVSS | |
| | AAAIEVMYPPPYLDNEKSNGTIIHVKGKHLCPSP | |
| | LFPGPSKPFWVLVVVGGVLACYSLLVTVAFIIF | |
| | WVRSKRSRLLHSDYMNMTPRRPGPTRKHYQPY | |
| | APPRDFAAYRSRVKFSRSADAPAYQQGQNQLY | |
| | NELNLGRREEYDVLDKRRGRDPEMGGKPRRKN | |
| | PQEGLYNELQKDKMAEAYSEIGMKGERRRGKG | |
| | HDGLYQGLSTATKDTYDALHMQALPPR | |

TABLE 10
Annotation of tisagenlecleucel CD19 CAR sequences

| | Nucleotide | Amino Acid |
|---|---|---|
| | Sequence | Sequence |
| Feature | Position | Position |
| CD8α signal peptide | 1-63 | 1-21 |
| FMC63 scFv | 64-789 | 22-263 |
| (V_{L}-3 × G₄S linker-V_{H}) | | |
| CD8α hinge domain | 790-924 | 264-308 |
| CD8α transmembrane domain | 925-996 | 309-332 |
| 4-1BB costimulatory domain | 997-1122 | 333-374 |
| CD3ζ signaling domain | 1123-1458 | 375-486 |

TABLE 11
Annotation of lisocabtagene maraleucel CD19 CAR sequences

| | Nucleotide | Amino Acid |
|---|---|---|
| | Sequence | Sequence |
| Feature | Position | Position |
| GMCSFR-α signal peptide | 1-66 | 1-22 |
| FMC63 scFv | 67-801 | 23-267 |
| (V_{L}-Whitlow linker-V_{H}) | | |
| IgG4 hinge domain | 802-837 | 268-279 |
| CD28 transmembrane domain | 838-921 | 280-307 |
| 4-1BB costimulatory domain | 922-1047 | 308-349 |
| CD3ζ signaling domain | 1048-1383 | 350-461 |

TABLE 12
Annotation of axicabtagene ciloleucel CD19 CAR sequences

| | Nucleotide | Amino Acid |
|---|---|---|
| | Sequence | Sequence |
| Feature | Position | Position |
| CSF2RA signal peptide | 1-66 | 1-22 |
| FMC63 scFv | 67-801 | 23-267 |
| (V_{L}-Whitlow linker-V_{H}) | | |
| CD28 hinge domain | 802-927 | 268-309 |
| CD28 transmembrane domain | 928-1008 | 310-336 |
| CD28 costimulatory domain | 1009-1131 | 337-377 |
| CD3ζ signaling domain | 1132-1467 | 378-489 |

In some embodiments, the second transgene comprises a nucleotide sequence encoding CD19 CAR as set forth in SEQ ID NO: 31A, 33A, or 35A, or at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 31A, 33A, or 35A. The encoded CD19 CAR has a corresponding amino acid sequence set forth in SEQ ID NO: 32A, 34A, or 36A, respectively, is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 32A, 34A, or 36A, respectively.

### G. CD20 CAR

In some embodiments, the CAR is a CD20 CAR, and in these embodiments, the second transgene comprises a nucleotide sequence encoding a CD20 CAR CD20 is an antigen found on the surface of B cells as early at the pro-B phase and progressively at increasing levels until B cell maturity, as well as on the cells of most B-cell neoplasms. CD20 positive cells are also sometimes found in cases of Hodgkin's disease, myeloma, and thymoma. In some embodiments, the CD20 CAR may comprise a signal peptide, an extracellular binding domain that specifically binds CD20, a hinge domain, a transmembrane domain, an intracellular costimulatory domain, and/or an intracellular signaling domain in tandem.

In some embodiments, the signal peptide of the CD20 CAR comprises a CD8α signal peptide. In some embodiments, the CD8α signal peptide comprises or consists of an amino acid sequence set forth in SEQ ID NO:6A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6A. In some embodiments, the signal peptide comprises an IgK signal peptide. In some embodiments, the IgK signal peptide comprises or consists of an amino acid sequence set forth in SEQ ID NO:7A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7A. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8A.

In some embodiments, the extracellular binding domain of the CD20 CAR is specific to CD20, for example, human CD20. The extracellular binding domain of the CD20 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD20 CAR is derived from an antibody specific to CD20, including, for example, Leu16, IFS, 1.5.3, rituximab, obinutuzumab, ibritumomab, ofatumumab, tositumumab, odronextamab, veltuzumab, ublituximab, and ocrelizumab. In any of these embodiments, the extracellular binding domain of the CD20 CAR can comprise or consist of the V_{H}, the V_{L}, and/or one or more CDRs of any of the antibodies.

In some embodiments, the extracellular binding domain of the CD20 CAR comprises an scFv derived from the Leu16 monoclonal antibody, which comprises the heavy chain variable region (V_{H}) and the light chain variable region (V_{L}) of Leu16 connected by a linker. See Wu et al., Protein Engineering. 14(12):1025-1033 (2001). In some embodiments, the linker is a 3×G₄S linker. In other embodiments, the linker is a Whitlow linker as described herein. In some embodiments, the amino acid sequences of different portions of the entire Leu16-derived scFv (also referred to as Leu16 scFv) and its different portions are provided in Table 13 below. In some embodiments, the CD20-specific scFv comprises or consists of an amino acid sequence set forth in SEQ ID NO: 42A, 43A, or 47A, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 42A, 43A, or 47A. In some embodiments, the CD20-specific scFv may comprise one or more CDRs having amino acid sequences set forth in SEQ ID NOs: 44A, 45A, 46A, 48A, and 49A. In some embodiments, the CD20-specific scFv may comprise a light chain with one or more CDRs having amino acid sequences set forth in SEQ ID NOs: 44A, 45A, 46A. In some embodiments, the CD20-specific scFv may comprise a heavy chain with one or more CDRs having amino acid sequences set forth in SEQ ID NOs: 48A, 49A. In any of these embodiments, the CD20-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD20 CAR comprises or consists of the one or more CDRs as described herein.

TABLE 13
Exemplary sequences of anti-CD20 scFv and components

| SEQ ID NO: | Amino Acid Sequence | Description |
|---|---|---|
| 42A | DIVLTQSPAILSASPGEKVTMTCRASSSVNYM | Anti-CD20 Leu16 scFv |
| | DWYQKKPGSSPKPWIYATSNLASGVPARFSGS | entire sequence, with |
| | GSGTSYSLTISRVEAEDAATYYCQQWSFNPPT | Whitlow linker |
| | FGGGTKLEIKGSTSGSGKPGSGEGSTKGEVQL | |
| | QQSGAELVKPGASVKMSCKASGYTFTSYNMH | |
| | WVKQTPGQGLEWIGAIYPGNGDTSYNQKFKG | |
| | KATLTADKSSSTAYMQLSSLTSEDSADYYCAR | |
| | SNYYGSSYWFFDVWGAGTTVTVSS | |
| 43A | DIVLTQSPAILSASPGEKVTMTCRASSSVNYM | Anti-CD20 Leu16 scFv |
| | DWYQKKPGSSPKPWIYATSNLASGVPARFSGS | light chain variable |
| | GSGTSYSLTISRVEAEDAATYYCQQWSFNPPT | region |
| | FGGGTKLEIK | |
| 44A | RASSSVNYMD | Anti-CD20 Leu16 scFv |
| | | light chain CDR1 |
| 45A | ATSNLAS | Anti-CD20 Leu16 scFv |
| | | light chain CDR2 |
| 46A | QQWSFNPPT | Anti-CD20 Leu16 scFv |
| | | light chain CDR3 |
| 47A | EVQLQQSGAELVKPGASVKMSCKASGYTFTS | Anti-CD20 Leu16 scFv |
| | YNMHWVKQTPGQGLEWIGAIYPGNGDTSYN | heavy chain |
| | QKFKGKATLTADKSSSTAYMQLSSLTSEDSAD | |
| | YYCARSNYYGSSYWFFDVWGAGTTVTVSS | |
| 48A | SYNMH | Anti-CD20 Leu16 scFv |
| | | heavy chain CDR1 |
| 49A | AIYPGNGDTSYNQKFKG | Anti-CD20 Leu16 scFv |
| | | heavy chain CDR2 |

In some embodiments, the hinge domain of the CD20 CAR comprises a CD8α hinge domain, for example, a human CD8α hinge domain. In some embodiments, the CD8α hinge domain comprises or consists of an amino acid sequence set forth in SEQ ID NO:9A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9A. In some embodiments, the hinge domain comprises a CD28 hinge domain, for example, a human CD28 hinge domain. In some embodiments, the CD28 hinge domain comprises or consists of an amino acid sequence set forth in SEQ ID NO:10A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:10A. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:12A or SEQ ID NO:13A, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:12A or SEQ ID NO:13A. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:14A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:14A.

In some embodiments, the transmembrane domain of the CD20 CAR comprises a CD8α transmembrane domain, for example, a human CD8α transmembrane domain. In some embodiments, the CD8α transmembrane domain comprises or consists of an amino acid sequence set forth in SEQ ID NO:15A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15A. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:16A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16A.

In some embodiments, the intracellular costimulatory domain of the CD20 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:18A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18A. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:19A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:19A.

In some embodiments, the intracellular signaling domain of the CD20 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:20A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:20A.

In some embodiments, the second transgene comprises a nucleotide sequence encoding a CD20 CAR, including, for example, a CD20 CAR comprising the CD20-specific scFv having sequences set forth in SEQ ID NO:42A, the CD8α hinge domain of SEQ ID NO:9A, the CD8α transmembrane domain of SEQ ID NO:15A, the 4-1BB costimulatory domain of SEQ ID NO:18A, the CD3ζ signaling domain of SEQ ID NO:20A, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the second transgene comprises a nucleotide sequence encoding a CD20 CAR, including, for example, a CD20 CAR comprising the CD20-specific scFv having sequences set forth in SEQ ID NO:42A, the CD28 hinge domain of SEQ ID NO:10A, the CD8α transmembrane domain of SEQ ID NO:15A, the 4-1BB costimulatory domain of SEQ ID NO:18A, the CD3ζ signaling domain of SEQ ID NO:20A, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the second transgene comprises a nucleotide sequence encoding a CD20 CAR, including, for example, a CD20 CAR comprising the CD20-specific scFv having sequences set forth in SEQ ID NO:42A, the IgG4 hinge domain of SEQ ID NO:12A or SEQ ID NO:13A, the CD8α transmembrane domain of SEQ ID NO:15A, the 4-1BB costimulatory domain of SEQ ID NO:18A, the CD3ζ signaling domain of SEQ ID NO:20A, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the second transgene comprises a nucleotide sequence encoding a CD20 CAR, including, for example, a CD20 CAR comprising the CD20-specific scFv having sequences set forth in SEQ ID NO:42A, the CD8α hinge domain of SEQ ID NO:9A, the CD28 transmembrane domain of SEQ ID NO:16A, the 4-IBB costimulatory domain of SEQ ID NO:18A, the CD3ζ signaling domain of SEQ ID NO:20A, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the second transgene comprises a nucleotide sequence encoding a CD20 CAR, including, for example, a CD20 CAR comprising the CD20-specific scFv having sequences set forth in SEQ ID NO:42A, the CD28 hinge domain of SEQ ID NO:10A, the CD28 transmembrane domain of SEQ ID NO:16A, the 4-IBB costimulatory domain of SEQ ID NO:18A, the CD3ζ signaling domain of SEQ ID NO:20A, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the second transgene comprises a nucleotide sequence encoding a CD20 CAR, including, for example, a CD20 CAR comprising the CD20-specific scFv having sequences set forth in SEQ ID NO:42A, the IgG4 hinge domain of SEQ ID NO:12A or SEQ ID NO:13A, the CD28 transmembrane domain of SEQ ID NO:16A, the 4-1BB costimulatory domain of SEQ ID NO:18A, the CD3ζ signaling domain of SEQ ID NO:20A, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

### H. CD22 CAR

In some embodiments, the CAR is a CD22 CAR, and in these embodiments, the second transgene comprises a nucleotide sequence encoding a CD22 CAR. CD22, which is a transmembrane protein found mostly on the surface of mature B cells that functions as an inhibitory receptor for B cell receptor (BCR) signaling. CD22 is expressed in 60-70% of B cell lymphomas and leukemias (e.g., B-chronic lymphocytic leukemia, hairy cell leukemia, acute lymphocytic leukemia (ALL), and Burkitt's lymphoma) and is not present on the cell surface in early stages of B cell development or on stem cells. In some embodiments, the CD22 CAR may comprise a signal peptide, an extracellular binding domain that specifically binds CD22, a hinge domain, a transmembrane domain, an intracellular costimulatory domain, and/or an intracellular signaling domain in tandem.

In some embodiments, the signal peptide of the CD22 CAR comprises a CD8α signal peptide. In some embodiments, the CD8α signal peptide comprises or consists of an amino acid sequence set forth in SEQ ID NO:6A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6A. In some embodiments, the signal peptide comprises an IgK signal peptide. In some embodiments, the IgK signal peptide comprises or consists of an amino acid sequence set forth in SEQ ID NO:7A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7A. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8A.

In some embodiments, the extracellular binding domain of the CD22 CAR is specific to CD22, for example, human CD22. The extracellular binding domain of the CD22 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD22 CAR is derived from an antibody specific to CD22, including, for example, SM03, inotuzumab, epratuzumab, moxetumomab, and pinatuzumab. In any of these embodiments, the extracellular binding domain of the CD22 CAR can comprise or consist of the V_{H}, the V_{L}, and/or one or more CDRs of any of the antibodies.

In some embodiments, the extracellular binding domain of the CD22 CAR comprises an scFv derived from the m971 monoclonal antibody (m971), which comprises the heavy chain variable region (V_{H}) and the light chain variable region (V_{L}) of m971 connected by a linker. In some embodiments, the linker is a 3×G₄S linker. In other embodiments, the Whitlow linker may be used instead. In some embodiments, the amino acid sequences of the entire m971-derived scFv (also referred to as m971 scFv) and its different portions are provided in Table 14 below. In some embodiments, the CD22-specific scFv comprises or consists of an amino acid sequence set forth in SEQ ID NO: 50A, 51A, or 55A, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 50A, 51A, or 55A. In some embodiments, the CD22-specific scFv may comprise one or more CDRs having amino acid sequences set forth in SEQ ID NOs: 52A, 53A, 54A and 56A, 57A, 58A. In some embodiments, the CD22-specific scFv may comprise a heavy chain with one or more CDRs having amino acid sequences set forth in SEQ ID NOs: 52A, 53A, 54A. In some embodiments, the CD22-specific scFv may comprise a light chain with one or more CDRs having amino acid sequences set forth in SEQ ID NOs: 56A, 57A, 58A. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

In some embodiments, the extracellular binding domain of the CD22 CAR comprises an scFv derived from m971-L7, which is an affinity matured variant of m971 with significantly improved CD22 binding affinity compared to the parental antibody m971 (improved from about 2 nM to less than 50 pM). In some embodiments, the scFv derived from m971-L7 comprises the V_{H} and the V_{L} of m971-L7 connected by a 3×G₄S linker. In other embodiments, the Whitlow linker may be used instead. In some embodiments, the amino acid sequences of the entire m971-L7-derived scFv (also referred to as m971-L7 scFv) and its different portions are provided in Table 14 below. In some embodiments, the CD22-specific scFv comprises or consists of an amino acid sequence set forth in SEQ ID NO: 59A, 60A, or 64A, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 59A, 60A, or 64A. In some embodiments, the CD22-specific scFv may comprise one or more CDRs having amino acid sequences set forth in SEQ ID NOs: 61A, 62A, 63A and 65A, 66A, 67A. In some embodiments, the CD22-specific scFv may comprise a heavy chain with one or more CDRs having amino acid sequences set forth in SEQ ID NOs: 61A, 62A, 63A. In some embodiments, the CD22-specific scFv may comprise a light chain with one or more CDRs having amino acid sequences set forth in SEQ ID NOs: 65A, 66A, 67A. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

TABLE 14
Exemplary sequences of anti-CD22 scFv and components

| SEQ ID NO: | Amino Acid Sequence | Description |
|---|---|---|
| 50A | QVQLQQSGPGLVKPSQTLSLTCAISGDSVSSN | Anti-CD22 m971 |
| | SAAWNWIRQSPSRGLEWLGRTYYRSKWYND | scFv entire sequence, |
| | YAVSVKSRITINPDTSKNQFSLQLNSVTPEDT | with 3xG₄S linker |
| | AVYYCAREVTGDLEDAFDIWGQGTMVTVSS | |
| | GGGGSGGGGSGGGGSDIQMTQSPSSLSASVG | |
| | DRVTITCRASQTIWSYLNWYQQRPGKAPNLL | |
| | IYAASSLQSGVPSRFSGRGSGTDFTLTISSLQA | |
| | EDFATYYCQQSYSIPQTFGQGTKLEIK | |
| 51A | QVQLQQSGPGLVKPSQTLSLTCAISGDSVSSN | Anti-CD22 m971 |
| | SAAWNWIRQSPSRGLEWLGRTYYRSKWYND | scFv heavy chain |
| | YAVSVKSRITINPDTSKNQFSLQLNSVTPEDT | variable region |
| | AVYYCAREVTGDLEDAFDIWGQGTMVTVSS | |
| 52A | GDSVSSNSAA | Anti-CD22 m971 |
| | | scFv heavy chain |
| | | CDR1 |
| 53A | TYYRSKWYN | Anti-CD22 m971 |
| | | scFv heavy chain |
| | | CDR2 |
| 54A | AREVTGDLEDAFDI | Anti-CD22 m971 |
| | | scFv heavy chain |
| | | CDR3 |
| 55A | DIQMTQSPSSLSASVGDRVTITCRASQTIWSY | Anti-CD22 m971 |
| | LNWYQQRPGKAPNLLIYAASSLQSGVPSRFS | scFv light chain |
| | GRGSGTDFTLTISSLQAEDFATYYCQQSYSIP | |
| | QTFGQGTKLEIK | |
| 56A | QTIWSY | Anti-CD22 m971 |
| | | scFv light chain |
| | | CDR1 |
| 57A | AAS | Anti-CD22 m971 |
| | | scFv light chain |
| | | CDR2 |
| 58A | QQSYSIPQT | Anti-CD22 m971 |
| | | scFv light chain |
| | | CDR3 |
| 59A | QVQLQQSGPGMVKPSQTLSLTCAISGDSVSSN | Anti-CD22 m971-L7 |
| | SVAWNWIRQSPSRGLEWLGRTYYRSTWYND | scFv entire sequence, |
| | YAVSMKSRITINPDTNKNQFSLQLNSVTPEDT | with 3xG4S linker |
| | AVYYCAREVTGDLEDAFDIWGQGTMVTVSS | |
| | GGGGSGGGGSGGGGSDIQMIQSPSSLSASVG | |
| | DRVTITCRASQTIWSYLNWYRQRPGEAPNLLI | |
| | YAASSLQSGVPSRFSGRGSGTDFTLTISSLQAE | |
| | DFATYYCQQSYSIPQTFGQGTKLEIK | |
| 60A | QVQLQQSGPGMVKPSQTLSLTCAISGDSVSSN | Anti-CD22 m971-L7 |
| | SVAWNWIRQSPSRGLEWLGRTYYRSTWYND | scFv heavy chain |
| | YAVSMKSRITINPDTNKNQFSLQLNSVTPEDT | variable region |
| | AVYYCAREVTGDLEDAFDIWGQGTMVTVSS | |
| 61A | GDSVSSNSVA | Anti-CD22 m971-L7 |
| | | scFv heavy chain |
| | | CDR1 |
| 62A | TYYRSTWYN | Anti-CD22 m971-L7 |
| | | scFv heavy chain |
| | | CDR2 |
| 63A | AREVTGDLEDAFDI | Anti-CD22 m971-L7 |
| | | scFv heavy chain |
| | | CDR3 |
| 64A | DIQMIQSPSSLSASVGDRVTITCRASQTIWSYL | Anti-CD22 m971-L7 |
| | NWYRQRPGEAPNLLIYAASSLQSGVPSRFSGR | scFv light chain |
| | GSGTDFTLTISSLQAEDFATYYCQQSYSIPQTF | variable region |
| | GQGTKLEIK | |
| 65A | QTIWSY | Anti-CD22 m971-L7 |
| | | scFv light chain |
| | | CDR1 |
| 66A | AAS | Anti-CD22 m971-L7 |
| | | scFv light chain |
| | | CDR2 |
| 67A | QQSYSIPQT | Anti-CD22 m971-L7 |
| | | scFv light chain |
| | | CDR3 |

In some embodiments, the extracellular binding domain of the CD22 CAR comprises immunotoxins HA22 or BL22. Immunotoxins BL22 and HA22 are therapeutic agents that comprise an scFv specific for CD22 fused to a bacterial toxin, and thus can bind to the surface of the cancer cells that express CD22 and kill the cancer cells. BL22 comprises a dsFv of an anti-CD22 antibody, RFB4, fused to a 38-kDa truncated form of Pseudomonas exotoxin A (Bang et al., Clin. Cancer Res., 11:1545-50 (2005)). HA22 (CAT8015, moxetumomab pasudotox) is a mutated, higher affinity version of BL22 (Ho et al., J. Biol. Chem., 280(1); 607-17 (2005)). Suitable sequences of antigen binding domains of HA22 and BL22 specific to CD22 are disclosed in, for example, U.S. Pat. Nos. 7,541,034; 7,355,012; and 7,982,011, which are hereby incorporated by reference in their entirety.

In some embodiments, the hinge domain of the CD22 CAR comprises a CD8α hinge domain, for example, a human CD8α hinge domain. In some embodiments, the CD8α hinge domain comprises or consists of an amino acid sequence set forth in SEQ ID NO:9A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9A. In some embodiments, the hinge domain comprises a CD28 hinge domain, for example, a human CD28 hinge domain. In some embodiments, the CD28 hinge domain comprises or consists of an amino acid sequence set forth in SEQ ID NO:10A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:10A. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:12A or SEQ ID NO:13A, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:12A or SEQ ID NO:13A. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:14A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:14A.

In some embodiments, the transmembrane domain of the CD22 CAR comprises a CD8α transmembrane domain, for example, a human CD8α transmembrane domain. In some embodiments, the CD8α transmembrane domain comprises or consists of an amino acid sequence set forth in SEQ ID NO:15A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15A. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:16A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16A.

In some embodiments, the intracellular costimulatory domain of the CD22 CAR comprises a 4-1BB costimulatory domain, for example, a human 4-1 BB costimulatory domain. In some embodiments, the 4-1BB costimulatory domain comprises or consists of an amino acid sequence set forth in SEQ ID NO:18A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18A. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:19A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:19A.

In some embodiments, the intracellular signaling domain of the CD22 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:20A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:20A.

In some embodiments, the second transgene comprises a nucleotide sequence encoding a CD22 CAR, including, for example, a CD22 CAR comprising the CD22-specific scFv having sequences set forth in SEQ ID NO:50A or SEQ ID NO:59A, the CD8α hinge domain of SEQ ID NO:9A, the CD8α transmembrane domain of SEQ ID NO:15A, the 4-1BB costimulatory domain of SEQ ID NO:18A, the CD3ζ signaling domain of SEQ ID NO:20A, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the second transgene comprises a nucleotide sequence encoding a CD22 CAR, including, for example, a CD22 CAR comprising the CD22-specific scFv having sequences set forth in SEQ ID NO:50A or SEQ ID NO:59A, the CD28 hinge domain of SEQ ID NO:10A, the CD8α transmembrane domain of SEQ ID NO:15A, the 4-1BB costimulatory domain of SEQ ID NO:18A, the CD3ζ signaling domain of SEQ ID NO:20A. and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the second transgene comprises a nucleotide sequence encoding a CD22 CAR, including, for example, a CD22 CAR comprising the CD22-specific scFv having sequences set forth in SEQ ID NO:50A or SEQ ID NO:59A, the IgG4 hinge domain of SEQ ID NO:12A or SEQ ID NO:13A, the CD8α transmembrane domain of SEQ ID NO:15A, the 4-1BB costimulatory domain of SEQ ID NO:18A, the CD3ζ signaling domain of SEQ ID NO:20A, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the second transgene comprises a nucleotide sequence encoding a CD22 CAR, including, for example, a CD22 CAR comprising the CD22-specific scFv having sequences set forth in SEQ ID NO:50A or SEQ ID NO:59A, the CD8α hinge domain of SEQ ID NO:9A, the CD28 transmembrane domain of SEQ ID NO:16A, the 4-1BB costimulatory domain of SEQ ID NO:18A, the CD3ζ signaling domain of SEQ ID NO:20A, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the second transgene comprises a nucleotide sequence encoding a CD22 CAR, including, for example, a CD22 CAR comprising the CD22-specific scFv having sequences set forth in SEQ ID NO:50A or SEQ ID NO:59A, the CD28 hinge domain of SEQ ID NO:10A, the CD28 transmembrane domain of SEQ ID NO:16A, the 4-1BB costimulatory domain of SEQ ID NO:18A, the CD3ζ signaling domain of SEQ ID NO:20A. and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the second transgene comprises a nucleotide sequence encoding a CD22 CAR, including, for example, a CD22 CAR comprising the CD22-specific scFv having sequences set forth in SEQ ID NO:50A or SEQ ID NO:59A, the IgG4 hinge domain of SEQ ID NO:12A or SEQ ID NO:13A, the CD28 transmembrane domain of SEQ ID NO:16A, the 4-1BB costimulatory domain of SEQ ID NO:18A, the CD3ζ signaling domain of SEQ ID NO:20A, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

### I. BCMA CAR

In some embodiments, the CAR is a BCMA CAR, and in these embodiments, the second transgene comprises a nucleotide sequence encoding a BCMA CAR. BCMA is a tumor necrosis family receptor (TNFR) member expressed on cells of the B cell lineage, with the highest expression on terminally differentiated B cells or mature B lymphocytes. BCMA is involved in mediating the survival of plasma cells for maintaining long-term humoral immunity. The expression of BCMA has been recently linked to a number of cancers, such as multiple myeloma, Hodgkin's and non-Hodgkin's lymphoma, various leukemias, and glioblastoma. In some embodiments, the BCMA CAR may comprise a signal peptide, an extracellular binding domain that specifically binds BCMA, a hinge domain, a transmembrane domain, an intracellular costimulatory domain, and/or an intracellular signaling domain in tandem.

In some embodiments, the signal peptide of the BCMA CAR comprises a CD8α signal peptide. In some embodiments, the CD8α signal peptide comprises or consists of an amino acid sequence set forth in SEQ ID NO:6A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6A. In some embodiments, the signal peptide comprises an IgK signal peptide. In some embodiments, the IgK signal peptide comprises or consists of an amino acid sequence set forth in SEQ ID NO:7A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7A. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8A.

In some embodiments, the extracellular binding domain of the BCMA CAR is specific to BCMA, for example, human BCMA. The extracellular binding domain of the BCMA CAR can be codon-optimized for expression in a host cell or to have variant sequences to increase functions of the extracellular binding domain.

In some embodiments, the extracellular binding domain comprises an immunogenically active portion of an immunoglobulin molecule, for example, an scFv. In some embodiments, the extracellular binding domain of the BCMA CAR is derived from an antibody specific to BCMA, including, for example, belantamab, erlanatamab, teclistamab, LCAR-B38M, and ciltacabtagene. In any of these embodiments, the extracellular binding domain of the BCMA CAR can comprise or consist of the V_{II}, the V_{L}, and/or one or more CDRs of any of the antibodies.

In some embodiments, the extracellular binding domain of the BCMA CAR comprises an scFv derived from C11D5.3, a murine monoclonal antibody as described in Carpenter et al., Clin. Cancer Res. 19(8):2048-2060 (2013). See also PCT Application Publication No. WO2010/104949. The C11D5.3-derived scFv may comprise the heavy chain variable region (V_{H}) and the light chain variable region (V_{L}) of C11D5.3 connected by the Whitlow linker, the amino acid sequences of which is provided in Table 15 below. In some embodiments, the BCMA-specific extracellular binding domain comprises or consists of an amino acid sequence set forth in SEQ ID NO:68A, 69A, or 73A, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:68A, 69A, or 73A. In some embodiments, the BCMA-specific extracellular binding domain may comprise one or more CDRs having amino acid sequences set forth in SEQ ID NOs: 70A, 71A, 72A and 74A, 75A, 76A. In some embodiments, the BCMA-specific extracellular binding domain may comprise a light chain with one or more CDRs having amino acid sequences set forth in SEQ ID NOs: 70A, 71A, 72A. In some embodiments, the BCMA-specific extracellular binding domain may comprise a heavy chain with one or more CDRs having amino acid sequences set forth in SEQ ID NOs: 74A, 75A, 76A. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from another murine monoclonal antibody, C12A3.2, as described in Carpenter et al., Clin. Cancer Res. 19(8):2048-2060 (2013) and PCT Application Publication No. WO2010/104949, the amino acid sequence of which is also provided in Table 15 below. In some embodiments, the BCMA-specific extracellular binding domain comprises or consists of an amino acid sequence set forth in SEQ ID NO:77A, 78A, or 82A, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:77A, 78A, or 82A. In some embodiments, the BCMA-specific extracellular binding domain may comprise one or more CDRs having amino acid sequences set forth in SEQ ID NOs: 79A, 80A, 81A and 83A, 84A, 85A. In some embodiments, the BCMA-specific extracellular binding domain may comprise a light chain with one or more CDRs having amino acid sequences set forth in SEQ ID NOs: 79A, 80A, 81A. In some embodiments, the BCMA-specific extracellular binding domain may comprise a heavy chain with one or more CDRs having amino acid sequences set forth in SEQ ID NOs: 83A, 84A, 85A. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises a murine monoclonal antibody with high specificity to human BCMA, referred to as BB2121 in Friedman et al., Hum. Gene Ther. 29(5):585-601 (2018)). See also, PCT Application Publication No. WO2012163805.

In some embodiments, the extracellular binding domain of the BCMA CAR comprises single variable fragments of two heavy chains (VHH) that can bind to two epitopes of BCMA as described in Zhao et al., J. Hematol. Oncol. 11(1):141 (2018), also referred to as LCAR-B38M. See also, PCT Application Publication No. WO2018/028647.

In some embodiments, the extracellular binding domain of the BCMA CAR comprises a fully human heavy-chain variable domain (FHVH) as described in Lam et al., Nat. Commun. 11(1):283 (2020), also referred to as FHVH33. See also, PCT Application Publication No. WO2019/006072. The amino acid sequences of FHVH33 and its CDRs are provided in Table 15 below. In some embodiments, the BCMA-specific extracellular binding domain comprises or consists of an amino acid sequence set forth in SEQ ID NO:86A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:86A. In some embodiments, the BCMA-specific extracellular binding domain may comprise one or more CDRs having amino acid sequences set forth in SEQ ID NOs: 87A, 88A, 89A. In any of these embodiments, the BCMA-specific extracellular binding domain may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from CT103A (or CAR0085) as described in U.S. Pat. No. 11,026,975 B2, the amino acid sequence of which is provided in Table 15 below. In some embodiments, the BCMA-specific extracellular binding domain comprises or consists of an amino acid sequence set forth in SEQ ID NO:90A, 91A, or 95A, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 90A, 91A, or 95A. In some embodiments, the BCMA-specific extracellular binding domain may comprise one or more CDRs having amino acid sequences set forth in SEQ ID NOs: 92A, 93A, 94A and 96A, 97A, 98A. In some embodiments, the BCMA-specific extracellular binding domain may comprise a light chain with one or more CDRs having amino acid sequences set forth in SEQ ID NOs: 92A, 93A, 94A. In some embodiments, the BCMA-specific extracellular binding domain may comprise a heavy chain with one or more CDRs having amino acid sequences set forth in SEQ ID NOs: 96A, 97A, 98A. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

Additionally, CARs and binders directed to BCMA have been described in U.S. Application Publication Nos. 2020/0246381 A1 and 2020/0339699 A1, the entire contents of each of which are incorporated by reference herein.

TABLE 15
Exemplary sequences of anti-BCMA binder and components

| SEQ ID NO: | Amino Acid Sequence | Description |
|---|---|---|
| 68A | DIVLTQSPASLAMSLGKRATISCRASESVSVIG | Anti-BCMA C11D5.3 |
| | AHLIHWYQQKPGQPPKLLIYLASNLETGVPAR | scFv entire sequence, |
| | FSGSGSGTDFTLTIDPVEEDDVAIYSCLQSRIFP | with Whitlow linker |
| | RTFGGGTKLEIKGSTSGSGKPGSGEGSTKGQIQ | |
| | LVQSGPELKKPGETVKISCKASGYTFTDYSIN | |
| | WVKRAPGKGLKWMGWINTETREPAYAYDFR | |
| | GRFAFSLETSASTAYLQINNLKYEDTATYFCA | |
| | LDYSYAMDYWGQGTSVTVSS | |
| 69A | DIVLTQSPASLAMSLGKRATISCRASESVSVIG | Anti-BCMA C11D5.3 |
| | AHLIHWYQQKPGQPPKLLIYLASNLETGVPAR | scFv light chain |
| | FSGSGSGTDFTLTIDPVEEDDVAIYSCLQSRIFP | variable region |
| | RTFGGGTKLEIK | |
| 70A | RASESVSVIGAHLIH | Anti-BCMA C11D5.3 |
| | | scFv light chain CDR1 |
| 71A | LASNLET | Anti-BCMA C11D5.3 |
| | | scFv light chain CDR2 |
| 72A | LQSRIFPRT | Anti-BCMA C11D5.3 |
| | | scFv light chain CDR3 |
| 73A | QIQLVQSGPELKKPGETVKISCKASGYTFTDYS | Anti-BCMA C11D5.3 |
| | INWVKRAPGKGLKWMGWINTETREPAYAYD | scFv heavy chain |
| | FRGRFAFSLETSASTAYLQINNLKYEDTATYFC | variable region |
| | ALDYSYAMDYWGQGTSVTVSS | |
| 74A | DYSIN | Anti-BCMA C11D5.3 |
| | | scFv heavy chain |
| | | CDR1 |
| 75A | WINTETREPAYAYDFRG | Anti-BCMA C11D5.3 |
| | | scFv heavy chain |
| | | CDR2 |
| 76A | DYSYAMDY | Anti-BCMA C11D5.3 |
| | | scFv heavy chain |
| | | CDR3 |
| 77A | DIVLTQSPPSLAMSLGKRATISCRASESVTILGS | Anti-BCMA C12A3.2 |
| | HLIYWYQQKPGQPPTLLIQLASNVQTGVPARF | scFv entire sequence, |
| | SGSGSRTDFTLTIDPVEEDDVAVYYCLQSRTIP | with Whitlow linker |
| | RTFGGGTKLEIKGSTSGSGKPGSGEGSTKGQIQ | |
| | LVQSGPELKKPGETVKISCKASGYTFRHYSMN | |
| | WVKQAPGKGLKWMGRINTESGVPIYADDFKG | |
| | RFAFSVETSASTAYLVINNLKDEDTASYFCSN | |
| | DYLYSLDFWGQGTALTVSS | |
| 78A | DIVLTQSPPSLAMSLGKRATISCRASESVTILGS | Anti-BCMA C12A3.2 |
| | HLIYWYQQKPGQPPTLLIQLASNVQTGVPARF | scFv light chain |
| | SGSGSRTDFTLTIDPVEEDDVAVYYCLQSRTIP | variable region |
| | RTFGGGTKLEIK | |
| 79A | RASESVTILGSHLIY | Anti-BCMA C12A3.2 |
| | | scFv light chain CDR1 |
| 80A | LASNVQT | Anti-BCMA C12A3.2 |
| | | scFv light chain CDR2 |
| 81A | LQSRTIPRT | Anti-BCMA C12A3.2 |
| | | scFv light chain CDR3 |
| 82A | QIQLVQSGPELKKPGETVKISCKASGYTFRHYS | Anti-BCMA C12A3.2 |
| | MNWVKQAPGKGLKWMGRINTESGVPIYADD | scFv heavy chain |
| | FKGRFAFSVETSASTAYLVINNLKDEDTASYF | variable region |
| | CSNDYLYSLDFWGQGTALTVSS | |
| 83A | HYSMN | Anti-BCMA C12A3.2 |
| | | scFv heavy chain |
| | | CDR1 |
| 84A | RINTESGVPIYADDFKG | Anti-BCMA C12A3.2 |
| | | scFv heavy chain |
| | | CDR2 |
| 85A | DYLYSLDF | Anti-BCMA C12A3.2 |
| | | scFv heavy chain |
| | | CDR3 |
| 86A | EVQLLESGGGLVQPGGSLRLSCAASGFTFSSY | Anti-BCMA FHVH33 |
| | AMSWVRQAPGKGLEWVSSISGSGDYIYYADS | entire sequence |
| | VKGRFTISRDISKNTLYLQMNSLRAEDTAVYY | |
| | CAKEGTGANSSLADYRGQGTLVTVSS | |
| 87A | GFTFSSYA | Anti-BCMA FHVH33 |
| | | CDR1 |
| 88A | ISGSGDYI | Anti-BCMA FHVH33 |
| | | CDR2 |
| 89A | AKEGTGANSSLADY | Anti-BCMA FHVH33 |
| | | CDR3 |
| 90A | DIQMTQSPSSLSASVGDRVTITCRASQSISSYLN | Anti-BCMA CT103A |
| | WYQQKPGKAPKLLIYAASSLQSGVPSRFSGSG | scFv entire sequence, |
| | SGTDFTLTISSLQPEDFATYYCQQKYDLLTFGG | with Whitlow linker |
| | GTKVEIKGSTSGSGKPGSGEGSTKGQLQLQES | |
| | GPGLVKPSETLSLTCTVSGGSISSSSYYWGWIR | |
| | QPPGKGLEWIGSISYSGSTYYNPSLKSRVTISV | |
| | DTSKNQFSLKLSSVTAADTAVYYCARDRGDTI | |
| | LDVWGQGTMVTVSS | |
| 91A | DIQMTQSPSSLSASVGDRVTITCRASQSISSYLN | Anti-BCMA CT103A |
| | WYQQKPGKAPKLLIYAASSLQSGVPSRFSGSG | scFv light chain |
| | SGTDFTLTISSLQPEDFATYYCQQKYDLLTFGG | variable region |
| | GTKVEIK | |
| 92A | QSISSY | Anti-BCMA CT103A |
| | | scFv light chain CDR1 |
| 93A | AAS | Anti-BCMA CT103A |
| | | scFv light chain CDR2 |
| 94A | QQKYDLLT | Anti-BCMA CT103A |
| | | scFv light chain CDR3 |
| 95A | QLQLQESGPGLVKPSETLSLTCTVSGGSISSSSY | Anti-BCMA CT103A |
| | YWGWIRQPPGKGLEWIGSISYSGSTYYNPSLK | scFv heavy chain |
| | SRVTISVDTSKNQFSLKLSSVTAADTAVYYCA | variable region |
| | RDRGDTILDVWGQGTMVTVSS | |
| 96A | GGSISSSSYY | Anti-BCMA CT103A |
| | | scFv heavy chain |
| | | CDR1 |
| 97A | ISYSGST | Anti-BCMA CT103A |
| | | scFv heavy chain |
| | | CDR2 |
| 98A | ARDRGDTILDV | Anti-BCMA CT103A |
| | | scFv heavy chain |
| | | CDR3 |

In some embodiments, the hinge domain of the BCMA CAR comprises a CD8α hinge domain, for example, a human CD8α hinge domain. In some embodiments, the CD8α hinge domain comprises or consists of an amino acid sequence set forth in SEQ ID NO:9A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9A. In some embodiments, the hinge domain comprises a CD28 hinge domain, for example, a human CD28 hinge domain. In some embodiments, the CD28 hinge domain comprises or consists of an amino acid sequence set forth in SEQ ID NO:10A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:10A. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:12A or SEQ ID NO:13A, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:12A or SEQ ID NO:13A. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:14A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:14A.

In some embodiments, the transmembrane domain of the BCMA CAR comprises a CD8α transmembrane domain, for example, a human CD8α transmembrane domain. In some embodiments, the CD8α transmembrane domain comprises or consists of an amino acid sequence set forth in SEQ ID NO:15A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15A. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:16A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16A.

In some embodiments, the intracellular costimulatory domain of the BCMA CAR comprises a 4-1BB costimulatory domain, for example, a human 4-1BB costimulatory domain. In some embodiments, the 4-1BB costimulatory domain comprises or consists of an amino acid sequence set forth in SEQ ID NO:18A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18A. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:19A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:19A.

In some embodiments, the intracellular signaling domain of the BCMA CAR comprises a CD3 zeta (ζ) signaling domain, for example, a human CD3ζ signaling domain. In some embodiments, the CD3ζ signaling domain comprises or consists of an amino acid sequence set forth in SEQ ID NO:20A or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:20A.

In some embodiments, the second transgene comprises a nucleotide sequence encoding a BCMA CAR, including, for example, a BCMA CAR comprising any of the BCMA-specific extracellular binding domains as described, the CD8α hinge domain of SEQ ID NO:9A, the CD8α transmembrane domain of SEQ ID NO:15A, the 4-1BB costimulatory domain of SEQ ID NO:18A, the CD3ζ signaling domain of SEQ ID NO:20A, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide (e.g., a CD8α signal peptide) as described.

In some embodiments, the second transgene comprises a nucleotide sequence encoding a BCMA CAR, including, for example, a BCMA CAR comprising any of the BCMA-specific extracellular binding domains as described, the CD8α hinge domain of SEQ ID NO:9A, the CD8α transmembrane domain of SEQ ID NO:15A, the CD28 costimulatory domain of SEQ ID NO:19A, the CD3ζ signaling domain of SEQ ID NO:20A, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide as described.

In some embodiments, the second transgene comprises a nucleotide sequence encoding a BCMA CAR as set forth in SEQ ID NO:99A or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:99A (see Table 16). The encoded BCMA CAR has a corresponding amino acid sequence set forth in SEQ ID NO:100A or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:10A, with the following components: CD8α signal peptide, CT103A scFv (V_{L}-Whitlow linker-V_{H}), CD8α hinge domain, CD8α transmembrane domain, 4-1BB costimulatory domain, and CD3 ζ signaling domain.

In some embodiments, the second transgene comprises a nucleotide sequence encoding a commercially available embodiment of BCMA CAR, including, for example, idecabtagene vicleucel (ide-cel, also called bb2121). In some embodiments, the second transgene comprises a nucleotide sequence encoding idecabtagene vicleucel or portions thereof. Idecabtagene vicleucel comprises a BCMA CAR with the following components: the BB2121 binder, CD8ai hinge domain, CD8α transmembrane domain, 4-1B costimulatory domain, and CD3ζ signaling domain.

TABLE 16
Exemplary sequences of BCMA CARs

| SEQ ID NO: | Sequence | Description |
|---|---|---|
| 99A | atggccttaccagtgaccgccttgctcctgccgctggccttgctgctccac | Exemplary BCMA |
| | gccgccaggccggacatccagatgacccagtctccatcctccctgtctgc | CAR nucleotide |
| | atctgtaggagacagagtcaccatcacttgccgggcaagtcagagcatta | sequence |
| | gcagctatttaaattggtatcagcagaaaccagggaaagcccctaagctc | |
| | ctgatctatgctgcatccagtttgcaaagtggggtcccatcaaggttcagtg | |
| | gcagtggatctgggacagatttcactctcaccatcagcagtctgcaacctg | |
| | aagattttgcaacttactactgtcagcaaaaatacgacctcctcacttttggc | |
| | ggagggaccaaggttgagatcaaaggcagcaccagcggctccggcaa | |
| | gcctggctctggcgagggcagcacaaagggacagctgcagctgcagga | |
| | glcgggcccaggactggtgaagccttcggagaccctgtccctcacctgca | |
| | ctgtctctggtggctccatcagcagtagtagttactactggggctggatccg | |
| | ccagcccccagggaaggggctggagtggattgggagtatctcctatagt | |
| | gggagcacctactacaacccgtccctcaagagtcgagtcaccatatccgt | |
| | agacacgtccaagaaccagttctccctgaagctgagttctgtgaccgccg | |
| | cagacacggcggtgtactactgcgccagagatcgtggagacaccatact | |
| | agacgtatggggtcagggtacaatggtcaccgtcagctcattcgtgcccgt | |
| | gttcctgcccgccaaacctaccaccacccctgcccctagacctcccaccc | |
| | cagccccaacaatcgccagccagcctctgtctctgcggcccgaagcctgt | |
| | agacctgctgccggcggagccgtgcacaccagaggcctggacttcgcct | |
| | gcgacatctacatctgggcccctctggccggcacctgtggcgtgctgctg | |
| | ctgagcctggtgatcaccctgtactgcaaccaccggaacaaacggggca | |
| | gaaagaaactcctgtatatattcaaacaaccatttatgagaccagtacaaac | |
| | tactcaagaggaagatggctgtagctgccgatttccagaagaagaagaag | |
| | gaggatgtgaactgagagtgaagttcagcagatccgccgacgcccctgc | |
| | ctaccagcagggacagaaccagctgtacaacgagctgaacctgggcag | |
| | acgggaagagtacgacgtgctggacaagcggagaggccgggaccccg | |
| | agatgggcggaaagcccagacggaagaacccccaggaaggcctgtata | |
| | acgaactgcagaaagacaagatggccgaggcctacagcgagatcggca | |
| | tgaagggcgagcggaggcgcggcaagggccacgatggcctgtaccag | |
| | ggcctgagcaccgccaccaaggacacctacgacgccctgcacatgcag | |
| | gccctgccccccaga | |
| 100A | MALPVTALLLPLALLLHAARPDIQMTQSPSSLS | Exemplary BCMA |
| | ASVGDRVTITCRASQSISSYLNWYQQKPGKAP | CAR amino acid |
| | KLLIYAASSLQSGVPSRFSGSGSGTDFTLTISSL | sequence |
| | QPEDFATYYCQQKYDLLTFGGGTKVEIKGSTS | |
| | GSGKPGSGEGSTKGQLQLQESGPGLVKPSETLS | |
| | LTCTVSGGSISSSSYYWGWIRQPPGKGLEWIGSI | |
| | SYSGSTYYNPSLKSRVTISVDTSKNQFSLKLSSV | |
| | TAADTAVYYCARDRGDTILDVWGQGTMVTVS | |
| | SFVPVFLPAKPTTTPAPRPPTP APTIASQPLSLRP | |
| | EACRPAAGGAVHTRGLDFACDIYIWAPLAGTC | |
| | GVLLLSLVITLYCNHRNKRGRKKLLYIFKQPFM | |
| | RPVQTTQEEDGCSCRFPEEEEGGCELRVKFSRS | |
| | ADAPAYQQGQNQLYNELNLGRREEYDVLDKR | |
| | RGRDPEMGGKPRRKNPQEGLYNELQKDKMAE | |
| | AYSEIGMKGERRRGKGHDGLYQGLSTATKDT | |
| | YDALHMQALPPR | |

### J. Multiple CARS

In some embodiments, the second transgene comprises two or more nucleotide sequences, each encoding a CAR targeting a specific target antigen. In these embodiments, the second transgene encodes two or more different CARs specific to different target antigens (e.g., a CD19 CAR and a CD22 CAR). The two or more CARs may each comprise an extracellular binding domain specific to a specific target antigen, and may comprise the same, or one or more different, non-antigen binding domains. For example, the two or more CARs may comprise different signal peptides, hinge domains, transmembrane domains, costimulatory domains, and/or intracellular signaling domains, in order to minimize the risk of recombination due to sequence similarities. Or, alternatively, the two or more CARs may comprise the same non-antigen binding domains. In the cases where the same non-antigen binding domain(s) and/or backbone are used, it is optional to introduce codon divergence at the nucleotide sequence level to minimize the risk of recombination. As one non-limiting example, the second transgene may comprise a nucleotide sequence encoding a CD19 CAR and a nucleotide sequence encoding a CD22 CAR. The CD19 CAR may comprise one transmembrane domain (e.g., CD28 transmembrane domain) while the CD22 CAR comprises a different transmembrane domain (e.g., CD8α transmembrane domain), or vice versa. As another non-limiting example, the CD19 CAR may comprise one costimulatory domain (e.g., 4-1BB costimulatory domain) while the CD22 CAR comprises a different costimulatory domain (e.g., CD28 costimulatory domain), or vice versa. Or, alternatively, the CD22 CAR and the CD19 CARs may comprise the same non-antigen binding domains but have codon divergence introduced at the nucleotide sequence level to minimize the risk of recombination. In any of these embodiments, the two or more nucleotide sequences of the second transgene may be connected by one or more cleavage sites as described (e.g., a 2A site and/or a furin site), in the form of polycistronic constructs as described herein.

### 3. Regulatory Elements

In some embodiments, the second transgene encoding a CAR may comprise additional regulatory elements operatively linked to the CAR encoding sequence as described, including, for example, promoters, insulators, enhancers, polyadenylation (poly(A)) tails, and/or ubiquitous chromatin opening elements.

### 4. Genomic Insertion

In some embodiments, the second transgene encoding a CAR may be delivered into a host cell in the form of a vector for insertion into the host genome. The insertion may be random (i.e., insertion into a random genomic locus of the host cell) or targeted (i.e., insertion into a specific genomic locus of the host cell), using any of the random or site-directed insertion methods described herein.

In some embodiments, the first transgene encoding a tolerogenic factor and the second transgene encoding a CAR may be introduced into a host for genomic insertion separately. In some embodiments, the first transgene encoding a tolerogenic factor and the second transgene encoding a CAR may be introduced into a host for genomic insertion at the same time, via a single vector or multiple vectors. In cases where the first and the second transgene are delivered into a host cell together in a single vector, the first and the second transgene may be designed as a polycistronic construct as described below.

### 5. Polycistronic Constructs

In some embodiments, the first transgene encoding a tolerogenic factor and the second transgene encoding a CAR, and/or the multiple CAR encoding sequences of the second transgene, may be in the form of polycistronic constructs. Polycistronic constructs have two or more expression cassettes for co-expression of two or more proteins of interest in a host cell. In some embodiments, the polycistronic construct comprises two expression cassettes, i.e., is bicistronic. In some embodiments, the polycistronic construct comprises three expression cassettes, i.e., is tricistronic. In some embodiments, the polycistronic construct comprises four expression cassettes, i.e., is quadcistronic. In some embodiments, the polycistronic construct comprises more than four expression cassettes. In any of these embodiments, each of the expression cassettes comprises a nucleotide sequence encoding a protein of interest (e.g., a tolerogenic or a CAR). In certain embodiments, the two or more genes being expressed are under the control of a single promoter and are separated from one another by one or more cleavage sites to achieve co-expression of the proteins of interest from one transcript. In other embodiments, the two or more genes may be under the control of separate promoters.

In some embodiments, the two or more expression cassettes of the polycistronic construct may be separated by one or more cleavage sites. As the name suggests, a polycistronic construct allows simultaneous expression of two or more separate proteins from one mRNA transcript in a host cell. Cleavage sites can be used in the design of a polycistronic construct to achieve such co-expression of multiple genes.

In some embodiments, the one or more cleavage sites comprise one or more self-cleaving sites. In some embodiments, the self-cleaving site comprises a 2A site. 2A peptides are a class of 18-22 amino acid-long peptides first discovered in picornaviruses and can induce ribosomal skipping during translation of a protein, thus producing equal amounts of multiple genes from the same mRNA transcript. 2A peptides function to “cleave” an mRNA transcript by making the ribosome skip the synthesis of a peptide bond at the C-terminus, between the glycine (G) and proline (P) residues, leading to separation between the end of the 2A sequence and the next peptide downstream. There are four 2A peptides commonly employed in molecular biology, T2A. P2A. E2A, and F2A, the sequences of which are summarized in Table 19. A glycine-serine-glycine (GSG) linker is optionally added to the N-terminal of a 2A peptide to increase cleavage efficiency. The use of “( )” around a sequence in the present disclosure means that the enclosed sequence is optional.

TABLE 19
Sequences of 2A peptides

| SEQ | | 2A |
|---|---|---|
| ID NO: | Amino Acid Sequence | Peptide |
| 101A | (GSG)EGRGSLLTCGDVEENPGP | T2A |
| 102A | (GSG)ATNFSLLKQAGDVEENPGP | P2A |
| 103A | (GSG)QCTNYALLKLAGDVESNPGP | E2A |
| 104A | (GSG)VKQTLNFDLLKLAGDVESNPGP | F2A |

In some embodiments, the one or more cleavage sites additionally comprise one or more protease sites. The one or more protease sites can either precede or follow the self-cleavage sites (e.g., 2A sites) in the 5′ to 3′ order. The protease site may be cleaved by a protease after translation of the full transcript or after translation of each expression cassette such that the first expression product is released prior to translation of the next expression cassette. In these embodiments, having a protease site in addition to the 2A site, especially preceding the 2A site in the 5′ to 3′ order, may reduce the number of extra amino acid residues attached to the expressed proteins of interest. In some embodiments, the protease site comprises a furin site, also known as a Paired basic Amino acid Cleaving Enzyme (PACE) site. There are at least three furin cleavage sequences, FC1. FC2, and FC3, the amino acid sequences of which are summarized in Table 20. Similar to the 2A sites, one or more optional glycine-serine-glycine (GSG) sequences can be included for cleavage efficiency.

TABLE 20
Sequences of furin sites

| SEQ ID NO: | Amino Acid Sequence | Furin site |
|---|---|---|
| 105A | RRRR (GSG) | FC1 |
| 106A | RKRR (GSG) | FC2 |
| 107A | RKRR (GSG) TPDPW (GSG) | FC3 |

In some embodiments, the one or more cleavage sites comprise one or more self-cleaving sites, one or more protease sites, and/or any combination thereof. For example, the cleavage site can include a 2A site alone. For another example, the cleavage site can include a FC2 or FC3 site, followed by a 2A site. In these embodiments, the one or more self-cleaving sites may be the same or different. Similarly, the one or more protease sites may be the same or different.

In some embodiments, the polycistronic construct may be in the form of a vector. Any type of vector suitable for introduction of nucleotide sequences into a host cell can be used, including, for example, plasmids, adenoviral vectors, adenoviral-associated vectors, retroviral vectors, lentiviral vectors, phages, and homology-directed repair (HDR)-based donor vectors.

### 6. Methods of Increasing Expression of (e.g., Overexpressing) a Polynucleotide

In some embodiments, increased expression of a polynucleotide may be carried out by any of a variety of techniques. For instance, methods for modulating expression of genes and factors (proteins) include genome editing technologies, and, RNA or protein expression technologies and the like. For all of these technologies, well known recombinant techniques are used, to generate recombinant nucleic acids as outlined herein. In some embodiments, the cell that is engineered with the one or more modification for overexpression or increased expression of a polynucleotide is any source cell as described herein. In some embodiments, the source cell is any cell described in Section II.C.

In some embodiments, expression of a gene is increased by increasing endogenous gene activity (e.g., increasing transcription of the exogenous gene). In some cases, endogenous gene activity is increased by increasing activity of a promoter or enhancer operably linked to the endogenous gene. In some embodiments, increasing activity of the promoter or enhancer comprises making one or more modifications to an endogenous promoter or enhancer that increase activity of the endogenous promoter or enhancer. In some cases, increasing gene activity of an endogenous gene comprises modifying an endogenous promoter of the gene. In some embodiments increasing gene activity of an endogenous gene comprises introducing a heterologous promoter. In some embodiments, the heterologous promoter is selected from the group consisting of a CAG promoter, cytomegalovirus (CMV) promoter, EF 1a promoter, PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein Barr virus (EBV) promoter, Rous sarcoma virus (RSV) promoter, and UBC promoter.

In some embodiments, expression of a target gene (e.g., CD47, or another tolerogenic factor) is increased by expression of fusion protein or a protein complex containing (1) a site-specific binding domain specific for the endogenous CD47, or other gene and (2) a transcriptional activator.

In some embodiments, the regulatory factor is comprised of a site specific DNA-binding nucleic acid molecule, such as a guide RNA (gRNA). In some embodiments, the method is achieved by site specific DNA-binding targeted proteins, such as zinc finger proteins (ZFP) or fusion proteins containing ZFP, which are also known as zinc finger nucleases (ZFNs).

In some embodiments, the regulatory factor comprises a site-specific binding domain, such as using a DNA binding protein or DNA-binding nucleic acid, which specifically binds to or hybridizes to the gene at a targeted region. In some embodiments, the provided polynucleotides or polypeptides are coupled to or complexed with a site-specific nuclease, such as a modified nuclease. For example, in some embodiments, the administration is effected using a fusion comprising a DNA-targeting protein of a modified nuclease, such as a meganuclease or an RNA-guided nuclease such as a clustered regularly interspersed short palindromic nucleic acid (CRISPR)-Cas system, such as CRISPR-Cas9 system. In some embodiments, the nuclease is modified to lack nuclease activity. In some embodiments, the modified nuclease is a catalytically dead dCas9.

In some embodiments, the site specific binding domain may be derived from a nuclease. For example, the recognition sequences of homing endonucleases and meganucleases such as I-SceI, I-CeuI, PI-PspI, PI-Sce, I-SceIV, I-CsmI, I-PanI, I-SceII, I-PpoI, I-SceIII, I-CreI, I-TevI. I-TevlI and I-TevIII. See also U.S. Pat. Nos. 5,420,032; 6,833,252; Belfort et al. , (1997) Nucleic Acids Res. 25:3379-3388; Dujon et al., (1989) Gene 82:115-118; Perler et al, (1994) Nucleic Acids Res. 22, 1125-1127; Jasin (1996) Trends Genet. 12:224-228; Gimble et al., (1996) J. Mol. Biol. 263:163-180; Argast et al. (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al, (2002) Molec. Cell 10:895-905; Epinat et al, (2003) Nucleic Acids Res. 31:2952-2962: Ashworth et al, (2006) Nature 441:656-659; Paques et al, (2007) Current Gene Therapy 7:49-66; U.S. Patent Publication No. 2007/0117128.

Zinc finger. TALE, and CRISPR system binding domains can be “engineered” to bind to a predetermined nucleotide sequence, for example via engineering (altering one or more amino acids) of the recognition helix region of a naturally occurring zinc finger or TALE protein. Engineered DNA binding proteins (zinc fingers or TALEs) are proteins that are non-naturally occurring. Rational criteria for design include application of substitution rules and computerized algorithms for processing information in a database storing information of existing ZFP and/or TALE designs and binding data. See, for example, U.S. Pat. Nos. 6,140,081; 6.453,242; and 6,534,261; see also WO 98/53058; WO 98/53059; WO 98/53060; WO 02/016536 and WO 03/016496 and U.S. Publication No. 20110301073.

In some embodiments, the site-specific binding domain comprises one or more zinc-finger proteins (ZFPs) or domains thereof that bind to DNA in a sequence-specific manner. A ZFP or domain thereof is a protein or domain within a larger protein that binds DNA in a sequence-specific manner through one or more zinc fingers, regions of amino acid sequence within the binding domain whose structure is stabilized through coordination of a zinc ion.

Among the ZFPs are artificial ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to a target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416: U.S. Pat. Nos. 6,453,242: 6,534,261: 6,599,692: 6,503,717; 6,689.558; 7,030,215; 6,794.136: 7,067,317: 7,262,054; 7,070,934; 7,361.635; 7,253,273; and U.S. Patent Publication Nos. 2005/0064474; 2007/0218528; 2005/0267061, all incorporated herein by reference in their entireties.

Many gene-specific engineered zinc fingers are available commercially. For example, Sangamo Biosciences (Richmond, CA, USA) has developed a platform (CompoZr) for zinc-finger construction in partnership with Sigma-Aldrich (St. Louis, MO, USA), allowing investigators to bypass zinc-finger construction and validation altogether, and provides specifically targeted zinc fingers for thousands of proteins (Gaj et al., Trends in Biotechnology. 2013, 31(7). 397-405). In some embodiments, commercially available zinc fingers are used or are custom designed.

In some embodiments, the site-specific binding domain comprises a naturally occurring or engineered (non-naturally occurring) transcription activator-like protein (TAL) DNA binding domain, such as in a transcription activator-like protein effector (TALE) protein, See, e.g., U.S. Patent Publication No. 20110301073, incorporated by reference in its entirety herein.

In some embodiments, the site-specific binding domain is derived from the CRISPR/Cas system. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system, or a “targeting sequence”), and/or other sequences and transcripts from a CRISPR locus.

In general, a guide sequence includes a targeting domain comprising a polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. In some examples, the targeting domain (e.g., targeting sequence) of the gRNA is complementary, e.g., at least 80, 85, 90, 95, 98 or 99% complementary, e.g., fully complementary, to the target sequence on the target nucleic acid.

In some embodiments, the gRNA may be any as described herein. In embodiments, the gRNA has a targeting sequence that is complementary to a target site of CD47, such as set forth in any one of SEQ ID NOS:200784-231885 (Table 29, Appendix 22 of WO2016183041); HLA-E, such as set forth in any one of SEQ ID NOS:189859-193183 (Table 19. Appendix 12 of WO2016183041); HLA-F, such as set forth in any one of SEQ ID NOS: 688808-699754 (Table 45. Appendix 38 of WO2016183041); HLA-G, such as set forth in any one of SEQ ID NOS:188372-189858 (Table 18, Appendix 11 of WO2016183041); or PD-L1, such as set forth in any one of SEQ ID NOS: 193184-200783 (Table 21, Appendix 14 of WO2016183041).

In some embodiments, the target site is upstream of a transcription initiation site of the target gene. In some embodiments, the target site is adjacent to a transcription initiation site of the gene. In some embodiments, the target site is adjacent to an RNA polymerase pause site downstream of a transcription initiation site of the gene.

In some embodiments, the targeting domain is configured to target the promoter region of the target gene to promote transcription initiation, binding of one or more transcription enhancers or activators, and/or RNA polymerase. One or more gRNA can be used to target the promoter region of the gene. In some embodiments, one or more regions of the gene can be targeted. In certain aspects, the target sites are within 600 base pairs on either side of a transcription start site (TSS) of the gene.

It is within the level of a skilled artisan to design or identify a gRNA sequence that is or comprises a sequence targeting a gene (i.e. gRNA targeting sequence), including the exon sequence and sequences of regulatory regions, including promoters and activators. A genome-wide gRNA database for CRISPR genome editing is publicly available, which contains exemplary single guide RNA (sgRNA) target sequences in constitutive exons of genes in the human genome or mouse genome (see e.g., genescript.com/gRNA-database.html; see also, Sanjana et al. (2014) Nat. Methods, 11:783-4; www.e-crisp.org/E-CRISP/; crispr.mit.edu/). In some embodiments, the gRNA sequence is or comprises a targeting sequence with minimal off-target binding to a non-target gene.

In some embodiments, the regulatory factor further comprises a functional domain, e.g., a transcriptional activator.

In some embodiments, the transcriptional activator is or contains one or more regulatory elements, such as one or more transcriptional control elements of a target gene, whereby a site-specific domain as provided above is recognized to drive expression of such gene. In some embodiments, the transcriptional activator drives expression of the target gene. In some cases, the transcriptional activator, can be or contain all or a portion of a heterologous transactivation domain. For example, in some embodiments, the transcriptional activator is selected from Herpes simplex-derived transactivation domain, Dnmt3a methyltransferase domain, p65, VP16, and VP64.

In some embodiments, the regulatory factor is a zinc finger transcription factor (ZF-TF). In some embodiments, the regulatory factor is VP64-p65-Rta (VPR).

In certain embodiments, the regulatory factor further comprises a transcriptional regulatory domain. Common domains include. e.g., transcription factor domains (activators, repressors, co-activators, co-repressors), silencers, oncogenes (e.g., myc, jun, fos, myb, max, mad, rel, ets, bcl, myb, mos family members etc.); DNA repair enzymes and their associated factors and modifiers; DNA rearrangement enzymes and their associated factors and modifiers; chromatin associated proteins and their modifiers (e.g., kinases, acetylases and deacetylases); and DNA modifying enzymes (e.g., methyltransferases such as members of the DNMT family (e.g., DNMT1, DNMT3A, DNMT3B, DNMT3L, etc., topoisomerases, helicases, ligases, kinases, phosphatases, polymerases, endonucleases) and their associated factors and modifiers. See, e.g., U.S. Publication No. 2013/0253040, incorporated by reference in its entirety herein.

Suitable domains for achieving activation include the HSV VP 16 activation domain (see, e.g., Hagmann et al, J. Virol. 71, 5952-5962 (1 97)) nuclear hormone receptors (see, e.g., Torchia et al., Curr. Opin. Cell. Biol. 10:373-383 (1998)); the p65 subunit of nuclear factor kappa B (Bitko & Bank. J. Virol. 72:5610-5618 (1998) and Doyle & Hunt. Neuroreport 8:2937-2942 (1997)); Liu et al., Cancer Gene Ther. 5:3-28 (1998)), or artificial chimeric functional domains such as VP64 (Beerli et al., (1998) Proc. Natl. Acad. Sci. USA 95:14623-33), and degron (Molinari et al., (1999) EMBO J. 18, 6439-6447). Additional exemplary activation domains include, Oct 1, Oct-2A, Sp1, AP-2, and CTF1 (Seipel etal, EMBOJ. 11, 4961-4968 (1992) as well as p300, CBP, PCAF, SRC1 PvALF, AtHD2A and ERF-2. See, for example, Robyr et al, (2000) Mol. Endocrinol. 14:329-347; Collingwood et al, (1999) J. Mol. Endocrinol 23:255-275; Leo et al, (2000) Gene 245:1-11; Manteuffel-Cymborowska (1999) Acta Biochim. Pol. 46:77-89; McKenna et al, (1999) J. Steroid Biochem. Mol. Biol. 69:3-12; Malik et al, (2000) Trends Biochem. Sci. 25:277-283; and Lemon et al, (1999) Curr. Opin. Genet. Dev. 9:499-504. Additional exemplary activation domains include, but are not limited to, OsGAI, HALF-1, C1, AP1, ARF-5, -6,-1, and -8, CPRF1, CPRF4, MYC-RP/GP, and TRAB1, See, for example, Ogawa et al, (2000) Gene 245:21-29; Okanami et al, (1996) Genes Cells 1:87-99: Goff et al, (1991) Genes Dev. 5:298-309: Cho et al, (1999) Plant Mol Biol 40:419-429; Ulmason et al. (1999) Proc. Natl. Acad. Sci. USA 96:5844-5849; Sprenger-Haussels et al, (2000) Plant J. 22:1-8; Gong et al, (1999) Plant Mol. Biol. 41:33-44; and Hobo et al. , (1999) Proc. Natl. Acad. Sci. USA 96:15,348-15,353.

Exemplary repression domains that can be used to make genetic repressors include, but are not limited to, KRAB A/B, KOX, TGF-beta-inducible early gene (TIEG), v-erbA, SID, MBD2, MBD3, members of the DNMT family (e.g., DNMT1, DNMT3A, DNMT3B, DNMT3L, etc.), Rb, and MCCP2. See, for example, Bird et al, (1999) Cell 99:451-454; Tyler et al, (1999) Cell 99:443-446; Knoepfler et al, (1999) Cell 99:447-450; and Robertson et al, (2000) Nature Genet. 25:338-342. Additional exemplary repression domains include, but are not limited to, ROM2 and AtHD2A. See, for example, Chem et al. (1996) Plant Cell 8:305-321; and Wu et al, (2000) Plant J. 22:19-27.

In some instances, the domain is involved in epigenetic regulation of a chromosome. In some embodiments, the domain is a histone acetyltransferase (HAT). e.g., type- A, nuclear localized such as MYST family members MOZ. Ybf2/Sas3, MOF, and Tip60, GNAT family members Gcn5 or pCAF, the p300 family members CBP, p300 or Rtt109 (Bemdsen and Denu (2008) Curr Opin Struct Biol 18(6):682-689). In other instances the domain is a histone deacetylase (HD AC) such as the class I (HDAC-1, 2, 3, and 8), class II (HDAC IIA (HDAC-4, 5, 7 and 9), HD AC IIB (HDAC 6 and 10)), class IV (HDAC-11), class III (also known as sirtuins (SIRTs); SIRT1-7) (see Mottamal et al., (2015) Molecules 20(3):3898-3941). Another domain that is used in some embodiments is a histone phosphorylase or kinase, where examples include MSK1, MSK2, ATR, ATM, DNA-PK, Bub1, VprBP, IKK-a, PKCpi, Dik/Zip, JAK2, PKC5, WSTF and CK2. In some embodiments, a methylation domain is used and may be chosen from groups such as Ezh2, PRMT1/6, PRMT5/7, PRMT 2/6, CARM1, set7/9, MLL, ALL-1, Suv 39h, G9a, SETDB1, Ezh2, Set2, Dot1, PRMT 1/6, PRMT 5/7, PR-Set7 and Suv4-20h, Domains involved in sumoylation and biotinylation (Lys9, 13, 4, 18 and 12) may also be used in some embodiments (review see Kousarides (2007) Cell 128:693-705).

Fusion molecules are constructed by methods of cloning and biochemical conjugation that are well known to those of skill in the art. Fusion molecules comprise a DNA-binding domain and a functional domain (e.g., a transcriptional activation or repression domain). Fusion molecules also optionally comprise nuclear localization signals (such as, for example, that from the SV40 medium T-antigen) and epitope tags (such as, for example, FLAG and hemagglutinin). Fusion proteins (and nucleic acids encoding them) are designed such that the translational reading frame is preserved among the components of the fusion.

Fusions between a polypeptide component of a functional domain (or a functional fragment thereof) on the one hand, and a non-protein DNA-binding domain (e.g., antibiotic, intercalator, minor groove binder, nucleic acid) on the other, are constructed by methods of biochemical conjugation known to those of skill in the art. See, for example, the Pierce Chemical Company (Rockford, IL) Catalogue. Methods and compositions for making fusions between a minor groove binder and a polypeptide have been described. Mapp et al, (2000) Proc. Natl. Acad. Sci. USA 97:3930-3935. Likewise, CRISPR/Cas TFs and nucleases comprising a sgRNA nucleic acid component in association with a polypeptide component function domain are also known to those of skill in the art and detailed herein.

### A. Exogenous Polypeptide

In some embodiments, increased expression (i.e. overexpression) of the polynucleotide is mediated by introducing into the cell an exogenous polynucleotide encoding the polynucleotide to be overexpressed. In some embodiments, the exogenous polynucleotide is a recombinant nucleic acid. Well-known recombinant techniques can be used to generate recombinant nucleic acids as outlined herein. In some embodiments, an exogenous polynucleotide encoding an exogenous polypeptide herein comprises a codon-optimized nucleic acid sequence.

In certain embodiments, the recombinant nucleic acids encoding an exogenous polypeptide, such as a tolerogenic factor or a chimeric antigen receptor, may be operably linked to one or more regulatory nucleotide sequences in an expression construct. Regulatory nucleotide sequences will generally be appropriate for the host cell and recipient subject to be treated. Numerous types of appropriate expression vectors and suitable regulatory sequences are known in the art for a variety of host cells. Typically, the one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are also contemplated. The promoters may be either naturally occurring promoters, or hybrid promoters that combine elements of more than one promoter. An expression construct may be present in a cell on an episome, such as a plasmid, or the expression construct may be inserted in a chromosome. In some embodiments, the expression vector includes a selectable marker gene to allow the selection of transformed host cells. Certain embodiments include an expression vector comprising a nucleotide sequence encoding a variant polypeptide operably linked to at least one regulatory sequence. Regulatory sequence for use herein include promoters, enhancers, and other expression control elements. In certain embodiments, an expression vector is designed for the choice of the host cell to be transformed, the particular variant polypeptide desired to be expressed, the vector's copy number, the ability to control that copy number, and/or the expression of any other protein encoded by the vector, such as antibiotic markers.

In some embodiments, the exogenous polynucleotide is operably linked to a promoter for expression of the exogenous polynucleotide in the engineered cell. Examples of suitable mammalian promoters include, for example, promoters from the following genes: elongation factor 1 alpha (EF1α) promoter, ubiquitin/S27a promoter of the hamster (WO 97/15664). Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus Long Terminal repeat region, and the early promoter of human Cytomegalovirus (CMV). Examples of other heterologous mammalian promoters are the actin, immunoglobulin or heat shock promoter(s). In additional embodiments, promoters for use in mammalian host cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication (Fiers et al. Nature 273: 113-120 (1978)). The immediate early promoter of the human cytomegalovirus is conveniently obtained as a HindIII restriction enzyme fragment (Greenaway et al, Gene 18: 355-360 (1982)). The foregoing references are incorporated by reference in their entirety.

In some embodiments, the expression vector is a bicistronic or multicistronic expression vector. Bicistronic or multicistronic expression vectors may include (1) multiple promoters fused to each of the open reading frames; (2) insertion of splicing signals between genes; (3) fusion of genes whose expressions are driven by a single promoter; and/or (4) insertion of proteolytic cleavage sites between genes (self-cleavage peptide) or insertion of internal ribosomal entry sites (IRESs) between genes.

In some embodiments, an expression vector or construct herein is a multicistronic construct. The terms “multicistronic construct” and “multicistronic vector” are used interchangeably herein and refer to a recombinant DNA construct that is to be transcribed into a single mRNA molecule, wherein the single mRNA molecule encodes two or more genes (e.g., two or more transgenes). The multicistronic construct is referred to as bicistronic construct if it encodes two genes, and tricistronic construct if it encodes three genes, and quadrocistronic construct if it encodes four genes, and so on.

In some embodiments, two or more exogenous polynucleotides comprised by a vector or construct (e.g., a transgene) are each separated by a multicistronic separation element. In some embodiments, the multicistronic separation element is an IRES or a sequence encoding a cleavable peptide or ribosomal skip element. In some embodiments, the multicistronic separation element is an IRES, such as an encephalomyocarditis (EMCV) virus IRES. In some embodiments, the multicistronic separation element is a cleavable peptide such as a 2A peptide. Exemplary 2A peptides include a P2A peptide, a T2A peptide, an E2A peptide, and an F2Apeptide. In some embodiments, the cleavable peptide is a T2A. In some embodiments, the two or more exogenous polynucleotides (e.g., the first exogenous polynucleotide and second exogenous polynucleotide) are operably linked to a promoter. In some embodiments, the first exogenous polynucleotide and the second exogenous polynucleotide are each operably linked to a promoter. In some embodiments, the promoter is the same promoter. In some embodiments, the promoter is an EF1 promoter.

In some cases, an exogenous polynucleotide encoding an exogenous polypeptide (e.g., an exogenous polynucleotide encoding a tolerogenic factor or complement inhibitor described herein) encodes a cleavable peptide or ribosomal skip element, such as T2A at the N-terminus or C-terminus of an exogenous polypeptide encoded by a multicistronic vector. In some embodiments, inclusion of the cleavable peptide or ribosomal skip element allows for expression of two or more polypeptides from a single translation initiation site. In some embodiments, the cleavable peptide is a T2A. In some embodiments, the T2A is or comprises the amino acid sequence set forth by SEQ ID NO: 11. In some embodiments, the T2A is or comprises the amino acid sequence set forth by SEQ ID NO: 12. In some embodiments, the T2A is or comprises the amino acid sequence set forth by SEQ ID NO: 17. In some embodiments, the T2A is or comprises the amino acid sequence set forth by SEQ ID NO: 18.

In some embodiments, the vector or construct includes a single promoter that drives the expression of one or more transcription units of an exogenous polynucleotide. In some embodiments, such vectors or constructs can be multicistronic (bicistronic or tricistronic, see e.g., U.S. Pat. No. 6,060,273). For example, in some embodiments, transcription units can be engineered as a bicistronic unit containing an IRES (internal ribosome entry site), which allows coexpression of gene products (e.g., one or more tolerogenic factor such as CD47 and/or one or more complement inhibitor such as CD46, CD59, and CD55) from an RNA transcribed from a single promoter. In some embodiments, the vectors or constructs provided herein are bicistronic, allowing the vector or construct to express two separate polypeptides. In some cases, the two separate polypeptides encoded by the vector or construct are tolerogenic factors (e.g., two factors selected from CD16, CD24, CD35, CD39, CD46, CD47, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/INFAIP3, CR1, HLA-F, MANF). In some cases, the two separate polypeptides encoded by the vector or construct are CD46 and CD59. In some embodiments, the two separate polypeptides encoded by the vector or construct are a tolerogenic factor (e.g., CD47) and a complement inhibitor selected from CD46, CD59, and CD55. In some embodiments, the vectors or constructs provided herein are tricistronic, allowing the vector or construct to express three separate polypeptides. In some cases, the three nucleic acid sequences of the tricistronic vector or construct are a tolerogenic factor such as CD47, CD46, and CD59. In some cases, the three nucleic acid sequences of the tricistronic vector or construct are CD46, CD59, and CD55. In some cases, the three nucleic acid sequences of the tricistronic vector or construct are three tolerogenic factors selected from CD16, CD24, CD35, CD39, CD46, CD47, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, MANF. In some embodiments, the vectors or constructs provided herein are quadrocistronic, allowing the vector or construct to express four separate polypeptides. In some cases, the four separate polypeptides of the quadrocistronic vector or construct are CD47, CD46, CD59, and CD55. In some cases, the four separate polypeptides of the quadrocistronic vector or construct are four tolerogenic factors selected from CD16, CD24, CD35, CD39, CD46, CD47, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G). A20/TNFAIP3, CR1, HLA-F, MANF.

In some embodiments, the cell comprises one or more vectors or constructs, wherein each vector or construct is a monocistronic or a multicistronic construct as described above, and the monocistronic or multicistronic constructs encode one or more tolerogenic factors, complement inhibitors in any combination or order.

In some embodiments, a single promoter directs expression of an RNA that contains, in a single open reading frame (ORF), two, three, or four genes (e.g., encoding a tolerogenic factor (e.g., CD47) and/or one or more complement inhibitors selected from CD46, CD59, and CD55) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known in the art. Examples of 2A sequences that can be used in the methods and nucleic acids disclosed herein include, without limitation, 2A sequences from the foot-and-mouth disease virus (F2A, e.g., SEQ ID NO: 16), equine rhinitis A virus (E2A, e.g., SEQ ID NO: 15), thosea asigna virus (T2A, e.g., SEQ ID NO: 11, 12, 17, or 18), and porcine teschovirus-1 (P2A, e.g., SEQ ID NO: 13 or 14) as described in U.S. Patent Publication No. 20070116690.

In cases where the vector or construct (e.g., transgene) contains more than one nucleic acid sequence encoding a protein, e.g., a first exogenous polynucleotide encoding CD46 and a second exogenous polynucleotide encoding CD59, or a first exogenous polynucleotide encoding CD47, a second exogenous polynucleotide encoding CD56, and a third exogenous polynucleotide encoding CD59, the vector or construct (e.g., transgene) may further include a nucleic acid sequence encoding a peptide between the first and second exogenous polynucleotide sequences. In some cases, the nucleic acid sequence positioned between the first and second exogenous polynucleotides encodes a peptide that separates the translation products of the first and second exogenous polynucleotides during or after translation. In some embodiments, the peptide contains a self-cleaving peptide or a peptide that causes ribosome skipping (a ribosomal skip element), such as a T2A peptide. In some embodiments, inclusion of the cleavable peptide or ribosomal skip element allows for expression of two or more polypeptides from a single translation initiation site. In some embodiments, the peptide is a self-cleaving peptide that is a T2A peptide. In some embodiments, the T2A is or comprises the amino acid sequence set forth by SEQ ID NO: 11. In some embodiments, the T2A is or comprises the amino acid sequence set forth by SEQ ID NO: 12. In some embodiments, the T2A is or comprises the amino acid sequence set forth by SEQ ID NO: 17. In some embodiments, the T2A is or comprises the amino acid sequence set forth by SEQ ID NO: 18.

The process of introducing the polynucleotides described herein into cells can be achieved by any suitable technique. Suitable techniques include calcium phosphate or lipid-mediated transfection, electroporation, fusogens, and transduction or infection using a viral vector. In some embodiments, the polynucleotides are introduced into a cell via viral transduction (e.g., lentiviral transduction) or otherwise delivered on a viral vector (e.g., fusogen-mediated delivery). Suitable techniques include calcium phosphate or lipid-mediated transfection, electroporation, transposase-mediated delivery, and transduction or infection using a viral vector. In some embodiments, the polynucleotides are introduced into a cell via viral transduction (e.g., lentiviral transduction) or otherwise delivered on a viral vector (e.g., fusogen-mediated delivery). In some embodiments, vectors that package a polynucleotide encoding an exogenous polynucleotide may be used to deliver the packaged polynucleotides to a cell or population of cells. These vectors may be of any kind, including DNA vectors, RNA vectors, plasmids, viral vectors and particles. In some embodiments, lipid nanoparticles can be used to deliver an exogenous polynucleotide to a cell. In some embodiments, viral vectors can be used to deliver an exogenous polynucleotide to a cell. Viral vector technology is well known and described in Sambrook et al. (2001, Molecular Cloning: A Laboratory Manual, Cold Spring Harbor Laboratory, New York). Viruses, which are useful as vectors include, but are not limited to lentiviral vectors, adenoviral vectors, adeno-associated viral (AAV) vectors, herpes simplex viral vectors, retroviral vectors, oncolytic viruses, and the like. In some embodiments, the introduction of the exogenous polynucleotide into the cell can be specific (targeted) or non-specific (e.g., non-targeted). In some embodiments, the introduction of the exogenous polynucleotide into the cell can result in integration or insertion into the genome in the cell. In other embodiments, the introduced exogenous polynucleotide may be non-integrating or episomal in the cell. A skilled artisan is familiar with methods of introducing nucleic acid transgenes into a cell, including any of the exemplary methods described herein, and can choose a suitable method.

### 1) Non-Targeted Delivery

In some embodiments, an exogenous polynucleotide is introduced into a cell (e.g., source cell) by any of a variety of non-targeted methods. In some embodiments, the exogenous polynucleotide is inserted into a random genomic locus of a host cell. As known to a person skilled in the art, viral vectors, including, for example, retroviral vectors and lentiviral vectors are commonly used to deliver genetic material into host cells and randomly insert the foreign or exogenous gene into the host cell genome to facilitate stable expression and replication of the gene. In some embodiments, the non-targeted introduction of the exogenous polynucleotide into the cell is under conditions for stable expression of the exogenous polynucleotide in the cell. In some embodiments, methods for introducing a nucleic acid for stable expression in a cell involves any method that results in stable integration of the nucleic acid into the genome of the cell, such that it may be propagated if the cell it has integrated into divides.

In some embodiments, the viral vector is a lentiviral vector. Lentiviral vectors are useful means for successful viral transduction as they permit stable expression of the gene contained within the delivered nucleic acid transcript. Lentiviral vectors express reverse transcriptase and integrase, two enzymes required for stable expression of the gene contained within the delivered nucleic acid transcript. Reverse transcriptase converts an RNA transcript into DNA, while integrase inserts and integrates the DNA into the genome of the target cell. Once the DNA has been integrated stably into the genome, it divides along with the host. The gene of interest contained within the integrated DNA may be expressed constitutively or it may be inducible. As part of the host cell genome, it may be subject to cellular regulation, including activation or repression, depending on a host of factors in the target cell.

Lentiviruses are subgroup of the Retroviridae family of viruses, named because reverse transcription of viral RNA genomes to DNA is required before integration into the host genome. As such, the most important features of lentiviral vehicles/particles are the integration of their genetic material into the genome of a target/host cell. Some examples of lentivirus include the Human Immunodeficiency Viruses: HIV-1 and HIV-2, the Simian Immunodeficiency Virus (SIV), feline immunodeficiency virus (FIV), bovine immunodeficiency virus (BIV), Jembrana Disease Virus (JDV), equine infectious anemia virus (EIAV), equine infectious anemia, virus, visna-maedi and caprine arthritis encephalitis virus (CAEV).

Typically, lentiviral particles making up the gene delivery vehicle are replication defective on their own (also referred to as “self-inactivating”). Lentiviruses are able to infect both dividing and non-dividing cells by virtue of the entry mechanism through the intact host nuclear envelope (Naldini L et al., Curr. Opin. Bioiecknol, 1998, 9: 457-463). Recombinant lentiviral vehicles/particles have been generated by multiply attenuating the HIV virulence genes, for example, the genes Env, Vif, Vpr, Vpu, Nef and Tat are deleted making the vector biologically safe. Correspondingly, lentiviral vehicles, for example, derived from HIV-1/HIV-2 can mediate the efficient delivery, integration and long-term expression of transgenes into non- dividing cells.

Lentiviral particles may be generated by co-expressing the virus packaging elements and the vector genome itself in a producer cell such as human HEK293T cells. These elements are usually provided in three (in second generation lentiviral systems) or four separate plasmids (in third generation lentiviral systems). The producer cells are co-transfected with plasmids that encode lentiviral components including the core (i.e. structural proteins) and enzymatic components of the virus, and the envelope protein(s) (referred to as the packaging systems), and a plasmid that encodes the genome including a foreign transgene, to be transferred to the target cell, the vehicle itself(also referred to as the transfer vector). In general, the plasmids or vectors are included in a producer cell line. The plasmids/vectors are introduced via transfection, transduction or infection into the producer cell line. Methods for transfection, transduction or infection are well known by those of skill in the art. As non-limiting example, the packaging and transfer constructs can be introduced into producer cell lines by calcium phosphate transfection, lipofection or electroporation, generally together with a dominant selectable marker, such as neomyocin (neo), dihydrofolate reductase (DHFR), glutamine synthetase or adenosine deaminase (ADA), followed by selection in the presence of the appropriate drug and isolation of clones.

The producer cell produces recombinant viral particles that contain the foreign gene, for example, the polynucleotides encoding the exogenous polynucleotide. The recombinant viral particles are recovered from the culture media and titrated by standard methods used by those of skill in the art. The recombinant lentiviral vehicles can be used to infect target cells, such source cells including any described in Section II.C.

Cells that can be used to produce high-titer lentiviral particles may include, but are not limited to, HEK293T cells, 293G cells, STAR cells (Relander et al., Mol Ther. 2005, 11: 452-459), FreeStyle™ 293 Expression System (ThermoFisher, Waltham, MA), and other HEK293T- based producer cell lines (e.g., Stewart et al., Hum Gene Ther. 2011, 2,2.(3):357-369; Lee et al, Biotechnol Bioeng, 2012, 10996); 1551-1560; Throm et al., Blood. 2009, 113(21); 5104-5110).

Additional elements provided in lentiviral particles may comprise retroviral LTR (long- terminal repeat) at either 5′ or 3′ terminus, a retroviral export element, optionally a lentiviral reverse response element (RRE), a promoter or active portion thereof, and a locus control region (LCR) or active portion thereof. Other elements include central polypurine tract (cPPT) sequence to improve transduction efficiency in non-dividing cells, Woodchuck Hepatitis Virus (WHP) Posttranscriptional Regulatory Element (WPRE) which enhances the expression of the transgene, and increases titer.

Methods for generating recombinant lentiviral particles are known to a skilled artisan, for example, U.S. Pat. Nos.: 8,846,385; 7,745,179; 7,629,153; 7,575,924; 7,179,903; and 6,808,905. Lentivirus vectors used may be selected from, but are not limited to pLVX, pLenti, pLenti6, pLJM1, FUGW, pWPXL, pWPI, pLenti CMV puro DEST, pLJM1-EGFP, pULTRA, pInducer2Q, pHIV-EGFP, pCW57.1, pTRPE, pELPS, pRRL, and pLionII, Any known lentiviral vehicles may also be used (See, U.S. Pat. Nos. 9,260,725; 9,068,199; 9,023,646; 8,900,858; 8,748,169; 8,709,799; 8,420,104; 8,329,462; 8,076,106; 6,013,516; and 5,994, 136: International Patent Publication NO.: WO2012079000).

In some embodiments, the exogenous polynucleotide is introduced into the cell under conditions for transient expression of the cell, such as by methods that result in episomal delivery of an exogenous polynucleotide.

In some embodiments, polynucleotides encoding the exogenous polynucleotide may be packaged into recombinant adeno-associated viral (rAAV) vectors. Such vectors or viral particles may be designed to utilize any of the known serotype capsids or combinations of serotype capsids. The serotype capsids may include capsids from any identified AAV serotypes and variants thereof, for example, AAV1, AAV2, AAV2G9, AAV3, AAV4, AAV4-4, AAV5, AAV6, AAV7, AAV8, AAV9, AAV10, AAV11, AAV12 and AAVrh10. In some embodiments, the AAV serotype may be or have a sequence as described in United States Publication No. US20030138772: Pulicherla et al. Molecular Therapy, 2011, 19(6); 1070-1078; U.S. Pat. Nos.: 6,156,303; 7,198.951; U.S. Patent Publication Nos.: US2015/0159173 and US2014/0359799; and International Patent Publication NOs.: WO1998/011244. WO2005/033321 and WO2014/14422.

AAV vectors include not only single stranded vectors but self-complementary AAV vectors (scAAVs). scAAV vectors contain DNA which anneals together to form double stranded vector genome. By skipping second strand synthesis, scAAVs allow for rapid expression in the cell. The rAAV vectors may be manufactured by standard methods in the art such as by triple transfection, in sf9 insect cells or in suspension cell cultures of human cells such as HEK293 cells.

In some embodiments, non-viral based methods may be used. For instance, in some aspects, vectors comprising the polynucleotides may be transferred to cells by non-viral methods by physical methods such as needles, electroporation, sonoporation, hyrdoporation; chemical carriers such as inorganic particles (e.g., calcium phosphate, silica, gold) and/or chemical methods. In other aspects, synthetic or natural biodegradable agents may be used for delivery such as cationic lipids, lipid nano emulsions, nanoparticles, peptide based vectors, or polymer based vectors.

In some embodiments, an mRNA based method may be used.

### 2) Targeted Delivery

The exogenous polynucleotide can be inserted into any suitable target genomic loci of the cell. In some embodiments, the exogenous polynucleotide is introduced into the cell by targeted integration into a target loci. In some embodiments, targeted integration can be achieved by gene editing using one or more nucleases and/or nickases and a donor template in a process involving homology-dependent or homology-independent recombination.

A number of gene editing methods can be used to insert an exogenous polynucleotide into the specific genomic locus of choice, including for example homology-directed repair (HOR), homology-mediated end-joining (HMEJ), homology-independent targeted integration (HITI), obligate ligation-gated recombination (ObliGaRe), or precise integration into target chromosome (PITCh).

In some embodiments, the nucleases create specific double-strand breaks (DSBs) at desired locations (e.g., target sites) in the genome, and harness the cell's endogenous mechanisms to repair the induced break. The nickases create specific single-strand breaks at desired locations in the genome. In one non-limiting example, two nickases can be used to create two single-strand breaks on opposite strands of a target DNA, thereby generating a blunt or a sticky end. Any suitable nuclease can be introduced into a cell to induce genome editing of a target DNA sequence including, but not limited to, CRISPR-associated protein (Cas) nucleases, zinc finger nucleases (ZFNs), transcription activator-like effector nucleases (TALENs), meganucleases, other endo- or exo-nucleases, variants thereof, fragments thereof, and combinations thereof. In some embodiments, when a nuclease or a nickase is introduced with a donor template containing an exogenous polynucleotide sequence (also called a transgene) flanked by homology sequences (e.g., homology arms) that are homologous to sequences at or near the endogenous genomic target locus, e.g., a safe harbor locus, DNA damage repair pathways can result in integration of the transgene sequence at the target site in the cell. This can occur by a homology-dependent process. In some embodiments, the donor template is a circular double-stranded plasmid DNA, single-stranded donor oligonucleotide (ssODN), linear double-stranded polymerase chain reaction (PCR) fragments, or the homologous sequences of the intact sister chromatid. Depending on the form of the donor template, the homology-mediated gene insertion and replacement can be carried out via specific DNA repair pathways such as homology-directed repair (HDR), synthesis-dependent strand annealing (SDSA), microhomology-mediated end joining (MMEJ), and homology-mediated end joining (HMEJ) pathways.

For instance, DNA repair mechanisms can be induced by a nuclease after (i) two SSBs, where there is a SSB on each strand, thereby inducing single strand overhangs; or (ii) a DSB occurring at the same cleavage site on both strands, thereby inducing a blunt end break. Upon cleavage by one of these agents, the target locus with the SSBs or the DSB undergoes one of two major pathways for DNA damage repair: (1) the error-prone non-homologous end joining (NHEJ), or (2) the high-fidelity homology-directed repair (HDR) pathway. In some embodiments, a donor template (e.g., circular plasmid DNA or a linear DNA fragment, such as a ssODN) introduced into cells in which there are SSBs or a DSB can result in HDR and integration of the donor template into the target locus. In general, in the absence of a donor template, the NHEJ process re-ligates the ends of the cleaved DNA strands, which frequently results in nucleotide deletions and insertions at the cleavage site.

In some embodiments, site-directed insertion of the exogenous polynucleotide into a cell may be achieved through HDR-based approaches. HDR is a mechanism for cells to repair double-strand breaks (DSBs) in DNA and can be utilized to modify genomes in many organisms using various gene editing systems, including clustered regularly interspaced short palindromic repeat (CRISPR)/Cas systems, zinc finger nucleases (ZFNs), transcription activator-like effector nucleases (TALENs), meganucleases, and transposases.

In some embodiments, the targeted integration is carried by introducing one or more sequence-specific or targeted nucleases, including DNA-binding targeted nucleases and gene editing nucleases such as zinc finger nucleases (ZFN) and transcription activator-like effector nucleases (TALENs), and RNA-guided nucleases such as a CRISPR-associated nuclease (Cas) system, specifically designed to be targeted to at least one target site(s) sequence of a target gene. Exemplary ZFNs, TALEs, and TALENs are described in, e.g., Lloyd et al., Frontiers in Immunology, 4(221); 1-7 (2013). In embodiments, targeted genetic disruption at or near the target site is carried out using clustered regularly interspaced short palindromic repeats (CRISPR) and CRISPR-associated (Cas) proteins. See Sander and Joung, (2014) Nature Biotechnology, 32(4); 347-355.

Any of the systems for gene disruption described in Section II. A.1 can be used and, when also introduced with an appropriate donor template having with an exogenous polynucleotide, e.g., transgene sequences, can result in targeted integration of the exogenous polynucleotide at or near the target site of the genetic disruption. In embodiments, the genetic disruption is mediated using a CRISPR/Cas system containing one or more guide RNAs (gRNA) and a Cas protein. Exemplary Cas proteins and gRNA are described in Section II.A above, any of which can be used in HDR mediated integration of an exogenous polynucleotide into a target locus to which the Crispr/Cas system is specific for. It is within the level of a skilled artisan to choose an appropriate Cas nuclease and gRNA, such as depending on the particular target locus and target site for cleavage and integration of the exogenous polynucleotide by HDR. Further, depending on the target locus a skilled artisan can readily prepare an appropriate donor template, such as described further below.

In some embodiments, the DNA editing system is an RNA-guided CRISPR/Cas system (such as RNA-based CRISPR/Cas system), wherein the CRISPR/Cas system is capable of creating a double-strand break in the target locus (e.g., safe harbor locus) to induce insertion of the transgene into the target locus. In some embodiments, the nuclease system is a CRISPR/Cas9 system. In some embodiments, the CRISPR/Cas9 system comprises a plasmid-based Cas9. In some embodiments, the CRISPR/Cas9 system comprises a RNA-based Cas9. In some embodiments, the CRISPR/Cas9 system comprises a Cas9 mRNA and gRNA. In some embodiments, the CRISPR/Cas9 system comprises a protein/RNA complex, or a plasmid/RNA complex, or a protein/plasmid complex. In some embodiments, there are provided methods for generating engineered cells, which comprises introducing into a source cell (e.g., a primary cell or a pluripotent stem cell, e.g., iPSC) a donor template containing a transgene or exogenous polynucleotide sequence and a DNA nuclease system including a DNA nuclease system (e.g., Cas9) and a locus-specific gRNA. In some embodiments, the Cas9 is introduced as an mRNA. In some embodiments, the Cas9 is introduced as a ribonucleoprotein complex with the gRNA.

Generally, the donor template to be inserted would comprise at least the transgene cassette containing the exogenous polynucleotide of interest (e.g., the tolerogenic factor or CAR) and would optionally also include the promoter. In certain of these embodiments, the transgene cassette containing the exogenous polynucleotide and/or promoter to be inserted would be flanked in the donor template by homology arms with sequences homologous to sequences immediately upstream and downstream of the target cleavage site, i.e., left homology arm (LHA) and right homology arm (RHA). Typically, the homology arms of the donor template are specifically designed for the target genomic locus to serve as template for HDR. The length of each homology arm is generally dependent on the size of the insert being introduced, with larger insertions requiring longer homology arms.

In some embodiments, a donor template (e.g., a recombinant donor repair template) comprises: (i) a transgene cassette comprising an exogenous polynucleotide sequence (for example, a transgene operably linked to a promoter, for example, a heterologous promoter); and (ii) two homology arms that flank the transgene cassette and are homologous to portions of a target locus (e.g., safe harbor locus) at either side of a DNA nuclease (e.g., Cas nuclease, such as Cas9 or Cas12) cleavage site. The donor template can further comprise a selectable marker, a detectable marker, and/or a purification marker.

In some embodiments, the homology arms are the same length. In other embodiments, the homology arms are different lengths. The homology arms can be at least about 10 base pairs (bp), e.g., at least about 10 bp, 15 bp, 20 bp, 25 bp, 30 bp, 35 bp, 45 bp, 55 bp, 65 bp, 75 bp, 85 bp, 95 bp, 100 bp, 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1000 bp, 1.1 kilobases (kb), 1.2 kb, 1.3 kb, 1.4 kb, 1.5 kb, 1.6 kb, 1.7 kb, 1.8 kb, 1.9 kb, 2.0 kb, 2, 1 kb, 2,2 kb, 2.3 kb, 2,4 kb, 2,5 kb, 2,6 kb, 2.7 kb, 2.8 kb, 2.9 kb, 3.0 kb, 3.1 kb, 3.2 kb, 3.3 kb, 3.4 kb, 3.5 kb, 3.6 kb, 3.7 kb, 3.8 kb, 3.9 kb, 4.0 kb, or longer. The homology arms can be about 10 bp to about 4 kb, e.g., about 10 bp to about 20 bp, about 10 bp to about 50 bp, about 10 bp to about 100 bp, about 10 bp to about 200 bp, about 10 bp to about 500 bp, about 10 bp to about 1 kb, about 10 bp to about 2 kb, about 10 bp to about 4 kb, about 100 bp to about 200 bp, about 100 bp to about 500 bp, about 100 bp to about 1 kb, about 100 bp to about 2 kb, about 100 bp to about 4 kb, about 500 bp to about 1 kb, about 500 bp to about 2 kb, about 500 bp to about 4 kb, about 1 kb to about 2 kb, about 1 kb to about 2 kb, about 1 kb to about 4 kb, or about 2 kb to about 4 kb.

In some embodiments, the donor template can be cloned into an expression vector. Conventional viral and non-viral based expression vectors known to those of ordinary skill in the art can be used.

In some embodiments, the target locus targeted for integration may be any locus in which it would be acceptable or desired to target integration of an exogenous polynucleotide or transgene. Non-limiting examples of a target locus include, but are not limited to, a CXCR4 gene, an albumin gene, a SHS231 locus, an F3 gene (also known as CD142), a MICA gene, a MICB gene, a LRP1 gene (also known as CD91), a HMGB1 gene, an ABO gene, a RHD gene, a FUT1 gene, a KDM5D gene (also known as HY), a B2M gene, a CIITA gene, a TRAC gene, a TRBC gene, a CCR5 gene, a F3 (i.e., CD142) gene, a MICA gene, a MICB gene, a LRP1 gene, a HMGB1 gene, an ABO gene, a RHD gene, a FUT1 gene, a KDM5D (i.e., HY) gene, a PDGFRa gene, a OLIG2 gene, and/or a GFAP gene. In some embodiments, the exogenous polynucleotide can be inserted in a suitable region of the target locus (e.g., safe harbor locus), including, for example, an intron, an exon, and/or gene coding region (also known as a Coding Sequence, or “CDS”). In some embodiments, the insertion occurs in one allele of the target genomic locus. In some embodiments, the insertion occurs in both alleles of the target genomic locus. In either of these embodiments, the orientation of the transgene inserted into the target genomic locus can be either the same or the reverse of the direction of the gene in that locus.

In some embodiments, the exogenous polynucleotide is interested into an intron, exon, or coding sequence region of the safe harbor gene locus. In some embodiments, the exogenous polynucleotide is inserted into an endogenous gene wherein the insertion causes silencing or reduced expression of the endogenous gene. Exemplary genomic loci for insertion of an exogenous polynucleotide are depicted in Table 2B.

TABLE 2B
Exemplary genomic loci for insertion of exogenous polynucleotides

| | | | | Target region for | |
|---|---|---|---|---|---|
| Number | species | name | Ensembl ID | cleavage | Also known as |
| 1 | human | B2M | ENSG00000166710 | CDS | |
| 2 | human | CIITA | ENSG00000179583 | CDS | |
| 3 | human | TRAC | ENSG00000277734 | CDS | |
| 4 | human | PPP1R12C | ENSG00000125503 | Intron 1 and 2 | AAVS1 |
| 5 | human | CLYBL | ENSG00000125246 | Intron 2 | |
| 6 | human | CCR5 | ENSG00000160791 | Exons 1-3, | |
| | | | | introns 1-2, and | |
| | | | | CDS | |
| 7 | human | THUMPD3-AS1 | ENSG00000206573 | Intron 1 | ROSA26 |
| 8 | human | Ch− 4: 58,976,613 | | 500 bp window | SHS231 |
| 9 | human | F3 | ENSG00000117525 | CDS | CD142 |
| 10 | human | MICA | ENSG00000204520 | CDS | |
| 11 | human | MICB | ENSG00000204516 | CDS | |
| 12 | human | LRP1 | ENSG00000123384 | CDS | |
| 13 | human | HMGB1 | ENSG00000189403 | CDS | |
| 14 | human | ABO | ENSG00000175164 | CDS | |
| 15 | human | RHD | ENSG00000187010 | CDS | |
| 16 | human | FUT1 | ENSG00000174951 | CDS | |
| 17 | human | KDM5D | ENSG00000012817 | CDS | HY |

In some embodiments, the target locus is a safe harbor locus. In some embodiments, a safe harbor locus is a genomic location that allows for stable expression of integrated DNA with minimal impact on nearby or adjacent endogenous genes, regulatory element and the like. In some cases, a safe harbor gene enables sustainable gene expression and can be targeted by engineered nuclease for gene modification in various cell types including primary cells and pluripotent stem cells, including derivatives thereof, and differentiated cells thereof. Non-limiting examples of a safe harbor locus include, but are not limited to, a CCR5 gene locus, a PPP112C (also known as AAVS1) gene locus, a CLYBL gene locus, and/or a Rosa gene locus (e.g., ROSA26 gene locus). In some embodiments, the safe harbor locus is selected from the group consisting of the AAVS1 locus, the CCR5 locus, and the CLYBL locus. In some cases SHS231 can be targeted as a safe harbor locus in many cell types. In some cases, certain loci can function as a safe harbor locus in certain cell types. For instance, PDGFRa is a safe harbor for glial progenitor cells (GPCs), OLIG2 is a safe harbor locus for oligodendrocytes, and GFAP is a safe harbor locus for astrocytes. It is within the level of a skilled artisan to choose an appropriate safe harbor locus depending on the particular engineered cell type. In some cases, more than one safe harbor gene can be targeted, thereby introducing more than one transgene into the genetically modified cell.

In some embodiments, there are provided methods for generating engineered cells, which comprises introducing into a source cell (e.g., a primary cell or a pluripotent stem cell, e.g., iPSC) a donor template containing a transgene or exogenous polynucleotide sequence and a DNA nuclease system including a DNA nuclease system (e.g., Cas9) and a locus-specific gRNA that comprise complementary portions (e.g., gRNA targeting sequence) specific to a CCR5 gene locus, a PPP1R12C (also known as AAVS1) gene locus, a CLYBL gene locus, and/or a Rosa gene locus (e.g., ROSA26 gene locus). In some embodiments, the genomic locus targeted by the gRNAs is located within 4000 bp, within 3500 bp, within 3000 bp, within 2500 bp, within 2000 bp, within 1500 bp, within 1000 bp, or within 500 bp of any of the loci as described.

In some embodiments, the gRNAs used herein for HDR-mediated insertion of a transgene comprise a complementary portion (e.g., gRNA targeting sequence) that recognizes a target sequence in AAVS1. In certain of these embodiments, the target sequence is located in intron 1 of AAVS 1. AAVS1 is located at Chromosome 19: 55,090,918-55,117,637 reverse strand, and AAVS1 intron 1 (based on transcript ENSG00000125503) is located at Chromosome 19: 55,117,222-55,112,796 reverse strand. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 19: 55, 117,222-55, 112,796. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 19: 55,115,674. In certain embodiments, the gRNA is configured to produce a cut site at Chromosome 19: 55, 115,674, or at a position within 5, 10, 15, 20, 30, 40 or 50 nucleotides of Chromosome 19: 55, 115,674. In certain embodiments, the gRNA s GET000046, also known as “sgAAVS1-1.” described in Li et al., Nat. Methods 16:866-869 (2019). This gRNA comprises a complementary portion (e.g., gRNA targeting sequence) having the nucleic acid sequence set forth in SEQ ID NO: 36 (shown in Table 17) and targets intron 1 of AAVS1 (also known as PPP1R12C).

In some embodiments, the gRNAs used herein for HDR-mediated insertion of a transgene comprise a complementary portion (e.g., gRNA targeting sequence) that recognizes a target sequence in CLYBL. In certain of these embodiments, the target sequence is located in intron 2 of CLYBL. CLYBL is located at Chromosome 13: 99,606,669-99,897, 134 forward strand, and CLYBL intron 2 (based on transcript ENST00000376355.7) is located at Chromosome 13: 99,773,011-99,858,860 forward strand. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 13: 99,773,011-99,858.860. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 13: 99,822,980. In certain embodiments, the gRNA is configured to produce a cut site at Chromosome 13: 99,822,980, or at a position within 5, 0, 15, 20, 30, 40 or 50 nucleotides of Chromosome 13: 99,822,980. In certain embodiments, the gRNA is GET000047, which comprises a complementary portion (e.g., gRNA targeting sequence) having the nucleic acid sequence set forth in SEQ ID NO: 36 (shown in Table 17) and targets intron 2 of CLYBL. The target site is similar to the target site of the TALENs as described in Cerbini et al., PLoS One, 10(1):e0116032 (2015).

In some embodiments, the gRNAs used herein for HDR-mediated insertion of a transgene comprise a complementary portion (e.g., gRNA targeting sequence) that recognizes a target sequence in CCR5. In certain of these embodiments, the target sequence is located in exon 3 of CCR5. CCR5 is located at Chromosome 3: 46,370,854-46,376,206 forward strand, and CCR5 exon 3 (based on transcript ENST00000292303.4) is located at Chromosome 3: 46,372,892-46,376,206 forward strand. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 3: 46,372,892-46,376,206. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 3: 46,373,180. In certain embodiments, the gRNA is configured to produce a cut site at Chromosome 3: 46,373,180, or at a position within 5, 10, 15, 20, 30, 40, or 50 nucleotides of Chromosome 3: 46,373,180. In certain embodiments the gRNA is GET000048, also known as “crCCR5_D,” described in Mandal et al., Cell Stem Cell 15:643-652 (2014). This gRNA comprises a complementary portion having the nucleic acid sequence set forth in SEQ ID NO: 37 (shown in Table 17) and targets exon 3 of CCR5 (alternatively annotated as exon 2 in the Ensembl genome database). See Gomez-Ospina et al., Nat. Comm. 10(1):4045 (2019).

Table 17 sets forth exemplary gRNA targeting sequences. In some embodiments, the gRNA targeting sequence may contain one or more thymines in the complementary portion sequences set forth in Table 17 are substituted with uracil. It will be understood by one of ordinary skill in the art that uracil and thymine can both be represented by ‘t’, instead of ‘u’ for uracil and ‘t’ for thymine: in the context of a ribonucleic acid, it will be understood that ‘t’ is used to represent uracil unless otherwise indicated.

TABLE 17
Exemplary gRNA targeting sequences for CCR5

| | | SEQ |
|---|---|---|
| Description | Nucleic Acid Sequence | ID NO: |
| GET000046 | (5′→3′)accccacagtggggccacta | 35 |
| guide | | |
| GET000047 | (5′→3′)tgttggaaggatgaggaaat | 36 |
| guide | | |
| GET000048 | (5′→3′)tcactatgctgccgcccagt | 37 |
| guide | | |

In some embodiments, the target locus is a locus that is desired to be knocked out in the cells. In such embodiments, such a target locus is any target locus whose disruption or elimination is desired in the cell, such as to modulate a phenotype or function of the cell. For instance, any of the gene modifications described in Section II.A to reduce expression of a target gene may be a desired target locus for targeted integration of an exogenous polynucleotide, in which the genetic disruption or knockout of a target gene and overexpression by targeted insertion of an exogenous polynucleotide may be achieved at the same target site or locus in the cell. For instance, the HDR process may be used to result in a genetic disruption to eliminate or reduce expression of (e.g., knock out) any target gene set forth in Table 1 while also integrating (e.g., knocking in) an exogenous polynucleotide into the target gene by using a donor template with flanking homology arms that are homologous to nucleic acid sequences at or near the target site of the genetic disruption.

In some embodiments, there are provided methods for generating engineered cells, which comprises introducing into a source cell (e.g., a primary cell or a pluripotent stem cell, e.g., iPSC) a donor template containing a transgene or exogenous polynucleotide sequence and a DNA nuclease system including a DNA nuclease system (e.g., Cas9) and a locus-specific gRNA that comprise complementary portions specific to the B2M locus, the CIITA locus, the TRAC locus, the TRBC locus. In some embodiments, the genomic locus targeted by the gRNAs is located within 4000 bp, within 3500 bp, within 3000 bp, within 2500 bp, within 2000 bp, within 1500 bp, within 1000 bp, or within 500 bp of any of the loci as described.

In embodiments, the target locus is B2M. In some embodiments, the engineered cell comprises a genetic modification targeting the B2M gene. In some embodiments, the genetic modification targeting the B2M gene is by using a targeted nuclease system that comprises a Cas protein or a polynucleotide encoding a Cas protein, and at least one guide ribonucleic acid sequence for specifically targeting the B2M gene. In some embodiments, the at least one guide ribonucleic acid (gRNA) sequence for specifically targeting the B2M gene is selected from the group consisting of SEQ ID NOS:81240-85644 of Appendix 2 or Table 15 of WO2016/183041, the disclosure of which is herein incorporated by reference in its entirety. In some embodiments, an exogenous polynucleotide is integrated into the disrupted B2M locus by HDR by introducing a donor template containing the exogenous polynucleotide sequence with flanking homology arms homologous to sequences adjacent to the target site targeted by the gRNA.

In embodiments, the target locus is CIITA. In some embodiments, the engineered cell comprises a genetic modification targeting the CIITA gene. In some embodiments, the genetic modification targeting the CIITA gene is by a targeted nuclease system that comprises a Cas protein or a polynucleotide encoding a Cas protein, and at least one guide ribonucleic acid sequence for specifically targeting the CIITA gene. In some embodiments, the at least one guide ribonucleic acid sequence for specifically targeting the CIITA gene is selected from the group consisting of SEQ ID NOS:5184-36352 of Appendix 1 or Table 12 of WO2016183041, the disclosure of which is herein incorporated by reference in its entirety. In some embodiments, an exogenous polynucleotide is integrated into the disrupted CIITA locus by HDR by introducing a donor template containing the exogenous polynucleotide sequence with flanking homology arms homologous to sequences adjacent to the target site targeted by the gRNA.

In some embodiments, the cell is a T cell and expression of the endogenous TRAC or TRBC locus is reduced or eliminated in the cell by gene editing methods. For instance, the HDR process may be used to result in a genetic disruption to eliminate or reduce expression of(e.g., knock out) the TRAC or a TRBC gene while also integrating (e.g., knocking in) an exogenous polynucleotide into the same locus by using a donor template with flanking homology arms that are homologous to nucleic acid sequences at or near the target site of the genetic disruption. Exemplary gRNA sequences useful for CRISPR/Cas-based targeting of genes described herein are provided in Table 2C. The sequences can be found in US20160348073, the disclosure including the Sequence Listing is incorporated herein by reference in its entirety.

TABLE 2C
Exemplary gRNA targeting sequences useful for targeting genes

| Gene Name | SEQ ID NO in US 20160348073 |
|---|---|
| TRAC | SEQ ID NOS: 532-609 and 9102-9797 |
| TRB (also | SEQ ID NOS: 610-765 and 9798-10532 |
| TCRB, and | |
| TRBC) | |

In some embodiments, the engineered cell comprises a genetic modification targeting the TRAC gene. In some embodiments, the genetic modification targeting the TRAC gene is by a targeted nuclease system that comprises a Cas protein or a polynucleotide encoding a Cas protein, and at least one guide ribonucleic acid sequence for specifically targeting the TRAC gene. In some embodiments, the at least one guide ribonucleic acid sequence (e.g., gRNA targeting sequence) for specifically targeting the TRAC gene is selected from the group consisting of SEQ ID NOS: SEQ ID NOS: 532-609 and 9102-9797 of US20160348073, the disclosure of which is herein incorporated by reference in its entirety. In some embodiments, an exogenous polynucleotide is integrated into the disrupted TRAC locus by HDR by introducing a donor template containing the exogenous polynucleotide sequence with flanking homology arms homologous to sequences adjacent to the target site targeted by the gRNA.

In some embodiments, the engineered cell comprises a genetic modification targeting the TRBC gene. In some embodiments, the genetic modification targeting the TRBC gene is by a targeted nuclease system that comprises a Cas protein or a polynucleotide encoding a Cas protein, and at least one guide ribonucleic acid sequence for specifically targeting the TRBC gene. In some embodiments, the at least one guide ribonucleic acid sequence (e.g., gRNA targeting sequence) for specifically targeting the TRBC gene is selected from the group consisting of SEQ ID NOS: SEQ ID NOS:610-765 and 9798-10532 of US20160348073, the disclosure of which is herein incorporated by reference in its entirety. In some embodiments, an exogenous polynucleotide is integrated into the disrupted TRBC locus by HDR by introducing a donor template containing the exogenous polynucleotide sequence with flanking homology arms homologous to sequences adjacent to the target site targeted by the gRNA.

In some embodiments, it is within the level of a skilled artisan to identify new loci and/or gRNA sequences for use in HDR-mediated integration approaches as described. For example, for CRISPR/Cas systems, when an existing gRNA for a particular locus (e.g., within a target gene, e.g., set forth in Table 1) is known, an “inch worming” approach can be used to identify additional loci for targeted insertion of transgenes by scanning the flanking regions on either side of the locus for PAM sequences, which usually occurs about every 100 base pairs (bp) across the genome. The PAM sequence will depend on the particular Cas nuclease used because different nucleases usually have different corresponding PAM sequences. The flanking regions on either side of the locus can be between about 500 to 4000 bp long, for example, about 500 bp, about 1000 bp, about 1500 bp, about 2000 bp, about 2500 bp, about 3000 bp, about 3500 bp, or about 4000 bp long. When a PAM sequence is identified within the search range, a new guide can be designed according to the sequence of that locus for use in genetic disruption methods. Although the CRISPR/Cas system is described as illustrative, any HDR-mediated approaches as described can be used in this method of identifying new loci, including those using ZFNs. TALENS, meganucleases and transposases.

In some embodiments, the exogenous polynucleotide encodes an exogenous CD47 polypeptide (e.g., a human CD47 polypeptide) and the exogenous polypeptide is inserted into a safe harbor gene loci or a safe harbor site as disclosed herein or a genomic locus that causes silencing or reduced expression of the endogenous gene. In some embodiments, the exogenous polynucleotide encoding CD47 is inserted in a CCR5 gene locus, a PPP1R12C (also known as AAVS1) gene locus, a CLYBL gene locus, and/or a Rosa gene locus (e.g., ROSA26 gene locus). In some embodiments, the polynucleotide is inserted in a B2M, CIITA, TRAC, TRBC, PD1 or CTLA4 gene locus.

### C. Cells

In some embodiments, the present disclosure provides a cell (e.g., stem cell, induced pluripotent stem cell, differentiated cell derived or produced from such stem cell, hematopoietic stem cell, or primary cell), or population thereof, that has been engineered (or modified) in which the genome of the cell has been modified such that expression of one or more genes as described herein is reduced or deleted (e.g., gene regulating expression of one or more MHC class I molecules or one or more MHC class II molecules) or in which a gene or polynucleotide is overexpressed or increased in expression (e.g., polynucleotide encoding tolerogenic factor, such as CD47).

Embodiments relating to stem cells disclosed herein may be taken to also disclose the corresponding embodiment where the cell is a cell derived from such a stem cell.

In some embodiments, the engineered cell that includes the exogenous polynucleotide is a beta islet cell and includes a first exogenous polynucleotide that encodes a CD47 polypeptide. In some embodiments, the engineered beta islet cell further comprises one or more additional exogenous polynucleotides that encode one or more complement inhibitors or other tolerogenic polypeptides described herein. In some embodiments, the first exogenous polynucleotide and the one or more additional exogenous polynucleotide are inserted into the same genomic locus. In some embodiments, the first exogenous polynucleotide and the one or more additional exogenous polynucleotide are inserted into different genomic loci. In exemplary embodiments, the engineered (e.g., hypoimmunogenic) cell is a primary beta islet cell or a beta islet cell derived from an engineered (e.g., hypoimmunogenic) pluripotent cell (e.g., an iPSC).

In some embodiments, the engineered cell that includes the exogenous polynucleotide is a hepatocyte and includes a first exogenous polynucleotide that encodes a CD47 polypeptide. In some embodiments, the engineered hepatocyte further comprises one or more additional exogenous polynucleotides that encode one or more complement inhibitors or other tolerogenic polypeptides described herein. In some embodiments, the first exogenous polynucleotide and the one or more additional exogenous polynucleotide are inserted into the same genomic locus. In some embodiments, the first exogenous polynucleotide and the one or more additional exogenous polynucleotide are inserted into different genomic loci. In exemplary embodiments, the engineered (e.g., hypoimmunogenic) cell is a primary hepatocyte or a hepatocyte cell derived from an engineered (e.g., hypoimmunogenic) pluripotent cell (e.g., an iPSC).

In some embodiments, the cells that are engineered or modified as provided herein are pluripotent stems cells or are cells differentiated from pluripotent stem cells. In some embodiments, the cells that are engineered or modified as provided herein are primary cells.

The cell may be a vertebrate cell, for example, a mammalian cell, such as a human cell or a mouse cell. The cell may also be a vertebrate stem cell, for example, a mammalian stem cell, such as a human stem cell (or a cell derived from such a stem cell), or a mouse stem cell (or a cell derived from such a stem cell). In embodiments, the cell or stem cell or a cell derived from such a stem cell is amenable to modification.

In embodiments, the cell or stem cell, or a cell derived from such a stem cell, has or is believed to have therapeutic value, such that the cell or stem cell or a cell derived or differentiated from such stem cell may be used to treat a disease, disorder, defect or injury in a subject in need of treatment for same.

In some embodiments, the cell is a stem cell or progenitor cell (e.g., iPSC, embryonic stem cell, hematopoietic stem cell, mesenchymal stem cell, endothelial stem cell, epithelial stem cell, adipose stem or progenitor cells, germline stem cells, lung stem or progenitor cells, mammary stem cells, olfactory adult stem cells, hair follicle stem cells, multipotent stem cells, amniotic stem cells, cord blood stem cells, or neural stem or progenitor cells). In some embodiments, the stem cells are adult stem cells (e.g., somatic stem cells or tissue specific stem cells). In some embodiments, the stem or progenitor cell is capable of being differentiated (e.g., the stem cell is totipotent, pluripotent, or multipotent). In some embodiments, the cell is isolated from embryonic or neonatal tissue. In some embodiments, the cell is a fibroblast, monocytic precursor, B cell, exocrine cell, pancreatic progenitor, endocrine progenitor, hepatoblast, myoblast, preadipocyte, progenitor cell, hepatocyte, chondrocyte, smooth muscle cell, K562 human erythroid leukemia cell line, bone cell, synovial cell, tendon cell, ligament cell, meniscus cell, adipose cell, dendritic cells, or natural killer cell. In some embodiments, the cell is manipulated (e.g., converted or differentiated) into a muscle cell, erythroid-megakaryocytic cell, eosinophil, iPS cell, macrophage, T cell, islet beta-cell, neuron, cardiomyocyte, blood cell, endocrine progenitor, exocrine progenitor, ductal cell, acinar cell, alpha cell, beta cell, delta cell, PP cell, hepatocyte, cholangiocyte, or brown adipocyte. In some embodiments, the cell is a muscle cell (e.g., skeletal, smooth, or cardiac muscle cell), erythroid-megakaryocytic cell, eosinophil, iPS cell, macrophage, T cell, islet beta-cell, neuron, cardiomyocyte, blood cell (e.g., red blood cell, white blood cell, or platelet), endocrine progenitor, exocrine progenitor, ductal cell, acinar cell, alpha cell, beta cell, delta cell, PP cell, hepatocyte, cholangiocyte, or white or brown adipocyte. In some embodiments, the cell is a hormone-secreting cell (e.g., a cell that secretes insulin, oxytocin, endorphin, vasopressin, serotonin, somatostatin, gastrin, secretin, glucagon, thyroid hormone, bombesin, cholecystokinin, testosterone, estrogen, or progesterone, renin, ghrelin, amylin, or pancreatic polypeptide), an epidermal keratinocyte, an epithelial cell (e.g., an exocrine secretory epithelial cell, a thyroid epithelial cell, a keratinizing epithelial cell, a gall bladder epithelial cell, or a surface epithelial cell of the cornea, tongue, oral cavity, esophagus, anal canal, distal urethra, or vagina), a kidney cell, a germ cell, a skeletal joint synovium cell, a periosteum cell, a bone cell (e.g., osteoclast or osteoblast), a perichondrium cell (e.g., a chondroblast or chondrocyte ), a cartilage cell (e.g., chondrocyte), a fibroblast, an endothelial cell, a pericardium cell, a meningeal cell, a keratinocyte precursor cell, a keratinocyte stem cell, a pericyte, a glial cell, an ependymal cell, a cell isolated from an amniotic or placental membrane, or a serosal cell (e.g., a serosal cell lining body cavities).

In some embodiments, the cell is a somatic cell. In some embodiments, the cells are derived from skin or other organs, e.g., heart, brain or spinal cord, liver, lung, kidney, pancreas, bladder, bone marrow, spleen, intestine, or stomach. The cells can be from humans or other mammals (e.g., rodent, non-human primate, bovine, or porcine cells).

In some embodiments, the cell is a T cell, NK cell, beta islet cells, endothelial cell, epithelial cell such as RPE, thyroid, skin, or hepatocytes. In some embodiments, the cell is an iPSC-derived cell that has been differentiated from an engineered iPSC. In some embodiments, the cell is an engineered cell that has been modified from a primary cell. In some embodiments, the cell comprises increased expression of one or more tolerogenic factors. In some embodiments, the one or more tolerogenic factor is CD47.

In some embodiments, the cell comprises an exogenous polynucleotide encoding CD47. In some embodiments, the cell comprises overexpression or increased expression of one or more complement inhibitor.

In some embodiments, the cell is an iPSC-derived T cell that is engineered to contain modifications (e.g., genetic modifications) described herein. In some embodiments, the cell is a primary T cell that is engineered to contain modifications (e.g., genetic modifications) described herein. In some embodiments, the cell comprises overexpression or increased expression of one or more complement inhibitor. In some embodiments, the T cell can be engineered with a chimeric antigen receptor (CAR), including any as described herein. In some embodiments, the engineered (e.g., hypoimmunogenic) T cell can be used to treat a variety of indications with allogenic cell therapy, including any as described herein, e.g., Section IV. In some embodiments, the engineered (e.g., hypoimmunogenic) T cell can be used to treat cancer.

In some embodiments, the cell is an iPSC-derived NK cell that is engineered to contain modifications (e.g., genetic modifications) described herein. In some embodiments, the cell is a primary NK cell that is engineered to contain modifications (e.g., genetic modifications) described herein. In some embodiments, the cell comprises overexpression or increased expression of one or more complement inhibitor. In some embodiments, the NK cell can be engineered with a chimeric antigen receptor (CAR), including any as described herein. In some embodiments, the engineered (e.g., hypoimmunogenic) NK cell can be used to treat a variety of indications with allogenic cell therapy, including any as described herein, e.g., Section V. In some embodiments, the engineered (e.g., hypoimmunogenic) NK cell can be used to treat cancer.

In some embodiments, the cell is an iPSC-derived beta-islet cell that is engineered to contain modifications (e.g., genetic modifications) described herein. In some embodiments, the cell is a primary beta-islet cell that is engineered to contain modifications (e.g., genetic modifications) described herein. In some embodiments, the cell comprises overexpression or increased expression of one or more complement inhibitor. In some embodiments, the engineered (e.g., hypoimmunogenic) beta-islet cell can be used to treat a variety of indications with allogenic cell therapy, including any as described herein. e.g., Section V. In some embodiments, the engineered (e.g., hypoimmunogenic) beta-islet cell can be used to treat diabetes, such as type I diabetes.

In some embodiments, the cell is an iPSC-derived endothelial cells that is engineered to contain modifications (e.g., genetic modifications) described herein. In some embodiments, the cell is a primary endothelial cell that is engineered to contain modifications (e.g., genetic modifications) described herein. In some embodiments, the engineered (e.g., hypoimmunogenic) endothelial cell can be used to treat a variety of indications with allogenic cell therapy, including any as described herein, e.g., Section V. In some embodiments, the engineered (e.g., hypoimmunogenic) endothelial cell can be used to treat vascularization or ocular diseases.

In some embodiments, the cell is an iPSC-derived epithelial cell that is engineered to contain modifications (e.g., genetic modifications) described herein. In some embodiments, the cell is a primary epithelial cell that is engineered to contain modifications (e.g., genetic modifications) described herein. In some embodiments, the epithelial cell is an RPE. In some embodiments, the epithelial cell is a thyroid cell. In some embodiments, the epithelial cell is a skin cell. In some embodiments, the engineered (e.g., hypoimmunogenic) epithelial cell can be used to treat a variety of indications with allogenic cell therapy, including any as described herein, e.g., Section V. In some embodiments, the engineered (e.g., hypoimmunogenic) epithelial cell can be used to treat a thyroid disease or skin disease.

In some embodiments, the cell is an iPSC-derived hepatocyte that is engineered to contain modifications (e.g., genetic modifications) described herein. In some embodiments, the cell is a primary hepatocyte that is engineered to contain modifications (e.g., genetic modifications) described herein. In some embodiments, the engineered (e.g., hypoimmunogenic) epithelial cell can be used to treat a variety of indications with allogenic cell therapy, including any as described herein, e.g., Section V. In some embodiments, the engineered (e.g., hypoimmunogenic) hepatocyte cell can be used to treat liver disease.

In some embodiments, the cells that are engineered or modified as provided herein are cells from a healthy subject, such as a subject that is not known or suspected of having a particular disease or condition to be treated. For instance, if cells beta islet cells are isolated or obtained from a donor subject, such as for treating diabetes, the donor subject is a healthy subject if the subject is not known or suspected of suffering from diabetes or another disease or condition.

### 1. Primary Cells

In some embodiment the cells that are engineered as provided herein comprise cells derived from primary cells obtained or isolated from one or more individual subjects or donors. In some embodiments, the cells are derived from a pool of isolated primary cells obtained from one or more (e.g., two or more, three or more, four or more, five or more, ten or more, twenty or more, fifty or more, or one hundred or more) different donor subjects. In some embodiments, the primary cells isolated or obtained from the plurality of different donor subjects (e.g., two or more, three or more, four or more, five or more, ten or more, twenty or more, fifty or more, or one hundred or more) are pooled together in a batch and are engineered in accord with the provided methods.

In some embodiments, the primary cells are from a pool of primary cells from one or more donor subjects that are different than the recipient subject (e.g., the patient administered the cells). The primary cells can be obtained from 1, 2, 3, 4, 5, 6, 7, 8, 9, 10, 20, 50, 100 or more donor subjects and pooled together. The primary cells can be obtained from 1 or more, 2 or more, 3 or more, 4 or more, 5 or more, 6 or more, 7 or more, 8 or more, 9 or more, 10, or more 20 or more, 50 or more, or 100 or more donor subjects and pooled together. In some embodiments, the primary cells are harvested from one or a plurality of individuals, and in some instances, the primary cells or the pool of primary T cells are cultured in vitro. In some embodiments, the primary cells or the pool of primary T cells are engineered or modified in accord with the methods provided herein.

In some embodiments, the methods include obtaining or isolating a desired type of primary cell (e.g., T cells, NK cells, NKT cells, endothelial cell, islet cell, beta islet cell, hepatocyte or other primary cells as described herein) from individual donor subjects, pooling the cells to obtain a batch of the primary cell type, and engineering the cells by the methods provided herein. In some embodiments, the methods include obtaining or isolating a desired type of primary cell (e.g., T cells, NK cells, endothelial cell, beta islet cell, hepatocyte or other primary cells as described herein), engineering cells of each of the individual donors by the methods provided herein, and pooling engineered (modified) cells of at least two individual samples to obtain a batch of engineered cells of the primary cell type.

In some embodiments, the primary cells are isolated or obtained from an individual or from a pool of primary cells isolated or obtained from more than one individual donor. The primary cells may be any type of primary cell described herein, including any described in Section II.C.3. In some embodiments, the primary cells are selected from T cells, NK cells, beta islet cells, endothelial cells, epithelial cells such as RPE, thyroid, skin, or hepatocytes. In some embodiments, the primary cells from an individual donor or a pool of individual donors are engineered to contain modifications (e.g., genetic modifications) described herein.

### 2. Generation of Induced Pluripotent Stem Cells

In some embodiments, the cells that are engineered as provided herein are induced pluripotent stem cells or are engineered cells that are derived from or differentiated from induced pluripotent stem cells. The generation of mouse and human pluripotent stem cells (generally referred to as iPSCs; miPSCs for murine cells or hiPSCs for human cells) is generally known in the art. As will be appreciated by those in the art, there are a variety of different methods for the generation of iPCSs. The original induction was done from mouse embryonic or adult fibroblasts using the viral introduction of four transcription factors, Oct3/4, Sox2, c-Myc and Klf4; see Takahashi and Yamanaka Cell 126:663-676 (2006), hereby incorporated by reference in its entirety and specifically for the techniques outlined therein. Since then, a number of methods have been developed; see Seki et al, World J. Stem Cells 7(1); 116-125 (2015) for a review, and Lakshmipathy and Vermuri, editors, Methods in Molecular Biology: Pluripotent Stem Cells, Methods and Protocols, Springer 2013, both of which are hereby expressly incorporated by reference in their entirety, and in particular for the methods for generating hiPSCs (see for example Chapter 3 of the latter reference).

Generally, iPSCs are generated by the transient expression of one or more reprogramming factors” in the host cell, usually introduced using episomal vectors. Under these conditions, small amounts of the cells are induced to become iPSCs (in general, the efficiency of this step is low, as no selection markers are used). Once the cells are “reprogrammed”, and become pluripotent, they lose the episomal vector(s) and produce the factors using the endogeneous genes.

As is also appreciated by those of skill in the art, the number of reprogramming factors that can be used or are used can vary. Commonly, when fewer reprogramming factors are used, the efficiency of the transformation of the cells to a pluripotent state goes down, as well as the “pluripotency”, e.g., fewer reprogramming factors may result in cells that are not fully pluripotent but may only be able to differentiate into fewer cell types.

In some embodiments, a single reprogramming factor, OCT4, is used. In other embodiments, two reprogramming factors, OCT4 and KLF4, are used. In other embodiments, three reprogramming factors, OCT4, KLF4 and SOX2, are used. In other embodiments, four reprogramming factors, OCT4, KLF4. SOX2 and c-Myc, are used. In other embodiments, 5, 6 or 7 reprogramming factors can be used selected from SOKMNLT: SOX2, OCT4 (POU5F1), KLF4, MYC, NANOG, LIN28, and SV40L T antigen. In general, these reprogramming factor genes are provided on episomal vectors such as are known in the art and commercially available.

In some embodiments, the hosts cells used for transfecting the one or more reprogamming factors are non-pluripotent stem cells. In general, as is known in the art, iPSCs are made from non-pluripotent cells such as, but not limited to, blood cells, fibroblasts, etc., by transiently expressing the reprogramming factors as described herein. In some embodiments, the non-pluripotent cells, such as fibroblasts, are obtained or isolated from one or more individual subjects or donors prior to reprogamming the cells. In some embodiments, iPSCs are made from a pool of isolated non-pluripotent stems cells, e.g., fibroblasts, obtained from one or more (e.g., two or more, three or more, four or more, five or more, ten or more, twenty or more, fifty or more, or one hundred or more) different donor subjects. In some embodiments, the non-pluripotent cells, such as fibroblasts, are isolated or obtained from a plurality of different donor subjects (e.g., two or more, three or more, four or more, five or more, ten or more, twenty or more, fifty or more, or one hundred or more), pooled together in a batch, reprogrammed as iPSCs and are engineered in accord with the provided methods.

In some embodiments, the iPSCs are derived from, such as by transiently transfecting one or more reprogramming factors into cells from a pool of non-pluripotent cells (e.g., fibroblasts) from one or more donor subjects that are different than the recipient subject (e.g., the patient administered the cells). The non-pluripotent cells (e.g., fibroblasts) to be induced to iPSCs can be obtained from 1, 2, 3, 4, 5, 6, 7, 8, 9, 10, 20, 50, 100 or more donor subjects and pooled together. The non-pluripotent cells (e.g., fibroblasts) can be obtained from 1 or more, 2 or more, 3 or more, 4 or more, 5 or more, 6 or more, 7 or more, 8 or more, 9 or more, 10, or more 20 or more, 50 or more, or 100 or more donor subjects and pooled together. In some embodiments, the non-pluripotent cells (e.g., fibroblasts) are harvested from one or a plurality of individuals, and in some instances, the non-pluripotent cells (e.g., fibroblasts) or the pool of non-pluripotent cells (e.g., fibroblasts) are cultured in vitro and transfected with one or more reprogramming factors to induce generation of iPSCs. In some embodiments, the non-pluripotent cells (e.g., fibroblasts) or the pool of non-pluripotent cells (e.g., fibroblasts) are engineered or modified in accord with the methods provided herein. In some embodiments, the engineered iPSCs or a pool of engineered iPSCs are then subjected to a differentiation process for differentiation into any cells of an organism and tissue.

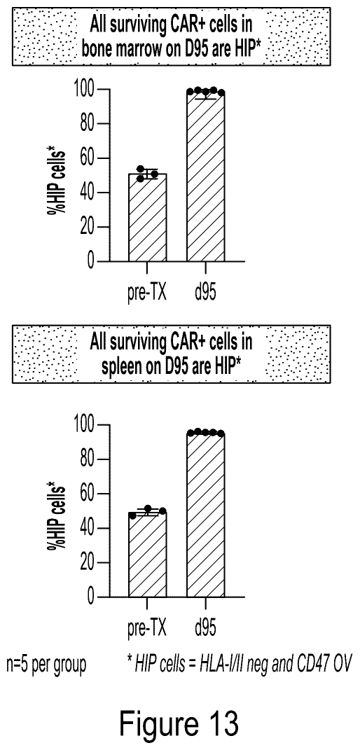

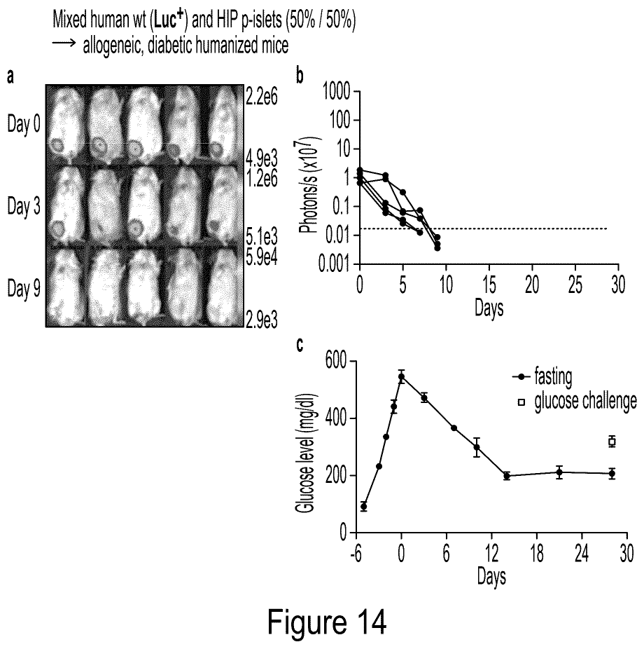

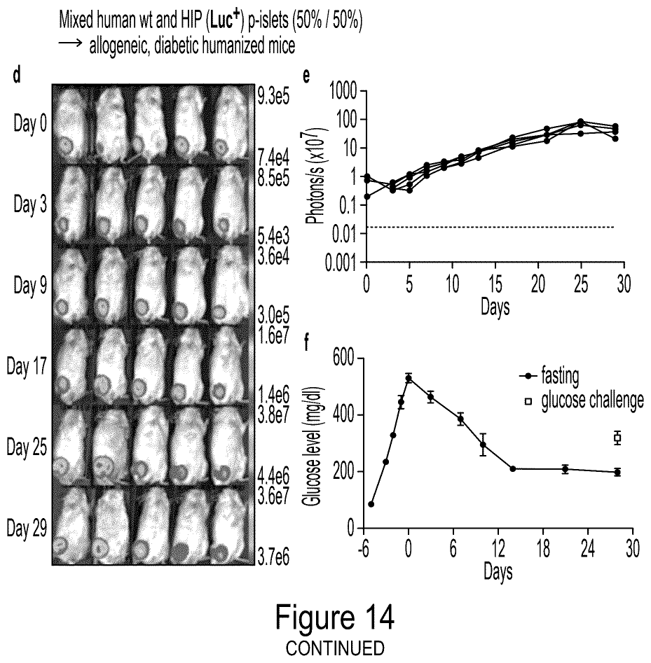

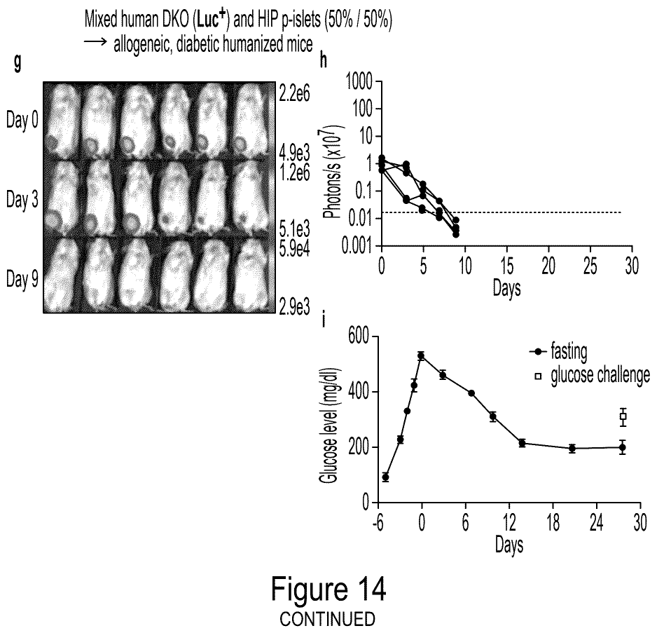

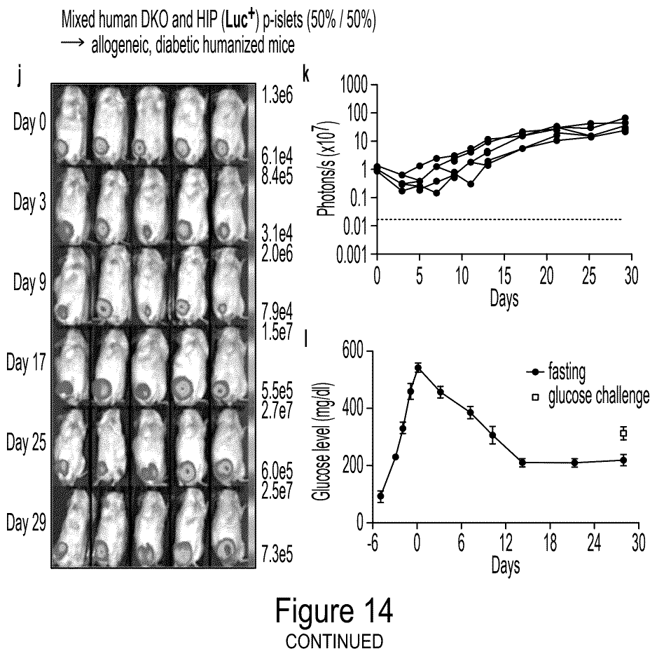

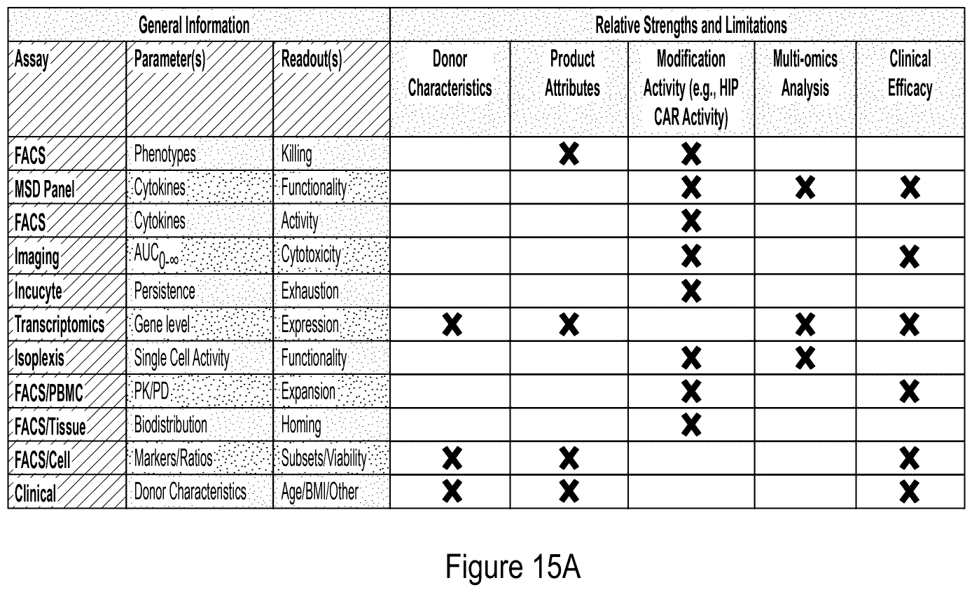

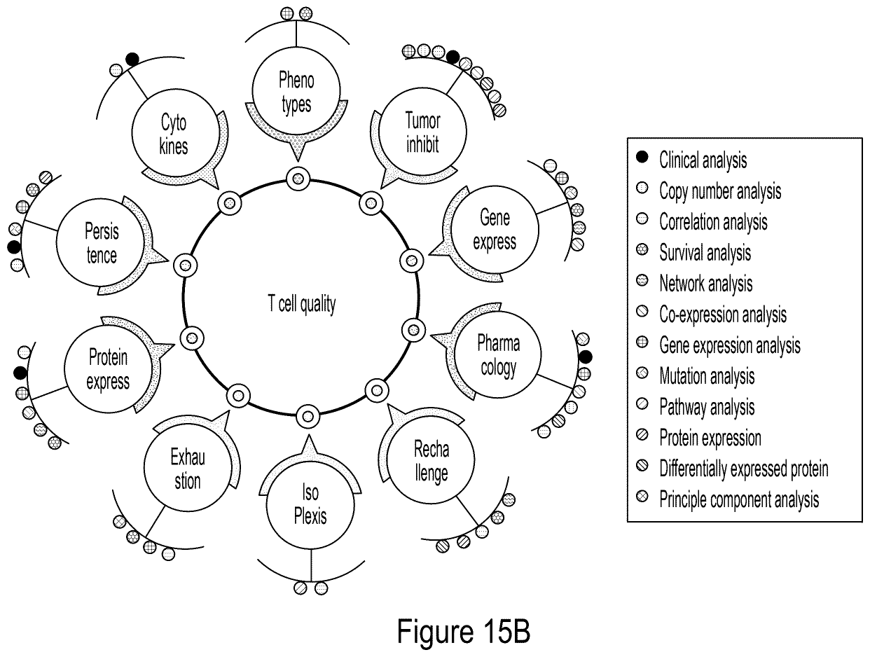

Once the engineered iPSCs cells have been generated, they may be assayed for their hypoimmunogenicity and/or retention of pluripotency as is described in WO2016183041 and WO2018132783. In some embodiments, hypoimmunogenicity is assayed using a number of techniques as exemplified in FIG. 13 and FIG. 15 of WO2018132783. These techniques include transplantation into allogeneic hosts and monitoring for hypoimmunogenic pluripotent cell growth (e.g., teratomas) that escape the host immune system. In some instances, hypoimmunogenic pluripotent cell derivatives are transduced to express luciferase and can then followed using bioluminescence imaging. Similarly, the T cell and/or B cell response of the host animal to such cells are tested to confirm that the cells do not cause an immune reaction in the host animal. T cell responses can be assessed by Elispot, ELISA, FACS, PCR, or mass cytometry (CYTOF). B cell responses or antibody responses are assessed using FACS or Luminex. Additionally or alternatively, the cells may be assayed for their ability to avoid innate immune responses, e.g., NK cell killing, as is generally shown in FIGS. 14 and 15 of WO2018132783.

In some embodiments, the immunogenicity of the cells is evaluated using T cell immunoassays such as T cell proliferation assays, T cell activation assays, and T cell killing assays recognized by those skilled in the art. In some cases, the T cell proliferation assay includes pretreating the cells with interferon-gamma and coculturing the cells with labelled T cells and assaying the presence of the T cell population (or the proliferating T cell population) after a preselected amount of time. In some cases, the T cell activation assay includes coculturing T cells with the cells outlined herein and determining the expression levels of T cell activation markers in the T cells.

In vivo assays can be performed to assess the immunogenicity of the cells outlined herein. In some embodiments, the survival and immunogenicity of engineered or modified iPSCs is determined using an allogeneic humanized immunodeficient mouse model. In some instances, the engineered or modified iPSCs are transplanted into an allogeneic humanized NSG-SGM3 mouse and assayed for cell rejection, cell survival, and teratoma formation. In some instances, grafted engineered iPSCs or differentiated cells thereof display long-term survival in the mouse model.

Additional techniques for determining immunogenicity including hypoimmunogenicity of the cells are described in, for example, Deuse et al., Nature Biotechnology, 2019, 37, 252-258 and Han et al., Proc Natl Acad Sci USA, 2019, 116(21), 10441-10446, the disclosures including the figures, figure legends, and description of methods are incorporated herein by reference in their entirety.

Similarly, the retention of pluripotency is tested in a number of ways. In some embodiments, pluripotency is assayed by the expression of certain pluripotency-specific factors as generally described herein and shown in FIG. 29 of WO2018132783. Additionally or alternatively, the pluripotent cells are differentiated into one or more cell types as an indication of pluripotency.

Once the engineered pluripotent stem cells (engineered iPSCs) have been generated, they can be maintained in an undifferentiated state as is known for maintaining iPSCs. For example, the cells can be cultured on Matrigel using culture media that prevents differentiation and maintains pluripotency. In addition, they can be in culture medium under conditions to maintain pluripotency.

Any of the pluripotent stem cells described herein can be differentiated into any cells of an organism and tissue. In an aspect, provided herein are engineered cells that are differentiated into different cell types from iPSCs for subsequent transplantation into recipient subjects. Differentiation can be assayed as is known in the art, generally by evaluating the presence of cell-specific markers. As will be appreciated by those in the art, the differentiated engineered (e.g., hypoimmunogenic) pluripotent cell derivatives can be transplanted using techniques known in the art that depends on both the cell type and the ultimate use of these cells. Exemplary types of differentiated cells and methods for producing the same are described below. In some embodiments, the iPSCs may be differentiated to any type of cell described herein, including any described in Section II.C.3. In some embodiments, the iPSCs are differentiated into cell types selected from T cells, NK cells, beta islet cells, endothelial cells, epithelial cells such as RPE, thyroid, skin, or hepatocytes. In some embodiments, host cells such as non-pluripotent cells (e.g., fibroblasts) from an individual donor or a pool of individual donors are isolated or obtained, generated into iPSCs in which the iPSCs are then engineered to contain modifications (e.g., genetic modifications) described herein and then differentiated into a desired cell type.

### 3. Cell Type

It will be understood from the disclosure herein that embodiments of the present disclosure may be applied to a number of different cell types. It will be understood that embodiments concerning any cell type described herein may be readily and appropriately combined with embodiments describing safety switches, as well as embodiments describing HIP cells, CAR cells and other modified/gene edited cells as described herein.

Methods for profiling a population of cells for donor capability as described anywhere herein may be performed using primary (e.g., genome-edited) cells. Where the cell therapy product being manufactured is to be comprised of stem cell derived cells, methods for profiling a population of cells for donor capability as described anywhere herein may also be performed using (e.g., genome-edited) stem cells (i.e., pre-differentiation) and/or using stem cell derived cells (i.e., post-differentiation).

### a. Beta-Islet Cells

The pancreas contains clusters of cells, known as islets, that produce hormones. There are several different types of cells in an islet. For example, alpha cells produce glucagon; delta cells produce somatostatin; and beta cells produce insulin. A sample of primary pancreatic islet cells may comprise at least alpha cells: delta cells and beta cells.

Beta-islet cells as described herein to be used in a cell therapy product may be present with alpha cells and delta cells in the cell therapy product. Islet cells to be used in a cell therapy product may be profiled for donor capability at any stage of the manufacturing process of the cell therapy product. In some embodiments, a cell therapy product comprises up to 5, 10, 15, 20, 25, 35, or 40% alpha cells. In some embodiments, a cell therapy product comprises up to 10, 15, 20, 25, 35, 40, 45, 50, 55, 60, 65, 70, 75, or 80% beta cells. In some embodiments, a cell therapy product comprises up to 1, 2, 3, 4, 5, 6, 7, 8, 9, 10, 15, 20, or 25% delta cells.

Beta-islet cells to be used in a cell therapy product may be profiled for donor capability at any stage of the manufacturing process of the cell therapy product.

Beta-islet cells used in a cell therapy product may be primary (e.g., genome-edited) pancreatic islet cells. Methods for profiling a population of cells for donor capability as described any where herein may be performed on primary (e.g., genome-edited) pancreatic islet cells.

As described elsewhere herein, beta-islet cells used in a cell therapy product may be pluripotent stem cell (iPSC)-derived beta-islet cells. Methods for profiling a population of cells for donor capability as described anywhere herein may also be performed on stem cells capable of differentiating to form beta-islet cells. Methods for profiling a population of cells for donor capability as described anywhere herein may also be performed on stem cell derived (e.g., genome-edited) beta-islet cells.

Relevant information concerning beta-islet cells as referred to in the context of the present disclosure is known in the art, including certain information regarding desired features of beta-islet cells when used for cell therapy. It will be understood that embodiments concerning beta-islet cells described herein may be readily and appropriately combined with embodiments describing HIP cells (e.g., exhibiting reduced expression of one or more molecules of the MHC class I and/or MHC class II molecules and increased expression of at least one tolerogenic factor), as well as embodiments describing safety switches, and other modified/gene edited cells as described herein. Beta-islet cells to be used in a cell therapy product may be profiled for donor capability at any stage of the editing process during manufacturing of the cell therapy product.

The beta-islet cells described herein may be used to treat or prevent a disease in a subject.

In some embodiments, the cells that are engineered or modified as provided herein are primary beta islet cells (also referred to as pancreatic islet cells or pancreatic beta cells). In some embodiments, the primary beta islet cells are isolated or obtained from one or more individual donor subjects, such as one or more individual healthy donor (e.g., a subject that is not known or suspected of, e.g., not exhibiting clinical signs of, a disease or infection). As will be appreciated by those in the art, methods of isolating or obtaining beta islet cells from an individual can be achieved using known techniques. Provided herein are engineered primary beta islet cells that contain modifications (e.g., genetic modifications) described herein for subsequent transplantation or engraftment into subjects (e.g., recipients).

In some embodiments, beta islet cells are obtained (e.g., harvested, extracted, removed, or taken) from a subject or an individual. In some embodiments, primary beta islet cells are produced from a pool of beta islet cells such that the beta islet cells are from one or more subjects (e.g., one or more human including one or more healthy humans). In some embodiments, the pool of primary beta islet cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects. In some embodiments, the donor subject is different from the patient (e.g., the recipient that is administered the therapeutic cells). In some embodiments, the pool of beta islet cells does not include cells from the patient. In some embodiments, one or more of the donor subjects from which the pool of beta islets cells is obtained are different from the patient.

In some embodiments, the cells as provided herein are beta islet cells derived from engineered iPSCs that contain modifications (e.g., genetic modifications) described herein and that are differentiated into beta islet cells. As will be appreciated by those in the art, the methods for differentiation depend on the desired cell type using known techniques. In some embodiments, the cells differentiated into various beta islet cells may be used for subsequent transplantation or engraftment into subjects (e.g., recipients). In some embodiments, pancreatic islet cells are derived from the engineered pluripotent cells described herein. Useful methods for differentiating pluripotent stem cells into beta islet cells are described, for example, in U.S. Pat. No. 9,683,215: U.S. Pat. Nos. 9,157,062; 8,927,280; U.S. Patent Pub. No. 2021/0207099; Hogrebe et al., “Targeting the cytoskeleton to direct pancreatic differentiation of human pluripotent stem cells,” Nat. Biotechnol., 2020, 38:460-470; and Hogrebe et al., “Generation of insulin-producing pancreatic beta cells from multiple human stem cell lines,” Nat. Protoc., 2021, the contents of which are herein incorporated by reference in their entirety,

In some embodiments, the engineered pluripotent cells described herein are differentiated into beta-like cells or islet organoids for transplantation to address type I diabetes mellitus (TIDM). Cell systems are a promising way to address T1DM, see, e.g., Ellis et al, Nat Rev Gastroenterol Hepatol. 2017 Oct:14(10):612-628, incorporated herein by reference. Additionally, Pagliuca et al. (Cell, 2014, 159(2):428-39) reports on the successful differentiation of β-cells from hiPSCs, the contents incorporated herein by reference in its entirety and in particular for the methods and reagents outlined there for the large-scale production of functional human β cells from human pluripotent stem cells). Furthermore, Vegas et al. shows the production of human β cells from human pluripotent stem cells followed by encapsulation to avoid immune rejection by the host; Vegas et al., Nat Med, 2016, 22(3):306-11, incorporated herein by reference in its entirety and in particular for the methods and reagents outlined there for the large-scale production of functional human β cells from human pluripotent stem cells.

In some embodiments, the method of producing a population of engineered pancreatic islet cells from a population of engineered pluripotent cells by in vitro differentiation comprises: (a) culturing the population of engineered iPSCs in a first culture medium comprising one or more factors selected from the group consisting insulin-like growth factor, transforming growth factor, FGF, EGF, HGF, SHH, VEGF, transforming growth factor-b superfamily, BMP2, BMP7, a GSK inhibitor, an ALK inhibitor, a BMP type 1 receptor inhibitor, and retinoic acid to produce a population of immature pancreatic islet cells; and (b) culturing the population of immature pancreatic islet cells in a second culture medium that is different than the first culture medium to produce a population of engineered pancreatic islet cells. In some embodiments, the GSK inhibitor is CHIR-99021, a derivative thereof, or a variant thereof. In some instances, the GSK inhibitor is at a concentration ranging from about 2 mM to about 10 mM. In some embodiments, the ALK inhibitor is SB-431542, a derivative thereof, or a variant thereof. In some instances, the ALK inhibitor is at a concentration ranging from about 1 pM to about 10 pM. In some embodiments, the first culture medium and/or second culture medium are absent of animal serum.

Differentiation is assayed as is known in the art, generally by evaluating the presence of β cell associated or specific markers, including but not limited to, insulin. Differentiation can also be measured functionally, such as measuring glucose metabolism, see generally Muraro et al., Cell Syst. 2016 Oct. 26: 3(4); 385-394.e3, hereby incorporated by reference in its entirety, and specifically for the biomarkers outlined there. Once the beta cells are generated, they can be transplanted (either as a cell suspension or within a gel matrix as discussed herein) into the portal vein/liver, the omentum, the gastrointestinal mucosa, the bone marrow, a muscle, or subcutaneous pouches.

Additional descriptions of pancreatic islet cells including for use in the present technology are found in WO2020/018615, the disclosure of which is herein incorporated by reference in its entirety.

In some embodiments, the population of engineered beta islet cells, such as primary beta islet cells isolated from one or more individual donors (e.g., healthy donors) or endothelial cells differentiated from iPSCs derived from one or more individual donors (e.g., healthy donors), are maintained in culture, in some cases expanded, prior to administration. In certain embodiments, the population of engineered beta islet cells are cryopreserved prior to administration.

Exemplary pancreatic islet cell types include, but are not limited to, pancreatic islet progenitor cell, immature pancreatic islet cell, mature pancreatic islet cell, and the like. In some embodiments, pancreatic cells described herein are administered to a subject to treat diabetes.

In some embodiments, the pancreatic islet cells engineered as disclosed herein, such as primary beta islet cells isolated from one or more individual donors (e.g., healthy donors) or beta islet cells differentiated from iPSCs derived from one or more individual donors (e.g., healthy donors), secretes insulin. In some embodiments, a pancreatic islet cell exhibits at least two characteristics of an endogenous pancreatic islet cell, for example, but not limited to, secretion of insulin in response to glucose, and expression of beta cell markers.

Exemplary beta cell markers or beta cell progenitor markers include, but are not limited to, c-peptide, Pdx1, glucose transporter 2 (Glut2), HNF6, VEGF, glucokinase (GCK), prohormone convertase (PC 1/3), Cdcp1, NeuroD, Ngn3, Nkx2.2, Nkx6.1, Nkx6.2, Pax4, Pax6, Ptfla, Isll, Sox9, Soxl7, and FoxA2.

In some embodiments, the pancreatic islet cells, such as primary beta islet cells isolated from one or more individual donors (e.g., healthy donors) or beta islet cells differentiated from iPSCs derived from one or more individual donors (e.g., healthy donors), produce insulin in response to an increase in glucose. In various embodiments, the pancreatic islet cells secrete insulin in response to an increase in glucose. In some embodiments, the cells have a distinct morphology such as a cobblestone cell morphology and/or a diameter of about 17 pm to about 25 pm.

In some embodiments, the present technology is directed to engineered beta islet cells, such as primary beta islet cells isolated from one or more individual donors (e.g., healthy donors) or beta islet cells differentiated from iPSCs derived from one or more individual donors (e.g., healthy donors), that overexpress a tolerogenic factor (e.g., CD47), have reduced expression or lack expression of one or more MHC class I molecules and/or one or more MHC class II molecules (e.g., one or more MHC class I human leukocyte antigens and/or one or more MHC class II human leukocyte antigens). In some embodiments, the beta islet cells further express one or more complement inhibitors. In certain embodiments, the engineered beta islet cells overexpress a tolerogenic factor (e.g., CD47) and harbor a genomic modification in the B2M gene. In some embodiments, the beta islet cells further express one or more complement inhibitors. In some embodiments, the engineered beta islet cells overexpress a tolerogenic factor (e.g CD47) and harbor a genomic modification in the CIITA gene). In some embodiments, beta islet cells overexpress a tolerogenic factor (e.g., CD47) and harbor genomic modifications that disrupt one or more of the B2M and CIITA and genes.

In some embodiments, the provided engineered beta islet cells evade immune recognition. In some embodiments, the engineered beta islet cells described herein, such as primary beta islet cells isolated from one or more individual donors (e.g., healthy donors) or beta islet cells differentiated from iPSCs derived from one or more individual donors (e.g., healthy donors), do not activate an immune response in the patient (e.g., recipient upon administration). Provided are methods of treating a disease by administering a population of engineered beta islet cells described herein to a subject (e.g., recipient) or patient in need thereof.

In some embodiments, the number of cells administered is at a lower dosage than would be required for immunogenic cells (e.g., a population of cells of the same or similar cell type or phenotype but that do not contain the modifications, e.g., genetic modifications, of the engineered cells, e.g., with endogenous levels of and one or more MHC class I molecules and/or one or more MHC class II molecules expression.

### B. Hepatocytes

Hepatocytes to be used in a cell therapy product may be profiled for donor capability at any stage of the manufacturing process of the cell therapy product.

Hepatocytes used in a cell therapy product may be primary hepatocytes. Methods for profiling a population of cells for donor capability as described anywhere herein may be performed using primary (e.g., genome-edited) hepatocytes.

As described elsewhere herein, hepatocytes used in a cell therapy product may be pluripotent stem cell (iPSC)-derived hepatocytes. Methods for profiling a population of cells for donor capability as described anywhere herein may also be performed on stem cells capable of differentiating to form hepatocytes. Methods for profiling a population of cells for donor capability as described anywhere herein may also be performed on stem cell derived (e.g., genome-edited) hepatocytes.

Relevant information concerning hepatocytes as referred to in the context of the present disclosure is known in the art, including certain information regarding desired features of hepatocytes when used for cell therapy and, for example, may be found from WO2022081760, WO2022164807A2, WO2016200340A1, the contents of which are herein incorporated by reference. It will be understood that embodiments concerning hepatocytes described herein may be readily and appropriately combined with embodiments describing HIP cells (e.g., exhibiting reduced expression of one or more molecules of the MHC class I and/or MHC class II molecules and increased expression of at least one tolerogenic factor), as well as embodiments describing safety switches, and other modified/gene edited cells as described herein. Hepatocytes to be used in a cell therapy product may be profiled for donor capability at any stage of the editing process during manufacturing of the cell therapy product.

The hepatocytes described herein may be used to treat or prevent a disease in a subject.

In some embodiments, the cells that are engineered or modified as provided herein are primary hepatocytes. In some embodiments, the primary hepatocytes are isolated or obtained from one or more individual donor subjects, such as one or more individual healthy donor (e.g., a subject that is not known or suspected of, e.g., not exhibiting clinical signs of, a disease or infection). As will be appreciated by those in the art, methods of isolating or obtaining hepatocytes from an individual can be achieved using known techniques. Provided herein are engineered primary hepatocytes that contain modifications (e.g., genetic modifications) described herein for subsequent transplantation or engraftment into subjects (e.g., recipients). In some embodiments, engineered primary hepatocytes can be administered as a cell therapy to address loss of the hepatocyte functioning or cirrhosis of the liver.

In some embodiments, primary hepatocytes are obtained (e.g., harvested, extracted, removed, or taken) from a subject or an individual. In some embodiments, primary hepatocytes are produced from a pool of hepatocytes such that the hepatocytes are from one or more subjects (e.g., one or more human including one or more healthy humans). In some embodiments, the pool of primary hepatocytes is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects. In some embodiments, the donor subject is different from the patient (e.g., the recipient that is administered the therapeutic cells). In some embodiments, the pool of hepatocytes does not include cells from the patient. In some embodiments, one or more of the donor subjects from which the pool of hepatocytes is obtained are different from the patient.

In some embodiments, the cells as provided herein are hepatocytes differentiated from engineered iPSCs that contain modifications (e.g., genetic modifications) described herein and that are differentiated into hepatocyte. As will be appreciated by those in the art, the methods for differentiation depend on the desired cell type using known techniques. In some embodiments, the cells differentiated into a hepatocyte may be used for subsequent transplantation or engraftment into subjects (e.g., recipients). In some embodiments, engineered hepatocytes differentiated from pluripotent stem cells can be administered as a cell therapy to address loss of the hepatocyte functioning or cirrhosis of the liver.

In some embodiments, engineered pluripotent cells containing modifications described herein are differentiated into hepatocytes. There are a number of techniques that can be used to differentiate engineered pluripotent cells into hepatocytes; see for example, Pettinato et al., doi: 10.1038/spre32888, Snykers et al., Methods Mol Biol, 2011 698:305-314, Si-Tayeb et al., Hepatology, 2010, 51:297-305 and Asgari et al, Stem Cell Rev, 2013, 9(4):493-504, all of which are incorporated herein by reference in their entirety and specifically for the methodologies and reagents for differentiation. Differentiation can be assayed as is known in the art, generally by evaluating the presence of hepatocyte associated and/or specific markers, including, but not limited to, albumin, alpha fetoprotein, and fibrinogen. Differentiation can also be measured functionally, such as the metabolization of ammonia, LDL storage and uptake. ICG uptake and release, and glycogen storage.

In some embodiments, the population of engineered hepatocytes, such as primary heptatocytes isolated from one or more individual donors (e.g., healthy donors) or hepatocytes differentiated from iPSCs derived from one or more individual donors (e.g., healthy donors), are maintained in culture, in some cases expanded, prior to administration. In certain embodiments, the population of hepatocytes are cryopreserved prior to administration.

In some embodiments, the present technology is directed to engineered hepatocytes, such as primary hepatocytes isolated from one or more individual donors (e.g., healthy donors) or hepatocytes differentiated from iPSCs derived from one or more individual donors (e.g., healthy donors), that overexpress a tolerogenic factor (e.g., CD47), and have reduced expression or lack expression of one or more MHC class I molecules and/or one or more MHC class II molecules (e.g., one or more MHC class I human leukocyte antigens and/or one or more MHC class II human leukocyte antigens).

In some embodiments, the provided engineered hepatocytes evade immune recognition. In some embodiments, the engineered hepatocytes described herein, such as primary hepatocytes isolated from one or more individual donors (e.g., healthy donors) or hepatocytes differentiated from iPSCs derived from one or more individual donors (e.g., healthy donors), do not activate an immune response in the patient (e.g., recipient upon administration). Provided are methods of treating a disease by administering a population of engineered hepatocytes described herein to a subject (e.g., recipient) or patient in need thereof.

The cells used in embodiments disclosed herein may be hepatocytes, for example a population of hepatocytes. It will be understood that any reference to “a cell” e.g., “a hepatocyte” below also applies to “a population of cells” e.g., “a population of hepatocytes” as described in the present application.

Hepatocytes are the chief functional cells of the liver. Hepatocytes are compacted around a central vein and the portal triad (consisting of the bile ducts, hepatic vein and hepatic artery) are found at the edge of the lobule. While hepatocytes form the majority of the cells in the lobule, several other cell types are essential to form the ductal and vasculature networks, and immune surveillance of the organ. Other major cell types in the liver includes bile duct cells (cholangiocytes), liver sinusoidal endothelial cells, vasculature endothelial cells, immune cells including Pit cells, Kupffer cells and hepatic stellate cells. Within the liver lobules, hepatocytes are distributed to 3 different zones, determined by their proximity to the central vein or the portal triad located at the end of the lobule. Hepatocytes in the different zones are exposed to different niche environments and play specific functional roles in the liver. They can be distinguished by the expression of different markers, glucose and lipid metabolic functions.

Hepatocytes perform an astonishing number of metabolic, endocrine and secretory functions. Roughly 80% of the mass of the liver is contributed by hepatocytes. Hepatocyte function can be assessed by glucose storage, ion uptake, bile salt secretion, amino acid metabolic function, urea synthesis and drug metabolic function test.

The term “hepatocyte” refers to a type of cell that generally makes up 70-80% of the cytoplasmic mass of the liver. Hepatocytes are involved in protein synthesis, protein storage and transformation of carbohydrates, synthesis of cholesterol, bile salts and phospholipids, and detoxification, modification and excretion of exogenous and endogenous substances. The hepatocyte also initiates the formation and secretion of bile. Hepatocytes manufacture serum albumin, fibrinogen and the prothrombin group of clotting factors and are the main site for the synthesis of lipoproteins, ceruloplasmin, transferrin, complement and glycoproteins. In addition, hepatocytes have the ability to metabolize, detoxify, and inactivate exogenous compounds such as drugs and insecticides, and endogenous compounds such as steroids.

The drug metabolizing function of hepatocytes is mediated by cytochrome P450s (CYPs). CYPs constitute the major enzyme family capable of catalyzing the oxidative biotransformation of most drugs. 90% of drugs are metabolized by six major CYPs (CYP3A4/5, CYP2C9, CYP2C19, CYP1A2, CYP2B6 and CYP2D6). CYP function can be enhanced by induction with specific drugs. CYP function and drug induction response is a major critical criterion used to assess the functional maturity of lab-made hepatocytes. While hepatocytes have been derived using various methods from embryonic and fetal tissues, the maturity and functionality of these hepatocytes as determined by the activities of the 6 major CYPs and their response to drug induction showed that they were functionally immature. Therefore, current methods known in the field of deriving hepatocytes do not solve the issue of obtaining large numbers of functionally mature hepatocytes for both industrial and clinical applications.

Cell populations disclosed herein may include hepatocytes and/or hepatocyte progenitors. In some instances, cell populations may be highly enriched for hepatocytes and/or hepatocyte progenitors. By “highly enriched”, it is meant that the cell type(s) of interest will be 70% or more, 75% or more, 80% or more, 85% or more, 90% or more of the cell composition, for example, about 95% or more, or 98% or more of the cell composition. In other words, the population may be a substantially pure composition of the cell type(s) of interest. In some instances, cell populations of interest may include crude preparations. In some instances, cell populations may be prepared from dissociated tissue, filtered or unfiltered. Cell populations containing hepatocytes and/or hepatocyte progenitors may, e.g., depending on the method of isolation and/or preparation, include or exclude various non-hepatocyte cell types including but not limited to e.g., hepatic non-parenchymal cells (NPCs), non-hepatocyte liver associated cells (e.g., stellate cells, Kupffer cells, endothelial cells, biliary cells, etc.), immune cells (e.g., WBCs), RBCs, etc. In some instances, cell populations may be pure or essentially pure preparations of hepatocytes and/or hepatocyte progenitors.

Hepatocytes as disclosed herein may be obtained from a regenerative cell source in the liver.

In vivo, the liver is one of the most regenerative organs of the human body. The human liver can lose up to ⅔ of its mass, maintains its critical functions and recover to the original mass within 8-15 days. Studies have shown during the recovery from a partial liver hepatectomy, hepatocytes from all regions of the liver proliferate, including bile duct cells. Recent lineage tracing experiments have identified proliferative hepatocytes in different regions of the liver, near the central vein or the portal triad during both normal liver homeostasis and liver injuries. Other than hepatocytes, potential proliferative cells have been identified in the bile duct regions that could replenish damage hepatocytes during injury. Therefore, hepatocytes disclosed herein may be obtained through isolation of adult hepatic stem cells from the adult human liver.

The multi-cellular origin of liver stem cells suggests different methods can be used to isolate and derive stem cells from the liver. The ability to successfully isolate and expand liver stem cells and further differentiate them into functional hepatocytes for meaningful repopulation in an injured liver to deliver clinical benefit has become a top priority for liver stem cell biologists. To date, two groups have reported isolating stem cells from adult liver tissue that could be stably expanded in vitro for the long term. However, the hepatocytes derived from these stem cells are functionally immature and are unsuitable for use in clinical and industrial applications.

In some embodiments, the differentiated hepatocyte is differentiated from a liver stem cell isolated from a healthy human or from an iPSC.

Liver stem cells or iPSCs may be obtained from healthy patients.

In some embodiments, hepatocytes or hepatocyte cell populations may be prepared from one or more mammalian livers, such as e.g., human liver. In some instances, a cell population or multiple cell populations, or the engineered cells, including all the engineered cells of a population of multiple cell populations, may all be derived or prepared from a single human liver, such as a single cadaveric donor liver. The cells of a cell population may be all of one species (e.g., human, mouse, rat, pig, NHP, etc.) or may be a mixture of two or more species (i.e., a xenogeneic mixture). Sources of liver will vary and may include but are not limited to e.g., resected liver tissue, cadaveric human liver, chimeric (e.g., humanized) liver, bioreactor liver, and the like. Cell populations may be prepared from liver, including whole livers and liver portions, according to and/or including any convenient method, such as but not limited to e.g., dissociation, perfusion, filtration, sorting, and the like. In some instances, all or essentially all of the hepatocytes or human hepatocytes of a cell population, may be derived from a single donor liver or a portion of a single donor liver. In some instances, all or essentially all of the hepatocytes or human hepatocytes of a cell population, may be derived from a multiple different donor livers or portion of multiple different donor livers. In some instances, multiple cell populations may be derived from a single donor liver, including e.g., where the primary human hepatocytes collected from a single human donor liver are expanded many fold, including 2× or more, 5× or more, 10× or more, 20× or more, 50× or more, 100× or more, etc. to generate a plurality of cell populations, e.g., useful in treating a plurality of subjects.

In some instances, hepatocytes or hepatocyte cell populations may be prepared from cultured hepatocytes and/or cultured hepatocyte progenitors. In some instances, cell populations may be prepared from primary hepatic cell preparations, including e.g., cell populations prepared from human liver that include primary human hepatocytes (PHH). In certain embodiments, the cell population may include hepatocytes isolated using standard techniques for any source. e.g., from human donors. In certain embodiments, the hepatocytes are PHH isolated from screened cadaveric donors, including fresh PHH or cryopreserved PHH. In some instances, PHH of a cell population have undergone no or a minimal number of cell cycles/divisions since isolation from a liver, including but not limited to e.g., 1 or less, 2 or less, 3 or less, 4 or less, 5 or less, 6 or less, 7 or less, 8 or less, 9 or less, 10 cycles/divisions or less.

In some instances, cell populations containing hepatocytes and/or hepatocyte progenitors may be prepared from cells that are not immortalized cell lines or not cells lines that are otherwise essentially perpetually propagated. For example, hepatocytes and/or hepatocyte progenitors of a cell population may be derived from primary liver cells and the progeny of primary liver cells, including e.g., the non-immortalized progeny of primary liver cells.

In some instances, cell populations may include, or may specifically exclude, hepatocyte progenitors. As used herein, the terms “hepatocyte progenitors” and “progenitors of hepatocytes” or the like, generally refer to cells from which hepatocytes are derived and/or cells that are differentiated into hepatocytes. In some instances, hepatocyte progenitors may be committed progenitors, meaning the progenitors will essentially only differentiate into hepatocytes. In some instances, hepatocyte progenitors may have varied potency and may be e.g., pluri-, multi-, or totipotent progenitors. Hepatocyte progenitors may include or be derived from stem cells, induced pluripotent stem cells (iPSCs), embryonic stem (ES) cells, hepatocyte-like cells (HLCs), and the like. In some instances, hepatocyte progenitors may be derived from mature hepatocytes and/or other non-hepatocyte cells, e.g., through dedifferentiation of hepatocytes and/or transdifferentiation of other hepatic or non-hepatic cell types.

Hepatocytes or hepatocyte cell population, or subpopulation, disclosed herein, including expanded cell populations of hepatocytes, may be derived or descended from multiple individual cells, including e.g., multiple individual hepatocytes obtained from a single donor or multiple individual hepatocytes obtained from multiple donors. Where a population of primary cell is derived from a single donor, such multiple individual cells share essentially the same donor genome but are, however, not clonally derived, not monoclonal, and may, in some instances, contain certain differences from one another, including e.g., different genetic variations, different epigenetic variations, different zonation in the donor liver, differences in gene expression, etc. Accordingly, in contrast to clonally-derived cell populations, cell populations expanded from a plurality of individual primary hepatocytes, including primary hepatocytes from a single donor or multiple donors, may be referred to as non-monoclonal or, in some instances, such expanded cells may be referred to as polyclonal or non-clonally expanded. In some instances, genetic modification of the present disclosure may be performed on a population individual primary hepatocytes (or the progeny thereof) to generate a non-monoclonal population of engineered hepatocytes and such cells may be expanded to generate an expanded population of non-monoclonal engineered hepatocytes. In some instances, a population of hepatocytes may be expanded to generate an essentially polyclonal population which is subsequently genetically modified to generate an expanded population of non-monoclonal engineered hepatocytes.

Some desired features of hepatocytes when used for cell therapy are described herein.

In some embodiments, the hepatocytes and/or hepatocyte progenitors, and/or the livers, subjects, and/or cell cultures from which such hepatocytes and/or hepatocyte progenitors are derived, may be healthy hepatocytes and/or hepatocyte progenitors. By “healthy hepatocytes and/or hepatocyte progenitors”, as used herein, is meant that the cells display a normal hepatocyte phenotype and/or genotype essentially free of functional and/or genetic deficiencies or defects in, or that would affect, normal liver and/or hepatocyte associated functions, as described in WO22164807A2 (the contents of which are incorporated herein by reference in their entirety). Hepatocyte-associated functions include those functions primarily or exclusively carried out by hepatocytes in the liver, such as e.g., liver metabolism (e.g., hepatocyte metabolism), ammonia metabolism, amino acid metabolism (inc., bio-synthesis and/or catabolism), detoxification, liver protein (e.g., albumin, fibrinogen, prothrombin, clotting factor (e.g., factor V, VII, IX, X, XI, and XII), protein C, protein S, antithrombin, lipoprotein, ceruloplasmin, transferrin, complement protein) synthesis. Hepatocytes and/or hepatocyte progenitors may be healthy before, during, and/or after genetic modification(s) as described herein. For example, in some instances, a hepatocyte and/or hepatocyte progenitor may be a healthy cell prior to and after genetic modification, e.g., to functionally integrate a heterologous trans gene and/or modify one or more endogenous loci, of the cell. In some instances, hepatocytes and/or hepatocyte progenitors are healthy following correction of a defective disease-associated allele or locus.

Healthy hepatocytes and/or hepatocyte progenitors will generally exclude those cells harboring a genetic aberration associated with a liver- associated monogenic disease, including but not limited to e.g., genetic cholestatic disorders, Wilson's disease, hereditary hemochromatosis, tyrosinemia, al antitrypsin deficiency, urea cycle disorders, Crigler-Najjar syndrome, familial amyloid polyneuropathy, primary hyperoxaluria type 1, atypical haemolytic uremic syndrome-1, and the like. Accordingly, healthy hepatocytes and/or hepatocyte progenitors may contain normal genes/alleles (i.e., non-disease associated genes/alleles, i.e., not contain disease-associated genes/alleles), at loci and/or genes corresponding with liver-associated monogenic diseases, such as but not limited to e.g., ABCB11 (BSEP), AGXT, ARG, ASL, ASS, ATP7B, ATP8B1 (aka FIC1), CFH, CPS, FAH, HAMP, HFE, JAG1, JH, MDR3 (ABCB4), NAGS, OTC, PI, SLC40A1, TFR2, TTR, UGT1 A1, and the like. Further examples and description of genes corresponding with liver- associated monogenic diseases may be found in Fagiuoli et al. J Hepatol (2013) 59(3):595-612; the disclosure of which is incorporated herein by reference in its entirety. Cells harboring one or more genetic aberrations associated with a liver-associated monogenic disease may be referred to herein as “disease”, “diseased”, “disease-associated”, “dysfunctional”, or “defective” cells, or the like.

In some embodiments, the cells used herein are hepatocyte-like cells with enhanced ureagenesis capability. Ureagenesis refers to the process by which a cell converts ammonia to produce urea using the urea cycle. In particular embodiments, the hepatocyte-like cells develop and exhibit enhanced ureagenesis in vitro. In contrast to previous methods that rely on variable in vivo processes to develop hepatocyte-like cells with improved ureagenesis, the cells provided herein exhibit enhanced ureagenesis in vitro as a result of an in vitro differentiation process in the presence of one more ureagenesis enhancers. One of skill in the art can assess enhanced ureagenesis of the subject hepatocyte-like cells prior to transplantation, thereby advantageously reducing potential batch-to-batch variability associated with hepatocyte-like cells using existing methods.

As used herein, the term “enhanced ureagenesis capability” refers an improved ability to convert ammonia to urea by the urea cycle as compared to a reference. Ureagenesis can be measured using any suitable assay known in the art, for example, colorimetric urea assays (e.g., QuantiChrom Assay Kit by BiosAssay Systems). In some embodiments, enhanced ureagenesis capability is compared to a reference cell. In particular embodiments, the reference cell is a mature hepatocyte-like cell differentiated in the absence of one or more of the ureagenesis enhancers provided herein. In some embodiments, enhanced ureagenesis capability of the mature hepatocyte-like cell differentiated in the presence of a particular ureagenesis enhancer is at least 10%, 15%, 20%, 25%, 30%, 35%, 40%, 45%, 50%, 55%, 60%, 65%, 70%, 75%, 85%, 90%, 95% or 99% more ureagenesis as compared to a reference mature hepatocyte-like cell differentiated in the absence of the ureagenesis enhancer. In exemplary embodiments, the subject mature hepatocyte-like cell provided herein exhibits ureagenesis at a level comparable to a wildtype mature hepatocyte. In exemplary embodiments, the subject the mature hepatocyte-like cell provided herein exhibits at least 50%, 55%, 60%, 65%, 70%, 75%, 85%, 90%, 95% or 99% ureagenesis as compared to a reference wild-type mature hepatocyte.

In some embodiments, the hepatocyte-like cell exhibits stimulation of ureagenesis activity when stimulated with ammonia. For example, the hepatocyte-like cell exhibits a ureagenesis rate of at least 3, 4, 5, 6, 8, 10, 12, 15, 20, or 25 nmol/min/10⁶ cells following stimulation with 5 mM or 10 mM ammonia. In some embodiments, the hepatocyte like cell exhibits a ureagenesis rate following stimulation with 5 mM or 10 mM ammonia of between 3 and 50 nmol/min/10⁶ cells, between 3 and 40 nmol/min/10⁶ cells, between 3 and 30 nmol/min/10⁶ cells, between 4 and 50 nmol/min/10⁶ cells, between 4 and 40 nmol/min/10⁵ cells, between 4 and 30 nmol/min/10⁶ cells, between 5 and 50 nmol/min/10⁶ cells, between 5 and 40 nmol/min/10⁶ cells, between 5 and 30 nmol/min/10⁶ cells, between 6 and 50 nmol/min/10⁶ cells, between 6 and 40 nmol/min/10⁶ cells, between 6 and 30 nmol/min/10⁶ cells, between 8 and 50 nmol/min/10⁶ cells, between 8 and 40 nmol/min/10⁶ cells, between 8 and 30 nmol/min/10⁶ cells, between 10 and 50 nmol/min/10⁶ cells, between 10 and 40 nmol/min/10⁶ cells, between 10 and 30 nmol/min/10⁶ cells, between 12 and 50 nmol/min/10⁶ cells, between 12 and 40 nmol/min/10⁶ cells, between 12 and 30 nmol/min/106 cells, between 15 and 50 nmol/min/10⁶ cells, between 15 and 40 nmol/min/10⁶ cells, between 15 and 30 nmol/min/10⁶ cells, between 20 and 50 nmol/min/10⁶ cells, between 20 and 40 nmol/min/10⁶ cells, or between 20 and 30 nmol/min/10⁶ cells.

In some embodiments, the hepatocyte-like cell with enhanced ureagenesis capability exhibits increased expression of one or more urea cycle pathway enzymes. Exemplary urea cycle pathway enzymes include, but are not limited to, carbamoylphosphate synthetase I (CPS1), omithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin. In particular embodiments, the hepatocyte-like cell exhibits expression of one of the following urea cycle enzymes: CPS1, NAGS, ARG1, ASL, ASS1, or OTC. In some embodiments, the hepatocyte-like cell exhibits increased RNA transcript expression of one or more urea cycle pathway enzymes. In exemplary embodiments, the hepatocyte-like cell exhibits increased protein expression of one or more urea cycle pathway enzymes. In some embodiments, the mature hepatocyte-like cell exhibits increased protein expression of one or more urea cycle pathway enzymes at an amount that is at least 10%, 15%, 20%, 25%, 30%, 35%, 40%, 45%, 50%, 55%, 60%, 65%, 70%, 75%, 85%, 90%, 95% or 99% more than a reference mature hepatocyte-like cell differentiated in the absence of a ureagenesis enhancer. In some embodiments, the subject mature hepatocyte-like cell exhibits a similar expression level of one or more urea cycle pathway enzymes as compared to a reference wild-type mature hepatocyte. In exemplary embodiments, the subject mature hepatocyte-like cell exhibits at least 50%, 55%, 60%, 65%, 70%, 75%, 85%, 90%, 95% or 99% the level of expression of one or more urea pathway enzymes as compared to a reference wild-type mature hepatocyte.

As used herein, a “mature hepatocyte-like cell” refers to a cell that exhibits one or more characteristics of a mature hepatocyte including, but not limited to: albumin secretion, α-1 antitrypsin (A1AT) secretion, cytochrome p450 activity, glycogen synthesis capability and/or storage capability, lipid (e.g., low density lipoprotein (LDL)) uptake and/or storage capability, indocyanine green (ICG) uptake and/or clearance capability, and gamma-glutamyl transpeptide activity. In exemplary embodiments, the mature hepatocyte-like cells provided herein exhibit enhanced ureagenesis capability and at least one or more of other characteristics of a mature hepatocyte disclosed herein. In exemplary embodiments, the subject mature hepatocyte-like cell exhibits enhanced ureagenesis capability and 2, 3, 4, 5, 6, 7, 8, 9 or 10 additional characteristics of a mature hepatocyte disclosed herein.

In some embodiments, the mature hepatocyte-like cell exhibits albumin secretion. In some embodiments, the mature hepatocyte-like cell secretes albumin at an amount that is at least 10%, 15%, 20% , 25%, 30%, 35%, 40%, 45%, 50%, 55%, 60%, 65%, 70%, 75%, 85%, 90%, 95% or 99% more than a reference mature hepatocyte-like cell differentiated in the absence of a ureagenesis enhancer. In exemplary embodiments, the mature hepatocyte-like cell exhibits at least 50%, 55%, 60%, 65%, 70%, 75%, 85%, 90%, 95% or 99% the level of albumin secretion of a reference wild-type mature hepatocyte (e.g., a primary human hepatocyte). Albumin secretion can be assessed using any suitable technique include, for example, enzyme-linked immunosorbent (ELISA) assays and immunohistochemistry techniques (see, e.g., Wu et al., Cell Stem Cell 14: 394-403 (2014), which are incorporated herein by reference, particularly in parts pertinent to albumin secretion assessment). In certain embodiments, the mature hepatocyte-like cell exhibits increased expression of the albumin gene (ALB).

In some embodiments, the mature hepatocyte-like cell exhibits α-1 antitrypsin (A1AT) secretion. In some embodiments, the mature hepatocyte-like cell secretes A1AT at an amount that is at least 10%, 15%, 20%, 25%, 30%, 35%, 40%, 45%, 50%, 55%, 60%, 65%, 70%, 75%, 85%, 90%, 95% or 99% more than a reference mature hepatocyte-like cell differentiated in the absence of a ureagenesis enhancer. In exemplary embodiments, the mature hepatocyte-like cell exhibits at least 50%, 55%, 60%, 65%, 70%, 75%, 85%, 90%, 95% or 99% the level of A1AT secretion of a reference wild-type mature hepatocyte (e.g., a primary human hepatocyte). A1AT secretion can be assessed using any suitable technique include, for example, enzyme-linked immunosorbent (ELISA) assays and immunohistochemistry techniques (see, e.g., Wu et al., Cell Stem Cell 14: 394-403 (2014), which is incorporated herein by reference, particularly in parts pertinent to A1AT secretion assessment). In certain embodiments, the mature hepatocyte-like cell exhibits increased expression of the α-1 antitrypsin gene (SERPINA1). In some embodiments, the mature hepatocyte-like cell expresses the SERPINA1 gene at an amount that is at least 10%, 15%, 20%, 25%, 30%, 35%, 40%, 45%, 50%, 55%, 60%, 65%, 70%, 75%, 85%, 90%, 95% or 99% more than a reference mature hepatocyte-like cell differentiated in the absence of a ureagenesis enhancer. In exemplary embodiments, the mature hepatocyte-like cell expresses the SERPINA1 gene at least 50%, 55%, 60%, 65%, 70%, 75%, 85%, 90%, 95% or 99% the level of A1AT secretion of a reference wild-type mature hepatocyte (e.g., a primary human hepatocyte).

In some embodiments, the mature hepatocyte-like cell exhibits coagulation Factor V secretion. In some embodiments, the mature hepatocyte-like cell secretes Factor V at an amount that is at least 10%, 15%, 20%, 25%, 30%, 35%, 40%, 45%, 50%, 55%, 60%, 65%, 70%, 75%, 85%, 90%, 95% or 99% more than a reference mature hepatocyte-like cell differentiated in the absence of a ureagenesis enhancer. In exemplary embodiments, the mature hepatocyte-like cell exhibits at least 50%, 55%, 60%, 65%, 70%, 75%, 85%, 90%, 95% or 99% the level of Factor V secretion of a reference wild-type mature hepatocyte (e.g., a primary human hepatocyte). Factor V secretion can be assessed using any suitable technique include, for example, enzyme-linked immunosorbent (ELISA) assays and immunohistochemistry techniques. In certain embodiments, the mature hepatocyte-like cell exhibits increased expression of the coagulation Factor V gene (F5).

In some embodiments, the mature-hepatocyte-like cell exhibits glycogen synthesis capability and/or storage capability. In some embodiments, the mature hepatocyte-like cell exhibits at least 10%, 15%, 20%, 25%, 30%, 35%, 40%, 45%, 50%, 55%, 60%, 65%, 70%, 75%, 85%, 90%, 95% or 99% more glycogen synthesis capability and/or storage capability than a reference mature hepatocyte-like cell differentiated in the absence of a ureagenesis enhancer. In exemplary embodiments, the mature hepatocyte-like cell exhibits at least 50%, 55%, 60%, 65%, 70%, 75%, 85%, 90%, 95% or 99% the level glycogen synthesis capability and/or storage capability of a reference wild-type mature hepatocyte (e.g., a primary human hepatocyte). Glycogen synthesis capability and/or storage capability can be assessed, for example, using immunohistochemistry techniques (see, e.g., Du et al., Cell Stem Cell 14: 394-403 (2014)), which is incorporated herein by reference, particularly in parts pertinent to glycogen synthesis capability and/or storage capability assessment.

In some embodiments, the mature-hepatocyte-like cell exhibits lipid (e.g., VLDL, LDL, and HDL) uptake and/or storage capability. In particular embodiments, the mature-hepatocyte-like cell exhibits low-density lipoprotein (LDL) uptake and/or storage capability. In some embodiments, the mature hepatocyte-like cell exhibits at least 10%, 15%, 20%, 25%, 30%, 35%, 40%, 45%, 50%, 55%, 60%, 65%, 70%, 75%, 85%, 90%, 95% or 99% more lipid uptake and/or storage than a reference mature hepatocyte-like cell differentiated in the absence of a ureagenesis enhancer. In exemplary embodiments, the mature hepatocyte-like cell exhibits at least 50%, 55%, 60%, 65%, 70%, 75%, 85%, 90%, 95% or 99% the level of lipid uptake and/or storage of a reference wild-type mature hepatocyte (e.g., a primary human hepatocyte).

In some embodiments, the mature-hepatocyte-like cell exhibits ICG uptake and/or clearance capability. In some embodiments, the mature hepatocyte-like cell at least 10%, 15%, 20%, 25%, 30%, 35%, 40%, 45%, 50%, 55%, 60%, 65%, 70%, 75%, 85%, 90%, 95% or 99% greater ICG uptake and/or clearance than a reference mature hepatocyte-like cell differentiated in the absence of a ureagenesis enhancer. In exemplary embodiments, the mature hepatocyte-like cell exhibits at least 50%, 55%, 60%, 65%, 70%, 75%, 85%, 90%, 95% or 99% the level of lipid uptake and/or storage of a reference wild-type mature hepatocyte (e.g., a primary human hepatocyte). Lipid uptake/storage capability and ICG uptake and/or clearance capability can be assessed using immunohistochemistry techniques as described, for example in Huang et al., Cell Stem Cell 14: 370-384 (2014); and Wang et al., Cell Stem Cell 19: 449-461 (2016), which are incorporated herein by reference, particularly in parts pertinent to ICG activity assessment.

In some embodiments, the mature hepatocyte-like cell exhibits cytochrome p450 activity. Such cells may exhibit activity of one or more cytochrome p450 family members, including CYP1A1, CYPIA2, CYP2C9, CYP2C19, CYP2B6, CYP2D6, CYP3A4, and/or CYP3A7. In certain embodiments, the mature hepatocyte-like cell exhibits gene or protein expression of one or more cytochrome p450 family members. In some embodiments, the mature hepatocyte-like cell exhibits at least 50%, 55%, 60%, 65%, 70%, 75%, 85%, 90%, 95% or 99% the level of cytochrome p450 activity of a reference wild-type mature hepatocyte (e.g., a primary human hepatocyte). Cytochrome p450 activity can be measured using nucleic acid quantitative analysis techniques (e.g., qPCR), luminescent assays (see, e.g., Kim et al., Biomol Ther: 23(5); 486-492 (2015); and P450-Glo assay kit, Promega), or liquid chromatography/mass spectrometry techniques (see, e.g., Du et al., Cell Stem Cell 14: 394-403 (2014), and Lahoz et al., Methods in Molecular Biology 806:97-97 (2012)), all of which are incorporated herein by reference, particularly in parts pertinent to cytochrome p450 activity assessment.

In some embodiments, the mature hepatocyte-like cell exhibits asialoglycoprotein receptor expression (e.g., ASGR1 and/or ASGR2 expression. In exemplary embodiments, the mature hepatocyte-like cell exhibits at least 50%, 55%, 60%, 65%, 70%, 75%, 85%, 90%, 95% or 99% the level of asialoglycoprotein receptor expression of a reference wild-type mature hepatocyte (e.g., a primary human hepatocyte).

In some embodiments, the mature hepatocyte-like cell exhibits alpha-fetoprotein (AFP) expression. In exemplary embodiments, the mature hepatocyte-like cell exhibits at least 50%, 55%, 60%, 65%, 70%, 75%, 85%, 90%, 95% or 99% the level of alpha-fetoprotein expression of a reference wild-type mature hepatocyte (e.g., a primary human hepatocyte).

In some embodiments, the mature hepatocyte-like cell exhibits gamma-glutamyl transpeptidase activity. In exemplary embodiments, the mature hepatocyte-like cell exhibits at least 50%, 55%,60%,65%, 70%, 75%,85%,90%,95% or 99% the level of gamma-glutamyl transpeptidase activity of a reference wild-type mature hepatocyte (e.g., a primary human hepatocyte). Gamma-glutamyl transpeptidase activity can be assessed, for example, using immunohistochemistry techniques (see, e.g., Woo et al., Gastroenterology 42:602-611 (2012)), which is incorporated herein by reference, particularly in parts pertinent to gamma-glutamyl transpeptidase activity assessment.

In some embodiments, the mature hepatocyte-like cell exhibits SOX9 expression. In exemplary embodiments, the mature hepatocyte-like cell exhibits at least 50%, 55%, 60%, 65%, 70%, 75%, 85%, 90%, 95%, or 99% the level of SOX9 expression of a reference wild-type mature hepatocyte (e.g., a primary human hepatocyte). In some embodiments, the mature hepatocyte-like cell exhibits increased SOX9 expression compared to a reference wild-type mature hepatocyte.

In some embodiments, the mature hepatocyte-like cell exhibits keratin, type I cytoskeletal 18 (KRT18) expression. In some embodiments, the mature hepatocyte-like cell exhibits at least 50%, 55%, 60%,65%, 70%, 75%, 85%,90%, 95%, or 99% the level of KRT18 expression of a reference wild-type mature hepatocyte (e.g., a primary human hepatocyte). In some embodiments, the mature hepatocyte-like cell exhibits increased KRT18 expression compared to a reference wild-type mature hepatocyte.

In some embodiments, the mature hepatocyte-like cell exhibits HNF4A (e.g., HNF4a and HNF4α) expression. In some embodiments, the mature hepatocyte-like cell exhibits at least 50%, 55%, 60%, 65%, 70%, 75%, 85%, 90%, 95%, or 99% the level of HNF4A expression of a reference wild-type mature hepatocyte (e.g., a primary human hepatocyte). In some embodiments, the mature hepatocyte-like cell exhibits increased HNF4A expression compared to a reference wild-type mature hepatocyte.

In some embodiments, the mature hepatocyte-like cell exhibits G6PC expression. In some embodiments, the mature hepatocyte-like cell exhibits at least 50%, 55%, 60%, 65%, 70%, 75%, 85%, 90%, 95% or 99% the level of G6PC expression of a reference wild-type mature hepatocyte (e.g., a primary human hepatocyte). In some embodiments, the mature hepatocyte-like cell exhibits increased G6PC expression compared to a reference wild-type mature hepatocyte.

In some embodiments, the differentiated hepatocytes described herein are characterized by expression of hepatocyte markers HNF4a, ALB, CYP2C9, and can also express the terminal differentiation marker PEPCK1. The tight junction marker ZO1 may be expressed between differentiated cells. In some embodiments, the differentiated hepatocytes may also express functional CYP enzymes, including CYP3A4, CYP2C9, CYP2B6, CYP1 A2, CYP1A1, CYP2D6, CYP3A7, and CYP2E1. The differentiated hepatocytes may exhibit other features of mature hepatocytes, including functional glucose metabolism (determined by PAS staining for glycogen synthesis and storage), functional lipid metabolism (determined by low-density lipoprotein (LDL) uptake assay), indocyanine green (ICG) uptake, and albumin secretion. ICG uptake is a medical diagnostic test used for diagnosis of hepatic functions, and is used demonstrates that hepatocytes have functional transporter functions to transport ions in and out of the hepatocytes.

The hepatocytes disclosed herein may exhibit metabolic functions of the major CYPs, CYP1A2, CYP3A4, CYP2B6 and CYP2C9, which accounts for majority of compound metabolism activities of the liver. More importantly, the hepatocytes may be responsive to known CYP inducing drugs such as Rifampicin.

In some embodiments, the differentiated hepatocyte comprises at least one of the following characteristics: (i) expresses the markers HNF4a and ALB;
(ii) expresses at least 1, 2, 3, 4, 5, 6, 7 or 8 of the CYP enzymes selected from CYP3A4, CYP2C9, CYP2B6, CYP1A2, CYP1A1, CYP2D6, CYP3A7, and CYP2E1;
(iii) have at least 1, 2 or 3 of the following functions: a) functional glucose metabolism; b) functional lipid metabolism; c) functional albumin secretion;
(vi) indocyanine green (ICG) uptake; and
(vii) forms tight junctions when cultured with other differentiated hepatocytes under conditions sufficient to form an epithelium.

The differentiated hepatocyte can also express at least one of the terminal differentiation markers PEPCK1 or TAT. The differentiated hepatocyte has inducible CYP function for at least 2, 3, or 4 of the CYPs selected from CYP3A4, CYP2C9, CYP2B6, CYP1A2, CYP1A1, CYP2D6. CYP3A7, and CYP2E1. The differentiated hepatocyte can also store glycogen and uptake low density lipoprotein. Properties of such hepatocytes are described in WO2016200340A1, the contents of which are incorporated herein by reference in their entirety.

Isolation and Long Term Culture of Human Liver Stem Cells may be performed according to the following method, as described in WO2016200340A1, the contents of which are incorporated herein by reference in their entirety.

As described in the ‘Materials & Methods’ in WO2016200340A1:

Collagenase digestion Solution: DEME/F12 (Gibco) with 10 mM Hepes, 5% FBS (Clonetech), 2 mg/ml collagenase (Sigma, C-5138). Warm collagenase solution to 37 degree C., filter sterilize after the collagenase has gone into solution through a 0.2 micron filter. Make this media fresh (collagenase will inactivate at high temperatures and over long periods of time).

Washing solution: DEME/F12 medium with 10 mM HEPES (Gibco), 100U/ml Pen/Strep (Gibco), 100 μg/ml gentamicin (Gibco), filter sterilize solution through a 0.2 micron filter.

Coating medium: Growth factor reduced matrigel (Corning) 10%, diluted advanced F12/DMEM reduced serum medium(Gibco).

Liver stem cell culture medium: Advanced F12/DMEM reduced serum medium (1:1)(Gibco. 12643), 10 mM HEPES (Gibco), 100 U/ml Pen/Strep (Gibco), 2 mM L-Glutamine (Gibco), 1% N2 (Gibco), 2% B27 (Gibco), 50 ng/ml EGF (Millipore), 250 ng/ml R-Spondin1(R&D), 2 μM SB431542 (Tocris)), 1 μM Jagged-1(188-204) (Anaspec), 2 μM T3 (3,3′,5-Triiodo-L-Thyronine)(Sigma), 0.1 μM Cholera endotoxin (Sigma), 10 mM Nicotinamide (Sigma), 1.25 μM N-Acetyl-Cysteine (NAC)(Sigma).

Preparation of feeder cell as described in WO2016200340A1: 1. The plates may be coated with 10% matrigel. Matrigel may be diluted with advanced F12/DMEM basal medium. After coating the plates, the plates may be store in 37° C., incubator for 30 min. 2. Vials containing 7×106 cells/vial may be thawed into 3 six well plates with 2 mL medium each well. The cells may be thawed quickly in the waterbath, and swabbed with 70% alcohol before bringing them into a laminar-flow hood. The cells may be added into a 50 mL tube and warm medium was added drop by drop to dilute the cells. 3. The plates may be shaken in the incubator (left and right ×2 & back and front ×2) to evenly distribute the cells. 4. The feeder could be used on the second day.

Liver stem cells may be isolated from fresh human liver tissue. The use of human liver samples for hepatocyte preparation for scientific purposes were approved by Institutional Review Boards (IRBs) and Human Research Ethics committee. Normal liver samples may be obtained from donor livers for transplantation. Cirrhosis liver samples may be obtained from recipient liver for transplantation. The tissue may be cut and minced into small pieces and digested in collagenase solution. The digested cells may be suspended in liver stem cell culture medium. The cells may be pooled and plated on tissue culture plates with feeder cells and allowed to attach. After 7-10 days, cells may be harvested and resuspended as single cells and plated onto the feeders. Hepatic stem cell colonies may start to appear 3 days later. The cells may be subcultured every 7 to 10 days for long term culture. Culture medium may be changed every 2 days.

Single cell derived cell lines may be generated by picking up single colonies on feeder for continual culture. Early passaged liver stem cell may be digested in single cells and retrieved by flow cytometry. The sorted single cell may be cultured in 96 well plate and further split into 48, 24 and 6 wells plates for expansion.

In certain embodiments, the digested early passage cells may be filtered through a 40 μπ strainer and plated at a low density on the culture dish. After 10 to 14 days, single cells should grow to form colonies without merging with other colonies. The single cell derived colonies may be picked for continuing culture on 24 well plates. 1%, 10%, 20%, 30%, 40% and 50% of the cells repopulated in the next passaging.

Single cell derived hepatic stem cell line may be passaged continuously every 7 to 14 days, early passage and late passage may be checked for karyotyping. Cells at both passages may be seen to have normal karyotype. This shows that the stem cell genomes were stable during long term culture.

According to the results described in WO2016200340A1: The hepatic stem cells may express adult stem cell marker SOX9 and hepatocyte markers HNF4a and HNF3P. Bile duct marker KRT19 may be lowly expressed and bile duct marker KRT7 may be undetectable in the cells (protein level). The hepatic stem cell may also expressed other liver stem cell marker EPCAM, CD24, PROM1, FOXA3 and FOXQ 1. During the maintenance of stem cells, colonies of differentiated cells may be observed (−1-2% of total number of colonies). The differentiated liver cells may show strong levels of ECAD, KRT19, KRT7 expression and decreased express SOX9, consistent with the differentiated cells being ductal in nature. Morphologically, undifferentiated cells may be small, round in shape and clustered tightly together. The hepatic stem cells may not be polarized. Sporadic differentiated cells may be larger and flatter in shape. Almost all the cells in culture may be proliferating as all the cells may express KI67 in the cell nucleus. Morphologically, all of them may be small round shape. The undifferentiated cells expressed low levels of KRT19, but may not express detectable levels of KRT7.

Differentiation of liver stem cells to hepatocytes may be performed by the following method, as described in WO2016200340A1, the content of which are incorporated herein by reference in their entirety:

As described in WO2016200340A1, liver Stem cells may be able to differentiate into both hepatocyte and bile duct cell at near 100% efficiency. Hepatocyte differentiations may be conducted in both 2D, 3D and air liquid interface (ALI) format. Bile duct differentiation may be in 3D format.

Hepatocyte differentiation medium may consist of Clonetics™ HCM™ Hepatocyte Culture Medium (Lonza) supplemented with 0.5 μM A83-01 (Tocris), 30 μMdexamethasone(Dex) (Sigma), 20 ng/ml oncostatin M(OSM)(Prospecbio), 0.1 μM γ-secretase inhibitor XXI, also called compound E (Santa cruz), 25 ng/ml Bmp7 and 25 ng/ml Fgf19.

In some embodiments, Clonetics™ HCM™ Hepatocyte Culture Medium (Lonza) could be replaced with advanced DMEM/F12 (1:1) medium with B27, N2 or DMEM/F12 (1:1) medium with 10% FBS.

In some embodiments, γ-secretase inhibitor XXI could be replaced with the Notch pathway inhibitor DAPT, DBZ.

In some embodiments, BMP7, FGF19, and oncostatin M (OSM) were optional. Either one of them could be removed, and the cells will still able to differentiate into hepatocyte with lower efficiency.

### 2D Hepatocyte Differentiation

Hepatic stem cells may be cultured on feeder for 7 to 10 days. After stem cells reach 50% to 70% confluence, cell culture medium may be added with BMP420, 50, 100 ng/ml and Fgf2 10, 20, 50, 100 ng/ml for 3-5 days and changed to hepatocytes differentiation medium to initiate hepatocyte differentiation. The medium may be changed every 2-3 days for 14 days. Prolonged culture in hepatocyte differentiation medium could increase the hepatocyte maturation. The differentiated cells could be continued cultured for more than 30 days in Clonetics™ HMM™ Hepatocyte Maintenance Medium (HMM).

In some embodiments, liver stem cells may be seeded on feeders at high density 1×10 5 cells/cm 2 in stem cell culture medium. When the liver stem cells reach confluency, the culture media may be replaced with the differentiation media. Mature hepatocyte will form after 14 to 21 days.

### 3D Hepatocyte Differentiation

Liver stem cells may be cultured on feeders for 6 to 10 days. The cells subsequently may be retrieved and seeded as single cells in ultra-low attachment cell culture dish at a density of 1×105 cells/cm in human liver stem cell culture medium.

In certain embodiments, cells may be seeded on low attachment 96 well plate at 2-5×104/well. [0240] On the second day, the stem cells may be cultured in liver stem cell medium with BMP4 20, 50, 100ng/m and Fgf2 10,20, 50, 100 ng/ml for another 3-5 days to form sphere structures. Hepatocyte differentiation medium may be used to culture the sphere structures in low attachment plate to initiate hepatocyte differentiation. Medium may be changed every 2-3 days. The mature hepatocytes may be derived in 8th to 14th days of differentiation. Prolonged culture in hepatocyte differentiation medium may increased the 3D hepatocyte maturation. The 3D hepatocytes may be cultured in differentiation medium as long as 30 days with medium changing every 2-3 days. The 3D hepatocyte could be maintained in Clonetics™ HMM™ Hepatocyte Maintenance Medium (HMM) for 2-3 months. Hepatocytes air-liquid interface (ALI) differentiation

Liver stem cells may be plated on 3T3J2 feeders in transwells (Corning, 0.4 pm pore size, polyester membrane) at a density of 3×10 4 cell/cm 2. The cells in transwells may be cultured in liver stem cell medium for 7 to 10 days. The medium inside the inserts may be removed and the medium outside the insert may be changed to hepatocyte differentiation. The cells on air- liquid interface may be cultured for another 14 days with medium changes every 2-3 days. In day 10 to 14, the mature hepatocyte may be derived. Prolonged culture in hepatocyte differentiation medium could increase the hepatocyte maturation. The differentiated cells could be continued cultured for more than 30 days.

All described hepatocyte differentiation methods should produce functional human hepatocytes without fetal and immature hepatocyte markers expression such as AFP (except for hepatocytes derived from LSCs from hepatocarcinoma patients, which do express AFP).

As described in WO2016200340A1, differentiated hepatocytes should have Cytochrome p450 drug metabolism function, glycogen storage and LDL uptake function. The efficiency of 2D and 3D differentiation may be around 80% to 90%. The highest differentiation efficiency of 3D could reach 100% in certain individual 3D hepatocyte spheres. The 3D differentiated hepatocytes may express the hepatocyte terminal differentiation markers PEPCK and TAT. As described in WO2016200340A1, six major CYPs p450 family members may have increased RNA expression.

CYP1A2, 2C9, 2B6, and 3A4/5 function may be determined by metabolic assays. The hepatocytes may also show drug induced response for CYP3A4 and CYP2C9 activity.

Although Hepatic stem cell differentiated hepatocyte as described in WO2016200340A1 displayed many features of primary adult hepatocyte, they may be different in several aspects. Liver stem cell differentiated hepatocyte (dHep) may be smaller than adult primary hepatocyte (aHep) in cell size, aHep size may be 2-10 times larger than dHep. Majority of the dHep may be diploid with single nucleus, but a significant numbers of aHep may be polyploid, and can also be binucleated, aHep CYPs function could only be maintained in vitro for 24-48 hours in cell culture, but dHep CYPs function could be maintained for more than 30 days.

As described in WO2016200340A1, differentiation of liver stem cells to cholangiocytes may be performed by the following method:

Biliary duct differentiation medium may consist of advanced F12/DMEM reduced serum medium (1:1)(Gibco. 12643), 10 mM HEPES(Gibco), 100U/ml Pen/Strep(Gibco), 2 mM L-Glutamine(Gibco), 1% N2(Gibco), 2% B27(Gibco), 10 ng/ml EGF(Millipore), 100 ng/ml Fgf10(R&D).

Tgfp inhibitor and Notch inhibitor may not present in the medium. The bile duct differentiation medium may be Tgfp3 inhibitor and Notch inhibitor free.

In certain embodiments, Tgfp3 and Notch ligand jagged- 1 were added in the differentiation medium to help bile duct forming. Either of them was optional.

Growth factor reduced matrigel may be thawed one day before at 4° C. 50 ul of matrigel was put in one well of the 8 chamber slide. The chamber slides with matrigel were incubated at 37° C. Half an hour later, as described in WO2016200340A1, the matrigle may solidify and form dome shape and jelly like structure on each well. Liver stem cells may be digested by 0.05% trypsin for 30 to 60 seconds. The liver stem cells may be seeded in the growth factor reduced matrigel suspension at a density of 3-5×104

After 3 to 5 days, when the sphere structures may form, the liver stem cell medium may be changed to biliary duct differentiation medium. The differentiation medium may be changed every 2 days. Cholangiocytes may form 14 days later.

As described in WO2016200340A1, the biliary duct differentiation method may gave rise to biliary duct-like 3D structure. This structure mimics a closed duct in which the cells are arranged in a sphere or tubular structure with an enclosed lumen or space surrounded by the cholangiocytes which have tight junctions between them. In some of the biliary duct-like structures mucus can be observed to be secreted into the lumen. All the cells stained positive for biliary markers KRT19 and KRT7 suggesting close to 100% differentiation efficiency. The cells may be fully polarized with microvilli on the luminal apical part of the structures. Nucleus in the cells may be located near the basal membrane. Tight junction marker ZO-1 may be expressed between the cells. The biliary duct organoids may display organization that mimicked in vivo biliary duct structure.

The process of bile duct 3D differentiation can comprise of the following steps: placing extracellular matrix in a suitable container (such as 50-60 μl of matrigel on one well of 8 well chamber slides): solidifying the extracellular matrix (such as matrigel) to form a dome shape jelly-like structure in the suitable container (such as cell culture chamber); placing the liver stem cells (optionally with suitable digestion enzymes such as trypsin) on the extracellular matrix (such as seeding the liver stem cells on top of the matrigel): incubating the extracellular matrix and the liver stem cells and allowing the aggregated cells to form a sphere structure on top of the extracellular matrix (such as matrigel). Without wishing to be bound by theory, it is believed that the extracellular matrix (such as matrigel) may support cells aggregation and form 3D structure, which helps to initiate bile duct differentiation. Furthermore, it is believed that TGF-beta and other cytokine in the extracellular matrix (such as matrigel) may facilitate the sphere structures to further differentiate into bile duct-like structure. Extracellular matrix (such as matrigel) may support the sphere structure formation and assist in the polarization of the sphere structure to further differentiate into bile duct-like structure. Although Hepatic stem cell differentiated cholangiocytes may display many features of primary adult cholangiocytes, they may be different in several aspects. The LSC-derived cholangiocytes may lack cuboidal cholangiocytes which have proliferative potential. No proliferation of LSC cholangiocytes may be observed. While ECAD expression varies in the adult primary cholangiocyte, all the cholangiocyte in the culture may express ECAD.

A liver, such as a human liver, or portion thereof may be obtained, and optionally surgically processed (e.g., to isolate one or more portions or lobe(s) of the liver), for example as described in WO22164807A2 the contents of which are incorporated herein by reference in their entirety. The liver, or portion thereof, is then decellularized by any convenient and appropriate means, including e.g., mechanical cell damage, freeze/thawing, cannulation and retrograde profusion of one or more decellularization reagents (e.g., one or more protease (e.g. trypsin), one or more nuclease (e.g., DNase), one or more surfactants (e.g., sodium dodecyl sulfate, Triton X-100, or the like), one or more hypotonic reagents, one or more hypertonic reagents, combinations thereof, or the like. The decellularized liver, or a portion thereof, may be stored and/or presoaked in a hepatocyte-compatible media. Cell suspension containing ex vivo manipulated hepatocyte-generating cells as described herein may then be applied to the decellularized liver, or portion thereof, by any convenient mechanism, such as e.g., injection, perfusion, topical application (e.g., drop-by-drop), or combination thereof. In some instances, the ex vivo manipulated hepatocyte-generating cells may be present in the cell suspension, for seeding into a prepared scaffold, at any convenient and appropriate concentration, including e.g., a concentration of 1×105 or less to 1×107 or more cells per 50 pL, including but not limited to e.g., 1-2 ×106 cells per 50 pL. Seeded decellularized liver, portions thereof, and/or other acellularized scaffolds may be maintained under suitable conditions for engraftment/attachment and/or expansion of the introduced cells, where such conditions may include suitable humidity, temperature, gas exchange, nutrients, etc. In some instances, a seeded liver, portion thereof, and/or other acellularized scaffold may be maintained in a suitable culture medium a humid environment at or about 37° C., with 5% CO2. Following attachment and/or expansion of seeded and/or generated hepatocytes to or within the decellularized liver, portion thereof, or other acellularized scaffold, the material may be employed for various uses, including e.g., transplantation into a subject in need thereof, such as a human subject with decreased liver function and/or a liver disease. Methods and reagents relating to decellularization of liver, including human livers, and the production of hepatocyte-receptive acellular scaffolds are described in e.g., Mazza et al. Sci Rep 5, 13079 (2015); Mango et al. Adv. Funct. Mater. 2000097 (2020); Shimoda et al. Sci Rep 9, 1543 (2019); Croce et al. Biomolecules. 2019, 9(12):813: as well as U.S. Pat. No. 10,688,221, the disclosures of which are incorporated herein by reference in their entirety.

Collected cell populations produced by the methods as described herein and therapeutic or pharmaceutical compositions thereof may be present in any suitable container (e.g., a culture vessel, tube, flask, vial, cryovial, cryo-bag, etc.) and may be employed (e.g., administered to a subject) using any suitable delivery method and/or device. Such populations of hepatocytes and pharmaceutical compositions may be prepared and/or used fresh or may be cryopreserved. In some instances, populations of hepatocytes and pharmaceutical compositions thereof may be prepared in a “ready-to-use” format, including e.g., where the cells are present in a suitable diluent and/or at a desired delivery concentration (e.g., in unit dosage form) or a concentration that can be readily diluted to a desired delivery concentration (e.g., with a suitable diluent or media). Populations of hepatocytes and pharmaceutical compositions thereof may be prepared in a delivery device or a device compatible with a desired delivery mechanism or the desired route of delivery, such as but not limited to e.g., a syringe, an infusion bag, or the like.

In some instances, the present disclosure includes a plurality of cell therapy doses, e.g., each contained in suitable container, including e.g., where the genetically modified hepatocytes of the plurality of doses are all derived, including expanded, from a hepatocyte population, e.g., a master cell bank, created from a single human donor liver. In some instances, the present disclosure includes a plurality of cell therapy doses, e.g., each contained in suitable container, including e.g., where the genetically modified hepatocytes of the plurality of doses are all derived, including expanded, from a single hepatocyte population. e.g., a master cell bank, created from a plurality (e.g., 2, 2 or more, 3 or less, 3, 3 or more, 4 or less, 4, 4 or more, 5 or less, 5, 5 or more, 6 or less, 6, 6 or more, 7 or less, 7, 7 or more, 8 or less, 8. 8 or more, 9 or less, 9, 9 or more, 10 or less, 10 or more, etc.) human donor livers.

Any of the hepatocyte-like cells produced by the methods described herein can be used treat a subject. In one aspect, provided herein are pharmaceutical compositions that include the subject mature hepatocyte-like cells with enhanced in vitro ureagenesis capability. In some embodiments, the ureagenesis capability of the mature hepatocyte-like cells is assessed prior to use in a pharmaceutical composition. Ureagenesis can be measured using any suitable assay known in the art, for example, colorimetric urea assays (e.g., QuantiCrom Assay Kit by BiosAssay Systems). In exemplary embodiments, the pharmaceutical composition includes mature hepatocyte-like cell that exhibits at least 50%, 55%, 60%, 65%, 70%, 75%, 85%, 90%, 95% or 99% ureagenesis as compared to a reference wild-type mature hepatocyte.

### Cell Differentiation

The subject mature hepatocyte-like cells described herein may be produced by in vitro differentiating a source cell in the presence of a ureagenesis enhancer at a discrete time point during the differentiation period.

In exemplary embodiments, the source cell is differentiated into a hepatocyte-like progenitor cell intermediate and the hepatocyte-like progenitor cell is differentiated into a mature-like hepatocyte in a culture medium that includes one or more ureagenesis enhancers. As used herein, a “hepatocyte-like progenitor cell” refers to a cell capable of differentiating into a mature hepatocyte-like cell and that has one or more characteristics of a hepatocyte progenitor cell. Hepatocyte progenitor cell characteristics include, but are not limited to: EpCAM expression, cytokeratin-19 expression, asialoglycoprotein receptor 1 (ASGPR1) expression, and increased gene expression of AFP, ALB, HNF4A, HNF6, SERPINA1, ALB and/or CYP3A7. In particular embodiments, the increased gene expression of AFP, ALB, HNF4A, HNF6, SERPINA1, ALB and/or CYP3A7 is compared to a reference source cell (e.g., a stem cell, a fibroblast or any source cell described herein). In certain embodiments, the increase in gene expression is at least a 10-fold, 100-fold, 1×10³-fold, 1×10⁴-fold, 1×10⁵-fold, or 1×10⁶-fold increase in gene expression as compared to a reference source cell. In exemplary embodiments, the hepatocyte-like progenitor cell exhibits 1) expression of EpCAM and/or cytokeratin-19; and 2) increased gene expression of AFP, ALB, HNF4A, HNF6, SERPINA1, ALB and/or CYP3A7. In some embodiments, the hepatocyte-like progenitor cell exhibits 1) expression of EpCam and cytokeratin-19; and 2) increased expression of ALB and CYP3A7. Hepatocyte-like progenitor cells also include hepatoblasts-like cells, which are bipotential progenitor cells capable of differentiating into hepatocytes or cholangiocytes. In some embodiments, the one or more ureagenesis enhancers is contacted with the mature hepatocyte-like cell.

In some embodiments, one or more ureagenesis enhancers are contacted with the differentiating cell beginning at a specific day of differentiation. In some embodiments, one or more ureagenesis enhancers are contacted with the differentiating cell beginning at day 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 of differentiation. In some embodiments, one or more ureagenesis enhancers are contacted with the differentiating cell 1, 2, 3, 4, 5, 6, 7, 8, 9, 10, 11, 12, 13, 14, 15, 16, 17, 18, 19, or 20 days prior to differentiation into the mature hepatocyte-like cell. In some embodiments, one or more ureagenesis enhancers are contacted with a mature hepatocyte-like cell that exhibits one or more characteristics of a mature hepatocyte as disclosed herein. In some embodiments, the ureagenesis enhancer is contacted with the differentiating cell (e.g., the hepatocyte-like progenitor cell) for at least 2, 3, 4, 5, 6, 7, 8, 9, 10, 11, 12, 24 or 36 hours. In some embodiments, the ureagenesis enhancer is contact with the differentiating cell for at least 2, 3, 4, 5, 6, 7, 8, 9, 10, 11, 12. 13, or 14 days. In several embodiments, the ureagenesis enhancer is contact with the differentiating cell for at least 1, 2, 3, 4, 5, 6, 7, 8, 9, or 10 weeks.

### Source Cells

Any suitable source cell can be used for producing the subject hepatocyte-like cells including, but not limited to, stem cells (e.g., induced pluripotent stem cells and embryonic stem cells) fibroblasts, gastric epithelial cells, hepatocytes, and ductal cells.

In some embodiments, the source cell is a stem cell. In some embodiments, the source cell is a human stem cell. In some embodiments, the stem cell is a pluripotent stem cell. “Pluripotent stem cells” as used herein have the potential to differentiate into any of the three germ layers: endoderm (e.g., the stomach lining, gastrointestinal tract, lungs, etc.), mesoderm (e.g., muscle, bone, blood, urogenital tissue, etc.) or ectoderm (e.g., epidermal tissues and nervous system tissues). The term “pluripotent stem cells,” as used herein, also encompasses “induced pluripotent stem cells”, or “iPSCs”, a type of pluripotent stem cell derived from a non-pluripotent cell. Examples of parent cells include somatic cells that have been reprogrammed to induce a pluripotent, undifferentiated phenotype by various means. Such “iPS” or “iPSC” cells can be created by inducing the expression of certain regulatory genes or by the exogenous application of certain proteins. Methods for the induction of iPS cells are known in the art. See, e.g., Zhou et al, Stem Cells 27 (11):2667-74 (2009); Huangfu et al., Nature Biotechnol. 26 (7):795 (2008); Woltjen et al, Nature 458 (7239):766-770 (2009); and Zhou et al. Cell Stem Cell 8:381-384 (2009). In certain embodiments, the stem cell is an embryonic stem cell. Methods of hepatocyte differentiation using stem cells are known in the art. See, e.g., Blackford et al., Stem Cells Transl Med. 8(2):124-137 (2019); Hannan et al., Nature Prot. 8(2):430-437 (2013); Liu et al., Sci Transl Med. 3(82):82ra39 (2011); Woo et al., Gastroenterology 42:602-611 (2012); and Carpentier et al., J Clin Invest. 124(11); 4953-4964 (2014), which are incorporated herein by reference, particularly in parts pertinent to hepatocyte-like cell differentiation methods.

In some embodiments, wherein the source cell is an induced pluripotent stem cell, the induced pluripotent stem cell is first differentiated to a definitive endoderm (DE) that expresses one or more of the following genes: CXCR4, SOX17. CER, and/or FOXA2. The DE is then subsequently differentiated into a hepatocyte-like progenitor cell that expresses alpha-1-fetoprotein (AFP). Subsequently, the hepatocyte-like progenitor cell is differentiated into the mature hepatocyte-like cell. See, e.g., Liu et al., Sci Transl Med. 3(82):82ra39 (2011), which is incorporated herein by reference, particularly in parts pertinent to hepatocyte-like cell differentiation. In some embodiments, the hepatocyte-like progenitor cell is differentiated in the presence of one or more of the ureagenesis enhancers described herein to produce the mature hepatocyte-like cell with enhanced ureagenesis capability. In some embodiments, the hepatocyte-like progenitor cell is differentiated to the mature hepatocyte-like cell in a culture medium that includes one or more ureagenesis enhancers and oncostatin-M (OSM). In some embodiments, the culture medium further includes hepatocyte growth factor (HGF). In some embodiments, the mature hepatocyte-like cell is subjected to the one or more ureagenesis enhancers.

In some embodiments, wherein the source cell is a human embryonic stem cell or a human induced pluripotent stem cell, the source cell is first differentiated to a definitive endoderm that expresses SOX17 and/or FOXA2. The definitive endoderm is differentiated into a hepatoblast-like cell that expresses AFP and/or HNF4A. The hepatoblast-like cell is then differentiated into a mature hepatocyte-like cell that express one or a combination of the following genes: AFP, AAT, ALB, and HNF3B. See, e.g., Carpentier et al., J Clin Invest. 124(11); 4953-4964 (2014), which is incorporated herein by reference, particularly in parts pertinent to hepatocyte-like cell differentiation. In some embodiments, the hepatoblast-like cell is differentiated in the presence of one or more of the ureagenesis enhancers described herein to produce the mature hepatocyte-like cell with enhanced ureagenesis capability. In some embodiments, the hepatoblast-like cell is differentiated in a culture medium that includes one or more ureagenesis enhancers, DMSO and HGF. In some embodiments, the mature hepatocyte-like cell is subjected to the one or more ureagenesis enhancers.

In some embodiments, wherein the source cell is a human pluripotent stem cell, the source cell is first differentiated to a definitive endoderm that expresses SOX17 and/or CXCR4. The definite endoderm is differentiated into a hepatic endoderm that expresses EpCAM, cytokeratin 19 and one or a combination of the following genes: AFP. SERPINA2, and HNF4A. The hepatic endoderm is then differentiated into a mature hepatocyte-like cell that express one or a combination of the following genes: AFP, ASGR2, SERPINA2, CYP3A7, and ALB. See, e.g., Blackford et al., Stem Cells Transl Med. 8(2):124-137 (2019), which is incorporated herein by reference, particularly in parts pertinent to hepatocyte-like cell differentiation. In some embodiments, the hepatic endoderm is differentiated in the presence of one or more of the ureagenesis enhancers described herein to produce the mature hepatocyte-like cell with enhanced ureagenesis capability. In some embodiments, the hepatic endoderm is differentiated in a cell culture medium that includes one or more ureagenesis enhancers and oncostatin-M (OSM). In some embodiments, the culture medium further includes hepatocyte growth factor (HGF). In some embodiments, the mature hepatocyte-like cell is subjected to the one or more ureagenesis enhancers.

In some embodiments, the mature hepatocyte-like cell is transdifferentiated from a source cell. Transdifferentiation refers to the direct conversion of a differentiated cell type into another without an intermediary pluripotent stage. Exemplary cells that are capable of transdifferentiation into mature hepatocyte-like cells include, but are not limited to, fibroblasts, gastric epithelial cells, hepatocytes, and ductal cells.

In some embodiments, the mature hepatocyte-like cell is transdifferentiated from a fibroblast. See. e.g., Zhu et al., Nature 508(7494):93-7(2014); Du et al., Cell Stem Cell 14(3):394-403 (2014); and Huang et al., Cell Stem Cell 4(3):370-84, which are incorporated herein by reference, particularly in parts pertinent to methods of transdifferentiation. In such methods, one or more genes useful for fibroblast to hepatocyte reprogramming are transferred to the fibroblast by retroviral transduction delivery techniques and the transduced fibroblast is cultured in medium containing factors favoring the formation of a hepatocyte-like progenitor cell that subsequently differentiates into a mature-hepatocyte like cell. Genes that are useful for fibroblast to hepatocyte reprogramming include, but are not limited to, OCT4, SOX2, KLF4, HNF6, HNF1α, FOXA3, HNF1β and/or HNF4α. In some embodiments, the hepatocyte-like progenitor cell is differentiated in the presence of one or more of the ureagenesis enhancers described herein to produce the mature hepatocyte-like cell with enhanced ureagenesis capability.

In some embodiments, the mature hepatocyte-like cell is transdifferentiated from a mature hepatocyte. In some embodiments, the mature hepatocyte is converted into expandable hepatocyte-like progenitor cells that subsequently differentiate into mature-hepatocyte like cells. In some embodiments, conversion of mature hepatocyte to hepatocyte-like progenitor cell is carried out in a culture medium that includes one or a combination of the following: a Wnt signaling agonist (e.g., CHIR99021), a TGFβ signaling inhibitor (e.g., A82-01 and A83-01) and a ROCK kinase inhibitor (e.g., Y27632, A82-01). See, e.g., Kim et al., J. Hepatol. 70(1):97-107 (2019); and Fu et al., Cell Res. 29(1):8-22 (2019), which are incorporated herein by reference, particularly in parts pertinent to methods of transdifferentiation. In some embodiments, the hepatocyte-like progenitor cell is differentiated in the presence of one or more of the ureagenesis enhancers described herein to produce the mature hepatocyte-like cell with enhanced ureagenesis capability.

In some embodiments, the subject mature hepatocyte-like cell is transdifferentiated from a gastric epithelial cell (e.g., Wang et al., Cell Stem Cell 19(4):449-61 (2016)) or a ductal cell (see, e.g., Huch et al., Cell 160(1-2):299-312 (2015), which are incorporated herein by reference, particularly in parts pertinent to methods of transdifferentiation). In some embodiments, the source cell is transdifferentiated in the presence of one or more of the ureagenesis enhancers described herein to produce the mature hepatocyte-like cell with enhanced ureagenesis capability.

### Ureagenesis Enhancers

The subject mature hepatocyte-like cells provided herein are produced by in vitro differentiating a source cell under one or more conditions that promote enhanced in vitro ureagenesis capability. Small molecule enhancers

In some embodiments, differentiation occurs in at least one culture medium that includes one or more small molecule ureagenesis enhancers. In exemplary embodiments, such a differentiation step occurs after the source cell has differentiated into an intermediate hepatocyte-like progenitor cell. In certain embodiments, the small molecule enhancer is added to a mature hepatocyte-like cell that exhibits one or more characteristics of a mature hepatocyte as disclosed herein. In some embodiments, differentiation in the presence of the one or more small molecule ureagenesis enhancers occurs 1, 2, 3, 4, 5, 6, 7, 8, 9, 10, 11, 12, 13, 14, 15, 16, 17, 18, 19, or 20 days prior to formation of the mature hepatocyte-like cell. In some embodiments, the small molecule ureagenesis enhancer is contact with the differentiating cell for at least 2, 3, 4, 5, 6, 7, 8, 9, 10, 11, 12, 13, or 14 days. In some embodiments, the small molecule ureagenesis enhancer is contact with the differentiating cell for 2, 3, 4, 5, 6, 7, 8, 9, 10, 11, 12, 13, or 14 days. In some embodiments, the small molecule ureagenesis enhancer is contact with the differentiating cell for at least 1, 2, 3, 4, 5, 6, 7, 8, 9, or 10 weeks.

In some embodiments, the small molecule enhancers disclosed herein are included in the culture medium at a concentration of at least 1, 5, 10, 15, 20, 25, 30, 35, 40, 45, 50, 55, 60, 65, 70, 75, 80, 85, 90, 100, 250, 500, or 750 μM. In some embodiments, the small molecule enhancers disclosed herein are included in the culture medium at a concentration of between 1 and 800 μM, between 1 and 750 μM, between 1 and 700 μM, between 1 and 650 μM, between 1 and 600 μM, between 1 and 550 μM, between 1 and 500 μM, between 1 and 500 μM, between 1 and 450 μM, between 1 and 400 μM, between 1 and 350 μM, between 1 and 300 μM, between 1 and 250 μM, between 1 and 200 μM, between 1 and 150 μM, between 1 and 100 μM, between 1 and 50 μM, between 50 and 800 μM, between 50 and 750 μM, between 50 and 700 μM, between 50 and 650 μM, between 50 and 600 μM, between 50 and 550 μM, between 50 and 500 μM, between 50 and 500 μM, between 50 and 450 μM, between 50 and 400 μM, between 50 and 350 μM, between 50 and 300 μM, between 50 and 250 μM, between 50 and 200 μM, between 50 and 150 μM, between 50 and 100 μM, between 100 and 800 μM, between 100 and 750 μM, between 100 and 700 μM, between 100 and 650 μM, between 100 and 600 μM, between 100 and 550 μM, between 100 and 500 μM, between 100 and 500 μM, between 100 and 450 μM, between 100 and 400 μM, between 100 and 350 μM, between 100 and 300 μM, between 100 and 250 μM, between 100 and 200 μM, between 100 and 150 μM, between 200 and 800 μM, between 200 and 750 μM, between 200 and 700 μM, between 200 and 650 μM, between 200 and 600 μM, between 200 and 550 μM, between 200 and 500 μM, between 200 and 500 μM, between 200 and 450 μM, between 200 and 400 μM, between 200 and 350 μM, between 200 and 300 μM, between 200 and 250 μM, between 300 and 800 μM, between 300 and 750 μM, between 300 and 700 μM, between 300 and 650 μM, between 300 and 600 μM, between 300 and 550 μM, between 300 and 500 μM, between 300 and 500 μM, between 300 and 450 μM, between 300 and 400 μM, between 300 and 350 μM, between 400 and 800 μM, between 400 and 750 μM, between 400 and 700 μM, between 400 and 650 μM, between 400 and 600 μM, between 400 and 550 μM, between 400 and 500 μM, between 400 and 500 μM, between 400 and 450 μM, between 500 and 800 μM, between 500 and 750 μM, between 500 and 700 μM, between 500 and 650 μM, between 500 and 600 μM, between 500 and 550 μM, between 600 and 800 μM, between 600 and 750 μM, between 600 and 700 μM, between 600 and 650 μM, between 700 and 800 μM, between 700 and 750 μM, or between 750 and 800 μM.

In some embodiments, the small molecule enhancers disclosed herein are included in the culture medium at a concentration of at least 1, 5, 10, 15, 20, 25, 30, 35, 40, 45, 50, 55, 60, 65, 70, 75, 80, 85, 90, 100, 250, 500, or 750 mM. In some embodiments, the small molecule enhancers disclosed herein are included in the culture medium at a concentration of between 1 and 800 mM, between 1 and 750 mM, between 1 and 700 mM, between 1 and 650 mM, between 1 and 600 mM, between 1 and 550 mM, between 1 and 500 mM, between 1 and 500 mM, between 1 and 450 mM, between 1 and 400 mM, between 1 and 350 mM, between 1 and 300 mM, between 1 and 250 mM, between 1 and 200 mM, between 1 and 150 mM, between 1 and 100 mM, between 1 and 50 mM, between 50 and 800 mM, between 50 and 750 mM, between 50 and 700 mM, between 50 and 650 mM, between 50 and 600 mM, between 50 and 550 mM, between 50 and 500 mM, between 50 and 500 mM, between 50 and 450 mM, between 50 and 400 mM, between 50 and 350 mM, between 50 and 300 mM, between 50 and 250 mM, between 50 and 200 mM, between 50 and 150 mM, between 50 and 100 mM, between 100 and 800 mM, between 100 and 750 mM, between 100 and 700 mM, between 100 and 650 mM, between 100 and 600 mM, between 100 and 550 mM, between 100 and 500 mM, between 100 and 500 mM, between 100 and 450 mM, between 100 and 400 mM, between 100 and 350 mM, between 100 and 300 mM, between 100 and 250 mM, between 100 and 200 mM, between 100 and 150 mM, between 200 and 800 mM, between 200 and 750 mM, between 200 and 700 mM, between 200 and 650 mM, between 200 and 600 mM, between 200 and 550 mM, between 200 and 500 mM, between 200 and 500 mM, between 200 and 450 mM, between 200 and 400 mM, between 200 and 350 mM, between 200 and 300 mM, between 200 and 250 mM, between 300 and 800 mM, between 300 and 750 mM, between 300 and 700 mM, between 300 and 650 mM, between 300 and 600 mM, between 300 and 550 mM, between 300 and 500 mM, between 300 and 500 mM, between 300 and 450 mM, between 300 and 400 mM, between 300 and 350 mM, between 400 and 800 mM, between 400 and 750 mM, between 400 and 700 mM, between 400 and 650 mM, between 400 and 600 mM, between 400 and 550 mM, between 400 and 500 mM, between 400 and 500 mM, between 400 and 450 mM, between 500 and 800 mM, between 500 and 750 mM, between 500 and 700 mM, between 500 and 650 mM, between 500 and 600 mM, between 500 and 550 mM, between 600 and 800 mM, between 600 and 750 mM, between 600 and 700 mM, between 600 and 650 mM, between 700 and 800 mM, between 700 and 750 mM, or between 750 and 800 mM.

In some embodiments, the small molecule enhancer increases expression of one or more urea cycle pathway enzymes. Exemplary urea cycle pathway enzymes include, but are not limited to, carbamoylphosphate synthetase I (CPS1), ornithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin. In some embodiments, the small molecule enhancer increases expression of one of the following urea cycle enzymes: CPS1, NAGS, ARG1 ASL, ASS1, or OTC. In some embodiments, the small molecule enhancer increases RNA transcription expression of one or more urea cycle pathway enzymes. In some embodiments, the hepatocyte-like cell exhibits increased protein expression of one or more urea cycle pathway enzymes. In some embodiments, the small molecule enhancer increases protein expression of one or more urea cycle pathway enzymes at an amount that is at least 10%, 15%, 20%, 25%, 30%, 35%, 40%, 45%, 50%, 55%, 60%, 65%, 70%, 75%, 85%, 90%, 95% or 99% more than a reference mature hepatocyte-like cell differentiated in the absence of the enhancer.

In some embodiments, the small molecule enhancer is an agent that increases intracellular cyclic AMP. Without being bound by any particular theory of operation and as disclosed in the examples provided herein, agents that increase intracellular cyclic AMP increase transcription and translation of enzymes of the urea cycle, thereby increasing the in vitro ureagenesis capacity of the mature hepatocyte-like cell. Agents that increase intracellular cyclic AMP include, but are not limited to, forskolin, glucagon, glucagon-like peptide-1 (GLP-1), glucose-dependent insulinotropic peptide (GIP), phosphodiesterase inhibitors, and analogs of any of the foregoing. Exemplary phosphodiesterase inhibitors include IBMX (3-isobutyl-1-methylxanthine), theophylline, V11294A, rolipram, milrinone, CDP-840, papaverine, sildenafil, tadalafil, roflumilast, amrinone, cilostazol, and dipyridamole.

In exemplary embodiments, the enhancer is forskolin. In some embodiments, the differentiating hepatocyte-like progenitor cell is differentiated in a cell culture medium that includes forskolin. In some embodiments, a hepatocyte-like cell that exhibits one or more characteristics of a mature hepatocyte as described herein is differentiated in a cell culture medium that includes forskolin. In some embodiments, forskolin is included in the culture medium at a concentration of at least 1, 5, 10, 15, 20, 25, 30, 35, 40, 45, 50, 75, or 100 JIM. In some embodiments, forskolin is included in the culture medium at a concentration of at least 1, 5, 10, 15, 20, 25, 30, 35, 40, 45, 50, 75, or 100 mM. In some embodiments, forskolin is included in the culture medium at a concentration of between 0.5 and 800 μM, between 0.5 and 750 μM, between 0.5 and 700 μM, between 0.5 and 650 μM, between 0.5 and 600 μM, between 0.5 and 550 μM, between 0.5 and 500 μM, between 0.5 and 500 μM, between 0.5 and 450 μM, between 0.5 and 400 μM, between 0.5 and 350 μM, between 0.5 and 300 μM, between 0.5 and 250 μM, between 0.5 and 200 μM, between 0.5 and 150 μM, between 0.5 and 100 μM, between 0.5 and 95 μM, between 0.5 and 90 μM, between 0.5 and 85 μM, between 0.5 and 80 μM, between 0.5 and 75 μM, between 0.5 and 70 μM, between 0.5 and 65 μM, between 0.5 and 60 μM, between 0.5 and 55 μM, between 0.5 and 50 μM, between 0.5 and 45 μM, between 0.5 and 40 μM, between 0.5 and 35 μM, between 0.5 and 30 μM, between 0.5 and 25 μM, between 0.5 and 20 μM, between 0.5 and 15 μM, between 0.5 and 10 μM, between 0.5 and 9 μM, between 0.5 and 8 μM, between 0.5 and 7 μM, between 0.5 and 6 μM, between 0.5 and 5 μM, between 0.5 and 2 μM, between 0.5 and 1 μM, between 1 and 30 μM, between 5 and 30 μM, between 10 and 30 μM, between 1 and 20 μM, between 5 and 20 μM, or between 10 and 20 μM. In some embodiments, forskolin is included in the culture medium at a concentration of 5-20 μM. In some embodiments, the cell (e.g., a differentiating hepatocyte-like progenitor cell) is subjected to forskolin for at least 1, 2, 3, 4, 5, 6, 7, 8, 9, 10, 11, 12, 13, 14, 15, 16, 17, 18, 19, 20, 21, 22, 23, or 24 hours. In some embodiments, the differentiating cell is subjected to forskolin for at least 8 hours. In some embodiments, the cell culture medium further includes Oncostatin-M (OSM) and/or hepatocyte growth factor (HGF).

In some embodiments, the small molecule enhancer is vitamin K1. In some embodiments, the vitamin K is vitamin K1. In some embodiments, differentiation occurs in a culture medium that is substantially free of vitamin K2 and/or vitamin K3. Without being bound by any particular theory of operation and as disclosed in the examples provided herein, vitamin K can increase in vitro ureagenesis in mature hepatocyte-like cells. In some embodiments, a differentiating hepatocyte-like progenitor cell is differentiated in a cell culture medium that includes vitamin K1. In some embodiments, a hepatocyte-like cell that exhibits one or more characteristics of a mature hepatocyte as described herein is differentiated in a cell culture medium that includes vitamin K1. In some embodiments, vitamin K1 is included in the culture medium at a concentration of at least 0.05, 0.1, 0.2, 0.3, 0.4, 0.5, 0.75, 1, 2, 3, 4, 5, 10, 15, 20, 25, 30, 35, 40, 45, 50, 55, 60, 65, 70, 75, 80, 85, 90, 100, 250, 500, or 750 μM. In some embodiments, vitamin K is included in the culture medium at a concentration of between 0.05 and 800 μM, between 0.05 and 750 μM, between 0.05 and 700 μM, between 0.05 and 650 μM, between 0.05 and 600 μM, between 0.05 and 550 μM, between 0.05 and 500 μM, between 0.05 and 500 μM, between 0.05 and 450 μM, between 0.05 and 400 μM, between 0.05 and 350 μM, between 0.05 and 300 μM, between 0.05 and 250 μM, between 0.05 and 200 μM, between 0.05 and 150 μM, between 0.05 and 100 μM, between 0.05 and 95 μM, between 0.05 and 90 μM, between 0.05 and 85 μM, between 0.05 and 80 μM, between 0.05 and 75 μM, between 0.05 and 70 μM, between 0.05 and 65 μM, between 0.05 and 60 μM, between 0.05 and 55 μM, between 0.05 and 50 μM, between 0.05 and 45 μM, between 0.05 and 40 μM, between 0.05 and 35 μM, between 0.05 and 30 μM, between 0.05 and 25 μM, between 0.05 and 20 μM, between 0.05 and 15 μM, between 0.05 and 10 μM, between 0.05 and 9 μM, between 0.05 and 8 μM, between 0.05 and 7 μM, between 0.05 and 6 μM, between 0.05 and 5 μM, between 0.05 and 2 μM, between 0.05 and 1 μM, between 0.05 and 0.5 μM, between 0.05 and 0.1 μM, between 0.1 and 5 μM, between 0.5 and 5 μM, or between 1 and 5 μM. In some embodiments, the cell (e.g., a differentiating hepatocyte-like progenitor cell) is subjected to vitamin K1 for at least 1, 2, 3, 4, 5, 6, 7, 8, 9, 10, 11, 12, 13, 14, 15, 16, 17, 18, 19, 20, 21, 22, 23, or 24 hours. In some embodiments, the differentiating cell is subjected to vitamin for at least 8 hours. In some embodiments, arginine is added to the culture medium together with the vitamin K1. In some embodiments, arginine is added at a concentration of 750 μM- 10 mM. In some embodiments, arginine is added to the culture medium at a similar concentration to vitamin K1. In some embodiments, the cell culture medium further includes Oncostatin-M (OSM) and/or hepatocyte growth factor (HGF).

In some embodiments, the small molecule enhancer is vitamin K2. In exemplary embodiments, the vitamin K is vitamin K2. In some embodiments, differentiation occurs in a culture medium that is substantially free of vitamin K1 and/or vitamin K3.

In some embodiments, a differentiating hepatocyte-like progenitor cell is differentiated in a cell culture medium that includes vitamin K2. In embodiments, a hepatocyte-like cell that exhibits one or more characteristics of a mature hepatocyte as described herein is differentiated in a cell culture medium that includes vitamin K2. In some embodiments, vitamin K2 is included in the culture medium at a concentration of at least 0.05, 0.1, 0.2, 0.3, 0.4, 0.5, 0.75, 1, 2, 3, 4, 5, 10, 15, 20, 25, 30, 35, 40, 45, 50, 55, 60, 65, 70, 75, 80, 85, 90, 100, 250, 500, or 750 μM. In some embodiments, vitamin K is included in the culture medium at a concentration of between 0.05 and 800 μM, between 0.05 and 750 μM, between 0.05 and 700 μM, between 0.05 and 650 μM, between 0.05 and 600 μM, between 0.05 and 550 μM, between 0.05 and 500 μM, between 0.05 and 500 μM, between 0.05 and 450 μM, between 0.05 and 400 μM, between 0.05 and 350 μM, between 0.05 and 300 μM, between 0.05 and 250 μM, between 0.05 and 200 μM, between 0.05 and 150 μM, between 0.05 and 100 μM, between 0.05 and 95 μM, between 0.05 and 90 μM, between 0.05 and 85 μM, between 0.05 and 80 μM, between 0.05 and 75 μM, between 0.05 and 70 μM, between 0.05 and 65 μM, between 0.05 and 60 μM, between 0.05 and 55 μM, between 0.05 and 50 μM, between 0.05 and 45 μM, between 0.05 and 40 μM, between 0.05 and 35 μM, between 0.05 and 30 μM, between 0.05 and 25 μM, between 0.05 and 20 μM, between 0.05 and 15 μM, between 0.05 and 10 μM, between 0.05 and 9 μM, between 0.05 and 8 μM, between 0.05 and 7 μM, between 0.05 and 6 μM, between 0.05 and 5 μM, between 0.05 and 2 μM, between 0.05 and 1 μM, between 0.05 and 0.5 μM, between 0.05 and 0.1 μM, between 0.1 and 5 μM, between 0.5 and 5 μM, or between 1 and 5 μM. In some embodiments, the cell (e.g., a differentiating hepatocyte-like progenitor cell) is subjected to vitamin K2 for at least 1, 2, 3, 4, 5, 6, 7, 8, 9, 10, 11, 12, 13, 14, 15, 16, 17, 18, 19, 20, 21, 22, 23, or 24 hours. In some embodiments, the differentiating cell is subjected to vitamin for at least 8 hours. In some embodiments, arginine is added to the culture medium together with the vitamin K2. In some embodiments, arginine is added at a concentration of 750 μM- 10 mM. In some embodiments, arginine is added to the culture medium at a similar concentration to vitamin K2. In some embodiments, the cell culture medium further includes Oncostatin-M (OSM) and/or hepatocyte growth factor (HGF). Culture Medium

Differentiation to the mature hepatocyte-like cells can occur in the presence of a culture medium that includes one or more growth factors. In some embodiments, the differentiation occurs in a culture medium that includes hepatocyte growth factor (HGF), and Oncostatin-M (OSM). In some embodiments, wherein differentiation occurs through an intermediate hepatocyte-like progenitor cell, the differentiation to the mature hepatocyte-like cell is done in the absence of HGF. In some embodiments, the culture medium includes OSM, but is HGF-free. In some embodiments, wherein differentiation occurs through an intermediate hepatocyte-like progenitor cell, the differentiation from intermediate hepatocyte-like progenitor cell to the mature hepatocyte-like cell is initially performed in the presence of HGF and the HGF is removed from the culture medium 1, 2, 3, 4, 5, 6, 7, 8, 9, 10, 11, 12, 13, 14, 15, 16, 17, 18, 19, 20, 21, 21, 22, 23, or 24 hours after the start of differentiation. In some embodiments, the HGF is removed from the culture medium 2, 3, 4, 5, 6, 7, 8, 9, or 10 days from the beginning of differentiation of the hepatocyte-like progenitor cell to the mature hepatocyte-like cell.

### Increased Oxygen Conditions

Without being bound by anything particular theory of operation and as shown in the examples provided herein, hepatocyte-like cells cultured under elevated oxygen conditions exhibit increased in vitro ureagenesis. Thus, in some embodiments, differentiation to the mature hepatocyte-like cell according to the methods provided herein occurs at an elevated oxygen level that promotes enhanced in vitro ureagenesis. In some embodiments, the differentiation occurs under normoxia (normoxic) conditions (˜20% partial pressure of O₂). In some embodiments, the differentiation occurs under hyperoxia conditions (>20% partial pressure). In some embodiments, the differentiation to mature hepatocyte-like cell is performed at above 5, 6, 7, 8, 9, 10, 11, 12, 13, 14, 15, 16, 17, 18, 19, 20, 21, 22, 23, 24, 25, 26, 27, 28, 29, 30, 35, or 40% partial pressure of O₂. In some embodiments, the differentiation to mature hepatocyte-like cell is performed at 5-30%, 6-30%, γ-30%, 8-30%, 9-30%, 10-30%, 11-30%, 12-30%, 13-30%, 14-30%, 15-30%, 16-30%, 17-30%, 18-30%, 19-30%, 20-30%, 21-30%, 22-30%, 23-30%, 24-30%, 25-30%, 26-30%, 27-30%, 28-30%, 29-30%, 15-25%, 15-35%, 15-40%, or 20-25% partial pressure of O₂.

In some embodiments, differentiation to the mature hepatocyte-like cell according to the methods provided herein occurs under at least two oxygen conditions. In some instances, one of the oxygen conditions is a hypoxic condition and the other oxygen condition is a normoxic condition. In some embodiments, the hypoxic condition comprises an oxygen content of about 0% to about 5% oxygen, e.g., about 0%-5%, about 0%-4%, about 0%-3%, about 0%-2%, about 0%-3%, about 0%-4%, about 1%-5%, about 1%-4%, about 1%-3%, about 1%-2%, about 2%-3%, about 3%-4%, about 4%-5%, about 2%-5%, and about 3%-5% oxygen content. In some embodiments, the normoxic condition comprises an oxygen content of about 20% to about 22% oxygen, e.g., about 20%-22%, about 20%-21%, about 21%-22%, about 20%, about 21%, and about 22% oxygen content. In some embodiments, the differentiating cells are exposed to a hypoxic condition for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or more days. In some embodiments, the differentiating cells are exposed to a normoxic condition for at least 1, 2, 3, 4, 5, 6, 7, 8, 9, 10, 11, 12, 13, 14, 15, 16, 17, 18, 19. 20, 21, 22, 23, 24, 25, 26, 27, 28, 29, 30, 31, 32, 33, 34, 35, 36, or more days.

### Culturing Systems

The differentiation methods described herein can be carried out with cells in suspension or attached to a 2D or 3D solid support. Solid supports include, but are not limited to, glass or plastic culture dishes, multiwell dishes, flasks, plates, microcarriers, polyvinylidene fluoride, polymer films such as metal films and 3D scaffolds. In certain embodiments, the differentiation occurs in a 2D culture system. In some embodiments, the differentiation occurs in a 3D scaffold composed of a synthetic or natural hydrogel. In exemplary embodiments, the 3D scaffold is a poly(ethylene glycol) (PEG) scaffold. In particular embodiments, the 3D scaffold is an inverted colloidal crystal PEG scaffold. See, e.g., Shirahama et al., J Vis Exp. 114:54331 (2016), which is incorporated herein by reference, particularly in parts pertinent to inverted colloidal crystal PEG scaffolds.

In some embodiments, differentiation to mature hepatocyte-like cells is carried out on a 2D or 3D solid support that includes one or more extracellular matrix components. Extracellular matrix components include, but are not limited to, type I, type II, and type IV collagen, concanavalin A, chondroitin sulfate, fibronectin, “superfibronectin” and/or fibronectin-like polymers, gelatin, laminin, poly-D and poly-L-lysine, Matrigel™, thrombospondin, and/or vitronectin. In exemplary embodiments, the solid support is coated with recombinant laminin (e.g., Laminin 521, BioLamina). In some embodiments, the solid support is free of fetal bovine serum (FBS). In some embodiments wherein differentiation occurs via a hepatocyte-like progenitor cell intermediate, the hepatocyte-like progenitor cell intermediate is transferred to a solid substrate coated with an ECM component that promotes differentiation to the mature hepatocyte-like cell. In some embodiments, the differentiation occurs initially in a fetal bovine serum free first substrate and then the differentiating cells are transferred to a second substrate that includes laminin and/or collagen to promote differentiation to the mature hepatocyte-like cell. In some embodiments, the differentiating cells are transferred to the second substrate at about day 10, 11, 12, 13, 14, 15, 16, 17, 18, 19, 20, 21, 22, 23, or 24 of differentiation. In some embodiments, the first substrate includes gelatin. In some embodiments, the second substrate further includes fetal bovine serum. Exemplary ECM component that promotes differentiation to the mature hepatocyte-like cell include, for example, recombinant Laminin 411 (BioLamina).

In some embodiments, the differentiation is carried out using a hydrogel substrate. Hydrogel substrates can be used with the differentiation methods provided as part of a 2D or 3D culturing system. In some embodiments, the hydrogel matrix has an elastic modulus of less than 2, 1, 0.5, or 0.1 kPa. In some embodiments, the hydrogel matrix has an elastic modulus of between 10 Pa and 5 kPa, between 10 Pa and 1 kPa, between 8 Pa and 1 kPa, between 50 Pa and 5 kPa, between 100 Pa and 5 kPa, between 200 Pa and 5 kPa, between 250 Pa and 5 kPa, between 500 Pa and 5 kPa, between 750 Pa and 5 kPa, 1 kPa and 5 kPa, between 10 Pa and 5 kPa, between 10 Pa and 1 kPa, between 10 Pa and 900 Pa, between 10 Pa and 800 Pa, between 10 Pa and 700 Pa, between 10 Pa and 600 Pa, between 10 Pa and 500 Pa, between 10 Pa and 400 Pa, between 10 Pa and 300 Pa, between 10 Pa and 200 Pa, between 10 Pa and 100 Pa, between 10 Pa and 50 Pa, or between 10 Pa and 25 Pa. In some embodiments, the hydrogel matrix has an elastic modulus of at least 1, 2, 3, 4, 5, 10, 15, 20, 25, 30, 35, 40, 45, 50 kPa. In some embodiments, the hydrogel matrix has an elastic modulus of between 1 and 100 kPa, between 5 and 100 kPa, between 10 and 100 kPa, between 25 and 100 kPa, between 50 and 100 kPa, between 1 and 100 kPa, between 1 and 50 kPa, between 1 and 40 kPa, between 1 and 30 kPa, between 1 and 25 kPa, between 1 and 20 kPa, between 1 and 15 kPa, between 1 and 10 kPa, between 10 and 50 kPa, between 10 and 40 kPa, between 10 and 35 kPa, between 10 and 30 kPa, between 10 and 25 kPa, between 10 and 20 kPa, between 10 and 15 kPa, between 1 and 10 kPa, between 20 and 50 kPa, between 20 and 40 kPa, between 20 and 35 kPa, between 20 and 30 kPa, between 20 and 25 kPa, between 30 and 50 kPa, between 30 and 40 kPa, between 30 and 35 kPa, or between 40 and 50 kPa. In certain embodiments, the hydrogel matrix has an elastic modulus that prevents Yes Associated Protein 1 (YAP1) from interfering with differentiation to the mature hepatocyte-like cell. It is believed that stiff matrix causes cell spreading in hepatocyte-like progenitor cell via nuclear YAP1. Such cell spreading causes the hepatocyte-like progenitor cell to differentiate into a cholangiocyte and hinders hepatocyte differentiation. Thus, in some embodiments, elastic hydrogel substrates (less than 2 kPa) advantageously promote differentiation to mature hepatocyte-like cells. In some embodiments, the hydrogel substrate is attached to one or more ECM component. In some embodiments, the ECM component is attached to the hydrogel matrix at a density of at least 50, 75, 100, 150, 200, 250, 300, 400, 450. 500, 600, 700, 800, 900 or 1,000 μM of ECM. In some embodiments, the ECM component is attached to the hydrogel matrix at a density of less than 50, 75. 100, 150, 200, 250, 300, 400, 450, 500. 600, 700, 800. 900 or 1.000 μM of ECM. In some embodiments, the ECM component is attached to the hydrogel matrix at a density of at least between 50 and 5,000 μM, between 50 and 4,000 μM between 50 and 3,000 μM, between 50 and 2,000 μM, between 50 and 1,000 μM, between 50 and 500 μM, between 50 and 400 μM, between 1 and 300 μM, between 50 and 200 μM, between 50 and 100 μM, between 50 and 75 μM, between 50 and 50 μM, between 50 and 25 μM, between 50 and 10 μM, between 100 and 5,000 μM, between 100 and 4,000 μM between 100 and 3,000 μM, between 100 and 2,000 μM, between 100 and 1,000 μM, between 100 and 1000 μM, between 100 and 400 μM, between 1 and 300 μM, between 100 and 200 μM, between 250 and 5,000 μM, between 500 and 5,000 μM, between 750 and 5,000 μM, between 1,000 and 5.000 μM, between 2,000 and 5,000 μM, between 3.000 and 5,000 μM, between 4,000 and 5,000 μM, between 250 and 5,000 μM, between 500 and 5,000 μM, between 750 and 5,000 μM, between 1,000 and 5,000 μM, between 2,000 and 5,000 μM, between 3,000 and 5,000 μM, between 4,000 and 5,000 μM, between 100 and 1,000 μM, between 200 and 900 μM, between 300 and 800 μM, between 400 and 700 μM, or between 500 and 600 μM of ECM. In some embodiments, differentiation to mature hepatocyte-like cell occurs in the presence of an elastic hydrogel with a high density of ECM component. Stiff hydrogels that include a low density of ECM components and elastic hydrogels that include a low density of ECM components promote maturation to mature hepatocyte-like cells. In some embodiments, differentiation to mature hepatocyte-like cell occurs in the presence of an elastic hydrogel that includes a high density of ECM component. In some embodiments, differentiation to mature hepatocyte-like cell occurs in the presence of a stiff hydrogel that includes a low density of ECM component.

In some embodiments, the differentiating cell is co-cultured in the presence of Human Umbilical Vein Endothelial Cells (HUVECs). It has been shown that differentiation to mature hepatocyte-like cell enhances in vitro ureagenesis capability. In some embodiments wherein differentiation occurs by a hepatocyte-like progenitor cell intermediate, the hepatocyte-like progenitor cell is cultured in the presence of the HUVECs. In some embodiments, the hepatocyte-like progenitor cell is in direct contact with the HUVECs in the culture. In other embodiments, the hepatocyte-like progenitor cell and HUVECs are cultured in two different compartments separated by a porous membrane (e.g., a transwell culture system).

### Hepatocyte Differentiation Protocol in 2D Adherent Cultures

Pluripotent stem cell-derived hepatocyte-like cells may be generated using methods using a 2D adherent culture system. The method includes single-cell passaging and xeno-free components. In some embodiments, pluripotent stem cells are maintained in standard cell culture media to maintain pluripotency prior to differentiation.

In some embodiments, the pluripotent stem cells are cultured in a media comprising one or more factors including, but not limited to, activin A, bFGF, BMP4, LY294002, CHIR99021 on day 0. In some embodiments, the pluripotent stem cells are cultured in a first media comprising activin A, bFGF, BMP4, LY294002, CHIR99021 on day 0. In some instances, the pluripotent stem cells are cultured on one or more components of the ECM, including those described herein.

In some embodiments, the differentiating cells are cultured in a media comprising one or more factors including, but not limited to, activin A, bFGF, BMP4, and LY294002 on day 1. In some embodiments, the differentiating cells are cultured in a second media comprising activin A, bFGF, BMP4, and LY294002 on day 1. In some embodiments, the cultured cells at day 1 form networks of cells.

In some embodiments, the differentiating cells are cultured in a media comprising one or more factors including, but not limited to, activin A and bFGF on day 2. In some embodiments, the differentiating cells are cultured in a third media comprising activin A and bFGF on day 2. In some instances, the media further includes a B27 supplement. In some embodiments, the cultured cells at day 2 exhibit a definitive endoderm-like cell morphology and/or activity.

In some embodiments, the differentiating cells are cultured in a media comprising one or more factors including, but not limited to, activin A on days 3-7. In some embodiments, the differentiating cells are cultured in a fourth media comprising activin A on days 3-7. In some embodiments, the cultured cells at days 2-4 exhibit a mesendoderm-like cell morphology and/or activity. In some embodiments, the cultured cells at days 5-7 exhibit a definitive endoderm-like cell morphology and/or activity. In some embodiments, the cultured cells at about days 6-7 comprise hepatoblast-like cells. In some embodiments, the cultured cells at days 6-7 exhibit a hepatoblast-like cell morphology and/or activity.

In some embodiments, the differentiating cells are cultured in a media comprising one or more factors including, but not limited to, oncostatin-M and HGF on days 8-27. In some embodiments, the differentiating cells are cultured in a fifth media comprising oncostatin-M and HGF on days 8-27. In some embodiments, the media further includes a B27 supplement. In some embodiments, the cultured cells at days 8-10 exhibit a defined cuboidal morphology. In some embodiments, the cultured cells at days 13-15 exhibit a polyhedral morphology. In some embodiments, at days 13-15 of the differentiation method, the cells exhibit a hepatic endoderm cell morphology and/or activity.

In some embodiments, the differentiating cells are cultured in a media comprising one or more factors including, but not limited to, oncostatin-M, HGF, and forskolin on days 28-35. In some embodiments, the differentiating cells are cultured in a sixth media comprising oncostatin-M, HGF, and forskolin on days 28-35. In some embodiments, the media further includes chemically defined lipids and a mixture of insulin, transferrin, and selenium.

In some embodiments, at days 20-28 the cells exhibit a hepatocyte-like cell (immature hepatocyte-like cell) morphology and/or activity. In some embodiments, at days 25-35 the cells exhibit a hepatocyte-like cell (mature hepatocyte-like cell) morphology and/or activity. In some embodiments, at days 20-35 the cells comprise hepatocyte-like cells including immature hepatocyte-like cells, mature hepatocyte-like cells, or both. In some embodiments, the cells differentiated according to the method present technology comprise at least 80%, 81, 82%, 83%, 84%, 85%, 86%, 87%, 88%, 89%, 90%, 91%, 92%,93%, 94%,95%, 96%, 97%, 98%, 99% or more immature hepatocyte-like cells. In some embodiments, the cells differentiated according to the method present technology comprise at least 80%, 81, 82%, 83%, 84%, 85%, 86%, 87%, 88%, 89%, 90%, 91%, 92%, 93%, 94%, 95%, 96%, 97%, 98%, 99% or more mature hepatocyte-like cells.

In some embodiments, the mature hepatocyte-like cells are cultured in a standard media for maintaining and/or expanding hepatocytes such as but not limited to primary hepatocytes. Any of the media compositions described herein can also contain components that are essential for maintain healthy cells including, but not limited to, non-essential amino acids, glutamine, and analogs thereof.

In some embodiments, the pluripotent stem cells are cultured in a first media comprising activin A, bFGF, BMP4, LY294002, CHIR99021 on day 0. After which in some embodiments, the cells are cultured in a second media comprising activin A, bFGF, BMP4, and LY294002 on day 1. After which in some embodiments, the differentiating cells are cultured in a third media comprising activin A and bFGF on day 2. After which in some embodiments, the differentiating cells are cultured in a fourth media comprising activin A on days 3-7. After which in some embodiments, the differentiating cells are cultured in a fifth media comprising oncostatin-M and HGF on days 8-27. After which in some embodiments the differentiating cells are cultured in a sixth media comprising oncostatin-M, HGF and forskolin on days 28-35.

In some embodiments, LY294002 is present at a concentration of about 1-20 μM, about 1-15 μM, about 1-10 μM, about 1-8 μM, about 1-5 μM, about 2-15 μM, about 2-10 μM, about 2-8 μM, about 1-5 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, or about 20 μM. In many embodiments, the media comprises LY294002 at a concentration of 10 μM.

In some embodiments, CHIR-99021 is present at a concentration of about 1-15 μM, about 1-10 μM, about 1-8 μM, about 1-5 μM, about 2-15 μM, about 2-10 μM, about 2-8 μM, about 1-5 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, or about 15 μM. In many embodiments, the media comprises CHIR-99021 at a concentration of 3 μM.

In some embodiments, activin A is present at a concentration of about 20 ng/ml-200 ng/ml, about 25 ng/ml-200 ng/ml, about 30 ng/ml-200 ng/ml, about 40 ng/ml-200 ng/ml, about 50 ng/ml-200 ng/ml, about 60 ng/ml-200 ng/ml, about 70 ng/ml-200 ng/ml, about 80 ng/ml-200 ng/ml, about 90 ng/ml-200 ng/ml, about 100 ng/ml-200 ng/ml, about 20 ng/ml-100 ng/ml, about 30 ng/ml-100 ng/ml, about 40 ng/ml-100 ng/ml, about 50 ng/ml-100 ng/ml, about 60 ng/ml-100 ng/ml, about 70 ng/ml-100 ng/ml, about 20 ng/ml, about 25 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about 100 ng/ml, about 105 ng/ml, about 110 ng/ml, about 115 ng/ml, about 120 ng/ml, about 125 ng/ml, about 130 ng/ml, about 135 ng/ml, about 140 ng/ml, about 145 ng/ml, about 150 ng/ml, about 155 ng/ml, about 160 ng/ml, about 165 ng/ml, about 170 ng/ml, about 175 ng/ml, about 180 ng/ml, about 185 ng/ml, about 190 ng/ml, about 195 ng/ml, or about 200 ng/ml. In many embodiments, the media comprises activin A at a concentration of 50 ng/ml or 100 ng/ml.

In some embodiments, bFGF is present at a concentration of about 5 ng/ml, about 7 ng/ml, about 8 ng/ml, about 10 ng/ml, about 13 ng/ml, about 15 ng/ml, about 17 ng/ml, about 18 ng/ml, about 20 ng/ml, about 23 ng/ml, about 25 ng/ml, about 27 ng/ml, about 28 ng/ml, about 30 ng/ml, about 35 ng/ml, about 37 ng/ml, about 38 ng/ml, about 40 ng/ml, about 43 ng/ml, about 45 ng/ml, about 47 ng/ml, about 48 ng/ml, about 50 ng/ml, about 51 ng/ml, about 52 ng/ml, about 53 ng/ml, about 55 ng/ml, about 58 ng/ml, about 5 ng/ml-40 ng/ml, about 5 ng/ml-30 ng/ml, about 10 ng/ml-40 ng/ml, about 5 ng/ml-20 ng/ml, or about 10 ng/ml-50 ng/ml. In some embodiments, the media comprises bFGF at a concentration of 80 ng/ml

In some embodiments, BMP4 is present at a concentration of about 5 ng/ml, about 7 ng/ml, about 8 ng/ml, about 10 ng/ml, about 13 ng/ml, about 15 ng/ml, about 17 ng/ml, about 18 ng/ml, about 20 ng/ml, about 23 ng/ml, about 25 ng/ml, about 27 ng/ml, about 28 ng/ml, about 30 ng/ml, about 35 ng/ml, about 37 ng/ml, about 38 ng/ml, about 40 ng/ml, about 43 ng/ml, about 45 ng/ml, about 47 ng/ml, about 48 ng/ml, about 50 ng/ml, about 51 ng/ml, about 52 ng/ml, about 53 ng/ml, about 55 ng/ml, about 58 ng/ml, about 5 ng/ml-40 ng/ml, about 5 ng/ml-30 ng/ml, about 10 ng/ml-40 ng/ml, about 5 ng/ml-20 ng/ml, or about 10 ng/ml-50 ng/ml. In some embodiments, the media comprises BMP4 at a concentration of 10 ng/ml.

In some embodiments, OSM is present at a concentration of about 5 ng/ml, about 7 ng/ml, about 8 ng/ml, about 10 ng/ml, about 13 ng/ml, about 15 ng/ml, about 17 ng/ml, about 18 ng/ml, about 20 ng/ml, about 23 ng/ml, about 25 ng/ml, about 27 ng/ml, about 28 ng/ml, about 30 ng/ml, about 35 ng/ml, about 37 ng/ml, about 38 ng/ml, about 40 ng/ml, about 43 ng/ml, about 45 ng/ml, about 47 ng/ml, about 48 ng/ml, about 50 ng/ml, about 51 ng/ml, about 52 ng/ml, about 53 ng/ml, about 55 ng/ml, about 58 ng/ml, about 5 ng/ml-50 ng/ml, about 5 ng/ml-40 ng/ml, about 5 ng/ml-30 ng/ml, about 30 ng/ml-40 ng/ml, about 5 ng/ml-20 ng/ml, or about 30 ng/ml-50 ng/ml. In some embodiments, the media comprises OSM at a concentration of 10 ng/ml.

In some embodiments, HGF is present at a concentration of about 25 ng/ml-200 ng/ml, about 30 ng/ml-200 ng/ml, about 40 ng/ml-200 ng/ml, about 50 ng/ml-200 ng/ml, about 60 ng/ml-200 ng/ml, about 70 ng/ml-200 ng/ml, about 80 ng/ml-200 ng/ml, about 90 ng/ml-200 ng/ml, about 100 ng/ml-200 ng/ml, about 30 ng/ml-100 ng/ml, about 40 ng/ml-100 ng/ml, about 50 ng/ml-100 ng/ml, about 60 ng/ml-100 ng/ml, about 70 ng/ml-100 ng/ml, about 25 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about 100 ng/ml, about 105 ng/ml, about 110 ng/ml, about 115 ng/ml, about 120 ng/ml, about 125 ng/ml, about 130 ng/ml, about 135 ng/ml, about 140 ng/ml, about 145 ng/ml, about 150 ng/ml, about 155 ng/ml, about 160 ng/ml, about 165 ng/ml, about 170 ng/ml, about 175 ng/ml, about 180 ng/ml, about 185 ng/ml, about 190 ng/ml, about 195 ng/ml, or about 200 ng/ml. In some embodiments, the media comprises HGF at a concentration of 50 ng/ml.

In some embodiments, forskolin is present at a concentration of about 1-20 μM, about 1-15 μM, about 1-10 μM, about 1-8 μM, about 1-5 μM, about 2-15 μM, about 2-10 μM, about 2-8 μM, about 1-5 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, or about 20 μM. In some embodiments, the media comprises forskolin at a concentration of 10 μM.

### Hepatocyte Differentiation Protocol in 3D Suspension Cultures

Pluripotent stem cell-derived hepatocyte-like cells may be generated using a method using a 3D suspension culture system. In some embodiments, pluripotent stem cells are maintained in standard cell culture media to maintain pluripotency prior to differentiation.

In some embodiments, the cells are cultured in a media comprising one or more factors including, but not limited to, insulin, PIK-90, activin A, bFGF. BMP4, and CHIR99021 on day 0. In some embodiments, the pluripotent stem cells are cultured in a first media comprising insulin. PIK-90, activin A, bFGF, BMP4, and CHIR99021 on day 0. In some embodiments, the pluripotent stem cells are cultured on a solid support that facilitate low or no attachment to the support surface, including those described herein. In some embodiments, the media further includes one or more of the following components: human serum albumin, chemically defined lipids, non-essential amino acids, ascorbic acid-2-phosphate, holo-transferrin, sodium selenite, and any additional reagent for facilitating hepatocyte differentiation and/or cell health.

In some embodiments, the cells are cultured at a cell concentration (cell density) of about 500,000 to about 5,000,000 cells per mL. e.g., about 500,000-5,000,000: about 500,000-4,500,000; about 500,000-4,000,000; about 500,000-3,500,000; about 500,000-3,000,000; about 500,000-2,500,000: about 500,000-2,000,000; about 500,000-2,500,000; about 500,000-1,500.000; about 500,000-1,000.000: about 1,000,000-5,000,000: about 1,000,000-4,500,000: about 1,000,000-4,000,000; about 1,000,000-3,500,000; about 1,000,000-3,000,000; about 1,000,000-2,500,000; about 1,000,000-2,000,000; about 1,000,000-1,500,000 cells per mL,

In some embodiments, the cells are cultured under a hypoxic condition, In some embodiments, the hypoxic condition comprises an oxygen content of about 0% to about 5% oxygen, e,g,, about 0%-5%, about 0%-4%, about 0%-3%, about 0%-2%, about 0%-3%, about 0%-4%, about 1%-5%, about 1%-4%, about 1%-3%, about 1%-2%, about 2%-3%, about 3%-4%, about 4%-5%, about 2%-5%, and about 3%-5% oxygen content, In some embodiments, the cells from into aggregates,

In some embodiments, the cell aggregates are between about 100-250 μm in diameter, e,g,, about 100-250, about 100-240, about 100-230, about 100-220, about 100-210, about 100-200, about 110-250, about 120-250, about 130-250, about 140-250, about 150-250, about 160-250, about 100-200, about 100-190, about 100-180, about 100-170, about 140-250, about 140-250, about 140-240, about 140-230, about 140-220, about 140-210, about 140-200, about 140-190 μm in diameter,

In some embodiments, the differentiating cell aggregates are cultured in a media comprising one or more factors including, but not limited to, insulin, PIK-90, activin A, bFGF, and BMP4 on day 1, In some embodiments, the cell aggregates are cultured in a second media comprising insulin, PIK-90, activin A, bFGF, and BMP4 on day 1, In some embodiments, the media further includes one or more of the following components: human serum albumin, chemically defined lipids, non-essential amino acids, ascorbic acid-2-phosphate, holo-transferrin, sodium selenite, and any additional reagent for facilitating hepatocyte differentiation and/or cell health, In some embodiments, the cells outlined are cultured under a hypoxic condition from days 0-21,

In some embodiments, the differentiating cell aggregates are cultured in a media comprising one or more factors including, but not limited to, insulin, PIK-90, activin A, and bFGF on day 2, In some embodiments, the cell aggregates are cultured in a third media comprising insulin, PIK-90, activin A, and bFGF on day 2, In some embodiments, the media further includes one or more of the following components: human serum albumin, chemically defined lipids, non-essential amino acids, ascorbic acid-2-phosphate, holo-transferrin, sodium selenite, and any additional reagent for facilitating hepatocyte differentiation and/or cell health,

In some embodiments, the differentiating cell aggregates are cultured in a media comprising one or more factors including, but not limited to, B27 supplement and activin A on days 3-5, In some embodiments, the cell aggregates are cultured in a fourth media comprising B27 supplement and activin A on days 3-5, In some embodiments, the media further includes some embodiments, the media also includes one or more of the following components: human serum albumin, chemically defined lipids, non-essential amino acids, ascorbic acid-2-phosphate, holo-transferrin, sodium selenite, and any additional reagent for facilitating hepatocyte differentiation and/or cell health, The media can be refreshed, changed, or replaced daily, every other day, or as needed,

In some embodiments, the differentiating cell aggregates are cultured in a media comprising one or more factors including, but not limited to, B27 supplement, BMP4, and FGF10 on days 6-9, In some embodiments, the cell aggregates are cultured in a fifth media comprising B27 supplement, BMP4, and FGF10 on days 6-9, In some embodiments, the media further includes one or more of the following components: human serum albumin, chemically defined lipids, non-essential amino acids, ascorbic acid-2-phosphate, holo-transferrin, sodium selenite, and any additional reagent for facilitating hepatocyte differentiation and/or cell health, The media can be refreshed, changed, or replaced daily, every other day, or as needed,

In some embodiments, the differentiating cell aggregates are cultured in a media comprising one or more factors including, but not limited to, HGF on days 10-14, In some embodiments, the cell aggregates are cultured in a sixth media comprising HGF on days 10-14, In some embodiments, the media further includes chemically defined lipids and a mixture of insulin, transferrin, and selenium, The media can be refreshed, changed, or replaced daily, every other day, or as needed,

In some embodiments, the differentiating cells are cultured in a media comprising one or more factors including, but not limited to, oncostatin-M on days 15-27, In some embodiments, the cell aggregates are cultured in a seventh media comprising oncostatin-M on days 15-27, In some embodiments, the media at this stage of differentiation is free of HGF, In some embodiments, the media further includes chemically defined lipids and a mixture of insulin, transferrin, and selenium, The media can be refreshed, changed, or replaced daily, every other day, or as needed,

In some embodiments, the differentiating cells are cultured in a media comprising one or more factors including, but not limited to, oncostatin-M and forskolin on days 28-35, In many embodiments, the cell aggregates are cultured in an eighth media comprising oncostatin-M and forskolin on days 28-35, In some embodiments, the media at this stage of differentiation is free of HGF, In some embodiments, the media further includes chemically defined lipids and a mixture of insulin, transferrin, and selenium, The media can be refreshed, changed, or replaced daily, every other day, or as needed,

In some embodiments, at days 15-35 the cells comprise hepatocyte-like cells including immature hepatocyte-like cells, mature hepatocyte-like cells, or both, In some embodiments, the cells differentiated according to the method present technology of the present technology comprise at least 80%, 81, 82%, 83%, 84%, 85%, 86%, 87%, 88%, 89%, 90%, 91%, 92%, 93%, 94%, 95%, 96%, 97%, 98%, 99% or more immature hepatocyte-like cells, In some embodiments, the cells differentiated according to the method present technology comprise at least 80%, 81, 82%, 83%, 84%, 85%, 86%, 87%, 88%, 89%, 90%, 91%, 92%, 93%, 94%, 95%, 96%, 97%, 98%, 99% or more mature hepatocyte-like cells,

In some embodiments, the cell aggregates are cultured under a normoxic condition (normoxia), In some embodiments, the normoxic condition comprises an oxygen content of about 20% to about 22% oxygen, e,g,, about 20%-22%, about 20%-21%, about 21%-22%, about 20%, about 21%, and about 22% oxygen content, In other words, normoxic condition includes ambient levels of O₂, In some embodiments, the cell aggregates are cultured under a normoxic condition at at least days 15-27, 21-27, 20-28, 20-35, 27-35, 28-35, 29-35, or 30-35,

In some embodiments, the pluripotent stem cells are cultured in a first media comprising insulin, PIK-90, activin A, bFGF, BMP4, and CHIR99021 on day 0, On day 0 the cells from aggregates (also referred to as spheroids), After which in some embodiments, the cell aggregates are cultured in a second media comprising insulin, PIK-90, activin A, bFGF, and BMP4 on day 1, After which in some embodiments, the cell aggregates are cultured in a third media comprising insulin, PIK-90, activin A, and bFGF on day 2, After which in some embodiments, the cell aggregates are cultured in a fourth media comprising B27 supplement and activin A on days 3-5, After which in some embodiments, the cell aggregates are cultured in a fifth media comprising B27 supplement, BMP4, and FGF10 on days 6-9, After which in some embodiments, the cell aggregates are cultured in a sixth media comprising HGF on days 10-14, After which in some embodiments, the cell aggregates are cultured in a seventh media comprising oncostatin-M on days 15-27, After which in some embodiments, the cell aggregates are cultured in an eighth media comprising oncostatin-M and forskolin on days 28-35,

In some embodiments, the differentiating cells exhibit definite endoderm cell characteristics at days 0-2, In some embodiments, the differentiating cells exhibit anterior definite endoderm cell characteristics at days 3-5, In some embodiments, the differentiating cells exhibit foregut endoderm cell characteristics at days 6-9, In some embodiments, the differentiating cells exhibit hepatic endoderm cell characteristics at days 10-14, In some embodiments, the differentiating cells exhibit immature hepatocyte-like cell characteristics at days 15-35,

In some embodiments, insulin is present at a concentration of about 0,1 μg/ml, about 0,2 μg/ml, about 0,3 μg/ml, about 0,4 μg/ml, about 0,5 μg/ml, about 0,6 μg/ml, about 0,1 μg/ml-about 0,6 μg/ml, about 0,1 μg/ml-about 0,5 μg/ml, about 0,1 μg/ml-about 0,4 μg/ml, about 0,2 μg/ml-about 0,6 μg/ml, about 0,2 μg/ml-about 0,5 μg/ml, or about 0,2 μg/ml-about 0,4 μg/ml, In some embodiments, the media comprises insulin at a concentration of 0,2 μg/ml,

In some embodiments, PIK-90 is present at a concentration of about 0,1 μM, about 0,2 μM, about 0,3 μM, about 0,4 μM, about 0,5 μM, about 0,6 μM, about 0,1 μM-about 0,6 μM, about 0,1 μM-about 0,5 μM, about 0,1 μM-about 0,4 μM, about 0,2 μM-about 0,6 μM, about 0,2 μM-about 0,5 μM, or about 0,2 μM about 0,4 μM, In some embodiments, the media comprises PIK-90 at a concentration of 0,1 M,

In some embodiments, CHIR-99021 is present at a concentration of about 1-15 μM, about 1-10 μM, about 1-8 μM, about 1-5 μM, about 2-15 μM, about 2-10 μM, about 2-8 μM, about 1-5 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, or about 15 μM, In some embodiments, the media comprises CHIR-99021 at a concentration of 3 μM,

In some embodiments, activin A is present at a concentration of about 20 ng/ml-200 ng/ml, about 25 ng/ml-200 ng/ml, about 30 ng/ml-200 ng/ml, about 40 ng/ml-200 ng/ml, about 50 ng/ml-200 ng/ml, about 60 ng/mi-200 ng/ml, about 70 ng/ml-200 ng/ml, about 80 ng/ml-200 ng/ml, about 90 ng/ml-200 ng/ml, about 100 ng/ml-200 ng/ml, about 20 ng/ml-100 ng/ml, about 30 ng/ml-100 ng/ml, about 40 ng/ml-100 ng/ml, about 50 ng/ml-100 ng/ml, about 60 ng/ml-100 ng/ml, about 70 ng/mi-100 ng/ml, about 20 ng/ml, about 25 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about 100 ng/ml, about 105 ng/ml, about 110 ng/ml, about 115 ng/ml, about 120 ng/ml, about 125 ng/ml, about 130 ng/ml, about 135 ng/ml, about 140 ng/ml, about 145 ng/ml, about 150 ng/ml, about 155 ng/ml, about 160 ng/ml, about 165 ng/ml, about 170 ng/ml, about 175 ng/ml, about 180 ng/ml, about 185 ng/ml, about 190 ng/ml, about 195 ng/ml, or about 200 ng/ml, In some embodiments, the media comprises activin A at a concentration of 50 ng/ml or 100 ng/ml,

In some embodiments, bFGF is present at a concentration of about 5 ng/ml, about 7 ng/ml, about 8 ng/ml, about 10 ng/ml, about 13 ng/ml, about 15 ng/ml, about 17 ng/ml, about 18 ng/ml, about 20 ng/ml, about 23 ng/ml, about 25 ng/ml, about 27 ng/ml, about 28 ng/ml, about 30 ng/ml, about 35 ng/ml, about 37 ng/ml, about 38 ng/ml, about 40 ng/ml, about 43 ng/ml, about 45 ng/ml, about 47 ng/ml, about 48 ng/ml, about 50 ng/ml, about 51 ng/ml, about 52 ng/ml, about 53 ng/ml, about 55 ng/ml, about 58 ng/ml, about 5 ng/ml-40 ng/ml, about 5 ng/ml-30 ng/ml, about 10 ng/ml-40 ng/ml, about 5 ng/ml-20 ng/ml, or about 10 ng/ml-50 ng/ml, In some embodiments, the media comprises bFGF at a concentration of 10 ng/ml or 40 ng/ml,

In some embodiments, BMP4 is present at a concentration of about 5 ng/ml, about 7 ng/ml, about 8 ng/ml, about 10 ng/ml, about 13 ng/ml, about 15 ng/ml, about 17 ng/ml, about 18 ng/ml, about 20 ng/ml, about 23 ng/ml, about 25 ng/ml, about 27 ng/ml, about 28 ng/ml, about 30 ng/ml, about 35 ng/ml, about 37 ng/ml, about 38 ng/ml, about 40 ng/ml, about 43 ng/ml, about 45 ng/ml, about 47 ng/ml, about 48 ng/ml, about 50 ng/ml, about 51 ng/ml, about 52 ng/ml, about 53 ng/ml, about 55 ng/ml, about 58 ng/ml, about 5 ng/ml-40 ng/ml, about 5 ng/ml-30 ng/mil, about 10 ng/ml-40 ng/mil, about 5 ng/ml-20 ng/ml, or about 10 ng/ml-50 ng/ml, In some embodiments, the media comprises BMP4 at a concentration of 10 ng/ml or 20 ng/ml,

In some embodiments, OSM is present at a concentration of about 5 ng/ml, about 7 ng/ml, about 8 ng/ml, about 10 ng/ml, about 13 ng/ml, about 15 ng/ml, about 17 ng/ml, about 18 ng/ml, about 20 ng/ml, about 23 ng/ml, about 25 ng/ml, about 27 ng/ml, about 28 ng/ml, about 30 ng/ml, about 35 ng/ml, about 37 ng/ml, about 38 ng/ml, about 40 ng/ml, about 43 ng/ml, about 45 ng/ml, about 47 ng/ml, about 48 ng/ml, about 50 ng/ml, about 51 ng/ml, about 52 ng/ml, about 53 ng/ml, about 55 ng/ml, about 58 ng/ml, about 5 ng/ml-50 ng/ml, about 5 ng/ml-40 ng/ml, about 5 ng/ml-30 ng/ml, about 30 ng/ml-40 ng/ml, about 5 ng/ml-20 ng/ml, or about 30 ng/mi-50 ng/ml, In some embodiments, the media comprises OSM at a concentration of 30 ng/ml,

In some embodiments, HGF is present at a concentration of about 25 ng/ml-200 ng/ml, about 30 ng/ml-200 ng/ml, about 40 ng/m-200 ng/ml, about 50 ng/mi-200 ng/ml, about 60 ng/ml-200 ng/ml, about 70 ng/ml-200 ng/ml, about 80 ng/ml-200 ng/ml, about 90 ng/ml-200 ng/ml, about 100 ng/ml-200 ng/ml, about 30 ng/ml-100 ng/ml, about 40 ng/ml-100 ng/ml, about 50 ng/ml-100 ng/ml, about 60 ng/ml-100 ng/ml, about 70 ng/ml-100 ng/ml, about 25 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about 100 ng/ml, about 105 ng/ml, about 110 ng/ml, about 115 ng/ml, about 120 ng/ml, about 125 ng/ml, about 130 ng/ml, about 135 ng/ml, about 140 ng/ml, about 145 ng/ml, about 150 ng/ml, about 155 ng/ml, about 160 ng/ml, about 165 ng/ml, about 170 ng/ml, about 175 ng/ml, about 180 ng/ml, about 185 ng/ml, about 190 ng/ml, about 195 ng/ml, or about 200 ng/ml, In some embodiments, the media comprises HGF at a concentration of 50 ng/ml,

In some embodiments, forskolin is present at a concentration of about 1-20 μM, about 1-15 μM, about 1-10 μM, about 1-8 μM, about 1-5 μM, about 2-15 μM, about 2-10 μM, about 2-8 μM, about 1-5 μM, about 1 μM, about 2 μM, about 3 μM, about 4 μM, about 5 IM, about 6 μM, about 7 μM, about 8 μM, about 9 μM, about 10 μM, about 11 μM, about 12 PM, about 13 μM, about 14 μM, about 15 μM, about 16 μM, about 17 μM, about 18 M, about 19 μM, or about 20 μM, In some embodiments, the media comprises forskolin at a concentration of 10 μM,

Hepatocytes as described herein may be used for treating diseases or conditions associated with liver dysfunction,

Methods of treating a subject having liver disease are described, In some embodiments, the method comprises administering a liver stem cell described herein to the subject, such as by contacting the liver stem cell with the liver of the subject in vivo, or by transplanting a liver stem cell into the liver of the subject, wherein the administered or transplanted cell integrates into and repopulates the liver of the subject, thereby treating the liver disease, In some embodiments, the method comprises administering a differentiated liver stem cell or stem cell derived cell described herein to the subject, such as by contacting the differentiated liver stem cell or stem cell derived cell with the liver of the subject in vivo, or by transplanting a liver stem cell or stem cell derived cell into the liver of the subject, wherein the administered or transplanted cell integrates into and repopulates the liver of the subject, thereby treating the liver disease, In some embodiments, the liver disease is selected from the group of metabolic disease, autoimmune disease, infectious disease, drug induced acute and chronic liver failure, cirrhosis, and liver cancer,

For example, hepatocytes described herein may be used for treating liver cirrhosis, Such hepatocyptes may therefore be used for liver regeneration in patients with liver cirrhosis,

The liver stem cell (LSC) and their differentiated hepatocyte (dHep) and bile duct cells have the potential to treat liver cirrhosis caused by different liver diseases, In vitro derived autologous LSC, dHep and bile duct cells can be delivered to the same patient to rescue liver failure and reduce liver cirrhosis, LSC can be derived from the same patient and in vitro cultured for expansion, dHep and bile duct cells are in vitro differentiated from LSC, These stem cells and differentiated cells are autologous and will not induce transplant rejection in patients,

The liver stem cell (LSC) and their differentiated hepatocyte (dHep) and bile duct cells have the potential to treat liver cirrhosis caused by different liver diseases, In vitro derived allogeneic LSC, dHep and bile duct cells can be delivered to a patient to rescue liver failure and reduce liver cirrhosis, LSC can be derived from a donor and in vitro cultured for expansion, dHep and bile duct cells can be in vitro differentiated from LSC, These stem cells and differentiated cells are allogeneic and, where subjected to some or all of the hypoimmune modifications described herein, will not induce transplant rejection in patients,

WO2016200340A1 demonstrates that both liver stem cells and differentiated liver stem cells (dHep) rescued the liver cirrhosis phenotype when transplanted into a mice treated with thioacetamide to induce liver cirrhosis mice, The mice developed liver cirrhosis by continuing thioacetamide treatment for 2-3 months, As described in WO2016200340A1, liver stem cells and liver stem cell differentiated dHep cells may be transplanted into the mice by intrasplenic injection, After transplantation, the mice may continue to receive drug treatment for another 3 months, Mouse liver functions may be tested in the third month, Transplanted mice group may have higher serum albumin than non-transplanted control group, Their liver functions therefore may be partially rescued, The livers of mice receiving transplanted dHEP cells may have less fibrosis and inflammation than control group, dHep may be repopulated in transplanted mouse liver, Both liver stem cells and dHEP cells may integrate into mouse liver tissue and express human specific albumin,

Diseases and disorders that may be treated using the hepatocytes described herein include but are not limited to Crigler-Najjar syndrome type 1; familial hypercholesterolemia; Factor VII deficiency; Glycogen storage disease type I; infantile Refsum's disease; Progressive familial intrahepatic cholestasis type 2; hereditary tyrosinemia type 1; and various urea cycle defects; acute liver failure, including juvenile and adult patients with acute drug-induced liver failure; viral-induced acute liver failure; idiopathic acute liver failure: mushroom-poisoning-induced acute liver failure; post-surgery acute liver failure; acute liver failure induced by acute fatty liver of pregnancy; chronic liver disease, including cirrhosis and/or fibrosis: acute-on-chronic liver disease caused by one of the following acute events: alcohol consumption, drug ingestion, and/or hepatitis B flares, Thus, the patients may have one or more of these or other liver conditions,

Diseases and disorders that may be treated using the hepatocytes described herein include hepatocyte-specific (hepatocyte-intrinsic) dysfunction, For example, the dysfunction, and the etiology of the disease and/or disorder, may be due to, or primarily attributable to, dysfunction of the endogenous hepatocytes present within the subject, In some embodiments, the hepatocyte-specific dysfunction may be genetic or inherited by the subject, In some embodiments, the etiology of the disease or disorder does not substantially involve cell types other than hepatocytes, In some embodiments, the disease or disorder results in decreased liver function, liver disease (acute or chronic), or other adverse condition derived from the endogenous hepatocytes, Accordingly, in some embodiments, e,g,, where a disease is intrinsic to the endogenous hepatocyte population, an effective treatment may include replacement, supplementation, transplantation, or repopulation with hepatocytes as described herein, Without being bound by theory, in hepatocyte-intrinsic diseases/disorders replacement and/or supplementation of the endogenous hepatocytes can result in significant clinical improvement without the disease/disorder negatively impacting the transplanted hepatocytes, For example, where a subject has a genetic disorder affecting hepatocyte function (e,g,, amino acid metabolism within hepatocytes, such as e,g,, a hypertyrosinemia) allogenic transplanted hepatocytes may be essentially unaffected by the presence of the disease/disorder within the subject, Thus, transplanted hepatocytes may substantially engraft, survive, expand, and/or repopulate within the subject, resulting in a significant positive clinical outcome,

Diseases and disorders characterized by hepatocyte- specific (hepatocyte-intrinsic) dysfunction may be contrasted with diseases and disorders having an etiology that is not hepatocyte specific and involve hepatocyte extrinsic factors, Examples of diseases having factors and/or an etiology that is hepatocyte extrinsic include but are not limited to e,g,, alcoholic steatohepatitis, alcoholic liver disease (ALD), hepatic steatosis/nonalcoholic fatty liver disease (NAFLD), and the like, Hepatocyte extrinsic diseases involve hepatic insults that are external, or derived from outside the endogenous hepatocytes, such as alcohol, diet, infection, etc, In some embodiments, diseases and disorders treated according to the methods described herein may include diseases and disorders that are not hepatocyte-specific (hepatocyte-intrinsic) dysfunction,

Examples of hepatocyte-intrinsic and hepatocyte-related diseases include liver-related enzyme deficiencies, hepatocyte-related transport diseases, and the like, Such liver-related deficiencies may be acquired or inherited diseases and may include metabolic diseases (such as e,g, liver-based metabolic disorders), Inherited liver-based metabolic disorders may be referred to “inherited metabolic diseases of the liver”, such as but not limited to e,g,, those diseases described in Ishak, Clin Liver Dis (2002) 6:455-479, Liver-related deficiencies may, in some instances, result in acute and/or chronic liver disease, including e,g,, where acute and/or chronic liver disease is a result of the deficiency when left untreated or insufficiently treated, Non-limiting examples of inherited liver-related enzyme deficiencies, hepatocyte-related transport diseases, and the like include Crigler-Najjar syndrome type 1; familial hypercholesterolemia, Factor VII deficiency, Glycogen storage disease type I, infantile Refsum's disease, Progressive familial intrahepatic cholestasis type 2, hereditary tyrosinemias (e,g,, hereditary tyrosinemia type 1), genetic urea cycle defects, phenylketonuria (PKU), hereditary hemochromatosis, Alpha-I antitrypsin deficiency (AATD), Wilson Disease, and the like, Non-limiting examples of inherited metabolic diseases of the liver, including metabolic diseases having at least some liver phenotype, pathology, and/or liver-related symptom(s), include 5-beta-reductase deficiency, AACT deficiency, Aarskog syndrome, abetalipoproteinemia, adrenal leukodystrophy, Alpers disease, Alpers syndrome, alpha-1-antitrypsin deficiency, antithrombin III deficiency, arginase deficiency, argininosuccinic aciduria, arteriohepatic dysplasia, autoimmune lymphoproliferative syndrome, benign recurrent cholestasis, beta-thalassemia, Bloom syndrome, Budd-Chiari syndrome, carbohydrate-deficient glycoprotein syndrome, ceramidase deficiency, ceroid lipofuscinosis, cholesterol ester storage disease, cholesteryl ester storage disease, chronic granulomatous, chronic hepatitis C, Crigler- Najjar syndrome, cystic fibrosis, cystinosis, diabetes mellitus, Dubin-Johnson syndrome, endemic Tyrolean cirrhosis, erythropoietic protoporphyria, Fabry disease, familial hypercholesterolemia, familial steatohepatitis, fibrinogen storage disease, galactosemia, gangliosidosis, Gaucher disease, genetic hemochromatosis, glycogenosis type 1a, glycogenosis type 2, glycogenosis type 3, glycogenosis type 4, granulomatous disease, hepatic familial amyloidosis, hereditary fructose intolerance, hereditary spherocytosis, Hermansky-Pudlak syndrome, homocystinuria, hyperoxaluria, hypobetalipoproteinemia, hypofibrinogenemia, intrahepatic cholestasis of pregnancy, Lafora disease, lipoamide dehydrogenase deficiency, lipoprotein disorders, Mauriac syndrome, metachromatic leukodystrophy, mitochondrial cytopathies, Navajo neurohepatopathy, Niemann-Pick disease, nonsyndromic paucity of bile ducts, North American Indian childhood cirrhosis, omithine transcarbamylase deficiency, partial lipodystrophy, Pearson syndrome, porphyria cutanea tarda, progressive familial intrahepatic cholestasis, progressive familial intrahepatic cholestasis type 1, progressive familial intrahepatic cholestasis type 2, protein C deficiency, Shwachman syndrome, Tangier disease, thrombocytopenic purpura, total lipodystrophy, type 1 glycogenosis, Tyrolean cirrhosis, tyrosinemia, urea cycle disorders, venocclusive disease, Wilson disease, Wolman disease, X-linked hyper-IgM syndrome, and Zellweger syndrome,

Treatment of subjects according to the methods described herein may result in various clinical benefits and/or measurable outcomes, including but not limited to e,g,, prolonged survival, delayed disease progression (e,g,, delayed liver failure), prevention of liver failure, improved and/or normalized liver function, improved and/or normalized amino acid levels, improved and/or normalized ammonia levels, improved and/or normalized albumin levels, improved and/or normalized bilirubin, recovery from a failure to thrive phenotype, reduction in lethargy, reduction in obtundation, reduction in seizures, reduction in jaundice, improved and/or normalized serum glucose, improved and/or normalized INR, improved and/or normalized urine test results, and the like,

For example, in some instances, administration of genetically modified hepatocytes and/or hepatocyte progenitors as described herein results in at least a 5% increase in survival of subjects having a liver disease and/or a condition resulting in liver failure as compared to e,g,, subjects treated according to the standard of care and/or administered hepatocytes and/or hepatocyte progenitors that have not been genetically modified as described herein, The observed level of enhanced survival in such subject may vary and may range from an at least 5% to 60% or more increase, including but not limited to e,g,, an at least 5%, at least 10%, at least 15%, at least 20%, at least 25%, at least 30%, at least 35%, at least 40%, at least 45%, at least 50%, at least 55%, at least 60% or more increase in survival, In some embodiments, subjects administered genetically modified hepatocytes and/or progenitors thereof as described herein may experience a delay in disease progression and/or the onset of one or more disease symptoms, such as but not limited to e,g,, liver failure and/or any symptom(s) attributable thereto, Such a delay in disease progression and/or symptom onset may last days, weeks, months or years, including but not limited to e,g,, at least one week, at least one month, at least 2 months, at least 3 months, at least 4 months, at least 5 months, at least 6 months, at least a year or more, The hepatocytes as described herein administered to a patient effect a beneficial therapeutic response in the patient over time,

Non-limiting examples of liver conditions that may be treated include acute intermittent porphyria, acute liver failure, alagille syndrome, alcoholic fatty liver disease, alcoholic hepatitis, alcoholic liver cirrhosis, alcoholic liver disease, alpha 1-antitrypsin deficiency, amebic liver abscess, autoimmune hepatitis, biliary liver cirrhosis, budd-chiari syndrome, chemical and drug induced liver injury, cholestasis, chronic hepatitis, chronic hepatitis B, chronic hepatitis C, chronic hepatitis D, end stage liver disease, erythropoietic protoporphyria, fascioliasis, fatty liver disease, focal nodular hyperplasia, hepatic echinococcosis, hepatic encephalopathy, hepatic infarction, hepatic insufficiency, hepatic porphyrias, hepatic tuberculosis, hepatic veno-occlusive disease, hepatitis, hepatocellular carcinoma, hepatoerythropoietic porphyria, hepatolenticular degeneration, hepatomegaly, hepatopulmonary syndrome, hepatorenal syndrome, hereditary coproporphyria, liver abscess, liver cell adenoma, liver cirrhosis, liver failure, liver neoplasm, massive hepatic necrosis, nonalcoholic fatty liver disease, parasitic liver disease, peliosis hepatis, porphyria cutanea tarda, portal hypertension, pyogenic liver abscess, reye syndrome, variegate porphyria, viral hepatitis, viral hepatitis A, viral hepatitis B, viral hepatitis C, viral hepatitis D, viral hepatitis E, and zellweger syndrome, and the like, In some instances, a subject may be treated for fibrosis or a fibrotic condition, In some instances, a subject may be treated for cirrhosis or a cirrhotic condition,

Treatments described herein may be performed chronically (i,e,, continuously) or non-chronically (i,e,, non-continuously) and may include administration of one or more agents chronically (i,e,, continuously) or non-chronically (i,e,, non-continuously), Chronic administration of one or more agents according to the methods described herein may be employed in various instances, including e,g,, where a subject has a chronic condition, including e,g,, a chronic liver condition (e,g,, chronic liver disease, cirrhosis, alcoholic liver disease, nonalcoholic fatty liver disease (NAFLD/NASH), chronic viral hepatitis, etc,), a chronic genetic liver condition (alpha-1 antitrypsin deficiency, Hereditary hemochromatosis, Wilson disease, etc,), chronic liver-related autoimmune conditions (e,g,, primary biliary cirrhosis (PBC), primary sclerosing cholangitis (PSC), autoimmune hepatitis (AIH), etc,) etc, Administration of one or more agents for a chronic condition may include but is not limited to administration of the agent for multiple months, a year or more, multiple years, etc, Such chronic administration may be performed at any convenient and appropriate dosing schedule including but not limited to e,g,, daily, twice daily, weekly, twice weekly, monthly, twice monthly, etc, In some instances, e,g,, in the case of correction of a genetic condition or other persistent gene therapies, a chronic condition may be treated by a single or few (e,g,, 2, 3, 4, or 5) treatments, Non-chronic administration of one or more agents may include but is not limited to e,g,, administration for a month or less, including e,g,, a period of weeks, a week, a period of days, a limited number of doses (e,g,, less than 10 doses, e,g,, 9 doses or less, 8 doses or less, 7 doses or less, etc,, including a single dose),

In some embodiments, the amount of genetically modified hepatocytes administered to a subject may include e,g,, at least 10 million, at least 25 million, at least 50 million, at least 75 million, at least 100 million, at least 250 million, at least 500 million, at least 750 million, at least 1 billion, at least 2 billion, at least 3 billion, at least 4 billion, at least 5 billion, at least 6 billion, at least 7 billion, at least 8 billion, at least 9 billion, at least 10 billion, at least 15 billion, at least 20 billion, at least 30 billion, at least 40 billion, at least 50 billion, at least 60 billion, at least 70 billion, at least 80 billion, at least 90 billion, or at least 100 billion hepatocytes, Genetically modified hepatocytes may be delivered to a subject in need thereof in a single dose or in multiple doses,

The specific dose level and frequency of dosage for any particular subject may be varied and will depend upon a variety of factors, including the activity of the cells of the composition(s), the stability and length of action of the cells of the composition, the age, body weight, general health, sex and diet of the subject, mode and time of administration, drug combination(s) co-administered, and severity of the condition of the host undergoing therapy,

The above listed examples of therapies should not be construed as limiting and essentially any appropriate therapy resulting in the desired therapeutic outcome in subjects identified as described may be employed,

The hepatocyte-like cells provided herein can be used for therapy of any subject in need of having hepatic function restored or supplemented, Human conditions that may be appropriate for such therapy include, but are not limited to, fulminant hepatic failure due to any cause, viral hepatitis, drug-induced liver injury, cirrhosis, inherited hepatic insufficiency (such as Wilson's disease, Gilbert's syndrome, or α1-antitrypsin deficiency), hepatobiliary carcinoma, autoimmune liver disease (such as autoimmune chronic hepatitis or primary biliary cirrhosis), and any other condition that results in impaired hepatic function,

In some embodiments, the hepatocyte-like cells provided herein are encapsulated or part of a bioartificial liver device, Bioartificial organs for clinical use are designed to support an individual with impaired liver function—either as a part of long-term therapy, or to bridge the time between a fulminant hepatic failure and hepatic reconstitution or liver transplant, Bioartificial liver devices are disclosed, for example, in U,S, Pat, Nos, 5,290,684, 5,624,840, 5,837,234, 5,853,717, and 5,935,849, Suspension-type bioartificial livers comprise cells suspended in plate dialysers, microencapsulated in a suitable substrate, or attached to microcarrier beads coated with extracellular matrix, Alternatively, hepatocytes can be placed on a solid support in a packed bed, in a multiplate flat bed, on a microchannel screen, or surrounding hollow fiber capillaries, The device has an inlet and outlet through which the subject's blood is passed, and sometimes a separate set of ports for supplying nutrients to the cells,

Hepatocytes are prepared according to the methods described herein, and then plated into the device on a suitable substrate, such as a matrix of Matrigel® or collagen, The efficacy of the device can be assessed by comparing the composition of blood in the afferent channel with that in the efferent channel—in terms of metabolites removed from the afferent flow, and newly synthesized proteins in the efferent flow,

Devices of this kind can be used to detoxify a fluid such as blood, wherein the fluid comes into contact with the hepatocytes provided in certain aspects of this technology under conditions that permit the cell to remove or modify a toxin in the fluid, The detoxification will involve removing or altering at least one ligand, metabolite, or other compound (either natural and synthetic) that is usually processed by the liver, Such compounds include but are not limited to bilirubin, bile acids, urea, heme, lipoprotein, carbohydrates, transferrin, hemopexin, asialoglycoproteins, hormones like insulin and glucagon, and a variety of small molecule drugs, The device can also be used to enrich the efferent fluid with synthesized proteins such as albumin, acute phase reactants, and unloaded carrier proteins, The device can be optimized so that a variety of these functions is performed, thereby restoring as many hepatic functions as are needed, In the context of therapeutic care, the device processes blood flowing from a patient in hepatocyte failure, and then the blood is returned to the patient,

### C, T Cells

T cells to be used in a cell therapy product may be profiled for donor capability at any stage of the manufacturing process of the cell therapy product,

T cells used in a cell therapy product may be primary T cells, Methods for profiling a population of cells for donor capability as described anywhere herein may be performed on primary (e,g,, genome-edited) T cells,

As described elsewhere herein, T cells used in a cell therapy product may be pluripotent stem cell (iPSC)-derived T cells, Methods for profiling a population of cells for donor capability as described anywhere herein may also be performed on stem cells capable of differentiating to form T cells, Methods for profiling a population of cells for donor capability as described anywhere herein may also be performed on stem cell derived (e,g,, genome-edited) T cells,

Relevant information concerning T cells as referred to in the context of the present disclosure is known in the art, including certain information regarding desired features of T cells when used for cell therapy, It will be understood that embodiments concerning T cells described herein may be readily and appropriately combined It will be understood that embodiments concerning T cells described herein may be readily and appropriately combined with embodiments describing HIP cells (e,g,, exhibiting reduced expression of one or more molecules of the MHC class I and/or MHC class II molecules and increased expression of at least one tolerogenic factor), as well as embodiments describing safety switches, and other modified/gene (e,g, CAR transgene) edited cells as described herein, T cells to be used in a cell therapy product may be profiled for donor capability at any stage of the manufacturing process of the cell therapy product, T cells to be used in a cell therapy product may be profiled for donor capability at any stage of the editing process during manufacturing of the cell therapy product,

The T cells described herein may be used to treat or prevent a disease in a subject,

In some embodiments, the cells that are engineered or modified as provided herein are primary T lymphocytes (also called T cells), In some embodiments, the primary T lymphocytes are isolated or obtained from one or more individual donor subjects, such as one or more individual healthy donor (e,g,, a subject that is not known or suspected of, e,g,, not exhibiting clinical signs of, a disease or infection), In some instances, the T cells are populations of primary T cells from one or more individuals, In some instances, the T cells are subpopulations or sub-types or subsets of primary T cells from one or more individuals, Subpopulations and sub-types and subsets of primary T cells are herein described in further detail below, As will be appreciated by those in the art, methods of isolating or obtaining T lymphocytes from an individual can be achieved using known techniques, Provided herein are engineered primary T lymphocytes that contain modifications (e,g,, genetic modifications) described herein for subsequent transplantation or engraftment into subjects (e,g,, recipients),

In some embodiments, primary T cells are obtained (e,g,, harvested, extracted, removed, or taken) from a subject or an individual, In some embodiments, primary T cells are produced from a pool of T cells such that the T cells are from one or more subjects (e,g,, one or more human including one or more healthy humans), In some embodiments, the pool of primary T cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects, In some embodiments, the donor subject is different from the patient (e,g,, the recipient that is administered the therapeutic cells), In some embodiments, the pool of T cells does not include cells from the patient, In some embodiments, one or more of the donor subjects from which the pool of T cells is obtained are different from the patient,

In some embodiments, the cells as provided herein are T lymphocytes differentiated from engineered pluripotent cells that contain modifications (e,g,, genetic modifications) described herein and that are differentiated into T lymphocyte, As will be appreciated by those in the art, the methods for differentiation depend on the desired cell type using known techniques, In some embodiments, the cells differentiated into a T lymphocyte may be used for subsequent transplantation or engraftment into subjects (e,g,, recipients),

Methods for generating T cells from pluripotent stem cells (e,g,, iPSC) are described, for example, in Iriguchi et al,, Nature Communications 12, 430 (2021); Themeli et al, 16(4):357-366 (2015); Themeli et al,, Nature Biotechnology 31:928-933 (2013),

Non-limiting examples of primary T cells include CD3+ T cells, CD4+ T cells, CD8+ T cells, naïve T cells, regulatory T (Treg) cells, non-regulatory T cells, Th1 cells, Th2 cells, Tb9 cells, Th17 cells, T-follicular helper (Tfh) cells, cytotoxic T lymphocytes (CTL), effector T (Teff) cells, central memory T (Tcm) cells, effector memory T (Tem) cells, effector memory T cells express CD45RA (TEMRA cells), tissue-resident memory (Trm) cells, virtual memory T cells, innate memory T cells, memory stem cell (Tsc), γδ T cells, and any other subtype of T cells, In some embodiments, the primary T cells are selected from a group that includes cytotoxic T-cells, helper T-cells, memory T-cells, regulatory T-cells, tumor infiltrating lymphocytes, and combinations thereof,

In some embodiments, primary T cells are primary T cells of a T cell subset or a sub-type or subpopulation, Such T cell subsets may be found in US2018/0319862A1 and/or WO2016/090190A1, the entire contents of which are hereby incorporated by reference, Accordingly, in some embodiments, the T cell subset, such as a CD62L+ T cell subset, that is increased in subjects upon administration of the genetically engineered cells are or include or share phenotypic characteristics with memory T cells or particular subsets thereof, such as long-lived memory T cells, In some embodiments, such memory T cells are central memory T cells (Tcm) or T memory stem cells (Tsc) cells, In some embodiments, the memory T cells are Tsc cells, Tsc cells may be described as having one or more phenotypic differences or functional features compared to other memory T cell subsets or compared to naïve T cells, such as being less differentiated or more naïve (see e,g,, Ahlers and Belyakov (2010) Blood, 115:1678), Cieri et al (2015) Blood, 125:2865: Flynn et al, (2014) Clinical & Translational Immunology, 3, e20; Gattinoni et al, (2012) Nat, Med,, 17:1290-1297; Gattinoni et al, (2012) Nat, Reviews, 12:671; Li et al, (2013) PLOS ONE, 8:e67401; and published PCT Appl, No, WO2014/039044), In some cases, Tsc cells are thought to be the only memory T cells able to generate effector T cells and all three subsets of memory T cells (Tsc, Tcm, and Tem), In some aspects, Tsc cells have the highest survival and proliferation response to antigenic or homeostatic stimuli of all the memory T cell subsets, and the least attrition absent cognate antigen, In some embodiments, the less-differentiated Tsc cells may exhibit greater expansion, long-term viability, and target cell destruction following adoptive transfer than other memory T cells, and thus may be able to mediate more effective treatment with fewer transferred cells than would be possible for either Tcm or Tem cells,

Among the sub-types and subpopulations of T cells and/or of CD4+ and/or of CD8+ T cells are naïve T (Tn) cells, effector T cells (Teff), memory T cells and sub-types thereof, such as stem cell memory T (Tsc), central memory T (Tcm), effector memory T (Tem), or terminally differentiated effector memory T cells, tumor-infiltrating lymphocytes (Til), immature T cells, mature T cells, helper T cells, cytotoxic T cells, mucosa-associated invariant T (MAIT) cells, naturally occurring and adaptive regulatory T (Treg) cells, helper T cells, such as TH 1 cells, TH2 cells, TH3 cells, TH 17 cells, TH9 cells, TH22 cells, follicular helper T cells, alpha/beta T cells, and delta/gamma T cells,

In some embodiments, CD8+ cells are further enriched for or depleted of naïve, central memory, effector memory, and/or central memory stem cells, such as by positive or negative selection based on surface antigens associated with the respective subpopulation, In some embodiments, enrichment for central memory T (Tcm) cells is carried out to increase efficacy, such as to improve long-term survival, expansion, and/or engraftment following administration, which in some aspects is robust in such sub-populations, See Terakura et al, (2012) Blood, 1: 72-82; Wang et al, (2012) J Immunother, 35 (9): 689-701, In some embodiments, combining Tcm-enriched CD8+ T cells and CD4+ T cells further enhances efficacy,

The present disclosure relates to adoptive cell therapy involving the administration of multiple doses of cells expressing genetically engineered (recombinant) receptors, e,g,, via multiple administration steps and/or by administration to subjects having received a prior administration, Methods of treatment and cell therapy are described herein in further detail below, In some embodiments, the noted differences are the only differences or substantially or essentially the only differences, between the recombinant molecule, e,g,, receptor, in the cells of the first dose or administration as compared to the second dose or administration, In some embodiments, aside from differences in the receptor and/or other noted differences, the cells and/or cell populations administered in a prior and subsequent administration are identical or essentially or substantially identical, In some embodiments, the ratio of cells expressing detectable surface levels of one or more markers is the same or similar in one administration as compared to the subsequent administration, In some embodiments, the percentages of populations and/or sub-populations of cells in the different doses or administrations are the same or substantially or essentially the same, The different doses may contain the same percentage of T cells, CD8+ and/or CD4+ T cells, T cells of a particular lineage or activation state or experience, such as relative percentages of effector, naïve, and/or memory T cells, and/or subpopulations thereof such as Tcm, Tem, Tsc cells and/or the cells may be derived from the same subject, sample, tissue, and/or fluid or compartment, In some embodiments, another portion of the same composition of cells used to engineer the cells of the first dose, e,g,, by transduction with a vector encoding the recombinant receptor, is used to engineer the cells of the second administration, In some embodiments, the composition is preserved, e,g,, by cryopreservation, prior to the second administration,

Among the sub-types and subpopulations of T cells and/or of CD4+ and/or of CD8+ T cells are naïve T (Tn) cells, effector T cells (Teff), memory T cells and sub-types thereof, such as stem cell memory T (Tsc), central memory T (Tcm), effector memory T (Tem), or terminally differentiated effector memory T cells, tumor-infiltrating lymphocytes (Til), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells,

Exemplary T cells of the present disclosure are selected from the group consisting of cytotoxic T cells, helper T cells, memory T cells, central memory T cells, effector memory T cells, effector memory RA T cells, regulatory T cells, tissue infiltrating lymphocytes, and combinations thereof, In many embodiments, the T cells express CCR7, CD27, CD28, and CD45RA, In some embodiments, the central T cells express CCR7, CD27, CD28, and CD45RO, In other embodiments, the effector memory T cells express PD-1, CD27, CD28, and CD45RO, In other embodiments, the effector memory RA T cells express PD-1, CD57, and CD45RA,

In some embodiments, the engineered T cells described herein, such as primary T cells isolated from one or more individual donors (e,g,, healthy donors) or T cells differentiated from iPSCs derived from one or more individual donors (e,g,, healthy donors), comprise T cells engineered (e,g,, are modified) to express a chimeric antigen receptor including but not limited to a chimeric antigen receptor described herein, Any suitable CAR can be included in the T cells, including the CARs described herein, In some embodiments, the engineered T cells express at least one chimeric antigen receptor that specifically binds to an antigen or epitope of interest expressed on the surface of at least one of a damaged cell, a dysplastic cell, an infected cell, an immunogenic cell, an inflamed cell, a malignant cell, a metaplastic cell, a mutant cell, and combinations thereof, In other cases, the engineered T cell comprise a modification causing the cell to express at least one protein that modulates a biological effect of interest in an adjacent cell, tissue, or organ when the cell is in proximity to the adjacent cell, tissue, or organ, Useful modifications to T cells, including primary T cells, are described in detail in US2016/0348073 and WO2020/018620, the disclosures of which are incorporated herein in their entireties,

In some embodiments, the T cell includes a polynucleotide encoding a CAR, wherein the polynucleotide is inserted in a genomic locus, Any suitable method can be used to insert the CAR into the genomic locus of the T cell including lentiviral based transduction methods or gene editing methods described herein (e,g,, a CRISPR/Cas system), In some embodiments, the polynucleotide is inserted into a safe harbor locus, such as but not limited to, an AAVS1, CCR5, CLYBL, ROSA26, SHS231, F3 (also known as CD142), MICA, MICB, LRP1 (also known as CD91), HMGB1, ABO, RHD, FUT1, or KDM5D gene locus, In some embodiments, the polynucleotide is inserted in a B2M, CIITA, TRAC, TRBC, PD1 or CTLA4 gene,

In some embodiments, the T cells described herein such as the engineered or modified T cells comprise reduced expression of an endogenous T cell receptor, In some embodiments, the TRAC or TRBC locus is disrupted or eliminated in the cell, such as by gene editing methods described herein (e,g,, a CRISPR/Cas system), In some embodiments, an exogenous polynucleotide or transgene, such as a polynucleotide encoding a CAR or other polynucleotide as described, is inserted into the disrupted TRAC or TRBC locus,

In some embodiments, the T cells described herein such as the engineered or modified T cells include reduced expression of cytotoxic T-lymphocyte-associated protein 4 (CTLA4), In some embodiments, the CTLA-4 locus is disrupted or eliminated in the cell, such as by gene editing methods described herein (e,g,, a CRISPR/Cas system), In some embodiments, an exogenous polynucleotide or transgene, such as a polynucleotide encoding a CAR or other exogenous polynucleotide as described, is inserted into the disrupted CTLA-4 locus,

In other embodiments, the T cells described herein such as the engineered or modified T cells include reduced expression of programmed cell death (PD1), In some embodiments, the PD1 locus is disrupted or eliminated in the cell, such as by gene editing methods described herein (e,g,, a CRISPR/Cas system), In some embodiments, an exogenous polynucleotide or transgene, such as a polynucleotide encoding a CAR or other exogenous polynucleotide as described, is inserted into the disrupted PD1 locus, In certain embodiments, the T cells described herein such as the engineered or modified T cells include reduced expression of CTLA4 and PD1,

In certain embodiments, the T cells described herein such as the engineered or modified T cells include enhanced expression of PD-L1, In some embodiments, the PD-L1 locus is disrupted or eliminated in the cell, such as by gene editing methods described herein (e,g,, a CRISPR/Cas system), In some embodiments, an exogenous polynucleotide or transgene, such as a polynucleotide encoding a CAR or other exogenous polynucleotide as described, is inserted into the disrupted PD-L 1 locus,

In some embodiments, the present technology is directed to engineered T cells, such as primary T cells isolated from one or more individual donors (e,g,, healthy donors) or T cells differentiated from iPSCs derived from one or more individual donors (e,g,, healthy donors), that overexpress a tolerogenic factor (e,g,, CD47), have reduced expression or lack expression of one or more MHC class I molecules and/or one or more MHC class II molecules (e,g,, one or more MHC class I human leukocyte antigens and/or one or more MHC class II human leukocyte antigens),In some embodiments, the engineered T cells further express one or more complement inhibitors, In some embodiments, the engineered T cells also are engineered to express a CAR, In some embodiments, the engineered T cells have reduced expression or lack expression of TCR complex molecules, such as by a genomic modification (e,g,, gene disruption) in the TRAC gene or TRBC gene, In some embodiments, T cells overexpress a tolerogenic factor (e,g,, CD47) and a CAR and harbor genomic modifications that disrupt one or more of the following genes: the B2M, CIITA, TRAC and TRBC genes,

In some embodiments, the provided engineered T cells evade immune recognition, In some embodiments, the engineered T cells described herein, such as primary T cells isolated from one or more individual donors (e,g,, healthy donors) or T cells differentiated from iPSCs derived from one or more individual donors (e,g,, healthy donors), do not activate an immune response in the patient (e,g,, recipient upon administration), Provided are methods of treating a disease by administering a population of engineered T cells described herein to a subject (e,g,, recipient) or patient in need thereof,

T cells provided herein are useful for the treatment of suitable cancers including, but not limited to, B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer,

### D, Natural Killer Cells

NK cells to be used in a cell therapy product may be profiled for donor capability at any stage of the manufacturing process of the cell therapy product,

NK cells used in a cell therapy product may be primary NK cells, Methods for profiling a population of cells for donor capability as described anywhere herein may be performed on primary (e,g,, genome-edited) NK cells,

As described elsewhere herein, NK cells used in a cell therapy product may be pluripotent stem cell (iPSC)-derived NK cells, Methods for profiling a population of cells for donor capability as described anywhere herein may also be performed on stem cells capable of differentiating to form NK cells, Methods for profiling a population of cells for donor capability as described anywhere herein may also be performed on stem cell derived (e,g,, genome-edited) NK cells,

Relevant information concerning NK cells as referred to in the context of the present disclosure is known in the art, including certain information regarding desired features of NK cells when used for cell therapy and for example may be found from WO2017214569A1, WO 2011/068896 A1, the contents of which are herein incorporated by reference, It will be understood that embodiments concerning NK cells described herein may be readily and appropriately combined with embodiments describing HIP cells (e,g,, exhibiting reduced expression of one or more molecules of the MHC class I and/or MHC class II molecules and increased expression of at least one tolerogenic factor), as well as embodiments describing safety switches, and other modified/gene (e,g, CAR transgene) edited cells as described herein, The NK cells described herein may be used to treat or prevent a disease in a subject, NK cells to be used in a cell therapy product may be profiled for donor capability at any stage of the editing process during manufacturing of the cell therapy product,

In some embodiments, the cells that are engineered or modified as provided herein are Natural Killer (NK) cells, In some embodiments, the NK cells are isolated or obtained from one or more individual donor subjects, such as one or more individual healthy donor (e,g,, a subject that is not known or suspected of, e,g,, not exhibiting clinical signs of, a disease or infection), In some instances, the NK cells are populations or subpopulations of NK cells from one or more individuals, As will be appreciated by those in the art, methods of isolating or obtaining NK cells from an individual can be achieved using known techniques, Provided herein are engineered primary NK cells that contain modifications (e,g,, genetic modifications) described herein for subsequent transplantation or engraftment into subjects (e,g,, recipients, For instance, the engineered T cells are administered to a subject (e,g,, recipient, such as a patient), by infusion of the engineered NK cells into the subject,

In some embodiments, the cells as provided herein are NK cells differentiated from engineered pluripotent cells that contain modifications (e,g,, genetic modifications) described herein and that are differentiated into NK cells, As will be appreciated by those in the art, the methods for differentiation depend on the desired cell type using known techniques, In some embodiments, the cells differentiated into an NK cells may be used for subsequent administration to a subject (e,g,, recipient, such as a patient), such as by infusion of the differentiated NK cells into the subject,

Methods for generating NK cells from pluripotent stem cells (e,g,, iPSC) are described, for example, in U,S, patent Ser, No, 10/626,373; Shankar et al, Stem Cell Res Ther, 2020; 11: 234: Euchner et al, Frontiers in Immunology, 2021; 12, Article 640672, doi=10,3389/fimmu,2021,640672;

In some embodiments, NK cells are obtained (e,g,, harvested, extracted, removed, or taken) from a subject or an individual, In some embodiments, NK cells are produced from a pool of NK cells such that the NK cells are from one or more subjects (e,g,, one or more human including one or more healthy humans), In some embodiments, the pool of primary NK cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects, In some embodiments, the donor subject is different from the patient (e,g,, the recipient that is administered the engineered NK cells), In some embodiments, the pool of NK cells does not include cells from the patient, In some embodiments, one or more of the donor subjects from which the pool of NK cells is obtained are different from the patient,

In some embodiments, NK cells, including primary NK cells isolated from one or more individual donors (e,g,, healthy donors) or NK cells differentiated from iPSCs derived from one or more individual donors (e,g,, healthy donors) express CD56 (e,g,, CD56^{dim} or CD56^{bright}) and lack CD3 (e,g,, CD3^{neg}, In some embodiments, NK cells as described herein may also express the low-affinity Fcγ receptor CD16, which mediate ADCC, In some embodiments, the NK cells also express one or more natural killer cell receptors NKG2A and NKG2D or one or more natural cytotoxicity receptors NKp46, NKp44, NKp30, For example, for the case of primary NK cells, in specific cases, the primary cells may be isolated from a starting source of NK cells, such as a sample containing peripheral blood mononuclear cells (PBMCs), by depletion of cells positive for CD3, CD14, and/or CD19, For instance, the cells may be subject to depletion using immunomagnetic beads having attached thereto antibodies to CD3, CD14, and/or CD19, respectively), thereby producing an enriched population of NK cells, In other cases, primary NK cells may be isolated from a starting source that is a mixed population (e,g,, PBMCs) by selecting cells for the presence of one or more markers on the NK cells, such as CD56, CD16, NKp46, and/or NKG2D,

In some embodiments, prior to the engineering as described herein, the NK cells, such as isolated primary NK cells, may be subject to one or more expansion or activation step, In some embodiments, expansion may be achieved by culturing of the NK cells with feeder cells, such as antigen presenting cells that may or may not be irradiated, The ratio of NK cells to antigen presenting cells (APCs) in the expansion step may be of a certain number, such as 1:1, 1:1,5, 1:2, or 1:3, for example, In certain aspects, the APCs are engineered to express membrane-bound IL-21 (mblL- 21), In aspects, the APCs are alternatively or additionally engineered to express IL-21, IL-15, and/or IL-2, In embodiments, the media in which the expansion step(s) occurs comprises one or more agents to facilitate expansion, such as one or more recombinant cytokines, In specific embodiments, the media comprises one or more recombinant cytokines from IL-2, IL-15, IL-18, and/or IL-21, In some embodiments, the steps for engineered the NK cells by introducing the modifications as described herein is carried out 2-12 days after initiation of the expansion, such as on or about day 2, 3, 4, 5, 6, 7, 8, 9, 10, 11 or 12,

In some embodiments, the engineered NK cells described herein, such as primary NK cells isolated from one or more individual donors (e,g,, healthy donors), comprise NK cells engineered (e,g,, are modified) to express a chimeric antigen receptor including but not limited to a chimeric antigen receptor described herein, Any suitable CAR can be included in the NK cells, including the CARs described herein, In some embodiments, the engineered NK cells express at least one chimeric antigen receptor that specifically binds to an antigen or epitope of interest expressed on the surface of at least one of a damaged cell, a dysplastic cell, an infected cell, an immunogenic cell, an inflamed cell, a malignant cell, a metaplastic cell, a mutant cell, and combinations thereof, In other cases, the engineered NK cell comprise a modification causing the cell to express at least one protein that modulates a biological effect of interest in an adjacent cell, tissue, or organ when the cell is in proximity to the adjacent cell, tissue, or organ,

In some embodiments, the NK cell includes a polynucleotide encoding a CAR, wherein the polynucleotide is inserted in a genomic locus, Any suitable method can be used to insert the CAR into the genomic locus of the NK cell including lentiviral based transduction methods or gene editing methods described herein (e,g,, a CRISPR/Cas system), In some embodiments, the polynucleotide is inserted into a safe harbor locus, such as but not limited to, an AAVS1, CCR5, CLYBL, ROSA26, SHS231, F3 (also known as CD142), MICA, MICB, LRP1 (also known as CD91), HMGB1, ABO, RHD, FUT1, or KDM5D gene locus,

In some embodiments, the present technology is directed to engineered NK cells, such as primary NK cells isolated from one or more individual donors (e,g,, healthy donors) or NK cells differentiated from iPSCs derived from one or more individual donors (e,g,, healthy donors), that overexpress a tolerogenic factor (e,g,, CD47), have reduced expression or lack expression of one or more MHC class I molecules and/or one or more MHC class II molecules (e,g,, one or more MHC class I human leukocyte antigens and/or one or more MHC class II human leukocyte antigens),

In some embodiments, the provided engineered NK cells evade immune recognition, In some embodiments, the engineered NK cells described herein, such as primary NK cells isolated from one or more individual donors (e,g,, healthy donors), do not activate an immune response in the patient (e,g,, recipient upon administration), Provided are methods of treating a disease by administering a population of engineered NK cells described herein to a subject (e,g,, recipient) or patient in need thereof,

NK cells provided herein are useful for the treatment of suitable cancers including, but not limited to, B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer,

Cells disclosed herein may be natural killer (NK) cells, for example a population of NK cells, It will be understood that any reference to “a cell” e,g, “a NK cell” below also applies to “a population of cells” e,g, “a population of NK cells” as described in the present application,

NK cells, which arise through the lymphoid lineage and are part of the innate immune system, may be used in anti-cancer therapy as they have been found to detect and kill certain types of tumor cells,

Natural killer (NK) cells are cytotoxic lymphocytes capable of human immune surveillance, WO2017214569A1 (the contents of which are incorporated herein by reference in their entirety) describes genome-edited primary NK cells, methods of making those cells, and methods of administering those cells,

In some embodiments, the NK cells can be immature NK cells and can be CD56+ and CD16−, In some embodiments, the NK cells can be mature NK cells and can be CD56− and CD16+, or CD561o and CD16+ (as described in WO 2011/068896A1 (the contents of which are incorporated herein by reference in their entirety)),

A primary NK cell may express CD16 and/or CD56, In some embodiments, an NK cell does not express CD3, In some embodiments, an “NK cell” is preferably defined as a cell that is CD56+ and CD3−, In some embodiments, an “NK cell” is defined as a cell that is CD16+ and CD3−, NK cells are lymphocytes of the innate immune system that kill virally infected or transformed cells, Like T cells, NK cells are cytotoxic lymphocytes, Unlike T cells, NK cells do not require antigen recognition, and require integration of signals from many activating and inhibitory receptors to perform their function, Despite their similarities to T cells, NK cells behave differently under stimulation conditions and do not tolerate electroporation in the same way as T cells (as described in WO2017214569A1 (the contents of which are incorporated herein by reference in their entirety)),

Methods of generating natural killer (NK) cells are known in the art, for example methods described in WO 2011/068896A1 (the contents of which are incorporated by reference herein in their entirety) comprising: providing hemangioblasts; culturing the hemangioblasts on methylcellulose and a first cytokine mixture comprising IL2, IL3, IL6, IL7, IL15, SCF and FL; harvesting the cultured cells; and culturing the harvested cells in liquid media comprising human serum, and a second cytokine mixture comprising IL7, IL15, SCF and FL to generate NK cells,

A primary NK cell may be isolated from, for example, peripheral blood, umbilical cord cells, ascites, and/or a solid tumor (as described in WO2017214569A1, the contents of which are incorporated herein by reference in their entirety), In some embodiments, a “primary NK cell” is an NK cell that is freshly isolated, In some embodiments, a “primary NK cell” is an NK cell that has undergone up to 5 replications or divisions after being isolated, up to 10 replications or divisions after being isolated, up to 15 replications or divisions after being isolated, up to 20 replications or divisions after being isolated, up to 25 replications or divisions after being isolated, up to 30 replications or divisions after being isolated, up to 35 replications or divisions after being isolated, or up to 40 replications or divisions after being isolated, In some embodiments, the primary NK cell is a non-clonal cell, In some embodiments, primary NK cell is a proliferating cell, In some embodiments, primary NK cell is an expanded cell,

In some embodiments, the NK cell is a mammalian cell, In some embodiments, the NK cell is preferably a human cell, In some embodiments, the NK cell is a mouse cell,

Some desired features of NK cells when used for cell therapy are described herein,

NK cells may be distinguished using a number of assays, for example as set out in WO 2011/068896A1 (the contents of which is hereby incorporated by reference in its entirety),

For example, IL12-p70 secretion may be measured: IL12p70 is secreted by mature DCs in order to elicit a Th1-directed response from CD4+ T cells. Human BM-derived DCs can produce >500pg/ml of IL12p70 yet blast-derived DCs did not produce any detectable IL12p70 upon maturation. Mixed lymphocyte reaction (MLR) assay: The MLR assay determines the ability of DCs to stimulate proliferation of allogenic T cells. Cord blood mononuclear cells (CBMCs, which include T cells) were used as responders, fluorescently labeled, and their proliferation was measured after 4-5 days coculture with immature or mature blast-derived DCs. Preliminary results show that the responder cells proliferate in response to mature (m)DCs. Hemangioblast-derived natural killer cells: Cell surface markers CD45, CD7, CD94, CD56, CD16, and NKG2D were evaluated in blast-derived and human bone marrow-derived NK cells.

Methods of differentiating NK cells are known in the art, for example as described in WO 2011/068896A1 (the contents of which are hereby incorporated by reference in their entirety). An exemplary method as described in WO 2011/068896A1 is set out below:

Initial differentiation as described in WO 2011/068896A1: The initial differentiation procedure for both cell types may be the same and may involve a 4 day culture of hESCs in Stemline II (Sigma) plus cytokines in order to generate embryoid bodies (EBs). The cytokines. VEGF and BMP4 may be used throughout the EB culture while bFGF may be added after the first 2 days. After 4 days total, the resulting EBs may be disaggregated with 0.05% trypsin and then the trypsin may be inactivated with serum- containing media. Individual cells may be subsequently filtered through a 40 μM cell strainer, counted, and seeded into H4436 or H4536 methylcellulose (Stem Cell Technologies) containing additional cytokines, such as TPO (50pg/ml), VEGF(50pg/ml), FL (50pg/ml) and bFGF (20-50pg/ml). For NK differentiation, the cytokines IL2 (1-10 μg/ml), IL7 (1-20 μg/ml), and/or IL 15 (1-10 μg/ml) may also be added at this stage. Methylcellulose cultures may be plated at a concentration of 50,000 to 150,000 cells per ml for the production and expansion of a hemangioblastic population. Blast-like cells may be harvested from methylcellulose between day 6 and 10 and further differentiated by one of the following procedures.

NK differentiation as described in WO 2011/068896A1: Blast cells may be replated in H4236 methylcellulose plus IL2 (5-10 ng/ml), IL3 (1-10 ng/ml), IL6 (1-10 ng/ml), IL7 (5-20 ng/ml), IL15 (5-10 ng/ml), SCF (10-50 ng/ml), and FL (10-50 ng/ml) or in liquid culture containing the same cytokines and 10-20% human serum. After 6-8 days culture, cells may be harvested and replated in liquid media (aMEM or DMEM:F12) plus 10-20% human serum and the cytokines IL7 (5-20 ng/ml), IL15 (5-10 ng/ml). SCF (10-50 ng/ml), and FL(10-50 ng/ml) for an additional 14-21 days. Weekly media changes may be used to refresh the cytokine cocktail.

Flow cytometry may be used intermittently throughout the differentiation procedure to assess the immunophenotype of cells and the acquisition of NK cell surface markers. Cell surface markers include CD34, CD45. CD56, CD16, CD94. NKG2D, CD3. CD7, CD4, CD8a, and CD45RA. Tests to examine the function of hemangioblast-derived NK cells may include (1) natural cytotoxicity assay using K562 erythro leukemia target cells, (2) IFNγ production in response to IL12/IL18 or phorbol myristate acetate treatment, (3) intracellular flow cytometry for presence of perforin and granzyme B enzymes, and (4) antibody-dependent cellular cytotoxicty assay using Raji cells and anti-CD20 antibodies. NK cells may be generated from both H7 and HuES3 hESCs. A non-radioactive cytotoxicity assay similar to the 51Cr release assay may show that hemangioblast-derived NK cells harbor natural cytotoxicity function as they may be able to effectively induce apoptosis in target K562 erythroblastic leukemia cells after a standard 4 hr co-culture.

Alteration of hemangioblast growth conditions as described in WO 2011/068896A1: The above NK and DC differentiation procedures may be performed using hemangioblasts grown in H4436 methylcellulose. However, H4536, an erythropoietin- free methylcellulose from Stem Cell Technologies can also be used to efficiently generate hemangioblasts. These “epo minus” hemangioblasts are quite similar to the original “epo plus” blasts; they are capable of differentiating into a variety of hematopoietic and vascular cell types. Preliminary results as described in WO 2011/068896A1 suggest that the use of H4536 may provide a significant advantage over H4436 methylcellulose for the differentiation of hemangioblasts into various hematopoietic lineages, including NK cells and DCs. The absence of epo in the blast growth media may be found to reduce the percentage of cells expressing the erythrocyte marker CD235a and increase the percentage of cells expressing CD34, CD45, and CD41a. Due to this difference in cell surface marker expression. “epo-minus” growth conditions may enhance differentiation down myeloid and/or lymphoid lineages.

NK cell differentiation from human ESCs as described in WO 2011/068896A1:

Differentiation procedure may be performed as follows: H7 ESCs were differentiated into embryoid bodies (EBs) for 4 days. EBs may be harvested and transferred to cytokine-rich methylcellulose for 10-15 days for hemangioblast production and expansion. Hemangioblasts may be harvested and placed into feeder- free liquid culture medium plus 10-20% human AB serum, with a panel of cytokines for an additional 14-17 days with media half changes every 3-4 days.

Immunopheno typing (using flow cytometry) as described in WO 2011/068896A1: Immature NK cells may be CD56bright, CD161o, KIRlo, CD117+, CD94−, NKG2D+. By using a variation of the above procedure, 20-30% of the viable cells after 32 days of differentiation may be CD56+CD16−. Mature NK cells may be CD56dim, CD16 hi, KIRhi, CD1171o/−, CD94+, NKG2D+. By using the above procedure, 20% of the viable cells after 31 days of differentiation may be CD56-CD16+ and 5% of them were CD561oCD16+.

Functional assays as described in WO 2011/068896A1: Natural cytotoxicity: mature NK cells can elicit apoptosis of target cells such as human K562 erythro leukemia, MCF7, U87, PC3, NTERA2 cells. A “3FC” assay may be used to assess efficiency of cytotoxicity. It is similar to 51Cr release assay but does not require radioactivity. See Derby et al., Immunol. Letters 78: 35-39 (2001). The heterogeneous population of mature NK cells described above (item B2-a) may be found to elicit apoptosis in 65-70% of K562 cells in a standard 4 hour experiment.

Antibody-dependent cell- mediated cytotoxicity (ADCC) as described in WO 2011/068896A1: The FcγRIII (CD16) on the NK cell surface may bind to the fc region of anti-CD20 antibodies attached to target cells and induces ADCC. Raji cells (derived from Burkett's lymphoma) may be preincubated with anti-CD20 antibody and used as targets in ADCC assay. (Tsirigotis et al., J of Steroid Biochem and Mol Bio 108: 267-271 (2008)).

IFNy cytokine production as described in WO 2011/068896A1: Immature NK cells may produce large amounts of IFNy in response to overnight treatment with PMA (phorbol myristate acetate) plus ionomycin or IL 12 plus IL 18. IFNy secretion may be blocked with brefeldin a, cells are stained for cell surface markers and IFNy using intracellular flow cytometry. See WoU et al. J. of Immunol. 175: 5095-5103 (2005).

In vivo immunotherapy potential of NK cells using xenograft mouse model as described in WO 2011/068896A1: Bio luminescent (luciferase-containing) K562 cells may be injected into NOD/SCID mice for engraftment of tumors, followed by bolus of NK cells and daily IP injections of IL2 and IL15. Bio luminescence imaging may be used to monitor in vivo NK immunotherapeutic potential over time. See: WoU et al. Blood 113 (24): 6094-6101 (2009)

%21 As described in WO 2011/068896A1, both cord blood and peripheral blood mononuclear cells may be used as responders and the inventors use human bone marrow-derived DCs as positive control effectors.

E4BP4 may be critical for NK lineage development (see Gascoyne et al. Nature Immunology 10(10): 1118-1125. 2009) and may provide the transcriptional program necessary for more efficient in vitro.

NK cell differentiation as described in WO 2011/068896A1: E4BP4 cDNA may be cloned into a retroviral vector for its overexpression in hemangioblasts and evaluate its ability to increase NK differentiation. RT-PCR may be used to monitor the expression of various KIR receptor isoforms and the enzymes, perforin and granzyme B, which are critical for NK cell functionality. For functional assays, blast-derived NK cells may display natural cytotoxicity, so their antibody-dependent cellular cytotoxicity (ADCC) capabilities may be assessed. Required reagents for the ADCC assay may include the Burkitt's lymphoma-derived Raji cells and anti-CD20 antibodies. Co-culture of CD20-marked Raji cells with NK cells should elicit a specific ADCC response, which may be be monitored through flow cytometric means.

An NK cell may be stimulated using any suitable method and for any suitable length of time (for example as described in WO2017214569A1 the contents of which are incorporated herein by reference in their entirety). In some embodiments, a stimulated NK cell includes an NK cell exposed to phorbol-12-myristate-13-acetate (PMA). In some embodiments, a stimulated NK cell includes an NK cell exposed to cytokines including, for example, IL-21, IL-2, IL-12, IL-15, type I interferons, etc. In some embodiments, the cytokine may include a soluble cytokine. In some embodiments, the cytokines are bound cytokines. In some embodiments, the cytokines may be bound to a surface (including, for example, the surface of a tissue culture flask).

In some embodiments, a bound cytokine may be bound to an artificial antigen presenting cell (aAPC). An aAPC can include, for example, clone 9, described by Denman et al., PLoS One, 2012, 7(1): e30264 doi: 10.1371/journal.pone.0030264. In some embodiments, an aAPC may be a bead. A spherical polystyrene bead may be coated with antibodies against NK cell surface proteins and be used for NK cell activation. A bead may be of any size. In some cases, the bead may be or may be 3 and 6 micrometers. A bead may be 4.5 micrometers in size. A bead may be utilized at any cell to bead ratio. For example, a 3 to 1 bead to cell ratio at 1 million cells per milliliter may be used. In some embodiments, an aAPC may be a rigid spherical particle, a polystyrene latex microbeads, a magnetic nano- or micro-particle, a nanosized quantum dot, a poly(lactic-co-glycolic acid) (PLGA) microsphere, a nonspherical particle, a carbon nanotube bundle, an ellipsoid PLGA microparticle, a nanoworm, a fluidic lipid bilayer-containing system, a 2D-supported lipid bilayer (2D-SLB5), a liposome, a RAFTsomes/microdomain liposome, an supported lipid bilayer particle, or any combination thereof.

In some embodiments, a stimulated NK cell includes an NK cell treated with a commercially available kit including, for example, CellXVivo Human NK Cell Expansion Kit (R&D Systems, Minneapolis, MN), Human NK Cell Expansion Activator Kit (Miltenyi Biotech, Bergisch Gladbach. Germany), etc.

In some embodiments, a stimulated NK cell includes an NK cell in a population that has been expanded at least 3 fold, at least 4 fold, at least 5 fold, at least 6 fold, at least 7 fold, or at least 8 fold. In some embodiments, a stimulated NK cell includes an NK cell in a population that has been expanded up to 5 fold, up to 6 fold, up to 7 fold, up to 8 fold, up to 10 fold, up to 20 fold, or up to 30 fold.

In some embodiments, the NK cells may be stimulated hours. In some embodiments, the NK cell may be stimulated for days. For example, in some embodiments, an NK cell may be co-cultured with an aAPC for up to 1 day, up to 2 days, up to 3 days, up to 4 days, up to 5 days, up to 6 days, up to 7 days, up to 8 days, or up to 9 days, up to 2 weeks, up to 3 weeks, and so forth.

In some embodiments, the method includes expanding an edited NK cell. In some embodiments, the expansion may be performed after selecting the NK cell. In some embodiments, an NK cell may be expanded by co-incubation with an artificial antigen-presenting cells (aAPC). In some embodiments, an NK cell may be expanded by co-incubation with an aAPC bound to a cytokine. In some embodiments, an NK cell may be expanded by co-incubation with a soluble cytokine. The cytokine may include, for example, IL-21, IL-2, IL-12, IL-15, type I interferons, etc. In some embodiments, an NK cell may preferably be expanded by co-incubation with an aAPC bound to IL-21 or expressing membrane-bound IL-21.

By using hemangioblasts as bone-marrow-repopulating cells or by differentiating them into dendritic, natural killer, T cells, and/or mesenchymal stem cells (MSCs), we can produce large-scale, effective cell-based therapies to combat cancer, HIV, and/or automimmune diseases.

Genome-edited primary NK cells or stem cell derived NK cells may be used to treat or prevent a disease in a subject (for example, as described in WO2017214569A1 the contents of which are incorporated herein by reference in their entirety). A method may include administering to the subject a composition that includes the genome-edited primary NK cell or stem cell derived NK cells described herein or produced by the method described herein. The disease could include, for example, cancer, a precancerous condition, infection with a pathogen (including, for example, malaria), or a viral infection. In some embodiments, it is preferred that the cells are used for cancer immunotherapy.

A genome-edited primary NK cell or stem cell derived NK cells may be administered to a subject alone or in combination with one or more other therapies. For example, a genome-edited primary NK cell or stem cell derived NK cells may be administered to a subject in combination a pharmaceutical composition that includes the active agent and a pharmaceutically acceptable carrier and/or in combination with a cellular therapy including, for example, a chimeric antigen receptor T cell (CAR-T). The NK cell may be administered to a patient, preferably a mammal, and more preferably a human, in an amount effective to produce the desired effect. The NK cell may be administered in a variety of routes, including, for example, intravenously, intratumorally, intraarterially, transdermally, via local delivery by catheter or stent, via a needle or other device for intratumoral injection, subcutaneously, etc. The NK cell may be administered once or multiple times. A physician having ordinary skill in the art may determine and prescribe the effective amount and dosing of an adaptive NK cell and, optionally, the pharmaceutical composition required.

The cancer may include, for example, bone cancer, brain cancer, breast cancer, cervical cancer, cancer of the larynx, lung cancer, pancreatic cancer, prostate cancer, skin cancer, cancer of the spine, stomach cancer, uterine cancer, hematopoietic cancer, and/or lymphoid cancer, etc. A hematopoietic cancer and/or lymphoid cancer may include, for example, acute myelogenous leukemia (AML), acute lymphoblastic leukemia (ALL), myelodysplastic syndromes (MDS), non- Hodgkin lymphoma (NHL), chronic myelogenous leukemia (CML), Hodgkin's disease, and/or multiple myeloma. The cancer may be a metastatic cancer.

The virus may include, for example, a herpes virus, including for example, CMV, Varicella zoster virus (VZV). Epstein-Barr virus (EBV), a herpes simplex virus (HSV) or Kaposi's sarcoma-associated herpesvirus (KSHV); or a lentivirus, including for example, human immunodeficiency virus (HIV). In a further aspect, a genome-edited primary NK cell may be administered to inhibit the growth of a tumor in a subject. In some embodiments, the tumor may include a solid tumor.

A genome-edited primary NK cell or stem cell derived NK cells may be administered or prepared in a subject before, during, and/or after other treatments. Such combination therapy may involve administering a genome-edited primary NK cell or stem cell derived NK cells before, during and/or after the use of other anti-cancer and/or antiviral agents including, for example, a cytokine; a chemokine: a therapeutic antibody including, for example, a high affinity anti-CMV IgG antibody; an NK cell receptor ligand, including, for example. BiKE or TRiKE; an adjuvant; an antioxidant; a chemotherapeutic agent; and/or radiation. The administration or preparation may be separated in time from the administration of other anti-cancer agents and/or anti-viral agents by hours, days, or even weeks. Additionally, or alternatively, the administration or preparation may be combined with other biologically active agents or modalities such as, but not limited to, an antineoplastic agent, and non-drug therapies, such as, but not limited to, surgery.

### E. Endothelial Cells

Endothelial cells to be used in a cell therapy product may be profiled for donor capability at any stage of the manufacturing process of the cell therapy product.

Endothelial cells used in a cell therapy product may be primary endothelial cells. Methods for profiling a population of cells for donor capability as described anywhere herein may be performed on primary (e.g., genome-edited) endothelial cells.

As described elsewhere herein, endothelial cells used in a cell therapy product may be pluripotent stem cell (iPSC)-derived beta-islet cells. Methods for profiling a population of cells for donor capability as described anywhere herein may also be performed on stem cells capable of differentiating to form endothelial cells. Methods for profiling a population of cells for donor capability as described anywhere herein may also be performed on stem cell derived (e.g., genome-edited) endothelial cells.

Relevant information concerning endothelial cells as referred to in the context of the present disclosure is known in the art, including certain information regarding desired features of endothelial cells when used for cell therapy. It will be understood that embodiments concerning endothelial cells described herein may be readily and appropriately combined with embodiments describing HIP cells (e.g., exhibiting reduced expression of one or more molecules of the MHC class I and/or MHC class II molecules and increased expression of at least one tolerogenic factor), as well as embodiments describing safety switches, and other modified/gene edited cells as described herein. Endothelial cells to be used in a cell therapy product may be profiled for donor capability at any stage of the editing process during manufacturing of the cell therapy product.

The endothelial cells described herein may be used to treat or prevent a disease in a subject.

In some embodiments, the cells that are engineered or modified as provided herein are primary endothelial cells. In some embodiments, the primary endothelial cells are isolated or obtained from one or more individual donor subjects, such as one or more individual healthy donor (e.g., a subject that is not known or suspected of, e.g., not exhibiting clinical signs of, a disease or infection). As will be appreciated by those in the art, methods of isolating or obtaining endothelial cells from an individual can be achieved using known techniques. Provided herein are engineered primary endothelial cell types that contain modifications (e.g., genetic modifications) described herein for subsequent transplantation or engraftment into subjects (e.g., recipients).

In some embodiments, primary endothelial cells are obtained (e.g., harvested, extracted, removed, or taken) from a subject or an individual. In some embodiments, primary endothelial cells are produced from a pool of endothelial cells such that the endothelial cells are from one or more subjects (e.g., one or more human including one or more healthy humans). In some embodiments, the pool of primary endothelial cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects. In some embodiments, the donor subject is different from the patient (e.g., the recipient that is administered the therapeutic cells). In some embodiments, the pool of endothelial cells does not include cells from the patient. In some embodiments, one or more of the donor subjects from which the pool of endothelial cells is obtained are different from the patient.

In some embodiments, the cells as provided herein are endothelial cells differentiated from engineered iPSCs that contain modifications (e.g., genetic modifications) described herein and that are differentiated into an endothelial cell type. As will be appreciated by those in the art, the methods for differentiation depend on the desired cell type using known techniques. In some embodiments, the cells differentiated into various endothelial cell types may be used for subsequent transplantation or engraftment into subjects (e.g., recipients).

In some embodiments, the engineered pluripotent cells described herein are differentiated into endothelial colony forming cells (ECFCs) to form new blood vessels to address peripheral arterial disease. Techniques to differentiate endothelial cells are known. See, e.g., Prasain et al., doi: 10.1038/nbt.3048, incorporated herein by reference in its entirety and specifically for the methods and reagents for the generation of endothelial cells from human pluripotent stem cells, and also for transplantation techniques. Differentiation can be assayed as is known in the art, generally by evaluating the presence of endothelial cell associated or specific markers or by measuring functionally.

In some embodiments, the method of producing a population of engineered endothelial cells from a population of engineered pluripotent cells by in vitro differentiation comprises: (a) culturing a population of engineered iPSCs cells in a first culture medium comprising a GSK inhibitor; (b) culturing the population of engineered iPSCs cells in a second culture medium comprising VEGF and bFGF to produce a population of pre-endothelial cells; and (c) culturing the population of pre-endothelial cells in a third culture medium comprising a ROCK inhibitor and an ALK inhibitor to produce a population of differentiated endothelial cells that are engineered to contain the modifications described herein.

In some embodiments, the GSK inhibitor is CHIR-99021, a derivative thereof, or a variant thereof. In some instances, the GSK inhibitor is at a concentration ranging from about 1 mM to about 10 mM. In some embodiments, the ROCK inhibitor is Y-27632, a derivative thereof, or a variant thereof. In some instances, the ROCK inhibitor is at a concentration ranging from about 1 pM to about 20 pM. In some embodiments, the ALK inhibitor is SB-431542, a derivative thereof, or a variant thereof. In some instances, the ALK inhibitor is at a concentration ranging from about 0.5 pM to about 10 pM.

In some embodiments, the first culture medium comprises from 2 pM to about 10 pM of CHIR-99021. In some embodiments, the second culture medium comprises 50 ng/ml VEGF and 10 ng/ml bFGF. In other embodiments, the second culture medium further comprises Y-27632 and SB-431542. In various embodiments, the third culture medium comprises 10 pM Y-27632 and 1 pM SB-431542. In certain embodiments, the third culture medium further comprises VEGF and bFGF. In instances, the first culture medium and/or the second medium is absent of insulin.

The cells provided herein can be cultured on a surface, such as a synthetic surface to support and/or promote differentiation of pluripotent cells into endothelial cells. In some embodiments, the surface comprises a polymer material including, but not limited to, a homopolymer or copolymer of selected one or more acrylate monomers. Non-limiting examples of acrylate monomers and methacrylate monomers include tetra(ethylene glycol) diacrylate, glycerol dimethacrylate, 1,4-butanediol dimethacrylate, poly(ethylene glycol) diacrylate, di(ethylene glycol) dimethacrylate, tetra(ethyiene glycol) dimethacrylate, 1,6-hexanediol propoxylate diacrylate, neopentyl glycol diacrylate, trimethylolpropane benzoate diacrylate, trimethylolpropane eihoxylate (1 EO/QH) methyl, tricyclo[5.2.1.0^{2,6}] decane dimethanol diacrylate, neopentyl glycol exhoxylate diacrylate, and trimethylolpropane triacrylate. Acrylate synthesized as known in the art or obtained from a commercial vendor, such as Polysciences, Inc., Sigma Aldrich. Inc. and Sartomer, Inc.

In some embodiments, the endothelial cells may be seeded onto a polymer matrix. In some cases, the polymer matrix is biodegradable. Suitable biodegradable matrices are well known in the art and include collagen-GAG, collagen, fibrin. PLA, PGA, and PLA/PGA co-polymers. Additional biodegradable materials include poly(anhydrides), poly(hydroxy acids), poly(ortho esters), poly(propylfumerates), poly(caprolactones), polyamides, polyamino acids, polyacetals, biodegradable polycyanoacrylates, biodegradable polyurethanes and polysaccharides.

Non-biodegradable polymers may also be used as well. Other non-biodegradable, yet biocompatible polymers include polypyrrole, polyanibnes, polythiophene, polystyrene, polyesters, non-biodegradable polyurethanes, polyureas, poly(ethylene vinyl acetate), polypropylene, polymethacrylate, polyethylene, polycarbonates, and poly(ethylene oxide). The polymer matrix may be formed in any shape, for example, as particles, a sponge, a tube, a sphere, a strand, a coiled strand, a capillary network, a film, a fiber, a mesh, or a sheet. The polymer matrix can be modified to include natural or synthetic extracellular matrix materials and factors.

The polymeric material can be dispersed on the surface of a support material. Useful support materials suitable for culturing cells include a ceramic substance, a glass, a plastic, a polymer or co-polymer, any combinations thereof, or a coating of one material on another. In some instances, a glass includes soda-lime glass, pyrex glass, vycor glass, quartz glass, silicon, or derivatives of these or the like.

In some instances, plastics or polymers including dendritic polymers include poly(vinyl chloride), poly(vinyl alcohol), poly(methyl methacrylate), poly(vinyl acetate-maleic anhydride), poly(dimethylsiloxane) monomethacrylate, cyclic olefin polymers, fluorocarbon polymers, polystyrenes, polypropylene, polyethyleneimine or derivatives of these or the like. In some instances, copolymers include poly(vinyl acetate-co-maleic anhydride), poly(styrene-co-maleic anhydride), poly(ethylene-co-acrylic acid) or derivatives of these or the like.

Additional descriptions of endothelial cells and their differentiation for use in the methods provided herein are found in WO2020/018615, the disclosure of which is herein incorporated by reference in its entirety.

In some embodiments, the population of engineered endothelial cells, such as primary endothelial cells isolated from one or more individual donors (e.g., healthy donors) or endothelial cells differentiated from iPSCs derived from one or more individual donors (e.g., healthy donors), are maintained in culture, in some cases expanded, prior to administration. In certain embodiments, the population of endothelial cells are cryopreserved prior to administration.

In some embodiments, the present technology is directed to engineered endothelial cells, such as primary endothelial cells isolated from one or more individual donors (e.g., healthy donors) or endothelial cells differentiated from iPSCs derived from one or more individual donors (e.g., healthy donors), that overexpress a tolerogenic factor (e.g., CD47), have reduced expression or lack expression of one or more MHC class I molecules and/or one or more MHC class II molecules (e.g., one or more MHC class I human leukocyte antigens and/or one or more MHC class II human leukocyte antigens).

In some embodiments, the provided engineered endothelial cells evade immune recognition. In some embodiments, the engineered endothelial cells described herein, such as primary endothelial cells isolated from one or more individual donors (e.g., healthy donors) or endothelial cells differentiated from iPSCs derived from one or more individual donors (e.g., healthy donors), do not activate an immune response in the patient (e.g., recipient upon administration). Provided are methods of treating a disease by administering a population of engineered endothelial cells described herein to a subject (e.g., recipient) or patient in need thereof.

In some embodiments, the engineered endothelial cells, such as primary endothelial cells isolated from one or more individual donors (e.g., healthy donors) or endothelial cells differentiated from iPSCs derived from one or more individual donors (e.g., healthy donors), are administered to a patient, e.g., a human patient in need thereof. The engineered endothelial cells can be administered to a patient suffering from a disease or condition such as, but not limited to, cardiovascular disease, vascular disease, peripheral vascular disease, ischemic disease, myocardial infarction, congestive heart failure, peripheral vascular obstructive disease, stroke, reperfusion injury, limb ischemia, neuropathy (e.g., peripheral neuropathy or diabetic neuropathy), organ failure (e.g., liver failure, kidney failure, and the like), diabetes, rheumatoid arthritis, osteoporosis, vascular injury, tissue injury, hypertension, angina pectoris and myocardial infarction due to coronary artery disease, renal vascular hypertension, renal failure due to renal artery stenosis, claudication of the lower extremities, and the like. In certain embodiments, the patient has suffered from or is suffering from a transient ischemic attack or stroke, which in some cases, may be due to cerebrovascular disease. In some embodiments, the engineered endothelial cells are administered to treat tissue ischemia e.g., as occurs in atherosclerosis, myocardial infarction, and limb ischemia and to repair of injured blood vessels. In some instances, the cells are used in bioengineering of grafts.

For instance, the engineered endothelial cells can be used in cell therapy for the repair of ischemic tissues, formation of blood vessels and heart valves, engineering of artificial vessels, repair of damaged vessels, and inducing the formation of blood vessels in engineered tissues (e.g., prior to transplantation). Additionally, the endothelial cells can be further modified to deliver agents to target and treat tumors.

In many embodiments, provided herein is a method of repair or replacement for tissue in need of vascular cells or vascularization. The method involves administering to a human patient in need of such treatment, a composition containing the engineered endothelial cells, such as isolated primary endothelial cells or differentiated endothelial cells, to promote vascularization in such tissue. The tissue in need of vascular cells or vascularization can be a cardiac tissue, liver tissue, pancreatic tissue, renal tissue, muscle tissue, neural tissue, bone tissue, among others, which can be a tissue damaged and characterized by excess cell death, a tissue at risk for damage, or an artificially engineered tissue.

In some embodiments, vascular diseases, which may be associated with cardiac diseases or disorders can be treated by administering endothelial cells, such as but not limited to, definitive vascular endothelial cells and endocardial endothelial cells derived as described herein. Such vascular diseases include, but are not limited to, coronary artery disease, cerebrovascular disease, aortic stenosis, aortic aneurysm, peripheral artery disease, atherosclerosis, varicose veins, angiopathy, infarcted area of heart lacking coronary perfusion, non-healing wounds, diabetic or non-diabetic ulcers, or any other disease or disorder in which it is desirable to induce formation of blood vessels.

In certain embodiments, the endothelial cells are used for improving prosthetic implants (e.g., vessels made of synthetic materials such as Dacron and Gortex.) which are used in vascular reconstructive surgery. For example, prosthetic arterial grafts are often used to replace diseased arteries which perfuse vital organs or limbs. In other embodiments, the engineered endothelial cells are used to cover the surface of prosthetic heart valves to decrease the risk of the formation of emboli by making the valve surface less thrombogenic.

The endothelial cells outlined can be transplanted into the patient using well known surgical techniques for grafting tissue and/or isolated cells into a vessel. In some embodiments, the cells are introduced into the patient's heart tissue by injection (e.g., intramyocardial injection, intracoronary injection, trans-endocardial injection, trans-epicardial injection, percutaneous injection), infusion, grafting, and implantation.

Administration (delivery) of the endothelial cells includes, but is not limited to, subcutaneous or parenteral including intravenous, intraarterial (e.g., intracoronary), intramuscular, intraperitoneal, intramyocardial, trans-endocardial, trans-epicardial, intranasal administration as well as intrathecal, and infusion techniques.

As will be appreciated by those in the art, the cells are transplanted using techniques known in the art that depends on both the cell type and the ultimate use of these cells. In some embodiments, the cells provided herein are transplanted either intravenously or by injection at particular locations in the patient. When transplanted at particular locations, the cells may be suspended in a gel matrix to prevent dispersion while they take hold.

Exemplary endothelial cell types include, but are not limited to, a capillary endothelial cell, vascular endothelial cell, aortic endothelial cell, arterial endothelial cell, venous endothelial cell, renal endothelial cell, brain endothelial cell, liver endothelial cell, and the like.

The endothelial cells outlined herein, such as isolated primary endothelial cells or differentiated endothelial cells, can express one or more endothelial cell markers. Non-limiting examples of such markers include VE-cadherin (CD144). ACE (angiotensin-converting enzyme) (CD143), BNH9/BNF13, CD31, CD34, CD54 (ICAM-1), CD62E (E-Selectin), CD105 (Endoglin), CD146, Endocan (ESM-1), Endoglyx-1, Endomucin, Eotaxin-3, EPAS1 (Endothelial PAS domain protein 1), Factor VIII related antigen, FLI-1, Flk-1 (KDR, VEGFR-2), FLT-1 (VEGFR-1), GATA2, GBP-1 (guanylate- binding protein-1). GRO-alpha, HEX, ICAM-2(intercellular adhesion molecule 2), LM02, LYVE-1, MRB(magic roundabout), Nucleolin, PAL-E (pathologische anatomie Leiden- endothelium), RTKs, sVCAM-1, TALI, TEM1 (Tumor endothelial marker 1), TEMS (Tumor endothelial marker 5), TEM7 (Tumor endothelial marker 7), thrombomodulin (TM, CD141), VCAM-1 (vascular cell adhesion molecule- 1) (CD106), VEGF, vWF (von Willebrand factor), ZO-1, endothelial cell-selective adhesion molecule (ESAM), CD102, CD93, CD184, CD304, and DLL4.

In some embodiments, the endothelial cells are further genetically modified to express an exogenous gene encoding a protein of interest such as but not limited to an enzyme, hormone, receptor, ligand, or drug that is useful for treating a disorder/condition or ameliorating symptoms of the disorder/condition. Standard methods for genetically modifying endothelial cells are described, e.g., in U.S. Pat. No. 5,674,722.

Such endothelial cells can be used to provide constitutive synthesis and delivery of polypeptides or proteins, which are useful in prevention or treatment of disease. In this way, the polypeptide is secreted directly into the bloodstream or other area of the body (e.g., central nervous system) of the individual. In some embodiments, the endothelial cells can be modified to secrete insulin, a blood clotting factor (e.g., Factor VIII or von Willebrand Factor), alpha-1 antitrypsin, adenosine deaminase, tissue plasminogen activator, interleukins (e.g., IL-1, IL-2, IL-3), and the like.

In certain embodiments, the endothelial cells can be modified in a way that improves their performance in the context of an implanted graft. Non-limiting illustrative examples include secretion or expression of a thrombolytic agent to prevent intraluminal clot formation, secretion of an inhibitor of smooth muscle proliferation to prevent luminal stenosis due to smooth muscle hypertrophy, and expression and/or secretion of an endothelial cell mitogen or autocrine factor to stimulate endothelial cell proliferation and improve the extent or duration of the endothelial cell lining of the graft lumen.

In some embodiments, the engineered endothelial cells are utilized for delivery of therapeutic levels of a secreted product to a specific organ or limb. For example, a vascular implant lined with endothelial cells engineered (transduced) in vitro can be grafted into a specific organ or limb. The secreted product of the transduced endothelial cells will be delivered in high concentrations to the perfused tissue, thereby achieving a desired effect to a targeted anatomical location.

In other embodiments, the endothelial cells are further genetically modified to contain a gene that disrupts or inhibits angiogenesis when expressed by endothelial cells in a vascularizing tumor. In some cases, the endothelial cells can also be genetically modified to express any one of the selectable suicide genes described herein which allows for negative selection of grafted endothelial cells upon completion of tumor treatment.

In some embodiments, endothelial cells described herein, such as isolated primary endothelial cells or differentiated endothelial cells, are administered to a recipient subject to treat a vascular disorder selected from the group consisting of vascular injury, cardiovascular disease, vascular disease, peripheral vascular disease, ischemic disease, myocardial infarction, congestive heart failure, peripheral vascular obstructive disease, hypertension, ischemic tissue injury, reperfusion injury, limb ischemia, stroke, neuropathy (e.g., peripheral neuropathy or diabetic neuropathy), organ failure (e.g., liver failure, kidney failure, and the like), diabetes, rheumatoid arthritis, osteoporosis, cerebrovascular disease, hypertension, angina pectoris and myocardial infarction due to coronary artery disease, renal vascular hypertension, renal failure due to renal artery stenosis, claudication of the lower extremities, other vascular condition or disease.

### F. Epithelial Cells

Epithelial cells to be used in a cell therapy product may be profiled for donor capability at any stage of the manufacturing process of the cell therapy product.

Epithelial cells used in a cell therapy product may be primary epithelial cells. Methods for profiling a population of cells for donor capability as described anywhere herein may be performed on primary (e.g., genome-edited) epithelial cells.

As described elsewhere herein, epithelial cells used in a cell therapy product may be pluripotent stem cell (iPSC)-derived epithelial cells. Methods Methods for profiling a population of cells for donor capability as described anywhere herein may also be performed on stem cells capable of differentiating to form epithelial cells. Methods for profiling a population of cells for donor capability as described anywhere herein may also be performed on stem cell derived (e.g., genome-edited) epithelial cells.

Relevant information concerning epithelial cells as referred to in the context of the present disclosure is known in the art, including certain information regarding desired features of epithelial cells when used for cell therapy. It will be understood that embodiments concerning epithelial cells described herein may be readily and appropriately combined with embodiments describing HIP cells (e.g., exhibiting reduced expression of one or more molecules of the MHC class I and/or MHC class II molecules and increased expression of at least one tolerogenic factor), as well as embodiments describing safety switches, and other modified/gene edited cells as described herein. Epithelial cells to be used in a cell therapy product may be profiled for donor capability at any stage of the editing process during manufacturing of the cell therapy product.

The epithelial cells described herein may be used to treat or prevent a disease in a subject.

### 1) Retinal Pigmented Epithelium (RPE) Cells

Relevant information concerning RPE cells as referred to in the context of the present disclosure is known in the art, including certain information regarding desired features of RPE cells when used for cell therapy. In some embodiments, the cells that are engineered or modified as provided herein are primary retinal pigmented epithelium (RPE) cells. In some embodiments, the primary RPE cells are isolated or obtained from one or more individual donor subjects, such as one or more individual healthy donor (e.g., a subject that is not known or suspected of, e.g., not exhibiting clinical signs of, a disease or infection). As will be appreciated by those in the art, methods of isolating or obtaining RPE cells from an individual can be achieved using known techniques. Provided herein are engineered primary RPE cells that contain modifications (e.g., genetic modifications) described herein for subsequent transplantation or engraftment into subjects (e.g., recipients).

In some embodiments, primary RPE cells are obtained (e.g., harvested, extracted, removed, or taken) from a subject or an individual. In some embodiments, primary RPE cells are produced from a pool of RPE cells such that the RPE cells are from one or more subjects (e.g., one or more human including one or more healthy humans). In some embodiments, the pool of primary RPE cells is from 1-100, 1-50, 1-20. 1-10, 1 or more, 2 or more, 3 or more, 4 or more, 5 or more, 10 or more, 20 or more, 30 or more, 40 or more, 50 or more, or 100 or more subjects. In some embodiments, the donor subject is different from the patient (e.g., the recipient that is administered the therapeutic cells). In some embodiments, the pool of RPE cells does not include cells from the patient. In some embodiments, one or more of the donor subjects from which the pool of RPE cells is obtained are different from the patient.

In some embodiments, the cells as provided herein are RPE cells differentiated from engineered iPSCs that contain modifications (e.g., genetic modifications) described herein and that are differentiated into a RPE cell. As will be appreciated by those in the art, the methods for differentiation depend on the desired cell type using known techniques. In some embodiments, the cells differentiated into a RPE cell may be used for subsequent transplantation or engraftment into subjects (e.g., recipients).

Useful methods for differentiating pluripotent stem cells into RPE cells are described in, for example, U.S. Pat. No. 9,458,428 and US9.850,463, the disclosures are herein incorporated by reference in their entirety, including the specifications. Additional methods for producing RPE cells from human induced pluripotent stem cells can be found in, for example, Lamba et al., PNAS, 2006, 103(34): 12769-12774; Mellough et al, Stem Cells, 2012, 30(4):673-686: Idelson et al, Cell Stem Cell, 2009. 5(4): 396-408: Rowland et al. Journal of Cellular Physiology. 2012. 227(2):457-466, Buchholz et al, Stem Cells Trans Med. 2013.2(5): 384-393, and da Cruz et al, Nat Biotech, 2018, 36:328-337.

Human pluripotent stem cells have been differentiated into RPE cells using the techniques outlined in Kamao et al, Stem Cell Reports 2014:2:205-18, hereby incorporated by reference in its entirety and in particular for the methods and reagents outlined there for the differentiation techniques and reagents; see also Mandai et al., N Engl J Med, 2017, 376:1038-1046, the contents herein incorporated in its entirety for techniques for generating sheets of RPE cells and transplantation into patients. Differentiation can be assayed as is known in the art, generally by evaluating the presence of RPE associated and/or specific markers or by measuring functionally. See for example Kamao et al., Stem Cell Reports, 2014. 2(2):205-18, the contents incorporated herein by reference in its entirety and specifically for the markers outlined in the first paragraph of the results section.

In some embodiments, the method of producing a population of engineered retinal pigmented epithelium (RPE) cells from a population of engineered pluripotent cells by in vitro differentiation comprises: (a) culturing the population of engineered pluripotent cells in a first culture medium comprising any one of the factors selected from the group consisting of activin A, bFGF, BMP4/7, DKK1, IGF1, noggin, a BMP inhibitor, an ALK inhibitor, a ROCK inhibitor, and a VEGFR inhibitor to produce a population of pre-RPE cells; and (b) culturing the population of pre-RPE cells in a second culture medium that is different than the first culture medium to produce a population of engineered RPE cells. In some embodiments, the ALK inhibitor is SB-431542, a derivative thereof, or a variant thereof. In some instances, the ALK inhibitor is at a concentration ranging from about 2 mM to about 10 pM. In some embodiments, the ROCK inhibitor is Y-27632, a derivative thereof, or a variant thereof. In some instances, the ROCK inhibitor is at a concentration ranging from about 1 pM to about 10 pM. In some embodiments, the first culture medium and/or second culture medium are absent of animal serum.

Differentiation can be assayed as is known in the art, generally by evaluating the presence of RPE associated and/or specific markers or by measuring functionally. See for example Kamao et al., Stem Cell Reports, 2014, 2(2):205-18, the contents are herein incorporated by reference in its entirety and specifically for the results section.

Additional descriptions of RPE cells, including methods for their differentiation and for use in the present technology, are found in WO2020/018615, the disclosure of which is herein incorporated by reference in its entirety.

In some embodiments, the population of engineered RPE cells, such as primary RPE cells isolated from one or more individual donors (e.g., healthy donors) or RPE cells differentiated from iPSCs derived from one or more individual donors (e.g., healthy donors), are maintained in culture, in some cases expanded, prior to administration. In certain embodiments, the population of RPE cells are cryopreserved prior to administration.

Exemplary RPE cell types include, but are not limited to, retinal pigmented epithelium (RPE) cell, RPE progenitor cell, immature RPE cell, mature RPE cell, functional RPE cell, and the like.

In some embodiments, the RPE cells, such as primary RPE cells isolated from one or more individual donors (e.g., healthy donors) or RPE cells differentiated from iPSCs derived from one or more individual donors (e.g., healthy donors), have a genetic expression profile similar or substantially similar to that of native RPE cells. Such RPE cells may possess the polygonal, planar sheet morphology of native RPE cells when grown to confluence on a planar substrate.

In some embodiments, the present technology is directed to engineered RPE cells, such as primary RPE cells isolated from one or more individual donors (e.g., healthy donors) or RPE cells differentiated from iPSCs derived from one or more individual donors (e.g., healthy donors), that overexpress a tolerogenic factor (e.g., CD47), have reduced expression or lack expression of one or more MHC class I molecules and/or one or more MHC class II molecules (e.g., one or more MHC class I human leukocyte antigens and/or one or more MHC class II human leukocyte antigens).

In some embodiments, the provided engineered RPE cells evade immune recognition. In some embodiments, the engineered RPE cells described herein, such as primary RPE cells isolated from one or more individual donors (e.g., healthy donors) or RPE cells differentiated from iPSCs derived from one or more individual donors (e.g., healthy donors), do not activate an immune response in the patient (e.g., recipient upon administration). Provided are methods of treating a disease by administering a population of engineered RPE cells described herein to a subject (e.g., recipient) or patient in need thereof.

The RPE cells can be implanted into a patient suffering from macular degeneration or a patient having damaged RPE cells. In some embodiments, the patient has age-related macular degeneration (AMD), early AMD, intermediate AMD, late AMD, non-neovascular age-related macular degeneration, dry macular degeneration (dry age-related macular degeneration), wet macular degeneration (wet age-real ted macular degeneration), juvenile macular degeneration (JMD) (e.g., Stargardt disease, Best disease, and juvenile retinoschisis), Leber's Congenital Ameurosis, or retinitis pigmentosa. In other embodiments, the patient suffers from retinal detachment.

For therapeutic application, cells prepared according to the disclosed methods can typically be supplied in the form of a pharmaceutical composition comprising an isotonic excipient, and are prepared under conditions that are sufficiently sterile for human administration. For general principles in medicinal formulation of cell compositions, see “Cell Therapy: Stem Cell Transplantation, Gene Therapy, and Cellular Immunotherapy,” by Morstyn & Sheridan eds, Cambridge University Press, 1996; and “Hematopoietic Stem Cell Therapy,” E. D. Ball, J. Lister & P. Law, Churchill Livingstone, 2000. The cells can be packaged in a device or container suitable for distribution or clinical use.

### 2) Thyroid Cells

Relevant information concerning thyroid cells as referred to in the context of the present disclosure is known in the art, including certain information regarding desired features of thyroid cells when used for cell therapy. In some embodiments, the cells that are engineered or modified as provided herein are primary thyroid cells. In some embodiments, the primary thyroid cells are isolated or obtained from one or more individual donor subjects, such as one or more individual healthy donor (e.g., a subject that is not known or suspected of, e.g., not exhibiting clinical signs of, a disease or infection). As will be appreciated by those in the art, methods of isolating or obtaining thyroid cells from an individual can be achieved using known techniques. Provided herein are engineered primary thyroid cells that contain modifications (e.g., genetic modifications) described herein for subsequent transplantation or engraftment into subjects (e.g., recipients).

In some embodiments, primary thyroid cells are obtained (e.g., harvested, extracted, removed, or taken) from a subject or an individual. In some embodiments, primary thyroid cells are produced from a pool of thyroid cells such that the thyroid cells are from one or more subjects (e.g., one or more human including one or more healthy humans). In some embodiments, the pool of primary thyroid cells is from 1-100, 1-50, 1-20. 1-10. 1 or more, 2 or more, 3 or more, 4 or more, 5 or more, 10 or more, 20 or more, 30 or more, 40 or more, 50 or more, or 100 or more subjects. In some embodiments, the donor subject is different from the patient (e.g., the recipient that is administered the therapeutic cells). In some embodiments, the pool of thyroid cells does not include cells from the patient. In some embodiments, one or more of the donor subjects from which the pool of thyroid cells is obtained are different from the patient.

In some embodiments, the cells as provided herein are thyroid cells differentiated from engineered iPSCs that contain modifications (e.g., genetic modifications) described herein and that are differentiated into a thyroid cell. As will be appreciated by those in the art, the methods for differentiation depend on the desired cell type using known techniques. In some embodiments, the cells differentiated into a thyroid cell may be used for subsequent transplantation or engraftment into subjects (e.g., recipients).

In some embodiments, engineered pluripotent cells containing modifications described herein are differentiated into thyroid progenitor cells and thyroid follicular organoids that can secrete thyroid hormones to address autoimmune thyroiditis. Techniques to differentiate thyroid cells are known the art. See, e.g., Kurmann et al., Cell Stem Cell, 2015 Nov. 5:17(5):527-42, incorporated herein by reference in its entirety and specifically for the methods and reagents for the generation of thyroid cells from human pluripotent stem cells, and also for transplantation techniques. Differentiation can be assayed as is known in the art, generally by evaluating the presence of thyroid cell associated or specific markers or by measuring functionally.

In some embodiments, the population of engineered thyroid cells, such as primary thyroid cells isolated from one or more individual donors (e.g., healthy donors) or thyroid cells differentiated from iPSCs derived from one or more individual donors (e.g., healthy donors), are maintained in culture, in some cases expanded, prior to administration. In certain embodiments, the population of thyroid cells are cryopreserved prior to administration.

In some embodiments, the present technology is directed to engineered thyroid cells, such as primary thyroid cells isolated from one or more individual donors (e.g., healthy donors) or thyroid cells differentiated from iPSCs derived from one or more individual donors (e.g., healthy donors), that overexpress a tolerogenic factor (e.g., CD47), and have reduced expression or lack expression of one or more MHC class I molecules and/or one or more MHC class II molecules (e.g., one or more MHC class I human leukocyte antigens and/or one or more MHC class II human leukocyte antigens).

In some embodiments, the provided engineered thyroid cells evade immune recognition. In some embodiments, the engineered thyroid cells described herein, such as primary thyroid cells isolated from one or more individual donors (e.g., healthy donors) or beta islet cells differentiated from iPSCs derived from one or more individual donors (e.g., healthy donors), do not activate an immune response in the patient (e.g., recipient upon administration). Provided are methods of treating a disease by administering a population of engineered endothelial cells described herein to a subject (e.g., recipient) or patient in need thereof.

### G. Cardiac Cells

Cardiac cells to be used in a cell therapy product may be profiled for donor capability at any stage of the manufacturing process of the cell therapy product.

Cardiac cells used in a cell therapy product may be primary cardiac cells. Methods for profiling a population of cells for donor capability as described anywhere herein may be performed on primary (e.g., genome-edited) cardiac cells.

As described elsewhere herein, cardiac cells used in a cell therapy product may be pluripotent stem cell (iPSC)-derived cardiac cells. Methods for profiling a population of cells for donor capability as described anywhere herein may also be performed on stem cells capable of differentiating to form cardiac cells. Methods for profiling a population of cells for donor capability as described anywhere herein may also be performed on stem cell derived (e.g., genome-edited) cardiac cells.

Relevant information conceding cardiac cells as referred to in the context of the present disclosure is known in the art, including certain information regarding desired features of cardiac cells when used for cell therapy. It will be understood that embodiments concerning cardiac cells described herein may be readily and appropriately combined with embodiments describing HIP cells (e.g., exhibiting reduced expression of one or more molecules of the MHC class I and/or MHC class II molecules and increased expression of at least one tolerogenic factor), as well as embodiments describing safety switches, and other modified/gene edited cells as described herein. Cardiac cells to be used in a cell therapy product may be profiled for donor capability at any stage of of the editing process during manufacturing of the cell therapy product.

The cardiac cells described herein may be used to treat or prevent a disease in a subject.

Provided herein are cardiac cell types differentiated from HIP cells for subsequent transplantation or engraftment into subjects (e.g., recipients). As will be appreciated by those in the art, the methods for differentiation depend on the desired cell type using known techniques. Exemplary cardiac cell types include, but are not limited to, a cardiomyocyte, nodal cardiomyocyte, conducting cardiomyocyte, working cardiomyocyte, cardiomyocyte precursor cell, cardiomyocyte progenitor cell, cardiac stem cell, cardiac muscle cell, atrial cardiac stem cell, ventricular cardiac stem cell, epicardial cell, hematopoietic cell, vascular endothelial cell, endocardial endothelial cell, cardiac valve interstitial cell, cardiac pacemaker cell, and the like.

In some embodiments, cardiac cells described herein are administered to a recipient subject to treat a cardiac disorder selected from the group consisting of pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, idiopathic cardiomyopathy, other cardiomyopathy, myocardial ischemic reperfusion injury, ventricular dysfunction, heart failure, congestive heart failure, coronary artery disease, end-stage heart disease, atherosclerosis, ischemia, hypertension, restenosis, angina pectoris, rheumatic heart, arterial inflammation, cardiovascular disease, myocardial infarction, myocardial ischemia, congestive heart failure, myocardial infarction, cardiac ischemia, cardiac injury, myocardial ischemia, vascular disease, acquired heart disease, congenital heart disease, atherosclerosis, coronary artery disease, dysfunctional conduction systems, dysfunctional coronary arteries, pulmonary hypertension, cardiac arrhythmias, muscular dystrophy, muscle mass abnormality, muscle degeneration, myocarditis, infective myocarditis, drug- or toxin-induced muscle abnormalities, hypersensitivity myocarditis, and autoimmune endocarditis.

Accordingly, provided herein are methods for the treatment and prevention of a cardiac injury or a cardiac disease or disorder in a subject in need thereof. The methods described herein can be used to treat, ameliorate, prevent or slow the progression of a number of cardiac diseases or their symptoms, such as those resulting in pathological damage to the structure and/or function of the heart. The terms “cardiac disease,” “cardiac disorder,” and “cardiac injury,” are used interchangeably herein and refer to a condition and/or disorder relating to the heart, including the valves, endothelium, infarcted zones, or other components or structures of the heart. Such cardiac diseases or cardiac-related disease include, but are not limited to, myocardial infarction, heart failure, cardiomyopathy, congenital heart defect, heart valve disease or dysfunction, endocarditis, rheumatic fever, mitral valve prolapse, infective endocarditis, hypertrophic cardiomyopathy, dilated cardiomyopathy, myocarditis, cardiomegaly, and/or mitral insufficiency, among others.

In some embodiments, the cardiomyocyte precursor includes a cell that is capable giving rise to progeny that include mature (end-stage) cardiomyocytes. Cardiomyocyte precursor cells can often be identified using one or more markers selected from GATA-4, Nkx2.5, and the MEF-2 family of transcription factors. In some instances, cardiomyocytes refer to immature cardiomyocytes or mature cardiomyocytes that express one or more markers (sometimes at least 2, 3, 4 or 5 markers) from the following list: cardiac troponin I (cTnl), cardiac troponin T (cTnT), sarcomeric myosin heavy chain (MHC), GATA-4, Nkx2.5, N-cadherin, β2-adrenoceptor, ANF, the MEF-2 family of transcription factors, creatine kinase MB (CK-MB), nwoglobin, and atrial natriuretic factor (ANF). In some embodiments, the cardiac cells demonstrate spontaneous periodic contractile activity. In some cases, when that cardiac cells are cultured in a suitable tissue culture environment with an appropriate Ca²⁺ concentration and electrolyte balance, the cells can be observed to contract in a periodic fashion across one axis of the cell, and then release from contraction, without having to add any additional components to the culture medium. In some embodiments, the cardiac cells are hypoimmunogenic cardiac cells.

In some embodiments, the method of producing a population of hypoimmunogenic cardiac cells from a population of hypoimmunogenic pluripotent (HIP) cells by in vitro differentiation comprises: (a) culturing a population of HIP cells in a culture medium comprising a GSK inhibitor; (b) culturing the population of HIP cells in a culture medium comprising a WNT antagonist to produce a population of pre-cardiac cells; and (c) culturing the population of pre-cardiac cells in a culture medium comprising insulin to produce a population of hypoimmune cardiac cells. In some embodiments, the GSK inhibitor is CHIR-99021, a derivative thereof, or a variant thereof. In some instances, the GSK inhibitor is at a concentration ranging from about 2 mM to about 10 mM. In some embodiments, the WNT antagonist is IWR1, a derivative thereof, or a variant thereof. In some instances, the WNT antagonist is at a concentration ranging from about 2 mM to about 10 mM.

In some embodiments, the population of hypoimmunogenic cardiac cells is isolated from non-cardiac cells. In some embodiments, the isolated population of hypoimmunogenic cardiac cells are expanded prior to administration. In certain embodiments, the isolated population of hypoimmunogenic cardiac cells are expanded and cryopreserved prior to administration.

Other useful methods for differentiating induced pluripotent stem cells or pluripotent stem cells into cardiac cells are described, for example, in US2017/0152485; US2017/0058263; US2017/0002325; US2016/0362661; US2016/0068814; U.S. Pat. No. 9,062,289; US7,897.389; and U.S. Pat. No. 7,452,718. Additional methods for producing cardiac cells from induced pluripotent stem cells or pluripotent stem cells are described in, for example, Xu et al, Stem Cells and Development, 2006. 15(5): 631-9, Burridge et al, Cell Stem Cell, 2012, 10: 16-28, and Chen et al. Stem Cell Res. 2015, 15(2):365-375.

In various embodiments, hypoimmunogenic cardiac cells can be cultured in culture medium comprising a BMP pathway inhibitor, a WNT signaling activator, a WNT signaling inhibitor, a WNT agonist, a WNT antagonist, a Src inhibitor, a EGFR inhibitor, a PCK activator, a cytokine, a growth factor, a cardiotropic agent, a compound, and the like.

The WNT signaling activator includes, but is not limited to, CHIR99021. The PCK activator includes, but is not limited to, PMA. The WNT signaling inhibitor includes, but is not limited to, a compound selected from KY02111, S03031 (KY01-I), S02031 (KY02-I), and S03042 (KY03-I), and XAV939. The Src inhibitor includes, but is not limited to, A419259. The EGFR inhibitor includes, but is not limited to, AG1478.

Non-limiting examples of an agent for generating a cardiac cell from an iPSC include activin A, BMP4, Wnt3a, VEGF, soluble frizzled protein, cyclosporin A, angiotensin II, phenylephrine, ascorbic acid, dimethylsulfoxide, 5-aza-2′-deoxycytidine, and the like.

The cells provided herein can be cultured on a surface, such as a synthetic surface to support and/or promote differentiation of hypoimmunogenic pluripotent cells into cardiac cells. In some embodiments, the surface comprises a polymer material including, but not limited to, a homopolymer or copolymer of selected one or more acrylate monomers. Non-limiting examples of acrylate monomers and methacrylate monomers include tetra(ethylene glycol) diacrylate, glycerol dimethacrylate, 1,4-butanediol dimethacrylate, poly(ethylene glycol) diacrylate, di(ethylene glycol) dimethacrylate, tetra(ethyiene glycol) dimethacrylate, 1,6-hexanediol propoxylate diacrylate, neopentyl glycol diacrylate, trimethylolpropane benzoate diacrylate, trimethylolpropane eihoxylate (1 EO/QH) methyl, tricyclo[5.2.1.0^{2,6}] decane dimethanol diacrylate, neopentyl glycol exhoxylate diacrylate, and trimethylolpropane triacrylate. Acrylate synthesized as known in the art or obtained from a commercial vendor, such as Polysciences, Inc., Sigma Aldrich, Inc. and Sartomer, Inc.

The polymeric material can be dispersed on the surface of a support material. Useful support materials suitable for culturing cells include a ceramic substance, a glass, a plastic, a polymer or co-polymer, any combinations thereof, or a coating of one material on another. In some instances, a glass includes soda-lime glass, pyrex glass, vycor glass, quartz glass, silicon, or derivatives of these or the like.

In some instances, plastics or polymers including dendritic polymers include poly(vinyl chloride), poly(vinyl alcohol), poly(methyl methacrylate), poly(vinyl acetate-maleic anhydride), poly(dimethylsiloxane) monomethacrylate, cyclic olefin polymers, fluorocarbon polymers, polystyrenes, polypropylene, polyethyleneimine or derivatives of these or the like. In some instances, copolymers include poly(vinyl acetate-co-maleic anhydride), poly(styrene-co-maleic anhydride), poly(ethylene-co-acrylic acid) or derivatives of these or the like.

The efficacy of cardiac cells prepared as described herein can be assessed in animal models for cardiac cryoinjury, which causes 55% of the left ventricular wall tissue to become scar tissue without treatment (Li et al, Ann. Thorac. Surg. 62:654, 1996: Sakai et al. Ann. Thorac. Surg. 8:2074, 1999, Sakai et al., Thorac. Cardiovasc. Surg. 118:715, 1999). Successful treatment can reduce the area of the scar, limit scar expansion, and improve heart function as determined by systolic, diastolic, and developed pressure. Cardiac injury can also be modeled using an embolization coil in the distal portion of the left anterior descending artery (Watanabe et al., Cell Transplant. 7:239, 1998), and efficacy of treatment can be evaluated by histology and cardiac function.

In some embodiments, the population of engineered cardiac cells, such cardiac cells differentiated from iPSCs derived from one or more individual donors (e.g., healthy donors), are maintained in culture, in some cases expanded, prior to administration. In certain embodiments, the population of cardiac cells are cryopreserved prior to administration.

In some embodiments, the present technology is directed to engineered cardiac cells, such as cardiac cells differentiated from iPSCs derived from one or more individual donors (e.g., healthy donors), that overexpress a tolerogenic factor (e.g., CD47), have reduced expression or lack expression of one or more MHC class I molecules and/or one or more MHC class II molecules (e.g., one or more MHC class I human leukocyte antigens and/or one or more MHC class II human leukocyte antigens).

In some embodiments, the provided engineered cardiac cells evade immune recognition. In some embodiments, the engineered cardiac cells described herein, such as cardiac cells differentiated from iPSCs derived from one or more individual donors (e.g., healthy donors), do not activate an immune response in the patient (e.g., recipient upon administration). Provided are methods of treating a disease by administering a population of engineered cardiac cells described herein to a subject (e.g., recipient) or patient in need thereof.

In some embodiments, the administration comprises implantation into the subject's heart tissue, intravenous injection, intraarterial injection, intracoronary injection, intramuscular injection, intraperitoneal injection, intramyocardial injection, trans-endocardial injection, trans-epicardial injection, or infusion.

In some embodiments, the patient administered the engineered cardiac cells is also administered a cardiac drug. Illustrative examples of cardiac drugs that are suitable for use in combination therapy include, but are not limited to, growth factors, polynucleotides encoding growth factors, angiogenic agents, calcium channel blockers, antihypertensive agents, antimitotic agents, inotropic agents, anti-atherogenic agents, anti-coagulants, beta-blockers, anti-arhythmic agents, anti-inflammatory agents, vasodilators, thrombolytic agents, cardiac glycosides, antibiotics, antiviral agents, antifungal agents, agents that inhibit protozoans, nitrates, angiotensin converting enzyme (ACE) inhibitors, angiotensin II receptor antagonist, brain natriuretic peptide (BNP); antineoplastic agents, steroids, and the like.

The effects of therapy according to the methods provided herein can be monitored in a variety of ways. For instance, an electrocardiogram (ECG) or holier monitor can be utilized to determine the efficacy of treatment. An ECG is a measure of the heart rhythms and electrical impulses, and is a very effective and non-invasive way to determine if therapy has improved or maintained, prevented, or slowed degradation of the electrical conduction in a subject's heart. The use of a holier monitor, a portable ECG that can be worn for long periods of time to monitor heart abnormalities, arrhythmia disorders, and the like, is also a reliable method to assess the effectiveness of therapy. An ECG or nuclear study can be used to determine improvement in ventricular function.

### 1) Cardiomyocytes

Cardiomyocytes to be used in a cell therapy product may be profiled for donor capability at any stage of the manufacturing process of the cell therapy product.

Cardiomyocytes used in a cell therapy product may be primary cardiomyocytes. Methods for profiling a population of cells for donor capability as described anywhere herein may be performed on primary (e.g., genome-edited) cardiomyocytes.

As described elsewhere herein, cardiomyocytes used in a cell therapy product may be pluripotent stem cell (iPSC)-derived cardiomyocytes. Methods for profiling a population of cells for donor capability as described anywhere herein may also be performed on stem cells capable of differentiating to form cardiomyocytes. Methods for profiling a population of cells for donor capability as described anywhere herein may also be performed on stem cell derived (e.g., genome-edited) cardiomyocytes.

Relevant information concerning cardiomyocytes as referred to in the context of the present disclosure is known in the art, including certain information regarding desired features of cardiomyocytes when used for cell therapy. It will be understood that embodiments concerning cardiomyocytes described herein may be readily and appropriately combined with embodiments describing HIP cells (e.g., exhibiting reduced expression of one or more molecules of the MHC class I and/or MHC class II molecules and increased expression of at least one tolerogenic factor), as well as embodiments describing safety switches, and other modified/gene edited cells as described herein. Cardiomyocytes to be used in a cell therapy product may be profiled for donor capability at any stage of the editing process during manufacturing of the cell therapy product.

The cardiomyocytes described herein may be used to treat or prevent a disease in a subject.

Cells disclosed herein may be cardiomyocytes, for example a population of cardiomyocytes may be used. It will be understood that any reference to “a cell” e.g. “a cardiomyocyte” below also applies to “a population of cells” e.g. “a population of cardiomyocytes” as described in the present application.

Cardiomyocytes typically express the proteins NKX2.5, cTNT, ACTN2, TNNI1, TNNI3, MYH6, MYH7, MYL2, and MYL7. These proteins may therefore be used as markers of cardiomyocytes.

In some embodiments, the one or more cardiomyocyte markers comprises one or more markers selected from the group consisting of NKX2.5, cTNT, ACTN2, TNNI1, TNNI3, MYH6, MYH7, MYL2, and MYL7. In some embodiments, the one or more cardiomyocyte markers comprise MYH6 and MYL7. In some embodiments, the one or more cardiomyocyte markers comprise NKX2.5, cTNT, MYH6, and MYL7. In some embodiments, the one or more cardiomyocyte markers comprise NKX2.5 and cTNT.

The cardiomyocytes may be produced by any methods known in the art.

In some embodiments, the cardiomyocytes may be differentiating from pluripotent stem cells, wherein the population has a frequency of the presence of one or more cardiomyocyte markers that is or is at least 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, or 98% on or about any one of days 8 to 22, and wherein the differentiating is initiated on day 0.

In some embodiments, the cardiomyocytes may be differentiated from pluripotent stem cells, wherein the population has a frequency of the presence of one or more cardiomyocyte markers that is or is at least 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, or 98% on or about any one of days 8 to 22, and wherein the differentiation is initiated on day 0.

In some embodiments, the differentiating is initiated on the first day that the pluripotent stem cells are cultured in a media comprising an inhibitor of glycogen synthase kinase 3 (GSK3)/activator of Wnt/β-catenin signaling.

In some embodiments, the one or more cardiomyocyte markers comprises one or more markers selected from the group consisting of NKX2.5, cTNT, ACTN2, TNNI1, TNNI3, MYH6. MYH7, MYL2, and MYL7. In some embodiments, the one or more cardiomyocyte markers comprise MYH6 and MYL7. In some embodiments, the one or more cardiomyocyte markers comprise NKX2.5, cTNT, MYH6, and MYL7. In some embodiments, the one or more cardiomyocyte markers comprise NKX2.5 and cTNT.

In some embodiments, the population has a frequency of NKX2.5+/cTNT+ cells that is or is at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98% on or about day 8, 9, 10, 11, or 12. In some embodiments, the population has a frequency of NKX2.5+/cTNT+ cells that is or is at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98% on or about day 8, 9, 10, 11, or 12. In some embodiments, the population has a frequency of MYH6+/MYL7+ cells that is or is at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98% on or about day 8, 9, 10, 11, or 12. In some embodiments, the population has a frequency of NKX2.5+/cTNT+/MYH6+/MYL7+ cells that is or is at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98% on or about day 8, 9, 10, 11, or 12.

In some embodiments, the population has a frequency of NKX2.5+/cTNT+ cells that is or is at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98% on or about day 20, 21, or 22. In some embodiments, the population has a frequency of NKX2.5+/cTNT+ cells that is or is at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98% on or about day 20, 21, or 22. In some embodiments, the population has a frequency of MYH6+/MYL7+ cells that is or is at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98% on or about day 20, 21, or 22. In some embodiments, the population has a frequency of NKX2.5+/cTNT+/MYH6+/MYL7+ cells that is or is at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98% on or about day 20, 21, or 22.

In some embodiments, the population has a frequency of NKX2.5+/cTNT+ cells, or MYH6+/MYL7+ cells, or NKX2.5+/cTNT+/MYH6+/MYL7+ cells, that is or is at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98% on or about day 9, 10, or 11. In some embodiments, the population has a frequency of NKX2.5+/cTNT+ cells, or MYH6+/MYL7+ cells, or NKX2.5+/cTNT+/MYH6+/MYL7+ cells, that is or is at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98% on or about day 10.

In some embodiments, the population has a frequency of NKX2.5+/cTNT+ cells, or MYH6+/MYL7+ cells, or NKX2.5+/cTNT+/MYH6+/MYL7+ cells, that is or is at least 85%, 86%, 87%, 88%, 89%, 90%, 91%, 92%, 93%, 94%, 95%, 96%, 97%, or 98% on or about day 8, 9, 10, 11, or 12. In some embodiments, the population has a frequency of NKX2.5+/cTNT+ cells, or MYH6+/MYL7+ cells, or NKX2.5+/cTNT+/MYH6+/MYL7+ cells, that is or is at least 85%, 86%, 87%, 88%, 89%, 90%, 91%, 92%, 93%, 94%, 95%, 96%, 97%, or 98% on or about day 9, 10, or 11. In some embodiments, the population has a frequency of NKX2.5+/cTNT+ cells, or MYH6+/MYL7+ cells, or NKX2.5+/cTNT+/MYH6+/MYL7+ cells, that is or is at least 85%, 86%, 87%, 88%, 89%, 90%, 91%, 92%, 93%, 94%, 95%, 96%, 97%, or 98% on or about day 10. In some embodiments, the population has a frequency of NKX2.5+/cTNT+ cells, or MYH6+/MYL7+ cells, or NKX2.5+/cTNT+/MYH6+/MYL7+ cells, that is or is at least 90%, 91%, 92%, 93%, 94%, 95%, 96%, 97%, or 98% on or about day 8, 9, 10, 11, or 12. In some embodiments, the population has a frequency of NKX2.5+/cTNT+ cells, or MYH6+/MYL7+ cells, or NKX2.5+/cTNT+/MYH6+/MYL7+ cells, that is or is at least 85%, 86%, 87%, 88%, 89%, 90%, 91%, 92%, 93%, 94%, 95%, 96%, 97%, or 98% on or about day 9, 10, or 11. In some embodiments, the population has a frequency of NKX2.5+/cTNT+ cells, or MYH6+/MYL7+ cells, or NKX2.5+/cTNT+/MYH6+/MYL7+ cells, that is or is at least 85%, 86%, 87%, 88%, 89%, 90%, 91%, 92%, 93%, 94%, 95%, 96%, 97%, or 98% on or about day 10.

In some embodiments, the pluripotent stem cells are selected from the group consisting of induced pluripotent stem cells, embryonic stem cells, bone marrow-mesenchymal stem cells, cardiac tissue stem cells, and adipose tissue stem cells. In some embodiments, the pluripotent stem cells are induced pluripotent stem cells.

In some embodiments, the population of cardiomyocytes was produced using any one or more of the methods, incubations, e.g., first incubation, second incubation, and/or third incubation, and dissociation by contacting, e.g., contacting with a dissociating agent, as described herein.

In some embodiments, the population of cardiomyocytes was contacted with a dissociating agent on or about day 2, 3, 4, 5, or 6. In some embodiments, the population of cardiomyocytes was contacted with a dissociating agent on or about day 2, 3, or 4. In some embodiments, the population of cardiomyocytes was contacted with a dissociating agent on or about day 4.

In some embodiments, the dissociating agent is or comprises a cleavage enzyme. In some embodiments, the cleavage enzyme is a protease. In some embodiments, the protease is a recombinant enzyme that cleaves a peptide bond on the C-terminal side of a lysine or arginine residue. In some embodiments, the protease is an endopeptidase. In some embodiments, the endopeptidase is trypsin. In some embodiments, the protease is selected from the group consisting of trypsin, collagenase, chymotrypsin, elastase, hyaluronidase, papin, and dispase. In some embodiments, the collagenase is collagenase type I, collagenase type II, or collagenase type III. In some embodiments, the collagenase is collagenase type I, collagenase type II, or collagenase type III. In some embodiments, the protease is collagenase. In some embodiments, the protease is hyaluronidase.

In some embodiments, the population of cardiomyocytes is at or about any one of days 8-22. In some embodiments, the population of cardiomyocytes is at or about day 8, 9, 10, 11, 12, 13, 14, 15, 16, 17, 18, 19, 20, 21, or 22. In some embodiments, the population of cardiomyocytes is at or about day 8, 9, 10, 11, 12, 13, 14, or 15. In some embodiments, the population of cardiomyocytes is at or about day 8, 9, 10, 11, or 12. In some embodiments, the population of cardiomyocytes is at or about day 9, 10, or 11. In some embodiments, the population of cardiomyocytes is at or about day 10.

In some embodiments, the population of cardiomyocytes was harvested at or about any one of days 8-22. In some embodiments, the population of cardiomyocytes was harvested at or about day 8, 9, 10, 11, 12, 13, 14, 15, 16, 17, 18, 19, 20, 21, or 22. In some embodiments, the population of cardiomyocytes was harvested at or about day 8, 9, 10, 11, 12, 13, 14, or 15. In some embodiments, the population of cardiomyocytes was harvested at or about day 8, 9, 10, 11, or 12. In some embodiments, the population of cardiomyocytes was harvested at or about day 9, 10, or 11. In some embodiments, the population of cardiomyocytes was harvested at or about day 10.

In some embodiments, the population of cardiomyocytes has a frequency of NKX2.5+/cTNT+ cells, or MYH6+/MYL7+ cells, or NKX2.5+/cTNT+/MYH6+/MYL7+ cells, that is or is at least 75%, 76%, 77%, 78%, 79%, 80%, 81%, 82%, 83%, 84%, 85%, 86%, 87%, 88%, 89%, 90%,91%, 92%, 93%, 94%, 95%, 96%, 97%, or 98%. In some embodiments, the population of differentiating cardiomyocytes has a frequency of NKX2.5+/cTNT+ cells, or MYH6+/MYL7+ cells, or NKX2.5+/cTNT+/MYH6+/MYL7+ cells, that is or is at least 80%, 81%, 82%, 83%, 84%, 85%, 86%, 87%, 88%, 89%, 90%, 91%, 92%, 93%, 94%, 95%, 96%, 97%, or 98%. In some embodiments, the population of differentiating cardiomyocytes has a frequency of NKX2.5+/cTNT+ cells, or MYH6+/MYL7+ cells, or NKX2.5+/cTNT+/MYH6+/MYL7+ cells, that is or is at least 85%, 86%, 87%, 88%, 89%, 90%, 91%, 92%, 93%, 94%, 95%, 96%, 97%, or 98%. In some embodiments, the population of differentiating cardiomyocytes has a frequency of NKX2.5+/cTNT+ cells, or MYH6+/MYL7+ cells, or NKX2.5+/cTNT+/MYH6+/MYL7+ cells, that is or is at least 90%, 91%, 92%, 93%, 94%, 95%, 96%, 97%, or 98%.

In some embodiments, the population of cardiomyocytes has a frequency of NKX2.5+/cTNT+ cells, or MYH6+/MYL7+ cells, or NKX2.5+/cTNT+/MYH6+/MYL7+ cells, that is or is at least 90%, 91%, 92%, 93%, 94%, 95%, 96%, 97%, or 98%. In some embodiments, the population of differentiating cardiomyocytes has a frequency of NKX2.5+/cTNT+ cells, or MYH6+/MYL7+ cells, or NKX2.5+/cTNT+/MYH6+/MYL7+ cells, that is or is at least 90%, 91%, 92%, 93%, 94%, 95%, 96%, 97%, or 98%. In some embodiments, the population of differentiating cardiomyocytes has a frequency of NKX2.5+/cTNT+ cells, or MYH6+/MYL7+ cells, or NKX2.5+/cTNT+/MYH6+/MYL7+ cells, that is or is at least 90%, 91%, 92%, 93%, 94%, 95%, 96%, 97%, or 98%. In some embodiments, the population of differentiating cardiomyocytes has a frequency of NKX2.5+/cTNT+ cells, or MYH6+/MYL7+ cells, or NKX2.5+/cTNT+/MYH6+/MYL7+ cells, that is or is at least 95%, 96%, 97%, or 98%.

In some embodiments, the population of cells may be a mixed populations of cells comprising one or more populations of cells selected from among a population of cells of any of the first aggregates, a population of dissociated cells, e.g., a population of cells dissociated from a first aggregate, a population of cells of any of the second aggregates, and/or a populations of cardiomyocytes. For instance, in some embodiments, harvesting cells at a certain time point may include harvesting a mixed populations of cells, such as a second aggregate comprising cardiomyocytes and a population of dissociated cells that did not form a second aggregate.

In some embodiments, the population of cells may be a mixed population of cells comprising one or more of (a) first aggregate, such as any of the first aggregates described herein; (b) a population of dissociated cells, such as any of the populations of dissociated cells described herein: (c) a second aggregate, such as any of the second aggregates described herein; and/or (d) a population of cardiomyocytes, e.g., a population of cardiomyocytes of the second aggregate and/or a population of cardiomyocytes differentiated from and/or dissociated from the second aggregate. In some embodiments, the population of dissociated cells was cultured in accordance with the second incubation and/or the third incubation but did not form a second aggregate.

In some embodiments, the population of cells is a mixed population of cells comprising a first aggregate and a second aggregate, such as any of the first aggregates and/or second aggregates described herein.

In some embodiments, the population of cells is a mixed population of cells comprising a first aggregate, a population of dissociated cells, and a second aggregate, such as any of the first aggregates and/or populations of dissociated cells and/or second aggregates described herein.

In some embodiments, the population of cells is a mixed population of cells comprising a first aggregate and population of dissociated cells, such as any of the first aggregates and/or populations of dissociated cells described herein.

In some embodiments, the population of cells is a mixed population of cells comprising a population of dissociated cells and a second aggregate, such as any of the populations of dissociated cells and/or second aggregates described herein.

In some embodiments, the population of cells is a population of cells differentiated from a first aggregate, such as any of the first aggregates described herein. In some embodiments, the population of cells differentiated from the first aggregate are differentiated in accordance with any of the reagents, conditions, and/or incubations described herein.

In some embodiments, the population of cells is a population of cells differentiated from a second aggregate, such as any of the second aggregates described herein. In some embodiments, the population of cells differentiated from the second aggregate are differentiated in accordance with any of the reagents, conditions, and/or incubations described herein, including any of the second and/or third incubations described herein.

In some embodiments, the population of cells is a population of cells dissociated from a first aggregate, such as any of the first aggregates described herein. In some embodiments, the dissociation of the population of cells from the first aggregate can occur by, or result from, any means and/or reagents and/or conditions.

In some embodiments, the population of dissociated cells was cultured in accordance with the second incubation and/or the third incubation but did not form a second aggregate.

In some embodiments, the first aggregate was cultured in accordance with the contacting with a dissociating agent, and/or the second incubation and/or the third incubation, but did not result in a population of dissociated cells.

In some embodiments, the population of cells is a mixed population of cells comprising one or more populations of cells selected from the group consisting of (a) a first aggregate, such as any of the first aggregates described herein, (b) a population of dissociated cells, such as any of the populations of dissociated cells described herein, (c) a second aggregate, such as any of the second aggregates described herein, (d) a population of cardiomyocytes, such as any of the populations of cardiomyocytes described herein, (e) a population of cells dissociated from a first aggregate, such as a population of cells dissociated from any of the first aggregates described herein, (f) a population of cells dissociated from a second aggregate, such as a population of cells dissociated from any of the second aggregates described herein, (g) a first aggregate, such as any of the first aggregates described herein, cultured in accordance with the conditions of the second incubation and/or third incubation, without being dissociated into a population of dissociated cells, and (h) a population of dissociated cells, such as any of the populations of dissociated cells described herein, cultured in accordance with the conditions of the second incubation and/or third incubation, without forming a second aggregate. In some embodiments, one or more of the first aggregate, the population of dissociated cells, the second aggregate, the population of cardiomyocytes, the population of cells dissociated from a first aggregate, the population of cells dissociated from a second aggregate, the first aggregate cultured in accordance with the conditions of the second incubation and/or third incubation without being dissociated into a population of dissociated cells, and the population of dissociated cells cultured in accordance with the conditions of the second incubation and/or third incubation without forming a second aggregate, were harvested on or about any one of days 3, 4, 5, 6, 7, 8, 9, 10, 11, 12, 13, 14, 15, 16, 17, 18, 19, 20, 21, 22, 23, or 24.

In some embodiments, any of such populations of cells, e.g., a population of pluripotent stem cells, a population of dissociated cells, a population of cardiomyocytes, and/or aggregates, e.g., a first aggregate and/or second aggregate, including combinations thereof, can be harvested, cryopreserved, and/or administered to a subject, such as for treatment of a disease or condition.

Cardiomyocytes disclosed herein may be differentiated from pluripotent stem cells. Methods for differentiating cardiomyocytes from pluripotent stem cells may comprise contacting an aggregate formed by culturing a population of pluripotent stem cells with a dissociating agent to form a population of dissociated cells that are then cultured under conditions to aggregate the dissociated cells and differentiate them into a population of cardiomyocytes.

Current methods to differentiate pluripotent stem cells into cardiomyocytes in suspension begin with aggregates (also sometimes referred to as clusters) of cells (e.g., having an average diameter of approximately 250 μm) that rapidly increase in size (e.g., to greater than 500 μm) during the first week of differentiation and remain at approximately that size throughout differentiation until the aggregate of cells is dissociated into single cells for harvest and cryopreservation at the end of the differentiation process.

It was believed that maintaining the aggregates at or about the size reached during the first week of differentiation (e.g., greater than 500 μm) was necessary to allow for the signaling needed to achieve differentiation of the pluripotent stem cells into mature cardiomyocytes. As such, dissociating these large aggregates during differentiation, e.g., during the first week of differentiation, and allowing them to re-aggregate into smaller aggregates than the size of the aggregate prior to such dissociation would have been unconventional.

Cardiomyocyte differentiation from pluripotent stem cells typically begins with induction using an inhibitor of glycogen synthase kinase 3 (GSK3)/activator of Wnt/β-catenin signaling, such as CHIR99021, and mesoderm formation, followed by cardiac commitment. However, reproducibility challenges may arise when differentiating pluripotent stem cells into cardiomyocytes partly due to the narrow timing range between under induction (low mesoderm) and over induction (high mesoderm followed by low cardiac commitment). Moreover, CD56+/PDFGRα+ cells in the center of large aggregates during differentiation may be prevented or delayed from differentiating into cardiomyocytes due to nutrient and/or spatial limitations that arise when the cells are present in large aggregates throughout differentiation. This can result in reduced consistency and/or reduced purity of the resulting differentiated cardiomyocyte population.

In some embodiments, other methods described below can be used which can provide for improved differentiation potential, improved consistency of cardiomyocyte differentiation, improved harvesting and cryopreservation of cardiomyocytes and precursors thereof, and the ability to cryopreserve and use dissociated aggregates for cell therapy, e.g., cardiac cell therapy.

In some embodiments, cardiomyocytes may be of differentiated from pluripotent stem cells by a method comprising: a) performing a first incubation comprising culturing a population of pluripotent stem cells under conditions to form a first aggregate, wherein the first incubation is initiated on day 0: b) contacting the first aggregate with a dissociating agent to form a population of dissociated cells: c) performing a second incubation comprising culturing the population of dissociated cells under conditions to aggregate the dissociated cells into a second aggregate; and (d) performing a third incubation comprising culturing the second aggregate under conditions to differentiate the population of cells in the second aggregate into a population of cardiomyocytes. In some embodiments, the method is performed in suspension. In some embodiments, the first incubation, the contacting, the second incubation, and the third incubation are each performed in suspension. In some embodiments, one or more of the first incubation, the contacting, the second incubation, and the third incubation are each performed in suspension.

### Pluripotent Stem Cells

In some embodiments, the population of pluripotent stem cells (PSCs) is a population of any pluripotent stem cells, e.g., any pluripotent stem cells that are capable of differentiating into cardiomyocytes. In some embodiments, the cardiomyocytes are derived from pluripotent stem cells.

In some embodiments, the pluripotent stem cells are selected from the group consisting of induced pluripotent stem cells, embryonic stem cells, bone marrow-mesenchymal stem cells, cardiac tissue stem cells, and adipose tissue stem cells. In some embodiments, the pluripotent stem cells are human pluripotent stem cells or are human-derived pluripotent stem cells. In some embodiments, the pluripotent stem cells are induced pluripotent stem cells (iPSCs). In some embodiments, the iPSCs are derived from a donor, such as a human donor.

A population of iPSCs can be generated using any available method. A variety of different methods of generating pluripotent stem cells (generally referred to as iPSCs; miPSCs for murine cells, or hiPSCs for human cells) are known. The original induction was done from mouse embryonic or adult fibroblasts using the viral introduction of four transcription factors, Oct3/4, Sox2, c-Myc and Klf4; see Takahashi and Yamanaka Cell 126:663-676 (2006), hereby incorporated by reference in its entirety and specifically for the techniques outlined therein. Since then, a number of methods have been developed: see Seki et al, World J. Stem Cells 7(1): 116-125 (2015) for a review, and Lakshmipathy and Vermuri, editors, Methods in Molecular Biology: Pluripotent Stem Cells, Methods and Protocols, Springer 2013, both of which are hereby expressly incorporated by reference in their entirety, and in particular for the methods for generating hiPSCs (see for example Chapter 3 of the latter reference).

Generally, iPSCs are generated by the transient expression of one or more “reprogramming factors” in the host cell, usually introduced using episomal vectors. Under these conditions, small amounts of the cells are induced to become iPSCs (in general, the efficiency of this step is low, as no selection markers are used). Once the cells are “reprogrammed”, and become pluripotent, they lose the episomal vector(s) and produce the factors using the endogenous genes. This loss of the episomal vector(s) results in cells that are called “zero footprint” cells. This is desirable as the fewer genetic modifications (particularly in the genome of the host cell), the better. Thus, it is preferred that the resulting hiPSCs have no permanent genetic modifications.

The number of reprogramming factors that can be used or are used can vary. Commonly, when fewer reprogramming factors are used, the efficiency of the transformation of the cells to a pluripotent state goes down, as well as the “pluripotency”, e.g. fewer reprogramming factors may result in cells that are not fully pluripotent but may only be able to differentiate into fewer cell types.

In some embodiments, a single reprogramming factor, OCT4, is used. In other embodiments, two reprogramming factors, OCT4 and KLF4, are used. In other embodiments, three reprogramming factors. OCT4, KLF4 and SOX2, are used. In other embodiments, four reprogramming factors, OCT4, KLF4, SOX2 and c-Myc, are used. In other embodiments, 5, 6 or 7 reprogramming factors can be used selected from SOKMNLT; SOX2, OCT4, (POU5F1), KLF4, MYC, NANOG, LIN28, and SV40L T antigen.

In general, these reprogramming factor genes are provided on episomal vectors such as are known in the art and commercially available. For example, ThermoFisher/Invitrogen sell a sendai virus reprogramming kit for zero footprint generation of hiPSCs, see catalog number A34546. ThermoFisher also sells EBNA-based systems as well, see catalog number A14703.

In addition, there are a number of commercially available hiPSC lines available; see, e.g., the Gibco® Episomal hiPSC line, K18945, which is a zero footprint, viral-integration-free human iPSC cell line (see also Burridge et al, 2011, supra).

In general, iPSCs are made from non-pluripotent cells such as CD34+ cord blood cells, fibroblasts, etc., by transiently expressing the reprogramming factors as described herein. For example, successful iPSCs were also generated using only Oct3/4, Sox2 and Klf4, while omitting the C-Myc, although with reduced reprogramming efficiency.

In general, iPSCs are characterized by the expression of certain factors that include KLF4, Nanog, OCT4, SOX2, ESRRB, TBX3, c-Myc and TCL1. New or increased expression of these factors may be via induction or modulation of an endogenous locus or from expression from a transgene.

For example, murine iPSCs can be generated using the methods of Diecke et al, Sci Rep. 2015, Jan. 28:5:8081 (doi: 10.1038/srep08081), hereby incorporated by reference in its entirety and specifically for the methods and reagents for the generation of the miPSCs. See also, e.g., Burridge et al., PLoS One, 2011 6(4): 18293, hereby incorporated by reference in its entirety and specifically for the methods outlined therein.

In some embodiments, PSCs (e.g., iPSCs) generated by any of the methods described herein and/or known in the art are differentiated into cardiomyocytes, such as to produce a composition highly enriched in cardiomyocytes.

The PSCs (e.g., iPSCs) can be differentiated into cardiomyocytes by any known methods, including but not limited to those described in Murry and Keller, Cell (2008) 132(4):661-80; Burridge et al., Cell Stem Cell (2012) 10:16-28; Lian et al., Nature Protocols (2013) 8:162-65; Batalov and Feiberg, Biomark. Insight (2015) 10(Suppl. 1):71-6; Denning et al., Biochim. Biophys. Acta Mol. Cell Res. (2016) 1863:1728-48; Breckwoldt et al., Nature Protocols (2017) 12:1177-97; Guo et al., Stem Cell Res. And Ther. (2018) 9:44; and Leitolis et al., Front. Cell Dev. Biol. (2019) 8:164.

In some embodiments, the cardiomyocytes are allogeneic to a subject receiving a transplant of the cardiomyocytes. Thus, in some embodiments, the PSCs (e.g. iPSCs) from which cardiomyocytes are derived are engineered to be hypoimmunogenic by any known methods.

For example, nucleic acid sequences may be modified within PSCs (e.g., iPSCs) to generate hypoimmunogenic PSCs. Technologies to modify nucleic acid sequences within cells include homologous recombination, knock-in, knock-out, ZFNs (zinc finger nucleases), TALENs (transcription activator-like effector nucleases). CRISPR (clustered regularly interspaced short palindromic repeats)/Cas9, and other site-specific nuclease technologies. These techniques enable double-strand DNA breaks at desired locus sites. These controlled double-strand breaks promote homologous recombination at the specific locus sites. This process focuses on targeting specific sequences of nucleic acid molecules, such as chromosomes, with endonucleases that recognize and bind to the sequences and induce a double-stranded break in the nucleic acid molecule. The double-strand break is repaired either by an error-prone non-homologous end-joining (NHEJ) or by homologous recombination (HR).

A number of different techniques can be used to engineer the PSCs (e.g., iPSCs) to be hypo-immunogenic, including those described in WO 2020/018615, incorporated herein by reference in its entirety. In some embodiments, engineering of the PSCs (iPSCs) to be hypoimmunogenic reduces an immune response of the recipient to the cells, including cardiomyocytes differentiated from the hypoimmunogenic PSCs (e.g., iPSCs).

In some embodiments, culturing the population of pluripotent stem cells can be performed by seeding pluripotent stem cells at any concentration suitable for forming an aggregate, e.g., an aggregate of between or between about 300 and about 700 μm in diameter by at or about day 2, 3, 4, 5, or 6.

In some embodiments, the population of pluripotent stem cells has a viable cell concentration of between or between about 1×105 and 3×107 cells/mL, 2×105 and 2×107 cells/mL, 3×105 and 1×107 cells/mL, 4×105 and 9×106 cells/mL, 5×105 and 8×106 cells/mL, 6×105 and 7×106 cells/mL, 7×105 and 6×106 cells/mL, 8×105 and 5×106 cells/mL, 9×105 and 4×106 cells/mL, 9×105 and 3×106 cells/mL, 1×106 and 2×106 cells/mL, 1.1×106 and 1.9×106 cells/mL, 1.2×106 and 1.8×106 cells/mL, 1.25×106 and 1.75×106 cells/mL. In some embodiments, the population of pluripotent stem cells has a viable cell concentration of between or between about 1×105 and 3×107 cells/mL, 2×105 and 2×107 cells/mL, 3×105 and 1×107 cells/mL, 4×105 and 9×106 cells/mL, 5×105 and 8×106 cells/mL, 6×105 and 7×106 cells/mL, 7×105 and 6×106 cells/mL, 8×105 and 5×106 cells/mL, 9×105 and 4×106 cells/mL, 9×105 and 3×106 cells/mL, 1×106 and 2×106 cells/mL, 1.1×106 and 1.9×106 cells/mL, 1.2×106 and 1.8×106 cells/mL, 1.25×106 and 1.75×106 cells/mL at or about the time of seeding on day 0 or at or about the time the first incubation is initiated on day 0.

In some embodiments, the population of pluripotent stem cells has a viable cell concentration of or of about 1.0×106 cells/mL, 1.05×106 cells/mL, 1.1×106 cells/mL, 1.15×106 cells/mL, 1.2×106 cells/mL, 1.25×106 cells/mL, 1.3×106 cells/mL, 1.35×106 cells/mL, 1.4×106 cells/mL, 1.45×106 cells/mL, 1.5×106 cells/mL, 1.55×106 cells/mL, 1.6×106 cells/mL, 1.65×106 cells/mL, 1.7×106 cells/mL, 1.75×106 cells/mL, 1.8×106 cells/mL, 1.85×106 cells/mL, 1.9×106 cells/mL, 1.95×106 cells/mL, 2.0×107 cells/mL, 2.05×107 cells/mL, 2.1×107 cells/mL, 2.15×107 cells/mL, or 2.2×107 cells/mL. In some embodiments, the population of pluripotent stem cells has a viable cell concentration of or of about 1.0×106 cells/mL, 1.05×106 cells/mL, 1.1×106 cells/mL, 1.15×106 cells/mL, 1.2×106 cells/mL, 1.25×106 cells/mL, 1.3×106 cells/mL, 1.35×106 cells/mL, 1.4×106 cells/mL, 1.45×106 cells/mL, 1.5×106 cells/mL, 1.55×106 cells/mL, 1.6×106 cells/mL, 1.65×106 cells/mL, 1.7×106 cells/mL, 1.75×106 cells/mL, 1.8×106 cells/mL, 1.85×106 cells/mL, 1.9×106 cells/mL, 1.95×106 cells/mL, 2.0×107 cells/mL, 2.05×107 cells/mL, 2.1×107 cells/mL, 2.15×107 cells/mL, or 2.2×107 cells/mL at or about the time of seeding on day 0 or at or about the time the first incubation is initiated on day 0.

In some embodiments, the population of pluripotent stem cells has a viable cell concentration of or of about 1.4×106 cells/mL, 1.45×106 cells/mL, 1.5×106 cells/mL, 1.55×106 cells/mL, 1.6×106 cells/mL, 1.65×106 cells/mL, 1.7×106 cells/mL, 1.75×106 cells/mL, or 1.8×106 cells/mL. In some embodiments, the population of pluripotent stem cells has a viable cell concentration of or of about 1.4×106 cells/mL, 1.45×106 cells/mL, 1.5×106 cells/mL, 1.55×106 cells/mL, 1.6×106 cells/mL, 1.65×106 cells/mL, 1.7×106 cells/mL, 1.75×106 cells/mL, or 1.8×106 cells/mL at or about the time of seeding on day 0 or at or about the time the first incubation is initiated on day 0.

### Reagents and Conditions for Culturing and Dissociation

In some embodiments, one or more of the reagents and conditions for differentiating cardiomyocytes from pluripotent stem cells, e.g., the first incubation, the second incubation, and/or the third incubation, can include, or be modified from, known methods.

Soluble factors important for embryonic cardiac development include Activin A, BMP4, nodal. Wnt agonists and antagonists, bFGF and other molecules (Conlon et al, Development 120(7): 1919 (1994); Lough et al, Dev. Biol. (1996) 178(1): 198: Mima et al, PNAS (1995) 92(2):467; Zaffran and Frasch, Circ. Res. (2002) 91 (6), 457). Any suitable method of inducing cardiomyocyte differentiation may be used, for example, any of those described in Fujiwara et al., PLoS One. (2001) 6(2):e16734; Dambrot et al., Biochem J. (2011) 434(1):25-35; Foldes et al., J Mol Cell Cardiol. (2011) 50(2):367-76; Wang et al., Sci China Life Sci. (2010) 53(5):581-9: Chen et al., J Cell Biochem. (2010) (1):29-39; Yang et al., Nature (2008) 453:524-28; Kattman et al., Cell Stem Cell (2011) 8:228-40; Laflamme et al., Nat. Biotechnol. (2007) 25: 1015-24: Paige et al., PLoS One (2010) 5(6): e11134: Xu et al., Regen Med (2011) 6(1):53-66; Mignone et al., Circ J (2010) 74(12):2517-26; and Takei et al., Am J Physiol Heart Circ Physiol. (2009) 296(6):H1793-803, each herein incorporated by reference in its entirety. PSCs (e.g., iPSCs or ESCs) can also be differentiated into cardiomyocytes by any of the methods described in WO2013013206 and WO2013056072, each incorporated by reference in its entirety.

In some embodiments, the method of differentiating cardiomyocytes from pluripotent stem cells comprises: a) performing a first incubation comprising culturing a population of pluripotent stem cells under conditions to form a first aggregate, wherein the first incubation is initiated on day 0; b) contacting the first aggregate with a dissociating agent to form a population of dissociated cells; c) performing a second incubation comprising culturing the population of dissociated cells under conditions to aggregate the dissociated cells into a second aggregate; and (d) performing a third incubation comprising culturing the second aggregate under conditions to differentiate the population of cells in the second aggregate into a population of cardiomyocytes. Exemplary reagents and conditions for the first incubation, dissociation via contacting the first aggregate with a dissociating agent, the second incubation, and the third incubation are provided below, which are not intended to be limiting.

### First Incubation

The first incubation is initiated on day 0 and comprises culturing a population of pluripotent stem cells under conditions to form a first aggregate.

In some embodiments, the first incubation comprises culturing the population of pluripotent stem cells under any conditions suitable for allowing the pluripotent stem cells to form a first aggregate, such as any medias, reagents, and/or conditions used on one or more of days 0, 1, 2, 3, 4, 5, and 6 in any known method for differentiating cardiomyocytes from pluripotent stem cells, such as, e.g., described in Fujiwara et al., PLoS One. (2001) 6(2):e16734: Dambrot et al., Biochem J. (2011) 434(1):25-35: Foldes et al., J Mol Cell Cardiol. (2011) 50(2):367-76; Wang et al., Sci China Life Sci. (2010) 53(5):581-9; Chen et al., J Cell Biochem. (2010) (1):29-39; Yang et al., Nature (2008) 453:524-28; Kattman et al., Cell Stem Cell (2011) 8:228-40; Laflamme et al., Nat. Biotechnol. (2007) 25: 1015-24; Paige et al., PLoS One (2010)5(6): el1134; Xu et al., Regen Med (2011)6(1):53-66: Mignone et al., Circ J (2010) 74(12):2517-26; Takei et al., Am J Physiol Heart Circ Physiol. (2009) 296(6):H1793-803; WO2013013206; and WO2013056072. In some embodiments, the first incubation comprises culturing the population of pluripotent stem cells in any one or more medias suitable for allowing the pluripotent stem cells to form a first aggregate, such as any media used on one or more of days 0, 1, 2, 3, 4, 5, and 6, that is known, e.g., as described in Fujiwara et al., PLoS One. (2001) 6(2):e16734; Dambrot et al., Biochem J. (2011) 434(1):25-35; Foldes et al., J Mol Cell Cardiol. (2011) 50(2):367-76: Wang et al., Sci China Life Sci. (2010) 53(5):581-9; Chen et al., J Cell Biochem. (2010) (1):29-39; Yang et al., Nature (2008) 453:524-28; Kattman et al., Cell Stem Cell (2011) 8:228-40; Laflamme et al., Nat. Biotechnol. (2007) 25: 1015-24; Paige et al., PLoS One (2010) 5(6): e 11134; Xu et al., Regen Med (2011) 6(1):53-66; Mignone et al., Circ J (2010) 74(12):2517-26; Takei et al., Am J Physiol Heart Circ Physiol. (2009) 296(6):H1793-803; WO2013013206; and WO2013056072.

In some embodiments, the first incubation comprises culturing the population of pluripotent stem cells in a differentiation day 0 (DD0) media comprising an inhibitor of glycogen synthase kinase 3 (GSK3)/activator of Wnt/β-catenin signaling. In some embodiments, the first incubation comprises culturing the population of pluripotent stem cells in a DD0 media comprising an inhibitor of GSK3/activator of Wnt/(3-catenin signaling, and L-alanyl-L-glutamine (a dipeptide substitute for L-glutamine). In some embodiments, the DD0 media further comprises a serum-free and insulin-free growth supplement. In some embodiments, the serum-free and insulin-free growth supplement is a B27™ minus insulin supplement (Cat #A18956-01; Life Technologies).

In some embodiments, the DD0 media comprises a base media that is MCDB 131 medium (Cat #10372-019; Life Technologies).

In some embodiments, the L-alanyl-L-glutamine is GlutaMax™, such as CTS GlutamMax™ (Cat #A12860-01; Life Technologies) or GlutaMax™ (Cat #35050-061: Life Technologies).

In some embodiments, the inhibitor of GSK-3α and/or GSK-3β is an inhibitor of GSK-3α and GSK-3β. In some embodiments, the inhibitor of GSK-3α and/or GSK-3β is CHIR99021. In some embodiments, the concentration of CHIR99021 is between about 4 μM and about 8 μM. In some embodiments, the concentration of CHIR99021 is between about 5 μM and about 7 μM. In some embodiments, the concentration of CHIR99021 is about 5, 5.5, 6, 6.5, or 7 μM, or any value in between any of the aforementioned values. In some embodiments, the concentration of CHIR99021 is about 5, 5.1, 5.2. 5.3, 5.4, 5.5, 5.6, 5.7, 5.8, 5.9, 6, 6.1, 6.2, 6.3.6.4, 6.5, 6.6, 6.7, 6.8. 6.9, or 7 μM. In some embodiments, the concentration of CHIR99021 is between about 5 μM and about 6 μM or is between about 4.5 μM and about 6.5 μM. In some embodiments, the concentration of CHIR99021 is about 5 μM. In some embodiments, the concentration of CHIR99021 is about 6 μM. In some embodiments, the concentration of CHIR99021 is about 5 μM or about 6 μM.

In some embodiments, the first incubation comprises culturing the population of pluripotent stem cells in the media comprising the inhibitor of GSK-3α and/or GSK-3β, e.g., the DD0 media, on day 0. In some embodiments, the first incubation comprises culturing the population of pluripotent stem cells in the media comprising the inhibitor of GSK-3α and/or GSK-3β, e.g., the DD0 media, on day 0 and day 1. In some embodiments, the first incubation comprises culturing the population of pluripotent stem cells in the media comprising the inhibitor of GSK-3α and/or GSK-3β, e.g., the DD0 media, on days 0 and 1, and one or more subsequent days of the first incubation.

In some embodiments, the first incubation comprises culturing the population of pluripotent stem cells in a media, e.g., a differentiation day 1 (DD1) media, comprising L-alanyl-L-glutamine and a serum-free and insulin-free growth supplement. In some embodiments, the media, e.g., DD1 media, comprises a base media that is MCDB 131 medium. In some embodiments, the L-alanyl-L-glutamine is GlutaMax™ (Cat #35050-061: Life Technologies). In some embodiments, the serum-free and insulin-free growth supplement is a B27™ minus insulin supplement (Cat #A18956-01; Life Technologies).

In some embodiments, the first incubation comprises culturing the population of pluripotent stem cells in a media, e.g., DD1 media, comprising L-alanyl-L-glutamine and a serum-free and insulin-free growth supplement on or about day 1. In some embodiments, the first incubation comprises culturing the population of pluripotent stem cells in a media, e.g., DD1 media, comprising L-alanyl-L-glutamine and a serum-free and insulin-free growth supplement on day 1 and day 2. In some embodiments, the first incubation comprises culturing the population of pluripotent stem cells in a media, e.g., DD1 media, comprising L-alanyl-L-glutamine and a serum-free and insulin-free growth supplement on day 1 and day 2, and one or more subsequent days of the first incubation.

In some embodiments, after day 0, the first incubation comprises culturing the population of pluripotent stem cells in a media, e.g., a differentiation day 2 (DD2) media, comprising an inhibitor of Wntβ-catenin signaling. In some embodiments, the media, e.g., DD2 media, comprises L-alanyl-L-glutamine and a serum-free differentiation supplement. In some embodiments, the media, e.g., DD2 media, comprises a base media that is MCDB 131 medium. In some embodiments, the inhibitor of Wnt/β-catenin signaling is selected from the group consisting of WIKI4, NSC668036, iCRT3, iCRT5, iCRT14, IWP-2, XAV-939, ICG-001, LGK-974, OMP-18R5, FJ9, IWR-1-endo, KY02111, PFK115-584, Wnt-059, DKK1, FH-535, Box5, and Peptide Pen-N3. In some embodiments, the inhibitor of Wnt/β-catenin signaling is WIKI4. In some embodiments, the L-alanyl-L-glutamine is GlutaMax™ (Cat #35050-061: Life Technologies). In some embodiments, the serum-free differentiation supplement is a B27™ supplement (Cat #A1486701: Life Technologies).

In some embodiments, the culturing the population of pluripotent stem cells in the media, e.g., DD2 media, comprising the inhibitor of Wnt/β-catenin signaling begins approximately 36 to 48 hours after the first incubation is initiated on day 0. In some embodiments, the culturing the population of pluripotent stem cells in the media, e.g., DD2 media, comprising the inhibitor of Wntβ-catenin signaling begins approximately 40 hours after the first incubation is initiated on day 0.

In some embodiments, the first incubation comprises culturing the population of pluripotent stem cells in a media, e.g., DD2 media, comprising an inhibitor of Wnt/β-catenin signaling on or about day 2. In some embodiments, the first incubation comprises culturing the population of pluripotent stem cells in a media, e.g., DD2 media, comprising an inhibitor of Wnt/β-catenin signaling on or about one or more of day 1, day 2, and day 3. In some embodiments, the first incubation comprises culturing the population of pluripotent stem cells in a media, e.g., DD2 media, comprising an inhibitor of Wntβ-catenin signaling on or about day 2 and day 3. In some embodiments, the first incubation comprises culturing the population of pluripotent stem cells in a media, e.g., DD2 media, comprising an inhibitor of Wnt/β-catenin signaling on or about day 2 and day 3, and one or more subsequent days of the first incubation.

In some embodiments, the first incubation comprises culturing the population of pluripotent stem cells in a media, e.g., a differentiation day 3 (DD3) media, comprising L-alanyl-L-glutamine and a serum-free differentiation supplement. In some embodiments, the media, e.g., DD3 media, comprises a base media that is MCDB 131 medium. In some embodiments, the L-alanyl-L-glutamine is GlutaMax™ (Cat #35050-061: Life Technologies). In some embodiments, the serum-free differentiation supplement is a B27™ supplement (Cat #A1486701; Life Technologies).

In some embodiments, the first incubation comprises culturing the population of pluripotent stem cells in a media, e.g., a differentiation day 3 (DD3) media, comprising L-alanyl-L-glutamine and a serum-free differentiation supplement on or about day 3. In some embodiments, the first incubation comprises culturing the population of pluripotent stem cells in a media, e.g., a differentiation day 3 (DD3) media, comprising L-alanyl-L-glutamine and a serum-free differentiation supplement on or about one or more of about day 3, day 4, day 5, and day 6. In some embodiments, the first incubation comprises culturing the population of pluripotent stem cells in a media, e.g., a differentiation day 3 (DD3) media, comprising L-alanyl-L-glutamine and a serum-free differentiation supplement on or about day 3 and day 4. In some embodiments, the first incubation comprises culturing the population of pluripotent stem cells in a media, e.g., a differentiation day 3 (DD3) media, comprising L-alanyl-L-glutamine and a serum-free differentiation supplement on or about day 3 and day 4, and one or more subsequent days of the first incubation.

In some embodiments, the first aggregate that is formed during the first incubation is between or between about 300 and about 1,000 μm in diameter. In some embodiments, the first aggregate that is formed during the first incubation is between or between about 300 and about 500, 600, 700, 800. 900, or 1.000 μm in diameter. In some embodiments, the first aggregate that is formed during the first incubation is between or between about 300 and about 1,000 μm in diameter on or about day 2, day 3, day 4, day 5, or day 6. In some embodiments, the first aggregate that is formed during the first incubation is between or between about 300 and about 500, 600, 700. 800, 900, or 1,000 μm in diameter on or about day 2, day 3, day 4, day 5, or day 6. In some embodiments, the first aggregate that is formed during the first incubation is between or between about 300 and about 700 μm in diameter. In some embodiments, the first aggregate that is formed during the first incubation is between or between about 300 and about 700 μm in diameter on or about day 2, day 3, day 4, day 5, or day 6. In some embodiments, the first aggregate that is formed during the first incubation is between or between about 350 and 650 μm, 400 and 650 μm, 450 and 650 μm, 450 and 600 μm, 500 and 650 μm, 550 and 650 μm, or 550 and 600 μm in diameter on or about day 2, day 3, day 4, day 5, or day 6. In some embodiments, the first aggregate that is formed during the first incubation is between or between about 500 and about 650 μm in diameter. In some embodiments, the first aggregate that is formed during the first incubation is between or between about 550 and about 600 μm in diameter. In some embodiments, the first aggregate that is formed during the first incubation is between or between about 300 and about 700 μm in diameter on or about day 3 or day 4. In some embodiments, the first aggregate that is formed during the first incubation is between or between about 350 and 650 μm, 400 and 650 μm, 450 and 650 μm, 450 and 600 μm, 500 and 650 μm, 550 and 650 μm, or 550 and 600 μm in diameter on or about day 3 or day 4. In some embodiments, the first aggregate that is formed during the first incubation is between or between about 500 and about 650 μm in diameter on or about day 3 or day 4. In some embodiments, the first aggregate that is formed during the first incubation is between or between about 550 and about 600 μm in diameter on or about day 3 or day 4.

In some embodiments, the first incubation is initiated when the population of pluripotent stem cells is first contacted by the DD0 media.

In some embodiments, the first incubation occurs in suspension. In some embodiments, the first incubation comprises culturing the population of pluripotent stem cells in suspension. In some embodiments, the first incubation comprises culturing the population of pluripotent stem cells in suspension for one or more days, two or more days, three or more days, or four or more days, or five or more days of the first incubation. In some embodiments, the first incubation comprises culturing the population of pluripotent stem cells in suspension for the entirety of the first incubation.

### Dissociation Using a Dissociating Agent

The first aggregate that is formed during the first incubation is dissociated using a dissociating agent to form a population of dissociated cells. For instance, in some embodiments, the method comprises contacting the first aggregate with a dissociating agent to form a population of dissociated cells.

In some embodiments, the contacting occurs when the first aggregate is between or between about 300 and 1,000 μm in diameter. In some embodiments, the contacting occurs when the first aggregate is between or between about 300 and 500.600, 700, 800, 900, or 1,000 μm in diameter.

In some embodiments, the contacting the first aggregate with the dissociating agent occurs when the first aggregate is between or between about 300 and about 1,000 μm in diameter, such as between or between about 300 and 500, 600, 700, 800, 900, or 1,000 μm in diameter, or between or between about 350 and 650 μm, 400 and 650 μm, 450 and 650 μm, 450 and 600 μm, 500 and 650 μm, 550 and 650 μm, or 550 and 600 μm in diameter. In some embodiments, the first aggregate is between or between about 500 and about 650 μm in diameter. In some embodiments, the first aggregate is between or between about 550 and about 600 μm in diameter. In some embodiments, the contacting the first aggregate with the dissociating agent occurs when the first aggregate is between or between about 400 and 1,000, 400 and 950, 400 and 900, 400 and 850, 400 and 800, 400 and 750, 400 and 700, 450 and 1,000, 450 and 950, 450 and 900, 450 and 850, 450 and 800, 450 and 750, or 450 and 700 μm in diameter.

In some embodiments, the contacting the first aggregate with the dissociating agent occurs at a time when the first aggregate is between or between about 300 and about 1,000 μm in diameter. In some embodiments, the contacting the first aggregate with the dissociating agent occurs at a time when the first aggregate is between or between about 300 and about 500, 600, 700, 800, 900, or 1,000 μm in diameter. In some embodiments, the contacting the first aggregate with the dissociating agent occurs at a time when the first aggregate is between or between about 350 and 650 μm, 400 and 650 μm, 450 and 650 μm, 450 and 600 μm, 500 and 650 μm, 550 and 650 μm, or 550 and 600 μm in diameter. In some embodiments, the contacting the first aggregate with the dissociating agent occurs at a time when the first aggregate is between or between about 400 and 1,000, 400 and 950, 400 and 900. 400 and 850, 400 and 800, 400 and 750, 400 and 700, 450 and 1,000, 450 and 950. 450 and 900, 450 and 850. 450 and 800, 450 and 750, or 450 and 700 μm in diameter. In some embodiments, the contacting the first aggregate with the dissociating agent occurs at a time when the first aggregate is between or between about 500 and about 650 μm in diameter. In some embodiments, the contacting the first aggregate with a dissociating agent occurs at a time when the first aggregate is between or between about 550 and about 600 μm in diameter. In some embodiments, the time, e.g., the time when the first aggregate is contacted with the dissociating agent, is on or about day 2, day 3, day 4, day 5, or day 6. In some embodiments, the time, e.g., the time when the first aggregate is contacted with the dissociating agent, is on or about day 2 or day 3. In some embodiments, the time, e.g., the time when the first aggregate is contacted with the dissociating agent, is on or about day 3 or day 4. In some embodiments, the time, e.g., the time when the first aggregate is contacted with the dissociating agent, is on or about day 4 or day 5. In some embodiments, the time, e.g., the time when the first aggregate is contacted with the dissociating agent, is on or about day 4.

In some embodiments, the contacting occurs on or about any one of days 2, 3, 4, 5, and 6. In some embodiments, the contacting occurs on or about day 2 or day 3. In some embodiments, the contacting occurs on or about day 3 or day 4. In some embodiments, the contacting occurs on or about day 4 or day 5. In some embodiments, the contacting occurs on or about day 5 or day 6. In some embodiments, the contacting occurs on or about day 4. In some embodiments, the contacting occurs on or about any one of days 2 to 6, or on or about any one of days 3 to 6, or on any one of days 4 to 6.

In some embodiments, the contacting occurs on the last day of the first incubation, following the first incubation. In some embodiments, the contacting occurs on the day following the last day of the first incubation.

In some embodiments, at the time of the contacting, the first aggregate comprises a frequency of CD56+/PDGFRα+ cells that is or is at least 70%, 71%, 72%, 73%, 74%, 75%, 76%, 77%, 78%, 79% 80%, 81%, 82%, 83%, 84%, 85%, 86%, 87%, 88%, 89%, 90%, 91%, 92%, 93%, 94%, or 95%. In some embodiments, at the time of the contacting, the first aggregate comprises a frequency of CD56+/PDGFRα+ cells that is or is at least 80%, 81%, 82%, 83%, 84%, 85%, 86%, 87%, 88%, 89%, 90%, 91%, 92%, 93%, 94%, or 95%. In some embodiments, at the time of the contacting, the first aggregate comprises a frequency of CD56+/PDGFRα+ cells that is or is about 80%, 81%, 92%, 83%, 84%, 85%, 86%, 87%, 88%, 89%, 90%, 91%, 92%, 93%, 94%, 95%, 96%, 97%, 98%, or 99%.

The dissociating agent can, in some embodiments, be or comprise any agent that allows for an aggregate of cells to dissociate from one another, without affecting the ability of the dissociated cells to subsequently proliferate and/or grow and/or differentiate under suitable culture conditions. In some embodiments, the dissociating agent is or comprises a cleavage enzyme. In some embodiments, the cleavage enzyme is a protease. In some embodiments, the protease is a recombinant enzyme that cleaves a peptide bond on the C-terminal side of a lysine or arginine residue. In some embodiments, the protease is TrypLE™ Select (Cat #A1217702; Life Technologies). In some embodiments, the protease is a recombinant enzyme that cleaves a peptide bond on the C-terminal side of a lysine or arginine residue, such as TrypLE™ Select. In some embodiments, the protease is an endopeptidase. In some embodiments, the endopeptidase is trypsin. In some embodiments, the protease is selected from the group consisting of trypsin, collagenase, chymotrypsin, elastase, hyaluronidase, papin, and dispase. In some embodiments, the collagenase is collagenase type I, collagenase type II, or collagenase type III. In some embodiments, the collagenase is collagenase type I, collagenase type II, or collagenase type III. In some embodiments, the protease is collagenase. In some embodiments, the protease is hyaluronidase.

In some embodiments, the contacting occurs for a duration that is sufficient to result in a population of dissociated cells. In some embodiments, the contacting occurs for a duration of about 15 minutes to about 2 hours. In some embodiments, the contacting occurs for a duration of about 30 minutes to about 90 minutes, about 30 minutes to about 75 minutes, about 40 minutes to about 60 minutes, about 40 minutes to about 55 minutes, about 40 minutes to about 50 minutes, about 45 minutes to 55 minutes, or about 45 minutes to about 50 minutes. In some embodiments, the contacting occurs for a duration of about 40 to about 55 minutes. In some embodiments, the contacting occurs for a duration of about 45 to about 50 minutes.

In some embodiments, the method comprises, during the contacting, agitating the first aggregate. In some embodiments, the agitating is performed using a shaker, e.g., a platform shaker. In some embodiments, the agitating is performed at a revolutions per minute (RPM) of between or between about 20 and 100 RPM. In some embodiments, the agitating is performed at an RPM of between about 20 and 100, 20 and 90, 20 and 80, 20 and 70, 20 and 60, 20 and 50, 30 and 100, 30 and 90, 30 and 80, 30 and 70, 30 and 60, 30 and 50, 40 and 100, 40 and 90, 40 and 80, 40 and 70, 40 and 60, 40 and 50 RPM, 50 and 100. 50 and 90. 50 and 80, 50 and 70, 50 and 60, 60 and 100, 60 and 90, 60 and 80, or 60 and 70 RPM. In some embodiments, the agitating is performed at an RPM of between or between about 45 and 55 RPM, such as at or about 50 RPM. In some embodiments, the agitating is performed at or about 50 RPM. In some embodiments, the agitating is performed at any one or more of the aforementioned RPMs and/or ranges of RPM, such as one, two, three, or four or more different RPMs, each for a duration of time.

In some embodiments, the method further comprises, during and/or after the contacting, triturating the population of dissociated cells. In some embodiments, the triturating is performed by passing the population of dissociated cells through a pipette one or more times. In some embodiments, the triturating is performed by passing the population of dissociated cells through a pipette at or at least 5, 6, 7, 8, 9, 10, 11, 12, 13, 14, 15, 16, 17, 18, 19, 20, 21, 22, 23, 24, or 25 times. In some embodiments, the triturating is performed by passing the population of dissociated cells through a pipette about 15 to about 25 times, such as about 20 times. In some embodiments, the triturating results in a population of dissociated cells that has a higher frequency of single cells than if the method did not comprise triturating the population of dissociated cells.

In some embodiments, the population of dissociated cells has a viable cell density of between or between about 1×105 and 3×107 cells/mL, 2×105 and 2×107 cells/mL, 3×105 and 1×107 cells/mL, 4×105 and 9×106 cells/mL, 5×105 and 8×106 cells/mL, 6×105 and 7×106 cells/mL, 7×105 and 6×106 cells/mL, 8×105 and 5×106 cells/mL, 9×105 and 4×106 cells/mL, 9×105 and 3×106 cells/mL, 1×106 and 2×106 cells/mL, 1.1×106 and 1.9×106 cells/mL, 1.2×106 and 1.8×106 cells/mL, 1.25×106 and 1.75×106 cells/mL.

In some embodiments, after the contacting and/or triturating, the population of dissociated cells has a viable cell density of between or between about 1×105 and 3×107 cells/mL, 2×105 and 2×107 cells/mL, 3×105 and 1×107 cells/mL, 4×105 and 9×106 cells/mL, 5×105 and 8×106 cells/mL, 6×105 and 7×106 cells/mL, 7×105 and 6 ×106 cells/mL, 8×105 and 5×106 cells/mL, 9×105 and 4×106 cells/mL, 9×105 and 3×106 cells/mL, 1×106 and 2×106 cells/mL, 1.1×106 and 1.9×106 cells/mL, 1.2×106 and 1.8 ×106 cells/mL, 1.25×106 and 1.75×106 cells/mL.

In some embodiments, at or about one or two hours after the contacting is initiated, the population of dissociated cells has a viable cell density of between or between about 1×105 and 3×107 cells/mL, 2×105 and 2×107 cells/mL, 3×105 and 1×107 cells/mL, 4×105 and 9×106 cells/mL, 5×105 and 8×106 cells/mL, 6×105 and 7×106 cells/mL, 7 ×105 and 6×106 cells/mL, 8×105 and 5×106 cells/mL, 9×105 and 4×106 cells/mL, 9×105 and 3×106 cells/mL, 1×106 and 2×106 cells/mL, 1.1×106 and 1.9×106 cells/mL, 1.2 ×106 and 1.8×106 cells/mL, 1.25×106 and 1.75×106 cells/mL.

In some embodiments, the contacting occurs in suspension. In some embodiments, the contacting comprises contacting the first aggregate with the dissociating agent in suspension. In some embodiments, the triturating and/or agitating is performed in suspension.

### Second Incubation

The second incubation is performed following the contacting and comprises culturing the population of dissociated cells under conditions to aggregate the dissociated cells into a second aggregate.

In some embodiments, the second incubation occurs for a duration of time that is sufficient to allow the population of dissociated cells to aggregate into a second aggregate. In some embodiments, the duration of time that is sufficient to allow the population of dissociated cells to aggregate into a second aggregate is or is about one to three days. In some embodiments, the duration of time that is sufficient to allow the population of dissociated cells to aggregate into a second aggregate is or is about one day or two days. In some embodiments, the duration of time that is sufficient to allow the population of dissociated cells to aggregate into a second aggregate is or is about one day.

In some embodiments, the second incubation comprises culturing the population of dissociated cells in a media, e.g., a differentiation day 4 (DD4) media, comprising L-alanyl-L-glutamine and a serum-free differentiation supplement. In some embodiments, the media, e.g., DD3 media, comprises a base media that is MCDB 131 medium. In some embodiments, the L-alanyl-L-glutamine is GlutaMax™, such as CTS GlutamMax™ (Cat #A12860-01; Life Technologies). In some embodiments, the serum-free differentiation supplement is a B27™ supplement (Cat #A1486701; Life Technologies).

In some embodiments, the second incubation comprises culturing the population of dissociated cells, e.g., in DD4 media, on one day or two consecutive days from among days 2-7. In some embodiments, the second incubation comprises culturing the population of dissociated cells, e.g., in DD4 media, on or about day 2, 3, 4, 5, 6, or 7; and/or the second incubation comprises culturing the population of dissociated cells, e.g., in DD4 media, on or about days 2 and 3, on or about days 3 and 4, on or about days 4 and 5, on or about days 5 and 6, or on or about days 6 and 7. In some embodiments, the second incubation comprises culturing the population of dissociated cells, e.g., in DD4 media, on two consecutive days selected from among days 2 and 3, days 3 and 4, days 4 and 5, days 5 and 6, or days 6 and 7. In some embodiments, the second incubation comprises culturing the population of dissociated cells, e.g., in DD4 media, on or about day 4 and day 5. 50M

In some embodiments, the second aggregate is smaller in diameter than the first aggregate. In some embodiments, the second aggregate has a diameter that is at least 50%, 55%, 60%, 65%, 70%, 75%, 80%, 85%, 90%, or 95% smaller than the diameter of first aggregate. In some embodiments, the second aggregate, at or about one day or two days after the contacting, is smaller in diameter than the first aggregate immediately prior to the contacting. In some embodiments, the second aggregate, at or about one day or two days after the contacting, has a diameter that is at least 50%, 55%, 60%, 65%, 70%, 75%, 80%, 85%, 90%, or 95% smaller than the diameter of first aggregate immediately prior to the contacting.

In some embodiments, the diameter of aggregates, e.g., the first aggregate and/or the second aggregate, is based on the average diameter of a plurality of such aggregates, e.g., a plurality of first aggregates or a plurality of second aggregates, cultured under the same conditions.

In some embodiments, on the second day of the second incubation and/or at or about 24 hours after the contacting, e.g., at or about 24 hours after the contacting is initiated, the second aggregate that is formed is between or between about 25 and 200 μm in diameter. In some embodiments, on the second day of the second incubation and/or at or about 24 hours after the contacting, e.g., at or about 24 hours after the contacting is initiated, the second aggregate that is formed is between or between about 25 and 200, 25 and 175, 25 and 150, 25 and 125, 30 and 200, 30 and 175, 30 and 150, 30 and 125, 35 and 200, 35 and 175, 35 and 150, 35 and 125, 40 and 200, 40 and 175, 40 and 150, 40 and 125, 45 and 200, 45 and 175, 45 and 150, 45 and 125, 50 and 200, 50 and 175, 50 and 150, 50 and 125, 55 and 200, 55 and 175, 55 and 150, 55 and 125, 60 and 200, 60 and 175, 60 and 150, 60 and 125, 65 and 200, 65 and 175, 65 and 150, 65 and 125, 70 and 200, 70 and 175, 70 and 150, 70 and 125, 75 and 200, 75 and 175, 75 and 150, or 75 and 125 μm in diameter.

In some embodiments, on the second day of the second incubation and/or at or about 24 hours after contacting, e.g., at or about 24 hours after the contacting is initiated, the second aggregate that is formed: (a) has a diameter that is less than 50% of the diameter of the first aggregate immediately prior to being contacted with the dissociating agent; and/or (b) has a diameter that is between or between about 5-50% of the diameter of the first aggregate immediately prior to being contacted with the dissociating agent; and/or (c) has a diameter that is at or about 5%, 10%, 15%, 20%, 25%, 30%, 35%, 40%, 45%, or 50% of the diameter of the first aggregate immediately prior to being contacted with the dissociating agent; and/or (d) has a diameter that is between or between about 5-25% of the diameter of the first aggregate immediately prior to being contacted with the dissociating agent.

In some embodiments, on the second day of the second incubation and/or at or about 24 hours after the contacting, e.g., at or about 24 hours after the contacting is initiated, the second aggregate has a diameter that is between or between about 5-25% of the diameter of the first aggregate immediately prior to being contacted with the dissociating agent.

In some embodiments, on the second day of the second incubation and/or on the day following the day of the contacting with the dissociating agent, the second aggregate has a diameter that is between or between about 25 and 200, 25 and 175, 25 and 150, 25 and 125, 30 and 200, 30 and 175, 30 and 150, 30 and 125, 35 and 200, 35 and 175, 35 and 150, 35 and 125, 40 and 200, 40 and 175, 40 and 150, 40 and 125, 45 and 200, 45 and 175, 45 and 150, 45 and 125, 50 and 200, 50 and 175, 50 and 150, 50 and 125, 55 and 200, 55 and 175, 55 and 150, 55 and 125, 60 and 200, 60 and 175, 60 and 150, 60 and 125.65 and 200, 65 and 175, 65 and 150, 65 and 125, 70 and 200, 70 and 175, 70 and 150, 70 and 125, 75 and 200, 75 and 175, 75 and 150, or 75 and 125 μm. In some embodiments, the day following the day of the contacting with the dissociating agent is day 3, 4, 5, 6, or 7. In some embodiments, the day following the day of the contacting with the dissociating agent is day 5.

In some embodiments, on the second day of the second incubation, the second aggregate has a diameter that is between or between about 25 and 200, 25 and 175, 25 and 150, 25 and 125, 30 and 200, 30 and 175, 30 and 150, 30 and 125, 35 and 200, 35 and 175, 35 and 150, 35 and 125, 40 and 200, 40 and 175, 40 and 150, 40 and 125, 45 and 200, 45 and 175, 45 and 150, 45 and 125, 50 and 200, 50 and 175, 50 and 150, 50 and 125. 55 and 200, 55 and 175, 55 and 150, 55 and 125, 60 and 200, 60 and 175, 60 and 150, 60 and 125, 65 and 200, 65 and 175, 65 and 150, 65 and 125, 70 and 200, 70 and 175, 70 and 150, 70 and 125, 75 and 200, 75 and 175, 75 and 150, or 75 and 125 μm. In some embodiments, the second day of the second incubation is day 3, 4, 5, 6, or 7. In some embodiments, the second day of the second incubation is day 5.

In some embodiments, on the second day following the day of the contacting with the dissociating agent and/or the second day following the start of the second incubation and/or on the first day of the third incubation, the second aggregate has a diameter that is between or between about 25 and 300, 25 and 275. 25 and 250. 40 and 300, 40 and 275. 40 and 250, 50 and 300, 50 and 275, 50 and 250, 60 and 300, 60 and 275, 60 and 250, 70 and 300, 70 and 275, 70 and 250, 80 and 300, 80 and 275, or 80 and 250 μm. In some embodiments, the second day following the day of the contacting with the dissociating agent and/or the second day following the initiation of the second incubation and/or on the first day of the third incubation is or is about day 4, 5, 6, 7, or 8. In some embodiments, the second day following the day of the contacting with the dissociating agent and/or the second day following the initiation of the second incubation and/or on the first day of the third incubation is or is about day 6.

In some embodiments, the second aggregate has a diameter that is between or between about 25 and 200, 25 and 175, 25 and 150, 25 and 125, 30 and 200, 30 and 175, 30 and 150, 30 and 125, 35 and 200, 35 and 175, 35 and 150, 35 and 125, 40 and 200, 40 and 175, 40 and 150, 40 and 125, 45 and 200, 45 and 175, 45 and 150, 45 and 125, 50 and 200. 50 and 175, 50 and 150, 50 and 125, 55 and 200, 55 and 175, 55 and 150, 55 and 125, 60 and 200, 60 and 175, 60 and 150, 60 and 125, 65 and 200, 65 and 175, 65 and 150, 65 and 125, 70 and 200, 70 and 175, 70 and 150, 70 and 125, 75 and 200, 75 and 175, 75 and 150, or 75 and 125 μm, on the second day of the second incubation and/or on the first day of the third incubation. In some embodiments, the second aggregate has a diameter that is between or between about 25 and 200, 25 and 175, 25 and 150, 25 and 125, 30 and 200, 30 and 175, 30 and 150, 30 and 125, 35 and 200, 35 and 175, 35 and 150, 35 and 125, 40 and 200, 40 and 175, 40 and 150, 40 and 125, 45 and 200, 45 and 175, 45 and 150, 45 and 125, 50 and 200, 50 and 175, 50 and 150, 50 and 125, 55 and 200, 55 and 175, 55 and 150, 55 and 125, 60 and 200, 60 and 175, 60 and 150, 60 and 125, 65 and 200, 65 and 175, 65 and 150, 65 and 125, 70 and 200, 70 and 175, 70 and 150, 70 and 125, 75 and 200, 75 and 175, 75 and 150, or 75 and 125 μm, on or about day 5.

In some embodiments, the second aggregate has a diameter that is between or between about 25 and 300, 25 and 275, 25 and 250, 40 and 300, 40 and 275, 40 and 250, 50 and 300, 50 and 275, 50 and 250, 60 and 300, 60 and 275, 60 and 250, 70 and 300, 70 and 275, 70 and 250, 80 and 300, 80 and 275, or 80 and 250 μm on the second day following the initiation of the second incubation and/or on the first day of the third incubation, the second aggregate has a diameter that is between or between about 25 and 300, 25 and 275, 25 and 250, 40 and 300, 40 and 275, 40 and 250, 50 and 300, 50 and 275, 50 and 250, 60 and 300, 60 and 275, 60 and 250, 70 and 300, 70 and 275, 70 and 250, 80 and 300, 80 and 275, or 80 and 250 μm on day 6.

In some embodiments, the second aggregate has a diameter that is between or between about 25 and 400, 25 and 350, 25 and 300, 25 and 275, 25 and 250, 40 and 400, 40 and 350, 40 and 300, 40 and 275, 40 and 250, 50 and 400, 60 and 350, 50 and 300, 50 and 275, 50 and 250, 60 and 400, 60 and 350, 60 and 300, 60 and 275, 60 and 250, 70 and 400, 70 and 350, 70 and 300, 70 and 275, 70 and 250, 80 and 400, 80 and 350, 80 and 300, 80 and 275, or 80 and 250 μm on the third day following the initiation of the second incubation and/or on the second day of the third incubation. In some embodiments, the second aggregate has a diameter that is between or between about 25 and 400, 25 and 350, 25 and 300, 25 and 275, 25 and 250, 40 and 400, 40 and 350, 40 and 300, 40 and 275, 40 and 250, 50 and 400, 60 and 350, 50 and 300, 50 and 275, 50 and 250, 60 and 400, 60 and 350, 60 and 300, 60 and 275, 60 and 250, 70 and 400, 70 and 350, 70 and 300, 70 and 275, 70 and 250, 80 and 400, 80 and 350, 80 and 300, 80 and 275, or 80 and 250 μm on day 7.

In some embodiments, the second aggregate has a diameter that is between or between about 25 and 400, 25 and 350, 25 and 300, 25 and 275, 25 and 250, 40 and 400, 40 and 350, 40 and 300, 40 and 275, 40 and 250, 50 and 400, 60 and 350, 50 and 300, 50 and 275, 50 and 250, 60 and 400, 60 and 350, 60 and 300, 60 and 275, 60 and 250, 70 and 400, 70 and 350, 70 and 300, 70 and 275, 70 and 250, 80 and 400, 80 and 350, 80 and 300. 80 and 275, or 80 and 250 μm on the fourth day following the initiation of the second incubation and/or on the third day of the third incubation. In some embodiments, the second aggregate has a diameter that is between or between about 25 and 400, 25 and 350, 25 and 300, 25 and 275, 25 and 250, 40 and 400, 40 and 350, 40 and 300, 40 and 275, 40 and 250, 50 and 400, 60 and 350, 50 and 300, 50 and 275, 50 and 250, 60 and 400, 60 and 350, 60 and 300, 60 and 275, 60 and 250, 70 and 400, 70 and 350, 70 and 300, 70 and 275, 70 and 250, 80 and 400, 80 and 350, 80 and 300, 80 and 275, or 80 and 250 μm on day 8.

In some embodiments, the second incubation occurs in suspension. In some embodiments, the second incubation comprises culturing the population of dissociated cells in suspension. In some embodiments, the second incubation comprises culturing the population of dissociated cells in suspension for one or more days, such as one day, two days, or three days, of the second incubation. In some embodiments, the second incubation comprises culturing the population of dissociated cells in suspension for the entirety of the second incubation. In some embodiments, the second incubation comprises culturing the population of dissociated cells in suspension for or for about 12 to 72 hours, 12 to 60 hours, 12 to 48 hours, 12 to 36 hours, 12 to 24 hours, 18 to 72 hours, 18 to 60 hours, 18 to 48 hours, 18 to 36 hours, 18 to 24 hours, 24 to 72 hours, 24 to 60 hours, 24 to 48 hours, or 24 to 36 hours.

### Third Incubation

The third incubation is performed following the formation of the second aggregate, e.g., following the second incubation, and comprises culturing the second aggregate under conditions to differentiate the population of cells in the second aggregate into a population of cardiomyocytes.

In some embodiments, the culturing of the third incubation comprises culturing the second aggregate under any conditions suitable for differentiating the population of cells in the second aggregate into a population of cardiomyocytes. For instance, in some embodiments, the culturing of the third incubation can comprise medias, reagents, and/or conditions as described in, e.g., Fujiwara et al., PLoS One. (2001) 6(2):e16734; Dambrot et al., Biochem J. (2011) 434(1):25-35; Foldes et al., J Mol Cell Cardiol. (2011)50(2):367-76; Wang et al., Sci China Life Sci. (2010) 53(5):581-9; Chen et al., J Cell Biochem. (2010) (1):29-39; Yang et al., Nature (2008) 453:524-28; Kattman et al., Cell Stem Cell (2011) 8:228-40; Laflamme et al., Nat. Biotechnol. (2007) 25: 1015-24; Paige et al., PLoS One (2010) 5(6): e11134; Xu et al., Regen Med (2011) 6(1):53-66; Mignone et al., Circ J (2010) 74(12):2517-26; Takei et al., Am J Physiol Heart Circ Physiol. (2009) 296(6):H1793-803; WO2013013206; and WO2013056072, including those as used on any one or more of days 3 through harvesting of cardiomyocytes, e.g., at day 22, following initiation of the differentiation method on day 0.

In some embodiments, the third incubation comprises culturing the second aggregate in a media, e.g., differentiation day 6 (DD6) media, comprising glucose, and in a media, e.g., differentiation day 10 (DD10) media, comprising sodium lactate. In some embodiments, the cells are cultured in media comprising glucose and in media comprising sodium lactate non-concurrently.

In some embodiments, the third incubation comprises culturing the second aggregate in a media, e.g., differentiation day 6 (DD6) media, comprising glucose further comprises a serum-free differentiation supplement. In some embodiments, the media, e.g., DD6 media, comprises a base media that is RPMI 1640 medium (Cat #11875-093: Life Technologies). In some embodiments, the serum-free differentiation supplement is a B27™ supplement (Cat #A1486701; Life Technologies).

In some embodiments, the third incubation comprises culturing the second aggregate in the media, e.g., DD6 media, comprising glucose and the serum-free differentiation supplement for one or more days selected from among days 4 to 22 or until the population of cardiomyocytes are harvested. In some embodiments, the third incubation comprises culturing the second aggregate in the media, e.g., DD6 media, comprising glucose and the serum-free differentiation supplement on 3 or more, 4 or more, 5 or more, or 6 or more consecutive days beginning on or about day 4, day 5, day 6, or day 7. In some embodiments, the third incubation comprises culturing the second aggregate in the media, e.g., DD6 media, comprising glucose and the serum-free differentiation supplement on 3, 4, or 5 consecutive days beginning on or about day 5, 6, or 7. In some embodiments, the third incubation comprises culturing the second aggregate in the media, e.g., DD6 media, comprising glucose and the serum-free differentiation supplement on 5 consecutive days beginning on or about day 6.

In some embodiments, the media comprising sodium lactate lacks glucose. In some embodiments, the sodium lactate is sodium-S-lactate (Cat #106522: Millipore-Sigma).

In some embodiments, the third incubation comprises culturing the second aggregate in a media, e.g., DD6 media, comprising glucose beginning at the initiation of the third incubation and continuing until harvest, e.g., on or about day 22, except for a period of one, two, three, or four consecutive days beginning on or about day 7, 8, 9, 10, 11, 12, or 13, where the media, e.g., DD6 media, comprising glucose is replaced with a media, e.g., DD10 media, comprising sodium lactate. In some embodiments, the third incubation comprises culturing the second aggregate in a media, e.g., DD6 media, comprising glucose and a serum-free differentiation supplement beginning at the initiation of the third incubation and continuing until harvest, e.g., on or about day 22, except for a period of one, two, three, or four consecutive days beginning on or about day 7, 8, 9, 10, 11, 12, or 13, where the media, e.g., DD6 media, comprising glucose and the serum-free differentiation supplement is replaced with a media. e.g., DD10 media, comprising sodium lactate.

In some embodiments, the third incubation comprises culturing the second aggregate in a media, e.g., DD6 media, comprising glucose beginning at the initiation of the third incubation and continuing until harvest, e.g., on or about day 22, except for a period of three consecutive days beginning on or about day 10, e.g., on days 10, 11, and 12, where the media, e.g., DD6 media, comprising glucose is replaced with a media, e.g., DD10 media, comprising sodium lactate. In some embodiments, the third incubation comprises culturing the second aggregate in a media, e.g., DD6 media, comprising glucose and a serum-free differentiation supplement beginning at the initiation of the third incubation and continuing until harvest, e.g., on or about day 22, except for a period of three consecutive days beginning on or about day 10, e.g., on days 10, 11, and 12, where the media, e.g., DD6 media, comprising glucose and the serum-free differentiation supplement is replaced with a media, e.g., DD10 media, comprising sodium lactate.

In some embodiments, the third incubation comprises culturing the second aggregate in a media, e.g., DD10 media, comprising glucose and a serum-free differentiation supplement on one or more days selected from among days 7 to 15. In some embodiments, the third incubation comprises culturing the second aggregate in a media, e.g., DD10 media, comprising glucose and a serum-free differentiation supplement on 1, 2 or more, 3 or more, or 4 or more consecutive days selected from among days 7 to 15. In some embodiments, the third incubation comprises culturing the second aggregate in a media, e.g., DD10 media, comprising glucose and a serum-free differentiation supplement on 1, 2, 3, or 4 consecutive days beginning on or about day 7, 8, 9, 10, 11, 12, or 13. In some embodiments, the third incubation comprises culturing the second aggregate in a media, e.g., DD10 media, comprising glucose and a serum-free differentiation supplement on 3 consecutive days beginning on or about day 9, 10, or 11. In some embodiments, the third incubation comprises culturing the second aggregate in a media, e.g., DD10 media, comprising glucose and a serum-free differentiation supplement on days 10, 11, and 12.

In some embodiments, (a) the culturing the second aggregate in the media comprising glucose comprises culturing the second aggregate in the media comprising glucose on or about days 6-10 and 12-22; and the culturing the second aggregate in the media comprising sodium lactate comprises culturing the second aggregate in the media comprising sodium lactate on or about days 10-12; or (b) the culturing the second aggregate in the media comprising glucose comprises culturing the second aggregate in the media comprising glucose on or about days 4-8 and 12-22; and the culturing the second aggregate in the media comprising sodium lactate comprises culturing the second aggregate in the media comprising sodium lactate on or about days 8-12; or (c) the culturing the second aggregate in the media comprising glucose comprises culturing the second aggregate in the media comprising glucose on or about days 5-9 and 13-22; and the culturing the second aggregate in the media comprising sodium lactate comprises culturing the second aggregate in the media comprising sodium lactate on or about days 9-13; or (d) the culturing the second aggregate in the media comprising glucose comprises culturing the second aggregate in the media comprising glucose on or about days 7-11 and 13-22; and the culturing the second aggregate in the media comprising sodium lactate comprises culturing the second aggregate in the media comprising sodium lactate on or about days 11-13; or (e) the culturing the second aggregate in the media comprising glucose comprises culturing the second aggregate in the media comprising glucose on or about days 8-12 and 14-22; and the culturing the second aggregate in the media comprising sodium lactate comprises culturing the second aggregate in the media comprising sodium lactate on or about days 12-14.

In some embodiments, the culturing the second aggregate in the media comprising glucose comprises culturing the second aggregate in the media comprising glucose on or about days 6-10 and 12-22; and the culturing the second aggregate in the media comprising sodium lactate comprises culturing the second aggregate in the media comprising sodium lactate on or about days 10-12. In some embodiments, the culturing the second aggregate in the media comprising glucose comprises culturing the second aggregate in the media comprising glucose on or about days 4-8 and 12-22; and the culturing the second aggregate in the media comprising sodium lactate comprises culturing the second aggregate in the media comprising sodium lactate on or about days 8-12. In some embodiments, the culturing the second aggregate in the media comprising glucose comprises culturing the second aggregate in the media comprising glucose on or about days 5-9 and 13-22; and the culturing the second aggregate in the media comprising sodium lactate comprises culturing the second aggregate in the media comprising sodium lactate on or about days 9-13. In some embodiments, the culturing the second aggregate in the media comprising glucose comprises culturing the second aggregate in the media comprising glucose on or about days 7-11 and 13-22; and the culturing the second aggregate in the media comprising sodium lactate comprises culturing the second aggregate in the media comprising sodium lactate on or about days 11-13. In some embodiments, the culturing the second aggregate in the media comprising glucose comprises culturing the second aggregate in the media comprising glucose on or about days 8-12 and 14-22; and the culturing the second aggregate in the media comprising sodium lactate comprises culturing the second aggregate in the media comprising sodium lactate on or about days 12-14.

In some embodiments, culturing the second aggregate in the media comprising sodium lactate comprises culturing the second aggregate in the media comprising sodium lactate on one or more of any of about days 9-13. In some embodiments, culturing the second aggregate in the media comprising sodium lactate comprises culturing the second aggregate in the media comprising sodium lactate on one or more of days 10-12. In some embodiments, culturing the second aggregate in the media comprising sodium lactate comprises culturing the second aggregate in the media comprising sodium lactate on or about days 10-12.

In some embodiments, the third incubation continues until the population of cardiomyocytes are harvested. In some embodiments, the third incubation begins on or about any one of days 4 to 8, e.g., day 4, 5, 6, 7, or 8, and continues until the population of cardiomyocytes are harvested. In some embodiments, the third incubation begins on or about day 4 or day 5. In some embodiments, the third incubation begins on or about any one of days 6 to 8, e.g., day 6, 7, or 7, and continues until the population of cardiomyocytes are harvested. In some embodiments, the third incubation begins on or about day 6 or day 7. In some embodiments, the third incubation begins on or about day 6.

In some embodiments, the population of cardiomyocytes comprises a frequency of one or more cardiomyocyte markers that is or is at least 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, or 98% on one or more of any of days 8, 9, 10, 11, and 12 and/or on the sixth day following the day of the contacting with the dissociating agent. Examples of cardiomyocyte markers include, e.g., the proteins NKX2.5, cTNT, ACTN2, TNNI1, TNNI3, MYH6, MYH7, MYL2, and MYL7, NKX2.5 is NK2 homeobox 5 (also known as NKX2-5) and is encoded by the NKX2-5 gene (NM_001166175; NCBI Gene ID 1482), cTNT is troponin T2 cardiac type (also known as TNNT2) and is encoded by the TNNT2 gene (NG_007556; NCBI Gene ID 7139). ACTN2 is actinin alpha 2 and is encoded by the ACTN2 gene (NCBI Accession: NG_009081: NCBI Gene ID 88). TNNI1 is troponin I1 slow skeletal type (also known as SSTNII) and is encoded by the TNNI1 gene (NG_016649: NCBI Gene ID 7135). TNNI3 is troponin 13 cardiac type and is encoded by the TNNI3 gene (NM_000363: NCBI Gene ID 7137). MYH6 is myosin heavy chain 6 and is encoded by the MYH6 gene (NM_002471; NCBI Gene ID 4624). MYH7 is myosin heavy chain 7 and is encoded by the MYH7 gene (NM_000257; NCBI Gene ID 4625). MYL2 is myosin light chain 2 and is encoded by the MYL2 gene (NM_000432; NCBI Gene ID 4633). MYL7 is myosin light chain 7 and is encoded by the MYL2 gene (NM_021223; NCBI Gene ID 58498).

In some embodiments, the one or more cardiomyocyte markers are selected from the group consisting of NKX2.5, cTNT. ACTN2. TNNI1, TNNI3, MYH6, MYH7, MYL2, and MYL7. In some embodiments, the one or more cardiomyocyte markers comprise NKX2.5 and cTNT. In some embodiments, the one or more cardiomyocyte markers comprise MYH6 and MYL7. In some embodiments, the one or more cardiomyocyte markers comprise NKX2.5, cTNT, MYH6, and MYL7.

In some embodiments, during the third incubation, the population of cardiomyocytes comprises a frequency of NKX2.5+/cTNT+ cells that is or is at least 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, or 98% on one or more of any of days 8-12, i.e., any one or more of days 8, 9, 10, 11, and 12, and/or on the sixth day following the day of the contacting with the dissociating agent. In some embodiments, the frequency of NKX2.5+/cTNT+ cells is or is at least 85%, 86%, 87%, 88%, 89%, 90%, 91%, 92%, 93%, 94%,95%,96%,97%, or 98% on one or more of any of days 8-12, i.e., any one or more of days 8, 9, 10, 11, and 12, and/or on the sixth day following the day of the contacting with the dissociating agent. In some embodiments, during the third incubation, the population of cardiomyocytes comprises a frequency of NKX2.5+/cTNT+ cells that is or is at least 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, or 98% on one or more of any of days 9-11, i.e., any one or more of days 9, 10, and 11, and/or on the sixth day following the day of the contacting with the dissociating agent. In some embodiments, the frequency of NKX2.5+/cTNT+ cells is or is at least 85%, 86%, 87%, 88%, 89%, 90%, 91%, 92%, 93%, 94%, 95%, 96%, 97%, or 98% on one or more of any of days 9-11. i.e., any one or more of days 9, 10, and 11, and/or on the sixth day following the day of the contacting with the dissociating agent. In some embodiments, during the third incubation, the population of cardiomyocytes comprises a frequency of NKX2.5+/cTNT+ cells that is or is at least 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, or 98% on or about day 10 and/or on the sixth day following the day of the contacting with the dissociating agent. In some embodiments, the frequency of NKX2.5+/cTNT+ cells is or is at least 85%, 86%, 87%, 88%, 89%, 90%, 91%, 92%, 93%, 94%, 95%, 96%, 97%, or 98% on or about day 10 and/or on the sixth day following the day of the contacting with the dissociating agent. In some embodiments, the frequency of NKX2.5+/cTNT+ cells is or is at least 85%, 86%, 87%, 88%, 89%, 90%, 91%, 92%, 93%, 94%, 95%, 96%, 97%, or 98% on or about day 10.

In some embodiments, the frequency of NKX2.5+/cTNT+ cells is or is at least 90%, 91%, 92%, 93%, 94%, 95%, 96%, 97%, or 98% on one or more of any of days 8-12. i.e., any one or more of days 8, 9, 10, 11, and 12, and/or on the sixth day following the day of the contacting with the dissociating agent. In some embodiments, the frequency of NKX2.5+/cTNT+ cells is or is at least 90%, 91%, 92%, 93%, 94%, 95%, 96%, 97%, or 98% on one or more of any of days 9-11. i.e., on one or more of any of days 9, 10, and 11, and/or on the sixth day following the day of the contacting with the dissociating agent. In some embodiments, the frequency of NKX2.5+/cTNT+ cells is or is at least 90%, 91%, 92%, 93%, 94%, 95%, 96%, 97%, or 98% on or about day 10 and/or on the sixth day following the day of the contacting with the dissociating agent.

In some embodiments, the third incubation occurs in suspension. In some embodiments, the third incubation comprises culturing the second aggregate in suspension. In some embodiments, the third incubation comprises culturing the second aggregate in suspension beginning on or about day 4, 5, 6, 7, 8, 9, or 10. In some embodiments, the third incubation comprises culturing the second aggregate in suspension beginning on or about day 6. In some embodiments, the third incubation comprises culturing the second aggregate in suspension beginning at or about 1 day, 2 days, 3 days, or 4 days after the contacting with the dissociating agent. In some embodiments, the third incubation comprises culturing the second aggregate in suspension beginning at or about 2 days after the contacting with the dissociating agent.

### Population of Cardiomyocytes

In some embodiments, the population of cardiomyocytes comprises a frequency of one or more cardiomyocyte markers that is or is at least 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, or 98%. In some embodiments, the one or more cardiomyocyte markers are selected from the group consisting of NKX2.5, cTNT, ACTN2, TNNI1, TNNI3, MYH6, MYH7, MYL2, and MYL7. In some embodiments, the one or more cardiomyocyte markers comprise NKX2.5 and cTNT. In some embodiments, the one or more cardiomyocyte markers comprise MYH6 and MYL7. In some embodiments, the one or more cardiomyocyte markers comprise NKX2.5, cTNT, MYH6, and MYL7.

In some embodiments, the population of cardiomyocytes comprises a frequency of one or more cardiomyocyte markers selected from the group consisting of NKX2.5, cTNT, ACTN2, TNNI1, TNNI3, MYH6, MYH7, MYL2, and MYL7 that is or is at least 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, or 98%. In some embodiments, the population of cardiomyocytes comprises a frequency of NKX2.5+/cTNT+ that is or is at least 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, or 98%. In some embodiments, the population of cardiomyocytes comprises a frequency of MYH6+/MYL7+ that is or is at least 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, or 98%. In some embodiments, the population of cardiomyocytes comprises a frequency of NKX2.5+/cTNT+/MYH6+/MYL7+ that is or is at least 75%, 76%, 77%, 78%, 79%80%, 81%, 82%, 83%, 84%, 85%, 86%, 87%, 88%, 89%, 90%, 91%, 92%, 93%, 94%, 95%, 96%, 97%, or 98%. In some embodiments, the population of cardiomyocytes comprises a frequency of NKX2.5+/cTNT+ that is or is at least 85%, 86%, 87%, 88%, 89%, 90%, 91%, 92%, 93%, 94%, 95%, 96%, 97%, or 98%. In some embodiments, the population of cardiomyocytes comprises a frequency of MYH6+/MYL7+ that is or is at least 85%, 86%, 87%, 88%, 89%, 90%, 91%, 92%, 93%, 94%, 95%, 96%, 97%, or 98%. In some embodiments, the population of cardiomyocytes comprises a frequency of NKX2.5+/cTNT+/MYH6+/MYL7+ that is or is at least 85%, 86%, 87%, 88%, 89%, 90%, 91%, 92%, 93%, 94%, 95%, 96%, 97%, or 98%.

In some embodiments, the population of cardiomyocytes in the second aggregate comprises a frequency of mature cardiomyocytes that is higher as compared to a reference population of cardiomyocytes in an aggregate comprising cells that were not previously dissociated. In some embodiments, on or about day 20, 21, 22, 23, or 24, the population of cardiomyocytes in the second aggregate comprises a frequency of mature cardiomyocytes that is higher as compared to a reference population of cardiomyocytes in an aggregate comprising cells that were not previously dissociated. In some embodiments, on or about day 22, the population of cardiomyocytes in the second aggregate comprises a frequency of mature cardiomyocytes that is higher as compared to a reference population of cardiomyocytes in an aggregate comprising cells that were not previously dissociated.

In some embodiments, the reference population of cardiomyocytes is differentiated under the same or substantially the same conditions as the population of cardiomyocytes in the second aggregate except for b) and c). In some embodiments, the reference population of cardiomyocytes is differentiated under conditions that do not comprise contacting the aggregate with a dissociating agent to form a population of dissociated cells. In some embodiments, the reference population of cardiomyocytes is differentiated under the same or substantially the same conditions as the population of cardiomyocytes in the second aggregate except that it does not comprise contacting the aggregate with a dissociating agent. In some embodiments, the reference population of cardiomyocytes is differentiated under the same or substantially the same conditions as the population of cardiomyocytes in the second aggregate except that it does not comprise contacting the aggregate with a dissociating agent. In some embodiments, the reference population of cardiomyocytes had not been previous dissociated using a dissociating agent.

In some embodiments, the frequency of mature cardiomyocytes is based on a frequency of the presence of one or more mature cardiomyocyte markers in the population of cardiomyocytes in the second aggregate. The cardiomyocyte markers can be any marker, e.g., any cell surface marker, used to identify cardiomyocytes. In some embodiments, the one or more cardiomyocyte markers comprises one or more markers selected from the group consisting of NKX2.5, cTNT, ACTN2, TNNI1, TNNI3, MYH6, MYH7, MYL2, and MYL7. In some embodiments, the one or more cardiomyocyte markers comprise MYH6 and MYL7. In some embodiments, the one or more cardiomyocyte markers comprise NKX2.5, cTNT, MYH6, and MYL7. In some embodiments, the one or more cardiomyocyte markers comprise NKX2.5 and cTNT.

### Modifications for Reducing Engraftment Arrhythmia (AE)

In some embodiments, the population of cardiomyocytes comprise one or more modifications that reduce or prevent engraftment arrhythmia (EA) when grafted into a subject. In some embodiments, the population of dissociated cells comprise one or more modifications that reduce or prevent EA when grafted into a subject. In some embodiments, the population of cells in the second aggregate comprise one or more modifications that reduce or prevent EA when grafted into a subject.

In some embodiments, the one or more modifications comprise one or more modifications that (i) increase expression of one or more tolerogenic factors; and/or (ii) reduce expression of one or more major histocompatibility complex (MHC) class I molecules and/or MHC class II molecules, relative to a population of cardiomyocytes that do not comprise the one or more modifications.

In some embodiments, the one or more modifications comprise one or more modifications that (a) reduce expression of one or more of CACNA1G, HCN4, and SLC8A1; and/or (b) increase expression of one or more of KCNJ2, TRDN, SRL, HRC, and CASQ2.

In some embodiments, the one or more modifications comprise one or more modifications that (a) reduce expression of one or more of CACNA1H, HCN4, and SLC8A1; and/or (b) increase expression of one or more of KCNJ2, TRDN, SRL, HRC, and CASQ2.

In some embodiments, the one or more modifications comprise one or more modifications that (a) reduce expression of CACNA1H, HCN4, and SLC8A1; and (b) increase expression of KCNJ2.

Exemplary methods to introduce modifications into a cell to alter expression are known and are also described herein. For instance, any of a variety of methods for overexpressing or increasing expression of a gene or protein may be used, such as by introduction or delivery of an exogenous polynucleotide encoding a protein (i.e. a transgene) or introduction of delivery of a fusion protein of a DNA-targeting domain and a transcriptional activator targeting a gene. Also, any of a variety of methods for reducing or eliminating expression of a gene or protein may be used, including non-gene editing methods such as by introduction or delivery of inhibitory nucleic acids (e.g. RNAi) or gene editing methods involving introduction or delivery of a targeted nuclease system (e.g. CRISPR/Cas). In some embodiments, the method for reducing or eliminating expression is via a nuclease-based gene editing technique.

In some embodiments, genome editing technologies utilizing rare-cutting endonucleases (e.g., the CRISPR/Cas, TALEN, zinc finger nuclease, meganuclease, and homing endonuclease systems) are used to reduce or eliminate expression of genes, including immune genes (e.g., by deleting genomic DNA of critical immune genes) in human cells. In some embodiments, the genome editing technology comprises use of nickases, base editing, prime editing, and gene writing.

In certain embodiments, genome editing technologies or other gene modulation technologies are used to: insert one or more of the KCNJ2, TRDN, SRL, HRC, and CASQ2 genes: reduce or eliminate expression of one or more of the CACNA1G, CACNA1H, HCN4, and SLC8A1 genes; or any combination thereof, thus producing engineered cells, e.g., an engineered population of cardiomyocytes, that can result in reduced or eliminated EA following engraftment in a subject.

Therefore, the cells provided herein, e.g., population of cardiomyocytes, or population of dissociated cells, or populations of cells in the second aggregate, can, in some embodiments, exhibit modulated expression (e.g., reduced or eliminated expression) of one or more of CACNA1G, CACNA1H, HCN4, and SLC8A1, and/or modulated expression (e.g., increased expression or overexpression) of one or more of KCNJ2, TRDN, SRL, HRC, and CASQ2. In some embodiments, the cells provided herein, e.g., population of cardiomyocytes, or population of dissociated cells, or populations of cells in the second aggregate, exhibit modulated expression (e.g., reduced or eliminated expression) of CACNA1G, HCN4, and SLC8A1, and modulated expression (e.g., increased expression or overexpression) of KCNJ2.

In some embodiments, the cells provided herein, e.g., population of cardiomyocytes, or population of dissociated cells, or populations of cells in the second aggregate, do not cause engraftment arrhythmia following engrafiment in a subject.

Methods for reducing expression of a target gene of interest are known. Any method for reducing expression of a target polynucleotide may be used. In some embodiments, the modifications (e.g., genetic modifications) result in permanent elimination or reduction in expression of the target polynucleotide. For instance, in some embodiments, the target polynucleotide or gene is disrupted by introducing a DNA break in the target polynucleotide, such as by using a targeting endonuclease. In other embodiments, the modifications (e.g., genetic modifications) result in transient reduction in expression of the target polynucleotide. For instance, in some embodiments gene repression is achieved using an inhibitory nucleic acid that is complementary to the target polynucleotide to selectively suppress or repress expression of the gene, for instance using antisense techniques, such as by RNA interference (RNAi), short interfering RNA (siRNA), short hairpin (shRNA), and/or ribozymes.

In some embodiments, the target polynucleotide sequence is a genomic sequence. In some embodiments, the target polynucleotide sequence is a human genomic sequence. In some embodiments, the target polynucleotide sequence is a mammalian genomic sequence. In some embodiments, the target polynucleotide sequence is a vertebrate genomic sequence. In some embodiments, gene disruption is carried out by induction of one or more double-stranded breaks and/or one or more single-stranded breaks in the gene, typically in a targeted manner. In some embodiments, the double-stranded or single-stranded breaks are made by a nuclease, e.g., an endonuclease, such as a gene-targeted nuclease. In some embodiments, the targeted nuclease is selected from zinc finger nucleases (ZFN), transcription activator-like effector nucleases (TALENs), and RNA-guided nucleases such as a CRISPR-associated nuclease (Cas), specifically designed to be targeted to the sequence of a gene or a portion thereof. In some embodiments, the targeted nuclease generates double-stranded or single-stranded breaks that then undergo repair through error prone non-homologous end joining (NHEJ) or, in some cases, precise homology directed repair (HDR) in which a template is used. In some embodiments, the targeted nuclease generates DNA double strand breaks (DSBs). In some embodiments, the process of producing and repairing the breaks is typically error prone and results in insertions and deletions (indels) of DNA bases from NHEJ repair. In some embodiments, the genetic modification may induce a deletion, insertion or mutation of the nucleotide sequence of the target gene. In some cases, the genetic modification may result in a frameshift mutation, which can result in a premature stop codon. In examples of nuclease-mediated gene editing the targeted edits occur on both alleles of the gene resulting in a biallelic disruption or edit of the gene. In some embodiments, all alleles of the gene are targeted by the gene editing. In some embodiments, genetic modification with a targeted nuclease, such as using a CRISPR/Cas system, leads to complete knockout of the gene.

### Harvesting and Cryopreservation

In some embodiments, the method further comprises harvesting the population of cardiomyocytes. In some embodiments, the method further comprises harvesting the population of dissociated cells. In some embodiments, the method further comprises harvesting the second aggregate.

In some embodiments, the population of cardiomyocytes is harvested at any suitable time, e.g., at any suitable during the third incubation or beyond. In some embodiments, the population of cardiomyocytes is harvested based on a frequency of one or more cardiomyocyte markers. In some embodiments, the population of cardiomyocytes is harvested based on criteria comprising a frequency of one or more cardiomyocyte markers being above a threshold. In some embodiments, the threshold is at or above 85%, 96%, 97%, 88%, 89%, 90%, 91%, 92%, 93%, 94%, 95%, 96%, 97%, 98%, or 99%.

In some embodiments, the population of cardiomyocytes is harvested on or about day 14, 15, 16, 17, 18, 19, 20, 21, 22, 23, or 24. In some embodiments, the population of cardiomyocytes is harvested on or about day 20, 21, 22, 23, or 24. In some embodiments, the population of cardiomyocytes is harvested on or about day 21, 22, or 23. In some embodiments, the population of cardiomyocytes is harvested on or about day 22.

In some embodiments, the population of dissociated cells is harvested on or about day 2, 3, 4, 5, or 6. In some embodiments, the population of dissociated cells is harvested on or about day 3, 4, or 5. In some embodiments, the population of dissociated cells is harvested on or about day 4 or day 5. In some embodiments, the population of dissociated cells is harvested on or about day 4.

In some embodiments, the second aggregate is harvested on or about any one of days 3 to 22. In some embodiments, the second aggregate is harvested on or about any one of days 3, 4, 5, 6, 7, 8, 9, 10, 11, 12, 13, 14, or 15. In some embodiments, the second aggregate is harvested on or about any one of days 4, 5, 6, 7, 8, 9, 10, 11, or 12. In some embodiments, the second aggregate is harvested on or about day 4, 5, 6, 7, 8, 9, or 10. In some embodiments, the second aggregate is harvested on or about day 5, 6, or 7.

The harvesting, e.g., the harvesting of the population of cardiomyocytes, the population of the population of dissociated cells, and/or the second aggregate, can be performed using any suitable method, including any known method, such as any method as described or used in, e.g., Fujiwara et al., PLoS One. (2001) 6(2):e16734; Dambrot et al., Biochem J. (2011) 434(1):25-35; Foldes et al., J Mol Cell Cardiol. (2011)50(2):367-76; Wang et al., Sci China Life Sci. (2010) 53(5):581-9; Chen et al., J Cell Biochem. (2010) (1):29-39; Yang et al., Nature (2008) 453:524-28; Kattman et al., Cell Stem Cell (2011) 8:228-40; Laflamme et al., Nat. Biotechnol. (2007) 25: 1015-24: Paige et al., PLoS One (2010) 5(6): e11134; Xu et al., Regen Med (2011) 6(1):53-66; Mignone et al., Circ J (2010) 74(12):2517-26; Takei et al., Am J Physiol Heart Circ Physiol. (2009) 296(6):H1793-803; WO2013013206; and WO2013056072.

In some embodiments, the harvesting of the population of cardiomyocytes comprises centrifuging the population of cardiomyocytes into a pellet. In some embodiments, the harvesting of the population of cardiomyocytes further comprises one or more washing steps. In some embodiments, the method further comprises cryopreserving the harvested population of cardiomyocytes.

In some embodiments, the harvesting of the population of dissociated cells comprises centrifuging the population of dissociated cells into a pellet. In some embodiments, the harvesting of the population of dissociated cells further comprises one or more washing steps. In some embodiments, the method further comprises cryopreserving the harvested population of dissociated cells.

In some embodiments, the harvesting of the second aggregate comprises centrifuging the second aggregate into a pellet. In some embodiments, the harvesting of the second aggregate further comprises one or more washing steps. In some embodiments, the method further comprises cryopreserving the harvested second aggregate.

The cryopreserving can be performed using any suitable method, including any known method.

By using cardiomyocytes, large-scale, effective cell-based therapies can be produced to combat various heart diseases or conditions.

Genome-edited cardiomyocyte cells may be used to treat or prevent a disease in a subject (for example, as described in WO2022187379A1 the contents of which are incorporated herein by reference in their entirety). A method may include administering to the subject a composition that includes the genome-edited cardiomyocytes described herein or produced by the method described herein. The disease could include, for example, heart diseases or heart conditions.

A genome-edited cardiomyocytes may be administered to a subject alone or in combination with one or more other therapies. For example, a genome-edited cardiomyocytes may be administered to a subject in combination a pharmaceutical composition that includes the active agent and a pharmaceutically acceptable carrier and/or in combination with a cellular therapy. The cardiomyocyte cell may be administered to a patient, preferably a mammal, and more preferably a human, in an amount effective to produce the desired effect. The cardiomyocyte cell may be administered in a variety of routes, including, for example, intravenously, intratumorally, intraarterially, transdermally, via local delivery by catheter or stent, via a needle or other device for intratumoral injection, subcutaneously, etc. The cardiomyocyte cell may be administered once or multiple times. A physician having ordinary skill in the art may determine and prescribe the effective amount and dosing of an adaptive cardiomyocyte cell and, optionally, the pharmaceutical composition required.

The heart disease or heart condition may include, for example, pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, other cardiomyopathy, myocarditis, myocardial infarction, myocardial ischemic reperfusion injury, ventricular dysfunction, heart failure, congestive heart failure, coronary artery disease, end stage heart disease, atherosclerosis, ischemia, hypertension, restenosis, angina pectoris, rheumatic heart, arterial inflammation, or cardiovascular disease. In some embodiments, the heart disease or condition is myocardial infarction (MI).

In any of the provided embodiments, the subject administered the cardiomyocyte cell therapy has a condition or disease, such as a heart condition or disease. In some embodiments, the heart condition or disease is selected from the group consisting of pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, other cardiomyopathy, myocarditis, myocardial infarction (MI), myocardial ischemic reperfusion injury, ventricular dysfunction, heart failure, congestive heart failure, coronary artery disease, end stage heart disease, atherosclerosis, ischemia, hypertension, restenosis, angina pectoris, rheumatic heart, arterial inflammation, or cardiovascular disease. In some embodiments, the heart condition or disease is myocardial infarction (MI). Thus, in some embodiments, the cardiomyocyte cell therapy is administered to a subject to treat a MI (e.g. as a composition comprising cardiomyocytes).

### H. Neural Cells

Neural cells to be used in a cell therapy product may be profiled for donor capability at any stage of the manufacturing process of the cell therapy product.

Neural cells used in a cell therapy product may be primary neural cells. Methods for profiling a population of cells for donor capability as described anywhere herein may be performed on primary (e.g., genome-edited) neural cells.

As described elsewhere herein, neural cells used in a cell therapy product may be pluripotent stem cell (iPSC)-derived neural cells. Methods for profiling a population of cells for donor capability as described anywhere herein may also be performed on stem cells capable of differentiating to form neural cells. Methods for profiling a population of cells for donor capability as described anywhere herein may also be performed on stem cell derived (e.g., genome-edited) neural cells.

Relevant information concerning neural cells as referred to in the context of the present disclosure is known in the art, including certain information regarding desired features of neural cells when used for cell therapy. It will be understood that embodiments concerning neural cells described herein may be readily and appropriately combined with embodiments describing HIP cells (e.g., exhibiting reduced expression of one or more molecules of the MHC class I and/or MHC class II molecules and increased expression of at least one tolerogenic factor), as well as embodiments describing safety switches, and other modified/gene edited cells as described herein. Neural cells to be used in a cell therapy product may be profiled for donor capability at any stage of the of the editing process during manufacturing of the cell therapy product.

The neural cells described herein may be used to treat or prevent a disease in a subject.

Provided herein are different neural cell types differentiated from engineered pluripotent cells (e.g., iPSCs) as described that are useful for subsequent transplantation or engraftment into recipient subjects. As will be appreciated by those in the art, the methods for differentiation depend on the desired cell type using known techniques. Exemplary neural cell types include, but are not limited to, cerebral endothelial cells, neurons (e.g., dopaminergic neurons), glial cells, and the like.

In some embodiments, differentiation of induced pluripotent stem cells is performed by exposing or contacting cells to specific factors which are known to produce a specific cell lineage(s), so as to target their differentiation to a specific, desired lineage and/or cell type of interest. In some embodiments, terminally differentiated cells display specialized phenotypic characteristics or features. In certain embodiments, the stem cells described herein are differentiated into a neuroectodermal, neuronal, neuroendocrine, dopaminergic, cholinergic, serotonergic (5-HT), glutamatergic, GABAergic, adrenergic, noradrenergic, sympathetic neuronal, parasympathetic neuronal, sympathetic peripheral neuronal, or glial cell population. In some instances, the glial cell population includes a microglial (e.g., amoeboid, ramified, activated phagocytic, and activated non-phagocytic) cell population or a macroglial (central nervous system cell: astrocyte, oligodendrocyte, ependymal cell, and radial glia; and peripheral nervous system cell: Schwann cell and satellite cell) cell population, or the precursors and progenitors of any of the preceding cells.

Protocols for generating different types of neural cells are described in PCT Application No. WO2010144696, U.S. Pat. Nos. 9,057,053; 9,376,664; and 10,233,422. Additional descriptions of methods for differentiating hypoimmunogenic pluripotent cells can be found, for example, in Deuse et al., Nature Biotechnology, 2019, 37, 252-258 and Han et al., Proc Natl Acad Sci USA. 2019, 116(21), 10441-10446. Methods for determining the effect of neural cell transplantation in an animal model of a neurological disorder or condition are described in the following references: for spinal cord injury—Curtis et al., Cell Stem Cell, 2018, 22, 941-950; for Parkinson's disease—Kikuchi et al., Nature, 2017, 548:592-596; for ALS—Izrael et al., Stem Cell Research, 2018, 9(1):152 and Izrael et al., IntechOpen, DOI: 10.5772/intechopen.72862; for epilepsy—Upadhya et al., PNAS, 2019, 116(1):287-296.

In some embodiments, the population of engineered neural cells, such as neural cells differentiated from iPSCs derived from one or more individual donors (e.g., healthy donors), are maintained in culture, in some cases expanded, prior to administration. In certain embodiments, the population of neural cells are cryopreserved prior to administration.

In some embodiments, the present technology is directed to engineered neural cells, such as neural cells differentiated from iPSCs derived from one or more individual donors (e.g., healthy donors), that overexpress a tolerogenic factor (e.g., CD47), and have reduced expression or lack expression of one or more MHC class I molecules and/or one or more MHC class II molecules (e.g., one or more MHC class I human leukocyte antigen molecules and/or one or more MHC class II human leukocyte antigen molecules).

In some embodiments, the provided engineered neural cells evade immune recognition. In some embodiments, the engineered neural cells described herein, such as neural cells differentiated from iPSCs derived from one or more individual donors (e.g., healthy donors), do not activate an immune response in the patient (e.g., recipient upon administration). Provided are methods of treating a disease by administering a population of engineered neural cells described herein to a subject (e.g., recipient) or patient in need thereof.

In some embodiments, neural cells are administered to a subject to treat Parkinson's disease, Huntington disease, multiple sclerosis, other neurodegenerative disease or condition, attention deficit hyperactivity disorder (ADHD), Tourette Syndrome (TS), schizophrenia, psychosis, depression, other neuropsychiatric disorder. In some embodiments, neural cells described herein are administered to a subject to treat or ameliorate stroke. In some embodiments, the neurons and glial cells are administered to a subject with amyotrophic lateral sclerosis (ALS).

### 1) Cerebral Endothelial Cells

Cerebral endothelial cells to be used in a cell therapy product may be profiled for donor capability at any stage of the manufacturing process of the cell therapy product.

Cerebral endothelial cells used in a cell therapy product may be primary cerebral endothelial cells. Methods for profiling a population of cells for donor capability as described anywhere herein may be performed on primary (e.g., genome-edited) cerebral endothelial cells.

As described elsewhere herein, cerebral endothelial cells used in a cell therapy product may be pluripotent stem cell (iPSC)-derived cerebral endothelial cells. Methods for profiling a population of cells for donor capability as described anywhere herein may also be performed on stem cells capable of differentiating to form cerebral endothelial cells. Methods for profiling a population of cells for donor capability as described anywhere herein may also be performed on stem cell derived (e.g., genome-edited) cerebral endothelial cells.

Relevant information concerning cerebral endothelial cells as referred to in the context of the present disclosure is known in the art, including certain information regarding desired features of cerebral endothelial cells when used for cell therapy. It will be understood that embodiments concerning cerebral endothelial cells described herein may be readily and appropriately combined with embodiments describing HIP cells (e.g., exhibiting reduced expression of one or more molecules of the MHC class I and/or MHC class II molecules and increased expression of at least one tolerogenic factor), as well as embodiments describing safety switches, and other modified/gene edited cells as described herein. Cerebral endothelial cells to be used in a cell therapy product may be profiled for donor capability at any stage of the editing process during manufacturing of the cell therapy product.

In some embodiments, cerebral endothelial cells (ECs), precursors, and progenitors thereof are differentiated from pluripotent stem cells (e.g., induced pluripotent stem cells) on a surface by culturing the cells in a medium comprising one or more factors that promote the generation of cerebral ECs or neural cell. In some instances, the medium includes one or more of the following: CHIR-99021, VEGF, basic FGF (bFGF), and Y-27632. In some embodiments, the medium includes a supplement designed to promote survival and functionality for neural cells.

In some embodiments, cerebral endothelial cells (ECs), precursors, and progenitors thereof are differentiated from pluripotent stem cells on a surface by culturing the cells in an unconditioned or conditioned medium. In some instances, the medium comprises factors or small molecules that promote or facilitate differentiation. In some embodiments, the medium comprises one or more factors or small molecules selected from the group consisting of VEGR, FGF, SDF-1, CHIR-99021, Y-27632, SB 431542, and any combination thereof. In some embodiments, the surface for differentiation comprises one or more extracellular matrix proteins. The surface can be coated with the one or more extracellular matrix proteins. The cells can be differentiated in suspension and then put into a gel matrix form, such as matrigel, gelatin, or fibrin/thrombin forms to facilitate cell survival. In some cases, differentiation is assayed as is known in the art, generally by evaluating the presence of cell-specific markers.

In some embodiments, the cerebral endothelial cells express or secrete a factor selected from the group consisting of CD31, VE cadherin, and a combination thereof. In certain embodiments, the cerebral endothelial cells express or secrete one or more of the factors selected from the group consisting of CD31, CD34, CD45. CD 117 (c-kit), CD146, CXCR4, VEGF, SDF-1, PDGF, GLUT-1, PECAM-1, eNOS, claudin-5, occludin, ZO-1, p-glycoprotein, von Willebrand factor, VE-cadherin, low density lipoprotein receptor LDLR, low density lipoprotein receptor-related protein 1 LRP1, insulin receptor INSR, leptin receptor LEPR, basal cell adhesion molecule BCAM, transferrin receptor TFRC, advanced glycation endproduct-specific receptor AGER, receptor for retinol uptake STRA6, large neutral amino acids transporter small subunit 1 SLC7A5, excitatory amino acid transporter 3 SLC1A1, sodium-coupled neutral amino acid transporter 5 SLC38A5, solute carrier family 16 member 1 SLC16A1, ATP-dependent translocase ABCB1, ATP-ABCC2-binding cassette transporter ABCG2, multidrug resistance-associated protein 1 ABCC1, canalicular multispecific organic anion transporter 1 ABCC2, multidrug resistance-associated protein 4 ABCC4, and multidrug resistance-associated protein 5 ABCC5.

In some embodiments, the cerebral ECs are characterized with one or more of the features selected from the group consisting of high expression of tight junctions, high electrical resistance, low fenestration, small perivascular space, high prevalence of insulin and transferrin receptors, and high number of mitochondria.

In some embodiments, cerebral ECs are selected or purified using a positive selection strategy. In some instances, the cerebral ECs are sorted against an endothelial cell marker such as, but not limited to, CD31. In other words, CD31 positive cerebral ECs are isolated. In some embodiments, cerebral ECs are selected or purified using a negative selection strategy. In some embodiments, undifferentiated or pluripotent stem cells are removed by selecting for cells that express a pluripotency marker including, but not limited to, TRA-1-60 and SSEA-1.

In some embodiments, cerebral endothelial cells are administered to alleviate the symptoms or effects of cerebral hemorrhage. In some embodiments, dopaminergic neurons are administered to a patient with Parkinson's disease. In some embodiments, noradrenergic neurons, GABAergic interneurons are administered to a patient who has experienced an epileptic seizure. In some embodiments, motor neurons, interneurons, Schwann cells, oligodendrocytes, and microglia are administered to a patient who has experienced a spinal cord injury.

### 2) Dopaminergic Neurons

Dopaminergic neurons to be used in a cell therapy product may be profiled for donor capability at any stage of the manufacturing process of the cell therapy product.

Dopaminergic neurons used in a cell therapy product may be primary dopaminergic neurons. Methods for profiling a population of cells for donor capability as described anywhere herein may be performed on primary (e.g., genome-edited) dopaminergic neurons.

As described elsewhere herein, dopaminergic neurons used in a cell therapy product may be pluripotent stem cell (iPSC)-derived dopaminergic neurons. Methods for profiling a population of cells for donor capability as described anywhere herein may also be performed on stem cells capable of differentiating to form dopaminergic neurons. Methods for profiling a population of cells for donor capability as described anywhere herein may also be performed on stem cell derived (e.g., genome-edited) dopaminergic neurons.

Relevant information concerning dopaminergic neurons as referred to in the context of the present disclosure is known in the art, including certain information regarding desired features of dopaminergic neurons when used for cell therapy. It will be understood that embodiments concerning dopaminergic neurons described herein may be readily and appropriately combined with embodiments describing HIP cells (e.g., exhibiting reduced expression of one or more molecules of the MHC class I and/or MHC class II molecules and increased expression of at least one tolerogenic factor), as well as embodiments describing safety switches, and other modified/gene edited cells as described herein. Dopaminergic neurons to be used in a cell therapy product may be profiled for donor capability at any stage of the editing process during manufacturing of the cell therapy product.

In some embodiments, HIP cells described herein are differentiated into dopaminergic neurons include neuronal stem cells, neuronal progenitor cells, immature dopaminergic neurons, and mature dopaminergic neurons.

In some cases, the term “dopaminergic neurons” includes neuronal cells which express tyrosine hydroxylase (TH), the rate-limiting enzyme for dopamine synthesis. In some embodiments, dopaminergic neurons secrete the neurotransmitter dopamine, and have little or no expression of dopamine hydroxylase. A dopaminergic (DA)neuron can express one or more of the following markers: neuron-specific enolase (NSE), 1-aromatic amino acid decarboxylase, vesicular monoamine transporter 2, dopamine transporter, Nurr-1, and dopamine-2 receptor (D2 receptor). In certain cases, the term “neural stem cells” includes a population of pluripotent cells that have partially differentiated along a neural cell pathway and express one or more neural markers including, for example, nestin. Neural stem cells may differentiate into neurons or glial cells (e.g., astrocytes and oligodendrocytes). The term “neural progenitor cells” includes cultured cells which express FOXA2 and low levels of b-tubulin, but not tyrosine hydroxylase. Such neural progenitor cells have the capacity to differentiate into a variety of neuronal subtypes; for example, a variety of dopaminergic neuronal subtypes, upon culturing the appropriate factors, such as those described herein.

In some embodiments, the DA neurons derived from HIP cells are administered to a patient, e.g., human patient to treat a neurodegenerative disease or condition. In some cases, the neurodegenerative disease or condition is selected from the group consisting of Parkinson's disease, Huntington disease, and multiple sclerosis. In other embodiments, the DA neurons are used to treat or ameliorate one or more symptoms of a neuropsychiatric disorder, such as attention deficit hyperactivity disorder (ADHD), Tourette Syndrome (TS), schizophrenia, psychosis, and depression. In yet other embodiments, the DA neurons are used to treat a patient with impaired DA neurons.

In some embodiments. DA neurons, precursors, and progenitors thereof are differentiated from pluripotent stem cells by culturing the stem cells in medium comprising one or more factors or additives. Useful factors and additives that promote differentiation, growth, expansion, maintenance, and/or maturation of DA neurons include, but are not limited to, Wnt1, FGF2, FGF8, FGF8a, sonic hedgehog (SHH), brain derived neurotrophic factor (BDNF), transforming growth factor a (TGF-a), TGF-b, interleukin 1 beta, glial cell line-derived neurotrophic factor (GDNF), a GSK-3 inhibitor (e.g., CHIR-99021), a TGF-b inhibitor (e.g., SB-431542), B-27 supplement, dorsomorphin, purmorphamine, noggin, retinoic acid, cAMP, ascorbic acid, neurturin, knockout serum replacement, N-acetyl cysteine, c-kit ligand, modified forms thereof, mimics thereof, analogs thereof, and variants thereof. In some embodiments, the DA neurons are differentiated in the presence of one or more factors that activate or inhibit the WNT pathway, NOTCH pathway, SHH pathway, BMP pathway, FGF pathway, and the like. Differentiation protocols and detailed descriptions thereof are provided in, e.g., U.S. Pat. No. 9,968,637, US7.674,620, Kim et al, Nature, 2002, 418,50-56; Bjorklund et al, PNAS, 2002. 99(4), 2344-2349; Grow et al., Stem Cells Transl Med. 2016. 5(9): 1133-44, and Cho et al, PNAS, 2008. 105:3392-3397, the disclosures in their entirety including the detailed description of the examples, methods, figures, and results are herein incorporated by reference.

In some embodiments, the population of hypoimmunogenic dopaminergic neurons is isolated from non-neuronal cells. In some embodiments, the isolated population of hypoimmunogenic dopaminergic neurons are expanded prior to administration. In certain embodiments, the isolated population of hypoimmunogenic dopaminergic neurons are expanded and cryopreserved prior to administration.

To characterize and monitor DA differentiation and assess the DA phenotype, expression of any number of molecular and genetic markers can be evaluated. For example, the presence of genetic markers can be determined by various methods known to those skilled in the art. Expression of molecular markers can be determined by quantifying methods such as, but not limited to, qPCR-based assays, immunoassays, immunocytochemistry assays, immunoblotting assays, and the like. Exemplary markers for DA neurons include, but are not limited to, TH, b-tubulin, paired box protein (Pax6), insulin gene enhancer protein (Isl1), nestin, diaminobenzidine (DAB), G protein-activated inward rectifier potassium channel 2 (GIRK2), microtubule-associated protein 2 (MAP-2), NURR1, dopamine transporter (DAT), forkhead box protein A2 (FOXA2), FOX3, doublecortin, and LIM homeobox transcription factor 1-beta (LMXIB), and the like. In some embodiments, the DA neurons express one or more of the markers selected from corin, FOXA2, TuJ1, NURR1, and any combination thereof.

In some embodiments, DA neurons are assessed according to cell electrophysiological activity. The electrophysiology of the cells can be evaluated by using assays knowns to those skilled in the art. For instance, whole-cell and perforated patch clamp, assays for detecting electrophysiological activity of cells, assays for measuring the magnitude and duration of action potential of cells, and assays for detecting dopamine production of DA cells.

In some embodiments, DA neuron differentiation is characterized by spontaneous rhythmic action potentials, and high-frequency action potentials with spike frequency adaption upon injection of depolarizing current. In other embodiments, DA differentiation is characterized by the production of dopamine. The level of dopamine produced is calculated by measuring the width of an action potential at the point at which it has reached half of its maximum amplitude (spike half-maximal width).

In some embodiments, the differentiated DA neurons are transplanted either intravenously or by injection at particular locations in the patient. In some embodiments, the differentiated DA cells are transplanted into the substantia nigra (particularly in or adjacent of the compact region), the ventral tegmental area (VTA), the caudate, the putamen, the nucleus accumbens, the subthalamic nucleus, or any combination thereof, of the brain to replace the DA neurons whose degeneration resulted in Parkinson's disease. The differentiated DA cells can be injected into the target area as a cell suspension. Alternatively, the differentiated DA cells can be embedded in a support matrix or scaffold when contained in such a delivery device. In some embodiments, the scaffold is biodegradable. In other embodiments, the scaffold is not biodegradable. The scaffold can comprise natural or synthetic (artificial) materials.

The delivery of the DA neurons can be achieved by using a suitable vehicle such as, but not limited to, liposomes, microparticles, or microcapsules. In other embodiments, the differentiated DA neurons are administered in a pharmaceutical composition comprising an isotonic excipient. The pharmaceutical composition is prepared under conditions that are sufficiently sterile for human administration. In some embodiments, the DA neurons differentiated from HIP cells are supplied in the form of a pharmaceutical composition. General principles of therapeutic formulations of cell compositions are found in Cell Therapy: Stem Cell Transplantation, Gene Therapy, and Cellular Immunotherapy, G. Morstyn & W. Sheridan eds, Cambridge University Press, 1996, and Hematopoietic Stem Cell Therapy, E. Ball, J. Lister & P. Law, Churchill Livingstone, 2000, the disclosures are incorporated herein by reference.

Useful descriptions of neurons derived from stem cells and methods of making thereof can be found, for example, in Kirkeby et al., Cell Rep, 2012, 1:703-714; Kriks et al., Nature, 2011, 480:547-551: Wang et al., Stem Cell Reports, 2018, 11(1):171-182; Lorenz Studer, “Chapter 8—Strategies for Bringing Stem Cell-Derived Dopamine Neurons to the clinic-The NYSTEM Trial” in Progress in Brain Research, 2017, volume 230, pg. 191-212; Liu et al., Nat Protoc, 2013, 8:1670-1679; Upadhya et al., Curr Protoc Stem Cell Biol, 38, 2D.7.1-2D.7.47; US Publication Appl. No. 20160115448, and U.S. Pat. Nos. 8,252,586; 8,273,570; 9,487,752 and U.S. Pat. No. 10,093,897, the contents are incorporated herein by reference in their entirety.

In addition to DA neurons, other neuronal cells, precursors, and progenitors thereof can be differentiated from the HIP cells outlined herein by culturing the cells in medium comprising one or more factors or additive. Non-limiting examples of factors and additives include GDNF, BDNF, GM-CSF, B27, basic FGF, basic EGF, NGF, CNTF, SMAD inhibitor, Wnt antagonist, SHH signaling activator, and any combination thereof. In some embodiments, the SMAD inhibitor is selected from the group consisting of SB431542, LDN-193189, Noggin PD169316, SB203580, LY364947, A77-01, A-83-01, BMP4, GW788388, GW6604, SB-505124, lerdelimumab, metelimumab, GC-1008, AP-12009, AP-11014, LY550410, LY580276, LY364947, LY2109761, SB-505124, E-616452 (RepSox ALK inhibitor), SD-208, SM16, NPC-30345, K 26894, SB-203580, SD-093, activin-M108A, P144, soluble TBR2-Fc, DMH-1, dorsomorphin dihydrochloride and derivatives thereof. In some embodiments, the Wnt antagonist is selected from the group consisting of XAV939, DKK1, DKK-2, DKK-3, DKK-4, SFRP-1, SFRP-2, SFRP-3, SFRP-4, SFRP-5, WIF-1, Soggy, IWP-2, IWR1, ICG-001, KY0211, Wnt-059, LGK974, IWP-L6 and derivatives thereof. In some embodiments, the SHH signaling activator is selected from the group consisting of Smoothened agonist (SAG), SAG analog, SHH, C25-SHH, C24-SHH, purmorphamine, Hg-Ag and/or derivatives thereof.

In some embodiments, the neurons express one or more of the markers selected from the group consisting of glutamate ionotropic receptor NMDA type subunit 1 GRIN1, glutamate decarboxylase 1 GAD1, gamma-aminobutyric acid GABA, tyrosine hydroxylase TH, LIM homeobox transcription factor 1-alpha LMX1A. Forkhead box protein O1 FOXO1, Forkhead box protein A2 FOXA2, Forkhead box protein O4 FOXO4, FOXG1, 2′,3′-cyclic-nucleotide 3′-phosphodiesterase CNP, myelin basic protein MBP, tubulin beta chain 3 TUB3, tubulin beta chain 3 NEUN, solute carrier family 1 member 6 SLC1A6, SST, PV, calbindin, RAX, LHX6, LHX8, DLX1, DLX2, DLX5, DLX6, SOX6, MAFB, NPAS1, ASCL1, SIX6, OLIG2, NKX2.1, NKX2.2, NKX6.2, VGLUT1, MAP2, CTIP2, SATB2, TBR1, DLX2, ASCL1, ChAT, NGFI-B, c-fos, CRF, RAX, POMC, hypocretin, NADPH, NGF, Ach, VAChT, PAX6, EMX2p75, CORIN, TUJ1, NURR1, and/or any combination thereof.

### 3) Glial Cells

Glial cells to be used in a cell therapy product may be profiled for donor capability at any stage of the manufacturing process of the cell therapy product.

Glial cells used in a cell therapy product may be primary glial cells. Methods for profiling a population of cells for donor capability as described anywhere herein may be performed on primary (e.g., genome-edited) glial cells.

As described elsewhere herein, glial cells used in a cell therapy product may be pluripotent stem cell (iPSC)-derived glial cells. Methods for profiling a population of cells for donor capability as described anywhere herein may also be performed on stem cells capable of differentiating to form glial cells. Methods for profiling a population of cells for donor capability as described anywhere herein may also be performed on stem cell derived (e.g., genome-edited) glial cells.

Relevant information concerning glial cells as referred to in the context of the present disclosure is known in the art, including certain information regarding desired features of glial cells when used for cell therapy. It will be understood that embodiments concerning glial cells described herein may be readily and appropriately combined with embodiments describing HIP cells (e.g., exhibiting reduced expression of one or more molecules of the MHC class I and/or MHC class II molecules and increased expression of at least one tolerogenic factor), as well as embodiments describing safety switches, and other modified/gene edited cells as described herein. Glial cells to be used in a cell therapy product may be profiled for donor capability at any stage of the editing process during manufacturing of the cell therapy product.

In some embodiments, the neural cells described include glial cells such as, but not limited to, microglia, astrocytes, oligodendrocytes, ependymal cells and Schwann cells, glial precursors, and glial progenitors thereof are produced by differentiating pluripotent stem cells into therapeutically effective glial cells and the like. Differentiation of hypoimmunogenic pluripotent stem cells produces hypoimmunogenic neural cells, such as hypoimmunogenic glial cells.

In some embodiments, glial cells, precursors, and progenitors thereof generated by culturing pluripotent stem cells in medium comprising one or more agents selected from the group consisting of retinoic acid, IL-34, M-CSF, FLT3 ligand, GM-CSF, CCL2, a TGFbeta inhibitor, a BMP signaling inhibitor, a SHH signaling activator, FGF, platelet derived growth factor PDGF, PDGFR-alpha, HGF, IGF1, noggin, SHH, dorsomorphin, noggin, and any combination thereof. In certain instances, the BMP signaling inhibitor is LDN193189, SB431542, or a combination thereof. In some embodiments, the glial cells express NKX2.2. PAX6, SOX10, brain derived neurotrophic factor BDNF, neutrotrophin-3 NT-3. NT-4, EGF, ciliary neurotrophic factor CNTF, nerve growth factor NGF, FGF8, EGFR, OLIG1, OLIG2, myelin basic protein MBP, GAP-43, LNGFR, nestin, GFAP, CD11b, CD11c, CX3CR1, P2RY12, IBA-1, TMEM119, CD45, and any combination thereof. Exemplary differentiation medium can include any specific factors and/or small molecules that may facilitate or enable the generation of a glial cell type as recognized by those skilled in the art.

To determine if the cells generated according to the in vitro differentiation protocol display glial cell characteristics and features, the cells can be transplanted into an animal model. In some embodiments, the glial cells are injected into an immunocompromised mouse, e.g., an immunocompromised shiverer mouse. The glial cells are administered to the brain of the mouse and after a pre-selected amount of time the engrafted cells are evaluated. In some instances, the engrafted cells in the brain are visualized by using immunostaining and imaging methods. In some embodiments, it is determined that the glial cells express known glial cell biomarkers.

Useful methods for generating glial cells, precursors, and progenitors thereof from stem cells are found, for example, in U.S. Pat. Nos. 7,579,188; 7,595,194; 8,263,402; US8,206,699; US8,252,586; U.S. Pat. No. 9,193,951: US9,862,925; US8,227,247; U.S. Pat. No. 9,709,553; US2018/0187148; US2017/0198255; US2017/0183627; US2017/0182097: US2017/253856; US2018/0236004; WO2017/172976; and WO2018/093681. Methods for differentiating pluripotent stem cells are described in, e.g., Kikuchi et al., Nature, 2017. 548, 592-596; Kriks et al., Nature, 2011, 547-551; Doi et al., Stem Cell Reports, 2014, 2, 337-50; Perrier et al., Proc Natl Acad Sci USA, 2004, 101, 12543-12548; Chambers et al., Nat Biotechnol, 2009, 27, 275-280; and Kirkeby et al., Cell Reports, 2012, 1, 703-714.

The efficacy of neural cell transplants for spinal cord injury can be assessed in, for example, a rat model for acutely injured spinal cord, as described by McDonald, et al., Nat. Med., 1999. 5:1410) and Kim, et al., Nature, 2002, 418:50. For instance, successful transplants may show transplant-derived cells present in the lesion 2-5 weeks later, differentiated into astrocytes, oligodendrocytes, and/or neurons, and migrating along the spinal cord from the lesioned end, and an improvement in gait, coordination, and weight-bearing. Specific animal models are selected based on the neural cell type and neurological disease or condition to be treated.

The neural cells can be administered in a manner that permits them to engraft to the intended tissue site and reconstitute or regenerate the functionally deficient area. For instance, neural cells can be transplanted directly into parenchymal or intrathecal sites of the central nervous system, according to the disease being treated. In some embodiments, any of the neural cells described herein including cerebral endothelial cells, neurons, dopaminergic neurons, ependymal cells, astrocytes, microglial cells, oligodendrocytes, and Schwann cells are injected into a patient by way of intravenous, intraspinal, intracerebroventricular, intrathecal, intra-arterial, intramuscular, intraperitoneal, subcutaneous, intramuscular, intra-abdominal, intraocular, retrobulbar and combinations thereof. In some embodiments, the cells are injected or deposited in the form of a bolus injection or continuous infusion. In certain embodiments, the neural cells are administered by injection into the brain, apposite the brain, and combinations thereof. The injection can be made, for example, through a burr hole made in the subject's skull. Suitable sites for administration of the neural cell to the brain include, but are not limited to, the cerebral ventricle, lateral ventricles, cisterna magna, putamen, nucleus basalis, hippocampus cortex, striatum, caudate regions of the brain and combinations thereof.

Additional descriptions of neural cells including dopaminergic neurons for use in the present technology are found in WO2020/018615, the disclosure of which is herein incorporated by reference in its entirety.

Cells disclosed herein may be glial cells, for example a population of glial cells. It will be understood that any reference to “a cell” e.g. “a glial cell” below also applies to “a population of cells” e.g. “a population of glial cells” as described in the present application. Relevant information concerning glial cells as referred to in the context of the present disclosure may be found from WO2021195426, the contents of which are herein incorporated by reference. It will be understood that embodiments concerning glial cells described herein may be readily and appropriately combined with embodiments describing safety switches, as well as embodiments describing HIP cells, CAR cells and other modified/gene edited cells as described herein.

### Differentiation Methods to Generate Neural Cells

Different neural cell types may be differentiated from pluripotent stem cells including induced pluripotent stem cells. The neural cell types are useful for subsequent transplantation or engraftment into subjects in need thereof. As will be appreciated by those in the art, the methods for differentiation depend on the desired cell type using known techniques. Examples of such methods are described in WO2021195426 the contents of which are incorporated herein by reference in their entirety.

In some embodiments, differentiation of pluripotent stem cells such as induced pluripotent stem cells is performed by exposing or contacting cells to specific factors which are known to produce a specific cell lineage(s), so as to target their differentiation to a specific, desired lineage and/or cell type of interest. In some embodiments, terminally differentiated neural cells display specialized phenotypic characteristics or features. In some embodiments, the pluripotent stem cells are differentiated into neuroectodermal cells, neuronal cells, neuroendocrine cells, dopaminergic neurons, cholinergic neurons, serotonergic neurons, glutamatergic neurons, GABAergic neurons, adrenergic, noradrenergic neurons, sympathetic neurons, parasympathetic neurons, sympathetic peripheral neurons, glial cells, progenitors thereof, or precursors thereof. In some embodiments, the glial cells include microglial (e.g., amoeboid, ramified, activated phagocytic, and activated non-phagocytic) cells or macroglial cells (central nervous system cells: astrocytes, oligodendrocytes, ependymal cells, and radial glia; and peripheral nervous system cells: Schwann cells and satellite cells), precursors thereof, and progenitors of any of the preceding cells.

In some embodiments, the neural cells described include glial cells such as, but not limited to, microglia, astrocytes, oligodendrocytes, ependymal cells and Schwann cells, glial precursors, and glial progenitors thereof are produced by differentiating pluripotent stem cells into therapeutically effective glial cells and the like.

Neural cells may be derived from stem cells and then used to treat neurological disorders and conditions. Neural cell types may be produced from other non-neural cell types, including, but not limited to, pluripotent stem cells, induced pluripotent stem cells (iPSCs), and the like. Methods of obtaining such cells are described, for example, in WO2021195426 the contents of which are incorporated herein by reference in their entirety.

Some desired features of glial cells when used for cell therapy are described herein.

In some embodiments, the glial cells express NKX2.2, PAX6, SOX1 0, brain derived neurotrophic factor BDNF, neutrotrophin-3 NT-3, NT-4, epidermal growth factor EGF, ciliary neurotrophic factor CNTF, nerve growth factor NGF, FGF8, EGFR, OLIG1, OLIG2, myelin basic protein MBP, GAP-43, LNGFR, nestin, GFAP, CD11b, CD11c, CD105, CX3CR1, P2RY12, IBA-1, TMEM119, CD45, and any combination thereof. Any of these exemplary markers can be used to characterize glial cells described herein. To monitor glial cell differentiation as well as to assess the phenotype of a glial cell, the expression of any number of molecular and genetic markers specific to glial cells and progenitors thereof can be evaluated. For example, the presence of genetic markers can be determined by various methods known to those skilled in the art. Expression of molecular markers can be determined by quantifying methods such as, but not limited to, qPCR-based assays. RNA-seq assays, proteomic assays, immunoassays, immunocytochemistry assays, immunoblotting assays, and the like.

In some embodiments, the glial cells including oligodendrocytes, astrocytes, and progenitors thereof express one or more of the markers selected from A2B5, CD9, CD133, CD140a, FOXG1, GalC, GD3, GFAP, nestin, NG2, MBP, Musashi, 04, Olig1, Olig2, PDGFaR, S1OOP, glutamine synthetase, connexin 43, vimentin, BLBP, GLAST, and the like. In some embodiments, the glial cells including oligodendrocytes, astrocytes, and progenitors thereof do not express one or more of the markers selected from PSA-NCAM, CD9, CD11, CD32, CD36, CD105, CD140a, nestin, PDGFaR, and the like. In some embodiments, the glial cells are selected or purified using a positive selection strategy, a negative selection strategy, or both.

In some embodiments, glial cells are characterized according to morphology as determined by immunocytochemistry and immunohistochemistry. In some embodiments, glial cells are assessed according to functional characterization assays such as, but not limited to, a neuronal co-culture assay, stimulation assay with lipopolysaccharides (LPS), in vitro myelination assay, ATP influx with calcium wave oscillation assay, and the like.

In some embodiments, to determine that the glial cells display cell-specific characteristics and features, the cells can be transplanted into an animal model. In some embodiments, the glial cells are injected into an immunocompromised mouse, e.g., an immunocompromised shiverer mouse. The glial cells are administered to the brain of the mouse and after a pre-selected amount of time, the engrafted cells are evaluated. In some embodiments, the engrafted cells in the brain are visualized using immunostaining and imaging methods. In some embodiments, expression of known glial cell biomarkers can be determined in the engrafted cells.

Protocols for generating different types of neural cells are described in PCT Application No. WO2010144696, U.S. Pat. Nos. 9.057,053; 9,376,664; and 10,233,422. Additional descriptions of methods for differentiating hypoimmunogenic pluripotent cells can be found, for example, in Deuse et al., Nature Biotechnology, 2019, 37, 252-258 and Han et al., Proc Natl Acad Sci USA, 2019, 116(21), 10441-10446.

In some embodiments, glial cells, precursors, and progenitors thereof are generated by culturing pluripotent stem cells in medium comprising one or more agents selected from the group consisting ofretinoic acid, IL-34, M-CSF, FLT3 ligand, GM-CSF, CCL2, a TGFbeta inhibitor, a BMP signaling inhibitor, a SHH signaling activator, FGF, platelet derived growth factor PDGF, PDGFR-alpha, HGF, IGF-1, noggin, sonic hedgehog (SHH), dorsomorphin, noggin, and any combination thereof. In certain instances, the BMP signaling inhibitor is LDN193189, SB431542, or a combination thereof. Exemplary differentiation medium can include any specific factors and/or small molecules that may facilitate or enable the generation of a glial cell type as recognized by those skilled in the art.

In some embodiments, differentiation of pluripotent stem cells is performed by exposing or contacting cells to specific factors which are known to produce a glial cell such as a microglial cell (such as a amoeboid, ramified, activated phagocytic, and activated non-phagocytic cell), a macroglial cell (such as a astrocyte, oligodendrocyte, ependymal cell, radial glia, Schwann cell and satellite cell, a precursor thereof, and a progenitor thereof. Useful methods for generating glial cells, precursors, and progenitors thereof from stem cells are found, for example, in U.S. Pat. Nos. 7,579,188; 7,595,194; 8,263,402: 8,206,699; 8,227,247; 8,252,586; 9,193,951; 9,709,553; and 9,862,925; and US Puhl. Application Nos. 2018/0187148: 2017/0198255; 2017/0183627; 2017/0182097; 2017/253856; 2018/0236004; and PCT Puhl. Application Nos. WO2017/172976 and WO2018/093681.

The glial cells described herein may be used to treat or prevent a disease in a subject.

The glial cells described herein can be used to treat various neurological disorders and conditions.

In some embodiments, the glial cells described herein are administered to a subject to ameliorate or treat stroke. In some embodiments, the glial cells are administered to a subject who has experienced a stroke.

In some embodiments, the glial cells described herein are administered to a subject to alleviate a symptom or effect of amyotrophic lateral sclerosis (ALS). In some embodiments, the glial cells are administered to a subject with ALS.

In some embodiments, the glial cells described herein are administered to a subject to alleviate a symptom or effect of cerebral hemorrhage. In some embodiments, the glial cells are administered to a subject who has experienced a cerebral hemorrhage.

In some embodiments, the glial cells described herein are administered to a subject to alleviate a symptom or effect of Parkinson's disease. In some embodiments, the glial cells are administered to a patient with Parkinson's disease.

In some embodiments, the glial cells described herein are administered to a subject to alleviate a symptom or effect of an epileptic seizure. In some embodiments, the glial cells are administered to a patient who has experienced an epileptic seizure.

In some embodiments, the glial cells described herein are administered to a subject to alleviate a symptom or effect of a spinal cord injury. In some embodiments, the glial cells are administered to a patient who has experienced a spinal cord injury.

In some embodiments, the glial cells described herein are administered to a subject to alleviate a symptom or effect of Pelizaeus-Merzbacher Disease. In some embodiments, the glial cells are administered to a subject with Pelizaeus-Merzbacher Disease.

In some embodiments, the glial cells described herein are administered to a subject to alleviate a symptom or effect of progressive multiple sclerosis. In some embodiments, the glial cells are administered to a subject with progressive multiple sclerosis.

In some embodiments, the glial cells described herein are administered to a subject to alleviate a symptom or effect of Huntington's Disease. In some embodiments, the glial cells are administered to a subject Huntington's Disease.

### I. Macrophages

Macrophages to be used in a cell therapy product may be profiled for donor capability at any stage of the manufacturing process of the cell therapy product.

Macrophages used in a cell therapy product may be primary macrophages. Methods for profiling a population of cells for donor capability as described anywhere herein may be performed on primary (e.g., genome-edited) macrophages.

Macrophages used in a cell therapy product may be pluripotent stem cell (iPSC)-derived macrophages. Methods for profiling a population of cells for donor capability as described anywhere herein may also be performed on stem cells capable of differentiating to form macrophages. Methods for profiling a population of cells for donor capability as described anywhere herein may also be performed on stem cell derived (e.g., genome-edited) macrophages.

Relevant information concerning macrophages as referred to in the context of the present disclosure is known in the art, including information regarding desired features of macrophages when used for cell therapy and, for example, may be found from WO2017019848, the contents of which are herein incorporated by reference. It will be understood that embodiments concerning macrophages described herein may be readily and appropriately combined with embodiments describing HIP cells (e.g., exhibiting reduced expression of one or more molecules of the MHC class I and/or MHC class II molecules and increased expression of at least one tolerogenic factor), as well as embodiments describing safety switches, and other modified/gene (e.g. CAR transgene) edited cells as described herein. Macrophages to be used in a cell therapy product may be profiled for donor capability at any stage of the editing process during manufacturing of the cell therapy product.

The macrophages described herein may be used to treat or prevent a disease in a subject.

The cells used in embodiment may be macrophages or other phagocytic cells, for example a population of macrophages. It will be understood that any reference to “a cell” e.g. “a macrophage” below also applies to “a population of cells” e.g. “a population of macrophages” as described in the present application.

Phagocytic cells, such as monocytes, macrophages and/or dendritic cells, may be obtained from a subject (for example, as described in WO2017019848A1 the contents of which are incorporated herein by reference in their entirety). Non-limiting examples of subjects include humans, dogs, cats, mice, rats, and transgenic species thereof. Preferably, the subject is a human. The cells can be obtained from a number of sources, including peripheral blood mononuclear cells, bone marrow, lymph node tissue, spleen tissue, umbilical cord, and tumors. In some embodiments, any number of monocyte, macrophage, dendritic cell or progenitor cell lines available in the art, may be used. In some embodiments, the cells can be obtained from a unit of blood collected from a subject using any number of techniques known to the skilled artisan, such as Ficoll separation. In some embodiments, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In some embodiments, cells are isolated from peripheral blood by lysing the red blood cells and depleting the lymphocytes and red blood cells, for example, by centrifugation through a PERCOLL™ gradient. Alternatively, cells can be isolated from umbilical cord. In any event, a specific subpopulation of the monocytes, macrophages and/or dendritic cells can be further isolated by positive or negative selection techniques.

In some embodiments, a population of cells comprises the monocytes, macrophages, or dendritic cells. Examples of a population of cells include, but are not limited to, peripheral blood mononuclear cells, cord blood cells, a purified population of monocytes, macrophages, or dendritic cells, and a cell line. In some embodiments, peripheral blood mononuclear cells comprise the population of monocytes, macrophages, or dendritic cells. In some embodiments, purified cells comprise the population of monocytes, macrophages, or dendritic cells.

Some desired features of macrophages when used for cell therapy are described herein.

In some embodiments, the cells have upregulated M1 markers and downregulated M2 markers. For example, at least one M1 marker, such as HLA DR, CD86, CD80, and PDL1, is upregulated in the phagocytic cell. In another example, at least one M2 marker, such as CD206, CD163, is downregulated in the phagocytic cell (for example, as described in WO2017019848A1 the contents of which are incorporated herein by reference in their entirety). In some embodiments, the cell has at least one upregulated M1 marker and at least one downregulated M2 marker.

In some embodiments, targeted effector activity in the phagocytic cell is enhanced by inhibition of either CD47 or SIRPa activity. CD47 and/or SIRPa activity may be inhibited by treating the phagocytic cell with an anti-CD47 or anti-SIRPa antibody. Alternatively, CD47 or SIRPa activity may be inhibited by any method known to those skilled in the art.

Macrophages may be obtained according to the following exemplary methods, as described in WO2017019848A1 (the contents of which are incorporated herein by reference in their entirety).

Cell Culture as described in WO2017019848A1: THP1, K562, SKOV3, SKBR3, HDLM2, MD468, and all cell lines may be cultured in RPMI 1640 supplemented with 10% fetal bovine serum and penicillin/streptomycin at 37C in 5%/C02. A THP1 mRFP+ subline (Wt) may be generated by lentiviral transduction and FACS purification of mRFP+ cell lines. The THP1 mRFP+ subline may be used to generate THP1 mRFP+ CAR19z+ (CAR19z; CARMA19z), THP1 mRFP+ CAR19Az+ (CAR19Az; CARMA19Az), THP1 mRFP+ MesoZ+ and THP1 mRFP+ CARHer2z+ (CARHer2z: CARMAHer2z) sublines. Monocyte differentiation may be induced by culturing cells for 48 hours with Ing/mL phorbol 12-myristate 13-acetate in culture media.

Primary Human Macrophages as described in WO2017019848A1: Primary human monocytes may be purified from normal donor apheresis product using Miltenyi CD14 MicroBeads (Miltenyi, 130-050-201). Monocytes may be cultured in X-Vivo media supplemented with 5% human AB serum or RPMI 1640 supplemented with 10% fetal bovine serum, with penicillin/streptomycin, glutamax, and 10 ng/mL recombinant human GM-CSF (PeproTech, 300-03) for 7 days in MACS GMP Cell Differentiation Bags (Miltenyi, 170-076-400). Macrophages may be harvested on day 7 and cryopreserved in FBS+10% DMSO pending subsequent use.

Phagocytosis Assay as described in WO2017019848A1: Wt or CARMA mRFP+ THP1 sublines may be differentiated for 48 hours with Ing/mL phorbol 12-myristate 13-acetate. GFP+ antigen bearing tumor sublines, i.e. K562 CD19+GFP+ cells, may be added to the differentiated THP1 macrophages at a 1:1 ratio following PMA washout. Macrophages may be co-cultured with target tumor cells for 4 hours, and phagocytosis was quantified by fluorescent microscopy using the EVOS FL Auto Cell Imaging System. An average of three fields of view may be considered as n, and all conditions may be quantified in triplicates. FACS based phagocytosis may be analyzed on a BD LSR-Fortessa. FlowJo (Treestar, Inc.) may be used to analyze flow cytometric data. Live, singlets gated mRFP/GFP double positive events may be considered phagocytosis. CD47/SIRPa axis blockade may be performed via addition of blocking monoclonal antibodies at the initiation of co-culture at indicated concentrations (mouse anti-human CD47 clone B6H12, eBioscience #14-0479-82; mouse anti-human CD47 clone 2D3 as negative control, eBioscience #14-0478-82; mouse anti-human SIRPa clone SE5A5, BioLegend #323802). TLR co-stimulation may be performed by adding TLR1-9 agonists (Human TLR 1-9 agonist kit; Invivogen #tlr1-kitlhw) at the time of co-culture.

In Vitro Killing Assay as described in WO2017019848A1: Wt or CAR bearing macrophages may be co-cultured with antigen-bearing or control click-beetle green luciferase (CBG)/green fluorescent protein (GFP) positive target tumor cells at varying effector to target ratios (starting at 30: 1 and decreasing in three-fold dilutors). Bioluminescent imaging may be utilized to determine tumor burden, using the IVIS Spectrum Imaging System (Perkin Elmer). Percent specific lysis was calculated as follows: % Specific Lysis=((Treated well−Tumor alone well)/(Maximal killing−tumor alone well)*100)

Time-Lapse Microscopy as described in WO2017019848A1: Fluorescent time-lapse video microscopy of CAR mediated phagocytosis may be performed using the EVOS FL Auto Cell Imaging System. Images may be captured every 40 seconds for 18 hours. Image analysis was performed with FIJI imaging software.

Lentiviral production and transfection as described in WO2017019848A1: Chimeric antigen receptor constructs may be de novo synthesized by GeneArt (Life Technologies) and cloned into a lentiviral vector as previously described. Concentrated lentivirus may be generated using HEK293T cells as previously described.

Adenoviral production and transfection as described in WO2017019848A1: Ad5f35 chimeric adenoviral vectors encoding GFP, CAR, or no transgene under a CMV promoter may be produced and titrated as per standard molecular biology procedure. Primary human macrophages may be transduced with varying multiplicities of infection and serially imaged for GFP expression and viability using the EVOS FL Auto Cell Imaging System. CAR expression may be assessed by FACS analysis of surface CAR expression using His-tagged antigen and anti-His-APC secondary antibody (R&D Biosystems Clone ADl 0.1.10).

Flow Cytometry as described in WO2017019848A1: FACS may be performed on a BD LSR Fortessa. Surface CAR expression was detected with biotinylated protein L (GenScript M00097) and streptavidin APC (BioLegend, #405207) or His-tagged antigen and anti-His-APC secondary antibody (R&D Biosystems Clone AD1 0.1.10). Fc receptors may be blocked with Human Trustain FcX (BioLegend, #422301) prior to staining. CD47 expression may be determined using mouse anti- human CD47 APC (eBioscience #17-0479-41) with mouse IgG1 kappa APC isotype control for background determination. Calreticulin expression may be determined with mouse anti-calreticulin PE clone FMC75 (Abeam #ab83220). All flow results may be gated on live (Live/Dead Aqua Fixable Dead Cell Stain. Life Technologies L34957) single cells.

Imagestream Cytometry as described in WO2017019848A1: FACS with single cell fluorescent imaging may be performed on an ImageStream Mark II Imaging Flow Cytometer (EMD Millipore). Briefly, mRFP+ or Dil stained macrophages (CAR or control) may be co-cultured with GFP+ tumor cells for 4 hours, prior to fixation and ImageStream data acquisition. Data may be analyzed using ImageStream software (EMD Millipore).

RNA Electroporation as described in WO2017019848A1: CAR constructs may be cloned into in vitro transcription plasmids under the control of a T7 promoter using standard molecular biology techniques. CAR mRNA may be in vitro transcribed using an mMessage mMachine T7 Ultra In Vitro Transcription Kit (Thermo Fisher), purified using RNEasy RNA Purification Kit (Qiagen), and electroporated into human macrophages using a BTX ECM850 electroporator (BTX Harvard Apparatus). CAR expression may be assayed at varying time points post-electroporation using FACS analysis.

TLR/Dectin-1 Priming as described in WO2017019848A1: TLR or Dectin-1 priming in Wt or CAR macrophages prior to in vitro phagocytosis or killing assays may be performed by pre-incubating the cells with recommended doses of either TLR 1-9 agonists (Human TLR1-9 Agonist Kit, Invivogen) or beta-glucan (MP Biomedicals, LLC), respectively, for 30 minutes prior to co-culture. In vitro function of Wt or CAR macrophages may be compared between unprimed and primed conditions.

Macrophage/Monocyte Phenotype as described in WO2017019848A1: The following surface markers may be assessed as part of a macrophage/monocyte immunophenotype FACS panel, for M1/M2 distinction: CD80. CD86, CD163, CD206, CD11B, HLA-DR, HLA-A/B/C, PDL1, and PDL2 (BioLegend). TruStain FcX may be used for Fc receptor blockade prior to immunostaining. Macrophages/monocytes may be exposed to activating conditions, i.e. Ad5f35 transduction for 48 hours, or not, prior to phenotype assessment.

Seahorse Assay as described in WO2017019848A1: Metabolic phenotype and oxygen consumption of macrophages may be determined using the Seahorse assay (Seahorse XF, Agilent). Control or CAR macrophages may be exposed to media control or immunosuppressive cytokines for 24 hours prior to analysis. Cells may be treated with oligomycin, FCCP, and rotenone sequentially throughout the Seahorse assay. The assay was performed with 6 replicates per condition.

In Vivo Assays as described in WO2017019848A1: NOD-scid IL2Rg-null-IL3/GM/SF, NSG-SGM3 (NSGS) mice may be used used for human xenograft models. Mice engrafted with CBG-luciferase positive human SKOV3 ovarian cancer cells were either left untreated, or treated with untransduced, empty Ad5f35 transduced, or Ad5f35 CAR-HER2 transduced human macrophages at different doses. Serial bioluminescent imaging may be performed to monitor tumor burden (IVIS Spectrum, Perkin Elmer). Organs and tumor may be harvested upon sacrifice for FACS analysis. Overall survival may be monitored and compared using Kaplan-Meier analysis.

Macrophages described herein may be used to treat or prevent a disease in a subject.

In some embodiments, the disease is an autoimmune disease. The term “autoimmune disease” as used herein is defined as a disorder that results from an autoimmune response. An autoimmune disease is the result of an inappropriate and excessive response to a self-antigen. Examples of autoimmune diseases include but are not limited to, Addision's disease, alopecia areata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, mxedema, pernicious anemia, ulcerative colitis, among others.

Examples of autoimmune disease include but are not limited to, Acquired Immunodeficiency Syndrome (AIDS, which is a viral disease with an autoimmune component), alopecia areata, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigoid, cold agglutinin disease, crest syndrome, Crohn's disease, Degos' disease, dermatomvositis- juvenile, discoid lupus, essential mixed cryoglobulinemia, fibromvalgia-fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, Meniere's disease, mixed connective tissue disease, multiple sclerosis, myasthenia gravis, pernacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma (progressive systemic sclerosis (PSS), also known as systemic sclerosis (SS)), Sjogren's syndrome, stiff-man syndrome, systemic lupus erythematosus, Takayasu arteritis, temporal arteritis/giant cell arteritis, ulcerative colitis, uveitis, vitiligo and Wegener's granulomatosis.

The macrophages described herein can also be used to treat inflammatory disorders. Examples of inflammatory disorders include but are not limited to, chronic and acute inflammatory disorders. Examples of inflammatory disorders include Alzheimer's disease, asthma, atopic allergy, allergy, atherosclerosis, bronchial asthma, eczema, glomerulonephritis, graft vs. host disease, hemolytic anemias, osteoarthritis, sepsis, stroke, transplantation of tissue and organs, vasculitis, diabetic retinopathy and ventilator induced lung injury.

In some embodiments the disease is cancer. The macrophages described herein can be used to treat cancers. Cancers include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with cells disclosed herein include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas).

Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pineal oma, hemangioblastoma, acoustic neuroma, oligodendroglioma, menangioma, neuroblastoma, retinoblastoma and brain metastases).

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Further examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In some embodiments, the cancer is medullary thyroid carcinoma.

### J. B Cells

B cells to be used in a cell therapy product may be profiled for donor capability at any stage of the manufacturing process of the cell therapy product.

B cells used in a cell therapy product may be primary B cells. Methods for profiling a population of cells for donor capability as described anywhere herein may be performed on primary (e.g., genome-edited) B cells.

As described elsewhere herein, B cells used in a cell therapy product may be pluripotent stem cell (iPSC)-derived B cells. Methods for profiling a population of cells for donor capability as described any where herein may also be performed on stem cells capable of differentiating to form B cells. Methods for profiling a population of cells for donor capability as described anywhere herein may also be performed on stem cell derived (e.g., genome-edited) B cells.

Relevant information concerning B cells as referred to in the context of the present disclosure is known in the art, including certain information regarding desired features of B cells when used for cell therapy and, for example, may be found from US2019321403A1, the contents of which are herein incorporated by reference. It will be understood that embodiments concerning B cells described herein may be readily and appropriately combined with embodiments describing HIP cells (e.g., exhibiting reduced expression of one or more molecules of the MHC class I and/or MHC class II molecules and increased expression of at least one tolerogenic factor), as well as embodiments describing safety switches, and other modified/gene (e.g. CAR transgene) edited cells as described herein. B cells to be used in a cell therapy product may be profiled for donor capability at any stage of the editing process during manufacturing of the cell therapy product.

The B cells described herein may be used to treat or prevent a disease in a subject.

Cells disclosed herein may be B cells, for example a population of B cells. It will be understood that any reference to “a cell” e.g. “a B cell” below also applies to “a population of cells” e.g. “a population of B cells” as described in the present application.

Among the sub-types and subpopulations of B cells are precursor or immature B cells, naïve mature B cells, memory B cells, plasmablasts, and plasma cells. Precursor or immature B cells include HSCs, multipotent progenitor (MPP) cells, common lymphoid progenitor (CLP) cells, early pro-B cells, late pro-B cells, large pre-B cells, small pre-B cells, immature B cells, T1 B cells, and T2 B cells (for example, as described in US2019321403A1 the contents of which are incorporated herein by reference in their entirety).

Methods of obtaining B cells or a population of B cells are known in the art (for example, as described in US2019321403A1 the contents of which are incorporated herein by reference in their entirety) In Various techniques for in vitro maturation of HSCs into secreting B lymphocytes and plasma cells are known (see for example Luo, X. M., et al. (2009). Blood, 113(7), 1422-1431).

As described in US2019321403A1 for example, the starting population of cells used may be derived from a number of sources. The starting cell population may be derived from PBMCs or other blood samples, tonsils, bone marrow or other like preparations in which B cells are present. In some embodiments, the starting population of cells may include bulk (non-selected) B cells or a specific B cell subset, such as mature, immature, memory, naïve, or other B cell subset. In some embodiments, the starting cell population may comprise precursor cells capable of differentiating into B cells, such as hematopoietic stem cells (HSCs). With reference to the subject to be treated, the starting cells may be allogeneic and/or autologous. Among the methods include off-the-shelf methods. In some aspects, such as for off-the-shelf technologies, the starting cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Some desired features of B cells when used for cell therapy are described herein.

In some embodiments, one or more of the B cell populations is enriched for or depleted of cells that are positive for (marker+) or express high levels (marker high) of one or more particular markers, such as surface markers, or that are negative for (marker−) or express relatively low levels (marker low) of one or more markers. In some cases, such markers are those that are absent or expressed at relatively low levels on certain populations of B cells (such as naïve cells) but are present or expressed at relatively higher levels on certain other populations of B cells (such as non-naïve cells).

In some embodiments, the cells are (1) enriched for (i.e., positively selected for) cells that are positive for or express high levels of one or more of (such as all of) PAX5, BACH2, BCL-2, OBF1, OCT2, PU.1, SPIB, ETS1, and IRF8 and/or depleted of (e.g., negatively selected for) cells that are positive for or express high levels of one or more of (such as all of) IRF4, BLIMP1, and XBP1; and/or (2) enriched for (i.e., positively selected for) cells that are positive for or express high surface levels of one or more of (such as all of) CD19, CD20, CD21, CD22, CD23, and CD24 and/or depleted of (e.g., negatively selected for) cells that are positive for or express high surface levels of one or more of (such as all of) CD10, CD27, and CD38. In some embodiments, the cells are enriched for naïve mature B cells.

In some embodiments, the cells are (1) enriched for (i.e., positively selected for) cells that are positive for or express high levels of one or more of (such as all of) IRF4, BLIMP1, and XBP1 and/or depleted of (e.g., negatively selected for) cells that are positive for or express high levels of one or more of(such as all of) PAX5. BACH2, BCL-2, OBF1, OCT2, PU.1, SPIB, ETS1, and IRF8; and/or (2) enriched for (i.e., positively selected for) cells that are positive for or express high surface levels of one or more of (such as all of) CD19, CD38, CD27, CD269, and MHCII and/or depleted of (e.g., negatively selected for) cells that are positive for or express high surface levels of CD20 and/or CD138. In some embodiments, the cells are enriched for plasmablasts.

In some embodiments, the cells are (1) enriched for (i.e., positively selected for) cells that are positive for or express high levels of one or more of (such as all of) IRF4, BLIMP1, and XBP1 and/or depleted of (e.g., negatively selected for) cells that are positive for or express high levels of one or more of (such as all of) PAX5, BACH2, BCL-2, OBF1, OCT2, PU.1, SPIB, ETS1, and IRF8; and/or (2) enriched for (i.e., positively selected for) cells that are positive for or express high surface levels of one or more of (such as all of) CXCR4, CD27, CD38, CD138, and CD269 and/or depleted of (e.g., negatively selected for) cells that are positive for or express high surface levels of one or more of (such as all of) CD19, CD20, and MHCII. In some embodiments, the cells are enriched for plasma cells.

In some embodiments, the cells are (1) enriched for (i.e., positively selected for) cells that are positive for or express high levels of one or more of (such as all of) PAX5, BACH2, BCL-2, OBF1, OCT2, PU.1, SPIB, ETS1, and IRF8 and/or depleted of (e.g., negatively selected for) cells that are positive for or express high levels of one or more of(such as all of) IRF4, BLIMPL and XBP1; and/or (2) enriched for (i.e., positively selected for) cells that are positive for or express high surface levels of one or more of (such as all of) CD19, CD20, CD40, CXCR4, CXCR5, and CXCR7 and/or depleted of (e.g., negatively selected for) cells that are positive for or express high surface levels of CD23 and/or CD38. In some embodiments, the cells are enriched for memory B cells.

Once the cells are administered to the subject (e.g., human), the biological activity of the engineered B cell populations in some aspects is measured by any of a number of known methods. In some embodiments, the biological activity of the cells can be measured by assaying for expression and/or secretion of the exogenous protein, such as therapeutic protein. In some embodiments, the biological activity of the cells also can be measured by assaying expression and/or secretion of certain cytokines, such as IFNγ, IL-2, IL-4, IL-6, IL-12 and TNFα. In some embodiments the biological activity is measured by assessing clinical outcome, such as reduction in tumor burden or load. In some embodiments, toxic outcomes, persistence and/or expansion of the cells, and/or presence or absence of a host immune response, are assessed.

In some embodiments, the methods comprise inducing the engineered B cell to increase production and/or secretion of the exogenous protein. In some embodiments, the inducing comprises administering to the subject an agent that binds to the ligand binding domain of an endogenous B cell receptor expressed in the engineered B cell. In some embodiments, the agent is a vaccine recognized by an endogenous B cell receptor, such as any as described. In some embodiments, the inducing comprises administering to the subject an agent that binds to the ligand binding domain of the driving receptor, such as a recombinant or chimeric receptor, expressed in the engineered B cell. In some embodiments, the binding of the ligand to the driving receptor of the engineered B cell induces the engineered B cell to differentiate into a plasmablast or a plasma cell. In some embodiments, the engineered B cell is a plasmablast or plasma cell. In some embodiments, the exogenous protein is under the control of an endogenous immunoglobulin promoter or a constitutively active promoter. In some embodiments, the exogenous protein is under the control of an inducible promoter, and the method further comprises administering to the subject an agent that activates the inducible promoter.

In some embodiments, the method results in a duration of action (the length of time that the particular method is effective) in a subject of at least about 1 month, at least 2 months, at least 6 months, at least a year, at least 2 years or more. In some embodiments, a single administration of the engineered B cell or composition to the subject results in an increased duration of action in the subject compared to the maximum tolerable duration of action (duration of action for the maximum tolerable dose of a therapeutic) resulting from a single direct administration of the exogenous protein to the subject. In some embodiments, the increase is at least 1.2-fold, 1.5-fold, 2-fold, 3-fold, 4-fold, or 5-fold.

The B cells described herein may be used to treat or prevent a disease in a subject.

Among the diseases, conditions, and disorders that may be treated using the B cells described herein are tumors, including solid tumors, hematologic malignancies, and melanomas, and infectious diseases, such as infection with a virus or other pathogen, e.g., HIV, HCV, HBV, CMV, and parasitic disease. In some embodiments, the disease or condition is a tumor, cancer, malignancy, neoplasm, or other proliferative disease. Such diseases include but are not limited to hematological (or hematogenous) cancers including leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, plasmacytoma, Waldenstrom's macroglobulinemia, heavy chain disease, myelodysplastic syndrome, hairy cell leukemia and myelodysplasia, and solid tumors including sarcomas and carcinomas, including adrenocortical carcinoma, cholangiocarcinoma, fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, stomach cancer, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, thyroid cancer (e.g., medullary thyroid carcinoma and papillary thyroid carcinoma), pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer (e.g., cervical carcinoma and pre-invasive cervical dysplasia), colorectal cancer, cancer of the anus, anal canal, or anorectum, vaginal cancer, cancer of the vulva (e.g., squamous cell carcinoma, intraepithelial carcinoma, adenocarcinoma, and fibrosarcoma), penile cancer, oropharyngeal cancer, esophageal cancer, head cancers (e.g., squamous cell carcinoma), neck cancers (e.g., squamous cell carcinoma), testicular cancer (e.g., seminoma, teratoma, embryonal carcinoma, teratocarcinoma, choriocarcinoma, sarcoma, Leydig cell tumor, fibroma, fibroadenoma, adenomatoid tumors, and lipoma), bladder carcinoma, kidney cancer, melanoma, cancer of the uterus (e.g., endometrial carcinoma), urothelial cancers (e.g., squamous cell carcinoma, transitional cell carcinoma, adenocarcinoma, ureter cancer, and urinary bladder cancer), and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

In some embodiments, the disease or condition is an infectious disease or condition, such as, but not limited to, viral, retroviral, bacterial, and protozoal infections. Such diseases include but are not limited to infection with a pathogen selected from among Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis. Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis. Chlamydophila pneumoniae, Chlamydophila psittaci, CJD prion, Clonorchis sinensis, Clostridium botulinum. Clostridium difficile. Clostridium perfringens, Clostridium perfringens, Clostridium spp, Clostridium tetani, Coccidioides spp, coronaviruses, Corynebacterium diphtheriae, Coxiella burnetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli O 157:H7, O111 and O104:H4, Fasciola hepatica and Fasciola gigantica, FPI prion, Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBoV), Human herpesvirus 6 (HHV-6) and Human herpesvirus 7 (HHV-7), Human metapneumovirus (hMPV), Human papillomavirus (HPV), Human parainfluenza viruses (HPIV), Human T cell leukemia virus 1 (HTLV-1). Japanese encephalitis virus, JC virus, Junin virus, Kaposi's Sarcoma associated herpesvirus (KSHV), Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus. Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans. Mycoplasma pneumoniae, Naegleria fowleri, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parvovirus B19, Pasteurella genus, Plasmodium genus, Pneumocystis jirovecii, Poliovirus, Rabies virus, Respiratory syncytial virus (RSV). Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus genus. Streptococcus agalactiae, Streptococcus pneumoniae, Streptococcus pyogenes, Strongyloides stercoralis. Taenia genus, Taenia solium, Tick-bome encephalitis virus (TBEV), Toxocara canis or Toxocara cati, Toxoplasma gondii, Treponema pallidum, Trichinella spiralis, Trichomonas vaginalis, Trichophyton spp. Trichuris trichiura, Trypanosoma brucei, Trypanosoma cruzi, Ureaplasma urealyticum, Varicella zoster virus (VZV), Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis.

In some embodiments, the disease or condition is HIV infection. In some embodiments, the HIV infection is HIV-1 or HIV-2 infection, including infection with any of the HIV groups, subtypes, or variants described herein. Exemplary HIV-1 groups include HIV-1 Group M, HIV-1 Group N. HIV-1 Group O, and HIV-1 Group P. Subtypes and recombinant forms thereof are known: exemplary subtypes include subtype A (including A1 and A2), subtype B, subtype C, and recombinant forms including CRF_AE. Exemplary HIV-2 groups include HIV-2 Group A, HIV-2 Group B, HIV-2 Group C, HIV-2 Group D, HIV-2 Group E, HIV-2 Group F, HIV-2 Group G, and HIV-2 Group H.

### K. +Hematopoietic Stem Cells (Hscs)

Methods for profiling a population of cells for donor capability as described anywhere herein may also be performed on hematopoietic stem cells (e.g., genome-edited).

Relevant information concerning hematopoietic stem cells as referred to in the context of the present disclosure is known in the art, including information regarding desired features of hematopoietic stem cells when used for cell therapy. It will be understood that embodiments concerning HSCs described herein may be readily and appropriately combined with embodiments describing HIP cells (e.g., exhibiting reduced expression of one or more molecules of the MHC class I and/or MHC class II molecules and increased expression of at least one tolerogenic factor), as well as embodiments describing safety switches, and other modified/gene edited cells as described herein. HSCs to be used in a cell therapy product may be profiled for donor capability at any stage of the manufacturing process of the cell therapy product.

The HSC described herein may be used to treat or prevent a disease in a subject.

In some embodiments, the engineered cell is a hematopoietic stem cell. In some cases, the hematopoietic stem cell is an immature cell that can develop into all types of blood cells, including white blood cells, red blood cells, and platelets. Hematopoietic stem cells (HSC) are found in the peripheral blood and the bone marrow. In some cases, the hematopoietic stem cell is isolated from the peripheral blood or bone marrow.

In some embodiments, an engineered HSC or population comprising engineered HSCs is administered to treat a hematopoietic disease or disorder. In some embodiments, the hematopoietic disease or disorder is myelodysplasia, aplastic anemia, Fanconi anemia, paroxysmal nocturnal hemoglobinuria, Sickle cell disease, Diamond Blackfan anemia, Schachman Diamond disorder, Kostmann's syndrome, chronic granulomatous disease, adrenoleukodystrophy, leukocyte adhesion deficiency, hemophilia, thalassemia, beta-thalassemia, leukaemia such as acute lymphocytic leukemia (ALL), acute myelogenous (myeloid) leukemia (AML), adult lymphoblastic leukaemia, chronic lymphocytic leukemia (CLL), B-cell chronic lymphocytic leukemia (B-CLL), chronic myeloid leukemia (CML), juvenile chronic myelogenous leukemia (CML), and juvenile myelomonocytic leukemia (JMML), severe combined immunodeficiency disease (SCID), X-linked severe combined immunodeficiency, Wiskott-Aldrich syndrome (WAS), adenosine-deaminase (ADA) deficiency, chronic granulomatous disease, Chediak-Higashi syndrome, Hodgkin's lymphoma, non-Hodgkin's lymphoma (NHL) or AIDS.

In some embodiments, an engineered HSC or population comprising engineered HSCs is administered to treat a cellular deficiency is associated with leukemia or myeloma, or to treat leukemia or myeloma.

In some embodiments, an engineered HSC or population comprising engineered HSCs is administered to treat a cellular deficiency associated with an autoimmune disease or condition or to treat an autoimmune disease or condition. In some embodiments, the autoimmune disease or condition is acute disseminated encephalomyelitis, acute hemorrhagic leukoencephalitis, Addison's disease, Agammaglobulinemia, Alopecia areata, amyotrophic lateral sclerosis, ankylosing spondylitis, antiphospholipid syndrome, antisynthetase syndrome, atopic allergy, autoimmune aplastic anemia, autoimmune cardiomyopathy, autoimmune enteropathy, autoimmune hemolytic anemia, autoimmune hepatitis, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticaria, autoimmune uveitis, Balo disease, Balo concentric sclerosis, Bechets syndrome, Berger's disease, Bickerstaffs encephalitis, Blau syndrome, bullous pemphigoid, cancer, Castleman's disease, celiac disease, chronic inflammatory demyelinating polyneuropathy, chronic recurrent multifocal osteomyelitis, Churg-Strauss syndrome, cicatricial pemphigoid, Cogan syndrome, cold agglutinin disease, complement component 2 deficiency, cranial arteritis, CREST syndrome, Crohn's disease, Cushing's syndrome, cutaneous leukocytoclastic angiitis, Dego's disease, Dercum's disease, dermatitis herpetiformis, dermatomyositis, diabetes mellitus type 1 diffuse cutaneous systemic sclerosis, Dressler's syndrome, discoid lupus erythematosus, eczema, enthesitis-related arthritis, eosinophilic fasciitis, eosinophilic gastroenteritis, epidermolysis bullosa acquisita, erythema nodosum, essential mixed cryoglobulinemia, Evan's syndrome, firodysplasia ossificans progressiva, fibrosing aveolitis, gastritis, gastrointestinal pemphigoid, giant cell arteritis, glomerulonephritis, goodpasture's syndrome. Grave's disease, Guillain-Barre syndrome (GBS), Hashimoto's encephalitis, Hashimoto's thyroiditis, hemolytic anaemia, Henoch-Schonlein purpura, herpes gestationis, hypogammaglobulinemia, idiopathic inflammatory demyelinating disease, idiopathic pulmonary fibrosis, idiopathic thrombocytopenic purpura, IgA nephropathy, inclusion body myositis, inflammatory demyelinating polyneuropathy, interstitial cystitis, juvenile idiopathic arthritis, juvenile rheumatoid arthritis, Kawasaki's disease, Lambert-Eaton myasthenic syndrome, leukocytoclastic vasculitis, lichen planus, lichen sclerosus, linear IgA disease (LAD), Lou Gehrig's disease, lupoid hepatitis, lupus erythematosus, Majeed syndrome, Meniere's disease, microscopic polyangiitis, Miller-Fisher syndrome, mixed connective tissue disease, morphea, Mucha-Habermann disease, multiple sclerosis, myasthenia gravis, myositis, neuropyelitis optica, neuromyotonia, ocular cicatricial pemphigoid, opsoclonus myoclonus syndrome, ord thyroiditis, palindromic rheumatism, paraneoplastic cerebellar degeneration, paroxysmal nocturnal hemoglobinuria (PNH), Parry Romberg syndrome, Parsonnage-Tumer syndrome, pars planitis, pemphigus, pemphigus vulgaris, permicious anemia, perivenous encephalomyelitis, POEMS syndrome, polyarteritis nodosa, polymyalgia rheumatica, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restless leg syndrome, retroperitoneal fibrosis, rheumatoid arthritis, rheumatoid fever, sarcoidosis, Schmidt syndrome, Schnitzler syndrome, scleritis, scleroderma, Sjogren's syndrome, spondylarthropathy, Still's disease, stiff person syndrome, subacute bacterial endocarditis, Susac's syndrome, Sweet's syndrome, Sydenham chorea, sympathetic ophthalmia, Takayasu's arteritis, temporal arteritis, Tolosa-Hunt syndrome, transverse myelitis, ulcerative colitis, undifferentiated connective tissue disease, undifferentiated spondylarthropathy, vasculitis, vitiligo or Wegener's granulomatosis.

### 4. ABO Type and Rh Antigen Expression

Blood products can be classified into different groups according to the presence or absence of antigens on the surface of every red blood cell in a person's body (ABO Blood Type). The A, B, AB, and A1 antigens are determined by the sequence of oligosaccharides on the glycoproteins of erythrocytes. The genes in the blood group antigen group provide instructions for making antigen proteins. Blood group antigen proteins serve a variety of functions within the cell membrane of red blood cells. These protein functions include transporting other proteins and molecules into and out of the cell, maintaining cell structure, attaching to other cells and molecules, and participating in chemical reactions.

The Rhesus Factor (Rh) blood group is the second most important blood group system, after the ABO blood group system The Rh blood group system consists of 49 defined blood group antigens, among which five antigens, D, C, c. E, and e, are the most important. Rh(D) status of an individual is normally described with a positive or negative suffix after the ABO type. The terms “Rh factor,” “Rh positive,” and “Rh negative” refer to the Rh(D) antigen only. Antibodies to Rh antigens can be involved in hemolytic transfusion reactions and antibodies to the Rh(D) and Rh(c) antigens confer significant risk of hemolytic disease of the fetus and newborn. ABO antibodies develop in early life in every human. However, rhesus antibodies in Rh-humans typically develop only when the person is sensitized. This can occur, for example, by giving birth to a Rh+ baby or by receiving an Rh+ blood transfusion.

A, B, H, and Rh antigens are major determinants of histocompatibility benveen donor and recipient for blood, tissue and cellular transplantation. A glycosyltransferase activity encoded by the ABO gene is responsible for producing A, B, AB, O histo-blood group antigens, which are displayed on the surface of cells. Group A individuals encode an ABO gene product with specificity to produce a (1,3)N-acetylgalactosaminyltransferase activity and group B individuals with specificity to produce a(1, 3) galactosyltransferase activity. Type O individuals do not produce a functional galactosyltransferase at all and thus do not produce either modification. Type AB individuals harbor one copy of each and produce both types of modifications. The enzyme products of the ABO gene act on the H antigen as a substrate, and thus type O individuals whom lack ABO activity present an unmodified H antigen and are thus often referred to as type O(H).

The H antigen itself is the product of an a (1,2)fucosyltransferase enzyme, which is encoded by the FUT1 gene. In very rare individuals there exists a loss of the H antigen entirely as a result of a disruption of the FUT1 gene and no substrate will exist for ABO to produce A or B histo-blood types. These individuals are said to be of the Bombay histo-blood type. The Rh antigen is encoded by the RHD gene, and individuals who are Rh negative harbor a deletion or disruption of the RHD gene.

In some embodiments, the cells or population of cells provided herein are ABO type O Rh factor negative. In some embodiments, ABO type O Rh factor negative cells described herein are derived from an ABO type O Rh factor negative donor. In some embodiments. ABO type O Rh factor negative cells described herein are engineered to lack presentation of ABO type A, ABO type B, or Rh factor antigens. In some embodiments, ABO type O and/or Rh negative cells comprise partial or complete inactivation of an ABO gene (e.g., by deleterious variation of the ABO gene or by insertion of an exon 6 258delG variation of the ABO gene), and/or expression of an RHD gene is partially or fully inactivated by a deleterious variation of the RHD gene. In some embodiments, ABO type O Rh negative cells comprise partial or complete inactivation of a FUT1 gene and/or expression of an RHD gene is partially or fully inactivated by a deleterious variation of the RHD gene. In some embodiments, an engineered ABO type O and/or Rh factor negative cell is generated using gene editing to modify, for instance, a type A cell to a type O cell, a type B cell to a type O cell, a type AB cell to a type O cell, a type A+ cell to a type O− cell, a type A- cell to a type O− cell, a type AB+ cell to a type O− cell, a type AB− cell to a type O− cell, a type B+ cell to a type O− cell, and a type B− cell to a type O− cell. Exemplary engineered ABO type O Rh factor negative cells and methods of generating same are described in WO2021/146222, the content of which is herein incorporated by reference in its entirety.

In some embodiments, the cells or population of cells provided herein that comprise increased expression of CD46 and CD59 are ABO type A, ABO type B, or ABO type AB, and/or the cells or population of cells provided herein that comprise increased expression of CD46 and CD59 are Rh factor positive. In some embodiments, the cells that comprise increased expression of CD46 and CD59 can be administered to an ABO and/or Rh factor incompatible recipient patient without triggering a CDC reaction.

### 5. Sex Chromosomes

In certain aspects, cells having a sex chromosome may express certain antigens (e.g., Y antigens), and recipients may have a preexisting sensitivity to such antigens. For example, in some embodiments, a female who has been pregnant with a male fetus may reject cells from a male donor. Thus, in some embodiments, the donor is a male and the recipient is a male. In some embodiments, the donor is a female and the recipient is a female. In some embodiments, the engineered cell comprises a modification reducing expression of an antigen, such as Protocadherin Y and/or Neuroligin Y. In some embodiments, the gene encoding protocadheren Y (PCDH 11Y: Ensembl ID ENSG00000099715) is reduced or eliminated, e.g., knocked out, in the engineered cell. In some embodiments, the gene encoding Neuroligin Y (NLGN4Y; Ensembl ID ENSG00000165246) is reduced or eliminated, e.g., knocked out, in the engineered cell. Any method for reducing or eliminating expression of a gene can be used, such as any described herein. In some embodiments. PCDH11Y and/or NLGN4Y is reduced or eliminated in the engineered cell by nuclease-mediated gene editing methods such as using CRISPR/Cas systems.

### D. Gene Editing Systems for Insertion of Transgenes

In some aspects, the first transgene encoding a tolerogenic factor and/or the second transgene encoding a CAR can be integrated into the genome of a host cell (e.g., a T cell) using certain methods and compositions described herein.

### 1. Random Insertion

In some embodiments, the first transgene encoding a tolerogenic factor and/or the second transgene encoding a CAR can be inserted into a random genomic locus of a host cell. As known to a person skilled in the art, viral vectors, including, for example, retroviral vectors, lentiviral vectors, adenoviral vectors, and adeno-associated viral vectors, are commonly used to deliver genetic material into host cells and randomly insert the foreign or exogenous gene into the host cell genome to facilitate stable expression and replication of the gene.

### 2. Site-Directed Insertion (Knock-In)

In some embodiments, the first transgene encoding a tolerogenic factor and/or the second transgene encoding a CAR can be inserted into a specific genomic locus of the host cell. A number of gene editing methods can be used to insert a transgene into a specific genomic locus of choice. Gene editing is a type of genetic engineering in which a nucleotide sequence may be inserted, deleted, modified, or replaced in the genome of a living organism. In some embodiments, the gene editing technology can include systems involving nucleases, integrases, transposases, and/or recombinases. In some embodiments, the gene editing technology mediates single-strand breaks (SSB). In some embodiments, the gene editing technology mediates double-strand breaks (DSB), including in connection with non-homologous end-joining (NHEJ) or homology-directed repair (HDR). In some embodiments, the gene editing technology can include DNA-based editing or prime-editing. In some embodiments, the gene editing technology can include Programmable Addition via Site-specific Targeting Elements (PASTE). In some embodiments, the gene editing technology can include TnpB polypeptides. Many gene editing techniques generally utilize the innate mechanism for cells to repair double-strand breaks (DSBs) in DNA.

Eukaryotic cells repair DSBs by two primary repair pathways: non-homologous end-joining (NHEJ) and homology-directed repair (HDR). HDR typically occurs during late S phase or G2 phase, when a sister chromatid is available to serve as a repair template. NHEJ is more common and can occur during any phase of the cell cycle, but it is more error prone. In gene editing, NHEJ is generally used to produce insertion/deletion mutations (indels), which can produce targeted loss of function in a target gene by shifting the open reading frame (ORF) and producing alterations in the coding region or an associated regulatory region. HDR, on the other hand, is a preferred pathway for producing targeted knock-ins, knockouts, or insertions of specific mutations in the presence of a repair template with homologous sequences. Several methods are known to a skilled artisan to improve HDR efficiency, including, for example, chemical modulation (e.g., treating cells with inhibitors of key enzymes in the NHEJ pathway); timed delivery of the gene editing system at S and G2 phases of the cell cycle; cell cycle arrest at S and G2 phases; and introduction of repair templates with homology sequences. The methods provided herein may utilize HDR-mediated repair. NHEJ-mediated repair, or a combination thereof.

In some embodiments, the methods provided herein for HDR-mediated insertion utilize a site-directed nuclease, including, for example, zinc finger nucleases (ZFNs), transcription activator-like effector nucleases (TALENs), meganucleases, transposases, and clustered regularly interspaced short palindromic repeat (CRISPR)/Cas systems.

### A. ZFNS

ZFNs are fusion proteins comprising an array of site-specific DNA binding domains adapted from zinc finger-containing transcription factors attached to the endonuclease domain of the bacterial FokI restriction enzyme. A ZFN may have one or more (e.g., 1, 2, 3, 4, 5, 6, 7, 8, 9, 10, or more) of the DNA binding domains or zinc finger domains. See, e.g., Carroll et al., Genetics Society of America (2011) 188:773-782; Kim et al., Proc. Natl. Acad. Sci. USA (1996) 93:1156-1160. Each zinc finger domain is a small protein structural motif stabilized by one or more zinc ions and usually recognizes a 3- to 4-bp DNA sequence. Tandem domains can thus potentially bind to an extended nucleotide sequence that is unique within a cell's genome.

Various zinc fingers of known specificity can be combined to produce multi-finger polypeptides which recognize about 6, 9, 12, 15, or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Zinc fingers can be engineered to bind a predetermined nucleic acid sequence. Criteria to engineer a zinc finger to bind to a predetermined nucleic acid sequence are known in the art. See, e.g., Sera et al., Biochemistry (2002) 41:7074-7081; Liu et al., Bioinformatics (2008) 24:1850-1857.

ZFNs containing FokI nuclease domains or other dimeric nuclease domains function as a dimer. Thus, a pair of ZFNs are required to target non-palindromic DNA sites. The two individual ZFNs must bind opposite strands of the DNA with their nucleases properly spaced apart. See Bitinaite et al., Proc. Natl. Acad. Sci. USA (1998) 95:10570-10575. To cleave a specific site in the genome, a pair of ZFNs are designed to recognize two sequences flanking the site, one on the forward strand and the other on the reverse strand. Upon binding of the ZFNs on either side of the site, the nuclease domains dimerize and cleave the DNA at the site, generating a DSB with 5′ overhangs. HDR can then be utilized to introduce a specific mutation, with the help of a repair template containing the desired mutation flanked by homology arms. The repair template is usually an exogenous double-stranded DNA vector introduced to the cell. See Miller et al., Nat. Biotechnol. (2011) 29:143-148; Hockemeyer et al., Nat. Biotechnol. (2011) 29:731-734.

### B. TALENS

TALENs are another example of an artificial nuclease which can be used to edit a target gene. TALENs are derived from DNA binding domains termed TALE repeats, which usually comprise tandem arrays with 10 to 30 repeats that bind and recognize extended DNA sequences. Each repeat is 33 to 35 amino acids in length, with two adjacent amino acids (termed the repeat-variable di-residue, or RVD) conferring specificity for one of the four DNA base pairs. Thus, there is a one-to-one correspondence between the repeats and the base pairs in the target DNA sequences.

TALENs are produced artificially by fusing one or more TALE DNA binding domains (e.g., 1, 2, 3, 4, 5, 6, 7, 8, 9, 10 or more) to a nuclease domain, for example, a FokI endonuclease domain. See Zhang, Nature Biotech. (2011) 29:149-153. Several mutations to FokI have been made for its use in TALENs; these, for example, improve cleavage specificity or activity. See Cermak et al., Nucl. Acids Res. (2011) 39:e82; Miller et al., Nature Biotech. (2011) 29:143-148; Hockemeyer et al., Nature Biotech. (2011) 29:731-734; Wood et al., Science (2011) 333:307; Doyon et al., Nature Methods (2010) 8:74-79; Szczepek et al., Nature Biotech (2007)25:786-793; Guo et al., J. Mol. Biol. (2010) 200:96. The FokI domain functions as a dimer, requiring two constructs with unique DNA binding domains for sites in the target genome with proper orientation and spacing. Both the number of amino acid residues between the TALE DNA binding domain and the FokI nuclease domain and the number of bases between the two individual TALEN binding sites appear to be important parameters for achieving high levels of activity. Miller et al., Nature Biotech. (2011) 29:143-148.

By combining engineered TALE repeats with a nuclease domain, a site-specific nuclease can be produced specific to any desired DNA sequence. Similar to ZFNs, TALENs can be introduced into a cell to generate DSBs at a desired target site in the genome, and so can be used to knock out genes or knock in mutations in similar, HDR-mediated pathways. See Boch, Nature Biotech. (2011) 29:135-136; Boch et al., Science (2009)326:1509-1512; Moscou et al., Science (2009) 326:3501.

### C. Meganucleases

Meganucleases are enzymes in the endonuclease family which are characterized by their capacity to recognize and cut large DNA sequences (from 14 to 40 base pairs). Meganucleases are grouped into families based on their structural motifs which affect nuclease activity and/or DNA recognition. The most widespread and best known meganucleases are the proteins in the LAGLIDADG family, which owe their name to a conserved amino acid sequence. See Chevalier et al., Nucleic Acids Res. (2001) 29(18): 3757-3774. On the other hand, the GIY-YIG family members have a GIY-YIG module, which is 70-100 residues long and includes four or five conserved sequence motifs with four invariant residues, two of which are required for activity. See Van Roey et al., Nature Struct. Biol. (2002) 9:806-811. The His-Cys family meganucleases are characterized by a highly conserved series of histidines and cysteines over a region encompassing several hundred amino acid residues. See Chevalier et al., Nucleic Acids Res. (2001) 29(18):3757-3774. Members of the NHN family are defined by motifs containing two pairs of conserved histidines surrounded by asparagine residues. See Chevalier et al., Nucleic Acids Res. (2001) 29(18):3757-3774.

Because the chance of identifying a natural meganuclease for a particular target DNA sequence is low due to the high specificity requirement, various methods including mutagenesis and high throughput screening methods have been used to create meganuclease variants that recognize unique sequences. Strategies for engineering a meganuclease with altered DNA-binding specificity, e.g., to bind to a predetermined nucleic acid sequence are known in the art. See, e.g., Chevalier et al., Mol. Cell. (2002) 10:895-905; Epinat et al., Nucleic Acids Res (2003) 31:2952-2962; Silva et al., J Mol. Biol. (2006) 361:744-754; Seligman et al., Nucleic Acids Res (2002) 30:3870-3879; Sussman et al., J Mol Biol (2004) 342:31-41: Doyon et al., J Am Chem Soc (2006) 128:2477-2484; Chen et al., Protein Eng Des Sel (2009) 22:249-256; Arnould et al., J Mol Biol. (2006) 355:443-458; Smith et al., Nucleic Acids Res. (2006) 363(2):283-294.

Like ZFNs and TALENs. Meganucleases can create DSBs in the genomic DNA, which can create a frame-shift mutation if improperly repaired, e.g., via NHEJ, leading to a decrease in the expression of a target gene in a cell. Alternatively, foreign DNA can be introduced into the cell along with the meganuclease. Depending on the sequences of the foreign DNA and chromosomal sequence, this process can be used to modify the target gene. See Silva et al., Current Gene Therapy (2011) 11:11-27.

### D. Transposases

Transposases are enzymes that bind to the end of a transposon and catalyze its movement to another part of the genome by a cut and paste mechanism or a replicative transposition mechanism. By linking transposases to other systems such as the CRISPR/Cas system, new gene editing tools can be developed to enable site specific insertions or manipulations of the genomic DNA. There are two known DNA integration methods using transposons which use a catalytically inactive Cas effector protein and Tn7-like transposons. The transposase-dependent DNA integration does not provoke DSBs in the genome, which may guarantee safer and more specific DNA integration.

### E. CRISPR/CAS

The CRISPR system was originally discovered in prokaryotic organisms (e.g., bacteria and archaea) as a system involved in defense against invading phages and plasmids that provides a form of acquired immunity. Now it has been adapted and used as a popular gene editing tool in research and clinical applications.

CRISPR/Cas systems generally comprise at least two components: one or more guide RNAs (gRNAs) and a Cas protein. The Cas protein is a nuclease that introduces a DSB into the target site. CRISPR-Cas systems fall into two major classes: class 1 systems use a complex of multiple Cas proteins to degrade nucleic acids; class 2 systems use a single large Cas protein for the same purpose. Class 1 is divided into types I, III, and IV; class 2 is divided into types II, V, and VI. Different Cas proteins adapted for gene editing applications include, but are not limited to, Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. See, e.g., Jinek et al., Science (2012) 337 (6096):816-821; Dang et al., Genome Biology (2015) 16:280; Ran et al., Nature (2015) 520:186-191; Zetsche et al., Cell (2015) 163:759-771; Strecker et al., Nature Comm. (2019) 10:212; Yan et al., Science (2019) 363:88-91. The most widely used Cas9 is a type II Cas protein and is described herein as illustrative. These Cas proteins may be originated from different source species. For example, Cas9 can be derived from S. pyogenes or S. aureus.

In the original microbial genome, the type II CRISPR system incorporates sequences from invading DNA between CRISPR repeat sequences encoded as arrays within the host genome. Transcripts from the CRISPR repeat arrays are processed into CRISPR RNAs (crRNAs) each harboring a variable sequence transcribed from the invading DNA, known as the “protospacer” sequence, as well as part of the CRISPR repeat. Each crRNA hybridizes with a second transactivating CRISPR RNA (tracrRNA), and these two RNAs form a complex with the Cas9 nuclease. The protospacer-encoded portion of the crRNA directs the Cas9 complex to cleave complementary target DNA sequences, provided that they are adjacent to short sequences known as “protospacer adjacent motifs” (PAMs).

While the foregoing description has focused on Cas9 nuclease, it should be appreciated that other RNA-guided nucleases exist which utilize gRNAs that differ in some ways from those described to this point. For instance, Cpf1 (CRISPR from Prevotella and Franciscella 1; also known as Cas12a) is an RNA-guided nuclease that only requires a crRNA and does not need a tracrRNA to function.

Since its discovery, the CRISPR system has been adapted for inducing sequence specific DSBs and targeted genome editing in a wide range of cells and organisms spanning from bacteria to eukaryotic cells including human cells. In its use in gene editing applications, artificially designed, synthetic gRNAs have replaced the original crRNA:tracrRNA complexes, including in certain embodiments via a single gRNA. For example, the gRNAs can be single guide RNAs (sgRNAs) composed of a crRNA, a tetraloop, and a tracrRNA. The crRNA usually comprises a complementary region (also called a spacer, usually about 20 nucleotides in length) that is user-designed to recognize a target DNA of interest. The tracrRNA sequence comprises a scaffold region for Cas nuclease binding. The crRNA sequence and the tracrRNA sequence are linked by the tetraloop and each have a short repeat sequence for hybridization with each other, thus generating a chimeric sgRNA. One can change the genomic target of the Cas nuclease by simply changing the spacer or complementary region sequence present in the gRNA. The complementary region will direct the Cas nuclease to the target DNA site through standard RNA-DNA complementary base pairing rules.

In order for the Cas nuclease to function, there must be a PAM immediately downstream of the target sequence in the genomic DNA. Recognition of the PAM by the Cas protein is thought to destabilize the adjacent genomic sequence, allowing interrogation of the sequence by the gRNA and resulting in gRNA-DNA pairing when a matching sequence is present. The specific sequence of PAM varies depending on the species of the Cas gene. For example, the most commonly used Cas9 nuclease derived from S. pyogenes recognizes a PAM sequence of 5′-NGG-3′ or, at less efficient rates, 5′-NAG-3′, where “N” can be any nucleotide. Other Cas nuclease variants with alternative PAMs have also been characterized and successfully used for genome editing, which are summarized in Table 1a.

In some embodiments, Cas nucleases may comprise one or more mutations to alter their activity, specificity, recognition, and/or other characteristics. For example, the Cas nuclease may have one or more mutations that alter its fidelity to mitigate off-target effects (e.g., eSpCas9, SpCas9-HF1, HvpaSpCas9, HeFSpCas9, and evoSpCas9 high-fidelity variants of SpCas9). For another example, the Cas nuclease may have one or more mutations that alter its PAM specificity.

In some embodiments. CRISPR systems of the present disclosure comprise TnpB polypeptides. In some embodiments, TnpB polypeptides may comprise a Ruv-C-like domain. The RuvC domain may be a split RuvC domain comprising RuvC-I, RuvC-II, and RuvC-III subdomains. In some embodiments, a TnpB may further comprise one or more of a HTH domain, a bridge helix domain and a zinc finger domain. TnpB polypeptides do not comprise an HNH domain. In some embodiments, a TnpB protein comprises, starting at the N-terminus: a HTH domain, a RuvC-I subdomain, a bridge helix domain, a RuvC-II sub-domain, a zinger finger domain, and a RuvC-III sub-domain. In some embodiments, a RuvC-III sub-domain forms the C-terminus of a TnpB polypeptide. In some embodiments, a TnpB polypeptide is from Epsilonproteobacteria bacterium, Actinoplanes lobatus strain DSM 43150, Actinomadura celluolosilytica strain DSM 45823. Actinomadura namibiensis strain DSM 44197, Alicyclobacillus macrosprangiidus strain DSM 17980, Lipingzhangella halophila strain DSM 102030, or Ktedonobacter racemifer. In some embodiments, a TnpB polypeptide is from Ktedonobacter racemifer, or comprises a conserved RNA region with similarity to the 5′ ITR of K. racemifer TnpB loci. In some embodiments, a TnpB may comprise a Fanzor protein, a TnpB homolog found in eukaryotic genomes. In some embodiments, a CRISPR system comprising a TnpB polypeptide binds a target adjacent motif (TAM) sequence 5′ of a target polynucleotide. In some embodiments, a TAM is a transposon-associated motif. In some embodiments, a TAM sequence comprises TCA. In some embodiments, a TAM sequence comprises TTCAN. In some embodiments, a TAM sequence comprises TTGAT. In some embodiments, a TAM sequence comprises ATAAA.

In certain embodiments, the first and/or the second transgene may function as a DNA repair template to be integrated into the target site through HDR in associated with a gene editing system (e.g., the CRISPR/Cas system) as described. Generally, the transgene to be inserted would comprise at least the expression cassette encoding the protein of interest (e.g., the tolerogenic factor or CAR) and would optionally also include one or more regulatory elements (e.g., promoters, insulators, enhancers). In certain of these embodiments, the transgene to be inserted would be flanked by homologous sequence immediately upstream and downstream of the target, i.e., left homology arm (LHA) and right homology arm (RHA), specifically designed for the target genomic locus to serve as template for HDR. The length of each homology arm is generally dependent on the size of the insert being introduced, with larger insertions requiring longer homology arms.

In some embodiments, target-primed reverse transcription (TPRT) or prime editing may be used to engineer exogenous genes, such as exogenous transgenes encoding a tolerogenic factor (e.g., CD47) into specific loci. In some embodiments, prime editing mediates targeted insertions, deletions, all 12 possible base-to-base conversions, and combinations thereof in human cells without requiring DSBs or donor DNA templates.

Prime editing is a genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand of the target site to be edited (with the exception that it includes the desired edit). Through DNA repair and/or replication machinery, the endogenous strand of the target site is replaced by the newly synthesized replacement strand containing the desired edit. In some cases, prime editing may be thought of as a “search-and- replace” genome editing technology since the prime editors search and locate the desired target site to be edited, and encode a replacement strand containing a desired edit which is installed in place of the corresponding target site endogenous DNA strand at the same time. For example, prime editing can be adapted for conducting precision CRISPR/Cas-based genome editing in order to bypass double stranded breaks. In some embodiments, a homologous protein is or encodes for a Cas protein-reverse transcriptase fusions or related systems to target a specific DNA sequence with a guide RNA, generate a single strand nick at the target site, and use the nicked DNA as a primer for reverse transcription of an engineered reverse transcriptase template that is integrated with the guide RNA. In some embodiments, a prime editor protein is paired with two prime editing guide RNAs (pegRNAs) that template the synthesis of complementary DNA flaps on opposing strands of genomic DNA, resulting in the replacement of endogenous DNA sequence between the PE-induced nick sites with pegRNA-encoded sequences.

In some embodiments, a gene editing technology is associated with a prime editor that is a reverse transcriptase, or any DNA polymerase known in the art. Thus, in one aspect, a prime editor may comprise Cas9 (or an equivalent napDNAbp) which is programmed to target a DNA sequence by associating it with a specialized guide RNA (i.e., PEgRNA) containing a spacer sequence that anneals to a complementary protospacer in the target DNA. Such methods include any disclosed in Anzalone et al., (doi.org/l0.1038/s41586-019-1711-4), or in PCT publication Nos. WO2020191248, WO2021226558, or WO2022067130, which are hereby incorporated in their entirety.

In some embodiments, the base editing technology may be used to introduce single-nucleotide variants (SNVs) into DNA or RNA in living cells. Base editing is a CRISPR-Cas9-based genome editing technology that allows the introduction of point mutations in RNAs or DNAs without generating DSBs. Base editors (BEs) are typically fusions of a Cas (“CRISPR-associated”) domain and a nucleobase modification domain (e.g., a natural or evolved deaminase, such as a cytidine deaminase that include APOBEC1 (“apolipoprotein B mRNA editing enzyme, catalytic polypeptide 1”), CDA (“cytidine deaminase”), and AID (“activation-induced cytidine deaminase”)) domains. In some embodiments, base editors may also include proteins or domains that alter cellular DNA repair processes to increase the efficiency and/or stability of the resulting single-nucleotide change. Two major classes of base editors have been developed: cytidine base editors (CBEs) (e.g., BE4) that allow C:G to T:A conversions and adenine base editors (ABEs) (e.g., ABE7.10) that allow A:T to G:C conversions. Base editors are composed by a catalytically dead Cas9 (dCas9) or a nickase Cas9 (nCas9) fused to a deaminase and guided by a sgRNA to the locus of interest. The d/nCas9 recognizes a specific PAM sequence and the DNA unwinds thanks to the complementarity between the sgRNA and the DNA sequence usually located upstream of the PAM (also called protospacer). Then, the opposite DNA strand is accessible to the deaminase that converts the bases located in a specific DNA stretch of the protospacer. Compared to HDR-based strategies, base editing is a promising tool to precisely correct genetic mutations as it avoids gene disruption by NHEJ associated with failed HDR-mediated gene correction. Rat deaminase APOBEC1 (rAPOBEC1) fused to deactivated Cas9 (dCas9) has been used to successfully convert cytidines to thymidines upstream of the PAM of the sgRNA. In some embodiments, this first BE system was optimized by changing the dCas9 to a “nickase” Cas9 D10A, which nicks the strand opposite the deaminated cytidine. Without being bound by theory, this is expected to initiate long-patch base excision repair (BER), where the deaminated strand is preferentially used to template the repair to produce a U:A base pair, which is then converted to T:A during DNA replication.

In some embodiments, a base editor is a nucleobase editor containing a first DNA binding protein domain that is catalytically inactive, a domain having base editing activity, and a second DNA binding protein domain having nickase activity, where the DNA binding protein domains are expressed on a single fusion protein or are expressed separately (e.g., on separate expression vectors). In some embodiments, a base editor is a fusion protein comprising a domain having base editing activity (e.g., cytidine deaminase or adenosine deaminase), and two nucleic acid programmable DNA binding protein domains (napDNAbp), a first comprising nickase activity and a second napDNAbp that is catalytically inactive, wherein at least the two napDNAbp are joined by a linker. In some embodiments, a base editor is a fusion protein that comprises a DNA domain of a CRISPR-Cas (e.g., Cas9)having nickase activity (nCas; nCas9), a catalytically inactive domain of a CRISPR-Cas protein (e.g., Cas9) having nucleic acid programmable DNA binding activity (dCas; e.g., dCas9), and a deaminase domain, wherein the dCas is joined to the nCas by a linker, and the dCas is immediately adjacent to the deaminase domain. In some embodiments, a base editor is an adenine-to-thymine or “ATBE” (or thymine-to-adenine or “TABE”) transversion base editor. Exemplary base editor and base editor systems include any as described in patent publication Nos. US20220127622, US20210079366, US20200248169, US20210093667, US20210071163, WO2020181202, WO2021158921, WO2019126709, WO2020181178, WO2020181195, WO2020214842, WO2020181193, which are hereby incorporated in their entirety.

In some embodiments, a gene editing technology is Programmable Addition via Site-specific Targeting Elements (PASTE). In some aspects, PASTE is platform in which genomic insertion is directed via a CRISPR-Cas9 nickase fused to both a reverse transcriptase and serine integrase. As described in Ioannidi et al. (doi.org/10.1101/2021.11.01.466786), PASTE does not generate double stranded breaks, but allows for integration of sequences as large as ˜36 kb. In some embodiments, a serine integrase can be any known in the art. In some embodiments, a serine integrase has sufficient orthogonality such that PASTE can be used for multiplexed gene integration, simultaneously integrating at least two different genes at at least two genomic loci. In some embodiments, PASTE has editing efficiencies comparable to or better than those of homology directed repair or non-homologous end joining based integration, with activity in non-dividing cells and fewer detectable off-target events.

### 3. Genomic Lod for Insertion of the First Transgene

In some embodiments, the genomic locus for site-directed insertion of the first transgene encoding a tolerogenic factor is an endogenous TCR gene locus. In some embodiments, the endogenous TCR gene locus is selected from the group consisting of a TRAC locus, a TRBC1 locus, and a TRBC2 locus. The specific site for insertion within a gene locus may be located within any suitable region of the gene, including but not limited to a gene coding region (also known as a coding sequence or “CDS”), an exon, an intron, a sequence spanning a portion of an exon and a portion of an adjacent intron, or a regulatory region (e.g., promoter, enhancer). In some embodiments, the insertion occurs in one allele of the specific genomic locus. In some embodiments, the insertion occurs in both alleles of the specific genomic locus. In either of these embodiments, the orientation of the transgene inserted into the target genomic locus can be either the same or the reverse of the direction of the endogenous gene in that locus.

### A. TRAC

TCRs recognize foreign antigens which have been processed as small peptides and bound to MHC molecules at the surface of antigen presenting cells (APC). Each TCR is a dimer consisting of one alpha and one beta chain (most common) or one delta and one gamma chain. The genes encoding the TCR alpha chain are clustered on chromosome 14. The TCR alpha chain is formed when one of at least 70 variable (V) genes, which encode the N-terminal antigen recognition domain, rearranges to 1 of 61 joining (J) gene segments to create a functional variable region that is transcribed and spliced to a constant region gene segment encoding the C-terminal portion of the molecule. The beta chain, on the other hand, is generated by recombination of the V, D (diversity), and J segment genes.

The TRAC gene encodes the TCR alpha chain constant region. The human TRAC gene resides on chromosome 14 at 22,547,506-22,552,156, forward strand. The TRAC genomic sequence is set forth in Ensembl ID ENSG00000277734.

### B. TRBC1 and TRBC2

The TRBC gene encodes the TCR beta chain constant region. TRBC1 and TRBC2 are analogs of the same gene, and T cells mutually exclusively express either TRBC1 and TRBC2. The human TRBC1 gene resides on chromosome 7 at 142,791,694-142,793,368, forward strand, and its genomic sequence is set forth in Ensembl ID ENSG00000211751. The human TRBC2 gene resides on chromosome 7 at 142,801,041-142,802.748, forward strand, and its genomic sequence is set forth in Ensembl ID ENSG00000211772.

### 4. Genomic Lod for Insertion of the Second Transgene

In some embodiments, the genomic locus for insertion of the second transgene encoding a CAR can be a random locus (by random insertion) or a specific locus (by site-directed insertion). If a specific locus is desired, it can be the same as or a different locus from that of the first transgene. In some embodiments, the genomic locus for insertion of the second transgene encoding a CAR is a specific locus selected from the group consisting of a TRAC locus, a TRBC1 locus, a TRBC2 locus, a B2M locus, a CIITA locus, and a safe harbor locus. Non-limiting examples of safe harbor loci include, but are not limited to, an AAVS1 (also known as PPP1R12C), ABO, CCR5, CLYBL, CXCR4, F3 (also known as CD142), FUT1, HMGB1. KDM5D, LRP1 (also known as CD91), MICA, MICB, RHD, ROSA26, and SHS231 gene locus. In some embodiments, the genomic locus for insertion of the second transgene encoding a CAR is a specific locus comprising a TRAC locus, a TRBC1 locus, a TRBC2 locus, a B2M locus, a CIITA locus, an AAVS1 (also known as PPPIR12C) locus, an ABO locus, a CCR5 locus, a CLYBL locus, aCXCR4 locus, an F3 (also known as CD142) locus, a FUT1 locus, an HMGB1 locus, a KDM5D locus, an LRP1 (also known as CD91) locus, a MICA locus, an MICB locus, an RHD locus, a ROSA26 locus, or an SHS231 locus. The second transgene can be inserted within any suitable region of any of the described locus, including but not limited to a gene coding region (also known as a coding sequence or “CDS”), an exon, an intron, a sequence spanning a portion of an exon and a portion of an adjacent intron, or a regulatory region (e.g., promoter, enhancer). In some embodiments, the insertion occurs in one allele of the genomic locus. In some embodiments, the insertion occurs in both alleles of the genomic locus. In either of these embodiments, the orientation of the transgene inserted into the genomic locus can be either the same or the reverse of the direction of the original gene in that locus. In some embodiments, the second transgene is inserted with the first transgene such as the first transgene and the second transgene are carried by a polycistronic vector.

5. Guide RNAs (gRNAs) for Site-Directed Insertion

In some embodiments, provided are gRNAs for use in site-directed insertion of a transgene in according to various embodiments provided herein, especially in association with the CRISPR/Cas system. The gRNAs comprise a crRNA sequence, which in turn comprises a complementary region (also called a spacer) that recognizes and binds a complementary target DNA of interest. The length of the spacer or complementary region is generally between 15 and 30 nucleotides, usually about 20 nucleotides in length, although will vary based on the requirements of the specific CRISPR/Cas system. In certain embodiments, the spacer or complementary region is fully complementary to the target DNA sequence. In other embodiments, the spacer is partially complementary to the target DNA sequence, for example at least 80%, 85%, 90%, 95%, 98%, or 99% complementary.

In certain embodiments, the gRNAs provided herein further comprise a tracrRNA sequence, which comprises a scaffold region for binding to a nuclease. The length and/or sequence of the tracrRNA may vary depending on the specific nuclease being used for editing. In certain embodiments, nuclease binding by the gRNA does not require a tracrRNA sequence. In those embodiments where the gRNA comprises a tracrRNA, the crRNA sequence may further comprise a repeat region for hybridization with complementary sequences of the tracrRNA.

In some embodiments, the gRNAs provided herein comprise two or more gRNA molecules, for example, a crRNA and a tracrRNA, as two separate molecules. In other embodiments, the gRNAs are single guide RNAs (sgRNAs), including sgRNAs comprising a crRNA and a tracrRNA on a single RNA molecule. In certain of these embodiments, the crRNA and tracrRNA are linked by an intervening tetraloop.

In some embodiments, one gRNA can be used in association with a site-directed nuclease for targeted editing of a gene locus of interest. In other embodiments, two or more gRNAs targeting the same gene locus of interest can be used in association with a site-directed nuclease.

In some embodiments, exemplary gRNAs (e.g., sgRNAs) for use with various common Cas nucleases that require both a crRNA and tracrRNA, including Cas9 and Cas12b (C2c1), are provided in Table 18. See, e.g., Jinek et al., Science (2012) 337 (6096):816-821; Dang et al., Genome Biology (2015) 16:280; Ran et al., Nature (2015) 520:186-191; Strecker et al., Nature Comm. (2019) 10:212. For each exemplary gRNA, sequences for different portions of the gRNA, including the complementary region or spacer, crRNA repeat region, tetraloop, and tracrRNA, are shown. In some embodiments, the gRNA comprises all or a portion of the nucleotide sequences set forth in SEQ ID NOs: 108A, 109A, 110A, 111A. In some embodiments, the gRNA comprises all or a portion of the nucleotide sequences set forth in SEQ ID NOs: 112A, 113A, 114A, 115A. In some embodiments, the gRNA comprises all or a portion of the nucleotide sequences set forth in SEQ ID NOs: 116A, 117A, I18A, 119A. In some embodiments, the gRNA comprises all or a portion of the nucleotide sequences set forth in SEQ ID NOs: 120A, 121A, 122A, 123A.

In some embodiments, the gRNA comprises a crRNA repeat region comprising, consisting of, or consisting essentially of the nucleotide sequence set forth in SEQ ID NO:109A, SEQ ID NO:113A, SEQ ID NO:117A, or SEQ ID NO:122A. In some embodiments, the gRNA comprises a tetraloop comprising, consisting of, or consisting essentially of the nucleotide sequence set forth in SEQ ID NO:110A or SEQ ID NO:121A. In some embodiments, the gRNA comprises a tracrRNA comprising, consisting of, or consisting essentially of the nucleotide sequence set forth in SEQ ID NO:111A, SEQ ID NO: 115A, SEQ ID NO:119A, or SEQ ID NO:120A.

TABLE 18
Exemplary gRNA structure and sequence for CRISPR/Cas

| SEQ ID NO: | Sequence (5′→3′) | Description |
|---|---|---|
| 108A | nnnnnnnnnnnnnnnnnnnn | Exemplary spCas9 1 |
| | | Complementary region |
| | | (spacer) |
| 109A | guuuuagagcua | Exemplary spCas9 1 |
| | | crRNA repeat region |
| 110A | gaaa | Exemplary spCas9 1 |
| | | tetraloop |
| 111A | uagcaaguuaaaauaaggcuaguccguuaucaacuugaaaaa | Exemplary spCas9 1 |
| | guggcaccgagucggugcuuuuuu | tracrRNA |
| 112A | nnnnnnnnnnnnnnnnnnnn | Exemplary spCas9 2 |
| | | Complementary region |
| | | (spacer) |
| 113A | guuusagagcuaugcug | Exemplary spCas9 2 |
| | | crRNA repeat region |
| 114A | gaaa | Exemplary spCas9 2 |
| | | tetraloop |
| 115A | cagcauagcaaguusaaauaaggcuaguccguuaucaacuug | Exemplary spCas9 2 |
| | aaaaaguggcaccgagucggugcuuuuuu | tracrRNA |
| 116A | nnnnnnnnnnnnnnnnnnnn | Exemplary saCas9 |
| | | Complementary region |
| | | (spacer) |
| 117A | guuuuaguacucug | Exemplary saCas9 crRNA |
| | | repeat region |
| 118A | gaaa | Exemplary saCas9 |
| | | tetraloop |
| 119A | cagaaucuacuaaaacaaggcaaaaugccguguuuaucucgu | Exemplary |
| | caacuuguuggcgagauuuuuu | tracrRNA |
| 120A | gucgucuauaggacggcgaggacaacgggaagugccaaugug | Exemplary AkCas12b |
| | cucuuuccaagagcaaacaccccguuggcuucaagaugaccgc | tracrRNA |
| | ucg | |
| 121A | aaaa | Exemplary AkCas12b |
| | | tetraloop |
| 122A | cgagcggucugagaaguggcacu | Exemplary AkCas12b |
| | | crRNA repeat region |
| 123A | nnnnnnnnnnnnnnnnnnnn | Exemplary AkCas12b |
| | | Complementary region |
| | | (spacer) |

s = c or g; n = any base

In some embodiments, the gRNA comprises a complementary region specific to a target gene locus of interest, for example, the TRAC locus, the TRBC1 locus, the TRBC2 locus, B2M locus, the CIITA locus, or a safe harbor locus selected from the group consisting of an AAVS1, ABO, CCR5, CLYBL, CXCR4, F3, FUT1, HMGB1, KDM5D, LRP1, MICA, MICB, RHD, ROSA26, and SHS231 gene locus. The complementary region may bind a sequence in any region of the target gene locus, including for example, a CDS, an exon, an intron, a sequence spanning a portion of an exon and a portion of an adjacent intron, or a regulatory region (e.g., promoter, enhancer). Where the target sequence is a CDS, exon, intron, or sequence spanning portions of an exon and intron, the CDS, exon, intron, or exon/intron boundary may be defined according to any splice variant of the target gene. In some embodiments, the genomic locus targeted by the gRNA is located within 4000 bp, within 3500 bp, within 3000 bp, within 2500 bp, within 2000 bp, within 1500 bp, within 1000 bp, or within 500 bp of any of the loci or regions thereof as described. Further provided herein are compositions comprising one or more gRNAs provided herein and a Cas protein or a nucleotide sequence encoding a Cas protein. In certain of these embodiments, the one or more gRNAs and a nucleotide sequence encoding a Cas protein are comprised within a vector, for example, a viral vector.

In some embodiments, provided are methods of identifying new loci and/or gRNA sequences for use in the site-directed genomic insertion approaches as described. For example, for CRISPR/Cas systems, when an existing gRNA for a particular locus (e.g., within an endogenous TCR gene locus) is known, an “inch worming” approach can be used to identify additional loci for targeted insertion of transgenes by scanning the flanking regions on either side of the locus for PAM sequences, which usually occurs about every 100 base pairs (bp) across the genome. The PAM sequence will depend on the particular Cas nuclease used because different nucleases usually have different corresponding PAM sequences. The flanking regions on either side of the locus can be between about 500 to 4000 bp long, for example, about 500 bp, about 1000 bp, about 1500 bp, about 2000 bp, about 2500 bp, about 3000 bp, about 3500 bp, or about 4000 bp long. When a PAM sequence is identified within the search range, a new guide can be designed according to the sequence of that locus for use in site-directed insertion of transgenes. Although the CRISPR/Cas system is described as illustrative, any gene editing approaches as described can be used in this method of identifying new loci, including those using ZFNs, TALENs, meganucleases, and transposases.

In some embodiments, the activity, stability, and/or other characteristics of gRNAs can be altered through the incorporation of chemical and/or sequential modifications. As one example, transiently expressed or delivered nucleic acids can be prone to degradation by, e.g., cellular nucleases. Accordingly, the gRNAs described herein can contain one or more modified nucleosides or nucleotides which introduce stability toward nucleases. While not being bound by a particular theory, it is believed that certain modified gRNAs described herein can exhibit a reduced innate immune response when introduced into a population of cells, particularly the cells of the present technology. As used herein, the term “innate immune response” includes a cellular response to exogenous nucleic acids, including single stranded nucleic acids, generally of viral or bacterial origin, which involves the induction of cytokine expression and release, e.g., the interferons, and cell death. Other common chemical modifications of gRNAs to improve stabilities, increase nuclease resistance, and/or reduce immune response include 2′-O-methyl modification, 2′-fluoro modification, 2′-O-methyl phosphorothioate linkage modification, and 2′-O-methyl 3′ thioPACE modification.

One common 3′ end modification is the addition of a poly(A) tract comprising one or more (and typically 5-200) adenine (A) residues. The poly(A) tract can be contained in the nucleic acid sequence encoding the gRNA or can be added to the gRNA during chemical synthesis or following in vitro transcription using a polyadenosine polymerase (e.g., E. coli poly(A) polymerase). In vivo, poly(A) tracts can be added to sequences transcribed from DNA vectors through the use of polyadenylation signals. Examples of such signals are provided in Maeder. Other suitable gRNA modifications include, without limitations, those described in U.S. Patent Application No. US 2017/0073674 A1 and International Publication No. WO 2017/165862 A1, the entire contents of each of which are incorporated by reference herein.

In some embodiments, a tool for designing a gRNA as disclosed herein comprises: Benchling, Broad Institute GPP, CasOFFinder, CHOPCHOP, CRISPick, CRISPOR, Deskgen, E-CRISP, Geneious, Guides, Horizon Discovery, IDT, Off-Spotter, Synthego, or TrueDesign (ThermoFisher). One of ordinary skill in the art would understand that a tool that predicts both activity and specificity (e.g., to limit off-target modification) would be useful for designing a gRNA in certain instances as disclosed herein.

F. Delivery of Gene Editing Systems into a Host Cell

In some embodiments, provided are compositions comprising one or more components of a gene editing system described herein, including one or more gRNAs, a site-directed nuclease (e.g., a Cas nuclease) or a nucleotide sequence encoding a site-directed nuclease protein, and a transgene for targeted insertion. In some embodiments, the compositions are formulated for delivery into a cell.

In some embodiments, components of a gene editing system provided herein, including one or more gRNAs, a site-directed nuclease (e.g., a Cas nuclease) or a nucleotide sequence encoding a site-directed nuclease protein, and a transgene (e.g., the first transgene encoding a tolerogenic factor and/or the second transgene encoding a CAR) for targeted insertion, may be delivered into a cell in the form of a delivery vector. The delivery vector can be any type of vector suitable for introduction of nucleotide sequences into a cell, including, for example, plasmids, adenoviral vectors, adeno-associated viral (AAV) vectors, retroviral vectors, lentiviral vectors, phages, and HDR-based donor vectors. The different components may be introduced into a cell together or separately, and may be delivered in a single vector or multiple vectors.

In some embodiments, the delivery vector may be introduced into a cell by any known method in the field, including, for example, viral transformation, calcium phosphate transfection, lipid-mediated transfection, DEAE-dextran, electroporation, microinjection, nucleoporation, liposomes, nanoparticles, or other methods.

In some embodiments, the present technology provides compositions comprising a delivery vector according to various embodiments disclosed herein. In some embodiments, the compositions may further comprise one or more pharmaceutically acceptable carriers, excipients, preservatives, or a combination thereof. A “pharmaceutically acceptable carrier or excipient” refers to a pharmaceutically acceptable material, composition, or vehicle that is involved in carrying or transporting a compound of interest from one tissue, organ, or portion of the body to another tissue, organ, or portion of the body. For example, the carrier or excipient may be a liquid or solid filler, diluent, excipient, solvent, or encapsulating material, or some combination thereof. Each component of the carrier or excipient must be “pharmaceutically acceptable,” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits. Suitable excipients include water, saline, dextrose, glycerol, or the like and combinations thereof. In some embodiments, compositions comprising cells as disclosed herein further comprise a suitable infusion media.

In some embodiments, provided are cells or compositions thereof comprising one or more components of a gene editing system described herein, including one or more gRNAs, a site-directed nuclease (e.g., a Cas nuclease) or a nucleotide sequence encoding a site-directed nuclease protein, and a transgene for targeted insertion.

### E. Exemplary Embodiments of Engineered Cells

As set out above, methods for profiling a population of cells for donor capability disclosed herein may advantageously form a part of an overall method for manufacturing a cell therapy product. It will be understood that, in the process of manufacturing a cell therapy, certain modifications may be introduced to the cell that are considered desirable for the cell therapy product e.g. to assist the cell to evade immune recognition. In some embodiments, the engineered cells and populations thereof exhibit reduced expression of one or more molecules of the MHC class I and/or MHC class II molecules. In some embodiments, the engineered cells and populations thereof exhibit increased expression of at least one tolerogenic factor. In some embodiments, the engineered cells and populations thereof exhibit reduced expression of one or more molecules of the MHC class I and/or MHC class II molecules and increased expression of at least one tolerogenic factor.

In some embodiments, the cells (e.g., engineered beta islet cells, hepatocytes, or other cell types that come into contact with the blood during transplantation) and populations thereof exhibit increased expression of CD47, have increased expression of at least one tolerogenic factor, and reduced expression of one or more molecules of the MHC class I complex. In some embodiments, the cells and populations thereof exhibit increased expression of CD47, increased expression of at least one tolerogenic factor, and reduced expression of one or more molecules of the MHC class I and/or MHC class II complexes. In some embodiments, the engineered cells express one or more exogenous complement inhibitor polypeptides selected from CD46. CD59, CD55, and any combinations thereof. In some embodiments, at least one tolerogenic factor is at least one of the tolerogenic factors described herein.

### F. Therapeutic Cells Differentiated from Hypoimmunogenic Pluripotent Stem Cells

Provided herein are hypoimmunogenic cells including, cells derived from pluripotent stem cells, that evade immune recognition. In some embodiments, the cells do not activate an innate and/or an adaptive immune response in the patient or subject (e.g., recipient upon administration). Provided are methods of treating a disorder comprising repeat dosing of a population of hypoimmunogenic cells to a recipient subject in need thereof.

In some embodiments, the pluripotent stem cell and any cell differentiated from such a pluripotent stem cell is modified to exhibit reduced expression of MHC class I human leukocyte antigens. In other embodiments, the pluripotent stem cell and any cell differentiated from such a pluripotent stem cell is modified to exhibit reduced expression of MHC class II human leukocyte antigens. In certain embodiments, the pluripotent stem cell and any cell differentiated from such a pluripotent stem cell is modified to exhibit reduced expression of TCR complexes. In some embodiments, the pluripotent stem cell and any cell differentiated from such a pluripotent stem cell is modified to exhibit reduced expression of MHC class I and II human leukocyte antigens. In some embodiments, the pluripotent stem cell and any cell differentiated from such a pluripotent stem cell is modified to exhibit reduced expression of MHC class I and II human leukocyte antigens and TCR complexes.

In some embodiments, the pluripotent stem cell and any cell differentiated from such a pluripotent stem cell is modified to exhibit reduced expression of MHC class I and/or II human leukocyte antigens and exhibit increased CD47 expression. In some instances, the cell overexpresses CD47 by harboring one or more CD47 transgenes. In some embodiments, the pluripotent stem cell and any cell differentiated from such a pluripotent stem cell is modified to exhibit reduced expression of MHC class I and II human leukocyte antigens and exhibit increased CD47 expression. In some embodiments, the pluripotent stem cell and any cell differentiated from such a pluripotent stem cell is modified to exhibit reduced expression of MHC class I and II human leukocyte antigens and TCR complexes and exhibit increased CD47 expression.

In some embodiments, the pluripotent stem cell and any cell differentiated from such a pluripotent stem cell is modified to exhibit reduced expression of MHC class I and/or II human leukocyte antigens, to exhibit increased CD47 expression, and to exogenously express a chimeric antigen receptor. In some instances, the cell overexpresses CD47 polypeptides by harboring one or more CD47 transgenes. In some instances, the cell overexpresses CAR polypeptides by harboring one or more CAR transgenes. In some embodiments, the pluripotent stem cell and any cell differentiated from such a pluripotent stem cell is modified to exhibit reduced expression of MHC class I and II human leukocyte antigens, exhibit increased CD47 expression, and to exogenously express a chimeric antigen receptor. In some embodiments, the pluripotent stem cell and any cell differentiated from such a pluripotent stem cell is modified to exhibit reduced expression of MHC class I and II human leukocyte antigens and TCR complexes, to exhibit increased CD47 expression, and to exogenously express a chimeric antigen receptor.

Such pluripotent stem cells are hypoimmunogenic stem cells. Such differentiated cells are hypoimmunogenic cells.

Any of the pluripotent stem cells described herein can be differentiated into any cells of an organism and tissue. In some embodiments, the cells exhibit reduced expression of MHC class I and/or II human leukocyte antigens and reduced expression of TCR complexes. In some instances, expression of MHC class I and/or II human leukocyte antigens is reduced compared to unmodified or wild-type cell of the same cell type. In some instances, expression of TCR complexes is reduced compared to unmodified or wild-type cell of the same cell type. In some embodiments, the cells exhibit increased CD47 expression. In some instances, expression of CD47 is increased in cells encompassed by the present disclosure as compared to unmodified or wild-type cells of the same cell type. In some embodiments, the cells exhibit exogenous CAR expression. Methods for reducing levels of MHC class I and/or II human leukocyte antigens and TCR complexes and increasing the expression of CD47 and CARs are described herein.

In some embodiments, the cells used in the methods described herein evade immune recognition and responses when administered to a patient (e.g., recipient subject). The cells can evade killing by immune cells in vitro and in vivo. In some embodiments, the cells evade killing by macrophages and NK cells. In some embodiments, the cells are ignored by immune cells or a subject's immune system. In other words, the cells administered in accordance with the methods described herein are not detectable by immune cells of the immune system. In some embodiments, the cells are cloaked and therefore avoid immune rejection.

Methods of determining whether a pluripotent stem cell and any cell differentiated from such a pluripotent stem cell evades immune recognition include, but are not limited to, IFN-γ Elispot assays, microglia killing assays, cell engraftment animal models, cytokine release assays, ELISAs, killing assays using bioluminescence imaging or chromium release assay or a real-time, quantitative microelectronic biosensor system for cell analysis (xCELLigence® RTCA system, Agilent), mixed-lymphocyte reactions, immunofluorescence analysis, etc.

Therapeutic cells outlined herein are useful to treat a disorder such as, but not limited to, a cancer, a genetic disorder, a chronic infectious disease, an autoimmune disorder, a neurological disorder, and the like.

1. T Lymphocytes Differentiated from Hypoimmunogenic Pluripotent Cells

Provided herein, T lymphocytes (T cells, including primary T cells) are derived from the HIP cells described herein (e.g., hypoimmunogenic iPSCs). Methods for generating T cells, including CAR-T cells, from pluripotent stem cells (e.g., iPSCs) are described, for example, in Iriguchi et al., Nature Communications 12, 430 (2021); Themeli et al., Cell Stem Cell, 16(4):357-366 (2015); Themeli et al., Nature Biotechnology 31:928-933 (2013).

T lymphocyte derived hypoimmunogenic cells include, but are not limited to, primary T cells that evade immune recognition. In some embodiments, the hypoimmunogenic cells are produced (e.g., generated, cultured, or derived) from T cells such as primary T cells. In some instances, primary T cells are obtained (e.g., harvested, extracted, removed, or taken) from a subject or an individual. In some embodiments, primary T cells are produced from a pool of T cells such that the T cells are from one or more subjects (e.g., one or more human including one or more healthy humans). In some embodiments, the pool of primary T cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects. In some embodiments, the donor subject is different from the patient (e.g., the recipient that is administered the therapeutic cells). In some embodiments, the pool of T cells does not include cells from the patient. In some embodiments, one or more of the donor subjects from which the pool of T cells is obtained are different from the patient.

In some embodiments, the hypoimmunogenic cells do not activate an immune response in the patient (e.g., recipient upon administration). Provided are methods of treating a disorder by administering a population of hypoimmunogenic cells to a subject (e.g., recipient) or patient in need thereof. In some embodiments, the hypoimmunogenic cells described herein comprise T cells engineered (e.g., are modified) to express a chimeric antigen receptor including but not limited to a chimeric antigen receptor described herein. In some instances, the T cells are populations or subpopulations of primary T cells from one or more individuals. In some embodiments, the T cells described herein such as the engineered or modified T cells comprise reduced expression of an endogenous T cell receptor.

In some embodiments, the HIP-derived T cell includes a chimeric antigen receptor (CAR). Any suitable CAR can be included in the hyHIP-derived T cell, including the CARs described herein. In some embodiments, the hypoimmunogenic induced pluripotent stem cell-derived T cell includes a polynucleotide encoding a CAR, wherein the polynucleotide is inserted in a genomic locus. In some embodiments, the polynucleotide is inserted into a safe harbor or target locus. In some embodiments, the polynucleotide is inserted in a B2M, CIITA, TRAC, TRB, PD-1 or CTLA-4 gene. Any suitable method can be used to insert the CAR into the genomic locus of the hypoimmunogenic cell including the gene editing methods described herein (e.g., a CRISPR/Cas system).

HIP-derived T cells provided herein are useful for the treatment of suitable cancers including, but not limited to, B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.

2. NK Cells Derived from Hypoimmunogenic Pluripotent Cells

Provided herein, natural killer (NK) cells are derived from the HIP cells described herein (e.g., hypoimmunogenic iPSCs).

NK cells (also defined as ‘large granular lymphocytes’) represent a cell lineage differentiated from the common lymphoid progenitor (which also gives rise to B lymphocytes and T lymphocytes). Unlike T-cells, NK cells do not naturally comprise CD3 at the plasma membrane. Importantly, NK cells do not express a TCR and typically also lack other antigen-specific cell surface receptors (as well as TCRs and CD3, they also do not express immunoglobulin B-cell receptors, and instead typically express CD16 and CD56). NK cell cytotoxic activity does not require sensitization but is enhanced by activation with a variety of cytokines including IL-2. NK cells are generally thought to lack appropriate or complete signaling pathways necessary for antigen-receptor-mediated signaling, and thus are not thought to be capable of antigen receptor-dependent signaling, activation and expansion. NK cells are cytotoxic, and balance activating and inhibitory receptor signaling to modulate their cytotoxic activity. For instance, NK cells expressing CD16 may bind to the Fc domain of antibodies bound to an infected cell, resulting in NK cell activation. By contrast, activity is reduced against cells expressing high levels of MHC class I proteins. On contact with a target cell NK cells release proteins such as perforin, and enzymes such as proteases (granzymes). Perforin can form pores in the cell membrane of a target cell, inducing apoptosis or cell lysis.

There are a number of techniques that can be used to generate NK cells, including CAR-NK-cells, from pluripotent stem cells (e.g., iPSC); see, for example, Zhu et al., Methods Mol Biol. 2019; 2048:107-119; Knorr et al., Stem Cells Transl Med. 2013 2(4):274-83. doi: 10.5966/sctm.2012-0084: Zeng et al., Stem Cell Reports. 2017 Dec. 12; 9(6):1796-1812: Ni et al., Methods Mol Biol. 2013:1029:33-41; Bemareggi et al., Exp Hematol. 2019 71:13-23; Shankar et al., Stem Cell Res Ther. 2020;11(1):234, all of which are incorporated herein by reference in their entirety and specifically for the methodologies and reagents for differentiation. Differentiation can be assayed as is known in the art, generally by evaluating the presence of NK cell associated and/or specific markers, including, but not limited to, CD56, KIRs, CD16, NKp44, NKp46, NKG2D, TRAIL, CD122, CD27, CD244, NK1.1, NKG2A/C, NCR1, Ly49, CD49b, CD11b, KLRG1, CD43, CD62L, and/or CD226.

In some embodiments, the hypoimmunogenic pluripotent cells are differentiated into hepatocytes to address loss of the hepatocyte functioning or cirrhosis of the liver. There are a number of techniques that can be used to differentiate HIP cells into hepatocytes; see for example, Pettinato et al., doi: 10.1038/spre32888, Snykers et al., Methods Mol Biol., 2011 698:305-314, Si-Tayeb et al., Hepatology, 2010, 51:297-305 and Asgari et al., Stem Cell Rev., 2013, 9(4):493-504, all of which are incorporated herein by reference in their entirety and specifically for the methodologies and reagents for differentiation. Differentiation can be assayed as is known in the art, generally by evaluating the presence of hepatocyte associated and/or specific markers, including, but not limited to, albumin, alpha fetoprotein, and fibrinogen. Differentiation can also be measured functionally, such as the metabolization of ammonia, LDL storage and uptake, ICG uptake and release, and glycogen storage.

In some embodiments, the NK cells do not activate an innate and/or an adaptive immune response in the patient (e.g., recipient upon administration). Provided are methods of treating a disorder by administering a population of NK cells to a subject (e.g., recipient) or patient in need thereof. In some embodiments, the NK cells described herein comprise NK cells engineered (e.g., are modified) to express a chimeric antigen receptor including but not limited to a chimeric antigen receptor described herein. Any suitable CAR can be included in the NK cells, including the CARs described herein. In some embodiments, the NK cell includes a polynucleotide encoding a CAR, wherein the polynucleotide is inserted in a genomic locus. In some embodiments, the polynucleotide is inserted into a safe harbor or a target locus. In some embodiments, the polynucleotide is inserted in a B2M, CIITA. PD1 or CTLA4 gene. Any suitable method can be used to insert the CAR into the genomic locus of the NK cell including the gene editing methods described herein (e.g., a CRISPR/Cas system).

### G. Assays for Hypoimmunogenicity Phenotypes and Retention of Pluripotency

Once the hypoimmunogenic cells have been generated, they may be assayed for their hypoimmunogenicity and/or retention of pluripotency as is described in WO2016183041 and WO2018132783.

In some embodiments, hypoimmunogenicity is assayed using a number of techniques as exemplified in FIG. 13 and FIG. 15 of WO2018132783. These techniques include transplantation into allogeneic hosts and monitoring for hypoimmunogenic pluripotent cell growth (e.g., teratomas) that escape the host immune system. In some instances, hypoimmunogenic pluripotent cell derivatives are transduced to express luciferase and can then followed using bioluminescence imaging. Similarly, the T cell and/or B cell response of the host animal to such cells are tested to confirm that the cells do not cause an immune reaction in the host animal. T cell responses can be assessed by Elispot. ELISA, FACS, PCR, or mass cytometry (CYTOF). B cell responses or antibody responses are assessed using FACS or Luminex. Additionally, or alternatively, the cells may be assayed for their ability to avoid innate immune responses, e.g., NK cell killing, as is generally shown in FIGS. 14 and 15 of WO2018132783.

In some embodiments, the immunogenicity of the cells is evaluated using T cell immunoassays such as T cell proliferation assays, T cell activation assays, and T cell killing assays recognized by those skilled in the art. In some cases, the T cell proliferation assay includes pretreating the cells with interferon-gamma and coculturing the cells with labelled T cells and assaying the presence of the T cell population (or the proliferating T cell population) after a preselected amount of time. In some cases, the T cell activation assay includes coculturing T cells with the cells outlined herein and determining the expression levels of T cell activation markers in the T cells.

In vivo assays can be performed to assess the immunogenicity of the cells outlined herein. In some embodiments, the survival and immunogenicity of hypoimmunogenic cells is determined using an allogenic humanized immunodeficient mouse model. In some instances, the hypoimmunogenic pluripotent stem cells are transplanted into an allogenic humanized NSG-SGM3 mouse and assayed for cell rejection, cell survival, and teratoma formation. In some instances, grafted hypoimmunogenic pluripotent stem cells or differentiated cells thereof display long-term survival in the mouse model.

Additional techniques for determining immunogenicity including hypoimmunogenicity of the cells are described in, for example, Deuse et al., Nature Biotechnology, 2019, 37, 252-258 and Han et al., Proc Natl Acad Sci USA, 2019, 116(21), 10441-10446, the disclosures including the figures, figure legends, and description of methods are incorporated herein by reference in their entirety.

Similarly, the retention of pluripotency is tested in a number of ways. In some embodiments, pluripotency is assayed by the expression of certain pluripotency-specific factors as generally described herein and shown in FIG. 29 of WO2018132783. Additionally or alternatively, the pluripotent cells are differentiated into one or more cell types as an indication of pluripotency.

As will be appreciated by those in the art, the successful reduction of the MHC I function (HLA I when the cells are derived from human cells) in the pluripotent cells can be measured using techniques known in the art and as described below; for example, FACS techniques using labeled antibodies that bind the HLA complex; for example, using commercially available HLA-A, HLA-B, and HLA-C antibodies that bind to the alpha chain of the human major histocompatibility HLA Class I antigens.

In addition, the cells can be tested to confirm that the HLA I complex is not expressed on the cell surface. This may be assayed by FACS analysis using antibodies to one or more HLA cell surface components as discussed above.

The successful reduction of the MHC II function (HLA II when the cells are derived from human cells) in the pluripotent cells or their derivatives can be measured using techniques known in the art such as Western blotting using antibodies to the protein. FACS techniques, RT-PCR techniques, etc.

In addition, the cells can be tested to confirm that the HLA II complex is not expressed on the cell surface. Again, this assay is done as is known in the art (See FIG. 21 of WO2018132783, for example) and generally is done using either Western Blots or FACS analysis based on commercial antibodies that bind to human HLA Class II HLA-DR, DP and most DQ antigens.

In addition to the reduction of HLA I and II (or MHC I and II), the hypoimmunogenic cells of the technology have a reduced susceptibility to macrophage phagocytosis and NK cell killing. The resulting hypoimmunogenic cells “escape” the immune macrophage and innate pathways due to reduction or lack of the TCR complex and the expression of one or more CD47 transgenes.

In some aspects, the present technology provides T cells, such as immune evasive allogeneic T cells, that are derived from or generated by methods according to various embodiments disclosed herein. In some embodiments, the generated T cells are suitable for use in adoptive cell therapy, as they have been made to be immune evasive (e.g., by inserting a tolerogenic factor into an endogenous TCR gene locus and/or by modifying the MHC I and/or MHC II genes as described) and to express one or more CARs.

In some embodiments, the T cell is a naïve T cell, a helper T cell (CD4+), a cytotoxic T cell (CD8+), a regulatory T cell (Treg), a central memory T cell (T_{CM}), an effector memory T cell (T_{EM}), a stem cell memory T cell (T_{SCM}), or any combination thereof. More specifically, the T cell can be naïve (not exposed to antigen; increased expression of CD62L, CCR7, CD28, CD3, CD127, and CD45RA, and decreased expression of CD45RO as compared to T_{CM}), memory T cells (antigen-experienced and long-lived), or effector cells (antigen-experienced, cytotoxic). Memory T cells can be further divided into subsets of Tcm (increased expression of CD62L, CCR7, CD28, CD127, CD45RO, and CD95, and decreased expression of CD54RA as compared to naïve T cells) and T_{EM} (decreased expression of CD62L, CCR7, CD28, CD45RA, and increased expression of CD127 as compared to naïve T cells or T_{CM}). Effector T cells refer to antigen-experienced CD8+ cytotoxic T cells that has decreased expression of CD62L, CCR7, CD28, and are positive for granzyme and perforin as compared to Tcm. Helper T cells are CD4+ cells that influence the activity of other immune cells by releasing cytokines. CD4+ T cells can activate or suppress an adaptive immune response, and which of those two functions is induced will depend on the presence of other cells and signals. T cells can be collected using known techniques, and the various subpopulations or combinations thereof can be enriched or depleted by known techniques, such as by affinity binding to antibodies, flow cytometry, or immunomagnetic selection.

In some embodiments, the T cell is an autologous cell, i.e., obtained from the subject who will receive the T cell after modification. In some embodiments, the T cell is an allogeneic T cell. i.e., obtained from someone other than the subject who will receive the T cell after modification. In either of these embodiments, the T cells can be primary T cells obtained from a number of sources, including peripheral blood mononuclear cells, bone marrow, lymph node tissue, cord blood, thymus tissue, tissue from a site of infection, ascites, pleural effusion, spleen tissue, and tumors. In other embodiments, especially in the case of allogeneic T cells, the T cells can be derived or differentiated from embryonic stem cells (ESCs) or induced pluripotent cells (iPSCs).

In some aspects, the present technology provides pharmaceutical compositions comprising a T cell according to various embodiments disclosed herein.

In some embodiments, the compositions can have various formulations, for example, injectable formulations, lyophilized formulations, liquid formulations, oral formulations, etc., depending on the suitable routes of administration.

In some embodiments, the compositions can be co-formulated in the same dosage unit or can be individually formulated in separate dosage units. The terms “dose unit” and “dosage unit” herein refer to a portion of a pharmaceutical composition that contains an amount of a therapeutic agent suitable for a single administration to provide a therapeutic effect. Such dosage units may be administered one to a plurality (i.e., 1 to 10, 1 to 8, 1 to 6, 1 to 4, or 1 to 2) of times per day, or as many times as needed to elicit a therapeutic response.

In some embodiments, a single dosage unit includes at least about 1×10², 5×10², 1×10³, 5×10³, 1×10⁴, 5×10⁴, 1×10⁵, 5×10⁵, 1×10⁶, 5×10⁶, 1×1×10⁷, 5×10⁷, 1 ×10⁸, 5×10⁸, 1×10⁹, 5×10⁹, 1×10¹⁰, or 5×10¹⁰ cells.

In some embodiments, the provided engineered cells are modified such that they are able to evade immune recognition and responses when administered to a patient (e.g., recipient subject). The cells can evade killing by immune cells in vitro and in vivo. In some embodiments, the cells evade killing by macrophages and NK cells. In some embodiments, the cells are ignored by immune cells or a subject's immune system. In other words, the cells administered in accordance with the methods described herein are not detectable by immune cells of the immune system. In some embodiments, the cells are cloaked and therefore avoid immune rejection.

Methods of determining whether an engineered cell provided herein evades immune recognition include, but are not limited to, IFN-γ Elispot assays, microglia killing assays, cell engraftment animal models, cytokine release assays, ELISAs, killing assays using bioluminescence imaging or chromium release assay or Xcelligence analysis, mixed-lymphocyte reactions, immunofluorescence analysis, etc.

In some embodiments, the immunogenicity of the cells is evaluated in a complement-dependent cytotoxicity (CDC) assay. CDC can be assayed in vitro by incubating cells with IgG or IgM antibodies targeting an HLA-independent antigen expressed on the cell surface in the presence of serum containing complement and analyzing cell killing. In some embodiments, CDC can be assayed by incubating cells with ABO blood type incompatible serum, wherein the cells comprise A antigens or B antigens, and the serum comprises antibodies against the A antigens and/or B antigens of the cells.

In some embodiments, once the engineered cells have been modified or generated as described herein, they may be assayed for their hypoimmunogenicity. Any of a variety of assays can be used to assess if the cells are hypoimmunogenic or can evade the immune system. Exemplary assays include any as is described in WO2016183041 and WO2018132783. In some embodiments, the engineered cells described herein survive in a host without stimulating the host immune response for one week or more (e.g., one week, two weeks, one month, two months, three months, 6 months, one year, two years, three years, four years, five years or more, e.g., for the life of the cell and/or its progeny). The cells maintain expression of the transgenes and/or are deleted or reduced in expression of target genes for as long as they survive in the host. In some aspects, if the transgenes are no longer expressed and/or if target genes are expressed the engineered cells may be removed by the host's immune system. In some embodiments, the persistence or survival of the engineered cells may be monitored after their administration to a recipient by further expressing a transgene encoding a protein that allows the cells to be detected in vivo (e.g., a fluorescent protein, such as GFP, a truncated receptor or other surrogate marker or other detectable marker).

The hypoimmunogenic cells are administered in a manner that permits them to engraft to the intended tissue site and reconstitute or regenerate the functionally deficient area. In some embodiments, the hypoimmunogenic cells are assayed for engraftment (e.g., successful engraftment). In some embodiments, the engraftment of the hypoimmunogenic cells is evaluated after a pre-selected amount of time. In some embodiments, the engrafted cells are monitored for cell survival. For example, the cell survival may be monitored via bioluminescence imaging (BLI), wherein the cells are transduced with a luciferase expression construct for monitoring cell survival. In some embodiments, the engrafted cells are visualized by immunostaining and imaging methods known in the art. In some embodiments, the engrafted cells express known biomarkers that may be detected to determine successful engraftment. For example, flow cytometry may be used to determine the surface expression of particular biomarkers. In some embodiments, the hypoimmunogenic cells are engrafted to the intended tissue site as expected (e.g., successful engraftment of the hypoimmunogenic cells). In some embodiments, the hypoimmunogenic cells are engrafted to the intended tissue site as needed, such as at a site of cellular deficiency. In some embodiments, the hypoimmunogenic cells are engrafted to the intended tissue site in the same manner as a cell of the same type not comprising the modifications.

In some embodiments, the hypoimmunogenic cells are assayed for function. In some embodiments, the hypoimmunogenic cells are assayed for function prior to their engraftment to the intended tissue site. In some embodiments, the hypoimmunogenic cells are assayed for function following engraftment to the intended tissue site. In some embodiments, the function of the hypoimmunogenic cells is evaluated after a pre-selected amount. In some embodiments, the function of the engrafted cells is evaluated by the ability of the cells to produce a detectable phenotype. For example, engrafted beta islet cells function may be evaluated based on the restoration of lost glucose control due to diabetes. In some embodiments, the function of the hypoimmunogenic cells is as expected (e.g., successful function of the hypoimmunogenic cells while avoiding antibody-mediated rejection). In some embodiments, the function of the hypoimmunogenic cells is as needed, such as sufficient function at a site of cellular deficiency while avoiding antibody-mediated rejection. In some embodiments, the engineered cells function in the same manner as a non-engineered cell of the same type.

It will be understood that embodiments concerning HIP cells may be readily applied to any cell type as described herein, as well as combined with safety switches, CAR modification and other modification/gene edit as described herein.

### H. Safety Switches

As disclosed above, in some embodiments, a safety switch can be incorporated into, such as introduced, into the engineered cells provided herein to provide the ability to induce death or apoptosis of engineered cells containing the safety switch, for example if the cells grow and divide in an undesired manner or cause excessive toxicity to the host. Thus, the use of safety switches enables one to conditionally eliminate aberrant cells in vivo and can be a critical step for the application of cell therapies in the clinic. Relevant information concerning safety switches as referred to in the context of the present disclosure may be found from WO2021/146627, the contents of which are herein incorporated by reference. It will be understood that embodiments concerning safety switches may be readily applied to any cell type as described herein, as well as combined with embodiments relating to HIP cells, CAR modification and other modification/gene edit as described herein. The following definitions apply to the present disclosure:

The term “safety switch” used herein refers to a system for controlling the expression of a gene or protein of interest that, when downregulated or upregulated, leads to clearance or death of the cell, e.g., through recognition by the host's immune system. A safety switch can be designed to be triggered by an exogenous molecule in case of an adverse clinical event. A safety switch can be engineered by regulating the expression on the DNA, RNA and protein levels. A safety switch includes a protein or molecule that allows for the control of cellular activity in response to an adverse event. In some embodiments, the safety switch is a ‘kill switch’ that is expressed in an inactive state and is fatal to a cell expressing the safety switch upon activation of the switch by a selective, externally provided agent. In some embodiments, the safety switch gene is cis-acting in relation to the gene of interest in a construct. In some embodiments, the safety switch is an “uncloaking” system wherein upon activation, cells downregulate expression of immunosuppressive factors and/or upregulate expression of immune signaling molecules thereby marking the cell for elimination by the host immune system. Activation of the safety switch causes the cell to kill solely itself or itself and neighboring cells through apoptosis or necrosis, or causes the cell to be killed by the host immune system.

By “HLA” or “human leukocyte antigen” complex is a gene complex encoding the major histocompatibility complex (MHC) proteins in humans. These cell-surface proteins that make up the HLA complex are responsible for the regulation of the immune response to antigens. In humans, there are two MHCs, class I and class II, “HLA-I” and “HLA-II”. HLA-I includes three proteins, HLA-A, HLA-B and HLA-C, which present peptides from the inside of the cell, and antigens presented by the HLA-I complex attract killer T cells (also known as CD8+ T cells or cytotoxic T cells). The HLA-I proteins are associated with β-2 microglobulin (B2M). HLA-II includes five proteins, HLA-DP, HLA-DM, HLA-DOB, HLA-DQ and HLA-DR, which present antigens from outside the cell to T lymphocytes. This stimulates CD4+ T cells (also known as helper T cells). It should be understood that the use of either “MHC” or “HLA” is not meant to be limiting, as it depends on whether the genes are from humans (HLA) or murine (MHC). Thus, as it relates to mammalian cells, these terms may be used interchangeably herein.

### 1. Immune Signaling Gene Locus

Provided herein is an isolated cell or a population thereof comprising a construct described. In some embodiments, the construct has been introduced into a target gene locus. In some embodiments, the gene locus is either a safe harbor locus selected from the group consisting of an AAVS1 locus, a CLBYL locus, a CXCR4 locus, a Rosa26 locus, and a CCR5 locus, or an immune signaling gene locus selected from the group consisting of B2M. HLA-A, HLA-B. HLA-C, HLA-D, HLA-E, RFXANK, CIITA, CTLA-4, PD-1, RAETE/ULBP4, RAET1G/ULBP5, RAET1H/ULBP2, RAET1/ULBP1, RAET1ILULBP6, RAET1N/ULBP3, and other ligands of NKG2D. In some embodiments, the isolated cell is an isolated engineered human cell further comprising deletion or reduced expression of MHC class I human leukocyte antigens and/or deletion or reduced expression of MHC class II human leukocyte antigens compared to an unmodified human cell. In some embodiments, the isolated cell further comprises deletion or reduced expression of CIITA. B2M, and/or NLRC5. In some embodiments, the isolated cell is hypoimmunogenic and is a stem cell.

In some embodiments, the immune signaling gene locus is selected from the group consisting of an B2M, HLA-A, HLA-B, HLA-C, HLA-D, HLA-E, RFXANK, CIITA, CTLA-4, PD-1, RAET1FE/ULBP4, RAET1G/ULBP5, RAET1H/ULBP2, RAET1/ULBP1, RAE11L/ULBP6, RAET1N/ULBP3, and other ligands of NKG2D.

In some embodiments, the immune signaling gene locus is selected from the group consisting of B2M, HLA-A, HLA-B, HLA-C, HLA-D, and HLA-E.

In some aspects, reduced or eliminated expression of CIITA reduces or eliminates expression of one or more of the following MHC class II are HLA-DP, HLA-DM, HLA-DOA, HLA-DOB. HLA-DQ, and HLA-DR.

### 2. Conditional HIP Cells and Methods for Conditional Downregulation of Immunosuppressive Factors

The introduction of safety switches improves the safety of cell therapies developed using hypoimmunogenic cells (HIP cells). A feature of the HIP cells described herein is the inducible expression of one or more immune regulatory (immunosuppressive) factors. In some embodiments, an immunosuppressive factor (also referred to herein as “an hypoimmunity factor” or “a tolerogenic factor”) includes, but is not limited to, CD47, CD24, CD200, HLA-G, HLA-E, HLA-C. HLA-E heavy chain, PD-L1, IDO1, CTLA4-Ig, C1-Inhibitor, IL-10, IL-35, FASL, Serpinb9, CC121, and Mfge8. In certain embodiments, the immunosuppressive factor is CD47. The regulatable or inducible expression of an immunosuppressive factor functions to control an immune response by a recipient subject to an engrafted hypoimmunogenic cell.

Described herein are methods for the expression of an immunosuppressive factor that requires a mechanism to ‘turn-off’ expression of the immune regulatory protein in a controlled manner. Also described are HIP cells possessing controllable expression of one or more immunosuppressive factors. In some cases, the cells overexpress one or more immunosuppressive factors and can be induced to downregulate expression of the one or more immunosuppressive factors. As such, the cells are no longer hypoimmunogenic and are recognized by the recipient's immune cells for cell death. In some embodiments, the hypoimmunity of the cells that are introduced to a recipient subject is achieved through the overexpression of an immunosuppressive molecule including hypoimmunity factors and complement inhibitors accompanied with the repression or genetic disruption of the HLA-I and HLA-II loci. These modifications cloak the cell from the recipient immune system's effector cells that are responsible for the clearance of infected, malignant or non-self cells, such as T cells. B cells, NK cells and macrophages. Cloaking of a cell from the immune system allows for existence and persistence of allogeneic cells within the body. Controlled removal of the engineered cells from the body is crucial for patient safety and can be achieved by uncloaking the cells from the immune system. Uncloaking serves as a safety switch and can be achieved through the downregulation of the immunosuppressive molecules or the upregulation of immune signaling molecules. The level of expression of any of the immunosuppressive molecules described can be controlled on the protein level, mRNA level, or DNA level in the cells. Similarly, the level of expression of any of the immune signaling molecules described can be controlled on the protein level, mRNA level, or DNA level in the cells.

In some embodiments, any of the safety switch methods described (e.g., protein level, RNA level and DNA level safety switches) are used to decrease the level of an immunosuppressive factor in the cells such that the lower level of the immunosuppressive factor is below a threshold level. In some embodiments, the level of the immunosuppressive factor in the cells is decreased by about 10-fold, 9-fold, 8-fold, 7-fold, 6-fold, 5-fold, 4-fold, 3-fold, 2-fold, 1-fold or 0.5-fold below a threshold level of expression. In some embodiments, the level of the immunosuppressive factor in the cells is decreased by about 10-fold to 5-fold, 10-fold to 3-fold, 9-fold to 1-fold, 8-fold to 1-fold, 7-fold to 0.5-fold, 6-fold, to 1-fold, 5-fold to 0.5-fold, 4-fold to 0.5-fold, 3-fold to 0.5-fold, 2-fold to 0.5-fold, or 1-fold to 0.5-fold below a threshold level of expression. In some embodiments, the threshold level of expression of the immunosuppressive factor is established based on the expression of such factor in an induced pluripotent stem cell. In some embodiments, the threshold level of the immunosuppressive factor expression is established based on the expression level of the immunosuppressive factor in a corresponding hypoimmune cell, such as an MHC I and MHC II knockout cell or an MHC I/MHC II/TCR knockout cell.

### 3. Protein Level Control

In some embodiments, regulated degradation of an immunosuppressive protein is established by incorporating a degron into the amino acid sequence of the immunosuppressive factor that allows recruitment to the endogenous protein turnover machinery. Mechanisms for targeted protein degradation include, but are not limited to, recruitment to an E3 ligase for ubiquitination and subsequent proteasomal degradation, direct recruitment to the proteasome, and recruitment to the lysosome.

Fusion of inducible degron motifs to the immunosuppressive molecules enables exogenous control over the stability of the molecule through the addition or removal of small molecules that stabilize or destabilize the degron, and thus the immunosuppressive molecule.

In some embodiments, methods for inducible protein degradation by a degron includes, but is not limited to, ligand induced degradation (LID) using a SMASH tag, ligand induced degradation using Shield-1, ligand induced degradation using auxin, ligand induced degradation using rapamycin, peptidic degrons (e.g., IKZF3 based degrons), and proteolysis-targeting chimeras (PROTACs). In some embodiments of a ligand induced degradation method, a degron tag that is held in an inactive conformation but is induced to adopt a conformation capable of recognition by the proteasome upon binding of a specific molecule, such as but not limited to, a Shield-1 molecule. See, e.g., Roth et al., Cellular Molecular Life Sciences, 2019, 76(14), 2761-2777, which is herein incorporated by reference in its entirety. Detailed descriptions of SMASH degron technology can be found in Hannah and Zhou, Nat Chem Biol, 2015, 11:637-638 and Chung et al., Nat Chem Biol, 2015, 11:713-720, which are herein incorporated by reference in their entireties. Detailed descriptions of LID degron technologies can be found in Bonger et al., Nat Chem Biol. 2011, 7(8):531-7, which is herein incorporated by reference in its entirety.

In some aspects, provided are methods for controlling the immunogenicity of a mammalian cell (e.g., a human cell) by obtaining an isolated cell and introducing a construct containing a constitutive promoter operably linked to an inducible degron element that is operably linked to a gene encoding an immunosuppressive factor. In some embodiments, the construct includes a constitutive promoter operably linked to an inducible degron element that is operably linked to a nucleic acid sequence encoding flexible linker that is operable linked to agene encoding an immunosuppressive factor. In some embodiments, the construct comprising a constitutive promoter operably linked to a gene encoding an immunosuppressive factor that is operably linked to an inducible degron element. In some embodiments, the construct includes a constitutive promoter operably linked to a gene encoding an immunosuppressive factor that is linked to a sequence encoding a flexible linker that is operably linked to an inducible degron element. As such, the degron targets the immunosuppressive factor for degradation upon contacting the cell with a degron ligand or molecule.

In some embodiments, the inducible degron element is selected from the group consisting of a ligand inducible degron element such as a small molecule-assisted shutoff (SMASH) degron element, Shield-1 responsive degron element, auxin responsive degron element, and rapamycin responsive degron element: a peptidic degron element; and a peptidic proteolysis targeting chimera (PROTAC) element. In useful embodiments, the ligand inducible degron element is a small molecule-assisted shutoff (SMASH) degron element and the exogenous factor for controlling immunogenicity is asunaprevir. In some embodiments, the immunosuppressive factor gene is selected from the group consisting of CD47, CD24. CD200, HLA-G, HLA-E, HLA-C, HLA-E heavy chain, PD-L1, IDO1, CTLA4-Ig, C1-Inhibitor, IL-10, IL-35, FASL, Serpinb9, CC121, and Mfge8. In many embodiments, the immunosuppressive factor gene is CD47. In some instances, the constitutive promoter of the construct is selected from the group consisting of an EF1A promoter, an EFS promoter, a CMV promoter, a CAGGS promoter, a SV40 promoter, a COPIA promoter, an ACT5C promoter, a TRE promoter, a CBh promoter, a PGK promoter, and a UBC promoter. In some instances, the optional flexible linker is selected from the group consisting of (GSG)n (SEQ ID NO:3), (GGGS)n (SEQ ID NO:1), and (GGGSGGGS)n (SEQ ID NO:2), wherein n is 1-10. In some embodiments, the construct is introduced into the cell to integrated into a safe harbor locus, such as but not limited to, an AAVS1 locus, a CLBYL locus, a CXCR4 locus, a Rosa26 locus, and a CCR5 locus. In some embodiments, the construct is introduced into the AAVS locus in the cell by way for homology directed recombination. As such, the construct includes 5′ and 3′ homology arms specific to the targeted safe harbor locus. In some embodiments, the construct comprises from 5′ end to 3′ end: a 5′ homology arm to the AAVS1 locus, an exogenous constitutive promoter, an inducible degron element, a gene encoding an immunosuppressive factor, and a 3′ homology arm to the AAVS1 locus. In other embodiments, the construct comprises from 5′ end to 3′ end: a 5′ homology arm to the AAVS1 locus, an exogenous constitutive promoter, an inducible degron element, a sequence encoding flexible linker, a gene encoding an immunosuppressive factor, and a 3′ homology arm to the AAVS1 locus. In useful embodiments, the engineered cell includes an exogenous nucleic acid sequence comprising a constitutive promoter operably linked to an inducible degron element that is operably linked to an optional sequence encoding a flexible linker that is operable linked to a gene encoding an immunosuppressive factor. The engineered cell expresses the inducible degron element fused or linked to an immunosuppressive factor. In some embodiments, the cell is contacted by a factor or agent such as, but not limited to, a ligand, molecule, peptide or small molecule, that activates the degron element to degrade the immunosuppressive factor.

In some embodiments of a peptidic degron, a peptide tag is used that confers small molecule-mediated recruitment to an E3 ligase. In some embodiments, the peptide tag comprises the lymphoid-restricted transcription factor IKZF3 that is recruited to the E3 ligase receptor (CRBN) in an immunomodulatory drug (IMiD) dependent manner, as described in Koduri et al., Proc Natl Acad Sci, 2019, 116(7), 2539-2544, which is herein incorporated by reference in its entirety. In certain embodiments, the degron is capable of targeting immunosuppressive factors for degradation (e.g., through a ubiquitination pathway), inducing protein degradation, or degrading proteins.

In some aspects, provided are methods for controlling the immunogenicity of a mammalian cell (e.g., a human cell) by obtaining an isolated cell and introducing a construct including a constitutive promoter, an inducible peptidic degron element, and a gene encoding an immunosuppressive factor. In some embodiments, the construct includes a constitutive promoter, an inducible peptidic degron element, a nucleic acid sequence encoding flexible linker, and a gene encoding an immunosuppressive factor. Any of the constitutive promoters, immunosuppressive factors, flexible linkers, and cells described herein are applicable to the method.

In some embodiments of a PROTAC, a bifunctional molecule is used to recruit an immunosuppressive factor to the protein degradation machinery of a cell. In some embodiments, the bi-functional molecule binds to the native or wildtype sequence of the immunosuppressive protein or an engineered version of the immunosuppressive protein expressing a domain that binds to the bi-functional molecule with high affinity. In some embodiments, the bi-functional molecule comprises a small molecule or a biologic agent (e.g., an antibody or fragment thereof). See, e.g., Burslem et al., Cell Chemical Biology, 2018, 25, 67-77 and Roth et al., Cellular Molecular Life Sciences, 2019, 76(14), 2761-2777, which are herein incorporated by reference in their entirety.

In some embodiments of a bi-functional antibody, the antibody targets an immunosuppressive factor and a second endogenous receptor which leads to internalization and degradation. Controllable expression of one or more immunosuppressive factors can be provided by way of a bifunctional antibody (e.g., a chemically reprogrammed bifunctional antibody), inducible protein degradation by a degron, inducible RNA regulation, inducible DNA regulation, and an inducible expression method. See, e.g., Natsume and Kanemaki, Annu Rev Genet, 2017, 51.82-102; Burslem and Crews, Chem Rev, 2017, 117, 11269-11301; Banik et al., ChemRxiv, 2019; which are herein incorporated by reference in their entirety. In some embodiments, a cell expressing an immunosuppressive factor is contacted by an antibody that binds the cell for degradation.

In some instances, hypoimmune cells are availed and cleared by the immune system through the addition of an antibody that binds an epitope on the extracellular surface of the cell. The epitope can be native to the overexpressed immunosuppressive factor, or can be another epitope located within the immunosuppressive factor or distinctly located at the extracellular surface. Binding of an antibody to the surface uncloaks the cell and leads to antibody-dependent cellular cytotoxicity (ADCC) or complement-dependent cytotoxicity (CDC).

In some embodiments, the ADCC/CDC safety switch epitope is selected from the group consisting of EGFR, CD20, CD19, CCR4, HER2, MUC1, GD2, PSMA, CD30, CD16, and fragment, derivative, and variants thereof. In some instances, any of the cells described herein express an epitope selected from an EGFR epitope, CD20 epitope, CD19 epitope, CCR4 epitope, HER2 epitope, MUC1 epitope, GD2 epitope, PSMA epitope, CD30 epitope, or CD16 epitope. In some embodiments, the cells bind to an antibody specific to EGFR, CD20, CD19, CCR4, HER2, MUC1, GD2, PSMA, CD30, or CD16, which leads to ADCC/CDC.

The methods directed to a protein level safety switch as described herein provides a way for decreasing the level of an immunosuppressive factor (e.g., CD47) in an regulatable manner in engineered cells described herein (e.g., hypoimmune cells). By lowering the level of the immunosuppressive factor such as CD47 below a threshold level in the cells using any of the safety switch methods described herein, the recipient subject's immune system can initiate an immune response to such cells. In some embodiments, the level of CD47 in the engineered cells is decreased by the safety switch by about 10-fold, 9-fold, 8-fold, 7-fold, 6-fold, 5-fold, 4-fold, 3-fold, 2-fold, 1-fold or 0.5-fold below a threshold level of expression. In some embodiments, the level of CD47 in the engineered cells is decreased by about 10-fold to 5-fold, 10-fold to 3-fold, 9-fold to 1-fold, 8-fold to 1-fold, 7-fold to 0.5-fold, 6-fold, to 1-fold, 5-fold to 0.5-fold, 4-fold to 0.5-fold, 3-fold to 0.5-fold, 2-fold to 0.5-fold, or 1-fold to 0.5-fold below a threshold level of expression. In some instances, the threshold level of CD47 expression is established based on the exogenous expression of CD47 in an induced pluripotent stem cell. In other instances, the threshold level of CD47 expression is established based on the expression level of CD47 in a corresponding hypoimmune cell, such as an MHC I and MHC II knockout cell or an MHC I/MHC II/TCR knockout cell. In some instances, the level of CD47 is reduced using a degron-based safety switch such as, but not limited to, a SMASH degron or a LID degron. In some embodiments, the cells expressing a SMASH degron linked to an exogenous CD47 transgene are exposed to the small molecule asunaprevir (the degron inducer), which thereby induces a reduction of expression of the exogenous CD47 by the cells.

### 4. RNA Level Control

Immunosuppressive factors can be targeted by siRNAs or miRNAs, thereby leading to the degradation of the transcript encoding the factors. An siRNA can be exogenously provided or genetically encoded to provide control over transcription of the inhibitory RNA. The siRNA or miRNA can anneal to the immunosuppressive factor's transcript, resulting in degradation by the RISC complex

In some embodiments, methods for inducible RNA regulation to downregulate expression of an immunosuppressive factor include, but are not limited to, shRNAs induced by a small molecule or a biologic agent, inducible siRNAs, inducible miRNAs, inducible CRISPR interference (CRISPRi), and inducible RNA targeting nucleases.

In some embodiments, the method comprises an shRNA or siRNA targeting the RNA of the immunosuppressive factor. In some instances, expression of the shRNA or siRNA is induced by a small molecule or biologic agent.

In some aspects, provided are methods for controlling the immunogenicity of a mammalian cell (e.g., a human cell) by obtaining an isolated cell and introducing a construct containing an inducible RNA polymerase promoter operably linked an shRNA sequence targeting an immunosuppressive factor that is operably linked to a constitutive promoter that is operably linked to a transactivator element that can control the inducible RNA polymerase promoter. In some embodiments, the construct includes a U6Tet promoter, an shRNA targeting an immunosuppressive factor, a constitutive promoter, and a Tet Repressor element that is responsive to tetracycline or a derivative thereof (e.g., doxycycline). In other instances, the shRNA eliminates expression of the immunosuppressive factor. In other instances, the shRNA decreases expression of the immunosuppressive factor by about 99% or less, e.g., 99%, 98%, 97%, 96%, 95%, 94%, 93%, 92%, 90%, 85% or less. In some embodiments, the inducible promoter is a tetracycline responsive promoter. Any of the constitutive promoters, immunosuppressive factors, and cells described herein are applicable to the method.

In many embodiments, the engineered cell expresses an inducible shRNA that targets an immunosuppressive factor. In some embodiments, the cell also expresses an exogenous immunosuppressive factor that mediates the hypoimmunogenicity of the cell. In some embodiments, the cell is contacted by a factor such as, but not limited to, a ligand, molecule, peptide or small molecule, that activates the expression of the shRNA to degrade the immunosuppressive factor.

In some embodiments, the method comprises a CRISPR interference system (CRISPRi) for targeting the promoter of an immunosuppressive factor to downregulate its transcription. In some instances, expression of a CRISPRi and/or a gRNA targeting the immunosuppressive factor is induced by a small molecule or biologic agent. Detailed description of CRISPRi methods are found in, e.g., Engreitz et al., Cold Spring Harb Perspect Biol, 2019, 11:a035386, which is herein incorporated by reference in its entirety. In some embodiments, the CRISPRi system utilizes a dCas9-repressor fusion protein that is controlled by a constitutive promoter and a gRNA specific to the immunosuppressive factor under the control of an inducible promoter.

In some aspects, provided are methods for controlling the immunogenicity of a mammalian cell (e.g., a human cell) by obtaining an isolated cell and introducing into the cell (i) a first construct containing a constitutive promoter operably linked to a gene encoding an immunosuppressive factor; (ii) a second construct containing a constitutive promoter operably linked to a gene encoding a Cas9 nuclease or variant thereof such as dCas9-repressor fusion protein; and (iii) a third construct comprising an inducible RNA polymerase promoter operably linked to a gRNA sequence targeting the sequence encoding the immunosuppressive factor such that the gRNA sequence is operably linked to a transactivator element that corresponds to the inducible RNA polymerase promoter. In some instances, the first construct, second construct, and third construct are found in a single vector. In some instances, the first construct, second construct, and third construct are found in two vectors.

In some embodiments, the CRISPR based method includes a nuclease for targeting the mRNA sequence corresponding to the immunosuppressive factor such as, but not limited to, Cas13, Cas7, or Csx1. In some instances, expression of a nuclease and/or a gRNA targeting the immunosuppressive factor is induced by a small molecule or biologic agent.

In some aspects, provided are methods for controlling the immunogenicity of a mammalian cell (e.g., a human cell) by obtaining an isolated cell and introducing into the cell (i) a first construct comprising a constitutive promoter operably linked to a gene encoding an immunosuppressive factor: (ii) a second construct comprising a constitutive promoter operably linked to a gene encoding a Cas13a nuclease, a variant thereof, or a fusion protein thereof; and (iii) a third construct comprising an inducible RNA polymerase promoter operably linked to a gRNA sequence targeting the sequence encoding the immunosuppressive factor such that the gRNA sequence is operably linked to a transactivator element that corresponds to the inducible RNA polymerase promoter.

In some embodiments, inducible expression systems that are useful for RNA level control of the immunosuppressive factor include, but are not limited to, ligand inducible transcription factor systems, receptor mediated expression control systems, and ligand regulated riboswitches. In some embodiments, the inducible expression system comprises a tetracycline-controlled operator system, a synthetic Notch-based (SynNotch) system (see, e.g., Morsut et al., Cell, 2016, 164:780-791 and Yang et al., Commun Biol, 2020, 3:116), and riboswitch that regulates expression of the immunosuppressive factor gene by ligand (e.g., aptamer, peptide or small molecule) mediated alternative splicing of the resulting pre-mRNA. Useful riboswitches comprise a sensor region and an effector region that sense the presence of a ligand and alter the splice of the target immunosuppressive factor gene. Detailed descriptions and examples of riboswitch gRNAs are found in e.g., U.S. Pat. Nos. 9,228,207; 9,993,491; and 10,421,989; and Seeliger et al., PLoS One, 2012, 7(1):e29266; the contents are herein incorporated by reference in their entirety.

In some embodiments, the level of an immunosuppressive factor such as CD47 in the engineered cells is decreased by an RNA level safety switch by about 10-fold, 9-fold, 8-fold, 7-fold, 6-fold, 5-fold, 4-fold, 3-fold, 2-fold, 1-fold or 0.5-fold below a threshold level of expression. In some embodiments, the level of CD47 in the engineered cells is decreased by about 10-fold to 5-fold, 10-fold to 3-fold, 9-fold to 1-fold, 8-fold to 1-fold, 7-fold to 0.5-fold, 6-fold, to 1-fold, 5-fold to 0.5-fold, 4-fold to 0.5-fold, 3-fold to 0.5-fold, 2-fold to 0.5-fold, or 1-fold to 0.5-fold below a threshold level of expression. In some instances, the threshold level of CD47 expression is established based on the exogenous expression of CD47 in an induced pluripotent stem cell. In other instances, the threshold level of CD47 expression is established based on the expression level of CD47 in a corresponding hypoimmune cell, such as an MHC I and MHC II knockout cell or an MHC I/MHC II/TCR knockout cell.

### 5. DNA Level Control

Transcriptional regulation of immunosuppressive factors through employing inducible promoters provides the ability to turn expression of the switch on or off at will through the addition or removal of small molecules, such as, but not limited to, doxycycline. Genetic disruption via targeted nuclease activity can eliminate expression of the immunosuppressive factor to uncloak the cells as well.

In some embodiments, methods for inducible DNA regulation include, but are not limited to, using tissue-specific promoters, inducible promoters, controllable riboswitches, and knockout using an inducible nuclease (e.g., inducible CRISPRs, inducible TALENs, inducible zinc finger nucleases, inducible homing endonucleases, inducible meganucleases, and the like) to target the DNA sequence of one or more immunosuppressive factors. In some embodiments, the inducible nuclease comprises a nuclease such that its expression is controlled by the presence of a small molecule. In some embodiments, the inducible nuclease comprises a nuclease such that delivery of the nuclease RNA or protein to a cells is controlled by the presence of a small molecule. In some embodiments, expression of the nuclease is induced by a small molecule or biologic agent. In some embodiments, expression of a Cas nuclease and/or a guide RNA (gRNA) is induced by a small molecule or biologic agent.

In some embodiments, methods for inducible expression include, but are not limited to, ligand inducible transcription factors systems (e.g., a tetracycline-controlled operator system), receptor mediated control of expression system (e.g., a SynNotch system), and a ligand regulated riboswitch system for control of mRNA or gRNA activity. Detailed description of inducible expression methods are found in, e.g., Kallunki et al., Cells, 2019, 796 (doi:10.3390/cells8080796), which is herein incorporated by reference in its entirety.

In some embodiments, the immunosuppressive factors are expressed in a cell using an inducible expression vector. The expression vector can be a viral vector, such as but not limited to, a lentiviral vector. In some embodiments, the inducible immunosuppressive factors described herein are introduced into a cell by lentiviral transduction.

In some embodiments, the silencing of a construct encoding the immunosuppressive factor results in elimination of the engineered cell by a recipient subject's immune system. Furthermore, the construct containing the immunosuppressive factor and an inducible expression system can be integrated into an endogenous gene locus to safeguard expression of the cassette, as silencing of the gene will eliminate the engineered cells. In some embodiments, the endogenous gene locus useful for integration is a core essential gene locus or an immune signaling factor gene locus. Non-limiting examples of a core essential gene locus for such integration include RpS2, RpS9, RpS 11, RpS13, RpS18, RpL8, RpL11, RpL32, RpL36, Rpn11, Psmd14, and PSMA3. Non-limiting examples of an immune signaling factor gene locus for such integration include B2M, MIC-AB, HLA-A, HLA-B, HLA-C, RFXANK, CTLA4, PD1, and ligands of NKG2D (e.g., MICA, MICB, RAET1E/ULBP4, RAET1G/ULBP5, RAET1H/ULBP2, RAET1/ULBP1, RAET1L/ULBP6, and RAET1N/ULBP3).

In some embodiments, the conditional expression of an immunosuppressive factor is based on regulating expression of the immune regulatory factor CD47. CD47 is a component of the innate immune system that functions as a “do not eat me” signal as part of the innate immune system to block phagocytosis by macrophages. Useful immunosuppressive factors that can be engineered for controlled expression include, but are not limited to, CD47, CD27, CD200, HLA-C, HLA-E, HLA-E heavy chain, HLA-G, PD-L1, IDO1, CTLA4-Ig, C1-Inhibitor, IL-10, IL-35, FASL, Serpinb9, CCL21, and Mfge8.

In some embodiments, the present disclosure provides a method of producing a stem cell (e.g., hypoimmunogenic pluripotent stem cell or hypoimmunogenic induced pluripotent stem cell) or a differentiated cell thereof that has been modified to conditionally express any one of the immunosuppressive factors selected from the group consisting of CD47, CD27, CD200, HLA-C, HLA-E, HLA-E heavy chain, HLA-G, PD-L 1, IDO1, CTLA4-Ig, C1-Inhibitor, IL-10, IL-35, FASL, Serpinb9, CCL21, and Mfge8. In other embodiments, the immunosuppressive factor is selected from the group consisting of HLA-A. HLA-B, HLA-C, RFX-ANK. CIITA, NFY-A, NLRC5, B2M. RFX5, RFX-AP, HLA-G, HLA-E, NFY-B. PD-L1, NFY-C, IRF1, TAP1, GITR, 4-1BB, CD28, B7-1, CD47, B7-2, OX40, CD27, HVEM, SLAM, CD226, ICOS, LAG3, TIGIT, TIM3, CD160, BTLA, CD244, LFA-1, ST2, HLA-F, CD30, B7-H3, VISTA, TLT, PD-L2, CD58, CD2, and HELIOS.

In some embodiments, the cells conditionally express one or more of the immunosuppressive factors such that in the absence of the exogenous controlling signal, the cells are hypoimmunogenic or have reduced hypoimmunogenicity. In the presence of the exogenous controlling signal, the cells are recognized by immune cells and are targeted by cell death or clearance. In some instances, the HIP cells express an immunosuppressive factor that functions allow the HIP cell to evade the recipient subject's immune response. Upon exposing the HIP cells to an exogenous controlling signal, the expression (e.g., the DNA level expression, the RNA level expression, or the protein level expression) of immunosuppressive factor is downregulated, and thus the HIP cells are recognized by the innate immune system in the recipient subject. As such, the HIP cells undergo cell death and/or cell clearance in the recipient.

In some embodiments, the level of an immunosuppressive factor such as CD47 in the engineered cells is decreased by a DNA level safety switch by about 10-fold, 9-fold, 8-fold, 7-fold, 6-fold, 5-fold, 4-fold, 3-fold, 2-fold, 1-fold or 0.5-fold below a threshold level of expression. In some embodiments, the level of CD47 in the engineered cells is decreased by about 10-fold to 5-fold, 10-fold to 3-fold, 9-fold to 1-fold, 8-fold to 1-fold, 7-fold to 0.5-fold, 6-fold, to 1-fold, 5-fold to 0.5-fold, 4-fold to 0.5-fold, 3-fold to 0.5-fold, 2-fold to 0.5-fold, or 1-fold to 0.5-fold below a threshold level of expression. In some instances, the threshold level of CD47 expression is established based on the exogenous expression of CD47 in an induced pluripotent stem cell. In other instances, the threshold level of CD47 expression is established based on the expression level of CD47 in a corresponding hypoimmune cell, such as an MHC I and MHC II knockout cell or an MHC I/MHC II/TCR knockout cell.

### 6. Conditional HIP Cells and Methods Conditional Upregulation of Immune Signaling Factors

Described herein are methods for the expression of an immune signaling factor in a controllable manner as to increase the expression of the factor to alter the hypoimmunogenicity of the cell. Also described are HIP cells that possess controllable expression of one or more immune signaling factors. In some aspects, the immune signaling factor is selected from the group consisting of B2M, MIC-A/B, HLA-A, HLA-B. HLA-C, RFXANK, CTLA-4, PD-1, and ligands of NKG2D (e.g., MICA, MICB, RAET1E/ULBP4, RAET1G/ULBP5, RAET1H/ULBP2, RAET1/ULBP1, RAET1L/ULBP6, and RAET1N/ULBP3).

Controllable expression of one or more immune signaling factors can be provided by way of a inducible ligand stabilization system using a degron, an inducible RNA upregulation system (e.g., an inducible CRISPR activation), and an inducible DNA upregulation system. In some embodiments, the inducible DNA upregulation system comprises inducible CRISPR activation (CRISPRa), tissue-specific promoters, inducible promoters, and riboswitches.

Detailed description of CRISPRa methods are found in, e.g., Engreitz et al., Cold Spring Harb Perspect Biol, 2019, 11:a035386, which is herein incorporated by reference in its entirety. Detailed descriptions and examples of inducible riboswitches are found in e.g., U.S. Pat. No. 9,228,207: U.S. Pat. Nos. 9,993,491; and 10,421,989; and Seeliger et al., PLoS One, 2012, 7(1):e29266; the contents are herein incorporated by reference in their entirety.

In an example of uncloaking Hypo-Immune cells Through Genetic, Post-Transcriptional, and Post-Translational Regulation, hypoimmunity is achieved through the overexpression of hypoimmune molecules such as CD47, complement inhibitors accompanied with the repression or genetic disruption of the HLA-1 and HLA-II loci. These modifications cloak the cell from the immune system's effector cells that are responsible for the clearance of infected, malignant or non-self cells, such as T-cells, B-cells, NK cells and macrophages. Cloaking of a cell from the immune system allows for existence and persistence of allogeneic cells within the body. Removal of the engineered cells from the body is crucial for patient safety and can be achieved by uncloaking the cells from the immune system. Uncloaking serves as a safety switch and can be achieved through the downregulation of the hypoimmune molecules (for example CD47, CD27, CD200, HLA-C, HLA-E, HLA-E heavy chain, HLA-G, PD-L1, IDO1, CTLA4-Ig, C1-Inhibitor, IL-10, IL-35, FASL, Serpinb9, CCL21, or Mfge8) or the upregulation of immune signaling molecules (for example B2M, MIC-A/B, HLA-A, HLA-B, HLA-C, RFXANK, CTLA-4, PD-1, and ligands of NKG2D (e.g., MICA, MICB, RAET1E/ULBP4, RAET1G/ULBP5, RAET1H/ULBP2, RAET1/ULBP1, RAET1L/ULBP6, or RAET1N/ULBP3). Either of these activities will avail the cell to native effector cells, resulting in clearance of the allogeneic cell.

### II. Populations of Engineered Cells and Pharmaceutical Compositions

As described elsewhere herein, in the process of manufacturing a cell therapy, certain modifications may be introduced to the cell. Provided herein are populations of cells containing a plurality of the provided engineered cells. Provided herein are also populations of cells containing a plurality of engineered cells.

In the ‘end’ population of cells, i.e., the population of cells being used in the cell therapy product (pharmaceutical composition), unedited or partially edited cells may be viewed as contaminants likely to adversely affect the function of the cell therapy product. In some circumstances, the percentage of edited cells may be viewed as a measure of purity with respect to the proportion of edited versus unedited or partially edited cells in a population. This is particularly in relation to hypoimmune gene modifications that enable immune evasion where the presence of cells not having the hypoimmune gene modifications might be expected to adversely affect the in vivo efficacy of the cell therapy product. Surprisingly, as has been demonstrated by the present inventors, engineered cells within a population of cells may be functional even if the population of cells contains unedited or partially edited cells. Accordingly, a novel cell therapy product comprising a population of cells is hereby provided, wherein the population of cells contains some unedited or partially edited cells.

In some embodiments, up to 20%, up to 30%, up to 40%, up to 50%, up to 60%, or up to 65% of cells in the population are not HIP modified cells (e.g., do not exhibit reduced expression of one or more molecules of the MHC class I and/or MHC class II molecules and optionally also do not exhibit increased expression of at least one tolerogenic factor).

In some embodiments, up to 20%, up to 30%, up to 40%, up to 50%, up to 60%, or up to 65% of cells in the cell therapy product do not exhibit reduced expression of one or more molecules of the MHC class I and/or MHC class II molecules. In some embodiments, up to 20%, up to 30%, up to 40%, up to 50%, up to 60%, or up to 65% of cells in the cell therapy product (i) do not exhibit reduced expression of one or more molecules of the MHC class I and/or MHC class II molecules and (ii) do not exhibit increased expression of at least one tolerogenic factor. In either of these embodiments, the proportion of cells in the cell therapy product that express a CAR may be in the range 70-100%, 80-100%, 90-100%. In some embodiments, at least 10%, 20%, 30%, 40%, 50%, 60%, or 70% of cells in the cell therapy product do not exhibit reduced expression of one or more molecules of the MHC class I and/or MHC class II molecules. In some embodiments, at least 10%, 20%, 30%, 40%, 50%, 60%, or 70% of cells in the cell therapy product (i) do not exhibit reduced expression of one or more molecules of the MHC class I and/or MHC class II molecules and (ii) do not exhibit increased expression of at least one tolerogenic factor. In either of these embodiments, the proportion of cells in the cell therapy product that express a CAR may be in the range 70-100%, 80-100%, 90-100%.

In some embodiments, at least 30%, 40%, 50%, 60%, 70%, 80%, 90%, 95%, 96%, 97%, 98%, 99%, 99.9%, or 99.99% of cells in the population comprise a set of modifications described herein. In some embodiments, the set of modifications reduce expression of one or more molecules of the MHC class I and/or MHC class II molecules and increase expression of at least one tolerogenic factor, such as tolerogenic factors described herein. In some embodiments, the set of modifications reduce expression of one or more molecules of the MHC class I and/or MHC class II molecules and increase expression of at least one tolerogenic factor, such as tolerogenic factors described herein and incorporate a CAR transgene. In some embodiments, the set of modifications reduce expression of one or more molecules of the MHC class I and/or MHC class II molecules and incorporate a CAR transgene.

In some embodiments at least 30%, 40%, 50%, 60%, 70%, 80%, 90%, 95%, 96%, 97%, 98%, 99%, 99.9%, or 99.99% of cells in the population comprise a set of modifications that reduce expression of one or more MHC class I molecules and/or one or more MHC class II molecules, and that increase expression of one or more tolerogenic factor. In some embodiments, the one or more tolerogenic factor is one or more of CD16. CD24, CD35, CD39, CD46, CD47, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, MANF, or any combination thereof. In some embodiments, the one or more tolerogenic factor is CD47. In some embodiments, at least 30%, 40%, 50%, 60%, 70%, 80%, 90%, 95%, 96%, 970%, 98%, 99%, 99.9%, or 99.99% of cells in the population comprise an exogenous polynucleotide encoding CD47.

In some embodiments, at least 30%, 40%, 50%, 60%, 70%, 80%, 90%, 95%, 96%, 97%, 98%, 99%, 99.9%, or 99.99% of cells in the population comprise one or more alterations that inactivate both alleles of a B2M gene. In some embodiments, at least 30%, 40%, 50%, 60%, 70%, 80%, 90%, 95%, 96%, 97%, 98%, 99%, 99.9%, or 99.99% of cells in the population comprise one or more alterations that inactivate both alleles of a CHTA gene.

In some embodiments, up to 40% of cells in the cell therapy product do not exhibit reduced expression of one or more molecules of the MHC class I and/or MHC class II molecules and the proportion of cells in the cell therapy product that express a CAR is in the range 70-100%, 80-100%, 90-100%. In some embodiments, up to 50% of cells in the cell therapy product do not exhibit reduced expression of one or more molecules of the MHC class I and/or MHC class II molecules and the proportion of cells in the cell therapy product that express a CAR is in the range 70-100%, 80-100%, 90-100%. In some embodiments, up to 60% of cells in the cell therapy product do not exhibit reduced expression of one or more molecules of the MHC class I and/or MHC class II molecules and the proportion of cells in the cell therapy product that express a CAR is in the range 70-100%, 80-100%, 90-100%.

In some embodiments, at least 40% of cells in the cell therapy product (i) do not exhibit reduced expression of one or more molecules of the MHC class I and/or MHC class II molecules and (ii) do not exhibit increased expression of at least one tolerogenic factor and the proportion of cells in the cell therapy product that express a CAR is in the range 70-100%, 80-100%, 90-100%. In some embodiments, at least 50% of cells in the cell therapy product (i) do not exhibit reduced expression of one or more molecules of the MHC class I and/or MHC class II molecules and (ii) do not exhibit increased expression of at least one tolerogenic factor and the proportion of cells in the cell therapy product that express a CAR is in the range 70-100%, 80-100%, 90-100%. In some embodiments, at least 60% of cells in the cell therapy product (i) do not exhibit reduced expression of one or more molecules of the MHC class I and/or MHC class II molecules and (ii) do not exhibit increased expression of at least one tolerogenic factor and the proportion of cells in the cell therapy product that express a CAR is in the range 70-100%, 80-100%, 90-100%.

In some embodiments, the proportion of cells in the population that comprise the set of modifications as described herein is 30-90%, 30-80%, 30-70%, 30-60%, 30-50% or 40-50%. In some embodiments, the proportion of cells in the population that comprise the set of modifications as described herein is 40-90%, 40-80%, 40-70%, 40-60% or 40-50%. In some embodiments, the proportion of cells in the population that comprise the set of modifications as described herein is 50-90%, 50-80%, 50-70% or 50-60%.

In some embodiments, 30-90/a, 30-80%, 30-70%, 30-60%, 30-50% or 40-50% of cells in the cell therapy product are HIP modified cells (e.g., exhibiting reduced expression of one or more molecules of the MHC class I and/or MHC class II molecules and optionally increased expression of at least one tolerogenic factor). In some embodiments, 40-90%, 40-80%, 40-70%, 40-60% or 40-50% of cells in the cell therapy product are HIP modified cells (e.g., exhibiting reduced expression of one or more molecules of the MHC class I and/or MHC class II molecules and optionally increased expression of at least one tolerogenic factor). In some embodiments, 50-90%, 50-80%, 50-70% or 50-60% of cells in the cell therapy product are HIP modified cells (e.g., exhibiting reduced expression of one or more molecules of the MHC class I and/or MHC class II molecules and optionally increased expression of at least one tolerogenic factor). In any of these embodiments, the proportion of cells in the cell therapy product that express a CAR may be in the range 70-100%, 80-100%, 90-100%.

In some embodiments, 30-80% of cells in the cell therapy product are HIP modified cells exhibiting reduced expression of one or more molecules of the MHC class I and/or MHC class II molecules and 80-100% of the cells express a CAR In some embodiments, 30-70% of cells in the cell therapy product are HIP modified cells exhibiting reduced expression of one or more molecules of the MHC class I and/or MHC class II molecules and 80-100% of the cells express a CAR. In some embodiments, 30-60% of cells in the cell therapy product are HIP modified cells exhibiting reduced expression of one or more molecules of the MHC class I and/or MHC class II molecules and 80-100% of the cells express a CAR. In some embodiments. 30-50% of cells in the cell therapy product are HIP modified cells exhibiting reduced expression of one or more molecules of the MHC class I and/or MHC class II molecules and 80-100% of the cells express a CAR

In some embodiments. 30-80% of cells in the cell therapy product are HIP modified cells exhibiting reduced expression of one or more molecules of the MHC class I and/or MHC class II molecules and increased expression of at least one tolerogenic factor and 90-100% of the cells express a CAR. In some embodiments, 30-70% of cells in the cell therapy product are HIP modified cells exhibiting reduced expression of one or more molecules of the MHC class I and/or MHC class II molecules and increased expression of at least one tolerogenic factor and 90-100% of the cells express a CAR. In some embodiments, 30-60% of cells in the cell therapy product are HIP modified cells exhibiting reduced expression of one or more molecules of the MHC class I and/or MHC class II molecules and increased expression of at least one tolerogenic factor and 90-100% of the cells express a CAR In some embodiments. 30-50% of cells in the cell therapy product are HIP modified cells exhibiting reduced expression of one or more molecules of the MHC class I and/or MHC class II molecules and increased expression of at least one tolerogenic factor and 90-100% of the cells express a CAR.

In some embodiments, 30-80% of cells in the cell therapy product are HIP modified cells exhibiting reduced expression of one or more molecules of the MHC class I and/or MHC class II molecules and increased expression of at least one tolerogenic factor and 80-100% of the cells express a CAR In some embodiments. 30-70% of cells in the cell therapy product are HIP modified cells exhibiting reduced expression of one or more molecules of the MHC class I and/or MHC class II molecules and increased expression of at least one tolerogenic factor and 80-100% of the cells express a CAR In some embodiments, 30-60% of cells in the cell therapy product are HIP modified cells exhibiting reduced expression of one or more molecules of the MHC class I and/or MHC class II molecules and increased expression of at least one tolerogenic factor and 80-100% of the cells express a CAR In some embodiments, 30-50% of cells in the cell therapy product are HIP modified cells exhibiting reduced expression of one or more molecules of the MHC class I and/or MHC class II molecules and increased expression of at least one tolerogenic factor and 80-100% of the cells express a CAR.

In some embodiments, 30-80% of cells in the cell therapy product are HIP modified cells exhibiting reduced expression of one or more molecules of the MHC class I and/or MHC class II molecules and increased expression of at least one tolerogenic factor and 90-100% of the cells express a CAR. In some embodiments, 30-70% of cells in the cell therapy product are HIP modified cells exhibiting reduced expression of one or more molecules of the MHC class I and/or MHC class II molecules and increased expression of at least one tolerogenic factor and 90-100% of the cells express a CAR In some embodiments. 30-60% of cells in the cell therapy product are HIP modified cells exhibiting reduced expression of one or more molecules of the MHC class I and/or MHC class II molecules and increased expression of at least one tolerogenic factor and 90-100% of the cells express a CAR In some embodiments, 30-50% of cells in the cell therapy product are HIP modified cells exhibiting reduced expression of one or more molecules of the MHC class I and/or MHC class II molecules and increased expression of at least one tolerogenic factor and 90-100% of the cells express a CAR.

Also provided herein are compositions comprising the engineered cells or populations of engineered cells. In some embodiments, the compositions are pharmaceutical compositions.

In some embodiments, the pharmaceutical composition provided herein further include a pharmaceutically acceptable excipient or carrier. Acceptable carriers, excipients, or stabilizers are nontoxic to recipients at the dosages and concentrations employed, and include buffers such as phosphate, citrate, and other organic acids; antioxidants including ascorbic acid and methionine: preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben: catechol; resorcinol; cyclohexanol; 3-pentanol; and m-cresol); low molecular weight (less than about 10 residues) polypeptides: proteins, such as serum albumin, gelatin, or immunoglobulins: hydrophilic polymers such as polyvinylpyrrolidone: amino acids such as glycine, glutamine, asparagine, histidine, arginine, or lysine; monosaccharides, disaccharides, and other carbohydrates including glucose, mannose, or dextrins; chelating agents such as EDTA: sugars such as sucrose, mannitol, trehalose or sorbitol; salt-forming counter-ions such as sodium; metal complexes (e.g., Zn-protein complexes); and/or non-ionic surfactants such as polysorbates (TWEEN™), poloxamers (PLURONICS™) or polyethylene glycol (PEG). In some embodiments, the pharmaceutical composition includes a pharmaceutically acceptable buffer (e.g., neutral buffer saline or phosphate buffered saline). In some embodiments, the pharmaceutical composition can contain one or more excipients for modifying, maintaining or preserving, for example, the pH, osmolarity, viscosity, clarity, color, isotonicity, odor, sterility, stability, rate of dissolution or release, adsorption, or penetration of the composition. In some aspects, a skilled artisan understands that a pharmaceutical composition containing cells may differ from a pharmaceutical composition containing a protein.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The pharmaceutical composition in some embodiments contains engineered cells as described herein in amounts effective to treat or prevent the disease or condition, such as a therapeutically effective or prophylactically effective amount. In some embodiments, the pharmaceutical composition contains engineered cells as described herein in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In some embodiments, engineered cells as described herein are administered using standard administration techniques, formulations, and/or devices. In some embodiments, engineered cells as described herein are administered using standard administration techniques, formulations, and/or devices. Provided are formulations and devices, such as syringes and vials, for storage and administration of the compositions. Engineered cells can be administered via localized injection, including catheter administration, systemic injection, localized injection, intravenous injection, or parenteral administration. When administering a therapeutic composition (e.g., a pharmaceutical composition containing an engineered cell), it will generally be formulated in a unit dosage injectable form (solution, suspension, emulsion).

Formulations include those for intravenous, intraperitoneal, or subcutaneous, administration. In some embodiments, the cell populations are administered parenterally. The term “parenteral.” as used herein, includes intravenous, intramuscular, subcutaneous, rectal, vaginal, and intraperitoneal administration. In some embodiments, the cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, or dispersions, which may in some aspects be buffered to a selected pH. Liquid compositions are somewhat more convenient to administer, especially by injection. Liquid compositions can comprise carriers, which can be a solvent or dispersing medium containing, for example, water, saline, phosphate buffered saline, polyol (for example, glycerol, propylene glycol, liquid polyethylene glycol) and suitable mixtures thereof. Sterile injectable solutions can be prepared by incorporating the cells in a solvent, such as in admixture with a suitable carrier, diluent, or excipient such as sterile water, physiological saline, glucose, dextrose, or the like.

In some embodiments, a pharmaceutically acceptable carrier can include all solvents, dispersion media, coatings, antibacterial and antifungal agents, isotonic and absorption delaying agents, and the like, compatible with pharmaceutical administration (Gennaro, 2000, Remington: The science and practice of pharmacy. Lippincott, Williams & Wilkins, Philadelphia, PA). Examples of such carriers or diluents include, but are not limited to, water, saline, Ringer's solutions, dextrose solution, and 5% human serum albumin. Liposomes and non-aqueous vehicles such as fixed oils may also be used. Supplementary active compounds can also be incorporated into the compositions. The pharmaceutical carrier should be one that is suitable for the engineered cells, such as a saline solution, a dextrose solution or a solution comprising human serum albumin. In some embodiments, the pharmaceutically acceptable carrier or vehicle for such compositions is any non-toxic aqueous solution in which the engineered cells can be maintained, or remain viable, for a time sufficient to allow administration of live cells. For example, the pharmaceutically acceptable carrier or vehicle can be a saline solution or buffered saline solution.

In some embodiments, the composition, including pharmaceutical composition, is sterile. In some embodiments, isolation, enrichment, or culturing of the cells is carried out in a closed or sterile environment, for example and for instance in a sterile culture bag, to minimize error, user handling and/or contamination. In some embodiments, sterility may be readily accomplished, e.g., by filtration through sterile filtration membranes. In some embodiments, culturing is carried out using a gas permeable culture vessel. In some embodiments, culturing is carried out using a bioreactor.

In some embodiments, the cells and compositions provided herein may be stored. In some embodiments, the cells and compositions provided herein may be stored for 1-72 hours. In some embodiments, the cells and compositions provided herein may be stored for 1-7 days. In some embodiments, the cells and compositions provided herein may be stored for 1-5 weeks. In some embodiments, the cells and compositions provided herein may be stored for 1-12 months. In some embodiments, the cells and compositions provided herein may be stored for 1-30 years.

In some embodiments, the cells and compositions provided herein may be stored after they have been collected from a donor or pool of donors. In some embodiments, the cells and compositions provided herein may be stored before manufacturing. In some embodiments, the cells and compositions provided herein may be stored after starting manufacturing. In some embodiments, the cells and compositions provided herein may be stored after completing 1, 2, 3, 4, 5, 6, 7, 8, 9, 11, 12, 13, 14, 15, 16, 17, 18, 19, or 20 steps of the manufacturing process. In some embodiments, the cells and compositions provided herein may be stored after completing 1 or more steps of the manufacturing process. In some embodiments, the cells and compositions provided herein may be stored after completing the manufacturing process. In some embodiments, the cells and compositions provided herein may be stored before modification. In some embodiments, the cells and compositions provided herein may be stored after starting modification. In some embodiments, the cells and compositions provided herein may be stored after completing 1, 2, 3, 4, 5, 6, 7, 8, 9, 11, 12, 13, 14, 15, 16, 17, 18, 19, or 20 modifications. In some embodiments, the cells and compositions provided herein may be stored after completing 1 or more modifications. In some embodiments, the cells and compositions provided herein may be stored after completing modification. In some embodiments, the cells and compositions provided herein may be stored before gene-editing. In some embodiments, the cells and compositions provided herein may be stored after starting gene-editing. In some embodiments, the cells and compositions provided herein may be stored after completing 1, 2, 3, 4, 5, 6, 7, 8, 9, 11, 12, 13, 14, 15, 16, 17, 18, 19, or 20 gene-edits. In some embodiments, the cells and compositions provided herein may be stored after completing 1 or more gene-edits. In some embodiments, the cells and compositions provided herein may be stored after completing gene-editing. In some embodiments, the cells and compositions provided herein may be stored before viral transduction. In some embodiments, the cells and compositions provided herein may be stored after starting viral transduction. In some embodiments, the cells and compositions provided herein may be stored after completing 1, 2, 3, 4, 5, 6, 7, 8, 9, 11, 12, 13, 14, 15, 16, 17, 18, 19, or 20 viral transductions. In some embodiments, the cells and compositions provided herein may be stored after completing 1 or more viral transductions. In some embodiments, the cells and compositions provided herein may be stored after completing viral transduction.

In some embodiments, the cells and compositions may be stored in liquid nitrogen. In some embodiments, the cells and compositions may be stored in a freezer at −80° C. In some embodiments, the cells and compositions may be stored in a freezer at −20° C. In some embodiments, the cells and compositions may be stored on ice. In some embodiments, the cells and compositions may be stored on dry ice. In some embodiments, the cells and compositions may be stored in refrigerator at 4° C.

Also provided herein are compositions that are suitable for cryopreserving the provided engineered cells. In some embodiments, the provided engineered cells are cryopreserved in a cryopreservation medium. In some embodiments, the cryopreservation medium is a serum free cryopreservation medium. In some embodiments, the composition comprises a cryoprotectant. In some embodiments, the cryoprotectant is or comprises DMSO and/or s glycerol. In some embodiments, the cryopreservation medium is between at or about 5% and at or about 10% DMSO (v/v). In some embodiments, the cryopreservation medium is at or about 5% DMSO (v/v). In some embodiments, the cryopreservation medium is at or about 6% DMSO (v/v). In some embodiments, the cryopreservation medium is at or about 7% DMSO (v/v). In some embodiments, the cryopreservation medium is at or about 7.5% DMSO (v/v). In some embodiments, the cryopreservation medium is at or about 8% DMSO (v/v). In some embodiments, the cryopreservation medium is at or about 9% DMSO (v/v). In some embodiments, the cryopreservation medium is at or about 10% DMSO (v/v). In some embodiments, the cryopreservation medium contains a commercially available cryopreservation solution (CryoStor™ CS10). CryoStor™ CS10 is a cryopreservation medium containing 10% dimethyl sulfoxide (DMSO). In some embodiments, compositions formulated for cryopreservation can be stored at low temperatures, such as ultra-low temperatures, for example, storage with temperature ranges from −40° C. to −150° C., such as or about 80° C.±6.0° C.

It will be understood that following cryo preservation some cells in the population may be dead, such as up to 10%, up to 5%, up to 1%, up to 0.5% or up to 0.1% of cells. Alternatively, cell recovers after cryo preservation may be up to 20% or up to 10%.In some embodiments, the pharmaceutical composition comprises engineered cells described herein and a pharmaceutically acceptable carrier comprising 31.25% (v/v) Plasma-Lyte A, 31.25% (v/v) of 5% dextrose/0.45% sodium chloride, 10% dextran 40 (LMD)/5% dextrose, 20% (v/v) of 25% human serum albumin (HSA), and 7.5% (v/v) dimethylsulfoxide (DMSO).

In some embodiments, the cryopreserved engineered cells are prepared for administration by thawing. In some cases, the engineered cells can be administered to a subject immediately after thawing. In some such embodiments, the composition is ready-to-use without any further processing. In other cases, the engineered cells are further processed after thawing, such as by resuspension with a pharmaceutically acceptable carrier, incubation with an activating or stimulating agent, or are activated washed and resuspended in a pharmaceutically acceptable buffer prior to administration to a subject.

### III. Kits, Components, and Articles of Manufacture

In some aspects, provided herein are kits, components, and compositions (such as consumables) of the methods, devices, and systems described herein. In some embodiments, the kit comprises instructions for use according to the disclosure herein.

In some embodiments, provided herein is a kit comprising a population of engineered cells described herein. In some embodiments, provided herein is a kit comprising: (a) a population of cells comprising a plurality of engineered cells., and (ii) reduce expression of one or more MHC class I molecules and/or one or more MHC class II molecules (e.g., one or more MHC class I human leukocyte antigens and/or one or more MHC class II human leukocyte antigens), wherein the increased expression of (i) and the reduced expression of (ii) is relative to a cell of the same cell type that does not comprise the modifications. In some embodiments, provided herein is a kit or combination, comprising a population of cells comprising a plurality of engineered cells,; (ii) increase expression of CD47, and (iii) reduce expression of one or more MHC class I molecules and/or one or more MHC class II molecules (e.g., one or more MHC class I human leukocyte antigens and/or one or more MHC class II human leukocyte antigens), wherein the increased expression of (i) and (ii) and the reduced expression of (iii) is relative to a cell of the same cell type that does not comprise the modifications.

In some embodiments, there is provided an article of manufacture containing materials useful for clinical transplantation therapies, including cell therapies. In some embodiments, the articles of manufacture contain material useful for the treatment of cellular deficiencies, such as but not limited to diabetes (e.g., Type I diabetes), vascular conditions or disease, autoimmune thyroiditis, live disease (e.g., cirrhosis of the liver), corneal disease (e.g., Fuchs dystrophy or congenital hereditary endothelial dystrophy), kidney disease, and cancer (e.g., B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer). The article of manufacture can comprise a container and a label or package insert on or associated with the container. Suitable containers include, for example, bottles, vials, syringes, etc. (e.g., glass or plastic containers) Generally, the container holds a composition which is effective for allogenic cell therapy and may have a sterile access port (for example the container may be an intravenous solution bag or a vial having a stopper pierceable by a hypodermic injection needle).

In some aspects, a kit or article of manufacture provided herein comprises a population of engineered cells, such as any of the engineered cells provided herein. In some embodiments, a kit or article of manufacture comprises a composition comprising a population of engineered cells, wherein the engineered cells comprise: (i) a transgene comprising an exogenous polynucleotide encoding CD47, (ii) a, and (iii) inactivation or disruption of both alleles of a B2M gene. In some embodiments, the engineered beta cells further comprise inactivation or disruption of both alleles of a CIITA gene.

The label or package insert indicates that the composition is used for treating the particular condition. The label or package insert will further comprise instructions for administering the pharmaceutical composition to the patient. In some embodiments, the article of manufacture comprises a combination treatment.

The article of manufacture and/or kit may further comprise a package insert. The insert refers to instructions customarily included in commercial packages of therapeutic products that contain information about the indications, usage, dosage, administration, contraindications and/or warnings concerning the use of such therapeutic products.

### IV. Methods of Treatment

Provided herein are compositions and methods relating to the provided cell compositions comprising a population of engineered cells described herein for use in treating diseases or conditions in a subject. Provided herein is a method of treating a patient by administering a population engineered cells described herein. In some embodiments, the population of cells are formulated for administration in a pharmaceutical composition, such as any described here. Such methods and uses include therapeutic methods and uses, for example, involving administration of the population of engineered cells, or compositions containing the same, to a subject having a disease, condition, or disorder. It is within the level of a skilled artisan to choose the appropriate engineered cells as provided herein for a particular disease indication. In some embodiments, the cells or pharmaceutical composition thereof is administered in an effective amount to effect treatment of the disease or disorder. Uses include uses of the engineered cells or pharmaceutical compositions thereof in such methods and treatments, and in the preparation of a medicament in order to carry out such therapeutic methods. In some embodiments, the methods thereby treat the disease or condition or disorder in the subject.

The engineered cells provided herein can be administered to any suitable patients including, for example, a candidate for a cellular therapy for the treatment of a disease or disorder. Candidates for cellular therapy include any patient having a disease or condition that may potentially benefit from the therapeutic effects of the subject engineered cells provided herein. In some embodiments, the patient is an allogenic recipient of the administered cells. In some embodiments, the provided engineered cells are effective for use in allogeneic cell therapy. A candidate who benefits from the therapeutic effects of the subject engineered cells provided herein exhibit an elimination, reduction or amelioration of ta disease or condition.

In some embodiments, the engineered cells as provided herein, including those produced by any of the methods provided herein, can be used in cell therapy. Therapeutic cells outlined herein are useful to treat a disorder such as, but not limited to, a cancer, a genetic disorder, a chronic infectious disease, an autoimmune disorder, a neurological disorder, and the like.

In some embodiments, the patient has a cellular deficiency. As used herein, a “cellular deficiency” refers to any disease or condition that causes a dysfunction or loss of a population of cells in the patient, wherein the patient is unable to naturally replace or regenerate the population of cells. Exemplary cellular deficiencies include, but are not limited to, autoimmune diseases (e.g., multiple sclerosis, myasthenia gravis, rheumatoid arthritis, diabetes, systemic lupus and erythematosus), neurodegenerative diseases (e.g., Huntington's disease and Parkinson's disease), cardiovascular conditions and diseases, vascular conditions and diseases, corneal conditions and diseases, liver conditions and diseases, thyroid conditions and diseases, and kidney conditions and diseases. In some embodiments, the patient administered the engineered cells has a cancer. Exemplary cancers that can be treated by the engineered cells provided herein include, but are not limited to, B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer. In certain embodiments, the cancer patient is treated by administration of an engineered CAR T-cell provided herein.

In some embodiments, provided herein is a method of administering a population of engineered cells to a patient in need thereof, wherein the engineered cells come into contact with the blood during or after administration, and wherein the engineered cells comprise modifications that prevent or attenuate an IBMIR when the engineered cells come into contact with the blood. In some embodiments, the engineered cells are administered intravenously or via intramuscular injection. In some embodiments, the engineered cells overexpress a tolerogenic factor (e.g., CD47), have reduced expression or lack expression of one or more MHC class I molecules. In some embodiments, the engineered cells are beta islet cells or hepatocytes. In some embodiments, the engineered cells further comprise overexpression of one or more complement inhibitor.

In some embodiments, the cellular deficiency is associated with diabetes or the cellular therapy is for the treatment of diabetes, optionally wherein the diabetes is Type I diabetes. In some embodiments, the population of engineered cells is a population of islet cells, including beta islet cells. In some embodiments, the islet cells are selected from the group consisting of an islet progenitor cell, an immature islet cell, and a mature islet cell. In some embodiments, the method comprises administering to the patient a composition comprising a population of engineered beta islet cells, wherein the engineered cells comprise: (i) a multicistronic vector comprising an exogenous polynucleotide encoding CD47, and (ii) inactivation or disruption of both alleles of a B2M gene. In some embodiments, the method comprises administering to the patient a composition comprising a population of engineered beta islet cells, wherein the engineered beta islet cells comprise: (i) a transgene comprising an exogenous polynucleotide encoding CD47. In some embodiments, the engineered beta cells comprise inactivation or disruption of both alleles of a CIITA gene. In some embodiments, the transgene comprising the polynucleotide encoding CD47 is the transgene is a multicistronic vector.

In some embodiments, the cellular deficiency is associated with a liver disease or the cellular therapy is for the treatment of liver disease. In some embodiments, the liver disease comprises cirrhosis of the liver. In some embodiments, the population of cells is a population of hepatocytes or hepatic progenitor cells. In some embodiments, the method comprises administering to the patient a composition comprising a population of engineered hepatocyte cells, wherein the engineered hepatocyte cells comprise: (i) a transgene comprising an exogenous polynucleotide encoding CD47, and (iii) inactivation or disruption of both alleles of a B2M gene. In some embodiments, the method comprises administering to the patient a composition comprising a population of engineered hepatocyte cells, wherein the engineered hepatocyte cells comprise: (i) a transgene comprising an exogenous polynucleotide encoding CD47 and (ii) inactivation or disruption of both alleles of a B2M gene. In some embodiments, the engineered hepatocyte cells comprise inactivation or disruption of both alleles of a CIITA gene. In some embodiments, the transgene comprising the polynucleotide encoding CD47 is the transgene is a multicistronic vector.

In some embodiments, the engineered cells, or a composition containing the same, provided herein are useful for the treatment of a patient sensitized from one or more antigens present in a previous transplant such as, for example, a cell transplant, a blood transfusion, a tissue transplant, or an organ transplant. In certain embodiments, the previous transplant is an allogeneic transplant and the patient is sensitized against one or more alloantigens from the allogeneic transplant. Allogeneic transplants include, but are not limited to, allogeneic cell transplants, allogeneic blood transfusions, allogeneic tissue transplants, or allogeneic organ transplants. In some embodiments, the patient is sensitized patient who is or has been pregnant (e.g., having or having had alloimmunization in pregnancy). In certain embodiments, the patient is sensitized from one or more antigens included in a previous transplant, wherein the previous transplant is a modified human cell, tissue or organ. In some embodiments, the modified human cell, tissue or organ is a modified autologous human cell, tissue or organ. In some embodiments, the previous transplant is a non-human cell, tissue or organ. In exemplary embodiments, the previous transplant is a modified non-human cell, tissue, or organ. In certain embodiments, the previous transplant is a chimera that includes a human component. In certain embodiments, the previous transplant is a CAR T-cell. In certain embodiments, the previous transplant is an autologous transplant and the patient is sensitized against one or more autologous antigens from the autologous transplant. In certain embodiments, the previous transplant is an autologous cell, tissue or organ. In certain embodiments, the sensitized patient has an allergy and is sensitized to one or more allergens. In exemplary embodiments, the patient has a hay fever, a food allergy, an insect allergy, a drug allergy or atopic dermatitis.

In some embodiments, the patient undergoing a treatment using the provided engineered cells, or a composition containing the same, received a previous treatment. In some embodiments, the engineered cells, or a composition containing the same, are used to treat the same condition as the previous treatment. In certain embodiments, the engineered cells, or a composition containing the same, are used to treat a different condition from the previous treatment. In some embodiments, the engineered cells, or a composition containing the same, administered to the patient exhibit an enhanced therapeutic effect for the treatment of the same condition or disease treated by the previous treatment. In certain embodiments, the administered engineered cells, or a composition containing the same, exhibit a longer therapeutic effect for the treatment of the condition or disease in the patient as compared to the previous treatment. In exemplary embodiments, the administered cells exhibit an enhanced potency, efficacy and/or specificity against the cancer cells as compared to the previous treatment. In embodiments, the engineered cells are CAR T-cells for the treatment of a cancer.

The methods provided herein can be used as a second-line treatment for a particular condition or disease after a failed first line treatment. In some embodiments, the previous treatment is a therapeutically ineffective treatment. As used herein, a “therapeutically ineffective” treatment refers to a treatment that produces a less than desired clinical outcome in a patient. For example, with respect to a treatment for a cellular deficiency, a therapeutically ineffective treatment may refer to a treatment that does not achieve a desired level of functional cells and/or cellular activity to replace the deficient cells in a patient, and/or lacks therapeutic durability. With respect to a cancer treatment, a therapeutically ineffective treatment refers to a treatment that does not achieve a desired level of potency, efficacy and/or specificity. Therapeutic effectiveness can be measured using any suitable technique known in the art. In some embodiments, the patient produces an immune response to the previous treatment. In some embodiments, the previous treatment is a cell, tissue or organ graft that is rejected by the patient. In some embodiments, the previous treatment included a mechanically assisted treatment. In some embodiments, the mechanically assisted treatment included a hemodialysis or a ventricle assist device. In some embodiments, the patient produced an immune response to the mechanically assisted treatment. In some embodiments, the previous treatment included a population of therapeutic cells that include a safety switch that can cause the death of the therapeutic cells should they grow and divide in an undesired manner. In certain embodiments, the patient produces an immune response as a result of the safety switch induced death of therapeutic cells. In certain embodiments, the patient is sensitized from the previous treatment. In exemplary embodiments, the patient is not sensitized by the administered engineered cells as provided herein.

In some embodiments, the provided engineered cells, or compositions containing the same, are administered prior to providing a tissue, organ or partial organ transplant to a patient in need thereof. In embodiments, the patient does not exhibit an immune response to the engineered cells. In certain embodiments, the engineered cells are administered to the patient for the treatment of a cellular deficiency in a particular tissue or organ and the patient subsequently receives a tissue or organ transplant for the same particular tissue or organ. In such embodiments, the engineered cell treatment functions as a bridge therapy to the eventual tissue or organ replacement. For example, in some embodiments, the patient has a liver disorder and receives an engineered hepatocyte treatment as provided herein, prior to receiving a liver transplant. In certain embodiments, the engineered cells are administered to the patient for the treatment of a cellular deficiency in a particular tissue or organ and the patient subsequently receives a tissue or organ transplant for a different tissue or organ. For example, in some embodiments, the patient is a diabetes patient who is treated with engineered pancreatic beta cells as provided herein prior to receiving a kidney transplant. In some embodiments, the method is for the treatment of a cellular deficiency. In exemplary embodiments, the tissue or organ transplant is a heart transplant, a lung transplant, a kidney transplant, a liver transplant, a pancreas transplant, an intestine transplant, a stomach transplant, a cornea transplant, a bone marrow transplant, a blood vessel transplant, a heart valve transplant, or a bone transplant.

The methods of treating a patient are generally through administrations of engineered cells, or a composition containing the same, as provided herein. As will be appreciated, for all the multiple embodiments described herein related to the cells and/or the timing of therapies, the administering of the cells is accomplished by a method or route that results in at least partial localization of the introduced cells at a desired site. The cells can be implanted directly to the desired site, or alternatively be administered by any appropriate route which results in delivery to a desired location in the subject where at least a portion of the implanted cells or components of the cells remain viable. In some embodiments, the cells are administered to treat a disease or disorder, such as any disease, disorder, condition, or symptom thereof that can be alleviated by cell therapy.

In some embodiments, the population of engineered cells, or a composition containing the same, is administered at least 1 day, at least 2 days, at least 3 days, at least 4 days, at least 5, days, at least 6 days, at least 1 week, or at least 1 month or more after the patient is sensitized. In some embodiments, the population of engineered cells, or a composition containing the same, is administered at least 1 week (e.g., 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or more) or more after the patient is sensitized or exhibits characteristics or features of sensitization. In some embodiments, the population of engineered cells, or a composition containing the same, is administered at least 1 month (e.g., 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, or more) or more after the patient has received the transplant (e.g., an allogeneic transplant), has been pregnant (e.g., having or having had alloimmunization in pregnancy) or is sensitized or exhibits characteristics or features of sensitization.

In some embodiments, the patient who has received a transplant, who has been pregnant (e.g., having or having had alloimmunization in pregnancy), and/or who is sensitized against an antigen (e.g., alloantigens) is administered a dosing regimen comprising a first dose administration of a population of engineered cells described herein, a recovery period after the first dose, and a second dose administration of a population of engineered cells described. In some embodiments, the composite of cell types present in the first population of cells and the second population of cells are different. In certain embodiments, the composite of cell types present in the first population of engineered cells and the second population of engineered cells are the same or substantially equivalent. In many embodiments, the first population of engineered cells and the second population of engineered cells comprises the same cell types. In some embodiments, the first population of engineered cells and the second population of engineered cells comprises different cell types. In some embodiments, the first population of engineered cells and the second population of engineered cells comprises the same percentages of cell types. In other embodiments, the first population of engineered cells and the second population of cells comprises different percentages of cell types.

In some embodiments, the recovery period begins following the first administration of the population of engineered cells or a composition containing the same, and ends when such cells are no longer present or detectable in the patient. In some embodiments, the duration of the recovery period is at least 1 week (e.g., 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or more) or more after the initial administration of the cells. In some embodiments, the duration of the recovery period is at least 1 month (e.g., 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, or more) or more after the initial administration of the cells.

In some embodiments, the administered population of engineered cells, or a composition containing the same, is hypoimmunogenic when administered to the subject. In some embodiments, the engineered cells are hypoimmune. In some embodiments, an immune response against the engineered cells is reduced or lower by at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of the immune response produced by the administration of immunogenic cells (e.g., a population of cells of the same or similar cell type or phenotype but that do not contain the modifications, e.g., genetic modifications, of the engineered cells). In some embodiments, the administered population of engineered cells, or a composition containing the same, fails to elicit an immune response against the engineered cells in the patient.

In some embodiments, the administered population of engineered cells, or a composition containing the same, elicits a decreased or lower level of systemic TH1 activation in the patient. In some instances, the level of systemic TH1 activation elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of systemic TH1 activation produced by the administration of immunogenic cells (e.g., a population of cells of the same or similar cell type or phenotype but that do not contain the modifications, e.g., genetic modifications, of the engineered cells). In some embodiments, the administered population of engineered cells, or a composition containing the same, fails to elicit systemic TH1 activation in the patient.

In some embodiments, the administered population of engineered cells, or a composition containing the same, elicits a decreased or lower level of immune activation of peripheral blood mononuclear cells (PBMCs) in the patient. In some instances, the level of immune activation of PBMCs elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of immune activation of PBMCs produced by the administration of immunogenic cells (e.g., a population of cells of the same or similar cell type or phenotype but that do not contain the modifications, e.g., genetic modifications, of the engineered cells). In some embodiments, the administered population of engineered cells, or a composition containing the same, fails to elicit immune activation of PBMCs in the patient.

In some embodiments, the administered population of engineered cells, or a composition containing the same, elicits a decreased or lower level of donor-specific IgG antibodies in the patient. In some instances, the level of donor-specific IgG antibodies elicited by the cells is at least 5%, 10%, 15%, 20%, 25%, 30%, 35%,40%, 45%, 50%, 55%, 60%, 65%, 70%, 75%, 80%, 85%, 90%, 91%, 92%, 93%, 94%, 95%, 96%, 97%, 98%, or 99% lower compared to the level of donor-specific IgG antibodies produced by the administration of immunogenic cells (e.g., a population of cells of the same or similar cell type or phenotype but that do not contain the modifications, e.g., genetic modifications, of the engineered cells). In some embodiments, the administered population of engineered cells fails to elicit donor-specific IgG antibodies in the patient.

In some embodiments, the administered population of engineered cells, or a composition containing the same, elicits a decreased or lower level of IgM and IgG antibody production in the patient. In some instances, the level of IgM and IgG antibody production elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of IgM and IgG antibody production produced by the administration of immunogenic cells (e.g., a population of cells of the same or similar cell type or phenotype but that do not contain the modifications, e.g., genetic modifications, of the engineered cells). In some embodiments, the administered population of engineered cells, or a composition containing the same, fails to elicit IgM and IgG antibody production in the patient.

In some embodiments, the administered population of engineered cells, or a composition containing the same, elicits a decreased or lower level of cytotoxic T cell killing in the patient. In some instances, the level of cytotoxic T cell killing elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of cytotoxic T cell killing produced by the administration of immunogenic cells (e.g., a population of cells of the same or similar cell type or phenotype but that do not contain the modifications, e.g., genetic modifications, of the engineered cells). In some embodiments, the administered population of engineered cells, or a composition containing the same, fails to elicit cytotoxic T cell killing in the patient.

As discussed above, provided herein are cells that in certain embodiments can be administered to a patient sensitized against alloantigens such as human leukocyte antigens. In some embodiments, the patient is or has been pregnant, e.g., with alloimmunization in pregnancy (e.g., hemolytic disease of the fetus and newborn (HDFN), neonatal alloimmune neutropenia (NAN) or fetal and neonatal alloimmune thrombocytopenia (FNAIT)). In other words, the patient has or has had a disorder or condition associated with alloimmunization in pregnancy such as, but not limited to, hemolytic disease of the fetus and newbom (HDFN), neonatal alloimmune neutropenia (NAN), and fetal and neonatal alloimmune thrombocytopenia (FNAIT). In some embodiments, the patient has received an allogeneic transplant such as, but not limited to, an allogeneic cell transplant, an allogeneic blood transfusion, an allogeneic tissue transplant, or an allogeneic organ transplant. In some embodiments, the patient exhibits memory B cells against alloantigens. In some embodiments, the patient exhibits memory T cells against alloantigens. Such patients can exhibit both memory B and memory T cells against alloantigens.

Upon administration of the cells described, the patient exhibits no systemic immune response or a reduced level of systemic immune response compared to responses to cells that are not hypoimmunogenic. In some embodiments, the patient exhibits no adaptive immune response or a reduced level of adaptive immune response compared to responses to cells that are not hypoimmunogenic. In some embodiments, the patient exhibits no innate immune response or a reduced level of innate immune response compared to responses to cells that are not hypoimmunogenic. In some embodiments, the patient exhibits no T cell response or a reduced level of T cell response compared to responses to cells that are not hypoimmunogenic. In some embodiments, the patient exhibits no B cell response or a reduced level of B cell response compared to responses to cells that are not hypoimmunogenic.

### A. Dose and Dosage Regimen

Any therapeutically effective amount of cells described herein can be included in the pharmaceutical composition, depending on the indication being treated. Non-limiting examples of the cells include primary cells (e.g., primary beta islet cells) and cells differentiated from engineered induced pluripotent stem cells as described (e.g., beta islet cells or hepatocytes differentiated from iPSCs). In some embodiments, the pharmaceutical composition includes at least about 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, or 5×10¹⁰ cells. In some embodiments, the pharmaceutical composition includes up to about 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, or 5×10¹⁰ cells. In some embodiments, the pharmaceutical composition includes up to about 6.0×10⁸ cells. In some embodiments, the pharmaceutical composition includes up to about 8.0×10⁸ cells. In some embodiments, the pharmaceutical composition includes at least about 1×10²-5×10², 5×10²-1×10³, 1×10³-5×10³, 5×10³-1×10⁴, 1×10⁴-5×10⁴, 5×10⁴-1×10⁵, 1×10⁵-5×10⁵, 5×10⁵-1×10⁶, 1×10⁶-5×10⁶, 5×10⁶-1×10⁷, 1×10⁷-5×10⁷, 5×10⁷-1×10⁸, 1×10⁸-5×10⁸, 5×10⁸-1×10⁹, 1×10⁹-5×10⁹, 5×10⁹-1×10¹⁰, or 1×10¹⁰-5×10¹⁰ cells. In exemplary embodiments, the pharmaceutical composition includes from about 1.0×10⁶ to about 2.5×10⁸ cells.

In some embodiments, the pharmaceutical composition has a volume of at least 5, 10, 15, 20, 25, 30, 35, 40, 45, 50, 55, 60, 65, 70, 75, 80. 85, 90, 95, 100, 110, 120, 130, 140, 150, 160, 170, 180, 190, 200, 250, 300, 350, 400, or 500 ml. In exemplary embodiments, the pharmaceutical composition has a volume of up to about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, or 500 ml. In exemplary embodiments, the pharmaceutical composition has a volume of about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, or 500 ml. In some embodiments, the pharmaceutical composition has a volume of from about 1-50 ml, 50-100 ml, 100-150 ml, 150-200 ml, 200-250 ml, 250-300 ml, 300-350 ml, 350-400 ml, 400-450 ml, or 450-500 ml. In some embodiments, the pharmaceutical composition has a volume of from about 1-50 ml, 50-100 ml, 100-150 ml, 150-200 ml, 200-250 ml, 250-300 ml, 300-350 ml, 350-400 ml, 400-450 ml, or 450-500 ml. In some embodiments, the pharmaceutical composition has a volume of from about 1-10 ml, 10-20 ml, 20-30 ml, 30-40 ml, 40-50 ml, 50-60 ml, 60-70 ml, 70-80 ml, 70-80 ml, 80-90 ml, or 90-100 ml. In some embodiments, the pharmaceutical composition has a volume that ranges from about 5 ml to about 80 ml. In exemplary embodiments, the pharmaceutical composition has a volume that ranges from about 10 ml to about 70 ml. In many embodiments, the pharmaceutical composition has a volume that ranges from about 10 ml to about 50 ml.

The specific amount/dosage regimen will vary depending on the weight, gender, age and health of the individual; the formulation, the biochemical nature, bioactivity, bioavailability and the side effects of the cells and the number and identity of the cells in the complete therapeutic regimen.

In some embodiments, a dose of the pharmaceutical composition includes about 1.0×10⁵ to about 2.5×10⁸ cells at a volume of about 10 mL to 50 mL and the pharmaceutical composition is administered as a single dose.

In many embodiments, the cells are T cells and the pharmaceutical composition includes from about 2.0×10⁶ to about 2.0×10⁸ cells, such as but not limited to, primary T cells, T cells differentiated from engineered induced pluripotent stem cells. In some cases, the dose includes about 1.0×10⁵ to about 2.5×10⁸ primary T cells described herein at a volume of about 10 ml to 50 ml. In several cases, the dose includes about 1.0×10⁵ to about 2.5×10⁸ primary T cells that have been described above at a volume of about 10 ml to 50 ml. In various cases, the dose includes about 1.0×10⁵ to about 2.5×10⁸ T cells differentiated from engineered induced pluripotent stem cells described herein at a volume of about 10 ml to 50 ml. In other cases, the dose is at a range that is lower than about 1.0×10⁵ to about 2.5×10⁸ T cells, including primary T cells or T cells differentiated from engineered induced pluripotent stem cells. In yet other cases, the dose is at a range that is higher than about 1.0×10⁵ to about 2.5 ×10⁸ T cells, including primary T cells and T cells differentiated from engineered induced pluripotent stem cells.

In some embodiments, the pharmaceutical composition is administered as a single dose of from about 1.0×10⁵ to about 1.0×10⁷ engineered cells (such as primary cells or cells differentiated from engineered induced pluripotent stem cells) per kg body weight for subjects 50 kg or less. In some embodiments, the pharmaceutical composition is administered as a single dose of from about 0.5×10⁵ to about 1.0×10⁷, about 1.0×10⁵ to about 1.0×10⁷, about 1.0 ×10⁵ to about 1.0×10⁷, about 5.0×10⁵ to about 1×10⁷, about 1.0×10⁶ to about 1×10⁷, about 5.0×10⁶ to about 1.0×10⁷, about 1.0×10⁵ to about 5.0×10⁶, about 1.0×10⁵ to about 1.0×10⁶, about 1.0×10⁵ to about 5.0×10⁵, about 1.0×10⁵ to about 5.0×10⁶, about 2.0×10⁵ to about 5.0×10⁶, about 3.0 ×10⁵ to about 5.0×10⁶, about 4.0×10⁵ to about 5.0×10⁶, about 5.0×10⁵ to about 5.0×10⁶, about 6.0×10⁵ to about 5.0×10⁶, about 7.0×10⁵ to about 5.0×10⁶, about 8.0×10⁵ to about 5.0×10⁶, or about 9.0×10⁵ to about 5.0×10⁶ cells per kg body weight for subjects 50 kg or less. In some embodiments, the dose is from about 0.2×10⁶ to about 5.0×10⁶ cells per kg body weight for subjects 50 kg or less. In many embodiments, the dose is at a range that is lower than from about 0.2×10⁶ to about 5.0×10⁶ cells per kg body weight for subjects 50 kg or less. In many embodiments, the dose is at a range that is higher than from about 0.2×10⁶ to about 5.0×10⁶ cells per kg body weight for subjects 50 kg or less. In exemplary embodiments, the single dose is at a volume of about 10 ml to 50 ml. In some embodiments, the dose is administered intravenously.

In exemplary embodiments, the cells are administered in a single dose of from about 1.0×10⁶ to about 5.0×10⁸ cells (such as primary cells and cells differentiated from engineered induced pluripotent stem cells) for subjects above 50 kg. In some embodiments, the pharmaceutical composition is administered as a single dose of from about 0.5×10⁶ to about 1.0×10⁹, about 1.0×10⁶ to about 1.0×10⁹, about 1.0×10⁶ to about 1.0×10⁹, about 5.0×10⁶ to about 1.0×10⁹, about 1.0×10⁷ to about 1.0×10⁹, about 5.0×10⁷ to about 1.0×10⁹, about 1.0×10⁶ to about 5.0×10⁷, about 1.0×10⁶ to about 1.0×10⁷, about 1.0×10⁶ to about 5.0×10⁷, about 1.0×10⁷ to about 5.0×10⁸, about 2.0×10⁷ to about 5.0×10⁸, about 3.0×10⁷ to about 5.0×10⁸, about 4.0×10⁷ to about 5.0×10⁸, about 5.0×10⁷ to about 5.0×10⁸, about 6.0×10⁷ to about 5.0×10⁸, about 7.0×10⁷ to about 5.0×10⁸, about 8.0×10⁷ to about 5.0×10⁸, or about 9.0×10⁷ to about 5.0×10⁸ cells per kg body weight for subjects 50 kg or less. In many embodiments, the cells are administered in a single dose of about 1.0×10⁷ to about 2.5 ×10⁸ cells for subjects above 50 kg. In some embodiments, the cells are administered in a single dose of a range that is less than about 1.0×10⁷ to about 2.5×10⁸cells for subjects above 50 kg. In some embodiments, the cells are administered in a single dose of a range that is higher than about 1.0×10⁷ to about 2.5×10⁸ cells for subjects above 50 kg. In some embodiments, the dose is administered intravenously. In exemplary embodiments, the single dose is at a volume of about 10 ml to 50 ml. In some embodiments, the dose is administered intravenously.

In exemplary embodiments, the dose is administered intravenously at a rate of about 1 to 50 ml per minute, 1 to 40 ml per minute, 1 to 30 ml per minute, 1 to 20 ml per minute, 10 to 20 ml per minute, 10 to 30 ml per minute, 10 to 40 ml per minute, 10 to 50 ml per minute, 20 to 50 ml per minute, 30 to 50 ml per minute, 40 to 50 ml per minute. In numerous embodiments, the pharmaceutical composition is stored in one or more infusion bags for intravenous administration. In some embodiments, the dose is administered completely at no more than 10 minutes, 15 minutes, 20 minutes, 25 minutes, 30 minutes, 35 minutes, 40 minutes, 45 minutes, 50 minutes, 55 minutes, 60 minutes, 70 minutes, 80 minutes, 90 minutes, 120 minutes, 150 minutes, 180 minutes, 240 minutes, or 300 minutes.

In some embodiments, a single dose of the pharmaceutical composition is present in a single infusion bag. In other embodiments, a single dose of the pharmaceutical composition is divided into 2, 3, 4 or 5 separate infusion bags.

In some embodiments, the cells described herein are administered in a plurality of doses such as 2, 3, 4, 5, 6 or more doses. In some embodiments, each dose of the plurality of doses is administered to the subject ranging from 1 to 24 hours apart. In some instances, a subsequent dose is administered from about 1 hour to about 24 hours (e.g., about 1, 2, 3, 4, 5, 6, 7, 8, 9, 10, 11, 12, 13, 14, 15, 16, 17, 18, 19, 20, 21, 22, 23 or about 24 hours) after an initial or preceding dose. In some embodiments, each dose of the plurality of doses is administered to the subject ranging from about 1 day to 28 days apart. In some instances, a subsequent dose is administered from about 1 day to about 28 days (e.g., about 1, 2, 3, 4, 5, 6, 7, 8, 9, 10, 11, 12, 13, 14, 15, 16, 17, 18, 19, 20, 21, 22, 23, 24, 25, 26, 27, or about 28 days) after an initial or preceding dose. In many embodiments, each dose of the plurality of doses is administered to the subject ranging from 1 week to about 6 weeks apart. In certain instances, a subsequent dose is administered from about 1 week to about 6 weeks (e.g., about 1, 2, 3, 4, 5, or 6 weeks) after an initial or preceding dose. In several embodiments, each dose of the plurality of doses is administered to the subject ranging from about 1 month to about 12 months apart. In several instances, a subsequent dose is administered from about 1 month to about 12 months (e.g., about 1, 2, 3, 4, 5, 6, 7, 8, 9, 10, 11, or 12 months) after an initial or preceding dose.

In some embodiments, a subject is administered a first dosage regimen at a first timepoint, and then subsequently administered a second dosage regimen at a second timepoint. In some embodiments, the first dosage regimen is the same as the second dosage regimen. In other embodiments, the first dosage regimen is different than the second dosage regimen. In some instances, the number of cells in the first dosage regimen and the second dosage regimen are the same. In some instances, the number of cells in the first dosage regimen and the second dosage regimen are different. In some cases, the number of doses of the first dosage regimen and the second dosage regimen are the same. In some cases, the number of doses of the first dosage regimen and the second dosage regimen are different.

In some embodiments, the cells are engineered T cells (e.g., primary T cells or T cells differentiated from engineered induced pluripotent stem cells) and the first dosage regimen includes engineered T cells expressing a first CAR and the second dosage regimen includes engineered T cells expressing a second CAR such that the first CAR and the second CAR are different. For instance, the first CAR and second CAR bind different target antigens. In some cases, the first CAR includes an scFv that binds an antigen and the second CAR includes an scFv that binds a different antigen. In some embodiments, the first dosage regimen includes engineered T cells expressing a first CAR and the second dosage regimen includes engineered T cells or primary T cells expressing a second CAR such that the first CAR and the second CAR are the same. The first dosage regimen can be administered to the subject at least 1 month, 2 months, 3 months, 4 months, 5 months, 6 months, 7 months, 8 months, 9 months, 10 months, 11 months, 12 months, 1-3 months, 1-6 months, 4-6 months, 3-9 months, 3-12 months, or more months apart from the second dosage regimen. In some embodiments, a subject is administered a plurality of dosage regimens during the course of a disease (e.g., cancer) and at least two of the dosage regimens comprise the same type of engineered T cells described herein. In other embodiments, at least two of the plurality of dosage regimens comprise different types of engineered T cells described herein.

### B. Immunosuppressive Agent

In some embodiments, an immunosuppressive and/or immunomodulatory agent is not administered to the patient before the first administration of the population of engineered cells, or in a composition containing the same.

In some embodiments, an immunosuppressive and/or immunomodulatory agent may be administered to a patient received administration of engineered cells. In some embodiments, the immunosuppressive and/or immunomodulatory agent is administered prior to administration of the engineered cells. In some embodiments, the immunosuppressive and/or immunomodulatory agent is administered prior to administration of a first and/or second administration of engineered cells.

Non-limiting examples of an immunosuppressive and/or immunomodulatory agent include cyclosporine, azathioprine, mycophenolic acid, mycophenolate mofetil, corticosteroids such as prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin, thymosin-α and similar agents. In some embodiments, the immunosuppressive and/or immunomodulatory agent is selected from a group of immunosuppressive antibodies consisting of antibodies binding to p75 of the IL-2 receptor, antibodies binding to, for instance, MHC, CD2, CD3, CD4, CD7, CD28, B7, CD40, CD45, IFN-gamma, TNF-.alpha., IL-4, IL-5, IL-6R, IL-6, IGF, IGFR1, IL-7, IL-8, IL-10, CD11a, or CD58, and antibodies binding to any of their ligands. In some embodiments where an immunosuppressive and/or immunomodulatory agent is administered to the patient before or after the first administration of the cells, the administration is at a lower dosage than would be required for cells with MHC class I and/or MHC class II expression and without exogenous expression of CD47.

In some embodiments, such an immunosuppressive and/or immunomodulatory agent may be selected from soluble IL-15R, IL-10. B7 molecules (e.g., B7-1. B7-2, variants thereof, and fragments thereof), ICOS, and OX40, an inhibitor of a negative T cell regulator (such as an antibody against CTLA-4) and similar agents.

In some embodiments, an immunosuppressive and/or immunomodulatory agent can be administered to the patient before the first administration of the population of engineered cells. In some embodiments, an immunosuppressive and/or immunomodulatory agent is administered at least 1, 2, 3, 4, 5, 6, 7, 8, 9, 10, 11, 12, 13, 14 days or more before the first administration of the cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more before the first administration of the cells.

In embodiments, an immunosuppressive and/or immunomodulatory agent is not administered to the patient after the first administration of the cells, or is administered at least 1, 2, 3, 4, 5, 6, 7, 8, 9, 10, 11, 12, 13, 14 days or more after the first administration of the cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more after the first administration of the cells.

In some embodiments, an immunosuppressive and/or immunomodulatory agent is not administered to the patient before the administration of the population of engineered cells. In many embodiments, an immunosuppressive and/or immunomodulatory agent is administered to the patient before the first and/or second administration of the population of engineered cells. In some embodiments, an immunosuppressive and/or immunomodulatory agent is administered at least 1, 2, 3, 4, 5, 6, 7, 8, 9, 10, 11, 12, 13, 14 days or more before the administration of the cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more before the first and/or second administration of the cells. In embodiments, an immunosuppressive and/or immunomodulatory agent is administered at least 1, 2, 3, 4, 5, 6, 7, 8, 9, 10, 11, 12, 13, 14 days or more after the administration of the cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more after the first and/or second administration of the cells.

In some embodiments where an immunosuppressive and/or immunomodulatory agent is administered to the patient before or after the administration of the cells, the administration is at a lower dosage than would be required for immunogenic cells (e.g., a population of cells of the same or similar cell type or phenotype but that do not contain the modifications, e.g., genetic modifications, of the engineered cells, e.g., with endogenous levels of, MHC class I, and/or MHC class II expression and without increased (e.g., exogenous) expression of CD47).

### V. Computer Implemented Methods

Computing-based devices comprise one or more processors which are microprocessors, controllers or any other suitable type of processors for processing computer executable instructions to control the operation of the device in order to perform the claimed and disclosed methods. In some instances, for example where a system on a chip architecture is used, the processors include one or more fixed function blocks (also referred to as accelerators) which implement a part of the claimed and disclosed methods in hardware (rather than software or firmware). Platform software comprising an operating system or any other suitable platform software is provided at the computing-based device to enable application software to be executed on the device.

The computer executable instructions are provided using any computer-readable media that is accessible by computing based device. Computer-readable media includes, for example, computer storage media such as memory and communications media. Computer storage media, such as memory, includes volatile and non-volatile, removable and non-removable media implemented in any method or technology for storage of information such as computer readable instructions, data structures, program modules or the like. Computer storage media includes, but is not limited to, random access memory (RAM), read only memory (ROM), erasable programmable read only memory (EPROM), electronic erasable programmable read only memory (EEPROM), flash memory or other memory technology, compact disc read only memory (CD-ROM), digital versatile disks (DVD) or other optical storage, magnetic cassettes, magnetic tape, magnetic disk storage or other magnetic storage devices, or any other non-transmission medium that is used to store information for access by a computing device. In contrast, communication media embody computer readable instructions, data structures, program modules, or the like in a modulated data signal, such as a carrier wave, or other transport mechanism. As defined herein, computer storage media does not include communication media. Therefore, a computer storage medium should not be interpreted to be a propagating signal per se. Although the computer storage media (memory is shown within the computing-based device it will be appreciated that the storage is, in some examples, distributed or located remotely and accessed via a network or other communication link (e.g., using communication interface).

The computing-based device also comprises an input/output controller arranged to output display information to a display device which may be separate from or integral to the computing-based device. The display information may provide a graphical user interface. The input/output controller is also arranged to receive and process input from one or more devices, such as a user input device (e.g., a mouse, keyboard, camera, microphone or other sensor). In some examples the user input device detects voice input, user gestures or other user actions and provides a natural user interface (NUI). The display device may also act as the user input device if it is a touch sensitive display device. The input/output controller outputs data to devices other than the display device in some examples, e.g., a locally connected printing device.

Any of the input/output controller, display device and the user input device may comprise NUI technology which enables a user to interact with the computing-based device in a natural manner, free from artificial constraints imposed by input devices such as mice, keyboards, remote controls and the like. Examples ofNUI technology that are provided in some examples include but are not limited to those relying on voice and/or speech recognition, touch and/or stylus recognition (touch sensitive displays), gesture recognition both on screen and adjacent to the screen, air gestures, head and eye tracking, voice and speech, vision, touch, gestures, and machine intelligence. Other examples of NUI technology that are used in some examples include intention and goal understanding systems, motion gesture detection systems using depth cameras (such as stereoscopic camera systems, infrared camera systems, red green blue (rgb) camera systems and combinations of these), motion gesture detection using accelerometers/gyroscopes, facial recognition, three dimensional (3D) displays, head, eye and gaze tracking, immersive augmented reality and virtual reality systems and technologies for sensing brain activity using electric field sensing electrodes (electro encephalogram (EEG) and related methods).

The term ‘computer’ or ‘computing-based device’ is used herein to refer to any device with processing capability such that it executes instructions. Those skilled in the art will realize that such processing capabilities are incorporated into many different devices and therefore the terms ‘computer’ and ‘computing-based device’ each include personal computers (PCs), servers, mobile telephones (including smart phones), tablet computers, set-top boxes, media players, games consoles, personal digital assistants, wearable computers, and many other devices.

The methods described herein are performed, in some examples, by software in machine readable form on a tangible storage medium e.g., in the form of a computer program comprising computer program code means adapted to perform all the operations of one or more of the methods described herein when the program is run on a computer and where the computer program may be embodied on a computer readable medium. The software is suitable for execution on a parallel processor or a serial processor such that the method operations may be carried out in any suitable order, or simultaneously.

Those skilled in the art will realize that storage devices utilized to store program instructions are optionally distributed across a network. For example, a remote computer is able to store an example of the process described as software. A local or terminal computer is able to access the remote computer and download a part or all of the software to run the program. Alternatively, the local computer may download pieces of the software as needed, or execute some software instructions at the local terminal and some at the remote computer (or computer network). Those skilled in the art will also realize that by utilizing conventional techniques known to those skilled in the art that all, or a portion of the software instructions may be carried out by a dedicated circuit, such as a digital signal processor (DSP), programmable logic array, or the like.

In various examples the cell function model is a machine learning model such as a neural network, random decision forest, support vector machine or other machine learning model. In an example the machine learning model is a classifier which has been trained using supervised learning or semi-supervised learning and using a training data set. The training data set comprises a plurality of training examples where each training example is test data of a sample and a ground truth value of the class of the sample. The training examples are obtained empirically and/or are synthetic training examples computed using a software simulation. In the case of empirical training examples, these are obtained by taking assay readouts from samples obtained from human subjects. A range of different samples are obtained such as from human subjects from different demographic groups so as to avoid any unintentional bias in performance of the resulting machine learning model. For each sample used to form a training data example, a class of the sample (such as good donor capability or not good donor capability) is known from medical records of the human subject. The machine learning model is then trained using any suitable training algorithm and an objective function which takes into account differences between predictions of the machine learning model and the ground truth classes of the training examples. In an example where the machine learning model is a neural network such as a multi-layer perceptron or other type of neural network, the neural network is trained using backpropagation. The machine learning model is trained using the available training data items or until little change in parameters of the machine learning model are observed. Once the machine learning model has been trained it is deployed at any suitable computing device such as a hospital desktop computer or a web server in the cloud. The deployed machine learning model receives a test data item from a sample not previously used to train the machine learning model. The machine learning model processes the test data item to compute a prediction indicating which class of the plurality of classes the test data item falls into. In some cases the machine learning model also provides a confidence value indicating uncertainty associated with the prediction.

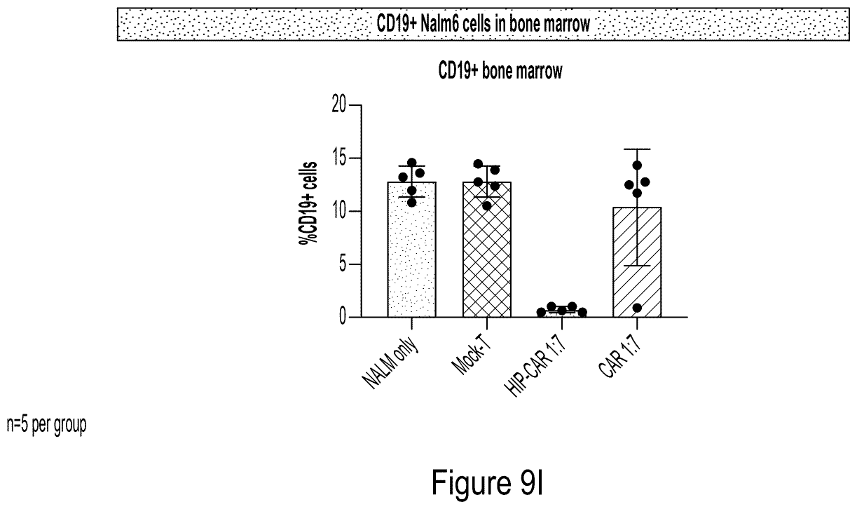

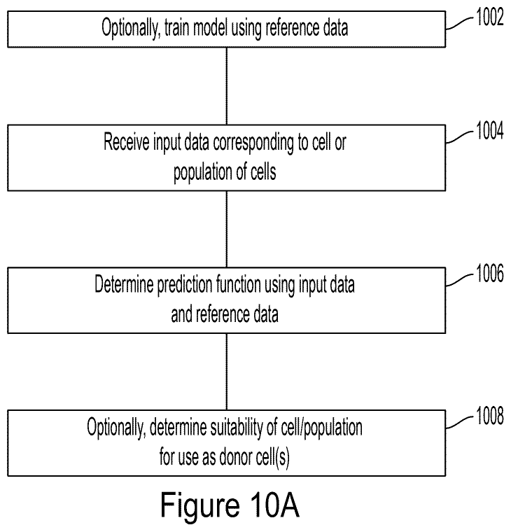

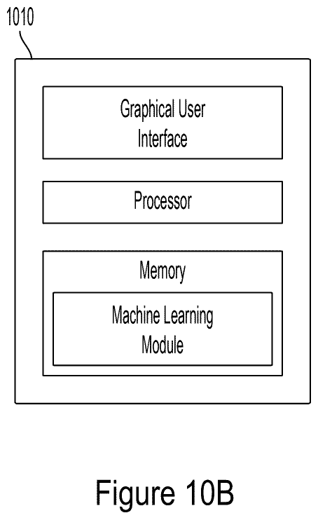

FIG. 10A is a flow chart of a method of evaluating a cell or a population of cells for a predicted function. In optional step 1002, a model that will be used to make predictions (e.g., in a machine learning module) is trained, for example using reference data corresponding to reference cells and/or populations of reference cells, for example generated using a method described elsewhere herein. In step 1004, input data corresponding to a cell or a population of cells (for which a prediction of function is desired) is received by a processor of a computing device. The input data may have been generated using a method described elsewhere herein. In step 1006, a predicted function of the cell or the population of cells is determined using the input data and reference data corresponding to reference cells and/or populations of reference cells. For example, the input data can be input into a machine learning module that has been trained using the reference data in order to make an inference of the predicted function. In optional step 1008, suitability of the cell or the population of cells for use as donor cell(s), for example for a cell therapy, is determined. In some embodiments, a machine learning module simultaneously predicts function and suitability or predicts function and suitability as part of a single process. For example, suitability may be (e.g., automatically) determined based on predicted function, for example using any of the aforementioned criteria. FIG. 10B illustrates an example system 1010 that can perform these and other methods. The system 1010 includes one or more processors, a memory storing one or more programs that include instructions that are executable by the processor(s), a machine learning module stored in the memory, and a graphical user interface. The graphical user interface may be used to receive user input (e.g., selection(s)) that is used in making determinations. For example, a user may select cell characteristic(s), such as function(s) and/or cell parameter(s), to weight.

A machine learning module may employ, for example, a regression-based model (e.g., a logistic regression model), a regularization-based model (e.g., an elastic net model or a ridge regression model), an instance-based model (e.g., a support vector machine or a k-nearest neighbor model), a Bayesian-based model (e.g., a naive-based model or a Gaussian naive-based model), a clustering-based model (e.g., an expectation maximization model), an ensemble-based model (e.g., an adaptive boosting model, a random forest model, a bootstrap-aggregation model, or a gradient boosting machine model), or a neural-network-based model (e.g., a convolutional neural network, a recurrent neural network, autoencoder, a back propagation network, or a stochastic gradient descent network). In embodiments, a machine learning model is trained using supervised learning algorithms, unsupervised learning algorithms, semi-supervised learning algorithms (e.g., partial supervision), weak supervision, transfer, multi-task learning, or any combination thereof. In embodiments, a machine learning module employs a model that comprises parameters (e.g., weights) that are tuned during training of the model. For example, the parameters may be adjusted to minimize a loss function, thereby improving the predictive capacity of the machine learning model.

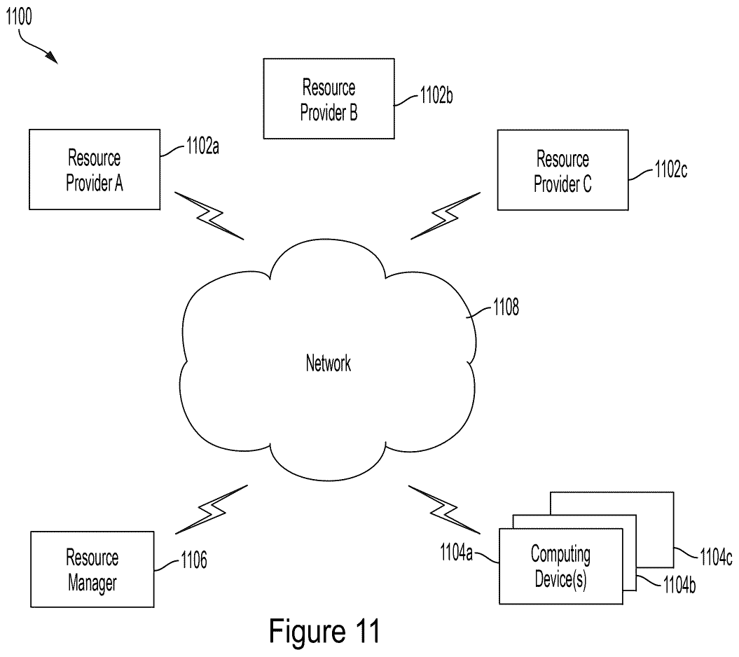

Illustrative embodiments of systems and methods disclosed herein were described above with reference to computations performed locally by a computing device (e.g., with a machine learning module). However, computations performed over a network are also contemplated. FIG. 11 shows an illustrative network environment 1100 for use in the methods and systems described herein. In brief overview, referring now to FIG. 11, a block diagram of an illustrative cloud computing environment 1100 is shown and described. The cloud computing environment 1100 may include one or more resource providers 1102a, 1102b, 1102c (collectively, 1102). Each resource provider 1102 may include computing resources. In some implementations, computing resources may include any hardware and/or software used to process data. For example, computing resources may include hardware and/or software capable of executing algorithms, computer programs, and/or computer applications. In some implementations, illustrative computing resources may include application servers and/or databases with storage and retrieval capabilities. Each resource provider 1102 may be connected to any other resource provider 1102 in the cloud computing environment 1100. In some implementations, the resource providers 1102 may be connected over a computer network 1108. Each resource provider 1102 may be connected to one or more computing device 1104a, 1104b, 1104c (collectively, 1104), over the computer network 1108.

The cloud computing environment 1100 may include a resource manager 1106. The resource manager 1106 may be connected to the resource providers 1102 and the computing devices 1104 over the computer network 1108. In some implementations, the resource manager 1106 may facilitate the provision of computing resources by one or more resource providers 1102 to one or more computing devices 1104. The resource manager 1106 may receive a request for a computing resource from a particular computing device 1104. The resource manager 1106 may identify one or more resource providers 1102 capable of providing the computing resource requested by the computing device 1104. The resource manager 1106 may select a resource provider 1102 to provide the computing resource. The resource manager 1106 may facilitate a connection between the resource provider 1102 and a particular computing device 1104. In some implementations, the resource manager 1106 may establish a connection between a particular resource provider 1102 and a particular computing device 1104. In some implementations, the resource manager 1106 may redirect a particular computing device 1104 to a particular resource provider 1102 with the requested computing resource.

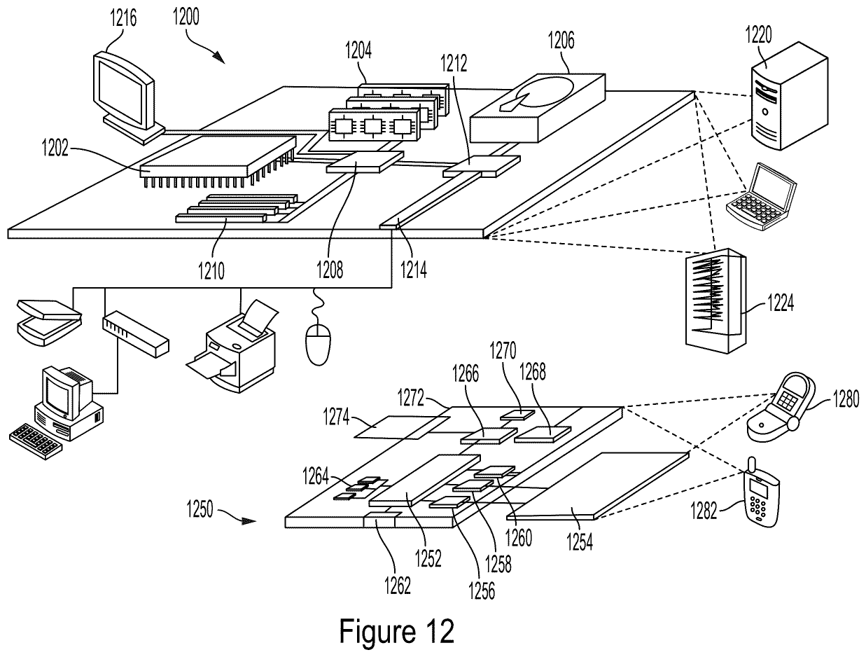

FIG. 12 shows an example of a computing device 1200 and a mobile computing device 1250 that can be used in the methods and systems described in this disclosure. The computing device 1200 is intended to represent various forms of digital computers, such as laptops, desktops, workstations, personal digital assistants, servers, blade servers, mainframes, and other appropriate computers. The mobile computing device 1250 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 1200 includes a processor 1202, a memory 1204, a storage device 1206, a high-speed interface 1208 connecting to the memory 1204 and multiple high-speed expansion ports 1210, and a low-speed interface 1212 connecting to a low-speed expansion port 1214 and the storage device 1206. Each of the processor 1202, the memory 1204, the storage device 1206, the high-speed interface 1208, the high-speed expansion ports 1210, and the low-speed interface 1212, are interconnected using various busses, and may be mounted on a common motherboard or in other manners as appropriate. The processor 1202 can process instructions for execution within the computing device 1200, including instructions stored in the memory 1204 or on the storage device 1206 to display graphical information for a GUI on an external input/output device, such as a display 1216 coupled to the high-speed interface 1208. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system). Also, multiple computing devices may be connected, with each device providing portions of the necessary operations (e.g., as a server bank, a group of blade servers, or a multi-processor system). Thus, as the term is used herein, where a plurality of functions are described as being performed by “a processor”, this encompasses embodiments wherein the plurality of functions are performed by any number of processors (e.g., one or more processors) of any number of computing devices (e.g., one or more computing devices). Furthermore, where a function is described as being performed by “a processor”, this encompasses embodiments wherein the function is performed by any number of processors (e.g., one or more processors) of any number of computing devices (e.g., one or more computing devices) (e.g., in a distributed computing system).

The memory 1204 stores information within the computing device 1200. In some implementations, the memory 1204 is a volatile memory unit or units. In some implementations, the memory 1204 is a non-volatile memory unit or units. The memory 1204 may also be another form of computer-readable medium, such as a magnetic or optical disk.

The storage device 1206 is capable of providing mass storage for the computing device 1200. In some implementations, the storage device 1206 may be or contain a computer-readable medium, such as a hard disk device, an optical disk device, a flash memory or other similar solid state memory device, or an array of devices, including devices in a storage area network or other configurations. Instructions can be stored in an information carrier. The instructions, when executed by one or more processing devices (for example, processor 1202), perform one or more methods, such as those described above. The instructions can also be stored by one or more storage devices such as computer- or machine-readable mediums (for example, the memory 1204, the storage device 1206, or memory on the processor 1202).

The high-speed interface 1208 manages bandwidth-intensive operations for the computing device 1200, while the low-speed interface 1212 manages lower bandwidth-intensive operations. Such allocation of functions is an example only. In some implementations, the high-speed interface 1208 is coupled to the memory 1204, the display 1216 (e.g., through a graphics processor or accelerator), and to the high-speed expansion ports 1210, which may accept various expansion cards (not shown). In the implementation, the low-speed interface 1212 is coupled to the storage device 1206 and the low-speed expansion port 1214. The low-speed expansion port 1214, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 1200 may be implemented in a number of different forms, as shown in the figure. For example, it may be implemented as a standard server 1220, or multiple times in a group of such servers. In addition, it may be implemented in a personal computer such as a laptop computer 1222. It may also be implemented as part of a rack server system 1224. Alternatively, components from the computing device 1200 may be combined with other components in a mobile device (not shown), such as a mobile computing device 1250. Each of such devices may contain one or more of the computing device 1200 and the mobile computing device 1250, and an entire system may be made up of multiple computing devices communicating with each other.

The mobile computing device 1250 includes a processor 1252, a memory 1264, an input/output device such as a display 1254, a communication interface 1266, and a transceiver 1268, among other components. The mobile computing device 1250 may also be provided with a storage device, such as a micro-drive or other device, to provide additional storage. Each of the processor 1252, the memory 1264, the display 1254, the communication interface 1266, and the transceiver 1268, are interconnected using various buses, and several of the components may be mounted on a common motherboard or in other manners as appropriate.

The processor 1252 can execute instructions within the mobile computing device 1250, including instructions stored in the memory 1264. The processor 1252 may be implemented as a chipset of chips that include separate and multiple analog and digital processors. The processor 1252 may provide, for example, for coordination of the other components of the mobile computing device 1250, such as control of user interfaces, applications run by the mobile computing device 1250, and wireless communication by the mobile computing device 1250.

The processor 1252 may communicate with a user through a control interface 1258 and a display interface 1256 coupled to the display 1254. The display 1254 may be, for example, a TFT (Thin-Film-Transistor Liquid Crystal Display) display or an OLED (Organic Light Emitting Diode) display, or other appropriate display technology. The display interface 1256 may comprise appropriate circuitry for driving the display 1254 to present graphical and other information to a user. The control interface 1258 may receive commands from a user and convert them for submission to the processor 1252. In addition, an external interface 1262 may provide communication with the processor 1252, so as to enable near area communication of the mobile computing device 1250 with other devices. The external interface 1262 may provide, for example, for wired communication in some implementations, or for wireless communication in other implementations, and multiple interfaces may also be used.

The memory 1264 stores information within the mobile computing device 1250. The memory 1264 can be implemented as one or more of a computer-readable medium or media, a volatile memory unit or units, or a non-volatile memory unit or units. An expansion memory 1274 may also be provided and connected to the mobile computing device 1250 through an expansion interface 1272, which may include, for example, a SIMM (Single In Line Memory Module) card interface. The expansion memory 1274 may provide extra storage space for the mobile computing device 1250, or may also store applications or other information for the mobile computing device 1250. Specifically, the expansion memory 1274 may include instructions to carry out or supplement the processes described above, and may include secure information also. Thus, for example, the expansion memory 1274 may be provided as a security module for the mobile computing device 1250, and may be programmed with instructions that permit secure use of the mobile computing device 1250. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier and, when executed by one or more processing devices (for example, processor 1252), perform one or more methods, such as those described above. The instructions can also be stored by one or more storage devices, such as one or more computer- or machine-readable mediums (for example, the memory 1264, the expansion memory 1274, or memory on the processor 1252). In some implementations, the instructions can be received in a propagated signal, for example, over the transceiver 1268 or the external interface 1262.

The mobile computing device 1250 may communicate wirelessly through the communication interface 1266, which may include digital signal processing circuitry where necessary. The communication interface 1266 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access). TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 1268 using a radio-frequency. In addition, short-range communication may occur, such as using a Bluetooth®, Wi-Fi™, or other such transceiver (not shown). In addition, a GPS (Global Positioning System) receiver module 1270 may provide additional navigation- and location-related wireless data to the mobile computing device 1250, which may be used as appropriate by applications running on the mobile computing device 1250.

The mobile computing device 1250 may also communicate audibly using an audio codec 1260, which may receive spoken information from a user and convert it to usable digital information. The audio codec 1260 may likewise generate audible sound for a user, such as through a speaker, e.g., in a handset of the mobile computing device 1250. Such sound may include sound from voice telephone calls, may include recorded sound (e.g., voice messages, music files, etc.) and may also include sound generated by applications operating on the mobile computing device 1250.

The mobile computing device 1250 may be implemented in a number of different forms, as shown in the figure. For example, it may be implemented as a cellular telephone 1280. It may also be implemented as part of a smart-phone 1282, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

### VI. Sequences

| # | SEQUENCE | ANNOTATION |
|---|---|---|
| 1 | QLLFNKTKSVEFTFCNDTVVIPCFVTNMEAQNTTEVYVKWKF | Human CD47; aa |
| | KGRDIYTFDGALNKSTVPTDFSSAKIEVSQLLKGDASLKMDKS | 19-323 |
| | DAVSHTGNYTCEVTELTREGETIIELKYRVVSWFSPNENILIVIF | |
| | PIFAILLFWGQFGIKTLKYRSGGMDEKTIALLVAGLVITVIVIVG | |
| | AILFVPGEYSLKNATGLGLIVTSTGILILLHYYVFSTAIGLTSFVI | |
| | AILVIQVIAYILAVVGLSLCIAACIPMHGPLLISGLSILALAQLLG | |
| | LVYMKFVASNQKTIQPPRKAVEEPLNAFKESKGMMNDE | |
| 2 | MWPLVAALLLGSACCGSAQLLFNKTKSVEFTFCNDTVVIPCFV | Human CD47; aa |
| | TNMEAQNTTEVYVKWKFKGRDIYTFDGALNKSTVPTDFSSAKI | 1-323 |
| | EVSQLLKGDASLKMDKSDAVSHTGNYTCEVTELTREGETIIEL | |
| | KYRVVSWFSPNENILIVIFPIFAILLFWGQFGIKTLKYRSGGMDE | |
| | KTIALLVAGLVITVIVIVGAILFVPGEYSLKNATGLGLIVTSTGIL | |
| | ILLHYYVFSTAIGLTSFVIAILVIQVIAYILAVVGLSLCIAACIPM | |
| | HGPLLISGLSILALAQLLGLVYMKFVASNQKTIQPPRKAVEEPL | |
| | NAFKESKGMMNDE | |
| 3 | CEEPPTFEAMELIGKPKPYYEIGERVDYKCKKGYFYIPPLATHTI | Human CD46, aa |
| | CDRNHTWLPVSDDACYRETCPYIRDPLNGQAVPANGTYEFGY | 35-392 |
| | QMHFICNEGYYLIGEEILYCELKGSVAIWSGKPPICEKVLCTPPP | |
| | KIKNGKHTFSEVEVFEYLDAVTYSCDPAPGPDPFSLIGESTIYCG | |
| | DNSVWSRAAPECKVVKCRFPVVENGKQISGFGKKFYYKATVM | |
| | FECDKGFYLDGSDTIVCDSNSTWDPPVPKCLKVLPPSSTKPPAL | |
| | SHSVSTSSTTKSPASSASGPRPTYKPPVSNYPGYPKPEEGILDSL | |
| | DVWVIAVIVIAIVVGVAVICVVPYRYLQRRKKKGTYLTDETHR | |
| | EVKFTSL | |
| 4 | MEPPGRRECPFPSWRFPGLLLAAMVLLLYSFSDACEEPPTFEA | Human CD46; aa |
| | MELIGKPKPYYEIGERVDYKCKKGYFYIPPLATHTICDRNHTW | 1-392 |
| | LPVSDDACYRETCPYIRDPLNGQAVPANGTYEFGYQMHFICNE | |
| | GYYLIGEEILYCELKGSVAIWSGKPPICEKVLCTPPPKIKNGKHT | |
| | FSEVEVFEYLDAVTYSCDPAPGPDPFSLIGESTIYCGDNSVWSR | |
| | AAPECKVVKCRFPVVENGKQISGFGKKFYYKATVMFECDKGF | |
| | YLDGSDTIVCDSNSTWDPPVPKCLKVLPPSSTKPPALSHSVSTS | |
| | STTKSPASSASGPRPTYKPPVSNYPGYPKPEEGILDSLDVWVIA | |
| | VIVIAIVVGVAVICVVPYRYLQRRKKKGTYLTDETHREVKFTSL | |
| 5 | LQCYNCPNPTADCKTAVNCSSDFDACLITKAGLQVYNKCWKF | Human CD59; aa |
| | EHCNFNDVTTRLRENELTYYCCKKDLCNFNEQLENGGTSLSEK | 26-128 |
| | TVLLLVTPFLAAAWSLHP | |
| 6 | MGIQGGSVLFGLLLVLAVFCHSGHSLQCYNCPNPTADCKTAV | Human CD59; aa |
| | NCSSDFDACLITKAGLQVYNKCWKFEHCNFNDVTTRLRENEL | 1-128 |
| | TYYCCKKDLCNFNEQLENGGTSLSEKTVLLLVTPFLAAAWSLH | |
| | P | |
| 7 | LQCYNCPNPTADCKTAVNCSSDFDACLITKAGLQVYNKCWKF | Mature Human |
| | EHCNFNDVTTRLRENELTYYCCKKDLCNFNEQLEN | CD59; aa 26-102 |
| 8 | DCGLPPDVPNAQPALEGRTSFPEDTVITYKCEESFVKIPGEKDS | Human CD55; aa |
| | VICLKGSQWSDIEEFCNRSCEVPTRLNSASLKQPYITQNYFPVG | 35-381 |
| | TVVEYECRPGYRREPSLSPKLTCLQNLKWSTAVEFCKKKSCPN | |
| | PGEIRNGQIDVPGGILFGATISFSCNTGYKLFGSTSSFCLISGSSV | |
| | QWSDPLPECREIYCPAPPQIDNGIIQGERDHYGYRQSVTYACNK | |
| | GFTMIGEHSIYCTVNNDEGEWSGPPPECRGKSLTSKVPPTVQKP | |
| | TTVNVPTTEVSPTSQKTTTKTTTPNAQATRSTPVSRTTKHFHET | |
| | TPNKGSGTTSGTTRLLSGHTCFTLTGLLGTLVTMGLLT | |
| 9 | MTVARPSVPAALPLLGELPRLLLLVLLCLPAVWGDCGLPPDVP | Human CD55; aa |
| | NAQPALEGRTSFPEDTVITYKCEESFVKIPGEKDSVICLKGSQW | 1-381 |
| | SDIEEFCNRSCEVPTRLNSASLKQPYITQNYFPVGTVVEYECRP | |
| | GYRREPSLSPKLTCLQNLKWSTAVEFCKKKSCPNPGEIRNGQID | |
| | VPGGILFGATISFSCNTGYKLFGSTSSFCLISGSSVQWSDPLPEC | |
| | REIYCPAPPQIDNGIIQGERDHYGYRQSVTY ACNKGFTMIGEHS | |
| | IYCTVNNDEGEWSGPPPECRGKSLTSKVPPTVQKPTTVNVPTTE | |
| | VSPTSQKTTTKTTTPNAQATRSTPVSRTTKHFHETTPNKGSGTT | |
| | SGTTRLLSGHTCFTLTGLLGTLVTMGLLT | |
| 10 | DCGLPPDVPNAQPALEGRTSFPEDTVITYKCEESFVKIPGEKDS | Maturc aa 35-353 |
| | VICLKGSQWSDIEEFCNRSCEVPTRLNSASLKQPYITQNYFPVG | CD55; Human |
| | TVVEYECRPGYRREPSLSPKLTCLQNLKWSTAVEFCKKKSCPN | |
| | PGEIRNGQIDVPGGILFGATISFSCNTGYKLFGSTSSFCLISGSSV | |
| | QWSDPLPECREIYCPAPPQIDNGIIQGERDHYGYRQSVTYACNK | |
| | GFTMIGEHSIYCTVNNDEGEWSGPPPECRGKSLTSKVPPTVQKP | |
| | TTVNVPTTEVSPTSQKTTTKTTTPNAQATRSTPVSRTTKHFHET | |
| | TPNKGSGTTS | |
| 11 | EGRGSLLTCGDVEENPGP | T2A |
| 12 | LEGGGEGRGSLLTCGDVEENPGPR | T2A |
| 13 | GSGATNFSLLKQAGDVEENPGP | P2A |
| 14 | ATNFSLLKQAGDVEENPGP | P2A |
| 15 | QCTNYALLKLAGDVESNPGP | E2A |
| 16 | VKQTLNFDLLKLAGDVESNPGP | F2A |
| 17 | GSGEGRGSLLTCGDVEENPGP | T2A |
| 18 | AAGSGEGRGSLLTCGDVEENPGP | T2A |
| 19 | GUUUUAGAGCUA | Exemplary |
| | | crRNA repeat |
| 20 | UAGCAAGUUAAAAUAAGGCUAGUCCGUUAUCAACUU | Exemplary |
| | GAAAAAGUGGCACCGAGUCGGUGCUUU | tracrRNA |
| | GAAA | Tetraloop |
| | | sequence |

### EXAMPLES

The following examples are provided so as to describe to the skilled artisan how to make and use methods and compositions described herein; and are not intended to limit the scope of the present disclosure.

### Example 1—Incucyte

Human T cells from healthy donors were obtained by leukapheresis. Allogeneic HIP CAR T cells were generated via 0.2× or 1× scale manufacturing process. Gene editing was used to delete B2M, CIITA and TCRa expression and viral transduction was used to overexpress CD47 and CD19CAR. Generated CAR T cells were evaluated in vitro for serial tumor killing during repetitive tumor challenges (4×) and long-term expansion over 14-day period using the Incucyte platform. In this assay, target cells (Naml-6:iRFP or Raji:iRFP) were seeded in flat-well plate format at and CAR-T cells were seeded at 1:1, 1:2, 1:4,1:8, 1:16 E:T ratio (adjuster to CAR+ T cell number). After initial 5-day stimulation, a portion of the culture was transferred to the new plate containing target cells for 3 day restimulation. This step was repeated 3 consecutive times. T cell and tumor cell proliferative index was quantified as the geometric mean over each re-stimulation at 1:8 E:T. T cell durability of growth and response are quantified as slope of the fold change of restimulation for T or target cell growth respectively at 1:8 E:T.

During serial tumor challenge, donor-to-donor variability was observed at 1:8 E:T ratio. The average growth rate of T cells at 1:8 E:T was shown to be 4-fold during each restimulation cycle, with several donors that exceed this value (at 10% increase or more). Average target cell growth rate at 1:8 E:T is 0.2-fold. Average durability of T cell growth is −1, while more durable donors show increased durability at 0 or higher. Average durability of response is 1, with better performing CAR T cells having value of 0 or less.

### Example 2—MesoScale Discovery (MSD)

Bulk cytokine measurement was performed using MesoScale Discovery (MSD) analysis, 10 cytokines were measured (GM-CSF, GzmA, GzmB, IFNg, TNFa, 112, I16, 1117A, I11b, I11RA). Target cells (Naml-6:iRFP) were seeded in flat-well plate format, and CAR-T cells were seeded at 1:1, 1:2, 1:4, 1:8, 1:16 E:T ratio (adjusted to CAR+ T cell number). Co-culture supernatant was collected 48 hours after initial seeding. Cytokine production was quantified as production per cell(pg/cell) at 1:2 E:T.

Bulk cytokine measurement showed average 1.5 μg cytokine production per T cell, with several donor-derived CAR T cells producing 1.9 μg/cell or higher (25% or more above average).

### Example 3—Isoplexis

Cytokine profiling on the single cell levels were evaluated using the Isoplexis Human Adaptive Immune assay. CAR-T cells were co-cultured with Nalm-6 target cells in U-bottom plates, 48 hrs after co-culture, cells were collected. CD4 and CD8 T cells were isolated via positive selection, stained for CD4 or CD8 and loaded on the IsoCode chips as suggested per manufacturer. Under Isoplexis analysis, two metrics were employed: a polyfunctionality strength index (PSI) was defined as signal intensity of the cytokine production by T cell population that produces 2+ cytokines, and a multi-functionality index was defined as the percentage of T cells that produces 4+ cytokines.

A median PSI value of 200 was observed for the CD8 T cell population, and 90 for the CD4 T cell population. Multifunctionality of 0.3-3% is observed in CD8 T cell population, and 0.1-2% in CD4 T cell population.

### Example 4—nCounter

Gene expression profiling was performed on pre-production (APH), post-CAR manufacturing (resting), early activated (2 day post-tumor challenge), and exhausted (post serial tumor challenge) CD4 and CD8 T cells using the CAR T panel for the nCounter Sprint profiler (NanoString Technologies). For the pre-production and post-CAR manufacturing resting conditions, the cells were thawed in presence of 112. CD4 and CD8 T cells were isolated via positive selection, and total mRNA was isolated in triplicates. To generate early activated and exhausted CAR T cell subsets, target cells (Naml-6) were seeded in 6 flat-well plate format, and CAR-T cells were seeded to generate a 1:8 E:T ratio. Serial restimulation was performed as follows: the cultures were kept in the incubator for 5 days. Three restimulations are performed on d5, d8 and d11, 2 days after initial seeding, co-cultured cells were isolated for the early activation timepoint. Co-cultured cells at the end of the assay (14 days) represent exhausted T cell timepoint. CD4 and CD8 cells were collected via positive selection.

Differential gene expression of T cells was performed where donor cells that scored higher across the assays (IncuCyte, MesoScale cytokine, in vivo assays) were compared to the rest of the pool (good or poor designated donors). Gene expression profiling identified that better performing donors are less activated and have a less NK-like signature at the pre-production state. Additionally, better performing donors have an activated phenotype at resting CAR T state. CAR T cells derived with increased functionality skew towards Th1/Tc1 and Th17/Tc17 state at the resting and activated stages. Better performing donors show higher amount of mucosal associated invariant T (MAIT) cells at the pre-production, resting, and early activation steps.

### Example 5—In Vivo

In vivo efficacy was evaluated using a systemic NALM-6 tumor model on a NSG mouse background. Tumor cells were injected at. 3 days before CAR T cell injections. Efficacy of response was evaluated at high (5e+6 CAR+) or low (0.5e+6 CAR+) T cell dose. In vivo response was calculated as area under the curve (AUC) of the target cell bioluminescent flux.

During in vivo challenge, a median area under the curve (AUC) value of 29 was observed at high dose of CAR T, and AUC value of 10548 was observed at low dose of CAR T.

### Example 6—HIP CAR T Persistence Study in Humanized Mice

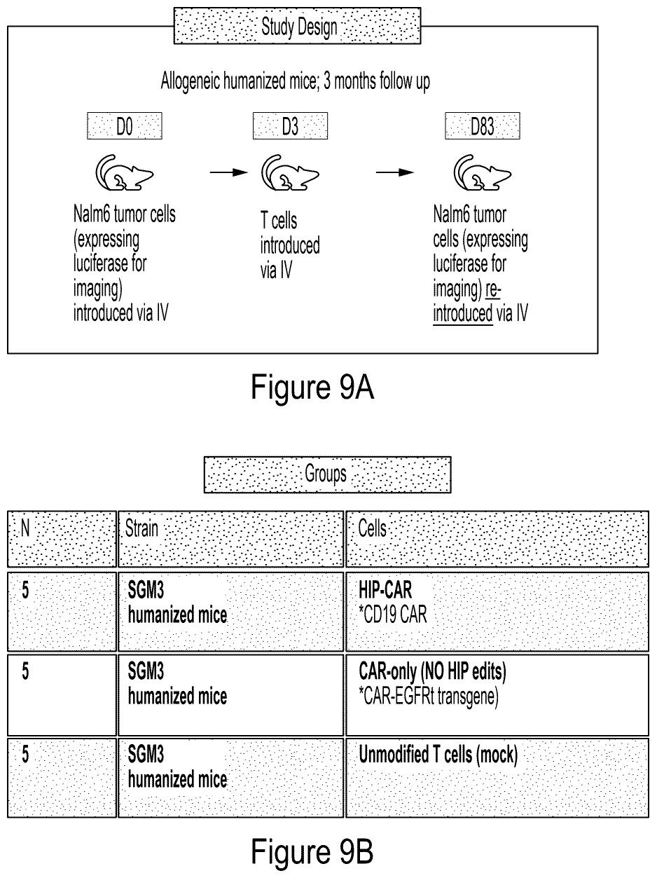

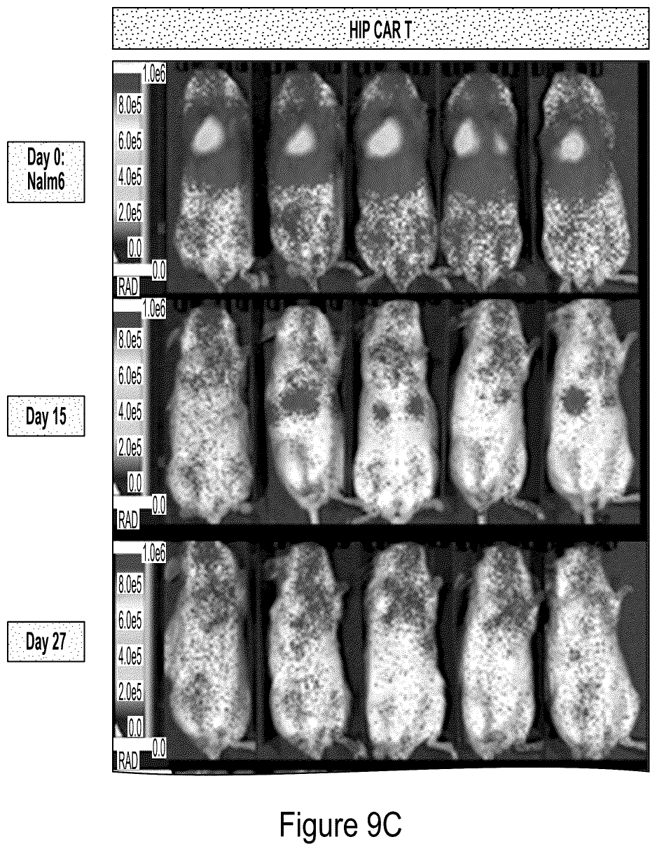

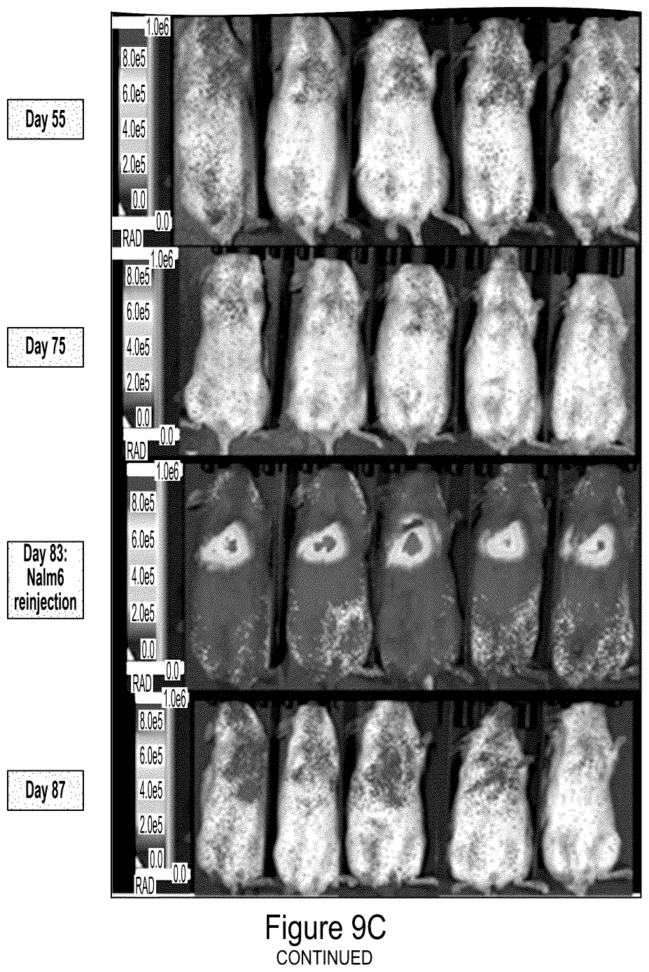

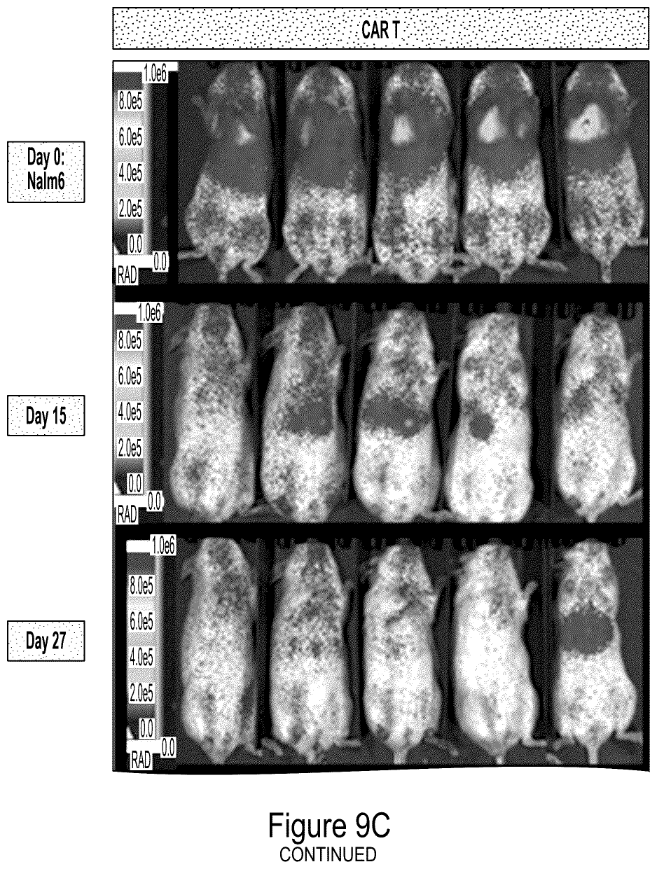

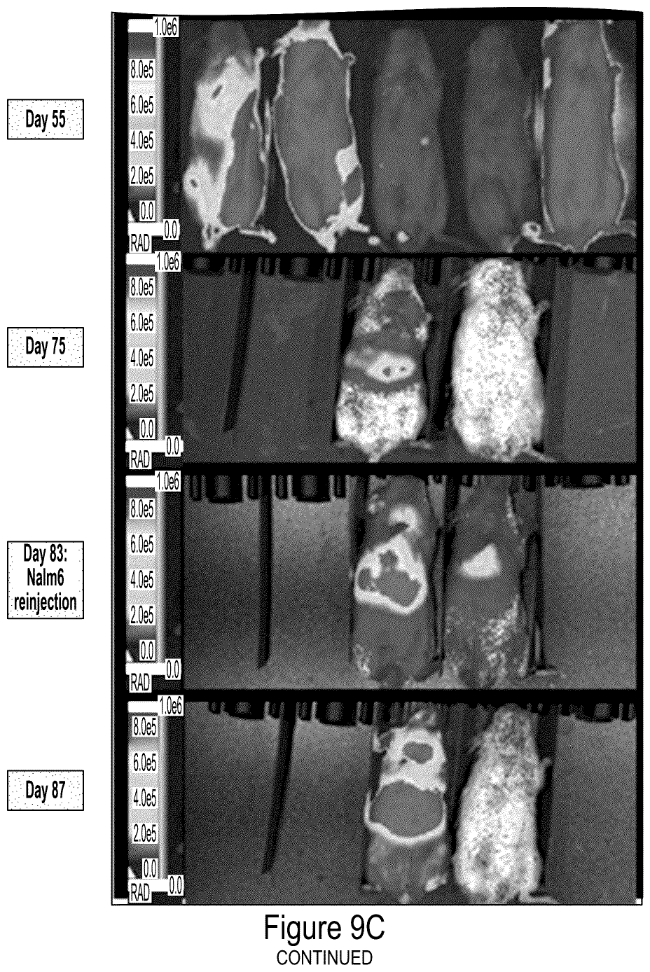

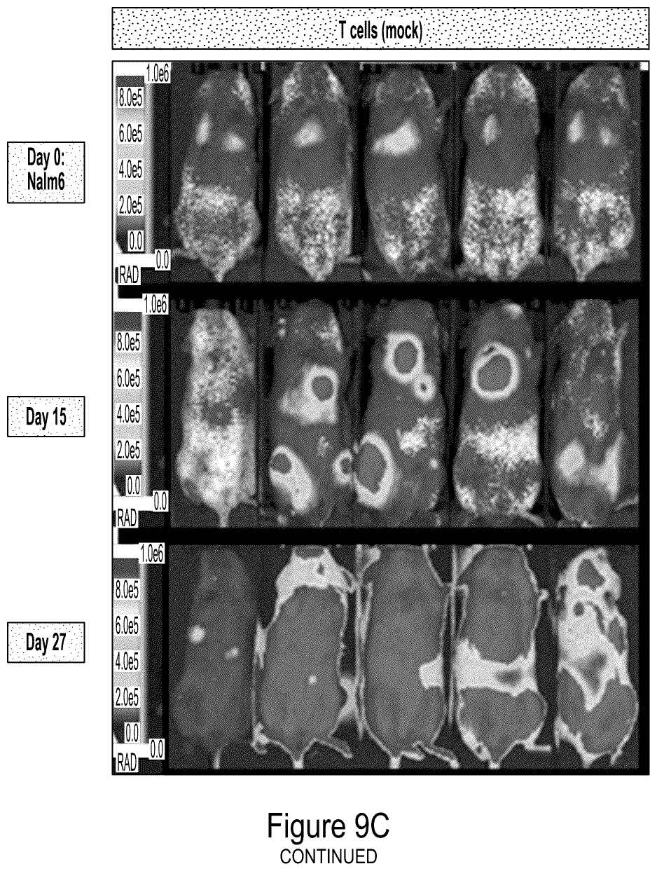

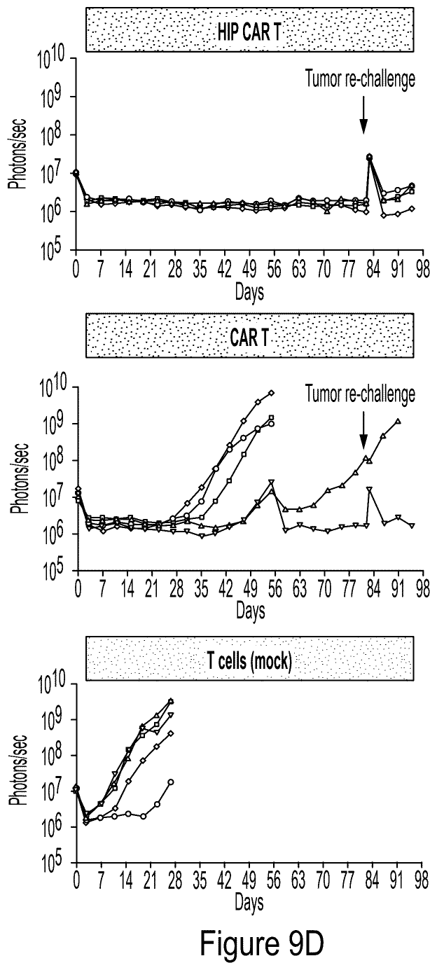

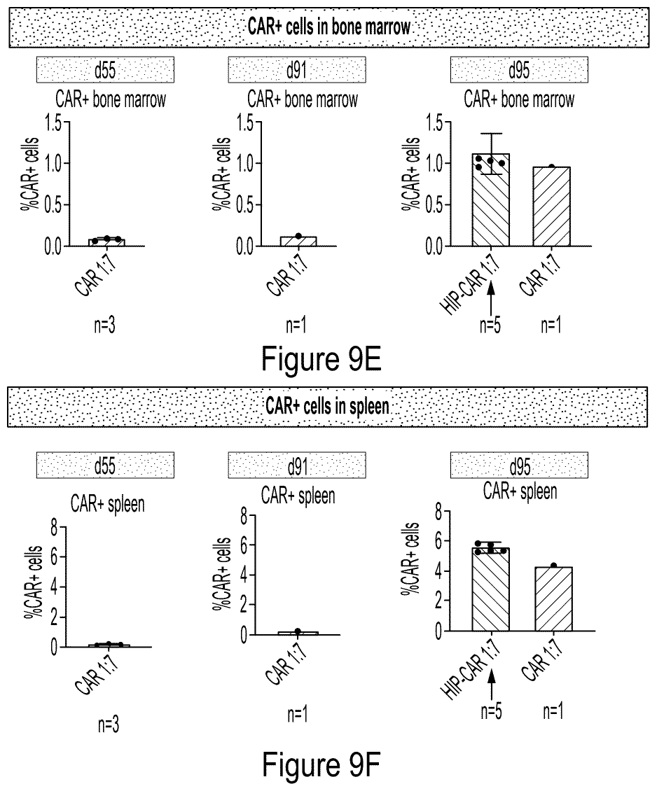

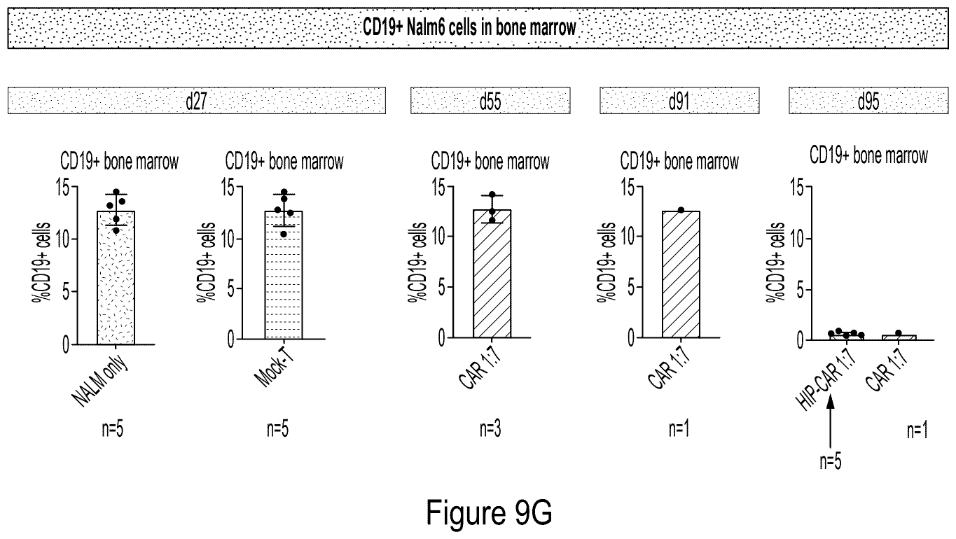

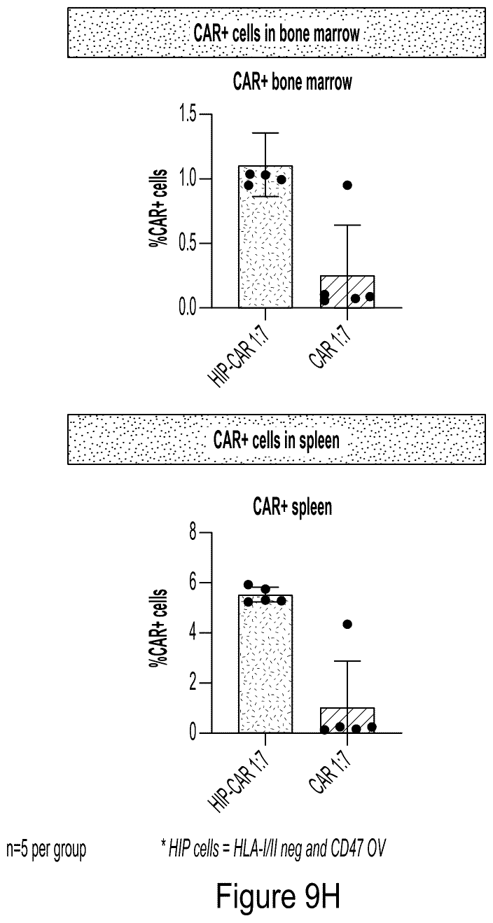

The persistence of HIP CAR T (from primary T cells), CAR T cells and unmodified T cells were analyzed in humanized mice. At day 0, Nalm6 tumor cells (expressing luciferase for imaging) were introduced into humanized mice. At day 3. HIP CAR T cells (CD19 CAR), CAR T cells (CAR-EGFRt transgene) with no HIP edits and T cells (unmodified) were introduced. At day 83, Nalm6 tumor cells (expressing luciferase for imaging) were reintroduced (see FIG. 9A). The three groups of cells analyzed were as set out in FIG. 9B: HIP CAR T cells (CD19 CAR), CAR T cells (CAR-EGFRt transgene) with no HIP edits and T cells (unmodified). The HIP CAR T cells were those derived from donor 6. 5 mice in each category were analyzed.

In vivo bioluminescent imaging was used to assess the presence of tumor cells in injected mice at day 0, 15, 27, 55, 75, 83 and 87 (unless the mice had already been sacrificed due to tumor growth)(FIGS. 9C and 9D). It was observed that by day 75, the mice treated with HIP CAR-T eliminate the tumor cells suggesting that tumor cells are being eliminated by the HIP CAR T and that the HIP CAR-T cells must therefore be present (and are not being rejected by the host immune system). Comparatively, the CAR T cells (-HIP edits) initially kill the tumor (day 27) but are eventually rejected by the host immune system such that the tumor cells can grow (day 55). At day 75, some CAR T (-HIP edits)-injected mice have been sacrificed due to tumor growth. Mock-injected mice (i.e. T cell only) are not able to eliminate tumor.

Upon rechallenge by tumor (day 83). CAR T (-HIP edits)-injected mice are not able to eliminate tumor and by day 87, the tumor cells are beginning to expand. In contrast, in HIP CAR-T-injected mice the tumor is eliminated, suggesting that the HIP CAR-T cells have survived and are functional.

To assess the presence of CAR T/HIP CAR T cells or tumor cells in these mice at different time points, samples were collected from the mice at a single time point when they were sacrificed due to tumor growth (at day 27, 55, and 91) or at the end of the experiment (at day 95). All 5 animals injected with HIP CAR T survived to day 95, compared to only one animal with CAR T (-HIP) (others needed to be sacrificed sooner due to cancer growth).

Flow cell analysis was carried out on in vivo blood samples. For the detection of tumor cells and injected CAR-T cells, recipient splenocytes and bone marrow cells were stained with Zombie live/dead stain (77184, Biolegend), anti-CD19 (clone HIB19, Biolegend) and FMC63 (clone Y45, AcroBio Systems). Cells were measured by flow cytometry (BD Aria) and gated for live cells, followed by CD19+ cells or CAR+ cells. Results were expressed as percentage of positive population compared to control samples. In addition, recipient splenocytes and bone marrow cells of the HIP CAR-T group were stained with anti-HLA-A,B,C antibody (clone G46_2.6,BD Biosciences), anti-HLA-DR,DP,DQ antibody (clone Tu3a, BD Biosciences) and anti-CD47 (clone B6H12. BD Biosciences) to identify HIP cells.

In the bone marrow (FIGS. 9E and 9H), very few CAR+ cells were seen in the CAR T (-HIP) sacrificed mice at day 55 and day 91. Comparatively, at day 95, many CAR+ cells are seen in HIP CAR T cell-injected mice. Similar results are seen in the spleen (FIGS. 9F and 9H). These data suggests that HIP CAR-T cells survive in both the bone marrow and spleen of humanized mice. FIG. 9H shows summary data from all timepoints.

In the bone marrow (FIGS. 9G and 9I), very few CD19+ tumor cells are seen in HIP CAR T cell-injected mice at day 95. Comparatively, the bone marrow of tumor only (Nalm only) and unmodified T cell (Mock-T) mice at day 27, as well as CAR T (-HIP) mice at day 55 and day 91 show far more tumor cells. These data suggest that HIP CAR-T cell-injected group of mice have significantly reduced Nalm6 tumor in the bone marrow. FIG. 9I shows summary data from all timepoints.

Bulk HIP CAR T cells survive in humanized mice (FIG. 13). While injected bulk HIP showed <50% HIP cells, on D95 all surviving CAR T cells have the HIP phenotype.

HIP CAR T cells show greater cell persistence than CAR T cells or T cells since HIP CAR T cells survive in humanized mice and show function even after re-injection (after 83 days).

### Example 7—Human HIP Cells are Well Suited as a Clinical Cell Product

Human primary pancreatic islets, engineered to a hypoimmune phenotype (i.e., HLA class I and II-negative and CD47-overexpressing phenotype), were generated and shown to survive, engraft and ameliorate diabetes in immunocompetent, allogenic, diabetic humanized mice. Unengineered human pseudo islets (p-islets) or HLA-I/II double knockout (DKO) human p-islets were used as controls.

The efficiency to generate fully engineered cells in a clinical setting will most likely be lower than in a preclinical setting and the clinical cell product could contain fractions of unengineered (wildtype) or DKO-edited (partially engineered) cells. To test how a contamination with wildtype (wt) or DKO (partially engineered) cells within the HIP p-islets affects the overall organoid survival and function, experiments were performed with 1:1 mixtures of cells.

### Methods

### Gene Editing of Human Primary Islets

Human primary cadaveric islets were purchased and cultured overnight in PIM media. CRIPSR technology was used for the disruption of the B2M and CIITA gene. Islet clusters were dissociated in single cells for 10 min at 37° C. Briefly, cells were transduced with a final concentration of 50M per mL in P3 buffer. 20 μL of the cell suspension were pipetted in one well of the 8-strip containing 13 ug Cas9 enzyme and 5 uM sgRNA, respectively. Islet cells were transferred in U-bottom 96-well plates containing 50,000 cells per well in PIM(S) media and rested for 1 h at 37° C., and 5% CO2 before moving the plate on to a shaker for islet re-clustering. Complete media change was performed after 48h, and islet clusters were incubated on the shaker for another 24h.

Islet clusters were dissociated again in single cells for cells sorting using the anti-HLA-A,B,C antibody or IgG1 isotype-matched control antibody and anti-HLA-DR,DP,DQ antibody or IgG2a isotype-matched control antibody. Double negative cells were sorted and replated in U-bottom 96-well plates as described above for islet re-clustering on the shaker. After 24h, islets were dissociated in single cells for CD47 and luciferase transduction. Spinfection was performed with the presence of 10 ug/ml protamine sulfate at 300g for 15 min. Cells were replated in U-bottom 96-well plates as described above for islet re-clustering on the shaker. After 48h, cells were dissociated in single cells and underwent cell sorting for human CD47 with anti-CD47 antibody or IgG1 isotype-matched control antibody. Luciferase expression was confirmed by adding D-luciferin. Signals were quantified in maximum photons s−1 cm−2 sr−1. Islet cells were replated in U-bottom 96-well plates as described above for islet re-clustering on the shaker until transplantation.

### Flow Cytometry

To assess HLA class I expression, single islet cells were dissociated and labeled with APC labeled anti-HLA-A,B,C antibody or APC-conjugated IgG1 isotype-matched control antibody. To assess HLA class II expression, cells were incubated with Alexa-flour647-labeled anti-HLA-DR,DP,DQ antibody or Alexa-flour647-labeled IgG2a isotype-matched control antibody. To assess CD47 expression, the PerCP-Cy5-conjugated anti-CD47 or PerCP-Cy5-conjugated IgG1 isotype-matched control antibody was used.

### Mouse Models

Humanized NSG-SGM3 mice were used. The number of animals per experimental group is presented in FIG. 14. Mice received humane care in compliance with the Use Committee and performed according to local guidelines. To induce diabetes, mice were injected with 60 mg/kg streptozotocin intraperitoneally (STZ) for 5 consecutive days. Islet clusters were injected intramuscularly into the hindlimb muscle with a 27 G needle on day 0. On day 0 and day 4, all mice received Simulect (anti-Human IL2RA/CD25 antibody) at a concentration of 0.25 mg/kg.

### In Vivo BLI Imaging

Mice were monitored on day 0, day 3 and every second day until day 13 and subsequently every 4 days until day 29. D-luciferin firefly potassium salt (375 mg/kg) was dissolved in sterile PBS and was injected intraperitoneally (250 μl per mouse) into anesthetized mice. Animals were imaged and ROI bioluminescence was quantified in units of maximum photons per second per centimeter square per steradian (p s−1 cm−2 sr−1). The maximum signal from an ROI was measured-.

### Results

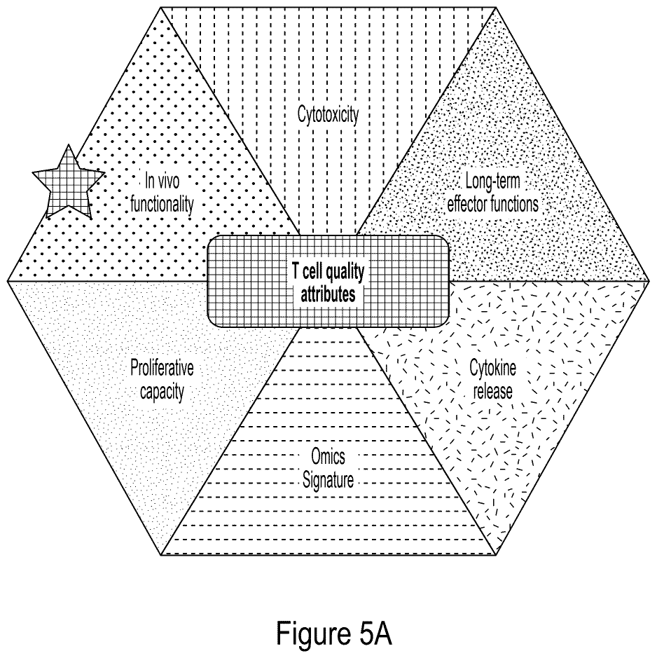

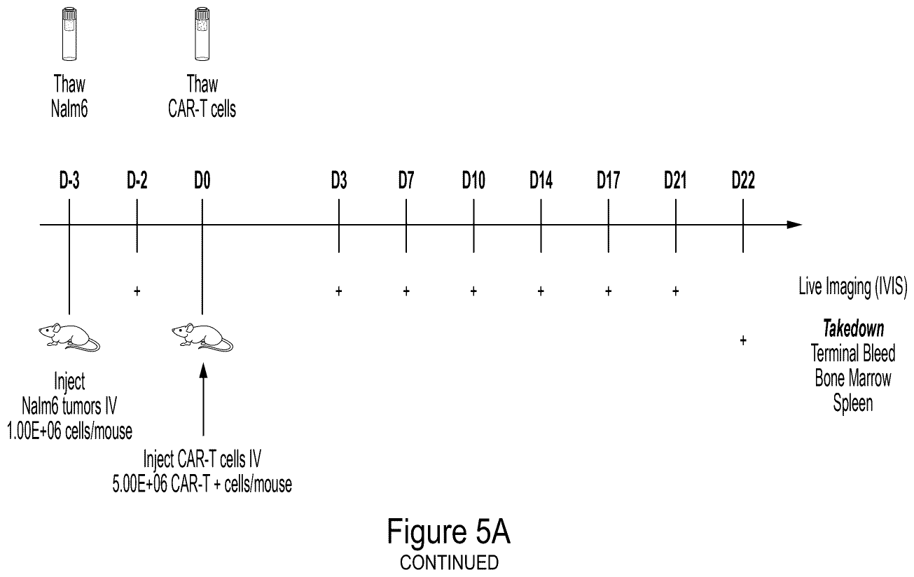

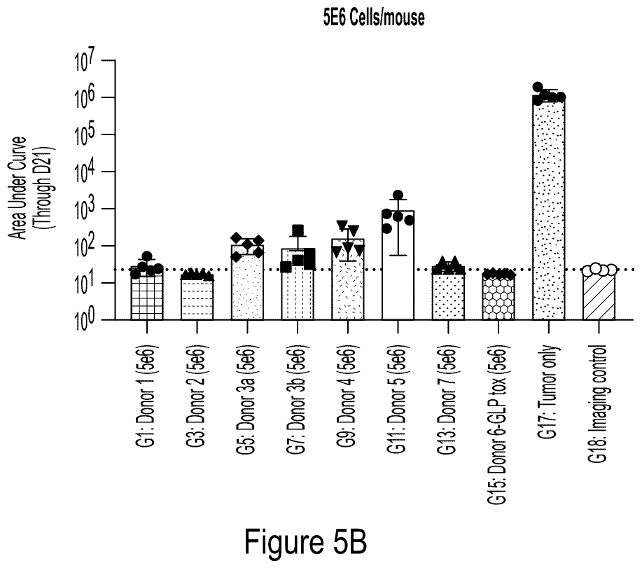

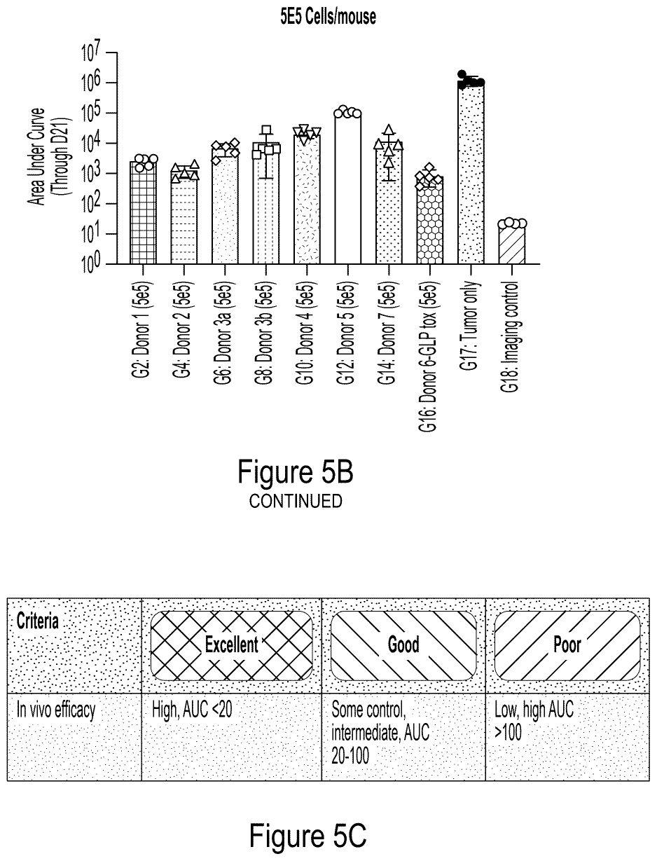

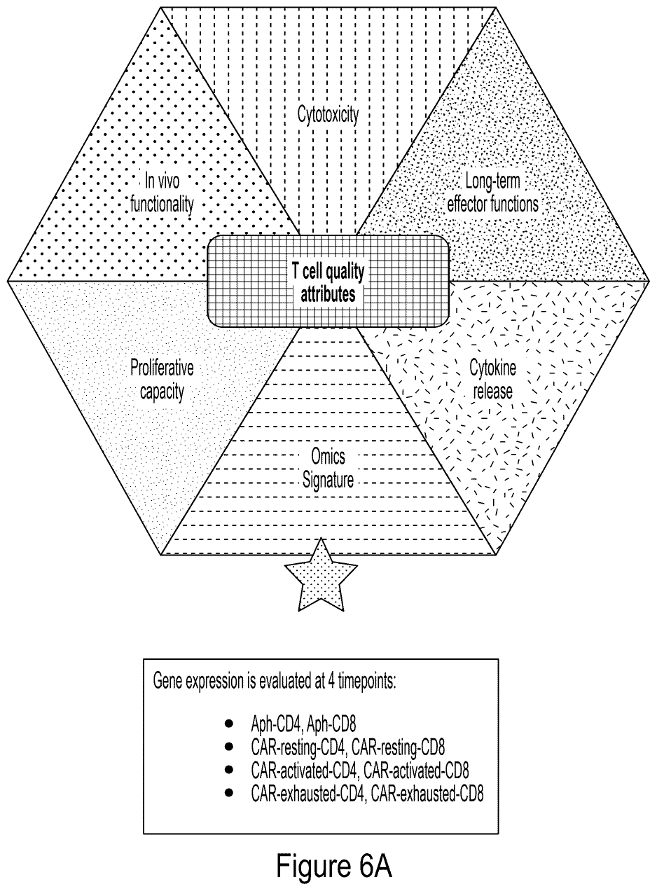

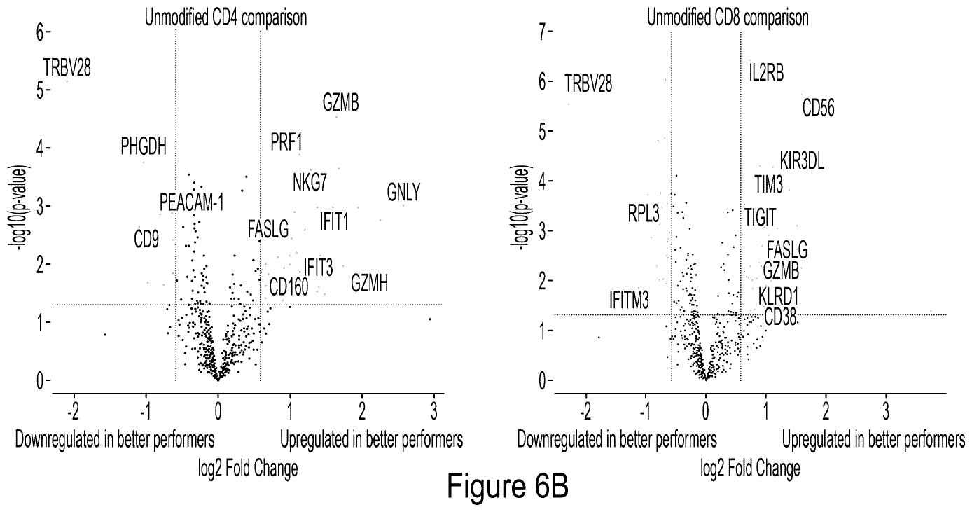

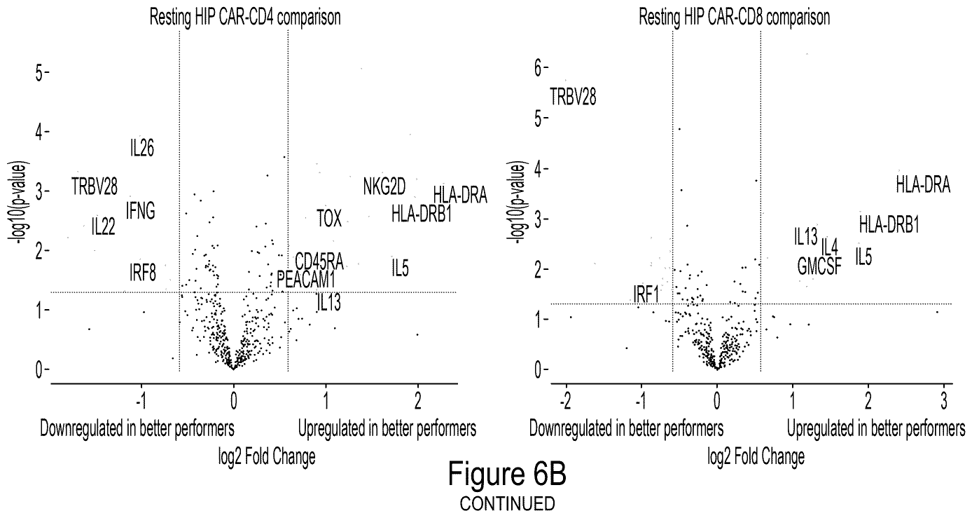

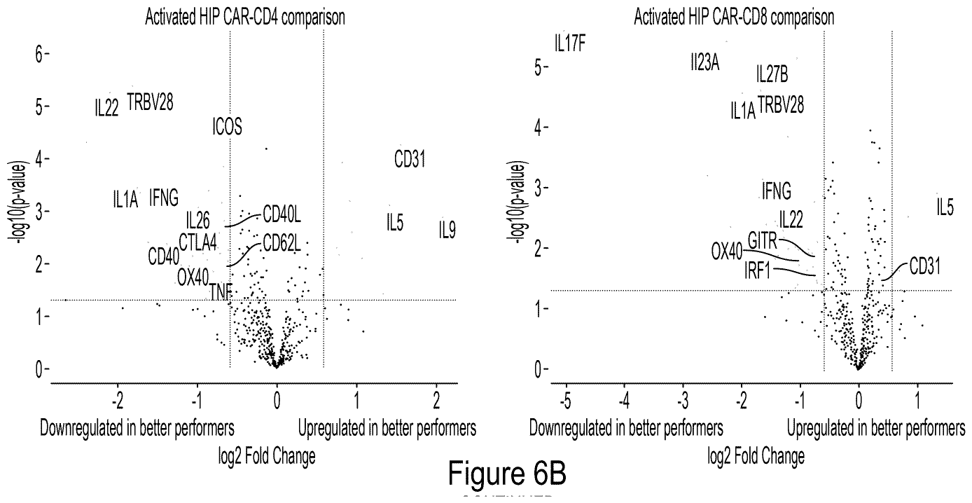

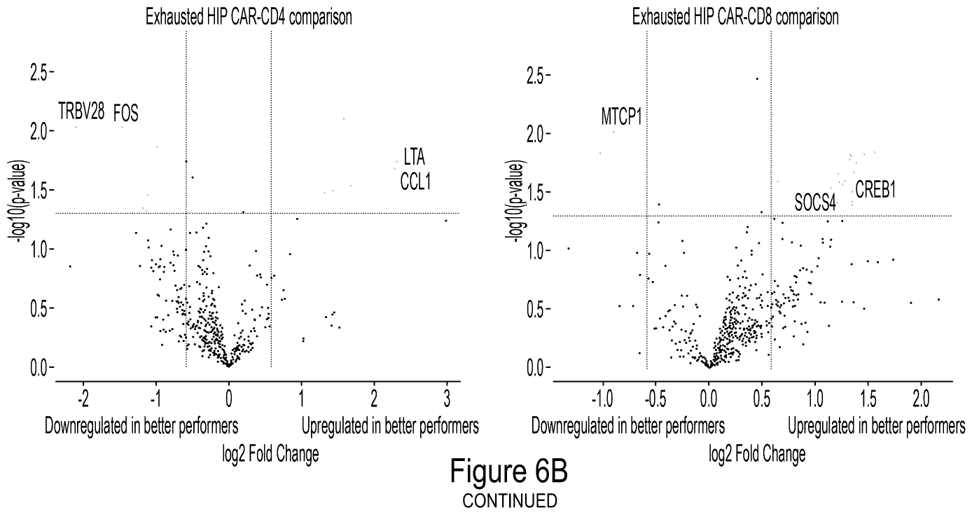

First, wt and HIP islet cells were reaggregated into p-islets and transplanted into allogeneic, diabetic humanized mice. When the Luc+ wt p-islet fraction was followed by BLI, they vanished over 10 days (FIG. 5a, b). Luc+ HIP p-islet cells, on the other hand, successfully engrafted and survived for the duration of the experiment (FIG. 5d, e). Glucose monitoring in both experiments showed normalization of blood glucose by the prevailing HIP p-islet fraction (FIG. 5c, f). Similarly, mixed DKO and HIP islets were reaggregated and transplanted into allogeneic, diabetic humanized mice. When the DKO fraction was labelled with Luc+, we observed rapid disappearance of Luc+ cells (FIG. 5g, h) while in the Luc+ HIP mixtures, the signal persisted for the duration of the experiment (FIG. 5j, k). Again, glucose monitoring in both experiments showed normalization of blood glucose by the prevailing HIP p-islet fraction (FIG. 5i, l). Together these results demonstrate that as long as there are enough HIP p-islet cells, contaminations with unengineered or partially engineered cells do not jeopardize the engraftment and function of the HIP p-islet grafts. These results surprisingly demonstrate that clinical cell therapy products containing HIP cells are functional even if the cell therapy products contain contaminations with other unedited or partially edited cells. The present inventors have found that cell therapy products containing HIP cells do not need to be completely pure in order to be functional.

### Example 8—T Cell Functional Assay Correlation Analysis

The selection of cell donors or a pool of cell donors is a critical element contributing to the success of cell therapy manufacturing and the generation of safe and effective cell therapy products. However, interpreting the mechanisms and principles that govern cell therapy activity and how the cell therapy products will work according to a specific parameter or parameters such as donor characteristics, cell quality, clinical outcomes, and the like is not fully understood. The activities or features of cells from a single donor or pool of donors, for example donor characteristics (e.g., donor age, BMI, blood type, etc), the cellular composition (e.g., cell subtypes, PBMC panel, CD4/CD8 subsets, etc.), target killing activity (e.g., short-term or long term cytolytic activity), gene expression, activation/exhaustion, cytokine production, hypoimmune progenitor (HIP) panel-editing, T cell/target cell proliferation, and the like can be identified as effective factors in predicting the efficacy of a cell therapy product.

This example demonstrates how donor cell attributes can be assessed via multidimensional analyses to define a group or groups of factors that predict cell quality and/or set up a range or cutoff values for specific cell parameters to support donor selection criteria. In vivo functionality, in vitro functionality, and donor cell characteristics (as described in Examples 1 to 6) were analyzed in relation to CAR T anti-tumor activity (FIG. 15a-15b). Specifically, the correlation among donor characteristics and tumor kinetic characteristics was explored using R Corr Package with the hierarchical clustering ordering method. The analysis revealed that donor age was one factor associated with CAR T cell functionality and tumor control. Specifically, the analysis demonstrated that donor age is associated with tumor control (e.g., cells derived from younger donors had improved ability to control tumor growth) and that younger donors (e.g., pre-menopausal females, for example ages 18-36) have higher frequencies of CD8+ T cells and a greater proportion of T cells that express markers associated with younger phenotypes. In addition, the analysis demonstrated that donor BMI is associated with tumor control (e.g., cells derived from donors with lower BMI had improved ability to control tumor growth). Moreover, CD27 expression in CAR T cells positively correlated with tumor control while CD69 expression and CD25 expression negatively correlated with tumor control. Further, the level of CD27 expression was associated with donor age. Taken together, the results demonstrate that cell parameters can prospectively identify donors, or pools of donors, for the generation of cell therapies with improved efficacy.

These results also demonstrate the utility of a multidimensional analysis for cell therapy donor selection. Additional parameters for use in such multidimensional analysis are described anywhere in the present application and include donor background and patient background (e.g., genomic background, transcriptomic background, proteomic background, cancer specific genome background, tumor microenvironment background, and the like).

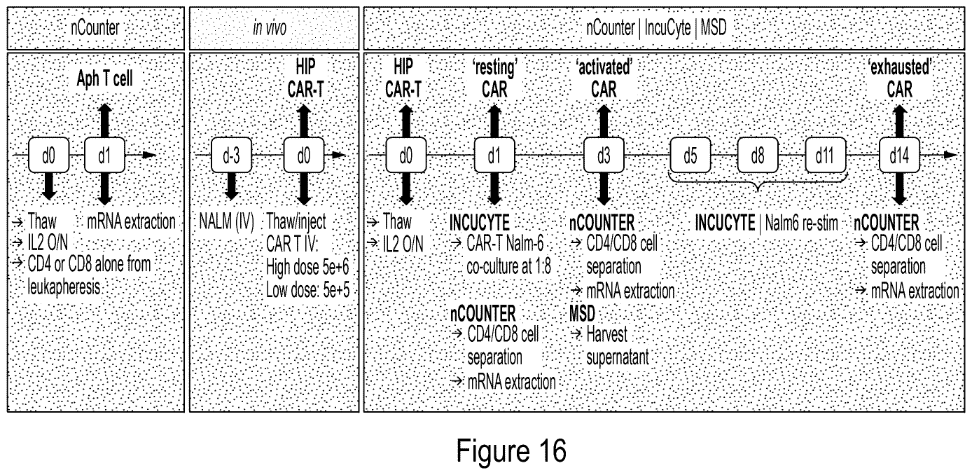

FIG. 16 shows a schematic diagram of potential assays that can be used to evaluate CAR T cell pre-clinical functionality. Assays such as nCounter, in vivo NALM tumor cell xenograft assays, IncuCyte, and Meso Scale Discovery (MSD) cytokine assays can produce single, quantitative indices to enable classification of CAR T cells based on functional performance.

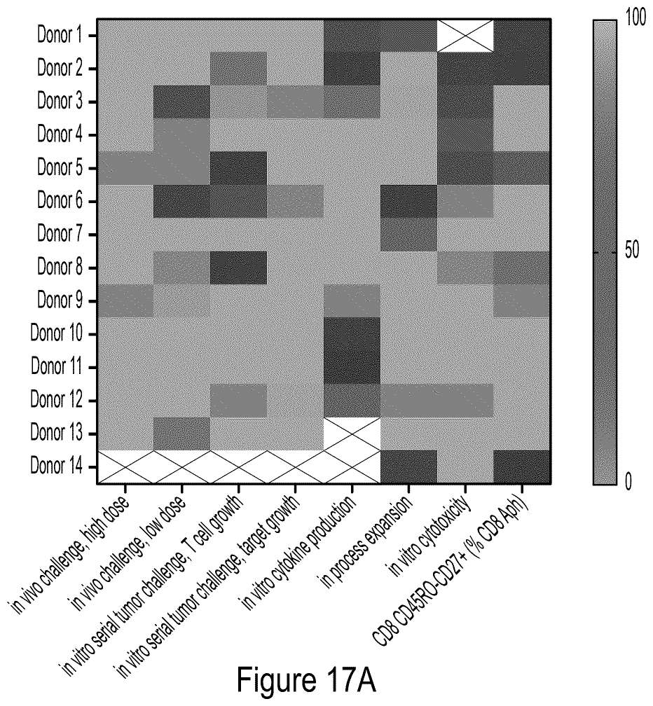

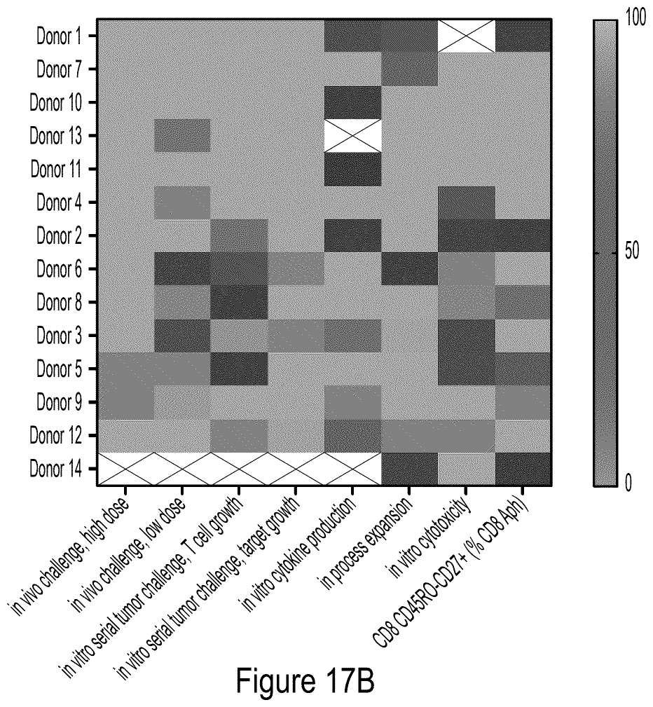

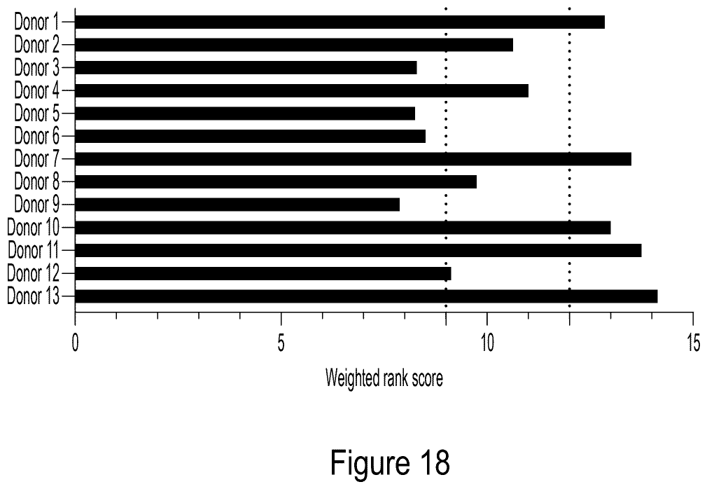

As shown in FIGS. 17a and 17b, HIP CAR-T cells can be assayed to determine a level, signal, or score for various cell parameters. A compilation (e.g., a heat map) of various levels, signals, or scores can be produced and used to characterize donor samples that have attributes correlating with sufficient (or better) pre-clinical performance of HIP CAR-T cells. For example, donor sample performance for a particular parameter is set at 0 if the measured value is the lowest within the group (poor performance for that parameter), and is set at 100 if the value is above average (excellent performance for that parameter). Donors can then be ranked as excellent, good, or poor. Excellent donors do not have low performance for any functional parameters; good donors fail in one functional parameter; and poor donors fail in two or more functional parameters. FIG. 18 shows an exemplary ranking of weighted performance values of 13 T cell donors. Donor performance was rank-scored using a 1 to 5 point scale from failing to perform (score of 1) to exceeding performance (score of 5) for a described parameter. Functional assays and known phenotype parameters of success were also scored at 1 (least predictive of performance) to 5 (most predictive of performance).

Cell samples (e.g., donor cell samples, e.g., donor cell samples comprising T cells) can be assessed at various stages of a cell therapy product (e.g., T cell therapy product). An exemplary stage is the blood draw stage. At this stage, a donor cell sample can be blood (e.g., whole blood). Parameters, such as CD4:CD8 ratio, NK cell %, Monocytes/HLA-DR+, iNKT, CD101, and/or CD39, can be assessed. For example, lymphocyte counts are collected using complete blood count equipment-Sysmex. To assess detailed distribution of PBMC subsets, T cell subsets, and exhaustion markers, antibody staining using various panels set out in Table 21 can be performed. Antibody mix is added to 200ul of whole blood, and samples are let to stand at RT for 15 min in the dark. 2 ml of red blood cell lyse buffer containing fixative solution is added, and incubated in the dark for 15 min, spun down, and samples are resuspended in 100ul of lyse/fix butter, and run on flow cytometer. In order to quantify CD3/CD4/CD8 cell counts through flow cytometry, quantification beads are added to the quantification panel. Alternatively, 50ul of whole blood is stained with quantification panel for 15 min in dark, and 50ul of quantification beads and 450 ul of red blood cell lysis/fixative buffer is added. Samples are run on flow cytometer after 15 min of incubation.

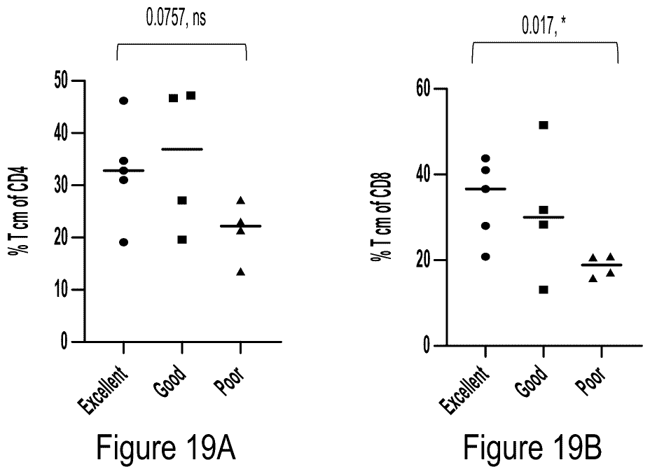

The distribution of T cell subsets can be assessed from a blood sample (e.g., a blood draw-prescreen). As shown in FIGS. 19A and 19B, higher percentages of central memory (Tcm) CD4 and CD8 T cells may correlate with improved performance of a fully-edited HIP CAR-T cell product.

Cell samples can also be assessed at a stage following leukapheresis. For instance, assays may be performed on aphresis material (e.g., an enriched white blood cell sample) to characterize T cell parameters including T cell proliferation and caspase 3 activity. Briefly, Stage 1 samples are thawed, and 0.5e+6 cells are transferred into 96 well plate, washed, sequentially stained with viability stain and indicated panels (PBMC panel, T cell subset panel, active caspase-3 panel. T cell activation and T cell exhaustion panel), washed and then run on the flow cytometer.

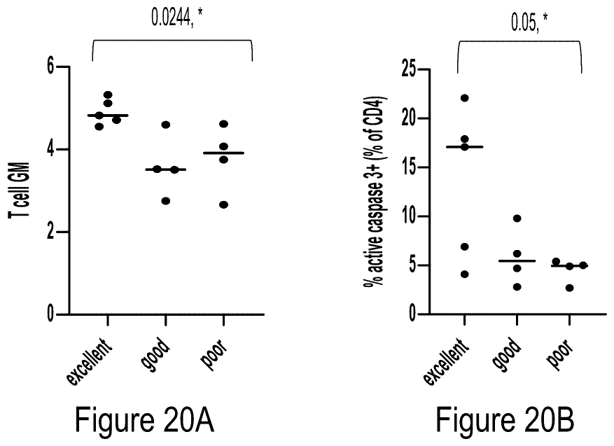

FIG. 20A shows that higher proliferation of T cells (as measured by geometric mean) may correlate with improved performance of a fully-edited HIP CAR-T cell product. Similarly, FIG. 20B shows that higher percentages of CD4+ T cells having active caspase 3 may correlate with improved performance of a fully-edited HIP CAR-T cell product.

Another stage at which a donor cell sample can be assessed is after the sample is enriched for CD4+ T cells and CD8+ T cells (e.g., is an enriched CD4+/CD8+ T cell sample). An enriched CD4+/CD8+ T cell sample comprises CD4+ and/or CD8+ T cells that comprise one or more genetic alterations as provided herein. Additional assessments may be performed, including the characterization of NK cell content in donor CD8+ isolates and effector memory cells (Tem) in donor CD4+ isolates. As shown in FIG. 21A, higher percentages of NK cell content (e.g., CD56+/CD8+ cells) may negatively impact T cell quality and correlate with poor performance of a fully-edited HIP CAR-T cell product. Similarly, as shown in FIG. 21B, higher percentages of Tem cells in CD4+ cell isolates may negatively correlate with performance of a fully-edited HIP CAR-T cell product.

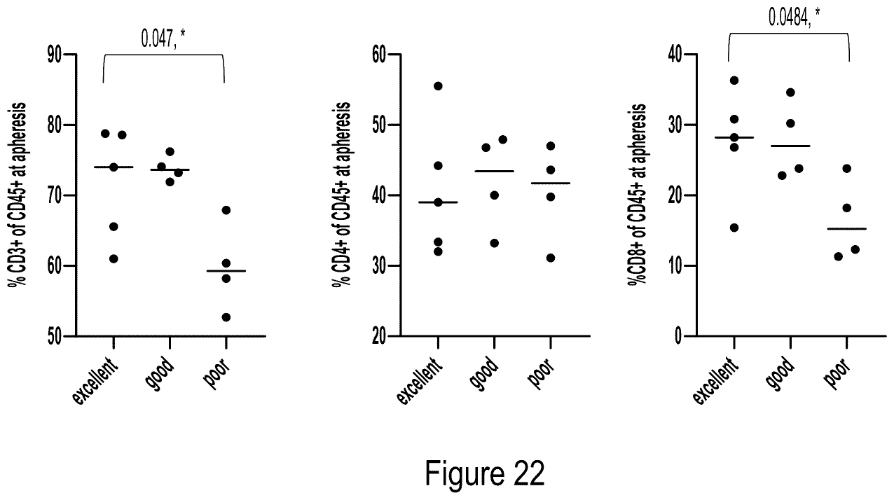

A PBMC panel can also be performed at apheresis to assess T cell population characteristics that may correlate with improved HIP CAR-T cell product performance. For example, a distribution on T cells, monocytes, B cells, NK cells, and granulocytes can be assessed. As shown in FIG. 22, higher total percentages of T cells (CD3+/CD45+) and CD8+ T cells at apheresis may correlate with improved performance of a fully-edited HIP CAR-T cell product.

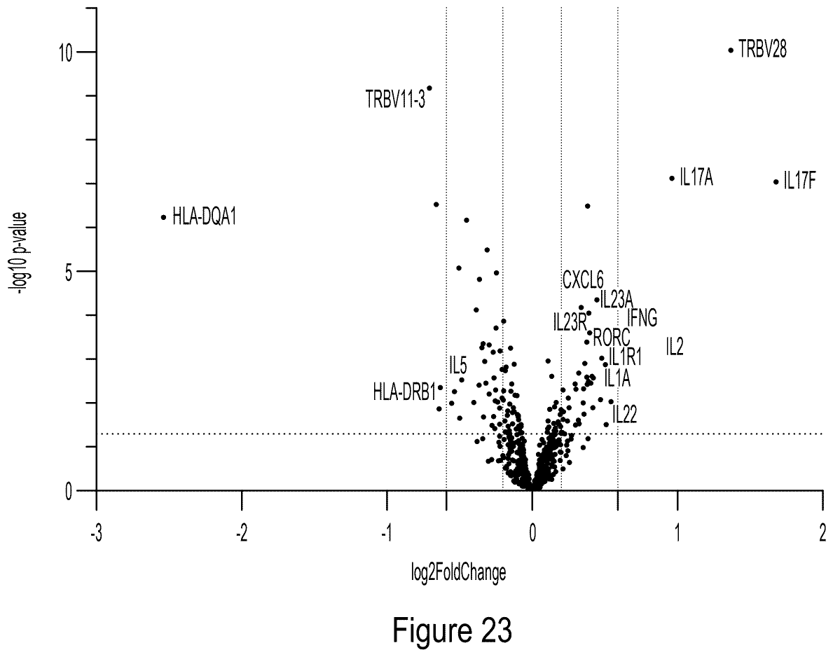

Nanostring nCounter gene expression profiling can additionally be performed to correlate gene expression to improved HIP CAR-T cell product performance. As shown in FIG. 23, an nCounter gene expression panel of 794 genes relating to T cell and CAR T biology was used to assess donor gene profiles. Outperforming donors were observed to have increased expression of T_{c}1/Tₕ1 and T_{c}17/Tₕ17-associated cytokines (e.g., IFNγ, IL17A, IL17F, IL23, and IL22) and reduced expression of T_{c}2/Tₕ2 associated cytokines (e.g., IL5), HLA-DQA1, and HLA-DQB1. Outperforming donors additional showed a mucosal-associated invariant T (MAIT) cell signature including increased expression of TRBV28, RORC, IL23R, and ILR.

Flow cytometry can also be used at any one or more stages including the blood draw prescreen, leukapheresis, PBMC panel to characterize the types of cells and/or ratios of cell types that may correlate with improved HIP CAR-T cell product performance. Flow cytometry can additionally be performed to further characterize T cell subsets, ratios of T cell types, and/or activation of T cell subsets at stage 1. T cell exhaustion markers can also be assessed by flow cytometry at stages 1 and 2. Flow cytometry can also be used to identify T cells that have active caspase-3 as an indication of an activated cell state (e.g., that may not necessary be associated with cell death). Flow cytometry can also be used at stage 2 to measure intracellular cytokines in fully edited HIP CAR T cells at the single-cell level.

TABLE 21
Parameters for Flow Cytometry Analysis and Caspase Assessment

| | | Potential Stage for | Populations and/or Function |
|---|---|---|---|
| Parameter | Description | Assessment | Identified by the Antibody |
| CD3 | T3 receptor (TCR | Blood Draw | T and NK T cells |
| | co-receptor) | Leukapheresis | |
| | | PBMC panel | |
| | | T cell subset panel | |
| | | T cell activation | |
| | | panel | |
| | | Exhaustion panel | |
| | | Caspase-3 panel | |
| CD4 | TCR-co-receptor | Blood Draw | Helper T cells |
| | | Leukapheresis | |
| | | PBMC panel | |
| | | T cell subset panel | |
| | | T cell activation | |
| | | panel | |
| | | Exhaustion Panel | |
| | | Caspase-3 panel | |
| | | Intracellular | |
| | | cytokine Staining of | |
| | | CD19 CAR+ Cells | |
| CD8a | TCR-co-receptor | Blood Draw | Cytotoxic T cells |
| | | Leukapheresis | |
| | | PBMC panel | |
| | | T cell subset panel | |
| | | T cell activation | |
| | | panel | |
| | | Exhaustion Panel | |
| | | Caspase-3 panel | |
| | | Intracellular | |
| | | cytokine Staining of | |
| | | CD19 CAR+ Cells | |
| CD14 | TLR co-receptor | Blood Draw | Monocytes/Macrophages |
| | | Leukapheresis | |
| | | PBMC panel | |
| CD19 | B cell Surface | Blood Draw | B cells |
| | Antigen B4 | Leukapheresis | |
| | | PBMC panel | |
| CD25 | IL-2 Receptor | T cell activation | Activated/Proliferating T cells |
| | (alpha chain) | panel | |
| CD27 | Co-stimulatory | T cell subset panel | CD4/CD8 T cells |
| | receptor | | |
| CD28 | CD80/CD86 | T cell activation | Activated T cells, co-stimulatory |
| | ligand | panel | marker for activation |
| CD39 | NTPDase | Exhaustion panel | Regulates immune response by |
| | | | balancing ATP-ADP |
| CD45 | Protein Tyrosine | Blood Draw | Pan-Hematopoietic Marker |
| | Phosphatase Type | Leukapheresis | |
| | C | PBMC panel | |
| CD45RO | Leukocyte | T cell subset panel | CD45 isoform expressed by memory |
| | Common Antigen | | T cells |
| | RO | | |
| CD56 | NCAM (neural cell | Blood Draw | NK, NK-T cells |
| | adhesion | Leukapheresis | |
| | molecule) | PBMC panel | |
| CD69 | Type II C-lectin | T cell activation | Early Activation marker, may play a |
| | Receptor | panel | role in determining phenotype of T |
| | | | cell |
| CD95 | FasR (pro- | T cell subset panel | Distinguish naive, TSCM, and Teff in |
| | apoptosis receptor) | | CD45RO− ad TCM and TEM in |
| | | | CD45RO+ cells |
| CD223 | Inhibitory co- | Exhaustion Panel | Inhibition of cell proliferation, immune |
| (Lag3) | receptor, binds to | | function, cytokine secretion |
| | MHCII (HLA | | |
| | DP, DQ, DR) | | |
| CD279 | PD-L1 ligand | Exhaustion Panel | Suppresses T cell activation |
| (PD-1) | | | |
| CCR7 | C-C Chemokine | T cell subset panel | Distinguish naive, TSCM, and Teff in |
| | Receptor type 7 | | CD45RO− ad TCM and TEM in |
| | | | CD45RO+ cells |
| Active | Apoptotic cell | Caspase-3 panel | Apoptotic cells |
| Caspase 3 | marker | | |
| HLADR | Human Leukocyte | Blood Draw | Antigen Presenting Cells (APCs) |
| | Antigen DR | Leukapheresis | |
| | | PBMC panel | |
| TIM-3 | Inhibitory receptor | Exhaustion panel | Early Activation marker, may play a |
| | that suppresses | | role in determining phenotype of T cell |
| | cytotoxic T cells | | |
| IL-2 | Cytokine | Intracellular | Aids in cell growth and proliferation |
| | | cytokine Staining of | |
| | | CD19 CAR+ Cells | |
| IL-17a | Cytokine | Intracellular | Has both pro and anti-tumorigenic |
| | | cytokine Staining of | properties. Is associated with an |
| | | CD19 CAR+ Cells | increased risk of neurotoxicity |
| TNFα | Cytokine | Intracellular | of naive T-cells. Can induce apoptosis |
| | | cytokine Staining of | Promotes activation and proliferation |
| | | CD19 CAR+ Cells | of highly activated effector T-cell |
| IFNγ | Cytokine | Intracellular | Acts as cytotoxic CD8 T cell |
| | | cytokine Staining of | differentiation signal |
| | | CD19 CAR+ Cells | essential for induction of cytotoxic T |
| | | | cell. Precursor to proliferation |
| Live/dead | | Blood Draw | Distinguish live from dead cells |
| | | Leukapheresis | |
| | | PBMC panel | |
| | | T cell subset panel | |
| | | T cell activation | |
| | | panel | |
| | | Exhaustion panel | |

### Example 9—Manufacturing Allogeneic CAR-T Cell Therapy

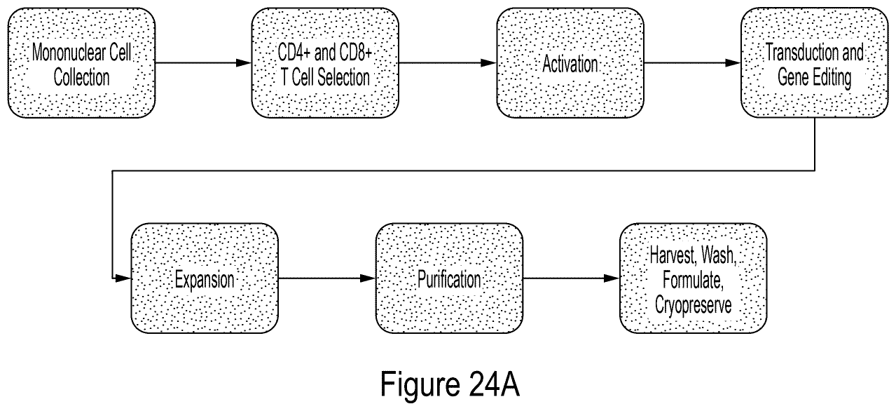

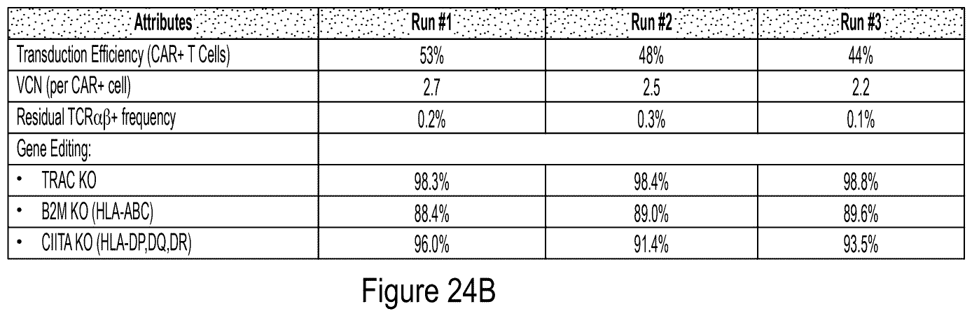

A hypoimmune, CD-19 directed, allogeneic chimeric antigen receptor (CAR) T cell product was generated via an exemplary clinical manufacturing process (FIG. 24a-24b). A post manufacturing analysis shows that the exemplary method is consistent and produces high quality CAR T cell product.

### Methods

### Collecting Mononuclear Cells (MCN) and Isolating Primary Human CD4− and CD8+ T Cells

Enriched, non-mobilized mononuclear cells were obtained from healthy donors via leukapheresis collection. Among other cell types, the collected sample contained CD4+ and CD8+ T cells.

Next, CD4+ and CD8+ Tcells were isolated from the collected sample. Briefly, the cells collected from the donors were centrifuged and washed. Then, the cells were incubated with anti-CD4 and anti-CD8 monoclonal antibodies (conjugated to superparamagnetic iron dextran particles) to label CD4+ and CD8+ cells respectively. Next, the cells were isolated by positive immunomagnetic selection. The positive fraction enriched in CD8+ and CD4+ T cells was washed before proceeding further.

Those of skill in the art will appreciate that CD8+ and CD4+ T cell isolation could be sequential (e.g., CD4+ isolation followed by CD8+ isolation, or CD8+ isolation followed by CD4+ isolation) or simultaneous. Further, those of skill in the art will appreciate that CD8+ isolation and CD4+ isolation could be conducted using any method described herein (e.g., using CD8 and CD4 immunomagnetic selection, CD3/28 based isolation, density-based cell enrichment, FACS sorting, or any combination thereof). In addition, those of skill in the art will appreciate that isolated CD8+ and CD4+ T cells could be formulated for cryopreservation and frozen (combined or separately) prior to proceeding to additional steps.

### Gene Editing and Viral Transduction of Primary Human CD4+ and CD8+ T Cells

CD4+ and CD8+ T cells were incubated and activated. Those of skill in the art will appreciate that CD4+ and CD8+ T cells can be activated using any method descibred herein (e.g., polyclonal activation, CD3 activation (e.g., anti-CD3 antibody), CD28 activation (e.g., anti-CD28 antibody or soluble αCD28), or any combination thereof). Further, those of skill in the art will appreciate that CD4+ and CD8+ T cells can be cultured in media (e.g., T cell expansion media) supplemented with other reagents described herein (e.g., IL2, IL7, IL 15, or a combination thereof).

Next, the activated CD4+ and CD8+ T cells were gene-edited to knock out the B2M, CIITA, and TRAC genes and transduced with virus to yield cells that express CD19CAR and overexpress CD47. Gene-editing was performed using CRISPR/Cas which utilized sgRNAs specific for B2M, CIITA, and TRAC, and CD19CAR expression and CD47 overexpression was performed using viral transduction. Those of skill in the art will appreciate that gene-editing can be performed using any of the methods described herein (e.g., as described in Section II.D, above) and that viral transduction can be performed using any of the methods described herein (e.g., as described in Section II.B, above).

### Purification and Cell Expansion

After gene editing and viral transduction, an immunomagnetic negative selection purification is performed to reduce the levels of TCRαβ+ cells in the final product. Briefly, gene-edited/viral transduced cells were removed from the bioreactor and incubated with a selection reagent. TCRαβ+ cells were removed by positive selection and incubated in T cell expansion media. Those of skill in the art will appreciate that purification of gene-edited virally transduced cells can be performed using any of the methods described herein (e.g., purification using CD3 depletion, TCRa depletion. TCRs depletion, or any combination thereof).

Finally, the gene-edited, virally transduced, purified, expanded allogeneic hypoimmune CD19CAR T cell product was formulated and cryopreserved for later use. Those of skill in the art will appreciate that the formulation of and cryopreservation of gene-edited virally transduced cells can be performed using any of the methods described herein.

### Results

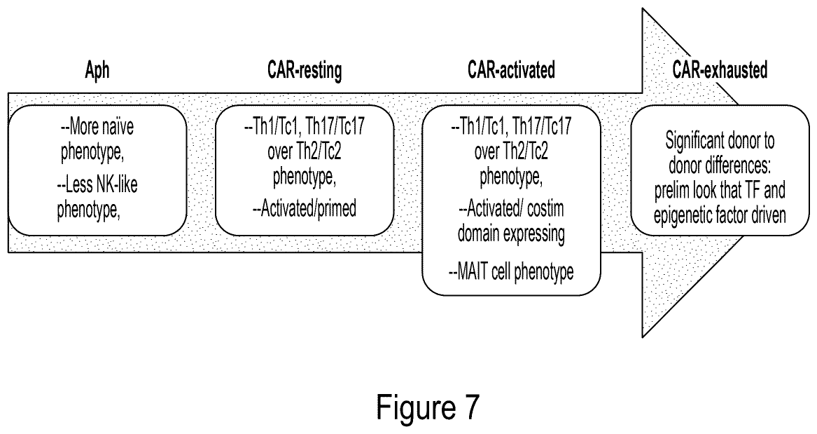

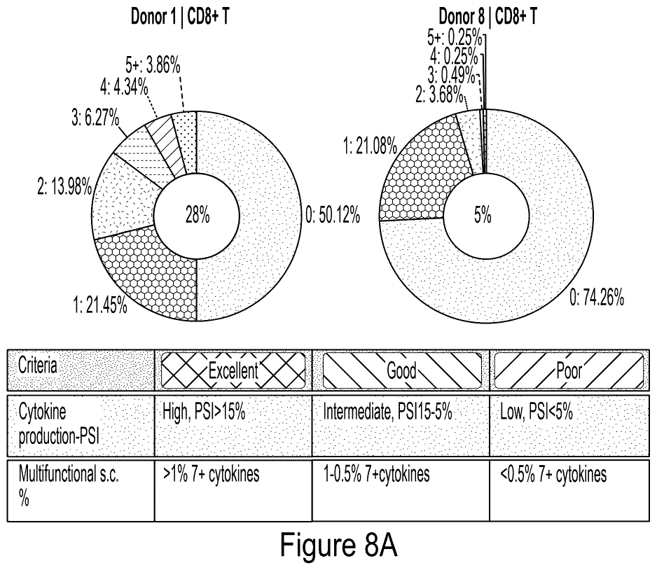

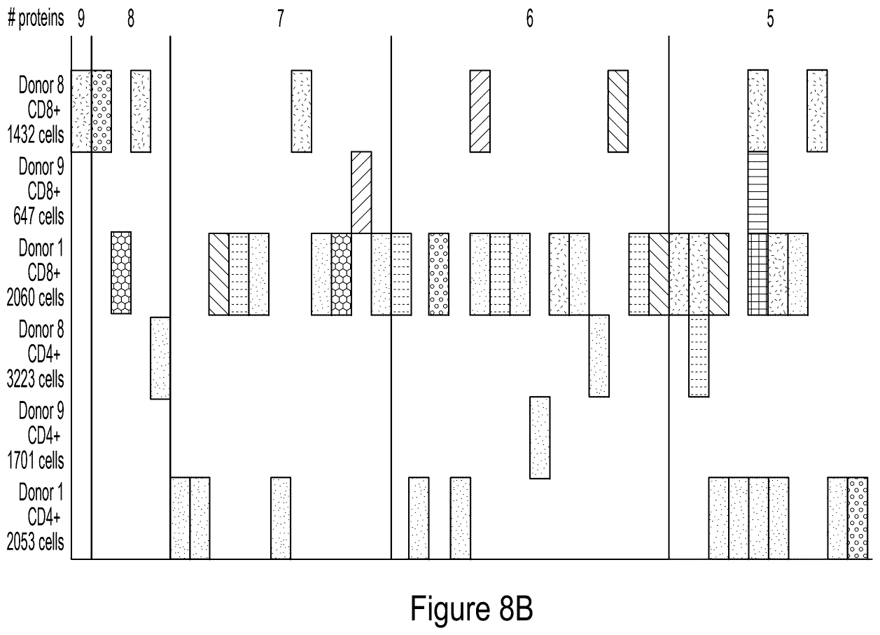

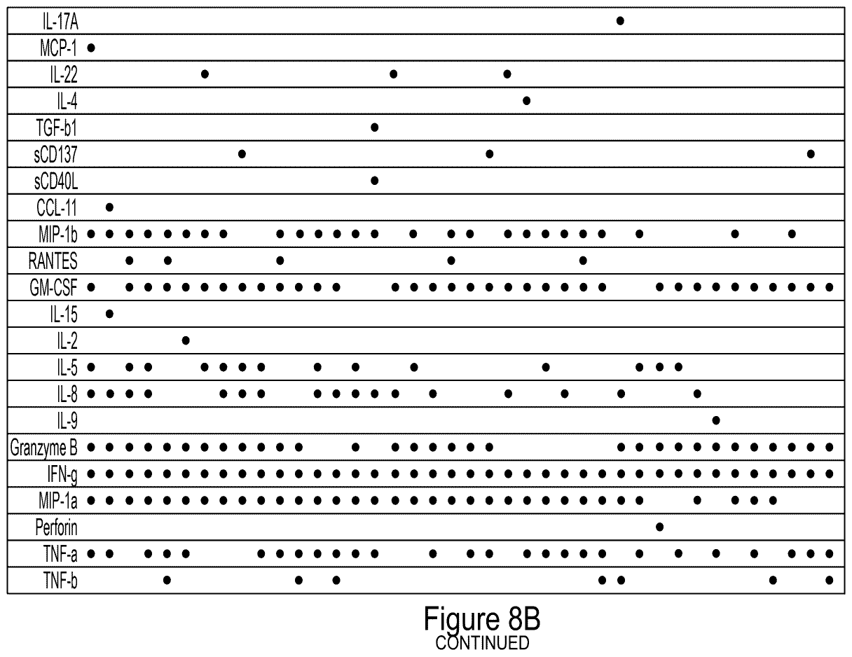

The above manufacturing run was conducted on 3 separate occasions. The cell characteristics (FIG. 7b) demonstrate that the above manufacturing method produces consistent cell product at a commercially viable scale. Briefly, viral transduction efficiency and residual TCRαβ+ cell frequency ranged from 44-53%, and 0.1-0.3%, respectively. The viral copy number (VCN) per CAR+ cell ranged from 2.5 to 2.7 (with the final run yet to be analyzed). Further, the TRAC knock out (TRAC KO) efficiency, B2M knock out (B2M KO) efficiency, and CIITA knock out (CIITA KO) efficiency ranged from 98.3-98.8%, 88.4-86.6%, and 91.4-96.0% respectively. Those of skill in the art will appreciate that assaying for relevant cell/donor characteristics/cell parameters can be conducted at any point during the manufacturing process (e.g., before cell collection, after collection, before gene-editing, after partial gene-editing, after complete gene-editing, before viral transduction, after partial viral transduction, after complete viral transduction, before cell storage (e.g., cryopreservation), after cell storage (e.g., cryopreservation), or any combination thereof).

### Example 10—Evaluation of Donor Quality at Blood Draw

This example describes donor assessments that can be made to predict the functionality of HIP CAR-T cell products derived from those donors.

As previously described in Example 8, T cell quality correlative analysis suggests a significant correlation between lower CD3 cell content and lower CD8 cell content and decreased functionality of CAR T cells derived from those donors. Early timepoint evaluation of donor T cell quality using blood draw material may contribute to reduced costs of manufacturing with higher Stage 1 manufacturing success and improved T cell quality attributes of Stage 2 product.

In this example, whole blood draw samples from donors are analyzed for total CD3, CD4 and CD8 cell content and CD4:CD8 cell ratios using quantification beads and a no-wash method. In brief, 50 μl of whole blood are transferred in triplicate into 5 ml tubes containing a known amount of quantification beads. Cells are stained with anti-CD3, anti-CD4 and anti-CD8 antibodies. After 15 min incubation, red blood cells are lysed with fix-no wash lysing buffer, and samples are evaluated by flow cytometry.

Without wishing to be bound by any particular theory, donors that have CD4:CD8 ratios that are 4:1 or higher (e.g., 4:1, 5:1, 6:1, etc.) are expected to yield insufficient amounts of CD8+ cells at Stage 1 and poorer CAR T functionality. Donors that have CD3 content that is lower than 800 cells/μl, and CD4:CD8 ratios higher than about 2:1 or 3:1, are also expected to yield insufficient cells for manufacturing and poorer T cell quality.

In an alternative method, whole blood draw samples from donors are analyzed to quantify CD4:CD8 relative ratios, the percentage of NK cells, the percentage of HLA-DR+ monocytes, the percentage of iNKT cells, the percentage of CD101+ cell, and/or the percentage of CD39+ cells. In brief, 300 μl of whole blood is transferred in triplicate into 10 ml conical tubes containing antibody panel that allows for PBMC assessment of any one or more of the following cell population and exhaustion markers: CD3. CD4, CD8, MAIT cells, NK cells, Monocytes, iNKT, CD101, and CD39. Cells are stained with anti-CD3, anti-CD4, and anti-CD8 antibodies. After 15 min incubation, red blood cells are lysed and washed. Fixed samples are then evaluated by flow cytometry.

Without wishing to be bound by any particular theory, donors that have have CD4:CD8 ratios that are 4:1 or higher (e.g., 5:1, 6:1, etc.) are expected to yield insufficient amounts of CD8+ at Stage 1 and poorer CAR T functionality. Unusually high NK or monocyte percentages may also correlate with poorer functionality. Cells having low CD39 expression levels and/or high CD101 expression levels are also expected to have poorer functionality.

In yet another method, whole blood draw samples from donors are analyzed to assess basal cytokine status of a donor at draw (definitive). In brief, human adaptive Codeplex Secretome (Isoplexis) platform is used to quantify basal cytokine in 25 μl of a donor serum sample. The assay measures serum levels of one or more of: GM-CSF, Granzyme B, IFN-γ, IL-2, IL-4, IL-5, IL-6, IL-7, IL-8, IL-9, IL-10, IL-13, IL-15, IL-17A, IP-10, MCP-1, MIP-1α, MIP-1β, Perforin, sCD137, TGF-β1, TNF-α, and TNF-β.

As previously described in Example 8, donor Th1 and Th17 population skewing may be correlated with improved HIP CAR T cell functional performance. Without wishing to be bound by any particular theory, CAR T cells derived from donors with higher Th1 and Th17 skewing (as suggested by higher relative IFN-γ, IL-17A, and IL-9 cytokine levels) are expected to be functionally superior to CAR T cells derived from donors with higher Th2 skewing (as suggested by higher relative IL-4 and IL-5 cytokine levels).

Whole blood draw samples may also be used to determine the total percentages and relative ratios of PBMC components for assessing potential correlations with HIP CAR T cell performance. In brief, 5,000 PBMCs are isolated from donor whole blood draw samples and activated with phorbol 12-myristate 13-acetate (PMA)_for 18 hrs. Supernatants are quantified for maximum levels of one or more of the following cytokines: GM-CSF, Granzyme B, IFN-γ, IL-2, IL-4, IL-5, IL-6, IL-7, IL-8, IL-9, IL-10, IL-13, IL-15, IL-17A, IP-10, MCP-1, MIP-1α, MIP-1β, Perforin, sCD137, TGF-β1, TNF-α, and TNF-β.

As previously described in Example 8, donor Th1 and Th17 population skewing may be correlated with improved HIP CAR T cell functional performance. Without wishing to be bound by any particular theory, CAR T cells derived from donors with PBMCs having higher Th1 and Th17 skewing (as suggested by higher relative IFN-γ, IL-17A, and IL-9 levels) are expected to be functionally superior to CAR T cells derived from donors with PBMCs having higher Th2 skewing (as suggested by higher relative IL-4 and IL-5 levels).

Whole blood draw samples may also be used to determine maximal production levels of cytokines in donor CD3+ T cell populations. In brief, 1,000 live CD3+ T cells are isolated form donor whole blood draw samples, and activated with anti-CD3/CD28 antibody for 18 hrs. Supernatant are quantified for maximum levels of one or more of the following cytokines: GM-CSF, Granzyme B, IFN-γ, IL-2, IL-4, IL-5, IL-6, IL-7, IL-8, IL-9, IL-10, IL-13, IL-15, IL-17A, IP-10, MCP-1, MIP-1α, MIP-1β, Perforin, sCD137, TGF-β1, TNF-α, and TNF-β.

As previously described in Example 8, donor Th1 and Th17 population skewing may be correlated with improved HIP CAR T cell functional performance. Without wishing to be bound by any particular theory, CAR T cells derived from donors with higher CD3+ cell Th1 and Th17 skewing (as suggested by higher relative IFN-γ, IL-17A, and IL-9 levels) are expected to be functionally superior to CAR T cells derived from donors with higher CD3+ cell Th2 skewing (as suggested by higher relative IL-4 and IL-5 levels).

### Certain Embodiments

Select embodiments of the disclosed technologies are further described by reference to the following numbered embodiments. It will be understood that the numbered embodiments are intended to be read in combination with the further information provided in the Summary, the Detailed Description, and the Examples.

### First Module of Certain Embodiments

Embodiment 1. A method of making a hypoimmunogenic MAIT cell comprising introducing into a MAIT cell:
   (i) one or more genetic alterations that reduce cell surface expression of a functional Major Histocompatibility Antigen Class I (HLA-I) complex as compared to a reference cell, and/or one or more genetic alterations that reduce cell surface expression of a functional Major Histocompatibility Antigen Class II (HLA-II) complex as compared to a reference cell, and
   (ii) one or more genetic alterations that increase cell surface expression of one or more tolerogenic factors as compared to a reference cell, optionally wherein the one or more tolerogenic factors comprise A20/TNFAIP3, C1-Inhibitor, CCL21, CCL22, CD16, CD16 Fc receptor, CD24, CD27, CD35, CD39, CD46, CD47 CD52, CD55, CD59, CD200, CR1, CTLA4-Ig, DUX4, FasL, H2-M3, HLA-C, HLA-E, HLA-E heavy chain, HLA-F, HLA-G. IDO1, IL-10, IL15-RF, IL-35, MANF, Mfge8, PD-L1, Serpinb9, or any combination thereof.
Embodiment 2. The method according to embodiment 1, wherein the method comprises introducing one or more genetic alterations into a MAIT cell that reduce expression of:
   (i) B2M,
   (ii) CIITA,
   (iii) TCR (e.g., TRAC and/or TRB), or
   (iv) any combination thereof,
as compared to a reference cell.
Embodiment 3. The method according to embodiment 1 or 2, wherein the method comprises introducing one or more genetic alterations into a MAIT cell that knock-out expression of:
   (i) B2M,
   (ii) CIITA.
   (iii) TCR (e.g., TRAC and/or TRB), or
   (iv) any combination thereof.
Embodiment 4. The method according to embodiment 1, wherein the method comprises introducing one or more genetic alterations into a MAIT cell that reduce expression of B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, or any combination thereof, as compared to a reference cell.
Embodiment 5. The method according to any one of the preceding embodiments, wherein the method comprises introducing one or more genetic alterations into a MAIT cell that reduce cell surface expression of a functional HLA-I complex,
   wherein the one or more genetic alterations that reduce cell surface expression of a functional HLA-I complex comprise the one or more genetic alterations that reduce expression of HLA-A, HLA-B, HLA-C, or any combination thereof, as compared to a reference cell.
Embodiment 6. The method according to any one of the preceding embodiments, wherein the method comprises introducing one or more genetic alterations into a MAIT cell that reduce cell surface expression of a functional HLA-II complex,
   wherein the one or more genetic alterations that reduce cell surface expression of a functional HLA-II complex comprise the one or more genetic alterations that reduce expression of HLA-DP, HLA-DQ, HLA-DR, HLA-DM, HLA-DO, or any combination thereof, as compared to a reference cell.
Embodiment 7. The method according to any one of the preceding embodiments, wherein the method comprises introducing one or more genetic alterations into a MAIT cell that reduce expression of RFX5, RFXANK, RFXAP, or any combination thereof, as compared to a reference cell.
Embodiment 8. The method according to any one of embodiments 1-6, wherein the method comprises introducing one or more genetic alterations into a MAIT cell that reduce expression of NFY-A, NFY-B, NFY-C, or any combination thereof, as compared to a reference cell.
Embodiment 9. The method according to any one of embodiments 1-6, wherein the method comprises introducing one or more genetic alterations into a MAIT cell that knock-out expression of B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, or any combination thereof.
Embodiment 10. The method according to any one of the preceding embodiments, wherein the method comprises introducing one or more genetic alterations into a MAIT cell that knock-out cell surface expression of a functional HLA-I complex,
   wherein the one or more genetic alterations that knock-out cell surface expression of a functional HLA-I complex comprise the one or more genetic alterations that reduce expression of HLA-A, HLA-B, HLA-C, or any combination thereof, as compared to a reference cell.
Embodiment 11. The method according to any one of the preceding embodiments, wherein the method comprises introducing one or more genetic alterations into a MAIT cell that knock-out cell surface expression of a functional HLA-II complex,
   wherein the one or more genetic alterations that knock-out cell surface expression of a functional HLA-II complex comprise the one or more genetic alterations that reduce expression of HLA-DP, HLA-DQ, HLA-DR, HLA-DM, HLA-DO, or any combination thereof, as compared to a reference cell.
Embodiment 12. The method according to embodiment 10 or 11, wherein the method comprises introducing one or more genetic alterations into a MAIT cell that knock-out expression of RFX5, RFXANK, RFXAP, or any combination thereof.
Embodiment 13. The method according to embodiment 10 or 11, wherein the method comprises introducing one or more genetic alterations into a MAIT cell that knock-out expression of NFY-A. NFY-B, NFY-C, or any combination thereof.
Embodiment 14. The method according to any one of the preceding embodiments, wherein the method comprises introducing one or more genetic alterations into a MAIT cell that reduce expression of ABO, CADM1, CD58, CD38. CD142, CD155, CEACAM1, FUT1, ICAM1, IRF1, MIC-A, MIC-B, NLGN4Y, PCDH11 Y, PD-1, a protein that is involved in oxidative or ER stress, RHD, TRAC, TRB, or any combination thereof, as compared to a reference cell.
   Embodiment 15. The method according to embodiment 14, wherein the method comprises introducing one or more genetic alterations into a MAIT cell that reduce expression of TRB, optionally wherein TRB comprises TRBC1. TRBC2, or TRBC1 and TRBC2.
Embodiment 16. The method according to any one of the preceding embodiments, wherein the method comprises introducing one or more genetic alterations into a MAIT cell that reduce expression of one or more Y chromosome proteins, as compared to a reference cell,
   optionally wherein the one or more Y chromosome proteins comprise Protocadherin-11 Y-linked (PCDH11 Y) and/or Neuroligin-4 Y-linked (NLGN4Y).
Embodiment 17. The method according to any one of the preceding embodiments, wherein the method comprises introducing one or more genetic alterations into a MAIT cell that reduce expression of one or more NK cell ligands, as compared to a reference cell,
   optionally wherein the one or more NK cell ligands comprise MIC-A and/or MIC-B.
Embodiment 18. The method according to any one of embodiments 1-14, wherein the method comprises introducing one or more genetic alterations into a MAIT cell that reduce expression of one or more proteins involved in oxidative or ER stress, as compared to a reference cell,
   optionally wherein the one or more proteins involved in oxidative or ER stress comprise thioredoxin-interacting protein (TXNIP), PKR-like ER kinase (PERK), inositol-requiring enzyme 1α (IRE1α), and/or DJ-1 (PARK7).
Embodiment 19. The method according to any one of the preceding embodiments, wherein the method comprises introducing one or more genetic alterations into a MAIT cell that reduce expression of one or more additional molecules comprise one or more blood antigen proteins, as compared to a reference cell,
   optionally wherein the one or more blood antigen proteins comprise ABO, FUT1 and/or RHD.
Embodiment 20. The method according to any one of the preceding embodiments, wherein the method comprises introducing one or more genetic alterations into a MAIT cell that:
   (i) reduce expression of MHC class I and/or MHC class II molecules,
   (ii) increase expression of CD47, and
   (iii) increase expression of CCL22,
   as compared to a reference cell.
Embodiment 21. The method according to any one of embodiments 1-19, wherein the method comprises introducing one or more genetic alterations into a MAIT cell that:
   (i) reduce expression of MHC class I and/or MHC class II molecules,
   (ii) increase expression of CD47, and
   (iii) increase expression of CD39,
   as compared to a reference cell.
Embodiment 22. The method according to any one of embodiments 1-19, wherein the method comprises introducing one or more genetic alterations into a MAIT cell that:
   (i) reduce expression of MHC class I and/or MHC class II molecules,
   (ii) increase expression of CD47, and
   (iii) increase expression of CD46 and CD59,
   as compared to a reference cell.
Embodiment 23. The method according to any one of embodiments 1-19, wherein the method comprises introducing one or more genetic alterations into a MAIT cell that:
   (i) reduce expression of MHC class I and/or MHC class II molecules,
   (ii) increase expression of CD47, and
   (iii) increase expression of PD-L1,
   as compared to a reference cell.
Embodiment 24. The method according to any one of embodiments 1-19, wherein the method comprises introducing one or more genetic alterations into a MAIT cell that:
   (i) reduce expression of MHC class I and/or MHC class II molecules,
   (ii) increase expression of CD47; and
   (iii) increase expression of HLA-G and PD-L1,
   as compared to a reference cell.
Embodiment 25. The method according to any one of embodiments 1-19, wherein the method comprises introducing one or more genetic alterations into a MAIT cell that:
   (i) reduce expression of MHC class I and/or MHC class II molecules,
   (ii) increase expression of CD47; and
   (iii) reduced expression of CD142 (TF),
   as compared to a reference cell.
Embodiment 26. The method according to any one of embodiments 1-19, wherein the method comprises introducing one or more genetic alterations into a MAIT cell that:
   (i) reduce expression of MHC class I and/or MHC class II molecules,
   (ii) increase expression of CD47; and
   (iii) reduced expression of MIC-A and/or MIC-B,
   as compared to a reference cell.
Embodiment 27. The method according to any one of embodiments 1-19, wherein the method comprises introducing one or more genetic alterations into a MAIT cell that:
   (i) reduce expression of MHC class I and/or MHC class II molecules, and
   (ii) increase expression of CD24,
   as compared to a reference cell.
Embodiment 28. The method according to any one of embodiments 1-19, wherein the method comprises introducing one or more genetic alterations into a MAIT cell that:
   (i) reduce expression of MHC class I and/or MHC class II molecules, and
   (ii) increase expression of CD200,
   as compared to a reference cell.
Embodiment 29. The method according to any one of embodiments 1-19, wherein the method comprises introducing one or more genetic alterations into a MAIT cell that:
   (i) reduce expression of MHC class I and/or MHC class II molecules, and
   (ii) increase expression of CD52,
   as compared to a reference cell.
Embodiment 30. The method according to any one of embodiments 1-19, wherein the method comprises introducing one or more genetic alterations into a MAIT cell that:
   (i) reduce expression of MHC class I and/or MHC class II molecules; and
   (ii) increase expression of DUX4,
   as compared to a reference cell.
Embodiment 31. The method according to any one of embodiments 1-19, wherein the method comprises introducing one or more genetic alterations into a MAIT cell that:
   (i) reduce expression of MHC class I and/or MHC class II molecules, and
   (ii) increase expression of IDO1,
   as compared to a reference cell.
Embodiment 32. The method according to any one of embodiments 1-19, wherein the method comprises introducing one or more genetic alterations into a MAIT cell that:
   (i) reduce expression of MHC class I and/or MHC class II molecules, and
   (ii) increase expression of IL-35,
   as compared to a reference cell.
Embodiment 33. The method according to any one of embodiments 1-19, wherein the method comprises introducing one or more genetic alterations into a MAIT cell that:
   (i) reduce expression of MHC class I and/or MHC class II molecules, and
   (ii) increase expression of PD-L1,
   as compared to a reference cell.
Embodiment 34. The method according to any one of embodiments 1-19, wherein the method comprises introducing one or more genetic alterations into a MAIT cell that:
   (i) reduce expression of MHC class I and/or MHC class II molecules, and
   (ii) increase expression of HLA-E,
   as compared to a reference cell.
Embodiment 35. The method according to any one of embodiments 1-19, wherein the method comprises introducing one or more genetic alterations into a MAIT cell that:
   (i) reduce expression of MHC class I and/or MHC class II molecules, and
   (ii) increase expression of HLA-G,
   as compared to a reference cell.
Embodiment 36. The method according to any one of embodiments 1-19, wherein the method comprises introducing one or more genetic alterations into a MAIT cell that:
   (i) reduce expression of MHC class I and/or MHC class II molecules,
   (ii) reduce expression of CD155, and
   (iii) increase expression of HLA-E,
   as compared to a reference cell.
Embodiment 37. The method according to any one of embodiments 1-19, wherein the method comprises introducing one or more genetic alterations into a MAIT cell that:
   (i) reduce expression of MHC class I molecules,
   (ii) reduce expression of RFXANK,
   (iii) increase expression of HLA-E,
   as compared to a reference cell.
Embodiment 38. The method according to any one of embodiments 1-19, wherein the method comprises introducing one or more genetic alterations into a MAIT cell that:
   (i) reduce expression of MHC class I and/or MHC class II molecules,
   (ii) reduce expression of MIC-A and/or MIC-B,
   (iii) increase expression of one or more of CD47, CD24 and PD-L1, and
   (iv) increase expression of CD46, CD55, CD59 and CR1,
   as compared to a reference cell.
Embodiment 39. The method according to any one of embodiments 1-19, wherein the method comprises introducing one or more genetic alterations into a MAIT cell that:
   (i) reduce expression of MHC class I molecules,
   (ii) reduce expression of MIC-A and/or MIC-B,
   (iii) reduce expression of TXNIP, and
   (iv) increase expression of PD-L1 and HLA-E,
   as compared to a reference cell.
Embodiment 40. The method according to any one of the preceding embodiments, wherein the method comprises introducing one or more transgenes into the MAIT cell.
Embodiment 41. The method according to embodiment 40, wherein the one or more transgenes encode one or more regulatory elements.
Embodiment 42. The method according to embodiment 41, wherein the one or more transgenes are operably linked to the one or more regulatory elements.
Embodiment 43. The method according to embodiment 41 or 42, wherein the one or more regulatory elements comprise one or more promoters, enhancers, introns, terminators, translation initiation signals, polyadenylation signals, replication elements, RNA processing and export elements, transposons, transposases, insulators, internal ribosome entry sites (IRES), 5′UTRs, 3′UTRs, mRNA 3′ end processing sequences, boundary elements, locus control regions (LCR), matrix attachment regions (MAR), recombination or cassette exchange sequences, linker sequences, secretion signals, resistance markers, anchoring peptides, localization signals, fusion tags, affinity tags, chaperonins, proteases, or any combination thereof.
Embodiment 44. The method according to any one of embodiments 40-43, wherein the one or more transgenes are part of one or more vectors.
Embodiment 45. The method according to embodiment 44, wherein at least one of the one or more vectors is a multicistronic vector.
Embodiment 46. The method according to embodiment 45, wherein the multicistronic vector encodes at least one of the one or more tolerogenic factors.
Embodiment 47. The method according to any one of embodiments 40-46, wherein the one or more transgenes are separated by one or more linker sequences.
Embodiment 48. The method according to embodiment 47, wherein the one or more linker sequences comprise an IRES sequence or a cleavable peptide sequence.
Embodiment 49. The method according to embodiment 48, wherein the cleavable peptide sequence comprises a self-cleavable peptide, optionally a 2A peptide.
Embodiment 50. The method according to embodiment 49, wherein the 2A peptide is selected from the group consisting of a F2A sequence, an E2A sequence, a P2A sequence, and a T2A sequence.
Embodiment 51. The method according to any one of embodiment 48-50, wherein the cleavable peptide sequence comprises a protease cleavable sequence or a chemically cleavable sequence.
Embodiment 52. The method according to any one of the preceding embodiments, wherein the method comprises introducing a chimeric antigen receptor (CAR) into a MAIT cell.
Embodiment 53. The method according to embodiment 52, wherein the CAR comprises a signal peptide, an extracellular binding domain specific to CD19, a hinge domain, a transmembrane domain, an intracellular costimulatory domain, and/or an intracellular signaling domain.
Embodiment 54. The method according to embodiment 53, wherein the CAR comprises a CD5-specific CAR, a CD19-specific CAR, a CD20-specific CAR, a CD22-specific CAR, a CD23-specific CAR, a CD30-specific CAR, a CD33-specific CAR, CD38-specific CAR, a CD70-specific CAR, a CD123-specific CAR, a CD138-specific CAR, a Kappa, Lambda, B cell maturation agent (BCMA)-specific CAR, a G-protein coupled receptor family C group 5 member D (GPRC5D)-specific CAR, a CD123-specific CAR, a LeY-specific CAR, a NKG2D ligand-specific CAR, a WT1-specific CAR, a GD2-specific CAR, a HER2-specific CAR, a EGFR-specific CAR, a EGFRvIII-specific CAR, a B7H3-specific CAR, a PSMA-specific CAR, a PSCA-specific CAR, a CAIX-specific CAR, a CD171-specific CAR, a CEA-specific CAR, a CSPG4-specific CAR, a EPHA2-specific CAR, a FAP-specific CAR, a FRa-specific CAR, a IL-13Rα-specific CAR, a Mesothelin-specific CAR, a MUC1-specific CAR, a MUC16-specific CAR, a ROR1-specific CAR, a C-Met-specific CAR, a CD133-specific CAR, a Ep-CAM-specific CAR, a GPC3-specific CAR, a HPV16-E6-specific CAR, a IL13Ra2-specific CAR, a MAGEA3-specific CAR, a MAGEA4-specific CAR, a MART1-specific CAR, a NY-ESO-1-specific CAR, a VEGFR2-specific CAR, a α-Folate receptor-specific CAR, a CD24-specific CAR, a CD44v7/8-specific CAR, a EGP-2-specific CAR, a EGP-40-specific CAR, a erb-B2-specific CAR, a erb-B 2.3.4-specific CAR, a FBP-specific CAR, a Fetal acethylcholine e receptor-specific CAR, a GD2-specific CAR, a GD3-specific CAR, a HMW-MAA-specific CAR, a IL-11Rα-specific CAR, a KDR-specific CAR, a Lewis Y-specific CAR, a L1-cell adhesion molecule-specific CAR, a MAGE-A1-specific CAR, a Oncofetal antigen (h5T4)-specific CAR, a TAG-72-specific CAR, or a CD19/CD22-bispecific CAR.
Embodiment 55. The method according to any one of embodiments 52-54, wherein the CAR comprises a CD19-specific CAR, a CD20-specific CAR, a CD22-specific CAR, a CD38-specific CAR, a CD123-specific CAR, a CD138-specific CAR, a BCMA-specific CAR, or a CD19/CD22-bispecific CAR
Embodiment 56. The method according to embodiment 55, wherein the CAR is specific for CD19.
Embodiment 57. The method according to embodiment 55, wherein the CAR is specific for CD22.
Embodiment 58. The method according to 55, wherein the CAR is a CD19/CD22-bispecific CAR.
Embodiment 59. The method according to any one of the preceding embodiments, wherein the method comprises introducing one or more safety switches into a MAIT cell.
Embodiment 60. The method according to embodiment 59, wherein the one or more safety switches are capable of controlled killing of the cell or the population of cells.
Embodiment 61. The method according to embodiment 59 or 60, wherein the one or more safety switches induce controlled cell death in the presence of a drug or prodrug, or upon activation by a selective exogenous compound.
Embodiment 62. The method according to any one of embodiments 59-61, wherein the one or more safety switches comprise is an inducible protein capable of inducing apoptosis of the cell or the population of cells.
Embodiment 63. The method according to embodiment 62, wherein the inducible protein capable of inducing apoptosis of the cell or the population of cells is a caspase protein.
Embodiment 64. The method according to embodiment 63, wherein the caspase protein is caspase 9.
Embodiment 65. The method according to anyone of embodiments 59-64, wherein the one or more safety switches comprise one or more suicide genes.
Embodiment 66. The method according to embodiment 65, wherein the one or more suicide genes are selected from the group consisting of cytosine deaminase (CyD), herpesvirus thymidine kinase (HSV-Tk), an inducible caspase 9 (iCaspase9), and rapamycin-activated caspase 9 (rapaCasp9).
Embodiment 67. The method according to any one of embodiments 59-66, wherein the one or more safety switch is an uncloaking system wherein upon activation, cells downregulate expression of immunosuppressive factors and/or upregulate expression of immune signaling molecules thereby reducing the hypoimmunogenicity of the cell.
Embodiment 68. The method according to any one of embodiments 59-67, wherein introducing one or more transgenes, a CAR, and/or one or more safety switches comprises integrating at least one of the one or more transgenes, a CAR, and/or one or more safety switches into the genome of the MAIT cell.
Embodiment 69. The method according to embodiment 68, wherein integration is by non-targeted insertion into the genome of the MAIT cell.
Embodiment 70. The method according to embodiment 68, wherein integration is by targeted insertion into a target genomic locus of the MAIT cell.
Embodiment 71. The method according to embodiment 70, wherein targeted insertion utilizes homology-directed repair.
Embodiment 72. The method according to embodiment 70 or 71, wherein the target genomic locus is an albumin gene locus, an ABO gene locus, a B2M gene locus, a CIITA gene locus, a CCR5 gene locus, a CD142 gene locus, a CLYBL gene locus, a CXCR4 gene locus, an F3 gene locus, a FUT1 gene locus, an HMGB1 gene locus, a KDM5D gene locus, an LRP1 gene locus, a MIC-A gene locus, a MIC-B gene locus, a PPPIR12C (also known as AAVS1) gene locus, an RHD gene locus, a ROSA26 gene locus, a safe harbor gene locus, a SHS231 locus, a TAP1 gene locus, a TRAC gene locus, or a TRBC gene locus of the MAIT cell.
Embodiment 73. The method according to embodiment 68, wherein introducing one or more transgenes, a CAR, and/or one or more safety switches is performed using a genome editing complex.
Embodiment 74. The method according to embodiment 73, wherein the genome editing complex comprises a genome targeting entity and a genome modifying entity.
Embodiment 75. The method according to embodiment 74, comprising localizes the genome editing complex to the target locus using the genome targeting entity, optionally wherein the genome targeting entity is a nucleic acid-guided targeting entity.
Embodiment 76. The method according to embodiment 74 or 75, wherein the genome targeting entity comprises a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZF) nucleic acid binding entity, a transcription activator-like effector (TALE) nucleic acid binding entity, a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, or a functional portion thereof.
Embodiment 77. The method according to any one of embodiments 74-76, wherein the genome targeting entity comprises Cas1, Cas2, Cas3, Cas4. Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, dCas13b, or a functional portion thereof.
Embodiment 78. The method according to any one of embodiments 74-77, wherein the genome modifying entity cleaves, deaminates, nicks, polymerizes, interrogates, integrates, cuts, unwinds, breaks, alters, methylates, demethylates, or otherwise destabilizes the target locus.
Embodiment 79. The method according to any one of embodiments 74-78, wherein the genome modifying entity comprises a recombinase, integrase, transposase, endonuclease, exonuclease, nickase, helicase, DNA polymerase. RNA polymerase, reverse transcriptase, deaminase, flippase, methylase, demethylase, acetylase, a nucleic acid modifying protein, an RNA modifying protein, a DNA modifying protein, an Argonaute protein, an epigenetic modifying protein, a histone modifying protein, or a functional portion thereof.
Embodiment 80. The method according to any one of embodiments 74-79, wherein the genome modifying entity comprises a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, base editing, prime editing, a Programmable Addition via Site-specific Targeting Elements (PASTE), or a functional portion thereof.
Embodiment 81. The method according to any one of embodiment 74-80, wherein the genome modifying entity comprises Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX). Cas12f (C2c10), Cas12g. Cas12h, Cas12i. Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, FokI, dCas (D10A), dCas (H840A), dCas13a, dCas13b, a base editor, a prime editor (e.g., a target-primed reverse transcription (TPRT) editor), APOBEC1, cytidine deaminase, adenosine deaminase, uracil glycosylase inhibitor (UGI), adenine base editors (ABE), cytosine base editors (CBE), reverse transcriptase, serine integrase, recombinase, transposase, polymerase, adenine-to-thymine or “ATBE” (or thymine-to-adenine or “TABE”) transversion base editor, ten-eleven translocation methylcytosine dioxygenases (TETs), TET1, TET3, TET1CD, histone acetyltransferase p300, histone methyltransferase SMYD3, histone methyltransferase PRDM9, H3K79 methyltransferase DOT1L, transcriptional repressor, or a functional portion thereof.
Embodiment 82. The method according to any one of embodiments 74-81, wherein the genome targeting entity and the genome modifying entity are different domains of a single polypeptide.
Embodiment 83. The method according to any one of embodiments 74-82, wherein the genome editing entity and genome modifying entity are two different polypeptides that are operably linked together.
Embodiment 84. The method according to any one of embodiments 74-82, wherein the genome editing entity and genome modifying entity are two different polypeptides that are not linked together.
Embodiment 85. The method according to any one of embodiments 74-84, wherein the genome editing complex comprises a guide nucleic acid having a targeting domain that is complementary to at least one target locus, optionally wherein the guide nucleic acid is a guide RNA (gRNA).
Embodiment 86. A mucosal-associated invariant T (MAIT) cell comprising:
   (i) one or more genetic alterations that reduce cell surface expression of a functional Major Histocompatibility Antigen Class I (HLA-I) complex as compared to a reference cell, and/or one or more genetic alterations that reduce cell surface expression of a functional Major Histocompatibility Antigen Class II (HLA-II) complex as compared to a reference cell, and
   (ii) one or more genetic alterations that increase cell surface expression of one or more tolerogenic factors as compared to a reference cell, optionally wherein the one or more tolerogenic factors comprise CD47.
Embodiment 87. The MAIT cell or method according to embodiment 86, wherein the MAIT cell is hypoimmunogenic as compared to a reference cell.
Embodiment 88. The MAIT cell or method according to embodiment 86 or 87, wherein the MAIT cell comprises one or more genetic alterations that reduce expression of:
   (i) B2M,
   (ii) CIITA,
   (iii) TCR (e.g., TRAC and/or TRB), or
   (iv) any combination thereof,
as compared to a reference cell.
Embodiment 89. The MAIT cell or method according to any one of embodiments 86-88, wherein the MAIT cell comprises one or more genetic alterations that knock-out expression of:
   (i) B2M,
   (ii) CIITA,
   (iii) TCR (e.g., TRAC and/or TRB), or
   (iv) any combination thereof.
Embodiment 90. The MAIT cell or method according to any one of embodiments 86-89, wherein the MAIT cell comprises one or more genetic alterations that reduce expression of B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, or any combination thereof, as compared to a reference cell.
Embodiment 91. The MAIT cell or method according to any one of embodiments 86-90, wherein the one or more genetic alterations that reduce cell surface expression of a functional HLA-I complex comprise one or more genetic alterations that reduce expression of HLA-A, HLA-B, HLA-C, or any combination thereof, as compared to a reference cell.
Embodiment 92. The MAIT cell or method according to any one of embodiments 86-91, wherein the one or more genetic alterations that reduce cell surface expression of a functional HLA-II complex comprise one or more genetic alterations that reduce expression of HLA-DP, HLA-DQ, HLA-DR, HLA-DM, HLA-DO, or any combination thereof, as compared to a reference cell.
Embodiment 93. The MAIT cell or method according to any one of embodiments 86-92 wherein the MAIT cell comprises one or more genetic alterations that reduce expression of RFX5, RFXANK, RFXAP, or any combination thereof, as compared to a reference cell.
Embodiment 94. The MAIT cell or method according to any one of embodiments 86-92, wherein the MAIT cell comprises one or more genetic alterations that reduce expression of NFY-A, NFY-B, NFY-C, or any combination thereof, as compared to a reference cell.
Embodiment 95. The MAIT cell or method according to any one of embodiments 86-92, wherein the MAIT cell comprises one or more genetic alterations that knock-out expression of B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, or any combination thereof.
Embodiment 96. The MAIT cell or method according to any one of embodiments 86-92, wherein the MAIT cell comprises one or more genetic alterations that knock-out expression of HLA-A, HLA-B, HLA-C, or any combination thereof.
Embodiment 97. The MAIT cell or method according to embodiment 95 or 96, wherein the MAIT cell comprises one or more genetic alterations that knock-out expression of HLA-DP. HLA-DQ, HLA-DR, HLA-DM, HLA-DO, or any combination thereof.
Embodiment 98. The MAIT cell or method according to embodiment 95 or 96, wherein the MAIT cell comprises one or more genetic alterations that knock-out expression of RFX5, RFXANK, RFXAP, or any combination thereof.
Embodiment 99. The MAIT cell or method according to embodiment 95 or 96, wherein the MAIT cell comprises one or more genetic alterations that knock-out expression of NFY-A, NFY-B, NFY-C, or any combination thereof.
Embodiment 100. The MAIT cell or method according to any one of embodiments 86-99, wherein the one or more tolerogenic factors comprise A20/TNFAIP3, C1-Inhibitor, CCL21, CCL22, CD16, CD16 Fc receptor, CD24, CD27, CD35, CD39, CD46, CD52, CD55, CD59, CD200, CR1, CTLA4-Ig, DUX4, FasL. H2-M3, HLA-C, HLA-E, HLA-E heavy chain, HLA-F, HLA-G, IDO1, IL-10, IL15-RF, IL-35, MANF, Mfge8, PD-L1, Serpinb9, or any combination thereof.
Embodiment 101. The MAIT cell or method according to any one of embodiments 86-100, wherein the one or more tolerogenic factors comprise CD47, CD46, CD59, or any combination thereof, as compared to a reference cell.
Embodiment 102. The MAIT cell or method according to any one of embodiments 86-100, wherein the one or more tolerogenic factors comprise CD39, CCL22, or any combination thereof, as compared to a reference cell.
Embodiment 103. The MAIT cell or method according to any one of embodiments 86-100, wherein the one or more tolerogenic factors comprise HLA-G, PD-L1, or any combination thereof, as compared to a reference cell.
Embodiment 104. The MAIT cell or method according to any one of embodiments 86-100, wherein the one or more tolerogenic factors comprise CD24, PD-L1, or any combination thereof, as compared to a reference cell.
Embodiment 105. The MAIT cell or method according to any one of embodiments 86-100, wherein the one or more tolerogenic factors comprise HLA-E, CD24. PD-L1, or any combination thereof, as compared to a reference cell.
Embodiment 106. The MAIT cell or method according to any one of embodiments 86-100, wherein the one or more tolerogenic factors comprise CD46, CD55, or any combination thereof, as compared to a reference cell.
Embodiment 107. The MAIT cell or method according to any one of embodiments 86-100, wherein the one or more tolerogenic factors comprise CD46, CD55, CD59, or any combination thereof, as compared to a reference cell.
Embodiment 108. The MAIT cell or method according to any one of embodiments 86-100, wherein the one or more tolerogenic factors comprise CD46, CD55, CD59. CR1, or any combination thereof, as compared to a reference cell.
Embodiment 109. The MAIT cell or method according to any one of embodiments 86-100, wherein the one or more tolerogenic factors comprise HLA-E, CD46. CD55, CD59, CR1, or any combination thereof, as compared to a reference cell.
Embodiment 110. The MAIT cell or method according to any one of embodiments 86-100, wherein the one or more tolerogenic factors comprise HLA-E, CD24, CD47, PD-L1, CD46, CD55, CD59, CR1, or any combination thereof, as compared to a reference cell.
Embodiment 111. The MAIT cell or method according to any one of embodiments 86-100, wherein the one or more tolerogenic factors comprise HLA-E, PD-L1, A20NFAIP, or any combination thereof, as compared to a reference cell.
Embodiment 112. The MAIT cell or method according to any one of embodiments 86-100, wherein the one or more tolerogenic factors comprise HLA-E, PD-L1, MANF, or any combination thereof, as compared to a reference cell.
Embodiment 113. The MAIT cell or method according to any one of embodiments 86-100, wherein the one or more tolerogenic factors comprise HLA-E, PD-L1, A20TNFAIP, MANF, or any combination thereof, as compared to a reference cell.
Embodiment 114. The MAIT cell or method according to any one of embodiments 86-113, wherein the MAIT cell comprises one or more genetic alterations that reduce expression of ABO, CADM1, CD58, CD38. CD142, CD155, CEACAM1, FUT1, ICAM1, IRF1, MIC-A, MIC-B, NLGN4Y, PCDH11Y, PD-1, a protein that is involved in oxidative or ER stress, RHD. TRAC, TRB, or any combination thereof, as compared to a reference cell.
Embodiment 115. The MAIT cell or method according to embodiment 114, wherein the MAIT cell comprises one or more genetic alterations that reduce expression of TRB, optionally wherein TRB comprises TRBC1, TRBC2, or TRBC1 and TRBC2.
Embodiment 116. The MAIT cell or method according to any one of embodiments 86-115, wherein the MAIT cell comprises one or more genetic alterations that reduce expression of one or more Y chromosome proteins, as compared to a reference cell,
   optionally wherein the one or more Y chromosome proteins comprise Protocadherin-11 Y-linked (PCDH11 Y) and/or Neuroligin-4 Y-linked (NLGN4Y).
Embodiment 117. The MAIT cell or method according to any one of embodiments 86-116, wherein the MAIT cell comprises one or more genetic alterations that reduce expression of one or more NK cell ligands, as compared to a reference cell,
   optionally wherein the one or more NK cell ligands comprise MIC-A and/or MIC-B.
Embodiment 118. The MAIT cell or method according to any one of embodiments 86-115, wherein the MAIT cell comprises one or more genetic alterations that reduce expression of one or more proteins involved in oxidative or ER stress, as compared to a reference cell, optionally wherein the one or more proteins involved in oxidative or ER stress comprise thioredoxin-interacting protein (TXNIP), PKR-like ER kinase (PERK), inositol-requiring enzyme 1α (IRE1α), and/or DJ-1 (PARK7).
Embodiment 119. The MAIT cell or method according to any one of embodiments 86-118, wherein the MAIT cell comprises one or more genetic alterations that reduce expression of one or more additional molecules comprise one or more blood antigen proteins, as compared to a reference cell,
   optionally wherein the one or more blood antigen proteins comprise ABO, FUT1 and/or RHD.
Embodiment 120. The MAIT cell or method according to any one of embodiments 86-119, wherein the MAIT cell comprises one or more genetic alterations that:
   (i) reduce expression of MHC class I and/or MHC class II molecules,
   (ii) increase expression of CD47, and
   (iii) increase expression of CCL22,
   as compared to a reference cell.
Embodiment 121. The MAIT cell or method according to any one of embodiments 86-119, wherein the MAIT cell comprises one or more genetic alterations that:
   (i) reduce expression of MHC class I and/or MHC class II molecules,
   (ii) increase expression of CD47, and
   (iii) increase expression of CD39,
   as compared to a reference cell.
Embodiment 122. The MAIT cell or method according to any one of embodiments 86-119, wherein the MAIT cell comprises one or more genetic alterations that:
   (i) reduce expression of MHC class I and/or MHC class II molecules,
   (ii) increase expression of CD47, and
   (iii) increase expression of CD46 and CD59,
   as compared to a reference cell.
Embodiment 123. The MAIT cell or method according to any one of embodiments 86-119, wherein the MAIT cell comprises one or more genetic alterations that:
   (i) reduce expression of MHC class I and/or MHC class II molecules,
   (ii) increase expression of CD47, and
   (iii) increase expression of PD-L1,
   as compared to a reference cell.
Embodiment 124. The MAIT cell or method according to any one of embodiments 86-119, wherein the MAIT cell comprises one or more genetic alterations that:
   (i) reduce expression of MHC class I and/or MHC class II molecules,
   (ii) increase expression of CD47; and
   (iii) increase expression of HLA-G and PD-L1,
   as compared to a reference cell.
Embodiment 125. The MAIT cell or method according to any one of embodiments 86-119, wherein the MAIT cell comprises one or more genetic alterations that:
   (i) reduce expression of MHC class I and/or MHC class II molecules,
   (ii) increase expression of CD47; and
   (iii) reduced expression of CD142 (TF),
   as compared to a reference cell.
Embodiment 126. The MAIT cell or method according to any one of embodiments 86-119, wherein the MAIT cell comprises one or more genetic alterations that:
   (i) reduce expression of MHC class I and/or MHC class II molecules,
   (ii) increase expression of CD47; and
   (iii) reduced expression of MIC-A and/or MIC-B,
   as compared to a reference cell.
Embodiment 127. The MAIT cell or method according to any one of embodiments 86-119, wherein the MAIT cell comprises one or more genetic alterations that:
   (i) reduce expression of MHC class I and/or MHC class II molecules, and
   (ii) increase expression of CD24,
   as compared to a reference cell.
Embodiment 128. The MAIT cell or method according to any one of embodiments 86-119, wherein the MAIT cell comprises one or more genetic alterations that:
   (i) reduce expression of MHC class I and/or MHC class II molecules, and
   (ii) increase expression of CD200,
   as compared to a reference cell.
Embodiment 129. The MAIT cell or method according to any one of embodiments 86-119, wherein the MAIT cell comprises one or more genetic alterations that:
   (i) reduce expression of MHC class I and/or MHC class II molecules, and
   (ii) increase expression of CD52,
   as compared to a reference cell.
Embodiment 130. The MAIT cell or method according to any one of embodiments 86-119, wherein the MAIT cell comprises one or more genetic alterations that:
   (i) reduce expression of MHC class I and/or MHC class II molecules; and
   (ii) increase expression of DUX4,
   as compared to a reference cell.
Embodiment 131. The MAIT cell or method according to any one of embodiments 86-119, wherein the MAIT cell comprises one or more genetic alterations that:
   (i) reduce expression of MHC class I and/or MHC class II molecules, and
   (ii) increase expression of IDO1,
   as compared to a reference cell.
Embodiment 132. The MAIT cell or method according to any one of embodiments 86-119, wherein the MAIT cell comprises one or more genetic alterations that:
   (i) reduce expression of MHC class I and/or MHC class II molecules, and
   (ii) increase expression of IL-35,
   as compared to a reference cell.
Embodiment 133. The MAIT cell or method according to any one of embodiments 86-119, wherein the MAIT cell comprises one or more genetic alterations that:
   (i) reduce expression of MHC class I and/or MHC class II molecules, and
   (ii) increase expression of PD-L1,
   as compared to a reference cell.
Embodiment 134. The MAIT cell or method according to any one of embodiments 86-119, wherein the MAIT cell comprises one or more genetic alterations that:
   (i) reduce expression of MHC class I and/or MHC class II molecules, and
   (ii) increase expression of HLA-E,
   as compared to a reference cell.
Embodiment 135. The MAIT cell or method according to any one of embodiments 86-119, wherein the MAIT cell comprises one or more genetic alterations that:
   (i) reduce expression of MHC class I and/or MHC class II molecules, and
   (ii) increase expression of HLA-G,
   as compared to a reference cell.
Embodiment 136. The MAIT cell or method according to any one of embodiments 86-119, wherein the MAIT cell comprises one or more genetic alterations that:
   (i) reduce expression of MHC class I and/or MHC class II molecules,
   (ii) reduce expression of CD155, and
   (iii) increase expression of HLA-E,
   as compared to a reference cell.
Embodiment 137. The MAIT cell or method according to any one of embodiments 86-119, wherein the MAIT cell comprises one or more genetic alterations that:
   (i) reduce expression of MHC class I molecules,
   (ii) reduce expression of RFXANK,
   (iii) increase expression of HLA-E,
   as compared to a reference cell.
Embodiment 138. The MAIT cell or method according to any one of embodiments 86-119, wherein the MAIT cell comprises one or more genetic alterations that:
   (i) reduce expression of MHC class I and/or MHC class II molecules,
   (ii) reduce expression of MIC-A and/or MIC-B,
   (iii) increase expression of one or more of CD47, CD24 and PD-L1, and
   (iv) increase expression of CD46, CD55, CD59 and CR1,
   as compared to a reference cell.
Embodiment 139. The MAIT cell or method according to any one of embodiments 86-119, wherein the MAIT cell comprises one or more genetic alterations that:
   (i) reduce expression of MHC class I molecules,
   (ii) reduce expression of MIC-A and/or MIC-B,
   (iii) reduce expression of TXNIP, and
   (iv) increase expression of PD-L1 and HLA-E,
   as compared to a reference cell.
Embodiment 140. The MAIT cell or method according to any one of embodiments 86-140, wherein the MAIT cell comprises one or more transgenes.
Embodiment 141. The MAIT cell or method according to embodiment 140, wherein the one or more transgenes encode one or more regulatory elements.
Embodiment 142. The MAIT cell or method according to embodiment 141, wherein the one or more transgenes are operably linked to the one or more regulatory elements.
Embodiment 143. The MAIT cell or method according to embodiment 141 or 142, wherein the one or more regulatory elements comprise one or more promoters, enhancers, introns, terminators, translation initiation signals, polyadenylation signals, replication elements, RNA processing and export elements, transposons, transposases, insulators, internal ribosome entry sites (IRES), 5′UTRs, 3′UTRs, mRNA 3′ end processing sequences, boundary elements, locus control regions (LCR), matrix attachment regions (MAR), recombination or cassette exchange sequences, linker sequences, secretion signals, resistance markers, anchoring peptides, localization signals, fusion tags, affinity tags, chaperonins, proteases, or any combination thereof.
Embodiment 144. The MAIT cell or method according to any one of embodiments 140-143, wherein the one or more transgenes are part of one or more vectors.
Embodiment 145. The MAIT cell or method according to embodiment 144, wherein at least one of the one or more vectors is a multicistronic vector.
Embodiment 146. The MAIT cell or method according to embodiment 145, wherein the multicistronic vector encodes at least one of the one or more tolerogenic factors.
Embodiment 147. The MAIT cell or method according to any one of embodiments 140-146, wherein the one or more transgenes are separated by one or more linker sequences.
Embodiment 148. The MAIT cell or method according to embodiment 147, wherein the one or more linker sequences comprise an IRES sequence or a cleavable peptide sequence.
Embodiment 149. The MAIT cell or method according to embodiment 148, wherein the cleavable peptide sequence comprises a self-cleavable peptide, optionally a 2A peptide.
Embodiment 150. The MAIT cell or method according to embodiment 149, wherein the 2A peptide is selected from the group consisting of a F2A sequence, an E2A sequence, a P2A sequence, and a T2A sequence.
Embodiment 151. The MAIT cell or method according to any one of embodiments 148-150, wherein the cleavable peptide sequence comprises a protease cleavable sequence or a chemically cleavable sequence.
Embodiment 152. The MAIT cell or method according to any one of embodiments 86-151, wherein the MAIT cell comprises a chimeric antigen receptor (CAR).
Embodiment 153. The MAIT cell or method according to embodiments 152, wherein the CAR comprises a signal peptide, an extracellular binding domain specific to CD19, a hinge domain, a transmembrane domain, an intracellular costimulatory domain, and/or an intracellular signaling domain.
Embodiment 154. The MAIT cell or method according to embodiment 153, wherein the CAR comprises a CD5-specific CAR, a CD19-specific CAR, a CD20-specific CAR, a CD22-specific CAR, a CD23-specific CAR, a CD30-specific CAR, a CD33-specific CAR, CD38-specific CAR, a CD70-specific CAR, a CD123-specific CAR, a CD138-specific CAR, a Kappa. Lambda, B cell maturation agent (BCMA)-specific CAR, a G-protein coupled receptor family C group 5 member D (GPRC5D)-specific CAR, a CD123-specific CAR, a LeY-specific CAR, a NKG2D ligand-specific CAR, a WT1-specific CAR, a GD2-specific CAR, a HER2-specific CAR, a EGFR-specific CAR, a EGFRvIII-specific CAR, a B7H3-specific CAR, a PSMA-specific CAR, a PSCA-specific CAR, a CAIX-specific CAR, a CD171-specific CAR, a CEA-specific CAR, a CSPG4-specific CAR, a EPHA2-specific CAR, a FAP-specific CAR, a FRa-specific CAR, a IL-13Rα-specific CAR, a Mesothelin-specific CAR, a MUC1-specific CAR, a MUC16-specific CAR, a ROR1-specific CAR, a C-Met-specific CAR, a CD133-specific CAR, a Ep-CAM-specific CAR, a GPC3-specific CAR, a HPV16-E6-specific CAR, a IL13Ra2-specific CAR, a MAGEA3-specific CAR, a MAGEA4-specific CAR, a MART1-specific CAR, a NY-ESO-1-specific CAR, a VEGFR2-specific CAR, a α-Folate receptor-specific CAR, a CD24-specific CAR, a CD44v7/8-specific CAR, a EGP-2-specific CAR, a EGP-40-specific CAR, a erb-B2-specific CAR, a erb-B 2,3,4-specific CAR, a FBP-specific CAR, a Fetal acethylcholine e receptor-specific CAR, a GD2-specific CAR, a GD3-specific CAR, a HMW-MAA-specific CAR, a IL-11Rα-specific CAR, a KDR-specific CAR, a Lewis Y-specific CAR, a L1-cell adhesion molecule-specific CAR, a MAGE-A1-specific CAR, a Oncofetal antigen (h5T4)-specific CAR, a TAG-72-specific CAR, or a CD19/CD22-bispecific CAR.
Embodiment 155. The MAIT cell or method according to any one of embodiments 152-154, wherein the CAR comprises a CD19-specific CAR, a CD20-specific CAR, a CD22-specific CAR, a CD38-specific CAR, a CD123-specific CAR, a CD138-specific CAR, a BCMA-specific CAR, or a CD19/CD22-bispecific CAR.
Embodiment 156. The MAIT cell or method according to embodiment 155, wherein the CAR is specific for CD19.
Embodiment 157. The MAIT cell or method according to embodiment 155, wherein the CAR is specific for CD22.
Embodiment 158. The MAIT cell or method according to embodiment 155, wherein the CAR is a CD19/CD22-bispecific CAR.
Embodiment 159. The MAIT cell or method according to embodiment 155, wherein the MAIT cell comprises one or more safety switches.
Embodiment 160. The MAIT cell or method according to any one of embodiments 86-159, wherein the one or more safety switches are capable of controlled killing of the cell or the population of cells.
Embodiment 161. The MAIT cell or method according to embodiment 159 or 160, wherein the one or more safety switches induce controlled cell death in the presence of a drug or prodrug, or upon activation by a selective exogenous compound.
Embodiment 162. The MAIT cell or method according to any one of embodiments 159-161, wherein the one or more safety switches comprise is an inducible protein capable of inducing apoptosis of the cell or the population of cells.
Embodiment 163. The MAIT cell or method according to embodiment 162, wherein the inducible protein capable of inducing apoptosis of the cell or the population of cells is a caspase protein.
Embodiment 164. The MAIT cell or method according to embodiment 163, wherein the caspase protein is caspase 9.
Embodiment 165. The MAIT cell or method according to any one of embodiments 159-164, wherein the one or more safety switches comprise one or more suicide genes.
Embodiment 166. The MAIT cell or method according to embodiment 165, wherein the one or more suicide genes are selected from the group consisting of cytosine deaminase (CyD), herpesvirus thymidine kinase (HSV-Tk), an inducible caspase 9 (iCaspase9), and rapamycin-activated caspase 9 (rapaCasp9).
Embodiment 167. The MAIT cell or method according to any one of embodiments 159-166, wherein the safety switch is an uncloaking system wherein upon activation, cells downregulate expression of immunosuppressive factors and/or upregulate expression of immune signaling molecules thereby reducing the hypoimmunogenicity of the cell.
Embodiment 168. The MAIT cell or method according to any one of embodiments 159-167, wherein at least one of the one or more transgenes are integrated into the genome of the MAIT cell.
Embodiment 169. The MAIT cell or method according to any one of embodiments 159-168, wherein integration is by non-targeted insertion into the genome of the MAIT cell.
Embodiment 170. The MAIT cell or method according to any one of embodiments 159-168, wherein integration is by targeted insertion into a target genomic locus of the MAIT cell.
Embodiment 171. The MAIT cell or method according to embodiment 170, wherein targeted insertion utilizes homology-directed repair.
Embodiment 172. The MAIT cell or method according to embodiment 170 or 171, wherein the target genomic locus is an albumin gene locus, an ABO gene locus, a B2M gene locus, a CIITA gene locus, a CCR5 gene locus, a CD142 gene locus, a CLYBL gene locus, a CXCR4 gene locus, an F3 gene locus, a FUT1 gene locus, an HMGB1 gene locus, a KDM5D gene locus, an LRP1 gene locus, a MIC-A gene locus, a MIC-B gene locus, a PPP1 R12C (also known as AAVS1) gene locus, an RHD gene locus, a ROSA26 gene locus, a safe harbor gene locus, a SHS231 locus, a TAP1 gene locus, a TRAC gene locus, or a TRBC gene locus of the MAIT cell.
Embodiment 173. The MAIT cell or method according to any one of embodiments 159-172, wherein the MAIT cell comprises a genome editing complex.
Embodiment 174. The MAIT cell or method according to embodiment 173, wherein the genome editing complex comprises a genome targeting entity and a genome modifying entity.
Embodiment 175. The MAIT cell or method according to embodiment 174, wherein the genome targeting entity localizes the genome editing complex to the target locus, optionally wherein the genome targeting entity is a nucleic acid-guided targeting entity.
Embodiment 176. The MAIT cell or method according to embodiment 174 or 175, wherein the genome targeting entity comprises a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZF) nucleic acid binding entity, a transcription activator-like effector (TALE) nucleic acid binding entity, a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, or a functional portion thereof.
Embodiment 177. The MAIT cell or method according to any one of embodiments 174-176, wherein the genome targeting entity comprises Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, dCas13b, or a functional portion thereof.
Embodiment 178. The MAIT cell or method according to any one of embodiments 174-177, wherein the genome modifying entity cleaves, deaminates, nicks, polymerizes, interrogates, integrates, cuts, unwinds, breaks, alters, methylates, demethylates, or otherwise destabilizes the target locus.
Embodiment 179. The MAIT cell or method according to any one of embodiments 174-178, wherein the genome modifying entity comprises a recombinase, integrase, transposase, endonuclease, exonuclease, nickase, helicase, DNA polymerase, RNA polymerase, reverse transcriptase, deaminase, flippase, methylase, demethylase, acetylase, a nucleic acid modifying protein, an RNA modifying protein, a DNA modifying protein, an Argonaute protein, an epigenetic modifying protein, a histone modifying protein, or a functional portion thereof.
Embodiment 180. The MAIT cell or method according to any one of embodiments 174-179, wherein the genome modifying entity comprises a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, base editing, prime editing, a Programmable Addition via Site-specific Targeting Elements (PASTE), or a functional portion thereof.
Embodiment 181. The MAIT cell or method according to any one of embodiments 174-180, wherein the genome modifying entity comprises Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, FokI, dCas (D10A), dCas (H840A), dCas13a, dCas13b, a base editor, a prime editor (e.g., a target-primed reverse transcription (TPRT) editor), APOBEC1, cytidine deaminase, adenosine deaminase, uracil glycosylase inhibitor (UGI), adenine base editors (ABE), cytosine base editors (CBE), reverse transcriptase, serine integrase, recombinase, transposase, polymerase, adenine-to-thymine or “ATBE” (or thymine-to-adenine or “TABE”) transversion base editor, ten-eleven translocation methylcytosine dioxygenases (TETs), TET1, TET3, TET1CD, histone acetyltransferase p300, histone methyltransferase SMYD3, histone methyltransferase PRDM9, H3K79 methyltransferase DOT1L, transcriptional repressor, or a functional portion thereof.
Embodiment 182. The MAIT cell or method according to any one of embodiments 174-181, wherein the genome targeting entity and the genome modifying entity are different domains of a single polypeptide.
Embodiment 183. The MAIT cell or method according to any one of embodiments 174-182, wherein the genome editing entity and genome modifying entity are two different polypeptides that are operably linked together.
Embodiment 184. The MAIT cell or method according to any one of embodiments 174-182, wherein the genome editing entity and genome modifying entity are two different polypeptides that are not linked together.
Embodiment 185. The MAIT cell or method according to any one of embodiments 174-184, wherein the genome editing complex comprises a guide nucleic acid having a targeting domain that is complementary to at least one target locus, optionally wherein the guide nucleic acid is a guide RNA (gRNA).
Embodiment 186. The MAIT cell or method according to any one of embodiments 86-185, wherein the MAIT cell is a human cell or an animal cell.
Embodiment 187. The MAIT cell or method according to any one of embodiments 86-186, wherein the MAIT cell is an autologous cell.
Embodiment 188. The MAIT cell or method according to any one of embodiments 86-186, wherein the MAIT cell is an allogeneic cell.
Embodiment 189. The MAIT cell or method according to any one of embodiments 86-188, wherein the reference cell is a comparable cell that does not comprise at least one of the one or more genetic alterations in the MAIT cell, an unmodified MAIT cell, or a wild-type MAIT cell.
Embodiment 190. The MAIT cell or method according to any one of embodiments 86-189, wherein reduced expression comprises no cell surface expression or no detectable cell surface expression.
Embodiment 191. The MAIT cell or method according to any one of embodiments 86-190 wherein reduced expression comprises reduced mRNA expression, optionally wherein reduced expression comprises no detectable mRNA expression.
Embodiment 192. The MAIT cell or method according to any one of embodiments 86-191, wherein reduced expression comprises reduced protein expression or reduced protein activity, optionally wherein reduced expression comprises no detectable protein expression or protein activity.
Embodiment 193. The MAIT cell or method according to any one of embodiments 86-192, wherein one or more genetic alterations comprise:
   (i) one or more indels,
   (ii) one or more frameshift mutations, or
   (iii) one or more knock-outs.
Embodiment 194. The MAIT cell or method according to any one of embodiments 86-193, wherein one or more genetic alterations are made in:
   (i) one or more coding regions, or
   (ii) one or more regulatory regions.
Embodiment 195. The MAIT cell or method according to any one of embodiments 86-194, the MAIT cell is a CAR-T cell.
Embodiment 1%. A population of cells comprising one or more MAIT cells according to any one of embodiments 86-195.
Embodiment 197. A cell therapy product comprising one or more MAIT cells according to any one of embodiments 86-195.
Embodiment 198. A method comprising administering a cell therapy product according to embodiment 197 to a subject.
Embodiment 199. A population of cells according to any one of embodiments 86-195 or a cell therapy product according to embodiment 197 for use in treating a disease or condition.
Embodiment 200. A population of cells according to any one of embodiments 86-195 or a cell therapy product according to embodiment 197 for use in the manufacture of a medicament for treating a disease or condition.
Embodiment 201. Use of a cell therapy product according to embodiment 197 for treating a disease or condition.

### Second Module of Certain Embodiments

1. A method comprising assaying at least one cell parameter of the cell or the population of cells and evaluating a predicted function of the cell or the population of cells based on the at least one cell parameter.
2. A method of profiling the donor capability of a cell or a population of cells for cell therapy, the method comprising assaying at least one cell parameter of the cell or the population of cells and evaluating a predicted function of the cell or the population of cells based on the at least one cell parameter.
3. A method of identifying a cell or a population of cells for making a cell therapy product, the method comprising assaying at least one cell parameter of the cell or the population of cells and evaluating a predicted function of the cell or the population of cells based on the at least one cell parameter.
4. A method of identifying a cell or a population of cells suitable for administration to a subject as a cell therapy, the method comprising assaying at least one cell parameter of the cell or the population of cells and evaluating a predicted function of the cell or the population of cells based on the at least one cell parameter.
5. A method of selecting a cell or a population of cells for making a cell therapy product, the method comprising assaying at least one cell parameter of the cell or the population of cells and evaluating a predicted function of the cell or the population of cells based on the at least one cell parameter.
6. A method of selecting a cell or a population of cells suitable for administration to a subject as a cell therapy, the method comprising assaying at least one cell parameter of the cell or the population of cells and evaluating a predicted function of the cell or the population of cells based on the at least one cell parameter.
7. The method according to any one of embodiments 1-6, wherein the one or more of the cell parameters comprises:
   a) cell activation;
   b) cell polyfunctionality or cell multifunctionality;
   c) cell cytotoxicity;
   d) cell growth rate;
   e) durability of cell growth;
   f) durability of cell response;
   g) the cell's ability to elicit adaptive and innate immune responses;
   h) characteristics associated with a particular cell type (e.g., cell marker characterization, cell cytokine production, antibody production);
   i) cell cytokine production;
   j) cell safety attributes;
   k) cell viability; and/or
   l) cell impurity level(s).
8. The method according to embodiment 7, wherein the safety attributes comprise mycoplasma contamination; sterility; endotoxin level; karyotype; RCL (replication competent lentivirus) detection; VCN (vector copy number); and/or virus screening.
9. A method of profiling the donor capability of a cell or population of cells for cell therapy, the method comprising evaluating the cell or the population of cells for predicted function.
10. A method of identifying a cell or a population of cells for making a cell therapy product, the method comprising evaluating the cell or the population of cells for predicted function.
11. A method of identifying a cell or a population of cells suitable for administration to a subject as a cell therapy, the method comprising evaluating the cell or the population of cells for predicted function.
12. A method of selecting a cell or a population of cells for making a cell therapy product, the method comprising evaluating the cell or the population of cells for predicted function.
13. A method of selecting a cell or a population of cells suitable for administration to a subject as a cell therapy, the method comprising evaluating the cell or the population of cells for predicted function.
14. The method according to any one of the preceding embodiments, wherein evaluating the cell or the population of cells for predicted cell function comprises evaluating the cell or the population of cells for predicted in vivo cell function.
15. The method according to any one of the preceding embodiments, wherein the predicted cell function comprises at least one of the cell functions selected from the group consisting of: cell persistence, engraftment, durability of cell response, potency, cell safety attributes, cell viability, cell impurity level(s), and immunogenicity.
16. The method according to any one of the preceding embodiments, wherein the predicted cell function is evaluated by assaying one or more of cell parameters, optionally the cell parameters comprising:
   a) cell activation;
   b) cell polyfunctionality or cell multifunctionality;
   c) cell cytotoxicity;
   d) cell growth rate;
   e) durability of cell growth;
   f) durability of cell response;
   g) the cell's ability to elicit adaptive and innate immune responses;
   h) characteristics associated with a particular cell type (e.g., cell marker characterization, cell cytokine production, antibody production);
   i) cell cytokine production
   j) cell safety attributes;
   k) cell viability; and/or
   l) cell impurity level(s).
17. The method according to embodiment 16, wherein the safety attributes comprise mycoplasma contamination; sterility; endotoxin level: karyotype; RCL (replication competent lentivirus) detection; VCN (vector copy number); and/or virus screening.
18. The method according to any one of the preceding embodiments, wherein the method comprises assaying at least 2 cell parameters, optionally assaying 2, 3, 4, 5, 6, 7, 8, 9 or 10 cell parameters.
19. The method according to any one of the preceding embodiments, wherein the assaying comprises
   a) an in vitro assay;
   b) an in vivo assay;
   c) an immune assay;
   d) a cell activity assay;
   e) a cell avidity assay;
   f) a cell proliferation assay;
   g) a cell cytotoxicity assay;
   h) a cellular stress assay;
   i) a tumor challenge assay;
   j) an expression assay;
   k) a cytokine production assay;
   l) transcriptomic profiling assay;
   m) a proteomic profiling assay;
   n) a genomic profiling assay;
   o) a genomic stability assay;
   p) an epigenetic profiling assay;
   q) a cell developmental potential profiling assay;
   r) a cell subtyping assay;
   s) a cell receptor profiling assay;
   t) a cell antibody production assay;
   u) a cell antibody profiling assay;
   v) a cell viability assay;
   w) a cell killing assay;
   x) a cytokine dependent growth assay; and/or
   y) a cytokine independent growth assay.
20. The method according to embodiment 19, wherein the immune assay comprises a T cell proliferation assay, a T cell activation assay, a T cell killing assay, an NK cell proliferation assay, an NK cell activation assay, and/or a macrophage activity assay.
21. The method according to any one of the preceding embodiments, wherein the assaying comprises calculating one or more assay readouts, optionally wherein the one or more assay readouts comprise:
   a) a cell functionality score;
   b) a cell polyfunctionality index;
   c) a cell multifunctionality index;
   d) an in vivo efficacy score;
   e) an in vivo activity score;
   f) an in vivo response score;
   g) an in vitro efficacy score;
   h) an in vitro activity score;
   i) an in vitro response score;
   j) an immune efficacy score;
   k) an immune activity score;
   l) an immune response score;
   m) a cell activity score;
   n) a cell activity response score;
   o) a cell specificity score;
   p) a cell sensitivity score;
   q) a cell avidity score;
   r) a cell proliferation score;
   s) a cell proliferative index;
   t) a cell cytotoxicity score;
   u) a cell cytotoxicity response score;
   v) a cell stress score;
   w) a cell stress response score;
   x) a tumor challenge efficacy score;
   y) a tumor challenge activity score;
   z) a tumor challenge response score;
   aa) a tumor challenge specificity score;
   bb) a tumor challenge sensitivity score;
   cc) an expression profile;
   dd)an expression signature;
   ee) an expression signal;
   ff) an expression score;
   gg) a bulk cytokine or chemokine production profile;
   hh) a bulk cytokine or chemokine production signature;
   ii) a bulk cytokine or chemokine production profile;
   jj) a bulk cytokine or chemokine production signal;
   kk) a bulk cytokine or chemokine production score;
   ll) a single cell cytokine or chemokine production profile;
   mm) a single cell cytokine or chemokine production signature;
   nn) a single cell cytokine or chemokine production profile;
   oo) a single cell cytokine or chemokine production signal;
   pp) a single cell cytokine or chemokine production score;
   qq) a transcriptomic profile;
   rr) a transcriptomic signature;
   ss) a transcriptomic signature;
   tt) a transcriptomic score;
   uu) a pathway profile;
   vv) a pathway signature;
   ww) a pathway signal;
   xx) a pathway score;
   yy) a proteomic profile;
   zz) a proteomic signature;
   aaa) a proteomic signal;
   bbb) a proteomic score;
   ccc) a genomic profile;
   ddd) a genomic signature;
   eee) a genomic signal;
   fff) a genomic score;
   ggg) a genomic stability profile;
   hhh) a genomic stability signature;
   iii) a genomic stability signal;
   jjj) a genomic stability score;
   kkk) an epigenetic profile;
   lll) an epigenetic signature;
   mmm) an epigenetic signal;
   nnn) an epigenetic score;
   ooo) a cell developmental potential assessment;
   ppp) a cell developmental potential profile;
   qqq) a cell developmental potential signature;
   rrr) a cell developmental potential score;
   sss) a cell subtyping profile;
   ttt) a cell subtyping signature;
   uuu) a cell subtyping score;
   vvv) a cell receptor profile;
   www) a cell receptor signature;
   xxx) a cell receptor signal;
   yyy) a cell receptor score;
   zzz) a cell antibody production score;
   aaaa) a cell antibody profiling profile;
   bbbb) a cell viability score;
   cccc) a cell viability profile;
   dddd) a cell killing score;
   eeee) a cytokine dependent growth score; and/or
   ffff) a cytokine independent growth score.
22. The method according to any one of the preceding embodiments, wherein the method further comprises determining the cell type of the cell or the cell types in the population of cells, optionally wherein the cell type or cell types are a cell subtype or cell subtypes.
23. The method according to embodiment 22, wherein determining the cell type or the cell types comprise characterizing one or more expressed molecules, optionally wherein the one or more expressed molecules are cell surface molecules.
24. The method according to any one of embodiments 21-23, wherein one or more of the assay readouts are one or more values suitable for use in a processor or a computer-implemented method to evaluate predicted cell function.
25. The method according to embodiment 24, wherein one or more of the assay readouts is a single value.
26. The method according to any one of embodiments 21-25, wherein one or more of the assay readouts are one or more values which require conversion to numerical values.
27. The method according to any one of embodiments 21-25, wherein one or more of the assay readouts are numerical values.
28. The method according to any one of the preceding embodiments, wherein the assaying further comprises using assay readouts and a processor or a computer-implemented method to evaluate predicted cell function.
29. The method according to any one of the preceding embodiments, wherein the evaluation of predicted cell function is provided as a single value.
30. The method according to any one of the preceding embodiments, wherein the evaluating predicted cell function comprises categorising the cell or the population of cells.
31. The method according to any one of the preceding embodiments, wherein the evaluation of predicted cell function is provided as a single value and the cell or the population of cells is categorised based on the value.
32. The method according to any one of the preceding embodiments, wherein the evaluating predicted cell function comprises categorising the cell or the population of cells using a scale for donor capability.
33. The method according to embodiment 32, wherein the cells or the population of cells are categorised based on the number of cells in the population that meet the values in the scale.
34. The method according to embodiment 32 and embodiment 33, wherein the scale comprises 2, 3, 4, 5, 6, 7, 8, 9, 10 or more than 10 points.
35. The method according to any one of the preceding embodiments, wherein the evaluating predicted cell function comprises categorising the cell or the population of cells as having poor, good or exceptional donor capability.
36. The method according to any one of the preceding embodiments, wherein the cells or the population of cells are ranked and categorised as (i) poor if they fall below a reference value, (ii) good if they meet the reference value and (iii) exceptional if they exceed the reference value.
37. The method according to any one of the preceding embodiments, wherein the cells or the population of cells are ranked and categorised as (i) non-useable if they fall below a reference value, and (ii) usable if they meet or exceed the reference value.
38. The method according to any one of the preceding embodiments, wherein the cell or the population of cells is categorised as exceptional or usable if the cell or the population of cells shows ahigh amount of mucosal-associated invariant T (MAIT) relative to a reference cell or population of cells.
39. The method according to any one of the preceding embodiments, wherein the cell or the population of cells is categorised as exceptional or usable if the cell or the population of cells shows a high amount of T Cell Receptor Beta Variable 28 (TRBV28) relative to a reference cell or population of cells.
40. The method according to any one of the preceding embodiments, wherein the cell or the population of cells is categorised as exceptional or usable if the cell or the population of cells shows a high amount of Interleukin-17A (IL17A) relative to a reference cell or population of cells.
41. The method according to any one of the preceding embodiments, wherein the cell or the population of cells is categorised as exceptional or usable if the cell or the population of cells is less activated and has a reduced NK-like signature relative to a reference cell or population of cells.
42. The method according to any one of the preceding embodiments, wherein the cell or the population of cells is categorised as exceptional or usable if the cell or population of cells has an activated phenotype and/or Th1/Tc1 and Th17/Tc17 states.
43. The method according to any one of the preceding embodiments, wherein the evaluating predicted cell function is performed before cell modification, after cell modification, or both before and after cell modification.
44. The method according to any one of the preceding embodiments, wherein the evaluating predicted cell function is performed on the cell or the population of cells prior to any genomic modifications.
45. The method according to any one of the preceding embodiments, wherein the cell or the population of cells are cryopreserved prior to any genomic modifications and evaluating predicted cell function is performed:
   i) before the cell or the population of cells is cryopreserved, and/or
   ii) after the cell or the population of cells has been cryopreserved and thawed.
46. The method according to any one of the preceding embodiments, wherein the cell or the population of cells are stored prior to any genomic modifications and evaluating predicted cell function is performed:
   i) before the cell or the population of cells is stored, and/or
   ii) after the cell or the population of cells has been stored.
47. The method according to any one of the preceding embodiments, wherein the evaluating predicted cell function is performed on the cell or the population of cells prior to any hypoimmunogenic modifications.
48. The method according to any one of the preceding embodiments, wherein the cell or the population of cells are cryopreserved prior to any hypoimmunogenic modifications and the evaluating predicted cell function is performed:
   i) before the cell or the population of cells is cryopreserved, and/or
   ii) after the cell or the population of cells has been cryopreserved and thawed.
49. The method according to any one of the preceding embodiments, wherein the cell or the population of cells are stored prior to any hypoimmunogenic modifications and evaluating predicted cell function is performed:
   i) before the cell or the population of cells is stored, and/or
   ii) after the cell or the population of cells has been stored.
50. The method according to any one of the preceding embodiments, wherein the evaluating predicted cell function is performed after the cell or the population of cells have been modified.
51. The method according to any one of the preceding embodiments, wherein the cell or the population of cells are cryopreserved after any modification and the evaluating predicted cell function is performed:
   i) before the cell or the population of cells is cryopreserved, and/or
   ii) after the cell or the population of cells has been cryopreserved and thawed.
52. The method according to any one of the preceding embodiments, wherein the cell or the population of cells are stored after any modification and evaluating predicted cell function is performed:
   i) before the cell or the population of cells is stored, and/or
   ii) after the cell or the population of cells has been stored.
53. The method according to any one of the preceding embodiments, wherein the evaluating predicted cell function is performed after the cell or the population of cells have completed modifications.
54. The method according to any one of the preceding embodiments, wherein the cell or the population of cells are cryopreserved after completed modification and the evaluating predicted cell function is performed:
   i) before the cell or the population of cells is cryopreserved, and/or
   ii) after the cell or the population of cells has been cryopreserved and thawed.
55. The method according to any one of the preceding embodiments, wherein the cell or the population of cells are stored after completed modification and evaluating predicted cell function is performed:
   i) before the cell or the population of cells is stored, and/or
   ii) after the cell or the population of cells has been stored.
56. The method according to any one of the preceding embodiments, wherein the evaluating predicted cell function is performed before cell differentiation, after cell differentiation, or both before and after cell differentiation.
57. The method according to any one of the preceding embodiments, wherein the evaluating predicted cell function is performed on the cell or the population of cells prior to any differentiation.
58. The method according any one of the preceding embodiments, wherein the cell or the population of cells is cryopreserved prior to any differentiation and the evaluating predicted cell function is performed:
   i) before the cell or the population of cells is cryopreserved, and/or
   ii) after the cell or the population of cells has been cryopreserved and thawed.
59. The method according to any one of the preceding embodiments, wherein the cell or the population of cells are stored prior to any differentiation and evaluating predicted cell function is performed:
   i) before the cell or the population of cells is stored, and/or
   ii) after the cell or the population of cells has been stored.
60. The method according to any one of the preceding embodiments, wherein the evaluating predicted cell function is performed after the cell or the population of cells have been differentiated.
61. The method according to any one of the preceding embodiments, wherein the cell or the population of cells is cryopreserved after differentiation and the evaluating predicted cell function is performed:
   i) before the cell or the population of cells is cryopreserved, and/or
   ii) after the cell or the population of cells has been cryopreserved and thawed.
62. The method according to any one of the preceding embodiments, wherein the cell or the population of cells are stored after differentiation and evaluating predicted cell function is performed:
   i) before the cell or the population of cells is stored, and/or
   ii) after the cell or the population of cells has been stored.
63. The method according to any one of the preceding embodiments, wherein the evaluating predicted cell function is performed after the cell or the population of cells have completed differentiation.
64. The method according to any one of the preceding embodiments, wherein the cell or the population of cells are cryopreserved after completed differentiation and the evaluating predicted cell function is performed:
   i) before the cell or the population of cells is cryopreserved, and/or
   ii) after the cell or the population of cells has been cryopreserved and thawed.
65. The method according to any one of the preceding embodiments, wherein the cell or the population of cells are stored after completed differentiation and evaluating predicted cell function is performed:
   i) before the cell or the population of cells is stored, and/or
   ii) after the cell or the population of cells has been stored.
66. The method according to any one of the preceding embodiments, wherein the evaluating predicted cell function is performed before cell activation, after cell activation, or both before and after cell activation.
67. The method according to any one of the preceding embodiments, wherein the method further comprises identifying the cell or the population of cells as suitable for administration to a subject as a cell therapy if the cell or the population of cells is determined as being ‘good’ or ‘exceptional’ or ‘usable’.
68. The method according to any one of the preceding embodiments, wherein the method further comprises identifying the cell or the population of cells as suitable for making a cell therapy product if the cell or the population of cells is determined as being ‘good’ or ‘exceptional’ or ‘usable’.
69. The method according to any one of the preceding embodiments, wherein further profiling is carried out if the cell or the population of cells is determined as being ‘good’ or ‘usable’.
70. The method according to embodiment 69, wherein the further profiling comprises assaying according to any one of the preceding embodiments at least one of the cell parameters not previously assayed.
71. The method according to embodiment 69, wherein the further profiling comprises assaying according to any one of the preceding embodiments at least one of the same cell parameters previously assayed using an assay that was not previously used.
72. The method according to embodiment 69, wherein the further profiling comprises assaying according to any one of the preceding embodiments at least one of the same cell parameters previously assayed using the assay that was previously used, optionally modifying the experimental parameters of the previously used assay (e.g., the E:T ratio).
73. The method according to any one of embodiments 69-72, wherein the further profiling comprises evaluating predicted cell function according to any one of the preceding embodiments using at least one of the assays as defined in any one of the preceding embodiments, optionally at least 2, 3, 4, 5, 6, 7, 8, 9, 10 or more of the assays.
74. The method according to any one of embodiments 69-73, wherein the cell or the population of cells is identified as suitable for administration to a subject or suitable for making a cell therapy product if the further profiling determines the cell or the population of cells as having an exceptional, good, or usable score.
75. The method according to any one of embodiments 69-74, wherein the cell or the population of cells is identified as suitable for administration to a subject or suitable for making a cell therapy product if addition of the score from the further profiling changes the overall categorisation to exceptional.
76. The method according to any preceding embodiment, wherein the cell or the population of cells is identified as suitable for administration to a subject or suitable for making a cell therapy product if the cell or the population of cells is determined as having a further desirable characteristic.
77. The method according to any preceding embodiment, wherein the cell or the population of cells is identified as suitable for administration to a subject or suitable for making a cell therapy product if the individual from which the donor cells originated has a further desirable characteristic.
78. The method according to embodiment 77, wherein the desirable characteristic is that the individual is female.
79. The method according to embodiment 78, wherein the female is pre-menopausal.
80. The method according to any preceding embodiment, wherein the individual is female.
81. The method according to any preceding embodiment, wherein the individual is between 18 and 35 years old.
82. The method according to any preceding embodiment, wherein the individual is between 18 and 40 years old, 18 and 30 years old, or 18 and 25 years old.
83. The method according to any preceding embodiment, wherein the individual is age 18, 24, 28, 35, or 36.
84. The method according to any preceding embodiment, wherein the individual has a BMI in the range 15-50, 15-45, 15-40, 15-35, 15-30, 15-25, or 15-20.
85. The method according to any preceding embodiment, wherein the individual has a BMI of 22, 24, 34, 39, 41, or 42.
86. The method according to any one of embodiments 76-85, wherein the desirable characteristic is based on the health history of the donor.
87. The method according to any preceding embodiment, wherein the method further comprises identifying cells predicted to have in vivo functionality.
88. The method according to any preceding embodiment, wherein the method further comprises selecting cells predicted to have in vivo functionality.
89. The method according to any preceding embodiment, the assaying comprising:
   a) measuring (i)growth rate and/or (ii) durability of cell growth and/or (iii) durability of cell response in the cell or the population of cells, optionally using a real-time quantitative live-cell analysis platform,
      optionally measuring (i) growth rate and (ii) durability of cell growth and (iii) durability of cell response, optionally using a real-time quantitative live-cell analysis platform, optionally using one or more restimulation cycles;
   b) measuring bulk cytokine production for a panel of cytokines in the cell or the population of cells, optionally using a multiplex cytokine detection technique, an electrochemiluminescence (ECL) detection technique and/or ELISA;
   c) conducting single cell cytokine profiling on the cell or the population of cells, optionally using a multiplex proteomics assay;
   d) conducting gene expression profiling on the cell or the population of cells to determine activation level (i) in a pre-modification resting state of the cell or the population of cells and/or (ii) in a post-modification resting state of the cell or the population of cells and/or (iii) in a post-modification activated state of the cell or the population of cells and/or (iv) in a post tumor challenge exhausted state,
      optionally in a post-modification resting state of the cell or the population of cells;
      optionally using direct digital detection of mRNA molecules of interest; and/or
      optionally conducting gene expression profiling to determine activation level (i) in a pre-modification resting state of the cell or the population of cells and (ii) in a post-modification resting state of the cell or the population of cells and (iii) in a post-modification activated state of the cell or the population of cells and (iv) in a post tumor challenge exhausted state; and/or
   e) measuring the response of the cell or the population of cells in vivo,
      optionally transplanting the cell or the population of cells into an allogeneic host and monitoring for cell activity;
      optionally transplanting the cell or the population of cells into an allogeneic host and monitoring for cell escape from the host immune system;
      optionally transplanting the cell or the population of cells into an allogeneic host and monitoring for cell growth;
      optionally transplanting the cell or the population of cells into an allogeneic host and monitoring for cell rejection:
      optionally transplanting the cell or the population of cells into an allogeneic host and monitoring for cell survival;
      optionally transplanting the cell or the population of cells and a target cell or a population of target cells into an allogeneic host and monitoring for cell activity;
      optionally transplanting the cell or the population of cells and a target cell or a population of target cells into an allogeneic host and monitoring for cell escape from the host immune system;
      optionally transplanting the cell or the population of cells and a target cell or a population of target cells into an allogeneic host and monitoring for cell growth;
      optionally transplanting the cell or the population of cells and a target cell or a population of target cells into an allogeneic host and monitoring for cell rejection;
      optionally transplanting the cell or the population of cells and a target cell or a population of target cells into an allogeneic host and monitoring for cell survival, and/or
      optionally transplanting the cell or the population of cells into an allogeneic host and monitoring for teratoma formation; and/or
   f) measuring the response of a host to the cell or the population of cells,
      optionally transplanting the cell or the population of cells into an allogeneic host, obtaining immune cells from the allogeneic host, and determining an activation state of the immune cells from the allogeneic host; and/or
      optionally transplanting the cell or the population of cells into an allogeneic host,
      obtaining a blood sample from the allogeneic host, and determining a humoral response of the allogeneic host;
   g) cell safety attributes; and/or cell impurity level(s).
90. The method according to embodiment 89, wherein step (a) of the assaying comprises providing a reference value for the measured (i) growth rate and/or (ii) durability of cell growth and/or (iii) durability of cell response and determining whether the measured value(s) is (i) above the reference value; (ii) at the reference value or (iii) below the reference value.
91. The method according to embodiment 89 or embodiment 90, wherein step (b) of the assaying comprises providing a reference value for the bulk cytokine production and determining whether the measured value(s) is (i) above the reference value; (ii) at the reference value or (iii) below the reference value.
92. The method according to any one of embodiments 89-91, wherein step (c) of the assaying comprises calculating a polyfunctionality strength index (PSi) and/or a multifunctional strength index (MSi) from the single cell cytokine profiling, providing a reference value for the PSi and/or MSi and determining whether the calculated value(s) is (i) above the reference value: (ii) at the reference value or (iii) below the reference value, wherein PSi is defined as the signal intensity for cells that produce 2+ cytokines in the population of cells and MSi is defined as the percentage of cells in the population of cells that produce 4+ cytokines.
93. The method according to any one of embodiments 89-92, wherein the assaying comprises (i) identifying cells or the population of cells determined as being at the reference value or above the reference value in any one of steps (a) to (c) and (ii) preparing a signature gene expression profile based on the gene expression profiles of the populations of cells identified in (i).
94. The method according to any one of embodiments 89-93, wherein step (d) of the assaying comprises providing a reference signature gene expression profile and comparing to the gene expression profile of the population of cells.
95. The method according to embodiment 94, wherein the reference signature gene expression profile is an average profile or a normal profile.
96. The method according to any one of embodiments 89-95, wherein step (e) of the assaying comprises (i) monitoring cell survival by bioluminescence imaging (BLI), (ii) determining successful engraftment by determining the surface expression of biomarkers; and/or determining the median area under the curve value (AUC) for the cell growth of the cell or the population of cells or the target cell or the population of target cells.
97. The method according to any one of embodiments 89-%, wherein the method further comprises identifying the cell or the population of cells or considering when identifying the cell or the population of cells as suitable for administration to a subject or suitable for making a cell therapy product if the cell or the population of cells is determined as being above the reference value or at the reference value in any of steps (a) to (c).
98. The method according to any one of embodiments 89-97, wherein the method further comprises identifying the cell or the population of cells or considering when identifying the cell or the population of cells as suitable for administration to a subject or suitable for making a cell therapy product if the gene expression profile of the cell or the population of cells is sufficiently similar to the reference signature gene expression profile, optionally wherein sufficiently similar is at least 60% similar, 70% similar, 80% similar, 90% similar, 95% similar, or 99% similar.
99. The method according to any one of embodiments 89-98, wherein the method further comprises identifying the cell or the population of cells or considering when identifying the cell or the population of cells as suitable for administration to a subject or suitable for making a cell therapy product if (i) the AUC is less than about 20 or between about 20 and 100 at high dose, or (ii) the AUC is less than about 11000 at low dose.
100. The method according to any one of embodiments 89-99, wherein step (a) of the assaying comprises a serial challenge of the cell or the population of cells with a target cell or a population of target cells, optionally wherein the target cell or the population of target cells comprise a tumor cell or a population of tumor cells.
101. The method according to any one of embodiments 89-100, wherein an effector to target (E:T) ratio of about 1:8 or lower is used for the measuring using a real-time quantitative live-cell analysis platform in step (a), optionally wherein the E:T ratio is about 1:8, about 1:9, about 1:10, about 1:12, about 1:15, about 1:16, about 1:18, or about 1:20, and optionally wherein the E:T ratio is about 1:8.
102. The method according to any one of embodiments 89-101, wherein the cell or the population of cells is identified or is considered when identifying the cell or the population of cells as suitable for administration to a subject or suitable for making a cell therapy product if the growth in each one or more restimulation cycles in step (a) is higher than a reference value of growth.
103. The method according to any one of embodiments 89-102, wherein the cell or the population of cells is identified or is considered when identifying the cell or the population of cells as suitable for administration to a subject or suitable for making a cell therapy product if the growth in each one or more restimulation cycles in step (a) is higher than about 0.2 fold more than a reference value of growth, optionally higher than about 4 fold.
104. The method according to any one of embodiments 89-103, wherein the cell or the population of cells is identified or is considered when identifying the cell or the population of cells as suitable for administration to a subject or suitable for making a cell therapy product if the durability of cell growth in step (a) is higher than a reference value of durability of cell growth.
105. The method according to any one of embodiments 89-104, wherein the cell or the population of cells is identified or is considered when identifying the cell or the population of cells as suitable for administration to a subject or suitable for making a cell therapy product if the durability of cell growth in step (a)is at least about 5% higher than a reference value of durability of cell growth, optionally at least about 10% higher.
106. The method according to any one of embodiments 89-105, wherein the cell or the population of cells is identified or is considered when identifying the cell or the population of cells as suitable for administration to a subject or suitable for making a cell therapy product if the durability of cell growth in step (a) is 0 or higher when durability of cell growth is measured as a slope of the fold change of the cell or the population of cells over a course of the serial challenge, optionally wherein the assaying is performed using an IncuCyte assay.
107. The method according to any one of embodiments 89-106, wherein the cell or the population of cells is identified or is considered when identifying the cell or the population of cells as suitable for administration to a subject or suitable for making a cell therapy product if the durability of cell response in step (a) is higher than a reference durability of cell response.
108. The method according to any one of embodiments 89-107, wherein the cell or the population of cells is identified or is considered when identifying the cell or the population of cells as suitable for administration to a subject or suitable for making a cell therapy product if the durability of cell response in step (a) is 0 or lower when durability of cell response is measured as a slope of the fold change of the target cell or the population of target cells over a course of the serial challenge, optionally wherein the assaying is performed using an IncuCyte assay.
109. The method according to any one of embodiments 89-108, wherein the panel of cytokines measured in step (b) comprises GM-CSF, GzmA, GzmB, IFN-g, TNF-a, IL-2, IL-6, IL-17A, IL-1b, IL-1RA, or any combinations thereof.
110. The method according to any one of embodiments 89-109, wherein the panel of cytokines measured in step (b) comprises IL-17A.
111. The method according to embodiment 110, wherein the panel of cytokines measured in step (b) further comprises at least one of GM-CSF, GzmA, GzmB, IFN-g, TNF-a, IL-2, IL-6, IL-1b, or IL-1RA.
112. The method according to any one of embodiments 89-111, wherein an E:T ratio of about 1:1, about 1:2, about 1:3, about 1:4, about 1:5, about 1:6, about 1:7, about 1:8, about 1:9, about 1:10, about 1:12, about 1:15, about 1:16, or about 1:20 is used for the measuring cytokine production in step (b).
113. The method according to any one of embodiments 89-112, wherein an E:T ratio of about 1:2 is used for the measuring cytokine production in step (b).
114. The method according to any one of embodiments 89-113, wherein the cell or the population of cells is identified or is considered when identifying the cell or the population of cells as suitable for administration to a subject or suitable for making a cell therapy product if the measured cytokine production in step (b) is higher than the median of the average cytokine production values calculated for the more than one reference cell population, optionally about 5% or more.
115. The method according to any one of embodiments 89-114, wherein the cell or the population of cells is identified or is considered when identifying the cell or the population of cells as suitable for administration to a subject or suitable for making a cell therapy product if the measured cytokine production in step (b) is at least about 10% higher than the median of the average cytokine production values calculated for the more than one reference cell population.
116. The method according to any one of embodiments 89-115, wherein bulk cytokine production in step (b) is measured at an E:T ratio of 1:2 and the cell or the population of cells is identified or is considered when identifying the cell or the population of cells as suitable for administration to a subject or suitable for making a cell therapy product if bulk cytokine production is measured as about 1.0 pg per cell or higher, such as about 1.5 pg per cell or higher, or about 1.9 pg per cell or higher.
117. The method according to any one of embodiments 89-116, wherein a polyfunctionality strength index (PSi) is defined from the single cell cytokine profiling in step (c) based on the signal intensity for cells that produce 2+ cytokines in the population of cells.
118. The method according to any one of embodiments 89-117, wherein the cell or the population of cells is identified or is considered when identifying the cell or the population of cells as suitable for administration to a subject or suitable for making a cell therapy product if the polyfunctionality strength index (PSi) in step (c) is higher than a reference value.
119. The method according to any one of embodiments 89-118, wherein the cell or the population of cells is identified or is considered when identifying the cell or the population of cells as suitable for administration to a subject or suitable for making a cell therapy product if the polyfunctionality strength index (PSi) in step (c) is 90 or above, such as 100 or above, or 150 or above, or 200 or above.
120. The method according to any one of embodiments 89-119, wherein the cell or the population of cells is identified or is considered when identifying the cell or the population of cells as suitable for administration to a subject or suitable for making a cell therapy product if the polyfunctionality strength index (PSi) in step (c) is higher than the median polyfunctionality strength index (PSi) of more than one reference cell population, optionally 5% or more.
121. The method according to any one of embodiments 89-120, wherein the cell or the population of cells is identified or is considered when identifying the cell or the population of cells as suitable for administration to a subject or suitable for making a cell therapy product if the polyfunctionality strength index (PSi) in step (c) is at least 10% higher than the median polyfunctionality strength index (PSi) of more than one reference cell population.
122. The method according to any one of embodiments 89-121, wherein the cell or the population of cells is identified or is considered when identifying the cell or the population of cells as suitable for administration to a subject or suitable for making a cell therapy product if the polyfunctionality strength index (PSi) in step (c) is at least 15% higher than the median polyfunctionality strength index (PSi) of more than one reference cell population.
123. The method according to any one of embodiments 89-122, wherein a multi-functionality strength index (MSi) is defined from the single cell cytokine profiling in step (c) for the percentage of cells in the population of cells that produce 4+ cytokines.
124. The method according to any one of embodiments 89-123, wherein the cell or the population of cells is identified or is considered when identifying the cell or the population of cells as suitable for administration to a subject or suitable for making a cell therapy product if the multi-functionality strength index (MSi) in step (c) is higher than the reference multi-functionality strength index (MSi).
125. the method according to any one of embodiments 89-124, wherein the cell or the population of cells is identified or is considered when identifying the cell or the population of cells as suitable for administration to a subject or suitable for making a cell therapy product if the multi-functionality strength index (MSi) in step (c) is 0.1% or higher, such as 0.2% or higher or 0.3% or higher.
126. The method according to any one of embodiments 89-125, wherein the cell or the population of cells is identified or is considered when identifying the cell or the population of cells as suitable for administration to a subject or suitable for making a cell therapy product as a cell therapy if, in the pre-modification resting state, the cell or cell population
   i. has a less NK-like signature than the reference signature gene expression profile, optionally wherein the NK-like signature comprises expression of CD160, GNLY, GZMH, KLRK1, NKG7, CD56, KIR3RD and/or KLRD1;
   ii. has a less activated T cell state signature than the reference signature gene expression profile, optionally wherein the activated cell state signature comprises expression of FASLG, GZMB, II2RB, I112RB, TIGIT, Tim-3, GITR and/or CD38;
   iii. has decreased expression of negative regulators of viral mRNA translation than the reference signature gene expression profile, optionally wherein the negative regulators of viral mRNA translation comprise IFIT1, IFIT3, OASL, OAS1 and/or OAS3;
   iv. has a greater naïve (TN) and/or central memory (CM) T cell phenotype signature than the reference signature gene expression profile, optionally wherein the cell phenotype comprises expression of CD9 and/or PEACAM(CD31); and/or
   v. has greater TCR clonality signature differences than the reference signature gene expression profile, optionally wherein the clonality signature differences comprise higher levels of TRBV28-public canonical MR1-restricted cells/mucosal-associated invariant (MAIT) cells.
127. The method according to any one of embodiments 89-125, wherein the cell or the population of cells is identified or is considered when identifying the cell or the population of cells as suitable for administration to a subject or suitable for making a cell therapy product if, in the post-modification resting state, the cell or cell population
   i. has reduced expression of Th2/Tc2 signature genes than the reference signature gene expression profile, optionally wherein the Th2/Tc2 signature comprises expression of IL4, IL5 and/or IL13;
   ii. has a reduced naïve (TN) and/or central memory (CM) T cell phenotype signature than the reference signature gene expression profile, optionally wherein the cell phenotype signature comprises expression of CD9, CD45RA and/or PEACAM(CD31);
   iii. has greater expression of Th1/Tc1 signature genes than the reference signature gene expression profile, optionally wherein the Th1/Tc1 signature comprises expression of IRF8, IRF1 and/or IFNG;
   iv. has greater expression of T17 signature genes than the reference signature gene expression profile, optionally wherein the Th17 signature comprises expression of IL22 and/or IL26; and/or
   v. has greater TCR diversity signature differences than the reference signature gene expression profile, optionally wherein the diversity signature differences comprise higher levels of TRBV28-public canonical MR1-restricted cells/mucosal-associated invariant (MAIT) cells.
128. The method according to any one of embodiments 89-125, wherein the cell or the population of cells is identified or is considered when identifying the cell or the population of the cells as suitable for administration to a subject or suitable for making a cell therapy product if, in the post-modification activated state, the cell or cell population
   i. has reduced expression of Th2/Tc2 signature genes than the reference signature gene expression profile, optionally wherein the Th2/Tc2 signature comprises expression of IL5;
   ii. has a reduced naïve (TN) and/or central memory (CM) T cell phenotype signature than the reference signature gene expression profile, optionally wherein the cell phenotype signature comprises expression of PEACAM(CD31);
   iii. has greater expression of Th1/Tc1 signature genes than the reference signature gene expression profile, optionally wherein the Th1/Tc1 signature comprises expression of IRF1 and/or IFNG;
   iv. has greater expression of Th17/Tc17 signature genes than the reference signature gene expression profile, optionally wherein the Th17/Tc17 signature comprises expression of IL22, I117F and/or IL26;
   v. has greater TCR diversity signature differences than the reference signature gene expression profile, optionally wherein the diversity signature differences comprise higher levels of TRBV28, KLRB1, MAIT cells; and/or
   vi. has greater T cell activation/stimulation signature than the reference signature gene expression profile, optionally wherein the cell activation/stimulation signature comprises expression of ICOS, OX40, Lag3, GITR, VISTA, CD40L, CTLA4 and/or 4-1BB.
129. The method according to any one of embodiments 89-125, wherein the cell or the population of cells is identified or is considered when identifying the cell or the population of cells as suitable for administration to a subject or suitable for making a cell therapy product if, in the post-tumor challenge exhausted state, the cell or cell population
   i. has reduced expression of CREB1 and/or SOCS4 (inhibitor of NFAT TF and TCR signalling) than the reference signature gene expression profile; and/or
   ii. has greater expression of FOS (part of NFAT TF) and/or MTCP1 (enhancer of AKT signalling) than the reference signature gene expression profile.
130. The method according to any one of embodiments 89-129, wherein the method comprises at least step (a).
131. The method according to any one of embodiments 89-129, wherein the method comprises at least step (b).
132. The method according to any one of embodiments 89-129, wherein the method comprises at least step (c).
133. The method according to any one of embodiments 89-129, wherein the method comprises at least step (d).
134. The method according to any one of embodiments 89-129, wherein the method comprises at least step (e).
135. The method according to any one of embodiments 89-129, wherein the method comprises at least step (f).
136. The method according to any one of embodiments 89-129, wherein the method comprises at least step (g).
137. The method according to any one of embodiments 89-129, wherein the method comprises at least step (a) and step (b).
138. The method according to any one of embodiments 89-129, wherein the method comprises at least step (a) and step (c).
139. The method according to any one of embodiments 89-129, wherein the method comprises at least step (a) and step (d).
140. The method according to any one of embodiments 89-129, wherein the method comprises at least step (a) and step (e).
141. The method according to any one of embodiments 89-129, wherein the method comprises at least step (a) and step (f).
142. The method according to any one of embodiments 89-129, wherein the method comprises at least step (a) and step (g).
143. The method according to any one of embodiments 89-129, wherein the method comprises at least step (b) and step (c).
144. The method according to any one of embodiments 89-129, wherein the method comprises at least step (b) and step (d).
145. The method according to any one of embodiments 89-129, wherein the method comprises at least step (b) and step (e).
146. The method according to any one of embodiments 89-129, wherein the method comprises at least step (b) and step (f).
147. The method according to any one of embodiments 89-129, wherein the method comprises at least step (b) and step (g).
148. The method according to any one of embodiments 89-129, wherein the method comprises at least step (c) and step (d).
149. The method according to any one of embodiments 89-129, wherein the method comprises at least step (c) and step (e).
150. The method according to any one of embodiments 89-129, wherein the method comprises at least step (c) and step (f).
151. The method according to any one of embodiments 89-129, wherein the method comprises at least step (c) and step (g).
152. The method according to any one of embodiments 89-129, wherein the method comprises at least step (d) and step (e).
153. The method according to any one of embodiments 89-129, wherein the method comprises at least step (d) and step (f).
154. The method according to any one of embodiments 89-129, wherein the method comprises at least step (d) and step (g).
155. The method according to any one of embodiments 89-129, wherein the method comprises at least step (f) and step (g).
156. The method according to any one of embodiments 89-129, wherein the method comprises at least step (a), step (b) and step (c).
157. The method according to any one of embodiments 89-129, wherein the method comprises at least step (a), step (b) and step (d).
158. The method according to any one of embodiments 89-129, wherein the method comprises at least step (a), step (b) and step (e).
159. The method according to any one of embodiments 89-129, wherein the method comprises at least step (a), step (b) and step (f).
160. The method according to any one of embodiments 89-129, wherein the method comprises at least step (a), step (c) and step (d).
161. The method according to any one of embodiments 89-129, wherein the method comprises at least step (a), step (c) and step (e).
162. The method according to any one of embodiments 89-129, wherein the method comprises at least step (a), step (c) and step (f).
163. The method according to any one of embodiments 89-129, wherein the method comprises at least step (a), step (d) and step (e).
164. The method according to any one of embodiments 89-129, wherein the method comprises at least step (a), step (d) and step (f).
165. The method according to any one of embodiments 89-129, wherein the method comprises at least step (a), step (e) and step (f).
166. The method according to any one of embodiments 89-129, wherein the method comprises at least step (b), step (c) and step (d).
167. The method according to any one of embodiments 89-129, wherein the method comprises at least step (b), step (c) and step (e).
168. The method according to any one of embodiments 89-129, wherein the method comprises at least step (b), step (c) and step (f).
169. The method according to any one of embodiments 89-129, wherein the method comprises at least step (b), step (d) and step (e).
170. The method according to any one of embodiments 89-129, wherein the method comprises at least step (b), step (d) and step (f).
171. The method according to any one of embodiments 89-129, wherein the method comprises at least step (b), step (e) and step (f).
172. The method according to any one of embodiments 89-129, wherein the method comprises at least step (c), step (d) and step (e).
173. The method according to any one of embodiments 89-129, wherein the method comprises at least step (c), step (d) and step (f).
174. The method according to any one of embodiments 89-129, wherein the method comprises at least step (c), step (e) and step (f).
175. The method according to any one of embodiments 89-129, wherein the method comprises at least step (a), step (b), step (c) and step (d).
176. The method according to any one of embodiments 89-129, wherein the method comprises at least step (a), step (b), step (c) and step (e).
177. The method according to any one of embodiments 89-129, wherein the method comprises at least step (a), step (b), step (c) and step (f).
178. The method according to any one of embodiments 89-129, wherein the method comprises at least step (a), step (b), step (d) and step (e).
179. The method according to any one of embodiments 89-129, wherein the method comprises at least step (a), step (b), step (d) and step (f).
180. The method according to any one of embodiments 89-129, wherein the method comprises at least step (a), step (c), step (d) and step (e).
181. The method according to any one of embodiments 89-129, wherein the method comprises at least step (a), step (c), step (d) and step (f).
182. The method according to any one of embodiments 89-129, wherein the method comprises at least step (a), step (d), step (e) and step (f).
183. The method according to any one of embodiments 89-129, wherein the method comprises at least step (b), step (c), step (d) and step (e).
184. The method according to any one of embodiments 89-129, wherein the method comprises at least step (b), step (c), step (d) and step (f).
185. The method according to any one of embodiments 89-129, wherein the method comprises at least step (b), step (d), step (e) and step (f).
186. The method according to any one of embodiments 89-129, wherein the method comprises at least step (c), step (d), step (e) and step (f).
187. The method according to any one of embodiments 89-129, wherein the method comprises step (a), step (b), step (c), step (d) and step (e).
188. The method according to any one of embodiments 89-129, wherein the method comprises step (a), step (b), step (c), step (d) and step (f).
189. The method according to any one of embodiments 89-129, wherein the method comprises step (a), step (c), step (d), step (e) and step (f).
190. The method according to any one of embodiments 89-129, wherein the method comprises step (a), step (b), step (c), step (d), step (e) and step (f).
191. The method according to any one of embodiments 89-190, wherein the host is a mammal.
192. The method according to any one of embodiments 89-191, wherein the host is murine.
193. The method according to any one of embodiments 89-192, wherein the host is a humanized model.
194. The method according to any one of embodiments 89-193, wherein the host is an allogeneic humanized immunodeficient mouse model, optionally wherein the host is an allogeneic humanized NSG-SGM3 mouse.
195. The method according to any preceding embodiment, wherein the method further comprises administering the cell or the population of cells to a subject.
196. The method according to any preceding embodiment, wherein:
   i. the population includes cells with hypoimmune gene modifications (HIP cells) that exhibit reduced expression of one or more molecules of the MHC class I and/or MHC class II molecules and optionally also exhibit increased expression of at least one tolerogenic factor; and
   ii. at least 30%, at least 40%, at least 50%, at least 60%, at least 70%, at least 80%, or at least 90% of cells in the population exhibit reduced expression of one or more molecules of the MHC class I and/or MHC class II molecules and optionally also exhibit increased expression of at least one tolerogenic factor.
197. The method according to embodiment 196, wherein at least 5%, at least 10%, at least 20%, at least 30%, at least 40%, at least 50%, at least 60%, at least 70% of cells in the population of cells do not exhibit reduced expression of one or more molecules of the MHC class I and/or MHC class II molecules and optionally also do not exhibit increased expression of at least one tolerogenic factor.
198. The method according to any preceding embodiment, wherein 30-90%, 30-80%, 30-70%, 30-60%, 30-50% or 40-50% of cells in the population of cells are HIP cells that exhibit reduced expression of one or more molecules of the MHC class I and/or MHC class II molecules and optionally increased expression of at least one tolerogenic factor.
199. The method according to any preceding embodiment, wherein 70-100%, 80-100%, or 90-100% of cells in the population of cells express a CAR.
200. The method according to any one of embodiments 195-199, wherein the subject is in need of therapy.
201. The method according to any one of embodiments 195-200, wherein the subject has or is suspected of having a cellular deficiency (optionally diabetes, cancer, vascularization disorders, ocular disease, thyroid disease, skin diseases, and liver diseases), a condition or disease associated with a vascular condition or disease; a vascular condition or disease; a condition or disease associated with autoimmune thyroiditis; autoimmune thyroiditis; a condition or disease associated with a liver disease; liver disease (optionally cirrhosis of the liver); a condition or disease associated with a corneal disease: corneal disease (optionally Fuchs dystrophy or congenital hereditary endothelial dystrophy); a condition or disease associated with a kidney disease; kidney disease.; a disease associated with cancer; cancer (optionally B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, or bladder cancer); a condition or disease associated with a hematopoietic disease or disorder; a hematopoietic disease or disorder (optionally myelodysplasia, aplastic anemia, Fanconi anemia, paroxysmal nocturnal hemoglobinuria, Sickle cell disease, Diamond Blackfan anemia, Schachman Diamond disorder, Kostmann's syndrome, chronic granulomatous disease, adrenoleukodystrophy, leukocyte adhesion deficiency, hemophilia, thalassemia, beta-thalassemia, leukaemia such as acute lymphocytic leukemia (ALL), acute myelogenous (myeloid) leukemia (AML), adult lymphoblastic leukaemia, chronic lymphocytic leukemia (CLL), B-cell chronic lymphocytic leukemia (B-CLL), chronic myeloid leukemia (CML), juvenile chronic myelogenous leukemia (CML), and juvenile myelomonocytic leukemia (JMML), severe combined immunodeficiency disease (SCID), X-linked severe combined immunodeficiency. Wiskott-Aldrich syndrome (WAS), adenosine-deaminase (ADA) deficiency, chronic granulomatous disease, Chediak-Higashi syndrome, Hodgkin's lymphoma, non-Hodgkin's lymphoma (NHL) or AIDS); a condition or disease associated with leukemia or myeloma: leukemia; myeloma; a condition or disease associated with an autoimmune disease or condition; an autoimmune disease or condition (optionally acute disseminated encephalomyelitis, acute hemorrhagic leukoencephalitis, Addison's disease, Agammaglobulinemia, Alopecia areata, amyotrophic lateral sclerosis, ankylosing spondylitis, antiphospholipid syndrome, antisynthetase syndrome, atopic allergy, autoimmune aplastic anemia, autoimmune cardiomyopathy, autoimmune enteropathy, autoimmune hemolytic anemia, autoimmune hepatitis, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticaria, autoimmune uveitis, Balo disease, Balo concentric sclerosis, Bechets syndrome, Berger's disease, Bickerstaff's encephalitis, Blau syndrome, bullous pemphigoid, cancer, Castleman's disease, celiac disease, chronic inflammatory demyelinating polyneuropathy, chronic recurrent multifocal osteomyelitis. Churg-Strauss syndrome, cicatricial pemphigoid, Cogan syndrome, cold agglutinin disease, complement component 2 deficiency, cranial arteritis, CREST syndrome, Crohn's disease, Cushing's syndrome, cutaneous leukocytoclastic angiitis, Dego's disease, Dercum's disease, dermatitis herpetiformis, dermatomyositis, diabetes mellitus type 1, diffuse cutaneous systemic sclerosis, Dressler's syndrome, discoid lupus erythematosus, eczema, enthesitis-related arthritis, eosinophilic fasciitis, eosinophilic gastroenteritis, epidermolysis bullosa acquisita, erythema nodosum, essential mixed cryoglobulinemia, Evan's syndrome, firodysplasia ossificans progressiva, fibrosing aveolitis, gastritis, gastrointestinal pemphigoid, giant cell arteritis, glomerulonephritis, goodpasture's syndrome, Grave's disease, Guillain-Barre syndrome (GBS), Hashimoto's encephalitis, Hashimoto's thyroiditis, hemolytic anaemia, Henoch-Schonlein purpura, herpes gestationis, hypogammaglobulinemia, idiopathic inflammatory demyelinating disease, idiopathic pulmonary fibrosis, idiopathic thrombocytopenic purpura, IgA nephropathy, inclusion body myositis, inflammatory demyelinating polyneuropathy, interstitial cystitis, juvenile idiopathic arthritis, juvenile rheumatoid arthritis, Kawasaki's disease, Lambert-Eaton myasthenic syndrome, leukocytoclastic vasculitis, lichen planus, lichen sclerosus, linear IgA disease (LAD), Lou Gehrig's disease, lupoid hepatitis, lupus erythematosus, Majeed syndrome, Meniere's disease, microscopic polyangiitis, Miller-Fisher syndrome, mixed connective tissue disease, morphea, Mucha-Habermann disease, multiple sclerosis, myasthenia gravis, myositis, neuropyelitis optica, neuromyotonia, ocular cicatricial pemphigoid, opsoclonus myoclonus syndrome, ord thyroiditis, palindromic rheumatism, paraneoplastic cerebellar degeneration, paroxysmal nocturnal hemoglobinuria (PNH), Parry Romberg syndrome, Parsonnage-Turner syndrome, pars planitis, pemphigus, pemphigus vulgaris, permicious anemia, perivenous encephalomyelitis, POEMS syndrome, polyarteritis nodosa, polymyalgia rheumatica, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restless leg syndrome, retroperitoneal fibrosis, rheumatoid arthritis, rheumatoid fever, sarcoidosis, Schmidt syndrome, Schnitzler syndrome, scleritis, scleroderma, Sjogren's syndrome, spondylarthropathy, Still's disease, stiff person syndrome, subacute bacterial endocarditis, Susac's syndrome, Sweet's syndrome, Sydenham chorea, sympathetic ophthalmia, Takayasu's arteritis, temporal arteritis, Tolosa-Hunt syndrome, transverse myelitis, ulcerative colitis, undifferentiated connective tissue disease, undifferentiated spondylarthropathy, vasculitis, vitiligo or Wegener's granulomatosis); a condition or disease associated with Parkinson's disease. Huntington disease, multiple sclerosis, a neurodegenerative disease or condition, attention deficit hyperactivity disorder (ADHD), Tourette Syndrome (TS), schizophrenia, psychosis, depression, a neuropsychiatric disorder stroke, or amyotrophic lateral sclerosis (ALS); Parkinson's disease; Huntington disease; multiple sclerosis; a neurodegenerative disease or condition; attention deficit hyperactivity disorder (ADHD): Tourette Syndrome (TS); schizophrenia, psychosis, depression; a neuropsychiatric disorder stroke: or amyotrophic lateral sclerosis (ALS).
202. The method according to any one of embodiments 1-201, wherein the cell or the population of cells is cryopreserved.
203. The method according to any one of embodiments 1-201, wherein the cell or the population of cells are thawed.
204. The method according to any one of embodiments 1-201, wherein the functional assays are performed before freezing.
205. The method according to any one of embodiments 1-201, the method further comprising thawing the cells.
206. The method according to any one of embodiments 1-201, wherein the functional assays are performed after thawing.
207. The method according to any one of embodiments 1-201, wherein the cell or the population of cells is not cryopreserved.
208. The method according to any one of embodiments 202-206, wherein after thawing the at least 30%, at least 40%, at least 50%, at least 60%, at least 70%, at least 80%, or at least 90% of cells in the population that exhibit reduced expression of one or more molecules of the MHC class I and/or MHC class II molecules and optionally also exhibit increased expression of at least one tolerogenic factor are viable cells.
209. The method according to any one of embodiments 202-206, wherein after thawing the at least 30%, at least 40%, at least 50%, at least 60%, at least 70%, at least 80%, or at least 90% of cells in the population that exhibit reduced expression of one or more molecules of the MHC class I and/or MHC class II molecules and optionally also exhibit increased expression of at least one tolerogenic factor comprise a mixture of viable and non-viable cells.
210. The method according to any one of embodiments 1-209, wherein the cell or the population of cells are unmodified.
211. The method according to any one of embodiments 1-209, wherein the cell or the population of cells are modified.
212. The method according to any preceding embodiment, wherein the cell or the population of cells are hypoimmunogenic.
213. The method according to any preceding embodiment, wherein the cell or the population of cells has been modified to be hypoimmunogenic.
214. The method according to any preceding embodiment, wherein the evaluating predicted cell function is performed on the cell or the population of cells prior to any hypoimmunogenic modifications.
215. The method according to any one of the preceding embodiments, wherein the cell or the population of cells are cryopreserved prior to any hypoimmunogenic modifications and the evaluating predicted cell function is performed:
   i) before the cell or the population of cells is cryopreserved, and/or
   ii) after the cell or the population of cells has been cryopreserved and thawed.
216. The method according to any one of the preceding embodiments, wherein the cell or the population of cells are stored prior to any hypoimmunogenic modifications and evaluating predicted cell function is performed:
   i) before the cell or the population of cells is stored, and/or
   ii) after the cell or the population of cells has been stored.
217. The method according to any one of the preceding embodiments, wherein the evaluating predicted cell function is performed on the cell or the population of cells following introduction of one or more hypoimmune gene modifications.
218. The method according to any one of the preceding embodiments, wherein the cell or the population of cells are cryopreserved following introduction of one or more hypoimmune gene modifications and the evaluating predicted cell function is performed:
   i) before the cell or the population of cells is cryopreserved, and/or
   ii) after the cell or the population of cells has been cryopreserved and thawed.
219. The method according to any one of the preceding embodiments, wherein the cell or the population of cells are stored following introduction of one or more hypoimmune gene modifications and evaluating predicted cell function is performed:
   i) before the cell or the population of cells is stored, and/or
   ii) after the cell or the population of cells has been stored.
220. The method according to any one of the preceding embodiments, wherein the evaluating predicted cell function is performed on the cell or the population of cells following introduction of all hypoimmune gene modifications.
221. The method according to any one of the preceding embodiments, wherein the cell or the population of cells are cryopreserved following introduction of all hypoimmune gene modifications and the evaluating predicted cell function is performed:
   i) before the cell or the population of cells is cryopreserved, and/or
   ii) after the cell or the population of cells has been cryopreserved and thawed.
222. The method according to any one of the preceding embodiments, wherein the cell or the population of cells are stored following introduction of all hypoimmune gene modifications and evaluating predicted cell function is performed:
   i) before the cell or the population of cells is stored, and/or
   ii) after the cell or the population of cells has been stored.
223. The method according to any one of the preceding embodiments, wherein the evaluating predicted cell function is performed before cell differentiation, after cell differentiation, or both before and after cell differentiation.
224. The method according to any one of the preceding embodiments, wherein the evaluating predicted cell function is performed on the cell or the population of cells prior to any differentiation.
225. The method according any one of the preceding embodiments, wherein the cell or the population of cells is cryopreserved prior to any differentiation and the evaluating predicted cell function is performed:
   i) before the cell or the population of cells is cryopreserved, and/or
   ii) after the cell or the population of cells has been cryopreserved and thawed.
226. The method according to any one of the preceding embodiments, wherein the cell or the population of cells are stored prior to any differentiation and evaluating predicted cell function is performed:
   i) before the cell or the population of cells is stored, and/or
   ii) after the cell or the population of cells has been stored.
227. The method according to any one of the preceding embodiments, wherein the evaluating predicted cell function is performed after the cell or the population of cells have been differentiated.
228. The method according to any one of the preceding embodiments, wherein the cell or the population of cells is cryopreserved after differentiation and the evaluating predicted cell function is performed:
   i) before the cell or the population of cells is cryopreserved, and/or
   ii) after the cell or the population of cells has been cryopreserved and thawed.
229. The method according to any one of the preceding embodiments, wherein the cell or the population of cells are stored after differentiation and evaluating predicted cell function is performed:
   i) before the cell or the population of cells is stored, and/or
   ii) after the cell or the population of cells has been stored.
230. The method according to any one of the preceding embodiments, wherein the evaluating predicted cell function is performed after the cell or the population of cells have completed differentiation.
231. The method according to any one of the preceding embodiments, wherein the cell or the population of cells are cryopreserved after completed differentiation and the evaluating predicted cell function is performed:
   i) before the cell or the population of cells is cryopreserved, and/or
   ii) after the cell or the population of cells has been cryopreserved and thawed.
232. The method according to any one of the preceding embodiments, wherein the cell or the population of cells are stored after completed differentiation and evaluating predicted cell function is performed:
   i) before the cell or the population of cells is stored, and/or
   ii) after the cell or the population of cells has been stored.
233. The method according to any one of the preceding embodiments, wherein the cell or population of cells that are cryopreserved or have been cryopreserved comprise decreased expression of one or more MHC class I molecules and/or one or more MHC class II molecules.
234. The method according to any one of the preceding embodiments, wherein the cell or population of cells that are cryopreserved or have been cryopreserved comprise increased expression of one or more tolerogenic factors.
235. The method according to any one of the preceding embodiments, wherein the cell or population of cells that are cryopreserved or have been cryopreserved comprise decreased expression of one or more MHC class I molecules and increased expression of one or more tolerogenic factors.
236. The method according to any one of the preceding embodiments, wherein the cell or population of cells that are cryopreserved or have been cryopreserved comprise decreased expression of one or more MHC class II molecules and increased expression of one or more tolerogenic factors.
237. The method according to any one of the preceding embodiments, wherein the cell or population of cells that are cryopreserved or have been cryopreserved comprise decreased expression of one or more MHC class I molecules, one or more MHC class II molecules, and increased expression of one or more tolerogenic factors.
238. The method according to any one of the preceding embodiments, wherein the cell or population of cells that are cryopreserved or have been cryopreserved comprise undifferentiated cells.
239. The method according to any one of the preceding embodiments, wherein the cell or population of cells that are cryopreserved or have been cryopreserved comprise cells that have been differentiated to an intermediate cell type.
240. The method according to any one of the preceding embodiments, wherein the cell or population of cells that are cryopreserved or have been cryopreserved comprise cells that have been differentiated to a fully differentiated cell type.
241. The method according to any one of the preceding embodiments, wherein the at least one cell parameter comprises determining hypoimmunity of the cell or the population of cells (e.g., using the XCelligence cell proliferation assay).
242. The method according to any one of the preceding embodiments, wherein the at least one cell parameter comprises determining CD47 expression (e.g., using flow cytometry) (e.g., about 1×, about 2×, about 3×, about 4×, about 5×, or more CD47 expression over baseline).
243. The method according to any one of embodiments 212-242, wherein the hypoimmunogenic cells have reduced expression of B2M, CIITA and TCRalpha and increased expression of CD47.
244. The method according to any preceding embodiment, wherein the method further comprises modifying the cell or the population of cells to be hypoimmunogenic.
245. The method according to embodiment 244, wherein the modifying comprises reducing expression of B2M, CIITA and TCRalpha and increasing expression of CD47.
246. The method according to any preceding embodiment, wherein the cell or the population of cells comprise one or more modifications.
247. The method according to any preceding embodiment, wherein the cell or the population of cells comprise one or more modifications that (i) reduce expression of one or more MHC class I molecules and/or one or more MHC class II molecules, and/or (ii) increase expression of one or more tolerogenic factors, wherein the reduced expression of (i) and the increased expression of (ii) is relative to a cell of the same cell type that does not comprise the modifications.
248. The method according to embodiment 247, wherein the one or more modifications in (i) reduce expression of:
   a. one or more MHC class I molecules
   b. one or more MHC class II molecules; or
   c. one or more MHC class I molecules and one or more MHC class II molecules.
249. The method according to embodiment 247 or embodiment 248, wherein the one or more modifications in (i) reduce expression of one or more molecules selected from the group consisting of B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, and any combination thereof.
250. The method according to embodiment 249, wherein the cell or the population of cells do not express one or more molecules selected from the group consisting of B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, and combinations thereof.
251. The method according to any one of embodiments 247-250, wherein the one or more modifications that increase expression comprise increased cell surface expression, and/or the one or more modifications that reduce expression comprise reduced cell surface expression.
252. The method according to any one of embodiments 247-251, wherein the one or more modifications in (i) reduce expression of one or more MHC class I molecules.
253. The method according to any one of embodiments 247-252, wherein the one or more modifications in (i) reduce expression of B2M.
254. The method according to any one of embodiments 247-253, wherein the one or more modifications in (i) reduce expression of HLA-A, HLA-B, and/or HLA-C,
255. The method according to any one of embodiments 247-254, wherein the one or more modifications in (i) reduce expression of one or more MHC class II molecules.
256. The method according to any one of embodiments 247-255, wherein the one or more modifications in (i) reduce expression of CIITA.
257. The method according to any one of embodiments 247-256, wherein the one or more modifications in (i) reduce expression of HLA-DM, HLA-DO, HLA-DP, HLA-DQ, HLA-DR, RFX5, RFXANK, and/or RFXAP.
258. The method according to any one of embodiments 247-257, wherein the one or more tolerogenic factors comprise one or more tolerogenic factors selected from the group consisting of A20/TNFAIP3, C1-Inhibitor, CCL21, CCL22, CD16, CD16 Fc receptor, CD24, CD27, CD35, CD39, CD46, CD47, CD52, CD55, CD59, CD200, CR1, CTLA4-Ig, DUX4, FasL. H2-M3, HLA-C, HLA-E, HLA-E heavy chain, HLA-F, HLA-G, IDO1, IL-10, IL15-RF, IL-35, MANF, Mfge8, PD-L1, Serpinb9, and any combination thereof.
259. The method according to any one of embodiments 247-258, wherein the one or more tolerogenic factors comprise CD47.
260. The method according to any one of embodiments 247-259, wherein the one or more tolerogenic factors comprise CCL22.
261. The method according to any one of embodiments 247-260, wherein the one or more tolerogenic factors comprise CD16 or CD16 Fc receptor.
262. The method according to any one of embodiments 247-261, wherein the one or more tolerogenic factors comprise CD24.
263. The method according to any one of embodiments 247-262, wherein the one or more tolerogenic factors comprise CD39.
264. The method according to any one of embodiments 247-263, wherein the one or more tolerogenic factors comprise CR1.
265. The method according to any one of embodiments 247-264, wherein the one or more tolerogenic factors comprise CD52.
266. The method according to any one of embodiments 247-265, wherein the one or more tolerogenic factors comprise CD55.
267. The method according to any one of embodiments 247-266, wherein the one or more tolerogenic factors comprise CD200.
268. The method according to any one of embodiments 247-267, wherein the one or more tolerogenic factors comprise DUX4.
269. The method according to any one of embodiments 247-268, wherein the one or more tolerogenic factors comprise HLA-E.
270. The method according to any one of embodiments 247-269, wherein the one or more tolerogenic factors comprise HLA-G.
271. The method according to any one of embodiments 247-270, wherein the one or more tolerogenic factors comprise IDO1.
272. The method according to any one of embodiments 247-271, wherein the one or more tolerogenic factors comprise IL15-RF.
273. The method according to any one of embodiments 247-272, wherein the one or more tolerogenic factors comprise IL35.
274. The method according to any one of embodiments 247-273, wherein the one or more tolerogenic factors comprise PD-L1.
275. The method according to any one of embodiments 247-274, wherein the one or more tolerogenic factors comprise MANF.
276. The method according to any one of embodiments 247-275, wherein the one or more tolerogenic factors comprise A20/TNAIP3.
277. The method according to any one of embodiments 247-276, wherein the one or more tolerogenic factors comprise HLA-E and CD47.
278. The method according to any one of embodiments 247-277, wherein the one or more tolerogenic factors comprise two or more tolerogenic factors selected from the group consisting of CD47, CD46, and CD59, optionally wherein the one or more tolerogenic factors comprise CD47, CD46, and CD59.
279. The method according to any one of embodiments 247-278, wherein the one or more tolerogenic factors comprise two or more tolerogenic factors selected from the group consisting of CD47 and CD39, optionally wherein the one or more tolerogenic factors comprise CD47 and CD39.
280. The method according to any one of embodiments 247-279, wherein the one or more tolerogenic factors comprise two or more tolerogenic factors selected from the group consisting of CD47 and CCL22, optionally wherein the one or more tolerogenic factors comprise CD47 and CCL22.
281. The method according to any one of embodiments 247-280, wherein the one or more tolerogenic factors comprise two or more tolerogenic factors selected from the group consisting of CD47, HLA-G and PD-L1, optionally wherein the one or more tolerogenic factors comprise CD47 and PD-L1, and optionally wherein the one or more tolerogenic factors comprise CD47, HLA-G and PD-L1.
282. The method according to any one of embodiments 247-281, wherein the one or more tolerogenic factors comprise two or more tolerogenic factors selected from the group consisting of CD24, CD47, and PD-L1, optionally wherein the one or more tolerogenic factors comprise CD24, CD47, and PD-L1.
283. The method according to any one of embodiments 247-282, wherein the one or more tolerogenic factors comprise two or more tolerogenic factors selected from the group consisting of HLA-E, CD24, CD47, and PD-L1, optionally wherein the one or more tolerogenic factors comprise HLA-E, CD24, CD47, and PD-L1.
284. The method according to any one of embodiments 247-283, wherein the one or more tolerogenic factors comprise two or more tolerogenic factors selected from the group consisting of CD46, CD55, CD59, and CR1, optionally wherein the one or more tolerogenic factors comprise CD46, CD55, CD59, and CR1.
285. The method according to any one of embodiments 247-284, wherein the one or more tolerogenic factors comprise two or more tolerogenic factors selected from the group consisting of HLA-E, CD46, CD55, CD59, and CR1, optionally wherein the one or more tolerogenic factors comprise HLA-E, CD46, CD55, CD59, and CR1.
286. The method according to any one of embodiments 247-285, wherein the one or more tolerogenic factors comprise two or more tolerogenic factors selected from the group consisting of HLA-E, CD24, CD47, PD-L1, CD46, CD55, CD59, and CR1, optionally wherein the one or more tolerogenic factors comprise HLA-E, CD24, CD47, PD-L1, CD46, CD55, CD59, and CR1.
287. The method according to any one of embodiments 247-286, wherein the one or more tolerogenic factors comprise HLA-E and PD-L1.
288. The method according to any one of embodiments 247-287, wherein the one or more tolerogenic factors comprise two or more tolerogenic factors selected from the group consisting of HLA-E, PD-L1, and A20/TNFAIP, optionally wherein the one or more tolerogenic factors comprise HLA-E, PD-L1, and A20/TNFAIP.
289. The method according to any one of embodiments 247-288, wherein the one or more tolerogenic factors comprise two or more tolerogenic factors selected from the group consisting of HLA-E, PD-L1, and MANF, optionally wherein the one or more tolerogenic factors comprise HLA-E, PD-L1, and MANF.
290. The method according to any one of embodiments 247-289, wherein the one or more tolerogenic factors comprise two or more tolerogenic factors selected from the group consisting of HLA-E, PD-L1, A20/TNFAIP, and MANF, optionally wherein the one or more tolerogenic factors comprise HLA-E, PD-L1, A20TNFAIP, and MANF.
291. The method according to embodiment 246, wherein the cell or the population of cells comprise one or more modifications that (i) reduce expression of one or more MHC class I molecules and one or more MHC class II molecules, and (ii) increase expression of
CD47, wherein the reduced expression of (i) and the increased expression of(ii) is relative to a cell of the same cell type that does not comprise the modifications.
292. The method according to embodiment 291, wherein the one or more modifications in (i) reduce expression of one or more molecules selected from the group consisting of B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, and any combination thereof.
293. The method according to embodiment 291 or 292, wherein the one or more modifications in (i) reduce expression of B2M.
294. The method according to any one of embodiments 291-293, wherein the one or more modifications in (i) reduce expression of HLA-A, HLA-B, and/or HLA-C,
295. The method according to any one of embodiments 291-294, wherein the one or more modifications in (i) reduce expression of CIITA.
296. The method according to any one of embodiments 291-294, wherein the one or more modifications in (i) reduce expression of HLA-DP, HLA-DR, and/or HLA-DQ,
297. The method according to any one of embodiments 246-296, wherein the cell or the population of cells further comprise one or more modifications that increase expression of one or more additional tolerogenic factors.
298. The method according to embodiment 297, wherein the one or more additional tolerogenic factors comprise one or more tolerogenic factors selected from the group consisting of A20/TNFAIP3, C1-Inhibitor, CCL21, CCL22, CD16, CD16 Fc receptor, CD24, CD27, CD35, CD39, CD46, CD47, CD52, CD55, CD59, CD200, CR1, CTLA4-Ig, DUX4, FasL, H2-M3, HLA-C, HLA-E, HLA-E heavy chain, HLA-F, HLA-G, IDO1, IL-10, IL15-RF, IL-35, MANF, Mfge8, PD-L1, Serpinb9, and any combination thereof.
299. The method according to embodiment 298, wherein the one or more additional tolerogenic factors comprise CD47.
300. The method according to any one of embodiments 246-299, wherein the cell or the population of cells further comprise one or more modifications that reduce expression of one or more additional molecules.
301. The method according to embodiment 300, wherein the one or more additional molecules comprises B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, ABO, CADM1, CD58, CD38, CD142, CD155, CEACAM1, CTLA-4, FUT1, ICAM1, IRF1, MIC-A, MIC-B, NLGN4Y, PCDH11Y, PD-1, a protein that is involved in oxidative or ER stress, RHD, TRAC, TRB, optionally wherein the protein that is involved in oxidative or ER stress is selected from the group consisting of TXNIP, PERK, IRE1α, and DJ-1 (PARK7).
302. The method according to embodiment 300 or embodiment 301, wherein the one or more additional molecules comprise one or more Y chromosome proteins, optionally Protocadherin-11 Y-linked (PCDH11Y) and/or Neuroligin-4 Y-linked (NLGN4Y).
303. The method according to any one of embodiments 300-302, wherein the one or more additional molecules comprise one or more NK cell ligands, optionally MIC-A and/or MIC-B.
304. The method according to any one of embodiments 300-303, wherein the one or more additional molecules comprise one or more proteins involved in oxidative or ER stress, optionally thioredoxin-interacting protein (TXNIP), PKR-like ER kinase (PERK), inositol-requiring enzyme 1α (IRE1α), and/or DJ-1 (PARK7).
305. The method according to any one of embodiments 300-304, wherein the one or more additional molecules comprise one or more blood antigen proteins, optionally ABO, FUT1 and/or RHD.
306. The method according to any one of embodiments 246-305, wherein the cell or the population of cells further comprise one or more modifications that reduce expression of B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, ABO, CADM1, CD58, CD38, CD142, CD155, CEACAM1, CTLA-4, FUT1, ICAM1, IRF1, MIC-A, MIC-B, NLGN4Y, PCDH11 Y, PD-1, a protein that is involved in oxidative or ER stress, RHD, TRAC, TRB, optionally wherein the protein that is involved in oxidative or ER stress is selected from the group consisting of TXNIP, PERK, IRE1α, and DJ-1 (PARK7).
307. The method according to embodiment 306, wherein TRB is TRBC1, TRBC2, or TRBC1 and TRBC2.
308. The method according to any one of embodiments 246-307, wherein reduced expression comprises no cell surface expression or no detectable cell surface expression.
309. The method according to any one of embodiments 246-308, wherein reduced expression comprises reduced mRNA expression, optionally wherein reduced expression comprises no detectable mRNA expression.
310. The method according to any one of embodiments 246-310, wherein reduced expression comprises reduced protein expression or reduced protein activity, optionally wherein reduced expression comprises no detectable protein expression or protein activity.
311. The method according to any one of embodiments 246-310, wherein reduced expression comprises eliminating activity of a gene encoding or regulating the expression of i) the one or more MHC class I molecules and/or the one or more MHC class II molecules, or ii) the one or more additional molecules.
312. The method according to any one of embodiments 246-311, wherein reduced expression comprises inactivation or disruption of an allele of a gene encoding or regulating the expression of i) the one or more MHC class I molecules and/or the one or more MHC class II molecules, or ii) the one or more additional molecules.
313. The method according to any one of embodiments 246-312, wherein reduced expression comprises inactivation or disruption of both alleles of a gene encoding or regulating the expression of i) the one or more MHC class I molecules and/or the one or more MHC class II molecules, or ii) the one or more additional molecules.
314. The method according to any one of embodiments 246-313, wherein the one or more modifications to reduce expression comprises an indel in a gene encoding or regulating the expression of i) the one or more MHC class I molecules and/or the one or more MHC class II molecules, or ii) the one or more additional molecules.
315. The method according to any one of embodiments 246-314, wherein the one or more modifications to reduce expression comprises a frameshift mutation or a deletion of a contiguous stretch of genomic DNA of a gene encoding or regulating the expression of i) the one or more MHC class I molecules and/or the one or more MHC class II molecules, or ii) the one or more additional molecules.
316. The method according to any one of embodiments 246-315, wherein the one or more modifications to reduce expression comprises inactivation or disruption of all coding sequences of a gene encoding or regulating the expression of i) the one or more MHC class I molecules and/or the one or more MHC class II molecules, or ii) the one or more additional molecules.
317. The method according to any one of embodiments 246-316, wherein the one or more modifications to reduce expression comprises knocking out a gene encoding or regulating the expression of i) the one or more MHC class I molecules and/or the one or more MHC class II molecules, or ii) the one or more additional molecules.
318. The method according to any one of embodiments 246-317, wherein the cell or the population of cells comprise one or more modifications that:
   a. reduce expression of MHC class I and/or MHC class II molecules;
   b. increase expression of CD47; and
   c. increase expression of CCL22.
319. The method according to any one of embodiments 246-317, wherein the cell or the population of cells comprise one or more modifications that:
   a. reduce expression of MHC class I and/or MHC class II molecules;
   b. increase expression of CD47; and
   c. increase expression of CD39.
320. The method according to any one of embodiments 246-317, wherein the cell or the population of cells comprise one or more modifications that:
   a. reduce expression of MHC class I and/or MHC class II molecules;
   b. increase expression of CD47; and
   c. increase expression of CD46 and CD59.
321. The method according to any one of embodiments 246-317, wherein the cell or the population of cells comprise one or more modifications that:
   a. reduce expression of MHC class I and/or MHC class II molecules;
   b. increase expression of CD47; and
   c. increase expression of PD-L1.
322. The method according to any one of embodiments 246-317, wherein the cell or the population of cells comprise one or more modifications that:
   a. reduce expression of MHC class I and/or MHC class II molecules;
   b. increase expression of CD47; and
   c. increase expression of HLA-G and PD-L1.
323. The method according to any one of embodiments 246-317, wherein the cell or the population of cells comprise one or more modifications that:
   a. reduce expression of MHC class I and/or MHC class II molecules;
   b. increase expression of CD47; and
   c. reduced expression of CD142 (TF).
324. The method according to any one of embodiments 246-317, wherein the cell or the population of cells comprise one or more modifications that:
   a. reduce expression of MHC class I and/or MHC class I1 molecules;
   b. increase expression of CD47; and
   c. reduced expression of MIC-A and/or MIC-B.
325. The method according to any one of embodiments 246-317, wherein the cell or the population of cells comprise one or more modifications that:
   a. reduce expression of MHC class I and/or MHC class II molecules; and
   b. increase expression of CD24.
326. The method according to any one of embodiments 246-317, wherein the cell or the population of cells comprise one or more modifications that:
   a. reduce expression of MHC class I and/or MHC class II molecules; and
   b. increase expression of CD200.
327. The method according to any one of embodiments 246-317, wherein the cell or the population of cells comprise one or more modifications that:
   a. reduce expression of MHC class I and/or MHC class II molecules; and
   b. increase expression of CD52.
328. The method according to any one of embodiments 246-317, wherein the cell or the population of cells comprise one or more modifications that:
   a. reduce expression of MHC class I and/or MHC class II molecules; and
   b. increase expression of DUX4.
329. The method according to any one of embodiments 246-317, wherein the cell or the population of cells comprise one or more modifications that:
   a. reduce expression of MHC class I and/or MHC class II molecules; and
   b. increase expression of IDO1.
330. The method according to any one of embodiments 246-317, wherein the cell or the population of cells comprise one or more modifications that:
   a. reduce expression of MHC class I and/or MHC class II molecules; and
   b. increase expression of IL-35.
331. The method according to any one of embodiments 246-317, wherein the cell or the population of cells comprise one or more modifications that:
   a. reduce expression of MHC class I and/or MHC class II molecules; and
   b. increase expression of PD-L1.
332. The method according to any one of embodiments 246-317, wherein the cell or the population of cells comprise one or more modifications that:
   a. reduce expression of MHC class I and/or MHC class II molecules; and
   b. increase expression of HLA-E.
333. The method according to any one of embodiments 246-317, wherein the cell or the population of cells comprise one or more modifications that:
   a. reduce expression of MHC class I and/or MHC class II molecules; and
   b. increase expression of HLA-G.
334. The method according to any one of embodiments 246-317, wherein the cell or the population of cells comprise one or more modifications that:
   a. reduce expression of MHC class I and/or MHC class II molecules;
   b. reduce expression of CD155; and
   c. increase expression of HLA-E.
335. The method according to any one of embodiments 246-317, wherein the cell or the population of cells comprise one or more modifications that:
   a. reduce expression of MHC class I molecules;
   b. reduce expression of RFXANK;
   c. increase expression of HLA-E.
336. The method according to any one of embodiments 246-317, wherein the cell or the population of cells comprise one or more modifications that:
   a. reduce expression of MHC class I and/or MHC class II molecules;
   b. reduce expression of MIC-A and/or MIC-B;
   c. increase expression of one or more of CD47, CD24 and PD-L1; and
   d. increase expression of CD46. CD55, CD59 and CR1.
337. The method according to any one of embodiments 246-317, wherein the cell or the population of cells comprise one or more modifications that:
   a. reduce expression of MHC class I molecules;
   b. reduce expression of MIC-A and/or MIC-B;
   c. reduce expression of TXNIP; and
   d. increase expression of PD-L1 and HLA-E.
338. The method according to embodiment 337, wherein the modifications further increase expression of A20/TNFAIP3 and MANF.
339. The method according to any one of embodiments 246-338, wherein the one or more modifications that reduce expression of MHC class I and/or MHC class II molecules consist of one or more modifications that reduce expression of MHC class I molecules.
340. The method according to any one of embodiments 246-338, wherein the one or more modifications that reduce expression of MHC class I and/or MHC class II molecules consist of one or more modifications that reduce expression of MHC class II molecules.
341. The method according to any one of embodiments 246-338, wherein the one or more modifications that reduce expression of MHC class I and/or MHC class II molecules consist of one or more modifications that reduce expression of MHC class I molecules and MHC class II molecules.
342. The method according to any one of embodiments 246-341, wherein increased expression comprises increased mRNA expression.
343. The method according to any one of embodiments 246-342, wherein increased expression comprises increased protein expression or protein activity.
344. The method according to any one of embodiments 246-343, wherein increased expression comprises increasing activity of a gene encoding or regulating the expression of i) the one or more tolerogenic factors, or ii) the one or more additional tolerogenic factors.
345. The method according to embodiment 344, wherein the gene is an endogenous gene and the one or more modifications comprise one or more modifications of an endogenous promoter.
346. The method according to embodiment 344, wherein the gene is an endogenous gene and the one or more modifications comprise introduction of a heterologous promoter.
347. The method according to embodiment 346, wherein the heterologous promoter is selected from the group consisting of a CAG promoter, cytomegalovirus (CMV) promoter, EF1α promoter, EF1α short promoter, PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein Barr virus (EBV) promoter, and Rous sarcoma virus (RSV) promoter, and UBC promoter.
348. The method according to any one of embodiments 246-347, wherein the cell or the population of cells comprise one or more transgenes.
349. The method according to embodiment 348, wherein the one or more transgenes encode at least one of the one or more tolerogenic factors or the one or more additional tolerogenic factors.
350. The method according to embodiment 348 or embodiment 349, wherein the one or more transgenes encode at least one of the one or more additional tolerogenic factors.
351. The method according to any one of embodiments 348-350, wherein the one or more transgenes encode one or more additional molecules.
352. The method according to any one of embodiments 348-351, wherein the one or more transgenes comprise one or more regulatory elements.
353. The method according to any one of embodiments 348-352, wherein the one or more transgenes are operably linked to the one or more regulatory elements.
354. The method according to embodiment 352 or embodiment 353, wherein the one or more regulatory elements comprise one or more promoters, enhancers, introns, terminators, translation initiation signals, polyadenylation signals, replication elements, RNA processing and export elements, transposons, transposases, insulators, internal ribosome entry sites (IRES), 5′UTRs, 3′UTRs, mRNA 3′ end processing sequences, boundary elements, locus control regions (LCR), matrix attachment regions (MAR), recombination or cassette exchange sequences, linker sequences, secretion signals, resistance markers, anchoring peptides, localization signals, fusion tags, affinity tags, chaperonins, and proteases.
355. The method according to embodiment 354, wherein the promoter is selected from the group consisting of a CAG promoter, cytomegalovirus (CMV) promoter, EF1α promoter, EF1α short promoter, PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein Barr virus (EBV) promoter, and Rous sarcoma virus (RSV) promoter, and UBC promoter.
356. The method according to any one of embodiments 348-355, wherein the cell or the population of cells comprise one or more vectors encoding the one or more transgenes.
357. The method according to embodiment 356, wherein at least one of the one or more vectors is a multicistronic vector.
358. The method according to embodiment 357, wherein the multicistronic vector encodes at least one of the one or more tolerogenic factors or the one or more additional tolerogenic factors.
359. The method according to embodiment 357 or embodiment 358, wherein the multicistronic vector further encodes at least one of the one or more tolerogenic factors or the one or more additional tolerogenic factors.
360. The method according to any one of embodiments 357-359, wherein the multicistronic vector further encodes at least one of the one or more additional molecules.
361. The method according to any one of embodiments 348-360, wherein the one or more transgenes are separated by one or more linker sequences.
362. The method according to embodiment 361, wherein the one or more linker sequences comprise an IRES sequence or a cleavable peptide sequence.
363. The method according to embodiment 362, wherein the cleavable peptide sequence comprises a self-cleavable peptide, optionally a 2A peptide.
364. The method according to embodiment 363, wherein the 2A peptide is selected from the group consisting of a F2A sequence, an E2A sequence, a P2A sequence, and a T2A sequence.
365. The method according to any one of embodiments 362-364, wherein the cleavable peptide sequence comprises a protease cleavable sequence or a chemically cleavable sequence.
366. The method according to any one of embodiments 358-365, wherein the one or more tolerogenic factors, the one or more additional tolerogenic factors, and/or the one or more additional molecules are operably linked to the same promoter.
367. The method according to embodiment 366, wherein the promoter is a constitutive promoter.
368. The method according to embodiment 366 or embodiment 367, wherein the promoter is selected from the group consisting of a CAG promoter, cytomegalovirus (CMV) promoter, EF1α promoter, EF1α short promoter, PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein Barr virus (EBV) promoter, and Rous sarcoma virus (RSV) promoter, and UBC promoter.
369. The method according to any one of embodiments 351-368, wherein the one or more additional molecules comprise a chimeric antigen receptor (CAR).
370. The method according to any preceding embodiment, wherein the cell or the population of cells comprise a chimeric antigen receptor (CAR).
371. The method according to any preceding embodiment, wherein the evaluating predicted cell function is performed on the cells following introduction of a CAR transgene modifications.
372. The method according to any preceding embodiment, wherein the at least one cell parameter comprises determining the presence of a CAR transgene modification in the cell or the population of cells.
373. The method according to any preceding embodiment, wherein the at least one cell parameter comprises determining CAR expression in the cell or the population of cells.
374. The method according to any one of embodiments 369-373, wherein the CAR comprises a signal peptide, an extracellular binding domain specific to CD19, a hinge domain, a transmembrane domain, an intracellular costimulatory domain, and/or an intracellular signaling domain.
375. The method according to any one of embodiments 369-374, wherein the CAR comprises a CD5-specific CAR, a CD19-specific CAR, a CD20-specific CAR, a CD22-specific CAR, a CD23-specific CAR, a CD30-specific CAR, a CD33-specific CAR, CD38-specific CAR, a CD70-specific CAR, a CD123-specific CAR, a CD138-specific CAR, a Kappa, Lambda, B cell maturation agent (BCMA)-specific CAR, a G-protein coupled receptor family C group 5 member D (GPRC5D)-specific CAR, a CD123-specific CAR, a LeY-specific CAR, a NKG2D ligand-specific CAR, a WT1-specific CAR, a GD2-specific CAR, a HER2-specific CAR, a EGFR-specific CAR, a EGFRvIII-specific CAR, a B7H3-specific CAR, a PSMA-specific CAR, a PSCA-specific CAR, a CAIX-specific CAR, a CD171-specific CAR, a CEA-specific CAR, a CSPG4-specific CAR, a EPHA2-specific CAR, a FAP-specific CAR, a FRa-specific CAR, a IL-13Ra-specific CAR, a Mesothelin-specific CAR, a MUC1-specific CAR, a MUC16-specific CAR, a ROR1-specific CAR, a C-Met-specific CAR, a CD133-specific CAR, a Ep-CAM-specific CAR, a GPC3-specific CAR, a HPV16-E6-specific CAR, a IL13Ra2-specific CAR, a MAGEA3-specific CAR, a MAGEA4-specific CAR, a MART1-specific CAR, a NY-ESO-1-specific CAR, a VEGFR2-specific CAR, a α-Folate receptor-specific CAR, a CD24-specific CAR, a CD44v7/8-specific CAR, a EGP-2-specific CAR, a EGP-40-specific CAR, a erb-B2-specific CAR, a erb-B 2,3,4-specific CAR, a FBP-specific CAR, a Fetal acethylcholine e receptor-specific CAR, a Gm-specific CAR, a G3-specific CAR, a HMW-MAA-specific CAR, a IL-11Rα-specific CAR, a KDR-specific CAR, a Lewis Y-specific CAR, a L1-cell adhesion molecule-specific CAR, a MAGE-A1-specific CAR, a Oncofetal antigen (h5T4)-specific CAR, a TAG-72-specific CAR, or a CD19/CD22-bispecific CAR.
376. The method according to any one of embodiments 369-375, wherein the CAR comprises a CD19-specific CAR, a CD20-specific CAR, a CD22-specific CAR, a CD38-specific CAR, a CD123-specific CAR, a CD138-specific CAR, a BCMA-specific CAR, or a CD19/CD22-bispecific CAR.
377. The method according to any one of embodiments 369-376, wherein the CAR is specific for CD19.
378. The method according to any one of embodiments 369-377, wherein the CAR is specific for CD22.
379. The method according to any one of embodiments 369-378, wherein the CAR is a CD19/CD22-bispecific CAR.
380. The method according to any preceding embodiment, wherein the cell or the population of cells are CAR-T cells.
381. The method according to embodiment 380, wherein the method further comprises determining expression of the CAR in the cells.
382. The method according to any one of embodiments 351-381, wherein the one or more additional molecules comprise one or more safety switches.
383. The method according to embodiment 382, wherein the method further comprises determining the presence of one or more safety switches in the cell or the population of cells.
384. The method according to any preceding embodiment, wherein the at least one cell parameter comprises assaying safety switch activity in the cell or the population of cells.
385. The method according to any preceding embodiment, wherein the evaluating predicted cell function is performed on the cells following introduction of safety switch modifications.
386. The method according to any one of embodiments 382-385, wherein the one or more safety switches are capable of controlled killing of the cell or the population of cells.
387. The method according to any one of embodiments 382-386, wherein the one or more safety switches induce controlled cell death in the presence of a drug or prodrug, or upon activation by a selective exogenous compound.
388. The method according to any one of embodiments 382-387, wherein the one or more safety switches comprise is an inducible protein capable of inducing apoptosis of the cell or the population of cells.
389. The method according to embodiment 388, wherein the inducible protein capable of inducing apoptosis of the cell or the population of cells is a caspase protein.
390. The method according to embodiment 389, wherein the caspase protein is caspase 9.
391. The method according to any one of embodiments 382-390, wherein the one or more safety switches comprise one or more suicide genes.
392. The method according to embodiment 391, wherein the one or more suicide genes are selected from the group consisting of cytosine deaminase (CyD), herpesvirus thymidine kinase (HSV-Tk), an inducible caspase 9 (iCaspase9), and rapamycin-activated caspase 9 (rapaCasp9).
393. The method according to any one of embodiments 382-392, wherein the safety switch is an “uncloaking” system wherein upon activation, cells downregulate expression of immunosuppressive factors and/or upregulate expression of immune signaling molecules thereby marking the cell for elimination by the host immune system.
394. The method according to any one of embodiments 348-393, wherein at least one of the one or more transgenes are integrated into the genome of the cell or the population of cells.
395. The method according to any preceding embodiment, wherein one or more transgenes are integrated into the genome of the cell or the population of cells.
396. The method according to any preceding embodiment, wherein the method further comprises determining integration of at least one of the one or more transgenes into the genome of the cell or the population of cells.
397. The method according to any preceding embodiment, wherein the evaluating predicted cell function is performed following introduction of one or more transgenes into the genome of the cell or the population of cells.
398. The method according to any preceding embodiment, wherein the at least one cell parameter comprises determining transgene expression in the cell or the population of cells.
399. The method according to embodiment 394-398, wherein integration is by non-targeted insertion into the genome of the cell or the population of cells.
400. The method according to embodiment 394-398, wherein integration is by non-targeted insertion into the genome of the cell or the population of cells using a lentiviral vector.
401. The method according to embodiment 394-398, wherein integration is by targeted insertion into a target genomic locus of the cell or the population of cells.
402. The method according to embodiment 401, wherein targeted insertion is with homology-directed repair.
403. The method according to embodiment 401 or embodiment 402, wherein the target genomic locus is selected from the group consisting of an albumin gene locus, an ABO gene locus, a B2M gene locus, a CIITA gene locus, a CCR5 gene locus, a CD142 gene locus, a CLYBL gene locus, a CXCR4 gene locus, an F3 gene locus, a FUT1 gene locus, an HMGB1 gene locus, a KDM5D gene locus, an LRP1 gene locus, a MIC-A gene locus, a MIC-B gene locus, a PPP1R12C (also known as AAVS1) gene locus, an RHD gene locus, a ROSA26 gene locus, a safe harbor gene locus, a SHS231 locus, a TAP1 gene locus, a TRAC gene locus, and a TRBC gene locus.
404. The method according to any preceding embodiment, wherein the genome of the cell or the population of cells comprises one or more gene edits in one or more genes encoding the one or more molecules of any of embodiments 196-368 having reduced expression.
405. The method according to any preceding embodiment, wherein the cell or the population of cells comprises a genome editing complex.
406. The method according to embodiment 405, wherein the genome editing complex comprises a genome targeting entity and a genome modifying entity.
407. The method according to embodiment 406, wherein the genome targeting entity localizes the genome editing complex to the target locus, optionally wherein the genome targeting entity is a nucleic acid-guided targeting entity.
408. The method according to embodiment 406 or embodiment 407, wherein the genome targeting entity is selected from the group consisting of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZF) nucleic acid binding entity, a transcription activator-like effector (TALE) nucleic acid binding entity, a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, or a functional portion thereof.
409. The method according to any one of embodiments 406-408, wherein the genome targeting entity is selected from the group consisting of Cas1, Cas2, Cas3, Cas4, Cas5, Cas6. Cas7, Cas8a. Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, dCas13b, or a functional portion thereof.
410. The method according to embodiment 406, wherein the genome modifying entity cleaves, deaminates, nicks, polymerizes, interrogates, integrates, cuts, unwinds, breaks, alters, methylates, demethylates, or otherwise destabilizes the target locus.
411. The method according to embodiment 406 or embodiment 410, wherein the genome modifying entity comprises a recombinase, integrase, transposase, endonuclease, exonuclease, nickase, helicase, DNA polymerase, RNA polymerase, reverse transcriptase, deaminase, flippase, methylase, demethylase, acetylase, a nucleic acid modifying protein, an RNA modifying protein, a DNA modifying protein, an Argonaute protein, an epigenetic modifying protein, a histone modifying protein, or a functional portion thereof.
412. The method according to any one of embodiments 406, 410 or 411, wherein the genome modifying entity selected from the group consisting of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, base editing, prime editing, a Programmable Addition via Site-specific Targeting Elements (PASTE), or a functional portion thereof.
413. The method according to any one of embodiments 406 and 410-412, wherein the genome modifying entity is selected from the group consisting of Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f(C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, FokI, dCas (D10A), dCas (H840A), dCas13a, dCas13b, a base editor, a prime editor (e.g., a target-primed reverse transcription (TPRT) editor), APOBEC1, cytidine deaminase, adenosine deaminase, uracil glycosylase inhibitor (UGI), adenine base editors (ABE), cytosine base editors (CBE), reverse transcriptase, serine integrase, recombinase, transposase, polymerase, adenine-to-thymine or “ATBE” (or thymine-to-adenine or “TABE”) transversion base editor, ten-eleven translocation methylcytosine dioxygenases (TETs), TET1, TET3, TET1CD, histone acetyltransferase p300, histone methyltransferase SMYD3, histone methyltransferase PRDM9, H3K79 methyltransferase DOT1L, transcriptional repressor, or a functional portion thereof.
414. The method according to any one of embodiments 406-413, wherein the genome targeting entity and the genome modifying entity are different domains of a single polypeptide.
415. The method according to any one of embodiments 406-414, wherein the genome editing entity and genome modifying entity are two different polypeptides that are operably linked together.
416. The method according to any one of embodiments 406-414, wherein the genome editing entity and genome modifying entity are two different polypeptides that are not linked together.
417. The method according to any one of embodiments 406-414, wherein the genome editing complex comprises a guide nucleic acid having a targeting domain that is complementary to at least one target locus, optionally wherein the guide nucleic acid is a guide RNA (gRNA).
418. The method according to any one of embodiments 406-414, wherein the one or more modifications are made by the genome editing complex.
419. The method according to embodiment 418, wherein the one or more modifications made by the genome editing complex are made by a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a TnpB nuclease, a homing endonuclease, an endonuclease-deficient-Cas protein, an enzymatically inactive Cas protein, a CRISPR-associated transposase (CAST), a Type II or Type V Cas protein, base editing, prime editing, or a Programmable Addition via Site-specific Targeting Elements (PASTE).
420. The method according to embodiment 418 or embodiment 419, wherein the one or more modifications made by the genome editing complex are made by Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, a CRISPR-associated transposase, base editing, prime editing, or Programmable Addition via Site-specific Targeting Elements (PASTE).
421. The method according to any one of embodiments 418-420, wherein the modifications made by the genome editing complex are made using a guide RNA (gRNA) having a targeting domain that is complementary to at least one target site.
422. The method according to any preceding embodiment, wherein the cell or the population of cells are human cells or animal cells.
423. The method according to any preceding embodiment, wherein the animal cells are porcine cells, bovine cells, or ovine cells.
424. The method according to any one of embodiments 1-423, wherein the cell or the population of cells are human cells.
425. The method according to any preceding embodiment, wherein the cell or the population of cells are stem cells or progenitor cells.
426. The method according to any one of embodiments 1-424, wherein the cell or the population of cells are differentiated cells derived from the stem cells or the progenitor cells.
427. The method according to embodiment 425, wherein the cell or the population of cells are progenitor cells.
428. The method according to any preceding embodiment, wherein the stem cell or progenitor cell is selected from the group consisting of an induced pluripotent stem cell, an embryonic stem cell, a hematopoietic stem cell, a mesenchymal stem cell, an endothelial stem cell, an epithelial stem cell, an adipose stem cell, a germline stem cell, a lung stem cell, a cord blood stem cell, a pluripotent stem cell (PSC), and a multipotent stem cell.
429. The method according to any one of embodiments 1-424, wherein the cell or the population of cells are differentiated cells derived from pluripotent stem cells or progenies thereof.
430. The method according to any one of embodiments 1-424, wherein the cell or the population of cells are pluripotent stem cells.
431. The method according to any one of embodiments 1-424, wherein the cell or the population of cells are hematopoietic stem cells (HSCs).
432. The method according to any one of embodiments 1-424, wherein the cell or the population of cells are multipotent cells.
433. The method according to any one of embodiments 1-424, wherein the pluripotent stem cell is an induced pluripotent stem cell.
434. The method according to any one of embodiments 1-424, wherein the cell or the population of cells are differentiated cells derived from induced pluripotent stem cells.
435. The method according to any one of embodiments 1-343, wherein the cell or the population of cells are differentiated cells derived from embryonic stem cells.
436. The method according to any preceding embodiment, wherein the evaluating predicted cell function is performed on the cells pre-differentiation.
437. The method according to any preceding embodiment wherein the cells are stem cells and the evaluating predicted cell function is performed on the cells following differentiation.
438. The method according to any preceding embodiment, wherein the evaluating predicted cell function is performed before cell differentiation, after cell differentiation, or both before and after cell differentiation.
439. The method according to any preceding embodiment, wherein the evaluating predicted cell function is performed on the cell or the population of cells prior to any differentiation.
440. The method according to any preceding embodiment, wherein the cell or the population of cells is cryopreserved prior to any differentiation and the evaluating predicted cell function is performed:
   i) before the cell or the population of cells is cryopreserved, and/or
   ii) after the cell or the population of cells has been cryopreserved and thawed.
441. The method according to any preceding embodiment, wherein the cell or the population of cells are stored prior to any differentiation and evaluating predicted cell function is performed:
   i) before the cell or the population of cells is stored, and/or
   ii) after the cell or the population of cells has been stored.
442. The method according to any preceding embodiment, wherein the evaluating predicted cell function is performed after the cell or the population of cells have been differentiated.
443. The method according to any preceding embodiment, wherein the cell or the population of cells is cryopreserved after differentiation and the evaluating predicted cell function is performed:
   i) before the cell or the population of cells is cryopreserved, and/or
   ii) after the cell or the population of cells has been cryopreserved and thawed.
444. The method according to any preceding embodiment, wherein the cell or the population of cells are stored after differentiation and evaluating predicted cell function is performed:
   i) before the cell or the population of cells is stored, and/or
   ii) after the cell or the population of cells has been stored.
445. the method according to any preceding embodiment, wherein the evaluating predicted cell function is performed after the cell or the population of cells have completed differentiation.
446. The method according to any preceding embodiment, wherein the cell or the population of cells are cryopreserved after completed differentiation and the evaluating predicted cell function is performed:
   i) before the cell or the population of cells is cryopreserved, and/or
   ii) after the cell or the population of cells has been cryopreserved and thawed.
447. The method according to any preceding embodiment, wherein the cell or the population of cells are stored after completed differentiation and evaluating predicted cell function is performed:
   i) before the cell or the population of cells is stored, and/or
   ii) after the cell or the population of cells has been stored.
448. The method according to any preceding embodiment, wherein the cell or the population of cells are autologous.
449. The method according to any one of embodiments 1-447, wherein the cell or the population of cells are allogeneic.
450. The method according to any preceding embodiment, wherein the cell or the population of cells are primary cells isolated from a single donor subject.
451. The method according to any one of embodiments 1-447, wherein the population of cells are primary cells isolated from more than one donor.
452. The method according to any one of embodiments 1-447, wherein the cell or the population of cells are derived from a single donor.
453. The method according to any one of embodiments 1-447, wherein the population of cells are derived from pooled donor cells obtained from more than one donor.
454. The method according to any preceding embodiment, wherein each donor subject is healthy or is not suspected of having a disease or condition at the time the donor sample is obtained from the individual donor.
455. The method according to any preceding embodiment, wherein the cell or the population of cells are selected from the group consisting of islet cells, beta islet cells, pancreatic islet cells, immune cells, B cells, T cells, natural killer (NK) cells, natural killer T (NKT) cells, macrophages, endothelial cells, muscle cells, cardiac muscle cells, smooth muscle cells, skeletal muscle cells, dopaminergic neurons, retinal pigmented epithelium cells (e.g., retinal pigmented epithelium (RPE) cells and thyroid cells), optic cells, hepatocytes, thyroid cells, skin cells, glial progenitor cells, neural cells (e.g., cerebral endothelial cells, dopaminergic neurons, glial cells, and hematopoietic stem cells (HSCS) cells), cardiac cells, stem cells, hematopoietic stem cells, induced pluripotent stem cells (iPSCs), mesenchymal stem cells (MSCs), embryonic stem cells (ESCs), pluripotent stem cells (PSCs), and blood cells.
456. The method according to any preceding embodiment, wherein the cell or the population of cells are immune cells.
457. The method according to any preceding embodiment, wherein the population of cells comprise different subtypes of cells.
458. The method according to any preceding embodiment, wherein the differentiated cells are NK cells or T cells.
459. The method according to any one of embodiments 1-433, wherein the cell or the population of cells are pluripotent stem cells.
460. The method according to any one of embodiments 1-433, wherein the at least one cell parameter is cell stability e.g., measured using a humming bird assay (PCR based), aCGH arrays, G-banding, and/or X-chromosome inactivation.
461. The method according to any one of embodiments 460, wherein the at least one cell parameter comprises genome sequencing (e.g., whole genome sequencing or targeted genome sequencing) or exome sequencing (e.g., whole exome sequencing or targeted exome sequencing).
462. The method according to embodiment 460 or 461 wherein the at least one cell parameter comprises genome sequencing or exome sequencing for screening for safety compromising mutations (e.g., ABCA10, ABCA12, ABCC9, ABL1, ABL2, ACVR1, AKAP9, AKT1. AKT2, AKT3, ALK, ANGPTL, ANKRD26, APC, AR, ARAF, AR1D1A, ARID1B, ASPH, ASXL1, ASXL2, ATM, ATR, ATRX, AURKA, AURKB, AXIN2, AXL, BABAM1, BAK1, BAP1, BARD1, BCL2, BCL2L11, BCOR, BCORL1, BCR, BIRC3, BLM, BMPR1A, BRAF, BRCA1, BRCA1&2 Sequencing, BRCA2, BRIP1, BRWD3, BTK, CALR, CARD11, CBL, CBLB, CBLC, CCND1, CCND2, CCNE1, CD19, CD274, CD74, CDH1, CDK12, CDK4, CDK6. CDK8, CDK9, CDKN1A, CDKN1C, CDKN2A, CDKN2B. CEBPA, CHD1, CHD3, CHD8, CHEK1, CHEK2, COG5, CRADD, CREBBP, CRLF2, CRX, CSFIR, CSF3R, CTCF, CTNNA1, CTNNB1, CUX1, DAXX, DDR2, DDX41, DEPDC5, DICER1, DIS3L2. DNAJB1, DNMT3A, DOCK7, DPYD, EBF1, EED, EGFR, EGLN1, EIF3E, EML4, ENPP3, EP300, EPAS1, EPCAM, EPHA3, EPHA5, EPHB2, EPHB6, EPO, EPOR, ERBB2, ERBB3, ERBB4, ERCC2, ERG, ESR1, ESR2, ETV6, EZH2, FAM175A, FAM19A2, FANCA, FANCB, FANCC, FANCD2, FANCE, FANCF, FANCG, FANCI, FANCL, FANCM, FBXW7, FGFR1, FGFR2, FGFR3, FGFR4, FH, FKBP1A, FLT1, FLT3, FLT4, FOXA1, FOXR2, FUBP1, GAB2, GALNT12, GATA1, GATA2, GATA3, GEN1, GLI1, GLI3, GLTSCR1, GLTSCR2, GNA11, GNAQ, GNAS, GPC3, GREM1, GRIN2A, GRM3, GYNPTH, H3-3A/H3F3A, H3-3B/H3F3B, H3C2/HISTIH3B, HDAC4, HDAC9, HIF1A, HNF1A, HNRNPU, HOOK3, HRAS, HSPH1, ID3, IDH, IDH1, IDH2, IGF1R, IKZF1, IL7R, JAK1, JAK2, JAK3, KCNJ8, KDM2B, KDM6A, KDR, KIF5B, KIT, KLF4, KMT2A, KMT2C, KMT2D, KRAS, KTN1, LYST, MAP2K1 (MEK1), MAP2K2 (MEK2), MAP2K4, MAP7, MAPK1, MAX, MC1R, MCL1, MDM2, MDM4, MED12, MEGF6, MEN1, MET, microsatellite instability, MIOS, MITF, MLH1, MLH3, MN1, MPL, MRE11A, MSH2, MSH6, MTAP, MTOR, MUTYH, MYB, MYC, MYCL, MYCL1, MYCN, MYD88, MYOD1, NAB2, NAT2, NBN, NF1, NF2, NKX2-1, NOTCH1, NOTCH2, NOTCH3, NPM1, NPRL2, NPRL3, NR4A3, NRAS, NRP1, NSD1, NT5C2, NTHL1, NTRK1, NTRK2, NTRK3, NUDT15, OFD1, PAK1, PALB2, PAX5, PBRM1, PDCD1LG2, PDGFRA, PDGFRB, PHF6, PHOX2B, PIGA, PIK3CA, PIK3CB, PIK3R1, PLCG2, PLK1, PLK2, PLK3, PLK4, PML, PMS2, POLD1, POLE, PPM1 D, PPP1CB, PRKAR1A, PRPF40B, PRPS1, PTCH1, PTEN, PTPN11, PTPRD, QKI, RAC1, RAD21, RAD51B, RAD51C, RAD51D, RAF1, RARA, RASA1, RB1, RECQL, RELA, RET, RHEB, RICTOR, RINT1, RIT1, ROR1, ROS1, RPL10, RPL31, RPS15, RPS20, RPTOR, RRM1, RRM2, RSPO2, RSPO3, RUNX1, SAMD9L, SDHA, SDHB, SDHC, SDHD, SETBP1, SETD2, SF1, SF3B1, SH2B3, SHH, SIGLEC10, SLC25A13, SLX4, SMAD2, SMAD3, SMAD4, SMARCA4, SMARCB1, SMC1A, SMC3, SMO, SNAPC3, SPOP, SPRY4, SRC, SRP72, SRSF2, STAG2, STAT5B, STAT6, STK11, SUFU, SUZ12, TACC3, TACSTD2, TCF3, TERC, TERT, TET1, TET2, TET3, TFE3, TFG, TGFBR2, THOR, THORplex, TLX1, TMB, TMPRSS2, total mutation burden, TP53, TP73, TRAF7, TRRAP, TSC1, TSC2, TTYH1, U2AF1, U2AF2, UBR5, USP7, VHL, WRN, WT1, XRCC2, YAP1, ZBTB16, ZFTA/C11orf95, and ZRSR2Error! Hyperink reference not valid.).
463. The method according to any one of embodiments 460-462, wherein the at least one cell parameter comprises screening for disease causing mutations in genes shown in Table 1 for cardiac cells; Table 2 for beta cells; Table 3 for T cells and Table 4 for neuronal cells.
464. The method according to any one of embodiments 460-463, wherein the at least one cell parameter comprises target cell yield after cell differentiation.
465. The method according to any one of embodiments 1-433, wherein the cell or the population of cells are T cells.
466. The method according to embodiment 465, wherein the T cells are stem cell derived T cells.
467. The method according to embodiment 465 or 466, wherein the T cells are primary T cells.
468. The method according to any one of embodiments 465-467, wherein the T cells are CAR- T cells, optionally as defined in any one of embodiments 369-381.
469. The method according to any one of embodiments 465-468, wherein the T-cells are CD3+ T cells, CD4+ T cells, CDS+ T cells, naïve T cells, regulatory T (Treg) cells, non-regulatory T cells, Th1 cells, M2 cells, Th9 cells, Th17 cells, T-follicular helper (Tfh) cells, cytotoxic T lymphocytes (CTL), effector T (Teff) cells, central memory T cells, effector memory T cells, effector memory T cells expressing CD45RA (TEMRA cells), tissue-resident memory (Trm) cells, virtual memory T cells, innate memory T cells, memory stem cell (Tse), γδ T cells, or a combination thereof.
470. The method according to any one of embodiments 465-469, wherein the T cells are cytotoxic T-cells, helper T-cells, memory T-cells, regulatory T-cells, tumor infiltrating lymphocytes, or a combination thereof.
471. The method according to any one of embodiments 465-470, wherein the subtypes of cells are T cell subtypes.
472. The method according to embodiment 471, wherein the T cell subtypes comprise at least one of the T cell subtypes selected from CD4 TN (naïve T), CD4 TSCM (stem memory), CD4 TCM (central memory), CD4 TEM (effector memory), CD4 TEFF (effector), CD4+CD27+, CD8 TN (naïve T), CD8 TSCM (stem memory), CD8 TCN (central memory), CD8 TEM (effector memory), CD8 TEFF (effector), CD8+CD27+.
473. The method according to any one of embodiments 465472, wherein the cell or the population of cells is categorised as exceptional or usable if the cell or the population of cells shows ahigh amount of mucosal-associated invariant T (MAIT) relative to a reference cell or population of cells.
474. The method according to any one of embodiments 465-473, wherein the cell or the population of cells is categorised as exceptional or usable if the cell or the population of cells shows a high amount of T Cell Receptor Beta Variable 28 (TRBV28) relative to a reference cell or population of cells.
475. The method according to any one of embodiments 465474, wherein the cell or the population of cells is categorised as exceptional or usable if the cell or the population of cells shows a high amount of Interleukin-17A (IL17A) relative to a reference cell or population of cells.
476. The method according to any one of embodiments 465-475, wherein the cell or the population of cells is categorised as exceptional or usable if the cell or the population of cells is less activated and has a reduced NK-like signature relative to a reference cell or population of cells.
477. The method according to any one of embodiments 465476, wherein the cell or the population of cells is categorised as exceptional or usable if the cell or population of cells has an activated phenotype and/or Th1/Tc1 and Th17/Tc17 states.
478. The method according to any one of embodiments 1-433, wherein the cell or the population of cells are natural killer (NK) cells.
479. The method according to embodiment 478, wherein the NK cells are stem cell derived NK cells.
480. The method according to embodiment 478, wherein the NK cells are primary NK cells.
481. The method according to embodiment 433-480, wherein the NK cells are CAR-NK cells, optionally as defined in any one of embodiments 369-381.
482. The method according to any one of embodiments 478-481, wherein the at least one cell parameter includes production of factors modulating the function of other immune cells (e.g., interferon-γ (IFN-γ), granulocyte macrophage colony-stimulating factor (GM-CSF); and/or chemokines (e.g., CCL1, CCL2, CCL3, CCL4, CCL5, and CXCL8).
483. The method according to any one of embodiments 478-482, wherein the at least one cell parameter comprises determining if the cell is CD56+ and CD16−; CD56− and CD16+; CD561o and CD16+; or CD56+ and CD3−.
484. The method according to any one of embodiments 478-483, wherein the cell or the population of cells are islet cells.
485. The method according to any one of embodiments embodiment 478-484, wherein the islet cells comprise up to 5, 10. 15, 20, 25, 35, or 40% alpha cells.
486. The method according to any one of embodiments 478-485, wherein the islet cells comprise up to 10, 15, 20, 25, 35, 40, 45, 50, 55, 60, 65, 70, 75, or 80% beta cells.
487. The method according to any one of embodiments 478-486, wherein the islet cells comprise up to 1, 2, 3, 4. 5, 6, 7, 8, 9, 10, 15, 20, or 25% delta cells.
488. The method according to any one of embodiments 478-484, wherein the cell or the population of cells are beta cells.
489. The method according to embodiment 488, wherein the beta cells are stem cell derived
490. The method according to embodiment 488, wherein the beta cells are primary beta cells.
491. The method according to any one of embodiments 484-490, wherein the at least one cell parameter comprises insulin production from beta cells (e.g., using an ELISA assay).
492. The method according to any one of embodiments 484-490, wherein the at least one cell parameter comprises Amylin or C-peptide production from beta cells.
493. The method according to any one of embodiments 1-433, wherein the cell or the population of cells are B cells.
494. The method according to embodiment 493, wherein the B cells comprise precursor or immature B cells, naïve mature B cells, memory B cells, plasmablasts, and/or plasma cells.
495. The method according to embodiment 493 or 494, wherein the B cells are CAR-B cells, optionally as defined in any one of embodiments 369-381.
496. The method according to any one of embodiments 493-495, wherein the B cells are stem cell derived B cells.
497. the method according to any one of embodiments 493-495, wherein the B cells are primary B cells.
498. The method according to any one of embodiments 493-497, wherein the at least one cell parameter comprises determining antibody production by the cell or the population of cells.
499. The method according to any one of embodiments 493-498, wherein the at least one cell parameter comprises determining:
   xvi. expression and/or secretion of certain cytokines, such as IFNγ, IL-2, IL-4, IL-6, IL-12 and TNFα;
   xvii. production and/or secretion of exogenous protein;
   xviii. expression of one or more of (such as all of) PAX5, BACH2, BCL-2. OBF1, OCT2, PU.1, SPIB, ETS1, and IRF8;
   xix. expression of one or more of (such as all of) IRF4, BLIMP1, and XBP1;
   xx. expression of one or more of (such as all of) CD19, CD20, CD21, CD22, CD23, and CD24;
   xxi. expression of one or more of (such as all of) CD10, CD27, and CD38;
   xxii. expression of one or more of (such as all of) IRF4, BLIMP1, and XBP1;
   xxiii. expression of one or more of (such as all of) PAX5, BACH2, BCL-2, OBF1, OCT2, PU.1, SPIB, ETS1, and IRF8;
   xxiv. expression of one or more of (such as all of) CD19, CD38, CD27, CD269, and MHCII;
   xxv. expression of one or more of (such as all of) CD20 and/or CD138;
   xxvi. expression of one or more of (such as all of) IRF4, BLIMP1, and XBP1;
   xxvii. expression of one or more of (such as all of) CXCR4, CD27, CD38, CD138, and CD269;
   xxviii. expression of one or more of (such as all of) CD19, CD20, and MHCII;
   xxix. expression of one or more of (such as all of) CD19, CD20, CD40, CXCR4, CXCR5, and CXCR7; and/or
   xxx. cell surface levels of CD23 and/or CD38
500. The method according to any one of embodiments 1-433, wherein the cell or the population of cells are macrophages.
501. The method according to embodiment 500, wherein the macrophages are CAR-macrophage cells, optionally as defined in any one of embodiments 369-381.
502. The method according to embodiment 500 or 501, wherein the macrophages are stem cell derived macrophages.
503. The method according to embodiment 500 or 501, wherein the macrophages are primary macrophages.
503a. The method according to any one of embodiments 500-503, wherein the at least one cell parameter comprises determining expression of M1 markers, such as HLA DR, CD86, CD80, and PDL1, and/or M2 markers, such as CD206, CD163; and/or determining targeted effector activity.
504. The method according to any one of embodiments 1-433, wherein the cell or the population of cells are hepatocytes.
505. The method according to embodiment 504, wherein the hepatocytes are stem cell derived hepatocytes.
506. The method according to embodiment 504, wherein the hepatocytes are primary hepatocytes.
507. The method according to any one of embodiments 504-506, wherein the at least one cell parameter comprises determining:
   xxvi. plateability;
xxvii. P450 induction;
   xxviii. glycogen synthesis capability and/or storage capability;
   xxix. expression of one or more urea cycle pathway enzymes (e.g., using a ureagenesis assay);
   xxx. presence or absence of a genetic aberration associated with a liver- associated monogenic disease;
   xxxi. albumin secretion;
   xxxii. exhibits α-1 antitrypsin (A1AT) secretion;
   xxxiii. coagulation Factor V secretion;
   xxxiv. lipid (e.g., VLDL, LDL, and HDL) uptake and/or storage capability;
   xxxv. indocyanine green (ICG) uptake and/or clearance capability;
   xxxvi. cytochrome p450 activity;
   xxxvii. asialoglycoprotein receptor expression (e.g., ASGR1 and/or ASGR2 expression;
   xxxviii. alpha-fetoprotein (AFP) expression;
   xxxix. gamma-glutanyl transpeptidase activity;
   xl. SOX9 expression;
   xli. keratin, type I cytoskeletal 18 (KRT18) expression;
   xlii. HNF4A (e.g., HNF4a and HNF4a) expression;
   xliii. G6PC expression;
   xliv. expression of hepatocyte markers HNF4a, ALB, CYP2C9;
   xlv. expression of the terminal differentiation marker PEPCK1;
   xlvi. expression of functional CYP enzymes, including CYP3A4, CYP2C9, CYP2B6, CYP1A2, CYP1A1, CYP2D6, CYP3A7, and CYP2E1;
   xlvii. expresses the markers HNF4a and ALB;
   xlviii. functional glucose metabolism;
   xlix. functional lipid metabolism; and/or
   l. expression of the terminal differentiation markers PEPCK1 and/or TAT.
508. The method according to any one of embodiments 1-433, wherein the cell or the population of cells are neural cells (e.g., cerebral endothelial cells; dopaminergic neurons, glial cells, and hematopoietic stem cells (HSCS) cells).
509. The method according to embodiment 508 wherein the neural cells are glial cells.
510. The method according to embodiment 508 or 509, wherein the neural cells are stem cell derived neural cells.
511. The method according to embodiment 508 or 509, wherein the neural cells are primary neural cells.
512. The method according to any one of embodiments 508-511, wherein the at least one cell parameter comprises determining:
   v. expression of one or more markers selected from NKX2.2, PAX6, SOX1 0, brain derived neurotrophic factor BDNF, neutrotrophin-3 NT-3, NT-4, epidermal growth factor EGF, ciliary neurotrophic factor CNTF, nerve growth factor NGF, FGF8, EGFR, OLIG1, OLIG2, myelin basic protein MBP, GAP-43, LNGFR, nestin, GFAP, CD11b, CD11c, CD105, CX3CR1, P2RY12, IBA-1, TMEM119, and CD45;
   vi. expression of one or more markers selected from A2B5, CD9, CD133, CD140a, FOXG1, GalC, GD3, GFAP, nestin, NG2, MBP, Musashi, 04, Olig1, Olig2, PDGFaR, S1O0P, glutamine synthetase, connexin 43, vimentin, BLBP, and GLAST;
   vii. expression of one or more markers selected from PSA-NCAM, CD9, CD11, CD32, CD36, CD105, CD140a, nestin, and PDGFaR; and/or
   viii. assaying cell morphology.
513. The method according to any one of embodiments 1-433, wherein the cell or the population of cells are cardiac cells.
514. The method according to embodiment 513, wherein the cardiac cells are cardiomyocytes.
515. The method according to embodiment 513 or 514, wherein the cardiac cells are stem cell derived cardiac cells.
516. The method according to embodiment 513 or 514, wherein the cardiac cells are primary cardiac cells.
517. The method according to any one of embodiments 513-516, wherein the at least one cell parameter comprises determining:
   ix. assaying electrophysical potential;
   x. expression of one or more markers selected from Table 1;
   xi. intrinsic beat rate below 90 beats per minute;
   xii. action potential duration between 150-300 milliseconds;
   xiii. ratios of expression levels of MYL2:MYL7, MYH7;MYH6, and TNNI3.TNN1 of at least 0.5:1 pre transplantation;
   xiv. ratios of expression levels of MYL2:MYL7. MYH7:MYH6, and TNNI3:TNN1 of at least 1:1 6 weeks post transplantation;
   xv. average cell area at least 1(K) microns squared; and/or
   xvi. sarcomere content of at least 30%,
518. The method according to any one of embodiments 1-517, wherein the cell or the population of cells are ABO blood group type O.
519. The method according to any one of embodiments 1-517, wherein the cell or the population of cells comprise a functional ABO A allele and/or a functional ABO B allele.
520. The method according to any one of embodiments 1-519, wherein the cell or the population of cells are Rhesus factor negative (Rh−).
521. The method according to any one of embodiments 1-519, wherein the cell or the population of cells are Rhesus factor positive (Rh+).
522. The method according to any preceding embodiment, wherein the cell or the population of cells are CD34+ cells.
523. The method according to any preceding embodiment, wherein the cell or the population of cells are differentiated cells derived from CD34+ cells.
524. The method according to any preceding embodiment, wherein the method further comprises determining reduction of the functions of the MHC Class I and/or MHC class II molecules of the cells.
525. The method according to embodiment 524, wherein the determining reduction of the functions comprises determining reduction of the expression of the MHC Class I and/or MHC class II molecules.
526. The method according to any preceding embodiment, wherein at least about 30% of cells in the population comprise modified cells.
527. The method according to embodiment 526, wherein:
   i. the population includes cells with hypoimmune gene modifications (HIP cells) that exhibit reduced expression of one or more molecules of the MHC class I and/or MHC class II molecules and optionally also exhibit increased expression of at least one tolerogenic factor; and
   ii. at least 30%, at least 40%, at least 50%, at least 60%, at least 70%, at least 80%, or at least 90% of cells in the population exhibit reduced expression of one or more molecules of the MHC class I and/or MHC class II molecules and optionally also exhibit increased expression of at least one tolerogenic factor.
528. The method of embodiment 526 or 527, wherein at least 5%, at least 10%, at least 20%, at least 30%, at least 40%, at least 50%, at least 60%, at least 70% of cells in the population of cells do not exhibit reduced expression of one or more molecules of the MHC class I and/or MHC class II molecules and optionally also do not exhibit increased expression of at least one tolerogenic factor.
529. The method according to any of embodiments 526-528, wherein 30-90%, 30-80%, 30-70%, 30-60%, 30-50% or 40-50% of cells in the population of cells are HIP cells that exhibit reduced expression of one or more molecules of the MHC class I and/or MHC class II molecules and optionally increased expression of at least one tolerogenic factor.
530. The method according to any of embodiments 526-529, wherein 70-100%, 80-100%, or 90-100% of cells in the population of cells express a CAR.
531. The method according to any preceding embodiment, wherein the cell or the population of cells, or progeny or differentiated cells derived from the cell or the population of cells have increased capability to evade NK cell mediated cytotoxicity upon administration to a subject as compared to a cell of the same type that does not comprise the one or more modifications.
532. The method according to any preceding embodiment, wherein the cell or the population of cells, or progeny or differentiated cells derived from the cell or the population of cells undergo reduced cell lysis by mature NK cells upon administration to a subject as compared to a cell of the same type that does not comprise the one or more modifications.
533. The method according to any preceding embodiment, wherein the cell or the population of cells, or progeny or differentiated cells derived from the cell or the population of cells induce a reduced immune response upon administration to a subject as compared to a cell of the same type that does not comprise the one or more modifications.
534. The method according to any preceding embodiment, wherein the cell or the population of cells, or progeny or differentiated cells derived from the cell or the population of cells induce a reduced systemic inflammatory response upon administration to a subject as compared to a cell of the same type that does not comprise the one or more modifications.
535. The method according to any preceding embodiment, wherein the cell or the population of cells, or progeny or differentiated cells derived from the cell or the population of cells induce a reduced local inflammatory response upon administration to a subject as compared to a cell of the same type that does not comprise the one or more modifications.
536. The method according to any preceding embodiment, wherein the cell or the population of cells, or progeny or differentiated cells derived from the cell or the population of cells induce reduced complement pathway activation upon administration to a subject as compared to a cell of the same type that does not comprise the one or more modifications.
537. The method according to any preceding embodiment, wherein the cell or the population of cells, or progeny or differentiated cells derived from the cell or the population of cells retain the ability to engraft and function upon administration to a subject.
538. The method according to any preceding embodiment, wherein the cell or the population of cells, or progeny or differentiated cells derived from the cell or the population of cells has increased ability to engraft and function upon administration to a subject as compared to a cell of the same type that does not comprise the one or more modifications.
539. A cell or a population of cells having the features of the cell or the population of cells evaluated, profiled, identified or selected according to any one of embodiments 1-538.
540. A cell having the features of a cell selected according to any one of embodiments 1-538.
541. A population of cells having the features of a population of cells selected according to any one of embodiments 1-538.
542. A cell or population of cells identified according to the method of any one of embodiments 1-538. 543. A cell or population of cells selected according to the method of any one of embodiments 1-538.
544. The population of cells according to any one of embodiments 539, or 541-543 wherein:
   i. the population includes cells with hypoimmune gene modifications (HIP cells) that exhibit reduced expression of one or more molecules of the MHC class I and/or MHC class II molecules and optionally also exhibit increased expression of at least one tolerogenic factor; and
   ii. at least 30%, at least 40%, at least 50%, at least 60%, at least 70%, at least 80%, or at least 90% of cells in the population exhibit reduced expression of one or more molecules of the MHC class I and/or MHC class II molecules and optionally also exhibit increased expression of at least one tolerogenic factor.
545. The population of cells according to embodiment 544, wherein at least 5%, at least 10%, at least 20%, at least 30%, at least 40%, at least 50%, at least 60%, at least 70% of cells in the population of cells do not exhibit reduced expression of one or more molecules of the MHC class I and/or MHC class II molecules and optionally also do not exhibit increased expression of at least one tolerogenic factor.
546. The population of cells according to embodiment 544 or 545, wherein 30-90%, 30-80%, 30-70%, 30-60%, 30-50% or 40-50% of cells in the population of cells are HIP cells that exhibit reduced expression of one or more molecules of the MHC class I and/or MHC class II molecules and optionally increased expression of at least one tolerogenic factor.
547. The population of cells according to any one of embodiments 544-546, wherein 70-100%, 80-100%, or 90-100% of cells in the population of cells express a CAR.
548. A method of generating a cell or a population of cells as defined in embodiments 1-538 comprising
   a. obtaining a cell according to any one of embodiments 1-538; and
   b. introducing one or more modifications as defined in any one of embodiments 1-538 into the cell.
549. The method according to embodiment 548, wherein the method further comprises selecting the cell from a population of cells based on the presence or expression level of one or more of the modifications.
550. The method according to embodiment 548 or 549, wherein the cell is a stem cell or a progenitor cell and the method further comprises differentiating the stem cell or the progenitor cell.
551. The method according to embodiment 548 or 549, wherein the cell is a pluripotent stem cell or a progeny thereof and the method comprises differentiating the pluripotent stem cell or progeny thereof.
552. The method according to embodiment 548 or 549, wherein the cell is a primary cell.
553. The method according to any one of embodiments 548-552, wherein the method comprises introducing one or more gene edits into the genome of the cell.
554. The method according to embodiment 553, wherein the one or more gene edits are introduced into the genome of the cell by non-targeted insertion.
555. The method according to embodiment 553, wherein the one or more gene edits are introduced into the genome of the cell by targeted insertion.
556. The method according to any one of embodiments 553-555, wherein the one or more gene edits are introduced into one or more genes encoding the one or more molecules of any of embodiments 1-538.
557. The method according to embodiment 556, wherein the engineered cell has increased expression of the one or more molecules encoded by the one or more edited genes.
558. The method according to embodiment 556 or embodiment 557, wherein the engineered cell has reduced expression of the one or more molecules encoded by the one or more edited genes.
559. The method according to any one of embodiments 553-558, wherein the one or more gene edits are introduced into the genome of cell using at least one of the genome editing complexes of any of embodiments 405-421.
560. The method according to any one of embodiments 553-559, wherein the one or more gene edits are introduced into the genome of cell at one or more target genomic loci selected from the group consisting of an albumin gene locus, an ABO gene locus, a B2M gene locus, a CIITA gene locus, a CCR5 gene locus, a CD142 gene locus, a CLYBL gene locus, a CXCR4 gene locus, an F3 gene locus, a FUT1 gene locus, an HMGB1 gene locus, a KDM5D gene locus, an LRP1 gene locus, a MIC-A gene locus, a MIC-B gene locus, a PPP1R12C (also known as AAVS1) gene locus, an RHD gene locus, a ROSA26 gene locus, a safe harbor gene locus, a SHS231 locus, a TAP1 gene locus, a TRAC gene locus, and a TRBC gene locus.
561. A modified cell or population of cells produced by the method according to any one of embodiments 1-538.
562. A method of producing a composition comprising a cell or a population of cells comprising
   a. obtaining a cell or a population of cells according to any one of embodiments 539-547 and 561; and
   b. formulating the composition comprising the cell or the population of cells.
563. A method of producing a composition comprising a cell or a population of cells comprising
   a. obtaining a cell or the population of cells according to any one of embodiments 539-547 and 561;
   b. introducing the one or more modifications of any of embodiments 1-538 into the cell or the population of cells;
   c. optionally selecting the modified cell or selecting the population of modified cells
      i. based on a level of the one or more modifications; and/or
      ii. the evaluation/profiling/identifying/selection methods according to embodiments 1-538; and
   d. formulating the composition comprising the modified cell or the population of modified cells.
564. A method of producing a composition comprising the cell or the population according to any one of embodiments 539-547 and 561 comprising
   a. obtaining a cell or a population of cells according to any one of embodiments 1-538;
   b. introducing the one or more modifications of any of embodiments 1-538 into the cell or the population of cells;
   c. selecting the modified cell or selecting the population of modified cells:
      i. based on a level of the one or more modifications; and/or
      ii. the evaluation/profiling/identifying/selection methods according to embodiments 1-538; and
   d. formulating the composition comprising the selected modified cell or the selected population of modified cells.
565. The method according to embodiment 562-564, wherein selecting based on a level of the one or more modifications comprises selecting based on one or more modified molecules having reduced expression in the modified cell or the population of modified cells.
566. The method according to any one of embodiments 562-565, wherein selecting based on a level of the one or more modifications comprises selecting based on one or more modified molecules having increased expression in the modified cell or the population of modified cells.
567. The method according to any one of embodiments 562-566, wherein selecting based on a level of the one or more modifications comprises selecting based on cell surface expression of the one or more modified molecules in any of embodiments 176-388.
568. The method according to any of embodiments 562-567, wherein the method comprises formulating the composition in a pharmaceutically acceptable additive, carrier, diluent, or excipient.
569. The method according to embodiment 568, wherein the pharmaceutically acceptable additive, carrier, diluent, or excipient comprises a pharmaceutically acceptable buffer.
570. The method according to embodiment 569, wherein the pharmaceutically acceptable buffer comprises neutral buffer saline or phosphate buffered saline.
571. The method according to any of embodiments 562-570, wherein the method comprises formulating the composition with Plasma-Lyte A®, dextrose, dextran, sodium chloride, human serum albumin (HSA), dimethylsulfoxide (DMSO), or a combination thereof.
572. The method according to any of embodiments 562-571, wherein the method comprises formulating the composition with a cryoprotectant.
573. The method according to any of embodiments 562-572, wherein the method comprises formulating the composition in a serum-free cryopreservation medium comprising a cryoprotectant.
574. The method according to embodiment 572 or embodiment 573, wherein the cryoprotectant comprises DMSO.
575. The method according to embodiment 574, wherein the serum-free cryopreservation medium comprises about 5% to about 10% DMSO (v/v).
576. The method according to embodiment 574, wherein the serum-free cryopreservation medium comprises about 10% DMSO (v/v).
577. The method according to any of embodiments 562-576, wherein the method further comprises storing the composition in a container.
578. The method according to any of embodiments 562-577, wherein the method further comprises thawing the cell before step (b).
579. The method according to any of embodiments 562-578, wherein the method further comprises freezing the modified cell, the population of modified cells, or the composition.
580. The method according to embodiment 579, wherein the modified cell or the population of modified cells are frozen after step (b).
581. The method according to embodiment 580, wherein the modified cell or the population of modified cells are thawed before step (c).
582. The method according to embodiment 579, wherein the modified cell or the population of modified cells are frozen after step (c).
583. The method according to embodiment 580, wherein the modified cell or the population of modified cells are thawed before step (d).
584. The method according to embodiment 579, wherein the modified cell or the population of modified cells are frozen after step (c).
585. The method according to any of embodiments 562-584, wherein the composition is frozen after step (d).
586. A composition comprising the cell or the population of cells according to any of embodiments 539-547 and 561.
587. A composition according to embodiment 586, wherein:
   i. the population includes cells with hypoimmune gene modifications (HIP cells) that exhibit reduced expression of one or more molecules of the MHC class I and/or MHC class II molecules and optionally also exhibit increased expression of at least one tolerogenic factor; and
   ii. at least 30%, at least 40%, at least 50%, at least 60%, at least 70%, at least 80%, or at least 90% of cells in the population exhibit reduced expression of one or more molecules of the MHC class I and/or MHC class II molecules and optionally also exhibit increased expression of at least one tolerogenic factor.
588. A composition according to embodiment 586 or 587, wherein at least 5%, at least 10%, at least 20%, at least 30%, at least 40%, at least 50%, at least 60%, at least 70% of cells in the population of cells do not exhibit reduced expression of one or more molecules of the MHC class I and/or MHC class II molecules and optionally also do not exhibit increased expression of at least one tolerogenic factor.
589. A composition according to any one of embodiments 586-588, wherein 30-90%, 30-80%, 30-70%, 30-60%, 30-50% or 40-50% of cells in the population of cells are HIP cells that exhibit reduced expression of one or more molecules of the MHC class I and/or MHC class II molecules and optionally increased expression of at least one tolerogenic factor.
590. A composition according to any one of embodiments 586-589, wherein 70-100%, 80-100%, or 90-100% of cells in the population of cells express a CAR.
591. A composition produced by the method according to any one of embodiments 562-585.
592. The composition according to any one of embodiments 586-591, wherein the composition comprises a pharmaceutically acceptable additive, carrier, diluent, or excipient.
593. The composition of any of embodiments 586-592, wherein the composition is sterile.
594. A container comprising the composition according to any of embodiments 586-593.
595. The container according to embodiment 594, wherein the container is a sterile bag.
596. The container according to embodiment 595, wherein the sterile bag is a cryopreservation-compatible bag.
597. A kit comprising the composition of any of embodiments 586-593 or the container of any of embodiments 594-596.
598. The kit according to embodiment 597, wherein the kit further comprises instructions for using the cells or the population of cells.
599. Method of making a cell therapy product comprising providing a cell or the population of cells that have been evaluated, profiled, identified or selected according to a method defined in embodiments 1-538 and manufacturing a cell therapy product therefrom.
600. A method of making a cell therapy product according to embodiment 599, wherein:
   i. the population includes cells with hypoimmune gene modifications (HIP cells) that exhibit reduced expression of one or more molecules of the MHC class I and/or MHC class II molecules and optionally also exhibit increased expression of at least one tolerogenic factor; and
   ii. at least 30%, at least 40%, at least 50%, at least 60%, at least 70%, at least 80%, or at least 90% of cells in the population exhibit reduced expression of one or more molecules of the MHC class I and/or MHC class II molecules and optionally also exhibit increased expression of at least one tolerogenic factor.
601. The method of embodiment 599 or 600, wherein at least 5%, at least 10%, at least 20%, at least 30%, at least 40%, at least 50%, at least 60%, at least 70% of cells in the population of cells do not exhibit reduced expression of one or more molecules of the MHC class I and/or MHC class II molecules and optionally also do not exhibit increased expression of at least one tolerogenic factor.
602. The method according to any one of embodiments 599-601, wherein 30-90%, 30-80%, 30-70%, 30-60%, 30-50% or 40-50% of cells in the population of cells are HIP cells that exhibit reduced expression of one or more molecules of the MHC class I and/or MHC class II molecules and optionally increased expression of at least one tolerogenic factor.
603. The method according to any one of embodiments 599-602, wherein 70-100%, 80-100%, or 90-100% of cells in the population of cells express a CAR.
   x.
604. The method according to any one of embodiments 599-603, wherein the method defined in embodiments 1-538 is performed by a third party.
605. The method according to any one of embodiments 599-603, wherein the manufacturing is performed by a third party.
606. Method of evaluating, profiling, identifying or selecting a cell or a population of cells according to embodiments 1-538 and manufacturing a cell therapy product therefrom.
607. Use of a cell or a population of cells that have been evaluated, profiled, identified or selected according to a method defined in embodiments 1-538 for manufacturing a cell therapy product.
608. Method of enhancing cell function of a cell therapy comprising evaluating, profiling, identifying or selecting a cell or a population of cells according to a method defined in embodiments 1-538 and administering the cell therapy to a subject.
609. Method of enhancing cell function of a cell therapy comprising evaluating, profiling, identifying or selecting a cell or a population of cells according to a method defined in embodiments 1-538 and making a cell therapy product therefrom and administering the cell therapy product to a subject.
610. The method according to embodiment 608 or embodiment 609, wherein enhancing cell function comprises enhancing durability of cell response.
611. The method according to any one of embodiments 608-610, wherein enhancing cell function comprises promoting cell persistence.
612. The method according to any one of embodiments 608-611, wherein enhancing cell function comprises promoting engraftment.
613. The method according to any one of embodiments 608-612, wherein enhancing cell function comprises enhancing potency.
614. The method according to any one of embodiments 608-613, wherein enhancing cell function comprises enhancing hypoimmunogenicity.
615. Method of improving likelihood of therapeutic effect of a cell therapy comprising evaluating, profiling, identifying or selecting a cell or a population of cells according to a method defined in embodiments 1-538 and administering the cell therapy to a subject.
616. Method of improving likelihood of therapeutic effect of a cell therapy comprising evaluating, profiling, identifying or selecting a cell or a population of cells according to a method defined in embodiments 1-538 and making a cell therapy product therefrom and administering the cell therapy product to a subject.
617. The method according to embodiment 615 or embodiment 616, wherein improving likelihood of therapeutic effect comprises enhancing durability of response.
618. The method according to any one of embodiments 615-617, wherein improving likelihood of therapeutic effect comprises promoting cell persistence.
619. The method according to any one of embodiments 615-618, wherein improving likelihood of therapeutic effect comprises promoting engraftment.
620. The method according to any one of embodiments 615-618, wherein improving likelihood of therapeutic effect comprises enhancing potency.
621. The method according to any one of embodiments 615-618, wherein improving likelihood of therapeutic effect comprises enhancing hypoimmunogenicity.
622. A method to determine whether to administer autologous cell therapy to a subject in need of cell therapy, the method comprising a method of profiling an autologous sample of cells from the subject according to any one of embodiments 1-538, wherein the determination is made to administer autologous cell therapy to the subject if
   i. the autologous sample of cells from the subject is categorised as above a reference value or at a reference value, particularly when categorised as above a reference value;
   ii. the gene expression profile of the autologous sample of cells is comparable to a reference signature gene expression profile; and/or
   iii. the AUC is less than 20 or between 20 and 100 at high dose, or the AUC is less than 11000 at low dose.
623. A method to determine whether to administer allogeneic cell therapy to a subject in need of cell therapy, the method comprising a method of profiling an autologous sample of cells from the subject according to any one of embodiments 1-538, wherein the determination is made to administer allogeneic cell therapy to the subject if
   iv. the autologous sample of cells from the subject is categorised as below a reference value or at a reference value, particularly when categorised as below a reference value;
   v. the gene expression profile of the autologous sample of cells is not comparable to a reference signature gene expression profile; and/or
   vi. the AUC is more than 100 at high dose, or the AUC is more than 11000 at low dose.
624. A method to determine whether to administer in vivo CAR T therapy using a T cell targeted viral vector to a subject in need thereof, the method comprising a method of profiling an autologous sample of cells from the subject according to any one of embodiments 1-538, wherein the determination is made to administer in vivo CAR T therapy using a T cell targeted viral vector to the subject if
   iv. the autologous sample of cells from the subject is categorised as below a reference value or at a reference value, particularly when categorised as below a reference value;
   v. the gene expression profile of the autologous sample of cells is not comparable to a reference signature gene expression profile; and/or
   vi. the AUC is more than 100 at high dose, or the AUC is more than 11000 at low dose.
625. The method according to any one of embodiments 622-624, wherein the reference value is selected from the group consisting of: a reference value for predicted cell function, a reference value for defining the usability as a donor, a reference value for growth, a reference value for growth rate, a reference value for durability of cell growth, a reference value for durability of cell response, a reference value for cytokine production, a reference value for bulk cytokine production, a reference value for PSi, and a reference value for MSi.
626. The method according to any one of embodiments 622-625, wherein the reference value is selected from the group consisting of: an average value, a median value, a mean value, a value range, a pre-set value, a pre-determined value, an experimentally determined value, a computed value, and a cell type specific value.
627. The method according to any one of embodiments 622-626, wherein the reference value is determined in a reference cell or population of cells of the same cell type or subtypes as the cell or the population of cells.
628. The method according to any one of embodiments 622-626, wherein the reference value is determined in a reference cell or population of cells of a different cell type or subtypes as the cell or the population of cells.
629. The method according to any one of embodiments 622-628, wherein the reference value is determined in a reference cell or population of cells that do not comprise the one or more modifications.
630. The method according to any one of embodiments 622-628, wherein the reference value is determined in a reference cell or population of cells that comprise the one or more modifications.
631. Use of an assay for evaluating cells for predicted function to predict the in vivo function of a cell or a population of cells for administration to a subject as a cell therapy.
632. Use of an assay for evaluating cells for predicted function to predict the in vivo function of a cell or a population of cells for making a cell therapy product.
633. The use according to embodiment 631 or embodiment 632, wherein the assay comprises at least one of an in vitro assay, an in vivo assay, an immune assay, a cell activity assay, a cell avidity assay, a cell proliferation assay, a cell cytotoxicity assay, a cellular stress assay, a tumor challenge assay, an expression assay, a cytokine production assay, a transcriptomic profiling assay, a proteomic profiling assay, a genomic profiling assay, a genomic stability assay, an epigenetic profiling assay, a cell developmental potential profiling assay, a cell subtyping assay; and/or a cell receptor profiling assay.
634. The use according to any one of embodiments 631-633, wherein the predicted cell function is at least one of the cell functions selected from the group consisting of: cell persistence, engraftment, durability of cell response, potency and hypoimmunogenicity.
635. The use according to any one of embodiments 631-634, wherein the predicted cell function is evaluated by assaying at least one of the cell parameters comprising:
   s) cell activation;
   t) cell polyfunctionality or cell multifunctionality;
   u) cell cytotoxicity;
   v) cell growth rate;
   w) durability of cell growth;
   x) durability of cell response;
   y) the cell's ability to elicit adaptive and innate immune responses
   z) characteristics associated with particular cell type (e.g., cell marker characterization, cell cytokine production, antibody production);
   aa) cell cytokine production;
   bb)cell viability;
   cc) cell safety attributes; and/or
   dd)cell impurity level(s).
636. The use according to any one of embodiments 631-635, wherein the assay is as defined in any of embodiments 1-538, the predicted cell function is as defined in any of embodiments 1-538 and/or the cells are as defined in any of embodiments -1-547 or 561. 637. Use of profiling the donor capability of a cell or the population of cells for categorising the cell or the population of cells for cell therapy.
638. The use according to embodiment 637, wherein the profiling comprises evaluating cells for predicted function, wherein the evaluating is as defined in any of embodiments 1-538.
639. The use according to embodiment 637 or embodiment 638, wherein the profiling comprises a scale as defined in any of embodiments 1-538.
640. The use according to embodiment 638 or embodiment 639, wherein the predicted cell function is as defined in any of embodiments 1-538 and/or the cells are as defined in any of embodiments 1-547 or 561.
641. Use of real-time quantitative live-cell analysis for measuring (i) growth rate and/or (ii) durability of cell growth and/or (iii) durability of cell response of a cell or the population of cells to predict the in vivo functionality of the cell or the population of cells for administration to a subject as a cell therapy.
642. Use of a multiplex cytokine detection technique for measuring cytokine production for a panel of cytokines in a cell or the population of cells to predict the in vivo functionality of the cell or the population of cells as a cell therapy, optionally wherein the panel of cytokines comprises at least one (optionally all) of GM-CSF, GzmA, GzmB, IFNg, TNFa, Il2, Il6, Il17A, Il1b, and Il11RA.
643. Use of single cell cytokine profiling on a cell or the population of cells to predict the in vivo functionality of the cell or the population of cells as a cell therapy.
644. Use of gene expression profiling on a cell or the population of cells to determine resting state activation level to predict the in vivo functionality of the cell or the population of cells as a cell therapy.
645. Use of gene expression profiling on a cell or the population of cells to determine activated state activation level to predict the in vivo functionality of the cell or the population of cells as a cell therapy.
646. A cell or a population of cells as defined in any one of embodiments 539-547 and 561for use in therapy
647. A composition comprising a cell or a population of cells as defined in any one of embodiments 539-547 and 561, for use in therapy
648. A cell or a population of cells as defined in in any one of embodiments 539-547 and
561, for use in a method of treating a disease or condition.
649. A composition as defined in any one of embodiments 586-593, for use in a method of treating a disease or condition.
650. The population of cells or composition comprising a population of cells for use according to embodiment 646-649 wherein:
   iii. the population includes cells with hypoimmune gene modifications (HIP cells) that exhibit reduced expression of one or more molecules of the MHC class I and/or MHC class II molecules and optionally also exhibit increased expression of at least one tolerogenic factor; and
   iv. at least 30%, at least 40%, at least 50%, at least 60%, at least 70%, at least 80%, or at least 90% of cells in the population exhibit reduced expression of one or more molecules of the MHC class I and/or MHC class II molecules and optionally also exhibit increased expression of at least one tolerogenic factor.
651. The population of cells or composition comprising a population of cells for use according to embodiment 646-650, wherein at least 5%, at least 10%, at least 20%, at least
30%, at least 40%, at least 50%, at least 60%, at least 70% of cells in the population of cells do not exhibit reduced expression of one or more molecules of the MHC class I and/or MHC class II molecules and optionally also do not exhibit increased expression of at least one tolerogenic factor.
652. The population of cells or composition comprising a population of cells for use according to embodiment 646-651, wherein 30-90%, 30-80%, 30-70%, 30-60%, 30-50% or 40-50% of cells in the population of cells are HIP cells that exhibit reduced expression of one or more molecules of the MHC class I and/or MHC class II molecules and optionally increased expression of at least one tolerogenic factor.
653. The population of cells or composition comprising a population of cells for use according to embodiment 646-652, wherein 70-100%, 80-100%, or 90-100% of cells in the population of cells express a CAR.
654. A method of treating a disease or condition in a subject comprising administering to the subject a cell or a population of cells selected according to the method according to any one of embodiments 1-538.
655. A method of treating a disease or condition in a subject comprising administering to the subject a cell or a population of cells as defined in any one of embodiments 539-547 and 561.
656. A method of treating a disease or condition in a subject comprising administering to the subject a composition as defined in any one of embodiments 586-593.
657. The method according to any one of embodiments 654-656, wherein the subject is in need of therapy.
658. The cell or the population of cells for use according to any one of embodiments 648 and 650-653, the composition for use according to any one of embodiments 649-653, or the method according to any one of embodiments 654-657, wherein the disease or condition is a cellular deficiency.
659. The cell or the population of cells for use according to embodiment 658, the composition for use according to embodiment 658, or the method according to embodiment 658, wherein the condition or disease is selected from the group consisting of diabetes, cancer, vascularization disorders, ocular disease, thyroid disease, skin diseases, and liver diseases.
660. The cell or the population of cells for use according to embodiment 658, the composition for use according to embodiment 658, or the method according to embodiment 658, wherein the condition or disease is associated with diabetes or is diabetes, optionally wherein the diabetes is Type I diabetes.
661. The cell or the population of cells for use according to embodiment 660, the composition for use according to embodiment 660, or the method according to embodiment 660, wherein the population of cells is a population of islet cells, including beta islet cells.
662. The cell or the population of cells for use according to embodiment 661, the composition for use according to embodiment 661, or the method according to embodiment 661, wherein the islet cells are selected from the group consisting of an islet progenitor cell, an immature islet cell, and a mature islet cell.
663. The cell or the population of cells for use according to embodiment 658, the composition for use according to embodiment 658, or the method according to embodiment 658, wherein the condition or disease is associated with a vascular condition or disease or is a vascular condition or disease.
664. The cell or the population of cells for use according to embodiment 663, the composition for use according to embodiment 663, or the method according to embodiment 663, wherein the cell or the population of cells comprises an endothelial cell.
665. The cell or the population of cells for use according to embodiment 658, the composition for use according to embodiment 658, or the method according to embodiment 658, wherein the condition or disease is associated with autoimmune thyroiditis or is autoimmune thyroiditis.
666. The cell or the population of cells for use according to embodiment 665, the composition for use according to embodiment 665, or the method according to embodiment 665, wherein the cell or the population of cells comprise a thyroid progenitor cell.
667. The cell or the population of cells for use according to embodiment 658, the composition for use according to embodiment 658, or the method according to embodiment 658, wherein the condition or disease is associated with a liver disease or is liver disease.
668. The cell or the population of cells for use according to embodiment 667 the composition for use according to embodiment 667, or the method according to embodiment 667, wherein the liver disease comprises cirrhosis of the liver.
669. The cell or the population of cells for use according to embodiment 667 or embodiment 668, the composition for use according to embodiment 667 or embodiment
668, or the method according to embodiment 667 or embodiment 668, wherein the cell or the population of cells comprise a hepatocyte or a hepatic progenitor cell.
670. The cell or the population of cells for use according to embodiment 658, the composition for use according to embodiment 658, or the method according to embodiment 658, wherein the condition or disease is associated with a corneal disease or is corneal disease.
671. The cell or the population of cells for use according to embodiment 670, the composition for use according to embodiment 670, or the method according to embodiment 670, wherein the corneal disease is Fuchs dystrophy or congenital hereditary endothelial dystrophy.
672. The cell or the population of cells for use according to embodiment 670 or embodiment 671, the composition for use according to embodiment 670 or embodiment
671, or the method according to embodiment 670 or embodiment 671, wherein cell or the population of cells comprise a corneal endothelial progenitor cell or a corneal endothelial cells.
673. The cell or the population of cells for use according to embodiment 658, the composition for use according to embodiment 658, or the method according to embodiment 658, wherein the condition or disease is associated with a kidney disease or is kidney disease.
674. The cell or the population of cells for use according to embodiment 673, the composition for use according to embodiment 673, or the method according to embodiment 673, wherein the cell or the population of cells comprise a renal precursor cell or a renal cell.
675. The cell or the population of cells for use according to any one of embodiments 648 and 650-653, the composition for use according to any one of embodiments 649-653, or the method according to any one of embodiments 654-657, wherein the disease or condition is a disease associated with cancer or cancer.
676. The cell or the population of cells for use according to embodiment 675, the composition for use according to embodiment 675, or the method according to embodiment 675, wherein the cancer is selected from the group consisting of B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.
677. The cell or the population of cells for use according to embodiment 675 or embodiment 676, the composition for use according to embodiment 675 or embodiment
676, or the method according to embodiment 675 or embodiment 676, wherein the cell or the population of cells comprise a T cell, an NK cell, or an NKT cell.
678. The cell or the population of cells for use according to any one of embodiments 675-677, the composition for use according to any one of embodiments 675-677, or the method according to any one of embodiments 675-677, wherein the CAR is a CD19 CAR and the disease is CD19+ B-cell leukaemia.
679. The cell or the population of cells for use according to embodiment 658, the composition for use according to embodiment 658, or the method according to embodiment 658, wherein the condition or disease is associated with a hematopoietic disease or disorder or is a hematopoietic disease or disorder.
680. The cell or the population of cells for use according to embodiment 679, the composition for use according to embodiment 679, or the method according to embodiment 679, wherein the hematopoietic disease or disorder is myelodysplasia, aplastic anemia, Fanconi anemia, paroxysmal nocturnal hemoglobinuria, Sickle cell disease, Diamond Blackfan anemia, Schachman Diamond disorder, Kostmann's syndrome, chronic granulomatous disease, adrenoleukodystrophy, leukocyte adhesion deficiency, hemophilia, thalassemia, beta-thalassemia, leukaemia such as acute lymphocytic leukemia (ALL), acute myelogenous (myeloid) leukemia (AML), adult lymphoblastic leukaemia, chronic lymphocytic leukemia (CLL), B-cell chronic lymphocytic leukemia (B-CLL), chronic myeloid leukemia (CML), juvenile chronic myelogenous leukemia (CML), and juvenile myelomonocytic leukemia (JMML), severe combined immunodeficiency disease (SCID), X-linked severe combined immunodeficiency, Wiskott-Aldrich syndrome (WAS), adenosine-deaminase (ADA) deficiency, chronic granulomatous disease, Chediak-Higashi syndrome, Hodgkin's lymphoma, non-Hodgkin's lymphoma (NHL) or AIDS.
681. The cell or the population of cells for use according to embodiment 658, the composition for use according to embodiment 658, or the method according to embodiment 658, wherein the condition or disease is associated with leukemia or myeloma or is leukemia or myeloma.
682. The cell or the population of cells for use according to embodiment 658, the composition for use according to embodiment 658, or the method according to embodiment 658, wherein the condition or disease is associated with an autoimmune disease or condition or is an autoimmune disease or condition.
683. The cell or the population of cells for use according to embodiment 682, the composition for use according to embodiment 682, or the method according to embodiment 682, wherein the autoimmune disease or condition is acute disseminated encephalomyelitis, acute hemorrhagic leukoencephalitis, Addison's disease, Agammaglobulinemia, Alopecia areata, amyotrophic lateral sclerosis, ankylosing spondylitis, antiphospholipid syndrome, antisynthetase syndrome, atopic allergy, autoimmune aplastic anemia, autoimmune cardiomyopathy, autoimmune enteropathy, autoimmune hemolytic anemia, autoimmune hepatitis, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticaria, autoimmune uveitis, Balo disease, Balo concentric sclerosis, Bechets syndrome, Berger's disease, Bickerstaff's encephalitis, Blau syndrome, bullous pemphigoid, cancer, Castleman's disease, celiac disease, chronic inflammatory demyelinating polyneuropathy, chronic recurrent multifocal osteomyelitis, Churg-Strauss syndrome, cicatricial pemphigoid, Cogan syndrome, cold agglutinin disease, complement component 2 deficiency, cranial arteritis, CREST syndrome, Crohn's disease. Cushing's syndrome, cutaneous leukocytoclastic angiitis, Dego's disease, Dercum's disease, dermatitis herpetiformis, dermatomyositis, diabetes mellitus type 1, diffuse cutaneous systemic sclerosis, Dressler's syndrome, discoid lupus erythematosus, eczema, enthesitis-related arthritis, eosinophilic fasciitis, eosinophilic gastroenteritis, epidermolysis bullosa acquisita, erythema nodosum, essential mixed cryoglobulinemia, Evan's syndrome, firodysplasia ossificans progressiva, fibrosing aveolitis, gastritis, gastrointestinal pemphigoid, giant cell arteritis, glomerulonephritis, goodpasture's syndrome, Grave's disease, Guillain-Barre syndrome (GBS), Hashimoto's encephalitis, Hashimoto's thyroiditis, hemolytic anaemia, Henoch-Schonlein purpura, herpes gestationis, hypogammaglobulinemia, idiopathic inflammatory demyelinating disease, idiopathic pulmonary fibrosis, idiopathic thrombocytopenic purpura, IgA nephropathy, inclusion body myositis, inflammatory demyelinating polyneuropathy, interstitial cystitis, juvenile idiopathic arthritis, juvenile rheumatoid arthritis, Kawasaki's disease, Lambert-Eaton myasthenic syndrome, leukocytoclastic vasculitis, lichen planus, lichen sclerosus, linear IgA disease (LAD), Lou Gehrig's disease, lupoid hepatitis, lupus erythematosus, Majeed syndrome, Meniere's disease, microscopic polyangiitis, Miller-Fisher syndrome, mixed connective tissue disease, morphea, Mucha-Habermann disease, multiple sclerosis, myasthenia gravis, myositis, neuropyelitis optica, neuromyotonia, ocular cicatricial pemphigoid, opsoclonus myoclonus syndrome, ord thyroiditis, palindromic rheumatism, paraneoplastic cerebellar degeneration, paroxysmal nocturnal hemoglobinuria (PNH), Parry Romberg syndrome, Parsonnage-Turner syndrome, pars planitis, pemphigus, pemphigus vulgaris, permicious anemia, perivenous encephalomyelitis, POEMS syndrome, polyarteritis nodosa, polymyalgia rheumatica, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis. Reiter's syndrome, restless leg syndrome, retroperitoneal fibrosis, rheumatoid arthritis, rheumatoid fever, sarcoidosis, Schmidt syndrome, Schnitzler syndrome, scleritis, scleroderma, Sjogren's syndrome, spondylarthropathy, Still's disease, stiff person syndrome, subacute bacterial endocarditis, Susac's syndrome, Sweet's syndrome, Sydenham chorea, sympathetic ophthalmia, Takayasu's arteritis, temporal arteritis, Tolosa-Hunt syndrome, transverse myelitis, ulcerative colitis, undifferentiated connective tissue disease, undifferentiated spondylarthropathy, vasculitis, vitiligo or Wegener's granulomatosis.
684. The cell or the population of cells for use according to any one of embodiments 679-683, the composition for use according to any one of embodiments 679-683, or the method according to any one of embodiments 679-683, wherein the cell or the population of cells comprises a hematopoietic stem cell (HSC) or a derivative thereof.
685. The cell or the population of cells for use according to embodiment 658, the composition for use according to embodiment 658, or the method according to embodiment 658, wherein the condition or disease is associated with Parkinson's disease, Huntington disease, multiple sclerosis, a neurodegenerative disease or condition, attention deficit hyperactivity disorder (ADHD), Tourette Syndrome (TS), schizophrenia, psychosis, depression, a neuropsychiatric disorder stroke, or amyotrophic lateral sclerosis (ALS), or wherein the disease or condition is Parkinson's disease, Huntington disease, multiple sclerosis, a neurodegenerative disease or condition, attention deficit hyperactivity disorder (ADHD), Tourette Syndrome (TS), schizophrenia, psychosis, depression, a neuropsychiatric disorder stroke, or amyotrophic lateral sclerosis (ALS).
686. The cell or the population of cells for use according to embodiment 685, the composition for use according to embodiment 685, or the method according to embodiment 685, wherein the cell or the population of cells comprise a neural cell or a glial cell.
687. The cell or the population of cells for use according to any one of embodiments 648, 650-653 and 657-686, the composition for use according to any one of embodiments 649-653 and 657-686, or the method according to any one of embodiments 654-686, wherein the cell or the population of cells are expanded and cryopreserved prior to administration.
688. The cell or the population of cells for use according to any one of embodiments 648, 650-653 and 657-687, the composition for use according to any one of embodiments 649-653 and 657-687, or the method according to any one of embodiments 654-687, wherein the method comprises intravenous injection, intramuscular injection, intravascular injection, or transplantation of the cell, the population of cells, or the composition.
689. The cell or the population of cells for use according to embodiment 688, the composition for use according to embodiment 688, or the method according to embodiment 688, wherein transplantation comprises intravascular injection or intramuscular injection.
690. The cell or the population of cells for use according to any one of embodiments 648, 650-653 and 657-689, the composition for use according to any one of embodiments 649-653 and 657-689, or the method according to any one of embodiments 654-689, wherein the method further comprises administering one or more immunosuppressive agents to the subject.
691. The cell or the population of cells for use according to any one of embodiments 648, 650-653 and 657-690, the composition for use according to any one of embodiments 649-653 and 657-690, or the method according to any one of embodiments 654-690, wherein the subject has been administered one or more immunosuppressive agents.
692. The cell or the population of cells for use according to any one of embodiments 648, 650-653 and 657-689, the composition for use according to any one of embodiments 649-653 and 657-689, or the method according to any one of embodiments 654-689, wherein the method does not comprise administering one or more immunosuppressive agents to the subject.
693. The cell or the population of cells for use according to any one of embodiments 648, 650-653. 657-689 and 692, the composition for use according to any one of embodiments 649-653, 657-689 and 692, or the method according to any one of embodiments 654-689 and 692, wherein the subject is not immunosuppressed i.e., has not been administered one or more immunosuppressive agents.
694. The cell or the population of cells for use according to any one of embodiments 690-693, the composition for use according to any one of embodiments 690-693, or the method according to any one of embodiments 690-693, wherein the one or more immunosuppressive agents are a small molecule or an antibody.
695. The cell or the population of cells for use according to any one of embodiments 690-694, the composition for use according to any one of embodiments 690-694, or the method according to any one of embodiments 690-694, wherein the one or more immunosuppressive agents are selected from the group consisting of cyclosporine, azathioprine, mycophenolic acid, mycophenolate mofetil, a corticosteroids, prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin (thymosin-α), an immunomodulatory agent, and an immunosuppressive antibody.
696. The cell or the population of cells for use according to any one of embodiments 690-695690-695, the composition for use according to any one of embodiments 690-695690-695, or the method according to any one of embodiments 690-695690-695, wherein the one or more immunosuppressive agents comprise cyclosporine.
697. The cell or the population of cells for use according to any one of embodiments 690-695690-695, the composition for use according to any one of embodiments 690-695, or the method according to any one of embodiments 690-695, wherein the one or more immunosuppressive agents comprise mycophenolate mofetil.
698. The cell or the population of cells for use according to any one of embodiments 690-695, the composition for use according to any one of embodiments 690-695, or the method according to any one of embodiments 690-695, wherein the one or more immunosuppressive agents comprise a corticosteroid.
699. The cell or the population of cells for use according to any one of embodiments 690-695, the composition for use according to any one of embodiments 690-695, or the method according to any one of embodiments 690-695, wherein the one or more immunosuppressive agents comprise cyclophosphamide.
700. The cell or the population of cells for use according to any one of embodiments 690-695, the composition for use according to any one of embodiments 690-695, or the method according to any one of embodiments 690-695, wherein the one or more immunosuppressive agents comprise rapamycin.
701. The cell or the population of cells for use according to any one of embodiments 690-695, the composition for use according to any one of embodiments 690-695, or the method according to any one of embodiments 690-695, wherein the one or more immunosuppressive agents comprise tacrolimus (FK-506).
702. The cell or the population of cells for use according to any one of embodiments 690-695, the composition for use according to any one of embodiments 690-695, or the method according to any one of embodiments 690-695, wherein the one or more immunosuppressive agents comprise anti-thymocyte globulin.
703. The cell or the population of cells for use according to any one of embodiments 690-695, the composition for use according to any one of embodiments 690-695, or the method according to any one of embodiments 690-695, wherein the one or more immunosuppressive agents are one or more immunomodulatory agents.
704. The cell or the population of cells for use according to embodiment 703, the composition for use according to embodiment 703, or the method according to embodiment 703, wherein the one or more immunomodulatory agents are a small molecule or an antibody.
705. The cell or the population of cells for use according to embodiment 703 or embodiment 704, the composition for use according to embodiment 703 or embodiment 704, or the method according to embodiment 703 or embodiment 704, wherein the antibody binds to one or more receptors or ligands selected from the group consisting of p75 of the IL-2 receptor, MHC, CD2, CD3, CD4, CD7, CD28, B7, CD40, CD45, IFN-gamma, TNF-alpha, IL-4, IL-5, IL-6R, IL-6, IGF, IGFR1, IL-7, IL-8, IL-10, CD11a, CD58, and antibodies binding to any of their ligands.
706. The cell or the population of cells for use according to any one of embodiments 690-691 and 694-705, the composition for use according to any one of embodiments 690-691 and 694-705, or the method according to any one of embodiments 690-691 and 694-705, wherein the one or more immunosuppressive agents are or have been administered to the subject prior to administration of the cell, the population of cells, or the composition.
707. The cell or the population of cells for use according to any one of embodiments 690-691 and 694-706, the composition for use according to any one of embodiments 690-691 and 694-706, or the method according to any one of embodiments 690-691 and 694-706, wherein the one or more immunosuppressive agents are or have been administered to the subject at least 1, 2, 3, 4, 5, 6, 7, 8, 9, 10, 11, 12, 13, or 14 days prior to administration of the cell, the population of cells, or the composition.
708. The cell or the population of cells for use according to any one of embodiments 690-691 and 694-707, the composition for use according to any one of embodiments 690-691 and 694-707, or the method according to any one of embodiments 690-691 and 694-707, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks or more prior to administration of the cell, the population of cells, or the composition.
709. The cell or the population of cells for use according to any one of embodiments 690-691 and 694-705, the composition for use according to any one of embodiments 690-691 and 694-705, or the method according to any one of embodiments 690-691 and 694-705, wherein the one or more immunosuppressive agents are or have been administered to the subject after administration of the cell, the population of cells, or the composition.
710. The cell or the population of cells for use according to any one of embodiments 690-691 and 694-705and 709, the composition for use according to any one of embodiments 690-691 and 694-705and 709, or the method according to any one of embodiments 690-691 and 694-705and 709, wherein the one or more immunosuppressive agents are or have been administered to the subject at least 1, 2, 3, 4, 5, 6, 7, 8, 9, 10, 11, 12, 13, or 14 days after administration of the cell, the population of cells, or the composition.
711. The cell or the population of cells for use according to any one of embodiments 690-691 and 694-705, 709 and 710, the composition for use according to any one of embodiments 690-691 and 694-705, 709 and 710, or the method according to any one of embodiments 690-691 and 694-705. 709 and 710, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks, or more, after administration of the cell, the population of cells, or the composition.
712. The cell or the population of cells for use according to any one of embodiments 690-691 and 694-705, the composition for use according to any one of embodiments 690-691 and 694-705, or the method according to any one of embodiments 690-691 and 694-705, wherein the one or more immunosuppressive agents are or have been administered to the subject on the same day as the first administration of the cell, the population of cells, or the composition.
713. The cell or the population of cells for use according to any one of embodiments 690-691 and 694-705, the composition for use according to any one of embodiments 690-691 and 694-705, or the method according to any one of embodiments 690-691 and 694-705, wherein the one or more immunosuppressive agents are or have been administered to the subject after administration of a first and/or second administration of the cell, the population of cells, or the composition.
714. The cell or the population of cells for use according to any one of embodiments 690-691 and 694-705, the composition for use according to any one of embodiments 690-691 and 694-705, or the method according to any one of embodiments 690-691 and 694-705, wherein the one or more immunosuppressive agents are or have been administered to the subject prior to administration of a first and/or second administration of the cell, the population of cells, or the composition.
715. The cell or the population of cells for use according to any one of embodiments 690-691 and 694-705, the composition for use according to any one of embodiments 690-691 and 694-705, or the method according to any one of embodiments 690-691 and 694-705, wherein the one or more immunosuppressive agents are or have been administered to the subject at least 1, 2, 3, 4, 5, 6, 7, 8, 9, 10, 11, 12, 13, or 14 days prior to administration of a first and/or second administration of the cell, the population of cells, or the composition.
716. The cell or the population of cells for use according to any one of embodiments 690-691 and 694-705, the composition for use according to any one of embodiments 690-691 and 694-705, or the method according to any one of embodiments 690-691 and 694-705, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks or more prior to administration of a first and/or second administration of the cell, the population of cells, or the composition.
717. The cell or the population of cells for use according to any one of embodiments 690-691 and 694-705, the composition for use according to any one of embodiments 690-691 and 694-705, or the method according to any one of embodiments 690-691 and 694-705, wherein the one or more immunosuppressive agents are or have been administered to the subject at least 1, 2, 3, 4, 5, 6, 7, 8, 9, 10, 11, 12, 13, or 14 days after administration of a first and/or second administration of the cell, the population of cells, or the composition.
718. The cell or the population of cells for use according to any one of embodiments 690-691 and 694-705, the composition for use according to any one of embodiments 690-691 and 694-705, or the method according to any one of embodiments 690-691 and 694-705, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks, or more, after administration of a first and/or second administration of the cell, the population of cells, or the composition.
719. The cell or the population of cells for use according to any one of embodiments 690-691 and 694-718, the composition for use according to any one of embodiments 690-691 and 694-718, or the method according to any one of embodiments 690-691 and 694-718, wherein the one or more immunosuppressive agents are administered at a lower dosage as compared to the dosage administered to reduce immune rejection of a cell that does not comprise the one or more modifications of the cell or the population of cells.
720. The cell or the population of cells for use according to any one of embodiments 648, 650-653 and 657-719, the composition for use according to any one of embodiments 649-653 and 657-719, or the method according to any one of embodiments 654-719, wherein the method further comprises activating the safety switch to induce controlled cell death after the administration of the cell, the population of cells, or the composition to the subject.
721. The cell or the population of cells for use according to any one of embodiments 648, 650-653 and 657-720, the composition for use according to any one of embodiments 649-653 and 657-720, or the method according to any one of embodiments 654-720, wherein the suicide gene or the suicide switch is activated to induce controlled cell death after the administration of the one or more immunosuppressive agents to the subject.
722. The cell or the population of cells for use according to any one of embodiments 648. 650-653 and 657-720, the composition for use according to any one of embodiments 649-653 and 657-720, or the method according to any one of embodiments 654-720, wherein the suicide gene or the suicide switch is activated to induce controlled cell death prior to the administration of the one or more immunosuppressive agents to the subject.
723. The cell or the population of cells for use according to any one of embodiments 648, 650-653 and 657-722, the composition for use according to any one of embodiments 649-653 and 657-722, or the method according to any one of embodiments 654-722, wherein the safety switch is activated to induce controlled cell death in the event of cytotoxicity or other negative consequences to the subject.
724. The cell or the population of cells for use according to any one of embodiments 648, 650-653 and 657-723, the composition for use according to any one of embodiments 649-653 and 657-723, or the method according to any one of embodiments 654-723, wherein the safety switch is an “uncloaking” system wherein upon activation, cells downregulate expression of immunosuppressive factors and/or upregulate expression of immune signaling molecules thereby marking the cell for elimination by the host immune system.
725. The cell or the population of cells for use according to any one of embodiments 648, 650-653 and 657-724, the composition for use according to any one of embodiments 649-653 and 657-724, or the method according to any one of embodiments 654-724, wherein the method comprises administering an agent that allows for depletion of the cell, the population of cells, or the composition.
726. The cell or the population of cells for use according to embodiment 725, the composition for use according to embodiment 725, or the method according to embodiment 725, wherein the agent that allows for depletion of the cell is an antibody that recognizes a protein expressed on the cell surface.
727. The cell or the population of cells for use according to embodiment 726, the composition for use according to embodiment 726, or the method according to embodiment 726, wherein the antibody is selected from the group consisting of an antibody that recognizes CCR4, CD16, CD19, CD20, CD30, EGFR, GD2, HER1, HER2, MUC1, PSMA, and RQR8.
728. The cell or the population of cells for use according to embodiment 726 or embodiment 727, the composition for use according to embodiment 726 or embodiment 727, or the method according to embodiment 726 or embodiment 727, wherein the antibody is selected from the group consisting of mogamulizumab, AFM13, MOR208, obinutuzumab, ublituximab, ocaratuzumab, rituximab, rituximab-Rllb, tomuzotuximab, R05083945 (GA201), cetuximab, Hul4.18K322A, Hul4.18-IL2, Hu3F8, dinituximab, c.60C3-Rllc, and biosimilars thereof.
729. The cell or the population of cells for use according to any one of embodiments 648, 650-653 and 657-726, the composition for use according to any one of embodiments 649-653 and 657-726, or the method according to any one of embodiments 654-726, wherein the method comprises administering an agent that recognizes the one or more tolerogenic factors or the one or more additional tolerogenic factors on the cell surface.
730. The cell or the population of cells for use according to any one of embodiments 648, 650-653 and 657-729, the composition for use according to any one of embodiments 649-653 and 657-729, or the method according to any one of embodiments 654-729, wherein the method further comprises administering one or more additional therapeutic agents to the subject.
731. The cell or the population of cells for use according to any one of embodiments 648, 650-653 and 657-729, the composition for use according to any one of embodiments 649-653 and 657-729, or the method according to any one of embodiments 654-729, wherein the subject has been administered one or more additional therapeutic agents.
732. The cell or the population of cells for use according to any one of embodiments 648, 650-653 and 657-731, the composition for use according to any one of embodiments 649-653 and 657-731, or the method according to any one of embodiments 654-731, wherein the method further comprises monitoring the therapeutic efficacy of the method.
733. The cell or the population of cells for use according to any one of embodiments 648, 650-653 and 657-732, the composition for use according to any one of embodiments 649-653 and 657-732, or the method according to any one of embodiments 654-732, further comprising monitoring the prophylactic efficacy of the method.
734. The cell or the population of cells for use according to embodiment 732 or embodiment 733, the composition for use according to embodiment 732 or embodiment 733, or the method according to embodiment 732 or embodiment 733, wherein the method is repeated until a desired suppression of one or more disease symptoms occurs.
735. A computer-implemented method for profiling the donor capability of a cell or a population of cells for cell therapy, the method comprising:
receiving, by one or more processors, data corresponding to a cell or a population of cells obtained from a donor:
   evaluating, by the one or more processors, the received data for predicted function; and
   profiling, by the one or more processors, a suitability of the cell or a suitability of the population of cells for donor capability as a population of cells for cell therapy.
736. The computer-implemented method of embodiment 735, wherein:
   the evaluating is performed according to any evaluation method of any of embodiments 1-538;
   the profiling is performed according to any identifying method of any of embodiments 1-538; and/or
   the cells or the population of cells are as defined in any of embodiments 1-538.
737. A computer-implemented method comprising the method of any of embodiments 1-538.
738. A computer program comprising instructions which, when the program is executed by a computer, cause the computer to carry out the method of any of embodiments 735-737.
739. A computer-readable medium comprising instructions which, when executed by a computer, cause the computer to carry out the method of any of embodiments 735-737.
740. A data processing device comprising means for carrying out the method of any of embodiments 735-737.
741. The data processing device of embodiment 740, wherein the device is a cloud service.
742. A system comprising a local data processing device and a remote data processing device, wherein: the local data processing device is operable to measure data corresponding to a cell or a population of cells and transmit the data to the remote data processing device; and the remote data processing device is operable to perform the method of any of embodiments 735-737.
743. A method comprising:
classifying a donor capability for cell therapy of a cell or a population of cells of a sample, comprising:
providing a cell or a population of cells obtained from the sample;
assaying a cell function parameter of the cell or the population of cells from the sample;
receiving, at one or more processors, test data for the sample, wherein the test data comprises an assay readout for the cell or the population of cells from the sample;
inputting, using the one or more processors, the test data into a cell function model, wherein the cell function model is configured to classify the sample, based on the test data, as good donor capability-like, or not good donor capability-like; and
classifying, by the one or more processors using the cell function model, the sample as good donor capability-like, or not good donor capability-like.
744. A method comprising:
scoring a donor capability for cell therapy of a cell or a population of cells of a sample, comprising:
providing a cell or a population of cells obtained from the sample;
assaying a cell function parameter of the cell or the population of cells from the sample;
receiving, at one or more processors, test data for the sample, wherein the test data comprises an assay readout for the cell or the population of cells from the sample;
inputting, using the one or more processors, the test data into a cell function model, wherein the cell function model is configured to score the sample, based on the test data, with a number corresponding to donor capability; and
scoring, by the one or more processors using the cell function model, the sample with a number corresponding to donor capability.
745. The method of embodiment 743 or embodiment 744, wherein the model is a mathematical model, a statistical model, a grey box model, a blockmodel, a predictive model, and a deterministic model, or a machine learning model and, optionally, the machine learning model comprising a module employing a regression-based model, a regularization-based model, an instance-based mode, a Bayesian-based model, a clustering-based model, an ensemble-based model, or a neural-network-based model.
746. The method of embodiment 743 or embodiment 744, wherein the cell function model is a machine-learning model trained using assay readout data comprising assay readouts from a plurality of cell samples and cell function data comprising cell function data from a plurality of cell samples.
747. The method of any of embodiments 743-746, wherein the assaying the cell function parameter is according to any of the previous embodiments.
748. A method for classifying a donor capability of a cell or a population of cells for cell therapy, the method comprising:
receiving, at one or more processors, test data comprising assay readouts for the cell or the population of cells;
inputting, using the one or more processors, the test data into a cell function model, wherein the cell function model is configured to classify the cell or the population of cells based on the test data as good donor capability-like, or not good donor capability-like; and
classifying. using the one or more processors and the cell function model, the cell or the population of cells as good donor capability-like, or not good donor capability-like.
749. A method for scoring a donor capability of a cell or a population of cells for cell therapy, the method comprising:
receiving, at one or more processors, test data comprising assay readouts for the cell or the population of cells;
inputting, using the one or more processors, the test data into a cell function model, wherein the cell function model is configured to score the cell or the population of cells based on the test data with a donor capability value; and
scoring, using the one or more processors and the cell function model, the cell or the population of cells with a donor capability value.
750. The method of embodiment 748 or embodiment 749, wherein the model is a mathematical model, a statistical model, a grey box model, a blockmodel, a predictive model, and a deterministic model, or a machine learning model and, optionally, the machine learning model comprising a module employing a regression-based model, a regularization-based model, an instance-based mode, a Bayesian-based model, a clustering-based model, an ensemble-based model, or a neural-network-based model.
751. The method of embodiment 748 or embodiment 749, wherein the cell function model is a machine-learning model trained using assay readout data comprising assay readouts from a plurality of cell samples and cell function data comprising cell function data from a plurality of cell samples.
752. The method of any one of embodiments 743-751, further comprising training the cell function model using the assay readout data and the cell function data.
753. A system, comprising:
one or more processors;
a memory; and
one or more programs, wherein the one or more programs are stored in the memory and configured to be executed by the one or more processors, the one or more programs including instructions for implementing a method, comprising:
receiving, at the one or more processors, test data comprising assay readout data for a sample of a cell or a population of cells from a subject;
inputting, using the one or more processors, the test data into a cell function model, wherein the cell function model is configured to classify the sample, based on the test data, as good donor capability-like, or not good donor capability-like; and
classifying, using the one or more processors and the cell function model, the sample as good donor capability-like, or not good donor capability-like.
754. A system, comprising:
one or more processors;
a memory; and
one or more programs, wherein the one or more programs are stored in the memory and configured to be executed by the one or more processors, the one or more programs including instructions for implementing a method, comprising:
receiving, at the one or more processors, test data comprising assay readout data for a sample of a cell or a population of cells from a subject;
inputting, using the one or more processors, the test data into a cell function model, wherein the cell function model is configured to score the sample, based on the test data, with a number value corresponding to donor capability; and
scoring, using the one or more processors and the cell function model, the sample with a number corresponding to donor capability.
755. The system of embodiment 753 or embodiment 754, wherein the model is a mathematical model, a statistical model, a grey box model, a blockmodel, a predictive model, and a deterministic model, or a machine learning model and, optionally, the machine learning model comprising a module employing a regression-based model, a regularization-based model, an instance-based mode, a Bayesian-based model, a clustering-based model, an ensemble-based model, or a neural-network-based model.
756. The system of embodiment 753 or embodiment 754, wherein the cell function model is a machine-learning model trained using assay readout data comprising assay readouts from a plurality of cell samples and cell function data comprising cell function data from a plurality of cell samples.
757. The system of any of embodiments 753-756, wherein the one or more programs further include instructions for training the cell function model using the assay readout data and the cell function data.
758. A non-transitory computer-readable storage medium storing one or more programs, the one or more programs comprising instructions, which when executed by one or more processors of an electronic device, cause the electronic device to implement a method, comprising:
receiving, at the one or more processors, test data comprising at least one assay readout for the cell or the population of cells;
inputting, using the one or more processors, the test data into a cell function model, wherein the cell function model is configured to classify the sample, based on the test data, as good donor capability-like, or not good donor capability-like; and
classifying. using the one or more processors and the cell function model, the sample as good donor capability-like, or not good donor capability-like.
759. A non-transitory computer-readable storage medium storing one or more programs, the one or more programs comprising instructions, which when executed by one or more processors of an electronic device, cause the electronic device to implement a method, comprising:
receiving, at the one or more processors, test data comprising at least one assay readout for the cell or the population of cells;
inputting, using the one or more processors, the test data into a cell function model, wherein the cell function model is configured to score the sample, based on the test data, with a number corresponding to donor capability; and
scoring, using the one or more processors and the cell function model, the sample with a number corresponding to donor capability.
760. The non-transitory computer-readable storage medium of embodiment 758 or embodiment 759, wherein the model is a mathematical model, a statistical model, a grey box model, a blockmodel, a predictive model, and a deterministic model, or a machine learning model and, optionally, the machine learning model comprising a module employing a regression-based model, a regularization-based model, an instance-based mode, a Bayesian-based model, a clustering-based model, an ensemble-based model, or a neural-network-based model.
761. The non-transitory computer-readable storage medium of embodiment 758 or embodiment 759, wherein the cell function model is a machine-learning model trained using assay readout data comprising assay readouts from a plurality of cell samples and cell function data comprising cell function data from a plurality of cell samples.
762. The non-transitory computer-readable storage medium of any of embodiments 758 to 761, wherein the one or more programs further include instructions, which when executed by one or more processors of an electronic device, cause the electronic device to train the cell function model using the assay readout data and the cell function data.
763. A method comprising:
classifying a donor capability for cell therapy for a sample from a subject, comprising:
providing a cell or a population of cells obtained from the sample from a subject;
assaying a cell function parameter, wherein the assaying provides an assay readout as test data; receiving, at one or more processors, test data for the sample, wherein the test data comprises the assay readout;
inputting, using the one or more processors, the test data into a model, wherein the first cell samples are different from the second cell samples, and wherein the model is configured to classify the sample, based on the test data, as first donor capability-like, or second donor capability-like; and
classifying, by the one or more processors using the model, the sample as first donor capability-like, or second donor capability-like.
764. A method comprising:
scoring a donor capability for cell therapy for a sample from a subject, comprising:
providing a cell or a population of cells obtained from the sample from a subject;
assaying a cell function parameter, wherein the assaying provides an assay readout as test data;
receiving, at one or more processors, test data for the sample, wherein the test data comprises the assay readout;
inputting, using the one or more processors, the test data into a model, wherein the first cell samples are different from the second cell samples, and wherein the model is configured to score the sample, based on the test data, with a number corresponding donor capability; and
scoring, by the one or more processors using the model, the sample with a number corresponding donor capability.
765. The method of embodiment 763 or embodiment 764, wherein the model is a mathematical model, a statistical model, a grey box model, a blockmodel, a predictive model, and a deterministic model, or a machine learning model and, optionally, the machine learning model comprising a module employing a regression-based model, a regularization-based model, an instance-based mode, a Bayesian-based model, a clustering-based model, an ensemble-based model, or a neural-network-based model.
766. The method of embodiment 763 or embodiment 764, wherein the model is a machine-learning model trained using a first assay readout data comprising a first assay readout data from a plurality of first cell samples and a second assay readout data comprising second assay readout data from a plurality of second cell samples.
767. The method of any of embodiments 763-766, wherein the assaying the cell function parameter is according to any of the previous embodiments.
768. A method, comprising:
receiving, at one or more processors, test data for a sample, wherein the test data comprises an assay readout for a cell or a population of cells of the sample;
inputting, using the one or more processors, the test data into a model, wherein the first cell samples are different from the second cell samples, and wherein the model is configured to classify the sample, based on the test data, as first donor capability-like, or second donor capability-like; and
classifying, by the one or more processors using the model, the sample as first donor capability-like, or a second donor capability-like.
769. A method, comprising:
receiving, at one or more processors, test data for a sample, wherein the test data comprises an assay readout for a cell or a population of cells of the sample;
inputting, using the one or more processors, the test data into a model, wherein the first cell samples are different from the second cell samples, and wherein the model is configured to score the sample, based on the test data, with a number corresponding to donor capability; and scoring, by the one or more processors using the model, the sample with a number corresponding to donor capability.
770. The method of embodiment 768 or embodiment 769, wherein the model is a mathematical model, a statistical model, a grey box model, a blockmodel, a predictive model, and a deterministic model, or a machine learning model and, optionally, the machine learning model comprising a module employing a regression-based model, a regularization-based model, an instance-based mode, a Bayesian-based model, a clustering-based model, an ensemble-based model, or a neural-network-based model.
771. The method of embodiment 768 or embodiment 769, wherein the model is a machine-learning model trained using a first assay readout data comprising a first assay readout data from a plurality of first cell samples and a second assay readout data comprising second assay readout data from a plurality of second cell samples.
772. The method, system or non-transitory computer-readable storage medium of any of embodiments 743 to 771 wherein:
the model is trained to classify the cell or population of cells, or the sample, based on the test data, as ambiguous in addition to first donor capability-like or second donor capability-like; and/or
the model is trained to classify the cell or population of cells, or the sample, based on the test data, as ambiguous in addition to as first donor capability-like or second donor capability-like.
773. The method, system or non-transitory computer-readable storage medium of any of embodiments 743 to 772 wherein the model or cell function model is trained to classify the cell or population of cells and/or the sample as one or more additional classification(s) or one or more alternative classification(s).
774. A method of evaluating a cell or a population of cells for predicted function, the method comprising:
   receiving, by one or more processors, input data corresponding to the cell or the population of cells; and
   determining (e.g., inferring), by the one or more processors, a predicted function of the cell or the population of cells using the input data and reference data corresponding to reference cells and/or populations of reference cells (e.g., by comparing the input data and the reference data).
775. A method of predicting in vivo function of a cell or a population of cells, the method comprising:
   receiving, by one or more processors, input data corresponding to the cell or the population of cells; and
   determining (e.g., inferring), by the one or more processors, a predicted in vivo function of the cell or the population of cells using the input data and reference data corresponding to reference cells and/or populations of reference cells [e.g., by comparing the input data and the reference data].
776. A method of training a model to predict function of a cell or a population of cells, the method comprising training, by one or more processors, an algorithm (e.g., in a machine learning module) using reference data corresponding to reference cells and/or populations of reference cells (e.g., obtained using a method in accordance with any one of embodiments 1-538).
777. A method of training a model to predict in vivo cell function of a cell or a population of cells, the method comprising training, by one or more processors, an algorithm (e.g., in a machine learning module) using reference data corresponding to reference cells and/or populations of reference cells.
778. A method of determining one or more cell characteristics (e.g., function(s) and/or parameter(s))(e.g., cell type (or subtype) specific characteristics) indicative of cell(s) being suitable as donor cell(s) for a cell therapy, the method comprising comparing, by one or more processors, reference data generated from reference cells and/or populations of reference cells [e.g., using an algorithm (e.g., in a machine learning module)].
779. The method of any one of embodiments 774-778, wherein:
   (i) the reference data comprises one or more quantitative assay readouts from reference cells and/or the populations of reference cells (e.g., determined using a (e.g., computer-implemented) method in accordance with any one of embodiments 1-538), one or more qualitative assessments of one or more quantitative assay readouts from reference cells and/or the populations of reference cells (e.g., determined using a (e.g., computer-implemented) method in accordance with any preceding embodiment), or a combination thereof,
   (ii) the input data comprises one or more quantitative assay readouts from the cell or the population of cells (e.g., determined using a (e.g., computer-implemented) method in accordance with any one of embodiments 1-538), one or more qualitative assessments of one or more quantitative assay readouts from the cell or the population of cells (e.g., determined using a (e.g., computer-implemented) method in accordance with any one of embodiments 1-538), or a combination thereof, or
   (iii) both (i) and (ii).
780. The method of any one of embodiments 774-779, wherein the input data and/or the reference data comprises data corresponding to age of subject(s) (e.g., from which the cell or the population of cells is derived and/or from which the reference cells and/or the populations of reference cells are derived, respectively), health history [e.g., health status (e.g., current health status)1 of the subject(s), sex of the subject(s), or a combination thereof.
781. The method of any one of embodiments 774-780, wherein the reference data comprises (e.g., further comprises) clinical output data, in vitro experimental data, in vivo experimental data, or a combination thereof (e.g., determined using a (e.g., computer-implemented) method in accordance with any one of embodiments 1-388).
782. The method of any one of embodiments 774-781, wherein the input data and/or the reference data comprises one or more quantitative assay readouts from one or more assays and the one or more assays comprise an in vitro assay, an in vivo assay, an immune assay, a cell activity assay, a cell avidity assay, a cell proliferation assay, a cell cytotoxicity assay, a cellular stress assay, a tumor challenge assay, an expression assay, a cytokine production assay, transcriptomic profiling assay, a proteomic profiling assay, a genomic profiling assay, a genomic stability assay, an epigenetic profiling assay, a cell developmental potential profiling assay, a cell subtyping assay, a cell receptor profiling assay or a combination thereof.
783. The method of embodiment 782, wherein the one or more quantitative assay readouts comprises a cell functionality score, a cell polyfunctionality index, a cell multifunctionality index, an in vivo efficacy score, an in vivo activity score, an in vivo response score, an in vitro efficacy score, an in vitro activity score, an in vitro response score, an immune efficacy score, an immune activity score, an immune response score, a cell activity score, a cell activity response score, a cell specificity score, a cell sensitivity score, a cell avidity score, a cell proliferation score, a cell proliferative index, a cell cytotoxicity score, a cell cytotoxicity response score, a cell stress score, a cell stress response score, a tumor challenge efficacy score, a tumor challenge activity score, a tumor challenge response score, a tumor challenge specificity score, a tumor challenge sensitivity score, an expression profile, an expression signature, an expression signal, an expression score, a bulk cytokine or chemokine production profile, a bulk cytokine or chemokine production signature, a bulk cytokine or chemokine production profile, a bulk cytokine or chemokine production signal, a bulk cytokine or chemokine production score, a single cell cytokine or chemokine production profile, a single cell cytokine or chemokine production signature, a single cell cytokine or chemokine production profile, a single cell cytokine or chemokine production signal, a single cell cytokine or chemokine production score, a transcriptomic profile, a transcriptomic signature, a transcriptomic signature, a transcriptomic score, a pathway profile, a pathway signature, a pathway signal, a pathway score, a proteomic profile, a proteomic signature, a proteomic signal, a proteomic score, a genomic profile, a genomic signature, a genomic signal, a genomic score, a genomic stability profile, a genomic stability signature, a genomic stability signal, a genomic stability score, an epigenetic profile, an epigenetic signature, an epigenetic signal, an epigenetic score, a cell developmental potential assessment, a cell developmental potential profile, a cell developmental potential signature, a cell developmental potential score, a cell subtyping profile, a cell subtyping signature, a cell subtyping score, a cell receptor profile, a cell receptor signature, a cell receptor signal, a cell receptor score, or a combination thereof.
784. The method of any one of embodiments 774-783, wherein the input data and/or the reference data comprises data corresponding to (i) one or more assay readouts, optionally cell activation, cell polyfunctionality, cell multifunctionality, cell cytotoxicity, cell growth rate, one or more characteristics associated with particular cell type, cellular activity associated with the cell or the population of cells, cell cytokine production, or a combination thereof, and/or (ii) data relating to function (e.g., predicted function), optionally durability of cell growth, durability of cell response, cellular ability to elicit adaptive and innate immune responses, or a combination thereof; and optionally cell safety attributes; and/or cell impurity level(s).
785. The method of embodiment 784, wherein the input data and/or the reference data comprises one or more assay readouts and/or data relating to function (e.g., predicted function) from one or more assessments of the cells or the populations of cells or the reference cells or the reference populations of cells, optionally at one or more time points.
786. The method of any one of embodiments 774-785, wherein the one or more qualitative assessments of one or more quantitative assay readouts are qualitative categorizations (e.g., poor, good, usable, or exceptional) (e.g., determined using a (e.g., computer-implemented) method in accordance with any one of embodiments 1-538).
787. The method of any one of embodiments 774-786, wherein the reference data comprises persistence data, engraftment data, durability (e.g., of cell response) data, potency data, hypoimmunogenicity data, or a combination thereof (e.g., determined using a (e.g., computer-implemented) method in accordance with any one of embodiments 1-538).
788. The method of any one of embodiments 774-787, wherein the reference cells and/or the populations of reference cells comprise suitable donor cells and/or unsuitable donor cells (e.g., determined using a(e.g., computer-implemented) method in accordance with any one of embodiments 1-538).
789. The method of any one of embodiments 774-788, wherein the cell or the population of cells comprises a cell in accordance with any one of embodiments 1-538.
790. The method of any one of embodiments 774-789, wherein the reference cells and/or populations of reference cells do not have any modification.
791. The method of any one of embodiments 774-790, wherein the reference cells and/or populations of reference cells have been modified (e.g., in accordance with any one of embodiments 1-538).
792. The method of any one of embodiments 774-791, wherein (i) the input data comprise data corresponding to at least one cell parameter and/or at least one function of the cell or the population of cells, (ii) the reference data comprise data corresponding to at least one cell parameter and/or at least one function of the reference cells and/or the populations of reference cells, or (iii) both (i) and (ii).
793. The method of any one of embodiments 774-792, wherein:
   (i) the input data comprises one or more quantitative assay readouts from the cell or the population of cells and the one or more quantitative assay readouts for the cell or the population of cells comprises one or more single values (e.g., one or more numerical values), one or more complex values (e.g., comprising a plurality of values) (e.g., comprising a range of values) [e.g., comprising multiple separate numerical values (e.g., pixel data from one or more images and/or flow plot data (e.g., from a gel))], one or more qualitative or semi-quantitative values [e.g., comprising one or more non-numerical values (e.g., a qualitative scale)], or a combination thereof (e.g., determined using a (e.g., computer-implemented) method in accordance with any one of embodiments 1-538),
   (ii) the reference data comprises one or more quantitative assay readouts from the reference cells and/or the populations of reference cells and the one or more quantitative assay readouts for the reference cells and/or the populations of reference cells comprises one or more single values (e.g., one or more numerical values), one or more complex values (e.g., comprising a plurality of values) (e.g., comprising a range of values) [e.g., comprising multiple separate numerical values (e.g., pixel data from one or more images and/or flow plot data (e.g., from a gel))], one or more qualitative or semi-quantitative values [e.g., comprising one or more non-numerical values (e.g., a qualitative scale)], or a combination thereof (e.g., determined using a (e.g., computer-implemented) method in accordance with any one of embodiments 1-538), or
   (iii) both (i) and (ii).
794. The method of embodiment 793, wherein the one or more quantitative assay readouts comprises the complex values and/or the qualitative or semi-quantitative values and the complex values and/or the qualitative or semi-quantitative values comprises pixel information from one or more images (e.g., wherein the one or more quantitative assay readouts comprises one or more images from one or more assays), flow plot data (e.g., wherein the one or more quantitative assay readouts comprises one or more images from one or more assays), or a combination thereof (e.g., determined using a (e.g., computer-implemented) method in accordance with any one of embodiments 1-538).
795. The method of embodiment 793 or embodiment 794, wherein the input data and/or the reference data comprises one or more single values that have been converted from one or more complex values and/or qualitative or semi-quantitative values of one or more quantitative assay readouts (e.g., from the cell or the population of cells and/or from the reference cells and/or the populations of reference cells) (e.g., prior to determining the predicted function) [e.g., using a predetermined conversion scheme (e.g., scale) (e.g., by combining (e.g., concatenating) and/or statistically processing (e.g., averaging), by the one or more processors, the values)].
796. The method of embodiment 793 or embodiment 794, wherein the one or more quantitative assay readouts (e.g., from the cell or the population of cells and/or from the reference cells and/or the populations of reference cells) comprise the complex values and/or the qualitative or semi-quantitative values and the method comprises converting, by the one or more processors, the complex values and/or the qualitative or semi-quantitative values to a single value for each of the one or more quantitative assay readouts (e.g., prior to determining the predicted function) [e.g., using a predetermined conversion scheme (e.g., scale) (e.g., by combining (e.g., concatenating) and/or statistically processing (e.g., averaging), by the one or more processors, the values)].
797. The method of any one of embodiments 793-796, wherein determining the predicted function comprises comparing, by the one or more processors, one or more single values for one or more quantitative assay readouts for the cell or the population of cells and one or more single values for one or more quantitative assay readouts for the reference cells and/or the populations of reference cells (e.g., after converting, by the one or more processors, to the single value(s) from one or more complex values and/or one or more qualitative and/or semi-quantitative values).
798. The method of any one of embodiments 793-797, comprising converting, by the one or more processors, one or more quantitative assay readouts into a suitable format for performing the determining (e.g., for input into a machine learning module) [e.g., suitable data format and/or size (e.g., string and/or number format and/or size)].
799. The method of any one of embodiments 793-798, wherein determining the predicted function comprises using one or more weights to weight the input data and/or the reference data (e.g., relative to each other and/or to differently weight different portions of the input data and/or to differently weight different portions of the reference data) (e.g., used in a loss function used in a machine learning module) [e.g., that have been determined by a trained algorithm (e.g., in a machine learning module)].
800. The method of embodiment 799, comprising determining, by the one or more processors, the one or more weights.
801. The method of embodiment 799 or embodiment 800, wherein the one or more weights are for weighting one or more quantitative assay readouts comprised in the input data and/or the reference data.
802. The method of any one of embodiments 799-801, wherein determining the one or more weights comprises receiving, by the one or more processors, user input corresponding to the one or more weights (e.g., via one or more graphical user interfaces (GUIs)).
803. The method of embodiment 802, wherein the user input comprises one or more of the one or more weights and/or one or more cell characteristics (e.g., function(s) and/or parameter(s)) to weight.
804. The method of any one of embodiments 799-803, wherein determining the one or more weights comprises determining the one or more weights using a trained algorithm or training an algorithm (e.g., in a machine learning module).
805. The method of any one of embodiments 709-804, wherein the one or more weights correspond to (e.g., wherein the user input represents):
   (i) one or more cell characteristics to consider or not consider (e.g., one or more quantitative assay readouts to consider or not consider),
   (ii) a preference of one or more cell characteristics over one or more other cell characteristics [e.g., a ranked list of two or more cell characteristics (e.g., a ranked list of some but not all or all available cell characteristics to choose from)](e.g., a preference of one or more quantitative assay readouts over one or more other assay readouts),
   (iii) a selection of one or more subsets of the input data and/or the reference data to weight more heavily or less heavily (e.g., comprising one or more desired weights [e.g., that is/are then fixed or that serves as an initial value that is/are then modified (e.g., through iteration in a machine learning module)], or
   (iv) a combination thereof.
806. The method of any one of embodiments 709-805, comprising comparing, by the one or more processors, the input data and (e.g., to) the reference data (e.g., using a machine learning module).
807. The method of embodiment 806, wherein comparing the input data and the reference data comprises performing, by the one or more processors, a multiparametric comparison between the input data and the reference data.
808. The method of embodiment 807, comprising comparing (e.g., optimizing), by the one or more processors, two or more, three or more, four or more, five or more, six or more, seven or more, eight or more, nine or more, or ten or more parameters.
809. The method of embodiment 807 or embodiment 808, wherein the multiparametric comparison is a biomarker.
810. The method of any one of embodiments 774-809-640, wherein the predicted function comprises one or more of functions selected from the group consisting of durability of cell growth, durability of cell response, and cellular ability to elicit adaptive and innate immune responses.
811. The method of any one of embodiments 774-810, comprising determining (e.g., classifying and/or inferring), by the one or more processors, (e.g., wherein a machine learning module that determines the predicted function outputs) a categorization of the cell or the population of cells that is indicative of the predicted function of the cell or the population of cells.
812. The method of any one of embodiments 774-811, wherein the predicted function comprises a categorization of the cell or the population of cells.
813. The method of embodiment 811 or embodiment 812, wherein the categorization is a qualitative categorization (e.g., that uses a qualitative scale comprising one or more of: ‘poor,’ ‘usable,’ ‘good,’ and ‘exceptional’).
814. The method of embodiment 811 or embodiment 812, wherein the categorization uses a quantitative scale (e.g., provides a numerical value on a predetermined scale).
815. The method of any one of embodiments 812-814, wherein the categorization is indicative of a suitability of the cell or the population of cells for use as donor cell(s) in a cell therapy.
816. The method of any one of embodiments 774-815, determining (e.g., classifying and/or inferring), by the one or more processors, suitability of the cell or the population of cells for use as donor cell(s) in a cell therapy (e.g., wherein the predicted function is indicative of the suitability) (e.g., wherein the suitability is determined using the predicted function) (e.g., using the input data and the reference data in a (e.g., computer-implemented) method to determine suitability in accordance with any one of embodiments 1-538).
817. The method of embodiment 816, wherein determining the suitability comprises determining (e.g., classifying and/or inferring), by the one or more processors, that the cell or the population of cells is/are suitable for use as donor cell(s) (e.g., by a (e.g., computer-implemented) method in accordance with any one of embodiments 1-538).
818. The method of embodiment 816 or embodiment 817, wherein the input data comprises data corresponding to one or more cell characteristics (e.g., function(s) and/or parameter(s)) and the method comprises determining (e.g., classifying and/or inferring), by the one or more processors that the cell or the population of cells is/are suitable for use as donor cell(s) due, in part, to presence and/or absence of one or more traits from the one or more cell characteristics (e.g., by a (e.g., computer-implemented) method in accordance with any one of embodiments 1-538).
819. The method of embodiment 818, wherein the one or more cell characteristics correspond to age of subject from which the cell or the population of cells is derived, health history [e.g., health status (e.g., current health status)] of the subject, sex of the subject, or a combination thereof [e.g., and the one or more traits correspond to an acceptable and/or desirable (e.g., preferred) age or age range, acceptable and/or desirable (e.g., preferred) sex, presence of one or more acceptable and/or desirable (e.g., preferred) health traits, absence of one or more unacceptable and/or undesirable (e.g., non-preferred) health traits, or a combination thereof].
820. The method of embodiment 818 or embodiment 819, comprising determining (e.g., classifying and/or inferring), by the one or more processors, that the cell or the population of cells is suitable due to the presence and/or absence of the one or more traits when the cell or the population of cells would otherwise be less suitable or unsuitable without and/or with having the one or more traits, respectively (e.g., by a (e.g., computer-implemented) method in accordance with any one of embodiments 1-538).
821. The method of any one of embodiments 816-820, wherein determining the suitability comprises using one or more weights to weight the input data and/or the reference data (e.g., relative to each other and/or to differently weight different portions of the input data and/or to differently weight different portions of the reference data) (e.g., used in a loss function used in a machine learning module).
822. The method of embodiment 821, comprising determining, by the one or more processors, one or more weights for use in determining the suitability (e.g., used in a loss function used in a machine learning module) [e.g., wherein determining the one or more weights comprises (i) determining the one or more weights using a trained algorithm or (ii) training an algorithm (e.g., in a machine learning module)].
823. The method of embodiment 821 or embodiment 822, wherein the one or more weights correspond to one or more desirable cell characteristics and/or the one or more weights have been selected (e.g., by user input) to more heavily weight one or more desirable cell characteristics than one or more other cell characteristics (e.g., wherein the one or more desirable cell characteristics are or have been determined, by the one or more processors, using reference data that comprises cell performance data).
824. The method of any one of embodiments 821-823, wherein the one or more weights are for weighting one or more quantitative assay readouts comprised in the input data and/or the reference data.
825. The method of any one of embodiments 821-824, wherein determining the one or more weights comprises receiving, by the one or more processors, user input corresponding to the one or more weights (e.g., via one or more graphical user interfaces (GUIs)).
826. The method of embodiment 825, wherein the user input comprises one or more of the one or more weights and/or one or more cell characteristics to weight.
827. The method of any one of embodiments 821-826, wherein the one or more weights correspond to (e.g., wherein the user input represents):
   (i) one or more cell characteristics to consider or not consider (e.g., one or more quantitative assay readouts to consider or not consider),
   (ii) a preference of one or more cell characteristics over one or more other cell characteristics [e.g., a ranked list of two or more cell characteristics (e.g., a ranked list of some but not all or all available cell characteristics to choose from)](e.g., a preference of one or more quantitative assay readouts over one or more other assay readouts),
   (iii) a selection of one or more subsets of the input data and/or the reference data to weight more heavily or less heavily (e.g., comprising one or more desired weights [e.g., that is/are then fixed or that serves as an initial value that is/are then modified (e.g., through iteration in a machine learning module)], or
   (iv) a combination thereof.
828. The method of any one of embodiments 774-827, wherein the method is performed using (e.g., wherein the predicted function of the cell or the population of cells is determined using) a machine learning module that (a) has been trained using the reference data and (b) receives the input data as an input [e.g., in addition to one or more other inputs (e.g., user-selected weight(s) to be applied (e.g., in a loss function))].
829. The method of embodiment 828, wherein the machine learning module employs a regression-based model (e.g., a logistic regression model), a regularization-based model (e.g., an elastic net model or a ridge regression model), an instance-based model (e.g., a support vector machine or a k-nearest neighbor model), a Bayesian-based model (e.g., a naive-based model or a Gaussian naive-based model), a clustering-based model (e.g., an expectation maximization model), an ensemble-based model (e.g., an adaptive boosting model, a random forest model, a bootstrap-aggregation model, or a gradient boosting machine model), or a neural-network-based model (e.g., a convolutional neural network, a recurrent neural network, autoencoder, a back propagation network, or a stochastic gradient descent network).
830. A system comprising (e.g., the) one or more processors: a memory; one or more programs; and optionally a machine learning module (e.g., stored in the memory), wherein the one or more programs are stored in the memory and are executable by the one or more processors, the one or more programs comprising instructions for implementing the method according to any one of embodiments 774-829.
831. The method of any one of embodiments 735-830, comprising performing one or more assays and/or one or more qualitative assessments of one or more quantitative assay readouts (e.g., in a method according to any one of embodiments 1-538) to generate the input data [e.g., that is then converted to a different size and/or format (e.g., a single value) (e.g., prior to being input into a machine learning module)].
832. The method of embodiment 831, wherein performing the one or more assays comprises obtaining the cell or the population of cells, assaying the cell or the population of cells for at least one cell parameter, and obtaining at least one assay readout.
833. The method of embodiment 831 or 832, wherein the one or more assays assay for 2 or more, 3 or more, four or more, five or more, six or more, seven or more, eight or more, nine or more, or at least ten cell parameters.
834. A method comprising making a cell therapy using a suitable cell or population of cells as donor cell(s) (e.g., in a method according to any one of embodiments 1-538), wherein the cell or the population of cells have been determined to be suitable by a method according to any one of embodiments 735-832.
835. A method comprising administering a cell therapy to a subject (e.g., in a method according to any one of embodiments 1-538), wherein the cell therapy comprises one or more donor cells that have been determined to be suitable by a method according to any one of embodiments 735-834.
836. The method of any one of embodiments 735-835, further comprising introducing into the cell or the population of cells one or more modifications (e.g., in accordance with any one of embodiments 1-538).
837. A system for evaluating a cell or a population of cells for predicted function, the system comprising one or more processors, a memory; and one or more programs, wherein the one or more programs are stored in the memory and are executable by the one or more processors, the one or more programs comprising instructions for implementing a method comprising:
   receiving, by one or more processors, input data corresponding to the cell or the population of cells; and
   determining (e.g., inferring), by the one or more processors, a predicted function of the cell or the population of cells using the input data and reference data corresponding to reference cells and/or populations of reference cells (e.g., by comparing the input data and the reference data).
838. A system for predicting in vivo function of a cell or a population of cells, the system comprising one or more processors; a memory; and one or more programs, wherein the one or more programs are stored in the memory and are executable by the one or more processors, the one or more programs comprising instructions for implementing a method comprising:
   receiving, by one or more processors, input data corresponding to the cell or the population of cells; and
   determining (e.g., inferring), by the one or more processors, a predicted in vivo function of the cell or the population of cells using the input data and reference data corresponding to reference cells and/or populations of reference cells [e.g., by comparing the input data and the reference data].
839. A system for training a model to predict function of a cell or a population of cells, the system comprising one or more processors; a memory; and one or more programs, wherein the one or more programs are stored in the memory and are executable by the one or more processors, the one or more programs comprising instructions for implementing a method comprising training, by one or more processors, an algorithm (e.g., in a machine learning module) using reference data corresponding to reference cells and/or populations of reference cells (e.g., obtained using a method in accordance with any one of embodiments 1-538).
840. A system for training a model to predict in vivo cell function of a cell or a population of cells, the system comprising one or more processors: a memory; and one or more programs, wherein the one or more programs are stored in the memory and are executable by the one or more processors, the one or more programs comprising instructions for implementing a method comprising training, by one or more processors, an algorithm(e.g., in a machine learning module) using reference data corresponding to reference cells and/or populations of reference cells.
841. A system for determining one or more cell characteristics (e.g., function(s) and/or parameter(s)) (e.g., cell type (or subtype) specific characteristics) indicative of cell(s) being suitable as donor cell(s) for a cell therapy, the system comprising one or more processors; a memory; and one or more programs, wherein the one or more programs are stored in the memory and are executable by the one or more processors, the one or more programs comprising instructions for implementing a method comprising comparing, by one or more processors, reference data generated from reference cells and/or populations of reference cells [e.g., using an algorithm (e.g., in a machine learning module)].
842. The system of any one of embodiments 837-841, wherein:
   (i) the reference data comprises one or more quantitative assay readouts from reference cells and/or the populations of reference cells (e.g., determined using a (e.g., computer-implemented) method in accordance with any one of embodiments 1-538), one or more qualitative assessments of one or more quantitative assay readouts from reference cells and/or the populations of reference cells (e.g., determined using a (e.g., computer-implemented) method in accordance with any preceding embodiment), or a combination thereof,
   (ii) the input data comprises one or more quantitative assay readouts from the cell or the population of cells (e.g., determined using a (e.g., computer-implemented) method in accordance with any one of embodiments 1-538), one or more qualitative assessments of one or more quantitative assay readouts from the cell or the population of cells (e.g., determined using a (e.g., computer-implemented) method in accordance with any one of embodiments 1-538), or a combination thereof, or
   (iii) both (i) and (ii).
843. The system of any one of embodiments 837-842, wherein the input data and/or the reference data comprises data corresponding to age of subject(s) (e.g., from which the cell or the population of cells is derived and/or from which the reference cells and/or the populations of reference cells are derived, respectively), health history [e.g., health status (e.g., current health status)] of the subject(s), sex of the subject(s), or a combination thereof.
844. The system of any one of embodiments 837-843, wherein the reference data comprises (e.g., further comprises) clinical output data, in vitro experimental data, in vivo experimental data, or a combination thereof (e.g., determined using a (e.g., computer-implemented) method in accordance with any one of embodiments 1-538).
845. The system of any one of embodiments 837-844, wherein the input data and/or the reference data comprises one or more quantitative assay readouts from one or more assays and the one or more assays comprise an in vitro assay, an in vivo assay, an immune assay, a cell activity assay, a cell avidity assay, a cell proliferation assay, a cell cytotoxicity assay, a cellular stress assay, a tumor challenge assay, an expression assay, a cytokine production assay, transcriptomic profiling assay, a proteomic profiling assay, a genomic profiling assay, a genomic stability assay, an epigenetic profiling assay, a cell developmental potential profiling assay, a cell subtyping assay, a cell receptor profiling assay or a combination thereof.
846. The system of embodiment 845, wherein the one or more quantitative assay readouts comprises a cell functionality score, a cell polyfunctionality index, a cell multifunctionality index, an in vivo efficacy score, an in vivo activity score, an in vivo response score, an in vitro efficacy score, an in vitro activity score, an in vitro response score, an immune efficacy score, an immune activity score, an immune response score, a cell activity score, a cell activity response score, a cell specificity score, a cell sensitivity score, a cell avidity score, a cell proliferation score, a cell proliferative index, a cell cytotoxicity score, a cell cytotoxicity response score, a cell stress score, a cell stress response score, a tumor challenge efficacy score, a tumor challenge activity score, a tumor challenge response score, a tumor challenge specificity score, a tumor challenge sensitivity score, an expression profile, an expression signature, an expression signal, an expression score, a bulk cytokine or chemokine production profile, a bulk cytokine or chemokine production signature, a bulk cytokine or chemokine production profile, a bulk cytokine or chemokine production signal, a bulk cytokine or chemokine production score, a single cell cytokine or chemokine production profile, a single cell cytokine or chemokine production signature, a single cell cytokine or chemokine production profile, a single cell cytokine or chemokine production signal, a single cell cytokine or chemokine production score, a transcriptomic profile, a transcriptomic signature, a transcriptomic signature, a transcriptomic score, a pathway profile, a pathway signature, a pathway signal, a pathway score, a proteomic profile, a proteomic signature, a proteomic signal, a proteomic score, a genomic profile, a genomic signature, a genomic signal, a genomic score, a genomic stability profile, a genomic stability signature, a genomic stability signal, a genomic stability score, an epigenetic profile, an epigenetic signature, an epigenetic signal, an epigenetic score, a cell developmental potential assessment, a cell developmental potential profile, a cell developmental potential signature, a cell developmental potential score, a cell subtyping profile, a cell subtyping signature, a cell subtyping score, a cell receptor profile, a cell receptor signature, a cell receptor signal, a cell receptor score, or a combination thereof.
847. The system of any one of embodiments 837-846, wherein the input data and/or the reference data comprises data corresponding to (i) one or more assay readouts, optionally cell activation, cell polyfunctionality, cell multifunctionality, cell cytotoxicity, cell growth rate, one or more characteristics associated with particular cell type, cellular activity associated with the cell or the population of cells, cell cytokine production, or a combination thereof, and/or (ii) data relating to function (e.g., predicted function), optionally durability of cell growth, durability of cell response, cellular ability to elicit adaptive and innate immune responses, or a combination thereof; and optionally cell safety attributes; and/or cell impurity level(s).
848. The system of embodiment 847, wherein the input data and/or the reference data comprises one or more assay readouts and/or data relating to function (e.g., predicted function) from one or more assessments of the cells or the populations of cells or the reference cells or the reference populations of cells, optionally at one or more time points.
849. The system of any one of embodiments 837-848, wherein the one or more qualitative assessments of one or more quantitative assay readouts are qualitative categorizations (e.g., poor, good, usable, or exceptional) (e.g., determined using a (e.g., computer-implemented) method in accordance with any one of embodiments 1-538).
850. The system of any one of embodiments 837-849, wherein the reference data comprises persistence data, engraftment data, durability (e.g., of cell response) data, potency data, hypoimmunogenicity data, or a combination thereof (e.g., determined using a (e.g., computer-implemented) method in accordance with any one of embodiments 1-538).
851. The system of any one of embodiments 837-850, wherein the reference cells and/or the populations of reference cells comprise suitable donor cells and/or unsuitable donor cells (e.g., determined using a(e.g., computer-implemented) method in accordance with any one of embodiments 1-538).
852. The system of any one of embodiments 837-851, wherein the cell or the population of cells comprises a cell in accordance with any one of embodiments 1-538.
853. The system of any one of embodiments 837-852, wherein the reference cells and/or populations of reference cells do not have any modification.
854. The system of any one of embodiments 837-853, wherein the reference cells and/or populations of reference cells have been modified (e.g., in accordance with any one of embodiments 1-538).
855. The system of any one of embodiments 837-854, wherein (i) the input data comprise data corresponding to at least one cell parameter and/or at least one function of the cell or the population of cells, (ii) the reference data comprise data corresponding to at least one cell parameter and/or at least one function of the reference cells and/or the populations of reference cells, or (iii) both (i) and (ii).
856. The system of any one of embodiments 837-855, wherein:
   (i) the input data comprises one or more quantitative assay readouts from the cell or the population of cells and the one or more quantitative assay readouts for the cell or the population of cells comprises one or more single values (e.g., one or more numerical values), one or more complex values (e.g., comprising a plurality of values) (e.g., comprising a range of values) [e.g., comprising multiple separate numerical values (e.g., pixel data from one or more images and/or flow plot data (e.g., from a gel))], one or more qualitative or semi-quantitative values [e.g., comprising one or more non-numerical values (e.g., a qualitative scale)], or a combination thereof (e.g., determined using a (e.g., computer-implemented) method in accordance with any one of embodiments 1-538),
   (ii) the reference data comprises one or more quantitative assay readouts from the reference cells and/or the populations of reference cells and the one or more quantitative assay readouts for the reference cells and/or the populations of reference cells comprises one or more single values (e.g., one or more numerical values), one or more complex values (e.g., comprising a plurality of values) (e.g., comprising a range of values) [e.g., comprising multiple separate numerical values (e.g., pixel data from one or more images and/or flow plot data (e.g., from a gel))], one or more qualitative or semi-quantitative values [e.g., comprising one or more non-numerical values (e.g., a qualitative scale)], or a combination thereof (e.g., determined using a (e.g., computer-implemented) method in accordance with any one of embodiments 1-538), or
   (iii) both (i) and (ii).
857. The system of embodiment 856, wherein the one or more quantitative assay readouts comprises the complex values and/or the qualitative or semi-quantitative values and the complex values and/or the qualitative or semi-quantitative values comprises pixel information from one or more images (e.g., wherein the one or more quantitative assay readouts comprises one or more images from one or more assays), flow plot data (e.g., wherein the one or more quantitative assay readouts comprises one or more images from one or more assays), or a combination thereof (e.g., determined using a (e.g., computer-implemented) method in accordance with any one of embodiments 1-538).
858. The system of embodiment 856 or embodiment 857, wherein the input data and/or the reference data comprises one or more single values that have been converted from one or more complex values and/or qualitative or semi-quantitative values of one or more quantitative assay readouts (e.g., from the cell or the population of cells and/or from the reference cells and/or the populations of reference cells) (e.g., prior to determining the predicted function) [e.g., using a predetermined conversion scheme (e.g., scale) (e.g., by combining (e.g., concatenating) and/or statistically processing (e.g., averaging), by the one or more processors, the values)].
859. The system of embodiment 856 or embodiment 857, wherein the one or more quantitative assay readouts (e.g., from the cell or the population of cells and/or from the reference cells and/or the populations of reference cells) comprise the complex values and/or the qualitative or semi-quantitative values and the method comprises converting, by the one or more processors, the complex values and/or the qualitative or semi-quantitative values to a single value for each of the one or more quantitative assay readouts (e.g., prior to determining the predicted function) [e.g., using a predetermined conversion scheme (e.g., scale) (e.g., by combining (e.g., concatenating) and/or statistically processing (e.g., averaging), by the one or more processors, the values)].
860. The system of any one of embodiments 856-859, wherein determining the predicted function comprises comparing, by the one or more processors, one or more single values for one or more quantitative assay readouts for the cell or the population of cells and one or more single values for one or more quantitative assay readouts for the reference cells and/or the populations of reference cells (e.g., after converting, by the one or more processors, to the single value(s) from one or more complex values and/or one or more qualitative and/or semi-quantitative values).
861. The system of any one of embodiments 837-860, wherein the method comprises converting, by the one or more processors, one or more quantitative assay readouts into a suitable format for performing the determining (e.g., for input into a machine learning module) [e.g., suitable data format and/or size (e.g., string and/or number format and/or size)].
862. The system of any one of embodiments 837-861, wherein determining the predicted function comprises using one or more weights to weight the input data and/or the reference data (e.g., relative to each other and/or to differently weight different portions of the input data and/or to differently weight different portions of the reference data) (e.g., used in a loss function used in a machine learning module) [e.g., that have been determined by a trained algorithm (e.g., in a machine learning module)].
863. The system of embodiment 862, wherein the method comprises determining, by the one or more processors, the one or more weights.
864. The system of embodiment 862 or embodiment 863, wherein the one or more weights are for weighting one or more quantitative assay readouts comprised in the input data and/or the reference data.
865. The system of any one of embodiments 862-864, wherein determining the one or more weights comprises receiving, by the one or more processors, user input corresponding to the one or more weights (e.g., via one or more graphical user interfaces (GUIs)).
866. The system of embodiment 865, wherein the user input comprises one or more of the one or more weights and/or one or more cell characteristics (e.g., function(s) and/or parameter(s)) to weight.
867. The system of any one of embodiments 862-866, wherein determining the one or more weights comprises determining the one or more weights using a trained algorithm or training an algorithm (e.g., in a machine learning module).
868. The system of any one of embodiments 862-867, wherein the one or more weights correspond to (e.g., wherein the user input represents):
   (i) one or more cell characteristics to consider or not consider (e.g., one or more quantitative assay readouts to consider or not consider),
   (ii) a preference of one or more cell characteristics over one or more other cell characteristics [e.g., a ranked list of two or more cell characteristics (e.g., a ranked list of some but not all or all available cell characteristics to choose from)](e.g., a preference of one or more quantitative assay readouts over one or more other assay readouts),
   (iii) a selection of one or more subsets of the input data and/or the reference data to weight more heavily or less heavily (e.g., comprising one or more desired weights [e.g., that is/are then fixed or that serves as an initial value that is/are then modified (e.g., through iteration in a machine learning module)], or
   (iv) a combination thereof.
869. The system of any one of embodiments 862-868, wherein the method comprises comparing, by the one or more processors, the input data and (e.g., to) the reference data (e.g., using a machine learning module).
870. The system of embodiment 869, wherein comparing the input data and the reference data comprises performing, by the one or more processors, a multiparametric comparison between the input data and the reference data.
871. The system of embodiment 870, wherein the method comprises comparing (e.g., optimizing), by the one or more processors, two or more, three or more, four or more, five or more, six or more, seven or more, eight or more, nine or more, or ten or more parameters.
872. The system of embodiment 870 or embodiment 871, wherein the multiparametric comparison is a biomarker.
873. The system of any one of embodiments 862-872, wherein the predicted function comprises one or more of functions selected from the group consisting of durability of cell growth, durability of cell response, and cellular ability to elicit adaptive and innate immune responses.
874. The system of any one of embodiments 862-873, wherein the method comprises determining (e.g., classifying and/or inferring), by the one or more processors, (e.g., wherein a machine learning module that determines the predicted function outputs) a categorization of the cell or the population of cells that is indicative of the predicted function of the cell or the population of cells.
875. The system of any one of embodiments 862-874, wherein the predicted function comprises a categorization of the cell or the population of cells.
876. The system of embodiment 874 or embodiment 875, wherein the categorization is a qualitative categorization (e.g., that uses a qualitative scale comprising one or more of: ‘poor,’ ‘usable,’ ‘good,’ and ‘exceptional’).
877. The system of embodiment 874 or embodiment 875, wherein the categorization uses a quantitative scale (e.g., provides a numerical value on a predetermined scale).
878. The system of any one of embodiments 874-876, wherein the categorization is indicative of a suitability of the cell or the population of cells for use as donor cell(s) in a cell therapy.
879. The system of any one of embodiments 862-879, wherein the method comprises determining (e.g., classifying and/or inferring), by the one or more processors, suitability of the cell or the population of cells for use as donor cell(s) in a cell therapy (e.g., wherein the predicted function is indicative of the suitability) (e.g., wherein the suitability is determined using the predicted function) (e.g., using the input data and the reference data in a (e.g., computer-implemented) method to determine suitability in accordance with any one of embodiments 1-538).
880. The system of embodiment 879, wherein determining the suitability comprises determining (e.g., classifying and/or inferring), by the one or more processors, that the cell or the population of cells is/are suitable for use as donor cell(s) (e.g., by a (e.g., computer-implemented) method in accordance with any one of embodiments 1-538).
881. The system of embodiment 879 or embodiment 880, wherein the input data comprises data corresponding to one or more cell characteristics (e.g., function(s) and/or parameter(s)) and the method comprises determining (e.g., classifying and/or inferring), by the one or more processors that the cell or the population of cells is/are suitable for use as donor cell(s) due, in part, to presence and/or absence of one or more traits from the one or more cell characteristics (e.g., by a (e.g., computer-implemented) method in accordance with any one of embodiments 1-538).
882. The system of embodiment 881, wherein the one or more cell characteristics correspond to age of subject from which the cell or the population of cells is derived, health history [e.g., health status (e.g., current health status)] of the subject, sex of the subject, or a combination thereof [e.g., and the one or more traits correspond to an acceptable and/or desirable (e.g., preferred) age or age range, acceptable and/or desirable (e.g., preferred) sex, presence of one or more acceptable and/or desirable (e.g., preferred) health traits, absence of one or more unacceptable and/or undesirable (e.g., non-preferred) health traits, or a combination thereof].
883. The system of embodiment 881 or embodiment 882, wherein the method comprises determining (e.g., classifying and/or inferring), by the one or more processors, that the cell or the population of cells is suitable due to the presence and/or absence of the one or more traits when the cell or the population of cells would otherwise be less suitable or unsuitable without and/or with having the one or more traits, respectively (e.g., by a (e.g., computer-implemented) method in accordance with any one of embodiments 1-538).
884. The system of any one of embodiments 879-883, wherein determining the suitability comprises using one or more weights to weight the input data and/or the reference data (e.g., relative to each other and/or to differently weight different portions of the input data and/or to differently weight different portions of the reference data) (e.g., used in a loss function used in a machine learning module).
885. The system of embodiment 884, the method comprises determining, by the one or more processors, one or more weights for use in determining the suitability (e.g., used in a loss function used in a machine learning module) [e.g., wherein determining the one or more weights comprises (i) determining the one or more weights using a trained algorithm or (ii) training an algorithm (e.g., in a machine learning module)].
886. The system of embodiment 884 or embodiment 885, wherein the one or more weights correspond to one or more desirable cell characteristics and/or the one or more weights have been selected (e.g., by user input) to more heavily weight one or more desirable cell characteristics than one or more other cell characteristics (e.g., wherein the one or more desirable cell characteristics are or have been determined, by the one or more processors, using reference data that comprises cell performance data).
887. The system of any one of embodiments 884-886, wherein the one or more weights are for weighting one or more quantitative assay readouts comprised in the input data and/or the reference data.
888. The system of any one of embodiments 884-887, wherein determining the one or more weights comprises receiving, by the one or more processors, user input corresponding to the one or more weights (e.g., via one or more graphical user interfaces (GUIs)).
889. The system of embodiment 888, wherein the user input comprises one or more of the one or more weights and/or one or more cell characteristics to weight.
890. The system of any one of embodiments 884-889, wherein the one or more weights correspond to (e.g., wherein the user input represents):
   (i) one or more cell characteristics to consider or not consider (e.g., one or more quantitative assay readouts to consider or not consider),
   (ii) a preference of one or more cell characteristics over one or more other cell characteristics [e.g., a ranked list of two or more cell characteristics (e.g., a ranked list of some but not all or all available cell characteristics to choose from)](e.g., a preference of one or more quantitative assay readouts over one or more other assay readouts),
   (iii) a selection of one or more subsets of the input data and/or the reference data to weight more heavily or less heavily (e.g., comprising one or more desired weights [e.g., that is/are then fixed or that serves as an initial value that is/are then modified (e.g., through iteration in a machine learning module)], or
   (iv) a combination thereof.
891. The system of any one of embodiments 862-890, comprising a machine learning module (e.g., stored in the memory), wherein the method is performed using (e.g., wherein the predicted function of the cell or the population of cells is determined using) the machine learning module that (a)has been trained using the reference data and (b) receives the input data as an input [e.g., in addition to one or more other inputs (e.g., user-selected weight(s) to be applied (e.g., in a loss function))].
892. The system of embodiment 891, wherein the machine learning module employs a regression-based model (e.g., a logistic regression model), a regularization-based model (e.g., an elastic net model or a ridge regression model), an instance-based model (e.g., a support vector machine or a k-nearest neighbor model), a Bayesian-based model (e.g., a naive-based model or a Gaussian naive-based model), a clustering-based model (e.g., an expectation maximization model), an ensemble-based model (e.g., an adaptive boosting model, a random forest model, a bootstrap-aggregation model, or a gradient boosting machine model), or a neural-network-based model (e.g., a convolutional neural network, a recurrent neural network, autoencoder, a back propagation network, or a stochastic gradient descent network).
893. A non-transitory computer-readable storage medium storing one or more programs, the one or more programs comprising instructions, which when executed by one or more processors of a computing device, cause the device to implement a method comprising:
   receiving, by one or more processors, input data corresponding to the cell or the population of cells; and
   determining (e.g., inferring), by the one or more processors, a predicted function of the cell or the population of cells using the input data and reference data corresponding to reference cells and/or populations of reference cells (e.g., by comparing the input data and the reference data).
894. A non-transitory computer-readable storage medium storing one or more programs, the one or more programs comprising instructions, which when executed by one or more processors of a computing device, cause the device to implement a method comprising:
   receiving, by one or more processors, input data corresponding to the cell or the population of cells; and
   determining (e.g., inferring), by the one or more processors, a predicted in vivo function of the cell or the population of cells using the input data and reference data corresponding to reference cells and/or populations of reference cells [e.g., by comparing the input data and the reference data].
895. A non-transitory computer-readable storage medium storing one or more programs, the one or more programs comprising instructions, which when executed by one or more processors of a computing device, cause the device to implement a method comprising training, by one or more processors, an algorithm(e.g., in a machine learning module) using reference data corresponding to reference cells and/or populations of reference cells (e.g., obtained using a method in accordance with any one of embodiments 1-538).
896. A non-transitory computer-readable storage medium storing one or more programs, the one or more programs comprising instructions, which when executed by one or more processors of a computing device, cause the device to implement a method comprising training, by one or more processors, an algorithm(e.g., in a machine learning module)using reference data corresponding to reference cells and/or populations of reference cells.
897. A non-transitory computer-readable storage medium storing one or more programs, the one or more programs comprising instructions, which when executed by one or more processors of a computing device, cause the device to implement a method comprising comparing, by one or more processors, reference data generated from reference cells and/or populations of reference cells [e.g., using an algorithm (e.g., in a machine learning module)].
898. The non-transitory computer-readable storage medium of any one of embodiments 893-897, wherein:
   (i) the reference data comprises one or more quantitative assay readouts from reference cells and/or the populations of reference cells (e.g., determined using a (e.g., computer-implemented) method in accordance with any one of embodiments 1-538), one or more qualitative assessments of one or more quantitative assay readouts from reference cells and/or the populations of reference cells (e.g., determined using a (e.g., computer-implemented) method in accordance with any preceding embodiment), or a combination thereof,
   (ii) the input data comprises one or more quantitative assay readouts from the cell or the population of cells (e.g., determined using a (e.g., computer-implemented) method in accordance with any one of embodiments 1-538), one or more qualitative assessments of one or more quantitative assay readouts from the cell or the population of cells (e.g., determined using a (e.g., computer-implemented) method in accordance with any one of embodiments 1-538), or a combination thereof, or
   (iii) both (i) and (ii).
899. The non-transitory computer-readable storage medium of any one of embodiments 893-898, wherein the input data and/or the reference data comprises data corresponding to age of subject(s) (e.g., from which the cell or the population of cells is derived and/or from which the reference cells and/or the populations of reference cells are derived, respectively), health history [e.g., health status (e.g., current health status)] of the subject(s), sex of the subject(s), or a combination thereof.
900. The non-transitory computer-readable storage medium of any one of embodiments 893-899, wherein the reference data comprises (e.g., further comprises) clinical output data, in vitro experimental data, in vivo experimental data, or a combination thereof (e.g., determined using a (e.g., computer-implemented) method in accordance with any one of embodiments 1-538).
901. The non-transitory computer-readable storage medium of any one of embodiments 724-731, wherein the input data and/or the reference data comprises one or more quantitative assay readouts from one or more assays and the one or more assays comprise an in vitro assay, an in vivo assay, an immune assay, a cell activity assay, a cell avidity assay, a cell proliferation assay, a cell cytotoxicity assay, a cellular stress assay, a tumor challenge assay, an expression assay, a cytokine production assay, transcriptomic profiling assay, a proteomic profiling assay, a genomic profiling assay, a genomic stability assay, an epigenetic profiling assay, a cell developmental potential profiling assay, a cell subtyping assay, a cell receptor profiling assay or a combination thereof.
902. The non-transitory computer-readable storage medium of embodiment 901, wherein the one or more quantitative assay readouts comprises a cell functionality score, a cell polyfunctionality index, a cell multifunctionality index, an in vivo efficacy score, an in vivo activity score, an in vivo response score, an in vitro efficacy score, an in vitro activity score, an in vitro response score, an immune efficacy score, an immune activity score, an immune response score, a cell activity score, a cell activity response score, a cell specificity score, a cell sensitivity score, a cell avidity score, a cell proliferation score, a cell proliferative index, a cell cytotoxicity score, a cell cytotoxicity response score, a cell stress score, a cell stress response score, a tumor challenge efficacy score, a tumor challenge activity score, a tumor challenge response score, a tumor challenge specificity score, a tumor challenge sensitivity score, an expression profile, an expression signature, an expression signal, an expression score, a bulk cytokine or chemokine production profile, a bulk cytokine or chemokine production signature, a bulk cytokine or chemokine production profile, a bulk cytokine or chemokine production signal, a bulk cytokine or chemokine production score, a single cell cytokine or chemokine production profile, a single cell cytokine or chemokine production signature, a single cell cytokine or chemokine production profile, a single cell cytokine or chemokine production signal, a single cell cytokine or chemokine production score, a transcriptomic profile, a transcriptomic signature, a transcriptomic signature, a transcriptomic score, a pathway profile, a pathway signature, a pathway signal, a pathway score, a proteomic profile, a proteomic signature, a proteomic signal, a proteomic score, a genomic profile, a genomic signature, a genomic signal, a genomic score, a genomic stability profile, a genomic stability signature, a genomic stability signal, a genomic stability score, an epigenetic profile, an epigenetic signature, an epigenetic signal, an epigenetic score, a cell developmental potential assessment, a cell developmental potential profile, a cell developmental potential signature, a cell developmental potential score, a cell subtyping profile, a cell subtyping signature, a cell subtyping score, a cell receptor profile, a cell receptor signature, a cell receptor signal, a cell receptor score, or a combination thereof.
903. The non-transitory computer-readable storage medium of any one of embodiments 893-902, wherein the input data and/or the reference data comprises data corresponding to (i) one or more assay readouts, optionally cell activation, cell polyfunctionality, cell multifunctionality, cell cytotoxicity, cell growth rate, one or more characteristics associated with particular cell type, cellular activity associated with the cell or the population of cells, cell cytokine production, or a combination thereof, and/or (ii) data relating to function (e.g., predicted function), optionally durability of cell growth, durability of cell response, cellular ability to elicit adaptive and innate immune responses, or a combination thereof; and optionally cell safety attributes; and/or cell impurity level(s).
904. The non-transitory computer-readable storage medium of embodiment 903, wherein the input data and/or the reference data comprises one or more assay readouts and/or data relating to function (e.g., predicted function) from one or more assessments of the cells or the populations of cells or the reference cells or the reference populations of cells, optionally at one or more time points.
905. The non-transitory computer-readable storage medium of any one of embodiments 893-904, wherein the one or more qualitative assessments of one or more quantitative assay readouts are qualitative categorizations (e.g., poor, good, usable, or exceptional) (e.g., determined using a (e.g., computer-implemented) method in accordance with any one of embodiments 1-538).
906. The non-transitory computer-readable storage medium of any one of embodiments 893-905, wherein the reference data comprises persistence data, engraftment data, durability (e.g., of cell response) data, potency data, hypoimmunogenicity data, or a combination thereof (e.g., determined using a (e.g., computer-implemented) method in accordance with any one of embodiments 1-538).
907. The non-transitory computer-readable storage medium of any one of embodiments 893-906, wherein the reference cells and/or the populations of reference cells comprise suitable donor cells and/or unsuitable donor cells (e.g., determined using a (e.g., computer-implemented) method in accordance with any one of embodiments 1-538).
908. The non-transitory computer-readable storage medium of any one of embodiments 893-907, wherein the cell or the population of cells comprises a cell in accordance with any one of embodiments 1-538.
909. The non-transitory computer-readable storage medium of any one of embodiments 893-908, wherein the reference cells and/or populations of reference cells do not have any modification.
910. The non-transitory computer-readable storage medium of any one of embodiments 893-909, wherein the reference cells and/or populations of reference cells have been modified (e.g., in accordance with any one of embodiments 1-538).
911. The non-transitory computer-readable storage medium of any one of embodiments 893-910, wherein (i) the input data comprise data corresponding to at least one cell parameter and/or at least one function of the cell or the population of cells, (ii) the reference data comprise data corresponding to at least one cell parameter and/or at least one function of the reference cells and/or the populations of reference cells, or (iii) both (i) and (ii).
912. The non-transitory computer-readable storage medium of any one of embodiments 893-911, wherein:
   (i) the input data comprises one or more quantitative assay readouts from the cell or the population of cells and the one or more quantitative assay readouts for the cell or the population of cells comprises one or more single values (e.g., one or more numerical values), one or more complex values (e.g., comprising a plurality of values) (e.g., comprising a range of values) [e.g., comprising multiple separate numerical values (e.g., pixel data from one or more images and/or flow plot data (e.g., from a gel))], one or more qualitative or semi-quantitative values [e.g., comprising one or more non-numerical values (e.g., a qualitative scale)], or a combination thereof (e.g., determined using a (e.g., computer-implemented) method in accordance with any one of embodiments 1-538),
   (ii) the reference data comprises one or more quantitative assay readouts from the reference cells and/or the populations of reference cells and the one or more quantitative assay readouts for the reference cells and/or the populations of reference cells comprises one or more single values (e.g., one or more numerical values), one or more complex values (e.g., comprising a plurality of values) (e.g., comprising a range of values) [e.g., comprising multiple separate numerical values (e.g., pixel data from one or more images and/or flow plot data (e.g., from a gel))], one or more qualitative or semi-quantitative values [e.g., comprising one or more non-numerical values (e.g., a qualitative scale)], or a combination thereof (e.g., determined using a (e.g., computer-implemented) method in accordance with any one of embodiments 1-538), or
   (iii) both (i) and (ii).
913. The non-transitory computer-readable storage medium of embodiment 912, wherein the one or more quantitative assay readouts comprises the complex values and/or the qualitative or semi-quantitative values and the complex values and/or the qualitative or semi-quantitative values comprises pixel information from one or more images (e.g., wherein the one or more quantitative assay readouts comprises one or more images from one or more assays), flow plot data (e.g., wherein the one or more quantitative assay readouts comprises one or more images from one or more assays), or a combination thereof (e.g., determined using a(e.g., computer-implemented) method in accordance with any one of embodiments 1-538).
914. The non-transitory computer-readable storage medium of embodiment 912 or embodiment 913, wherein the input data and/or the reference data comprises one or more single values that have been converted from one or more complex values and/or qualitative or semi-quantitative values of one or more quantitative assay readouts (e.g., from the cell or the population of cells and/or from the reference cells and/or the populations of reference cells) (e.g., prior to determining the predicted function) [e.g., using a predetermined conversion scheme (e.g., scale) (e.g., by combining (e.g., concatenating) and/or statistically processing (e.g., averaging), by the one or more processors, the values)].
915. The non-transitory computer-readable storage medium of embodiment 912 or embodiment 913, wherein the one or more quantitative assay readouts (e.g., from the cell or the population of cells and/or from the reference cells and/or the populations of reference cells) comprise the complex values and/or the qualitative or semi-quantitative values and the method comprises converting, by the one or more processors, the complex values and/or the qualitative or semi-quantitative values to a single value for each of the one or more quantitative assay readouts (e.g., prior to determining the predicted function) [e.g., using a predetermined conversion scheme (e.g., scale) (e.g., by combining (e.g., concatenating) and/or statistically processing (e.g., averaging), by the one or more processors, the values)].
916. The non-transitory computer-readable storage medium of any one of embodiments 893-915, wherein determining the predicted function comprises comparing, by the one or more processors, one or more single values for one or more quantitative assay readouts for the cell or the population of cells and one or more single values for one or more quantitative assay readouts for the reference cells and/or the populations of reference cells (e.g., after converting, by the one or more processors, to the single value(s) from one or more complex values and/or one or more qualitative and/or semi-quantitative values).
917. The non-transitory computer-readable storage medium of any one of embodiments 893-916, wherein the method comprises converting, by the one or more processors, one or more quantitative assay readouts into a suitable format for performing the determining (e.g., for input into a machine learning module) [e.g., suitable data format and/or size (e.g., string and/or number format and/or size)].
918. The non-transitory computer-readable storage medium of any one of embodiments 893-917, wherein determining the predicted function comprises using one or more weights to weight the input data and/or the reference data (e.g., relative to each other and/or to differently weight different portions of the input data and/or to differently weight different portions of the reference data) (e.g., used in a loss function used in a machine learning module) [e.g., that have been determined by a trained algorithm (e.g., in a machine learning module)].
919. The non-transitory computer-readable storage medium of embodiment 918, wherein the method comprises determining, by the one or more processors, the one or more weights.
920. The non-transitory computer-readable storage medium of embodiment 918 or embodiment 919, wherein the one or more weights are for weighting one or more quantitative assay readouts comprised in the input data and/or the reference data.
921. The non-transitory computer-readable storage medium of any one of embodiments 918-920, wherein determining the one or more weights comprises receiving, by the one or more processors, user input corresponding to the one or more weights (e.g., via one or more graphical user interfaces (GUIs)).
922. The non-transitory computer-readable storage medium of embodiment 921, wherein the user input comprises one or more of the one or more weights and/or one or more cell characteristics (e.g., function(s) and/or parameter(s)) to weight.
923. The non-transitory computer-readable storage medium of any one of embodiments 918-922, wherein determining the one or more weights comprises determining the one or more weights using a trained algorithm or training an algorithm (e.g., in a machine learning module).
924. The non-transitory computer-readable storage medium of any one of embodiments 918-923, wherein the one or more weights correspond to (e.g., wherein the user input represents):
   (i) one or more cell characteristics to consider or not consider (e.g., one or more quantitative assay readouts to consider or not consider),
   (ii) a preference of one or more cell characteristics over one or more other cell characteristics [e.g., a ranked list of two or more cell characteristics (e.g., a ranked list of some but not all or all available cell characteristics to choose from)](e.g., a preference of one or more quantitative assay readouts over one or more other assay readouts),
   (iii) a selection of one or more subsets of the input data and/or the reference data to weight more heavily or less heavily (e.g., comprising one or more desired weights [e.g., that is/are then fixed or that serves as an initial value that is/are then modified (e.g., through iteration in a machine learning module)], or
   (iv) a combination thereof.
925. The non-transitory computer-readable storage medium of any one of embodiments 893-924, wherein the method comprises comparing, by the one or more processors, the input data and (e.g., to) the reference data (e.g., using a machine learning module).
926. The non-transitory computer-readable storage medium of embodiment 925, wherein comparing the input data and the reference data comprises performing, by the one or more processors, a multiparametric comparison between the input data and the reference data.
927. The non-transitory computer-readable storage medium of embodiment 926, wherein the method comprises comparing (e.g., optimizing), by the one or more processors, two or more, three or more, four or more, five or more, six or more, seven or more, eight or more, nine or more, or ten or more parameters.
928. The non-transitory computer-readable storage medium of embodiment 926 or embodiment 927, wherein the multiparametric comparison is a biomarker.
929. The non-transitory computer-readable storage medium of any one of embodiments 893-928, wherein the predicted function comprises one or more of functions selected from the group consisting of durability of cell growth, durability of cell response, and cellular ability to elicit adaptive and innate immune responses.
930. The non-transitory computer-readable storage medium of any one of embodiments 893-929, wherein the method comprises determining (e.g., classifying and/or inferring), by the one or more processors. (e.g., wherein a machine learning module that determines the predicted function outputs) a categorization of the cell or the population of cells that is indicative of the predicted function of the cell or the population of cells.
931. The non-transitory computer-readable storage medium of any one of embodiments 893-930, wherein the predicted function comprises a categorization of the cell or the population of cells.
932. The non-transitory computer-readable storage medium of embodiment 930 or embodiment 931, wherein the categorization is a qualitative categorization (e.g., that uses a qualitative scale comprising one or more of: ‘poor,’ ‘usable,’ ‘good,’ and ‘exceptional’).
933. The non-transitory computer-readable storage medium of embodiment 930 or embodiment 931, wherein the categorization uses a quantitative scale (e.g., provides a numerical value on a predetermined scale).
934. The non-transitory computer-readable storage medium of any one of embodiments 930-933, wherein the categorization is indicative of a suitability of the cell or the population of cells for use as donor cell(s) in a cell therapy.
935. The non-transitory computer-readable storage medium of any one of embodiments 893-934, determining (e.g., classifying and/or inferring), by the one or more processors, suitability of the cell or the population of cells for use as donor cell(s) in a cell therapy (e.g., wherein the predicted function is indicative of the suitability) (e.g., wherein the suitability is determined using the predicted function) (e.g., using the input data and the reference data in a (e.g., computer-implemented) method to determine suitability in accordance with any one of embodiments 1-538).
936. The non-transitory computer-readable storage medium of embodiment 935, wherein determining the suitability comprises determining (e.g., classifying and/or inferring), by the one or more processors, that the cell or the population of cells is/are suitable for use as donor cell(s) (e.g., by a (e.g., computer-implemented) method in accordance with any one of embodiments 1-538).
937. The non-transitory computer-readable storage medium of embodiment 935 or embodiment 936, wherein the input data comprises data corresponding to one or more cell characteristics (e.g., function(s) and/or parameter(s)) and the method comprises determining (e.g., classifying and/or inferring), by the one or more processors that the cell or the population of cells is/are suitable for use as donor cell(s) due, in part, to presence and/or absence of one or more traits from the one or more cell characteristics (e.g., by a (e.g., computer-implemented) method in accordance with any one of embodiments 1-538).
938. The non-transitory computer-readable storage medium of embodiment 937, wherein the one or more cell characteristics correspond to age of subject from which the cell or the population of cells is derived, health history [e.g., health status (e.g., current health status)] of the subject, sex of the subject, or a combination thereof [e.g., and the one or more traits correspond to an acceptable and/or desirable (e.g., preferred) age or age range, acceptable and/or desirable (e.g., preferred) sex, presence of one or more acceptable and/or desirable (e.g., preferred) health traits, absence of one or more unacceptable and/or undesirable (e.g., non-preferred) health traits, or a combination thereof].
939. The non-transitory computer-readable storage medium of embodiment 937 or embodiment 938, wherein the method comprises determining (e.g., classifying and/or inferring), by the one or more processors, that the cell or the population of cells is suitable due to the presence and/or absence of the one or more traits when the cell or the population of cells would otherwise be less suitable or unsuitable without and/or with having the one or more traits, respectively (e.g., by a (e.g., computer-implemented) method in accordance with any one of embodiments 1-538).
940. The non-transitory computer-readable storage medium of any one of embodiments 935-939, wherein determining the suitability comprises using one or more weights to weight the input data and/or the reference data (e.g., relative to each other and/or to differently weight different portions of the input data and/or to differently weight different portions of the reference data) (e.g., used in a loss function used in a machine learning module).
941. The non-transitory computer-readable storage medium of embodiment 940, wherein the method comprises determining, by the one or more processors, one or more weights for use in determining the suitability (e.g., used in a loss function used in a machine learning module).
942. The non-transitory computer-readable storage medium of embodiment 940 or embodiment 941, wherein the one or more weights correspond to one or more desirable cell characteristics and/or the one or more weights have been selected (e.g., by user input) to more heavily weight one or more desirable cell characteristics than one or more other cell characteristics (e.g., wherein the one or more desirable cell characteristics are or have been determined, by the one or more processors, using reference data that comprises cell performance data).
943. The non-transitory computer-readable storage medium of any one of embodiments 940-942, wherein the one or more weights are for weighting one or more quantitative assay readouts comprised in the input data and/or the reference data.
944. The non-transitory computer-readable storage medium of any one of embodiments 940-943, wherein determining the one or more weights comprises receiving, by the one or more processors, user input corresponding to the one or more weights (e.g., via one or more graphical user interfaces (GUIs)).
945. The non-transitory computer-readable storage medium of embodiment 944, wherein the user input comprises one or more of the one or more weights and/or one or more cell characteristics to weight.
946. The non-transitory computer-readable storage medium of any one of embodiments 940-945, wherein the one or more weights correspond to (e.g., wherein the user input represents):
   (i) one or more cell characteristics to consider or not consider (e.g., one or more quantitative assay readouts to consider or not consider),
   (ii) a preference of one or more cell characteristics over one or more other cell characteristics [e.g., a ranked list of two or more cell characteristics (e.g., a ranked list of some but not all or all available cell characteristics to choose from)](e.g., a preference of one or more quantitative assay readouts over one or more other assay readouts),
   (iii) a selection of one or more subsets of the input data and/or the reference data to weight more heavily or less heavily (e.g., comprising one or more desired weights [e.g., that is/are then fixed or that serves as an initial value that is/are then modified (e.g., through iteration in a machine learning module)], or
   (iv) a combination thereof.
947. The non-transitory computer-readable storage medium of any one of embodiments 893-946, wherein the method is performed using (e.g., wherein the predicted function of the cell or the population of cells is determined using) a machine learning module (e.g., stored in the medium) that (a) has been trained using the reference data and (b) receives the input data as an input [e.g., in addition to one or more other inputs (e.g., user-selected weight(s) to be applied (e.g., in a loss function))].
948. The non-transitory computer-readable storage medium of embodiment 947, wherein the machine learning module employs a regression-based model (e.g., a logistic regression model), a regularization-based model (e.g., an elastic net model or a ridge regression model), an instance-based model (e.g., a support vector machine or a k-nearest neighbor model), a Bayesian-based model (e.g., a naive-based model or a Gaussian naive-based model), a clustering-based model (e.g., an expectation maximization model), an ensemble-based model (e.g., an adaptive boosting model, a random forest model, a bootstrap-aggregation model, or a gradient boosting machine model), or a neural-network-based model (e.g., a convolutional neural network, a recurrent neural network, autoencoder, a back propagation network, or a stochastic gradient descent network).
949. A cell therapy product comprising a population of cells, wherein
   i. the population includes cells with hypoimmune gene modifications (HIP cells) that exhibit reduced expression of one or more molecules of the MHC class I and/or MHC class II molecules and optionally also exhibit increased expression of at least one tolerogenic factor; and
   ii. at least 30%, at least 40%, at least 50%, at least 60%, at least 70%, at least 80%, or at least 90% of cells in the population exhibit reduced expression of one or more molecules of the MHC class I and/or MHC class II molecules and optionally also exhibit increased expression of at least one tolerogenic factor.
950. A pharmaceutical composition comprising a population of cells, wherein:
   i. the population includes cells with hypoimmune gene modifications (HIP cells) that exhibit reduced expression of one or more molecules of the MHC class I and/or MHC class II molecules and optionally also exhibit increased expression of at least one tolerogenic factor; and
   ii. at least 30%, at least 40%, at least 50%, at least 60%, at least 70%, at least 80%, or at least 90% of cells in the population exhibit reduced expression of one or more molecules of the MHC class I and/or MHC class II molecules and optionally also exhibit increased expression of at least one tolerogenic factor.
951. A cell therapy product according to embodiment 949 or a pharmaceutical composition according to embodiment 950, wherein at least 5%, at least 10%, at least 20%, at least 30%, at least 40%, at least 50%, at least 60%, at least 70% of cells in the population of cells do not exhibit reduced expression of one or more molecules of the MHC class I and/or MHC class II molecules and optionally also do not exhibit increased expression of at least one tolerogenic factor.
952. A cell therapy product according to embodiment 949 or 951 or a pharmaceutical composition according to embodiment 950 or 950, wherein 30-90%, 30-80%, 30-70%, 30-60%, 30-50% or 40-50% of cells in the population of cells are HIP cells that exhibit reduced expression of one or more molecules of the MHC class I and/or MHC class II molecules and optionally increased expression of at least one tolerogenic factor.
953. A cell therapy product according to any one of embodiments 949. 951 or 952 or a pharmaceutical composition according to any one of embodiments 950-952, wherein the HIP cells are as defined in any one of embodiments 212-368.
954. A cell therapy product according to any one of embodiments 949 or 951-953 or a pharmaceutical composition according to any one of embodiments 950-953, wherein the HIP cells further comprise a suicide gene or suicide switch as defined in any one of embodiments 382-393 or 720-724.
955. A cell therapy product according to any one of embodiments 949 or 951-954 or a pharmaceutical composition according to any one of embodiments 950-954, where the cells are beta cells as defined in any one of embodiments 484-492.
956. A cell therapy product according to any one of embodiments 949 or 951-954 or a pharmaceutical composition according to any one of embodiments 950-954, where the cells are hepatocytes as defined in any one of embodiments 504-507.
957. A cell therapy product according to any one of embodiments 949 or 951-954 or a pharmaceutical composition according to any one of embodiments 950-954, where the cells are neural cells as defined in any one of embodiments 508-512.
958. A cell therapy product according to any one of embodiments 949 or 951-954 or a pharmaceutical composition according to any one of embodiments 950-954, where the cells are endothelial cells or epithelial cells.
959. A cell therapy product according to any one of embodiments 949 or 951-954 or a pharmaceutical composition according to any one of embodiments 950-954, where the cells are cardiac cells (e.g., cardiomyocytes) as defined in any one of embodiments 513-517.
960. A cell therapy product according to any one of embodiments 949 or 951-954 or a pharmaceutical composition according to any one of embodiments 950-954, wherein 70-100%, 80-100%, or 90-100% of cells in the population of cells express a CAR.
961. A cell therapy product according to any one of embodiments 949 or 951-954 or 960 or a pharmaceutical composition according to any one of embodiments 950-954 or 960, where the cells are T cells as defined in any one of embodiments 465-477.
962. A cell therapy product according to any one of embodiments 949 or 951-954 or 960 or a pharmaceutical composition according to any one of embodiments 950-954 or 960, where the cells are NK cells as defined in any one of embodiments 478-483.
963. A cell therapy product according to any one of embodiments 949 or 951-954 or 960 or a pharmaceutical composition according to any one of embodiments 950-954 or 960, where the cells are macrophages as defined in any one of embodiments 500-503a.
964. A cell therapy product according to any one of embodiments 949 or 951-954 or 960 or a pharmaceutical composition according to any one of embodiments 950-954 or 960, where the cells are B cells as defined in any one of embodiments 493-499
965. A cell therapy product or a pharmaceutical composition according to embodiment any one of embodiments 949-964, for use in therapy.
966. A cell therapy product or a pharmaceutical composition according to embodiment any one of embodiments 949-964, for use in a method of treating a disease or condition.
967. A method of treating a disease or condition in a subject comprising administering to the subject a cell therapy product or a pharmaceutical composition according to embodiment any one of embodiments 949-964.
968. A cell therapy product or a pharmaceutical composition for use according to embodiment 965 or 966 or a method of treating a disease or condition according to embodiment 967, wherein the subject to be treated is not immunosuppressed i.e., has not been administered one or more immunosuppressive agents.
969. A cell therapy product or a pharmaceutical composition for use according to embodiment 965 or 966 or a method of treating a disease or condition according to embodiment 967, wherein the cell and the disease or condition are as defined in any one of embodiments 658-685.
970. A method of making a cell therapy product according to embodiment 599, the method comprising:
   a. isolating cells from a donor or population of donors; and
   b. manipulating the isolated cells to produce a cell therapy product.
971. The method of embodiment 970, further comprising cryopreserving the cell therapy product, and
   wherein cell viability of the cell therapy product after cryopreservation is at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.
972. The method of embodiment 971, wherein the cell viability of the cell therapy product after cryopreservation is at least about 80%.
973. The method of embodiment 971 or embodiment 972, wherein the cell viability of the cell therapy product after cryopreservation is at least about 90%.
974. The method of any of embodiments 971-973, wherein the cell viability of the cell therapy product after cryopreservation is at least about 95%.
975. The method of embodiment 970, wherein the manipulation comprises transducing the isolated cells with a viral vector, and
   wherein transduction efficiency of the isolated cells is at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.
976. The method of embodiment 975, wherein transduction efficiency of the isolated cells is at least about 40%.
977. The method of embodiment 975 or embodiment 976, wherein transduction efficiency of the isolated cells is at least about 45%.
978. The method of any of embodiments 975-977, wherein transduction efficiency of the isolated cells is at least about 50%.
979. The method of any of embodiments 975-978, wherein transduction efficiency of the isolated cells is at least about 55%.
980. The method of any of embodiments 975-979, wherein transduction efficiency of the isolated cells is at least about 60%.
981. The method of any of embodiments 975-980, wherein transduction efficiency of the isolated cells is at least about 65%.
982. The method of embodiment 970, wherein the manipulation comprises transducing the isolated cells with a viral vector, and
   wherein viral copy number (VCN) in the transduced cells is no more than about 5.0, no more than about 4.7, no more than about 4.5, no more than about 4.2, no more than about 4.0, no more than about 3.7, no more than about 3.5, no more than about 3.2, no more than about 3.0, no more than about 2.7, no more than about 2.5, no more than about 2.2, no more than about 2, no more than about 1.7, no more than about 1.5, no more than about 1.2, no more than about 1.0, no more than about 0.7, no more than about 0.5, no more than about 0.2, or no more than about 0.1.
983. The method of embodiment 982, wherein the VCN in the transduced cells is no more than about 3.0.
984. The method of embodiment 982 or embodiment 983, wherein the VCN in the transduced cells is no more than about 2.7.
985. The method of any of embodiments 982-984, wherein the VCN in the transduced cells is no more than about 2.5.
986. The method of any of embodiments 982-985, wherein the VCN in the transduced cells is no more than about 2.2.
987. The method of any of embodiments 982-986, wherein the VCN in the transduced cells is no more than about 2.0.
988. The method of embodiment 970, wherein the manipulation comprises modifying the genome of the isolated cells by inactivating or disrupting the TRAC gene locus and/or the TRBC gene locus and selecting the genome modified cells.
989. The method of embodiment 988, wherein the inactivation or disruption comprises inactivation or disruption of:
   a. one TRAC allele or both TRAC alleles, and/or
   b. one TRBC allele or both TRBC alleles.
990. The method of embodiment 988 or embodiment 989, wherein the percentage of residual TCRαβ+ cells in the selected cells is no more than about 1.0%, no more than about 0.9%, no more than about 0.8%, no more than about 0.7%, no more than about 0.6%, no more than about 0.5%, no more than about 0.4%, no more than about 0.3%, no more than about 0.2%, or no more than about 0.1%.
991. The method of any of embodiments 988-990, wherein the percentage of residual TCRαβ+ cells in the selected cells is no more than about 0.5%.
992. The method of any of embodiments 988-991, wherein the percentage of residual TCRαβ+ cells in the selected cells is no more than about 0.4%.
993. The method of any of embodiments 988-992, wherein the percentage of residual TCRαβ+ cells in the selected cells is no more than about 0.3%.
994. The method of any of embodiments 988-993, wherein the percentage of residual TCRαβ+ cells in the selected cells is no more than about 0.2%.
995. The method of any of embodiments 988-994, wherein the percentage of residual TCRαβ+ cells in the selected cells is no more than about 0.1%.
996. The method of embodiment 970, wherein the manipulation comprises preparing the cell therapy product with CD4+ cells and/or CD8+ cells.
997. The method of embodiment 996, wherein the final percentage of CD4+ cells in the cell therapy product is at least about 20%, at least about 25%, at least about 30%, at least about 35%, at least about 40%, at least about 45%, at least about 50%, at least about 55%, at least about 60%, at least about 65%, at least about 70%, or at least about 75%.
998. The method of embodiment 996 or embodiment 997, wherein the final percentage of CD8+ cells in the cell therapy product is at least about 20%, at least about 25%, at least about 30%, at least about 35%, at least about 40%, at least about 45%, at least about 50%, at least about 55%, at least about 60%, at least about 65%, at least about 70%, or at least about 75%.
999. The method of any one of embodiments 996-998, wherein the final percentage of CD4+ cells in the cell therapy product is at least about 30%.
1000. The method of any one of embodiments 996-999, wherein the final percentage of CD4+ cells in the cell therapy product is at least about 35%.
1001. The method of any one of embodiments 996-1000, wherein the final percentage of CD4+ cells in the cell therapy product is at least about 40%.
1002. the method of any one of embodiments 996-1001, wherein the final percentage of CD4+ cells in the cell therapy product is at least about 45%.
1003. The method of any one of embodiments 996-1002, wherein the final percentage of CD4+ cells in the cell therapy product is at least about 50%.
1004. The method of any one of embodiments 996-1003, wherein the final percentage of CD4+ cells in the cell therapy product is at least about 55%.
1005. The method of any one of embodiments 996-1004, wherein the final percentage of CD4+ cells in the cell therapy product is at least about 60%.
1006. The method of any one of embodiments 996-1005, wherein the final percentage of CD4+ cells in the cell therapy product is at least about 65%.
1007. The method of any one of embodiments 996-1006, wherein the final percentage of CD8+ cells in the cell therapy product is at least about 30%.
1008. The method of any one of embodiments 996-1007, wherein the final percentage of CD8+ cells in the cell therapy product is at least about 35%.
1009. The method of any one of embodiments 996-1008, wherein the final percentage of CD8+ cells in the cell therapy product is at least about 40%.
1010. The method of any one of embodiments 996-1009, wherein the final percentage of CD8+ cells in the cell therapy product is at least about 45%.
1011. The method of any one of embodiments 996-1010, wherein the final percentage of CD8+ cells in the cell therapy product is at least about 50%.
1012. The method of any one of embodiments 996-1011, wherein the final percentage of CD8 cells in the cell therapy product is at least about 55%.
1013. The method of any one of embodiments 996-1012, wherein the final percentage of CD8 cells in the cell therapy product is at least about 60%.
1014. The method of any one of embodiments 996-1013, wherein the final percentage of CD8 cells in the cell therapy product is at least about 65%.
1015. The method of any one of embodiments 996-1014, wherein the final percentage of CD8 cells in the cell therapy product is at least about 70%.
1016. The method of any one of embodiments 996-1015, wherein the final ratio of CD4+ cells to CD8+ cells in the cell therapy product is about 0.4:1, about 0.45:1, about 0.5:1, about 0.55:1, about 0.6:1, about 0.65:1, about 0.7:1, about 0.75:1, about 0.8:1, about 0.85:1, about 0.9:1, about 0.95:1, about 1:1, about 1:0.95, about 1:0.9, about 1:0.85, about 1:0.8, about 1:0.75, about 1:0.7, about 1:0.65, about 1:0.6, about 1:0.55, about 1:0.5, about 1:0.45, or about 1:0.4.
1017. The method of any one of embodiments 996-1016, wherein the final ratio of CD4+ cells to CD8+ cells in the cell therapy product is about 0.45:1.
1018. The method of any one of embodiments 996-1017, wherein the final ratio of CD4+ cells to CD8+ cells in the cell therapy product is about 0.50:1.
1019. The method of any one of embodiments 996-1018, wherein the final ratio of CD4+ cells to CD8+ cells in the cell therapy product is about 0.55:1.
1020. The method of any one of embodiments 996-1019, wherein the final ratio of CD4+ cells to CD8+ cells in the cell therapy product is about 0.60:1.
1021. The method of any one of embodiments 996-1020, wherein the final ratio of CD4+ cells to CD8+ cells in the cell therapy product is about 0.65:1.
1022. The method of any one of embodiments 996-1021, wherein the final ratio of CD4+ cells to CD8+ cells in the cell therapy product is about 1:0.60.
1023. The method of any one of embodiments 996-1022, wherein the final ratio of CD4+ cells to CD8+ cells in the cell therapy product is about 1:0.65.
1024. The method of any one of embodiments 996-1023, wherein the final ratio of CD4+ cells to CD8+ cells in the cell therapy product is about 1:0.70.
1025. The method of any one of embodiments 996-1024, wherein the final ratio of CD4+ cells to CD8+ cells in the cell therapy product is about 1:0.75.
1026. The method of any one of embodiments 996-1025, wherein the final ratio of CD4+ cells to CD8+ cells in the cell therapy product is about 1:0.80.
1027. The method of any one of embodiments 996-1026, wherein the final ratio of CD4+ cells to CD8+ cells in the cell therapy product is about 1:0.85.
1028. The method of any one of embodiments 996-1027, wherein the final ratio of CD4+ cells to CD8+ cells in the cell therapy product is about 1:0.90.
1029. The method of embodiment 970, wherein the manipulation comprises preparing the cell therapy product with T_{CM} cells (CD45RO+CCR7+CD95+) and/or T_{EM} cells (CD45RO+CCR7-CD95+).
1030. The method of embodiment 1029, wherein the final percentage of T_{CM} cells in the cell therapy product is at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%.
1031. The method of embodiment 1029 or embodiment 1030, wherein the final percentage of T_{EM} cells in the cell therapy product is at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%.
1032. The method of any of embodiments 1029-1031, wherein the final percentage of T_{CM} cells in the cell therapy product is at least about 30%.
1033. The method of any of embodiments 1029-1032, wherein the final percentage of T_{CM} cells in the cell therapy product is at least about 35%.
1034. The method of any of embodiments 1029-1033, wherein the final percentage of T_{CM} cells in the cell therapy product is at least about 40%.
1035. The method of any of embodiments 1029-1034, wherein the final percentage of T_{CM} cells in the cell therapy product is at least about 45%.
1036. The method of any of embodiments 1029-1035, wherein the final percentage of T_{CM} cells in the cell therapy product is at least about 50%.
1037. The method of any of embodiments 1029-1036, wherein the final percentage of T_{EM} cells in the cell therapy product is at least about 50%.
1038. The method of any of embodiments 1029-1037, wherein the final percentage of T_{EM} cells in the cell therapy product is at least about 55%.
1039. The method of any of embodiments 1029-1038, wherein the final percentage of T_{EM} cells in the cell therapy product is at least about 60%.
1040. The method of any of embodiments 1029-1039, wherein the final percentage of T_{EM} cells in the cell therapy product is at least about 65%.
1041. The method of any of embodiments 1029-1039, wherein the final percentage of T_{EM} cells in the cell therapy product is at least about 70%.
1042. The method of embodiment 970, wherein the manipulation comprises modifying the genome of the isolated cells by inactivating or disrupting the TRAC gene locus.
1043. The method of embodiment 1042, wherein the inactivation or disruption comprises inactivation or disruption of one TRAC allele or both TRAC alleles.
1044. The method of embodiment 1042 or embodiment 1043, wherein the TRAC inactivation or disruption efficiency is at least about 60%, at least about 65%, at least about 70%, at least about 75%, at least about 80%, at least about 85%, at least about 90%, at least about 95%, at least about 96%, at least about 97%, at least about 98%, or at least about 99%.
1045. The method of any of embodiments 1042-1044, wherein the TRAC inactivation or disruption efficiency is at least about 95%.
1046. The method of any of embodiments 1042-1045, wherein the TRAC inactivation or disruption efficiency is at least about 96%.
1047. The method of any of embodiments 1042-1046, wherein the TRAC inactivation or disruption efficiency is at least about 97%.
1048. The method of any of embodiments 1042-1047, wherein the TRAC inactivation or disruption efficiency is at least about 98%.
1049. The method of any of embodiments 1042-1048, wherein the TRAC inactivation or disruption efficiency is at least about 99%.
1050. The method of embodiment 970, wherein the manipulation comprises modifying the genome of the isolated cells by inactivating or disrupting the B2M gene locus.
1051. The method of embodiment 1050, wherein the inactivation or disruption comprises inactivation or disruption of one B2M allele or both B2M alleles.
1052. The method of embodiment 1050 or embodiment 1001, wherein the B2M inactivation or disruption efficiency is at least about 60%, at least about 65%, at least about 70%, at least about 75%, at least about 80%, at least about 85%, at least about 90%, at least about 95%, at least about 96%, at least about 97%, at least about 98%, or at least about 99%.
1053. The method of any of embodiments 1050-1052, wherein the B2M inactivation or disruption efficiency is at least about 87%.
1054. The method of any of embodiments 1050-1053, wherein the B2M inactivation or disruption efficiency is at least about 88%.
1055. The method of any of embodiments 1050-1054, wherein the B2M inactivation or disruption efficiency is at least about 89%.
1056. The method of any of embodiments 1050-1055, wherein the B2M inactivation or disruption efficiency is at least about 90%.
1057. The method of any of embodiments 1050-1056, wherein the B2M inactivation or disruption efficiency is at least about 91%.
1058. The method of embodiment 970, wherein the manipulation comprises modifying the genome of the isolated cells by inactivating or disrupting the CIITA gene locus.
1059. The method of embodiment 1058, wherein the inactivation or disruption comprises inactivation or disruption of one CIITA allele or both CIITA alleles.
1060. The method of embodiment 1058 or embodiment 1059, wherein the CIITA inactivation or disruption efficiency is at least about 60%, at least about 65%, at least about 70%, at least about 75%, at least about 80%, at least about 85%, at least about 90%, at least about 95%, at least about 96%, at least about 97%, at least about 98%, or at least about 99%.
1061. The method of any one of embodiments 1058-1060, wherein the CIITA inactivation or disruption efficiency is at least about 90%.
1062. The method of any one of embodiments 1058-1061, wherein the CIITA inactivation or disruption efficiency is at least about 91%.
1063. The method of any one of embodiments 1058-1062, wherein the CIITA inactivation or disruption efficiency is at least about 92%.
1064. The method of any one of embodiments 1058-1063, wherein the CIITA inactivation or disruption efficiency is at least about 93%.
1065. The method of any one of embodiments 1058-1064, wherein the CIITA inactivation or disruption efficiency is at least about 94%.
1066. The method of any one of embodiments 1058-1065, wherein the CIITA inactivation or disruption efficiency is at least about 95%.
1067. The method of any one of embodiments 1058-1066, wherein the CIITA inactivation or disruption efficiency is at least about 96%.
1068. The method of any one of embodiments 1058-1067, wherein the CIITA inactivation or disruption efficiency is at least about 97%.
1069. The method of any one of embodiments 988-1068, wherein inactivation or disruption of a gene locus is measured by cell surface expression.
1070. The method of any one of embodiments 970-1069, further comprising selecting the donor or population of donors according to a method defined in embodiments 1-388.
1071. The method of any one of embodiments 970-1070, wherein the isolated cells are selected from the group consisting of islet cells, beta islet cells, pancreatic islet cells, immune cells, B cells, T cells, natural killer (NK) cells, natural killer T (NKT) cells, macrophages, endothelial cells, muscle cells, cardiac muscle cells, smooth muscle cells, skeletal muscle cells, dopaminergic neurons, retinal pigmented epithelium cells (e.g., retinal pigmented epithelium (RPE) cells and thyroid cells), optic cells, hepatocytes, thyroid cells, skin cells, glial progenitor cells, neural cells (e.g., cerebral endothelial cells, dopaminergic neurons, glial cells, and hematopoietic stem cells (HSCS) cells), cardiac cells, stem cells, hematopoietic stem cells, induced pluripotent stem cells (iPSCs), mesenchymal stem cells (MSCs), embryonic stem cells (ESCs), pluripotent stem cells (PSCs), or blood cells.
1072. The method of any one of embodiments 970-1071, wherein the isolated cells are T cells.
1073. The method of embodiment 1072, wherein the T cells comprises a CAR.
1074. The method of embodiment 1073, wherein the T cells comprise CAR-T cells according to embodiment 349.
1075. A cell or a population of cells of embodiment 1074.
1076. A composition containing the cell or the population of cells of embodiment 1075.
1077. A method of making a CAR-T cell therapy product comprising providing a T cell or a population of T cells that have been evaluated, profiled, identified or selected according to a method defined in embodiments 1-538 and manufacturing a CAR-T cell therapy product therefrom.
1078. A method of making a CAR-T cell therapy product according to embodiment 1077, the method comprising:
   a. isolating cells from a donor or population of donors; and
   b. manipulating the isolated cells to produce a cell therapy product.
1079. The method of embodiment 1078, further comprising cryopreserving the cell therapy product, and wherein cell viability of the CAR-T cell therapy product after cryopreservation is at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.
1080. The method of embodiment 1079, wherein the cell viability of the CAR-T cell therapy product after cryopreservation is at least about 80%.
1081. The method of embodiment 1079 or embodiment 1080, wherein the cell of the CAR-T cell therapy product after cryopreservation is at least about 90%.
1082. The method of any of embodiments 1079-1081, wherein the cell viability of the CAR-T cell therapy product after cryopreservation is at least about 95%,
1083. The method of embodiment 1078, wherein the manipulation comprises transducing the isolated cells with a viral vector, and
   wherein transduction efficiency of the isolated cells is at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.
1084. The method of embodiment 1083, wherein transduction efficiency of the isolated cells is at least about 40%.
1085. The method of embodiment 1083 or embodiment 184, wherein transduction efficiency of the isolated cells is at least about 45%.
1086. The method of any of embodiments 1083-1085, wherein transduction efficiency of the isolated cells is at least about 50%.
1087. The method of any of embodiments 1083-1086, wherein transduction efficiency of the isolated cells is at least about 55%.
1088. The method of any of embodiments 1083-1087, wherein transduction efficiency of the isolated cells is at least about 60%.
1089. The method of any of embodiments 1083-1088, wherein transduction efficiency of the isolated cells is at least about 65%.
1090. The method of embodiment 1078, wherein the manipulation comprises transducing the isolated cells with a viral vector, and
   wherein viral copy number (VCN) in the transduced cells is no more than about 5.0, no more than about 4.7, no more than about 4.5, no more than about 4.2, no more than about 4.0, no more than about 3.7, no more than about 3.5, no more than about 3.2, no more than about 3.0, no more than about 2.7, no more than about 2.5, no more than about 2.2, no more than about 2, no more than about 1.7, no more than about 1.5, no more than about 1.2, no more than about 1.0, no more than about 0.7, no more than about 0.5, no more than about 0.2, or no more than about 0.1.
1091. The method of embodiment 1090, wherein the VCN in the transduced cells is no more than about 3.0.
1092. The method of embodiment 1090 or embodiment 1091, wherein the VCN in the transduced cells is no more than about 2.7.
1093. The method of any of embodiments 1090-1092, wherein the VCN in the transduced cells is no more than about 2.5.
1094. The method of any of embodiments 1090-1093, wherein the VCN in the transduced cells is no more than about 2.2.
1095. The method of any of embodiments 1090-1094, wherein the VCN in the transduced cells is no more than about 2.0.
1096. The method of embodiment 1078, wherein the manipulation comprises modifying the genome of the isolated cells by inactivating or disrupting the TRAC gene locus and/or the TRBC gene locus and selecting the genome modified cells.
1097. The method of embodiment 1096, wherein the inactivation or disruption comprises inactivation or disruption of:
   a. one TRAC allele or both TRAC alleles, and/or
   b. one TRBC allele or both TRBC alleles.
1098. The method of embodiment 1096 or embodiment 1097, wherein the percentage of residual TCRαβ+ cells in the selected cells is no more than about 1.0%, no more than about 0.9%, no more than about 0.8%, no more than about 0.7%, no more than about 0.6%, no more than about 0.5%, no more than about 0.4%, no more than about 0.3%, no more than about 0.2%, or no more than about 0.1%.
1099. The method of any of embodiments 1096-1098, wherein the percentage of residual TCRαβ+ cells in the selected cells is no more than about 0.5%.
1100. The method of any of embodiments 1096-1099, wherein the percentage of residual TCRαβ+ cells in the selected cells is no more than about 0.4%.
1101. The method of any of embodiments 1096-1100, wherein the percentage of residual TCRαβ+ cells in the selected cells is no more than about 0.3%.
1102. The method of any of embodiments 1096-1101, wherein the percentage of residual TCRαβ+ cells in the selected cells is no more than about 0.2%.
1103. The method of any of embodiments 1096-1102, wherein the percentage of residual TCRαβ+ cells in the selected cells is no more than about 0.1%.
1104. The method of embodiment 1078, wherein the manipulation comprises preparing the CAR-T cell therapy product with CD4+ cells and/or CD8+ cells.
1105. The method of embodiment 1104, wherein the final percentage of CD4+ cells in the CAR-T cell therapy product is at least about 20%, at least about 25%, at least about 30%, at least about 35%, at least about 40%, at least about 45%, at least about 50%, at least about 55%, at least about 60%, at least about 65%, at least about 70%, or at least about 75%.
1106. The method of embodiment 1104 or embodiment 1105, wherein the final percentage of CD8+ cells in the CAR-T cell therapy product is at least about 20%, at least about 25%, at least about 30%, at least about 35%, at least about 40%, at least about 45%, at least about 50%, at least about 55%, at least about 60%, at least about 65%, at least about 70%, or at least about 75%.
1107. The method of any one of embodiments 1104-1106, wherein the final percentage of CD4+ cells in the CAR-T cell therapy product is at least about 30%.
1108. The method of any one of embodiments 1104-1107, wherein the final percentage of CD4+ cells in the CAR-T cell therapy product is at least about 35%.
1109. The method of any one of embodiments 1104-1108, wherein the final percentage of CD4+ cells in the CAR-T cell therapy product is at least about 40%.
1110. The method of any one of embodiments 1104-1109, wherein the final percentage of CD4+ cells in the CAR-T cell therapy product is at least about 45%.
1111. The method of any one of embodiments 1104-1110, wherein the final percentage of CD4+ cells in the CAR-T cell therapy product is at least about 50%.
1112. The method of any one of embodiments 1104-1111, wherein the final percentage of CD4+ cells in the CAR-T cell therapy product is at least about 55%.
1113. The method of any one of embodiments 1104-1112, wherein the final percentage of CD4+ cells in the CAR-T cell therapy product is at least about 60%.
1114. The method of any one of embodiments 1104-1113, wherein the final percentage of CD4+ cells in the CAR-T cell therapy product is at least about 65%.
1115. The method of any one of embodiments 1104-1114, wherein the final percentage of CD8+ cells in the CAR-T cell therapy product is at least about 30%.
1116. The method of any one of embodiments 1104-1115, wherein the final percentage of CD8+ cells in the CAR-T cell therapy product is at least about 35%.
1117. The method of any one of embodiments 1104-1116, wherein the final percentage of CD8+ cells in the CAR-T cell therapy product is at least about 40%.
1118. The method of any one of embodiments 1104-1117, wherein the final percentage of CD8+ cells in the CAR-T cell therapy product is at least about 45%.
1119. The method of any one of embodiments 1104-1118, wherein the final percentage of CD8+ cells in the CAR-T cell therapy product is at least about 50%.
1120. The method of any one of embodiments 1104-1119, wherein the final percentage of CD8 cells in the CAR-T cell therapy product is at least about 55%.
1121. The method of any one of embodiments 1104-1120, wherein the final percentage of CD8 cells in the CAR-T cell therapy product is at least about 60%.
1122. The method of any one of embodiments 1104-1121, wherein the final percentage of CD8 cells in the CAR-T cell therapy product is at least about 65%.
1123. The method of any one of embodiments 1104-1122, wherein the final percentage of CD8 cells in the CAR-T cell therapy product is at least about 70%.
1124. The method of any one of embodiments 1104-1123, wherein the final ratio of CD4+ cells to CD8+ cells in the CAR-T cell therapy product is about 0.4:1, about 0.45:1, about 0.5:1, about 0.55:1, about 0.6:1, about 0.65:1, about 0.7:1, about 0.75:1, about 0.8:1, about 0.85:1, about 0.9:1, about 0.95:1, about 1:1, about 1:0.95, about 1:0.9, about 1:0.85, about 1:0.8, about 1:0.75, about 1:0.7, about 1:0.65, about 1:0.6, about 1:0.55, about 1:0.5, about 1:0.45, or about 1:0.4.
1125. The method of any one of embodiments 1104-1124, wherein the final ratio of CD4+ cells to CD8+ cells in the CAR-T cell therapy product is about 0.45:1.
1126. The method of any one of embodiments 1104-1125, wherein the final ratio of CD4+ cells to CD8+ cells in the CAR-T cell therapy product is about 0.50:1.
1127. The method of any one of embodiments 1104-1126, wherein the final ratio of CD4+ cells to CD8+ cells in the CAR-T cell therapy product is about 0.55:1.
1128. The method of any one of embodiments 1104-1127, wherein the final ratio of CD4+ cells to CD8+ cells in the CAR-T cell therapy product is about 0.60:1.
1129. The method of any one of embodiments 1104-1128, wherein the final ratio of CD4+ cells to CD8+ cells in the CAR-T cell therapy product is about 0.65:1.
1130. The method of any one of embodiments 1104-1129, wherein the final ratio of CD4+ cells to CD8+ cells in the CAR-T cell therapy product is about 1:0.60.
1131. The method of any one of embodiments 1104-1130, wherein the final ratio of CD4+ cells to CD8+ cells in the CAR-T cell therapy product is about 1:0.65.
1132. The method of any one of embodiments 1104-1131, wherein the final ratio of CD4+ cells to CD8+ cells in the CAR-T cell therapy product is about 1:0.70.
1133. The method of any one of embodiments 1104-1132, wherein the final ratio of CD4+ cells to CD8+ cells in the CAR-T cell therapy product is about 1:0.75.
1134. The method of any one of embodiments 1104-1133, wherein the final ratio of CD4+ cells to CD8+ cells in the CAR-T cell therapy product is about 1:0.80.
1135. The method of any one of embodiments 1104-1134, wherein the final ratio of CD4+ cells to CD8+ cells in the CAR-T cell therapy product is about 1:0.85.
1136. The method of any one of embodiments 1104-1135, wherein the final ratio of CD4+ cells to CD8+ cells in the CAR-T cell therapy product is about 1:0.90.
1137. The method of embodiment 1078, wherein the manipulation comprises preparing the CAR-T cell therapy product with T_{CM} cells (CD45RO+CCR7+CD95+) and/or T_{EM} cells (CD45RO+CCR7−CD95+).
1138. The method of embodiment 1137, wherein the final percentage of T_{CM} cells in the CAR-T cell therapy product is at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%.
1139. The method of embodiment 1137 or embodiment 1138, wherein the final percentage of T_{EM} cells in the CAR-T cell therapy product is at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%.
1140. The method of any of embodiments 1137-1139, wherein the final percentage of T_{CM} cells in the CAR-T cell therapy product is at least about 30%.
1141. The method of any of embodiments 1137-1140, wherein the final percentage of T_{CM} cells in the CAR-T cell therapy product is at least about 35%.
1142. The method of any of embodiments 1137-1141, wherein the final percentage of T_{CM} cells in the CAR-T cell therapy product is at least about 40%.
1143. The method of any of embodiments 1137-1142, wherein the final percentage of T_{CM} cells in the CAR-T cell therapy product is at least about 45%.
1144. The method of any of embodiments 1137-1143, wherein the final percentage of T_{CM} cells in the CAR-T cell therapy product is at least about 50%.
1145. The method of any of embodiments 1137-1144, wherein the final percentage of T_{EM} cells in the CAR-T cell therapy product is at least about 50%.
1146. The method of any of embodiments 1137-1145, wherein the final percentage of T_{EM} cells in the CAR-T cell therapy product is at least about 55%.
1147. The method of any of embodiments 1137-1146, wherein the final percentage of T_{EM} cells in the CAR-T cell therapy product is at least about 60%.
1148. The method of any of embodiments 1137-1147, wherein the final percentage of T_{EM} cells in the CAR-T cell therapy product is at least about 65%.
1149. The method of any of embodiments 1137-1148, wherein the final percentage of T_{EM} cells in the CAR-T cell therapy product is at least about 70%.
1150. The method of embodiment 1078, wherein the manipulation comprises modifying the genome of the isolated cells by inactivating or disrupting the TRAC gene locus.
1151. The method of embodiment 1150, wherein the inactivation or disruption comprises inactivation or disruption of one TRAC allele or both TRAC alleles.
1152. The method of embodiment 1150or embodiment 1151, wherein the TRAC inactivation or disruption efficiency is at least about 60%, at least about 65%, at least about 70%, at least about 75%, at least about 80%, at least about 85%, at least about 90%, at least about 95%, at least about 96%, at least about 97%, at least about 98%, or at least about 99%.
1153. The method of any of embodiments 1150-1152, wherein the TRAC inactivation or disruption efficiency is at least about 95%.
1154. The method of any of embodiments 1150-1153, wherein the TRAC inactivation or disruption efficiency is at least about 96%.
1155. The method of any of embodiments 1150-1154, wherein the TRAC inactivation or disruption efficiency is at least about 97%.
1156. The method of any of embodiments 1150-1155, wherein the TRAC inactivation or disruption efficiency is at least about 98%.
1157. The method of any of embodiments 1150-1156, wherein the TRAC inactivation or disruption efficiency is at least about 99%.
1158. The method of embodiment 1078, wherein the manipulation comprises modifying the genome of the isolated cells by inactivating or disrupting the B2M gene locus.
1159. The method of embodiment 1158, wherein the inactivation or disruption comprises inactivation or disruption of one B2M allele or both B2M alleles.
1160. The method of embodiment 1158 or embodiment 1159, wherein the B2M inactivation or disruption efficiency is at least about 60%, at least about 65%, at least about 70%, at least about 75%, at least about 80%, at least about 85%, at least about 90%, at least about 95%, at least about 96%, at least about 97%, at least about 98%, or at least about 99%.
1161. The method of any of embodiments 1158-1160, wherein the B2M inactivation or disruption efficiency is at least about 87%.
1162. The method of any of embodiments 1158-1161, wherein the B2M inactivation or disruption efficiency is at least about 88%.
1163. The method of any of embodiments 1158-1162, wherein the B2M inactivation or disruption efficiency is at least about 89%.
1164. The method of any of embodiments 1158-1163, wherein the B2M inactivation or disruption efficiency is at least about 90%.
1165. The method of any of embodiments 1158-1164, wherein the B2M inactivation or disruption efficiency is at least about 91%.
1166. The method of embodiment 1078, wherein the manipulation comprises modifying the genome of the isolated cells by inactivating or disrupting the CIITA gene locus.
1167. The method of embodiment 1166, wherein the inactivation or disruption comprises inactivation or disruption of one CIITA allele or both CIITA alleles.
1168. The method of embodiment 1166 or embodiment 1167, wherein the CIITA inactivation or disruption efficiency is at least about 60%, at least about 65%, at least about 70%, at least about 75%, at least about 80%, at least about 85%, at least about 90%, at least about 95%, at least about 96%, at least about 97%, at least about 98%, or at least about 99%.
1169. The method of any one of embodiments 1166-1168, wherein the CIITA inactivation or disruption efficiency is at least about 90%.
1170. The method of any one of embodiments 1166-1169, wherein the CIITA inactivation or disruption efficiency is at least about 91%.
1171. The method of any one of embodiments 1166-1170, wherein the CIITA inactivation or disruption efficiency is at least about 92%.
1172. The method of any one of embodiments 1166-1171, wherein the CIITA inactivation or disruption efficiency is at least about 93%.
1173. The method of any one of embodiments 1166-1172, wherein the CIITA inactivation or disruption efficiency is at least about 94%.
1174. The method of any one of embodiments 1166-1173, wherein the CIITA inactivation or disruption efficiency is at least about 95%.
1175. The method of any one of embodiments 1166-1174, wherein the CIITA inactivation or disruption efficiency is at least about 96%.
1176. The method of any one of embodiments 1166-1175, wherein the CIITA inactivation or disruption efficiency is at least about 97%.
1177. The method of any one of embodiments 1096-1176, wherein inactivation or disruption of a gene locus is measured by cell surface expression.
1178. The method of any one of embodiments 1078-1187, further comprising selecting the donor or population of donors according to a method defined in embodiments 1-538.
1179. The method of any one of embodiments 1078-1178, wherein the isolated cells are selected from the group consisting of islet cells, beta islet cells, pancreatic islet cells, immune cells, B cells, T cells, natural killer (NK) cells, natural killer T (NKT) cells, macrophages, endothelial cells, muscle cells, cardiac muscle cells, smooth muscle cells, skeletal muscle cells, dopaminergic neurons, retinal pigmented epithelium cells (e.g., retinal pigmented epithelium (RPE) cells and thyroid cells), optic cells, hepatocytes, thyroid cells, skin cells, glial progenitor cells, neural cells (e.g., cerebral endothelial cells, dopaminergic neurons, glial cells, and hematopoietic stem cells (HSCS) cells), cardiac cells, stem cells, hematopoietic stem cells, induced pluripotent stem cells (iPSCs), mesenchymal stem cells (MSCs), embryonic stem cells (ESCs), pluripotent stem cells (PSCs), or blood cells.
1180. The method of any one of embodiments 1078-1179, wherein the isolated cells are T cells.
1181. The method of embodiment 1180, wherein the T cells comprises a CAR.
1182. The method of embodiment 1181, wherein the T cells comprise CAR-T cells according to any one of embodiments 349-359 or 432-436.
1183. A cell or a population of cells of embodiment 1182.
1184. A composition containing the cell or the population of cells of embodiment 1183.

1. A method of making a hypoimmunogenic MAIT cell comprising introducing into a MAIT cell:
(i) one or more genetic alterations that reduce cell surface expression of a functional Major Histocompatibility Antigen Class I (HLA-I) complex as compared to a reference cell, and/or one or more genetic alterations that reduce cell surface expression of a functional Major Histocompatibility Antigen Class II (HLA-II) complex as compared to a reference cell, and
(ii) one or more genetic alterations that increase cell surface expression of CD47 as compared to a reference cell.

2. The method according to claim 1, wherein the method comprises introducing one or more genetic alterations into a MAIT cell that reduce expression of:
(i) B2M,
(ii) CIITA,
(iii) TCR (e.g., TRAC and/or TRB), or
(iv) any combination thereof,
as compared to a reference cell.

3. The method according to claim 1 or 2, wherein the method comprises introducing one or more genetic alterations into a MAIT cell that reduce cell surface expression of a functional HLA-I complex,
wherein the one or more genetic alterations that reduce cell surface expression of a functional HLA-I complex comprise the one or more genetic alterations that reduce expression of B2M, HLA-A, HLA-B, HLA-C, or any combination thereof, as compared to a reference cell.

4. The method according to any one of the preceding claims, wherein the method comprises introducing one or more genetic alterations into a MAIT cell that reduce cell surface expression of a functional HLA-II complex,
wherein the one or more genetic alterations that reduce cell surface expression of a functional HLA-II complex comprise the one or more genetic alterations that reduce expression of CIITA, HLA-DP, HLA-DQ, HLA-DR, HLA-DM, HLA-DO, or any combination thereof, as compared to a reference cell.

5. The method according to any one of the preceding claims, wherein the method comprises introducing one or more genetic alterations into a MAIT cell that reduce expression of TRB, optionally wherein TRB comprises TRBC1, TRBC2, or TRBC1 and TRBC2.

6. The method according to any one of the preceding claims, wherein the method comprises introducing one or more genetic alterations into a MAIT cell that reduce expression of one or more Y chromosome proteins, as compared to a reference cell, optionally wherein the one or more Y chromosome proteins comprise Protocadherin-11 Y-linked (PCDH11 Y) and/or Neuroligin-4 Y-linked (NLGN4Y).

7. The method according to any one of the preceding claims, wherein the method comprises introducing one or more genetic alterations into a MAIT cell that reduce expression of one or more additional molecules comprise one or more blood antigen proteins, as compared to a reference cell,
optionally wherein the one or more blood antigen proteins comprise ABO, FUT1 and/or RHD.

8. The method according to any one of the preceding claims, wherein the method comprises introducing one or more transgenes into the MAIT cell.

9. The method according to claim 8, wherein the one or more transgenes are part of one or more vectors.

10. The method according to claim 9, wherein at least one of the one or more vectors is a multicistronic vector.

11. The method according to any one of the preceding claims, wherein the method comprises introducing a chimeric antigen receptor (CAR) into a MAIT cell, optionally wherein the one or more transgenes comprise a transgene that encodes the CAR.

12. The method according to claim 11, wherein the CAR comprises a signal peptide, an extracellular binding domain specific to CD19, a hinge domain, a transmembrane domain, an intracellular costimulatory domain, and/or an intracellular signaling domain.

13. The method according to claim 11 or 12, wherein the CAR comprises a CD5-specific CAR, a CD19-specific CAR, a CD20-specific CAR, a CD22-specific CAR, a CD23-specific CAR, a CD30-specific CAR, a CD33-specific CAR, CD38-specific CAR, a CD70-specific CAR, a CD123-specific CAR, a CD138-specific CAR, a Kappa, Lambda, B cell maturation agent (BCMA)-specific CAR, a G-protein coupled receptor family C group 5 member D (GPRC5D)-specific CAR, a CD123-specific CAR, a LeY-specific CAR, a NKG2D ligand-specific CAR, a WT1-specific CAR, a GD2-specific CAR, a HER2-specific CAR, a EGFR-specific CAR, a EGFRvIII-specific CAR, a B7H3-specific CAR, a PSMA-specific CAR, a PSCA-specific CAR, a CAIX-specific CAR, a CD171-specific CAR, a CEA-specific CAR, a CSPG4-specific CAR, a EPHA2-specific CAR, a FAP-specific CAR, a FRα-specific CAR, a IL-13Rα-specific CAR, a Mesothelin-specific CAR, a MUC1-specific CAR, a MUC16-specific CAR, a ROR1-specific CAR, a C-Met-specific CAR, a CD133-specific CAR, a Ep-CAM-specific CAR, a GPC3-specific CAR, a HPV16-E6-specific CAR, a IL13Ra2-specific CAR, a MAGEA3-specific CAR, a MAGEA4-specific CAR, a MART1-specific CAR, a NY-ESO-1-specific CAR, a VEGFR2-specific CAR, a α-Folate receptor-specific CAR, a CD24-specific CAR, a CD44v7/8-specific CAR, a EGP-2-specific CAR, a EGP-40-specific CAR, a erb-B2-specific CAR, a erb-B 2,3,4-specific CAR, a FBP-specific CAR, a Fetal acethylcholine e receptor-specific CAR, a GD2-specific CAR, a GD3-specific CAR, a HMW-MAA-specific CAR, a IL-11Rα-specific CAR, a KDR-specific CAR, a Lewis Y-specific CAR, a L1-cell adhesion molecule-specific CAR, a MAGE-A1-specific CAR, a Oncofetal antigen (h5T4)-specific CAR, a TAG-72-specific CAR, or a CD19/CD22-bispecific CAR.

14. The method according to any one of claims 11-13, wherein the CAR comprises a CD19-specific CAR, a CD20-specific CAR, a CD22-specific CAR, a CD38-specific CAR, a CD123-specific CAR, a CD138-specific CAR, a BCMA-specific CAR, or a CD19/CD22-bispecific CAR.

15. The method according to claim 14, wherein the CAR is specific for CD19.

16. The method according to claim 14, wherein the CAR is specific for CD22.

17. The method according to 14, wherein the CAR is a CD19/CD22-bispecific CAR.

18. The method according to any one of the preceding claims, wherein the method comprises introducing one or more safety switches into a MAIT cell.

19. The method according to claim 18, wherein the one or more safety switches are capable of controlled killing of the cell or the population of cells.

20. The method according to any one of claims 59-67, wherein introducing one or more transgenes, a CAR, and/or one or more safety switches comprises integrating at least one of the one or more transgenes, a CAR, and/or one or more safety switches into the genome of the MAIT cell.

21. The method according to claim 20, wherein integration is by non-targeted insertion into the genome of the MAIT cell.

22. The method according to claim 20, wherein integration is by targeted insertion into a target genomic locus of the MAIT cell.

23. The method according to claim 22, wherein targeted insertion utilizes homology-directed repair.

24. The method according to claim 22 or 23, wherein the target genomic locus is an albumin gene locus, an ABO gene locus, a B2M gene locus, a CIITA gene locus, a CCR5 gene locus, a CD142 gene locus, a CLYBL gene locus, a CXCR4 gene locus, an F3 gene locus, a FUT1 gene locus, an HMGB1 gene locus, a KDM5D gene locus, an LRP1 gene locus, a MIC-A gene locus, a MIC-B gene locus, a PPP1 R12C (also known as AAVS1) gene locus, an RHD gene locus, a ROSA26 gene locus, a safe harbor gene locus, a SHS231 locus, a TAP1 gene locus, a TRAC gene locus, or a TRBC gene locus of the MAIT cell.

25. The method according to any one of the preceding claims, wherein the MAIT cell is a human cell or an animal cell.

26. The method according to any one of the preceding claims, wherein the MAIT cell is an autologous cell.

27. The method according to any one of claims 1-26, wherein the MAIT cell is an allogeneic cell.

28. The method according to any one of the preceding claims, wherein the reference cell is a comparable cell that does not comprise at least one of the one or more genetic alterations in the MAIT cell, an unmodified MAIT cell, or a wild-type MAIT cell.

29. The method according to any one of the preceding claims, wherein reduced expression comprises no cell surface expression or no detectable cell surface expression.

30. The method according to any one of the preceding claims, wherein one or more genetic alterations comprise:
(i) one or more indels,
(ii) one or more frameshift mutations, or
(iii) one or more knock-outs.

31. The method according to any one of the preceding claims, wherein one or more genetic alterations are made in:
(i) one or more coding regions, or
(ii) one or more regulatory regions.

32. The method according to any one of the preceding claims, the MAIT cell is a CAR-T cell.

33. A population of cells comprising one or more MAIT cells made according to any one of the preceding claims.

34. A cell therapy product comprising one or more MAIT cells made according to any one of claims 1-32.

35. A method comprising:
administering a cell therapy product of claim 34 to a subject.

36. A population of cells according to claim 33 or a cell therapy product according to claim 34 for use in treating a disease or condition.

37. A population of cells according to claim 33 or a cell therapy product according to claim 34 for use in the manufacture of a medicament for treating a disease or condition.

38. Use of a cell therapy product according to claim 34 for treating a disease or condition.
